(19) United States
(12) Patent Application Publication
(10) Pub. No.: US 2024/0190880 A1
(43) Pub. Date: Jun. 13, 2024

(54) SMALL MOLECULE DEGRADERS OF ESTROGEN RECEPTOR WITH CEREBLON LIGANDS

(71) Applicant: THE REGENTS OF THE UNIVERSITY OF MICHIGAN, Ann Arbor, MI (US)

(72) Inventors: Shaomeng Wang, Superior Township, MI (US); Zhixiang Chen, Ann Arbor, MI (US); Rohan Rej, Ann Arbor, MI (US); Dimin Wu, Ann Arbor, MI (US); Jianfeng Lu, Ann Arbor, MI (US); Biao Hu, Ann Arbor, MI (US); Mingliang Wang, Ann Arbor, MI (US); Ranjan Kumar Acharyya, Ann Arbor, MI (US)

(21) Appl. No.: 18/279,236

(22) PCT Filed: Mar. 4, 2022
(86) PCT No.: PCT/US22/18858

§ 371 Date: Aug. 29, 2023

Related U.S. Application Data
(60) Provisional application No. 63/156,707, filed on Mar. 4, 2021.

(51) Int. Cl. C07D 487/04; A61K 31/454; A61K 31/4545; A61K 31/4709; C07D 519/00

(52) U.S. Cl. CPC C07D 487/04; A61K 31/454; A61K 31/4545; A61K 31/4709; C07D 519/00

(57) ABSTRACT

The present disclosure relates to compounds of Formula I: and the pharmaceutically acceptable salts and solvates thereof, wherein A, X, J, Y, Z, n, and B 1 are as defined as set forth in the specification. The present disclosure also relates to uses of the compounds, e.g., in treating preventing a condition or disorder responsive to the degradation of estrogen receptor protein (e.g., cancer).

### RELATED APPLICATION

The application claims priority to, and the benefit of, U.S. Provisional Application No. 63/156,707, filed on Mar. 4, 2021, the content of which is incorporated herein by reference in its entirety.

### BACKGROUND

Breast cancer (BC) is a common malignancy in women. Based on the status of the tumor receptor, breast cancer can be further subdivided into estrogen receptor-positive (ER+), human epidermal growth factor receptor 2 (HER2)-positive (HER2+) and triple-negative subtypes. Tong et al., Front. Oncol. 2018, 8, Article 227. ER+ breast cancer occurs in approximately 80% of newly diagnosed breast cancer cases. Anderson et al., J. Natl. Cancer Inst. 2011, 103, 1397-1402. As members of the nuclear receptor family, estrogen receptors ERα and ERβ are transcription factors regulating gene expression and mediating the biological effects of the estrogens. Both ERα and ERβ are widely expressed in different tissues, and ERα is considered to be the major medium which transduces the estrogen signaling in the female reproductive tract and mammary glands. Nilsson et al., Nat. Rev. Drug Discovery 2011, 10, 778-792. ERα has thus been pursued as a therapeutic target in multiple pathological settings, particularly in cancer and osteoporosis. This is highlighted by the clinical success of tamoxifen for the treatment of ER+BC and raloxifene for the prevention and treatment of osteoporosis in postmenopausal women. Jordan, V. C., Nat. Rev. Drug Discovery 2003, 2, 205-213; Das and Crockett, Drug Des. Devel. Ther. 2013, 7, 435-448.

Although inhibition of estrogen synthesis by aromatase inhibitors and inhibition of ER pathway signaling by selective estrogen receptor modulators (SERM) have demonstrated clinical benefit in the treatment of ER+BC, the development of intrinsic and acquired resistance to these drug classes presents an impediment for patients with advanced and metastatic breast cancer. De Marchi et al., Drug Discovery Today 2016, 21, 1181-1188; AlFakeeh and Brezden-Masley, Curr. Oncol. 2018, 25, S18-S27. And while there are multiple resistance mechanisms to aromatase inhibitors and SERMs, studies have demonstrated that in most of the cases of resistance, continued dependence on ERα signaling for tumor growth and disease progression is retained and the ER protein remains a principal driver in ER+ metastatic breast cancer. Martin et al., Nat. Commun. 2017, 8, 1865; Nardone et al., Breast 2015, 24, S60-S66.

Selective estrogen receptor degraders (SERD) are small molecules that target ERα for proteasome-dependent degradation. For example, fulvestrant is a SERD that has been approved for the treatment of postmenopausal women with advanced ER+ breast cancer with standard endocrine therapies. Robertson and Harrison, Br. J. Cancer 2004, 90, S7-S10; Howell and Sapunar, Clin. Breast Cancer 2011, 11, 204-210. The proposed mechanism of action for fulvestrant (and other SERDs) is induction of misfolding of the ER protein, which ultimately leads to proteasome-dependent ERα protein degradation. Carlson, R. W., Clin. Breast Cancer 2005, 6, S5-S8. While SERDs induce degradation of ER protein in ER+ breast cancer cells, they are only able to achieve partial degradation of the ER protein. Marsaud et al., Mol. Endocrinol. 2003, 17, 2013-2027; Wittmann et al., Cancer Res. 2007, 67, 9549-9560.

Proteolysis Targeting Chimera (PROTAC) molecules are heterobifunctional conmpounds that that simultaneously bind to a target protein, e.g., ER protein, and to an E3 ligase complex, resulting in the transfer of ubiquitin and initiating a process ultimately causing the proteasomal degradation of the target protein. Benowitz et al., Expert Opinion on Therapeutic Patents 31:1-23 (2021).

There is an ongoing need for new drugs for treating and/or preventing breast cancer and other cancers and diseases responsive to the inhibition or degradation of ER proteins.

### SUMMARY

In some aspects, the present disclosure provides compounds of any one of Formulae I-VIII, below, and the pharmaceutically acceptable salts and solvates thereof.

Compounds of any one of Formula I-VIII, and the pharmaceutically acceptable salts and solvates thereof, wherein B¹ is B¹-1, B-2, B¹-3, B-4, B-5, B¹-6, or B¹-7 are collectively referred to as “Compounds of the Disclosure.” Compounds of the Disclosure are ER protein degraders.

Compounds of any one of Formula I-VIII, and the pharmaceutically acceptable salts and solvates thereof, wherein B¹ is hydrogen of hydroxy are collectively referred to as “Intermediates of the Disclosure.” Intermediates of the Disclosure are ER inhibitors and/or synthetic intermediates that can be used to prepare Compounds of the Disclosure.

In some aspects, the present disclosure provides methods of treating or preventing a condition or disease by administering a therapeutically effective amount of a Compound of the Disclosure to subject, e.g., a human, in need thereof. The disease or condition of interest is treatable or preventable by inhibition or degradation of ER proteins, for example, cancer, an inflammatory condition, or a proliferative disorder. Also provided are methods of preventing the proliferation of unwanted proliferating cells, such as in cancer, in a subject comprising administering a therapeutically effective amount of a Compound of the Disclosure to a subject at risk of developing a condition characterized by unwanted proliferating cells. In some embodiments, the Compounds of the Disclosure reduce the proliferation of unwanted cells by inducing apoptosis in those cells.

In some aspects, the present disclosure provides a method of reducing one or more ER proteins with a cell of a subject in need thereof, the method comprising administering to the subject a therapeutically effective amount of at least one Compound of the Disclosure.

In some aspects, the present disclosure provides a pharmaceutical composition comprising a Compound of the Disclosure and an excipient and/or pharmaceutically acceptable carrier.

In some aspects, the present disclosure provides a composition comprising a Compound of the Disclosure and an excipient and/or pharmaceutically acceptable carrier for use treating or preventing diseases or conditions wherein the degradation of ER proteins provides a benefit, e.g., cancer.

In some aspects, the present disclosure provides a composition comprising: (a) a Compound of the Disclosure; (b) a second therapeutically active agent; and (c) optionally an excipient and/or pharmaceutically acceptable carrier.

In some aspects, the present disclosure provides a Compound of the Disclosure for use in treatment or prevention of a disease or condition of interest, e.g., cancer.

In some aspects, the present disclosure provides a use of a Compound of the Disclosure for the manufacture of a medicament for treating a disease or condition of interest, e.g., cancer.

In some aspects, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In some aspects, the present disclosure provides methods of preparing Compounds of the Disclosure and Intermediates of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

It is to be understood that both the foregoing summary and the following detailed description are exemplary and explanatory only, and are not restrictive of the invention as claimed.

### DETAILED DESCRIPTION

The present disclosure relates to compounds which may function as estrogen receptor (ER) protein degraders. The present disclosure also relates to uses of the compounds in treating or preventing conditions and diseases, e.g., wherein the degradation of ER proteins provides a benefit.

Compounds and Intermediates of the Disclosure

In some aspects, the present disclosure provides compounds of Formula I:

A-X-J-Y—Z—(CH₂)ₙ—B¹ I,

or a pharmaceutically acceptable salt or solvate thereof, wherein:
A is selected from:

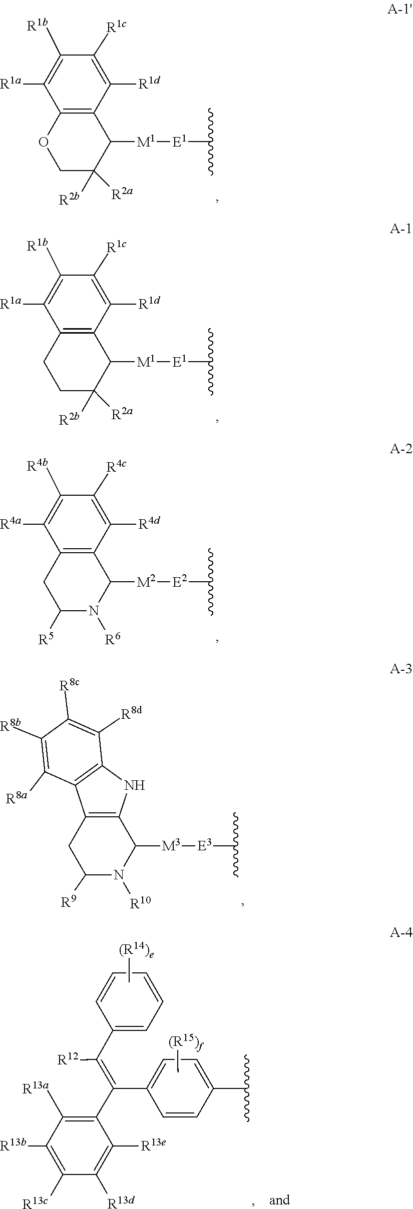

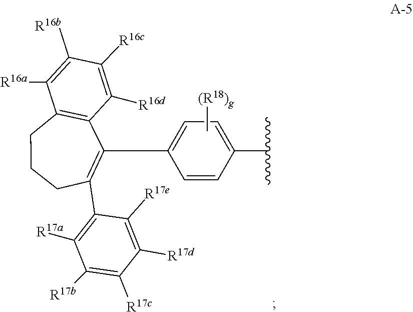

M¹ is selected from 4- to 8-membered heterocyclenyl, phenylenyl, 5-membered heteroarylenyl, and 6-membered heteroarylenyl, wherein the 4- to 8-membered heterocyclenyl, phenylenyl, 5-membered heteroarylenyl, or 6-membered heteroarylenyl is optionally substituted with one or more halo or C₁-C₃ alkoxy;
R^{1a}, R^{1b}, R^{1c}, and R^{1d} are independently selected from hydrogen, halo, hydroxy, 5-membered heteroayl, and —B(OH)₂, wherein the 5-membered heteroayl is optionally substituted with one or more C₁-C₄ alkyl; or
R^{1a} and R^{1b} taken together with the carbon atom to which they are attached form an optionally substituted 5- or 6-membered heteroaryl; and R^{1c} and R^{1d} are hydrogen;
R^{2a} is selected from optionally substituted phenyl and optionally substituted C₃-C₈ cycloalkyl; and R^{2b} is hydrogen; or
R^{2a} and R^{2b} taken together with the carbon atom to which they are attached form a C₃-C₈ cycloalkyl;
E¹ is selected from —C(═O)—, —C≡C—, —O—, —O—(CH₂)_{b}—, N(R^{3e})—, and —(CH₂)_{b}—;
R^{3e} is selected from hydrogen and C₁-C₄ alkyl;
b is 0, 1, 2, 3, 4, or 5;
M² is selected from 4- to 8-membered heterocyclenyl, phenylenyl, 5-membered heteroarylenyl, and 6-membered heteroarylenyl, wherein the 4- to 8-membered heterocyclenyl, phenylenyl, 5-membered heteroarylenyl, or 6-membered heteroarylenyl is optionally substituted with one or more halo or C₁-C₃ alkoxy;
R^{4a}, R^{4b}, R^{4c}, and R^{4d} are independently selected from hydrogen, halo, hydroxy, and —B(OH)₂; or
R^{4a} and R^{4b} taken together with the carbon atoms to which they are attached form an optionally substituted 5- or 6-membered heteroaryl; and R^{4c} and R^{4d} are hydrogen;
R⁵ is C₁-C₃ alkyl;
R⁶ is C₁-C₄ haloalkyl;
E² is selected from —C(═O)—, —C≡C—, —O—, —O—(CH₂)_{c}—, —N(R^{7e})—, and —(CH₂)_{c}—;
R^{7e} is selected from hydrogen and C₁-C₄ alkyl;
c is 0, 1, 2, 3, 4, or 5;
M³ is selected from 4- to 8-membered heterocyclenyl, phenylenyl, 5-membered heteroarylenyl, and 6-membered heteroarylenyl, wherein the 4- to 8-membered heterocyclenyl, phenylenyl, 5-membered heteroarylenyl, or 6-membered heteroarylenyl is optionally substituted with one or more halo or C₁-C₃ alkoxy;
R^{8a}, R^{8b}, R^{8c}, and R^{8d} are independently selected from hydrogen, halo, hydroxy, and —B(OH)₂;
R⁹ is C₁-C₃ alkyl;
R¹⁰ is C₁-C₄ haloalkyl optionally substituted with one or more hydroxy, or —(C₁-C₄ alkyl)-(C₃-C₈ cycloalkyl) optionally substituted with one or more halo;
E³ is selected from —C(═O)—, —C≡C—, —O—, —O—(CH₂)_{d}—, N(R^{11e})—, and —(CH₂)_{d}—;
R^{11e} is selected from hydrogen and C₁-C₄ alkyl;
d is 0, 1, 2, 3, 4, or 5;
R¹² is selected from C₁-C₄ alkyl and C₁-C₄ haloalkyl;
R^{13a}, R^{13b}, R^{13c}, and R^{13d} are independently selected from hydrogen, halo, hydroxy, and —B(OH)₂;
each R¹⁴ is independently selected from hydrogen, halo, and hydroxy;
e is 0, 1, 2, or 3;
each R¹⁵ is independently selected from hydrogen, halo, and hydroxy;
f is 0, 1, 2, or 3;
R^{16a}, R^{16b}, R^{16c}, and R^{16d} are independently selected from hydrogen, halo, hydroxy, and —B(OH)₂;
R^{17a}, R^{17b}, R^{17c}, R^{17d}, and R^{17e} are independently selected from hydrogen, halo, and hydroxy;
each R¹⁸ is independently selected from hydrogen, halo, and hydroxy;
g is 0, 1, 2, or 3;
X is selected from cycloalkylenyl, heterocyclenyl, phenylenyl, and heteroarylenyl, wherein the cycloalkylenyl, heterocyclenyl, phenylenyl, or heteroarylenyl is optionally substituted with one or more C₁-C₄ alkyl;
J is selected from —C(═O)—, —(CH₂)ₘ—, —(CH₂)_{z1}N(R¹⁹)—, and —(CH₂)_{z2}O—,
   m is 0, 1, 2, or 3;
   z1 is 0, 1, or 2;
   z2 is 0, 1, or 2;
   R¹⁹ is selected from hydrogen and C₁-C₄ alkyl;
Y is selected from cycloalkylenyl, heterocyclenyl, heteroarylenyl, —C(═O)— and —(CR^{20a}R^{20b})ᵣ—;
Z is selected from cycloalkylenyl, heterocyclenyl, heteroarylenyl, —C(═O)— and —(CR^{20c}R^{20d})ₛ—;
   each R^{20a}, R^{20b}, R^{20c}, and R^{20d} is independently selected from hydrogen and C₁-C₃ alkyl;
   r is 0, 1, 2, 3, 4, or 5;
   s is 0, 1, 2, 3, 4, or 5;
with the provisos:
   (i) Z is cycloalkylenyl, heterocyclenyl, heteroarylenyl, or —(CR^{20c}R^{20d})ₛ— when Y is —C(═O)—; or
   (ii) Y is cycloalkylenyl, heterocyclenyl, heteroarylenyl, or —(CR^{20a}R^{20b})ᵣ— when Z is —C(═O)—;
n is 0, 1, 2, or 3;
B¹ is selected from hydrogen, hydroxy,

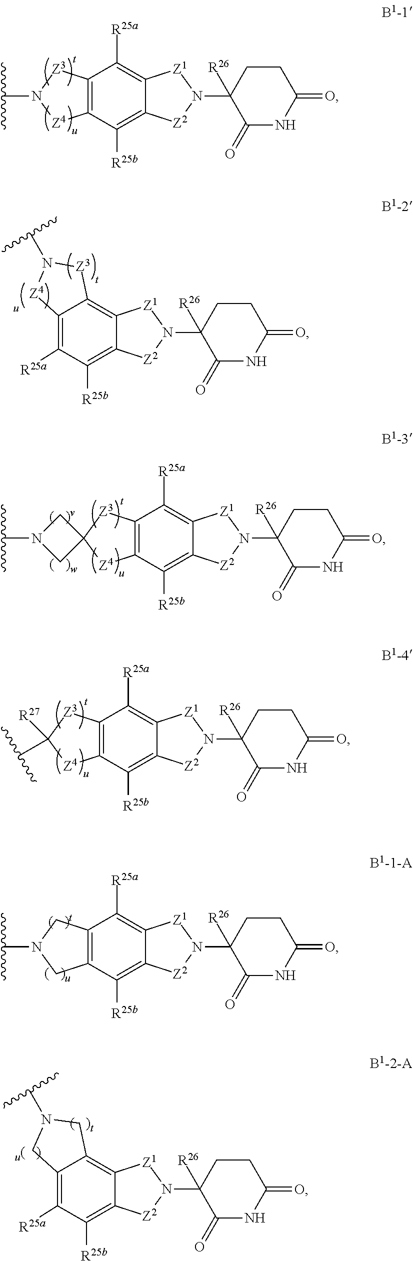

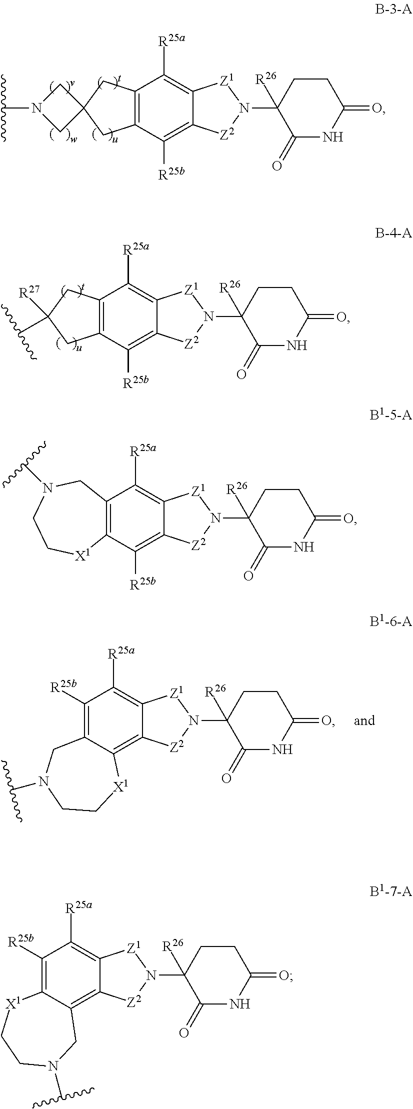

R^{25a} and R^{25b} are independently selected from hydrogen, amino, halo, C₁-C₃ alkyl, and C₁-C₃ alkoxy;
R²⁶ is selected from hydrogen, deuterium, fluoro, and C₁-C₃ alkyl;
R²⁷ is selected from hydrogen and C₁-C₃ alkyl;
Z¹ and Z² are independently selected from —C(═O)— and —CR^{28a}R^{28b}—; with the provisos:
   (iv) one of Z¹ or Z² is —C(═O)—; or
   (v) both of Z¹ and Z² are —C(═O)—;
R^{28a} and R^{28b} are independently selected from hydrogen and C₁-C₃ alkyl; or
R^{28a} and R^{28b} taken together with the carbon atom to which they are attached form a C₃-C₆ cycloalkyl;
Z³ and Z⁴ are independently selected from —C(═O)— and —CR^{28c}R^{28d}—; with the provisos:
   (iv) one of Z³ or Z⁴ is —C(═O)—; or
   (v) both of Z³ and Z⁴ are —C(═O)—;
R^{28c} and R^{28d} are independently selected from hydrogen and C₁-C₃ alkyl; or
R^{28c} and R^{28d} taken together with the carbon atom to which they are attached form a C₃-C₆ cycloalkyl;
X¹ is selected from —O—, —S—, and —N(R²⁹)—;
R²⁹ is selected from hydrogen and C₁-C₄ alkyl;
t is 1, 2, or 3;
u is 1, 2, or 3;
v is 1, 2, or 3; and
w is 1, 2, or 3.

In some aspects, the disclosure provides compounds of Formula I:

A-X-J-Y—Z—(CH₂)ₙ—B¹ I,

or a pharmaceutically acceptable salt or solvate thereof, wherein:
A is selected from:

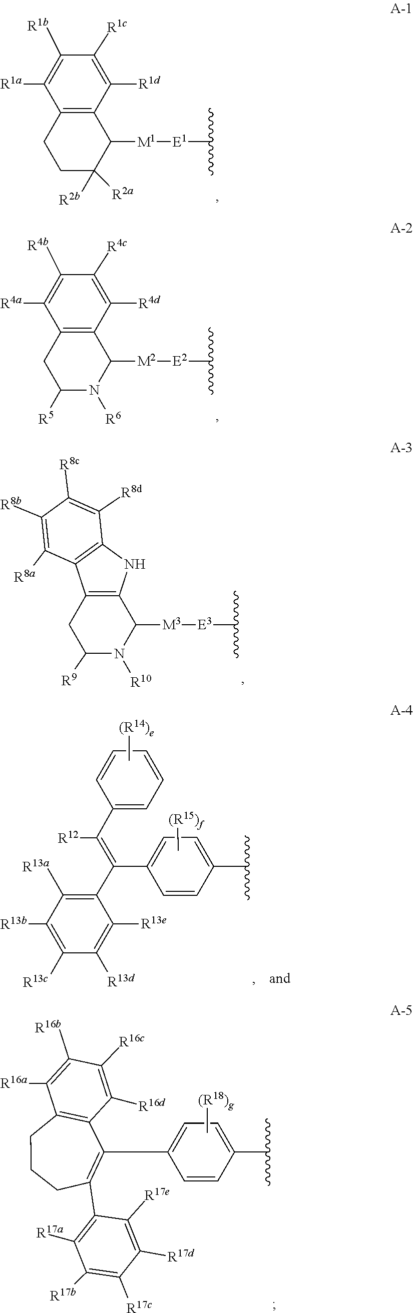

M¹ is selected from 4- to 8-membered heterocyclenyl, phenylenyl, 5-membered heteroarylenyl, and 6-membered heteroarylenyl;
R^{1a}, R^{1b}, R^{1c}, and R^{1d} are independently selected from hydrogen, halo, hydroxy, and —B(OH)₂;
R^{2a} is selected from optionally substituted phenyl and optionally substituted C₃-C₈ cycloalkyl; and R^{2b} is hydrogen; or
R^{2a} and R^{2b} taken together with the carbon atom to which they are attached form a C₃-C₈ cycloalkyl;
E¹ is selected from —C≡C—, —O—, N(R^{3e})—, and —(CH₂)_{b}—;
R^{3e} is selected from hydrogen and C₁-C₄ alkyl;
b is 0, 1, 2, 3, 4, or 5;
M² is selected from 4- to 8-membered heterocyclenyl, phenylenyl, 5-membered heteroarylenyl, and 6-membered heteroarylenyl;
R^{4a}, R^{4b}, R^{4c}, and R^{4d} are independently selected from hydrogen, halo, hydroxy, and —B(OH)₂; or
R^{4a} and R^{4b} taken together with the carbon atoms to which they are attached form an optionally substituted 5- or 6-membered heteroaryl; and R^{4c} and R^{4d} are hydrogen;
R⁵ is C₁-C₃ alkyl;
R⁶ is C₁-C₄ haloalkyl;
E² is selected from —C≡C—, —O—, —N(R^{7e})—, and —(CH₂)_{c}—;
R^{7e} is selected from hydrogen and C₁-C₄ alkyl;
c is 0, 1, 2, 3, 4, or 5;
M³ is selected from 4- to 8-membered heterocyclenyl, phenylenyl, 5-membered heteroarylenyl, and 6-membered heteroarylenyl;
R^{8a}, R^{8b}, R^{8c}, and R^{8d} are independently selected from hydrogen, halo, hydroxy, and —B(OH)₂;
R⁹ is C₁-C₃ alkyl;
R¹⁰ is C₁-C₄ haloalkyl;
E³ is selected from —C≡C—, —O—, N(R^{11e})—, and —(CH₂)_{d}—;
R^{11e} is selected from hydrogen and C₁-C₄ alkyl;
d is 0, 1, 2, 3, 4, or 5;
R¹² is selected from C₁-C₄ alkyl and C₁-C₄ haloalkyl;
R^{13a}, R^{13b}, R^{13c}, and R^{13d} are independently selected from hydrogen, halo, hydroxy, and —B(OH)₂;
each R¹⁴ is independently selected from hydrogen, halo, and hydroxy;
e is 0, 1, 2, or 3;
each R¹⁵ is independently selected from hydrogen, halo, and hydroxy;
f is 0, 1, 2, or 3;
R^{16a}, R^{16b}, R^{16c}, and R^{16d} are independently selected from hydrogen, halo, hydroxy, and —B(OH)₂;
R^{17a}, R^{17b}, R^{17c}, R^{17d}, and R^{17e} are independently selected from hydrogen, halo, and hydroxy;
each R¹⁹ is independently selected from hydrogen, halo, and hydroxy;
g is 0, 1, 2, or 3;
X is selected from cycloalkylenyl, heterocyclenyl, phenylenyl, and heteroarylenyl;
J is selected from —(CH₂)ₘ—, —(CH₂)_{z1}N(R¹⁹)—, and —(CH₂)_{z2}O—,
   m is 0, 1, 2, or 3;
   z1 is 0, 1, or 2;
   z2 is 0, 1, or 2;
   R¹⁹ is selected from hydrogen and C₁-C₄ alkyl;
Y is selected from cycloalkylenyl, heterocyclenyl, heteroarylenyl, —C(═O)— and —(CR^{20a}R^{20b})ᵣ—;
Z is selected from cycloalkylenyl, heterocyclenyl, heteroarylenyl, —C(═O)— and —(CR^{20c}R^{20d})ₛ—;
   each R^{20a}, R^{20b}, R^{20c}, and R^{20d} is independently selected from hydrogen and C₁-C₃ alkyl;
   r is 0, 1, 2, 3, 4, or 5;
   s is 0, 1, 2, 3, 4, or 5;
with the provisos:
   (i) Z is —(CR^{20c}R^{20d})ₛ— when Y is —C(═O)—; or
   (ii) Y is —(CR^{20a}R^{20b})ᵣ— when Z is —C(═O)—;
n is 0, 1, 2, or 3;
B¹ is selected from hydrogen, hydroxy,

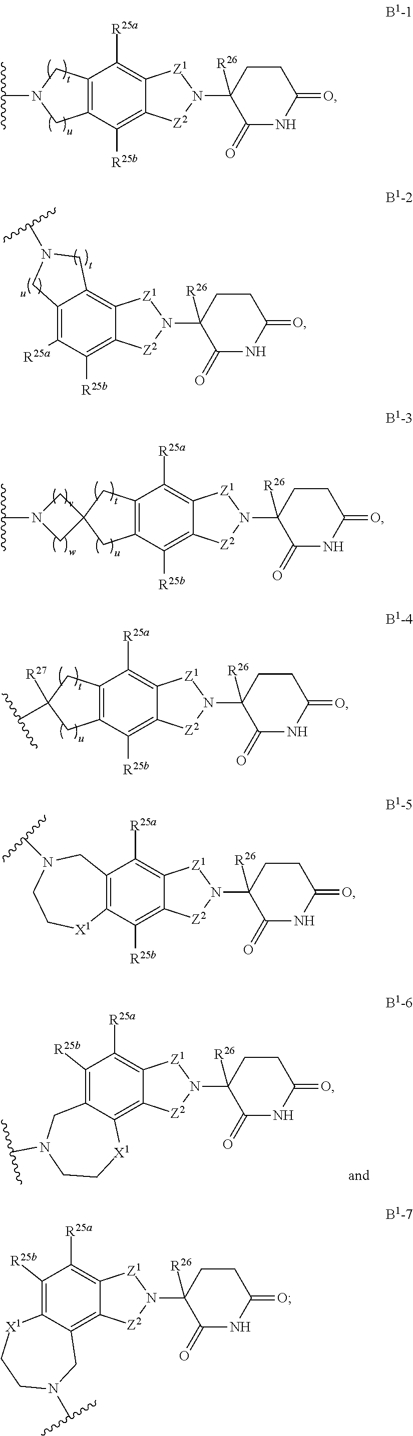

R^{25a} and R^{25b} are independently selected from hydrogen, amino, halo, C₁-C₃ alkyl, and C₁-C₃ alkoxy;
R²⁶ is selected from hydrogen, deuterium, fluoro, and C₁-C₃ alkyl;
R²⁷ is selected from hydrogen and C₁-C₃ alkyl;
Z¹ and Z² are independently selected from —C(═O)— and —CR^{28a}R^{28b}—; with the provisos:
   (iv) one of Z¹ or Z² is —C(═O)—; or
   (v) both of Z¹ and Z² are —C(═O)—;
R^{28a} and R^{28b} are independently selected from hydrogen and C₁-C₃ alkyl; or
R^{28a} and R^{28b} taken together with the carbon atom to which they are attached form a C₃-C₆ cycloalkyl;
X¹ is selected from —O—, —S—, and —N(R²⁹)—;
R²⁹ is selected from hydrogen and C₁-C₄ alkyl;
t is 1, 2, or 3;
u is 1, 2, or 3;
v is 1, 2, or 3; and
w is 1, 2, or 3.

In some embodiments, A is

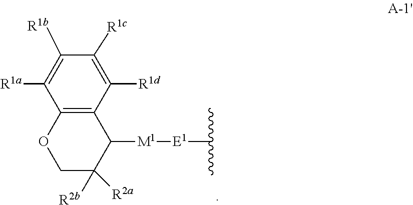

In some embodiments, A is

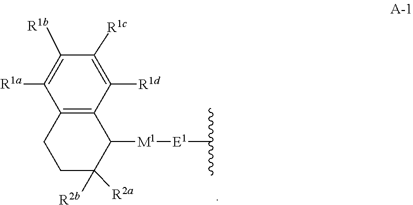

In some embodiments, A is

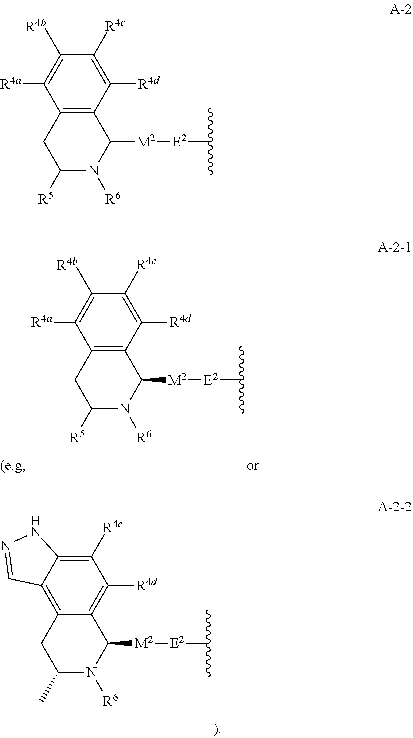

In some embodiments, A is

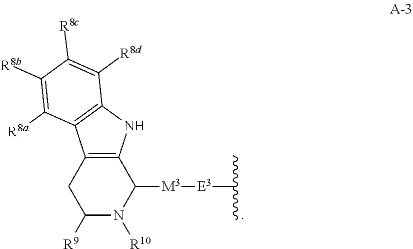

In some embodiments, A is

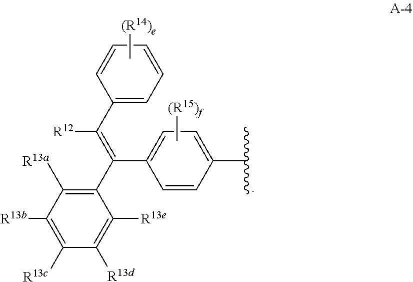

In some embodiments, A is

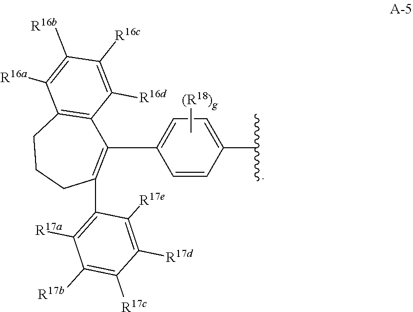

In some embodiments, M¹ is 4- to 8-membered heterocyclenyl optionally substituted with one or more halo or C₁-C₃ alkoxy.

In some embodiments, M¹ is phenylenyl optionally substituted with one or more halo or C₁-C₃ alkoxy.

In some embodiments, M¹ is 5-membered heteroarylenyl optionally substituted with one or more halo or C₁-C₃ alkoxy.

In some embodiments, M¹ is 6-membered heteroarylenyl optionally substituted with one or more halo or C₁-C₃ alkoxy.

In some embodiments, R^{1a} is hydrogen.

In some embodiments, R^{1a} is halo.

In some embodiments, R^{1a} is hydroxy.

In some embodiments, R^{1a} is —B(OH)₂.

In some embodiments, R^{1a} is 5-membered heteroayl optionally substituted with one or more C₁-C₄ alkyl.

In some embodiments, R^{1b} is hydrogen.

In some embodiments, R^{1b} is halo.

In some embodiments, R^{1b} is hydroxy.

In some embodiments, R^{1b} is —B(OH)₂.

In some embodiments, R^{1b} is 5-membered heteroayl optionally substituted with one or more C₁-C₄ alkyl.

In some embodiments, R^{1c} is hydrogen.

In some embodiments, R^{1c} is halo.

In some embodiments, R^{1c} is hydroxy.

In some embodiments, R^{1c} is —B(OH)₂.

In some embodiments, R^{1c} is 5-membered heteroayl optionally substituted with one or more C₁-C₄ alkyl.

In some embodiments, R^{1d} is hydrogen.

In some embodiments, R^{1d} is halo.

In some embodiments, R^{1d} is hydroxy.

In some embodiments, R^{1d} is —B(OH)₂.

In some embodiments, R^{1d} is 5-membered heteroayl optionally substituted with one or more C₁-C₄ alkyl.

In some embodiments, R^{1a} and R^{1b} taken together with the carbon atom to which they are attached form an optionally substituted 5- or 6-membered heteroaryl; and R^{1c} and R^{1d} are hydrogen.

In some embodiments, R^{2a} is optionally substituted phenyl, and R^{2b} is hydrogen.

In some embodiments, R^{2a} is optionally substituted C₃-C₈ cycloalkyl, and R^{2b} is hydrogen.

In some embodiments, R^{2a} and R^{2b} taken together with the carbon atom to which they are attached form a C₃-C₈ cycloalkyl.

In some embodiments, E¹ is —C(═O)—.

In some embodiments, E¹ is —C≡C—.

In some embodiments, E¹ is —O—.

In some embodiments, E¹ is —O—(CH₂)_{b}—.

In some embodiments, E¹ is —N(R^{3e})—.

In some embodiments, E¹ is —(CH₂)_{b}—.

In some embodiments, R^{3e} is hydrogen.

In some embodiments, R^{3e} is C₁-C₄ alkyl.

In some embodiments, b is 0.

In some embodiments, b is 1.

In some embodiments, b is 2.

In some embodiments, b is 3.

In some embodiments, b is 4.

In some embodiments, b is 5.

In some embodiments, M² is 4- to 8-membered heterocyclenyl optionally substituted with one or more halo or C₁-C₃ alkoxy.

In some embodiments, M² is phenylenyl optionally substituted with one or more halo or C₁-C₃ alkoxy.

In some embodiments, M² is 5-membered heteroarylenyl optionally substituted with one or more halo or C₁-C₃ alkoxy.

In some embodiments, M² is 6-membered heteroarylenyl optionally substituted with one or more halo or C₁-C₃ alkoxy.

In some embodiments, R^{4a} is hydrogen.

In some embodiments, R^{4a} is halo.

In some embodiments, R^{4a} is hydroxy.

In some embodiments, R^{4a} is —B(OH)₂.

In some embodiments, R^{4b} is hydrogen.

In some embodiments, R^{4b} is halo.

In some embodiments, R^{4b} is hydroxy.

In some embodiments, R^{4b} is —B(OH)₂.

In some embodiments, R^{4c} is hydrogen.

In some embodiments, R^{4c} is halo.

In some embodiments, R^{4c} is hydroxy.

In some embodiments, R^{4c} is —B(OH)₂.

In some embodiments, R^{4d} is hydrogen.

In some embodiments, R^{4d} is halo.

In some embodiments, R^{4d} is hydroxy.

In some embodiments, R^{4d} is —B(OH)₂.

In some embodiments, R^{4a} and R^{4b} taken together with the carbon atoms to which they are attached form an optionally substituted 5- or 6-membered heteroaryl; and R^{4c} and R^{4d} are hydrogen.

In some embodiments, R⁵ is C₁-C₃ alkyl.

In some embodiments, R⁶ is C₁-C₄ haloalkyl.

In some embodiments, E² is —C(═O)—.

In some embodiments, E² is —C≡C—.

In some embodiments, E² is —O—.

In some embodiments, E² is —O—(CH₂)_{c}—.

In some embodiments, E² is —N(R^{7e})—.

In some embodiments, E² is —(CH₂)_{c}—.

In some embodiments, R^{7e} is hydrogen.

In some embodiments, R^{7e} is C₁-C₄ alkyl.

In some embodiments, c is 0.

In some embodiments, c is 1.

In some embodiments, c is 2.

In some embodiments, c is 3.

In some embodiments, c is 4.

In some embodiments, c is 5.

In some embodiments, M³ is 4- to 8-membered heterocyclenyl optionally substituted with one or more halo or C₁-C₃ alkoxy.

In some embodiments, M³ is phenylenyl optionally substituted with one or more halo or C₁-C₃ alkoxy.

In some embodiments, M³ is 5-membered heteroarylenyl optionally substituted with one or more halo or C₁-C₃ alkoxy.

In some embodiments, M³ is 6-membered heteroarylenyl optionally substituted with one or more halo or C₁-C₃ alkoxy.

In some embodiments, R^{8a} is hydrogen.

In some embodiments, R^{8a} is halo.

In some embodiments, R^{8a} is hydroxy.

In some embodiments, R^{8a} is —B(OH)₂.

In some embodiments, R^{8b} is hydrogen.

In some embodiments, R^{8b} is halo.

In some embodiments, R^{8b} is hydroxy.

In some embodiments, R^{8b} is —B(OH)₂.

In some embodiments, R^{8c} is hydrogen.

In some embodiments, R^{8c} is halo.

In some embodiments, R^{8c} is hydroxy.

In some embodiments, R^{8c} is —B(OH)₂.

In some embodiments, R^{8d} is hydrogen.

In some embodiments, R^{8d} is halo.

In some embodiments, R^{8d} is hydroxy.

In some embodiments, R^{8d} is —B(OH)₂.

In some embodiments, R⁹ is C₁-C₃ alkyl;

In some embodiments, R¹⁰ is C₁-C₄ haloalkyl optionally substituted with one or more hydroxy.

In some embodiments, R¹⁰ is —(C₁-C₄ alkyl)-(C₃-C₅ cycloalkyl) optionally substituted with one or more halo.

In some embodiments, E³ is —C(═O)—.

In some embodiments, E³ is —C≡C—.

In some embodiments, E³ is —O—.

In some embodiments, E³ is —O—(CH₂)_{d}—.

In some embodiments, E³ is —N(R^{11e})—.

In some embodiments, E³ is —(CH₂)_{d}—.

In some embodiments, Rile is hydrogen.

In some embodiments, Rile is C₁-C₄ alkyl.

In some embodiments, d is 0.

In some embodiments, d is 1.

In some embodiments, d is 2.

In some embodiments, d is 3.

In some embodiments, d is 4.

In some embodiments, d is 5.

In some embodiments, R¹² is C₁-C₄ alkyl.

In some embodiments, R¹² is C₁-C₄ haloalkyl.

In some embodiments, R^{13a} is hydrogen.

In some embodiments, R^{13a} is halo.

In some embodiments, R^{13a} is hydroxy.

In some embodiments, R^{13a} is —B(OH)₂.

In some embodiments, R^{13b} is hydrogen.

In some embodiments, R^{13b} is halo.

In some embodiments, R^{13b} is hydroxy.

In some embodiments, R^{13b} is —B(OH)₂.

In some embodiments, R^{13c} is hydrogen.

In some embodiments, R^{13c} is halo.

In some embodiments, R^{13c} is hydroxy.

In some embodiments, R^{13c} is —B(OH)₂.

In some embodiments, R^{13d} is hydrogen.

In some embodiments, R^{13d} is halo.

In some embodiments, R^{13d} is hydroxy.

In some embodiments, R^{13d} is —B(OH)₂.

In some embodiments, at least one R¹⁴ is hydrogen.

In some embodiments, at least one R¹⁴ is halo.

In some embodiments, at least one R¹⁴ is hydroxy.

In some embodiments, e is 0.

In some embodiments, e is 1.

In some embodiments, e is 2.

In some embodiments, e is 3.

In some embodiments, at least one R¹⁵ is hydrogen.

In some embodiments, at least one R¹⁵ is halo.

In some embodiments, at least one R¹⁵ is hydroxy.

In some embodiments, f is 0.

In some embodiments, f is 1.

In some embodiments, f is 2.

In some embodiments, f is 3.

In some embodiments, R^{16a} is hydrogen.

In some embodiments, R^{16a} is halo.

In some embodiments, R^{16a} is hydroxy.

In some embodiments, R^{16a} is —B(OH)₂.

In some embodiments, R^{16b} is hydrogen.

In some embodiments, R^{16b} is halo.

In some embodiments, R^{16b} is hydroxy.

In some embodiments, R^{16b} is —B(OH)₂.

In some embodiments, R^{16c} is hydrogen.

In some embodiments, R^{16c} is halo.

In some embodiments, R^{16c} is hydroxy.

In some embodiments, R¹⁶, is —B(OH)₂.

In some embodiments, R^{16d} is hydrogen.

In some embodiments, R^{16d} is halo.

In some embodiments, R^{16d} is hydroxy.

In some embodiments, R^{16d} is —B(OH)₂.

In some embodiments, R^{17a} is hydrogen.

In some embodiments, R^{17a} is halo.

In some embodiments, R^{17a} is hydroxy.

In some embodiments, R^{17b} is hydrogen.

In some embodiments, R^{17b} is halo.

In some embodiments, R^{17b} is hydroxy.

In some embodiments, R^{17c} is hydrogen.

In some embodiments, R^{17c} is halo.

In some embodiments, R^{17c} is hydroxy.

In some embodiments, R^{17d} is hydrogen.

In some embodiments, R^{17d} is halo.

In some embodiments, R^{17d} is hydroxy.

In some embodiments, R^{17e} is hydrogen.

In some embodiments, R^{17e} is halo.

In some embodiments, R^{17e} is hydroxy.

In some embodiments, at least one R¹⁸ is hydrogen.

In some embodiments, at least one R¹⁸ is halo.

In some embodiments, at least one R¹⁸ is hydroxy.

In some embodiments, g is 0.

In some embodiments, g is 1.

In some embodiments, g is 2.

In some embodiments, g is 3.

In some embodiments, X is cycloalkylenyl optionally substituted with one or more C₁-C₄ alkyl.

In some embodiments, X is heterocyclenyl optionally substituted with one or more C₁-C₄ alkyl.

In some embodiments, X is phenylenyl optionally substituted with one or more C₁-C₄ alkyl.

In some embodiments, X is heteroarylenyl optionally substituted with one or more C₁-C₄ alkyl.

In some embodiments, J is —C(═O)—.

In some embodiments, J is —(CH₂)ₘ—.

In some embodiments, J is —(CH₂)_{z1}N(R¹⁹)—.

In some embodiments, J is —(CH₂)_{z2}O—.

In some embodiments, m is 0.

In some embodiments, m is 1.

In some embodiments, m is 2.

In some embodiments, m is 3.

In some embodiments, z1 is 0.

In some embodiments, z1 is 1.

In some embodiments, z1 is 2.

In some embodiments, z2 is 0.

In some embodiments, z2 is 1.

In some embodiments, z2 is 2.

In some embodiments, R¹⁹ is hydrogen.

In some embodiments, R¹⁹ is C₁-C₄ alkyl.

In some embodiments, Y is cycloalkylenyl.

In some embodiments, Y is heterocyclenyl.

In some embodiments, Y is heteroarylenyl.

In some embodiments, Y is —C(═O)—.

In some embodiments, Y is —(CR^{20a}R^{20b})ᵣ—.

In some embodiments, Z is cycloalkylenyl.

In some embodiments, Z is heterocyclenyl.

In some embodiments, Z is heteroarylenyl.

In some embodiments, Z is —C(═O)—.

In some embodiments, Z is —(CR^{20c}R^{20d})ₛ—.

In some embodiments, R^{20a} is hydrogen.

In some embodiments, R^{20a} is C₁-C₃ alkyl.

In some embodiments, R^{20b} is hydrogen.

In some embodiments, R^{20b} is C₁-C₃ alkyl.

In some embodiments, R^{20c} is hydrogen.

In some embodiments, R^{20c} is C₁-C₃ alkyl.

In some embodiments, R^{20d} is hydrogen.

In some embodiments, R^{20d} is C₁-C₃ alkyl.

In some embodiments, r is 0.

In some embodiments, r is 1.

In some embodiments, r is 2.

In some embodiments, r is 3.

In some embodiments, r is 4.

In some embodiments, r is 5.

In some embodiments, s is 0.

In some embodiments, s is 1.

In some embodiments, s is 2.

In some embodiments, s is 3.

In some embodiments, s is 4.

In some embodiments, s is 5.

In some embodiments, (i) Z is cycloalkylenyl, heterocyclenyl, heteroarylenyl, or —(CR^{20c}R^{20d})ₛ— when Y is —C(═O)—; and (ii) Y is cycloalkylenyl, heterocyclenyl, heteroarylenyl, or —(CR^{20a}R^{20b})ᵣ— when Z is —C(═O)—.

In some embodiments, Z is cycloalkylenyl, heterocyclenyl, heteroarylenyl, or —(CR^{20c}R^{20d})ₛ— and Y is —C(═O)—.

In some embodiments, Y is cycloalkylenyl, heterocyclenyl, heteroarylenyl, or —(CR^{20a}R^{20b})ᵣ— and Z is —C(═O)—.

In some embodiments, (i) Z is —(CR^{20c}R^{20d})ₛ— when Y is —C(═O)—; and (ii) Y is —(CR^{20a}R^{20b})ᵣ— when Z is —C(═O)—.

In some embodiments, Z is —(CR^{20c}R^{20d})ₛ— and Y is —C(═O)—.

In some embodiments, Y is —(CR^{20a}R^{20b})ᵣ— and Z is —C(═O)—.

In some embodiments, n is 0.

In some embodiments, n is 1.

In some embodiments, n is 2.

In some embodiments, n is 3.

In some embodiments, B¹ is hydrogen.

In some embodiments, B¹ is hydroxy.

In some embodiments, B¹ is

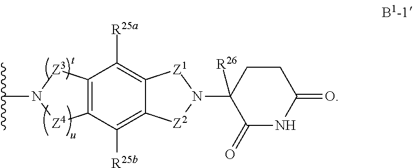

In some embodiments, B¹ is

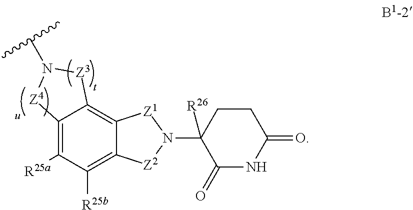

In some embodiments, B¹ is

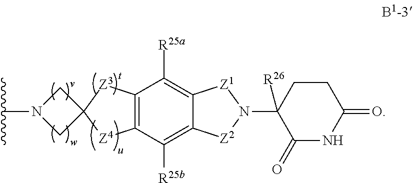

In some embodiments, B¹ is

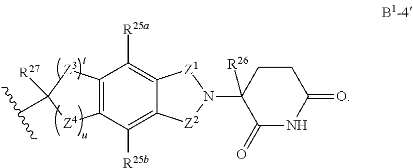

In some embodiments, B¹ is

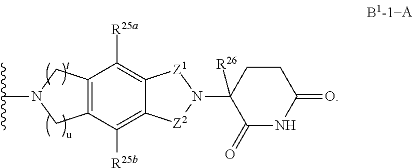

In some embodiments, B¹ is

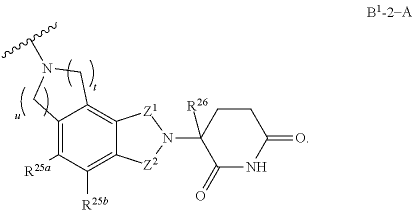

In some embodiments, B¹ is

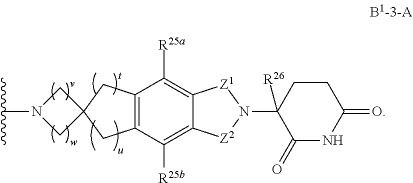

In some embodiments, B¹ is

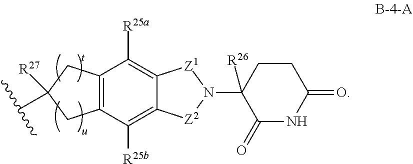

In some embodiments, B¹ is

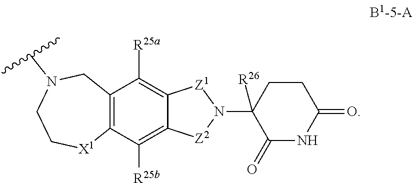

In some embodiments, B¹ is

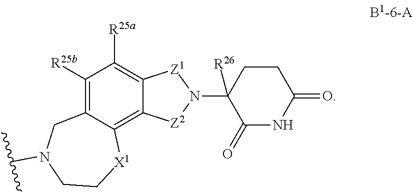

In some embodiments, B¹ is

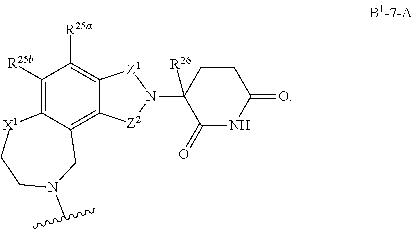

In some embodiments, R^{25a} is hydrogen.

In some embodiments, R^{25a} is amino.

In some embodiments, R^{25a} is halo.

In some embodiments, R^{25a} is C₁-C₃ alkyl.

In some embodiments, R^{25a} is C₁-C₃ alkoxy.

In some embodiments, R^{25b} is hydrogen.

In some embodiments, R^{25b} is amino.

In some embodiments, R^{25b} is halo.

In some embodiments, R^{25b} is C₁-C₃ alkyl.

In some embodiments, R^{25b} is C₁-C₃ alkoxy.

In some embodiments, R²⁶ is hydrogen.

In some embodiments, R²⁶ is deuterium.

In some embodiments, R²⁶ is fluoro.

In some embodiments, R²⁶ is C₁-C₃ alkyl.

In some embodiments, R²⁷ is hydrogen.

In some embodiments, R²⁷ is C₁-C₃ alkyl.

In some embodiments, Z¹ is —C(═O)—.

In some embodiments, Z¹ is —CR^{28a}R^{28b}—.

In some embodiments, Z² is —C(═O)—.

In some embodiments, Z² is —CR^{28a}R^{28b}—.

In some embodiments, one of Z¹ or Z² is —C(═O)—.

In some embodiments, both of Z¹ and Z² are —C(═O)—.

In some embodiments, R^{28a} is hydrogen.

In some embodiments, R^{28a} is C₁-C₃ alkyl.

In some embodiments, R^{28b} is hydrogen.

In some embodiments, R^{28b} is C₁-C₃ alkyl.

In some embodiments, R^{28a} and R^{28b} taken together with the carbon atom to which they are attached form a C₃-C₆ cycloalkyl.

In some embodiments, Z³ is —C(═O)—.

In some embodiments, Z³ is —CR^{28c}R^{28d}—.

In some embodiments, Z⁴ is —C(═O)—.

In some embodiments, Z⁴ is —CR^{28c}R^{28d}—.

In some embodiments, one of Z³ or Z⁴ is —C(═O)—.

In some embodiments, both of Z³ and Z⁴ are —C(═O)—.

In some embodiments, R^{28c} is hydrogen.

In some embodiments, R^{28c} is C₁-C₃ alkyl.

In some embodiments, R^{28d} is hydrogen.

In some embodiments, R^{28d} is C₁-C₃ alkyl.

In some embodiments, R^{28c} and R^{28d} taken together with the carbon atom to which they are attached form a C₃-C₆ cycloalkyl.

In some embodiments, X¹ is —O—.

In some embodiments, X¹ is —S—.

In some embodiments, X¹ is —N(R²⁹)—.

In some embodiments, R²⁹ is hydrogen.

In some embodiments, R²⁹ is C₁-C₄ alkyl.

In some embodiments, t is 1.

In some embodiments, t is 2.

In some embodiments, t is 3.

In some embodiments, u is 1.

In some embodiments, u is 2.

In some embodiments, u is 3.

In some embodiments, v is 1.

In some embodiments, v is 2.

In some embodiments, v is 3.

In some embodiments, w is 1.

In some embodiments, w is 2.

In some embodiments, w is 3.

In some embodiments, the compound is of Formula I, or a pharmaceutically acceptable salt or solvate thereof, wherein A is A-1. In some embodiments, M¹ is 4- to 8-membered heterocyclenyl. In some embodiments, M¹ is phenylenyl. In some embodiments, M¹ is 5-membered heteroarylenyl. In some embodiments, M¹ is 6-membered heteroarylenyl.

In some embodiments, the compound is of Formula I, or a pharmaceutically acceptable salt or solvate thereof, wherein A is A-1 and M¹ is selected from:

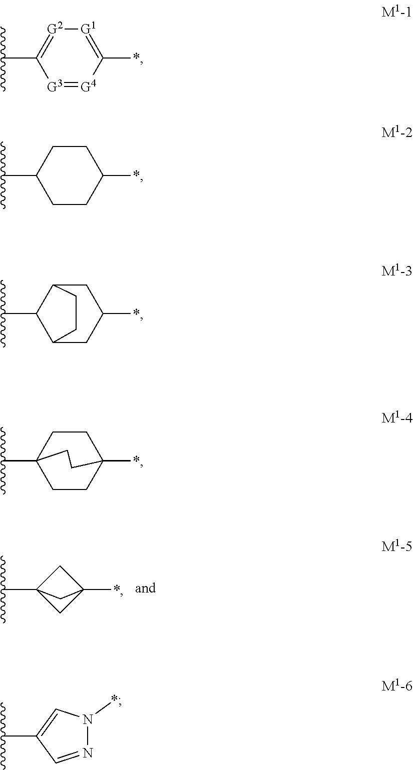

wherein the bond designated with an “*” is attached to E¹;
G¹ is selected from —N═ and —CR^{3a}═;
G² is selected from —N═ and —CR^{3b}═;
G³ is selected from —N═ and —CR^{3c}═;
G⁴ is selected from —N═ and —CR^{3d}═; and
R^{3a}, R^{3b}, R^{3c}, R^{3d} are independently selected from hydrogen and halo,
with the proviso that E¹ is —(CH₂)_{b}— when M¹ is M¹-6.

In some embodiments, the compound is of Formula I, or a pharmaceutically acceptable salt or solvate thereof, wherein A is A-1 and M¹ is M¹-1. In some embodiments, M¹ is M¹-2. In some embodiments, M¹ is M¹-3. In some embodiments, M¹ is M¹-4. In some embodiments, M¹ is M¹-5. In some embodiments, M¹ is M¹-6.

In some embodiments, the compound is of Formula I, or a pharmaceutically acceptable salt or solvate thereof, wherein A is A-2. In some embodiments, M² is 4- to 8-membered heterocyclenyl. In some embodiments, M² is phenylenyl. In some embodiments, M² is 5-membered heteroarylenyl. In some embodiments, M² is 6-membered heteroarylenyl.

In some embodiments, the compound is of Formula I, or a pharmaceutically acceptable salt or solvate thereof, wherein A is A-2 and M² is selected from:

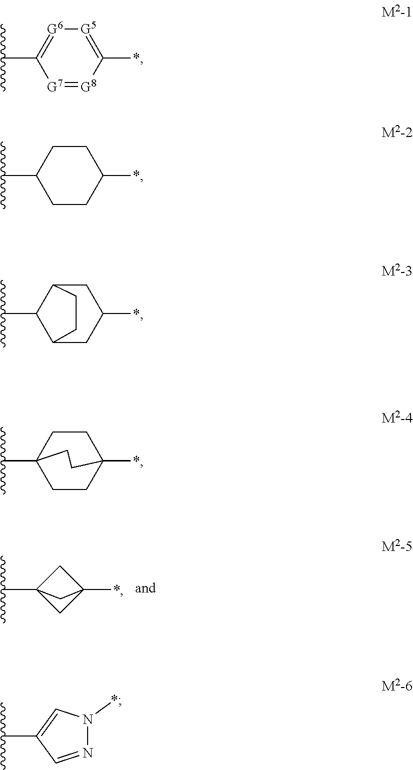

wherein the bond designated with an “*” is attached to E²;
G⁵ is selected from —N═ and —CR^{7a}═;
G⁶ is selected from —N═ and —CR^{7b}═;
G⁷ is selected from —N═ and —CR^{7c}═;
G⁸ is selected from —N═ and —CR^{7d}═; and
R^{7a}, R^{7b}, R^{7c}, and R^{7d} are independently selected from hydrogen and halo,
with the proviso that E² is —(CH₂)_{c}— when M² is M²-6.

In some embodiments, the compound is of Formula I, or a pharmaceutically acceptable salt or solvate thereof, wherein A is A-2 and M² is M²-1. In some embodiments, M² is M²-2. In some embodiments, M² is M²-3. In some embodiments, M² is M²-4. In some embodiments, M² is M²-5. In some embodiments, M² is M²-6.

In some embodiments, the compound is of Formula I, or a pharmaceutically acceptable salt or solvate thereof, wherein A is A-3. In some embodiments, M³ is 4- to 8-membered heterocyclenyl. In some embodiments, M³ is phenylenyl. In some embodiments, M³ is 5-membered heteroarylenyl. In some embodiments, M³ is 6-membered heteroarylenyl.

In some embodiments, the compound is of Formula I, or a pharmaceutically acceptable salt or solvate thereof, wherein A is A-3 and M³ is selected from:

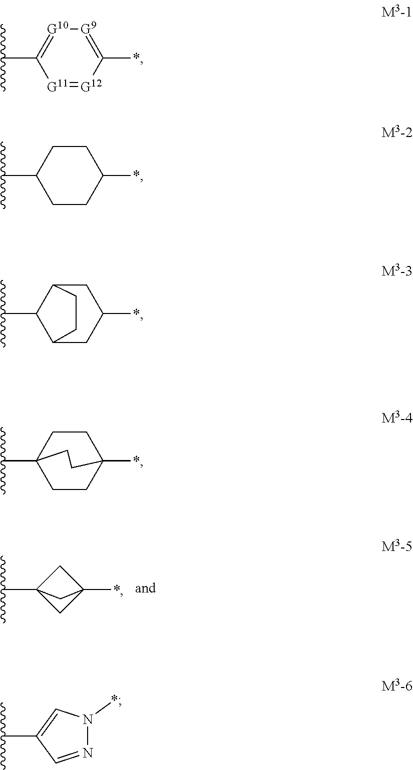

wherein the bond designated with an “*” is attached to E³;
G⁹ is selected from —N═ and —CR^{11a}═;
G¹⁰ is selected from —N═ and —CR^{11b}═;
G¹¹ is selected from —N═ and —CR^{11c}═;
G¹² is selected from —N═ and —CR^{11d}═; and
R^{11a}, R^{11b}, R^{11c}, and R^{11d} are independently selected from hydrogen and halo,
with the proviso that E³ is —(CH₂)_{d}— when M² is M³-6.

In some embodiments, the compound is of Formula I, or a pharmaceutically acceptable salt or solvate thereof, wherein A is A-3 and M³ is M³-1. In some embodiments, M³ is M³-2.

In some embodiments, M³ is M³-3. In some embodiments, M³ is M³-4. In some embodiments, M³ is M³-5. In some embodiments, M³ is M³-6.

In some embodiments, the compound is of Formula I, or a pharmaceutically acceptable salt or solvate thereof, wherein A is A-4.

In some embodiments, the compound is of Formula I, or a pharmaceutically acceptable salt or solvate thereof, wherein A is A-5.

In some embodiments, the compound is of Formula II:

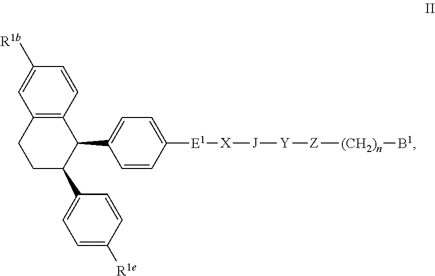

or a pharmaceutically acceptable salt or solvate thereof, wherein R^{1e} is selected from hydrogen and halo; and R^{1b}, E¹, X, J, Y, Z, n, and B¹ are as described herein.

In some embodiments, the compound is of Formula III:

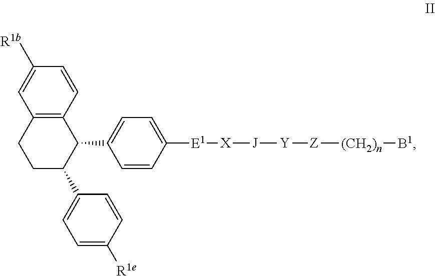

or a pharmaceutically acceptable salt or solvate thereof, wherein R^{1e} is selected from hydrogen and halo; and R^{1b}, E¹, X, J, Y, Z, n, and B¹ are as described herein.

In some embodiments, the compound is of any one of Formulae I-III, or a pharmaceutically acceptable salt or solvate thereof, wherein R^{1b} is hydroxy and R^{1e} is hydrogen or fluoro. In some embodiments, R^{1e} is hydrogen.

In some embodiments, the compound is of any one of Formulae I-III, or a pharmaceutically acceptable salt or solvate thereof, wherein E¹ is —O—.

In some embodiments, the compound is of any one of Formulae I-III, or a pharmaceutically acceptable salt or solvate thereof, wherein E¹ is —(CH₂)_{b}—. In some embodiments, b is 0.

In some embodiments, the compound is of Formula IV:

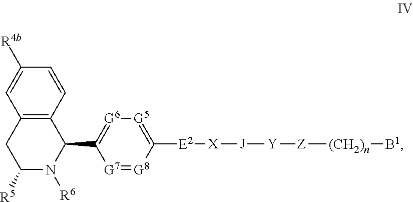

or a pharmaceutically acceptable salt or solvate thereof, wherein R^{4b}, R⁵, R⁶, G⁵, G⁶, G⁷, G⁸, E², X, J, Y, Z, n, and B¹ are as described herein.

In some embodiments, the compound is of Formula IV, or a pharmaceutically acceptable salt or solvate thereof, wherein R^{4b} is hydroxy. In some embodiments, R⁵ is methyl.

In some embodiments, the compound is of Formula V:

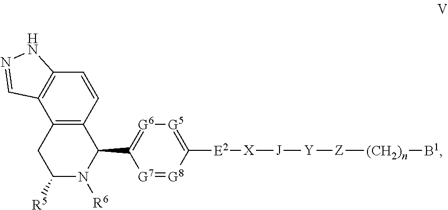

or a pharmaceutically acceptable salt or solvate thereof, wherein R⁵, R⁶, G⁵, G⁶, G⁷, G⁸, E², X, J, Y, Z, n, and B¹ are as described herein.

In some embodiments, the compound is of Formulae IV or V, or a pharmaceutically acceptable salt or solvate thereof, wherein R⁶ is selected from —CH₂CF₂CH₃, —CH₂CF₂H, and —CH₂CF₃. In some embodiments, R⁶ is —CH₂CF₂CH₃. In some embodiments, R⁶ is —CH₂CF₂H. In some embodiments, R⁶ is —CH₂CF₃.

In some embodiments, the compound is of Formulae IV or V, or a pharmaceutically acceptable salt or solvate thereof, wherein G⁵ is —N═.

In some embodiments, the compound is of Formulae IV or V, or a pharmaceutically acceptable salt or solvate thereof, wherein G⁵ is —CR^{7a}═. In some embodiments, R^{7a} is selected from hydrogen and fluoro. In some embodiments, R^{7a} is hydrogen. In some embodiments, R^{7a} is fluroro.

In some embodiments, the compound is of Formulae IV or V, or a pharmaceutically acceptable salt or solvate thereof, wherein G⁶ is —N═.

In some embodiments, the compound is of Formulae IV or V, or a pharmaceutically acceptable salt or solvate thereof, wherein G⁶ is —CR^{7b}═. In some embodiments, R^{7b} is selected from hydrogen and fluoro. In some embodiments, R^{7b} is hydrogen. In some embodiments, R^{7b} is fluroro.

In some embodiments, the compound is of Formulae IV or V, or a pharmaceutically acceptable salt or solvate thereof, wherein G⁷ is —N═.

In some embodiments, the compound is of Formulae IV or V, or a pharmaceutically acceptable salt or solvate thereof, wherein G⁷ is —CR^{7c}═. In some embodiments, R^{7c} is selected from hydrogen and fluoro. In some embodiments, R^{7c} is hydrogen. In some embodiments, R^{7c} is fluroro.

In some embodiments, the compound is of Formulae IV or V, or a pharmaceutically acceptable salt or solvate thereof, wherein G⁸ is —N═.

In some embodiments, the compound is of Formulae IV or V, or a pharmaceutically acceptable salt or solvate thereof, wherein G⁸ is —CR^{7d}═. In some embodiments, R^{7d} is selected from hydrogen and fluoro. In some embodiments, R^{7d} is hydrogen. In some embodiments, R^{7d} is fluroro.

In some embodiments, the compound is of Formulae IV or V, or a pharmaceutically acceptable salt or solvate thereof, wherein E² is —(CH₂)_{c}—. In some embodiments, c is 0.

In some embodiments, the compound is of Formula VI:

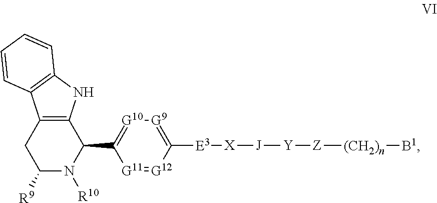

or a pharmaceutically acceptable salt or solvate thereof, wherein R⁹, R¹⁰, G⁹, G¹⁰, G¹¹, G¹², E³, X, J, Y, Z, n, and B¹ are as described herein.

In some embodiments, the compound is of Formula VI, or a pharmaceutically acceptable salt or solvate thereof, wherein R⁹ is methyl.

In some embodiments, the compound is of Formula VI, or a pharmaceutically acceptable salt or solvate thereof, wherein R¹⁰ is selected from —CH₂CF₂CH₃, —CH₂CF₂H, and —CH₂CF₃. In some embodiments, R¹⁰ is —CH₂CF₂CH₃. In some embodiments, R¹⁰ is —CH₂CF₂H. In some embodiments, R¹⁰ is —CH₂CF₃

In some embodiments, the compound is of Formula VI, or a pharmaceutically acceptable salt or solvate thereof, wherein G⁹ is —N═.

In some embodiments, the compound is of Formula VI, or a pharmaceutically acceptable salt or solvate thereof, wherein G⁹ is —CR^{11a}═. In some embodiments, R^{11a} is selected from hydrogen and fluoro. In some embodiments, R^{11a} is hydrogen. In some embodiments, R^{11a} is fluroro.

In some embodiments, the compound is of Formula VI, or a pharmaceutically acceptable salt or solvate thereof, wherein G¹⁰ is —N═.

In some embodiments, the compound is of Formula VI, or a pharmaceutically acceptable salt or solvate thereof, wherein G¹⁰ is —CR^{11b}═. In some embodiments, R^{11b} is selected from hydrogen and fluoro. In some embodiments, R^{11b} is hydrogen. In some embodiments, R^{11b} is fluroro.

In some embodiments, the compound is of Formula VI, or a pharmaceutically acceptable salt or solvate thereof, wherein G¹¹ is —N═.

In some embodiments, the compound is of Formula VI, or a pharmaceutically acceptable salt or solvate thereof, wherein G¹¹ is —CR^{11c}═. In some embodiments, R^{11c} is selected from hydrogen and fluoro. In some embodiments, R^{11c} is hydrogen. In some embodiments, R^{11c} is fluroro.

In some embodiments, the compound is of Formula VI, or a pharmaceutically acceptable salt or solvate thereof, wherein G¹² is —N═.

In some embodiments, the compound is of Formula VI, or a pharmaceutically acceptable salt or solvate thereof, wherein G¹² is —CR^{11d}═. In some embodiments, R^{11d} is selected from hydrogen and fluoro. In some embodiments, R^{11d} is hydrogen. In some embodiments, R^{11d} is fluroro.

In some embodiments, the compound is of Formula VI, or a pharmaceutically acceptable salt or solvate thereof, wherein E³ is —(CH₂)_{d}—. In some embodiments, d is 0.

In some embodiments, the compound is of Formula VII:

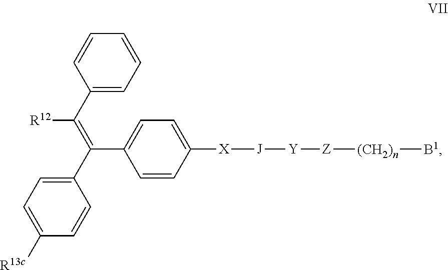

or a pharmaceutically acceptable salt or solvate thereof, wherein R¹², R^{13c}, X, J, Y, Z, n, and B¹ are as described herein.

In some embodiments, the compound is of Formula VII, or a pharmaceutically acceptable salt or solvate thereof, wherein R¹² is selected from —CH₂CH₃ and —CH₂CH₂Cl.

In some embodiments, the compound is of Formula VII, or a pharmaceutically acceptable salt or solvate thereof, wherein R^{13c} is hydroxy.

In some embodiments, the compound is of Formula VIII:

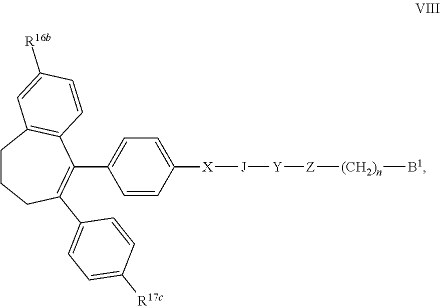

or a pharmaceutically acceptable salt or solvate thereof, wherein R^{16b}, R^{17c}, X, J, Y, Z, n, and B¹ are as described herein.

In some embodiments, the compound is of Formula VIII, or a pharmaceutically acceptable salt or solvate thereof, wherein R^{16b} is hydroxy.

In some embodiments, the compound is of Formula VIII, or a pharmaceutically acceptable salt or solvate thereof, wherein R^{17c} is fluoro.

In some embodiments, the compound is of any one of Formulae I-VIII, or a pharmaceutically acceptable salt or solvate thereof, wherein X is cycloalkylenyl. In some embodiments, X is selected from:

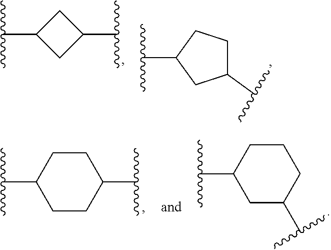

In some embodiments, the compound is of any one of Formulae I-VIII, or a pharmaceutically acceptable salt or solvate thereof, wherein X is heterocyclenyl.

In some embodiments, the compound is of any one of Formulae I-VIII, or a pharmaceutically acceptable salt or solvate thereof, wherein X is optionally substituted 4- to 8-membered heterocyclenyl. In some embodiments, X is selected from:

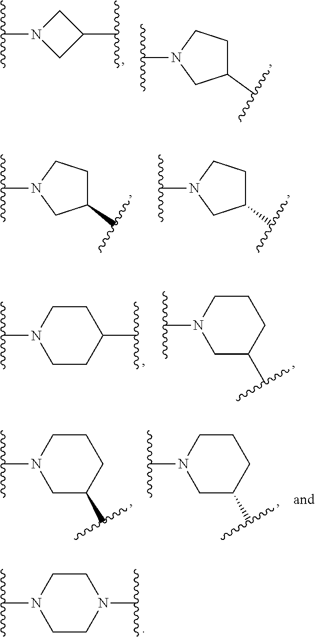

In some embodiments, the compound is of any one of Formulae I-VIII, or a pharmaceutically acceptable salt or solvate thereof, wherein X is a 7- to 14-membered spiroheterocyclenyl.

In some embodiments, the compound is of any one of Formulae I-VIII, or a pharmaceutically acceptable salt or solvate thereof, wherein X is:

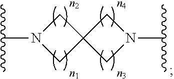

and
n₁, n₂, n₃, and n₄ are independently 0, 1, 2, 3, or 4, with the proviso that the sum of n₁, n₂, n₃, and n₄ is 4, 5, 6, 7, 8, 9, or 10.

In some embodiments, the compound is of any one of Formulae I-VIII, or a pharmaceutically acceptable salt or solvate thereof, wherein X is selected from:

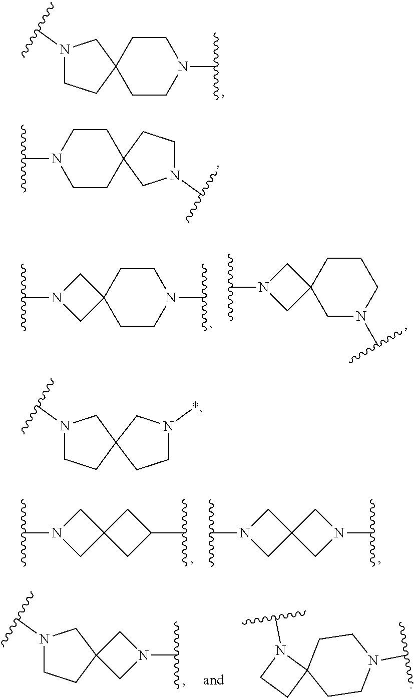

In some embodiments, the compound is of any one of Formulae I-VIII, or a pharmaceutically acceptable salt or solvate thereof, wherein J is —(CH₂)ₘ— and m is 0 or 1. In some embodiments, m is 0. In some embodiments, m is 1.

In some embodiments, the compound is of any one of Formulae I-VIII, or a pharmaceutically acceptable salt or solvate thereof, wherein J is —(CH₂)_{z1}N(R¹⁹)— and z1 is 0 or 1. In some embodiments, z1 is 0. In some embodiments, z1 is 1. In some embodiments, R¹⁹ is hydrogen or methyl.

In some embodiments, the compound is of any one of Formulae I-VIII, or a pharmaceutically acceptable salt or solvate thereof, wherein J is —(CH₂)_{z2}O— and z2 is 0 or 1.

In some embodiments, z2 is 0. In some embodiments, z2 is 1.

In some embodiments, the compound is of any one of Formulae I-VIII, or a pharmaceutically acceptable salt or solvate thereof, wherein Y is selected from heterocyclenyl, —C(═O)— and —(CH₂)ᵣ—. In some embodiments, Y is optionally substituted 4- to 8-membered heterocyclenyl. In some embodiments, Y is:

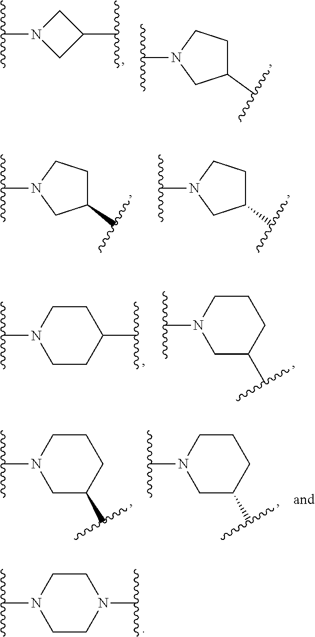

In some embodiments, Y is —(CH₂)ᵣ—. In some embodiments, r is 0. In some embodiments, r is 1. In some embodiments, Y is —C(═O)—.

In some embodiments, the compound is of any one of Formulae I-VIII, or a pharmaceutically acceptable salt or solvate thereof, wherein Z is selected from heterocyclenyl and —(CH₂)ₛ—. In some embodiments, Z is optionally substituted 4- to 8-membered heterocyclenyl. In some embodiments, Z is:

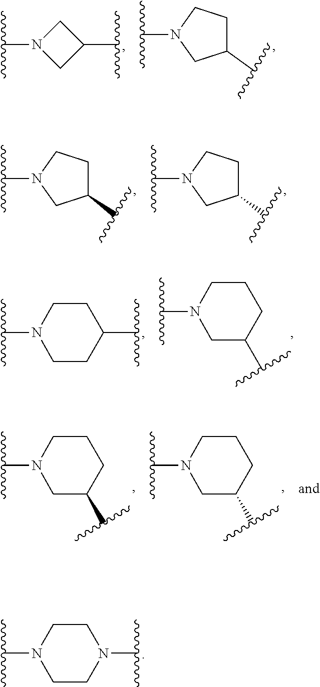

In some embodiments, Z is —(CH₂)ₛ—. In some embodiments, s is 0. In some embodiments, s is 1. In some embodiments, Z is —C(═O)—.

In some embodiments, the compound is of any one of Formulae I-VIII, or a pharmaceutically acceptable salt or solvate thereof, wherein n is 0 or 1. In some embodiments, n is 0. In some embodiments, n is 1.

### Compounds of the Disclosure

In some aspects, the disclosure provides compounds of any one of Formulae I-VIII, or a pharmaceutically acceptable salt or solvate thereof, wherein B¹ is B¹-1, B¹-2, B¹-3, B¹-4, B¹-5, B¹-6, or B¹-7. These compounds, or a pharmaceutically acceptable salt or solvate thereof, are collectively referred to as “Compounds of the Disclosure.”

In some embodiments, Compounds of the Disclosure are compounds of any one of Formulae I-VIII, or a pharmaceutically acceptable salt or solvate thereof, wherein B¹ is B¹-1. In some embodiments, B¹ is B¹-1-B:

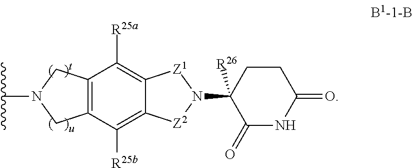

In some embodiments, B¹ is B¹-1-C:

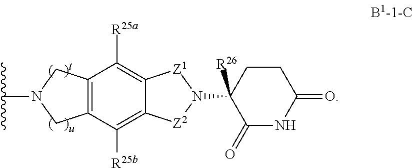

In some embodiments, t is 1. In some embodiments, t is 2. In some embodiments, u is 1. In some embodiments, u is 2.

In some embodiments, Compounds of the Disclosure are compounds of any one of Formulae I-VIII, or a pharmaceutically acceptable salt or solvate thereof, wherein B¹ is B¹-2.

In some embodiments, B¹ is B¹-2-B:

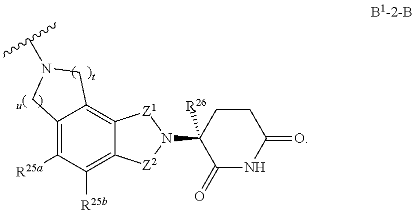

In some embodiments, B¹ is B¹-2-C:

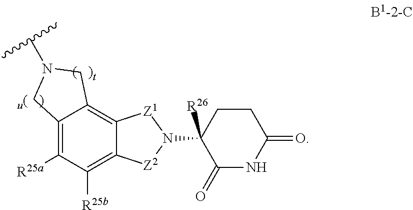

In some embodiments, t is 1. In some embodiments, t is 2. In some embodiments, u is 1. In some embodiments, u is 2.

In some embodiments, Compounds of the Disclosure are compounds of any one of Formulae I-VIII, or a pharmaceutically acceptable salt or solvate thereof, wherein B¹ is B¹-3. In some embodiments, B¹ is B¹-3-B:

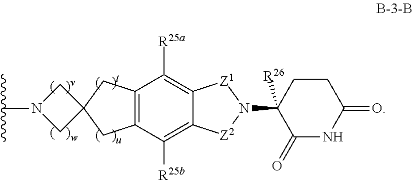

In some embodiments, B¹ is B¹-3-C:

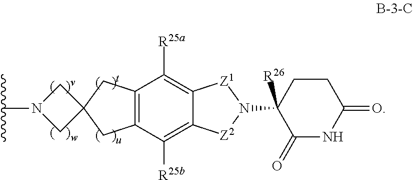

In some embodiments, t is 1. In some embodiments, t is 2. In some embodiments, u is 1. In some embodiments, u is 2. In some embodiments, v is 1. In some embodiments, v is 2. In some embodiments, w is 1. In some embodiments, w is 2.

In some embodiments, Compounds of the Disclosure are compounds of any one of Formulae I-VIII, or a pharmaceutically acceptable salt or solvate thereof, wherein B¹ is B¹-4.

In some embodiments, B¹ is B¹-4-B:

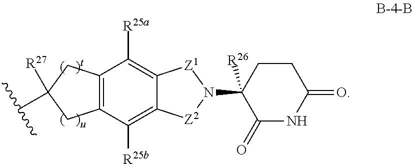

In some embodiments, B¹ is B¹-4-C:

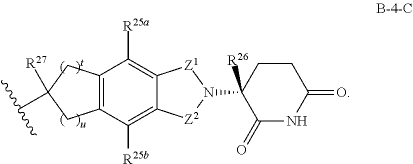

In some embodiments, t is 1. In some embodiments, t is 2. In some embodiments, u is 1. In some embodiments, u is 2. In some embodiments, R²⁷ is hydrogen.

In some embodiments, Compounds of the Disclosure are compounds of any one of Formulae I-VIII, or a pharmaceutically acceptable salt or solvate thereof, wherein B¹ is B¹-5. In some embodiments, B¹ is B¹-5-B:

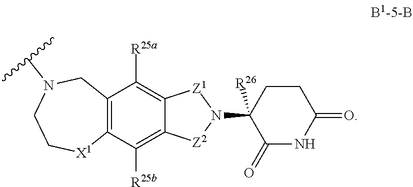

In some embodiments, X¹ is —O—.

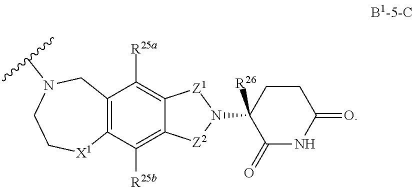

In some embodiments, Compounds of the Disclosure are compounds of any one of Formulae I-VIII, or a pharmaceutically acceptable salt or solvate thereof, wherein B¹ is B¹-6. In some embodiments, B¹ is B¹-6-B:

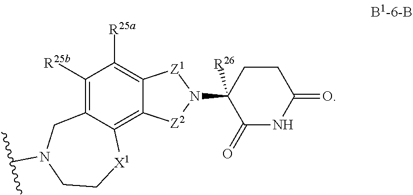

In some embodiments, B¹ is B¹-6-C:

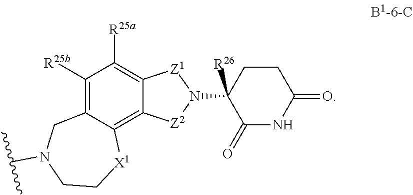

In some embodiments, X¹ is —O—.

In some embodiments, Compounds of the Disclosure are compounds of any one of Formulae I-VIII, or a pharmaceutically acceptable salt or solvate thereof, wherein B¹ is B¹-7.

In some embodiments, B¹ is B¹-7-B:

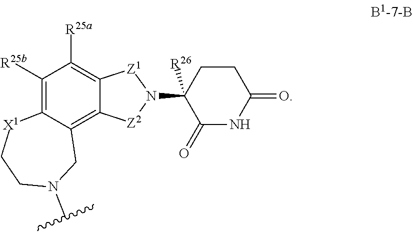

In some embodiments, B¹ is B¹-7-C:

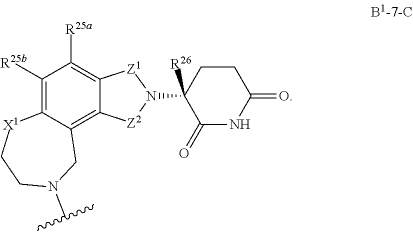

In some embodiments, X¹ is —O—.

In some embodiments, Compounds of the Disclosure are compounds of any one of Formulae I-VIII, or a pharmaceutically acceptable salt or solvate thereof, wherein R^{25a} is hydrogen.

In some embodiments, Compounds of the Disclosure are compounds of any one of Formulae I-VIII, or a pharmaceutically acceptable salt or solvate thereof, wherein R^{25b} is hydrogen.

In some embodiments, Compounds of the Disclosure are compounds of any one of Formulae I-VIII, or a pharmaceutically acceptable salt or solvate thereof, wherein R²⁶ is hydrogen.

In some embodiments, Compounds of the Disclosure of any one of Formulae I-VIII, or a pharmaceutically acceptable salt or solvate thereof, wherein Z¹ is —C(═O)— and Z² is —C(═O)—.

In some embodiments, Compounds of the Disclosure are compounds of any one of Formulae I-VIII, or a pharmaceutically acceptable salt or solvate thereof, wherein Z¹ is —CH₂— and Z² is —C(═O)—.

In some embodiments, Compounds of the Disclosure are compounds of any one of Formulae I-VIII, or a pharmaceutically acceptable salt or solvate thereof, wherein Z¹ is —C(═O)— and Z² is —CH₂—.

In some embodiments, Compounds of the Disclosure are selected from one or more of the compounds of Table 1, and pharmaceutically acceptable salts and solvates thereof.

In some embodiments, Compounds of the Disclosure are selected from one or more of the compounds of Table 1, and pharmaceutically acceptable salts thereof.

In some embodiments, Compounds of the Disclosure are selected from one or more of the compounds of Table 1.

TABLE 1

| Cpd. | | |
|---|---|---|
| No. | Structure | Name |
| E 1 | 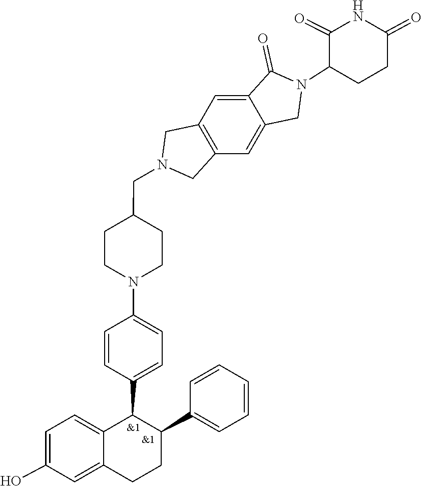 [SMILES: O=C1CCC(N2Cc3cc4c(cc3C2=O)CN(CC2CCN(c3ccc([C@@H]5c6ccc(O)cc6CC[C@@H]5c5ccccc5)cc3)CC2)C4)C(=O)N1] | Rac-3-(6-((1-(4-((1R,2S)-6-hydroxy-2- phenyl-1,2,3,4-tetrahydronaphthalen-1- yl)phenyl)piperidin-4-yl)methyl)-1-oxo- 3,5,6,7-tetrahydropyrrolo[3,4-f]isoindol- 2(1H)-yl)piperidine-2,6-dione |
| E 2 | 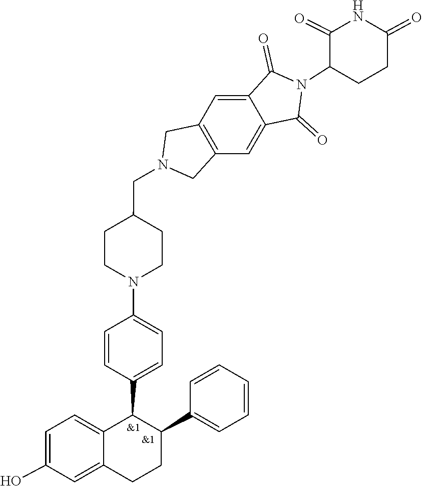 [SMILES: O=C1CCC(N2C(=O)c3cc4c(cc3C2=O)CN(CC2CCN(c3ccc([C@@H]5c6ccc(O)cc6CC[C@@H]5c5ccccc5)cc3)CC2)C4)C(=O)N1] | Rac-2-(2,6-dioxopiperidin-3-yl)-6-((1- (4-((1R,2S)-6-hydroxy-2-phenyl- 1,2,3,4-tetrahydronaphthalen-1- yl)phenyl)piperidin-4-yl)methyl)-6,7- dihydropyrrolo[3,4-f]isoindole- 1,3(2H,5H)-dione |
| E 3 | 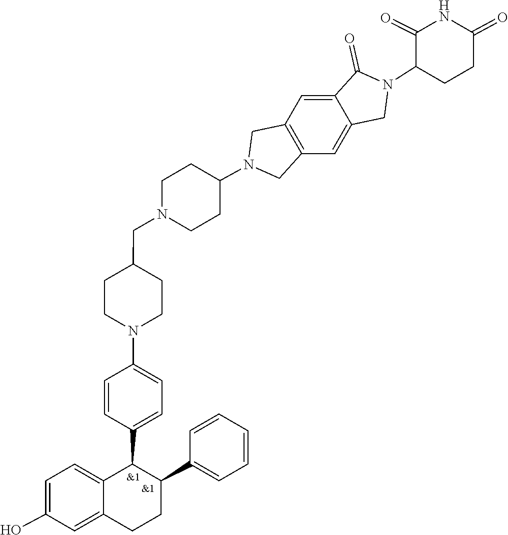 [SMILES: O=C1CCC(N2Cc3cc4c(cc3C2=O)CN(C2CCN(CC3CCN(c5ccc([C@@H]6c7ccc(O)cc7CC[C@@H]6c6ccccc6)cc5)CC3)CC2)C4)C(=O)N1] | Rac-3-(6-(1-((1-(4-((1R,2S)-6-hydroxy- 2-phenyl-1,2,3,4-tetrahydronaphthalen- 1-yl)phenyl)piperidin-4- yl)methyl)piperidin-4-yl)-1-oxo-3,5,6,7- tetrahydropyrrolo[3,4-f]isoindol-2(1H)- yl)piperidine-2,6-dione |
| E 4 | 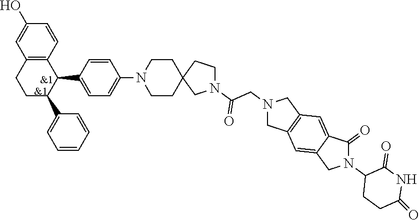 [SMILES: O=C1CCC(N2Cc3cc4c(cc3C2=O)CN(CC(=O)N2CCC3(CCN(c5ccc([C@@H]6c7ccc(O)cc7CC[C@@H]6c6ccccc6)cc5)CC3)C2)C4)C(=O)N1] | rac-3-(6-(2-(8-(4-((1R,2S)-6-hydroxy-2- phenyl-1,2,3,4-tetrahydronaphthalen-1- yl)phenyl)-2,8-diazaspiro[4.5]decan-2- yl)-2-oxoethyl)-1-oxo-3,5,6,7- tetrahydropyrrolo[3,4-f]isoindol-2(1H)- yl)piperidine-2,6-dione |
| E 5 | 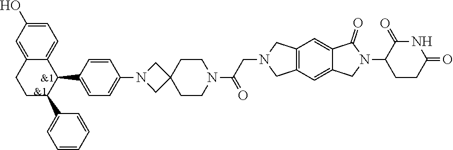 [SMILES: O=C1CCC(N2Cc3cc4c(cc3C2=O)CN(CC(=O)N2CCC3(CC2)CN(c2ccc([C@@H]5c6ccc(O)cc6CC[C@@H]5c5ccccc5)cc2)C3)C4)C(=O)N1] | rac-3-(6-(2-(2-(4-((1R,2S)-6-hydroxy-2- phenyl-1,2,3,4-tetrahydronaphthalen-1- yl)phenyl)-2,7-diazaspiro[3.5]nonan-7- yl)-2-oxoethyl)-1-oxo-3,5,6,7- tetrahydropyrrolo[3,4-f]isoindol-2(1H)- yl)piperidine-2,6-dione |
| E 6 | 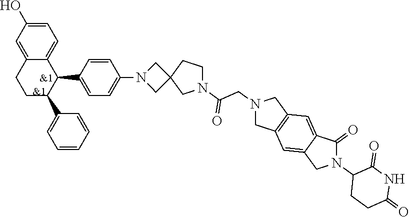 [SMILES: O=C1CCC(N2Cc3cc4c(cc3C2=O)CN(CC(=O)N2CCC3(C2)CN(c2ccc([C@@H]5c6ccc(O)cc6CC[C@@H]5c5ccccc5)cc2)C3)C4)C(=O)N1] | rac-3-(6-(2-(2-(4-((1R,2S)-6-hydroxy-2- phenyl-1,2,3,4-tetrahydronaphthalen-1- yl)phenyl)-2,6-diazaspiro[3.4]octan-6- yl)-2-oxoethyl)-1-oxo-3,5,6,7- tetrahydropyrrolo[3,4-f]isoindol-2(1H)- yl)piperidine-2,6-dione |
| E 7 | 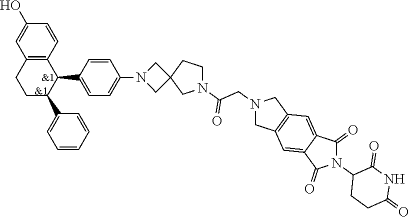 [SMILES: O=C1CCC(N2C(=O)c3cc4c(cc3C2=O)CN(CC(=O)N2CCC3(C2)CN(c2ccc([C@@H]5c6ccc(O)cc6CC[C@@H]5c5ccccc5)cc2)C3)C4)C(=O)N1] | rac-2-(2,6-dioxopiperidin-3-yl)-6-(2-(8- (4-((1R,2S)-6-hydroxy-2-phenyl- 1,2,3,4-tetrahydronaphthalen-1- yl)phenyl)-2,8-diazaspiro[4.5]decan-2- yl)-2-oxoethyl)-6,7-dihydropyrrolo[3,4- f]isoindole-1,3(2H,5H)-dione |
| E 8 | 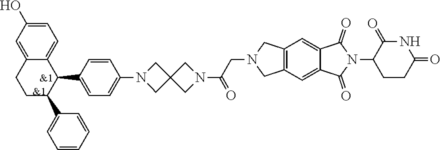 [SMILES: O=C1CCC(N2C(=O)c3cc4c(cc3C2=O)CN(CC(=O)N2CC3(C2)CN(c2ccc([C@@H]5c6ccc(O)cc6CC[C@@H]5c5ccccc5)cc2)C3)C4)C(=O)N1] | Rac-2-(2,6-dioxopiperidin-3-yl)-6-(2-(6- (4-((1R,2S)-6-hydroxy-2-phenyl- 1,2,3,4-tetrahydronaphthalen-1- yl)phenyl)-2,6-diazaspiro[3.3]heptan-2- yl)-2-oxoethyl)-6,7-dihydropyrrolo[3,4- f]isoindole-1,3(2H,5H)-dione |
| E 9 | 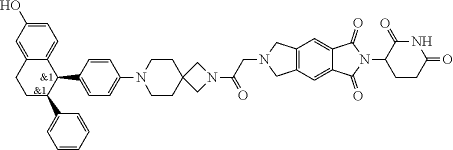 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)C4)C(=O)N1] | Rac-2-(2,6-dioxopiperidin-3-yl)-6-(2-(7- (4-((1R,2S)-6-hydroxy-2-phenyl- 1,2,3,4-tetrahydronaphthalen-1- yl)phenyl)-2,7-diazaspiro[3.5]nonan-2- yl)-2-oxoethyl)-6,7-dihydropyrrolo[3,4- f]isoindole-1,3(2H,5H)-dione |
| E 10 | 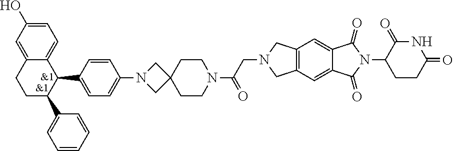 [SMILES: O=C1CCC(N2C(=O)c3cc4c(cc3C2=O)CN(CC(=O)N2CCC3(CC2)CN(c2ccc([C@@H]5c6ccc(O)cc6CC[C@@H]5c5ccccc5)cc2)C3)C4)C(=O)N1] | Rac-2-(2,6-dioxopiperidin-3-yl)-6-(2-(2- (4-((1R,2S)-6-hydroxy-2-phenyl- 1,2,3,4-tetrahydronaphthalen-1- yl)phenyl)-2,7-diazaspiro[3.5]nonan-7- yl)-2-oxoethyl)-6,7-dihydropyrrolo[3,4- f]isoindole-1,3(2H,5H)-dione |
| E 11 | 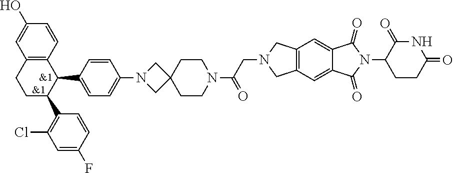 [SMILES: O=C1CCC(N2C(=O)c3cc4c(cc3C2=O)CN(CC(=O)N2CCC3(CC2)CN(c2ccc([C@@H]5c6ccc(O)cc6CC[C@@H]5c5ccc(F)cc5Cl)cc2)C3)C4)C(=O)N1] | rac-6-(2-(2-(4-((1R,2S)-2-(2-chloro-4- fluorophenyl)-6-hydroxy-1,2,3,4- tetrahydronaphthalen-1-yl)phenyl)-2,7- diazaspiro[3.5]nonan-7-yl)-2-oxoethyl)- 2-(2,6-dioxopiperidin-3-yl)-6,7- dihydropyrrolo[3,4-f]isoindole- 1,3(2H,5H)-dione |
| E 12 | 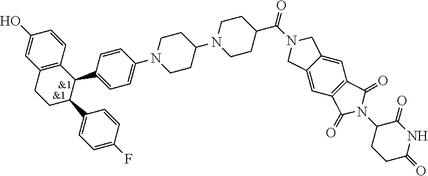 [SMILES: O=C1CCC(N2C(=O)c3cc4c(cc3C2=O)CN(C(=O)C2CCN(C3CCN(c5ccc([C@@H]6c7ccc(O)cc7CC[C@@H]6c6ccc(F)cc6)cc5)CC3)CC2)C4)C(=O)N1] | Rac-2-(2,6-dioxopiperidin-3-yl)-6-(1′- (4-((1R,2S)-2-(4-fluorophenyl)-6- hydroxy-1,2,3,4-tetrahydronaphthalen- 1-yl)phenyl)-[1,4′-bipiperidine]-4- carbonyl)-6,7-dihydropyrrolo[3,4- f]isoindole-1,3(2H,5H)-dione |
| E 13 | 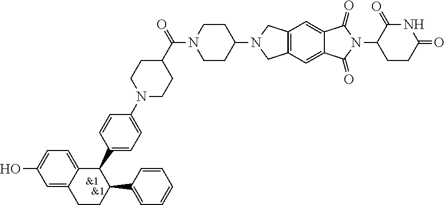 [SMILES: O=C1CCC(N2C(=O)c3cc4c(cc3C2=O)CN(C2CCN(C(=O)C3CCN(c5ccc([C@@H]6c7ccc(O)cc7CC[C@@H]6c6ccccc6)cc5)CC3)CC2)C4)C(=O)N1] | Rac-2-(2,6-dioxopiperidin-3-yl)-6-(1-(1- (4-((1R,2S)-6-hydroxy-2-phenyl- 1,2,3,4-tetrahydronaphthalen-1- yl)phenyl)piperidine-4-carbonyl)pi570 peridin-4-yl)-6,7-dihydropyrrolo[3,4- f]isoindole-1,3(2H,5H)-dione |
| E 14 | 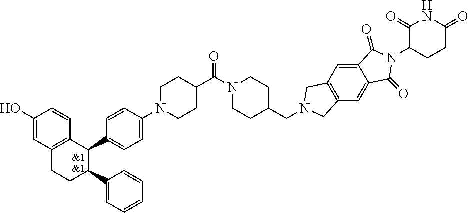 [SMILES: O=C1CCC(N2C(=O)c3cc4c(cc3C2=O)CN(CC2CCN(C(=O)C3CCN(c5ccc([C@@H]6c7ccc(O)cc7CC[C@@H]6c6ccccc6)cc5)CC3)CC2)C4)C(=O)N1] | Rac-2-(2,6-dioxopiperidin-3-yl)-6-((1- (1-(4-((1R,2S)-6-hydroxy-2-phenyl- 1,2,3,4-tetrahydronaphthalen-1- yl)phenyl)piperidine-4- carbonyl)piperidin-4-yl)methyl)-6,7- dihydropyrrolo[3,4-f]isoindole- 1,3(2H,5H)-dione |
| E 15 | 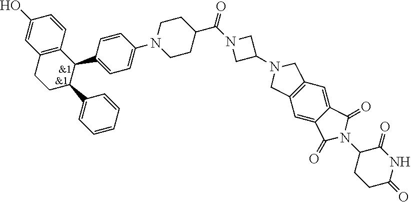 [SMILES: O=C1CCC(N2C(=O)c3cc4c(cc3C2=O)CN(C2CN(C(=O)C3CCN(c5ccc([C@@H]6c7ccc(O)cc7CC[C@@H]6c6ccccc6)cc5)CC3)C2)C4)C(=O)N1] | Rac-2-(2,6-dioxopiperidin-3-yl)-6-(1-(1- (4-((1R,2S)-6-hydroxy-2-phenyl- 1,2,3,4-tetrahydronaphthalen-1- yl)phenyl)piperidine-4- carbonyl)azetidin-3-yl)-6,7- dihydropyrrolo[3,4-f]isoindole- 1,3(2H,5H)-dione |
| E 16 | 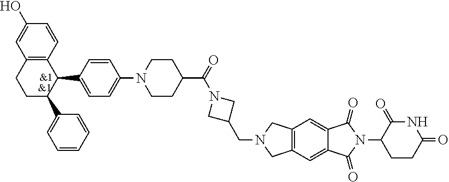 [SMILES: O=C1CCC(N2C(=O)c3cc4c(cc3C2=O)CN(CC2CN(C(=O)C3CCN(c5ccc([C@@H]6c7ccc(O)cc7CC[C@@H]6c6ccccc6)cc5)CC3)C2)C4)C(=O)N1] | Rac-2-(2,6-dioxopiperidin-3-yl)-6-((1- (1-(4-((1R,2S)-6-hydroxy-2-phenyl- 1,2,3,4-tetrahydronaphthalen-1- yl)phenyl)piperidine-4- carbonyl)azetidin-3-yl)methyl)-6,7- dihydropyrrolo[3,4-f]isoindole- 1,3(2H,5H)-dione |
| E 17 | 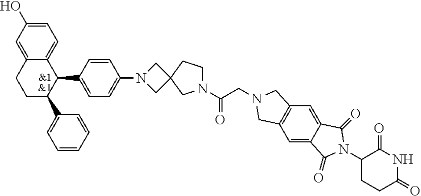 [SMILES: O=C1CCC(N2C(=O)c3cc4c(cc3C2=O)CN(CC(=O)N2CCC3(C2)CN(c2ccc([C@@H]5c6ccc(O)cc6CC[C@@H]5c5ccccc5)cc2)C3)C4)C(=O)N1] | rac-2-(2,6-dioxopiperidin-3-yl)-6-(2-(2- (4-((1R,2S)-6-hydroxy-2-phenyl- 1,2,3,4-tetrahydronaphthalen-1- yl)phenyl)-2,6-diazaspiro[3.4]octan-6- yl)-2-oxoethyl)-6,7-dihydropyrrolo[3,4- f]isoindole-1,3(2H,5H)-dione |
| E 18 | 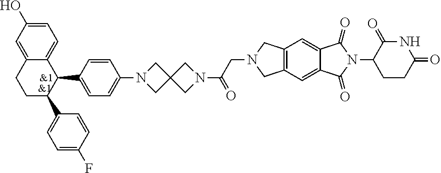 [SMILES: O=C1CCC(N2C(=O)c3cc4c(cc3C2=O)CN(CC(=O)N2CC3(C2)CN(c2ccc([C@@H]5c6ccc(O)cc6CC[C@@H]5c5ccc(F)cc5)cc2)C3)C4)C(=O)N1] | Rac-2-(2,6-dioxopiperidin-3-yl)-6-(2-(6- (4-((1R,2S)-2-(4-fluorophenyl)-6- hydroxy-1,2,3,4-tetrahydronaphthalen- 1-yl)phenyl)-2,6-diazaspiro[3.3]heptan- 2-yl)-2-oxoethyl)-6,7- dihydropyrrolo[3,4-f]isoindole- 1,3(2H,5H)-dione |
| E 19 | 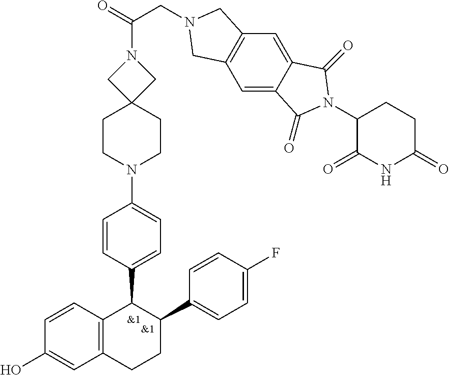 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccc(F)cc6)cc5)CC3)C2)C4)C(=O)N1] | Rac-2-(2,6-dioxopiperidin-3-yl)-6-(2-(7- (4-((1R,2S)-2-(4-fluorophenyl)-6- hydroxy-1,2,3,4-tetrahydronaphthalen- 1-yl)phenyl)-2,7-diazaspiro[3.5]nonan- 2-yl)-2-oxoethyl)-6,7- dihydropyrrolo[3,4-f]isoindole- 1,3(2H,5H)-dione |
| E 20 | 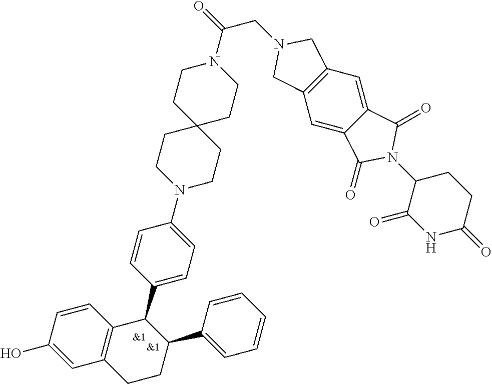 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | Rac-2-(2,6-dioxopiperidin-3-yl)-6-(2-(9- (4-((1R,2S)-6-hydroxy-2-phenyl- 1,2,3,4-tetrahydronaphthalen-1- yl)phenyl)-3,9-diazaspiro[5.5]undecan- 3-yl)-2-oxoethyl)-6,7- dihydropyrrolo[3,4-f]isoindole- 1,3(2H,5H)-dione |
| E 21 | 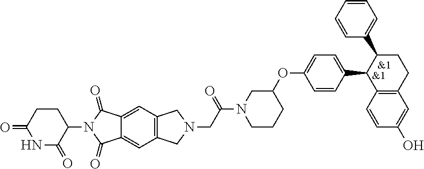 [SMILES: O=C1CCC(N2C(=O)c3cc4c(cc3C2=O)CN(CC(=O)N2CCCC(Oc3ccc([C@@H]5c6ccc(O)cc6CC[C@@H]5c5ccccc5)cc3)C2)C4)C(=O)N1] | Rac-2-(2,6-dioxopiperidin-3-yl)-6-(2-(3- (4-((1R,2S)-6-hydroxy-2-phenyl- 1,2,3,4-tetrahydronaphthalen-1- yl)phenoxy)piperidin-1-yl)-2-oxoethyl)- 6,7-dihydropyrrolo[3,4-f]isoindole- 1,3(2H,5H)-dione |
| E 22 | 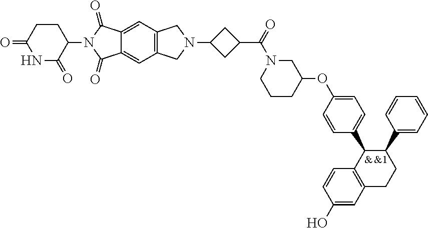 [SMILES: O=C1CCC(N2C(=O)c3cc4c(cc3C2=O)CN(C2CC(C(=O)N3CCCC(Oc5ccc([C@@H]6c7ccc(O)cc7CC[C@@H]6c6ccccc6)cc5)C3)C2)C4)C(=O)N1] | Rac-2-(2,6-dioxopiperidin-3-yl)-6-(3-(3- (4-((1R,2S)-6-hydroxy-2-phenyl- 1,2,3,4-tetrahydronaphthalen-1- yl)phenoxy)piperidine-1- carbonyl)cyclobutyl)-6,7- dihydropyrrolo[3,4-f]isoindole- 1,3(2H,5H)-dione |
| E 23 | 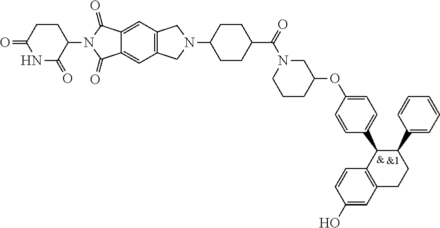 [SMILES: O=C1CCC(N2C(=O)c3cc4c(cc3C2=O)CN(C2CCC(C(=O)N3CCCC(Oc5ccc([C@@H]6c7ccc(O)cc7CC[C@@H]6c6ccccc6)cc5)C3)CC2)C4)C(=O)N1] | Rac-2-(2,6-dioxopiperidin-3-yl)-6-(4-(3- (4-((1R,2S)-6-hydroxy-2-phenyl- 1,2,3,4-tetrahydronaphthalen-1- yl)phenoxy)piperidine-1- carbonyl)cyclohexyl)-6,7- dihydropyrrolo[3,4-f]isoindole- 1,3(2H,5H)-dione |
| E 24 | 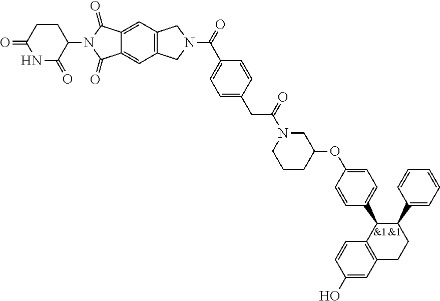 [SMILES: O=C1CCC(N2C(=O)c3cc4c(cc3C2=O)CN(C(=O)c2ccc(CC(=O)N3CCCC(Oc5ccc([C@@H]6c7ccc(O)cc7CC[C@@H]6c6ccccc6)cc5)C3)cc2)C4)C(=O)N1] | Rac-2-(2,6-dioxopiperidin-3-yl)-6-(4-(2- (3-(4-((1R,2S)-6-hydroxy-2-phenyl- 1,2,3,4-tetrahydronaphthalen-1- yl)phenoxy)piperidin-1-yl)-2- oxoethyl)benzoyl)-6,7- dihydropyrrolo[3,4-f]isoindole- 1,3(2H,5H)-dione |
| E 25 | 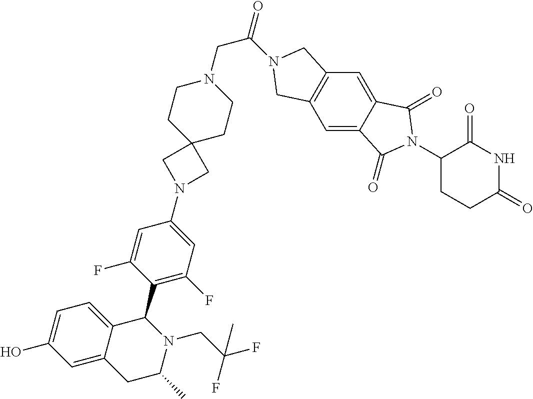 [SMILES: C[C@@H]1Cc2cc(O)ccc2[C@@H](c2c(F)cc(N3CC4(CCN(CC(=O)N5Cc6cc7c(cc6C5)C(=O)N(C5CCC(=O)NC5=O)C7=O)CC4)C3)cc2F)N1CC(C)(F)F] | 6-(2-(2-(4-((1S,3R)-2-(2,2- difluoropropyl)-6-hydroxy-3-methyl- 1,2,3,4-tetrahydroisoquinolin-1-yl)-3,5- difluorophenyl)-2,7- diazaspiro[3.5]nonan-7-yl)acetyl)-2- (2,6-dioxopiperidin-3-yl)-6,7- dihydropyrrolo[3,4-f]isoindole- 1,3(2H,5H)-dione |
| E 26 | 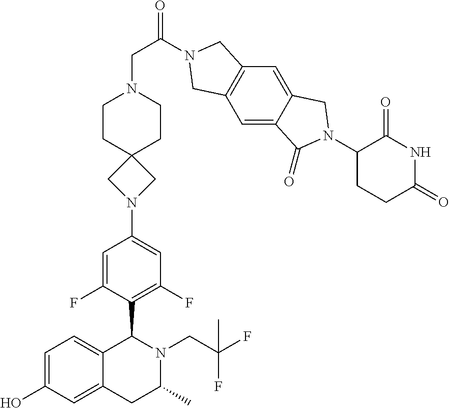 [SMILES: C[C@@H]1Cc2cc(O)ccc2[C@@H](c2c(F)cc(N3CC4(CCN(CC(=O)N5Cc6cc7c(cc6C5)C(=O)N(C5CCC(=O)NC5=O)C7)CC4)C3)cc2F)N1CC(C)(F)F] | 3-(6-(2-(2-(4-((1S,3R)-2-(2,2- difluoropropyl)-6-hydroxy-3-methyl- 1,2,3,4-tetrahydroisoquinolin-1-yl)-3,5- difluorophenyl)-2,7- diazaspiro[3.5]nonan-7-yl)acetyl)-1- oxo-3,5,6,7-tetrahydropyrrolo[3,4- f]isoindol-2(1H)-yl)piperidine-2,6-dione |
| E 27 | 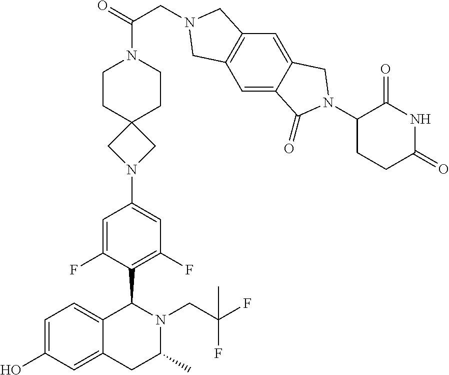 [SMILES: C[C@@H]1Cc2cc(O)ccc2[C@@H](c2c(F)cc(N3CC4(CCN(C(=O)CN5Cc6cc7c(cc6C5)C(=O)N(C5CCC(=O)NC5=O)C7)CC4)C3)cc2F)N1CC(C)(F)F] | 3-(6-(2-(2-(4-((1S,3R)-2-(2,2- difluoropropyl)-6-hydroxy-3-methyl- 1,2,3,4-tetrahydroisoquinolin-1-yl)-3,5- difluorophenyl)-2,7- diazaspiro[3.5]nonan-7-yl)-2-oxoethyl)- 1-oxo-3,5,6,7-tetrahydropyrrolo[3,4- f]isoindol-2(1H)-yl)piperidine-2,6-dione |
| E 28 | 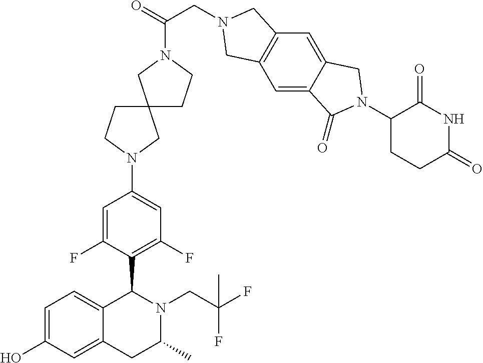 [SMILES: C[C@@H]1Cc2cc(O)ccc2[C@@H](c2c(F)cc(N3CCC4(CCN(C(=O)CN5Cc6cc7c(cc6C5)C(=O)N(C5CCC(=O)NC5=O)C7)C4)C3)cc2F)N1CC(C)(F)F] | 3-(6-(2-(7-(4-((1S,3R)-2-(2,2- difluoropropyl)-6-hydroxy-3-methyl- 1,2,3,4-tetrahydroisoquinolin-1-yl)-3,5- difluorophenyl)-2,7- diazaspiro[4.4]nonan-2-yl)-2-oxoethyl)- 1-oxo-3,5,6,7-tetrahydropyrrolo[3,4- f]isoindol-2(1H)-yl)piperidine-2,6-dione |
| E 29 | 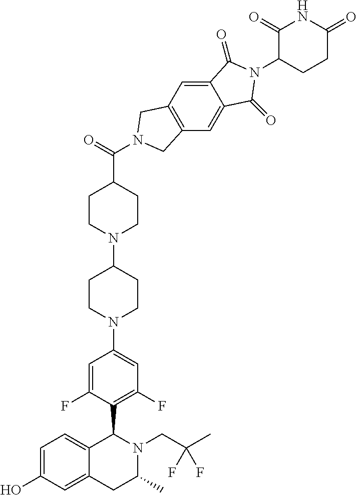 [SMILES: C[C@@H]1Cc2cc(O)ccc2[C@@H](c2c(F)cc(N3CCC(N4CCC(C(=O)N5Cc6cc7c(cc6C5)C(=O)N(C5CCC(=O)NC5=O)C7=O)CC4)CC3)cc2F)N1CC(C)(F)F] | 6-(1′-(4-((1S,3R)-2-(2,2-difluoropropyl)- 6-hydroxy-3-methyl-1,2,3,4- tetrahydroisoquinolin-1-yl)-3,5- difluorophenyl)-[1,4′-bipiperidine]-4- carbonyl)-2-(2,6-dioxopiperidin-3-yl)- 6,7-dihydropyrrolo[3,4-f]isoindole- 1,3(2H,5H)-dione |
| E 30 | 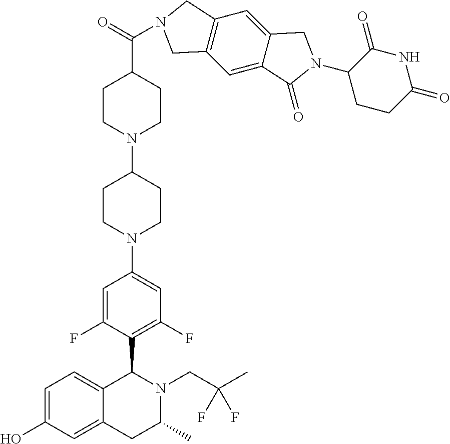 [SMILES: C[C@@H]1Cc2cc(O)ccc2[C@@H](c2c(F)cc(N3CCC(N4CCC(C(=O)N5Cc6cc7c(cc6C5)C(=O)N(C5CCC(=O)NC5=O)C7)CC4)CC3)cc2F)N1CC(C)(F)F] | 3-(6-(1′-(4-((1S,3R)-2-(2,2- difluoropropyl)-6-hydroxy-3-methyl- 1,2,3,4-tetrahydroisoquinolin-1-yl)-3,5- difluorophenyl)-[1,4′-bipiperidine]-4- carbonyl)-1-oxo-3,5,6,7- tetrahydropyrrolo[3,4-f]isoindol-2(1H)- yl)piperidine-2,6-dione |
| E 31 | 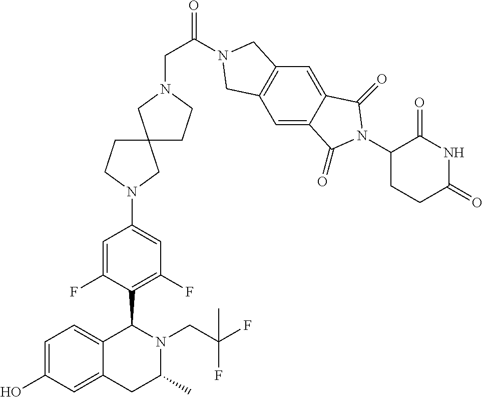 [SMILES: C[C@@H]1Cc2cc(O)ccc2[C@@H](c2c(F)cc(N3CCC4(CCN(CC(=O)N5Cc6cc7c(cc6C5)C(=O)N(C5CCC(=O)NC5=O)C7=O)C4)C3)cc2F)N1CC(C)(F)F] | 6-(2-(7-(4-((1S,3R)-2-(2,2- difluoropropyl)-6-hydroxy-3-methyl- 1,2,3,4-tetrahydroisoquinolin-1-yl)-3,5- difluorophenyl)-2,7- diazaspiro[4.4]nonan-2-yl)acetyl)-2- (2,6-dioxopiperidin-3-yl)-6,7- dihydropyrrolo[3,4-f]isoindole- 1,3(2H,5H)-dione |
| E 32 | 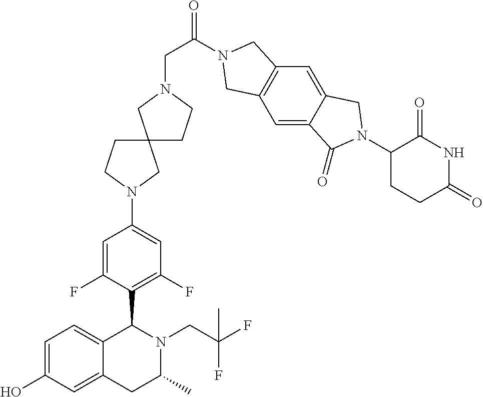 [SMILES: C[C@@H]1Cc2cc(O)ccc2[C@@H](c2c(F)cc(N3CCC4(CCN(CC(=O)N5Cc6cc7c(cc6C5)C(=O)N(C5CCC(=O)NC5=O)C7)C4)C3)cc2F)N1CC(C)(F)F] | 3-(6-(2-(7-(4-((1S,3R)-2-(2,2- difluoropropyl)-6-hydroxy-3-methyl- 1,2,3,4-tetrahydroisoquinolin-1-yl)-3,5- difluorophenyl)-2,7- diazaspiro[4.4]nonan-2-yl)acetyl)-1- oxo-3,5,6,7-tetrahydropyrrolo[3,4- f]isoindol-2(1H)-yl)piperidine-2,6-dione |
| E 33 | 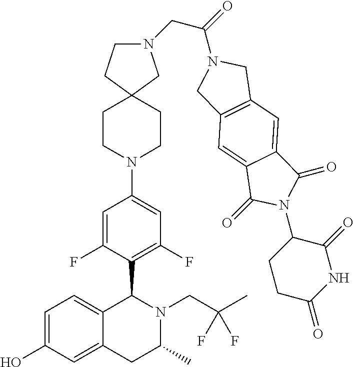 [SMILES: C[C@@H]1Cc2cc(O)ccc2[C@@H](c2c(F)cc(N3CCC4(CCN(CC(=O)N5Cc6cc7c(cc6C5)C(=O)N(C5CCC(=O)NC5=O)C7=O)C4)CC3)cc2F)N1CC(C)(F)F] | 6-(2-(8-(4-((1S,3R)-2-(2,2- difluoropropyl)-6-hydroxy-3-methyl- 1,2,3,4-tetrahydroisoquinolin-1-yl)-3,5- difluorophenyl)-2,8- diazaspiro[4.5]decan-2-yl)acetyl)-2- (2,6-dioxopiperidin-3-yl)-6,7- dihydropyrrolo[3,4-f]isoindole- 1,3(2H,5H)-dione |
| E 34 | 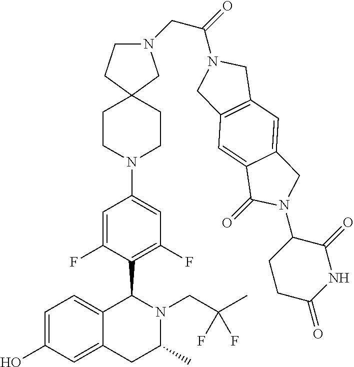 [SMILES: C[C@@H]1Cc2cc(O)ccc2[C@@H](c2c(F)cc(N3CCC4(CCN(CC(=O)N5Cc6cc7c(cc6C5)C(=O)N(C5CCC(=O)NC5=O)C7)C4)CC3)cc2F)N1CC(C)(F)F] | 3-(6-(2-(8-(4-((1S,3R)-2-(2,2- difluoropropyl)-6-hydroxy-3-methyl- 1,2,3,4-tetrahydroisoquinolin-1-yl)-3,5- difluorophenyl)-2,8- diazaspiro[4.5]decan-2-yl)acetyl)-1-oxo- 3,5,6,7-tetrahydropyrrolo[3,4-f]isoindol- 2(1H)-yl)piperidine-2,6-dione |
| E 35 | 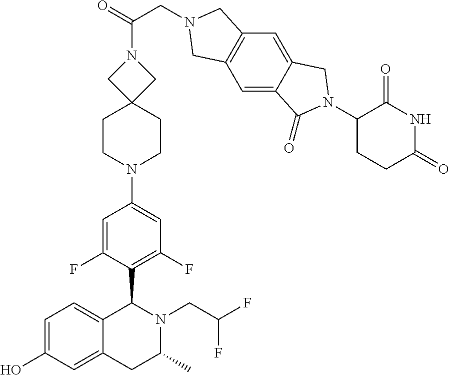 [SMILES: C[C@@H]1Cc2cc(O)ccc2[C@@H](c2c(F)cc(N3CCC4(CC3)CN(C(=O)CN3Cc5cc6c(cc5C3)C(=O)N(C3CCC(=O)NC3=O)C6)C4)cc2F)N1CC(F)F] | 3-(6-(2-(7-(4-((1S,3R)-2-(2,2- difluoroethyl)-6-hydroxy-3-methyl- 1,2,3,4-tetrahydroisoquinolin-1-yl)-3,5- difluorophenyl)-2,7- diazaspiro[3.5]nonan-2-yl)-2-oxoethyl)- 1-oxo-3,5,6,7-tetrahydropyrrolo[3,4- flisoindol-2(1H)-yl)piperidine-2,6-dione |
| E 36 | 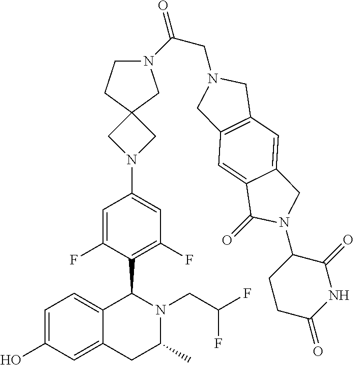 [SMILES: C[C@@H]1Cc2cc(O)ccc2[C@@H](c2c(F)cc(N3CC4(CCN(C(=O)CN5Cc6cc7c(cc6C5)C(=O)N(C5CCC(=O)NC5=O)C7)C4)C3)cc2F)N1CC(F)F] | 3-(6-(2-(2-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.4]octan-6-yl)-2-oxoethyl)- 1-oxo-3,5,6,7-tetrahydropyrrolo[3,4- f]isoindol-2(1H)-yl)piperidine-2,6-dione |
| E 37 | 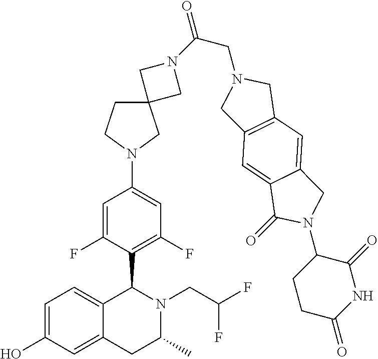 [SMILES: C[C@@H]1Cc2cc(O)ccc2[C@@H](c2c(F)cc(N3CCC4(CN(C(=O)CN5Cc6cc7c(cc6C5)C(=O)N(C5CCC(=O)NC5=O)C7)C4)C3)cc2F)N1CC(F)F] | 3-(6-(2-(6-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.4]octan-2-yl)-2-oxoethyl)- 1-oxo-3,5,6,7-tetrahydropyrrolo[3,4- f]isoindol-2(1H)-yl)piperidine-2,6-dione |
| E 38 | 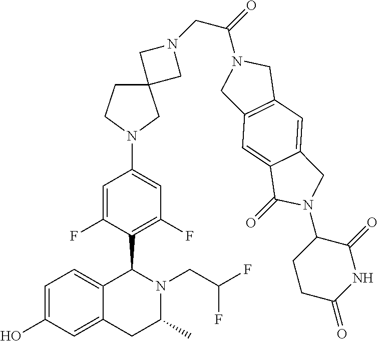 [SMILES: C[C@@H]1Cc2cc(O)ccc2[C@@H](c2c(F)cc(N3CCC4(CN(CC(=O)N5Cc6cc7c(cc6C5)C(=O)N(C5CCC(=O)NC5=O)C7)C4)C3)cc2F)N1CC(F)F] | 3-(6-(2-(6-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.4]octan-2-yl)acetyl)-1-oxo- 3,5,6,7-tetrahydropyrrolo[3,4-f]isoindol- 2(1H)-yl)piperidine-2,6-dione |
| E 39 | 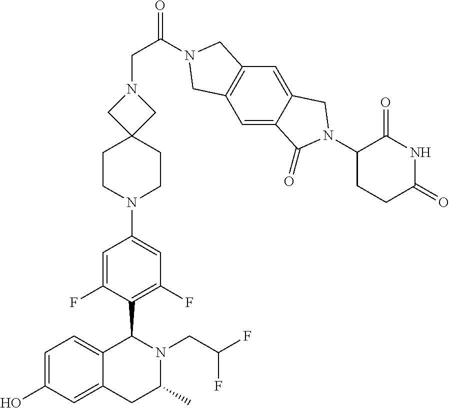 [SMILES: C[C@@H]1Cc2cc(O)ccc2[C@@H](c2c(F)cc(N3CCC4(CC3)CN(CC(=O)N3Cc5cc6c(cc5C3)C(=O)N(C3CCC(=O)NC3=O)C6)C4)cc2F)N1CC(F)F] | 3-(6-(2-(7-(4-((1S,3R)-2-(2,2- difluoroethyl)-6-hydroxy-3-methyl- 1,2,3,4-tetrahydroisoquinolin-1-yl)-3,5- difluorophenyl)-2,7- diazaspiro[3.5]nonan-2-yl)acetyl)-1- oxo-3,5,6,7-tetrahydropyrrolo[3,4- flisoindol-2(1H)-yl)piperidine-2,6-dione |
| E 40 | 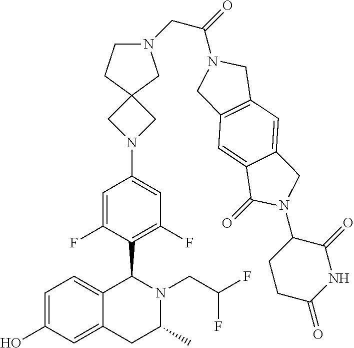 [SMILES: C[C@@H]1Cc2cc(O)ccc2[C@@H](c2c(F)cc(N3CC4(CCN(CC(=O)N5Cc6cc7c(cc6C5)C(=O)N(C5CCC(=O)NC5=O)C7)C4)C3)cc2F)N1CC(F)F] | 3-(6-(2-(2-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.4]octan-6-yl)acetyl)-1-oxo- 3,5,6,7-tetrahydropyrrolo[3,4-f]isoindol- 2(1H)-yl)piperidine-2,6-dione |
| E 41 | 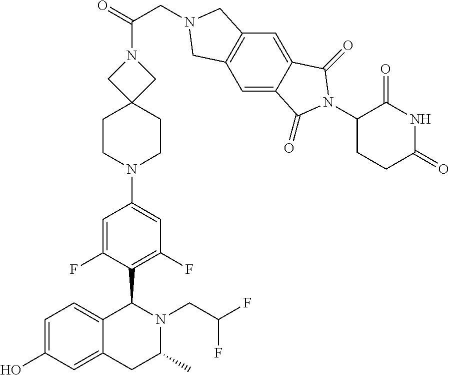 [SMILES: C[C@@H]1Cc2cc(O)ccc2[C@@H](c2c(F)cc(N3CCC4(CC3)CN(C(=O)CN3Cc5cc6c(cc5C3)C(=O)N(C3CCC(=O)NC3=O)C6=O)C4)cc2F)N1CC(F)F] | 6-(2-(7-(4-((1S,3R)-2-(2,2- difluoroethyl)-6-hydroxy-3-methyl- 1,2,3,4-tetrahydroisoquinolin-1-yl)-3,5- difluorophenyl)-2,7- diazaspiro[3.5]nonan-2-yl)-2-oxoethyl)- 2-(2,6-dioxopiperidin-3-yl)-6,7- dihydropyrrolo[3,4-f]isoindole- 1,3(2H,5H)-dione |
| E 42 | 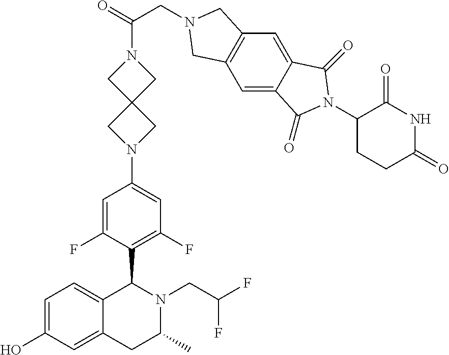 [SMILES: C[C@@H]1Cc2cc(O)ccc2[C@@H](c2c(F)cc(N3CC4(CN(C(=O)CN5Cc6cc7c(cc6C5)C(=O)N(C5CCC(=O)NC5=O)C7=O)C4)C3)cc2F)N1CC(F)F] | 6-(2-(6-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.3]heptan-2-yl)-2-oxoethyl)- 2-(2,6-dioxopiperidin-3-yl)-6,7- dihydropyrrolo[3,4-f]isoindole- 1,3(2H,5H)-dione |
| E 43 | 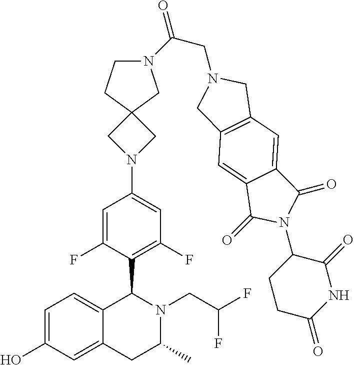 [SMILES: C[C@@H]1Cc2cc(O)ccc2[C@@H](c2c(F)cc(N3CC4(CCN(C(=O)CN5Cc6cc7c(cc6C5)C(=O)N(C5CCC(=O)NC5=O)C7=O)C4)C3)cc2F)N1CC(F)F] | 6-(2-(2-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.4]octan-6-yl)-2-oxoethyl)- 2-(2,6-dioxopiperidin-3-yl)-6,7- dihydropyrrolo[3,4-f]isoindole- 1,3(2H,5H)-dione |
| E 44 | 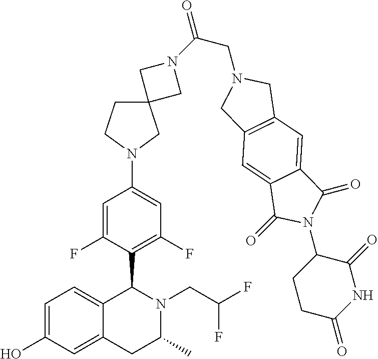 [SMILES: C[C@@H]1Cc2cc(O)ccc2[C@@H](c2c(F)cc(N3CCC4(CN(C(=O)CN5Cc6cc7c(cc6C5)C(=O)N(C5CCC(=O)NC5=O)C7=O)C4)C3)cc2F)N1CC(F)F] | 6-(2-(6-(4-((1S,3R)-2-(2,2- difluoroethyl)-6-hydroxy-3-methyl- 1,2,3,4-tetrahydroisoquinolin-1-yl)-3,5- difluorophenyl)-2,6- diazaspiro[3.4]octan-2-yl)-2-oxoethyl)- 2-(2,6-dioxopiperidin-3-yl)-6,7- dihydropyrrolo[3,4-f]isoindole- 1,3(2H,5H)-dione |
| E 45 | 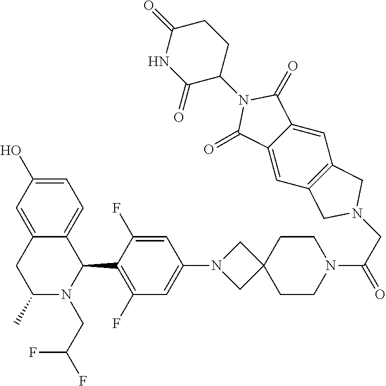 [SMILES: C[C@@H]1Cc2cc(O)ccc2[C@@H](c2c(F)cc(N3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1S,3R)-2-(2,2- difluoroethyl)-6-hydroxy-3-methyl- 1,2,3,4-tetrahydroisoquinolin-1-yl)-3,5- difluorophenyl)-2,7- diazaspiro[3.5]nonan-7-yl)-2-oxoethyl)- 2-(2,6-dioxopiperidin-3-yl)-6,7- dihydropyrrolo[3,4-f]isoindole- 1,3(2H,5H)-dione |
| E 46 | 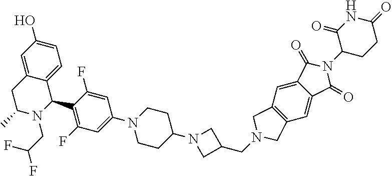 [SMILES: C[C@@H]1Cc2cc(O)ccc2[C@@H](c2c(F)cc(N3CCC(N4CC(CN5Cc6cc7c(cc6C5)C(=O)N(C5CCC(=O)NC5=O)C7=O)C4)CC3)cc2F)N1CC(F)F] | 6-((1-(1-(4-((1S,3R)-2-(2,2- difluoroethyl)-6-hydroxy-3-methyl- 1,2,3,4-tetrahydroisoquinolin-1-yl)-3,5- difluorophenyl)piperidin-4-yl)azetidin- 3-yl)methyl)-2-(2,6-dioxopiperidin-3- yl)-6,7-dihydropyrrolo[3,4-f]isoindole- 1,3(2H,5H)-dione |
| E 47 | 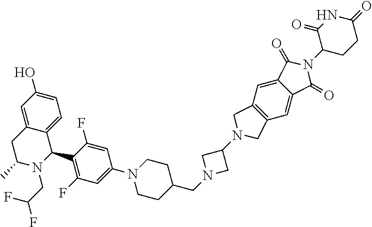 [SMILES: C[C@@H]1Cc2cc(O)ccc2[C@@H](c2c(F)cc(N3CCC(CN4CC(N5Cc6cc7c(cc6C5)C(=O)N(C5CCC(=O)NC5=O)C7=O)C4)CC3)cc2F)N1CC(F)F] | 6-(1-((1-(4-((1S,3R)-2-(2,2- difluoroethyl)-6-hydroxy-3-methyl- 1,2,3,4-tetrahydroisoquinolin-1-yl)-3,5- difluorophenyl)piperidin-4- yl)methyl)azetidin-3-yl)-2-(2,6- dioxopiperidin-3-yl)-6,7- dihydropyrrolo[3,4-f]isoindole- 1,3(2H,5H)-dione |
| E 48 | 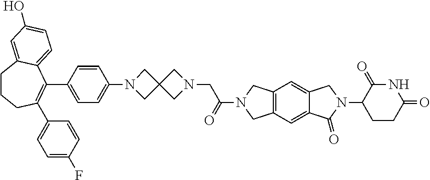 [SMILES: O=C1CCC(N2Cc3cc4c(cc3C2=O)CN(C(=O)CN2CC3(C2)CN(c2ccc(C5=C(c6ccc(F)cc6)CCCc6cc(O)ccc65)cc2)C3)C4)C(=O)N1] | 3-(6-(2-(6-(4-(8-(4-fluorophenyl)-3- hydroxy-6,7-dihydro-5H- benzo[7]annulen-9-yl)phenyl)-2,6- diazaspiro[3.3]heptan-2-yl)acetyl)-1- oxo-3,5,6,7-tetrahydropyrrolo[3,4- flisoindol-2(1H)-yl)piperidine-2,6-dione |
| E 49 | 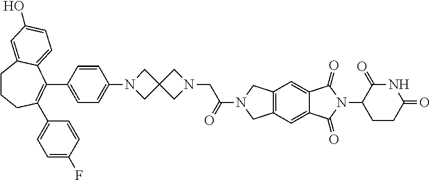 [SMILES: O=C1CCC(N2C(=O)c3cc4c(cc3C2=O)CN(C(=O)CN2CC3(C2)CN(c2ccc(C5=C(c6ccc(F)cc6)CCCc6cc(O)ccc65)cc2)C3)C4)C(=O)N1] | 2-(2,6-dioxopiperidin-3-yl)-6-(2-(6-(4- (8-(4-fluorophenyl)-3-hydroxy-6,7- dihydro-5H-benzo[7]annulen-9- yl)phenyl)-2,6-diazaspiro[3.3]heptan-2- yl)acetyl)-6,7-dihydropyrrolo[3,4- f]isoindole-1,3(2H,5H)-dione |
| E 50 | 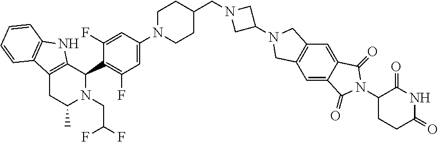 [SMILES: C[C@@H]1Cc2c([nH]c3ccccc23)[C@@H](c2c(F)cc(N3CCC(CN4CC(N5Cc6cc7c(cc6C5)C(=O)N(C5CCC(=O)NC5=O)C7=O)C4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)azetidin-3-yl)-2-(2,6- dioxopiperidin-3-yl)-6,7- dihydropyrrolo[3,4-f]isoindole- 1,3(2H,5H)-dione |
| E 51 | 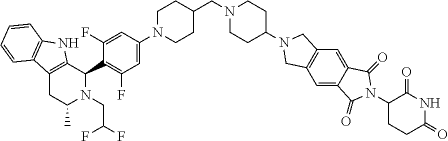 [SMILES: C[C@@H]1Cc2c([nH]c3ccccc23)[C@@H](c2c(F)cc(N3CCC(CN4CCC(N5Cc6cc7c(cc6C5)C(=O)N(C5CCC(=O)NC5=O)C7=O)CC4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 52 | 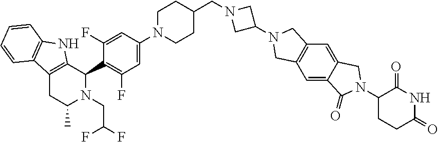 [SMILES: C[C@@H]1Cc2c([nH]c3ccccc23)[C@@H](c2c(F)cc(N3CCC(CN4CC(N5Cc6cc7c(cc6C5)C(=O)N(C5CCC(=O)NC5=O)C7)C4)CC3)cc2F)N1CC(F)F] | 3-(6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)azetidin-3-yl)-1-oxo-3,5,6,7- tetrahydropyrrolo[3,4-f]isoindol-2(1H)- yl)piperidine-2,6-dione |
| E 53 | 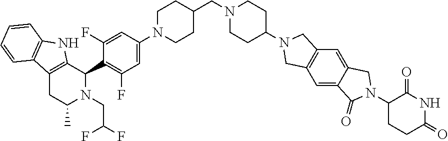 [SMILES: C[C@@H]1Cc2c([nH]c3ccccc23)[C@@H](c2c(F)cc(N3CCC(CN4CCC(N5Cc6cc7c(cc6C5)C(=O)N(C5CCC(=O)NC5=O)C7)CC4)CC3)cc2F)N1CC(F)F] | 3-(6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)piperidin-4-yl)-1-oxo-3,5,6,7- tetrahydropyrrolo[3,4-f]isoindol-2(1H)- yl)piperidine-2,6-dione |
| E 54 | 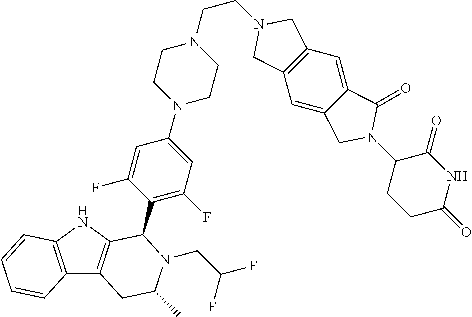 [SMILES: C[C@@H]1Cc2c([nH]c3ccccc23)[C@@H](c2c(F)cc(N3CCN(CCN4Cc5cc6c(cc5C4)C(=O)N(C4CCC(=O)NC4=O)C6)CC3)cc2F)N1CC(F)F] | 3-(6-(2-(4-(4-((1R,3R)-2-(2,2- difluoroethyl)-3-methyl-2,3,4,9- tetrahydro-1H-pyrido[3,4-b]indol-1-yl)- 3,5-difluorophenyl)piperazin-1- yl)ethyl)-1-oxo-3,5,6,7- tetrahydropyrrolo[3,4-f]isoindol-2(1H)- yl)piperidine-2,6-dione |
| E 55 | 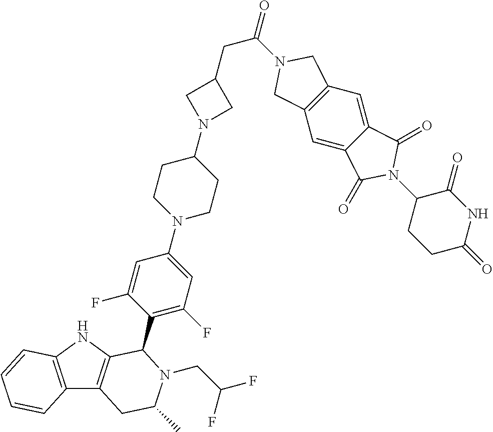 [SMILES: C[C@@H]1Cc2c([nH]c3ccccc23)[C@@H](c2c(F)cc(N3CCC(N4CC(CC(=O)N5Cc6cc7c(cc6C5)C(=O)N(C5CCC(=O)NC5=O)C7=O)C4)CC3)cc2F)N1CC(F)F] | 6-(2-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)azetidin-3-yl)acetyl)-2-(2,6- dioxopiperidin-3-yl)-6,7- dihydropyrrolo[3,4-f]isoindole- 1,3(2H,5H)-dione |
| E 56 | 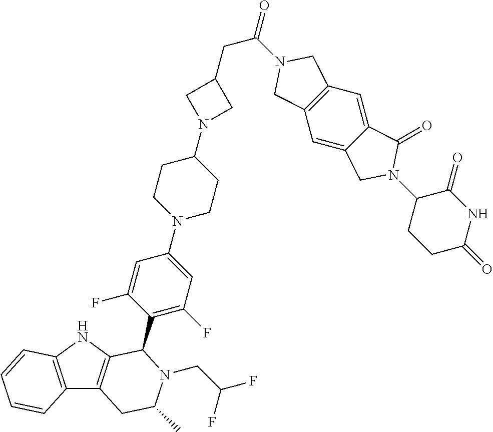 [SMILES: C[C@@H]1Cc2c([nH]c3ccccc23)[C@@H](c2c(F)cc(N3CCC(N4CC(CC(=O)N5Cc6cc7c(cc6C5)C(=O)N(C5CCC(=O)NC5=O)C7)C4)CC3)cc2F)N1CC(F)F] | 3-(6-(2-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)azetidin-3-yl)acetyl)-1-oxo-3,5,6,7- tetrahydropyrrolo[3,4-f]isoindol-2(1H)- yl)piperidine-2,6-dione |
| E 57 | 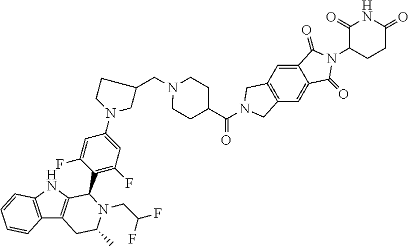 [SMILES: C[C@@H]1Cc2c([nH]c3ccccc23)[C@@H](c2c(F)cc(N3CCC(CN4CCC(C(=O)N5Cc6cc7c(cc6C5)C(=O)N(C5CCC(=O)NC5=O)C7=O)CC4)C3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)pyrrolidin-3- yl)methyl)piperidine-4-carbonyl)-2-(2,6- dioxopiperidin-3-yl)-6,7- dihydropyrrolo[3,4-f]isoindole- 1,3(2H,5H)-dione |
| E 58 | 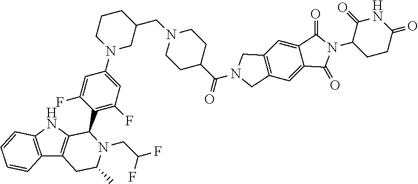 [SMILES: C[C@@H]1Cc2c([nH]c3ccccc23)[C@@H](c2c(F)cc(N3CCCC(CN4CCC(C(=O)N5Cc6cc7c(cc6C5)C(=O)N(C5CCC(=O)NC5=O)C7=O)CC4)C3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-3- yl)methyl)piperidine-4-carbonyl)-2-(2,6- dioxopiperidin-3-yl)-6,7- dihydropyrrolo[3,4-f]isoindole- 1,3(2H,5H)-dione |
| E 59 | 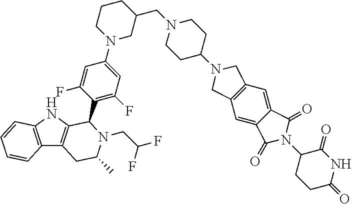 [SMILES: C[C@@H]1Cc2c([nH]c3ccccc23)[C@@H](c2c(F)cc(N3CCCC(CN4CCC(N5Cc6cc7c(cc6C5)C(=O)N(C5CCC(=O)NC5=O)C7=O)CC4)C3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-3- yl)methyl)piperidin-4-yl)-2-(2,6- dioxopiperidin-3-yl)-6,7- dihydropyrrolo[3,4-f]isoindole- 1,3(2H,5H)-dione |
| E 60 | 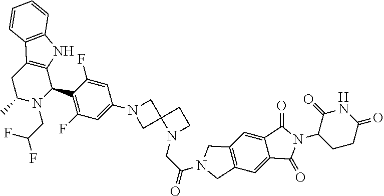 [SMILES: C[C@@H]1Cc2c([nH]c3ccccc23)[C@@H](c2c(F)cc(N3CC4(CCN4CC(=O)N4Cc5cc6c(cc5C4)C(=O)N(C4CCC(=O)NC4=O)C6=O)C3)cc2F)N1CC(F)F] | 6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-1,6- diazaspiro[3.3]heptan-1-yl)acetyl)-2- (2,6-dioxopiperidin-3-yl)-6,7- dihydropyrrolo[3,4-f]isoindole- 1,3(2H,5H)-dione |
| E 61 | 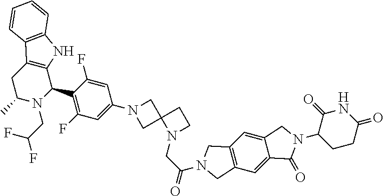 [SMILES: C[C@@H]1Cc2c([nH]c3ccccc23)[C@@H](c2c(F)cc(N3CC4(CCN4CC(=O)N4Cc5cc6c(cc5C4)C(=O)N(C4CCC(=O)NC4=O)C6)C3)cc2F)N1CC(F)F] | 3-(6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-1,6- diazaspiro[3.3]heptan-1-yl)acetyl)-1- oxo-3,5,6,7-tetrahydropyrrolo[3,4- f]isoindol-2(1H)-yl)piperidine-2,6-dione |
| E 62 | 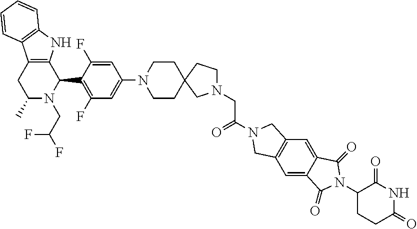 [SMILES: C[C@@H]1Cc2c([nH]c3ccccc23)[C@@H](c2c(F)cc(N3CCC4(CCN(CC(=O)N5Cc6cc7c(cc6C5)C(=O)N(C5CCC(=O)NC5=O)C7=O)C4)CC3)cc2F)N1CC(F)F] | 6-(2-(8-(4-((1R,3R)-2-(2,2- difluoroethyl)-3-methyl-2,3,4,9- tetrahydro-1H-pyrido[3,4-b]indol-1-yl)- 3,5-difluorophenyl)-2,8- diazaspiro[4.5]decan-2-yl)acetyl)-2- (2,6-dioxopiperidin-3-yl)-6,7- dihydropyrrolo[3,4-f]isoindole- 1,3(2H,5H)-dione |
| E 63 | 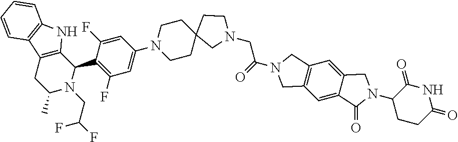 [SMILES: C[C@@H]1Cc2c([nH]c3ccccc23)[C@@H](c2c(F)cc(N3CCC4(CCN(CC(=O)N5Cc6cc7c(cc6C5)C(=O)N(C5CCC(=O)NC5=O)C7)C4)CC3)cc2F)N1CC(F)F] | 3-(6-(2-(8-(4-((1R,3R)-2-(2,2- difluoroethyl)-3-methyl-2,3,4,9- tetrahydro-1H-pyrido[3,4-b]indol-1-yl)- 3,5-difluorophenyl)-2,8- diazaspiro[4.5]decan-2-yl)acetyl)-1-oxo- 3,5,6,7-tetrahydropyrrolo[3,4-f]isoindol- 2(1H)-yl)piperidine-2,6-dione |
| E 64 | 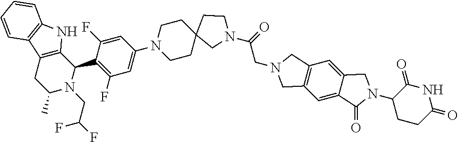 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7)C4)CC3)cc2F)N1CC(F)F] | 3-(6-(2-(8-(4-((1R,3R)-2-(2,2- difluoroethyl)-3-methyl-2,3,4,9- tetrahydro-1H-pyrido[3,4-b]indol-1-yl)- 3,5-difluorophenyl)-2,8- diazaspiro[4.5]decan-2-yl)-2-oxoethyl)- 1-oxo-3,5,6,7-tetrahydropyrrolo[3,4- f]isoindol-2(1H)-yl)piperidine-2,6-dione |
| E 65 | 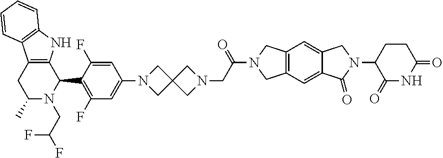 [SMILES: C[C@@H]1Cc2c([nH]c3ccccc23)[C@@H](c2c(F)cc(N3CC4(CN(CC(=O)N5Cc6cc7c(cc6C5)C(=O)N(C5CCC(=O)NC5=O)C7)C4)C3)cc2F)N1CC(F)F] | 3-(6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.3]heptan-2-yl)acetyl)-1- oxo-3,5,6,7-tetrahydropyrrolo[3,4- f]isoindol-2(1H)-yl)piperidine-2,6-dione |
| E 66 | 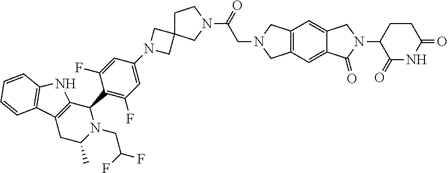 [SMILES: C[C@@H]1Cc2c([nH]c3ccccc23)[C@@H](c2c(F)cc(N3CC4(CCN(C(=O)CN5Cc6cc7c(cc6C5)C(=O)N(C5CCC(=O)NC5=O)C7)C4)C3)cc2F)N1CC(F)F] | 3-(6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.4]octan-6-yl)-2-oxoethyl)- 1-oxo-3,5,6,7-tetrahydropyrrolo[3,4- f]isoindol-2(1H)-yl)piperidine-2,6-dione |
| E 67 | 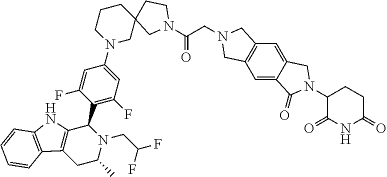 [SMILES: C[C@@H]1Cc2c([nH]c3ccccc23)[C@@H](c2c(F)cc(N3CCCC4(CCN(C(=O)CN5Cc6cc7c(cc6C5)C(=O)N(C5CCC(=O)NC5=O)C7)C4)C3)cc2F)N1CC(F)F] | 3-(6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[4.5]decan-2-yl)-2-oxoethyl)- 1-oxo-3,5,6,7-tetrahydropyrrolo[3,4- f]isoindol-2(1H)-yl)piperidine-2,6-dione |
| E 68 | 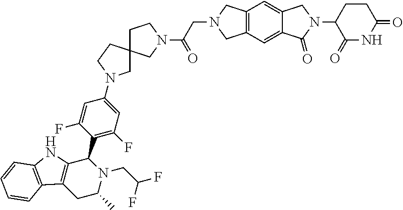 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7)C4)C3)cc2F)N1CC(F)F] | 3-(6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[4.4]nonan-2-yl)-2-oxoethyl)- 1-oxo-3,5,6,7-tetrahydropyrrolo[3,4- f]isoindol-2(1H)-yl)piperidine-2,6-dione |
| E 69 | 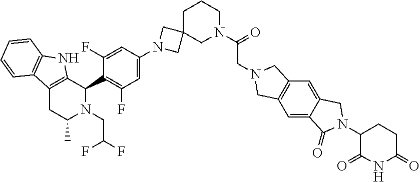 [SMILES: C[C@@H]1Cc2c([nH]c3ccccc23)[C@@H](c2c(F)cc(N3CC4(CCCN(C(=O)CN5Cc6cc7c(cc6C5)C(=O)N(C5CCC(=O)NC5=O)C7)C4)C3)cc2F)N1CC(F)F] | 3-(6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.5]nonan-6-yl)-2-oxoethyl)- 1-oxo-3,5,6,7-tetrahydropyrrolo[3,4- flisoindol-2(1H)-yl)piperidine-2,6-dione |
| E 70 | 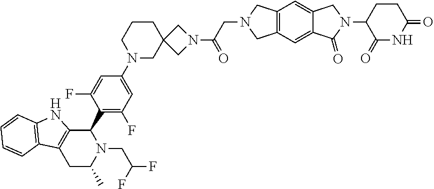 | 3-(6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.5]nonan-2-yl)-2-oxoethyl)- 1-oxo-3,5,6,7-tetrahydropyrrolo[3,4- f]isoindol-2(1H)-yl)piperidine-2,6-dione |
| E 71 | 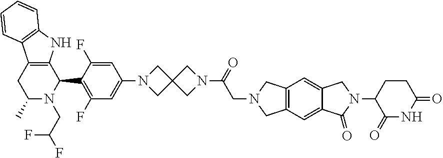 [SMILES: C[C@@H]1Cc2c([nH]c3ccccc23)[C@@H](c2c(F)cc(N3CC4(CN(C(=O)CN5Cc6cc7c(cc6C5)C(=O)N(C5CCC(=O)NC5=O)C7)C4)C3)cc2F)N1CC(F)F] | 3-(6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.3]heptan-2-yl)-2-oxoethyl)- 1-oxo-3,5,6,7-tetrahydropyrrolo[3,4- f]isoindol-2(1H)-yl)piperidine-2,6-dione |
| E 72 | 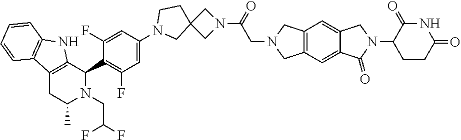 [SMILES: C[C@@H]1Cc2c([nH]c3ccccc23)[C@@H](c2c(F)cc(N3CCC4(CN(C(=O)CN5Cc6cc7c(cc6C5)C(=O)N(C5CCC(=O)NC5=O)C7)C4)C3)cc2F)N1CC(F)F] | 3-(6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.4]octan-2-yl)-2-oxoethyl)- 1-oxo-3,5,6,7-tetrahydropyrrolo[3,4- f]isoindol-2(1H)-yl)piperidine-2,6-dione |
| E 73 | 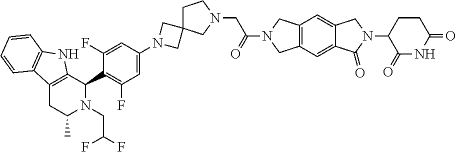 [SMILES: C[C@@H]1Cc2c([nH]c3ccccc23)[C@@H](c2c(F)cc(N3CC4(CCN(CC(=O)N5Cc6cc7c(cc6C5)C(=O)N(C5CCC(=O)NC5=O)C7)C4)C3)cc2F)N1CC(F)F] | 3-(6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.4]octan-6-yl)acetyl)-1-oxo- 3,5,6,7-tetrahydropyrrolo[3,4-f]isoindol- 2(1H)-yl)piperidine-2,6-dione |
| E 74 | 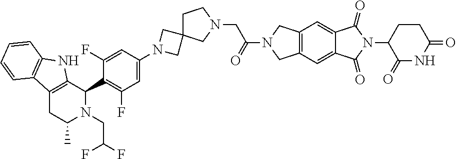 [SMILES: C[C@@H]1Cc2c([nH]c3ccccc23)[C@@H](c2c(F)cc(N3CC4(CCN(CC(=O)N5Cc6cc7c(cc6C5)C(=O)N(C5CCC(=O)NC5=O)C7=O)C4)C3)cc2F)N1CC(F)F] | 6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.4]octan-6-yl)acetyl)-2-(2,6- dioxopiperidin-3-yl)-6,7- dihydropyrrolo[3,4-f]isoindole- 1,3(2H,5H)-dione |
| E 75 | 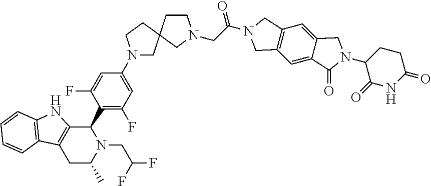 [SMILES: C[C@@H]1Cc2c([nH]c3ccccc23)[C@@H](c2c(F)cc(N3CCC4(CCN(CC(=O)N5Cc6cc7c(cc6C5)C(=O)N(C5CCC(=O)NC5=O)C7)C4)C3)cc2F)N1CC(F)F] | 3-(6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[4.4]nonan-2-yl)acetyl)-1- oxo-3,5,6,7-tetrahydropyrrolo[3,4- f]isoindol-2(1H)-yl)piperidine-2,6-dione |
| E 76 | 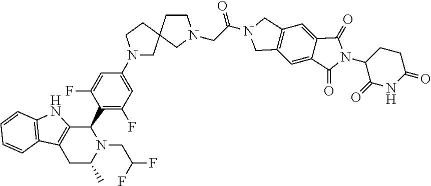 [SMILES: C[C@@H]1Cc2c([nH]c3ccccc23)[C@@H](c2c(F)cc(N3CCC4(CCN(CC(=O)N5Cc6cc7c(cc6C5)C(=O)N(C5CCC(=O)NC5=O)C7=O)C4)C3)cc2F)N1CC(F)F] | 6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[4.4]nonan-2-yl)acetyl)-2- (2,6-dioxopiperidin-3-yl)-6,7- dihydropyrrolo[3,4-f]isoindole- 1,3(2H,5H)-dione |
| E 77 | 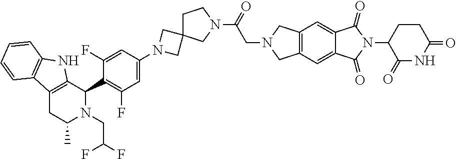 [SMILES: C[C@@H]1Cc2c([nH]c3ccccc23)[C@@H](c2c(F)cc(N3CC4(CCN(C(=O)CN5Cc6cc7c(cc6C5)C(=O)N(C5CCC(=O)NC5=O)C7=O)C4)C3)cc2F)N1CC(F)F] | 6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.4]octan-6-yl)-2-oxoethyl)- 2-(2,6-dioxopiperidin-3-yl)-6,7- dihydropyrrolo[3,4-f]isoindole- 1,3(2H,5H)-dione |
| E 78 | 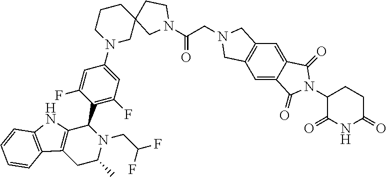 [SMILES: C[C@@H]1Cc2c([nH]c3ccccc23)[C@@H](c2c(F)cc(N3CCCC4(CCN(C(=O)CN5Cc6cc7c(cc6C5)C(=O)N(C5CCC(=O)NC5=O)C7=O)C4)C3)cc2F)N1CC(F)F] | 6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[4.5]decan-2-yl)-2-oxoethyl)- 2-(2,6-dioxopiperidin-3-yl)-6,7- dihydropyrrolo[3,4-f]isoindole- 1,3(2H,5H)-dione |
| E 79 | 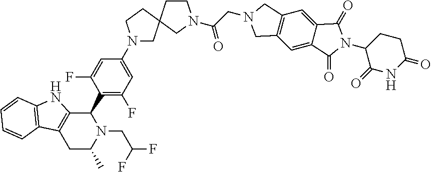 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7=O)C4)C3)cc2F)N1CC(F)F] | 6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[4.4]nonan-2-yl)-2-oxoethyl)- 2-(2,6-dioxopiperidin-3-yl)-6,7- dihydropyrrolo[3,4-f]isoindole- 1,3(2H,5H)-dione |
| E 80 | 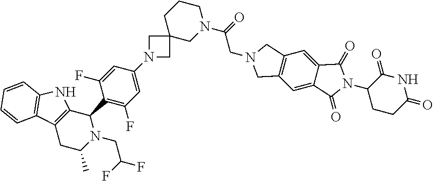 [SMILES: C[C@@H]1Cc2c([nH]c3ccccc23)[C@@H](c2c(F)cc(N3CC4(CCCN(C(=O)CN5Cc6cc7c(cc6C5)C(=O)N(C5CCC(=O)NC5=O)C7=O)C4)C3)cc2F)N1CC(F)F] | 6-(2-(2-(4-((1S,3S)-2-(2,2- difluoroethyl)-3-methyl-2,3,4,9- tetrahydro-1H-pyrido[3,4-b]indol-1-yl)- 3,5-difluorophenyl)-2,6- diazaspiro[3.5]nonan-6-yl)-2-oxoethyl)- 2-(2,6-dioxopiperidin-3-yl)-6,7- dihydropyrrolo[3,4-f]isoindole- 1,3(2H,5H)-dione |
| E 81 | 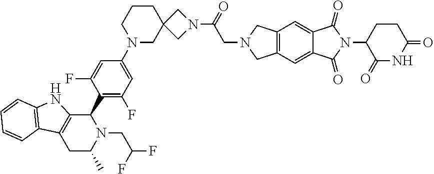 [SMILES: C[C@@H]1Cc2c([nH]c3ccccc23)[C@@H](c2c(F)cc(N3CCCC4(CN(C(=O)CN5Cc6cc7c(cc6C5)C(=O)N(C5CCC(=O)NC5=O)C7=O)C4)C3)cc2F)N1CC(F)F] | 6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.5]nonan-2-yl)-2-oxoethyl)- 2-(2,6-dioxopiperidin-3-yl)-6,7- dihydropyrrolo[3,4-f]isoindole- 1,3(2H,5H)-dione |
| E 82 | 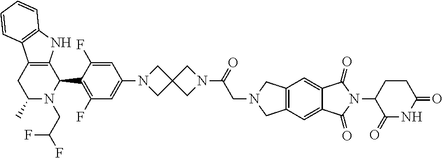 [SMILES: C[C@@H]1Cc2c([nH]c3ccccc23)[C@@H](c2c(F)cc(N3CC4(CN(C(=O)CN5Cc6cc7c(cc6C5)C(=O)N(C5CCC(=O)NC5=O)C7=O)C4)C3)cc2F)N1CC(F)F] | 6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.3]heptan-2-yl)-2-oxoethyl)- 2-(2,6-dioxopiperidin-3-yl)-6,7- dihydropyrrolo[3,4-f]isoindole- 1,3(2H,5H)-dione |
| E 83 | 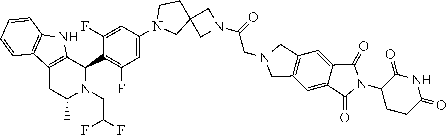 [SMILES: C[C@@H]1Cc2c([nH]c3ccccc23)[C@@H](c2c(F)cc(N3CCC4(CN(C(=O)CN5Cc6cc7c(cc6C5)C(=O)N(C5CCC(=O)NC5=O)C7=O)C4)C3)cc2F)N1CC(F)F] | 6-(2-(6-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.4]octan-2-yl)-2-oxoethyl)- 2-(2,6-dioxopiperidin-3-yl)-6,7- dihydropyrrolo[3,4-f]isoindole- 1,3(2H,5H)-dione |
| E 84 | 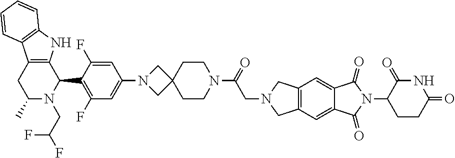 [SMILES: C[C@@H]1Cc2c([nH]c3ccccc23)[C@@H](c2c(F)cc(N3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1S,3S)-2-(2,2- difluoroethyl)-3-methyl-2,3,4,9- tetrahydro-1H-pyrido[3,4-b]indol-1-yl)- 3,5-difluorophenyl)-2,7- diazaspiro[3.5]nonan-7-yl)-2-oxoethyl)- 2-(2,6-dioxopiperidin-3-yl)-6,7- dihydropyrrolo[3,4-f]isoindole- 1,3(2H,5H)-dione |
| E 85 | 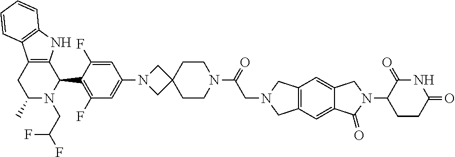 [SMILES: C[C@@H]1Cc2c([nH]c3ccccc23)[C@@H](c2c(F)cc(N3CC4(CCN(C(=O)CN5Cc6cc7c(cc6C5)C(=O)N(C5CCC(=O)NC5=O)C7)CC4)C3)cc2F)N1CC(F)F] | 3-(6-(2-(2-(4-((1S,3S)-2-(2,2- difluoroethyl)-3-methyl-2,3,4,9- tetrahydro-1H-pyrido[3,4-b]indol-1-yl)- 3,5-difluorophenyl)-2,7- diazaspiro[3.5]nonan-7-yl)-2-oxoethyl)- 1-oxo-3,5,6,7-tetrahydropyrrolo[3,4- f]isoindol-2(1H)-yl)piperidine-2,6-dione |
| E 86 | 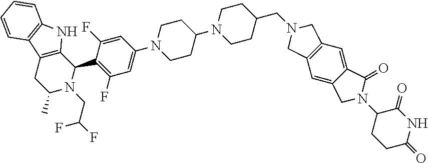 [SMILES: C[C@@H]1Cc2c([nH]c3ccccc23)[C@@H](c2c(F)cc(N3CCC(N4CCC(CN5Cc6cc7c(cc6C5)C(=O)N(C5CCC(=O)NC5=O)C7)CC4)CC3)cc2F)N1CC(F)F] | 3-(6-((1′-(4-((1R,3R)-2-(2,2- difluoroethyl)-3-methyl-2,3,4,9- tetrahydro-1H-pyrido[3,4-b]indol-1-yl)- 3,5-difluorophenyl)-[1,4′-bipiperidin]-4- yl)methyl)-1-oxo-3,5,6,7- tetrahydropyrrolo[3,4-f]isoindol-2(1H)- yl)piperidine-2,6-dione |
| E 87 | 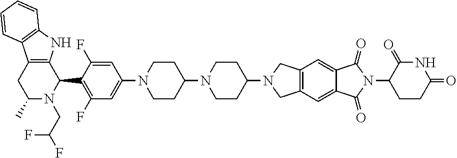 [SMILES: C[C@@H]1Cc2c([nH]c3ccccc23)[C@@H](c2c(F)cc(N3CCC(N4CCC(N5Cc6cc7c(cc6C5)C(=O)N(C5CCC(=O)NC5=O)C7=O)CC4)CC3)cc2F)N1CC(F)F] | 6-(1′-(4-((1R,3R)-2-(2,2-difluoroethyl)- 3-methyl-2,3,4,9-tetrahydro-1H- pyrido[3-4-b]indol-1-yl)-3,5- difluorophenyl)-[1,4′-bipiperidin]-4-yl)- 2-(2,6-dioxopiperidin-3-yl)-6,7- dihydropyrrolo[3,4-f]isoindole- 1,3(2H,5H)-dione |
| E 88 | 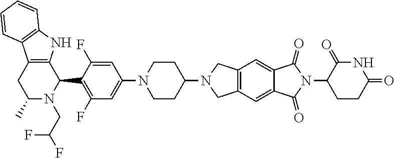 [SMILES: C[C@@H]1Cc2c([nH]c3ccccc23)[C@@H](c2c(F)cc(N3CCC(N4Cc5cc6c(cc5C4)C(=O)N(C4CCC(=O)NC4=O)C6=O)CC3)cc2F)N1CC(F)F] | 6-(1-(4-((1R,3R)-2-(2,2-difluoroethyl)- 3-methyl-2,3,4,9-tetrahydro-1H- pyrido[3,4-b]indol-1-yl)-3,5- difluorophenyl)piperidin-4-yl)-2-(2,6- dioxopiperidin-3-yl)-6,7- dihydropyrrolo[3,4-f]isoindole- 1,3(2H,5H)-dione |
| E 89 | 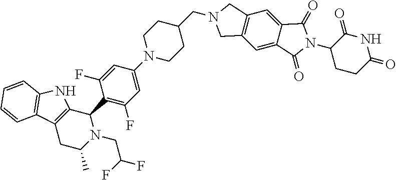 [SMILES: C[C@@H]1Cc2c([nH]c3ccccc23)[C@@H](c2c(F)cc(N3CCC(CN4Cc5cc6c(cc5C4)C(=O)N(C4CCC(=O)NC4=O)C6=O)CC3)cc2F)N1CC(F)F] | 6-((1-(4-((1R,3R)-2-(2,2-difluoroethyl)- 3-methyl-2,3,4,9-tetrahydro-1H- pyrido[3,4-b]indol-1-yl)-3,5- difluorophenyl)piperidin-4-yl)methyl)- 2-(2,6-dioxopiperidin-3-yl)-6,7- dihydropyrrolo[3,4-f]isoindole- 1,3(2H,5H)-dione |
| E 90 | 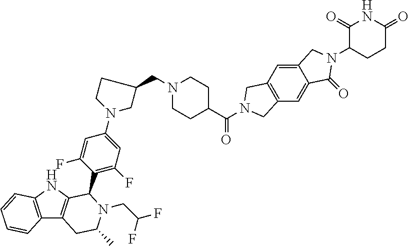 [SMILES: C[C@@H]1Cc2c([nH]c3ccccc23)[C@@H](c2c(F)cc(N3CC[C@@H](CN4CCC(C(=O)N5Cc6cc7c(cc6C5)C(=O)N(C5CCC(=O)NC5=O)C7)CC4)C3)cc2F)N1CC(F)F] | 3-(6-(1-(((S)-1-(4-((1R,3R)-2-(2,2- difluoroethyl)-3-methyl-2,3,4,9- tetrahydro-1H-pyrido[3,4-b]indol-1-yl)- 3,5-difluorophenyl)pyrrolidin-3- yl)methyl)piperidine-4-carbonyl)-1-oxo- 3,5,6,7-tetrahydropyrrolo[3,4-f]isoindol- 2(1H)-yl)piperidine-2,6-dione |
| E 91 | 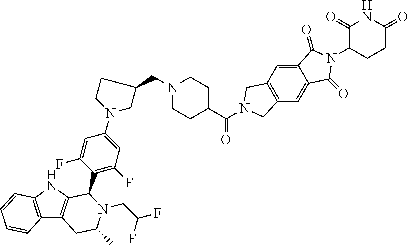 [SMILES: C[C@@H]1Cc2c([nH]c3ccccc23)[C@@H](c2c(F)cc(N3CC[C@@H](CN4CCC(C(=O)N5Cc6cc7c(cc6C5)C(=O)N(C5CCC(=O)NC5=O)C7=O)CC4)C3)cc2F)N1CC(F)F] | 6-(1-(((S)-1-(4-((1R,3R)-2-(2,2- difluoroethyl)-3-methyl-2,3,4,9- tetrahydro-1H-pyrido[3,4-b]indol-1-yl)- 3,5-difluorophenyl)pyrrolidin-3- yl)methyl)piperidine-4-carbonyl)-2-(2,6- dioxopiperidin-3-yl)-6,7- dihydropyrrolo[3,4-f]isoindole- 1,3(2H,5H)-dione |
| E 92 | 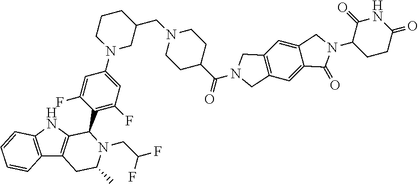 [SMILES: C[C@@H]1Cc2c([nH]c3ccccc23)[C@@H](c2c(F)cc(N3CCCC(CN4CCC(C(=O)N5Cc6cc7c(cc6C5)C(=O)N(C5CCC(=O)NC5=O)C7)CC4)C3)cc2F)N1CC(F)F] | 3-(6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-3- yl)methyl)piperidine-4-carbonyl)-1-oxo- 3,5,6,7-tetrahydropyrrolo[3,4-f]isoindol- 2(1H)-yl)piperidine-2,6-dione |
| E 93 | 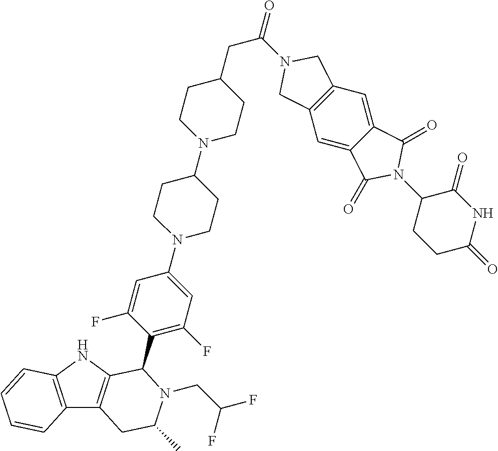 [SMILES: C[C@@H]1Cc2c([nH]c3ccccc23)[C@@H](c2c(F)cc(N3CCC(N4CCC(CC(=O)N5Cc6cc7c(cc6C5)C(=O)N(C5CCC(=O)NC5=O)C7=O)CC4)CC3)cc2F)N1CC(F)F] | 6-(2-(1′-(4-((1R,3R)-2-(2,2- difluoroethyl)-3-methyl-2,3,4,9- tetrahydro-1H-pyrido[3,4-b]indol-1-yl)- 3,5-difluorophenyl)-[1,4′-bipiperidin]-4- yl)acetyl)-2-(2,6-dioxopiperidin-3-yl)- 6,7-dihydropyrrolo[3,4-f]isoindole- 1,3(2H,5H)-dione |
| E 94 | 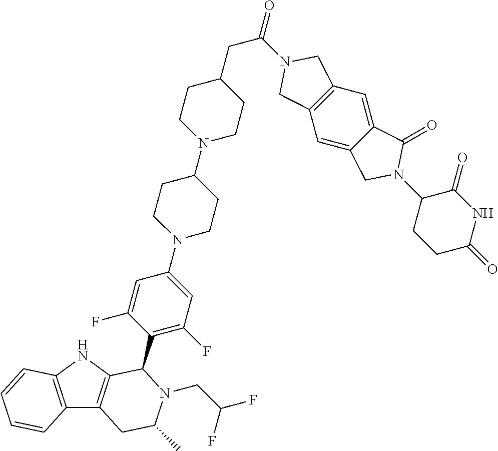 [SMILES: C[C@@H]1Cc2c([nH]c3ccccc23)[C@@H](c2c(F)cc(N3CCC(N4CCC(CC(=O)N5Cc6cc7c(cc6C5)C(=O)N(C5CCC(=O)NC5=O)C7)CC4)CC3)cc2F)N1CC(F)F] | 3-(6-(2-(1′-(4-((1R,3R)-2-(2,2- difluoroethyl)-3-methyl-2,3,4,9- tetrahydro-1H-pyrido[3,4-b]indol-1-yl)- 3,5-difluorophenyl)-[1,4′-bipiperidin]-4- yl)acetyl)-1-oxo-3,5,6,7- tetrahydropyrrolo[3,4-f]isoindol-2(1H)- yl)piperidine-2,6-dione |
| E 95 | 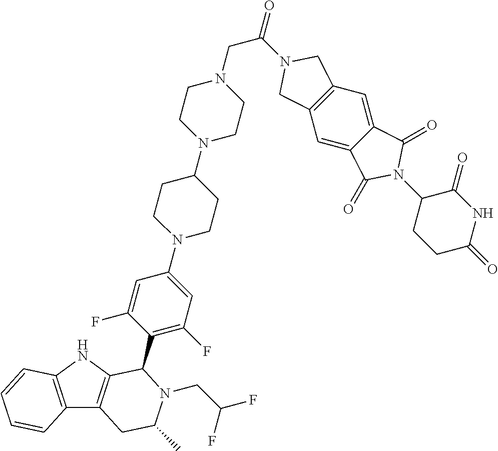 [SMILES: C[C@@H]1Cc2c([nH]c3ccccc23)[C@@H](c2c(F)cc(N3CCC(N4CCN(CC(=O)N5Cc6cc7c(cc6C5)C(=O)N(C5CCC(=O)NC5=O)C7=O)CC4)CC3)cc2F)N1CC(F)F] | 6-(2-(4-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)piperazin-1-yl)acetyl)-2-(2,6- dioxopiperidin-3-yl)-6,7- dihydropyrrolo[3,4-f]isoindole- 1,3(2H,5H)-dione |
| E 96 | 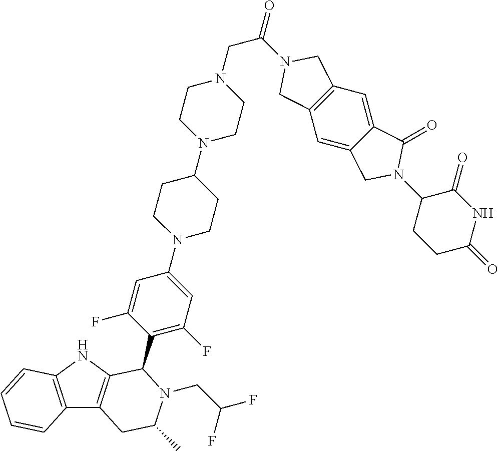 [SMILES: C[C@@H]1Cc2c([nH]c3ccccc23)[C@@H](c2c(F)cc(N3CCC(N4CCN(CC(=O)N5Cc6cc7c(cc6C5)C(=O)N(C5CCC(=O)NC5=O)C7)CC4)CC3)cc2F)N1CC(F)F] | 3-(6-(2-(4-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)piperazin-1-yl)acetyl)-1-oxo-3,5,6,7- tetrahydropyrrolo[3,4-f]isoindol-2(1H)- yl)piperidine-2,6-dione |
| E 97 | 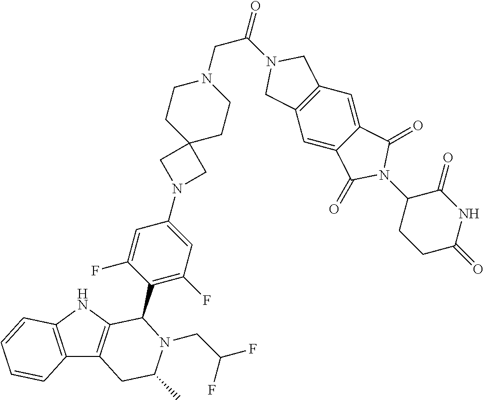 [SMILES: C[C@@H]1Cc2c([nH]c3ccccc23)[C@@H](c2c(F)cc(N3CC4(CCN(CC(=O)N5Cc6cc7c(cc6C5)C(=O)N(C5CCC(=O)NC5=O)C7=O)CC4)C3)cc2F)N1CC(F)F] | 6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[3.5]nonan-7-yl)acetyl)-2- (2,6-dioxopiperidin-3-yl)-6,7- dihydropyrrolo[3,4-f]isoindole- 1,3(2H,5H)-dione |
| E 98 | 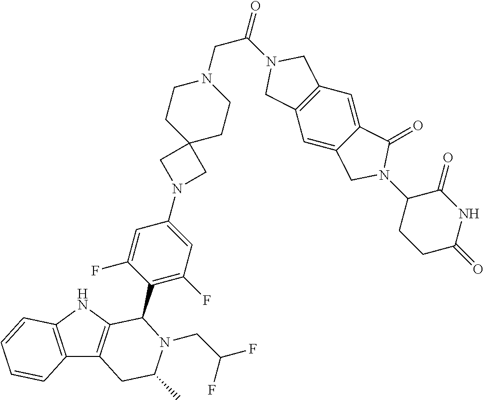 [SMILES: C[C@@H]1Cc2c([nH]c3ccccc23)[C@@H](c2c(F)cc(N3CC4(CCN(CC(=O)N5Cc6cc7c(cc6C5)C(=O)N(C5CCC(=O)NC5=O)C7)CC4)C3)cc2F)N1CC(F)F] | 3-(6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[3.5]nonan-7-yl)acetyl)-1- oxo-3,5,6,7-tetrahydropyrrolo[3,4- f]isoindol-2(1H)-yl)piperidine-2,6-dione |
| E 99 | 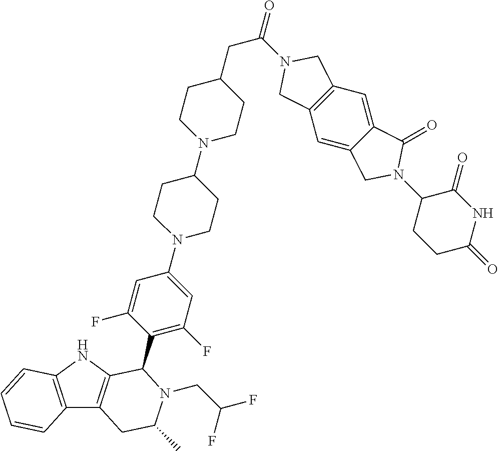 [SMILES: C[C@@H]1Cc2c([nH]c3ccccc23)[C@@H](c2c(F)cc(N3CCC(N4CCC(CC(=O)N5Cc6cc7c(cc6C5)C(=O)N(C5CCC(=O)NC5=O)C7)CC4)CC3)cc2F)N1CC(F)F] | 6-((1-(7-(4-((1R,3R)-2-(2,2- difluoroethyl)-3-methyl-2,3,4,9- tetrahydro-1H-pyrido[3,4-b]indol-1-yl)- 3,5-difluorophenyl)-7- azaspiro[3.5]nonane-2- carbonyl)piperidin-4-yl)methyl)-2-(2,6- dioxopiperidin-3-yl)-6,7- dihydropyrrolo[3,4-f]isoindole- 1,3(2H,5H)-dione |
| E 100 | 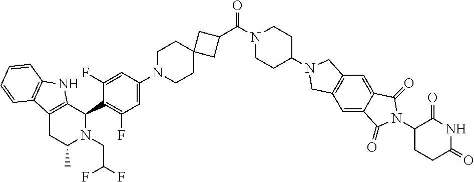 [SMILES: C[C@@H]1Cc2c([nH]c3ccccc23)[C@@H](c2c(F)cc(N3CCC4(CC3)CC(C(=O)N3CCC(N5Cc6cc7c(cc6C5)C(=O)N(C5CCC(=O)NC5=O)C7=O)CC3)C4)cc2F)N1CC(F)F] | 6-(1-(7-(4-((1R,3R)-2-(2,2- difluoroethyl)-3-methyl-2,3,4,9- tetrahydro-1H-pyrido[3,4-b]indol-1-yl)- 3,5-difluorophenyl)-7- azaspiro[3.5]nonane-2- carbonyl)piperidin-4-yl)-2-(2,6- dioxopiperidin-3-yl)-6,7- dihydropyrrolo[3,4-f]isoindole- 1,3(2H,5H)-dione |
| E 101 | 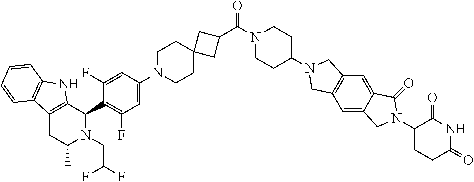 [SMILES: C[C@@H]1Cc2c([nH]c3ccccc23)[C@@H](c2c(F)cc(N3CCC4(CC3)CC(C(=O)N3CCC(N5Cc6cc7c(cc6C5)C(=O)N(C5CCC(=O)NC5=O)C7)CC3)C4)cc2F)N1CC(F)F] | 3-(6-(1-(7-(4-((1R,3R)-2-(2,2- difluoroethyl)-3-methyl-2,3,4,9- tetrahydro-1H-pyrido[3,4-b]indol-1-yl)- 3,5-difluorophenyl)-7- azaspiro[3.5]nonane-2- carbonyl)piperidin-4-yl)-1-oxo-3,5,6,7- tetrahydropyrrolo[3,4-f]isoindol-2(1H)- yl)piperidine-2,6-dione |
| E 102 | 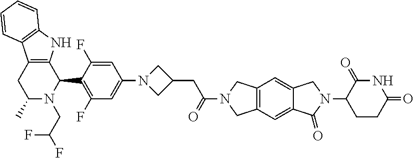 [SMILES: C[C@@H]1Cc2c([nH]c3ccccc23)[C@@H](c2c(F)cc(N3CC(CC(=O)N4Cc5cc6c(cc5C4)C(=O)N(C4CCC(=O)NC4=O)C6)C3)cc2F)N1CC(F)F] | 3-(6-(2-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidin-3-yl)acetyl)- 1-oxo-3,5,6,7-tetrahydropyrrolo[3,4- f]isoindol-2(1H)-yl)piperidine-2,6-dione |
| E 103 | 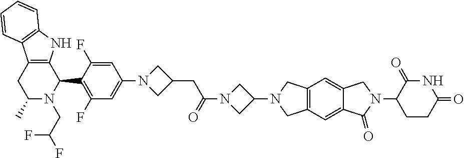 [SMILES: C[C@@H]1Cc2c([nH]c3ccccc23)[C@@H](c2c(F)cc(N3CC(CC(=O)N4CC(N5Cc6cc7c(cc6C5)C(=O)N(C5CCC(=O)NC5=O)C7)C4)C3)cc2F)N1CC(F)F] | 3-(6-(1-(2-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidin-3- yl)acetyl)azetidin-3-yl)-1-oxo-3,5,6,7- tetrahydropyrrolo[3,4-f]isoindol-2(1H)- yl)piperidine-2,6-dione |
| E 104 | 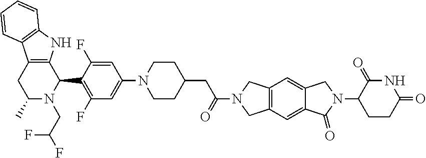 [SMILES: C[C@@H]1Cc2c([nH]c3ccccc23)[C@@H](c2c(F)cc(N3CCC(CC(=O)N4Cc5cc6c(cc5C4)C(=O)N(C4CCC(=O)NC4=O)C6)CC3)cc2F)N1CC(F)F] | 3-(6-(2-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)acetyl)-1-oxo-3,5,6,7- tetrahydropyrrolo[3,4-f]isoindol-2(1H)- yl)piperidine-2,6-dione |
| E 105 | 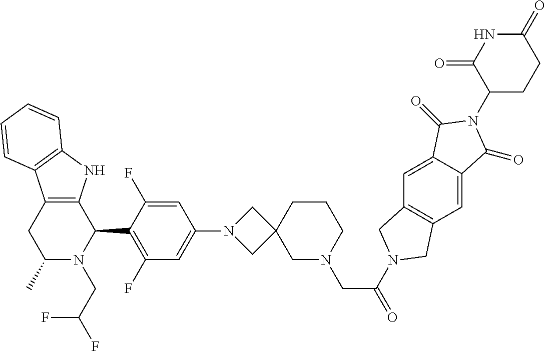 [SMILES: C[C@@H]1Cc2c([nH]c3ccccc23)[C@@H](c2c(F)cc(N3CC4(CCCN(CC(=O)N5Cc6cc7c(cc6C5)C(=O)N(C5CCC(=O)NC5=O)C7=O)C4)C3)cc2F)N1CC(F)F] | 6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.5]nonan-6-yl)acetyl)-2- (2,6-dioxopiperidin-3-yl)-6,7- dihydropyrrolo[3,4-f]isoindole- 1,3(2H,5H)-dione |
| E 106 | 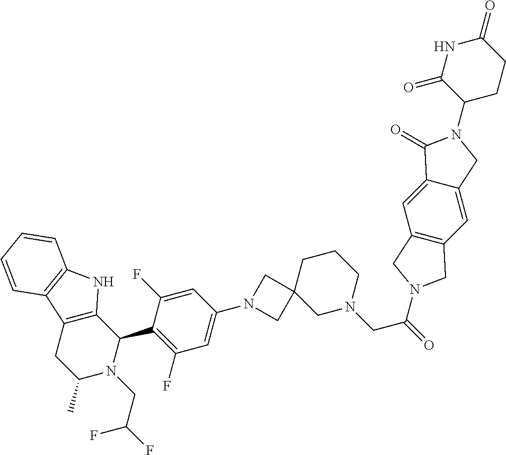 [SMILES: C[C@@H]1Cc2c([nH]c3ccccc23)[C@@H](c2c(F)cc(N3CC4(CCCN(CC(=O)N5Cc6cc7c(cc6C5)C(=O)N(C5CCC(=O)NC5=O)C7)C4)C3)cc2F)N1CC(F)F] | 3-(6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,6- diazaspiro[3.5]nonan-6-yl)acetyl)-1- oxo-3,5,6,7-tetrahydropyrrolo[3,4- f]isoindol-2(1H)-yl)piperidine-2,6-dione |
| E 107 | 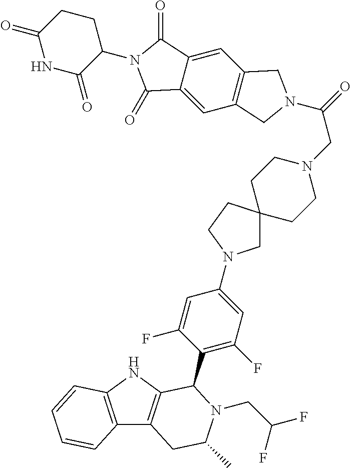 [SMILES: C[C@@H]1Cc2c([nH]c3ccccc23)[C@@H](c2c(F)cc(N3CCC4(CCN(CC(=O)N5Cc6cc7c(cc6C5)C(=O)N(C5CCC(=O)NC5=O)C7=O)CC4)C3)cc2F)N1CC(F)F] | 6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,8- diazaspiro[4.5]decan-8-yl)acetyl)-2- (2,6-dioxopiperidin-3-yl)-6,7- dihydropyrrolo[3,4-f]isoindole- 1,3(2H,5H)-dione |
| E 108 | 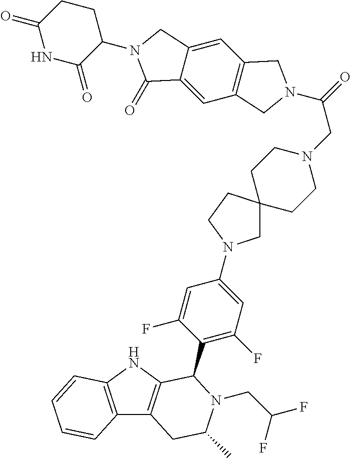 [SMILES: C[C@@H]1Cc2c([nH]c3ccccc23)[C@@H](c2c(F)cc(N3CCC4(CCN(CC(=O)N5Cc6cc7c(cc6C5)C(=O)N(C5CCC(=O)NC5=O)C7)CC4)C3)cc2F)N1CC(F)F] | 3-(6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,8- diazaspiro[4.5]decan-8-yl)acetyl)-1-oxo- 3,5,6,7-tetrahydropyrrolo[3,4-f]isoindol- 2(1H)-yl)piperidine-2,6-dione |
| E 109 | 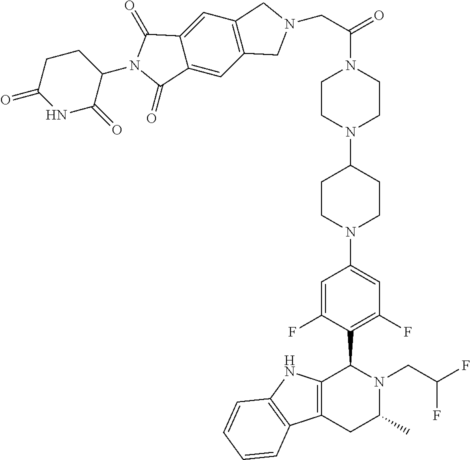 [SMILES: C[C@@H]1Cc2c([nH]c3ccccc23)[C@@H](c2c(F)cc(N3CCC(N4CCN(C(=O)CN5Cc6cc7c(cc6C5)C(=O)N(C5CCC(=O)NC5=O)C7=O)CC4)CC3)cc2F)N1CC(F)F] | 3-(6-(2-(4-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)piperazin-1-yl)-2-oxoethyl)-1-oxo- 3,5,6,7-tetrahydropyrrolo[3,4-f]isoindol- 2(1H)-yl)piperidine-2,6-dione |
| E 110 | 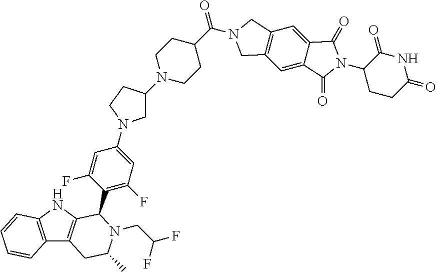 [SMILES: C[C@@H]1Cc2c([nH]c3ccccc23)[C@@H](c2c(F)cc(N3CCC(N4CCC(C(=O)N5Cc6cc7c(cc6C5)C(=O)N(C5CCC(=O)NC5=O)C7=O)CC4)C3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)pyrrolidin-3- yl)piperidine-4-carbonyl)-2-(2,6- dioxopiperidin-3-yl)-6,7- dihydropyrrolo[3,4-f]isoindole- 1,3(2H,5H)-dione |
| E 111 | 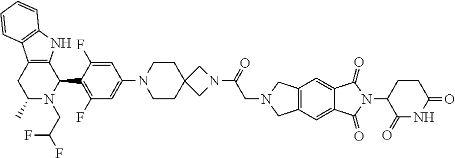 [SMILES: C[C@@H]1Cc2c([nH]c3ccccc23)[C@@H](c2c(F)cc(N3CCC4(CC3)CN(C(=O)CN3Cc5cc6c(cc5C3)C(=O)N(C3CCC(=O)NC3=O)C6=O)C4)cc2F)N1CC(F)F] | 6-(2-(7-(4-((1R,3R)-2-(2,2- difluoroethyl)-3-methyl-2,3,4,9- tetrahydro-1H-pyrido[3,4-b]indol-1-yl)- 3,5-difluorophenyl)-2,7- diazaspiro[3.5]nonan-2-yl)-2-oxoethyl)- 2-(2,6-dioxopiperidin-3-yl)-6,7- dihydropyrrolo[3,4-f]isoindole- 1,3(2H,5H)-dione |
| E 112 | 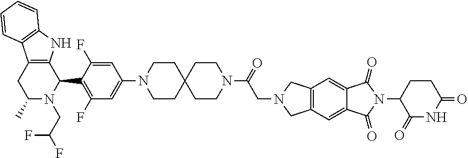 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3,5-difluorophenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 113 | 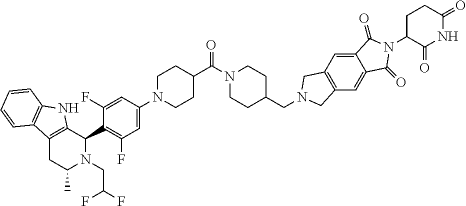 [SMILES: C[C@@H]1Cc2c([nH]c3ccccc23)[C@@H](c2c(F)cc(N3CCC(C(=O)N4CCC(CN5Cc6cc7c(cc6C5)C(=O)N(C5CCC(=O)NC5=O)C7=O)CC4)CC3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-4- carbonyl)piperidin-4-yl)methyl)-2-(2,6- dioxopiperidin-3-yl)-6,7- dihydropyrrolo[3,4-f]isoindole- 1,3(2H,5H)-dione |
| E 114 | 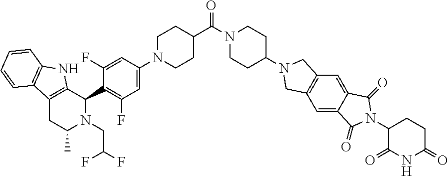 [SMILES: C[C@@H]1Cc2c([nH]c3ccccc23)[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 115 | 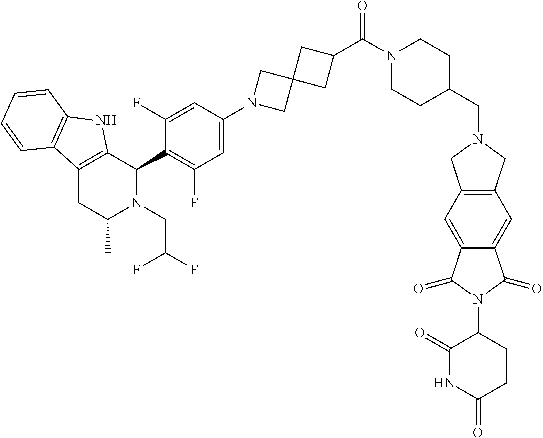 [SMILES: C[C@@H]1Cc2c([nH]c3ccccc23)[C@@H](c2c(F)cc(N3CC4(CC(C(=O)N5CCC(CN6Cc7cc8c(cc7C6)C(=O)N(C6CCC(=O)NC6=O)C8=O)CC5)C4)C3)cc2F)N1CC(F)F] | 6-((1-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2- azaspiro[3.3]heptane-6- carbonyl)piperidin-4-yl)methyl)-2-(2,6- dioxopiperidin-3-yl)-6,7- dihydropyrrolo[3,4-f]isoindole- 1,3(2H,5H)-dione |
| E 116 | 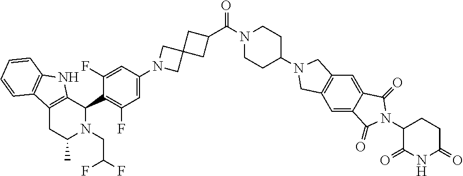 [SMILES: C[C@@H]1Cc2c([nH]c3ccccc23)[C@@H](c2c(F)cc(N3CC4(CC(C(=O)N5CCC(N6Cc7cc8c(cc7C6)C(=O)N(C6CCC(=O)NC6=O)C8=O)CC5)C4)C3)cc2F)N1CC(F)F] | 6-(1-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2- azaspiro[3.3]heptane-6- carbonyl)piperidin-4-yl)-2-(2,6- dioxopiperidin-3-yl)-6,7- dihydropyrrolo[3,4-f]isoindole- 1,3(2H,5H)-dione |
| E 117 | 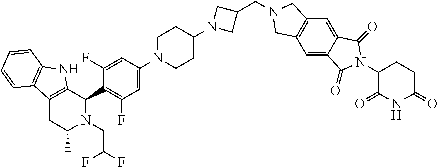 [SMILES: C[C@@H]1Cc2c([nH]c3ccccc23)[C@@H](c2c(F)cc(N3CCC(N4CC(CN5Cc6cc7c(cc6C5)C(=O)N(C5CCC(=O)NC5=O)C7=O)C4)CC3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)azetidin-3-yl)methyl)-2-(2,6- dioxopiperidin-3-yl)-6,7- dihydropyrrolo[3,4-f]isoindole- 1,3(2H,5H)-dione |
| E 118 | 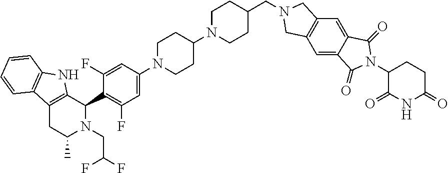 [SMILES: C[C@@H]1Cc2c([nH]c3ccccc23)[C@@H](c2c(F)cc(N3CCC(N4CCC(CN5Cc6cc7c(cc6C5)C(=O)N(C5CCC(=O)NC5=O)C7=O)CC4)CC3)cc2F)N1CC(F)F] | 6-((1′-(4-((1R,3R)-2-(2,2-difluoroethyl)- 3-methyl-2,3,4,9-tetrahydro-1H- pyrido[3,4-b]indol-1-yl)-3,5- difluorophenyl)-[1,4′-bipiperidin]-4- yl)methyl)-2-(2,6-dioxopiperidin-3-yl)- 6,7-dihydropyrrolo[3,4-f]isoindole- 1,3(2H,5H)-dione |
| E 119 | 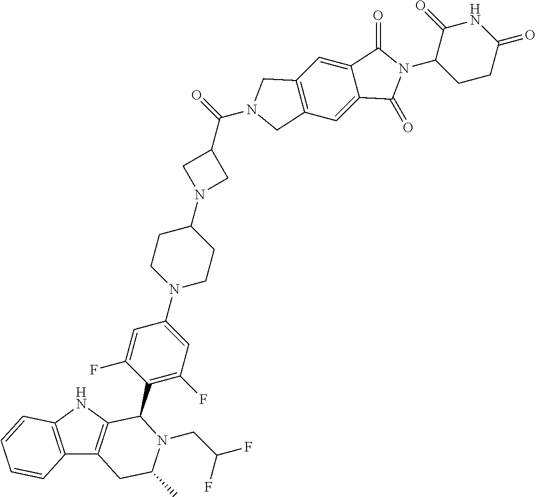 [SMILES: C[C@@H]1Cc2c([nH]c3ccccc23)[C@@H](c2c(F)cc(N3CCC(N4CC(C(=O)N5Cc6cc7c(cc6C5)C(=O)N(C5CCC(=O)NC5=O)C7=O)C4)CC3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)azetidine-3-carbonyl)-2-(2,6- dioxopiperidin-3-yl)-6,7- dihydropyrrolo[3,4-f]isoindole- 1,3(2H,5H)-dione |
| E 120 | 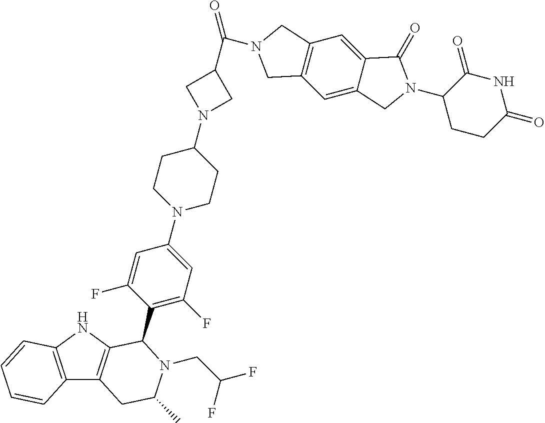 [SMILES: C[C@@H]1Cc2c([nH]c3ccccc23)[C@@H](c2c(F)cc(N3CCC(N4CC(C(=O)N5Cc6cc7c(cc6C5)C(=O)N(C5CCC(=O)NC5=O)C7)C4)CC3)cc2F)N1CC(F)F] | 3-(6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)azetidine-3-carbonyl)-1-oxo-3,5,6,7- tetrahydropyrrolo[3,4-f]isoindol-2(1H)- yl)piperidine-2,6-dione |
| E 121 | 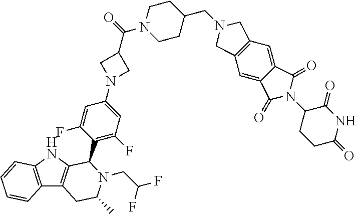 [SMILES: C[C@@H]1Cc2c([nH]c3ccccc23)[C@@H](c2c(F)cc(N3CC(C(=O)N4CCC(CN5Cc6cc7c(cc6C5)C(=O)N(C5CCC(=O)NC5=O)C7=O)CC4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)piperidin-4-yl)methyl)-2-(2,6- dioxopiperidin-3-yl)-6,7- dihydropyrrolo[3,4-f]isoindole- 1,3(2H,5H)-dione |
| E 122 | 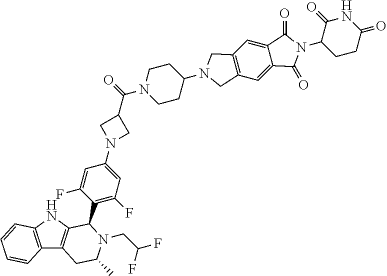 [SMILES: C[C@@H]1Cc2c([nH]c3ccccc23)[C@@H](c2c(F)cc(N3CC(C(=O)N4CCC(N5Cc6cc7c(cc6C5)C(=O)N(C5CCC(=O)NC5=O)C7=O)CC4)C3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)piperidin-4-yl)-2-(2,6- dioxopiperidin-3-yl)-6,7- dihydropyrrolo[3,4-f]isoindole- 1,3(2H,5H)-dione |
| E 123 | 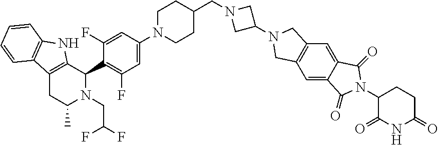 [SMILES: C[C@@H]1Cc2c([nH]c3ccccc23)[C@@H](c2c(F)cc(N3CCC(CN4CC(N5Cc6cc7c(cc6C5)C(=O)N(C5CCC(=O)NC5=O)C7=O)C4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)azetidin-3-yl)-2-(2,6- dioxopiperidin-3-yl)-6,7- dihydropyrrolo[3,4-f]isoindole- 1,3(2H,5H)-dione |
| E 124 | 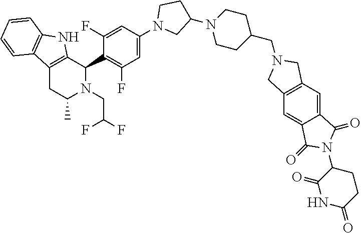 [SMILES: C[C@@H]1Cc2c([nH]c3ccccc23)[C@@H](c2c(F)cc(N3CCC(N4CCC(CN5Cc6cc7c(cc6C5)C(=O)N(C5CCC(=O)NC5=O)C7=O)CC4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)pyrrolidin-3- yl)piperidin-4-yl)methyl)-2-(2,6- dioxopiperidin-3-yl)-6,7- dihydropyrrolo[3,4-f]isoindole- 1,3(2H,5H)-dione |
| E 125 | 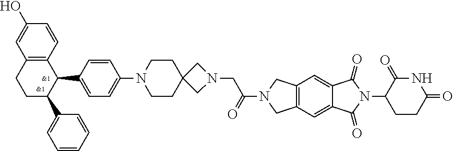 [SMILES: O=C1CCC(N2C(=O)c3cc4c(cc3C2=O)CN(C(=O)CN2CC3(CCN(c5ccc([C@@H]6c7ccc(O)cc7CC[C@@H]6c6ccccc6)cc5)CC3)C2)C4)C(=O)N1] | Rac-2-(2,6-dioxopiperidin-3-yl)-6-(2-(7- (4-((1R,2S)-6-hydroxy-2-phenyl- 1,2,3,4-tetrahydronaphthalen-1- yl)phenyl)-2,7-diazaspiro[3.5]nonan-2- yl)acetyl)-6,7-dihydropyrrolo[3,4- f]isoindole-1,3(2H,5H)-dione |
| E 126 | 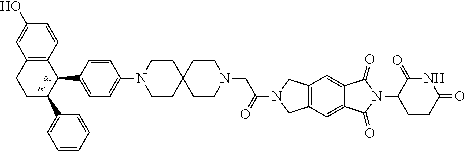 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2)CC3)C4)C(=O)N1] | Rac-2-(2,6-dioxopiperidin-3-yl)-6-(2-(9- (4-((1R,2S)-6-hydroxy-2-phenyl- 1,2,3,4-tetrahydronaphthalen-1- yl)phenyl)-3,9-diazaspiro[5.5]undecan- 3-yl)acetyl)-6,7-dihydropyrrolo[3,4- f]isoindole-1,3(2H,5H)-dione |
| E 127 | 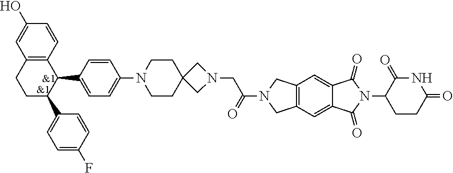 [SMILES: O=C1CCC(N2C(=O)c3cc4c(cc3C2=O)CN(C(=O)CN2CC3(CCN(c5ccc([C@@H]6c7ccc(O)cc7CC[C@@H]6c6ccc(F)cc6)cc5)CC3)C2)C4)C(=O)N1] | Rac-2-(2,6-dioxopiperidin-3-yl)-6-(2-(7- (4-((1R,2S)-2-(4-fluorophenyl)-6- hydroxy-1,2,3,4-tetrahydronaphthalen- 1-yl)phenyl)-2,7-diazaspiro[3.5]nonan- 2-yl)acetyl)-6,7-dihydropyrrolo[3,4- f]isoindole-1,3(2H,5H)-dione |
| E 128 | 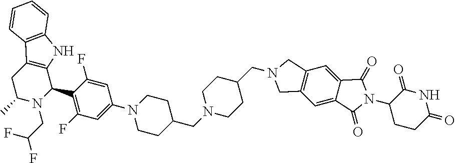 [SMILES: C[C@@H]1Cc2c([nH]c3ccccc23)[C@@H](c2c(F)cc(N3CCC(CN4CCC(CN5Cc6cc7c(cc6C5)C(=O)N(C5CCC(=O)NC5=O)C7=O)CC4)CC3)cc2F)N1CC(F)F] | 6-((1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)piperidin-4-yl)methyl)-2-(2,6- dioxopiperidin-3-yl)-6,7- dihydropyrrolo[3,4-f]isoindole- 1,3(2H,5H)-dione |
| E 129 | 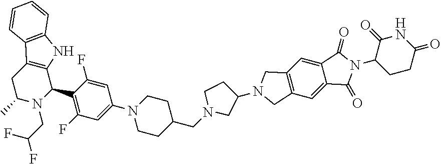 [SMILES: C[C@@H]1Cc2c([nH]c3ccccc23)[C@@H](c2c(F)cc(N3CCC(CN4CCC(N5Cc6cc7c(cc6C5)C(=O)N(C5CCC(=O)NC5=O)C7=O)C4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)pyrrolidin-3-yl)-2-(2,6- dioxopiperidin-3-yl)-6,7- dihydropyrrolo[3,4-f]isoindole- 1,3(2H,5H)-dione |
| E 130 | 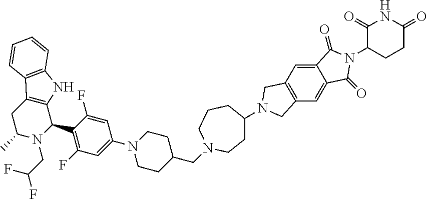 [SMILES: C[C@@H]1Cc2c([nH]c3ccccc23)[C@@H](c2c(F)cc(N3CCC(CN4CCCC(N5Cc6cc7c(cc6C5)C(=O)N(C5CCC(=O)NC5=O)C7=O)CC4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)azepan-4-yl)-2-(2,6- dioxopiperidin-3-yl)-6,7- dihydropyrrolo[3,4-f]isoindole- 1,3(2H,5H)-dione |
| E 131 | 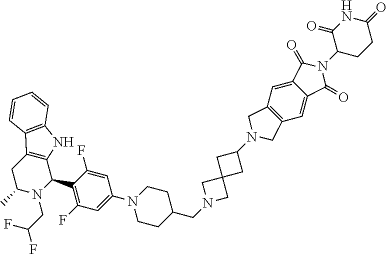 [SMILES: C[C@@H]1Cc2c([nH]c3ccccc23)[C@@H](c2c(F)cc(N3CCC(CN4CC5(CC(N6Cc7cc8c(cc7C6)C(=O)N(C6CCC(=O)NC6=O)C8=O)C5)C4)CC3)cc2F)N1CC(F)F] | 6-(2-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)-2-azaspiro[3.3]heptan-6-yl)- 2-(2,6-dioxopiperidin-3-yl)-6,7- dihydropyrrolo[3,4-f]isoindole- 1,3(2H,5H)-dione |
| E 132 | 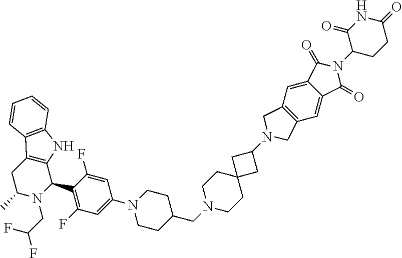 [SMILES: C[C@@H]1Cc2c([nH]c3ccccc23)[C@@H](c2c(F)cc(N3CCC(CN4CCC5(CC4)CC(N4Cc6cc7c(cc6C4)C(=O)N(C4CCC(=O)NC4=O)C7=O)C5)CC3)cc2F)N1CC(F)F] | 6-(7-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)-7-azaspiro[3.5]nonan-2-yl)- 2-(2,6-dioxopiperidin-3-yl)-6,7- dihydropyrrolo[3,4-f]isoindole- 1,3(2H,5H)-dione |
| E 133 | 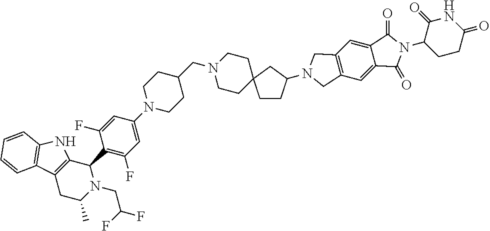 [SMILES: C[C@@H]1Cc2c([nH]c3ccccc23)[C@@H](c2c(F)cc(N3CCC(CN4CCC5(CCC(N6Cc7cc8c(cc7C6)C(=O)N(C6CCC(=O)NC6=O)C8=O)C5)CC4)CC3)cc2F)N1CC(F)F] | 6-(8-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)-8-azaspiro[4.5]decan-2-yl)- 2-(2,6-dioxopiperidin-3-yl)-6,7- dihydropyrrolo[3,4-f]isoindole- 1,3(2H,5H)-dione |
| E 134 | 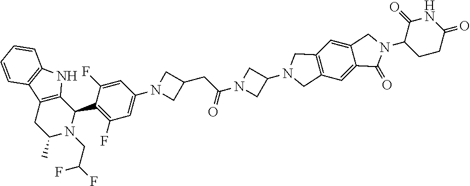 [SMILES: C[C@@H]1Cc2c([nH]c3ccccc23)[C@@H](c2c(F)cc(N3CC(CC(=O)N4CC(N5Cc6cc7c(cc6C5)C(=O)N(C5CCC(=O)NC5=O)C7)C4)C3)cc2F)N1CC(F)F] | 3-(6-(1-(2-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidin-3- yl)acetyl)azetidin-3-yl)-1-oxo-3,5,6,7- tetrahydropyrrolo[3,4-f]isoindol-2(1H)- yl)piperidine-2,6-dione |
| E 135 | 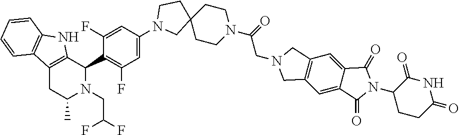 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1R,3R)-2-(2,2- difluoroethyl)-3-methyl-2,3,4,9- tetrahydro-1H-pyrido[3,4-b]indol-1-yl)- 3,5-difluorophenyl)-2,8- diazaspiro[4.5]decan-8-yl)-2-oxoethyl)- 2-(2,6-dioxopiperidin-3-yl)-6,7- dihydropyrrolo[3,4-f]isoindole- 1,3(2H,5H)-dione |
| E 136 | 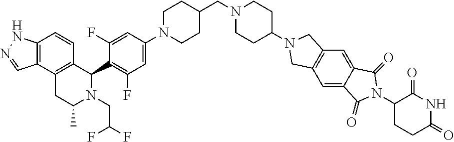 [SMILES: C[C@@H]1Cc2c(ccc3[nH]ncc23)[C@@H](c2c(F)cc(N3CCC(CN4CCC(N5Cc6cc7c(cc6C5)C(=O)N(C5CCC(=O)NC5=O)C7=O)CC4)CC3)cc2F)N1CC(F)F] | 6-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 137 | 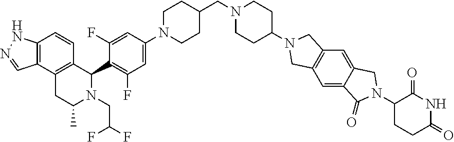 [SMILES: C[C@@H]1Cc2c(ccc3[nH]ncc23)[C@@H](c2c(F)cc(N3CCC(CN4CCC(N5Cc6cc7c(cc6C5)C(=O)N(C5CCC(=O)NC5=O)C7)CC4)CC3)cc2F)N1CC(F)F] | 3-(6-(1-((1-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperidin-4- yl)methyl)piperidin-4-yl)-1-oxo-3,5,6,7- tetrahydropyrrolo[3,4-f]isoindol-2(1H)- yl)piperidine-2,6-dione |
| E 138 | 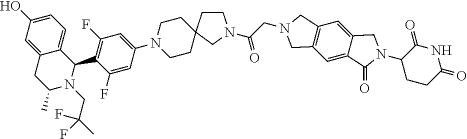 [SMILES: C[C@@H]1Cc2cc(O)ccc2[C@@H](c2c(F)cc(N3CCC4(CCN(C(=O)CN5Cc6cc7c(cc6C5)C(=O)N(C5CCC(=O)NC5=O)C7)C4)CC3)cc2F)N1CC(C)(F)F] | 3-(6-(2-(8-(4-((1S,3R)-2-(2,2- difluoropropyl)-6-hydroxy-3-methyl- 1,2,3,4-tetrahydroisoquinolin-1-yl)-3,5- difluorophenyl)-2,8- diazaspiro[4.5]decan-2-yl)-2-oxoethyl)- 1-oxo-3,5,6,7-tetrahydropyrrolo[3,4- flisoindol-2(1H)-yl)piperidine-2,6-dione |
| E 139 | 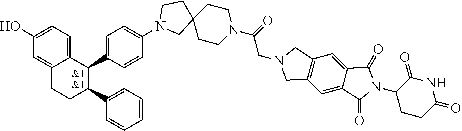 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)C3)C4)C(=O)N1] | Rac-2-(2,6-dioxopiperidin-3-yl)-6-(2-(2- (4-((1R,2S)-6-hydroxy-2-phenyl- 1,2,3,4-tetrahydronaphthalen-1- yl)phenyl)-2,8-diazaspiro[4.5]decan-8- yl)-2-oxoethyl)-6,7-dihydropyrrolo[3,4- f]isoindole-1,3(2H,5H)-dione |
| E 140 | 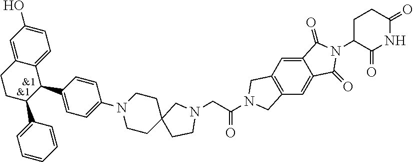 [SMILES: O=C1CCC(N2C(=O)c3cc4c(cc3C2=O)CN(C(=O)CN2CCC3(CCN(c5ccc([C@@H]6c7ccc(O)cc7CC[C@@H]6c6ccccc6)cc5)CC3)C2)C4)C(=O)N1] | Rac-2-(2,6-dioxopiperidin-3-yl)-6-(2-(8- (4-((1R,2S)-6-hydroxy-2-phenyl- 1,2,3,4-tetrahydronaphthalen-1- yl)phenyl)-2,8-diazaspiro[4.5]decan-2- yl)acetyl)-6,7-dihydropyrrolo[3,4- f]isoindole-1,3(2H,5H)-dione |
| E 141 | 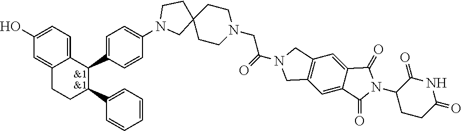 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2)C3)C4)C(=O)N1] | Rac-2-(2,6-dioxopiperidin-3-yl)-6-(2-(2- (4-((1R,2S)-6-hydroxy-2-phenyl- 1,2,3,4-tetrahydronaphthalen-1- yl)phenyl)-2,8-diazaspiro[4.5]decan-8- yl)acetyl)-6,7-dihydropyrrolo[3,4- f]isoindole-1,3(2H,5H)-dione |
| E 142 | 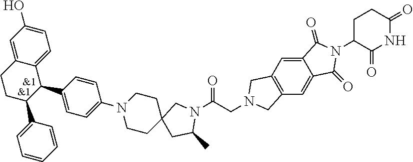 [SMILES: C[C@H]1CC2(CCN(c3ccc([C@@H]4c5ccc(O)cc5CC[C@@H]4c4ccccc4)cc3)CC2)CN1C(=O)CN1Cc2cc3c(cc2C1)C(=O)N(C1CCC(=O)NC1=O)C3=O] | Rac-2-(2,6-dioxopiperidin-3-yl)-6-(2- ((S)-8-(4-((1R,2S)-6-hydroxy-2-phenyl- 1,2,3,4-tetrahydronaphthalen-1- yl)phenyl)-3-methyl-2,8- diazaspiro[4.5]decan-2-yl)-2-oxoethyl)- 6,7-dihydropyrrolo[3,4-f]isoindole- 1,3(2H,5H)-dione |
| E 143 | 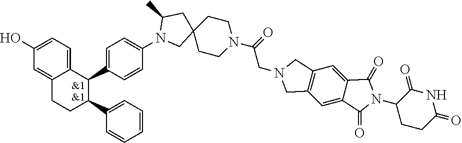 [SMILES: C[C@H]1CC2(CCN(C(=O)CN3Cc4cc5c(cc4C3)C(=O)N(C3CCC(=O)NC3=O)C5=O)CC2)CN1c1ccc([C@@H]2c3ccc(O)cc3CC[C@@H]2c2ccccc2)cc1] | Rac-2-(2,6-dioxopiperidin-3-yl)-6-(2- ((S)-2-(4-((1R,2S)-6-hydroxy-2-phenyl- 1,2,3,4-tetrahydronaphthalen-1- yl)phenyl)-3-methyl-2,8- diazaspiro[4.5]decan-8-yl)-2-oxoethyl)- 6,7-dihydropyrrolo[3,4-f]isoindole- 1,3(2H,5H)-dione |
| E 144 | 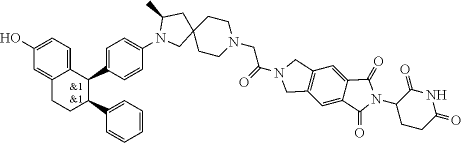 [SMILES: C[C@H]1CC2(CCN(CC(=O)N3Cc4cc5c(cc4C3)C(=O)N(C3CCC(=O)NC3=O)C5=O)CC2)CN1c1ccc([C@@H]2c3ccc(O)cc3CC[C@@H]2c2ccccc2)cc1] | Rac-2-(2,6-dioxopiperidin-3-yl)-6-(2- ((S)-2-(4-((1R,2S)-6-hydroxy-2-phenyl- 1,2,3,4-tetrahydronaphthalen-1- yl)phenyl)-3-methyl-2,8- diazaspiro[4.5]decan-8-yl)acetyl)-6,7- dihydropyrrolo[3,4-f]isoindole- 1,3(2H,5H)-dione |
| E 145 | 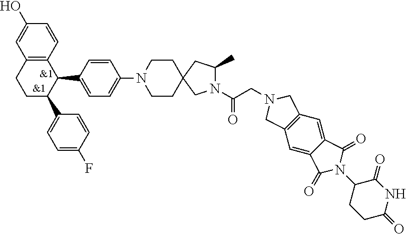 [SMILES: C[C@@H]1CC2(CCN(c3ccc([C@@H]4c5ccc(O)cc5CC[C@@H]4c4ccc(F)cc4)cc3)CC2)CN1C(=O)CN1Cc2cc3c(cc2C1)C(=O)N(C1CCC(=O)NC1=O)C3=O] | Rac-2-(2,6-dioxopiperidin-3-yl)-6-(2- ((R)-8-(4-((1R,2S)-2-(4-fluorophenyl)- 6-hydroxy-1,2,3,4- tetrahydronaphthalen-1-yl)phenyl)-3- methyl-2,8-diazaspiro[4.5]decan-2-yl)- 2-oxoethyl)-6,7-dihydropyrrolo[3,4- f]isoindole-1,3(2H,5H)-dione |
| E 146 | 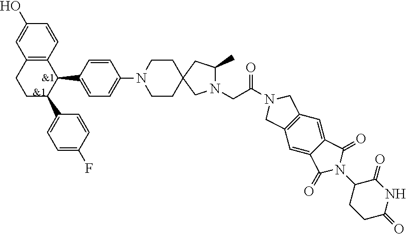 [SMILES: C[C@@H]1CC2(CCN(c3ccc([C@@H]4c5ccc(O)cc5CC[C@@H]4c4ccc(F)cc4)cc3)CC2)CN1CC(=O)N1Cc2cc3c(cc2C1)C(=O)N(C1CCC(=O)NC1=O)C3=O] | Rac-2-(2,6-dioxopiperidin-3-yl)-6-(2- ((R)-8-(4-((1R,2S)-2-(4-fluorophenyl)- 6-hydroxy-1,2,3,4- tetrahydronaphthalen-1-yl)phenyl)-3- methyl-2,8-diazaspiro[4.5]decan-2- yl)acetyl)-6,7-dihydropyrrolo[3,4- f]isoindole-1,3(2H,5H)-dione |
| E 147 | 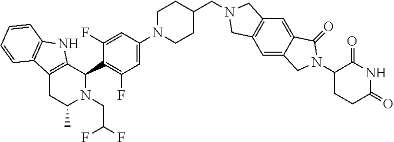 [SMILES: C[C@@H]1Cc2c([nH]c3ccccc23)[C@@H](c2c(F)cc(N3CCC(CN4Cc5cc6c(cc5C4)C(=O)N(C4CCC(=O)NC4=O)C6)CC3)cc2F)N1CC(F)F] | 3-(6-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)-1-oxo-3,5,6,7- tetrahydropyrrolo[3,4-f]isoindol-2(1H)- yl)piperidine-2,6-dione |
| E 148 | 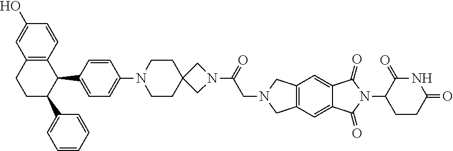 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(7-(4- ((1R,2S)-6-hydroxy-2-phenyl-1,2,3,4- tetrahydronaphthalen-1-yl)phenyl)-2,7- diazaspiro[3.5]nonan-2-yl)-2-oxoethyl)- 6,7-dihydropyrrolo[3,4-f]isoindole- 1,3(2H,5H)-dione |
| E 149 | 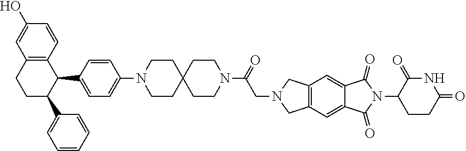 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4- ((1R,2S)-6-hydroxy-2-phenyl-1,2,3,4- tetrahydronaphthalen-1-yl)phenyl)-3,9- diazaspiro[5.5]undecan-3-yl)-2- oxoethyl)-6,7-dihydropyrrolo[3,4- f]isoindole-1,3(2H,5H)-dione |
| E 150 | 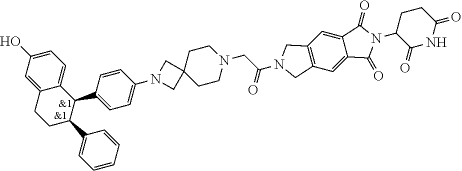 [SMILES: O=C1CCC(N2C(=O)c3cc4c(cc3C2=O)CN(C(=O)CN2CCC3(CC2)CN(c2ccc([C@@H]5c6ccc(O)cc6CC[C@@H]5c5ccccc5)cc2)C3)C4)C(=O)N1] | Rac-2-(2,6-dioxopiperidin-3-yl)-6-(2-(2- (4-((1R,2S)-6-hydroxy-2-phenyl- 1,2,3,4-tetrahydronaphthalen-1- yl)phenyl)-2,7-diazaspiro[3.5]nonan-7- yl)acetyl)-6,7-dihydropyrrolo[3,4- f]isoindole-1,3(2H,5H)-dione |
| E 151 | 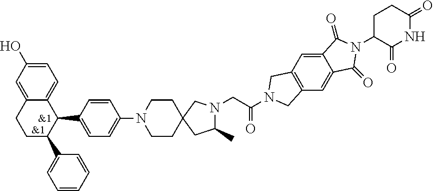 [SMILES: C[C@H]1CC2(CCN(c3ccc([C@@H]4c5ccc(O)cc5CC[C@@H]4c4ccccc4)cc3)CC2)CN1CC(=O)N1Cc2cc3c(cc2C1)C(=O)N(C1CCC(=O)NC1=O)C3=O] | Rac-2-(2,6-dioxopiperidin-3-yl)-6-(2- ((S)-8-(4-((1R,2S)-6-hydroxy-2-phenyl- 1,2,3,4-tetrahydronaphthalen-1- yl)phenyl)-3-methyl-2,8- diazaspiro[4.5]decan-2-yl)acetyl)-6,7- dihydropyrrolo[3,4-f]isoindole- 1,3(2H,5H)-dione |
| E 152 | 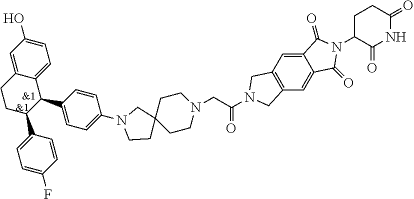 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccc(F)cc5)cc2)C3)C4)C(=O)N1] | Rac-2-(2,6-dioxopiperidin-3-yl)-6-(2-(2- (4-((1R,2S)-2-(4-fluorophenyl)-6- hydroxy-1,2,3,4-tetrahydronaphthalen- 1-yl)phenyl)-2,8-diazaspiro[4.5]decan- 8-yl)acetyl)-6,7-dihydropyrrolo[3,4- f]isoindole-1,3(2H,5H)-dione |
| E 153 | 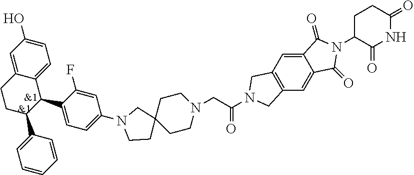 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)c(F)c2)C3)C4)C(=O)N1] | Rac-2-(2,6-dioxopiperidin-3-yl)-6-(2-(2- (3-fluoro-4-((1S,2S)-6-hydroxy-2- phenyl-1,2,3,4-tetrahydronaphthalen-1- yl)phenyl)-2,8-diazaspiro[4.5]decan-8- yl)acetyl)-6,7-dihydropyrrolo[3,4- f]isoindole-1,3(2H,5H)-dione |
| E 154 | 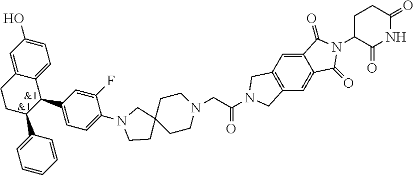 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2F)C3)C4)C(=O)N1] | Rac-2-(2,6-dioxopiperidin-3-yl)-6-(2-(2- (2-fluoro-4-((1R,2S)-6-hydroxy-2- phenyl-1,2,3,4-tetrahydronaphthalen-1- yl)phenyl)-2,8-diazaspiro[4.5]decan-8- yl)acetyl)-6,7-dihydropyrrolo[3,4- f]isoindole-1,3(2H,5H)-dione |
| E 155 | 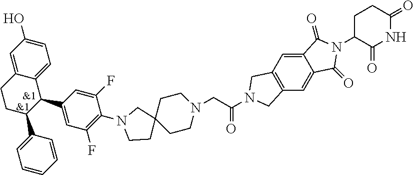 [SMILES: O=C1CCC(N2C(=O)c3cc4c(cc3C2=O)CN(C(=O)CN2CCC3(CC2)CCN(c2c(F)cc([C@@H]5c6ccc(O)cc6CC[C@@H]5c5ccccc5)cc2F)C3)C4)C(=O)N1] | Rac-6-(2-(2-(2,6-difluoro-4-((1R,2S)-6- hydroxy-2-phenyl-1,2,3,4- tetrahydronaphthalen-1-yl)phenyl)-2,8- diazaspiro[4.5]decan-8-yl)acetyl)-2- (2,6-dioxopiperidin-3-yl)-6,7- dihydropyrrolo[3,4-f]isoindole- 1,3(2H,5H)-dione |
| E 156 | 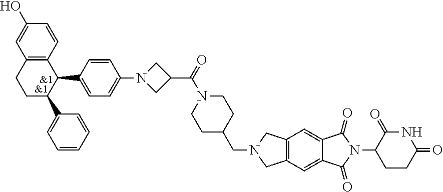 [SMILES: O=C1CCC(N2C(=O)c3cc4c(cc3C2=O)CN(CC2CCN(C(=O)C3CN(c5ccc([C@@H]6c7ccc(O)cc7CC[C@@H]6c6ccccc6)cc5)C3)CC2)C4)C(=O)N1] | Rac-2-(2,6-dioxopiperidin-3-yl)-6-((1- (1-(4-((1R,2S)-6-hydroxy-2-phenyl- 1,2,3,4-tetrahydronaphthalen-1- yl)phenyl)azetidine-3- carbonyl)piperidin-4-yl)methyl)-6,7- dihydropyrrolo[3,4-f]isoindole- 1,3(2H,5H)-dione |
| E 157 | 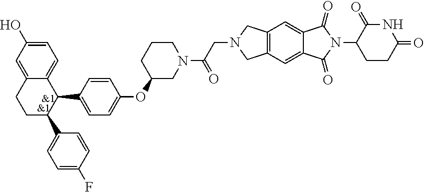 [SMILES: O=C1CCC(N2C(=O)c3cc4c(cc3C2=O)CN(CC(=O)N2CCC[C@H](Oc3ccc([C@@H]5c6ccc(O)cc6CC[C@@H]5c5ccc(F)cc5)cc3)C2)C4)C(=O)N1] | Rac-2-(2,6-dioxopiperidin-3-yl)-6-(2- ((S)-3-(4-((1R,2S)-2-(4-fluorophenyl)-6- hydroxy-1,2,3,4-tetrahydronaphthalen- 1-yl)phenoxy)piperidin-1-yl)-2- oxoethyl)-6,7-dihydropyrrolo[3,4- f]isoindole-1,3(2H,5H)-dione |
| E 158 | 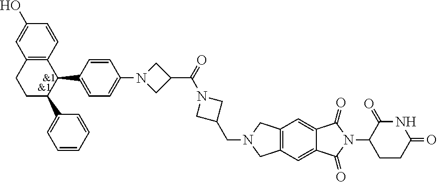 [SMILES: O=C1CCC(N2C(=O)c3cc4c(cc3C2=O)CN(CC2CN(C(=O)C3CN(c5ccc([C@@H]6c7ccc(O)cc7CC[C@@H]6c6ccccc6)cc5)C3)C2)C4)C(=O)N1] | Rac-2-(2,6-dioxopiperidin-3-yl)-6-((1- (1-(4-((1R,2S)-6-hydroxy-2-phenyl- 1,2,3,4-tetrahydronaphthalen-1- yl)phenyl)azetidine-3-carbonyl)azetidin- 3-yl)methyl)-6,7-dihydropyrrolo[3,4- f]isoindole-1,3(2H,5H)-dione |
| E 159 | 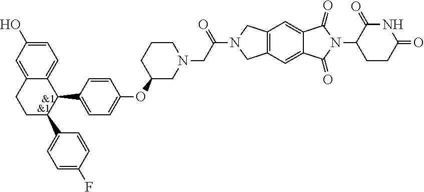 [SMILES: O=C1CCC(N2C(=O)c3cc4c(cc3C2=O)CN(C(=O)CN2CCC[C@H](Oc3ccc([C@@H]5c6ccc(O)cc6CC[C@@H]5c5ccc(F)cc5)cc3)C2)C4)C(=O)N1] | Rac-2-(2,6-dioxopiperidin-3-yl)-6-(2- ((S)-3-(4-((1R,2S)-2-(4-fluorophenyl)-6- hydroxy-1,2,3,4-tetrahydronaphthalen- 1-yl)phenoxy)piperidin-1-yl)acetyl)-6,7- dihydropyrrolo[3,4-f]isoindole- 1,3(2H,5H)-dione |
| E 160 | 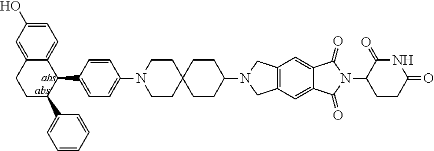 [SMILES: O=C1CCC(N2C(=O)c3cc4c(cc3C2=O)CN(C2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(3-(4- ((1R,2S)-6-hydroxy-2-phenyl-1,2,3,4- tetrahydronaphthalen-1-yl)phenyl)-3- azaspiro[5.5]undecan-9-yl)-6,7- dihydropyrrolo[3,4-f]isoindole- 1,3(2H,5H)-dione |
| E161 | 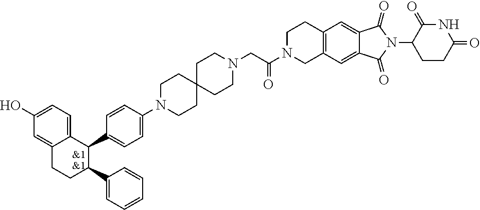 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2)CC3)CC4)C(=O)N1] | rac-2-(2,6-dioxopiperidin-3-yl)-6-(2-(9- (4-((1R,2S)-6-hydroxy-2-phenyl- 1,2,3,4-tetrahydronaphthalen-1- yl)phenyl)-3,9-diazaspiro[5.5]undecan- 3-yl)acetyl)-5,6,7,8-tetrahydro-1H- pyrrolo[3,4-g]isoquinoline-1,3(2H)- dione |
| E162 | 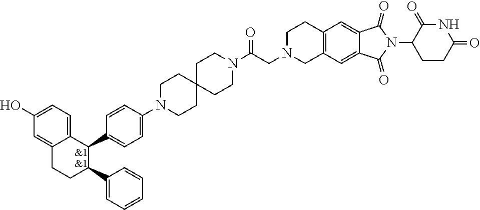 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)CC4)C(=O)N1] | rac-2-(2,6-dioxopiperidin-3-yl)-6-(2-(9- (4-((1R,2S)-6-hydroxy-2-phenyl- 1,2,3,4-tetrahydronaphthalen-1- yl)phenyl)-3,9-diazaspiro[5.5]undecan- 3-yl)-2-oxoethyl)-5,6,7,8-tetrahydro-1H- pyrrolo[3,4-g]isoquinoline-1,3(2H)- dione |
| E163 | 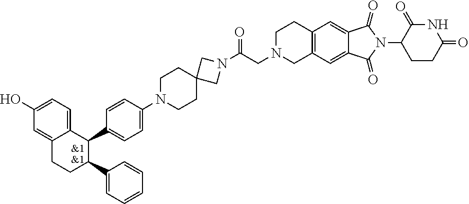 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)CC4)C(=O)N1] | rac-2-(2,6-dioxopiperidin-3-yl)-6-(2-(7- (4-((1R,2S)-6-hydroxy-2-phenyl- 1,2,3,4-tetrahydronaphthalen-1- yl)phenyl)-2,7-diazaspiro[3.5]nonan-2- yl)-2-oxoethyl)-5,6,7,8-tetrahydro-1H- pyrrolo[3,4-g]isoquinoline-1,3(2H)- dione |
| E164 | 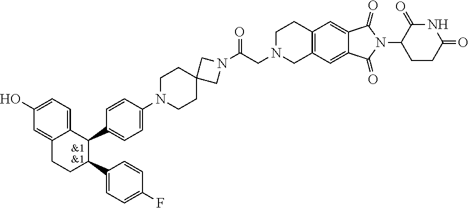 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccc(F)cc6)cc5)CC3)C2)CC4)C(=O)N1] | rac-2-(2,6-dioxopiperidin-3-yl)-6-(2-(7- (4-((1R,2S)-2-(4-fluorophenyl)-6- hydroxy-1,2,3,4-tetrahydronaphthalen- 1-yl)phenyl)-2,7-diazaspiro[3.5]nonan- 2-yl)-2-oxoethyl)-5,6,7,8-tetrahydro-1H- pyrrolo[3,4-g]isoquinoline-1,3(2H)- dione |
| E165 | 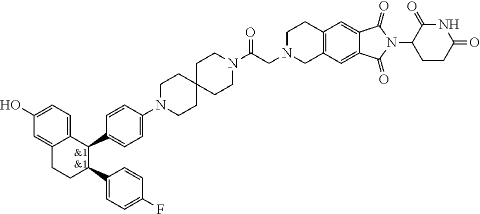 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccc(F)cc5)cc2)CC3)CC4)C(=O)N1] | rac-2-(2,6-dioxopiperidin-3-yl)-6-(2-(9- (4-((1R,2S)-2-(4-fluorophenyl)-6- hydroxy-1,2,3,4-tetrahydronaphthalen- 1-yl)phenyl)-3,9- diazaspiro[5.5]undecan-3-yl)-2- oxoethyl)-5,6,7,8-tetrahydro-1H- pyrrolo[3,4-g]isoquinoline-1,3(2H)- dione |
| E 166 | 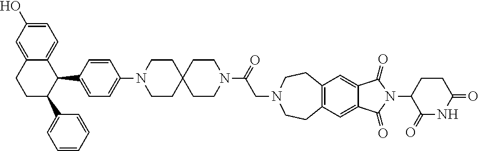 [SMILES: O=C1CCC(N2C(=O)c3cc4c(cc3C2=O)CCN(CC(=O)N2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-7-(2-(9-(4- ((1R,2S)-6-hydroxy-2-phenyl-1,2,3,4- tetrahydronaphthalen-1-yl)phenyl)-3,9- diazaspiro[5.5]undecan-3-yl)-2- oxoethyl)-6,7,8,9-tetrahydroazepino[4,5- f]isoindole-1,3(2H,5H)-dione |
| E 167 | 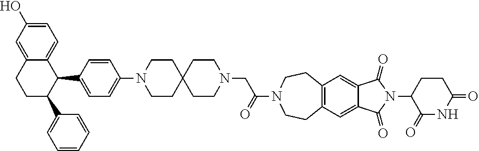 [SMILES: O=C1CCC(N2C(=O)c3cc4c(cc3C2=O)CCN(C(=O)CN2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-7-(2-(9-(4- ((1R,2S)-6-hydroxy-2-phenyl-1,2,3,4- tetrahydronaphthalen-1-yl)phenyl)-3,9- diazaspiro[5.5]undecan-3-yl)acetyl)- 6,7,8,9-tetrahydroazepino[4,5- f]isoindole-1,3(2H,5H)-dione |
| E 168 | 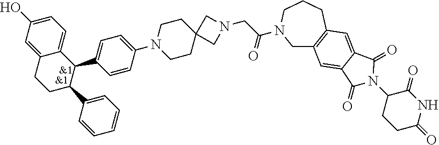 [SMILES: O=C1CCC(N2C(=O)c3cc4c(cc3C2=O)CN(C(=O)CN2CC3(CCN(c5ccc([C@@H]6c7ccc(O)cc7CC[C@@H]6c6ccccc6)cc5)CC3)C2)CCC4)C(=O)N1] | rac-2-(2,6-dioxopiperidin-3-yl)-6-(2-(7- (4-((1R,2S)-6-hydroxy-2-phenyl- 1,2,3,4-tetrahydronaphthalen-1- yl)phenyl)-2,7-diazaspiro[3.5]nonan-2- yl)acetyl)-6,7,8,9-tetrahydroazepino[3,4- f]isoindole-1,3(2H,5H)-dione |
| E 169 | 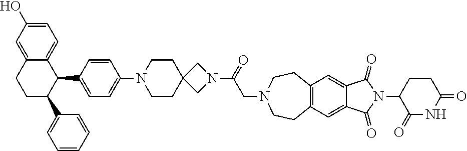 [SMILES: O=C1CCC(N2C(=O)c3cc4c(cc3C2=O)CCN(CC(=O)N2CC3(CCN(c5ccc([C@@H]6c7ccc(O)cc7CC[C@@H]6c6ccccc6)cc5)CC3)C2)CC4)C(=O)N1] | 2-(2,6-dioxopiperidin-3-yl)-7-(2-(7-(4- ((1R,2S)-6-hydroxy-2-phenyl-1,2,3,4- tetrahydronaphthalen-1-yl)phenyl)-2,7- diazaspiro[3.5]nonan-2-yl)-2-oxoethyl)- 6,7,8,9-tetrahydroazepino[4,5- f]isoindole-1,3(2H,5H)-dione |
| E 170 | 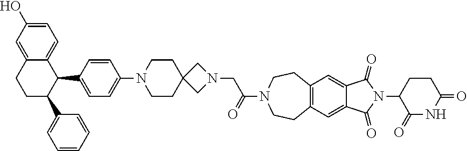 [SMILES: O=C1CCC(N2C(=O)c3cc4c(cc3C2=O)CCN(C(=O)CN2CC3(CCN(c5ccc([C@@H]6c7ccc(O)cc7CC[C@@H]6c6ccccc6)cc5)CC3)C2)CC4)C(=O)N1] | 2-(2,6-dioxopiperidin-3-yl)-7-(2-(7-(4- ((1R,2S)-6-hydroxy-2-phenyl-1,2,3,4- tetrahydronaphthalen-1-yl)phenyl)-2,7- diazaspiro[3.5]nonan-2-yl)acetyl)- 6,7,8,9-tetrahydroazepino[4,5- f]isoindole-1,3(2H,5H)-dione |
| E 171 | 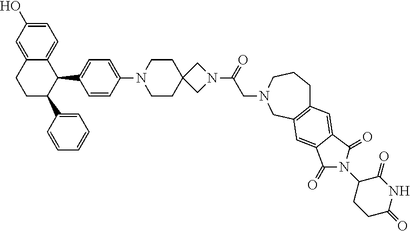 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)CCC4)C(=O)N1] | 2-(2,6-dioxopiperidin-3-yl)-6-(2-(7-(4- ((1R,2S)-6-hydroxy-2-phenyl-1,2,3,4- tetrahydronaphthalen-1-yl)phenyl)-2,7- diazaspiro[3.5]nonan-2-yl)-2-oxoethyl)- 6,7,8,9-tetrahydroazepino[3,4- f]isoindole-1,3(2H,5H)-dione |
| E172 | 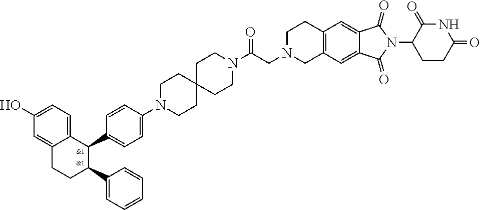 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)CC4)C(=O)N1] | rac-2-(2,6-dioxopiperidin-3-yl)-6-(2-(9- (4-((1R,2S)-6-hydroxy-2-phenyl- 1,2,3,4-tetrahydronaphthalen-1- yl)phenyl)-3,9-diazaspiro[5.5]undecan- 3-yl)-2-oxoethyl)-5,6,7,8-tetrahydro-1H- pyrrolo[3,4-g]isoquinoline-1,3(2H)- dione |
| E 173 | 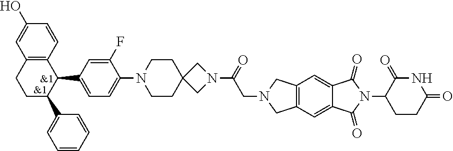 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5F)CC3)C2)C4)C(=O)N1] | Rac-2-(2,6-dioxopiperidin-3-yl)-6-(2-(7- (2-fluoro-4-((1R,2S)-6-hydroxy-2- phenyl-1,2,3,4-tetrahydronaphthalen-1- yl)phenyl)-2,7-diazaspiro[3.5]nonan-2- yl)-2-oxoethyl)-6,7-dihydropyrrolo[3,4- f]isoindole-1,3(2H,5H)-dione |
| E 174 | 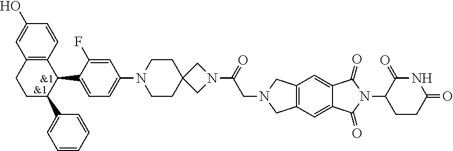 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)c(F)c5)CC3)C2)C4)C(=O)N1] | Rac-2-(2,6-dioxopiperidin-3-yl)-6-(2-(7- (3-fluoro-4-((1S,2S)-6-hydroxy-2- phenyl-1,2,3,4-tetrahydronaphthalen-1- yl)phenyl)-2,7-diazaspiro[3.5]nonan-2- yl)-2-oxoethyl)-6,7-dihydropyrrolo[3,4- f]isoindole-1,3(2H,5H)-dione |
| E 175 | 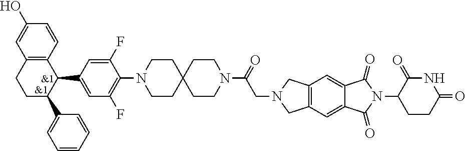 [SMILES: O=C1CCC(N2C(=O)c3cc4c(cc3C2=O)CN(CC(=O)N2CCC3(CC2)CCN(c2c(F)cc([C@@H]5c6ccc(O)cc6CC[C@@H]5c5ccccc5)cc2F)CC3)C4)C(=O)N1] | Rac-6-(2-(9-(2,6-difluoro-4-((1R,2S)-6- hydroxy-2-phenyl-1,2,3,4- tetrahydronaphthalen-1-yl)phenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 176 | 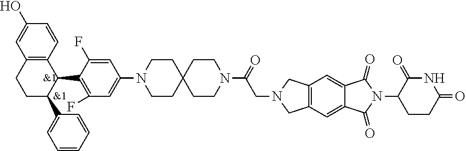 [SMILES: O=C1CCC(N2C(=O)c3cc4c(cc3C2=O)CN(CC(=O)N2CCC3(CC2)CCN(c2cc(F)c([C@@H]5c6ccc(O)cc6CC[C@@H]5c5ccccc5)c(F)c2)CC3)C4)C(=O)N1] | Rac-6-(2-(9-(3,5-difluoro-4-((1S,2S)-6- hydroxy-2-phenyl-1,2,3,4- tetrahydronaphthalen-1-yl)phenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 177 | 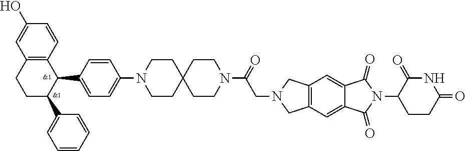 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | Rac-6-(2-(9-(4-((1R,2R)-2-cyclohexyl- 6-hydroxy-1,2,3,4- tetrahydronaphthalen-1-yl)phenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 178 | 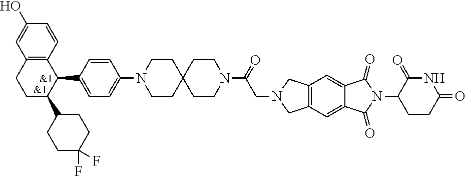 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5C5CCC(F)(F)CC5)cc2)CC3)C4)C(=O)N1] | Rac-6-(2-(9-(4-((1R,2R)-2-(4,4- difluorocyclohexyl)-6-hydroxy-1,2,3,4- tetrahydronaphthalen-1-yl)phenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 179 | 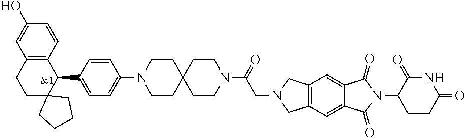 [SMILES: O=C1CCC(N2C(=O)c3cc4c(cc3C2=O)CN(CC(=O)N2CCC3(CC2)CCN(c2ccc([C@@H]5c6ccc(O)cc6CCC56CCCC6)cc2)CC3)C4)C(=O)N1] | Rac-2-(2,6-dioxopiperidin-3-yl)-6-(2-(9- (4-((R)-6′-hydroxy-3′,4′-dihydro-1′H- spiro[cyclopentane-1,2′-naphthalen]-1′- yl)phenyl)-3,9-diazaspiro[5.5]undecan- 3-yl)-2-oxoethyl)-6,7- dihydropyrrolo[3,4-f]isoindole- 1,3(2H,5H)-dione |
| E 180 | 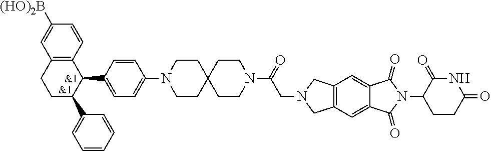 [SMILES: O=C1CCC(N2C(=O)c3cc4c(cc3C2=O)CN(CC(=O)N2CCC3(CC2)CCN(c2ccc([C@@H]5c6ccc(B(O)O)cc6CC[C@@H]5c5ccccc5)cc2)CC3)C4)C(=O)N1] | Rac-((5R,6S)-5-(4-(9-(2-(6-(2,6- dioxopiperidin-3-yl)-5,7-dioxo-3,5,6,7- tetrahydropyrrolo[3,4-f]isoindol-2(1H)- yl)acetyl)-3,9-diazaspiro[5.5]undecan-3- yl)phenyl)-6-phenyl-5,6,7,8- tetrahydronaphthalen-2-yl)boronic acid |
| E 181 | 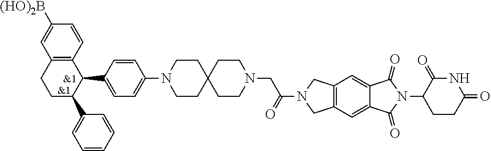 [SMILES: O=C1CCC(N2C(=O)c3cc4c(cc3C2=O)CN(C(=O)CN2CCC3(CC2)CCN(c2ccc([C@@H]5c6ccc(B(O)O)cc6CC[C@@H]5c5ccccc5)cc2)CC3)C4)C(=O)N1] | Rac-((5R,6S)-5-(4-(9-(2-(6-(2,6- dioxopiperidin-3-yl)-5,7-dioxo-3,5,6,7- tetrahydropyrrolo[3,4-f]isoindol-2(1H)- yl)-2-oxoethyl)-3,9- diazaspiro[5.5]undecan-3-yl)phenyl)-6- phenyl-5,6,7,8-tetrahydronaphthalen-2- yl)boronic acid |
| E 182 | 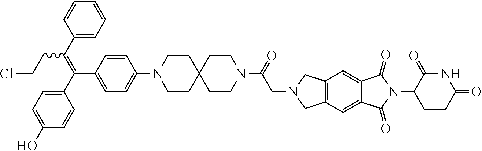 [SMILES: O=C1CCC(N2C(=O)c3cc4c(cc3C2=O)CN(CC(=O)N2CCC3(CC2)CCN(c2ccc(C(=C(CCCl)c5ccccc5)c5ccc(O)cc5)cc2)CC3)C4)C(=O)N1] | 6-(2-(9-(4-(4-chloro-1-(4- hydroxyphenyl)-2-phenylbut-1-en-1- yl)phenyl)-3,9-diazaspiro[5.5]undecan- 3-yl)-2-oxoethyl)-2-(2,6-dioxopiperidin- 3-yl)-6,7-dihydropyrrolo[3,4- f]isoindole-1,3(2H,5H)-dione |
| E 183 | 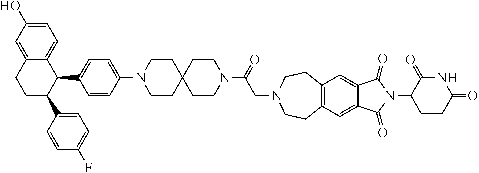 [SMILES: O=C1CCC(N2C(=O)c3cc4c(cc3C2=O)CCN(CC(=O)N2CCC3(CC2)CCN(c2ccc([C@@H]5c6ccc(O)cc6CC[C@@H]5c5ccc(F)cc5)cc2)CC3)CC4)C(=O)N1] | 2-(2,6-dioxopiperidin-3-yl)-7-(2-(9-(4- ((1R,2S)-2-(4-fluorophenyl)-6-hydroxy- 1,2,3,4-tetrahydronaphthalen-1- yl)phenyl)-3,9-diazaspiro[5.5]undecan- 3-yl)-2-oxoethyl)-6,7,8,9- tetrahydroazepino[4,5-f]isoindole- 1,3(2H,5H)-dione |
| E 184 | 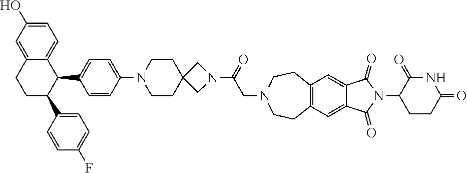 [SMILES: O=C1CCC(N2C(=O)c3cc4c(cc3C2=O)CCN(CC(=O)N2CC3(CCN(c5ccc([C@@H]6c7ccc(O)cc7CC[C@@H]6c6ccc(F)cc6)cc5)CC3)C2)CC4)C(=O)N1] | 2-(2,6-dioxopiperidin-3-yl)-7-(2-(7-(4- ((1R,2S)-2-(4-fluorophenyl)-6-hydroxy- 1,2,3,4-tetrahydronaphthalen-1- yl)phenyl)-2,7-diazaspiro[3.5]nonan-2- yl)-2-oxoethyl)-6,7,8,9- tetrahydroazepino[4,5-f]isoindole- 1,3(2H,5H)-dione |
| E 185 | 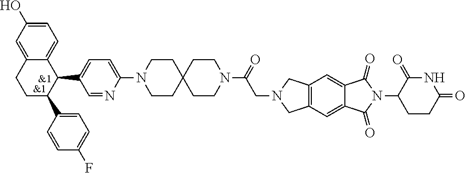 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccc(F)cc5)cn2)CC3)C4)C(=O)N1] | Rac-2-(2,6-dioxopiperidin-3-yl)-6-(2-(9- (5-((1S,2S)-2-(4-fluorophenyl)-6- hydroxy-1,2,3,4-tetrahydronaphthalen- 1-yl)pyridin-2-yl)-3,9- diazaspiro[5.5]undecan-3-yl)-2- oxoethyl)-6,7-dihydropyrrolo[3,4- f]isoindole-1,3(2H,5H)-dione |
| E 186 | 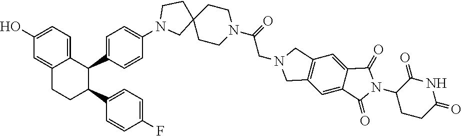 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccc(F)cc5)cc2)C3)C4)C(=O)N1] | 2-(2,6-dioxopiperidin-3-yl)-6-(2-(2-(4- ((1R,2S)-2-(4-fluorophenyl)-6-hydroxy- 1,2,3,4-tetrahydronaphthalen-1- yl)phenyl)-2,8-diazaspiro[4.5]decan-8- yl)-2-oxoethyl)-6,7-dihydropyrrolo[3,4- f]isoindole-1,3(2H,5H)-dione |
| E 187 | 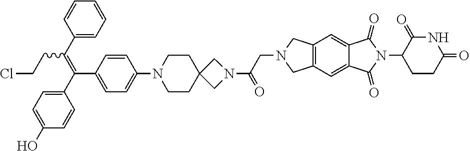 [SMILES: O=C1CCC(N2C(=O)c3cc4c(cc3C2=O)CN(CC(=O)N2CC3(CCN(c5ccc(C(=C(CCCl)c6ccccc6)c6ccc(O)cc6)cc5)CC3)C2)C4)C(=O)N1] | 6-(2-(7-(4-(4-chloro-1-(4- hydroxyphenyl)-2-phenylbut-1-en-1- yl)phenyl)-2,7-diazaspiro[3.5]nonan-2- yl)-2-oxoethyl)-2-(2,6-dioxopiperidin-3- yl)-6,7-dihydropyrrolo[3,4-f]isoindole- 1,3(2H,5H)-dione |
| E 188 | 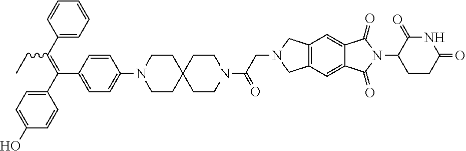 [SMILES: CCC(=C(c1ccc(O)cc1)c1ccc(N2CCC3(CCN(C(=O)CN4Cc5cc6c(cc5C4)C(=O)N(C4CCC(=O)NC4=O)C6=O)CC3)CC2)cc1)c1ccccc1] | 2-(2,6-dioxopiperidin-3-yl)-6-(2-(9-(4- (1-(4-hydroxyphenyl)-2-phenylbut-1-en- 1-yl)phenyl)-3,9- diazaspiro[5.5]undecan-3-yl)-2- oxoethyl)-6,7-dihydropyrrolo[3,4- f]isoindole-1,3(2H,5H)-dione |
| E 189 | 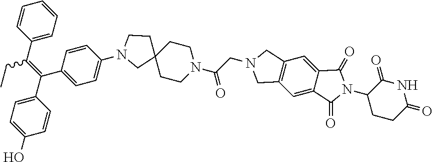 [SMILES: CCC(=C(c1ccc(O)cc1)c1ccc(N2CCC3(CCN(C(=O)CN4Cc5cc6c(cc5C4)C(=O)N(C4CCC(=O)NC4=O)C6=O)CC3)C2)cc1)c1ccccc1] | 2-(2,6-dioxopiperidin-3-yl)-6-(2-(2-(4- (1-(4-hydroxyphenyl)-2-phenylbut-1-en- 1-yl)phenyl)-2,8-diazaspiro[4.5]decan- 8-yl)-2-oxoethyl)-6,7- dihydropyrrolo[3,4-f]isoindole- 1,3(2H,5H)-dione |
| E 190 | 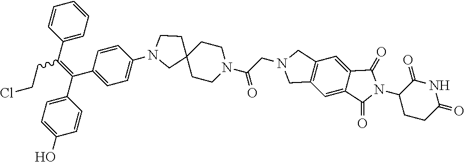 [SMILES: O=C1CCC(N2C(=O)c3cc4c(cc3C2=O)CN(CC(=O)N2CCC3(CC2)CCN(c2ccc(C(=C(CCCl)c5ccccc5)c5ccc(O)cc5)cc2)C3)C4)C(=O)N1] | 6-(2-(2-(4-(4-chloro-1-(4- hydroxyphenyl)-2-phenylbut-1-en-1- yl)phenyl)-2,8-diazaspiro[4.5]decan-8- yl)-2-oxoethyl)-2-(2,6-dioxopiperidin-3- yl)-6,7-dihydropyrrolo[3,4-f]isoindole- 1,3(2H,5H)-dione |
| E 191 | 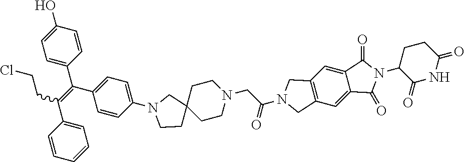 [SMILES: O=C1CCC(N2C(=O)c3cc4c(cc3C2=O)CN(C(=O)CN2CCC3(CC2)CCN(c2ccc(C(=C(CCCl)c5ccccc5)c5ccc(O)cc5)cc2)C3)C4)C(=O)N1] | 6-(2-(2-(4-(4-chloro-1-(4- hydroxyphenyl)-2-phenylbut-1-en-1- yl)phenyl)-2,8-diazaspiro[4.5]decan-8- yl)acetyl)-2-(2,6-dioxopiperidin-3-yl)- 6,7-dihydropyrrolo[3,4-f]isoindole- 1,3(2H,5H)-dione |
| E 192 | 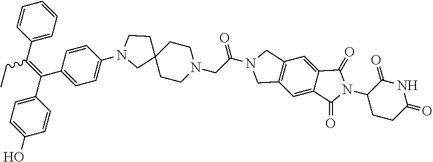 [SMILES: CCC(=C(c1ccc(O)cc1)c1ccc(N2CCC3(CCN(CC(=O)N4Cc5cc6c(cc5C4)C(=O)N(C4CCC(=O)NC4=O)C6=O)CC3)C2)cc1)c1ccccc1] | 2-(2,6-dioxopiperidin-3-yl)-6-(2-(2-(4- (1-(4-hydroxyphenyl)-2-phenylbut-1-en- 1-yl)phenyl)-2,8-diazaspiro[4.5]decan- 8-yl)acetyl)-6,7-dihydropyrrolo[3,4- f]isoindole-1,3(2H,5H)-dione |
| E 193 | 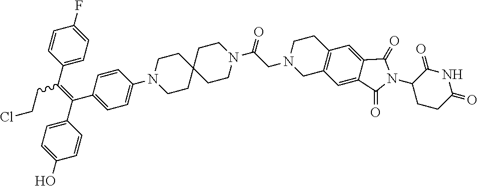 [SMILES: O=C1CCC(N2C(=O)c3cc4c(cc3C2=O)CN(CC(=O)N2CCC3(CC2)CCN(c2ccc(C(=C(CCCl)c5ccc(F)cc5)c5ccc(O)cc5)cc2)CC3)CC4)C(=O)N1] | 6-(2-(9-(4-(4-chloro-2-(4-fluorophenyl)- 1-(4-hydroxyphenyl)but-1-en-1- yl)phenyl)-3,9-diazaspiro[5.5]undecan- 3-yl)-2-oxoethyl)-2-(2,6-dioxopiperidin- 3-yl)-5,6,7,8-tetrahydro-1H-pyrrolo[3,4- glisoquinoline-1,3(2H)-dione |
| E 194 | 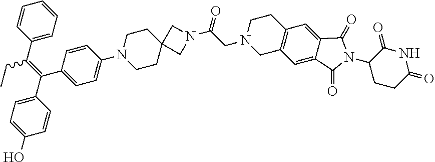 [SMILES: CCC(=C(c1ccc(O)cc1)c1ccc(N2CCC3(CC2)CN(C(=O)CN2CCc4cc5c(cc4C2)C(=O)N(C2CCC(=O)NC2=O)C5=O)C3)cc1)c1ccccc1] | 2-(2,6-dioxopiperidin-3-yl)-6-(2-(7-(4- (1-(4-hydroxyphenyl)-2-phenylbut-1-en- 1-yl)phenyl)-2,7-diazaspiro[3.5]nonan- 2-yl)-2-oxoethyl)-5,6,7,8-tetrahydro-1H- pyrrolo[3,4-g]isoquinoline-1,3(2H)- dione |
| E 195 | 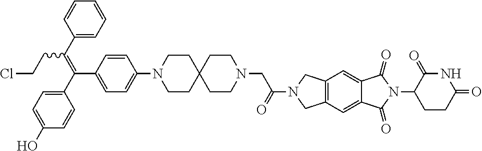 [SMILES: O=C1CCC(N2C(=O)c3cc4c(cc3C2=O)CN(C(=O)CN2CCC3(CC2)CCN(c2ccc(C(=C(CCCl)c5ccccc5)c5ccc(O)cc5)cc2)CC3)C4)C(=O)N1] | 6-(2-(9-(4-(4-chloro-1-(4- hydroxyphenyl)-2-phenylbut-1-en-1- yl)phenyl)-3,9-diazaspiro[5.5]undecan- 3-yl)acetyl)-2-(2,6-dioxopiperidin-3-yl)- 6,7-dihydropyrrolo[3,4-f]isoindole- 1,3(2H,5H)-dione |
| E 196 | 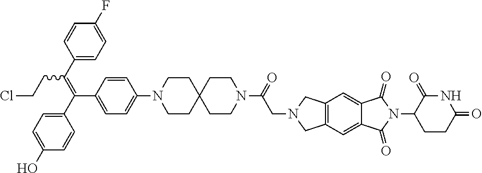 [SMILES: O=C1CCC(N2C(=O)c3cc4c(cc3C2=O)CN(CC(=O)N2CCC3(CC2)CCN(c2ccc(C(=C(CCCl)c5ccc(F)cc5)c5ccc(O)cc5)cc2)CC3)C4)C(=O)N1] | 6-(2-(9-(4-(4-chloro-2-(4-fluorophenyl)- 1-(4-hydroxyphenyl)but-1-en-1- yl)phenyl)-3,9-diazaspiro[5.5]undecan- 3-yl)-2-oxoethyl)-2-(2,6-dioxopiperidin- 3-yl)-6,7-dihydropyrrolo[3,4- f]isoindole-1,3(2H,5H)-dione |
| E 197 | 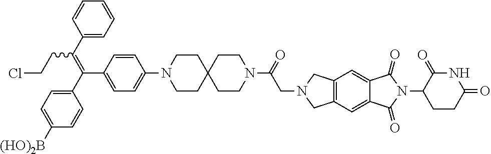 [SMILES: O=C1CCC(N2C(=O)c3cc4c(cc3C2=O)CN(CC(=O)N2CCC3(CC2)CCN(c2ccc(C(=C(CCCl)c5ccccc5)c5ccc(B(O)O)cc5)cc2)CC3)C4)C(=O)N1] | (4-(4-chloro-1-(4-(9-(2-(6-(2,6- dioxopiperidin-3-yl)-5,7-dioxo-3,5,6,7- tetrahydropyrrolo[3,4-f]isoindol-2(1H)- yl)acetyl)-3,9-diazaspiro[5.5]undecan-3- yl)phenyl)-2-phenylbut-1-en-1- yl)phenyl)boronic acid |
| E 198 | 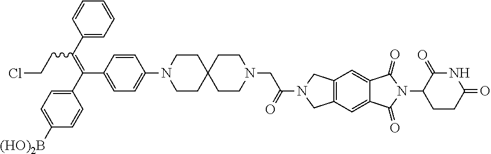 [SMILES: O=C1CCC(N2C(=O)c3cc4c(cc3C2=O)CN(C(=O)CN2CCC3(CC2)CCN(c2ccc(C(=C(CCCl)c5ccccc5)c5ccc(B(O)O)cc5)cc2)CC3)C4)C(=O)N1] | (4-(4-chloro-1-(4-(9-(2-(6-(2,6- dioxopiperidin-3-yl)-5,7-dioxo-3,5,6,7- tetrahydropyrrolo[3,4-f]isoindol-2(1H)- yl)-2-oxoethyl)-3,9- diazaspiro[5.5]undecan-3-yl)phenyl)-2- phenylbut-1-en-1-yl)phenyl)boronic acid |
| E 199 | 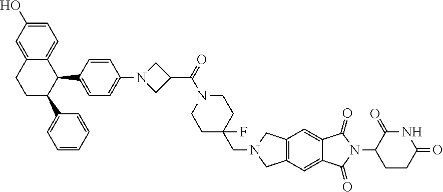 [SMILES: O=C1CCC(N2C(=O)c3cc4c(cc3C2=O)CN(CC2(F)CCN(C(=O)C3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-fluoro- 1-(1-(4-((1R,2S)-6-hydroxy-2-phenyl- 1,2,3,4-tetrahydronaphthalen-1- yl)phenyl)azetidine-3- carbonyl)piperidin-4-yl)methyl)-6,7- dihydropyrrolo[3,4-f]isoindole- 1,3(2H,5H)-dione |
| E 200 | 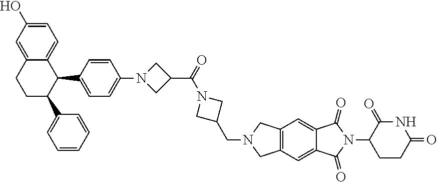 [SMILES: O=C1CCC(N2C(=O)c3cc4c(cc3C2=O)CN(CC2CN(C(=O)C3CN(c5ccc([C@@H]6c7ccc(O)cc7CC[C@@H]6c6ccccc6)cc5)C3)C2)C4)C(=O)N1] | 2-(2,6-dioxopiperidin-3-yl)-6-((1-(1-(4- ((1R,2S)-6-hydroxy-2-phenyl-1,2,3,4- tetrahydronaphthalen-1- yl)phenyl)azetidine-3-carbonyl)azetidin- 3-yl)methyl)-6,7-dihydropyrrolo[3,4- f]isoindole-1,3(2H,5H)-dione |
| E 201 | 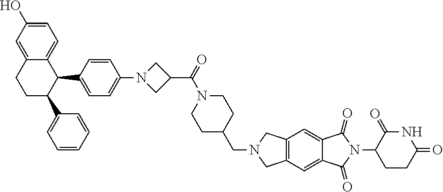 [SMILES: O=C1CCC(N2C(=O)c3cc4c(cc3C2=O)CN(CC2CCN(C(=O)C3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-(1-(4- ((1R,2S)-6-hydroxy-2-phenyl-1,2,3,4- tetrahydronaphthalen-1- yl)phenyl)azetidine-3- carbonyl)piperidin-4-yl)methyl)-6,7- dihydropyrrolo[3,4-f]isoindole- 1,3(2H,5H)-dione |
| E 202 | 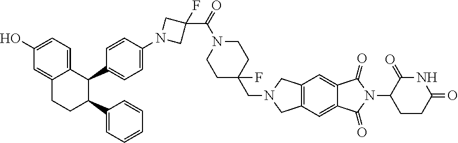 [SMILES: O=C1CCC(N2C(=O)c3cc4c(cc3C2=O)CN(CC2(F)CCN(C(=O)C3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-fluoro- 1-(3-fluoro-1-(4-((1R,2S)-6-hydroxy-2- phenyl-1,2,3,4-tetrahydronaphthalen-1- yl)phenyl)azetidine-3- carbonyl)piperidin-4-yl)methyl)-6,7- dihydropyrrolo[3,4-f]isoindole- 1,3(2H,5H)-dione |
| E 203 | 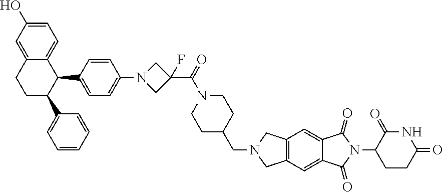 [SMILES: O=C1CCC(N2C(=O)c3cc4c(cc3C2=O)CN(CC2CCN(C(=O)C3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-(3- fluoro-1-(4-((1R,2S)-6-hydroxy-2- phenyl-1,2,3,4-tetrahydronaphthalen-1- yl)phenyl)azetidine-3- carbonyl)piperidin-4-yl)methyl)-6,7- dihydropyrrolo[3,4-f]isoindole- 1,3(2H,5H)-dione |
| E 204 | 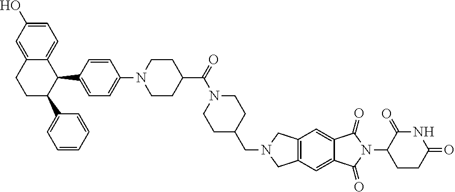 [SMILES: O=C1CCC(N2C(=O)c3cc4c(cc3C2=O)CN(CC2CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-(1-(4- ((1R,2S)-6-hydroxy-2-phenyl-1,2,3,4- tetrahydronaphthalen-1- yl)phenyl)piperidine-4- carbonyl)piperidin-4-yl)methyl)-6,7- dihydropyrrolo[3,4-f]isoindole- 1,3(2H,5H)-dione |
| E 205 | 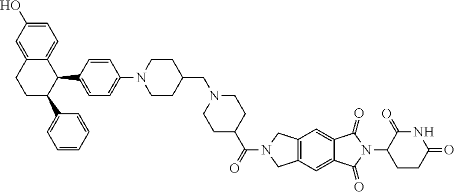 [SMILES: O=C1CCC(N2C(=O)c3cc4c(cc3C2=O)CN(C(=O)C2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)methyl)piperidine-4-carbonyl)-6,7- dihydropyrrolo[3,4-f]isoindole- 1,3(2H,5H)-dione |
| E 206 | 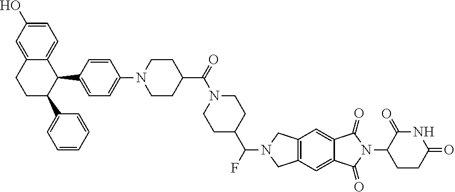 [SMILES: O=C1CCC(N2C(=O)c3cc4c(cc3C2=O)CN(C(F)C2CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((4-fluoro- 1-(1-(4-((1R,2S)-6-hydroxy-2-phenyl- 1,2,3,4-tetrahydronaphthalen-1- yl)phenyl)piperidine-4- carbonyl)piperidin-4-yl)methyl)-6,7- dihydropyrrolo[3,4-f]isoindole- 1,3(2H,5H)-dione |
| E 207 | 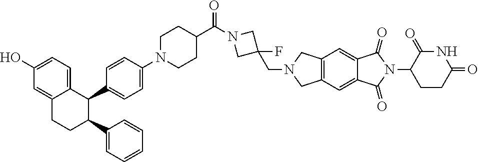 [SMILES: O=C1CCC(N2C(=O)c3cc4c(cc3C2=O)CN(CC2(F)CN(C(=O)C3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((3-fluoro- 1-(1-(4-((1R,2S)-6-hydroxy-2-phenyl- 1,2,3,4-tetrahydronaphthalen-1- yl)phenyl)piperidine-4- carbonyl)azetidin-3-yl)methyl)-6,7- dihydropyrrolo[3,4-f]isoindole- 1,3(2H,5H)-dione |
| E 208 | 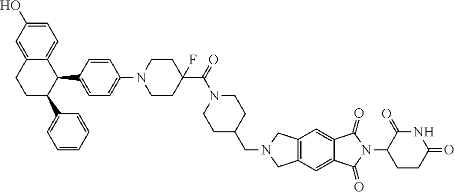 [SMILES: O=C1CCC(N2C(=O)c3cc4c(cc3C2=O)CN(CC2CCN(C(=O)C3(F)CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-(4- fluoro-1-(4-((1R,2S)-6-hydroxy-2- phenyl-1,2,3,4-tetrahydronaphthalen-1- yl)phenyl)piperidine-4- carbonyl)piperidin-4-yl)methyl)-6,7- dihydropyrrolo[3,4-f]isoindole- 1,3(2H,5H)-dione |
| E 209 | 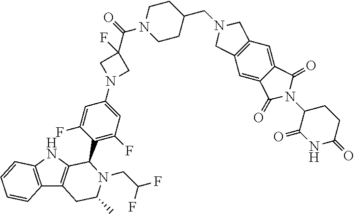 [SMILES: C[C@@H]1Cc2c([nH]c3ccccc23)[C@@H](c2c(F)cc(N3CC(F)(C(=O)N4CCC(CN5Cc6cc7c(cc6C5)C(=O)N(C5CCC(=O)NC5=O)C7=O)CC4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)-3-fluoroazetidine-3- carbonyl)piperidin-4-yl)methyl)-2-(2,6- dioxopiperidin-3-yl)-6,7- dihydropyrrolo[3,4-f]isoindole- 1,3(2H,5H)-dione |
| E 210 | 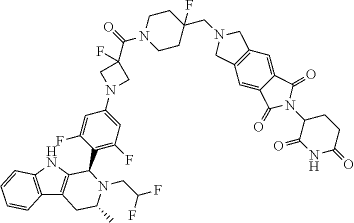 [SMILES: C[C@@H]1Cc2c([nH]c3ccccc23)[C@@H](c2c(F)cc(N3CC(F)(C(=O)N4CCC(F)(CN5Cc6cc7c(cc6C5)C(=O)N(C5CCC(=O)NC5=O)C7=O)CC4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)-3-fluoroazetidine-3- carbonyl)-4-fluoropiperidin-4- yl)methyl)-2-(2,6-dioxopiperidin-3-yl)- 6,7-dihydropyrrolo[3,4-f]isoindole- 1,3(2H,5H)-dione |
| E 211 | 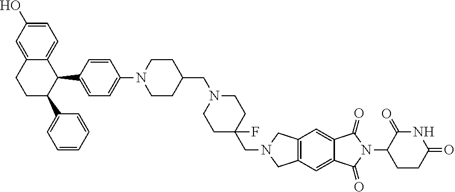 [SMILES: O=C1CCC(N2C(=O)c3cc4c(cc3C2=O)CN(CC2(F)CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((4-fluoro- 1-((1-(4-((1R,2S)-6-hydroxy-2-phenyl- 1,2,3,4-tetrahydronaphthalen-1- yl)phenyl)piperidin-4- yl)methyl)piperidin-4-yl)methyl)-6,7- dihydropyrrolo[3,4-f]isoindole- 1,3(2H,5H)-dione |
| E 212 | 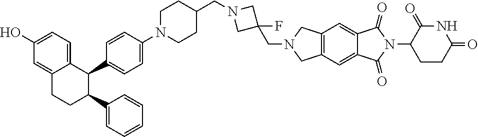 [SMILES: O=C1CCC(N2C(=O)c3cc4c(cc3C2=O)CN(CC2(F)CN(CC3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((3-fluoro- 1-((1-(4-((1R,2S)-6-hydroxy-2-phenyl- 1,2,3,4-tetrahydronaphthalen-1- yl)phenyl)piperidin-4- yl)methyl)azetidin-3-yl)methyl)-6,7- dihydropyrrolo[3,4-f]isoindole- 1,3(2H,5H)-dione |
| E 213 | 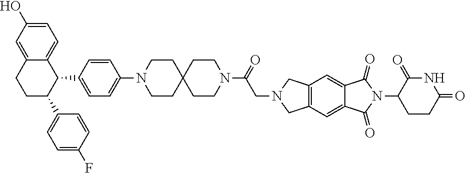 [SMILES: O=C1CCC(N2C(=O)c3cc4c(cc3C2=O)CN(CC(=O)N2CCC3(CC2)CCN(c2ccc([C@H]5c6ccc(O)cc6CC[C@H]5c5ccc(F)cc5)cc2)CC3)C4)C(=O)N1] | 2-(2,6-dioxopiperidin-3-yl)-6-(2-(9-(4- ((1S,2R)-2-(4-fluorophenyl)-6-hydroxy- 1,2,3,4-tetrahydronaphthalen-1- yl)phenyl)-3,9-diazaspiro[5.5]undecan- 3-yl)-2-oxoethyl)-6,7- dihydropyrrolo[3,4-f]isoindole- 1,3(2H,5H)-dione |
| E 214 | 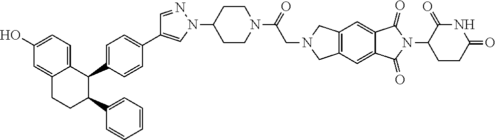 [SMILES: O=C1CCC(N2C(=O)c3cc4c(cc3C2=O)CN(CC(=O)N2CCC(n3cc(-c5ccc([C@@H]6c7ccc(O)cc7CC[C@@H]6c6ccccc6)cc5)cn3)CC2)C4)C(=O)N1] | 2-(2,6-dioxopiperidin-3-yl)-6-(2-(4-(4- (4-((1R,2S)-6-hydroxy-2-phenyl- 1,2,3,4-tetrahydronaphthalen-1- yl)phenyl)-1H-pyrazol-1-yl)piperidin-1- yl)-2-oxoethyl)-6,7-dihydropyrrolo[3,4- f]isoindole-1,3(2H,5H)-dione |
| E 215 | 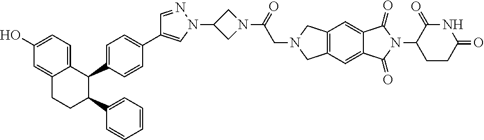 [SMILES: O=C1CCC(N2C(=O)c3cc4c(cc3C2=O)CN(CC(=O)N2CC(n3cc(-c5ccc([C@@H]6c7ccc(O)cc7CC[C@@H]6c6ccccc6)cc5)cn3)C2)C4)C(=O)N1] | 2-(2,6-dioxopiperidin-3-yl)-6-(2-(3-(4- (4-((1R,2S)-6-hydroxy-2-phenyl- 1,2,3,4-tetrahydronaphthalen-1- yl)phenyl)-1H-pyrazol-1-yl)azetidin-1- yl)-2-oxoethyl)-6,7-dihydropyrrolo[3,4- f]isoindole-1,3(2H,5H)-dione |
| E 216 | 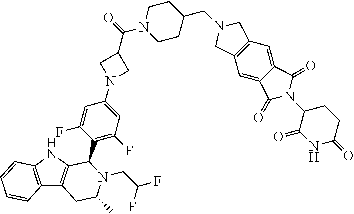 [SMILES: C[C@@H]1Cc2c([nH]c3ccccc23)[C@@H](c2c(F)cc(N3CC(C(=O)N4CCC(CN5Cc6cc7c(cc6C5)C(=O)N(C5CCC(=O)NC5=O)C7=O)CC4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)piperidin-4-yl)methyl)-2-(2,6- dioxopiperidin-3-yl)-6,7- dihydropyrrolo[3,4-f]isoindole- 1,3(2H,5H)-dione |
| E 217 | 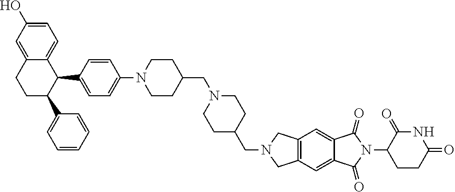 [SMILES: O=C1CCC(N2C(=O)c3cc4c(cc3C2=O)CN(CC2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-((1-(4- ((1R,2S)-6-hydroxy-2-phenyl-1,2,3,4- tetrahydronaphthalen-1- yl)phenyl)piperidin-4- yl)methyl)piperidin-4-yl)methyl)-6,7- dihydropyrrolo[3,4-f]isoindole- 1,3(2H,5H)-dione |
| E 218 | 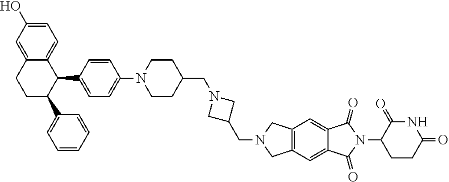 [SMILES: O=C1CCC(N2C(=O)c3cc4c(cc3C2=O)CN(CC2CN(CC3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((1-((1-(4- ((1R,2S)-6-hydroxy-2-phenyl-1,2,3,4- tetrahydronaphthalen-1- yl)phenyl)piperidin-4- yl)methyl)azetidin-3-yl)methyl)-6,7- dihydropyrrolo[3,4-f]isoindole- 1,3(2H,5H)-dione |
| E 219 | 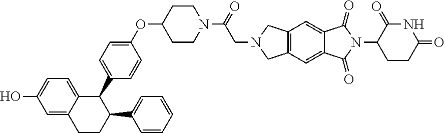 [SMILES: O=C1CCC(N2C(=O)c3cc4c(cc3C2=O)CN(CC(=O)N2CCC(Oc3ccc([C@@H]5c6ccc(O)cc6CC[C@@H]5c5ccccc5)cc3)CC2)C4)C(=O)N1] | 2-(2,6-dioxopiperidin-3-yl)-6-(2-(4-(4- ((1R,2S)-6-hydroxy-2-phenyl-1,2,3,4- tetrahydronaphthalen-1- yl)phenoxy)piperidin-1-yl)-2-oxoethyl)- 6,7-dihydropyrrolo[3,4-f]isoindole- 1,3(2H,5H)-dione |
| E 220 | 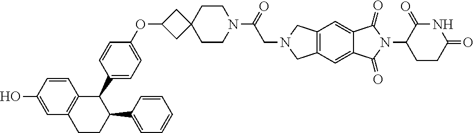 [SMILES: O=C1CCC(N2C(=O)c3cc4c(cc3C2=O)CN(CC(=O)N2CCC3(CC2)CC(Oc2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4- ((1R,2S)-6-hydroxy-2-phenyl-1,2,3,4- tetrahydronaphthalen-1-yl)phenoxy)-7- azaspiro[3.5]nonan-7-yl)-2-oxoethyl)- 6,7-dihydropyrrolo[3,4-f]isoindole- 1,3(2H,5H)-dione |
| E 221 | 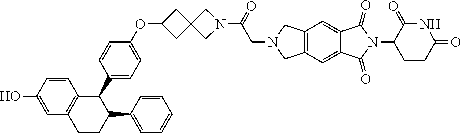 | 2-(2,6-dioxopiperidin-3-yl)-6-(2-(6-(4- ((1R,2R)-6-hydroxy-2-phenyl-1,2,3,4- tetrahydronaphthalen-1-yl)phenoxy)-2- azaspiro[3.3]heptan-2-yl)-2-oxoethyl)- 6,7-dihydropyrrolo[3,4-f]isoindole- 1,3(2H,5H)-dione |
| E 222 | 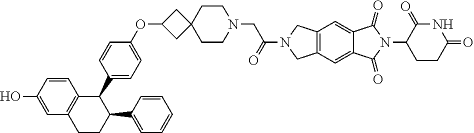 [SMILES: O=C1CCC(N2C(=O)c3cc4c(cc3C2=O)CN(C(=O)CN2CCC3(CC2)CC(Oc2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4- ((1R,2S)-6-hydroxy-2-phenyl-1,2,3,4- tetrahydronaphthalen-1-yl)phenoxy)-7- azaspiro[3.5]nonan-7-yl)acetyl)-6,7- dihydropyrrolo[3,4-f]isoindole- 1,3(2H,5H)-dione |
| E 223 | 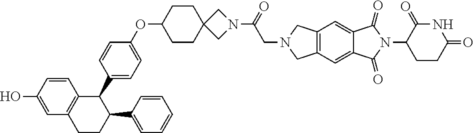 [SMILES: O=C1CCC(N2C(=O)c3cc4c(cc3C2=O)CN(CC(=O)N2CC3(CCC(Oc5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(7-(4- ((1R,2S)-6-hydroxy-2-phenyl-1,2,3,4- tetrahydronaphthalen-1-yl)phenoxy)-2- azaspiro[3.5]nonan-2-yl)-2-oxoethyl)- 6,7-dihydropyrrolo[3,4-f]isoindole- 1,3(2H,5H)-dione |
| E 224 | 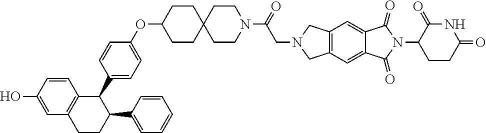 [SMILES: O=C1CCC(N2C(=O)c3cc4c(cc3C2=O)CN(CC(=O)N2CCC3(CCC(Oc5ccc([C@@H]6c7ccc(O)cc7CC[C@@H]6c6ccccc6)cc5)CC3)CC2)C4)C(=O)N1] | 2-(2,6-dioxopiperidin-3-yl)-6-(2-(9-(4- ((1R,2S)-6-hydroxy-2-phenyl-1,2,3,4- tetrahydronaphthalen-1-yl)phenoxy)-3- azaspiro[5.5]undecan-3-yl)-2-oxoethyl)- 6,7-dihydropyrrolo[3,4-f]isoindole- 1,3(2H,5H)-dione |
| E 225 | 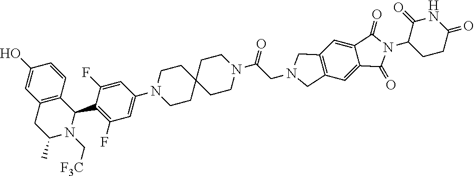 [SMILES: C[C@@H]1Cc2cc(O)ccc2[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)(F)F] | 6-(2-(9-(3,5-difluoro-4-((1S,3R)-6- hydroxy-3-methyl-2-(2,2,2- trifluoroethyl)-1,2,3,4- tetrahydroisoquinolin-1-yl)phenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 226 | 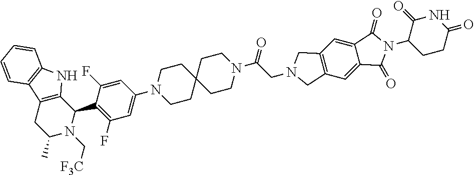 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)(F)F] | 6-(2-(9-(3,5-difluoro-4-((1R,3R)-3- methyl-2-(2,2,2-trifluoroethyl)-2,3,4,9- tetrahydro-1H-pyrido[3,4-b]indol-1- yl)phenyl)-3,9-diazaspiro[5.5]undecan- 3-yl)-2-oxoethyl)-2-(2,6-dioxopiperidin- 3-yl)-6,7-dihydropyrrolo[3,4- f]isoindole-1,3(2H,5H)-dione |
| E 227 | 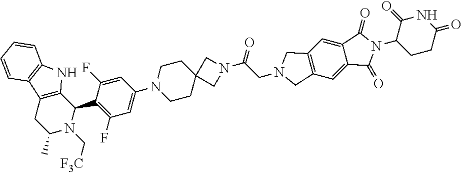 [SMILES: C[C@@H]1Cc2c([nH]c3ccccc23)[C@@H](c2c(F)cc(N3CCC4(CC3)CN(C(=O)CN3Cc5cc6c(cc5C3)C(=O)N(C3CCC(=O)NC3=O)C6=O)C4)cc2F)N1CC(F)(F)F] | 6-(2-(7-(3,5-difluoro-4-((1R,3R)-3- methyl-2-(2,2,2-trifluoroethyl)-2,3,4,9- tetrahydro-1H-pyrido[3,4-b]indol-1- yl)phenyl)-2,7-diazaspiro[3.5]nonan-2- yl)-2-oxoethyl)-2-(2,6-dioxopiperidin-3- yl)-6,7-dihydropyrrolo[3,4-f]isoindole- 1,3(2H,5H)-dione |
| E 228 | 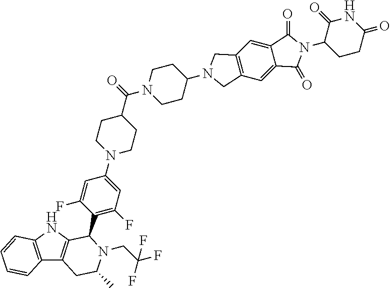 [SMILES: C[C@@H]1Cc2c([nH]c3ccccc23)[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(F)(F)F] | 6-(1-(1-(3,5-difluoro-4-((1R,3R)-3- methyl-2-(2,2,2-trifluoroethyl)-2,3,4,9- tetrahydro-1H-pyrido[3,4-b]indol-1- yl)phenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 229 | 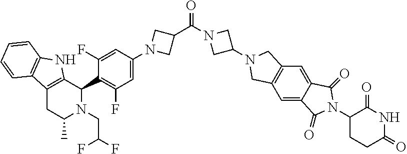 [SMILES: C[C@@H]1Cc2c([nH]c3ccccc23)[C@@H](c2c(F)cc(N3CC(C(=O)N4CC(N5Cc6cc7c(cc6C5)C(=O)N(C5CCC(=O)NC5=O)C7=O)C4)C3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)azetidin-3-yl)-2-(2,6- dioxopiperidin-3-yl)-6,7- dihydropyrrolo[3,4-f]isoindole- 1,3(2H,5H)-dione |
| E 230 | 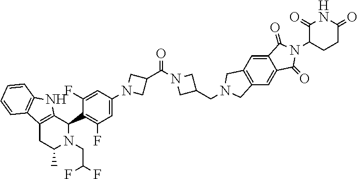 [SMILES: C[C@@H]1Cc2c([nH]c3ccccc23)[C@@H](c2c(F)cc(N3CC(C(=O)N4CC(CN5Cc6cc7c(cc6C5)C(=O)N(C5CCC(=O)NC5=O)C7=O)C4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)azetidin-3-yl)methyl)-2-(2,6- dioxopiperidin-3-yl)-6,7- dihydropyrrolo[3,4-f]isoindole- 1,3(2H,5H)-dione |
| E 231 | 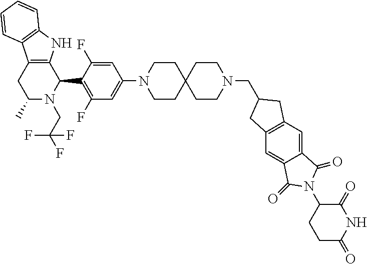 [SMILES: C[C@@H]1Cc2c([nH]c3ccccc23)[C@@H](c2c(F)cc(N3CCC4(CCN(CC5Cc6cc7c(cc6C5)C(=O)N(C5CCC(=O)NC5=O)C7=O)CC4)CC3)cc2F)N1CC(F)(F)F] | 6-((9-(3,5-difluoro-4-((1R,3R)-3- methyl-2-(2,2,2-trifluoroethyl)-2,3,4,9- tetrahydro-1H-pyrido[3,4-b ]indol-1- yl)phenyl)-3,9-diazaspiro[5.5]undecan- 3-yl)methyl)-2-(2,6-dioxopiperidin-3- yl)-6,7-dihydrocyclopenta[f]isoindole- 1,3(2H,5H)-dione |
| E 232 | 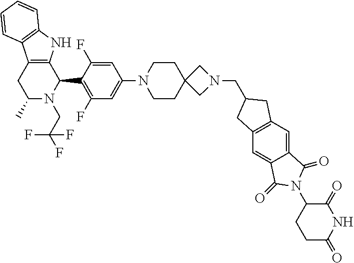 [SMILES: C[C@@H]1Cc2c([nH]c3ccccc23)[C@@H](c2c(F)cc(N3CCC4(CC3)CN(CC3Cc5cc6c(cc5C3)C(=O)N(C3CCC(=O)NC3=O)C6=O)C4)cc2F)N1CC(F)(F)F] | 6-((7-(4-((1R,3R)-2-(2,2-difluoroethyl)- 3-methyl-2,3,4,9-tetrahydro-1H- pyrido[3,4-b]indol-1-yl)-3,5- difluorophenyl)-2,7- diazaspiro[3.5]nonan-2-yl)methyl)-2- (2,6-dioxopiperidin-3-yl)-6,7- dihydrocyclopenta[f]isoindole- 1,3(2H,5H)-dione |
| E 233 | 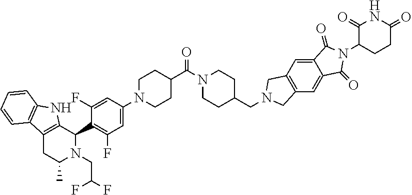 [SMILES: C[C@@H]1Cc2c([nH]c3ccccc23)[C@@H](c2c(F)cc(N3CCC(C(=O)N4CCC(CN5Cc6cc7c(cc6C5)C(=O)N(C5CCC(=O)NC5=O)C7=O)CC4)CC3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-4- carbonyl)piperidin-4-yl)methyl)-2-(2,6- dioxopiperidin-3-yl)-6,7- dihydropyrrolo[3,4-f]isoindole- 1,3(2H,5H)-dione |
| E 234 | 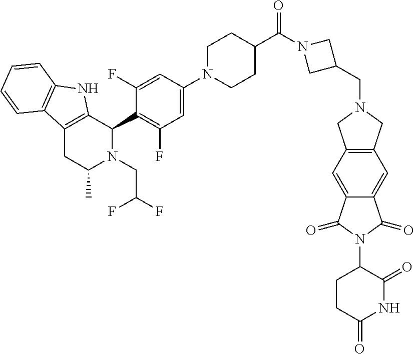 [SMILES: C[C@@H]1Cc2c([nH]c3ccccc23)[C@@H](c2c(F)cc(N3CCC(C(=O)N4CC(CN5Cc6cc7c(cc6C5)C(=O)N(C5CCC(=O)NC5=O)C7=O)C4)CC3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-4- carbonyl)azetidin-3-yl)methyl)-2-(2,6- dioxopiperidin-3-yl)-6,7- dihydropyrrolo[3,4-f]isoindole- 1,3(2H,5H)-dione |
| E 235 | 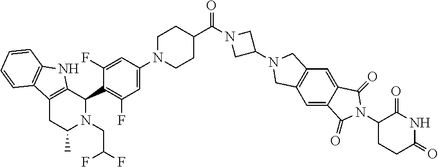 [SMILES: C[C@@H]1Cc2c([nH]c3ccccc23)[C@@H](c2c(F)cc(N3CCC(C(=O)N4CC(N5Cc6cc7c(cc6C5)C(=O)N(C5CCC(=O)NC5=O)C7=O)C4)CC3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-4- carbonyl)azetidin-3-yl)-2-(2,6- dioxopiperidin-3-yl)-6,7- dihydropyrrolo[3,4-f]isoindole- 1,3(2H,5H)-dione |
| E 236 | 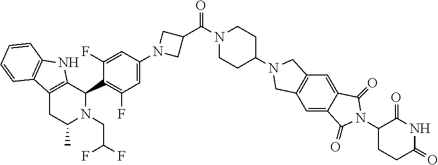 [SMILES: C[C@@H]1Cc2c([nH]c3ccccc23)[C@@H](c2c(F)cc(N3CC(C(=O)N4CCC(N5Cc6cc7c(cc6C5)C(=O)N(C5CCC(=O)NC5=O)C7=O)CC4)C3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)piperidin-4-yl)-2-(2,6- dioxopiperidin-3-yl)-6,7- dihydropyrrolo[3,4-f]isoindole- 1,3(2H,5H)-dione |
| E 237 | 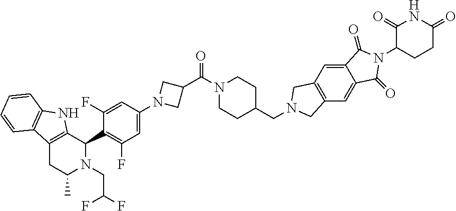 [SMILES: C[C@@H]1Cc2c([nH]c3ccccc23)[C@@H](c2c(F)cc(N3CC(C(=O)N4CCC(CN5Cc6cc7c(cc6C5)C(=O)N(C5CCC(=O)NC5=O)C7=O)CC4)C3)cc2F)N1CC(F)F] | 6-((1-(1-(4-((1R,3R)-2-(2,2- difluoroethyl)-3-methyl-2,3,4,9- tetrahydro-1H-pyrido[3,4-b]indol-1-yl)- 3,5-difluorophenyl)azetidine-3- carbonyl)piperidin-4-yl)methyl)-2-(2,6- dioxopiperidin-3-yl)-6,7- dihydropyrrolo[3,4-f]isoindole- 1,3(2H,5H)-dione |
| E 238 | 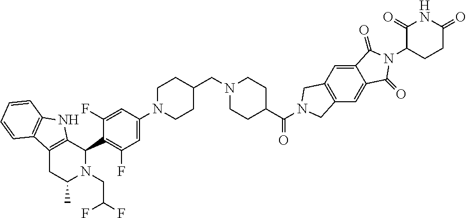 [SMILES: C[C@@H]1Cc2c([nH]c3ccccc23)[C@@H](c2c(F)cc(N3CCC(CN4CCC(C(=O)N5Cc6cc7c(cc6C5)C(=O)N(C5CCC(=O)NC5=O)C7=O)CC4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)piperidine-4-carbonyl)-2-(2,6- dioxopiperidin-3-yl)-6,7- dihydropyrrolo[3,4-f]isoindole- 1,3(2H,5H)-dione |
| E 239 | 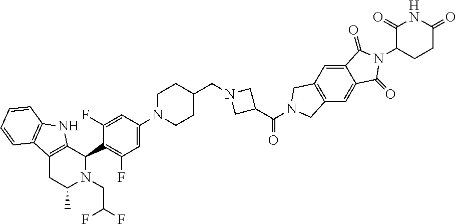 [SMILES: C[C@@H]1Cc2c([nH]c3ccccc23)[C@@H](c2c(F)cc(N3CCC(CN4CC(C(=O)N5Cc6cc7c(cc6C5)C(=O)N(C5CCC(=O)NC5=O)C7=O)C4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)azetidine-3-carbonyl)-2-(2,6- dioxopiperidin-3-yl)-6,7- dihydropyrrolo[3,4-f]isoindole- 1,3(2H,5H)-dione |
| E 240 | 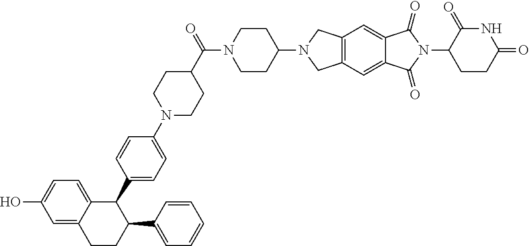 [SMILES: O=C1CCC(N2C(=O)c3cc4c(cc3C2=O)CN(C2CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-(1-(4- ((1R,2S)-6-hydroxy-2-phenyl-1,2,3,4- tetrahydronaphthalen-1- yl)phenyl)piperidine-4- carbonyl)piperidin-4-yl)-6,7- dihydropyrrolo[3,4-f]isoindole- 1,3(2H,5H)-dione |
| E 241 | 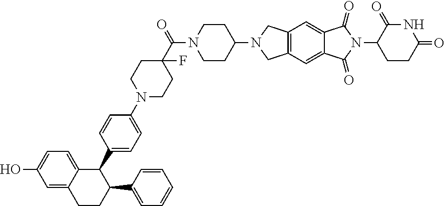 [SMILES: O=C1CCC(N2C(=O)c3cc4c(cc3C2=O)CN(C2CCN(C(=O)C3(F)CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-(4- fluoro-1-(4-((1R,2S)-6-hydroxy-2- phenyl-1,2,3,4-tetrahydronaphthalen-1- yl)phenyl)piperidine-4- carbonyl)piperidin-4-yl)-6,7- dihydropyrrolo[3,4-f]isoindole- 1,3(2H,5H)-dione |
| E 242 | 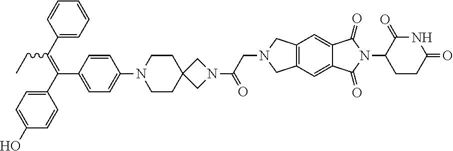 [SMILES: CCC(=C(c1ccc(O)cc1)c1ccc(N2CCC3(CC2)CN(C(=O)CN2Cc4cc5c(cc4C2)C(=O)N(C2CCC(=O)NC2=O)C5=O)C3)cc1)c1ccccc1] | 2-(2,6-dioxopiperidin-3-yl)-6-(2-(7-(4- (1-(4-hydroxyphenyl)-2-phenylbut-1-en- 1-yl)phenyl)-2,7-diazaspiro[3.5]nonan- 2-yl)-2-oxoethyl)-6,7- dihydropyrrolo[3,4-f]isoindole- 1,3(2H,5H)-dione |
| E243 | 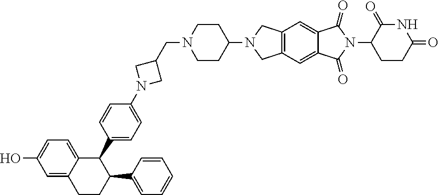 [SMILES: O=C1CCC(N2C(=O)c3cc4c(cc3C2=O)CN(C2CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)azetidin-3- yl)methyl)piperidin-4-yl)-6,7- dihydropyrrolo[3,4-f]isoindole- 1,3(2H,5H)-dione |
| E244 | 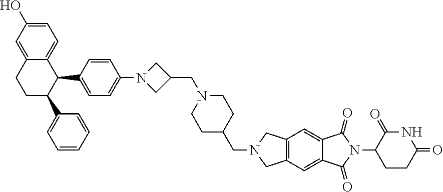 [SMILES: O=C1CCC(N2C(=O)c3cc4c(cc3C2=O)CN(CC2CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-((1-(4- ((1R,2S)-6-hydroxy-2-phenyl-1,2,3,4- tetrahydronaphthalen-1- yl)phenyl)azetidin-3- yl)methyl)piperidin-4-yl)methyl)-6,7- dihydropyrrolo[3,4-f]isoindole- 1,3(2H,5H)-dione |
| E245 | 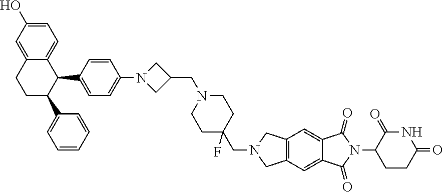 [SMILES: O=C1CCC(N2C(=O)c3cc4c(cc3C2=O)CN(CC2(F)CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-fluoro- 1-((1-(4-((1R,2S)-6-hydroxy-2-phenyl- 1,2,3,4-tetrahydronaphthalen-1- yl)phenyl)azetidin-3- yl)methyl)piperidin-4-yl)methyl)-6,7- dihydropyrrolo[3,4-f]isoindole- 1,3(2H,5H)-dione |
| E246 | 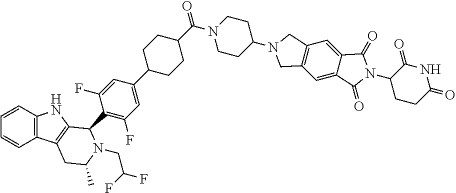 [SMILES: C[C@@H]1Cc2c([nH]c3ccccc23)[C@@H](c2c(F)cc(C3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4-(4-((1R,3R)-2-(2,2- difluoroethyl)-3-methyl-2,3,4,9- tetrahydro-1H-pyrido[3,4-b]indol-1-yl)- 3,5-difluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E247 | 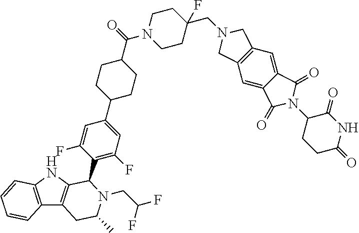 [SMILES: C[C@@H]1Cc2c([nH]c3ccccc23)[C@@H](c2c(F)cc(C3CCC(C(=O)N4CCC(F)(CN5Cc6cc7c(cc6C5)C(=O)N(C5CCC(=O)NC5=O)C7=O)CC4)CC3)cc2F)N1CC(F)F] | 6-((1-(4-(4-((1R,3R)-2-(2,2- difluoroethyl)-3-methyl-2,3,4,9- tetrahydro-1H-pyrido[3,4-b]indol-1-yl)- 3,5-difluorophenyl)cyclohexane-1- carbonyl)-4-fluoropiperidin-4- yl)methyl)-2-(2,6-dioxopiperidin-3-yl)- 6,7-dihydropyrrolo[3,4-f]isoindole- 1,3(2H,5H)-dione |
| E248 | 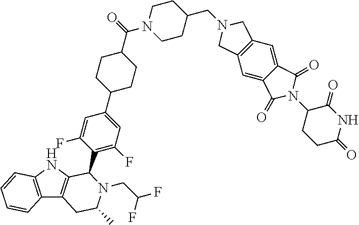 [SMILES: C[C@@H]1Cc2c([nH]c3ccccc23)[C@@H](c2c(F)cc(C3CCC(C(=O)N4CCC(CN5Cc6cc7c(cc6C5)C(=O)N(C5CCC(=O)NC5=O)C7=O)CC4)CC3)cc2F)N1CC(F)F] | 6-((1-(4-(4-((1R,3R)-2-(2,2- difluoroethyl)-3-methyl-2,3,4,9- tetrahydro-1H-pyrido[3,4-b]indol-1-yl)- 3,5-difluorophenyl)cyclohexane-1- carbonyl)piperidin-4-yl)methyl)-2-(2,6- dioxopiperidin-3-yl)-6,7- dihydropyrrolo[3,4-f]isoindole- 1,3(2H,5H)-dione |
| E249 | 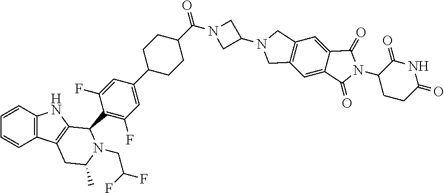 [SMILES: C[C@@H]1Cc2c([nH]c3ccccc23)[C@@H](c2c(F)cc(C3CCC(C(=O)N4CC(N5Cc6cc7c(cc6C5)C(=O)N(C5CCC(=O)NC5=O)C7=O)C4)CC3)cc2F)N1CC(F)F] | 6-(1-(4-(4-((1R,3R)-2-(2,2- difluoroethyl)-3-methyl-2,3,4,9- tetrahydro-1H-pyrido[3,4-b]indol-1-yl)- 3,5-difluorophenyl)cyclohexane-1- carbonyl)azetidin-3-yl)-2-(2,6- dioxopiperidin-3-yl)-6,7- dihydropyrrolo[3,4-f]isoindole- 1,3(2H,5H)-dione |
| E250 | 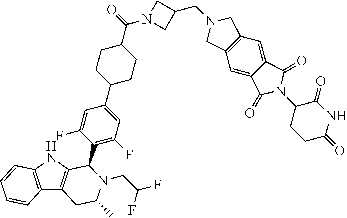 [SMILES: C[C@@H]1Cc2c([nH]c3ccccc23)[C@@H](c2c(F)cc(C3CCC(C(=O)N4CC(CN5Cc6cc7c(cc6C5)C(=O)N(C5CCC(=O)NC5=O)C7=O)C4)CC3)cc2F)N1CC(F)F] | 6-((1-(4-(4-((1R,3R)-2-(2,2- difluoroethyl)-3-methyl-2,3,4,9- tetrahydro-1H-pyrido[3,4-b]indol-1-yl)- 3,5-difluorophenyl)cyclohexane-1- carbonyl)azetidin-3-yl)methyl)-2-(2,6- dioxopiperidin-3-yl)-6,7- dihydropyrrolo[3,4-f]isoindole- 1,3(2H,5H)-dione |
| E 251 | 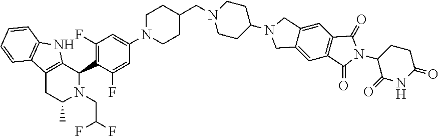 [SMILES: C[C@@H]1Cc2c([nH]c3ccccc23)[C@@H](c2c(F)cc(N3CCC(CN4CCC(N5Cc6cc7c(cc6C5)C(=O)N(C5CCC(=O)NC5=O)C7=O)CC4)CC3)cc2F)N1CC(F)F] | 6-(1-((1-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 252 | 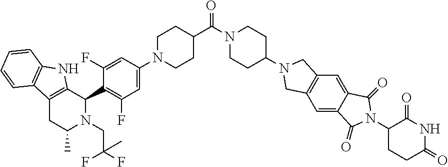 [SMILES: C[C@@H]1Cc2c([nH]c3ccccc23)[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(C)(F)F] | 6-(1-(1-(4-((1R,3R)-2-(2,2- difluoropropyl)-3-methyl-2,3,4,9- tetrahydro-1H-pyrido[3,4-b]indol-1-yl)- 3,5-difluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 253 | 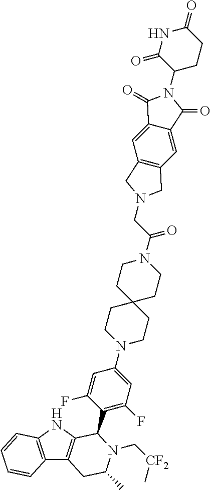 [SMILES: C[C@@H]1Cc2c([nH]c3ccccc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(C)(F)F] | 6-(2-(9-(4-((1R,3R)-2-(2,2- difluoropropyl)-3-methyl-2,3,4,9- tetrahydro-1H-pyrido[3,4-b]indol-1-yl)- 3,5-difluorophenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 254 | 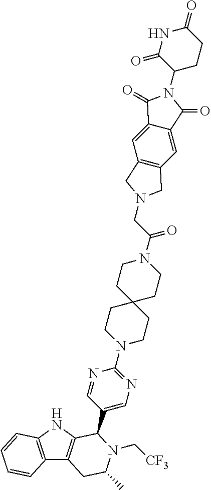 [SMILES: C[C@@H]1Cc2c([nH]c3ccccc23)[C@@H](c2cnc(N3CCC4(CCN(C(=O)CN5Cc6cc7c(cc6C5)C(=O)N(C5CCC(=O)NC5=O)C7=O)CC4)CC3)nc2)N1CC(F)(F)F] | 2-(2,6-dioxopiperidin-3-yl)-6-(2-(9-(5- ((1R,3R)-3-methyl-2-(2,2,2- trifluoroethyl)-2,3,4,9-tetrahydro-1H- pyrido[3,4-b]indol-1-yl)pyrimidin-2-yl)- 3,9-diazaspiro[5.5]undecan-3-yl)-2- oxoethyl)-6,7-dihydropyrrolo[3,4- f]isoindole-1,3(2H,5H)-dione |
| E 255 | 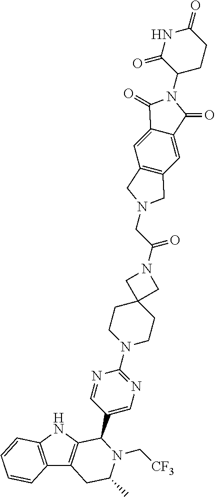 [SMILES: C[C@@H]1Cc2c([nH]c3ccccc23)[C@@H](c2cnc(N3CCC4(CC3)CN(C(=O)CN3Cc5cc6c(cc5C3)C(=O)N(C3CCC(=O)NC3=O)C6=O)C4)nc2)N1CC(F)(F)F] | 2-(2,6-dioxopiperidin-3-yl)-6-(2-(7-(5- ((1R,3R)-3-methyl-2-(2,2,2- trifluoroethyl)-2,3,4,9-tetrahydro-1H- pyrido[3,4-b]indol-1-yl)pyrimidin-2-yl)- 2,7-diazaspiro[3.5]nonan-2-yl)-2- oxoethyl)-6,7-dihydropyrrolo[3,4- f]isoindole-1,3(2H,5H)-dione |
| E 256 | 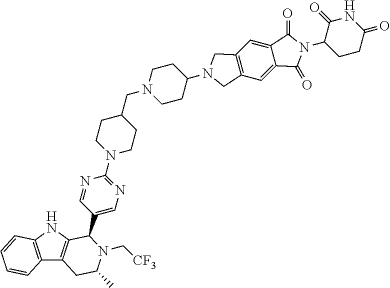 [SMILES: C[C@@H]1Cc2c([nH]c3ccccc23)[C@@H](c2cnc(N3CCC(CN4CCC(N5Cc6cc7c(cc6C5)C(=O)N(C5CCC(=O)NC5=O)C7=O)CC4)CC3)nc2)N1CC(F)(F)F] | 2-(2,6-dioxopiperidin-3-yl)-6-(1-((1-(5- ((1R,3R)-3-methyl-2-(2,2,2- trifluoroethyl)-2,3,4,9-tetrahydro-1H- pyrido[3,4-b]indol-1-yl)pyrimidin-2- yl)piperidin-4-yl)methyl)piperidin-4-yl)- 6,7-dihydropyrrolo[3,4-f]isoindole- 1,3(2H,5H)-dione |
| E 257 | 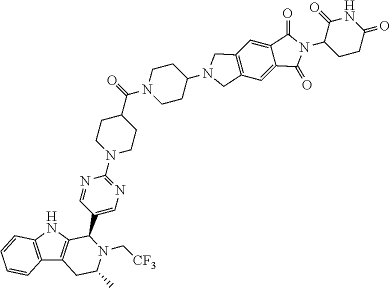 [SMILES: C[C@@H]1Cc2c([nH]c3ccccc23)[C@@H](c2cnc(N3CCC(C(=O)N4CCC(N5Cc6cc7c(cc6C5)C(=O)N(C5CCC(=O)NC5=O)C7=O)CC4)CC3)nc2)N1CC(F)(F)F] | 2-(2,6-dioxopiperidin-3-yl)-6-(1-(1-(5- ((1R,3R)-3-methyl-2-(2,2,2- trifluoroethyl)-2,3,4,9-tetrahydro-1H- pyrido[3,4-b]indol-1-yl)pyrimidin-2- yl)piperidine-4-carbonyl)piperidin-4-yl)- 6,7-dihydropyrrolo[3,4-f]isoindole- 1,3(2H,5H)-dione |
| E 258 | 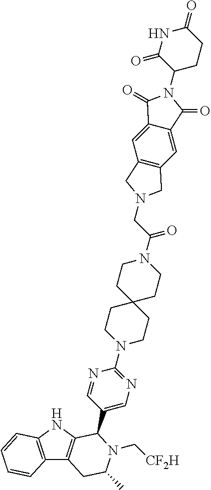 [SMILES: C[C@@H]1Cc2c([nH]c3ccccc23)[C@@H](c2cnc(N3CCC4(CCN(C(=O)CN5Cc6cc7c(cc6C5)C(=O)N(C5CCC(=O)NC5=O)C7=O)CC4)CC3)nc2)N1CC(F)F] | 6-(2-(9-(5-((1R,3R)-2-(2,2- difluoroethyl)-3-methyl-2,3,4,9- tetrahydro-1H-pyrido[3,4-b ]indol-1- yl)pyrimidin-2-yl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 259 | 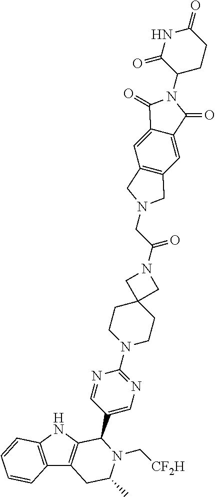 [SMILES: C[C@@H]1Cc2c([nH]c3ccccc23)[C@@H](c2cnc(N3CCC4(CC3)CN(C(=O)CN3Cc5cc6c(cc5C3)C(=O)N(C3CCC(=O)NC3=O)C6=O)C4)nc2)N1CC(F)F] | 6-(2-(7-(5-((1R,3R)-2-(2,2- difluoroethyl)-3-methyl-2,3,4,9- tetrahydro-1H-pyrido[3,4-b]indol-1- yl)pyrimidin-2-yl)-2,7- diazaspiro[3.5]nonan-2-yl)-2-oxoethyl)- 2-(2,6-dioxopiperidin-3-yl)-6,7- dihydropyrrolo[3,4-f]isoindole- 1,3(2H,5H)-dione |
| E 260 | 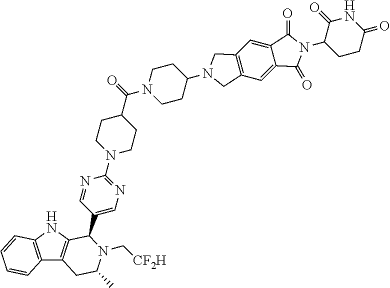 [SMILES: C[C@@H]1Cc2c([nH]c3ccccc23)[C@@H](c2cnc(N3CCC(C(=O)N4CCC(N5Cc6cc7c(cc6C5)C(=O)N(C5CCC(=O)NC5=O)C7=O)CC4)CC3)nc2)N1CC(F)F] | 6-(1-(1-(5-((1R,3R)-2-(2,2- difluoroethyl)-3-methyl-2,3,4,9- tetrahydro-1H-pyrido[3,4-b]indol-1- yl)pyrimidin-2-yl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 261 | 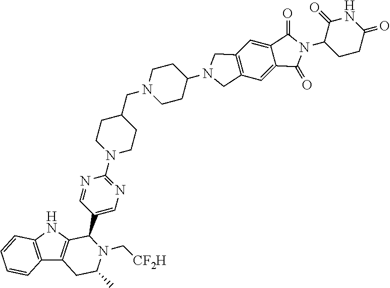 [SMILES: C[C@@H]1Cc2c([nH]c3ccccc23)[C@@H](c2cnc(N3CCC(CN4CCC(N5Cc6cc7c(cc6C5)C(=O)N(C5CCC(=O)NC5=O)C7=O)CC4)CC3)nc2)N1CC(F)F] | 6-(1-((1-(5-((1R,3R)-2-(2,2- difluoroethyl)-3-methyl-2,3,4,9- tetrahydro-1H-pyrido[3,4-b]indol-1- yl)pyrimidin-2-yl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 262 | 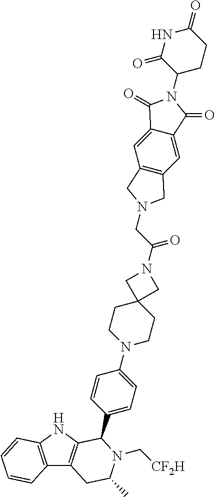 [SMILES: C[C@@H]1Cc2c([nH]c3ccccc23)[C@@H](c2ccc(N3CCC4(CC3)CN(C(=O)CN3Cc5cc6c(cc5C3)C(=O)N(C3CCC(=O)NC3=O)C6=O)C4)cc2)N1CC(F)F] | 6-(2-(7-(4-((1R,3R)-2-(2,2- difluoroethyl)-3-methyl-2,3,4,9- tetrahydro-1H-pyrido[3,4-b]indol-1- yl)phenyl)-2,7-diazaspiro[3.5]nonan-2- yl)-2-oxoethyl)-2-(2,6-dioxopiperidin-3- yl)-6,7-dihydropyrrolo[3,4-f]isoindole- 1,3(2H,5H)-dione |
| E 263 | 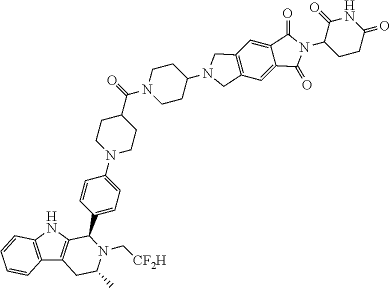 [SMILES: C[C@@H]1Cc2c([nH]c3ccccc23)[C@@H](c2ccc(N3CCC(C(=O)N4CCC(N5Cc6cc7c(cc6C5)C(=O)N(C5CCC(=O)NC5=O)C7=O)CC4)CC3)cc2)N1CC(F)F] | 6-(1-(1-(4-((1R,3R)-2-(2,2- difluoroethyl)-3-methyl-2,3,4,9- tetrahydro-1H-pyrido[3,4-b]indol-1- yl)phenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 264 | 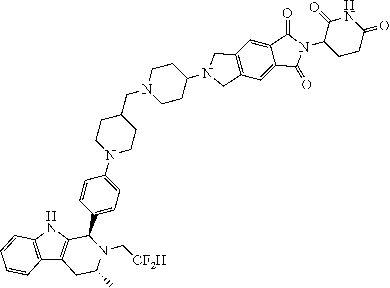 [SMILES: C[C@@H]1Cc2c([nH]c3ccccc23)[C@@H](c2ccc(N3CCC(CN4CCC(N5Cc6cc7c(cc6C5)C(=O)N(C5CCC(=O)NC5=O)C7=O)CC4)CC3)cc2)N1CC(F)F] | 6-(1-((1-(4-((1R,3R)-2-(2,2- difluoroethyl)-3-methyl-2,3,4,9- tetrahydro-1H-pyrido[3,4-b]indol-1- yl)phenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 265 | 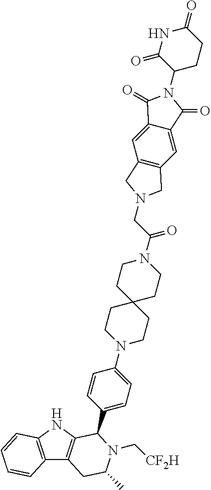 [SMILES: C[C@@H]1Cc2c([nH]c3ccccc23)[C@@H](c2ccc(N3CCC4(CCN(C(=O)CN5Cc6cc7c(cc6C5)C(=O)N(C5CCC(=O)NC5=O)C7=O)CC4)CC3)cc2)N1CC(F)F] | 6-(2-(9-(4-((1R,3R)-2-(2,2- difluoroethyl)-3-methyl-2,3,4,9- tetrahydro-1H-pyrido[3,4-b]indol-1- yl)phenyl)-3,9-diazaspiro[5.5]undecan- 3-yl)-2-oxoethyl)-2-(2,6-dioxopiperidin- 3-yl)-6,7-dihydropyrrolo[3,4- f]isoindole-1,3(2H,5H)-dione |
| E266 | 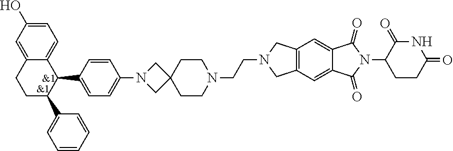 [SMILES: O=C1CCC(N2C(=O)c3cc4c(cc3C2=O)CN(CCN2CCC3(CC2)CN(c2ccc([C@@H]5c6ccc(O)cc6CC[C@@H]5c5ccccc5)cc2)C3)C4)C(=O)N1] | rac-2-(2,6-dioxopiperidin-3-yl)-6-(2-(2- (4-((1R,2S)-6-hydroxy-2-phenyl- 1,2,3,4-tetrahydronaphthalen-1- yl)phenyl)-2,7-diazaspiro[3.5]nonan-7- yl)ethyl)-6,7-dihydropyrrolo[3,4- f]isoindole-1,3(2H,5H)-dione |
| E267 | 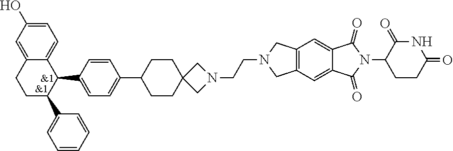 [SMILES: O=C1CCC(N2C(=O)c3cc4c(cc3C2=O)CN(CCN2CC3(CCC(c5ccc([C@@H]6c7ccc(O)cc7CC[C@@H]6c6ccccc6)cc5)CC3)C2)C4)C(=O)N1] | rac-2-(2,6-dioxopiperidin-3-yl)-6-(2-(7- (4-((1R,2S)-6-hydroxy-2-phenyl- 1,2,3,4-tetrahydronaphthalen-1- yl)phenyl)-2,7-diazaspiro[3.5]nonan-2- yl)ethyl)-6,7-dihydropyrrolo[3,4- f]isoindole-1,3(2H,5H)-dione |
| E268 | 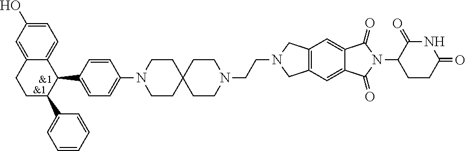 [SMILES: O=C1CCC(N2C(=O)c3cc4c(cc3C2=O)CN(CCN2CCC3(CC2)CCN(c2ccc([C@@H]5c6ccc(O)cc6CC[C@@H]5c5ccccc5)cc2)CC3)C4)C(=O)N1] | rac-2-(2,6-dioxopiperidin-3-yl)-6-(2-(9- (4-((1R,2S)-6-hydroxy-2-phenyl- 1,2,3,4-tetrahydronaphthalen-1- yl)phenyl)-3,9-diazaspiro[5.5]undecan- 3-yl)ethyl)-6,7-dihydropyrrolo[3,4- f]isoindole-1,3(2H,5H)-dione |
| E 269 | 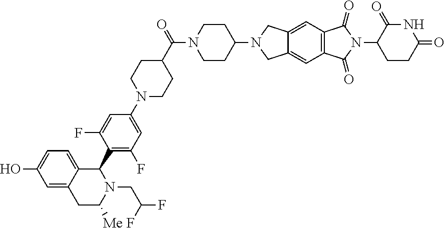 [SMILES: C[C@@H]1Cc2cc(O)ccc2[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((1S,3R)-2-(2,2- difluoroethyl)-6-hydroxy-3-methyl- 1,2,3,4-tetrahydroisoquinolin-1-yl)-3,5- difluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 270 | 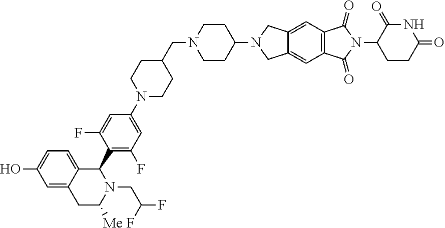 [SMILES: C[C@@H]1Cc2cc(O)ccc2[C@@H](c2c(F)cc(N3CCC(CN4CCC(N5Cc6cc7c(cc6C5)C(=O)N(C5CCC(=O)NC5=O)C7=O)CC4)CC3)cc2F)N1CC(F)F] | 6-(1-((1-(4-((1S,3R)-2-(2,2- difluoroethyl)-6-hydroxy-3-methyl- 1,2,3,4-tetrahydroisoquinolin-1-yl)-3,5- difluorophenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 271 | 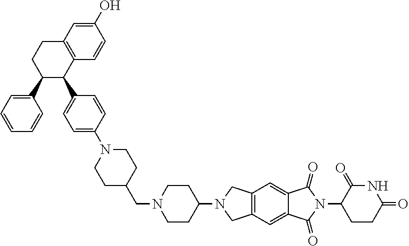 [SMILES: O=C1CCC(N2C(=O)c3cc4c(cc3C2=O)CN(C2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)methyl)piperidin-4-yl)-6,7- dihydropyrrolo[3,4-f]isoindole- 1,3(2H,5H)-dione |
| E 272 | 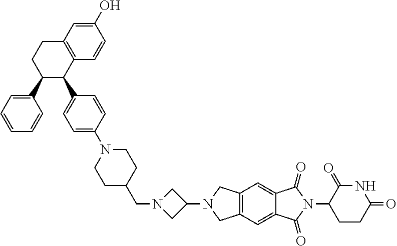 [SMILES: O=C1CCC(N2C(=O)c3cc4c(cc3C2=O)CN(C2CN(CC3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)methyl)azetidin-3-yl)-6,7- dihydropyrrolo[3,4-f]isoindole- 1,3(2H,5H)-dione |
| E 273 | 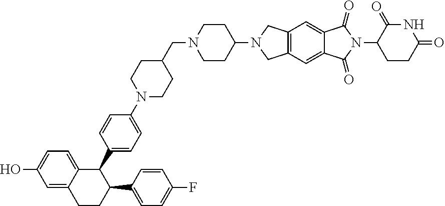 [SMILES: O=C1CCC(N2C(=O)c3cc4c(cc3C2=O)CN(C2CCN(CC3CCN(c5ccc([C@@H]6c7ccc(O)cc7CC[C@@H]6c6ccc(F)cc6)cc5)CC3)CC2)C4)C(=O)N1] | 2-(2,6-dioxopiperidin-3-yl)-6-(1-((1-(4- ((1R,2S)-2-(4-fluorophenyl)-6-hydroxy- 1,2,3,4-tetrahydronaphthalen-1- yl)phenyl)piperidin-4- yl)methyl)piperidin-4-yl)-6,7- dihydropyrrolo[3,4-f]isoindole- 1,3(2H,5H)-dione |
| E 274 | 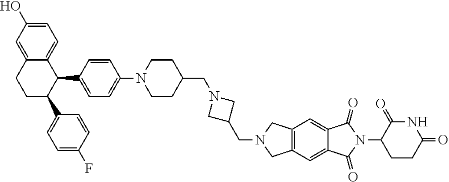 [SMILES: O=C1CCC(N2C(=O)c3cc4c(cc3C2=O)CN(CC2CN(CC3CCN(c5ccc([C@@H]6c7ccc(O)cc7CC[C@@H]6c6ccc(F)cc6)cc5)CC3)C2)C4)C(=O)N1] | 2-(2,6-dioxopiperidin-3-yl)-6-((1-((1-(4- ((1R,2S)-2-(4-fluorophenyl)-6-hydroxy- 1,2,3,4-tetrahydronaphthalen-1- yl)phenyl)piperidin-4- yl)methyl)azetidin-3-yl)methyl)-6,7- dihydropyrrolo[3,4-f]isoindole- 1,3(2H,5H)-dione |
| E 275 | 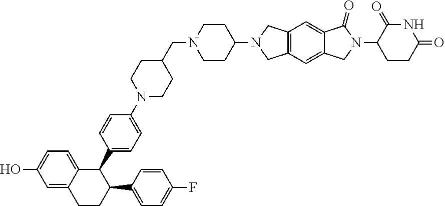 [SMILES: O=C1CCC(N2Cc3cc4c(cc3C2=O)CN(C2CCN(CC3CCN(c5ccc([C@@H]6c7ccc(O)cc7CC[C@@H]6c6ccc(F)cc6)cc5)CC3)CC2)C4)C(=O)N1] | 3-(6-(1-((1-(4-((1R,2S)-2-(4- fluorophenyl)-6-hydroxy-1,2,3,4- tetrahydronaphthalen-1- yl)phenyl)piperidin-4- yl)methyl)piperidin-4-yl)-1-oxo-3,5,6,7- tetrahydropyrrolo[3,4-f]isoindol-2(1H)- yl)piperidine-2,6-dione |
| E 276 | 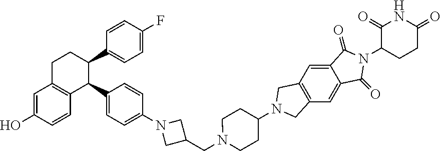 [SMILES: O=C1CCC(N2C(=O)c3cc4c(cc3C2=O)CN(C2CCN(CC3CN(c5ccc([C@H]6c7ccc(O)cc7CC[C@H]6c6ccc(F)cc6)cc5)C3)CC2)C4)C(=O)N1] | 2-(2,6-dioxopiperidin-3-yl)-6-(1-((1-(4- ((1S,2R)-2-(4-fluorophenyl)-6-hydroxy- 1,2,3,4-tetrahydronaphthalen-1- yl)phenyl)azetidin-3- yl)methyl)piperidin-4-yl)-6,7- dihydropyrrolo[3,4-f]isoindole- 1,3(2H,5H)-dione |
| E 277 | 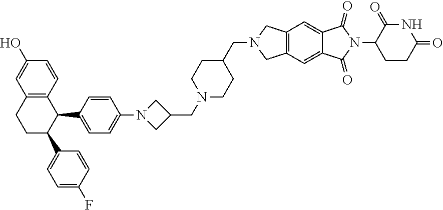 [SMILES: O=C1CCC(N2C(=O)c3cc4c(cc3C2=O)CN(CC2CCN(CC3CN(c5ccc([C@@H]6c7ccc(O)cc7CC[C@@H]6c6ccc(F)cc6)cc5)C3)CC2)C4)C(=O)N1] | 2-(2,6-dioxopiperidin-3-yl)-6-((1-((1-(4- ((1R,2S)-2-(4-fluorophenyl)-6-hydroxy- 1,2,3,4-tetrahydronaphthalen-1- yl)phenyl)azetidin-3- yl)methyl)piperidin-4-yl)methyl)-6,7- dihydropyrrolo[3,4-f]isoindole- 1,3(2H,5H)-dione |
| E 278 | 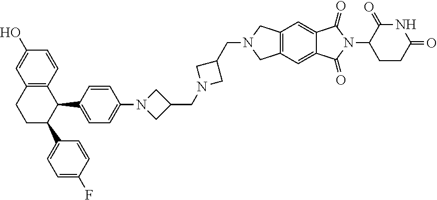 [SMILES: O=C1CCC(N2C(=O)c3cc4c(cc3C2=O)CN(CC2CN(CC3CN(c5ccc([C@@H]6c7ccc(O)cc7CC[C@@H]6c6ccc(F)cc6)cc5)C3)C2)C4)C(=O)N1] | 2-(2,6-dioxopiperidin-3-yl)-6-((1-((1-(4- ((1R,2S)-2-(4-fluorophenyl)-6-hydroxy- 1,2,3,4-tetrahydronaphthalen-1- yl)phenyl)azetidin-3-yl)methyl)azetidin- 3-yl)methyl)-6,7-dihydropyrrolo[3,4- f]isoindole-1,3(2H,5H)-dione |
| E 279 | 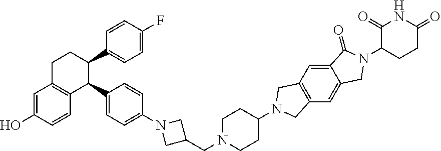 [SMILES: O=C1CCC(N2Cc3cc4c(cc3C2=O)CN(C2CCN(CC3CN(c5ccc([C@H]6c7ccc(O)cc7CC[C@H]6c6ccc(F)cc6)cc5)C3)CC2)C4)C(=O)N1] | 3-(6-(1-((1-(4-((1S,2R)-2-(4- fluorophenyl)-6-hydroxy-1,2,3,4- tetrahydronaphthalen-1- yl)phenyl)azetidin-3- yl)methyl)piperidin-4-yl)-1-oxo-3,5,6,7- tetrahydropyrrolo[3,4-f]isoindol-2(1H)- yl)piperidine-2,6-dione |
| E 280 | 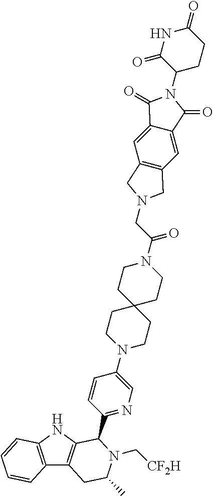 [SMILES: C[C@@H]1Cc2c([nH]c3ccccc23)[C@@H](c2ccc(N3CCC4(CCN(C(=O)CN5Cc6cc7c(cc6C5)C(=O)N(C5CCC(=O)NC5=O)C7=O)CC4)CC3)cn2)N1CC(F)F] | 6-(2-(9-(6-((1S,3R)-2-(2,2- difluoroethyl)-3-methyl-2,3,4,9- tetrahydro-1H-pyrido[3,4-b]indol-1- yl)pyridin-3-yl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 281 | 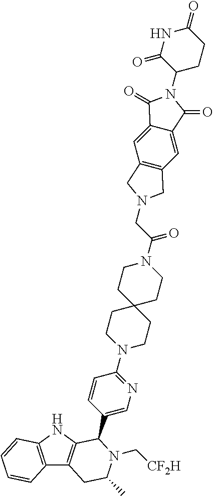 [SMILES: C[C@@H]1Cc2c([nH]c3ccccc23)[C@@H](c2ccc(N3CCC4(CCN(C(=O)CN5Cc6cc7c(cc6C5)C(=O)N(C5CCC(=O)NC5=O)C7=O)CC4)CC3)nc2)N1CC(F)F] | 6-(2-(9-(5-((1R,3R)-2-(2,2- difluoroethyl)-3-methyl-2,3,4,9- tetrahydro-1H-pyrido[3,4-b]indol-1- yl)pyridin-2-yl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 282 | 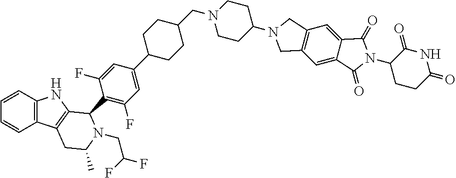 [SMILES: C[C@@H]1Cc2c([nH]c3ccccc23)[C@@H](c2c(F)cc(C3CCC(CN4CCC(N5Cc6cc7c(cc6C5)C(=O)N(C5CCC(=O)NC5=O)C7=O)CC4)CC3)cc2F)N1CC(F)F] | 6-(1-((4-(4-((1R,3R)-2-(2,2- difluoroethyl)-3-methyl-2,3,4,9- tetrahydro-1H-pyrido[3,4-b]indol-1-yl)- 3,5- difluorophenyl)cyclohexyl)methyl)piper idin-4-yl)-2-(2,6-dioxopiperidin-3-yl)- 6,7-dihydropyrrolo[3,4-f]isoindole- 1,3(2H,5H)-dione |
| E 283 | 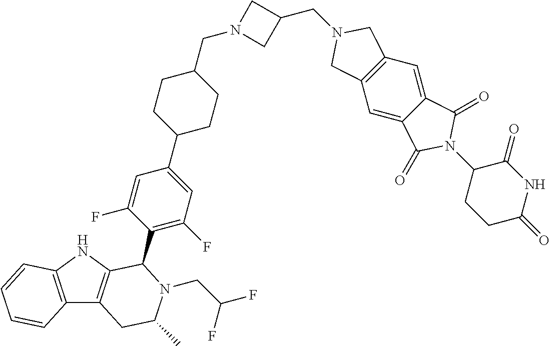 [SMILES: C[C@@H]1Cc2c([nH]c3ccccc23)[C@@H](c2c(F)cc(C3CCC(CN4CC(CN5Cc6cc7c(cc6C5)C(=O)N(C5CCC(=O)NC5=O)C7=O)C4)CC3)cc2F)N1CC(F)F] | 6-((1-((4-(4-((1R,3R)-2-(2,2- difluoroethyl)-3-methyl-2,3,4,9- tetrahydro-1H-pyrido[3,4-b]indol-1-yl)- 3,5- difluorophenyl)cyclohexyl)methyl)azeti din-3-yl)methyl)-2-(2,6-dioxopiperidin- 3-yl)-6,7-dihydropyrrolo[3,4- f]isoindole-1,3(2H,5H)-dione |
| E284 | 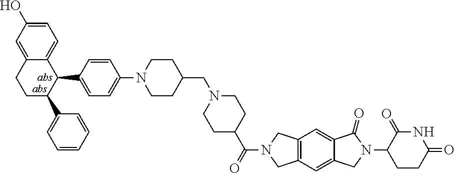 [SMILES: O=C1CCC(N2Cc3cc4c(cc3C2=O)CN(C(=O)C2CCN(CC3CCN(c5ccc([C@@H]6c7ccc(O)cc7CC[C@@H]6c6ccccc6)cc5)CC3)CC2)C4)C(=O)N1] | 3-(6-(1-((1-(4-((1R,2S)-6-hydroxy-2- phenyl-1,2,3,4-tetrahydronaphthalen-1- yl)phenyl)piperidin-4- yl)methyl)piperidine-4-carbonyl)-1-oxo- 3,5,6,7-tetrahydropyrrolo[3,4-f]isoindol- 2(1H)-yl)piperidine-2,6-dione |
| E 285 | 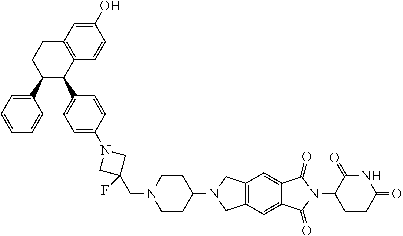 [SMILES: O=C1CCC(N2C(=O)c3cc4c(cc3C2=O)CN(C2CCN(CC3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((3- fluoro-1-(4-((1R,2S)-6-hydroxy-2- phenyl-1,2,3,4-tetrahydronaphthalen-1- yl)phenyl)azetidin-3- yl)methyl)piperidin-4-yl)-6,7- dihydropyrrolo[3,4-f]isoindole- 1,3(2H,5H)-dione |
| E 286 | 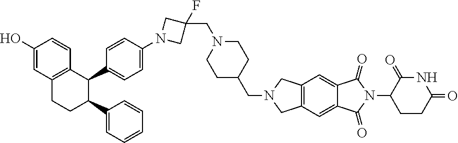 [SMILES: O=C1CCC(N2C(=O)c3cc4c(cc3C2=O)CN(CC2CCN(CC3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-((3- fluoro-1-(4-((1R,2S)-6-hydroxy-2- phenyl-1,2,3,4-tetrahydronaphthalen-1- yl)phenyl)azetidin-3- yl)methyl)piperidin-4-yl)methyl)-6,7- dihydropyrrolo[3,4-f]isoindole- 1,3(2H,5H)-dione |
| E287 | 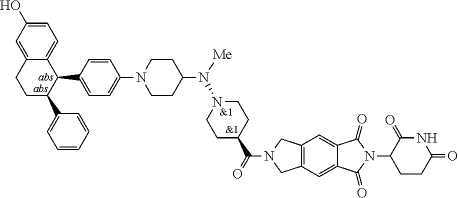 | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)(methyl)amino)piperidine-4- carbonyl)-6,7-dihydropyrrolo[3,4- f]isoindole-1,3(2H,5H)-dione |
| E288 | 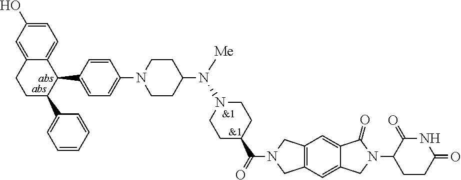 | 3-(6-(1-((1-(4-((1R,2S)-6-hydroxy-2- phenyl-1,2,3,4-tetrahydronaphthalen-1- yl)phenyl)piperidin-4- yl)(methyl)amino)piperidine-4- carbonyl)-1-oxo-3,5,6,7- tetrahydropyrrolo[3,4-f]isoindol-2(1H)- yl)piperidine-2,6-dione |
| E 289 | 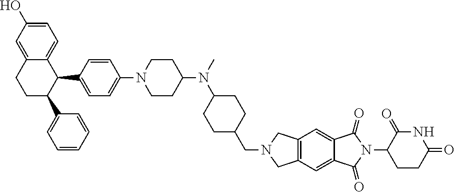 [SMILES: CN(C1CCC(CN2Cc3cc4c(cc3C2)C(=O)N(C2CCC(=O)NC2=O)C4=O)CC1)C1CCN(c2ccc([C@@H]3c4ccc(O)cc4CC[C@@H]3c3ccccc3)cc2)CC1] | 2-(2,6-dioxopiperidin-3-yl)-6-((4-((1-(4- ((1R,2S)-6-hydroxy-2-phenyl-1,2,3,4- tetrahydronaphthalen-1- yl)phenyl)piperidin-4- yl)(methyl)amino)cyclohexyl)methyl)- 6,7-dihydropyrrolo[3,4-f]isoindole- 1,3(2H,5H)-dione |
| E 290 | 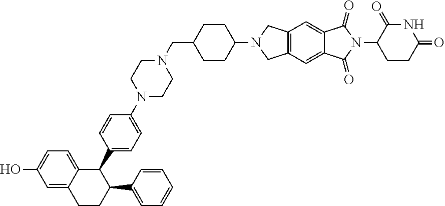 [SMILES: O=C1CCC(N2C(=O)c3cc4c(cc3C2=O)CN(C2CCC(CN3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-((4-(4- ((1R,2S)-6-hydroxy-2-phenyl-1,2,3,4- tetrahydronaphthalen-1- yl)phenyl)piperazin-1- yl)methyl)cyclohexyl)-6,7- dihydropyrrolo[3,4-f]isoindole- 1,3(2H,5H)-dione |
| E 291 | 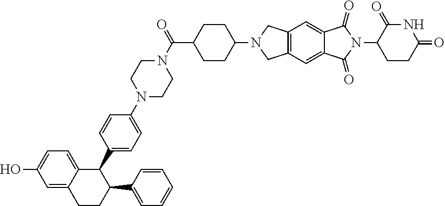 [SMILES: O=C1CCC(N2C(=O)c3cc4c(cc3C2=O)CN(C2CCC(C(=O)N3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-(4-(4- ((1R,2S)-6-hydroxy-2-phenyl-1,2,3,4- tetrahydronaphthalen-1- yl)phenyl)piperazine-1- carbonyl)cyclohexyl)-6,7- dihydropyrrolo[3,4-f]isoindole- 1,3(2H,5H)-dione |
| E 292 | 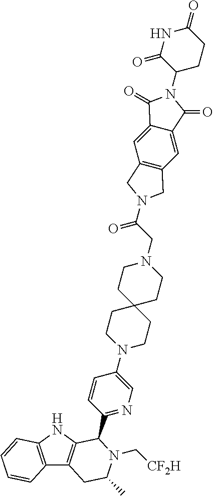 [SMILES: C[C@@H]1Cc2c([nH]c3ccccc23)[C@@H](c2ccc(N3CCC4(CCN(CC(=O)N5Cc6cc7c(cc6C5)C(=O)N(C5CCC(=O)NC5=O)C7=O)CC4)CC3)cn2)N1CC(F)F] | 6-(2-(9-(6-((1S,3R)-2-(2,2- difluoroethyl)-3-methyl-2,3,4,9- tetrahydro-1H-pyrido[3,4-b ]indol-1- yl)pyridin-3-yl)-3,9- diazaspiro[5.5]undecan-3-yl)acetyl)-2- (2,6-dioxopiperidin-3-yl)-6,7- dihydropyrrolo[3,4-f]isoindole- 1,3(2H,5H)-dione |
| E 293 | 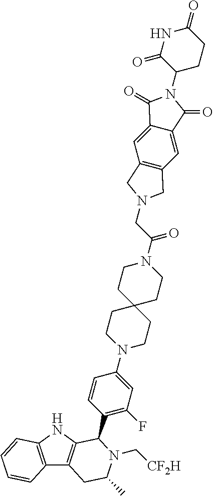 [SMILES: C[C@@H]1Cc2c([nH]c3ccccc23)[C@@H](c2ccc(N3CCC4(CCN(C(=O)CN5Cc6cc7c(cc6C5)C(=O)N(C5CCC(=O)NC5=O)C7=O)CC4)CC3)cc2F)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3-fluorophenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 294 | 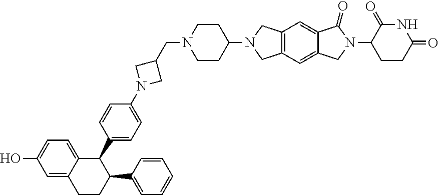 [SMILES: O=C1CCC(N2Cc3cc4c(cc3C2=O)CN(C2CCN(CC3CN(c5ccc([C@@H]6c7ccc(O)cc7CC[C@@H]6c6ccccc6)cc5)C3)CC2)C4)C(=O)N1] | 3-(6-(1-((1-(4-((1R,2S)-6-hydroxy-2- phenyl-1,2,3,4-tetrahydronaphthalen-1- yl)phenyl)azetidin-3- yl)methyl)piperidin-4-yl)-1-oxo-3,5,6,7- tetrahydropyrrolo[3,4-f]isoindol-2(1H)- yl)piperidine-2,6-dione |
| E 295 | 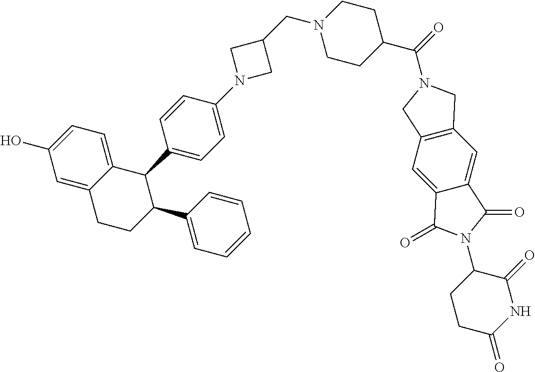 [SMILES: O=C1CCC(N2C(=O)c3cc4c(cc3C2=O)CN(C(=O)C2CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)azetidin-3- yl)methyl)piperidine-4-carbonyl)-6,7- dihydropyrrolo[3,4-f]isoindole- 1,3(2H,5H)-dione |
| E 296 | 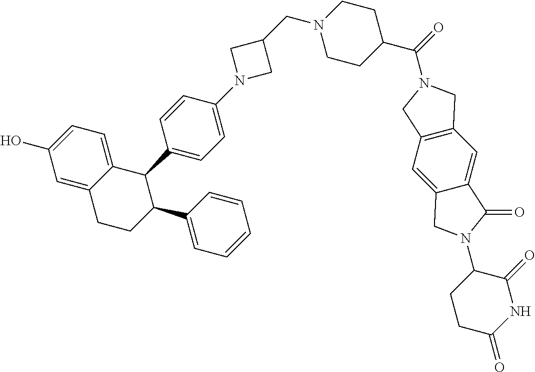 [SMILES: O=C1CCC(N2Cc3cc4c(cc3C2=O)CN(C(=O)C2CCN(CC3CN(c5ccc([C@@H]6c7ccc(O)cc7CC[C@@H]6c6ccccc6)cc5)C3)CC2)C4)C(=O)N1] | 3-(6-(1-((1-(4-((1R,2S)-6-hydroxy-2- phenyl-1,2,3,4-tetrahydronaphthalen-1- yl)phenyl)azetidin-3- yl)methyl)piperidine-4-carbonyl)-1-oxo- 3,5,6,7-tetrahydropyrrolo[3,4-f]isoindol- 2(1H)-yl)piperidine-2,6-dione |
| E 297 | 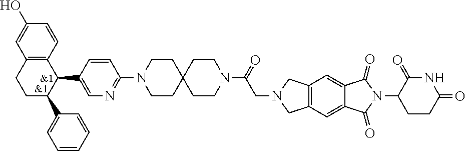 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cn2)CC3)C4)C(=O)N1] | Rac-2-(2,6-dioxopiperidin-3-yl)-6-(2-(9- (5-((1S,2S)-6-hydroxy-2-phenyl-1,2,3,4- tetrahydronaphthalen-1-yl)pyridin-2-yl)- 3,9-diazaspiro[5.5]undecan-3-yl)-2- oxoethyl)-6,7-dihydropyrrolo[3,4- f]isoindole-1,3(2H,5H)-dione |
| E 298 | 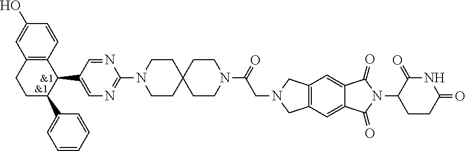 [SMILES: O=C1CCC(N2C(=O)c3cc4c(cc3C2=O)CN(CC(=O)N2CCC3(CC2)CCN(c2ncc([C@@H]5c6ccc(O)cc6CC[C@@H]5c5ccccc5)cn2)CC3)C4)C(=O)N1] | Rac-2-(2,6-dioxopiperidin-3-yl)-6-(2-(9- (5-((1S,2S)-6-hydroxy-2-phenyl-1,2,3,4- tetrahydronaphthalen-1-yl)pyrimidin-2- yl)-3,9-diazaspiro[5.5]undecan-3-yl)-2- oxoethyl)-6,7-dihydropyrrolo[3,4- f]isoindole-1,3(2H,5H)-dione |
| E 299 | 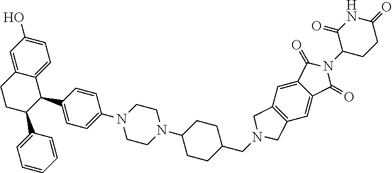 [SMILES: O=C1CCC(N2C(=O)c3cc4c(cc3C2=O)CN(CC2CCC(N3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((4-(4-(4- ((1R,2S)-6-hydroxy-2-phenyl-1,2,3,4- tetrahydronaphthalen-1- yl)phenyl)piperazin-1- yl)cyclohexyl)methyl)-6,7- dihydropyrrolo[3,4-f]isoindole- 1,3(2H,5H)-dione |
| E300 | 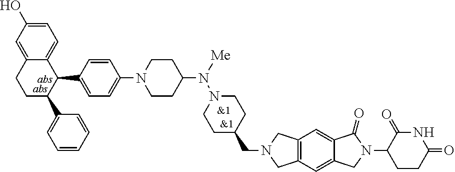 | 3-(6-((1-((1-(4-((1R,2S)-6-hydroxy-2- phenyl-1,2,3,4-tetrahydronaphthalen-1- yl)phenyl)piperidin-4- yl)(methyl)amino)piperidin-4- yl)methyl)-1-oxo-3,5,6,7- tetrahydropyrrolo[3,4-f]isoindol-2(1H)- yl)piperidine-2,6-dione |
| E301 | 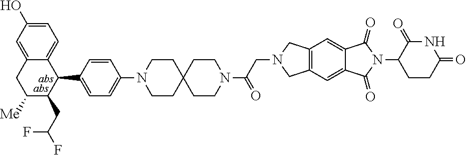 [SMILES: C[C@@H]1Cc2cc(O)ccc2[C@@H](c2ccc(N3CCC4(CCN(C(=O)CN5Cc6cc7c(cc6C5)C(=O)N(C5CCC(=O)NC5=O)C7=O)CC4)CC3)cc2)[C@H]1CC(F)F] | 6-(2-(9-(4-((1R,3R)-2-(2,2- difluoroethyl)-6-hydroxy-3-methyl- 1,2,3,4-tetrahydroisoquinolin-1- yl)phenyl)-3,9-diazaspiro[5.5]undecan- 3-yl)-2-oxoethyl)-2-(2,6-dioxopiperidin- 3-yl)-6,7-dihydropyrrolo[3,4- f]isoindole-1,3(2H,5H)-dione |
| E 302 | 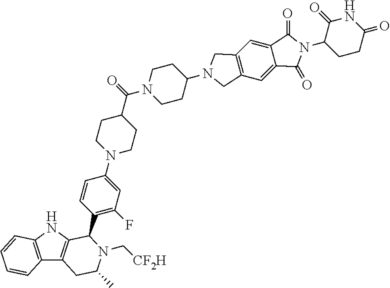 [SMILES: C[C@@H]1Cc2c([nH]c3ccccc23)[C@@H](c2ccc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((1R,3R)-2-(2,2- difluoroethyl)-3-methyl-2,3,4,9- tetrahydro-1H-pyrido[3,4-b]indol-1-yl)- 3-fluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 303 | 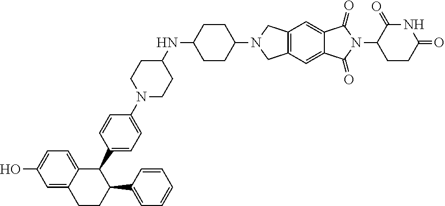 [SMILES: O=C1CCC(N2C(=O)c3cc4c(cc3C2=O)CN(C2CCC(NC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-((1-(4- ((1R,2S)-6-hydroxy-2-phenyl-1,2,3,4- tetrahydronaphthalen-1- yl)phenyl)piperidin-4- yl)amino)cyclohexyl)-6,7- dihydropyrrolo[3,4-f]isoindole- 1,3(2H,5H)-dione |
| E 304 | 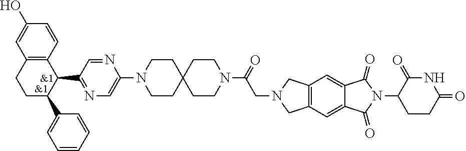 [SMILES: O=C1CCC(N2C(=O)c3cc4c(cc3C2=O)CN(CC(=O)N2CCC3(CC2)CCN(c2cnc([C@@H]5c6ccc(O)cc6CC[C@@H]5c5ccccc5)cn2)CC3)C4)C(=O)N1] | Rac-2-(2,6-dioxopiperidin-3-yl)-6-(2-(9- (5-((1S,2S)-6-hydroxy-2-phenyl-1,2,3,4- tetrahydronaphthalen-1-yl)pyrazin-2-yl)- 3,9-diazaspiro[5.5]undecan-3-yl)-2- oxoethyl)-6,7-dihydropyrrolo[3,4- f]isoindole-1,3(2H,5H)-dione |
| E 305 | 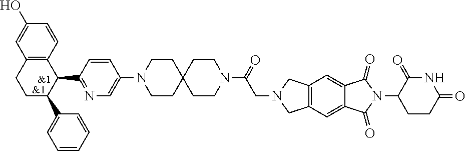 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)nc2)CC3)C4)C(=O)N1] | Rac-2-(2,6-dioxopiperidin-3-yl)-6-(2-(9- (6-((1S,2S)-6-hydroxy-2-phenyl-1,2,3,4- tetrahydronaphthalen-1-yl)pyridin-3-yl)- 3,9-diazaspiro[5.5]undecan-3-yl)-2- oxoethyl)-6,7-dihydropyrrolo[3,4- f]isoindole-1,3(2H,5H)-dione |
| E 306 | 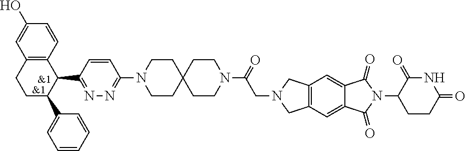 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)nn2)CC3)C4)C(=O)N1] | Rac-2-(2,6-dioxopiperidin-3-yl)-6-(2-(9- (6-((1S,2S)-6-hydroxy-2-phenyl-1,2,3,4- tetrahydronaphthalen-1-yl)pyridazin-3- yl)-3,9-diazaspiro[5.5]undecan-3-yl)-2- oxoethyl)-6,7-dihydropyrrolo[3,4- f]isoindole-1,3(2H,5H)-dione |
| E 307 | 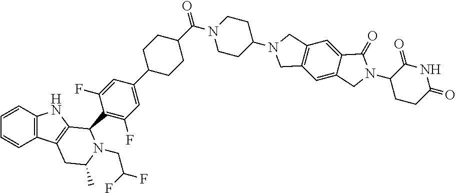 [SMILES: C[C@@H]1Cc2c([nH]c3ccccc23)[C@@H](c2c(F)cc(C3CCC(C(=O)N4CCC(N5Cc6cc7c(cc6C5)C(=O)N(C5CCC(=O)NC5=O)C7)CC4)CC3)cc2F)N1CC(F)F] | 3-(6-(1-(4-(4-((1R,3R)-2-(2,2- difluoroethyl)-3-methyl-2,3,4,9- tetrahydro-1H-pyrido[3,4-b]indol-1-yl)- 3,5-difluorophenyl)cyclohexane-1- carbonyl)piperidin-4-yl)-1-oxo-3,5,6,7- tetrahydropyrrolo[3,4-f]isoindol-2(1H)- yl)piperidine-2,6-dione |
| E 308 | 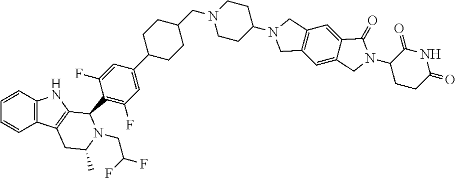 [SMILES: C[C@@H]1Cc2c([nH]c3ccccc23)[C@@H](c2c(F)cc(C3CCC(CN4CCC(N5Cc6cc7c(cc6C5)C(=O)N(C5CCC(=O)NC5=O)C7)CC4)CC3)cc2F)N1CC(F)F] | 3-(6-(1-((4-(4-((1R,3R)-2-(2,2- difluoroethyl)-3-methyl-2,3,4,9- tetrahydro-1H-pyrido[3,4-b]indol-1-yl)- 3,5-difluorophenyl) cyclohexyl)methyl)piperidin- 4-yl)-1-oxo-3,5,6,7- tetrahydropyrrolo[3,4-f]isoindol-2(1H)- yl)piperidine-2,6-dione |
| E 309 | 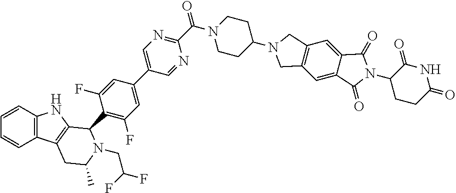 [SMILES: C[C@@H]1Cc2c([nH]c3ccccc23)[C@@H](c2c(F)cc(-c3cnc(C(=O)N4CCC(N5Cc6cc7c(cc6C5)C(=O)N(C5CCC(=O)NC5=O)C7=O)CC4)nc3)cc2F)N1CC(F)F] | 6-(1-(5-(4-((1R,3R)-2-(2,2- difluoroethyl)-3-methyl-2,3,4,9- tetrahydro-1H-pyrido[3,4-b]indol-1-yl)- 3,5-difluorophenyl)pyrimidine-2- carbonyl)piperidin-4-yl)-2-(2,6- dioxopiperidin-3-yl)-6,7- dihydropyrrolo[3,4-f]isoindole- 1,3(2H,5H)-dione |
| E 310 | 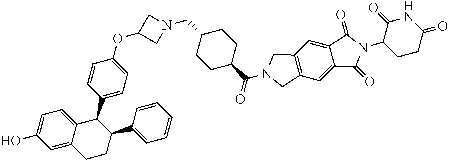 [SMILES: O=C1CCC(N2C(=O)c3cc4c(cc3C2=O)CN(C(=O)[C@H]2CC[C@H](CN3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4r)- 4-((3-(4-((1R,2S)-6-hydroxy-2-phenyl- 1,2,3,4-tetrahydronaphthalen-1- yl)phenoxy)azetidin-1- yl)methyl)cyclohexane-1-carbonyl)-6,7- dihydropyrrolo[3,4-f]isoindole- 1,3(2H,5H)-dione |
| E 311 | 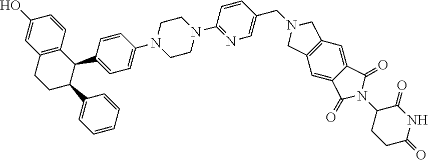 [SMILES: O=C1CCC(N2C(=O)c3cc4c(cc3C2=O)CN(Cc2ccc(N3CCN(c5ccc([C@@H]6c7ccc(O)cc7CC[C@@H]6c6ccccc6)cc5)CC3)nc2)C4)C(=O)N1] | 2-(2,6-dioxopiperidin-3-yl)-6-((6-(4-(4- ((1R,2S)-6-hydroxy-2-phenyl-1,2,3,4- tetrahydronaphthalen-1- yl)phenyl)piperazin-1-yl)pyridin-3- yl)methyl)-6,7-dihydropyrrolo[3,4- f]isoindole-1,3(2H,5H)-dione |
| E 312 | 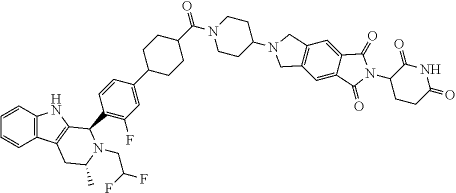 [SMILES: C[C@@H]1Cc2c([nH]c3ccccc23)[C@@H](c2ccc(C3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4-(4-((1R,3R)-2-(2,2- difluoroethyl)-3-methyl-2,3,4,9- tetrahydro-1H-pyrido[3,4-b]indol-1-yl)- 3-fluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 313 | 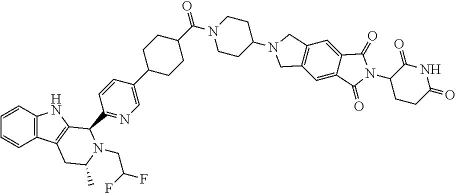 [SMILES: C[C@@H]1Cc2c([nH]c3ccccc23)[C@@H](c2ccc(C3CCC(C(=O)N4CCC(N5Cc6cc7c(cc6C5)C(=O)N(C5CCC(=O)NC5=O)C7=O)CC4)CC3)cn2)N1CC(F)F] | 6-(1-(4-(6-((1S,3R)-2-(2,2- difluoroethyl)-3-methyl-2,3,4,9- tetrahydro-1H-pyrido[3,4-b]indol-1- yl)pyridin-3-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 314 | 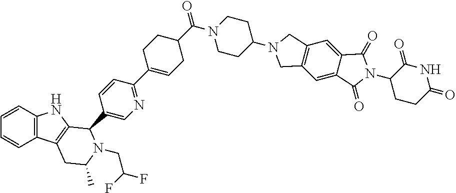 [SMILES: C[C@@H]1Cc2c([nH]c3ccccc23)[C@@H](c2ccc(C3=CCC(C(=O)N4CCC(N5Cc6cc7c(cc6C5)C(=O)N(C5CCC(=O)NC5=O)C7=O)CC4)CC3)nc2)N1CC(F)F] | 6-(1-(4-(5-((1R,3R)-2-(2,2- difluoroethyl)-3-methyl-2,3,4,9- tetrahydro-1H-pyrido[3,4-b]indol-1- yl)pyridin-2-yl)cyclohex-3-ene-1- carbonyl)piperidin-4-yl)-2-(2,6- dioxopiperidin-3-yl)-6,7- dihydropyrrolo[3,4-f]isoindole- 1,3(2H,5H)-dione |
| E 315 | 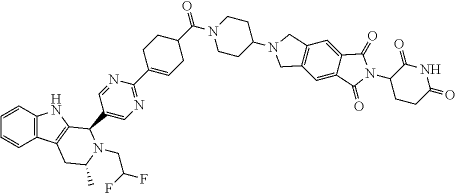 [SMILES: C[C@@H]1Cc2c([nH]c3ccccc23)[C@@H](c2cnc(C3=CCC(C(=O)N4CCC(N5Cc6cc7c(cc6C5)C(=O)N(C5CCC(=O)NC5=O)C7=O)CC4)CC3)nc2)N1CC(F)F] | 6-(1-(4-(5-((1R,3R)-2-(2,2- difluoroethyl)-3-methyl-2,3,4,9- tetrahydro-1H-pyrido[3,4-b]indol-1- yl)pyrimidin-2-yl)cyclohex-3-ene-1- carbonyl)piperidin-4-yl)-2-(2,6- dioxopiperidin-3-yl)-6,7- dihydropyrrolo[3,4-f]isoindole- 1,3(2H,5H)-dione |
| E 316 | 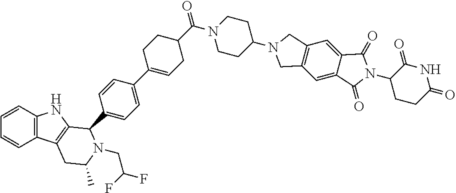 [SMILES: C[C@@H]1Cc2c([nH]c3ccccc23)[C@@H](c2ccc(C3=CCC(C(=O)N4CCC(N5Cc6cc7c(cc6C5)C(=O)N(C5CCC(=O)NC5=O)C7=O)CC4)CC3)cc2)N1CC(F)F] | 6-(1-(4′-((1R,3R)-2-(2,2-difluoroethyl)- 3-methyl-2,3,4,9-tetrahydro-1H- pyrido[3,4-b]indol-1-yl)-2,3,4,5- tetrahydro-[1,1′-biphenyl]-4- carbonyl)piperidin-4-yl)-2-(2,6- dioxopiperidin-3-yl)-6,7- dihydropyrrolo[3,4-f]isoindole- 1,3(2H,5H)-dione |
| E 317 | 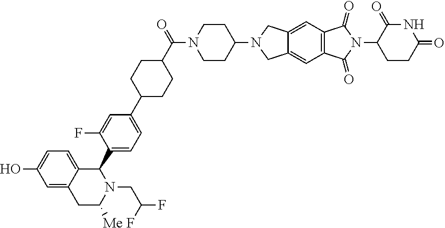 [SMILES: C[C@@H]1Cc2cc(O)ccc2[C@@H](c2ccc(C3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4-(4-((1S,3R)-2-(2,2- difluoroethyl)-6-hydroxy-3-methyl- 1,2,3,4-tetrahydroisoquinolin-1-yl)-3- fluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E318 | 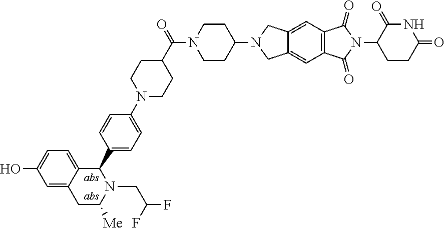 [SMILES: C[C@@H]1Cc2cc(O)ccc2[C@@H](c2ccc(N3CCC(C(=O)N4CCC(N5Cc6cc7c(cc6C5)C(=O)N(C5CCC(=O)NC5=O)C7=O)CC4)CC3)cc2)N1CC(F)F] | 6-(1-(1-(4-((1R,3R)-2-(2,2- difluoroethyl)-6-hydroxy-3-methyl- 1,2,3,4-tetrahydroisoquinolin-1- yl)phenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 319 | 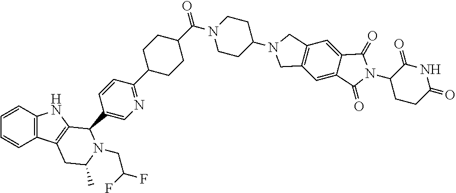 [SMILES: C[C@@H]1Cc2c([nH]c3ccccc23)[C@@H](c2ccc(C3CCC(C(=O)N4CCC(N5Cc6cc7c(cc6C5)C(=O)N(C5CCC(=O)NC5=O)C7=O)CC4)CC3)nc2)N1CC(F)F] | 6-(1-(4-(5-((1R,3R)-2-(2,2- difluoroethyl)-3-methyl-2,3,4,9- tetrahydro-1H-pyrido[3,4-b]indol-1- yl)pyridin-2-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E320 | 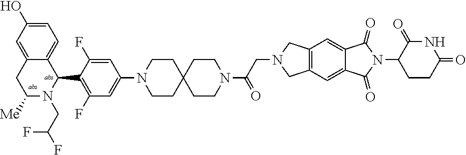 [SMILES: C[C@@H]1Cc2cc(O)ccc2[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)F] | 6-(2-(9-(4-((1S,3R)-2-(2,2- difluoroethyl)-6-hydroxy-3-methyl- 1,2,3,4-tetrahydroisoquinolin-1-yl)-3,5- difluorophenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 321 | 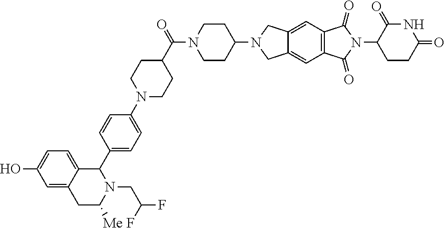 [SMILES: C[C@@H]1Cc2cc(O)ccc2C(c2ccc(N3CCC(C(=O)N4CCC(N5Cc6cc7c(cc6C5)C(=O)N(C5CCC(=O)NC5=O)C7=O)CC4)CC3)cc2)N1CC(F)F] | 6-(1-(1-(4-((3R)-2-(2,2-difluoroethyl)-6- hydroxy-3-methyl-1,2,3,4- tetrahydroisoquinolin-1- yl)phenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E322 | 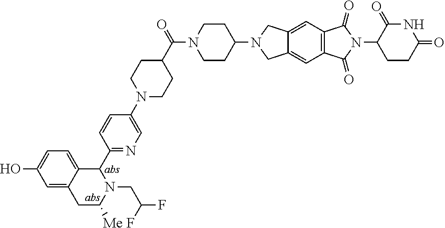 [SMILES: C[C@@H]1Cc2cc(O)ccc2C(c2ccc(N3CCC(C(=O)N4CCC(N5Cc6cc7c(cc6C5)C(=O)N(C5CCC(=O)NC5=O)C7=O)CC4)CC3)cn2)N1CC(F)F] | 6-(1-(1-(6-((3R)-2-(2,2-difluoroethyl)-6- hydroxy-3-methyl-1,2,3,4- tetrahydroisoquinolin-1-yl)pyridin-3- yl)piperidine-4-carbonyl)piperidin-4-yl)- 2-(2,6-dioxopiperidin-3-yl)-6,7- dihydropyrrolo[3,4-f]isoindole- 1,3(2H,5H)-dione |
| E323 | 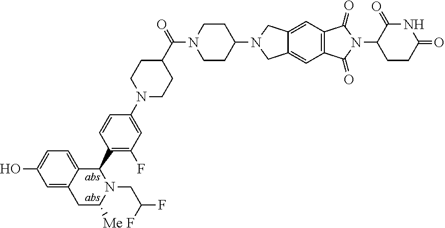 [SMILES: C[C@@H]1Cc2cc(O)ccc2[C@@H](c2ccc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((1S,3R)-2-(2,2- difluoroethyl)-6-hydroxy-3-methyl- 1,2,3,4-tetrahydroisoquinolin-1-yl)-3- fluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 324 | 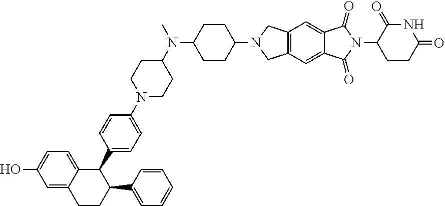 [SMILES: CN(C1CCC(N2Cc3cc4c(cc3C2)C(=O)N(C2CCC(=O)NC2=O)C4=O)CC1)C1CCN(c2ccc([C@@H]3c4ccc(O)cc4CC[C@@H]3c3ccccc3)cc2)CC1] | 2-(2,6-dioxopiperidin-3-yl)-6-(4-((1-(4- ((1R,2S)-6-hydroxy-2-phenyl-1,2,3,4- tetrahydronaphthalen-1- yl)phenyl)piperidin-4- yl)(methyl)amino)cyclohexyl)-6,7- dihydropyrrolo[3,4-f]isoindole- 1,3(2H,5H)-dione |
| E 325 | 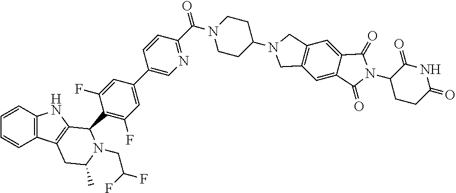 [SMILES: C[C@@H]1Cc2c([nH]c3ccccc23)[C@@H](c2c(F)cc(-c3ccc(C(=O)N4CCC(N5Cc6cc7c(cc6C5)C(=O)N(C5CCC(=O)NC5=O)C7=O)CC4)nc3)cc2F)N1CC(F)F] | 6-(1-(5-(4-((1R,3R)-2-(2,2- difluoroethyl)-3-methyl-2,3,4,9- tetrahydro-1H-pyrido[3,4-b]indol-1-yl)- 3,5-difluorophenyl)picolinoyl)piperidin- 4-yl)-2-(2,6-dioxopiperidin-3-yl)-6,7- dihydropyrrolo[3,4-f]isoindole- 1,3(2H,5H)-dione |
| E326 | 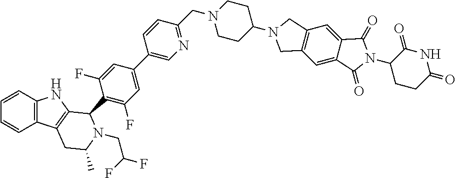 [SMILES: C[C@@H]1Cc2c([nH]c3ccccc23)[C@@H](c2c(F)cc(-c3ccc(CN4CCC(N5Cc6cc7c(cc6C5)C(=O)N(C5CCC(=O)NC5=O)C7=O)CC4)nc3)cc2F)N1CC(F)F] | 6-(1-((5-(4-((1R,3R)-2-(2,2- difluoroethyl)-3-methyl-2,3,4,9- tetrahydro-1H-pyrido[3,4-b]indol-1-yl)- 3,5-difluorophenyl)pyridin-2- yl)methyl)piperidin-4-yl)-2-(2,6- dioxopiperidin-3-yl)-6,7- dihydropyrrolo[3,4-f]isoindole- 1,3(2H,5H)-dione |
| E327 | 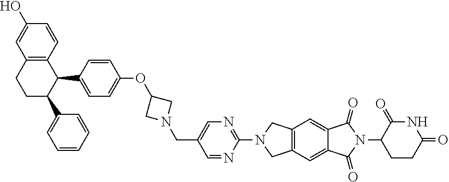 [SMILES: O=C1CCC(N2C(=O)c3cc4c(cc3C2=O)CN(c2ncc(CN3CC(Oc5ccc([C@@H]6c7ccc(O)cc7CC[C@@H]6c6ccccc6)cc5)C3)cn2)C4)C(=O)N1] | 2-(2,6-dioxopiperidin-3-yl)-6-(5-((3-(4- ((1R,2S)-6-hydroxy-2-phenyl-1,2,3,4- tetrahydronaphthalen-1- yl)phenoxy)azetidin-1- yl)methyl)pyrimidin-2-yl)-6,7- dihydropyrrolo[3,4-f]isoindole- 1,3(2H,5H)-dione |
| E328 | 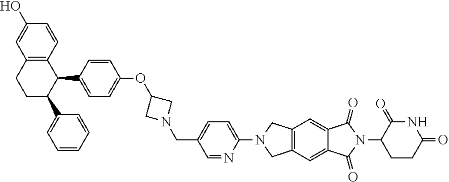 [SMILES: O=C1CCC(N2C(=O)c3cc4c(cc3C2=O)CN(c2ccc(CN3CC(Oc5ccc([C@@H]6c7ccc(O)cc7CC[C@@H]6c6ccccc6)cc5)C3)cn2)C4)C(=O)N1] | 2-(2,6-dioxopiperidin-3-yl)-6-(5-((3-(4- ((1R,2S)-6-hydroxy-2-phenyl-1,2,3,4- tetrahydronaphthalen-1- yl)phenoxy)azetidin-1- yl)methyl)pyridin-2-yl)-6,7- dihydropyrrolo[3,4-f]isoindole- 1,3(2H,5H)-dione |
| E329 | 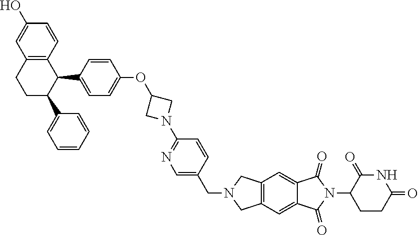 [SMILES: O=C1CCC(N2C(=O)c3cc4c(cc3C2=O)CN(Cc2ccc(N3CC(Oc5ccc([C@@H]6c7ccc(O)cc7CC[C@@H]6c6ccccc6)cc5)C3)nc2)C4)C(=O)N1] | 2-(2,6-dioxopiperidin-3-yl)-6-((6-(3-(4- ((1R,2S)-6-hydroxy-2-phenyl-1,2,3,4- tetrahydronaphthalen-1- yl)phenoxy)azetidin-1-yl)pyridin-3- yl)methyl)-6,7-dihydropyrrolo[3,4- f]isoindole-1,3(2H,5H)-dione |
| E330 | 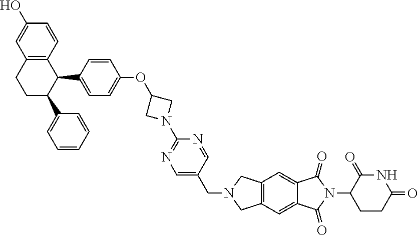 [SMILES: O=C1CCC(N2C(=O)c3cc4c(cc3C2=O)CN(Cc2cnc(N3CC(Oc5ccc([C@@H]6c7ccc(O)cc7CC[C@@H]6c6ccccc6)cc5)C3)nc2)C4)C(=O)N1] | 2-(2,6-dioxopiperidin-3-yl)-6-((2-(3-(4- ((1R,2S)-6-hydroxy-2-phenyl-1,2,3,4- tetrahydronaphthalen-1- yl)phenoxy)azetidin-1-yl)pyrimidin-5- yl)methyl)-6,7-dihydropyrrolo[3,4- f]isoindole-1,3(2H,5H)-dione |
| E331 | 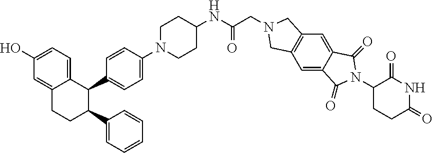 [SMILES: O=C1CCC(N2C(=O)c3cc4c(cc3C2=O)CN(CC(=O)NC2CCN(c3ccc([C@@H]5c6ccc(O)cc6CC[C@@H]5c5ccccc5)cc3)CC2)C4)C(=O)N1] | 2-(6-(2,6-dioxopiperidin-3-yl)-5,7- dioxo-3,5,6,7-tetrahydropyrrolo[3,4- f]isoindol-2(1H)-yl)-N-(1-(4-((1R,2S)- 6-hydroxy-2-phenyl-1,2,3,4- tetrahydronaphthalen-1- yl)phenyl)piperidin-4-yl)acetamide |
| E332 | 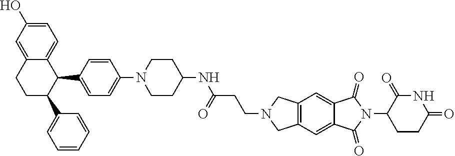 [SMILES: O=C1CCC(N2C(=O)c3cc4c(cc3C2=O)CN(CCC(=O)NC2CCN(c3ccc([C@@H]5c6ccc(O)cc6CC[C@@H]5c5ccccc5)cc3)CC2)C4)C(=O)N1] | 3-(6-(2,6-dioxopiperidin-3-yl)-5,7- dioxo-3,5,6,7-tetrahydropyrrolo[3,4- f]isoindol-2(1H)-yl)-N-(1-(4-((1R,2S)- 6-hydroxy-2-phenyl-1,2,3,4- tetrahydronaphthalen-1- yl)phenyl)piperidin-4-yl)propanamide |
| E333 | 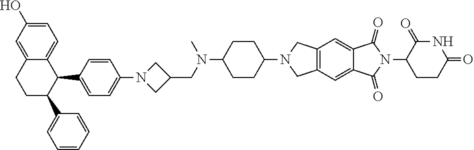 [SMILES: CN(CC1CN(c2ccc([C@@H]3c4ccc(O)cc4CC[C@@H]3c3ccccc3)cc2)C1)C1CCC(N2Cc3cc4c(cc3C2)C(=O)N(C2CCC(=O)NC2=O)C4=O)CC1] | 2-(2,6-dioxopiperidin-3-yl)-6-(4-(((1-(4- ((1R,2S)-6-hydroxy-2-phenyl-1,2,3,4- tetrahydronaphthalen-1- yl)phenyl)azetidin-3- yl)methyl)(methyl)amino)cyclohexyl)- 6,7-dihydropyrrolo[3,4-f]isoindole- 1,3(2H,5H)-dione |
| E334 | 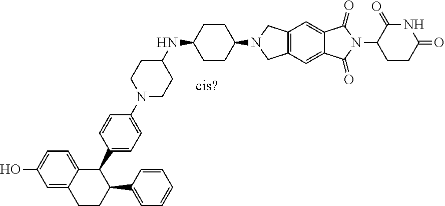 [SMILES: O=C1CCC(N2C(=O)c3cc4c(cc3C2=O)CN([C@H]2CC[C@@H](NC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1S,4s)- 4-((1-(4-((1R,2S)-6-hydroxy-2-phenyl- 1,2,3,4-tetrahydronaphthalen-1- yl)phenyl)piperidin-4- yl)amino)cyclohexyl)-6,7- dihydropyrrolo[3,4-f]isoindole- 1,3(2H,5H)-dione |
| E 335 | 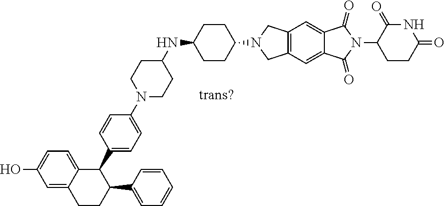 [SMILES: O=C1CCC(N2C(=O)c3cc4c(cc3C2=O)CN([C@H]2CC[C@H](NC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1R,4r)- 4-((1-(4-((1R,2S)-6-hydroxy-2-phenyl- 1,2,3,4-tetrahydronaphthalen-1- yl)phenyl)piperidin-4- yl)amino)cyclohexyl)-6,7- dihydropyrrolo[3,4-f]isoindole- 1,3(2H,5H)-dione |
| E 336 | 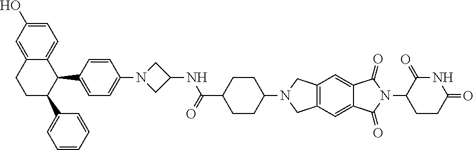 [SMILES: O=C1CCC(N2C(=O)c3cc4c(cc3C2=O)CN(C2CCC(C(=O)NC3CN(c5ccc([C@@H]6c7ccc(O)cc7CC[C@@H]6c6ccccc6)cc5)C3)CC2)C4)C(=O)N1] | 4-(6-(2,6-dioxopiperidin-3-yl)-5,7- dioxo-3,5,6,7-tetrahydropyrrolo[3,4- flisoindol-2(1H)-yl)-N-(1-(4-((1R,2S)- 6-hydroxy-2-phenyl-1,2,3,4- tetrahydronaphthalen-1- yl)phenyl)azetidin-3-yl)cyclohexane-1- carboxamide |
| E 337 | 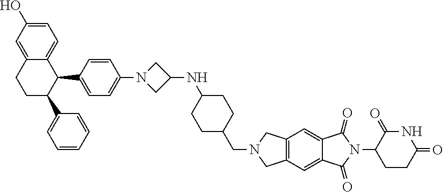 [SMILES: O=C1CCC(N2C(=O)c3cc4c(cc3C2=O)CN(CC2CCC(NC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-((1-(4- ((1R,2S)-6-hydroxy-2-phenyl-1,2,3,4- tetrahydronaphthalen-1- yl)phenyl)azetidin-3- yl)amino)cyclohexyl)methyl)-6,7- dihydropyrrolo[3,4-f]isoindole- 1,3(2H,5H)-dione |
| E 338 | 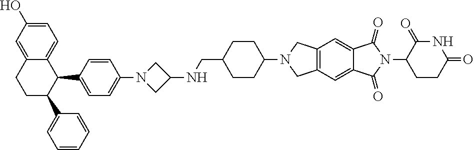 [SMILES: O=C1CCC(N2C(=O)c3cc4c(cc3C2=O)CN(C2CCC(CNC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(4-(((1-(4- ((1R,2S)-6-hydroxy-2-phenyl-1,2,3,4- tetrahydronaphthalen-1- yl)phenyl)azetidin-3- yl)amino)methyl)cyclohexyl)-6,7- dihydropyrrolo[3,4-f]isoindole- 1,3(2H,5H)-dione |
| E 339 | 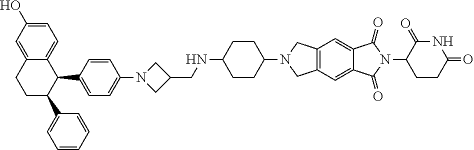 [SMILES: O=C1CCC(N2C(=O)c3cc4c(cc3C2=O)CN(C2CCC(NCC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(4-(((1-(4- ((1R,2S)-6-hydroxy-2-phenyl-1,2,3,4- tetrahydronaphthalen-1- yl)phenyl)azetidin-3- yl)methyl)amino)cyclohexyl)-6,7- dihydropyrrolo[3,4-f]isoindole- 1,3(2H,5H)-dione |
| E 340 | 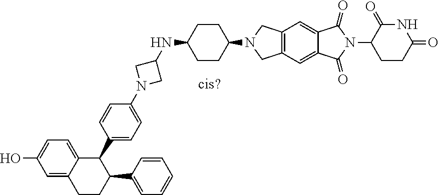 [SMILES: O=C1CCC(N2C(=O)c3cc4c(cc3C2=O)CN([C@H]2CC[C@@H](NC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1S,4s)- 4-((1-(4-((1R,2S)-6-hydroxy-2-phenyl- 1,2,3,4-tetrahydronaphthalen-1- yl)phenyl)azetidin-3- yl)amino)cyclohexyl)-6,7- dihydropyrrolo[3,4-f]isoindole- 1,3(2H,5H)-dione |
| E 341 | 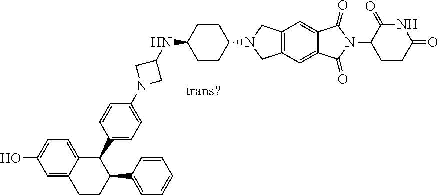 [SMILES: O=C1CCC(N2C(=O)c3cc4c(cc3C2=O)CN([C@H]2CC[C@H](NC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4r)- 4-((1-(4-((1R,2S)-6-hydroxy-2-phenyl- 1,2,3,4-tetrahydronaphthalen-1- yl)phenyl)azetidin-3- yl)amino)cyclohexyl)-6,7- dihydropyrrolo[3,4-f]isoindole- 1,3(2H,5H)-dione |
| E 342 | 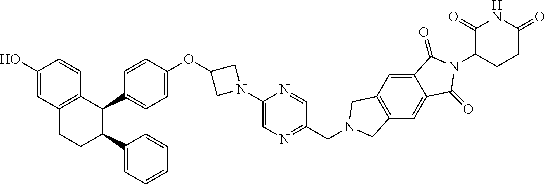 [SMILES: O=C1CCC(N2C(=O)c3cc4c(cc3C2=O)CN(Cc2cnc(N3CC(Oc5ccc([C@@H]6c7ccc(O)cc7CC[C@@H]6c6ccccc6)cc5)C3)cn2)C4)C(=O)N1] | 2-(2,6-dioxopiperidin-3-yl)-6-((5-(3-(4- ((1R,2S)-6-hydroxy-2-phenyl-1,2,3,4- tetrahydronaphthalen-1- yl)phenoxy)azetidin-1-yl)pyrazin-2- yl)methyl)-6,7-dihydropyrrolo[3,4- f]isoindole-1,3(2H,5H)-dione |
| E 343 | 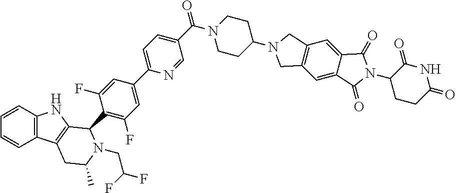 [SMILES: C[C@@H]1Cc2c([nH]c3ccccc23)[C@@H](c2c(F)cc(-c3ccc(C(=O)N4CCC(N5Cc6cc7c(cc6C5)C(=O)N(C5CCC(=O)NC5=O)C7=O)CC4)cn3)cc2F)N1CC(F)F] | 6-(1-(6-(4-((1R,3R)-2-(2,2- difluoroethyl)-3-methyl-2,3,4,9- tetrahydro-1H-pyrido[3,4-b]indol-1-yl)- 3,5-difluorophenyl)nicotinoyl)piperidin- 4-yl)-2-(2,6-dioxopiperidin-3-yl)-6,7- dihydropyrrolo[3,4-f]isoindole- 1,3(2H,5H)-dione |
| E 344 | 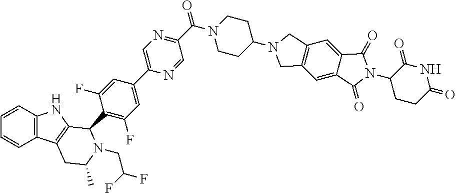 [SMILES: C[C@@H]1Cc2c([nH]c3ccccc23)[C@@H](c2c(F)cc(-c3cnc(C(=O)N4CCC(N5Cc6cc7c(cc6C5)C(=O)N(C5CCC(=O)NC5=O)C7=O)CC4)cn3)cc2F)N1CC(F)F] | 6-(1-(5-(4-((1R,3R)-2-(2,2- difluoroethyl)-3-methyl-2,3,4,9- tetrahydro-1H-pyrido[3,4-b]indol-1-yl)- 3,5-difluorophenyl)pyrazine-2- carbonyl)piperidin-4-yl)-2-(2,6- dioxopiperidin-3-yl)-6,7- dihydropyrrolo[3,4-f]isoindole- 1,3(2H,5H)-dione |
| E 345 | 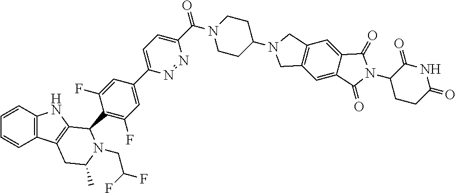 [SMILES: C[C@@H]1Cc2c([nH]c3ccccc23)[C@@H](c2c(F)cc(-c3ccc(C(=O)N4CCC(N5Cc6cc7c(cc6C5)C(=O)N(C5CCC(=O)NC5=O)C7=O)CC4)nn3)cc2F)N1CC(F)F] | 6-(1-(6-(4-((1R,3R)-2-(2,2- difluoroethyl)-3-methyl-2,3,4,9- tetrahydro-1H-pyrido[3,4-b]indol-1-yl)- 3,5-difluorophenyl)pyridazine-3- carbonyl)piperidin-4-yl)-2-(2,6- dioxopiperidin-3-yl)-6,7- dihydropyrrolo[3,4-f]isoindole- 1,3(2H,5H)-dione |
| E 346 | 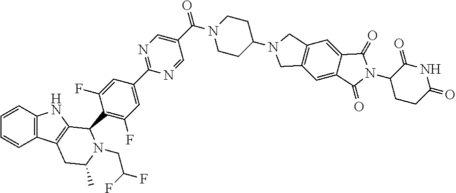 [SMILES: C[C@@H]1Cc2c([nH]c3ccccc23)[C@@H](c2c(F)cc(-c3ncc(C(=O)N4CCC(N5Cc6cc7c(cc6C5)C(=O)N(C5CCC(=O)NC5=O)C7=O)CC4)cn3)cc2F)N1CC(F)F] | 6-(1-(2-(4-((1R,3R)-2-(2,2- difluoroethyl)-3-methyl-2,3,4,9- tetrahydro-1H-pyrido[3,4-b]indol-1-yl)- 3,5-difluorophenyl)pyrimidine-5- carbonyl)piperidin-4-yl)-2-(2,6- dioxopiperidin-3-yl)-6,7- dihydropyrrolo[3,4-f]isoindole- 1,3(2H,5H)-dione |
| E 347 | 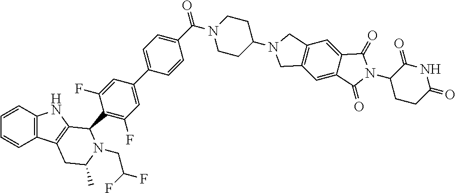 [SMILES: C[C@@H]1Cc2c([nH]c3ccccc23)[C@@H](c2c(F)cc(-c3ccc(C(=O)N4CCC(N5Cc6cc7c(cc6C5)C(=O)N(C5CCC(=O)NC5=O)C7=O)CC4)cc3)cc2F)N1CC(F)F] | 6-(1-(4′-((1R,3R)-2-(2,2-difluoroethyl)- 3-methyl-2,3,4,9-tetrahydro-1H- pyrido[3,4-b]indol-1-yl)-3′,5′-difluoro- [1,1′-biphenyl]-4-carbonyl)piperidin-4- yl)-2-(2,6-dioxopiperidin-3-yl)-6,7- dihydropyrrolo[3,4-f]isoindole- 1,3(2H,5H)-dione |
| E 348 | 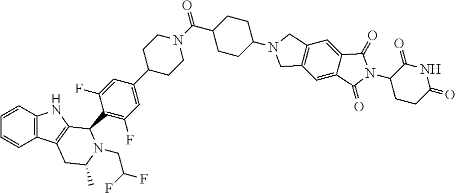 [SMILES: C[C@@H]1Cc2c([nH]c3ccccc23)[C@@H](c2c(F)cc(C3CCN(C(=O)C4CCC(N5Cc6cc7c(cc6C5)C(=O)N(C5CCC(=O)NC5=O)C7=O)CC4)CC3)cc2F)N1CC(F)F] | 6-(4-(4-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| E349 | 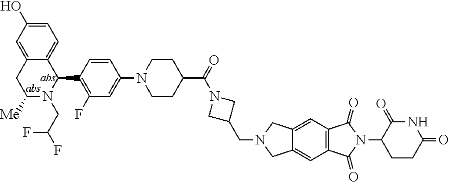 [SMILES: C[C@@H]1Cc2cc(O)ccc2[C@@H](c2ccc(N3CCC(C(=O)N4CC(CN5Cc6cc7c(cc6C5)C(=O)N(C5CCC(=O)NC5=O)C7=O)C4)CC3)cc2F)N1CC(F)F] | 6-((1-(1-(4-((1S,3R)-2-(2,2- difluoroethyl)-6-hydroxy-3-methyl- 1,2,3,4-tetrahydroisoquinolin-1-yl)-3- fluorophenyl)piperidine-4- carbonyl)azetidin-3-yl)methyl)-2-(2,6- dioxopiperidin-3-yl)-6,7- dihydropyrrolo[3,4-f]isoindole- 1,3(2H,5H)-dione |
| E350 | 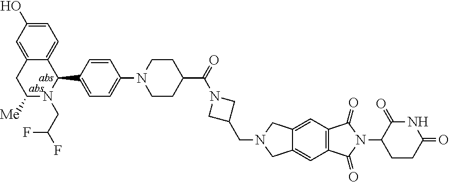 [SMILES: C[C@@H]1Cc2cc(O)ccc2[C@@H](c2ccc(N3CCC(C(=O)N4CC(CN5Cc6cc7c(cc6C5)C(=O)N(C5CCC(=O)NC5=O)C7=O)C4)CC3)cc2)N1CC(F)F] | 6-((1-(1-(4-((1R,3R)-2-(2,2- difluoroethyl)-6-hydroxy-3-methyl- 1,2,3,4-tetrahydroisoquinolin-1- yl)phenyl)piperidine-4- carbonyl)azetidin-3-yl)methyl)-2-(2,6- dioxopiperidin-3-yl)-6,7- dihydropyrrolo[3,4-f]isoindole- 1,3(2H,5H)-dione |
| E351 | 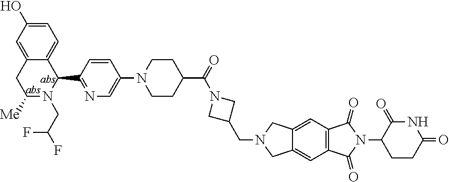 [SMILES: C[C@@H]1Cc2cc(O)ccc2[C@@H](c2ccc(N3CCC(C(=O)N4CC(CN5Cc6cc7c(cc6C5)C(=O)N(C5CCC(=O)NC5=O)C7=O)C4)CC3)cn2)N1CC(F)F] | 6-((1-(1-(6-((1S,3R)-2-(2,2- difluoroethyl)-6-hydroxy-3-methyl- 1,2,3,4-tetrahydroisoquinolin-1- yl)pyridin-3-yl)piperidine-4- carbonyl)azetidin-3-yl)methyl)-2-(2,6- dioxopiperidin-3-yl)-6,7- dihydropyrrolo[3,4-f]isoindole- 1,3(2H,5H)-dione |
| E352 | 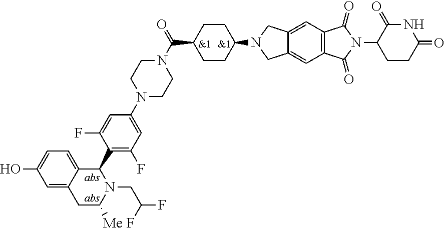 [SMILES: C[C@@H]1Cc2cc(O)ccc2[C@@H](c2c(F)cc(N3CCN(C(=O)[C@H]4CC[C@@H](N5Cc6cc7c(cc6C5)C(=O)N(C5CCC(=O)NC5=O)C7=O)CC4)CC3)cc2F)N1CC(F)F] | Rac-6-((1R,4s)-4-(4-(4-((1S,3R)-2-(2,2- difluoroethyl)-6-hydroxy-3-methyl- 1,2,3,4-tetrahydroisoquinolin-1-yl)-3,5- difluorophenyl)piperazine-1- carbonyl)cyclohexyl)-2-(2,6- dioxopiperidin-3-yl)-6,7- dihydropyrrolo[3,4-f]isoindole- 1,3(2H,5H)-dione |
| E353 | 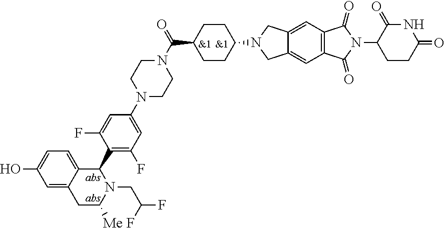 [SMILES: C[C@@H]1Cc2cc(O)ccc2[C@@H](c2c(F)cc(N3CCN(C(=O)[C@H]4CC[C@H](N5Cc6cc7c(cc6C5)C(=O)N(C5CCC(=O)NC5=O)C7=O)CC4)CC3)cc2F)N1CC(F)F] | rac-6-((1S,4r)-4-(4-(4-((1S,3R)-2-(2,2- difluoroethyl)-6-hydroxy-3-methyl- 1,2,3,4-tetrahydroisoquinolin-1-yl)-3,5- difluorophenyl)piperazine-1- carbonyl)cyclohexyl)-2-(2,6- dioxopiperidin-3-yl)-6,7- dihydropyrrolo[3,4-f]isoindole- 1,3(2H,5H)-dione |
| E 354 | 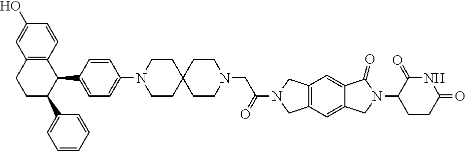 [SMILES: O=C1CCC(N2Cc3cc4c(cc3C2=O)CN(C(=O)CN2CCC3(CC2)CCN(c2ccc([C@@H]5c6ccc(O)cc6CC[C@@H]5c5ccccc5)cc2)CC3)C4)C(=O)N1] | 3-(6-(2-(9-(4-((1R,2S)-6-hydroxy-2- phenyl-1,2,3,4-tetrahydronaphthalen-1- yl)phenyl)-3,9-diazaspiro[5.5]undecan- 3-yl)acetyl)-1-oxo-3,5,6,7- tetrahydropyrrolo[3,4-f]isoindol-2(1H)- yl)piperidine-2,6-dione |
| E 355 | 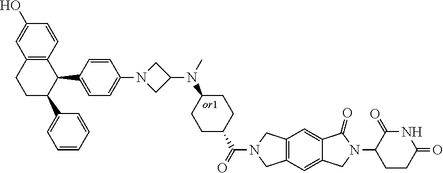 [SMILES: CN(C1CN(c2ccc([C@@H]3c4ccc(O)cc4CC[C@@H]3c3ccccc3)cc2)C1)[C@H]1CC[C@H](C(=O)N2Cc3cc4c(cc3C2)C(=O)N(C2CCC(=O)NC2=O)C4)CC1] | 3-(6-(4-((1-(4-((1R,2S)-6-hydroxy-2- phenyl-1,2,3,4-tetrahydronaphthalen-1- yl)phenyl)azetidin-3- yl)(methyl)amino)cyclohexane-1- carbonyl)-1-oxo-3,5,6,7- tetrahydropyrrolo[3,4-f]isoindol-2(1H)- yl)piperidine-2,6-dione |
| E 356 | 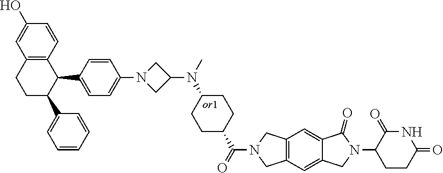 [SMILES: CN(C1CN(c2ccc([C@@H]3c4ccc(O)cc4CC[C@@H]3c3ccccc3)cc2)C1)[C@H]1CC[C@@H](C(=O)N2Cc3cc4c(cc3C2)C(=O)N(C2CCC(=O)NC2=O)C4)CC1] | 3-(6-(4-((1-(4-((1R,2S)-6-hydroxy-2- phenyl-1,2,3,4-tetrahydronaphthalen-1- yl)phenyl)azetidin-3- yl)(methyl)amino)cyclohexane-1- carbonyl)-1-oxo-3,5,6,7- tetrahydropyrrolo[3,4-f]isoindol-2(1H)- yl)piperidine-2,6-dione |
| E 357 | 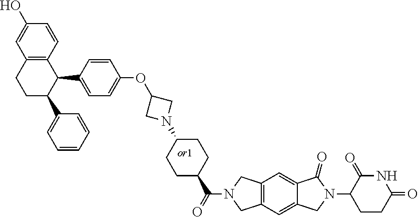 [SMILES: O=C1CCC(N2Cc3cc4c(cc3C2=O)CN(C(=O)[C@H]2CC[C@H](N3CC(Oc5ccc([C@@H]6c7ccc(O)cc7CC[C@@H]6c6ccccc6)cc5)C3)CC2)C4)C(=O)N1] | 3-(6-(4-(3-(4-((1R,2S)-6-hydroxy-2- phenyl-1,2,3,4-tetrahydronaphthalen-1- yl)phenoxy)azetidin-1-yl)cyclohexane- 1-carbonyl)-1-oxo-3,5,6,7- tetrahydropyrrolo[3,4-f]isoindol-2(1H)- yl)piperidine-2,6-dione |
| E 358 | 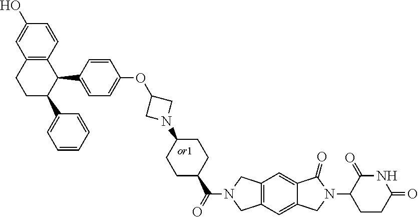 [SMILES: O=C1CCC(N2Cc3cc4c(cc3C2=O)CN(C(=O)[C@H]2CC[C@@H](N3CC(Oc5ccc([C@@H]6c7ccc(O)cc7CC[C@@H]6c6ccccc6)cc5)C3)CC2)C4)C(=O)N1] | 3-(6-(4-(3-(4-((1R,2S)-6-hydroxy-2- phenyl-1,2,3,4-tetrahydronaphthalen-1- yl)phenoxy)azetidin-1-yl)cyclohexane- 1-carbonyl)-1-oxo-3,5,6,7- tetrahydropyrrolo[3,4-f]isoindol-2(1H)- yl)piperidine-2,6-dione |
| E 359 | 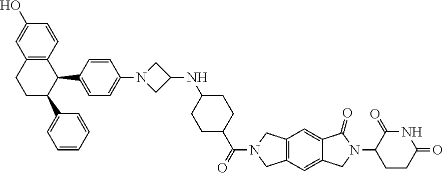 [SMILES: O=C1CCC(N2Cc3cc4c(cc3C2=O)CN(C(=O)C2CCC(NC3CN(c5ccc([C@@H]6c7ccc(O)cc7CC[C@@H]6c6ccccc6)cc5)C3)CC2)C4)C(=O)N1] | 3-(6-(4-((1-(4-((1R,2S)-6-hydroxy-2- phenyl-1,2,3,4-tetrahydronaphthalen-1- yl)phenyl)azetidin-3- yl)amino)cyclohexane-1-carbonyl)-1- oxo-3,5,6,7-tetrahydropyrrolo[3,4- f]isoindol-2(1H)-yl)piperidine-2,6-dione |
| E 360 | 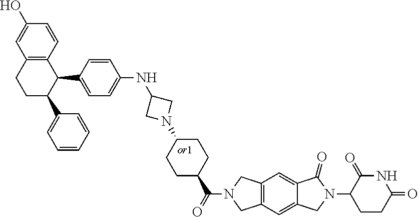 [SMILES: O=C1CCC(N2Cc3cc4c(cc3C2=O)CN(C(=O)[C@H]2CC[C@H](N3CC(Nc5ccc([C@@H]6c7ccc(O)cc7CC[C@@H]6c6ccccc6)cc5)C3)CC2)C4)C(=O)N1] | 3-(6-(4-(3-((4-((1R,2S)-6-hydroxy-2- phenyl-1,2,3,4-tetrahydronaphthalen-1- yl)phenyl)amino)azetidin-1- yl)cyclohexane-1-carbonyl)-1-oxo- 3,5,6,7-tetrahydropyrrolo[3,4-f]isoindol- 2(1H)-yl)piperidine-2,6-dione |
| E 361 | 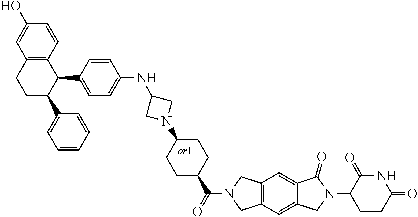 [SMILES: O=C1CCC(N2Cc3cc4c(cc3C2=O)CN(C(=O)[C@H]2CC[C@@H](N3CC(Nc5ccc([C@@H]6c7ccc(O)cc7CC[C@@H]6c6ccccc6)cc5)C3)CC2)C4)C(=O)N1] | 3-(6-(4-(3-((4-((1R,2S)-6-hydroxy-2- phenyl-1,2,3,4-tetrahydronaphthalen-1- yl)phenyl)amino)azetidin-1- yl)cyclohexane-1-carbonyl)-1-oxo- 3,5,6,7-tetrahydropyrrolo[3,4-f]isoindol- 2(1H)-yl)piperidine-2,6-dione |
| E 362 | 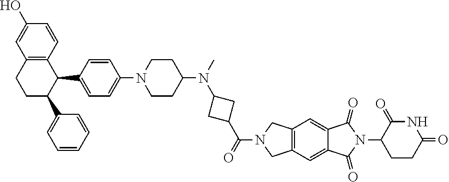 [SMILES: CN(C1CCN(c2ccc([C@@H]3c4ccc(O)cc4CC[C@@H]3c3ccccc3)cc2)CC1)C1CC(C(=O)N2Cc3cc4c(cc3C2)C(=O)N(C2CCC(=O)NC2=O)C4=O)C1] | 2-(2,6-dioxopiperidin-3-yl)-6-(3-((1-(4- ((1R,2S)-6-hydroxy-2-phenyl-1,2,3,4- tetrahydronaphthalen-1- yl)phenyl)piperidin-4- yl)(methyl)amino)cyclobutane-1- carbonyl)-6,7-dihydropyrrolo[3,4- f]isoindole-1,3(2H,5H)-dione |
| E 363 | 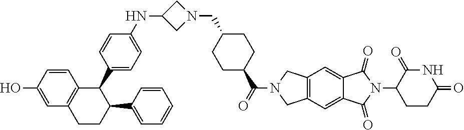 [SMILES: O=C1CCC(N2C(=O)c3cc4c(cc3C2=O)CN(C(=O)[C@H]2CC[C@H](CN3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4r)- 4-((3-((4-((1R,2S)-6-hydroxy-2-phenyl- 1,2,3,4-tetrahydronaphthalen-1- yl)phenyl)amino)azetidin-1- yl)methyl)cyclohexane-1-carbonyl)-6,7- dihydropyrrolo[3,4-f]isoindole- 1,3(2H,5H)-dione |
| E 364 | 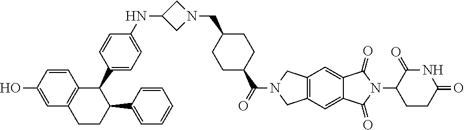 [SMILES: O=C1CCC(N2C(=O)c3cc4c(cc3C2=O)CN(C(=O)[C@H]2CC[C@@H](CN3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1S,4s)- 4-((3-((4-((1R,2S)-6-hydroxy-2-phenyl- 1,2,3,4-tetrahydronaphthalen-1- yl)phenyl)amino)azetidin-1- yl)methyl)cyclohexane-1-carbonyl)-6,7- dihydropyrrolo[3,4-f]isoindole- 1,3(2H,5H)-dione |
| E 365 | 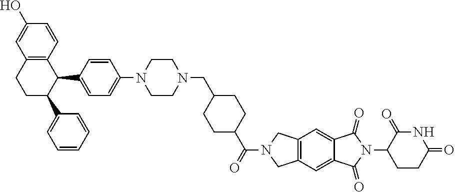 [SMILES: O=C1CCC(N2C(=O)c3cc4c(cc3C2=O)CN(C(=O)C2CCC(CN3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-((4-(4- ((1R,2S)-6-hydroxy-2-phenyl-1,2,3,4- tetrahydronaphthalen-1- yl)phenyl)piperazin-1- yl)methyl)cyclohexane-1-carbonyl)-6,7- dihydropyrrolo[3,4-f]isoindole- 1,3(2H,5H)-dione |
| E 366 | 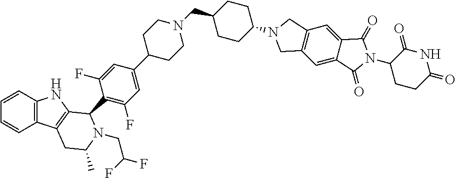 [SMILES: C[C@@H]1Cc2c([nH]c3ccccc23)[C@@H](c2c(F)cc(C3CCN(C[C@H]4CC[C@H](N5Cc6cc7c(cc6C5)C(=O)N(C5CCC(=O)NC5=O)C7=O)CC4)CC3)cc2F)N1CC(F)F] | 6-((1R,4r)-4-((4-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-1- yl)methyl)cyclohexyl)-2-(2,6- dioxopiperidin-3-yl)-6,7- dihydropyrrolo[3,4-f]isoindole- 1,3(2H,5H)-dione |
| E 367 | 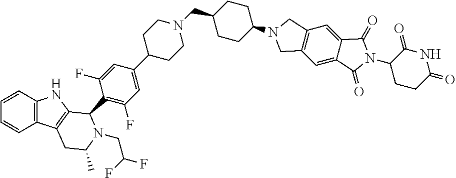 [SMILES: C[C@@H]1Cc2c([nH]c3ccccc23)[C@@H](c2c(F)cc(C3CCN(C[C@H]4CC[C@@H](N5Cc6cc7c(cc6C5)C(=O)N(C5CCC(=O)NC5=O)C7=O)CC4)CC3)cc2F)N1CC(F)F] | 6-((1S,4s)-4-((4-(4-((1R,3R)-2-(2,2- difluoroethyl)-3-methyl-2,3,4,9- tetrahydro-1H-pyrido[3,4-b]indol-1-yl)- 3,5-difluorophenyl)piperidin-1- yl)methyl)cyclohexyl)-2-(2,6- dioxopiperidin-3-yl)-6,7- dihydropyrrolo[3,4-f]isoindole- 1,3(2H,5H)-dione |
| E 368 | 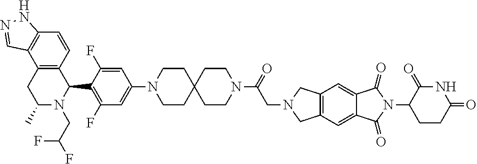 [SMILES: C[C@@H]1Cc2c(ccc3[nH]ncc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)F] | 6-(2-(9-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 3,9-diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 369 | 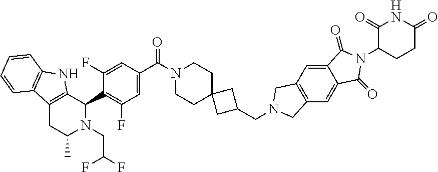 [SMILES: C[C@@H]1Cc2c([nH]c3ccccc23)[C@@H](c2c(F)cc(C(=O)N3CCC4(CC3)CC(CN3Cc5cc6c(cc5C3)C(=O)N(C3CCC(=O)NC3=O)C6=O)C4)cc2F)N1CC(F)F] | 6-((7-(4-((1R,3R)-2-(2,2-difluoroethyl)- 3-methyl-2,3,4,9-tetrahydro-1H- pyrido[3,4-b]indol-1-yl)-3,5- difluorobenzoyl)-7-azaspiro[3.5]nonan- 2-yl)methyl)-2-(2,6-dioxopiperidin-3- yl)-6,7-dihydropyrrolo[3,4-f]isoindole- 1,3(2H,5H)-dione |
| E 370 | 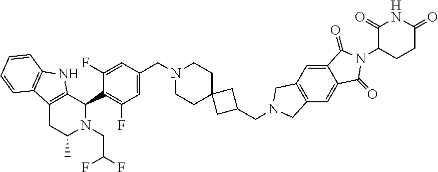 [SMILES: C[C@@H]1Cc2c([nH]c3ccccc23)[C@@H](c2c(F)cc(CN3CCC4(CC3)CC(CN3Cc5cc6c(cc5C3)C(=O)N(C3CCC(=O)NC3=O)C6=O)C4)cc2F)N1CC(F)F] | 6-((7-(4-((1R,3R)-2-(2,2-difluoroethyl)- 3-methyl-2,3,4,9-tetrahydro-1H- pyrido[3,4-b]indol-1-yl)-3,5- difluorobenzyl)-7-azaspiro[3.5]nonan-2- yl)methyl)-2-(2,6-dioxopiperidin-3-yl)- 6,7-dihydropyrrolo[3,4-f]isoindole- 1,3(2H,5H)-dione |
| E 371 | 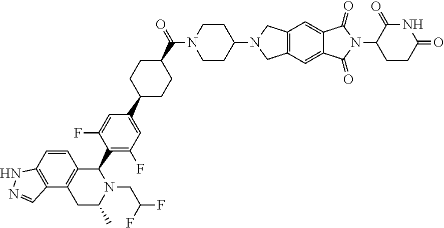 [SMILES: C[C@@H]1Cc2c(ccc3[nH]ncc23)[C@@H](c2c(F)cc([C@H]3CC[C@@H](C(=O)N4CCC(N5Cc6cc7c(cc6C5)C(=O)N(C5CCC(=O)NC5=O)C7=O)CC4)CC3)cc2F)N1CC(F)F] | 6-(1-((1R,4s)-4-(4-((6S,8R)-7-(2,2- difluoroethyl)-8-methyl-6,7,8,9- tetrahydro-3H-pyrazolo[4,3- flisoquinolin-6-yl)-3,5- difluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 372 | 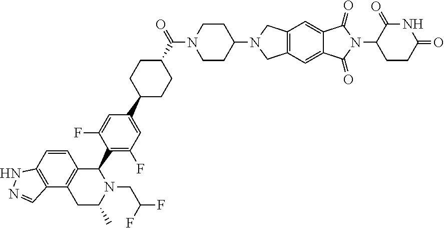 [SMILES: C[C@@H]1Cc2c(ccc3[nH]ncc23)[C@@H](c2c(F)cc([C@H]3CC[C@H](C(=O)N4CCC(N5Cc6cc7c(cc6C5)C(=O)N(C5CCC(=O)NC5=O)C7=O)CC4)CC3)cc2F)N1CC(F)F] | 6-(1-((1S,4r)-4-(4-((6S,8R)-7-(2,2- difluoroethyl)-8-methyl-6,7,8,9- tetrahydro-3H-pyrazolo[4,3- flisoquinolin-6-yl)-3,5- difluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 373 | 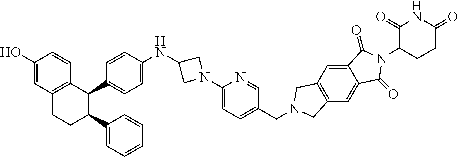 [SMILES: O=C1CCC(N2C(=O)c3cc4c(cc3C2=O)CN(Cc2ccc(N3CC(Nc5ccc([C@@H]6c7ccc(O)cc7CC[C@@H]6c6ccccc6)cc5)C3)nc2)C4)C(=O)N1] | 2-(2,6-dioxopiperidin-3-yl)-6-((6-(3-((4- ((1R,2S)-6-hydroxy-2-phenyl-1,2,3,4- tetrahydronaphthalen-1- yl)phenyl)amino)azetidin-1-yl)pyridin- 3-yl)methyl)-6,7-dihydropyrrolo[3,4- f]isoindole-1,3(2H,5H)-dione |
| E 374 | 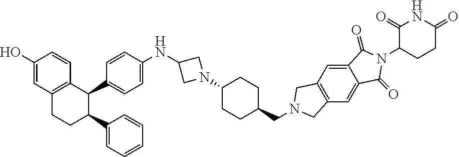 [SMILES: O=C1CCC(N2C(=O)c3cc4c(cc3C2=O)CN(C[C@H]2CC[C@H](N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-(((1R,4r)- 4-(3-((4-((1R,2S)-6-hydroxy-2-phenyl- 1,2,3,4-tetrahydronaphthalen-1- yl)phenyl)amino)azetidin-1- yl)cyclohexyl)methyl)-6,7- dihydropyrrolo[3,4-f]isoindole- 1,3(2H,5H)-dione |
| E 375 | 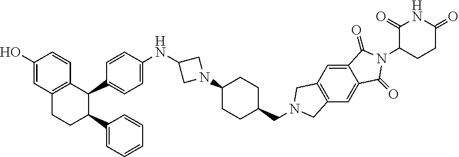 [SMILES: O=C1CCC(N2C(=O)c3cc4c(cc3C2=O)CN(C[C@H]2CC[C@@H](N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-(((1S,4s)- 4-(3-((4-((1R,2S)-6-hydroxy-2-phenyl- 1,2,3,4-tetrahydronaphthalen-1- yl)phenyl)amino)azetidin-1- yl)cyclohexyl)methyl)-6,7- dihydropyrrolo[3,4-f]isoindole- 1,3(2H,5H)-dione |
| E 376 | 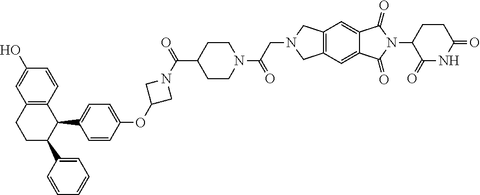 [SMILES: O=C1CCC(N2C(=O)c3cc4c(cc3C2=O)CN(CC(=O)N2CCC(C(=O)N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(2-(4-(3- (4-((1R,2S)-6-hydroxy-2-phenyl- 1,2,3,4-tetrahydronaphthalen-1- yl)phenoxy)azetidine-1- carbonyl)piperidin-1-yl)-2-oxoethyl)- 6,7-dihydropyrrolo[3,4-f]isoindole- 1,3(2H,5H)-dione |
| E 377 | 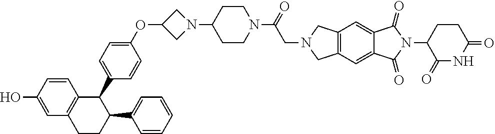 [SMILES: O=C1CCC(N2C(=O)c3cc4c(cc3C2=O)CN(CC(=O)N2CCC(N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(2-(4-(3- (4-((1R,2S)-6-hydroxy-2-phenyl- 1,2,3,4-tetrahydronaphthalen-1- yl)phenoxy)azetidin-1-yl)piperidin-1- yl)-2-oxoethyl)-6,7-dihydropyrrolo[3,4- f]isoindole-1,3(2H,5H)-dione |
| E 378 | 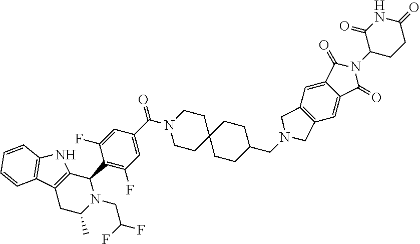 [SMILES: C[C@@H]1Cc2c([nH]c3ccccc23)[C@@H](c2c(F)cc(C(=O)N3CCC4(CCC(CN5Cc6cc7c(cc6C5)C(=O)N(C5CCC(=O)NC5=O)C7=O)CC4)CC3)cc2F)N1CC(F)F] | 6-((3-(4-((1R,3R)-2-(2,2-difluoroethyl)- 3-methyl-2,3,4,9-tetrahydro-1H- pyrido[3,4-b]indol-1-yl)-3,5- difluorobenzoyl)-3- azaspiro[5.5]undecan-9-yl)methyl)-2- (2,6-dioxopiperidin-3-yl)-6,7- dihydropyrrolo[3,4-f]isoindole- 1,3(2H,5H)-dione |
| E 379 | 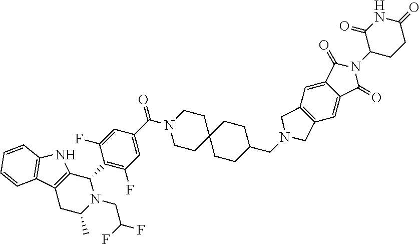 [SMILES: C[C@@H]1Cc2c([nH]c3ccccc23)[C@H](c2c(F)cc(C(=O)N3CCC4(CCC(CN5Cc6cc7c(cc6C5)C(=O)N(C5CCC(=O)NC5=O)C7=O)CC4)CC3)cc2F)N1CC(F)F] | 6-((3-(4-((1S,3R)-2-(2,2-difluoroethyl)- 3-methyl-2,3,4,9-tetrahydro-1H- pyrido[3,4-b]indol-1-yl)-3,5- difluorobenzoyl)-3- azaspiro[5.5]undecan-9-yl)methyl)-2- (2,6-dioxopiperidin-3-yl)-6,7- dihydropyrrolo[3,4-f]isoindole- 1,3(2H,5H)-dione |
| E 380 | 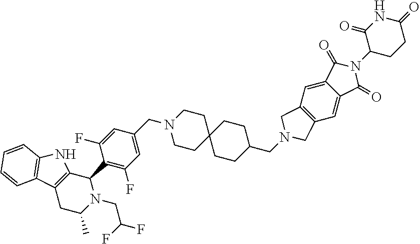 [SMILES: C[C@@H]1Cc2c([nH]c3ccccc23)[C@@H](c2c(F)cc(CN3CCC4(CCC(CN5Cc6cc7c(cc6C5)C(=O)N(C5CCC(=O)NC5=O)C7=O)CC4)CC3)cc2F)N1CC(F)F] | 6-((3-(4-((1R,3R)-2-(2,2-difluoroethyl)- 3-methyl-2,3,4,9-tetrahydro-1H- pyrido[3,4-b]indol-1-yl)-3,5- difluorobenzyl)-3-azaspiro[5.5]undecan- 9-yl)methyl)-2-(2,6-dioxopiperidin-3- yl)-6,7-dihydropyrrolo[3,4-f]isoindole- 1,3(2H,5H)-dione |
| E 381 | 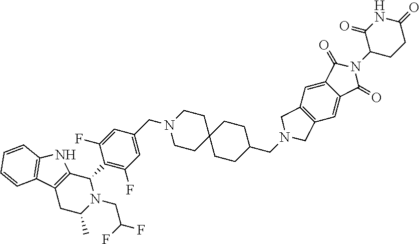 [SMILES: C[C@@H]1Cc2c([nH]c3ccccc23)[C@H](c2c(F)cc(CN3CCC4(CCC(CN5Cc6cc7c(cc6C5)C(=O)N(C5CCC(=O)NC5=O)C7=O)CC4)CC3)cc2F)N1CC(F)F] | 6-((3-(4-((1S,3R)-2-(2,2-difluoroethyl)- 3-methyl-2,3,4,9-tetrahydro-1H- pyrido[3,4-b]indol-1-yl)-3,5- difluorobenzyl)-3-azaspiro[5.5]undecan- 9-yl)methyl)-2-(2,6-dioxopiperidin-3- yl)-6,7-dihydropyrrolo[3,4-f]isoindole- 1,3(2H,5H)-dione |
| E 382 | 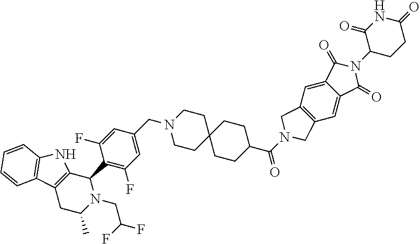 [SMILES: C[C@@H]1Cc2c([nH]c3ccccc23)[C@@H](c2c(F)cc(CN3CCC4(CCC(C(=O)N5Cc6cc7c(cc6C5)C(=O)N(C5CCC(=O)NC5=O)C7=O)CC4)CC3)cc2F)N1CC(F)F] | 6-(3-(4-((1R,3R)-2-(2,2-difluoroethyl)- 3-methyl-2,3,4,9-tetrahydro-1H- pyrido[3,4-b]indol-1-yl)-3,5- difluorobenzyl)-3- azaspiro[5.5]undecane-9-carbonyl)-2- (2,6-dioxopiperidin-3-yl)-6,7- dihydropyrrolo[3,4-f]isoindole- 1,3(2H,5H)-dione |
| E 383 | 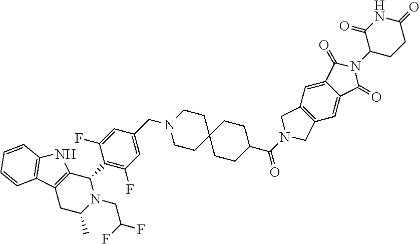 [SMILES: C[C@@H]1Cc2c([nH]c3ccccc23)[C@H](c2c(F)cc(CN3CCC4(CCC(C(=O)N5Cc6cc7c(cc6C5)C(=O)N(C5CCC(=O)NC5=O)C7=O)CC4)CC3)cc2F)N1CC(F)F] | 6-(3-(4-((1S,3R)-2-(2,2-difluoroethyl)- 3-methyl-2,3,4,9-tetrahydro-1H- pyrido[3,4-b]indol-1-yl)-3,5- difluorobenzyl)-3- azaspiro[5.5]undecane-9-carbonyl)-2- (2,6-dioxopiperidin-3-yl)-6,7- dihydropyrrolo[3,4-f]isoindole- 1,3(2H,5H)-dione |
| E 384 | 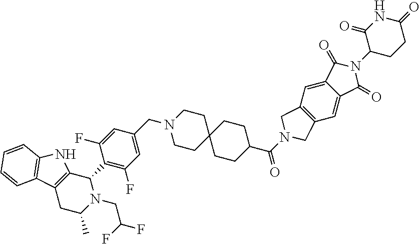 [SMILES: C[C@@H]1Cc2c([nH]c3ccccc23)[C@H](c2c(F)cc(CN3CCC4(CCC(C(=O)N5Cc6cc7c(cc6C5)C(=O)N(C5CCC(=O)NC5=O)C7=O)CC4)CC3)cc2F)N1CC(F)F] | 2-(2,6-dioxopiperidin-3-yl)-6-((1R,4s)- 4-(3-(4-((1S,2R)-6-hydroxy-2-phenyl- 1,2,3,4-tetrahydronaphthalen-1- yl)phenoxy)azetidine-1- carbonyl)cyclohexyl)-6,7- dihydropyrrolo[3,4-f]isoindole- 1,3(2H,5H)-dione |
| E 385 | 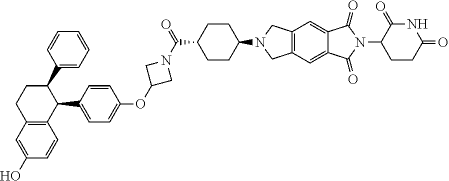 [SMILES: O=C1CCC(N2C(=O)c3cc4c(cc3C2=O)CN([C@H]2CC[C@H](C(=O)N3CC(Oc5ccc([C@H]6c7ccc(O)cc7CC[C@H]6c6ccccc6)cc5)C3)CC2)C4)C(=O)N1] | 2-(2,6-dioxopiperidin-3-yl)-6-((1S,4r)-4- (3-(4-((1S,2R)-6-hydroxy-2-phenyl- 1,2,3,4-tetrahydronaphthalen-1- yl)phenoxy)azetidine-1- carbonyl)cyclohexyl)-6,7- dihydropyrrolo[3,4-f]isoindole- 1,3(2H,5H)-dione |
| E 386 | 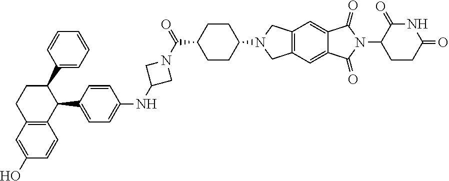 [SMILES: O=C1CCC(N2C(=O)c3cc4c(cc3C2=O)CN([C@H]2CC[C@@H](C(=O)N3CC(Nc5ccc([C@H]6c7ccc(O)cc7CC[C@H]6c6ccccc6)cc5)C3)CC2)C4)C(=O)N1] | 2-(2,6-dioxopiperidin-3-yl)-6-((1R,4s)- 4-(3-((4-((1S,2R)-6-hydroxy-2-phenyl- 1,2,3,4-tetrahydronaphthalen-1- yl)phenyl)amino)azetidine-1- carbonyl)cyclohexyl)-6,7- dihydropyrrolo[3,4-f]isoindole- 1,3(2H,5H)-dione |
| E 387 | 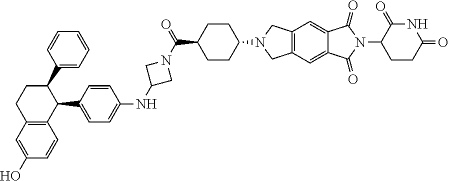 [SMILES: O=C1CCC(N2C(=O)c3cc4c(cc3C2=O)CN([C@H]2CC[C@H](C(=O)N3CC(Nc5ccc([C@H]6c7ccc(O)cc7CC[C@H]6c6ccccc6)cc5)C3)CC2)C4)C(=O)N1] | 2-(2,6-dioxopiperidin-3-yl)-6-((1S,4r)-4- (3-((4-((1S,2R)-6-hydroxy-2-phenyl- 1,2,3,4-tetrahydronaphthalen-1- yl)phenyl)amino)azetidine-1- carbonyl)cyclohexyl)-6,7- dihydropyrrolo[3,4-f]isoindole- 1,3(2H,5H)-dione |
| E 388 | 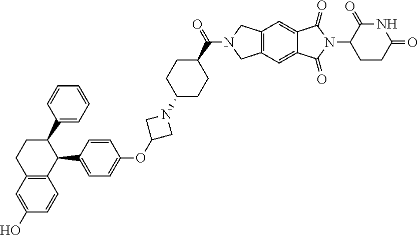 [SMILES: O=C1CCC(N2C(=O)c3cc4c(cc3C2=O)CN(C(=O)[C@H]2CC[C@H](N3CC(Oc5ccc([C@H]6c7ccc(O)cc7CC[C@H]6c6ccccc6)cc5)C3)CC2)C4)C(=O)N1] | 2-(2,6-dioxopiperidin-3-yl)-6-((1S,4r)-4- (3-(4-((1S,2R)-6-hydroxy-2-phenyl- 1,2,3,4-tetrahydronaphthalen-1- yl)phenoxy)azetidin-1-yl)cyclohexane- 1-carbonyl)-6,7-dihydropyrrolo[3,4- f]isoindole-1,3(2H,5H)-dione |
| E 389 | 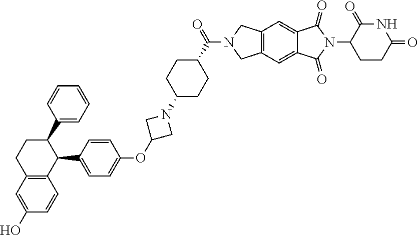 [SMILES: O=C1CCC(N2C(=O)c3cc4c(cc3C2=O)CN(C(=O)[C@H]2CC[C@@H](N3CC(Oc5ccc([C@H]6c7ccc(O)cc7CC[C@H]6c6ccccc6)cc5)C3)CC2)C4)C(=O)N1] | 2-(2,6-dioxopiperidin-3-yl)-6-((1R,4s)- 4-(3-(4-((1S,2R)-6-hydroxy-2-phenyl- 1,2,3,4-tetrahydronaphthalen-1- yl)phenoxy)azetidin-1-yl)cyclohexane- 1-carbonyl)-6,7-dihydropyrrolo[3,4- f]isoindole-1,3(2H,5H)-dione |
| E 390 | 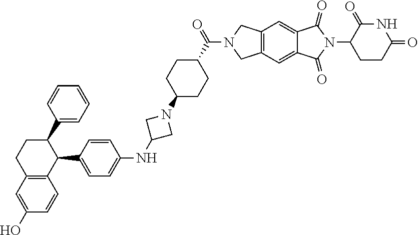 [SMILES: O=C1CCC(N2C(=O)c3cc4c(cc3C2=O)CN(C(=O)[C@H]2CC[C@H](N3CC(Nc5ccc([C@H]6c7ccc(O)cc7CC[C@H]6c6ccccc6)cc5)C3)CC2)C4)C(=O)N1] | 2-(2,6-dioxopiperidin-3-yl)-6-((1S,4r)-4- (3-((4-((1S,2R)-6-hydroxy-2-phenyl- 1,2,3,4-tetrahydronaphthalen-1- yl)phenyl)amino)azetidin-1- yl)cyclohexane-1-carbonyl)-6,7- dihydropyrrolo[3,4-f]isoindole- 1,3(2H,5H)-dione |
| E 391 | 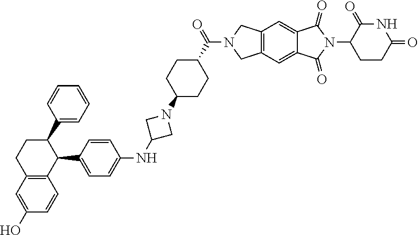 [SMILES: O=C1CCC(N2C(=O)c3cc4c(cc3C2=O)CN(C(=O)[C@H]2CC[C@H](N3CC(Nc5ccc([C@H]6c7ccc(O)cc7CC[C@H]6c6ccccc6)cc5)C3)CC2)C4)C(=O)N1] | 2-(2,6-dioxopiperidin-3-yl)-6-((1S,4r)-4- (3-((4-((1S,2R)-6-hydroxy-2-phenyl- 1,2,3,4-tetrahydronaphthalen-1- yl)phenyl)amino)azetidin-1- yl)cyclohexane-1-carbonyl)-6,7- dihydropyrrolo[3,4-f]isoindole- 1,3(2H,5H)-dione |
| E 392 | 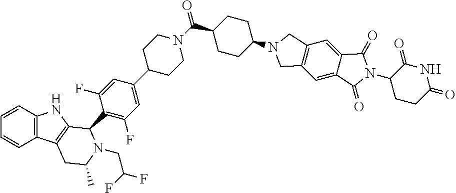 [SMILES: C[C@@H]1Cc2c([nH]c3ccccc23)[C@@H](c2c(F)cc(C3CCN(C(=O)[C@H]4CC[C@@H](N5Cc6cc7c(cc6C5)C(=O)N(C5CCC(=O)NC5=O)C7=O)CC4)CC3)cc2F)N1CC(F)F] | 6-((1S,4s)-4-(4-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| E 393 | 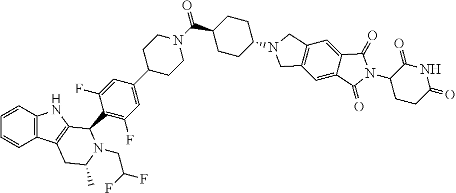 [SMILES: C[C@@H]1Cc2c([nH]c3ccccc23)[C@@H](c2c(F)cc(C3CCN(C(=O)[C@H]4CC[C@H](N5Cc6cc7c(cc6C5)C(=O)N(C5CCC(=O)NC5=O)C7=O)CC4)CC3)cc2F)N1CC(F)F] | 6-((1R,4r)-4-(4-(4-((1R,3R)-2-(2,2- difluoroethyl)-3-methyl-2,3,4,9- tetrahydro-1H-pyrido[3,4-b]indol-1-yl)- 3,5-difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| E 394 | 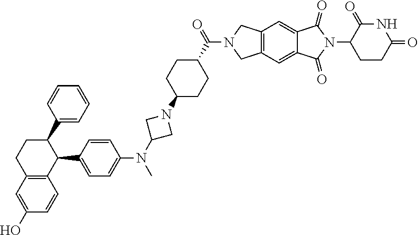 [SMILES: CN(c1ccc([C@H]2c3ccc(O)cc3CC[C@H]2c2ccccc2)cc1)C1CN([C@H]2CC[C@H](C(=O)N3Cc4cc5c(cc4C3)C(=O)N(C3CCC(=O)NC3=O)C5=O)CC2)C1] | 2-(2,6-dioxopiperidin-3-yl)-6-((1S,4r)-4- (3-((4-((1S,2R)-6-hydroxy-2-phenyl- 1,2,3,4-tetrahydronaphthalen-1- yl)phenyl)(methyl)amino)azetidin-1- yl)cyclohexane-1-carbonyl)-6,7- dihydropyrrolo[3,4-f]isoindole- 1,3(2H,5H)-dione |
| E 395 | 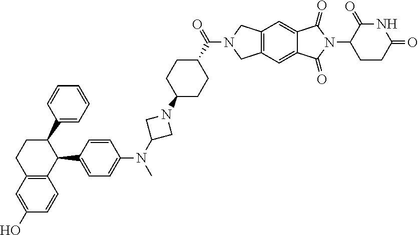 [SMILES: CN(c1ccc([C@H]2c3ccc(O)cc3CC[C@H]2c2ccccc2)cc1)C1CN([C@H]2CC[C@H](C(=O)N3Cc4cc5c(cc4C3)C(=O)N(C3CCC(=O)NC3=O)C5=O)CC2)C1] | 2-(2,6-dioxopiperidin-3-yl)-6-((1R,4s)- 4-(3-((4-((1S,2R)-6-hydroxy-2-phenyl- 1,2,3,4-tetrahydronaphthalen-1- yl)phenyl)(methyl)amino)azetidin-1- yl)cyclohexane-1-carbonyl)-6,7- dihydropyrrolo[3,4-f]isoindole- 1,3(2H,5H)-dione |
| E 396 | 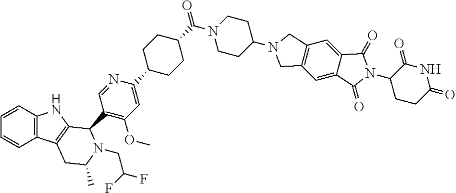 [SMILES: COc1cc([C@H]2CC[C@@H](C(=O)N3CCC(N4Cc5cc6c(cc5C4)C(=O)N(C4CCC(=O)NC4=O)C6=O)CC3)CC2)ncc1[C@@H]1c2[nH]c3ccccc3c2C[C@@H](C)N1CC(F)F] | 6-(1-((1S,4s)-4-(5-((1R,3R)-2-(2,2- difluoroethyl)-3-methyl-2,3,4,9- tetrahydro-1H-pyrido[3,4-b]indol-1-yl)- 4-methoxypyridin-2-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 397 | 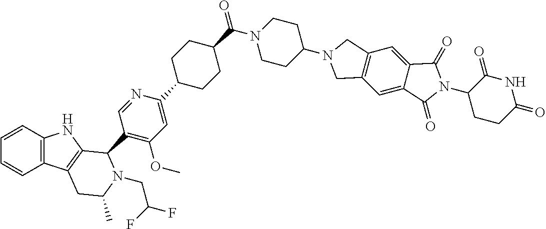 [SMILES: COc1cc([C@H]2CC[C@H](C(=O)N3CCC(N4Cc5cc6c(cc5C4)C(=O)N(C4CCC(=O)NC4=O)C6=O)CC3)CC2)ncc1[C@@H]1c2[nH]c3ccccc3c2C[C@@H](C)N1CC(F)F] | 6-(1-((1R,4r)-4-(5-((1R,3R)-2-(2,2- difluoroethyl)-3-methyl-2,3,4,9- tetrahydro-1H-pyrido[3,4-b]indol-1-yl)- 4-methoxypyridin-2-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 398 | 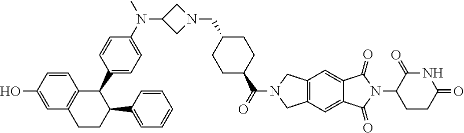 [SMILES: CN(c1ccc([C@@H]2c3ccc(O)cc3CC[C@@H]2c2ccccc2)cc1)C1CN(C[C@H]2CC[C@H](C(=O)N3Cc4cc5c(cc4C3)C(=O)N(C3CCC(=O)NC3=O)C5=O)CC2)C1] | 2-(2,6-dioxopiperidin-3-yl)-6-((1R,4r)- 4-((3-((4-((1R,2S)-6-hydroxy-2-phenyl- 1,2,3,4-tetrahydronaphthalen-1- yl)phenyl)(methyl)amino)azetidin-1- yl)methyl)cyclohexane-1-carbonyl)-6,7- dihydropyrrolo[3,4-f]isoindole- 1,3(2H,5H)-dione |
| E 399 | 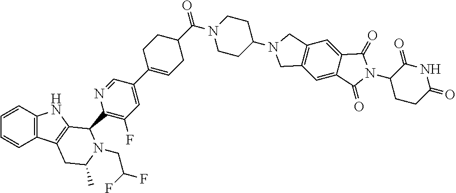 [SMILES: C[C@@H]1Cc2c([nH]c3ccccc23)[C@@H](c2ncc(C3=CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4-(6-((1S,3R)-2-(2,2- difluoroethyl)-3-methyl-2,3,4,9- tetrahydro-1H-pyrido[3,4-b]indol-1-yl)- 5-fluoropyridin-3-yl)cyclohex-3-ene-1- carbonyl)piperidin-4-yl)-2-(2,6- dioxopiperidin-3-yl)-6,7- dihydropyrrolo[3,4-f]isoindole- 1,3(2H,5H)-dione |
| E 400 | 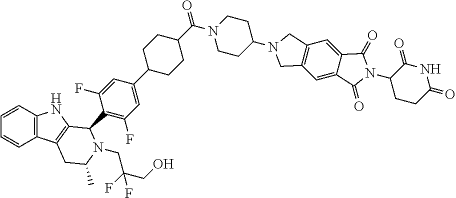 [SMILES: C[C@@H]1Cc2c([nH]c3ccccc23)[C@@H](c2c(F)cc(C3CCC(C(=O)N4CCC(N5Cc6cc7c(cc6C5)C(=O)N(C5CCC(=O)NC5=O)C7=O)CC4)CC3)cc2F)N1CC(F)(F)CO] | 6-(1-(4-(4-((1R,3R)-2-(2,2-difluoro-3- hydroxypropyl)-3-methyl-2,3,4,9- tetrahydro-1H-pyrido[3,4-b]indol-1-yl)- 3,5-difluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 402 | 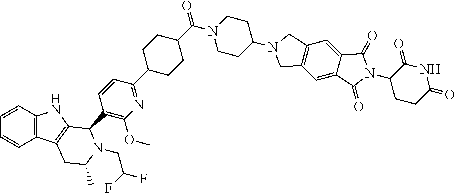 [SMILES: COc1nc(C2CCC(C(=O)N3CCC(N4Cc5cc6c(cc5C4)C(=O)N(C4CCC(=O)NC4=O)C6=O)CC3)CC2)ccc1[C@@H]1c2[nH]c3ccccc3c2C[C@@H](C)N1CC(F)F] | 6-(1-(4-(5-((1R,3R)-2-(2,2- difluoroethyl)-3-methyl-2,3,4,9- tetrahydro-1H-pyrido[3,4-b]indol-1-yl)- 6-methoxypyridin-2-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 403 | 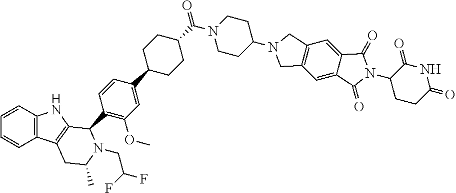 [SMILES: COc1cc([C@H]2CC[C@H](C(=O)N3CCC(N4Cc5cc6c(cc5C4)C(=O)N(C4CCC(=O)NC4=O)C6=O)CC3)CC2)ccc1[C@@H]1c2[nH]c3ccccc3c2C[C@@H](C)N1CC(F)F] | 6-(1-((1R,4r)-4-(4-((1R,3R)-2-(2,2- difluoroethyl)-3-methyl-2,3,4,9- tetrahydro-1H-pyrido[3,4-b]indol-1-yl)- 3-methoxyphenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 404 | 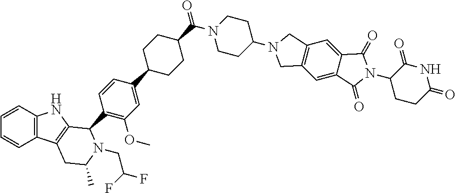 [SMILES: COc1cc([C@H]2CC[C@@H](C(=O)N3CCC(N4Cc5cc6c(cc5C4)C(=O)N(C4CCC(=O)NC4=O)C6=O)CC3)CC2)ccc1[C@@H]1c2[nH]c3ccccc3c2C[C@@H](C)N1CC(F)F] | 6-(1-((1S,4s)-4-(4-((1R,3R)-2-(2,2- difluoroethyl)-3-methyl-2,3,4,9- tetrahydro-1H-pyrido[3,4-b]indol-1-yl)- 3-methoxyphenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 405 | 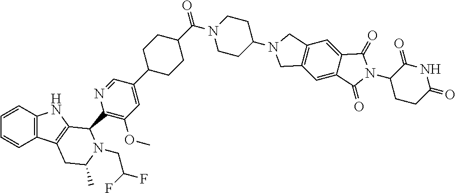 [SMILES: COc1cc(C2CCC(C(=O)N3CCC(N4Cc5cc6c(cc5C4)C(=O)N(C4CCC(=O)NC4=O)C6=O)CC3)CC2)cnc1[C@@H]1c2[nH]c3ccccc3c2C[C@@H](C)N1CC(F)F] | 6-(1-(4-(6-((1S,3R)-2-(2,2- difluoroethyl)-3-methyl-2,3,4,9- tetrahydro-1H-pyrido[3,4-b]indol-1-yl)- 5-methoxypyridin-3-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 406 | 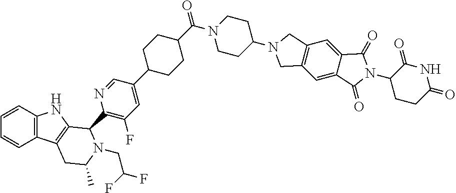 [SMILES: C[C@@H]1Cc2c([nH]c3ccccc23)[C@@H](c2ncc(C3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4-(6-((1S,3R)-2-(2,2- difluoroethyl)-3-methyl-2,3,4,9- tetrahydro-1H-pyrido[3,4-b]indol-1-yl)- 5-fluoropyridin-3-yl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 407 | 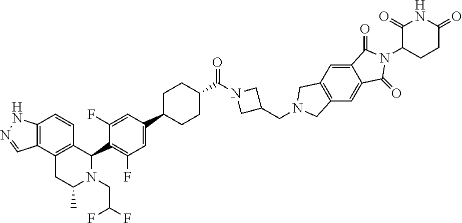 [SMILES: C[C@@H]1Cc2c(ccc3[nH]ncc23)[C@@H](c2c(F)cc([C@H]3CC[C@H](C(=O)N4CC(CN5Cc6cc7c(cc6C5)C(=O)N(C5CCC(=O)NC5=O)C7=O)C4)CC3)cc2F)N1CC(F)F] | 6-((1-((1S,4r)-4-(4-((6S,8R)-7-(2,2- difluoroethyl)-8-methyl-6,7,8,9- tetrahydro-3H-pyrazolo[4,3- flisoquinolin-6-yl)-3,5- difluorophenyl)cyclohexane-1- carbonyl)azetidin-3-yl)methyl)-2-(2,6- dioxopiperidin-3-yl)-6,7- dihydropyrrolo[3,4-f]isoindole- 1,3(2H,5H)-dione |
| E 408 | 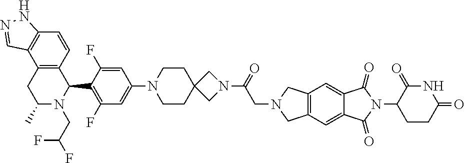 [SMILES: C[C@@H]1Cc2c(ccc3[nH]ncc23)[C@@H](c2c(F)cc(N3CCC4(CC3)CN(C(=O)CN3Cc5cc6c(cc5C3)C(=O)N(C3CCC(=O)NC3=O)C6=O)C4)cc2F)N1CC(F)F] | 6-(2-(7-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 2,7-diazaspiro[3.5]nonan-2-yl)-2- oxoethyl)-2-(2,6-dioxopiperidin-3-yl)- 6,7-dihydropyrrolo[3,4-f]isoindole- 1,3(2H,5H)-dione |
| E 409 | 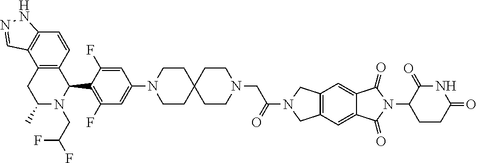 [SMILES: C[C@@H]1Cc2c(ccc3[nH]ncc23)[C@@H](c2c(F)cc(N3CCC4(CCN(CC(=O)N5Cc6cc7c(cc6C5)C(=O)N(C5CCC(=O)NC5=O)C7=O)CC4)CC3)cc2F)N1CC(F)F] | 6-(2-(9-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 3,9-diazaspiro[5.5]undecan-3-yl)acetyl)- 2-(2,6-dioxopiperidin-3-yl)-6,7- dihydropyrrolo[3,4-f]isoindole- 1,3(2H,5H)-dione |
| E 410 | 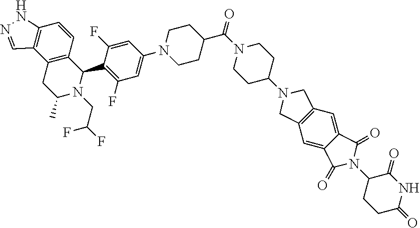 [SMILES: C[C@@H]1Cc2c(ccc3[nH]ncc23)[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 411 | 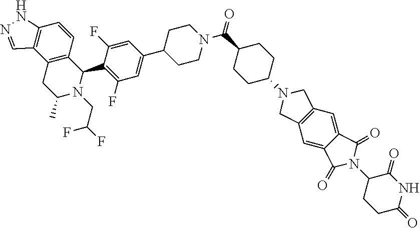 [SMILES: C[C@@H]1Cc2c(ccc3[nH]ncc23)[C@@H](c2c(F)cc(C3CCN(C(=O)[C@H]4CC[C@H](N5Cc6cc7c(cc6C5)C(=O)N(C5CCC(=O)NC5=O)C7=O)CC4)CC3)cc2F)N1CC(F)F] | 6-((1S,4r)-4-(4-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| E 412 | 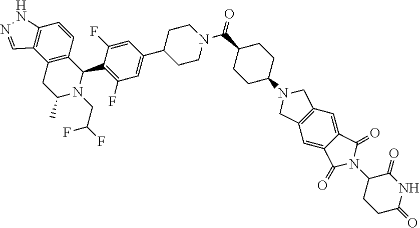 [SMILES: C[C@@H]1Cc2c(ccc3[nH]ncc23)[C@@H](c2c(F)cc(C3CCN(C(=O)[C@H]4CC[C@@H](N5Cc6cc7c(cc6C5)C(=O)N(C5CCC(=O)NC5=O)C7=O)CC4)CC3)cc2F)N1CC(F)F] | 6-((1R,4s)-4-(4-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| E 413 | 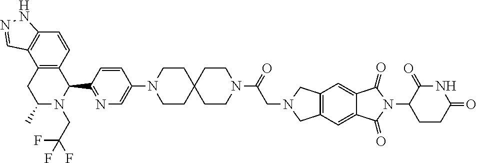 [SMILES: C[C@@H]1Cc2c(ccc3[nH]ncc23)[C@@H](c2ccc(N3CCC4(CCN(C(=O)CN5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(2-(9-(6- ((6S,8R)-8-methyl-7-(2,2,2- trifluoroethyl)-6,7,8,9-tetrahydro-3H- pyrazolo[4,3-f]isoquinolin-6-yl)pyridin- 3-yl)-3,9-diazaspiro[5.5]undecan-3-yl)- 2-oxoethyl)-6,7-dihydropyrrolo[3,4- f]isoindole-1,3(2H,5H)-dione |
| E 414 | 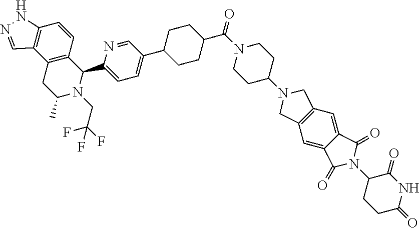 [SMILES: C[C@@H]1Cc2c(ccc3[nH]ncc23)[C@@H](c2ccc(C3CCC(C(=O)N4CCC(N5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(1-(4-(6- ((6S,8R)-8-methyl-7-(2,2,2- trifluoroethyl)-6,7,8,9-tetrahydro-3H- pyrazolo[4,3-f]isoquinolin-6-yl)pyridin- 3-yl)cyclohexane-1-carbonyl)piperidin- 4-yl)-6,7-dihydropyrrolo[3,4- f]isoindole-1,3(2H,5H)-dione |
| E 415 | 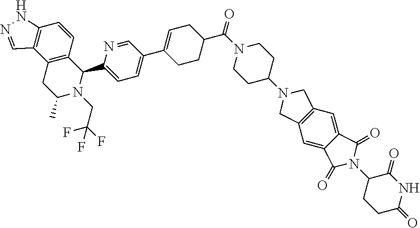 [SMILES: C[C@@H]1Cc2c(ccc3[nH]ncc23)[C@@H](c2ccc(C3=CCC(C(=O)N4CCC(N5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(1-(4-(6- ((6S,8R)-8-methyl-7-(2,2,2- trifluoroethyl)-6,7,8,9-tetrahydro-3H- pyrazolo[4,3-f]isoquinolin-6-yl)pyridin- 3-yl)cyclohex-3-ene-1- carbonyl)piperidin-4-yl)-6,7- dihydropyrrolo[3,4-f]isoindole- 1,3(2H,5H)-dione |
| E 416 | 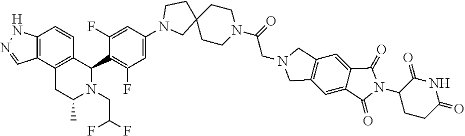 [SMILES: C[C@@H]1Cc2c(ccc3[nH]ncc23)[C@@H](c2c(F)cc(N3CCC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 2,8-diazaspiro[4.5]decan-8-yl)-2- oxoethyl)-2-(2,6-dioxopiperidin-3-yl)- 6,7-dihydropyrrolo[3,4-f]isoindole- 1,3(2H,5H)-dione |
| E 417 | 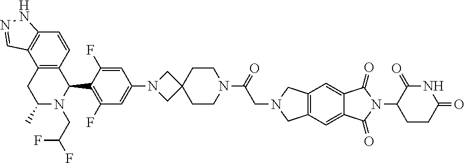 [SMILES: C[C@@H]1Cc2c(ccc3[nH]ncc23)[C@@H](c2c(F)cc(N3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 2,7-diazaspiro[3.5]nonan-7-yl)-2- oxoethyl)-2-(2,6-dioxopiperidin-3-yl)- 6,7-dihydropyrrolo[3,4-f]isoindole- 1,3(2H,5H)-dione |
| E 418 | 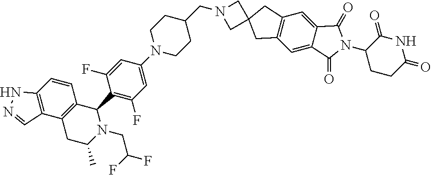 [SMILES: C[C@@H]1Cc2c(ccc3[nH]ncc23)[C@@H](c2c(F)cc(N3CCC(CN4CC5(Cc6cc7c(cc6C5)C(=O)N(C5CCC(=O)NC5=O)C7=O)C4)CC3)cc2F)N1CC(F)F] | 1-((1-(4-((6S,8R)-7-(2,2-difluoroethyl)- 8-methyl-6,7,8,9-tetrahydro-3H- pyrazolo[4,3-f]isoquinolin-6-yl)-3,5- difluorophenyl)piperidin-4-yl)methyl)- 2′-(2,6-dioxopiperidin-3-yl)-5′,7′- dihydro-1′H-spiro[azetidine-3,6′- cyclopenta[f]isoindole]-1′,3′(2′H)-dione |
| E 419 | 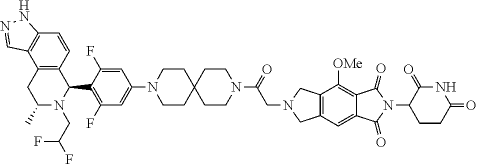 [SMILES: COc1c2c(cc3c1C(=O)N(C1CCC(=O)NC1=O)C3=O)CN(CC(=O)N1CCC3(CC1)CCN(c1cc(F)c([C@@H]4c5ccc6[nH]ncc6c5C[C@@H](C)N4CC(F)F)c(F)c1)CC3)C2] | 6-(2-(9-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 3,9-diazaspiro[5.5]undecan-3-yl)-2- oxoethyl)-2-(2,6-dioxopiperidin-3-yl)-4- methoxy-6,7-dihydropyrrolo[3,4 f]isoindole-1,3(2H,5H)-dione |
| E 420 | 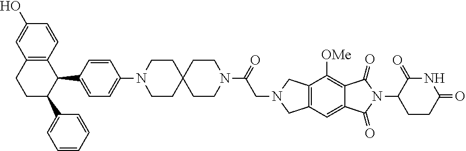 [SMILES: COc1c2c(cc3c1C(=O)N(C1CCC(=O)NC1=O)C3=O)CN(CC(=O)N1CCC3(CC1)CCN(c1ccc([C@@H]4c5ccc(O)cc5CC[C@@H]4c4ccccc4)cc1)CC3)C2] | 2-(2,6-dioxopiperidin-3-yl)-6-(2-(9-(4- ((1R,2S)-6-hydroxy-2-phenyl-1,2,3,4- tetrahydronaphthalen-1-yl)phenyl)-3,9- diazaspiro[5.5]undecan-3-yl)-2- oxoethyl)-4-methoxy-6,7- dihydropyrrolo[3,4-f]isoindole- 1,3(2H,5H)-dione |
| E 421 | 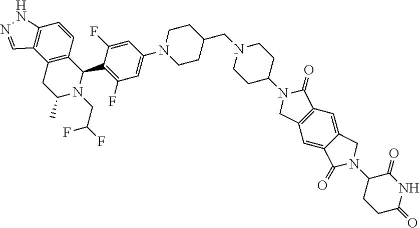 [SMILES: C[C@@H]1Cc2c(ccc3[nH]ncc23)[C@@H](c2c(F)cc(N3CCC(CN4CCC(N5Cc6cc7c(cc6C5=O)CN(C5CCC(=O)NC5=O)C7=O)CC4)CC3)cc2F)N1CC(F)F] | 2-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperidin-4-yl)-6-(2,6- dioxopiperidin-3-yl)-2,3,6,7- tetrahydropyrrolo[3,4-f]isoindole-1,5- dione |
| E 422 | 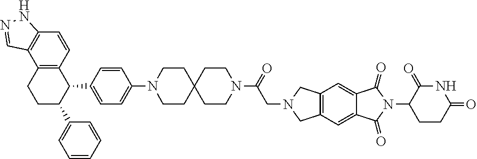 [SMILES: O=C1CCC(N2C(=O)c3cc4c(cc3C2=O)CN(CC(=O)N2CCC3(CC2)CCN(c2ccc([C@H]5c6ccc7[nH]ncc7c6CC[C@H]5c5ccccc5)cc2)CC3)C4)C(=O)N1] | 2-(2,6-dioxopiperidin-3-yl)-6-(2-oxo-2- (9-(4-((6S,7R)-7-phenyl-6,7,8,9- tetrahydro-3H-benzo[e]indazol-6- yl)phenyl)-3,9-diazaspiro[5.5]undecan- 3-yl)ethyl)-6,7-dihydropyrrolo[3,4- f]isoindole-1,3(2H,5H)-dione |
| E 423 | 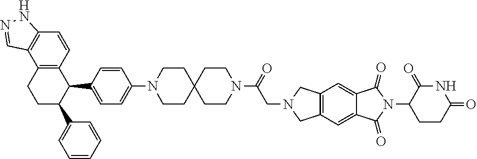 [SMILES: O=C1CCC(N2C(=O)c3cc4c(cc3C2=O)CN(CC(=O)N2CCC3(CC2)CCN(c2ccc([C@@H]5c6ccc7[nH]ncc7c6CC[C@@H]5c5ccccc5)cc2)CC3)C4)C(=O)N1] | 2-(2,6-dioxopiperidin-3-yl)-6-(2-oxo-2- (9-(4-((6R,7S)-7-phenyl-6,7,8,9- tetrahydro-3H-benzo[e]indazol-6- yl)phenyl)-3,9-diazaspiro[5.5]undecan- 3-yl)ethyl)-6,7-dihydropyrrolo[3,4- f]isoindole-1,3(2H,5H)-dione |
| E 424 | 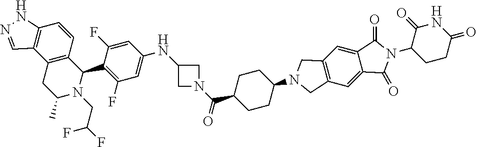 [SMILES: C[C@@H]1Cc2c(ccc3[nH]ncc23)[C@@H](c2c(F)cc(NC3CN(C(=O)[C@H]4CC[C@@H](N5Cc6cc7c(cc6C5)C(=O)N(C5CCC(=O)NC5=O)C7=O)CC4)C3)cc2F)N1CC(F)F] | 6-((1R,4s)-4-(3-((4-((6S,8R)-7-(2,2- difluoroethyl)-8-methyl-6,7,8,9- tetrahydro-3H-pyrazolo[4,3- flisoquinolin-6-yl)-3,5- difluorophenyl)amino)azetidine-1- carbonyl)cyclohexyl)-2-(2,6- dioxopiperidin-3-yl)-6,7- dihydropyrrolo[3,4-f]isoindole- 1,3(2H,5H)-dione |
| E 425 | 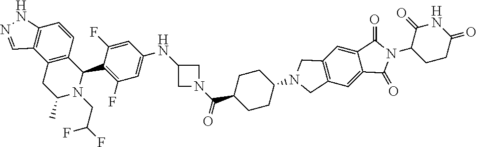 [SMILES: C[C@@H]1Cc2c(ccc3[nH]ncc23)[C@@H](c2c(F)cc(NC3CN(C(=O)[C@H]4CC[C@H](N5Cc6cc7c(cc6C5)C(=O)N(C5CCC(=O)NC5=O)C7=O)CC4)C3)cc2F)N1CC(F)F] | 6-((1S,4r)-4-(3-((4-((6S,8R)-7-(2,2- difluoroethyl)-8-methyl-6,7,8,9- tetrahydro-3H-pyrazolo[4,3- flisoquinolin-6-yl)-3,5- difluorophenyl)amino)azetidine-1- carbonyl)cyclohexyl)-2-(2,6- dioxopiperidin-3-yl)-6,7- dihydropyrrolo[3,4-f]isoindole- 1,3(2H,5H)-dione |
| E 426 | 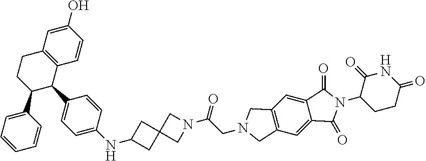 [SMILES: O=C1CCC(N2C(=O)c3cc4c(cc3C2=O)CN(CC(=O)N2CC3(CC(Nc5ccc([C@@H]6c7ccc(O)cc7CC[C@@H]6c6ccccc6)cc5)C3)C2)C4)C(=O)N1] | 2-(2,6-dioxopiperidin-3-yl)-6-(2-(6-((4- ((1R,2S)-6-hydroxy-2-phenyl-1,2,3,4- tetrahydronaphthalen-1- yl)phenyl)amino)-2- azaspiro[3.3]heptan-2-yl)-2-oxoethyl)- 6,7-dihydropyrrolo[3,4-f]isoindole- 1,3(2H,5H)-dione |
| E 427 | 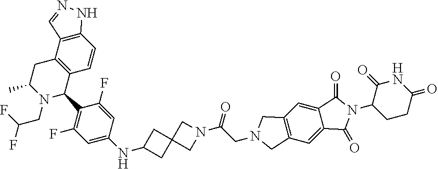 [SMILES: C[C@@H]1Cc2c(ccc3[nH]ncc23)[C@@H](c2c(F)cc(NC3CC4(C3)CN(C(=O)CN3Cc5cc6c(cc5C3)C(=O)N(C3CCC(=O)NC3=O)C6=O)C4)cc2F)N1CC(F)F] | 6-(2-(6-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)-2- azaspiro[3.3]heptan-2-yl)-2-oxoethyl)-2- (2,6-dioxopiperidin-3-yl)-6,7- dihydropyrrolo[3,4-f]isoindole- 1,3(2H,5H)-dione |
| E 428 | 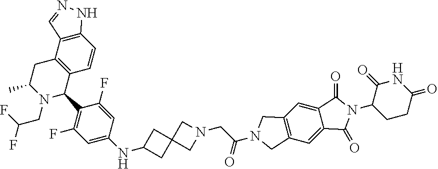 [SMILES: C[C@@H]1Cc2c(ccc3[nH]ncc23)[C@@H](c2c(F)cc(NC3CC4(C3)CN(CC(=O)N3Cc5cc6c(cc5C3)C(=O)N(C3CCC(=O)NC3=O)C6=O)C4)cc2F)N1CC(F)F] | 6-(2-(6-((4-((6S,8R)-7-(2,2- difluoroethyl)-8-methyl-6,7,8,9- tetrahydro-3H-pyrazolo[4,3- flisoquinolin-6-yl)-3,5- difluorophenyl)amino)-2- azaspiro[3.3]heptan-2-yl)acetyl)-2-(2,6- dioxopiperidin-3-yl)-6,7- dihydropyrrolo[3,4-f]isoindole- 1,3(2H,5H)-dione |
| E 429 | 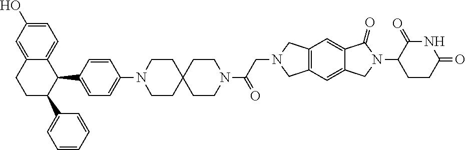 [SMILES: O=C1CCC(N2Cc3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | 3-(6-(2-(9-(4-((1R,2S)-6-hydroxy-2- phenyl-1,2,3,4-tetrahydronaphthalen-1- yl)phenyl)-3,9-diazaspiro[5.5]undecan- 3-yl)-2-oxoethyl)-1-oxo-3,5,6,7- tetrahydropyrrolo[3,4-f]isoindol-2(1H)- yl)piperidine-2,6-dione |
| E 430 | 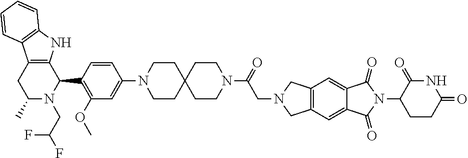 [SMILES: COc1cc(N2CCC3(CCN(C(=O)CN4Cc5cc6c(cc5C4)C(=O)N(C4CCC(=O)NC4=O)C6=O)CC3)CC2)ccc1[C@@H]1c2[nH]c3ccccc3c2C[C@@H](C)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3-methoxyphenyl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 431 | 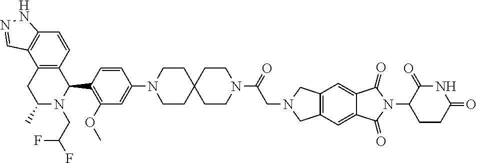 [SMILES: COc1cc(N2CCC3(CCN(C(=O)CN4Cc5cc6c(cc5C4)C(=O)N(C4CCC(=O)NC4=O)C6=O)CC3)CC2)ccc1[C@@H]1c2ccc3[nH]ncc3c2C[C@@H](C)N1CC(F)F] | 6-(2-(9-(4-((6S,8R)-7-(2,2- difluoroethyl)-8-methyl-6,7,8,9- tetrahydro-3H-pyrazolo[4,3- flisoquinolin-6-yl)-3-methoxyphenyl)- 3,9-diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 432 | 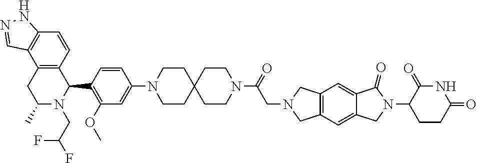 [SMILES: COc1cc(N2CCC3(CCN(C(=O)CN4Cc5cc6c(cc5C4)C(=O)N(C4CCC(=O)NC4=O)C6)CC3)CC2)ccc1[C@@H]1c2ccc3[nH]ncc3c2C[C@@H](C)N1CC(F)F] | 3-(6-(2-(9-(4-((6S,8R)-7-(2,2- difluoroethyl)-8-methyl-6,7,8,9- tetrahydro-3H-pyrazolo[4,3- flisoquinolin-6-yl)-3-methoxyphenyl)- 3,9-diazaspiro[5.5]undecan-3-yl)-2- oxoethyl)-1-oxo-3,5,6,7- tetrahydropyrrolo[3,4-f]isoindol-2(1H)- yl)piperidine-2,6-dione |
| E 433 | 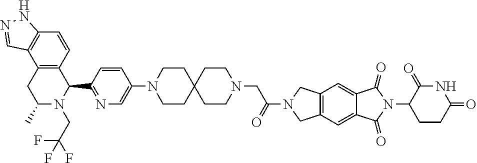 [SMILES: C[C@@H]1Cc2c(ccc3[nH]ncc23)[C@@H](c2ccc(N3CCC4(CCN(CC(=O)N5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(2-(9-(6- ((6S,8R)-8-methyl-7-(2,2,2- trifluoroethyl)-6, 7,8,9-tetrahydro-3H- pyrazolo[4,3-f]isoquinolin-6-yl)pyridin- 3-yl)-3,9-diazaspiro[5.5]undecan-3- yl)acetyl)-6,7-dihydropyrrolo[3,4- f]isoindole-1,3(2H,5H)-dione |
| E 434 | 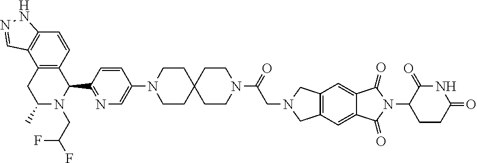 [SMILES: C[C@@H]1Cc2c(ccc3[nH]ncc23)[C@@H](c2ccc(N3CCC4(CCN(C(=O)CN5Cc6cc7c(cc6C5)C(=O)N(C5CCC(=O)NC5=O)C7=O)CC4)CC3)cn2)N1CC(F)F] | 6-(2-(9-(6-((6S,8R)-7-(2,2- difluoroethyl)-8-methyl-6,7,8,9- tetrahydro-3H-pyrazolo[4,3- flisoquinolin-6-yl)pyridin-3-yl)-3,9- diazaspiro[5.5]undecan-3-yl)-2- oxoethyl)-2-(2,6-dioxopiperidin-3-yl)- 6,7-dihydropyrrolo[3,4-f]isoindole- 1,3(2H,5H)-dione |
| E 435 | 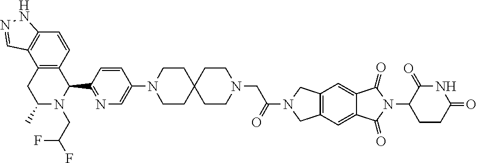 [SMILES: C[C@@H]1Cc2c(ccc3[nH]ncc23)[C@@H](c2ccc(N3CCC4(CCN(CC(=O)N5Cc6cc7c(cc6C5)C(=O)N(C5CCC(=O)NC5=O)C7=O)CC4)CC3)cn2)N1CC(F)F] | 6-(2-(9-(6-((6S,8R)-7-(2,2- difluoroethyl)-8-methyl-6,7,8,9- tetrahydro-3H-pyrazolo[4,3- flisoquinolin-6-yl)pyridin-3-yl)-3,9- diazaspiro[5.5]undecan-3-yl)acetyl)-2- (2,6-dioxopiperidin-3-yl)-6,7- dihydropyrrolo[3,4-f]isoindole- 1,3(2H,5H)-dione |
| E 436 | 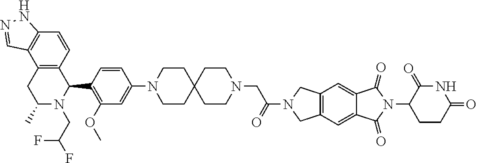 [SMILES: COc1cc(N2CCC3(CCN(CC(=O)N4Cc5cc6c(cc5C4)C(=O)N(C4CCC(=O)NC4=O)C6=O)CC3)CC2)ccc1[C@@H]1c2ccc3[nH]ncc3c2C[C@@H](C)N1CC(F)F] | 6-(2-(9-(4-((6S,8R)-7-(2,2- difluoroethyl)-8-methyl-6,7,8,9- tetrahydro-3H-pyrazolo[4,3- flisoquinolin-6-yl)-3-methoxyphenyl)- 3,9-diazaspiro[5.5]undecan-3-yl)acetyl)- 2-(2,6-dioxopiperidin-3-yl)-6,7- dihydropyrrolo[3,4-f]isoindole- 1,3(2H,5H)-dione |
| E 437 | 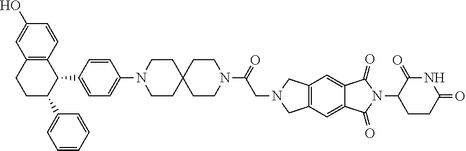 [SMILES: O=C1CCC(N2C(=O)c3cc4c(cc3C2=O)CN(CC(=O)N2CCC3(CC2)CCN(c2ccc([C@H]5c6ccc(O)cc6CC[C@H]5c5ccccc5)cc2)CC3)C4)C(=O)N1] | 2-(2,6-dioxopiperidin-3-yl)-6-(2-(9-(4- ((1S,2R)-6-hydroxy-2-phenyl-1,2,3,4- tetrahydronaphthalen-1-yl)phenyl)-3,9- diazaspiro[5.5]undecan-3-yl)-2- oxoethyl)-6,7-dihydropyrrolo[3,4- f]isoindole-1,3(2H,5H)-dione |
| E 438 | 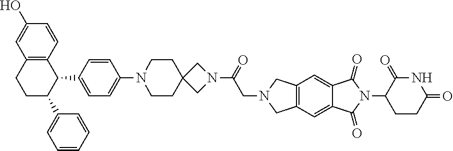 [SMILES: O=C1CCC(N2C(=O)c3cc4c(cc3C2=O)CN(CC(=O)N2CC3(CCN(c5ccc([C@H]6c7ccc(O)cc7CC[C@H]6c6ccccc6)cc5)CC3)C2)C4)C(=O)N1] | 2-(2,6-dioxopiperidin-3-yl)-6-(2-(7-(4- ((1S,2R)-6-hydroxy-2-phenyl-1,2,3,4- tetrahydronaphthalen-1-yl)phenyl)-2,7- diazaspiro[3.5]nonan-2-yl)-2-oxoethyl)- 6,7-dihydropyrrolo[3,4-f]isoindole- 1,3(2H,5H)-dione |
| E 439 | 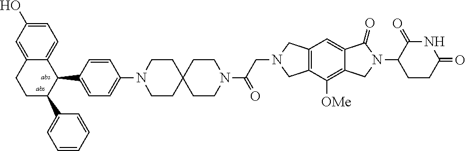 [SMILES: COc1c2c(cc3c1CN(C1CCC(=O)NC1=O)C3=O)CN(CC(=O)N1CCC3(CC1)CCN(c1ccc([C@@H]4c5ccc(O)cc5CC[C@@H]4c4ccccc4)cc1)CC3)C2] | 3-(6-(2-(9-(4-((1R,2S)-6-hydroxy-2- phenyl-1,2,3,4-tetrahydronaphthalen-1- yl)phenyl)-3,9-diazaspiro[5.5]undecan- 3-yl)-2-oxoethyl)-4-methoxy-1-oxo- 3,5,6,7-tetrahydropyrrolo[3,4-f]isoindol- 2(1H)-yl)piperidine-2,6-dione |
| E 440 | 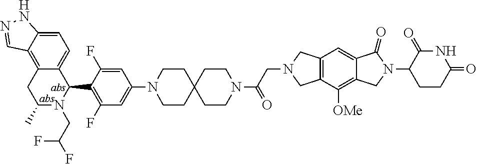 [SMILES: COc1c2c(cc3c1CN(C1CCC(=O)NC1=O)C3=O)CN(CC(=O)N1CCC3(CC1)CCN(c1cc(F)c([C@@H]4c5ccc6[nH]ncc6c5C[C@@H](C)N4CC(F)F)c(F)c1)CC3)C2] | 3-(6-(2-(9-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 3,9-diazaspiro[5.5]undecan-3-yl)-2- oxoethyl)-4-methoxy-1-oxo-3,5,6,7- tetrahydropyrrolo[3,4-f]isoindol-2(1H)- yl)piperidine-2,6-dione |
| E 441 | 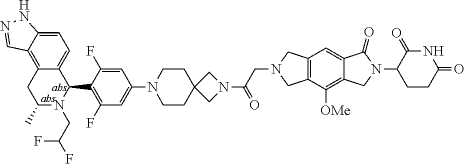 [SMILES: COc1c2c(cc3c1CN(C1CCC(=O)NC1=O)C3=O)CN(CC(=O)N1CC3(CCN(c4cc(F)c([C@@H]5c6ccc7[nH]ncc7c6C[C@@H](C)N5CC(F)F)c(F)c4)CC3)C1)C2] | 3-(6-(2-(7-(4-((6S,8R)-7-(2,2- difluoroethyl)-8-methyl-6,7,8,9- tetrahydro-3H-pyrazolo[4,3- flisoquinolin-6-yl)-3,5-difluorophenyl)- 2,7-diazaspiro[3.5]nonan-2-yl)-2- oxoethyl)-4-methoxy-1-oxo-3,5,6,7- tetrahydropyrrolo[3,4-f]isoindol-2(1H)- yl)piperidine-2,6-dione |
| E 442 | 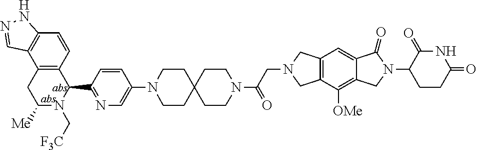 [SMILES: COc1c2c(cc3c1CN(C1CCC(=O)NC1=O)C3=O)CN(CC(=O)N1CCC3(CC1)CCN(c1ccc([C@@H]4c5ccc6[nH]ncc6c5C[C@@H](C)N4CC(F)(F)F)nc1)CC3)C2] | 3-(4-methoxy-6-(2-(9-(6-((6S,8R)-8- methyl-7-(2,2,2-trifluoroethyl)-6,7,8,9- tetrahydro-3H-pyrazolo[4,3- flisoquinolin-6-yl)pyridin-3-yl)-3,9- diazaspiro[5.5]undecan-3-yl)-2- oxoethyl)-1-oxo-3,5,6,7- tetrahydropyrrolo[3,4-f]isoindol-2(1H)- yl)piperidine-2,6-dione |
| E 443 | 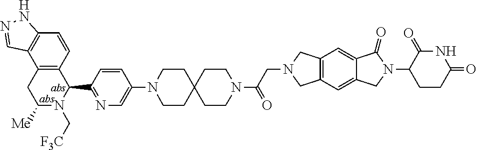 [SMILES: C[C@@H]1Cc2c(ccc3[nH]ncc23)[C@@H](c2ccc(N3CCC4(CCN(C(=O)CN5Cc6cc7c(cc6C5)C(=O)N(C5CCC(=O)NC5=O)C7)CC4)CC3)cn2)N1CC(F)(F)F] | 3-(6-(2-(9-(6-((6S,8R)-8-methyl-7- (2,2,2-trifluoroethyl)-6,7,8,9-tetrahydro- 3H-pyrazolo[4,3-f]isoquinolin-6- yl)pyridin-3-yl)-3,9- diazaspiro[5.5]undecan-3-yl)-2- oxoethyl)-1-oxo-3,5,6,7- tetrahydropyrrolo[3,4-f]isoindol-2(1H)- yl)piperidine-2,6-dione |
| E 444 | 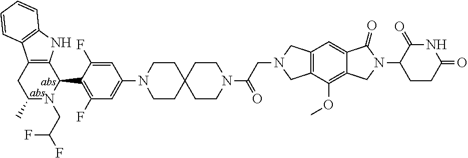 [SMILES: COc1c2c(cc3c1CN(C1CCC(=O)NC1=O)C3=O)CN(CC(=O)N1CCC3(CC1)CCN(c1cc(F)c([C@@H]4c5[nH]c6ccccc6c5C[C@@H](C)N4CC(F)F)c(F)c1)CC3)C2] | 3-(6-(2-(9-(4-((1R,3R)-2-(2,2- difluoroethyl)-3-methyl-2,3,4,9- tetrahydro-1H-pyrido[3,4-b]indol-1-yl)- 3,5-difluorophenyl)-3,9- diazaspiro[5.5]undecan-3-yl)-2- oxoethyl)-4-methoxy-1-oxo-3,5,6,7- tetrahydropyrrolo[3,4-f]isoindol-2(1H)- yl)piperidine-2,6-dione |
| E 445 | 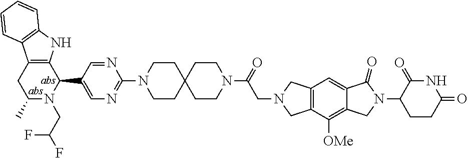 [SMILES: COc1c2c(cc3c1CN(C1CCC(=O)NC1=O)C3=O)CN(CC(=O)N1CCC3(CC1)CCN(c1ncc([C@@H]4c5[nH]c6ccccc6c5C[C@@H](C)N4CC(F)F)cn1)CC3)C2] | 3-(6-(2-(9-(5-((1R,3R)-2-(2,2- difluoroethyl)-3-methyl-2,3,4,9- tetrahydro-1H-pyrido[3,4-b]indol-1- yl)pyrimidin-2-yl)-3,9- diazaspiro[5.5]undecan-3-yl)-2- oxoethyl)-4-methoxy-1-oxo-3,5,6,7- tetrahydropyrrolo[3,4-f]isoindol-2(1H)- yl)piperidine-2,6-dione |
| E 446 | 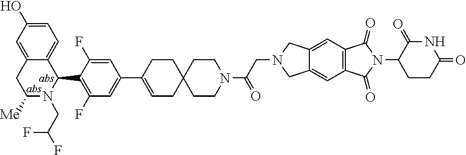 [SMILES: C[C@@H]1Cc2cc(O)ccc2[C@@H](c2c(F)cc(C3=CCC4(CC3)CCN(C(=O)CN3Cc5cc6c(cc5C3)C(=O)N(C3CCC(=O)NC3=O)C6=O)CC4)cc2F)N1CC(F)F] | 6-(2-(9-(4-((1S,3R)-2-(2,2- difluoroethyl)-6-hydroxy-3-methyl- 1,2,3,4-tetrahydroisoquinolin-1-yl)-3,5- difluorophenyl)-3-azaspiro[5.5]undec-8- en-3-yl)-2-oxoethyl)-2-(2,6- dioxopiperidin-3-yl)-6,7- dihydropyrrolo[3,4-f]isoindole- 1,3(2H,5H)-dione tetrahydroisoquinolin |
| E 447 | 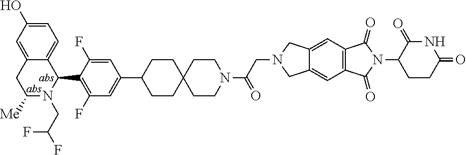 [SMILES: C[C@@H]1Cc2cc(O)ccc2[C@@H](c2c(F)cc(C3CCC4(CC3)CCN(C(=O)CN3Cc5cc6c(cc5C3)C(=O)N(C3CCC(=O)NC3=O)C6=O)CC4)cc2F)N1CC(F)F] | 6-(2-(9-(4-((1S,3R)-2-(2,2- difluoroethyl)-6-hydroxy-3-methyl- 1,2,3,4-tetrahydroisoquinolin-1-yl)-3,5- difluorophenyl)-3-azaspiro[5.5]undecan- 3-yl)-2-oxoethyl)-2-(2,6-dioxopiperidin- 3-yl)-6,7-dihydropyrrolo[3,4- f]isoindole-1,3(2H,5H)-dione |
| E 448 | 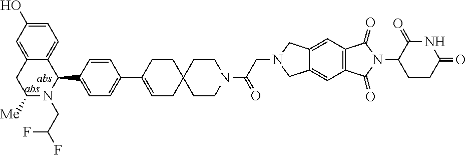 [SMILES: C[C@@H]1Cc2cc(O)ccc2[C@@H](c2ccc(C3=CCC4(CC3)CCN(C(=O)CN3Cc5cc6c(cc5C3)C(=O)N(C3CCC(=O)NC3=O)C6=O)CC4)cc2)N1CC(F)F] | 6-(2-(9-(4-((1R,3R)-2-(2,2- difluoroethyl)-6-hydroxy-3-methyl- 1,2,3,4-tetrahydroisoquinolin-1- yl)phenyl)-3-azaspiro[5.5]undec-8-en-3- yl)-2-oxoethyl)-2-(2,6-dioxopiperidin-3- yl)-6,7-dihydropyrrolo[3,4-f]isoindole- 1,3(2H,5H)-dione |
| E 449 | 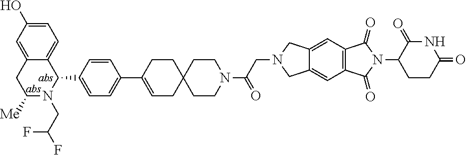 [SMILES: C[C@@H]1Cc2cc(O)ccc2[C@H](c2ccc(C3=CCC4(CC3)CCN(C(=O)CN3Cc5cc6c(cc5C3)C(=O)N(C3CCC(=O)NC3=O)C6=O)CC4)cc2)N1CC(F)F] | 6-(2-(9-(4-((1S,3R)-2-(2,2- difluoroethyl)-6-hydroxy-3-methyl- 1,2,3,4-tetrahydroisoquinolin-1- yl)phenyl)-3-azaspiro[5.5]undec-8-en-3- yl)-2-oxoethyl)-2-(2,6-dioxopiperidin-3- yl)-6, 7-dihydropyrrolo[3,4-f]isoindole- 1,3(2H,5H)-dione |
| E 450 | 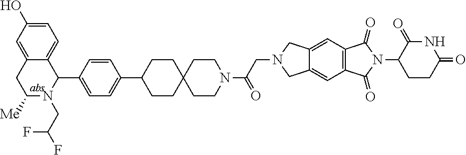 [SMILES: C[C@@H]1Cc2cc(O)ccc2C(c2ccc(C3CCC4(CC3)CCN(C(=O)CN3Cc5cc6c(cc5C3)C(=O)N(C3CCC(=O)NC3=O)C6=O)CC4)cc2)N1CC(F)F] | 6-(2-(9-(4-((3R)-2-(2,2-difluoroethyl)-6- hydroxy-3-methyl-1,2,3,4- tetrahydroisoquinolin-1-yl)phenyl)-3- azaspiro[5.5]undecan-3-yl)-2-oxoethyl)- 2-(2,6-dioxopiperidin-3-yl)-6,7- dihydropyrrolo[3,4-f]isoindole- 1,3(2H,5H)-dione |
| E 451 | 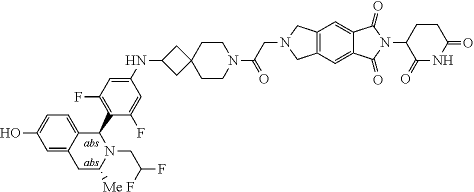 [SMILES: C[C@@H]1Cc2cc(O)ccc2[C@@H](c2c(F)cc(NC3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-((4-((1S,3R)-2-(2,2- difluoroethyl)-6-hydroxy-3-methyl- 1,2,3,4-tetrahydroisoquinolin-1-yl)-3,5- difluorophenyl)amino)-7- azaspiro[3.5]nonan-7-yl)-2-oxoethyl)-2- (2,6-dioxopiperidin-3-yl)-6,7- dihydropyrrolo[3,4-f]isoindole- 1,3(2H,5H)-dione |
| E 452 | 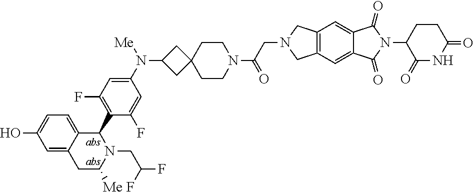 [SMILES: C[C@@H]1Cc2cc(O)ccc2[C@@H](c2c(F)cc(N(C)C3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-((4-((1S,3R)-2-(2,2- difluoroethyl)-6-hydroxy-3-methyl- 1,2,3,4-tetrahydroisoquinolin-1-yl)-3,5- difluorophenyl)(methyl)amino)-7- azaspiro[3.5]nonan-7-yl)-2-oxoethyl)-2- (2,6-dioxopiperidin-3-yl)-6,7- dihydropyrrolo[3,4-f]isoindole- 1,3(2H,5H)-dione |
| E 453 | 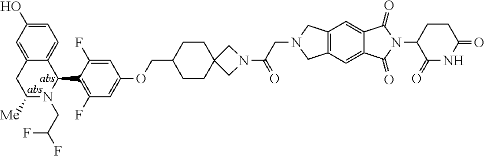 [SMILES: C[C@@H]1Cc2cc(O)ccc2[C@@H](c2c(F)cc(OCC3CCC4(CC3)CN(C(=O)CN3Cc5cc6c(cc5C3)C(=O)N(C3CCC(=O)NC3=O)C6=O)C4)cc2F)N1CC(F)F] | 6-(2-(7-((4-((1S,3R)-2-(2,2- difluoroethyl)-6-hydroxy-3-methyl- 1,2,3,4-tetrahydroisoquinolin-1-yl)-3,5- difluorophenoxy)methyl)-2- azaspiro[3.5]nonan-2-yl)-2-oxoethyl)-2- (2,6-dioxopiperidin-3-yl)-6,7- dihydropyrrolo[3,4-f]isoindole- 1,3(2H,5H)-dione |
| E 454 | 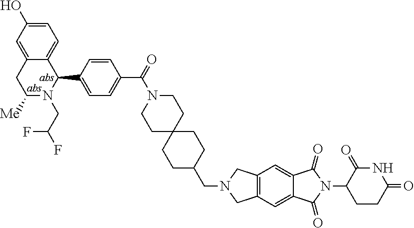 [SMILES: C[C@@H]1Cc2cc(O)ccc2[C@@H](c2ccc(C(=O)N3CCC4(CCC(CN5Cc6cc7c(cc6C5)C(=O)N(C5CCC(=O)NC5=O)C7=O)CC4)CC3)cc2)N1CC(F)F] | 6-((3-(4-((1R,3R)-2-(2,2-difluoroethyl)- 6-hydroxy-3-methyl-1,2,3,4- tetrahydroisoquinolin-1-yl)benzoyl)-3- azaspiro[5.5]undecan-9-yl)methyl)-2- (2,6-dioxopiperidin-3-yl)-6,7- dihydropyrrolo[3,4-f]isoindole- 1,3(2H,5H)-dione |
| E 455 | 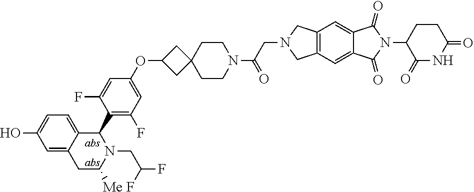 [SMILES: C[C@@H]1Cc2cc(O)ccc2[C@@H](c2c(F)cc(OC3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1S,3R)-2-(2,2- difluoroethyl)-6-hydroxy-3-methyl- 1,2,3,4-tetrahydroisoquinolin-1-yl)-3,5- difluorophenoxy)-7-azaspiro[3.5]nonan- 7-yl)-2-oxoethyl)-2-(2,6-dioxopiperidin- 3-yl)-6,7-dihydropyrrolo[3,4- f]isoindole-1,3(2H,5H)-dione |
| E 456 | 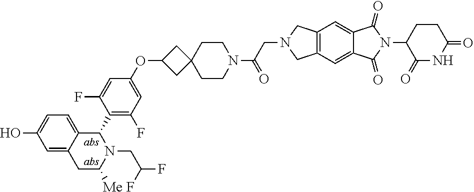 [SMILES: C[C@@H]1Cc2cc(O)ccc2[C@H](c2c(F)cc(OC3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1R,3R)-2-(2,2- difluoroethyl)-6-hydroxy-3-methyl- 1,2,3,4-tetrahydroisoquinolin-1-yl)-3,5- difluorophenoxy)-7-azaspiro[3.5]nonan- 7-yl)-2-oxoethyl)-2-(2,6-dioxopiperidin- 3-yl)-6,7-dihydropyrrolo[3,4- f]isoindole-1,3(2H,5H)-dione |
| E 457 | 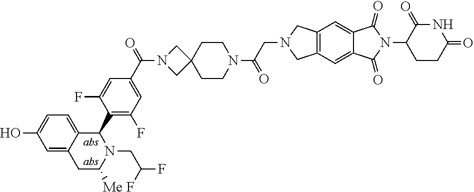 [SMILES: C[C@@H]1Cc2cc(O)ccc2[C@@H](c2c(F)cc(C(=O)N3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1S,3R)-2-(2,2- difluoroethyl)-6-hydroxy-3-methyl- 1,2,3,4-tetrahydroisoquinolin-1-yl)-3,5- difluorobenzoyl)-2,7- diazaspiro[3.5]nonan-7-yl)-2-oxoethyl)- 2-(2,6-dioxopiperidin-3-yl)-6,7- dihydropyrrolo[3,4-f]isoindole- 1,3(2H,5H)-dione |
| E 458 | 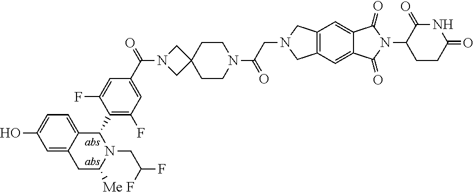 [SMILES: C[C@@H]1Cc2cc(O)ccc2[C@H](c2c(F)cc(C(=O)N3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((1R,3R)-2-(2,2- difluoroethyl)-6-hydroxy-3-methyl- 1,2,3,4-tetrahydroisoquinolin-1-yl)-3,5- difluorobenzoyl)-2,7- diazaspiro[3.5]nonan-7-yl)-2-oxoethyl)- 2-(2,6-dioxopiperidin-3-yl)-6,7- dihydropyrrolo[3,4-f]isoindole- 1,3(2H,5H)-dione |
| E 459 | 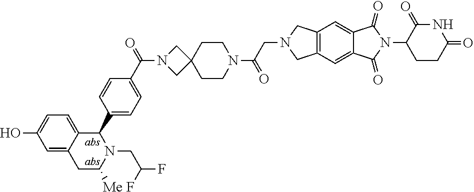 [SMILES: C[C@@H]1Cc2cc(O)ccc2[C@@H](c2ccc(C(=O)N3CC4(CCN(C(=O)CN5Cc6cc7c(cc6C5)C(=O)N(C5CCC(=O)NC5=O)C7=O)CC4)C3)cc2)N1CC(F)F] | 6-(2-(2-(4-((1R,3R)-2-(2,2- difluoroethyl)-6-hydroxy-3-methyl- 1,2,3,4-tetrahydroisoquinolin-1- yl)benzoyl)-2,7-diazaspiro[3.5]nonan-7- yl)-2-oxoethyl)-2-(2,6-dioxopiperidin-3- yl)-6,7-dihydropyrrolo[3,4-f]isoindole- 1,3(2H,5H)-dione |
| E 460 | 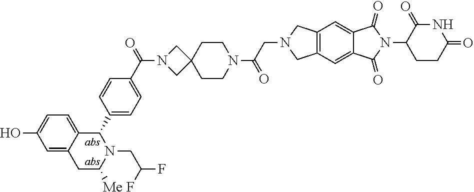 [SMILES: C[C@@H]1Cc2cc(O)ccc2[C@H](c2ccc(C(=O)N3CC4(CCN(C(=O)CN5Cc6cc7c(cc6C5)C(=O)N(C5CCC(=O)NC5=O)C7=O)CC4)C3)cc2)N1CC(F)F] | 6-(2-(2-(4-((1S,3R)-2-(2,2- difluoroethyl)-6-hydroxy-3-methyl- 1,2,3,4-tetrahydroisoquinolin-1- yl)benzoyl)-2,7-diazaspiro[3.5]nonan-7- yl)-2-oxoethyl)-2-(2,6-dioxopiperidin-3- yl)-6,7-dihydropyrrolo[3,4-f]isoindole- 1,3(2H,5H)-dione |
| E 461 | 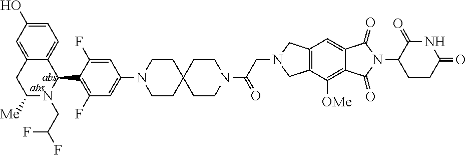 [SMILES: COc1c2c(cc3c1C(=O)N(C1CCC(=O)NC1=O)C3=O)CN(CC(=O)N1CCC3(CC1)CCN(c1cc(F)c([C@@H]4c5ccc(O)cc5C[C@@H](C)N4CC(F)F)c(F)c1)CC3)C2] | 3-(6-(2-(9-(4-((1S,3R)-2-(2,2- difluoroethyl)-6-hydroxy-3-methyl- 1,2,3,4-tetrahydroisoquinolin-1-yl)-3,5- difluorophenyl)-3,9- diazaspiro[5.5]undecan-3-yl)-2- oxoethyl)-4-methoxy-1-oxo-3,5,6,7- tetrahydropyrrolo[3,4-f]isoindol-2(1H)- yl)piperidine-2,6-dione |
| E 462 | 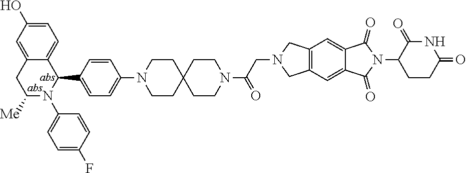 [SMILES: C[C@@H]1Cc2cc(O)ccc2[C@@H](c2ccc(N3CCC4(CCN(C(=O)CN5Cc6cc7c(cc6C5)C(=O)N(C5CCC(=O)NC5=O)C7=O)CC4)CC3)cc2)N1c1ccc(F)cc1] | 2-(2,6-dioxopiperidin-3-yl)-6-(2-(9-(4- ((1R,3R)-2-(4-fluorophenyl)-6-hydroxy- 3-methyl-1,2,3,4-tetrahydroisoquinolin- 1-yl)phenyl)-3,9- diazaspiro[5.5]undecan-3-yl)-2- oxoethyl)-6,7-dihydropyrrolo[3,4- f]isoindole-1,3(2H,5H)-dione |
| E 463 | 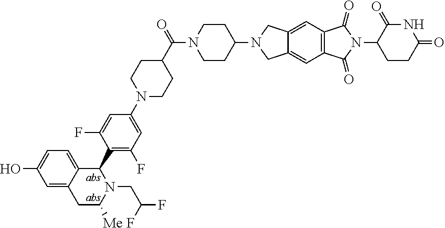 [SMILES: C[C@@H]1Cc2cc(O)ccc2[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((1S,3R)-2-(2,2- difluoroethyl)-6-hydroxy-3-methyl- 1,2,3,4-tetrahydroisoquinolin-1-yl)-3,5- difluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| E 464 | 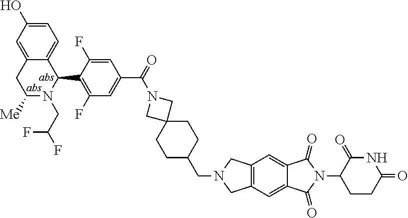 [SMILES: C[C@@H]1Cc2cc(O)ccc2[C@@H](c2c(F)cc(C(=O)N3CC4(CCC(CN5Cc6cc7c(cc6C5)C(=O)N(C5CCC(=O)NC5=O)C7=O)CC4)C3)cc2F)N1CC(F)F] | 6-((2-(4-((1S,3R)-2-(2,2-difluoroethyl)- 6-hydroxy-3-methyl-1,2,3,4- tetrahydroisoquinolin-1-yl)-3,5- difluorobenzoyl)-2-azaspiro[3.5]nonan- 7-yl)methyl)-2-(2,6-dioxopiperidin-3- yl)-6,7-dihydropyrrolo[3,4-f]isoindole- 1,3(2H,5H)-dione |
| E 465 | 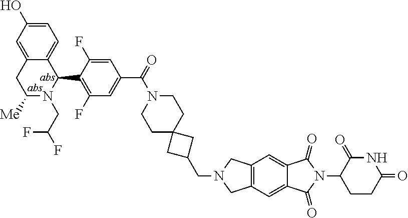 [SMILES: C[C@@H]1Cc2cc(O)ccc2[C@@H](c2c(F)cc(C(=O)N3CCC4(CC3)CC(CN3Cc5cc6c(cc5C3)C(=O)N(C3CCC(=O)NC3=O)C6=O)C4)cc2F)N1CC(F)F] | 6-((7-(4-((1S,3R)-2-(2,2-difluoroethyl)- 6-hydroxy-3-methyl-1,2,3,4- tetrahydroisoquinolin-1-yl)-3,5- difluorobenzoyl)-7-azaspiro[3.5]nonan- 2-yl)methyl)-2-(2,6-dioxopiperidin-3- yl)-6,7-dihydropyrrolo[3,4-f]isoindole- 1,3(2H,5H)-dione |
| E 466 | 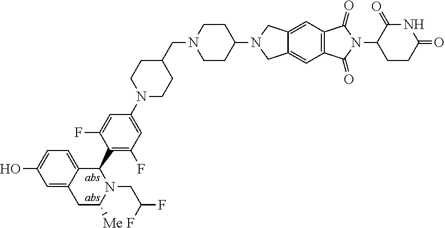 [SMILES: C[C@@H]1Cc2cc(O)ccc2[C@@H](c2c(F)cc(N3CCC(CN4CCC(N5Cc6cc7c(cc6C5)C(=O)N(C5CCC(=O)NC5=O)C7=O)CC4)CC3)cc2F)N1CC(F)F] | 6-(1-((1-(4-((1S,3R)-2-(2,2- difluoroethyl)-6-hydroxy-3-methyl- 1,2,3,4-tetrahydroisoquinolin-1-yl)-3,5- difluorophenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| E 467 | 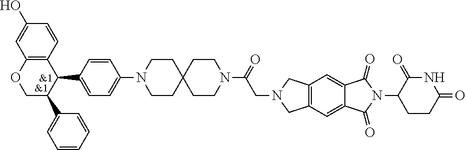 [SMILES: O=C1CCC(N2C(=O)c3cc4c(cc3C2=O)CN(CC(=O)N2CCC3(CC2)CCN(c2ccc([C@@H]5c6ccc(O)cc6OC[C@@H]5c5ccccc5)cc2)CC3)C4)C(=O)N1] | 2-(2,6-dioxopiperidin-3-yl)-6-(2-(9-(4- (7-hydroxy-3-phenylchroman-4- yl)phenyl)-3,9-diazaspiro[5.5]undecan- 3-yl)-2-oxoethyl)-6,7- dihydropyrrolo[3,4-f]isoindole-1, 3(2H,5H)-dione |
| E 468 | 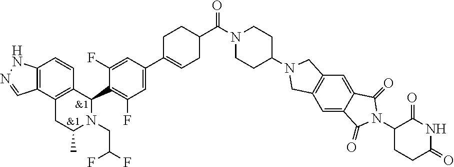 [SMILES: C[C@@H]1Cc2c(ccc3[nH]ncc23)[C@@H](c2c(F)cc(C3=CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4′-((6S,8R)-7-(2,2-difluoroethyl)- 8-methyl-6,7,8,9-tetrahydro-3H- pyrazolo[4,3-f]isoquinolin-6-yl)-3′,5′- difluoro-2,3,4,5-tetrahydro-[1,1′- biphenyl]-4-carbonyl)piperidin-4-yl)-2- (2,6-dioxopiperidin-3-yl)-6,7- dihydropyrrolo[3,4-f]isoindole- 1,3(2H,5H)-dione |
| E 469 | 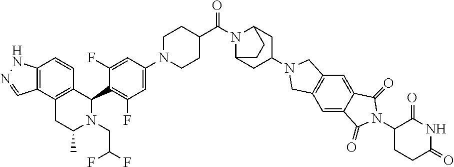 [SMILES: C[C@@H]1Cc2c(ccc3[nH]ncc23)[C@@H](c2c(F)cc(N3CCC(C(=O)N4C5CCC4CC(N4Cc6cc7c(cc6C4)C(=O)N(C4CCC(=O)NC4=O)C7=O)C5)CC3)cc2F)N1CC(F)F] | 6-(8-(1-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperidine-4-carbonyl)- 8-azabicyclo[3.2.1]octan-3-yl)-2-(2,6- dioxopiperidin-3-yl)-6,7- dihydropyrrolo[3,4-f]isoindole- 1,3(2H,5H)-dione |
| E 470 | 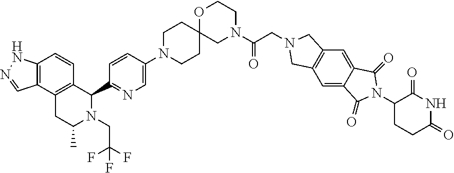 [SMILES: C[C@@H]1Cc2c(ccc3[nH]ncc23)[C@@H](c2ccc(N3CCC4(CC3)CN(C(=O)CN3Cc5cc6c(cc5C3)C(=O)N(C3CCC(=O)NC3=O)C6=O)CCO4)cn2)N1CC(F)(F)F] | 2-(2,6-dioxopiperidin-3-yl)-6-(2-(9-(6- ((6S,8R)-8-methyl-7-(2,2,2- trifluoroethyl)-6,7,8,9-tetrahydro-3H- pyrazolo[4,3-f]isoquinolin-6-yl)pyridin- 3-yl)-1-oxa-4,9-diazaspiro[5.5]undecan- 4-yl)-2-oxoethyl)-6,7- dihydropyrrolo[3,4-f]isoindole- 1,3(2H,5H)-dione |
| E 471 | 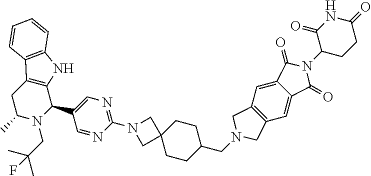 [SMILES: C[C@@H]1Cc2c([nH]c3ccccc23)[C@@H](c2cnc(N3CC4(CCC(CN5Cc6cc7c(cc6C5)C(=O)N(C5CCC(=O)NC5=O)C7=O)CC4)C3)nc2)N1CC(C)(C)F] | 2-(2,6-dioxopiperidin-3-yl)-6-((2-(5- ((1R,3R)-2-(2-fluoro-2-methylpropyl)- 3-methyl-2,3,4,9-tetrahydro-1H- pyrido[3,4-b]indol-1-yl)pyrimidin-2-yl)- 2-azaspiro[3.5]nonan-7-yl)methyl)-6,7- dihydropyrrolo[3,4-f]isoindole- 1,3(2H,5H)-dione |
| E 472 | 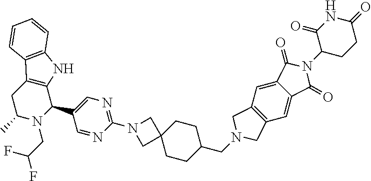 [SMILES: C[C@@H]1Cc2c([nH]c3ccccc23)[C@@H](c2cnc(N3CC4(CCC(CN5Cc6cc7c(cc6C5)C(=O)N(C5CCC(=O)NC5=O)C7=O)CC4)C3)nc2)N1CC(F)F] | 6-((2-(5-((1R,3R)-2-(2,2-difluoroethyl)- 3-methyl-2,3,4,9-tetrahydro-1H- pyrido[3,4-b]indol-1-yl)pyrimidin-2-yl)- 2-azaspiro[3.5]nonan-7-yl)methyl)-2- (2,6-dioxopiperidin-3-yl)-6,7- dihydropyrrolo[3,4-f]isoindole- 1,3(2H,5H)-dione |
| E 473 | 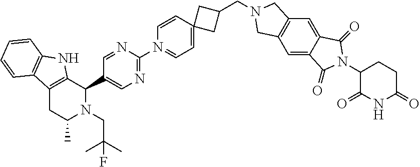 | 2-(2,6-dioxopiperidin-3-yl)-6-((7-(5- ((1R,3R)-2-(2-fluoro-2-methylpropyl)- 3-methyl-2,3,4,9-tetrahydro-1H- pyrido[3,4-b]indol-1-yl)pyrimidin-2-yl)- 7-azaspiro[3.5]nonan-2-yl)methyl)-6,7- dihydropyrrolo[3,4-f]isoindole- 1,3(2H,5H)-dione |
| E 474 | 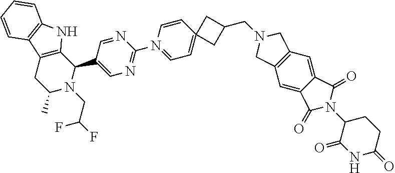 | 6-((7-(5-((1R,3R)-2-(2,2-difluoroethyl)- 3-methyl-2,3,4,9-tetrahydro-1H- pyrido[3,4-b]indol-1-yl)pyrimidin-2-yl)- 7-azaspiro[3.5]nonan-2-yl)methyl)-2- (2,6-dioxopiperidin-3-yl)-6,7- dihydropyrrolo[3,4-f]isoindole- 1,3(2H,5H)-dione |
| E 475 | 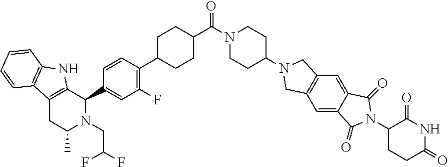 [SMILES: C[C@@H]1Cc2c([nH]c3ccccc23)[C@@H](c2ccc(C3CCC(C(=O)N4CCC(N5Cc6cc7c(cc6C5)C(=O)N(C5CCC(=O)NC5=O)C7=O)CC4)CC3)c(F)c2)N1CC(F)F] | 6-(1-(4-(4-((1R,3R)-2-(2,2- difluoroethyl)-3-methyl-2,3,4,9- tetrahydro-1H-pyrido[3,4-b]indol-1-yl)- 2-fluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 476 | 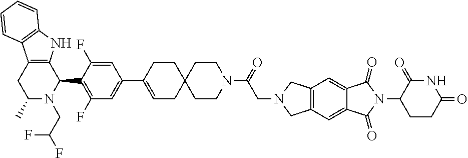 [SMILES: C[C@@H]1Cc2c([nH]c3ccccc23)[C@@H](c2c(F)cc(C3=CCC4(CC3)CCN(C(=O)CN3Cc5cc6c(cc5C3)C(=O)N(C3CCC(=O)NC3=O)C6=O)CC4)cc2F)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3,5-difluorophenyl)-3- azaspiro[5.5]undec-8-en-3-yl)-2- oxoethyl)-2-(2,6-dioxopiperidin-3-yl)- 6,7-dihydropyrrolo[3,4-f]isoindole- 1,3(2H,5H)-dione |
| E 477 | g 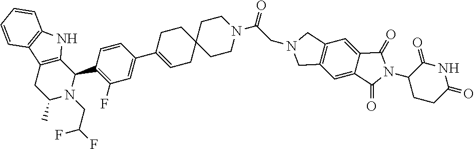 [SMILES: C[C@@H]1Cc2c([nH]c3ccccc23)[C@@H](c2ccc(C3=CCC4(CC3)CCN(C(=O)CN3Cc5cc6c(cc5C3)C(=O)N(C3CCC(=O)NC3=O)C6=O)CC4)cc2F)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3-fluorophenyl)-3-azaspiro[5.5]undec-8- en-3-yl)-2-oxoethyl)-2-(2,6- dioxopiperidin-3-yl)-6,7- dihydropyrrolo[3,4-f]isoindole- 1,3(2H,5H)-dione |
| E 478 | 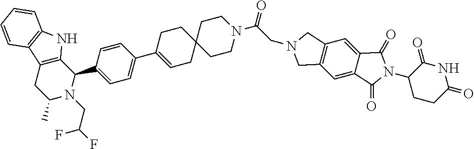 [SMILES: C[C@@H]1Cc2c([nH]c3ccccc23)[C@@H](c2ccc(C3=CCC4(CC3)CCN(C(=O)CN3Cc5cc6c(cc5C3)C(=O)N(C3CCC(=O)NC3=O)C6=O)CC4)cc2)N1CC(F)F] | 6-(2-(9-(4-((1R,3R)-2-(2,2- difluoroethyl)-3-methyl-2,3,4,9- tetrahydro-1H-pyrido[3,4-b]indol-1- yl)phenyl)-3-azaspiro[5.5]undec-8-en-3- yl)-2-oxoethyl)-2-(2,6-dioxopiperidin-3- yl)-6,7-dihydropyrrolo[3,4-f]isoindole- 1,3(2H,5H)-dione |
| E 479 | 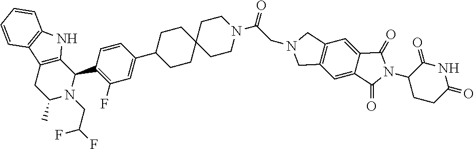 [SMILES: C[C@@H]1Cc2c([nH]c3ccccc23)[C@@H](c2ccc(C3CCC4(CC3)CCN(C(=O)CN3Cc5cc6c(cc5C3)C(=O)N(C3CCC(=O)NC3=O)C6=O)CC4)cc2F)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3-fluorophenyl)-3- azaspiro[5.5]undecan-3-yl)-2-oxoethyl)- 2-(2,6-dioxopiperidin-3-yl)-6,7- dihydropyrrolo[3,4-f]isoindole- 1,3(2H,5H)-dione |
| E 480 | 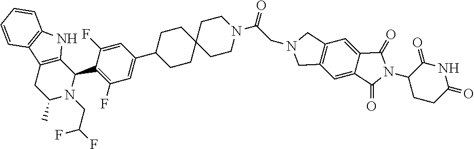 [SMILES: C[C@@H]1Cc2c([nH]c3ccccc23)[C@@H](c2c(F)cc(C3CCC4(CC3)CCN(C(=O)CN3Cc5cc6c(cc5C3)C(=O)N(C3CCC(=O)NC3=O)C6=O)CC4)cc2F)N1CC(F)F] | 6-(2-(9-(4-((1R,3R)-2-(2,2- difluoroethyl)-3-methyl-2,3,4,9- tetrahydro-1H-pyrido[3,4-b]indol-1-yl)- 3,5-difluorophenyl)-3- azaspiro[5.5]undecan-3-yl)-2-oxoethyl)- 2-(2,6-dioxopiperidin-3-yl)-6,7- dihydropyrrolo[3,4-f]isoindole- 1,3(2H,5H)-dione |
| E 481 | 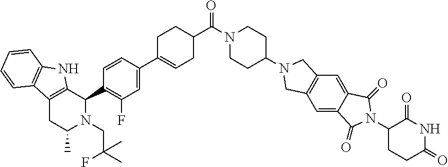 [SMILES: C[C@@H]1Cc2c([nH]c3ccccc23)[C@@H](c2ccc(C3=CCC(C(=O)N4CCC(N5Cc6cc7c(cc6C5)C(=O)N(C5CCC(=O)NC5=O)C7=O)CC4)CC3)cc2F)N1CC(C)(C)F] | 2-(2,6-dioxopiperidin-3-yl)-6-(1-(3′- fluoro-4′-((1R,3R)-2-(2-fluoro-2- methylpropyl)-3-methyl-2,3,4,9- tetrahydro-1H-pyrido[3,4-b]indol-1-yl)- 2,3,4,5-tetrahydro-[1,1′-biphenyl]-4- carbonyl)piperidin-4-yl)-6,7- dihydropyrrolo[3,4-f]isoindole- 1,3(2H,5H)-dione |
| E 482 | 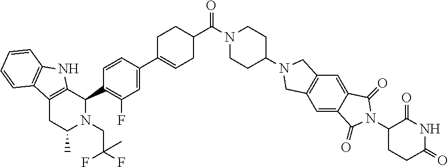 [SMILES: C[C@@H]1Cc2c([nH]c3ccccc23)[C@@H](c2ccc(C3=CCC(C(=O)N4CCC(N5Cc6cc7c(cc6C5)C(=O)N(C5CCC(=O)NC5=O)C7=O)CC4)CC3)cc2F)N1CC(C)(F)F] | 6-(1-(4′-((1R,3R)-2-(2,2- difluoropropyl)-3-methyl-2,3,4,9- tetrahydro-1H-pyrido[3,4-b]indol-1-yl)- 3′-fluoro-2,3,4,5-tetrahydro-[1,1′- biphenyl]-4-carbonyl)piperidin-4-yl)-2- (2,6-dioxopiperidin-3-yl)-6,7- dihydropyrrolo[3,4-f]isoindole- 1,3(2H,5H)-dione |
| E 483 | 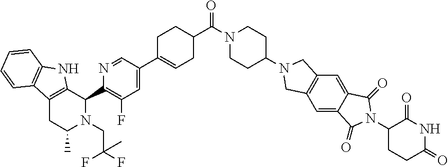 [SMILES: C[C@@H]1Cc2c([nH]c3ccccc23)[C@@H](c2ncc(C3=CCC(C(=O)N4CCC(N5Cc6cc7c(cc6C5)C(=O)N(C5CCC(=O)NC5=O)C7=O)CC4)CC3)cc2F)N1CC(C)(F)F] | 6-(1-(4-(6-((1S,3R)-2-(2,2- difluoropropyl)-3-methyl-2,3,4,9- tetrahydro-1H-pyrido[3,4-b]indol-1-yl)- 5-fluoropyridin-3-yl)cyclohex-3-ene-1- carbonyl)piperidin-4-yl)-2-(2,6- dioxopiperidin-3-yl)-6,7- dihydropyrrolo[3,4-f]isoindole- 1,3(2H,5H)-dione |
| E 484 | 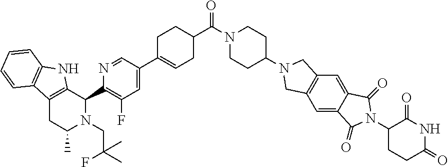 [SMILES: C[C@@H]1Cc2c([nH]c3ccccc23)[C@@H](c2ncc(C3=CCC(C(=O)N4CCC(N5Cc6cc7c(cc6C5)C(=O)N(C5CCC(=O)NC5=O)C7=O)CC4)CC3)cc2F)N1CC(C)(C)F] | 2-(2,6-dioxopiperidin-3-yl)-6-(1-(4-(5- fluoro-6-((1S,3R)-2-(2-fluoro-2- methylpropyl)-3-methyl-2,3,4,9- tetrahydro-1H-pyrido[3,4-b]indol-1- yl)pyridin-3-yl)cyclohex-3-ene-1- carbonyl)piperidin-4-yl)-6,7- dihydropyrrolo[3,4-f]isoindole- 1,3(2H,5H)-dione |
| E 485 | 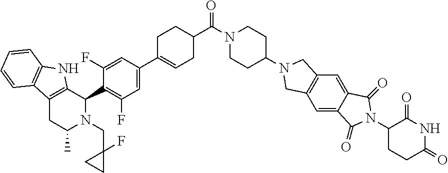 [SMILES: C[C@@H]1Cc2c([nH]c3ccccc23)[C@@H](c2c(F)cc(C3=CCC(C(=O)N4CCC(N5Cc6cc7c(cc6C5)C(=O)N(C5CCC(=O)NC5=O)C7=O)CC4)CC3)cc2F)N1CC1(F)CC1] | 6-(1-(3′,5′-difluoro-4′-((1R,3R)-2-((1- fluorocyclopropyl)methyl)-3-methyl- 2,3,4,9-tetrahydro-1H-pyrido[3,4- b]indol-1-yl)-2,3,4,5-tetrahydro-[1,1′- biphenyl]-4-carbonyl)piperidin-4-yl)-2- (2,6-dioxopiperidin-3-yl)-6,7- dihydropyrrolo[3,4-f]isoindole- 1,3(2H,5H)-dione |
| E 486 | 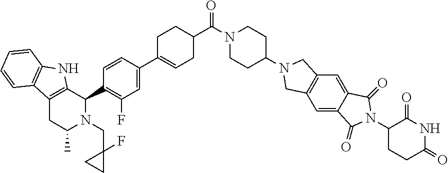 [SMILES: C[C@@H]1Cc2c([nH]c3ccccc23)[C@@H](c2ccc(C3=CCC(C(=O)N4CCC(N5Cc6cc7c(cc6C5)C(=O)N(C5CCC(=O)NC5=O)C7=O)CC4)CC3)cc2F)N1CC1(F)CC1] | 2-(2,6-dioxopiperidin-3-yl)-6-(1-(3′- fluoro-4′-((1R,3R)-2-((1- fluorocyclopropyl)methyl)-3-methyl- 2,3,4,9-tetrahydro-1H-pyrido[3,4- b]indol-1-yl)-2,3,4,5-tetrahydro-[1,1′- biphenyl]-4-carbonyl)piperidin-4-yl)- 6,7-dihydropyrrolo[3,4-f]isoindole- 1,3(2H,5H)-dione |
| E 487 | 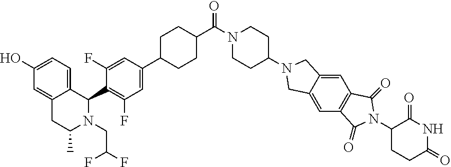 [SMILES: C[C@@H]1Cc2cc(O)ccc2[C@@H](c2c(F)cc(C3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4-(4-((1S,3R)-2-(2,2- difluoroethyl)-6-hydroxy-3-methyl- 1,2,3,4-tetrahydroisoquinolin-1-yl)-3,5- difluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| E 488 | 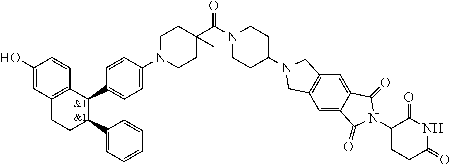 [SMILES: CC1(C(=O)N2CCC(N3Cc4cc5c(cc4C3)C(=O)N(C3CCC(=O)NC3=O)C5=O)CC2)CCN(c2ccc([C@@H]3c4ccc(O)cc4CC[C@@H]3c3ccccc3)cc2)CC1] | rac-2-(2,6-dioxopiperidin-3-yl)-6-(1-(1- (4-((1R,2S)-6-hydroxy-2-phenyl- 1,2,3,4-tetrahydronaphthalen-1- yl)phenyl)-4-methylpiperidine-4- carbonyl)piperidin-4-yl)-6,7- dihydropyrrolo[3,4-f]isoindole- 1,3(2H,5H)-dione |
| E 489 | 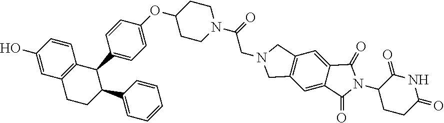 [SMILES: O=C1CCC(N2C(=O)c3cc4c(cc3C2=O)CN(CC(=O)N2CCC(Oc3ccc([C@@H]5c6ccc(O)cc6CC[C@@H]5c5ccccc5)cc3)CC2)C4)C(=O)N1] | 2-(2,6-dioxopiperidin-3-yl)-6-(2-(4-(4- ((1R,2S)-6-hydroxy-2-phenyl-1,2,3,4- tetrahydronaphthalen-1- yl)phenoxy)piperidin-1-yl)-2-oxoethyl)- 6,7-dihydropyrrolo[3,4-f]isoindole- 1,3(2H,5H)-dione |
| E 490 | 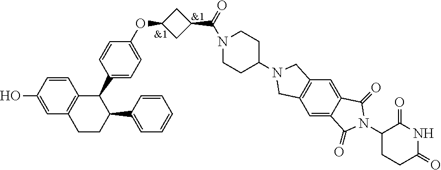 [SMILES: O=C1CCC(N2C(=O)c3cc4c(cc3C2=O)CN(C2CCN(C(=O)[C@H]3C[C@@H](Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(1- ((1SR,3SR)-3-(4-((1R,2S)-6-hydroxy-2- phenyl-1,2,3,4-tetrahydronaphthalen-1- yl)phenoxy)cyclobutane-1- carbonyl)piperidin-4-yl)-6,7- dihydropyrrolo[3,4-f]isoindole- 1,3(2H,5H)-dione |
| E 491 | 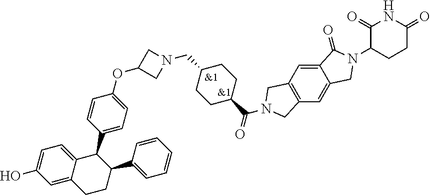 [SMILES: O=C1CCC(N2Cc3cc4c(cc3C2=O)CN(C(=O)[C@H]2CC[C@H](CN3CC(Oc5ccc([C@@H]6c7ccc(O)cc7CC[C@@H]6c6ccccc6)cc5)C3)CC2)C4)C(=O)N1] | 3-(6-((1RS,4SR)-4-((3-(4-((1R,2S)-6- hydroxy-2-phenyl-1,2,3,4- tetrahydronaphthalen-1- yl)phenoxy)azetidin-1- yl)methyl)cyclohexane-1-carbonyl)-1- oxo-3,5,6,7-tetrahydropyrrolo[3,4- flisoindol-2(1H)-yl)piperidine-2,6-dione |
| E 492 | 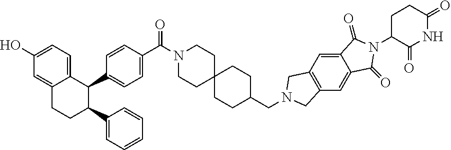 [SMILES: O=C1CCC(N2C(=O)c3cc4c(cc3C2=O)CN(CC2CCC3(CC2)CCN(C(=O)c2ccc([C@@H]5c6ccc(O)cc6CC[C@@H]5c5ccccc5)cc2)CC3)C4)C(=O)N1] | 2-(2,6-dioxopiperidin-3-yl)-6-((3-(4- ((1R,2S)-6-hydroxy-2-phenyl-1,2,3,4- tetrahydronaphthalen-1-yl)benzoyl)-3- azaspiro[5.5]undecan-9-yl)methyl)-6,7- dihydropyrrolo[3,4-f]isoindole- 1,3(2H,5H)-dione |
| E 493 | 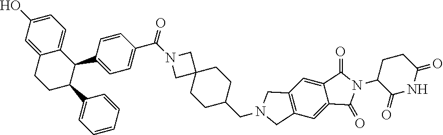 [SMILES: O=C1CCC(N2C(=O)c3cc4c(cc3C2=O)CN(CC2CCC3(CC2)CN(C(=O)c2ccc([C@@H]5c6ccc(O)cc6CC[C@@H]5c5ccccc5)cc2)C3)C4)C(=O)N1] | 2-(2,6-dioxopiperidin-3-yl)-6-((2-(4- ((1R,2S)-6-hydroxy-2-phenyl-1,2,3,4- tetrahydronaphthalen-1-yl)benzoyl)-2- azaspiro[3.5]nonan-7-yl)methyl)-6,7- dihydropyrrolo[3,4-f]isoindole- 1,3(2H,5H)-dione |
| E 494 | 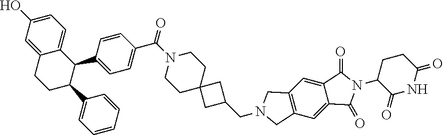 [SMILES: O=C1CCC(N2C(=O)c3cc4c(cc3C2=O)CN(CC2CC3(CCN(C(=O)c5ccc([C@@H]6c7ccc(O)cc7CC[C@@H]6c6ccccc6)cc5)CC3)C2)C4)C(=O)N1] | 2-(2,6-dioxopiperidin-3-yl)-6-((7-(4- ((1R,2S)-6-hydroxy-2-phenyl-1,2,3,4- tetrahydronaphthalen-1-yl)benzoyl)-7- azaspiro[3.5]nonan-2-yl)methyl)-6,7- dihydropyrrolo[3,4-f]isoindole- 1,3(2H,5H)-dione |
| E 495 | 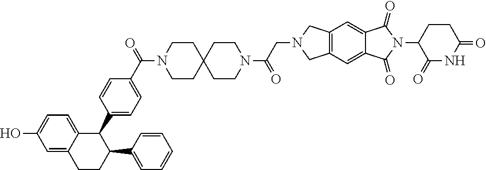 [SMILES: O=C1CCC(N2C(=O)c3cc4c(cc3C2=O)CN(CC(=O)N2CCC3(CC2)CCN(C(=O)c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4- ((1R,2S)-6-hydroxy-2-phenyl-1,2,3,4- tetrahydronaphthalen-1-yl)benzoyl)-3,9- diazaspiro[5.5]undecan-3-yl)-2- oxoethyl)-6,7-dihydropyrrolo[3,4- f]isoindole-1,3(2H,5H)-dione |
| E 496 | 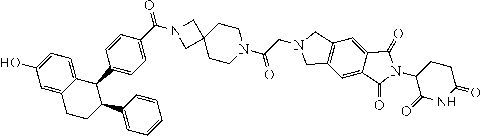 [SMILES: O=C1CCC(N2C(=O)c3cc4c(cc3C2=O)CN(CC(=O)N2CCC3(CC2)CN(C(=O)c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4- ((1R,2S)-6-hydroxy-2-phenyl-1,2,3,4- tetrahydronaphthalen-1-yl)benzoyl)-2,7- diazaspiro[3.5]nonan-7-yl)-2-oxoethyl)- 6,7-dihydropyrrolo[3,4-f]isoindole- 1,3(2H,5H)-dione |
| E 497 | 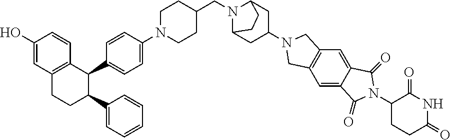 [SMILES: O=C1CCC(N2C(=O)c3cc4c(cc3C2=O)CN(C2CC3CCC(C2)N3CC2CCN(c3ccc([C@@H]5c6ccc(O)cc6CC[C@@H]5c5ccccc5)cc3)CC2)C4)C(=O)N1] | 2-(2,6-dioxopiperidin-3-yl)-6-(8-((1-(4- ((1R,2S)-6-hydroxy-2-phenyl-1,2,3,4- tetrahydronaphthalen-1- yl)phenyl)piperidin-4-yl)methyl)-8- azabicyclo[3.2.1]octan-3-yl)-6,7- dihydropyrrolo[3,4-f]isoindole- 1,3(2H,5H)-dione |
| E 498 | 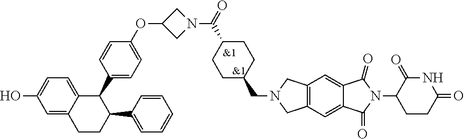 [SMILES: O=C1CCC(N2C(=O)c3cc4c(cc3C2=O)CN(C[C@H]2CC[C@H](C(=O)N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6- (((IRS,4SR)-4-(3-(4-((1R,2S)-6- hydroxy-2-phenyl-1,2,3,4- tetrahydronaphthalen-1- yl)phenoxy)azetidine-1- carbonyl)cyclohexyl)methyl)-6,7- dihydropyrrolo[3,4-f]isoindole- 1,3(2H,5H)-dione |
| E 499 | 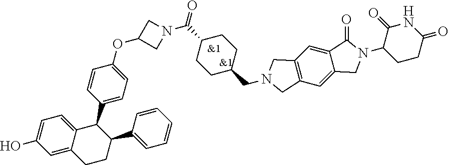 [SMILES: O=C1CCC(N2Cc3cc4c(cc3C2=O)CN(C[C@H]2CC[C@H](C(=O)N3CC(Oc5ccc([C@@H]6c7ccc(O)cc7CC[C@@H]6c6ccccc6)cc5)C3)CC2)C4)C(=O)N1] | 3-(6-(((IRS,4SR)-4-(3-(4-((1R,2S)-6- hydroxy-2-phenyl-1,2,3,4- tetrahydronaphthalen-1- yl)phenoxy)azetidine-1- carbonyl)cyclohexyl)methyl)-1-oxo- 3,5,6,7-tetrahydropyrrolo[3,4-f]isoindol- 2(1H)-yl)piperidine-2,6-dione |
| E 500 | 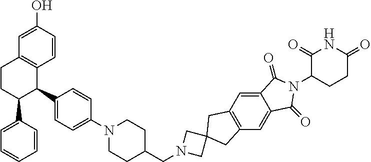 [SMILES: O=C1CCC(N2C(=O)c3cc4c(cc3C2=O)CC2(C4)CN(CC3CCN(c4ccc([C@@H]5c6ccc(O)cc6CC[C@@H]5c5ccccc5)cc4)CC3)C2)C(=O)N1] | 2′-(2,6-dioxopiperidin-3-yl)-1-((1-(4- ((1R,2S)-6-hydroxy-2-phenyl-1,2,3,4- tetrahydronaphthalen-1- yl)phenyl)piperidin-4-yl)methyl)-5′,7′- dihydro-1′H-spiro[azetidine-3,6′- cyclopenta[f]isoindole]-1′,3′(2′H)-dione |
| E 501 | 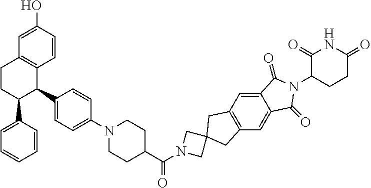 [SMILES: O=C1CCC(N2C(=O)c3cc4c(cc3C2=O)CC2(C4)CN(C(=O)C3CCN(c4ccc([C@@H]5c6ccc(O)cc6CC[C@@H]5c5ccccc5)cc4)CC3)C2)C(=O)N1] | 2′-(2,6-dioxopiperidin-3-yl)-1-(1-(4- ((1R,2S)-6-hydroxy-2-phenyl-1,2,3,4- tetrahydronaphthalen-1- yl)phenyl)piperidine-4-carbonyl)-5′,7′- dihydro-1′H-spiro[azetidine-3,6′- cyclopenta[f]isoindole]-1′,3′(2′H)-dione |
| E 502 | 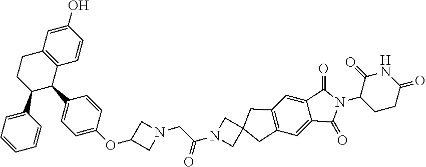 [SMILES: O=C1CCC(N2C(=O)c3cc4c(cc3C2=O)CC2(C4)CN(C(=O)CN3CC(Oc4ccc([C@@H]5c6ccc(O)cc6CC[C@@H]5c5ccccc5)cc4)C3)C2)C(=O)N1] | 2′-(2,6-dioxopiperidin-3-yl)-1-(2-(3-(4- ((1R,2S)-6-hydroxy-2-phenyl-1,2,3,4- tetrahydronaphthalen-1- yl)phenoxy)azetidin-1-yl)acetyl)-5′,7′- dihydro-1′H-spiro[azetidine-3,6′- cyclopenta[f]isoindole]-1′,3′(2′H)-dione |
| E 503 | 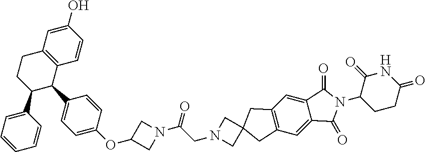 [SMILES: O=C1CCC(N2C(=O)c3cc4c(cc3C2=O)CC2(C4)CN(CC(=O)N3CC(Oc4ccc([C@@H]5c6ccc(O)cc6CC[C@@H]5c5ccccc5)cc4)C3)C2)C(=O)N1] | 2′-(2,6-dioxopiperidin-3-yl)-1-(2-(3-(4- ((1R,2S)-6-hydroxy-2-phenyl-1,2,3,4- tetrahydronaphthalen-1- yl)phenoxy)azetidin-1-yl)-2-oxoethyl)- 5′,7′-dihydro-1′H-spiro[azetidine-3,6′- cyclopenta[f]isoindole]-1′,3′(2′H)-dione |
| E 504 | 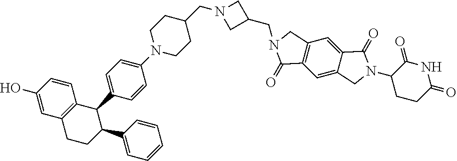 [SMILES: O=C1CCC(N2Cc3cc4c(cc3C2=O)CN(CC2CN(CC3CCN(c5ccc([C@@H]6c7ccc(O)cc7CC[C@@H]6c6ccccc6)cc5)CC3)C2)C4=O)C(=O)N1] | 2-(2,6-dioxopiperidin-3-yl)-6-((1-((1-(4- ((1R,2S)-6-hydroxy-2-phenyl-1,2,3,4- tetrahydronaphthalen-1- yl)phenyl)piperidin-4- yl)methyl)azetidin-3-yl)methyl)-2,3,6,7- tetrahydropyrrolo[3,4-f]isoindole-1,5- dione |
| E 505 | 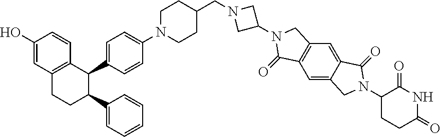 [SMILES: O=C1CCC(N2Cc3cc4c(cc3C2=O)CN(C2CN(CC3CCN(c5ccc([C@@H]6c7ccc(O)cc7CC[C@@H]6c6ccccc6)cc5)CC3)C2)C4=O)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)methyl)azetidin-3-yl)-2,3,6,7- tetrahydropyrrolo[3,4-f]isoindole-1,5- dione |
| E 506 | 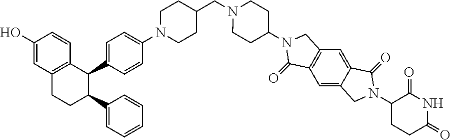 [SMILES: O=C1CCC(N2Cc3cc4c(cc3C2=O)CN(C2CCN(CC3CCN(c5ccc([C@@H]6c7ccc(O)cc7CC[C@@H]6c6ccccc6)cc5)CC3)CC2)C4=O)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)methyl)piperidin-4-yl)-2,3,6,7- tetrahydropyrrolo[3,4-f]isoindole-1,5- dione |
| E 507 | 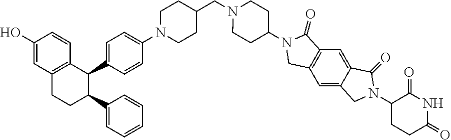 [SMILES: O=C1CCC(N2Cc3cc4c(cc3C2=O)C(=O)N(C2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-((1-(4- ((1R,2S)-6-hydroxy-2-phenyl-1,2,3,4- tetrahydronaphthalen-1- yl)phenyl)piperidin-4- yl)methyl)piperidin-4-yl)-2,3,5,6- tetrahydropyrrolo[3,4-f]isoindole-1,7- dione |
| E 508 | 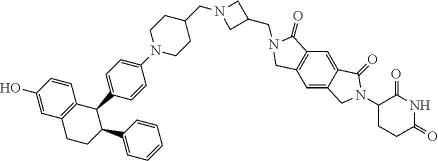 [SMILES: O=C1CCC(N2Cc3cc4c(cc3C2=O)C(=O)N(CC2CN(CC3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((1-((1-(4- ((1R,2S)-6-hydroxy-2-phenyl-1,2,3,4- tetrahydronaphthalen-1- yl)phenyl)piperidin-4- yl)methyl)azetidin-3-yl)methyl)-2,3,5,6- tetrahydropyrrolo[3,4-f]isoindole-1,7- dione |
| E 509 | 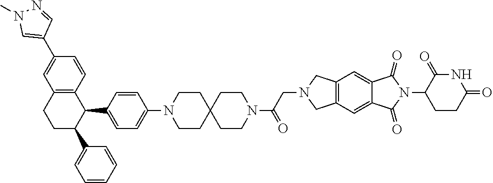 [SMILES: Cn1cc(-c2ccc3c(c2)CC[C@H](c2ccccc2)[C@@H]3c2ccc(N3CCC4(CCN(C(=O)CN5Cc6cc7c(cc6C5)C(=O)N(C5CCC(=O)NC5=O)C7=O)CC4)CC3)cc2)cn1] | 2-(2,6-dioxopiperidin-3-yl)-6-(2-(9-(4- ((1R,2S)-6-(1-methyl-1H-pyrazol-4-yl)- 2-phenyl-1,2,3,4-tetrahydronaphthalen- 1-yl)phenyl)-3,9- diazaspiro[5.5]undecan-3-yl)-2- oxoethyl)-6,7-dihydropyrrolo[3,4- f]isoindole-1,3(2H,5H)-dione |
| E 510 | 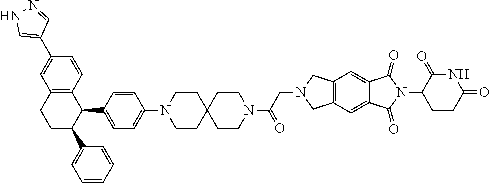 [SMILES: O=C1CCC(N2C(=O)c3cc4c(cc3C2=O)CN(CC(=O)N2CCC3(CC2)CCN(c2ccc([C@@H]5c6ccc(-c7cn[nH]c7)cc6CC[C@@H]5c5ccccc5)cc2)CC3)C4)C(=O)N1] | 2-(2,6-dioxopiperidin-3-yl)-6-(2-oxo-2- (9-(4-((1R,2S)-2-phenyl-6-(1H-pyrazol- 4-yl)-1,2,3,4-tetrahydronaphthalen-1- yl)phenyl)-3,9-diazaspiro[5.5]undecan- 3-yl)ethyl)-6,7-dihydropyrrolo[3,4- f]isoindole-1,3(2H,5H)-dione |
| E 511 | 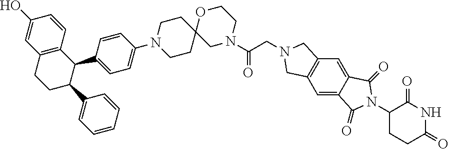 [SMILES: O=C1CCC(N2C(=O)c3cc4c(cc3C2=O)CN(CC(=O)N2CCOC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(9-(4- ((1R,2S)-6-hydroxy-2-phenyl-1,2,3,4- tetrahydronaphthalen-1-yl)phenyl)-1- oxa-4,9-diazaspiro[5.5]undecan-4-yl)-2- oxoethyl)-6,7-dihydropyrrolo[3,4- f]isoindole-1,3(2H,5H)-dione |
| E 512 | 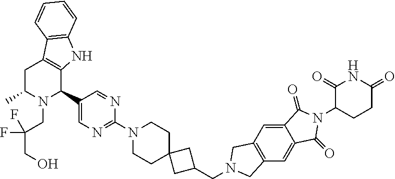 [SMILES: C[C@@H]1Cc2c([nH]c3ccccc23)[C@@H](c2cnc(N3CCC4(CC3)CC(CN3Cc5cc6c(cc5C3)C(=O)N(C3CCC(=O)NC3=O)C6=O)C4)nc2)N1CC(F)(F)CO] | 6-((7-(5-((1R,3R)-2-(2,2-difluoro-3- hydroxypropyl)-3-methyl-2,3,4,9- tetrahydro-1H-pyrido[3,4-b]indol-1- yl)pyrimidin-2-yl)-7- azaspiro[3.5]nonan-2-yl)methyl)-2-((S)- 2,6-dioxopiperidin-3-yl)-6,7- dihydropyrrolo[3,4-f]isoindole- 1,3(2H,5H)-dione |
| E 513 | 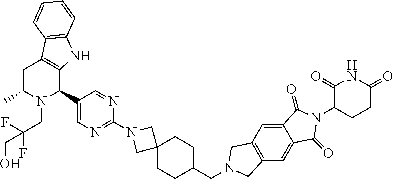 [SMILES: C[C@@H]1Cc2c([nH]c3ccccc23)[C@@H](c2cnc(N3CC4(CCC(CN5Cc6cc7c(cc6C5)C(=O)N(C5CCC(=O)NC5=O)C7=O)CC4)C3)nc2)N1CC(F)(F)CO] | 6-((2-(5-((1R,3R)-2-(2,2-difluoro-3- hydroxypropyl)-3-methyl-2,3,4,9- tetrahydro-1H-pyrido[3,4-b]indol-1- yl)pyrimidin-2-yl)-2- azaspiro[3.5]nonan-7-yl)methyl)-2-(2,6- dioxopiperidin-3-yl)-6,7- dihydropyrrolo[3,4-f]isoindole- 1,3(2H,5H)-dione |
| E 514 | 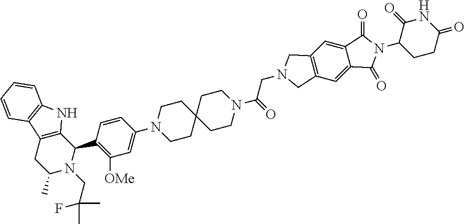 [SMILES: COc1cc(N2CCC3(CCN(C(=O)CN4Cc5cc6c(cc5C4)C(=O)N(C4CCC(=O)NC4=O)C6=O)CC3)CC2)ccc1[C@@H]1c2[nH]c3ccccc3c2C[C@@H](C)N1CC(C)(C)F] | 2-(2,6-dioxopiperidin-3-yl)-6-(2-(9-(4- ((1R,3R)-2-(2-fluoro-2-methylpropyl)- 3-methyl-2,3,4,9-tetrahydro-1H- pyrido[3,4-b]indol-1-yl)-3- methoxyphenyl)-3,9- diazaspiro[5.5]undecan-3-yl)-2- oxoethyl)-6,7-dihydropyrrolo[3,4- f]isoindole-1,3(2H,5H)-dione |
| E 515 | 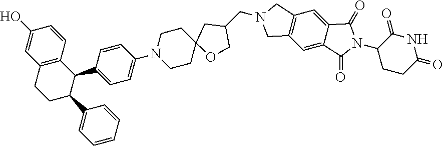 [SMILES: O=C1CCC(N2C(=O)c3cc4c(cc3C2=O)CN(CC2COC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((8-(4- ((1R,2S)-6-hydroxy-2-phenyl-1,2,3,4- tetrahydronaphthalen-1-yl)phenyl)-1- oxa-8-azaspiro[4.5]decan-3-yl)methyl)- 6,7-dihydropyrrolo[3,4-f]isoindole- 1,3(2H,5H)-dione |
| E 516 | 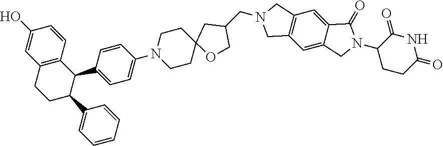 [SMILES: O=C1CCC(N2Cc3cc4c(cc3C2=O)CN(CC2COC3(CCN(c5ccc([C@@H]6c7ccc(O)cc7CC[C@@H]6c6ccccc6)cc5)CC3)C2)C4)C(=O)N1] | 3-(6-((8-(4-((1R,2S)-6-hydroxy-2- phenyl-1,2,3,4-tetrahydronaphthalen-1- yl)phenyl)-1-oxa-8-azaspiro[4.5]decan- 3-yl)methyl)-1-oxo-3,5,6,7- tetrahydropyrrolo[3,4-f]isoindol-2(1H)- yl)piperidine-2,6-dione |
| E 517 | 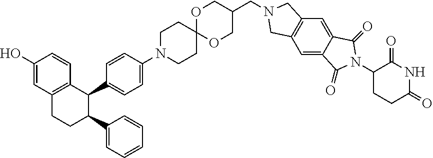 [SMILES: O=C1CCC(N2C(=O)c3cc4c(cc3C2=O)CN(CC2COC3(CCN(c5ccc([C@@H]6c7ccc(O)cc7CC[C@@H]6c6ccccc6)cc5)CC3)OC2)C4)C(=O)N1] | 2-(2,6-dioxopiperidin-3-yl)-6-((9-(4- ((1R,2S)-6-hydroxy-2-phenyl-1,2,3,4- tetrahydronaphthalen-1-yl)phenyl)-1,5- dioxa-9-azaspiro[5.5]undecan-3- yl)methyl)-6,7-dihydropyrrolo[3,4- f]isoindole-1,3(2H,5H)-dione |
| E 518 | 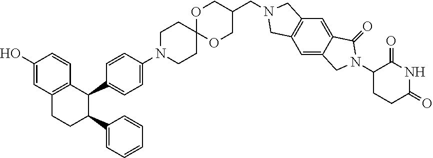 [SMILES: O=C1CCC(N2Cc3cc4c(cc3C2=O)CN(CC2COC3(CCN(c5ccc([C@@H]6c7ccc(O)cc7CC[C@@H]6c6ccccc6)cc5)CC3)OC2)C4)C(=O)N1] | 3-(6-((9-(4-((1R,2S)-6-hydroxy-2- phenyl-1,2,3,4-tetrahydronaphthalen-1- yl)phenyl)-1,5-dioxa-9- azaspiro[5.5]undecan-3-yl)methyl)-1- oxo-3,5,6,7-tetrahydropyrrolo[3,4- flisoindol-2(1H)-yl)piperidine-2,6-dione |
| E 519 | 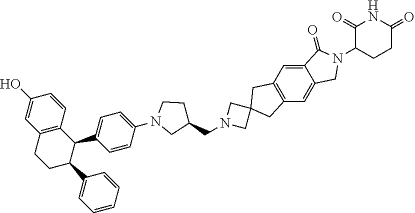 [SMILES: O=C1CCC(N2Cc3cc4c(cc3C2=O)CC2(C4)CN(C[C@@H]3CCN(c4ccc([C@@H]5c6ccc(O)cc6CC[C@@H]5c5ccccc5)cc4)C3)C2)C(=O)N1] | 3-(1-(((S)-1-(4-((1R,2S)-6-hydroxy-2- phenyl-1,2,3,4-tetrahydronaphthalen-1- yl)phenyl)pyrrolidin-3-yl)methyl)-1′- oxo-5′,7′-dihydro-1′H-spiro[azetidine- 3,6′-cyclopenta[f]isoindol]-2′(3′H)- yl)piperidine-2,6-dione |

In some embodiments, the disclosure provides a pharmaceutical composition comprising a Compound of the Disclosure and a pharmaceutically acceptable carrier or excipient.

Compounds of the Disclosure may contain an asymmetric carbon atom. In some embodiments, Compounds of the Disclosure are racemic compounds. In other embodiments, Compounds of the Disclosure are enantiomerically enriched, e.g., the enantiomeric excess or “ee” of the compound is about 5% or more as measured by chiral HPLC. In some embodiments, the ee is about 10%. In some embodiments, the ee is about 20%. In some embodiments, the ee is about 30%. In some embodiments, the ee is about 40%. In some embodiments, the ee is about 50%. In some embodiments, the ee is about 60%. In some embodiments, the ee is about 70%. In some embodiments, the ee is about 80%. In some embodiments, the ee is about 85%. In some embodiments, the ee is about 90%. In some embodiments, the ee is about 91%. In some embodiments, the ee is about 92%. In some embodiments, the ee is about 93%. In some embodiments, the ee is about 94%. In some embodiments, the ee is about 95%. In some embodiments, the ee is about 96%. In some embodiments, the ee is about 97%. In some embodiments, the ee is about 98%. In some embodiments, the ee is about 99%.

In some embodiments, the cereblon binding portion of a Compound of the Disclosure is enantiomerically enriched. In some embodiments, the cereblon binding portion of the molecule is racemic. The present disclosure encompasses all possible stereoisomeric, e.g., diastereomeric, forms of Compounds of the Disclosure. For example, all possible stereoisomers of Compounds of the Disclosure are encompassed when, e.g., the A portion of Formula I is entantiomerically enriched and the cereblon binding portion of the molecule is racemic. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure. As used herein, the pharmaceutical “pharmaceutically acceptable salt” refers to salts or zwitterionic forms of Compounds of the Disclosure. Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### Methods of Use

In some aspects, the present disclosure provides methods of degrading an ER protein in a subject, comprising administering to the subject a Compound of the Disclosure.

In some aspects, the present disclosure provides uses of a Compound of the Disclosure in the manufacture of a medicament for degrading an ER protein in a subject.

In some aspects, the present disclosure provides Compounds of the Disclosure for use in degrading an ER protein in a subject.

In some aspects, the present disclosure provides methods of treating or preventing a disease (e.g., a disease associated with degradation of an ER protein) in a subject in need thereof, comprising administering to the subject a Compound of the Disclosure (e.g., in a therapeutically effective amount).

In some aspects, the present disclosure provides methods of treating a disease (e.g., a disease associated with degradation of an ER protein) in a subject in need thereof, comprising administering to the subject a Compound of the Disclosure (e.g., in a therapeutically effective amount).

In some aspects, the present disclosure provides uses of a Compound of the Disclosure in the manufacture of a medicament for treating or preventing a disease (e.g., a disease associated with degradation of an ER protein) in a subject in need thereof.

In some aspects, the present disclosure provides uses of a Compound of the Disclosure in the manufacture of a medicament for treating a disease (e.g., a disease associated with degradation of an ER protein) in a subject in need thereof.

In some aspects, the present disclosure provides Compounds of the Disclosure for use in treating or preventing a disease (e.g., a disease associated with degradation of an ER protein) in a subject in need thereof.

In some aspects, the present disclosure provides Compounds of the Disclosure for use in treating a disease (e.g., a disease associated with degradation of an ER protein) in a subject in need thereof.

In some embodiments, the subject is a mammal.

In some embodiments, the subject is a human.

In some embodiments, the subject is a biological sample (e.g., a cell population).

In some embodiments, the disease is a cancer.

It is understood that Compounds of the Disclosure may function as ER protein degraders. Compounds of the Disclosure thus may be useful in methods of treating or preventing a disease or condition, e.g., wherein degradation of ER proteins provides a benefit, for example, cancers and proliferative diseases. The therapeutic methods of this disclosure comprise administering a therapeutically effective amount of a Compound of the Disclosure to a subject in need thereof. The present methods also encompass administering a second therapeutic agent to the subject in addition to the Compound of the Disclosure. The second therapeutic agent is selected from drugs known as useful in treating the disease or condition afflicting the subject in need thereof, e.g., a chemotherapeutic agent and/or radiation known as useful in treating a particular cancer.

The present disclosure provides Compounds of the Disclosure as ER protein degraders for the treatment of a variety of diseases and conditions wherein degradation of ER proteins has a beneficial effect. Compounds of the Disclosure typically have DC₅₀ (the drug concentration that results in 50% ER protein degradation) values of less than 100 μM, e.g., less than 50 μM, less than 25 μM, and less than 5 μM, less than about 1 μM, less than about 0.5 μM, or less than about 0.1 μM. In some embodiments, Compounds of the Disclosure have DC₅₀ values of less than about 0.05 μM. In some embodiments, Compounds of the Disclosure have DC₅₀ values of less than about 0.01 μM. In some embodiments, the present disclosure provides a method of treating a subject suffering from a disease or condition wherein the degradation of ER proteins provides a benefit comprising administering a therapeutically effective amount of a Compound of the Disclosure to a subject in need thereof.

Since Compounds of the Disclosure are degraders of ER proteins, a number of diseases and conditions mediated by ER proteins can be treated by employing these compounds. The present disclosure is thus directed generally to a method for treating a condition or disorder responsive to degradation of ER, or an isoform or mutant thereof, in an animal, e.g., a human, suffering from, or at risk of suffering from, the condition or disorder, the method comprising administering to the animal an effective amount of a Compound of the Disclosure.

The present disclosure is further directed to a method of degrading ER proteins in an animal in need thereof, said method comprising administering to the animal an effective amount a Compound of the Disclosure.

The methods of the present disclosure can be accomplished by administering a Compound of the Disclosure as the neat compound or as a pharmaceutical composition. Administration of a pharmaceutical composition, or neat compound of a Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Compound of the Disclosure and, optionally, a second therapeutic agent useful in the treatment of diseases and conditions wherein degradation of ER proteins provides a benefit, packaged separately or together, and an insert having instructions for using these active agents.

In some embodiments, a Compound of the Disclosure is administered in conjunction with a second therapeutic agent useful in the treatment of a disease or condition wherein the degradation of ER proteins provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to a subject in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more dose of the second therapeutic agent can be administered. The Compound of the Disclosure therefore can be used in conjunction with one or more second therapeutic agents, for example, but not limited to, anticancer agents.

Diseases and conditions treatable by the methods of the present disclosure include, but are not limited to, cancer and other proliferative disorders, inflammatory diseases, sepsis, autoimmune disease, and viral infection. In some embodiments, a human patient is treated with a Compound of the Disclosure, or a pharmaceutical composition comprising a Compound of the Disclosure, wherein the compound is administered in an amount sufficient to degrade ER proteins in the patient.

In some embodiments, the disease to be treated or prevented by the Compound of the Disclosure is cancer. In some embodiments, the present disclosure provides a method of treating or preventing cancer in a subject in need thereof comprising administering a therapeutically effective amount of a Compound of the Disclosure to the subject. While not being limited to a specific mechanism, in some embodiments, Compounds of the Disclosure treat or prevent cancer by degrading ER proteins. Examples of treatable cancers include, but are not limited to, any one or more of the cancers of Table I.

TABLE I

| | |
|---|---|
| adrenal cancer | lymphoepithelioma |
| acinic cell carcinoma | lymphoma |
| acoustic neuroma | acute lymphocytic leukemia |
| acral lentigious melanoma | acute myelogeous leukemia |
| acrospiroma | chronic lymphocytic leukemia |
| acute eosinophilic leukemia | liver cancer |
| acute erythroid leukemia | small cell lung cancer |
| acute lymphoblastic leukemia | non-small cell lung cancer |
| acute megakaryoblastic leukemia | MALT lymphoma |
| acute monocytic leukemia | malignant fibrous histiocytoma |
| acute promyelocytic leukemia | malignant peripheral nerve sheath tumor |
| adenocarcinoma | malignant triton tumor |
| adenoid cystic carcinoma | mantle cell lymphoma |
| adenoma | marginal zone B-cell lymphoma |
| adenomatoid odontogenic tumor | mast cell leukemia |
| adenosquamous carcinoma | mediastinal germ cell tumor |
| adipose tissue neoplasm | medullary carcinoma of the breast |
| adrenocortical carcinoma | medullary thyroid cancer, |
| adult T-cell leukemia/lymphoma | medulloblastoma |
| aggressive NK-cell leukemia | melanoma, |
| AIDS-related lymphoma | meningioma, |
| alveolar rhabdomyosarcoma | merkel cell cancer |
| alveolar soft part sarcoma | mesothelioma |
| ameloblastic fibroma | metastatic urothelial carcinoma |
| anaplastic large cell lymphoma | mixed Mullerian tumor |
| anaplastic thyroid cancer | mucinous tumor |
| angioimmunoblastic T-cell lymphoma, | multiple myeloma |
| angiomyolipoma | muscle tissue neoplasm |
| angiosarcoma | mycosis fungoides |
| astrocytoma | myxoid liposarcoma |
| atypical teratoid rhabdoid tumor | myxoma |
| B-cell chronic lymphocytic leukemia | myxosarcoma |
| B-cell prolymphocytic leukemia | nasopharyngeal carcinoma |
| B-cell lymphoma | neurinoma |
| basal cell carcinoma | neuroblastoma |
| biliary tract cancer | neurofibroma |
| bladder cancer | neuroma |
| blastoma | nodular melanoma |
| bone cancer | ocular cancer |
| Brenner tumor | oligoastrocytoma |
| Brown tumor | oligodendroglioma |
| Burkitt's lymphoma | oncocytoma |
| breast cancer | optic nerve sheath meningioma |
| brain cancer | optic nerve tumor |
| carcinoma | oral cancer |
| carcinoma in situ | osteosarcoma |
| carcinosarcoma | ovarian cancer |
| cartilage tumor | Pancoast tumor |
| cementoma | papillary thyroid cancer |
| myeloid sarcoma | paraganglioma |
| chondroma | pinealoblastoma |
| chordoma | pineocytoma |
| choriocarcinoma | pituicytoma |
| choroid plexus papilloma | pituitary adenoma |
| clear-cell sarcoma of the kidney | pituitary tumor |
| craniopharyngioma | plasmacytoma |
| cutaneous T-cell lymphoma | polyembryoma |
| cervical cancer | precursor T-lymphoblastic lymphoma |
| colorectal cancer | primary central nervous system lymphoma |
| Degos disease | primary effusion lymphoma |
| desmoplastic small round cell tumor | preimary peritoneal cancer |
| diffuse large B-cell lymphoma | prostate cancer |
| dysembryoplastic neuroepithelial tumor, | pancreatic cancer |
| dysgerminoma | pharyngeal cancer |
| embryonal carcinoma | pseudomyxoma periotonei |
| endocrine gland neoplasm | renal cell carcinoma |
| endodermal sinus tumor | renal medullary carcinoma |
| enteropathy-associated T-cell lymphoma | retinoblastoma |
| esophageal cancer | rhabdomyoma |
| fetus in fetu | rhabdomyosarcoma |
| fibroma | Richter's transformation |
| fibrosarcoma | rectal cancer |
| follicular lymphoma | sarcoma |
| follicular thyroid cancer | Schwannomatosis |
| ganglioneuroma | seminoma |
| gastrointestinal cancer | Sertoli cell tumor |
| germ cell tumor | sex cord-gonadal stromal tumor |
| gestational choriocarcinoma | signet ring cell carcinoma |
| giant cell fibroblastoma | skin cancer |
| giant cell tumor of the bone | small blue round cell tumors |
| glial tumor | small cell carcinoma |
| glioblastoma multiforme | soft tissue sarcoma |
| glioma | somatostatinoma |
| gliomatosis cerebri | soot wart |
| glucagonoma | spinal tumor |
| gonadoblastoma | splenic marginal zone lymphoma |
| granulosa cell tumor | squamous cell carcinoma |
| gynandroblastoma | synovial sarcoma |
| gallbladder cancer | Sezary's disease |
| gastric cancer | small intestine cancer |
| hairy cell leukemia | squamous carcinoma |
| hemangioblastoma | stomach cancer |
| head and neck cancer | T-cell lymphoma |
| hemangiopericytoma | testicular cancer |
| hematological malignancy | thecoma |
| hepatoblastoma | thyroid cancer |
| hepatosplenic T-cell lymphoma | transitional cell carcinoma |
| Hodgkin's lymphoma | throat cancer |
| non-Hodgkin's lymphoma | urachal cancer |
| invasive lobular carcinoma | urogenital cancer |
| intestinal cancer | urothelial carcinoma |
| kidney cancer | uveal melanoma |
| laryngeal cancer | uterine cancer |
| lentigo maligna | verrucous carcinoma |
| lethal midline carcinoma | visual pathway glioma |
| leukemia | vulvar cancer |
| leydig cell tumor | vaginal cancer |
| liposarcoma | Waldenstrom's macroglobulinemia |
| lung cancer | Warthin's tumor |
| lymphangioma | Wilms' tumor |
| lymphangiosarcoma | |

In some embodiments, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In some embodiments, the cancer is prostate cancer. In some embodiments, the cancer is breast cancer. In some embodiments, the cancer is triple-negative breast cancer (TNBC). In some embodiments, the cancer is castration-resistant prostate cancer (CRPC). In some embodiments, the cancer is KRAS-mutated or ALK-positive non-small cell lung cancer (NSCLC).

In some embodiments, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

Compounds of the Disclosure can also treat infectious and noninfectious inflammatory events and autoimmune and other inflammatory diseases by administration of an effective amount of a present compound to a mammal, in particular a human in need of such treatment. Examples of autoimmune and inflammatory diseases, disorders, and syndromes treated using the compounds and methods described herein include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendictitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

In some embodiments, the present disclosure provides a method of treating systemic inflammatory response syndromes, such as LPS-induced endotoxic shock and/or bacteria-induced sepsis by administration of an effective amount of a Compound of the Disclosure to a mammal, in particular a human in need of such treatment.

In some embodiments, the present disclosure provides a method for treating viral infections and diseases. Examples of viral infections and diseases treated using the compounds and methods described herein include episome-based DNA viruses including, but not limited to, human papillomavirus, Herpesvirus, Epstein-Barr virus, human immunodeficiency virus, hepatis B virus, and hepatitis C virus.

In some embodiments, the present disclosure provides therapeutic method of modulating protein methylation, gene expression, cell proliferation, cell differentiation and/or apoptosis in vivo in diseases mentioned above, in particular cancer, inflammatory disease, and/or viral disease is provided by administering a therapeutically effective amount of a Compound of the Disclosure to a subject in need of such therapy.

In some embodiments, the present disclosure provides a method of regulating endogenous or heterologous promoter activity by contacting a cell with a Compound of the Disclosure.

In methods of the present disclosure, a therapeutically effective amount of a Compound of the Disclosure, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the ER protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a Compound of the Disclosure can be administered in combination with a second therapeutically active agent. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In some embodiments, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DM1, cetuximab, bevacizumab, rituximab, PR064553, and 2C4. The term “antibody” includes intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SU11248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino, 17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, Cl-1033, EKB-569, GW-2016, antibodies El.l, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-lH-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Other examples of second therapeutic agents, one or more of which a Compound of the Disclosure also can be combined, include, but are not limited to: a treatment for Alzheimer's Disease, such as donepezil and rivastigmine; a treatment for Parkinson's Disease, such as L-DOPA/carbidopa, entacapone, ropinrole, pramipexole, bromocriptine, pergolide, trihexephendyl, and amantadine; an agent for treating multiple sclerosis (MS) such as beta interferon (e.g., AVONEX® and REBIF®), glatiramer acetate, and mitoxantrone; a treatment for asthma, such as albuterol and montelukast; an agent for treating schizophrenia, such as zyprexa, risperdal, seroquel, and haloperidol; an anti-inflammatory agent, such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor, such as an acetylcholinesterase inhibitor, an MAO inhibitor, an interferon, an anti-convulsant, an ion channel blocker, riluzole, or an anti-Parkinson's agent; an agent for treating cardiovascular disease, such as a beta-blocker, an ACE inhibitor, a diuretic, a nitrate, a calcium channel blocker, or a statin; an agent for treating liver disease, such as a corticosteroid, cholestyramine, an interferon, and an anti-viral agent; an agent for treating blood disorders, such as a corticosteroid, an anti-leukemic agent, or a growth factor; or an agent for treating immunodeficiency disorders, such as gamma globulin.

The above-mentioned second therapeutically active agents, one or more of which can be used in combination with a Compound of the Disclosure, are prepared and administered as described in the art.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. In some embodiments, a pharmaceutical composition comprising a Compound of the Disclosure, or a pharmaceutically acceptable salt or hydrate thereof, and a pharmaceutically acceptable carrier, is provided. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject.

Embodiment I. A method of treating a subject, the method comprising administering to the subject a therapeutically effective amount of a Compound of the Disclosure, wherein the subject has cancer, a chronic autoimmune disorder, an inflammatory condition, a proliferative disorder, sepsis, or a viral infection.

Embodiment II. The method of Embodiment I, wherein the subject has cancer.

Embodiment III. The method of Embodiment II, wherein the cancer is a cancer listed in Table I.

Embodiment IV. The method of Embodiment II, wherein the cancer is breast cancer.

Embodiment V. The method of Embodiment II, wherein the cancer is a cancer wherein the inhibition or degradation of ER provides a benefit.

Embodiment VI. The method of any one of Embodiments I-V further comprising administering a therapeutically effective amount of a second therapeutic agent useful in the treatment of the disease or condition, e.g., an immune checkpoint inhibitor or other anticancer agent.

Embodiment VII. A pharmaceutical composition comprising a Compound of the Disclosure and a pharmaceutically acceptable excipient for use in treating cancer, a chronic autoimmune disorder, an inflammatory condition, a proliferative disorder, sepsis, or a viral infection.

Embodiment VIII. The pharmaceutical composition of Embodiment VII for use in treating cancer.

Embodiment IX. The pharmaceutical composition of Embodiment VIII, wherein the cancer is a cancer listed in Table I.

Embodiment X. The pharmaceutical composition of Embodiment VIII, wherein the cancer is breast cancer.

Embodiment XI. The pharmaceutical composition of Embodiment VIII, wherein the cancer is a cancer wherein the inhibition or degradation of ER provides a benefit.

Embodiment XII. A Compound of the Disclosure for use in treatment of cancer, a chronic autoimmune disorder, an inflammatory condition, a proliferative disorder, sepsis, or a viral infection.

Embodiment XIII. The compound of Embodiment XIII for use in treating cancer.

Embodiment XIV. The compound of Embodiment XIII, wherein the cancer is a cancer listed in Table I.

Embodiment XV. The compound of Embodiment XIII, wherein the cancer is a cancer wherein the inhibition or degradation of ER protein provides a benefit.

Embodiment XVI. Use of a Compound of the Disclosure for the manufacture of a medicament for treatment of cancer, a chronic autoimmune disorder, an inflammatory condition, a proliferative disorder, sepsis, or a viral infection.

Embodiment XVII. The use of Embodiment XVI for the treatment of cancer.

Embodiment XVIII. The use of Embodiment XVII, wherein the cancer is a cancer listed in Table I.

Embodiment XIV. The use of Embodiment XVII, wherein the cancer is breast cancer.

Embodiment XX. The use of Embodiment XVII, wherein the cancer is a cancer wherein the inhibition or degradation of ER provides a benefit.

Embodiment XXI. A method of reducing ER proteins, e.g., ERα, ERβ, or both, within a cell of a subject in need thereof, the method comprising administering to the patient a Compound of the Disclosure. In some embodiments, the ER protein is reduced by about 50% or less, e.g., 1%, about 2%, about 3%, about 4%, about 5%, about 10%, about 15%, about 20%, about 25%, about 30%, about 35%, about 40%, or about 45%. In some embodiments, the ER protein is reduced by about 51% or more, e.g., about 55%, about 60%, about 65%, about 70%, about 75%, about 80%, about 85%, about 90%, or about 95%

### Intermediates of the Disclosure

In some aspects, the disclosure provides compounds of any one of Formulae I-VIII, or a salt or solvate thereof, wherein B¹ is hydrogen or hydroxy. These compounds are collectively, or a salt or solvate thereof are collectively referred to as “Intermediates of the Disclosure.” Intermediates of the Disclosure are synthetic intermediates that can be used to make Compounds of the Disclosure.

some embodiments, Intermediates of the Disclosure are compounds of any one of Formulae I-VIII, or a salt or solvate thereof, wherein B¹ is hydrogen.

some embodiments, Intermediates of the Disclosure are compounds of any one of Formulae I-VIII, or a salt or solvate thereof, wherein B¹ is hydroxy.

In some embodiments, Intermediates of the Disclosure are selected from any one or more of the compounds of Table 2, and salts and solvates thereof.

In some embodiments, Intermediates of the Disclosure are selected from any one or more of the compounds of Table 2, and salts thereof.

In some embodiments, Intermediates of the Disclosure are selected from any one or more of the compounds of Table 2.

TABLE 2

| | | |
|---|---|---|
| 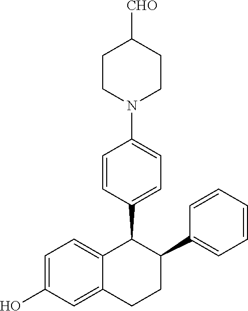 [SMILES: O=CC1CCN(c2ccc([C@@H]3c4ccc(O)cc4CC[C@@H]3c3ccccc3)cc2)CC1] | 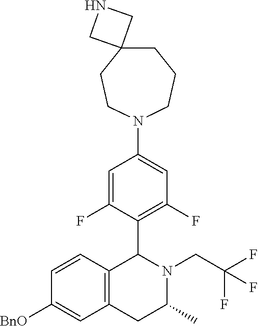 [SMILES: C[C@@H]1Cc2cc(OCc3ccccc3)ccc2C(c2c(F)cc(N3CCCC4(CC3)CNC4)cc2F)N1CC(F)(F)F] | 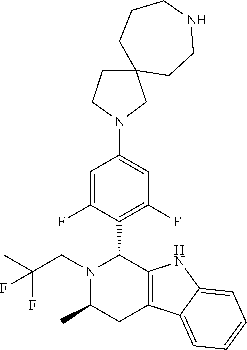 [SMILES: C[C@@H]1Cc2c([nH]c3ccccc23)[C@@H](c2c(F)cc(N3CCC4(CCCNCC4)C3)cc2F)N1CC(C)(F)F] |
| 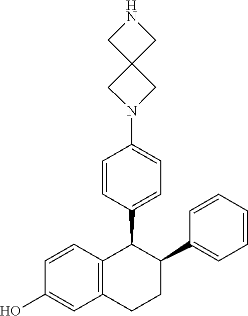 [SMILES: Oc1ccc2c(c1)CC[C@H](c1ccccc1)[C@@H]2c1ccc(N2CC3(CNC3)C2)cc1] | 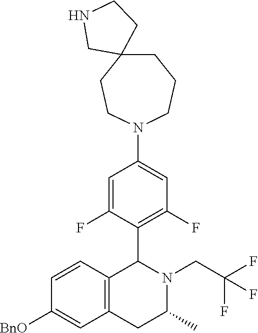 [SMILES: C[C@@H]1Cc2cc(OCc3ccccc3)ccc2C(c2c(F)cc(N3CCCC4(CCNC4)CC3)cc2F)N1CC(F)(F)F] | 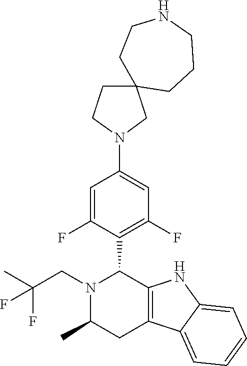 [SMILES: C[C@@H]1Cc2c([nH]c3ccccc23)[C@@H](c2c(F)cc(N3CCC4(CCCNCC4)C3)cc2F)N1CC(C)(F)F] |
| 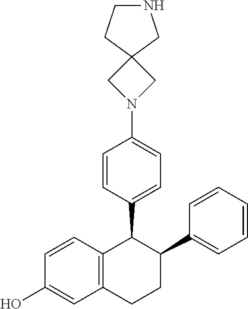 [SMILES: Oc1ccc2c(c1)CC[C@H](c1ccccc1)[C@@H]2c1ccc(N2CC3(CCNC3)C2)cc1] | 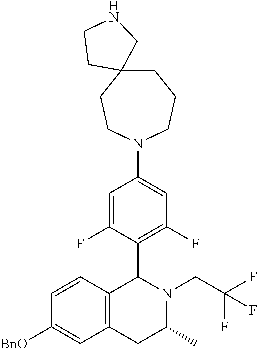 [SMILES: C[C@@H]1Cc2cc(OCc3ccccc3)ccc2C(c2c(F)cc(N3CCCC4(CCNC4)CC3)cc2F)N1CC(F)(F)F] | 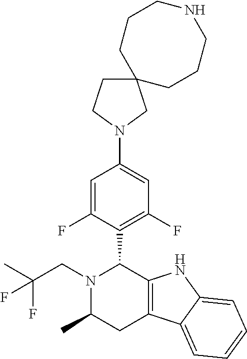 [SMILES: C[C@@H]1Cc2c([nH]c3ccccc23)[C@@H](c2c(F)cc(N3CCC4(CCCNCCC4)C3)cc2F)N1CC(C)(F)F] |
| 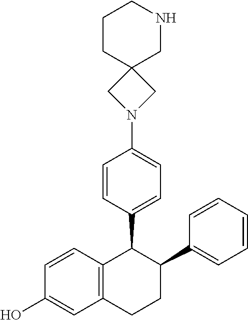 [SMILES: Oc1ccc2c(c1)CC[C@H](c1ccccc1)[C@@H]2c1ccc(N2CC3(CCCNC3)C2)cc1] | 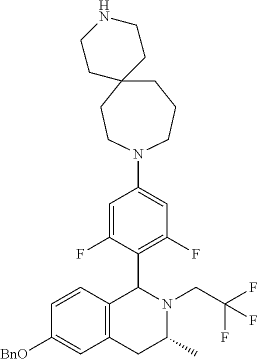 [SMILES: C[C@@H]1Cc2cc(OCc3ccccc3)ccc2C(c2c(F)cc(N3CCCC4(CCNCC4)CC3)cc2F)N1CC(F)(F)F] | 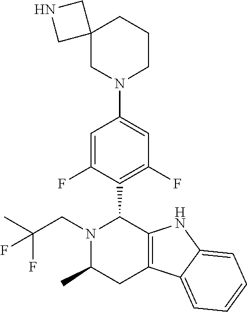 [SMILES: C[C@@H]1Cc2c([nH]c3ccccc23)[C@@H](c2c(F)cc(N3CCCC4(CNC4)C3)cc2F)N1CC(C)(F)F] |
| 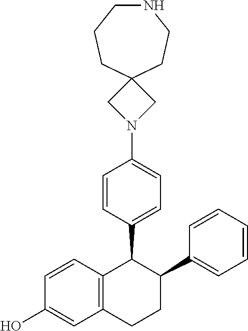 [SMILES: Oc1ccc2c(c1)CC[C@H](c1ccccc1)[C@@H]2c1ccc(N2CC3(CCCNCC3)C2)cc1] | 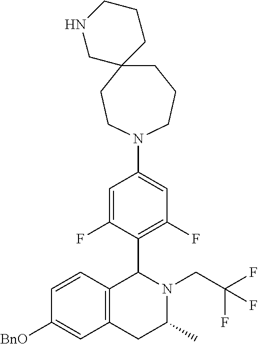 [SMILES: C[C@@H]1Cc2cc(OCc3ccccc3)ccc2C(c2c(F)cc(N3CCCC4(CCCNC4)CC3)cc2F)N1CC(F)(F)F] | 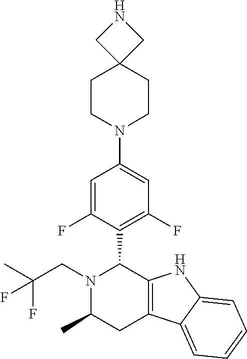 [SMILES: C[C@@H]1Cc2c([nH]c3ccccc23)[C@@H](c2c(F)cc(N3CCC4(CC3)CNC4)cc2F)N1CC(C)(F)F] |
| 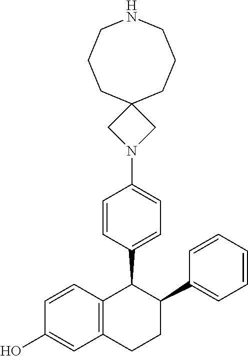 [SMILES: Oc1ccc2c(c1)CC[C@H](c1ccccc1)[C@@H]2c1ccc(N2CC3(CCCNCCC3)C2)cc1] | 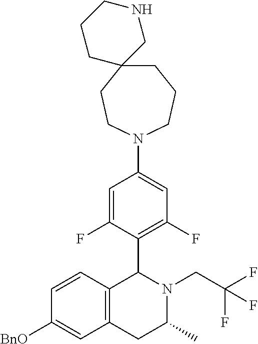 [SMILES: C[C@@H]1Cc2cc(OCc3ccccc3)ccc2C(c2c(F)cc(N3CCCC4(CCCNC4)CC3)cc2F)N1CC(F)(F)F] | 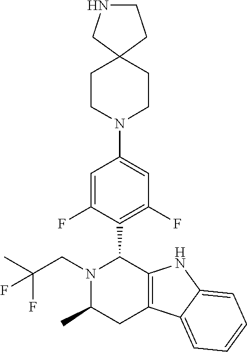 [SMILES: C[C@@H]1Cc2c([nH]c3ccccc23)[C@@H](c2c(F)cc(N3CCC4(CCNC4)CC3)cc2F)N1CC(C)(F)F] |
| 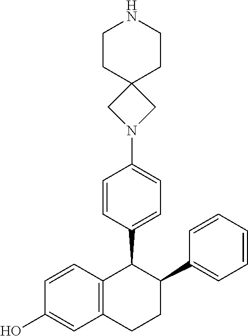 [SMILES: Oc1ccc2c(c1)CC[C@H](c1ccccc1)[C@@H]2c1ccc(N2CC3(CCNCC3)C2)cc1] | 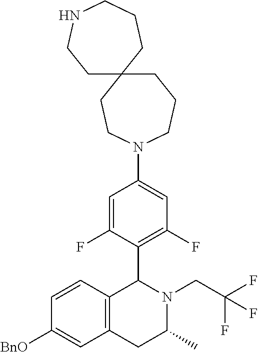 [SMILES: C[C@@H]1Cc2cc(OCc3ccccc3)ccc2C(c2c(F)cc(N3CCCC4(CCCNCC4)CC3)cc2F)N1CC(F)(F)F] | 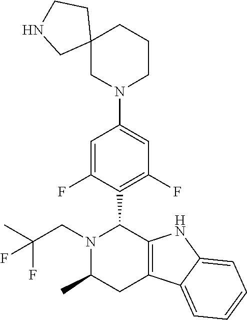 [SMILES: C[C@@H]1Cc2c([nH]c3ccccc23)[C@@H](c2c(F)cc(N3CCCC4(CCNC4)C3)cc2F)N1CC(C)(F)F] |
| 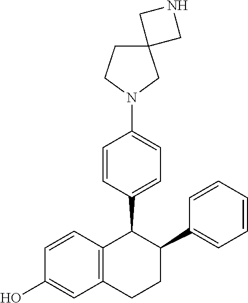 [SMILES: Oc1ccc2c(c1)CC[C@H](c1ccccc1)[C@@H]2c1ccc(N2CCC3(CNC3)C2)cc1] | 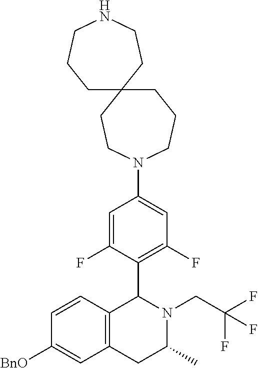 [SMILES: C[C@@H]1Cc2cc(OCc3ccccc3)ccc2C(c2c(F)cc(N3CCCC4(CCCNCC4)CC3)cc2F)N1CC(F)(F)F] | 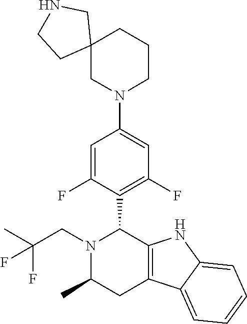 [SMILES: C[C@@H]1Cc2c([nH]c3ccccc23)[C@@H](c2c(F)cc(N3CCCC4(CCNC4)C3)cc2F)N1CC(C)(F)F] |
| 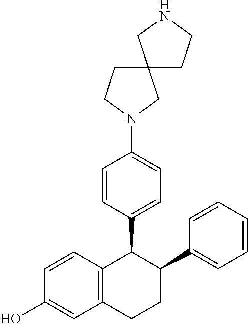 [SMILES: Oc1ccc2c(c1)CC[C@H](c1ccccc1)[C@@H]2c1ccc(N2CCC3(CCNC3)C2)cc1] | 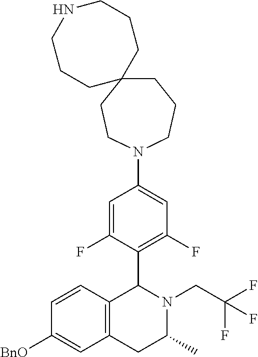 [SMILES: C[C@@H]1Cc2cc(OCc3ccccc3)ccc2C(c2c(F)cc(N3CCCC4(CCCNCCC4)CC3)cc2F)N1CC(F)(F)F] | 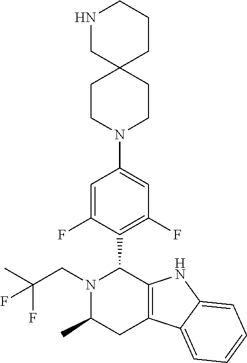 [SMILES: C[C@@H]1Cc2c([nH]c3ccccc23)[C@@H](c2c(F)cc(N3CCC4(CCCNC4)CC3)cc2F)N1CC(C)(F)F] |
| 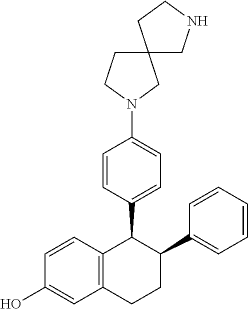 [SMILES: Oc1ccc2c(c1)CC[C@H](c1ccccc1)[C@@H]2c1ccc(N2CCC3(CCNC3)C2)cc1] | 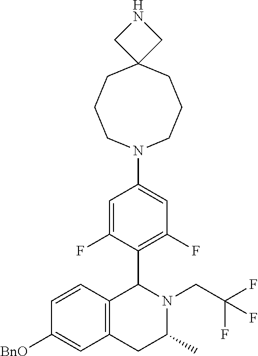 [SMILES: C[C@@H]1Cc2cc(OCc3ccccc3)ccc2C(c2c(F)cc(N3CCCC4(CCC3)CNC4)cc2F)N1CC(F)(F)F] | 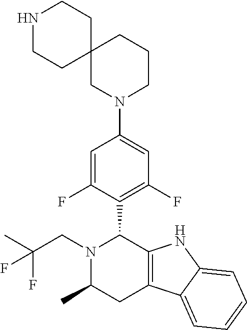 [SMILES: C[C@@H]1Cc2c([nH]c3ccccc23)[C@@H](c2c(F)cc(N3CCCC4(CCNCC4)C3)cc2F)N1CC(C)(F)F] |
| 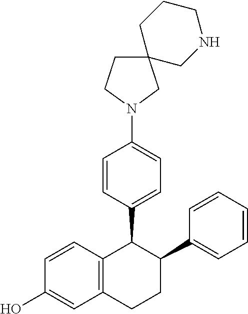 [SMILES: Oc1ccc2c(c1)CC[C@H](c1ccccc1)[C@@H]2c1ccc(N2CCC3(CCCNC3)C2)cc1] | 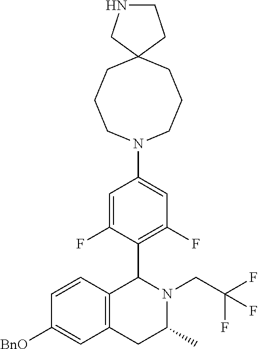 [SMILES: C[C@@H]1Cc2cc(OCc3ccccc3)ccc2C(c2c(F)cc(N3CCCC4(CCC3)CCNC4)cc2F)N1CC(F)(F)F] | 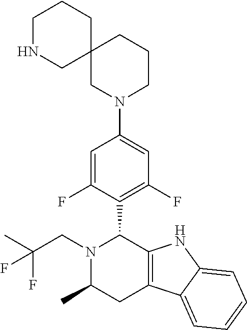 [SMILES: C[C@@H]1Cc2c([nH]c3ccccc23)[C@@H](c2c(F)cc(N3CCCC4(CCCNC4)C3)cc2F)N1CC(C)(F)F] |
| 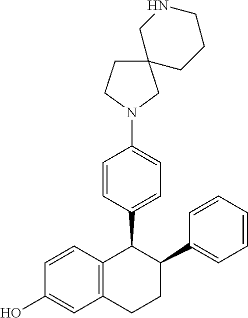 [SMILES: Oc1ccc2c(c1)CC[C@H](c1ccccc1)[C@@H]2c1ccc(N2CCC3(CCCNC3)C2)cc1] | 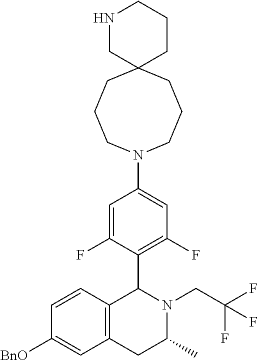 [SMILES: C[C@@H]1Cc2cc(OCc3ccccc3)ccc2C(c2c(F)cc(N3CCCC4(CCCNC4)CCC3)cc2F)N1CC(F)(F)F] | 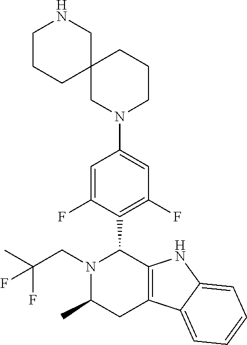 [SMILES: C[C@@H]1Cc2c([nH]c3ccccc23)[C@@H](c2c(F)cc(N3CCCC4(CCCNC4)C3)cc2F)N1CC(C)(F)F] |
| 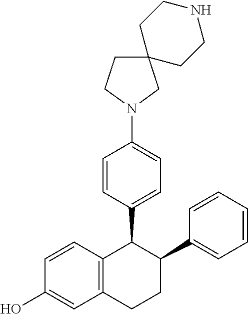 [SMILES: Oc1ccc2c(c1)CC[C@H](c1ccccc1)[C@@H]2c1ccc(N2CCC3(CCNCC3)C2)cc1] | 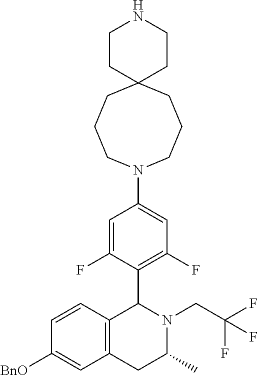 [SMILES: C[C@@H]1Cc2cc(OCc3ccccc3)ccc2C(c2c(F)cc(N3CCCC4(CCC3)CCNCC4)cc2F)N1CC(F)(F)F] | 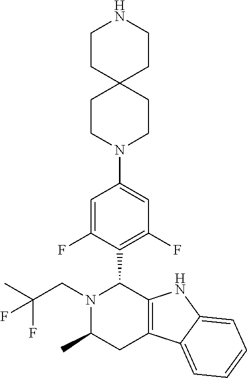 [SMILES: C[C@@H]1Cc2c([nH]c3ccccc23)[C@@H](c2c(F)cc(N3CCC4(CCNCC4)CC3)cc2F)N1CC(C)(F)F] |
| 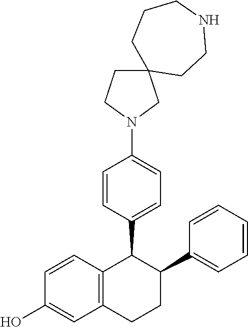 [SMILES: Oc1ccc2c(c1)CC[C@H](c1ccccc1)[C@@H]2c1ccc(N2CCC3(CCCNCC3)C2)cc1] | 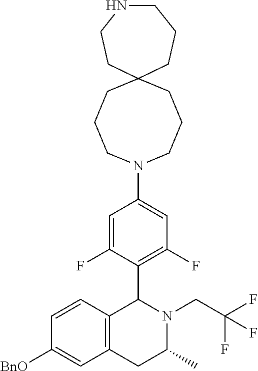 [SMILES: C[C@@H]1Cc2cc(OCc3ccccc3)ccc2C(c2c(F)cc(N3CCCC4(CCCNCC4)CCC3)cc2F)N1CC(F)(F)F] | 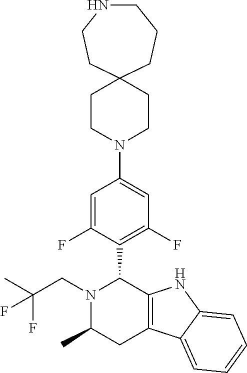 [SMILES: C[C@@H]1Cc2c([nH]c3ccccc23)[C@@H](c2c(F)cc(N3CCC4(CCCNCC4)CC3)cc2F)N1CC(C)(F)F] |
| 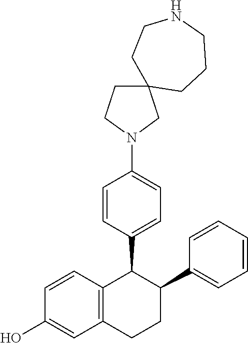 [SMILES: Oc1ccc2c(c1)CC[C@H](c1ccccc1)[C@@H]2c1ccc(N2CCC3(CCCNCC3)C2)cc1] | 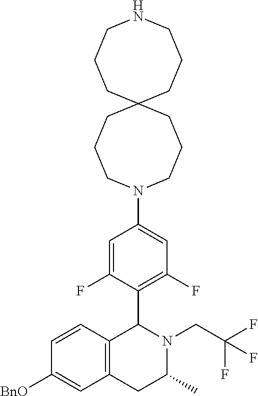 [SMILES: C[C@@H]1Cc2cc(OCc3ccccc3)ccc2C(c2c(F)cc(N3CCCC4(CCCNCCC4)CCC3)cc2F)N1CC(F)(F)F] | 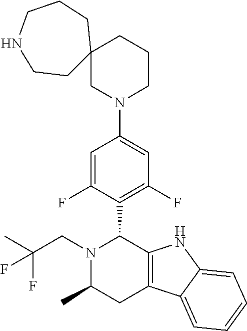 [SMILES: C[C@@H]1Cc2c([nH]c3ccccc23)[C@@H](c2c(F)cc(N3CCCC4(CCCNCC4)C3)cc2F)N1CC(C)(F)F] |
| 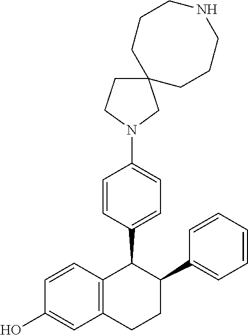 [SMILES: Oc1ccc2c(c1)CC[C@H](c1ccccc1)[C@@H]2c1ccc(N2CCC3(CCCNCCC3)C2)cc1] | 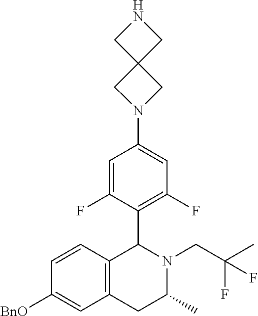 [SMILES: C[C@@H]1Cc2cc(OCc3ccccc3)ccc2C(c2c(F)cc(N3CC4(CNC4)C3)cc2F)N1CC(C)(F)F] | 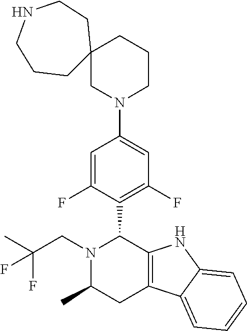 [SMILES: C[C@@H]1Cc2c([nH]c3ccccc23)[C@@H](c2c(F)cc(N3CCCC4(CCCNCC4)C3)cc2F)N1CC(C)(F)F] |
| 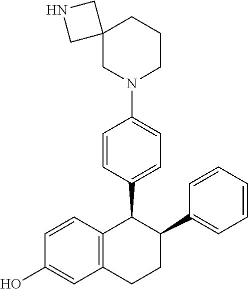 [SMILES: Oc1ccc2c(c1)CC[C@H](c1ccccc1)[C@@H]2c1ccc(N2CCCC3(CNC3)C2)cc1] | 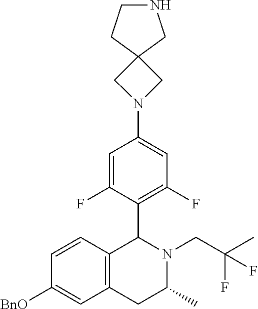 [SMILES: C[C@@H]1Cc2cc(OCc3ccccc3)ccc2C(c2c(F)cc(N3CC4(CCNC4)C3)cc2F)N1CC(C)(F)F] | 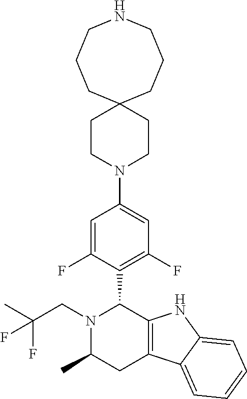 [SMILES: C[C@@H]1Cc2c([nH]c3ccccc23)[C@@H](c2c(F)cc(N3CCC4(CCCNCCC4)CC3)cc2F)N1CC(C)(F)F] |
| 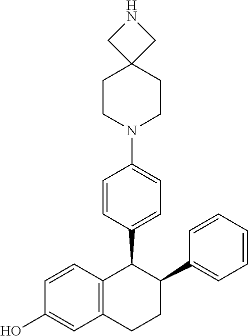 [SMILES: Oc1ccc2c(c1)CC[C@H](c1ccccc1)[C@@H]2c1ccc(N2CCC3(CC2)CNC3)cc1] | 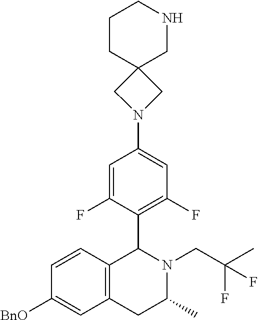 [SMILES: C[C@@H]1Cc2cc(OCc3ccccc3)ccc2C(c2c(F)cc(N3CC4(CCCNC4)C3)cc2F)N1CC(C)(F)F] | 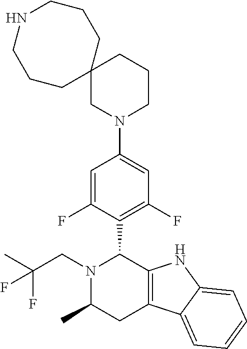 [SMILES: C[C@@H]1Cc2c([nH]c3ccccc23)[C@@H](c2c(F)cc(N3CCCC4(CCCNCCC4)C3)cc2F)N1CC(C)(F)F] |
| 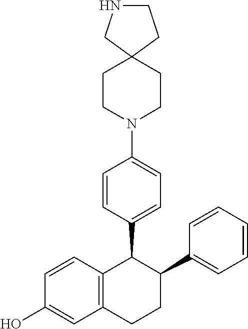 [SMILES: Oc1ccc2c(c1)CC[C@H](c1ccccc1)[C@@H]2c1ccc(N2CCC3(CCNC3)CC2)cc1] | 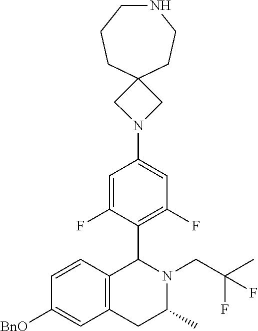 [SMILES: C[C@@H]1Cc2cc(OCc3ccccc3)ccc2C(c2c(F)cc(N3CC4(CCCNCC4)C3)cc2F)N1CC(C)(F)F] | 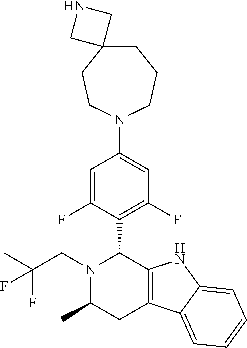 [SMILES: C[C@@H]1Cc2c([nH]c3ccccc23)[C@@H](c2c(F)cc(N3CCCC4(CC3)CNC4)cc2F)N1CC(C)(F)F] |
| 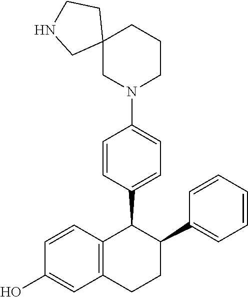 [SMILES: Oc1ccc2c(c1)CC[C@H](c1ccccc1)[C@@H]2c1ccc(N2CCCC3(CCNC3)C2)cc1] | 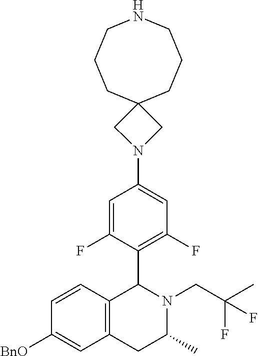 [SMILES: C[C@@H]1Cc2cc(OCc3ccccc3)ccc2C(c2c(F)cc(N3CC4(CCCNCCC4)C3)cc2F)N1CC(C)(F)F] | 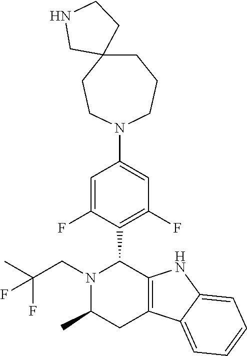 [SMILES: C[C@@H]1Cc2c([nH]c3ccccc23)[C@@H](c2c(F)cc(N3CCCC4(CCNC4)CC3)cc2F)N1CC(C)(F)F] |
| 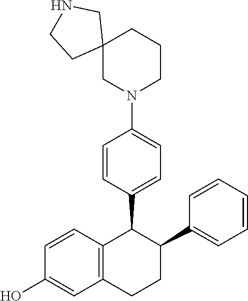 [SMILES: Oc1ccc2c(c1)CC[C@H](c1ccccc1)[C@@H]2c1ccc(N2CCCC3(CCNC3)C2)cc1] | 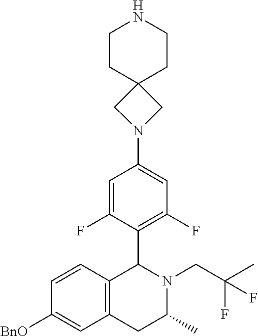 [SMILES: C[C@@H]1Cc2cc(OCc3ccccc3)ccc2C(c2c(F)cc(N3CC4(CCNCC4)C3)cc2F)N1CC(C)(F)F] | 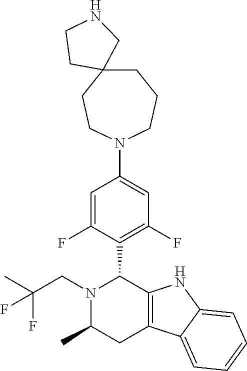 [SMILES: C[C@@H]1Cc2c([nH]c3ccccc23)[C@@H](c2c(F)cc(N3CCCC4(CCNC4)CC3)cc2F)N1CC(C)(F)F] |
| 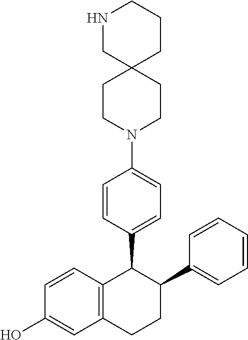 [SMILES: Oc1ccc2c(c1)CC[C@H](c1ccccc1)[C@@H]2c1ccc(N2CCC3(CCCNC3)CC2)cc1] | 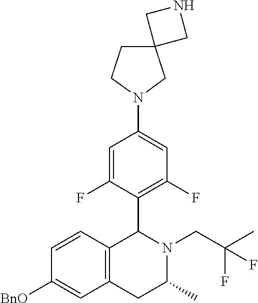 [SMILES: C[C@@H]1Cc2cc(OCc3ccccc3)ccc2C(c2c(F)cc(N3CCC4(CNC4)C3)cc2F)N1CC(C)(F)F] | 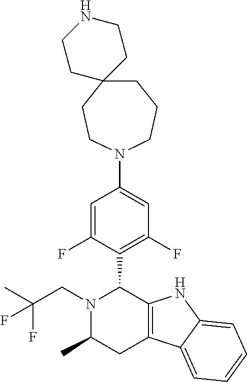 [SMILES: C[C@@H]1Cc2c([nH]c3ccccc23)[C@@H](c2c(F)cc(N3CCCC4(CCNCC4)CC3)cc2F)N1CC(C)(F)F] |
| 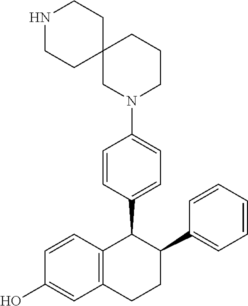 [SMILES: Oc1ccc2c(c1)CC[C@H](c1ccccc1)[C@@H]2c1ccc(N2CCCC3(CCNCC3)C2)cc1] | 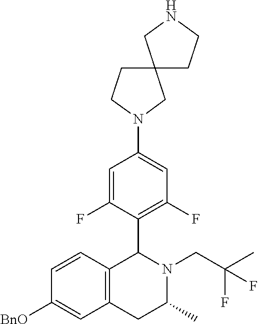 [SMILES: C[C@@H]1Cc2cc(OCc3ccccc3)ccc2C(c2c(F)cc(N3CCC4(CCNC4)C3)cc2F)N1CC(C)(F)F] | 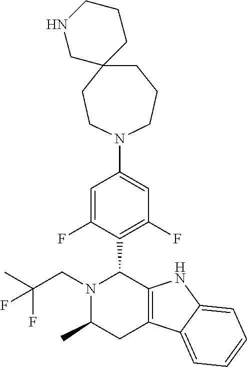 [SMILES: C[C@@H]1Cc2c([nH]c3ccccc23)[C@@H](c2c(F)cc(N3CCCC4(CCCNC4)CC3)cc2F)N1CC(C)(F)F] |
| 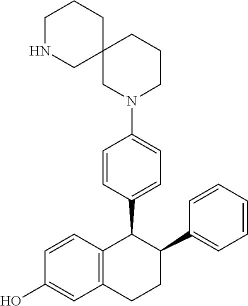 [SMILES: Oc1ccc2c(c1)CC[C@H](c1ccccc1)[C@@H]2c1ccc(N2CCCC3(CCCNC3)C2)cc1] | 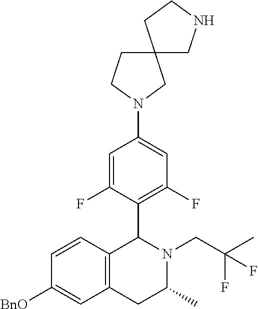 [SMILES: C[C@@H]1Cc2cc(OCc3ccccc3)ccc2C(c2c(F)cc(N3CCC4(CCNC4)C3)cc2F)N1CC(C)(F)F] | 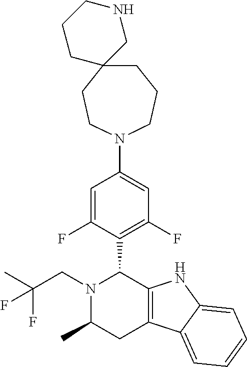 [SMILES: C[C@@H]1Cc2c([nH]c3ccccc23)[C@@H](c2c(F)cc(N3CCCC4(CCCNC4)CC3)cc2F)N1CC(C)(F)F] |
| 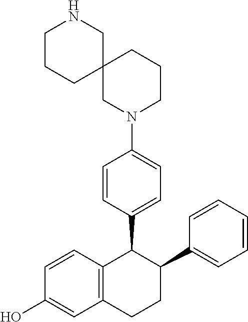 [SMILES: Oc1ccc2c(c1)CC[C@H](c1ccccc1)[C@@H]2c1ccc(N2CCCC3(CCCNC3)C2)cc1] | 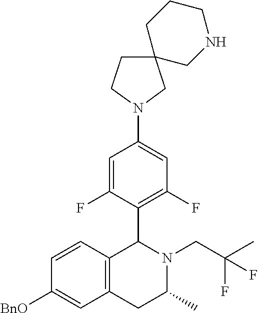 [SMILES: C[C@@H]1Cc2cc(OCc3ccccc3)ccc2C(c2c(F)cc(N3CCC4(CCCNC4)C3)cc2F)N1CC(C)(F)F] | 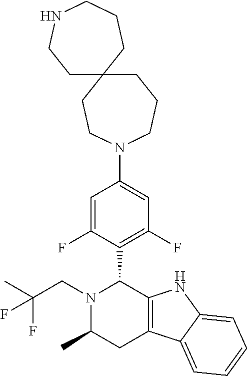 [SMILES: C[C@@H]1Cc2c([nH]c3ccccc23)[C@@H](c2c(F)cc(N3CCCC4(CCCNCC4)CC3)cc2F)N1CC(C)(F)F] |
| 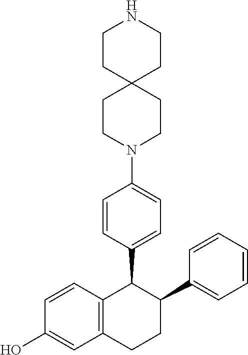 [SMILES: Oc1ccc2c(c1)CC[C@H](c1ccccc1)[C@@H]2c1ccc(N2CCC3(CCNCC3)CC2)cc1] | 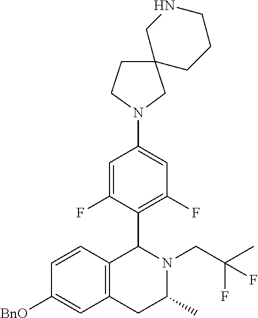 [SMILES: C[C@@H]1Cc2cc(OCc3ccccc3)ccc2C(c2c(F)cc(N3CCC4(CCCNC4)C3)cc2F)N1CC(C)(F)F] | 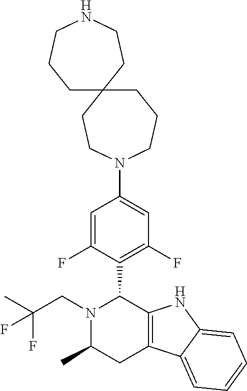 [SMILES: C[C@@H]1Cc2c([nH]c3ccccc23)[C@@H](c2c(F)cc(N3CCCC4(CCCNCC4)CC3)cc2F)N1CC(C)(F)F] |
| 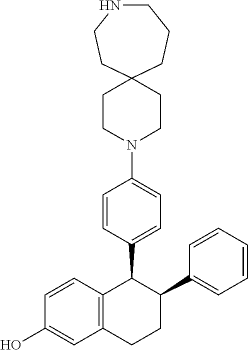 [SMILES: Oc1ccc2c(c1)CC[C@H](c1ccccc1)[C@@H]2c1ccc(N2CCC3(CCCNCC3)CC2)cc1] | 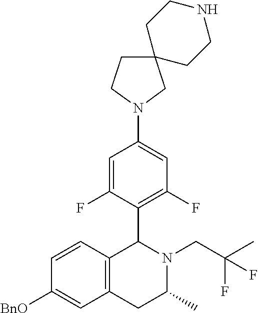 [SMILES: C[C@@H]1Cc2cc(OCc3ccccc3)ccc2C(c2c(F)cc(N3CCC4(CCNCC4)C3)cc2F)N1CC(C)(F)F] | 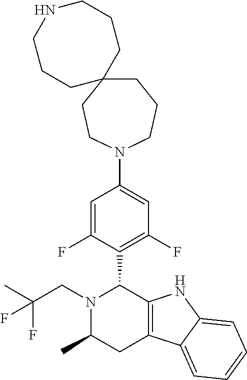 [SMILES: C[C@@H]1Cc2c([nH]c3ccccc23)[C@@H](c2c(F)cc(N3CCCC4(CCCNCCC4)CC3)cc2F)N1CC(C)(F)F] |
| 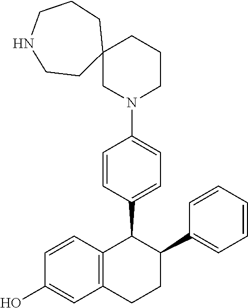 [SMILES: Oc1ccc2c(c1)CC[C@H](c1ccccc1)[C@@H]2c1ccc(N2CCCC3(CCCNCC3)C2)cc1] | 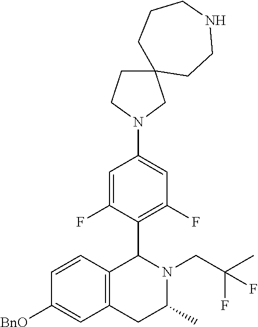 [SMILES: C[C@@H]1Cc2cc(OCc3ccccc3)ccc2C(c2c(F)cc(N3CCC4(CCCNCC4)C3)cc2F)N1CC(C)(F)F] | 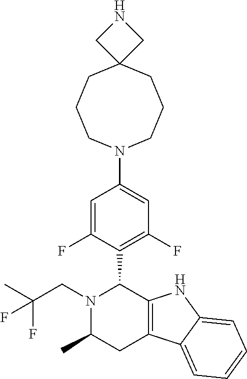 [SMILES: C[C@@H]1Cc2c([nH]c3ccccc23)[C@@H](c2c(F)cc(N3CCCC4(CCC3)CNC4)cc2F)N1CC(C)(F)F] |
| 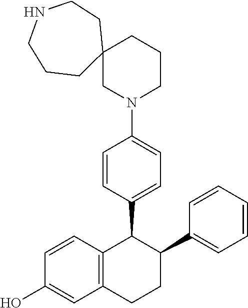 [SMILES: Oc1ccc2c(c1)CC[C@H](c1ccccc1)[C@@H]2c1ccc(N2CCCC3(CCCNCC3)C2)cc1] | 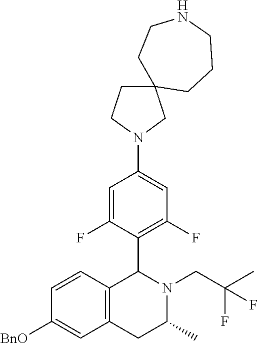 [SMILES: C[C@@H]1Cc2cc(OCc3ccccc3)ccc2C(c2c(F)cc(N3CCC4(CCCNCC4)C3)cc2F)N1CC(C)(F)F] | 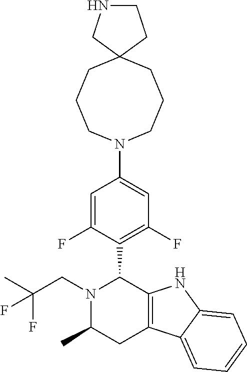 [SMILES: C[C@@H]1Cc2c([nH]c3ccccc23)[C@@H](c2c(F)cc(N3CCCC4(CCC3)CCNC4)cc2F)N1CC(C)(F)F] |
| 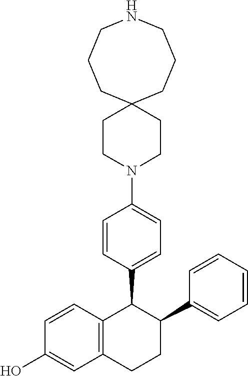 [SMILES: Oc1ccc2c(c1)CC[C@H](c1ccccc1)[C@@H]2c1ccc(N2CCC3(CCCNCCC3)CC2)cc1] | 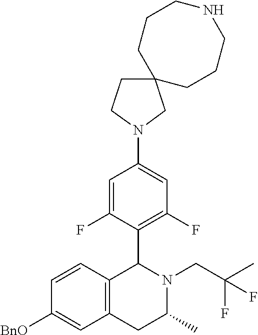 [SMILES: C[C@@H]1Cc2cc(OCc3ccccc3)ccc2C(c2c(F)cc(N3CCC4(CCCNCCC4)C3)cc2F)N1CC(C)(F)F] | 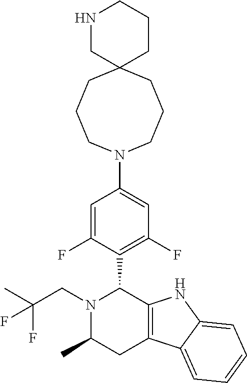 [SMILES: C[C@@H]1Cc2c([nH]c3ccccc23)[C@@H](c2c(F)cc(N3CCCC4(CCCNC4)CCC3)cc2F)N1CC(C)(F)F] |
| 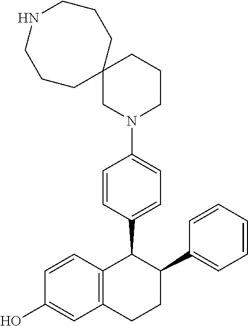 [SMILES: Oc1ccc2c(c1)CC[C@H](c1ccccc1)[C@@H]2c1ccc(N2CCCC3(CCCNCCC3)C2)cc1] | 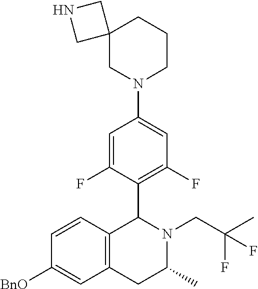 [SMILES: C[C@@H]1Cc2cc(OCc3ccccc3)ccc2C(c2c(F)cc(N3CCCC4(CNC4)C3)cc2F)N1CC(C)(F)F] | 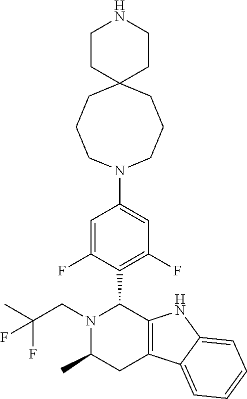 [SMILES: C[C@@H]1Cc2c([nH]c3ccccc23)[C@@H](c2c(F)cc(N3CCCC4(CCC3)CCNCC4)cc2F)N1CC(C)(F)F] |
| 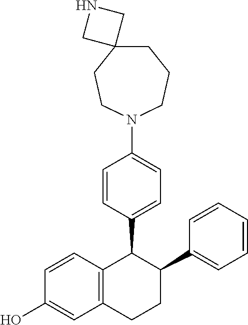 [SMILES: Oc1ccc2c(c1)CC[C@H](c1ccccc1)[C@@H]2c1ccc(N2CCCC3(CC2)CNC3)cc1] | 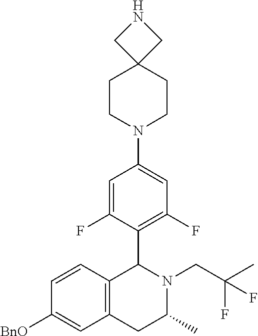 [SMILES: C[C@@H]1Cc2cc(OCc3ccccc3)ccc2C(c2c(F)cc(N3CCC4(CC3)CNC4)cc2F)N1CC(C)(F)F] | 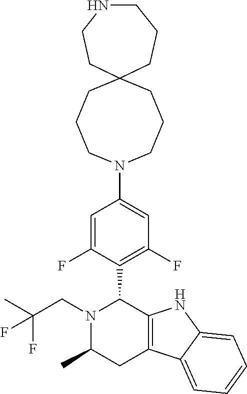 [SMILES: C[C@@H]1Cc2c([nH]c3ccccc23)[C@@H](c2c(F)cc(N3CCCC4(CCCNCC4)CCC3)cc2F)N1CC(C)(F)F] |
| 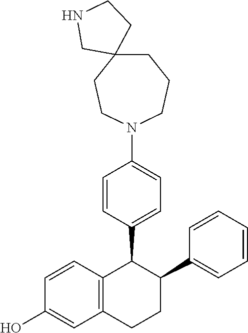 [SMILES: Oc1ccc2c(c1)CC[C@H](c1ccccc1)[C@@H]2c1ccc(N2CCCC3(CCNC3)CC2)cc1] | 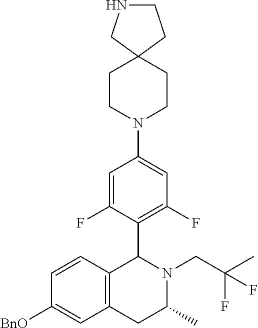 [SMILES: C[C@@H]1Cc2cc(OCc3ccccc3)ccc2C(c2c(F)cc(N3CCC4(CCNC4)CC3)cc2F)N1CC(C)(F)F] | 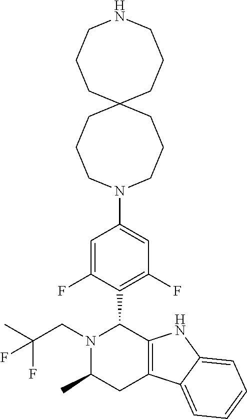 [SMILES: C[C@@H]1Cc2c([nH]c3ccccc23)[C@@H](c2c(F)cc(N3CCCC4(CCCNCCC4)CCC3)cc2F)N1CC(C)(F)F] |
| 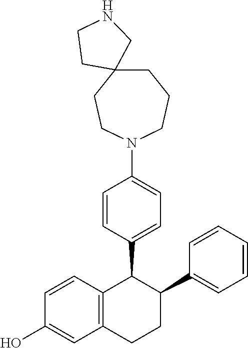 [SMILES: Oc1ccc2c(c1)CC[C@H](c1ccccc1)[C@@H]2c1ccc(N2CCCC3(CCNC3)CC2)cc1] | 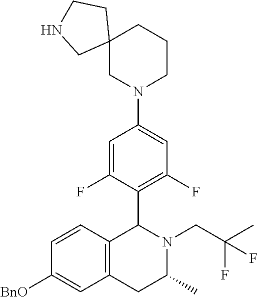 [SMILES: C[C@@H]1Cc2cc(OCc3ccccc3)ccc2C(c2c(F)cc(N3CCCC4(CCNC4)C3)cc2F)N1CC(C)(F)F] | 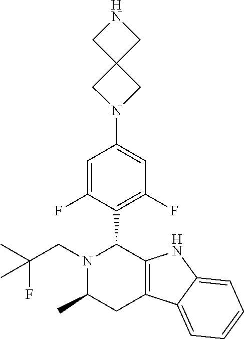 [SMILES: C[C@@H]1Cc2c([nH]c3ccccc23)[C@@H](c2c(F)cc(N3CC4(CNC4)C3)cc2F)N1CC(C)(C)F] |
| 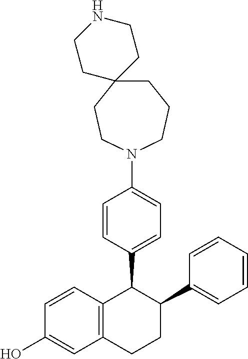 [SMILES: Oc1ccc2c(c1)CC[C@H](c1ccccc1)[C@@H]2c1ccc(N2CCCC3(CCNCC3)CC2)cc1] | 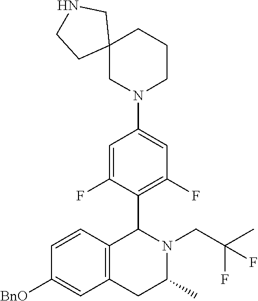 [SMILES: C[C@@H]1Cc2cc(OCc3ccccc3)ccc2C(c2c(F)cc(N3CCCC4(CCNC4)C3)cc2F)N1CC(C)(F)F] | 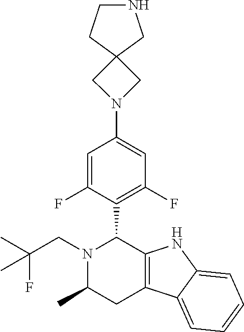 [SMILES: C[C@@H]1Cc2c([nH]c3ccccc23)[C@@H](c2c(F)cc(N3CC4(CCNC4)C3)cc2F)N1CC(C)(C)F] |
| 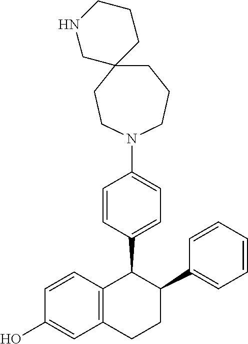 [SMILES: Oc1ccc2c(c1)CC[C@H](c1ccccc1)[C@@H]2c1ccc(N2CCCC3(CCCNC3)CC2)cc1] | 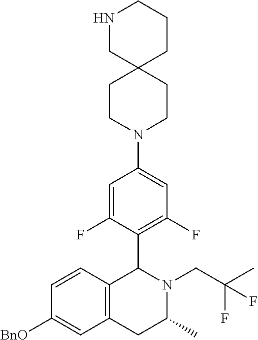 [SMILES: C[C@@H]1Cc2cc(OCc3ccccc3)ccc2C(c2c(F)cc(N3CCC4(CCCNC4)CC3)cc2F)N1CC(C)(F)F] | 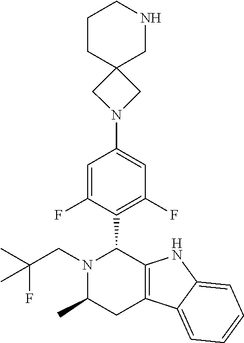 [SMILES: C[C@@H]1Cc2c([nH]c3ccccc23)[C@@H](c2c(F)cc(N3CC4(CCCNC4)C3)cc2F)N1CC(C)(C)F] |
| 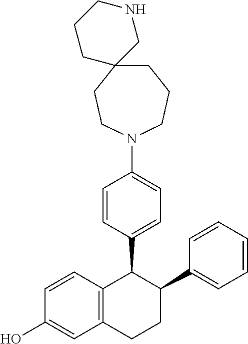 [SMILES: Oc1ccc2c(c1)CC[C@H](c1ccccc1)[C@@H]2c1ccc(N2CCCC3(CCCNC3)CC2)cc1] | 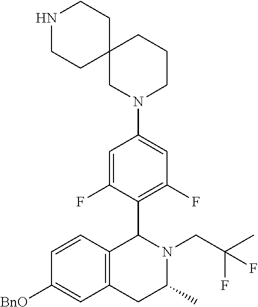 [SMILES: C[C@@H]1Cc2cc(OCc3ccccc3)ccc2C(c2c(F)cc(N3CCCC4(CCNCC4)C3)cc2F)N1CC(C)(F)F] | 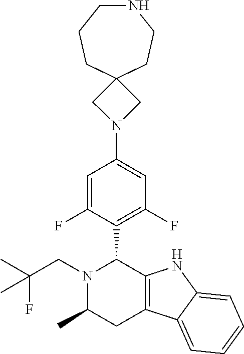 [SMILES: C[C@@H]1Cc2c([nH]c3ccccc23)[C@@H](c2c(F)cc(N3CC4(CCCNCC4)C3)cc2F)N1CC(C)(C)F] |
| 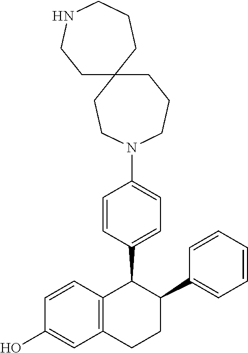 [SMILES: Oc1ccc2c(c1)CC[C@H](c1ccccc1)[C@@H]2c1ccc(N2CCCC3(CCCNCC3)CC2)cc1] | 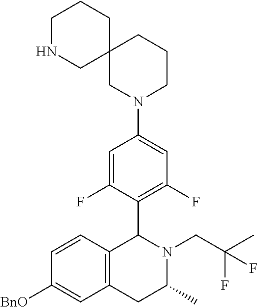 [SMILES: C[C@@H]1Cc2cc(OCc3ccccc3)ccc2C(c2c(F)cc(N3CCCC4(CCCNC4)C3)cc2F)N1CC(C)(F)F] | 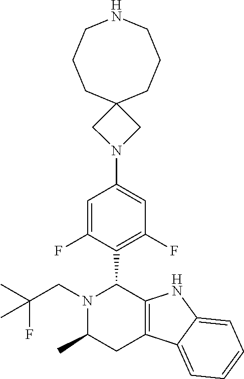 [SMILES: C[C@@H]1Cc2c([nH]c3ccccc23)[C@@H](c2c(F)cc(N3CC4(CCCNCCC4)C3)cc2F)N1CC(C)(C)F] |
| 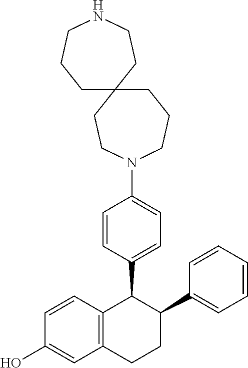 [SMILES: Oc1ccc2c(c1)CC[C@H](c1ccccc1)[C@@H]2c1ccc(N2CCCC3(CCCNCC3)CC2)cc1] | 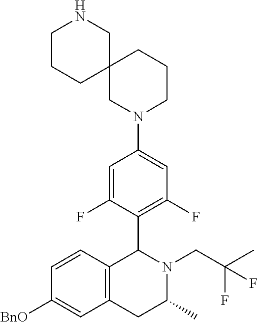 [SMILES: C[C@@H]1Cc2cc(OCc3ccccc3)ccc2C(c2c(F)cc(N3CCCC4(CCCNC4)C3)cc2F)N1CC(C)(F)F] | 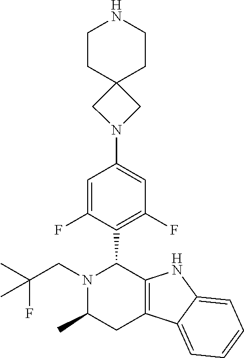 [SMILES: C[C@@H]1Cc2c([nH]c3ccccc23)[C@@H](c2c(F)cc(N3CC4(CCNCC4)C3)cc2F)N1CC(C)(C)F] |
| 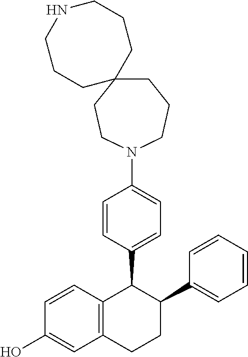 [SMILES: Oc1ccc2c(c1)CC[C@H](c1ccccc1)[C@@H]2c1ccc(N2CCCC3(CCCNCCC3)CC2)cc1] | 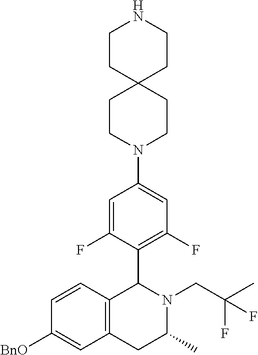 [SMILES: C[C@@H]1Cc2cc(OCc3ccccc3)ccc2C(c2c(F)cc(N3CCC4(CCNCC4)CC3)cc2F)N1CC(C)(F)F] | 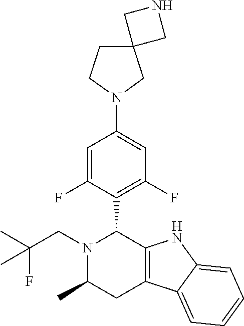 [SMILES: C[C@@H]1Cc2c([nH]c3ccccc23)[C@@H](c2c(F)cc(N3CCC4(CNC4)C3)cc2F)N1CC(C)(C)F] |
| 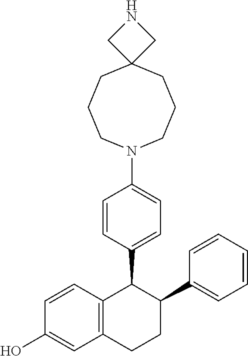 [SMILES: Oc1ccc2c(c1)CC[C@H](c1ccccc1)[C@@H]2c1ccc(N2CCCC3(CCC2)CNC3)cc1] | 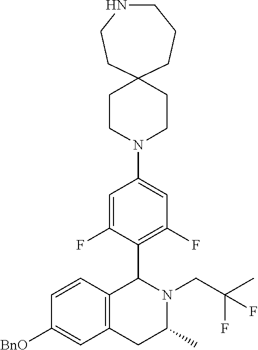 [SMILES: C[C@@H]1Cc2cc(OCc3ccccc3)ccc2C(c2c(F)cc(N3CCC4(CCCNCC4)CC3)cc2F)N1CC(C)(F)F] | 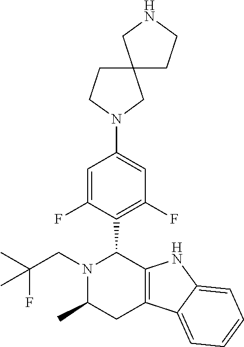 [SMILES: C[C@@H]1Cc2c([nH]c3ccccc23)[C@@H](c2c(F)cc(N3CCC4(CCNC4)C3)cc2F)N1CC(C)(C)F] |
| 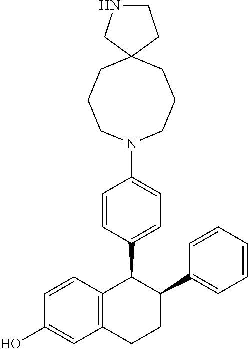 [SMILES: Oc1ccc2c(c1)CC[C@H](c1ccccc1)[C@@H]2c1ccc(N2CCCC3(CCC2)CCNC3)cc1] | 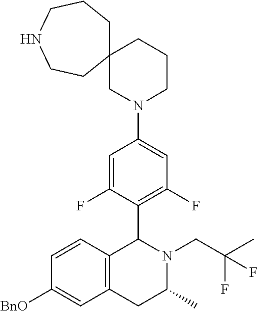 [SMILES: C[C@@H]1Cc2cc(OCc3ccccc3)ccc2C(c2c(F)cc(N3CCCC4(CCCNCC4)C3)cc2F)N1CC(C)(F)F] | 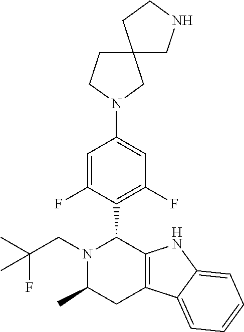 [SMILES: C[C@@H]1Cc2c([nH]c3ccccc23)[C@@H](c2c(F)cc(N3CCC4(CCNC4)C3)cc2F)N1CC(C)(C)F] |
| 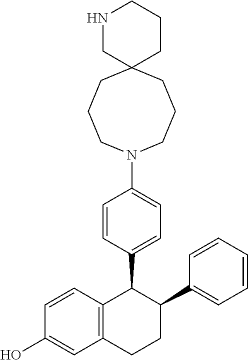 [SMILES: Oc1ccc2c(c1)CC[C@H](c1ccccc1)[C@@H]2c1ccc(N2CCCC3(CCCNC3)CCC2)cc1] | 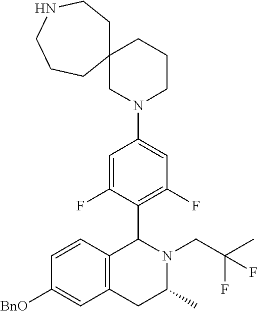 [SMILES: C[C@@H]1Cc2cc(OCc3ccccc3)ccc2C(c2c(F)cc(N3CCCC4(CCCNCC4)C3)cc2F)N1CC(C)(F)F] | 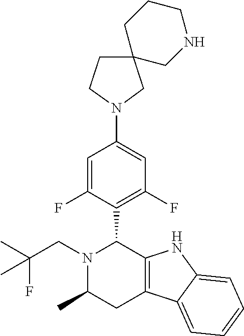 [SMILES: C[C@@H]1Cc2c([nH]c3ccccc23)[C@@H](c2c(F)cc(N3CCC4(CCCNC4)C3)cc2F)N1CC(C)(C)F] |
| 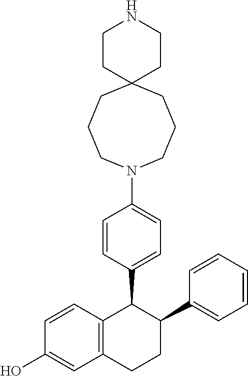 [SMILES: Oc1ccc2c(c1)CC[C@H](c1ccccc1)[C@@H]2c1ccc(N2CCCC3(CCC2)CCNCC3)cc1] | 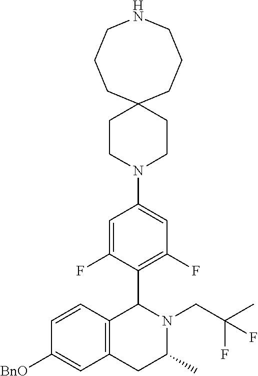 [SMILES: C[C@@H]1Cc2cc(OCc3ccccc3)ccc2C(c2c(F)cc(N3CCC4(CCCNCCC4)CC3)cc2F)N1CC(C)(F)F] | 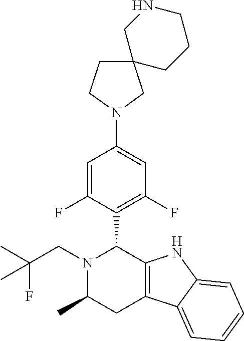 [SMILES: C[C@@H]1Cc2c([nH]c3ccccc23)[C@@H](c2c(F)cc(N3CCC4(CCCNC4)C3)cc2F)N1CC(C)(C)F] |
| 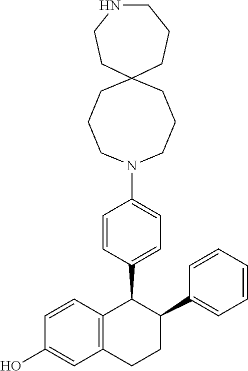 [SMILES: Oc1ccc2c(c1)CC[C@H](c1ccccc1)[C@@H]2c1ccc(N2CCCC3(CCCNCC3)CCC2)cc1] | 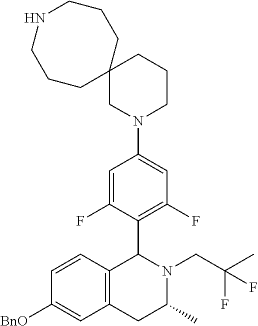 [SMILES: C[C@@H]1Cc2cc(OCc3ccccc3)ccc2C(c2c(F)cc(N3CCCC4(CCCNCCC4)C3)cc2F)N1CC(C)(F)F] | 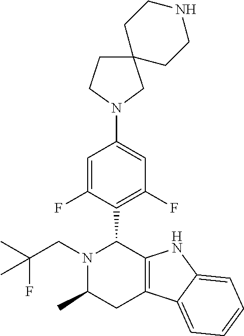 [SMILES: C[C@@H]1Cc2c([nH]c3ccccc23)[C@@H](c2c(F)cc(N3CCC4(CCNCC4)C3)cc2F)N1CC(C)(C)F] |
| 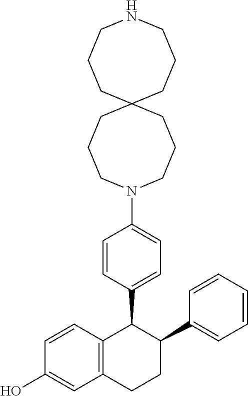 [SMILES: Oc1ccc2c(c1)CC[C@H](c1ccccc1)[C@@H]2c1ccc(N2CCCC3(CCCNCCC3)CCC2)cc1] | 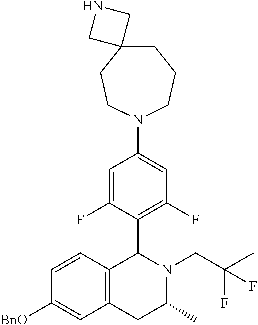 [SMILES: C[C@@H]1Cc2cc(OCc3ccccc3)ccc2C(c2c(F)cc(N3CCCC4(CC3)CNC4)cc2F)N1CC(C)(F)F] | 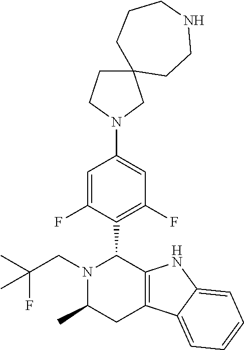 [SMILES: C[C@@H]1Cc2c([nH]c3ccccc23)[C@@H](c2c(F)cc(N3CCC4(CCCNCC4)C3)cc2F)N1CC(C)(C)F] |
| 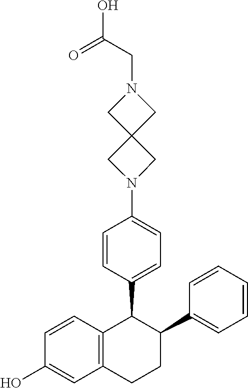 [SMILES: O=C(O)CN1CC2(C1)CN(c1ccc([C@@H]3c4ccc(O)cc4CC[C@@H]3c3ccccc3)cc1)C2] | 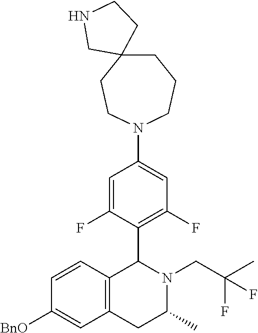 [SMILES: C[C@@H]1Cc2cc(OCc3ccccc3)ccc2C(c2c(F)cc(N3CCCC4(CCNC4)CC3)cc2F)N1CC(C)(F)F] | 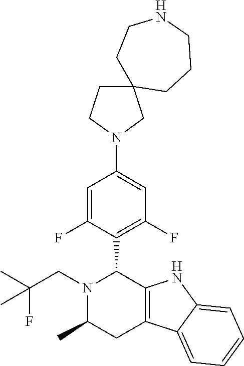 [SMILES: C[C@@H]1Cc2c([nH]c3ccccc23)[C@@H](c2c(F)cc(N3CCC4(CCCNCC4)C3)cc2F)N1CC(C)(C)F] |
| 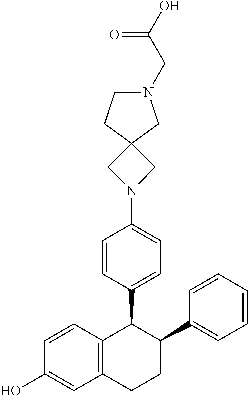 [SMILES: O=C(O)CN1CCC2(C1)CN(c1ccc([C@@H]3c4ccc(O)cc4CC[C@@H]3c3ccccc3)cc1)C2] | 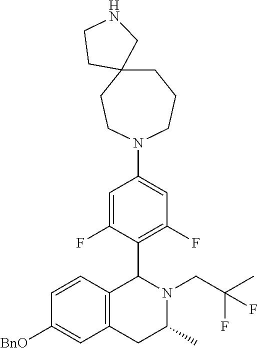 [SMILES: C[C@@H]1Cc2cc(OCc3ccccc3)ccc2C(c2c(F)cc(N3CCCC4(CCNC4)CC3)cc2F)N1CC(C)(F)F] | 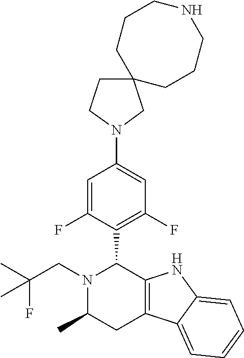 [SMILES: C[C@@H]1Cc2c([nH]c3ccccc23)[C@@H](c2c(F)cc(N3CCC4(CCCNCCC4)C3)cc2F)N1CC(C)(C)F] |
| 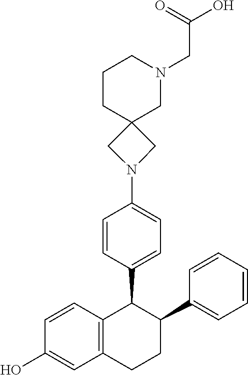 [SMILES: O=C(O)CN1CCCC2(C1)CN(c1ccc([C@@H]3c4ccc(O)cc4CC[C@@H]3c3ccccc3)cc1)C2] | 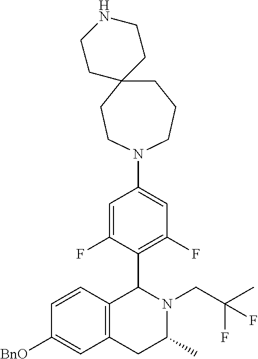 [SMILES: C[C@@H]1Cc2cc(OCc3ccccc3)ccc2C(c2c(F)cc(N3CCCC4(CCNCC4)CC3)cc2F)N1CC(C)(F)F] | 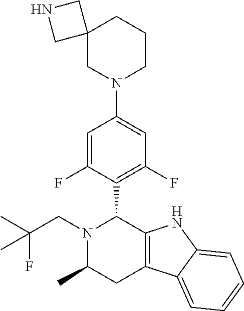 [SMILES: C[C@@H]1Cc2c([nH]c3ccccc23)[C@@H](c2c(F)cc(N3CCCC4(CNC4)C3)cc2F)N1CC(C)(C)F] |
| 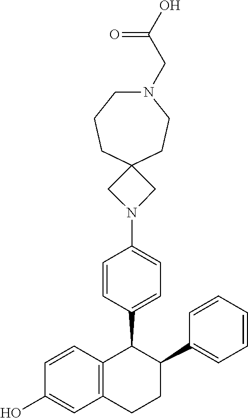 [SMILES: O=C(O)CN1CCCC2(CC1)CN(c1ccc([C@@H]3c4ccc(O)cc4CC[C@@H]3c3ccccc3)cc1)C2] | 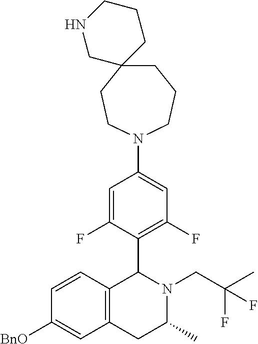 [SMILES: C[C@@H]1Cc2cc(OCc3ccccc3)ccc2C(c2c(F)cc(N3CCCC4(CCCNC4)CC3)cc2F)N1CC(C)(F)F] | 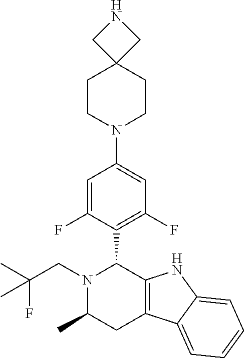 [SMILES: C[C@@H]1Cc2c([nH]c3ccccc23)[C@@H](c2c(F)cc(N3CCC4(CC3)CNC4)cc2F)N1CC(C)(C)F] |
| 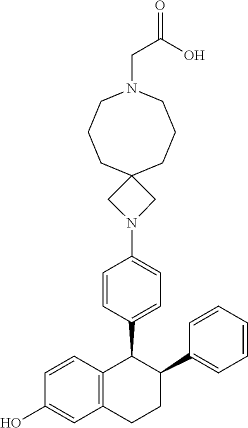 [SMILES: O=C(O)CN1CCCC2(CCC1)CN(c1ccc([C@@H]3c4ccc(O)cc4CC[C@@H]3c3ccccc3)cc1)C2] | 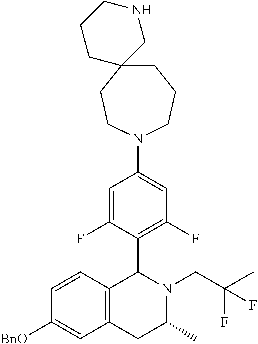 [SMILES: C[C@@H]1Cc2cc(OCc3ccccc3)ccc2C(c2c(F)cc(N3CCCC4(CCCNC4)CC3)cc2F)N1CC(C)(F)F] | 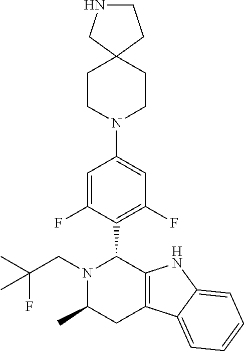 [SMILES: C[C@@H]1Cc2c([nH]c3ccccc23)[C@@H](c2c(F)cc(N3CCC4(CCNC4)CC3)cc2F)N1CC(C)(C)F] |
| 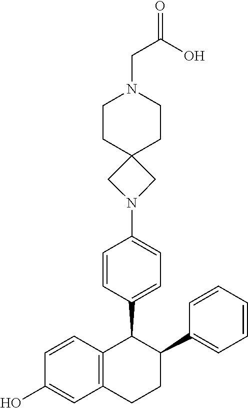 [SMILES: O=C(O)CN1CCC2(CC1)CN(c1ccc([C@@H]3c4ccc(O)cc4CC[C@@H]3c3ccccc3)cc1)C2] | 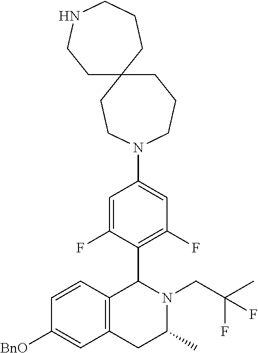 [SMILES: C[C@@H]1Cc2cc(OCc3ccccc3)ccc2C(c2c(F)cc(N3CCCC4(CCCNCC4)CC3)cc2F)N1CC(C)(F)F] | 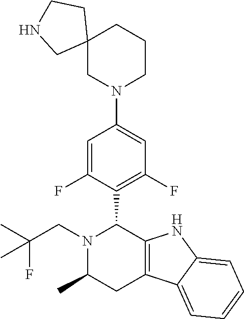 [SMILES: C[C@@H]1Cc2c([nH]c3ccccc23)[C@@H](c2c(F)cc(N3CCCC4(CCNC4)C3)cc2F)N1CC(C)(C)F] |
| 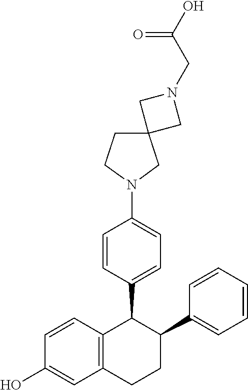 [SMILES: O=C(O)CN1CC2(CCN(c3ccc([C@@H]4c5ccc(O)cc5CC[C@@H]4c4ccccc4)cc3)C2)C1] | 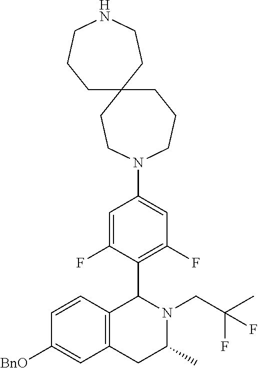 [SMILES: C[C@@H]1Cc2cc(OCc3ccccc3)ccc2C(c2c(F)cc(N3CCCC4(CCCNCC4)CC3)cc2F)N1CC(C)(F)F] | 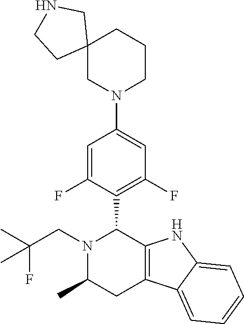 [SMILES: C[C@@H]1Cc2c([nH]c3ccccc23)[C@@H](c2c(F)cc(N3CCCC4(CCNC4)C3)cc2F)N1CC(C)(C)F] |
| 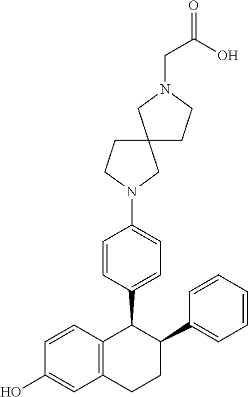 [SMILES: O=C(O)CN1CCC2(CCN(c3ccc([C@@H]4c5ccc(O)cc5CC[C@@H]4c4ccccc4)cc3)C2)C1] | 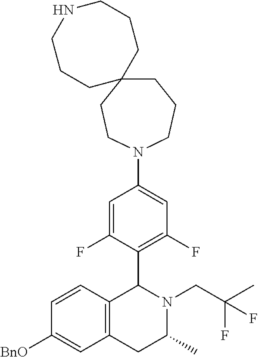 [SMILES: C[C@@H]1Cc2cc(OCc3ccccc3)ccc2C(c2c(F)cc(N3CCCC4(CCCNCCC4)CC3)cc2F)N1CC(C)(F)F] | 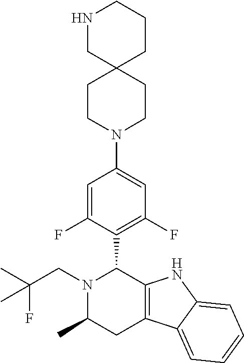 [SMILES: C[C@@H]1Cc2c([nH]c3ccccc23)[C@@H](c2c(F)cc(N3CCC4(CCCNC4)CC3)cc2F)N1CC(C)(C)F] |
| 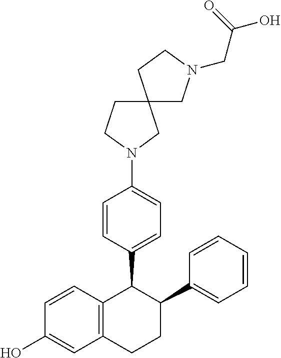 [SMILES: O=C(O)CN1CCC2(CCN(c3ccc([C@@H]4c5ccc(O)cc5CC[C@@H]4c4ccccc4)cc3)C2)C1] | 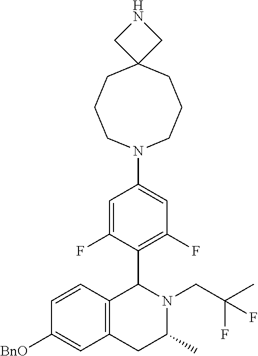 [SMILES: C[C@@H]1Cc2cc(OCc3ccccc3)ccc2C(c2c(F)cc(N3CCCC4(CCC3)CNC4)cc2F)N1CC(C)(F)F] | 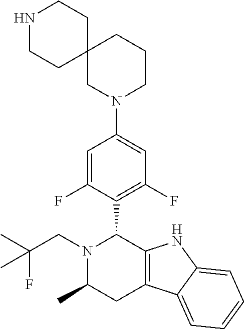 [SMILES: C[C@@H]1Cc2c([nH]c3ccccc23)[C@@H](c2c(F)cc(N3CCCC4(CCNCC4)C3)cc2F)N1CC(C)(C)F] |
| 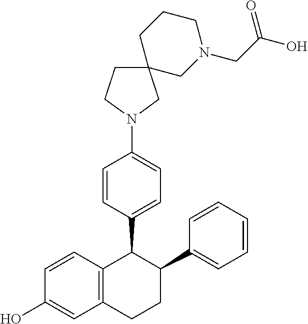 [SMILES: O=C(O)CN1CCCC2(CCN(c3ccc([C@@H]4c5ccc(O)cc5CC[C@@H]4c4ccccc4)cc3)C2)C1] | 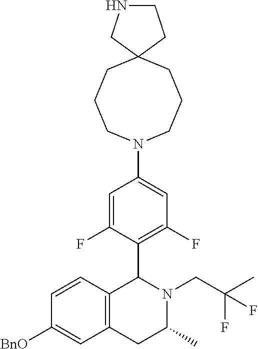 [SMILES: C[C@@H]1Cc2cc(OCc3ccccc3)ccc2C(c2c(F)cc(N3CCCC4(CCC3)CCNC4)cc2F)N1CC(C)(F)F] | 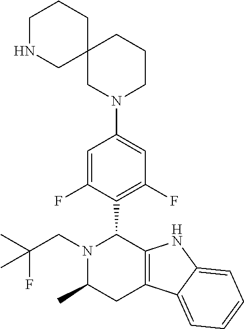 [SMILES: C[C@@H]1Cc2c([nH]c3ccccc23)[C@@H](c2c(F)cc(N3CCCC4(CCCNC4)C3)cc2F)N1CC(C)(C)F] |
| 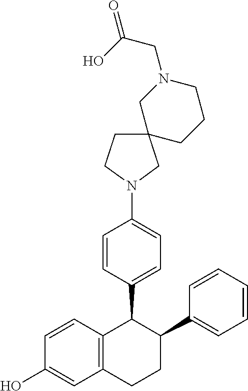 [SMILES: O=C(O)CN1CCCC2(CCN(c3ccc([C@@H]4c5ccc(O)cc5CC[C@@H]4c4ccccc4)cc3)C2)C1] | 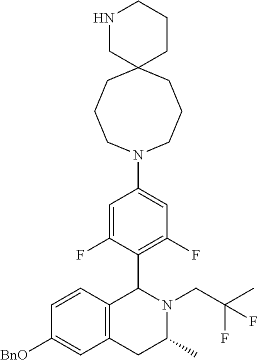 [SMILES: C[C@@H]1Cc2cc(OCc3ccccc3)ccc2C(c2c(F)cc(N3CCCC4(CCCNC4)CCC3)cc2F)N1CC(C)(F)F] | 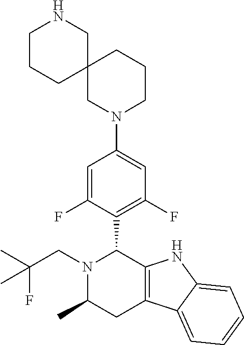 [SMILES: C[C@@H]1Cc2c([nH]c3ccccc23)[C@@H](c2c(F)cc(N3CCCC4(CCCNC4)C3)cc2F)N1CC(C)(C)F] |
| 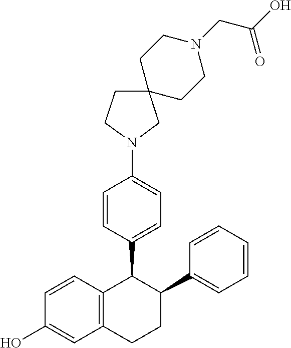 [SMILES: O=C(O)CN1CCC2(CC1)CCN(c1ccc([C@@H]3c4ccc(O)cc4CC[C@@H]3c3ccccc3)cc1)C2] | 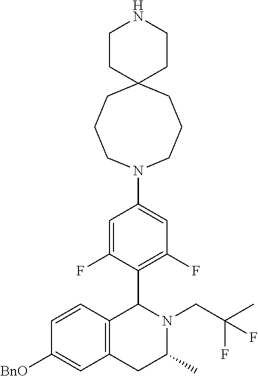 [SMILES: C[C@@H]1Cc2cc(OCc3ccccc3)ccc2C(c2c(F)cc(N3CCCC4(CCC3)CCNCC4)cc2F)N1CC(C)(F)F] | 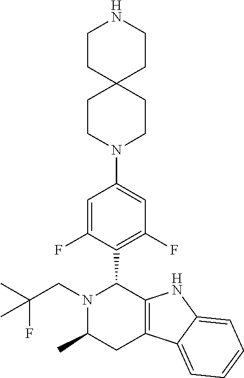 [SMILES: C[C@@H]1Cc2c([nH]c3ccccc23)[C@@H](c2c(F)cc(N3CCC4(CCNCC4)CC3)cc2F)N1CC(C)(C)F] |
| 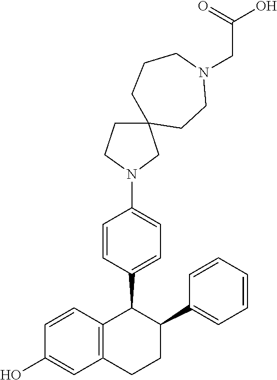 [SMILES: O=C(O)CN1CCCC2(CC1)CCN(c1ccc([C@@H]3c4ccc(O)cc4CC[C@@H]3c3ccccc3)cc1)C2] | 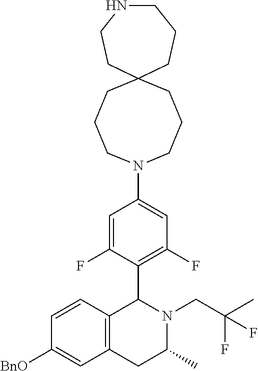 [SMILES: C[C@@H]1Cc2cc(OCc3ccccc3)ccc2C(c2c(F)cc(N3CCCC4(CCCNCC4)CCC3)cc2F)N1CC(C)(F)F] | 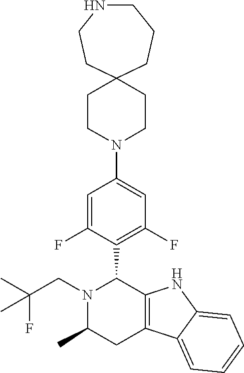 [SMILES: C[C@@H]1Cc2c([nH]c3ccccc23)[C@@H](c2c(F)cc(N3CCC4(CCCNCC4)CC3)cc2F)N1CC(C)(C)F] |
| 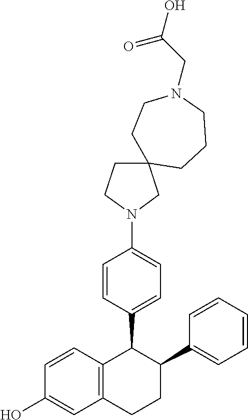 [SMILES: O=C(O)CN1CCCC2(CC1)CCN(c1ccc([C@@H]3c4ccc(O)cc4CC[C@@H]3c3ccccc3)cc1)C2] | 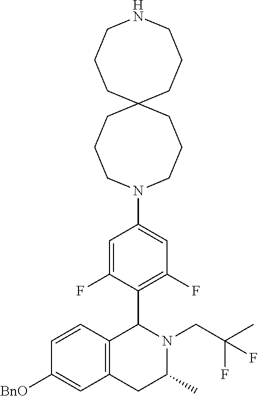 [SMILES: C[C@@H]1Cc2cc(OCc3ccccc3)ccc2C(c2c(F)cc(N3CCCC4(CCCNCCC4)CCC3)cc2F)N1CC(C)(F)F] | 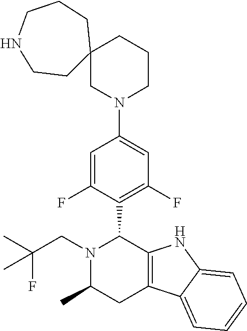 [SMILES: C[C@@H]1Cc2c([nH]c3ccccc23)[C@@H](c2c(F)cc(N3CCCC4(CCCNCC4)C3)cc2F)N1CC(C)(C)F] |
| 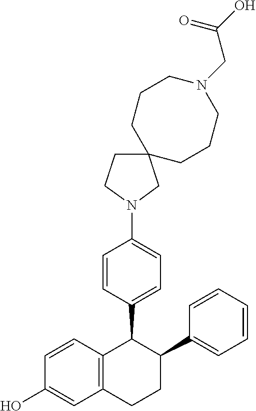 [SMILES: O=C(O)CN1CCCC2(CCC1)CCN(c1ccc([C@@H]3c4ccc(O)cc4CC[C@@H]3c3ccccc3)cc1)C2] | 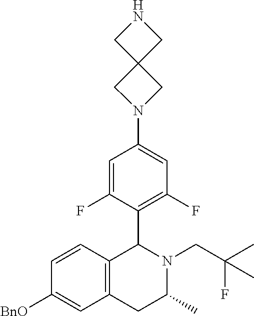 [SMILES: C[C@@H]1Cc2cc(OCc3ccccc3)ccc2C(c2c(F)cc(N3CC4(CNC4)C3)cc2F)N1CC(C)(C)F] | 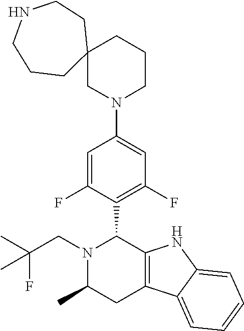 [SMILES: C[C@@H]1Cc2c([nH]c3ccccc23)[C@@H](c2c(F)cc(N3CCCC4(CCCNCC4)C3)cc2F)N1CC(C)(C)F] |
| 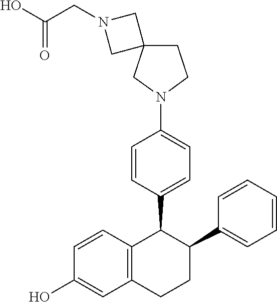 [SMILES: O=C(O)CN1CC2(CCN(c3ccc([C@@H]4c5ccc(O)cc5CC[C@@H]4c4ccccc4)cc3)C2)C1] | 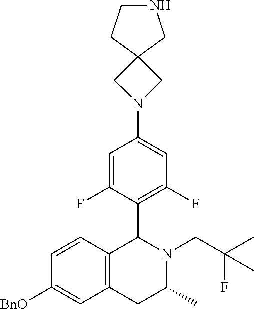 [SMILES: C[C@@H]1Cc2cc(OCc3ccccc3)ccc2C(c2c(F)cc(N3CC4(CCNC4)C3)cc2F)N1CC(C)(C)F] | 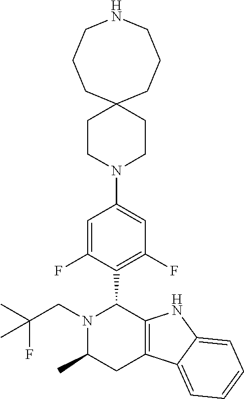 [SMILES: C[C@@H]1Cc2c([nH]c3ccccc23)[C@@H](c2c(F)cc(N3CCC4(CCCNCCC4)CC3)cc2F)N1CC(C)(C)F] |
| 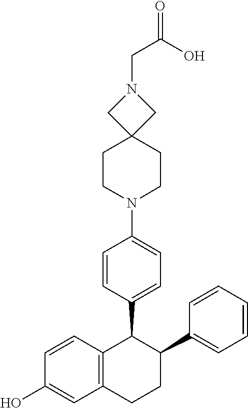 [SMILES: O=C(O)CN1CC2(CCN(c3ccc([C@@H]4c5ccc(O)cc5CC[C@@H]4c4ccccc4)cc3)CC2)C1] | 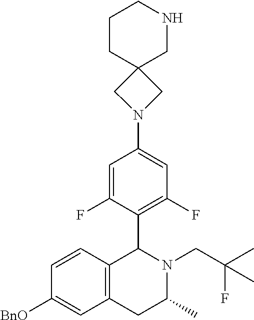 [SMILES: C[C@@H]1Cc2cc(OCc3ccccc3)ccc2C(c2c(F)cc(N3CC4(CCCNC4)C3)cc2F)N1CC(C)(C)F] | 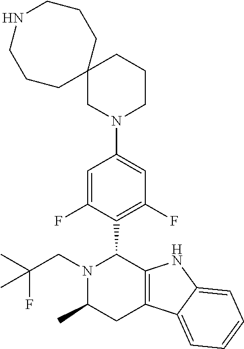 [SMILES: C[C@@H]1Cc2c([nH]c3ccccc23)[C@@H](c2c(F)cc(N3CCCC4(CCCNCCC4)C3)cc2F)N1CC(C)(C)F] |
| 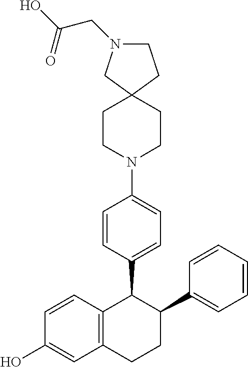 [SMILES: O=C(O)CN1CCC2(CCN(c3ccc([C@@H]4c5ccc(O)cc5CC[C@@H]4c4ccccc4)cc3)CC2)C1] | 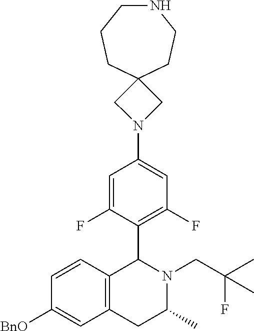 [SMILES: C[C@@H]1Cc2cc(OCc3ccccc3)ccc2C(c2c(F)cc(N3CC4(CCCNCC4)C3)cc2F)N1CC(C)(C)F] | 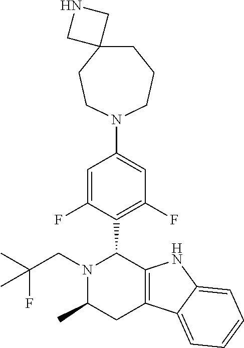 [SMILES: C[C@@H]1Cc2c([nH]c3ccccc23)[C@@H](c2c(F)cc(N3CCCC4(CC3)CNC4)cc2F)N1CC(C)(C)F] |
| 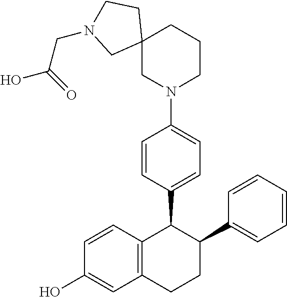 [SMILES: O=C(O)CN1CCC2(CCCN(c3ccc([C@@H]4c5ccc(O)cc5CC[C@@H]4c4ccccc4)cc3)C2)C1] | 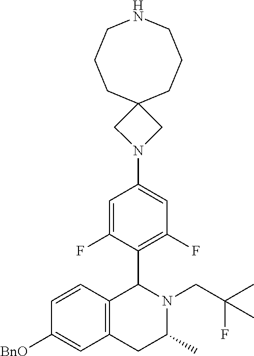 [SMILES: C[C@@H]1Cc2cc(OCc3ccccc3)ccc2C(c2c(F)cc(N3CC4(CCCNCCC4)C3)cc2F)N1CC(C)(C)F] | 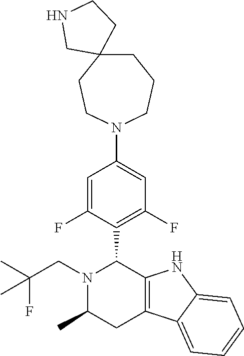 [SMILES: C[C@@H]1Cc2c([nH]c3ccccc23)[C@@H](c2c(F)cc(N3CCCC4(CCNC4)CC3)cc2F)N1CC(C)(C)F] |
| 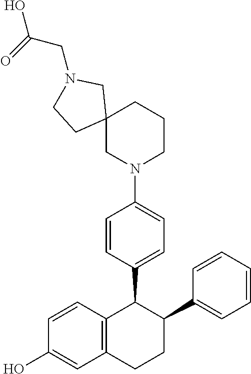 [SMILES: O=C(O)CN1CCC2(CCCN(c3ccc([C@@H]4c5ccc(O)cc5CC[C@@H]4c4ccccc4)cc3)C2)C1] | 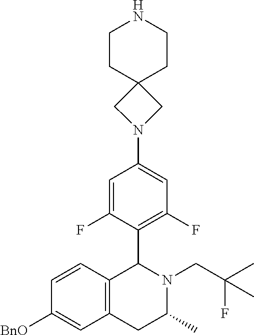 [SMILES: C[C@@H]1Cc2cc(OCc3ccccc3)ccc2C(c2c(F)cc(N3CC4(CCNCC4)C3)cc2F)N1CC(C)(C)F] | 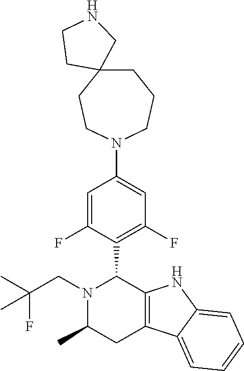 [SMILES: C[C@@H]1Cc2c([nH]c3ccccc23)[C@@H](c2c(F)cc(N3CCCC4(CCNC4)CC3)cc2F)N1CC(C)(C)F] |
| 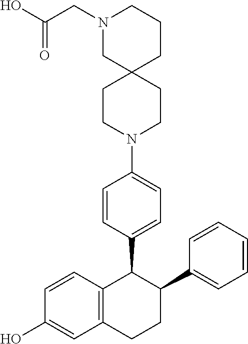 [SMILES: O=C(O)CN1CCCC2(CCN(c3ccc([C@@H]4c5ccc(O)cc5CC[C@@H]4c4ccccc4)cc3)CC2)C1] | 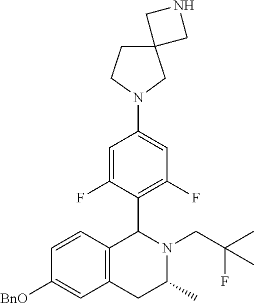 [SMILES: C[C@@H]1Cc2cc(OCc3ccccc3)ccc2C(c2c(F)cc(N3CCC4(CNC4)C3)cc2F)N1CC(C)(C)F] | 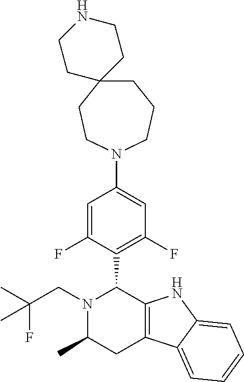 [SMILES: C[C@@H]1Cc2c([nH]c3ccccc23)[C@@H](c2c(F)cc(N3CCCC4(CCNCC4)CC3)cc2F)N1CC(C)(C)F] |
| 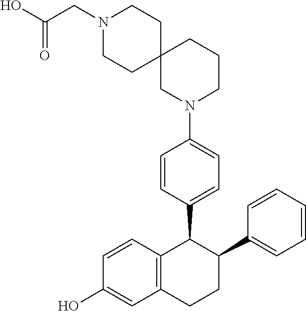 [SMILES: O=C(O)CN1CCC2(CCCN(c3ccc([C@@H]4c5ccc(O)cc5CC[C@@H]4c4ccccc4)cc3)C2)CC1] | 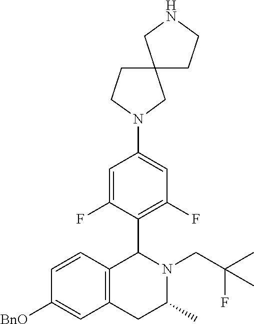 [SMILES: C[C@@H]1Cc2cc(OCc3ccccc3)ccc2C(c2c(F)cc(N3CCC4(CCNC4)C3)cc2F)N1CC(C)(C)F] | 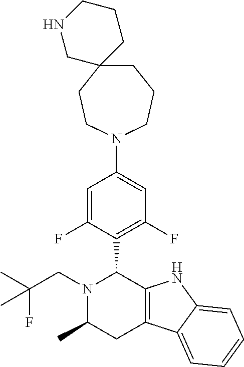 [SMILES: C[C@@H]1Cc2c([nH]c3ccccc23)[C@@H](c2c(F)cc(N3CCCC4(CCCNC4)CC3)cc2F)N1CC(C)(C)F] |
| 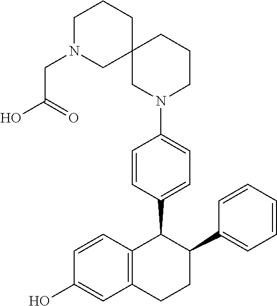 [SMILES: O=C(O)CN1CCCC2(CCCN(c3ccc([C@@H]4c5ccc(O)cc5CC[C@@H]4c4ccccc4)cc3)C2)C1] | 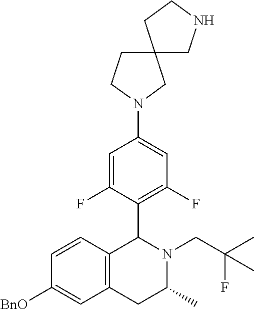 [SMILES: C[C@@H]1Cc2cc(OCc3ccccc3)ccc2C(c2c(F)cc(N3CCC4(CCNC4)C3)cc2F)N1CC(C)(C)F] | 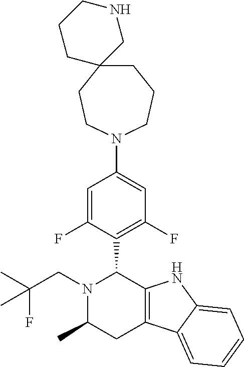 [SMILES: C[C@@H]1Cc2c([nH]c3ccccc23)[C@@H](c2c(F)cc(N3CCCC4(CCCNC4)CC3)cc2F)N1CC(C)(C)F] |
| 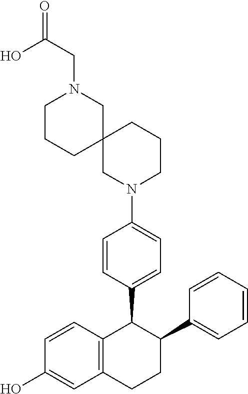 [SMILES: O=C(O)CN1CCCC2(CCCN(c3ccc([C@@H]4c5ccc(O)cc5CC[C@@H]4c4ccccc4)cc3)C2)C1] | 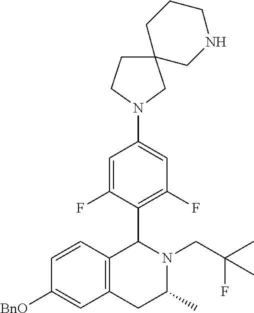 [SMILES: C[C@@H]1Cc2cc(OCc3ccccc3)ccc2C(c2c(F)cc(N3CCC4(CCCNC4)C3)cc2F)N1CC(C)(C)F] | 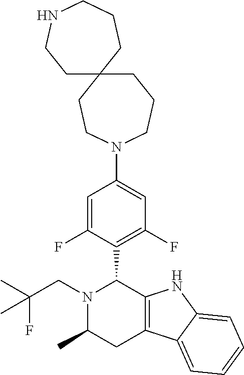 [SMILES: C[C@@H]1Cc2c([nH]c3ccccc23)[C@@H](c2c(F)cc(N3CCCC4(CCCNCC4)CC3)cc2F)N1CC(C)(C)F] |
| 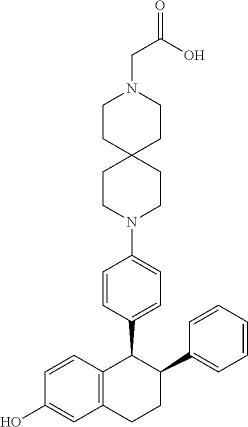 [SMILES: O=C(O)CN1CCC2(CC1)CCN(c1ccc([C@@H]3c4ccc(O)cc4CC[C@@H]3c3ccccc3)cc1)CC2] | 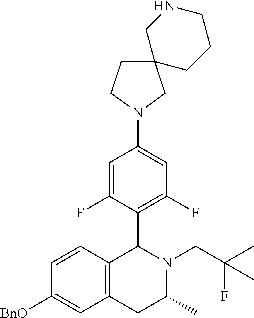 [SMILES: C[C@@H]1Cc2cc(OCc3ccccc3)ccc2C(c2c(F)cc(N3CCC4(CCCNC4)C3)cc2F)N1CC(C)(C)F] | 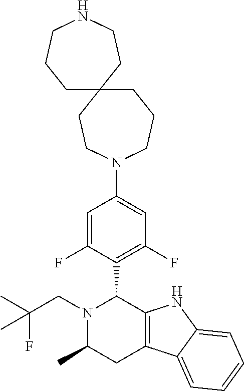 [SMILES: C[C@@H]1Cc2c([nH]c3ccccc23)[C@@H](c2c(F)cc(N3CCCC4(CCCNCC4)CC3)cc2F)N1CC(C)(C)F] |
| 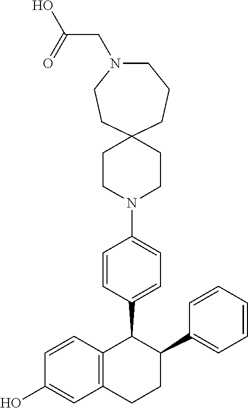 [SMILES: O=C(O)CN1CCCC2(CC1)CCN(c1ccc([C@@H]3c4ccc(O)cc4CC[C@@H]3c3ccccc3)cc1)CC2] | 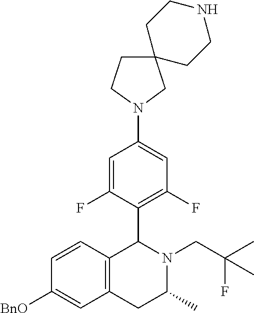 [SMILES: C[C@@H]1Cc2cc(OCc3ccccc3)ccc2C(c2c(F)cc(N3CCC4(CCNCC4)C3)cc2F)N1CC(C)(C)F] | 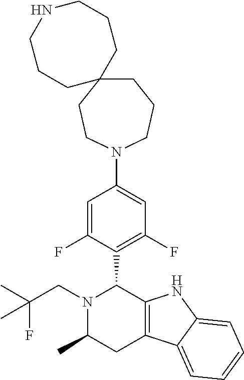 [SMILES: C[C@@H]1Cc2c([nH]c3ccccc23)[C@@H](c2c(F)cc(N3CCCC4(CCCNCCC4)CC3)cc2F)N1CC(C)(C)F] |
| 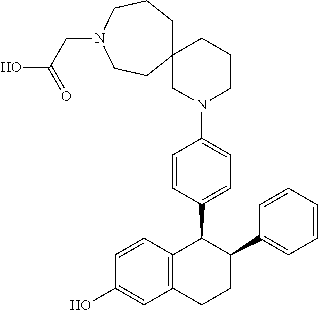 [SMILES: O=C(O)CN1CCCC2(CCCN(c3ccc([C@@H]4c5ccc(O)cc5CC[C@@H]4c4ccccc4)cc3)C2)CC1] | 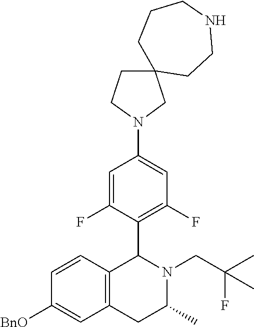 [SMILES: C[C@@H]1Cc2cc(OCc3ccccc3)ccc2C(c2c(F)cc(N3CCC4(CCCNCC4)C3)cc2F)N1CC(C)(C)F] | 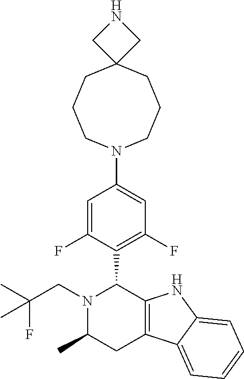 [SMILES: C[C@@H]1Cc2c([nH]c3ccccc23)[C@@H](c2c(F)cc(N3CCCC4(CCC3)CNC4)cc2F)N1CC(C)(C)F] |
| 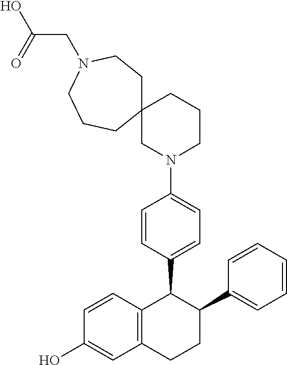 [SMILES: O=C(O)CN1CCCC2(CCCN(c3ccc([C@@H]4c5ccc(O)cc5CC[C@@H]4c4ccccc4)cc3)C2)CC1] | 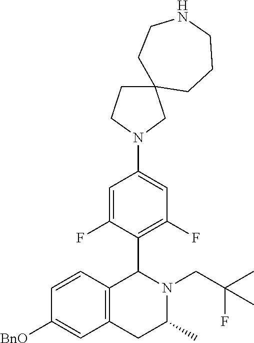 [SMILES: C[C@@H]1Cc2cc(OCc3ccccc3)ccc2C(c2c(F)cc(N3CCC4(CCCNCC4)C3)cc2F)N1CC(C)(C)F] | 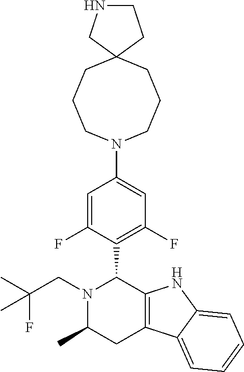 [SMILES: C[C@@H]1Cc2c([nH]c3ccccc23)[C@@H](c2c(F)cc(N3CCCC4(CCC3)CCNC4)cc2F)N1CC(C)(C)F] |
| 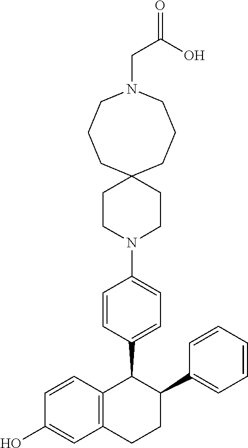 [SMILES: O=C(O)CN1CCCC2(CCC1)CCN(c1ccc([C@@H]3c4ccc(O)cc4CC[C@@H]3c3ccccc3)cc1)CC2] | 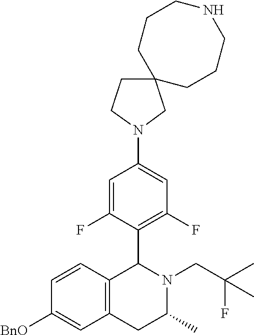 [SMILES: C[C@@H]1Cc2cc(OCc3ccccc3)ccc2C(c2c(F)cc(N3CCC4(CCCNCCC4)C3)cc2F)N1CC(C)(C)F] | 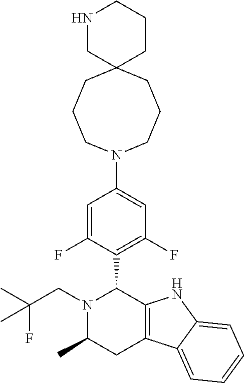 [SMILES: C[C@@H]1Cc2c([nH]c3ccccc23)[C@@H](c2c(F)cc(N3CCCC4(CCCNC4)CCC3)cc2F)N1CC(C)(C)F] |
| 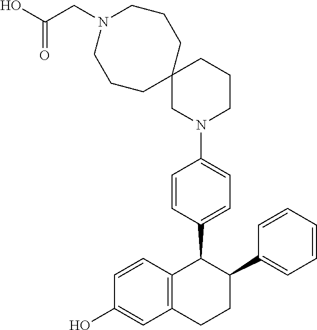 [SMILES: O=C(O)CN1CCCC2(CCC1)CCCN(c1ccc([C@@H]3c4ccc(O)cc4CC[C@@H]3c3ccccc3)cc1)C2] | 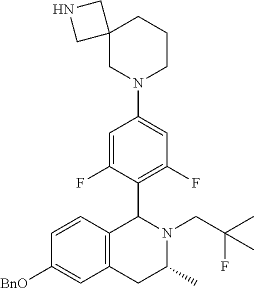 [SMILES: C[C@@H]1Cc2cc(OCc3ccccc3)ccc2C(c2c(F)cc(N3CCCC4(CNC4)C3)cc2F)N1CC(C)(C)F] | 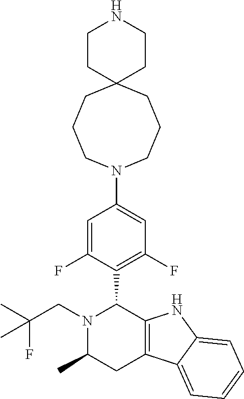 [SMILES: C[C@@H]1Cc2c([nH]c3ccccc23)[C@@H](c2c(F)cc(N3CCCC4(CCC3)CCNCC4)cc2F)N1CC(C)(C)F] |
| 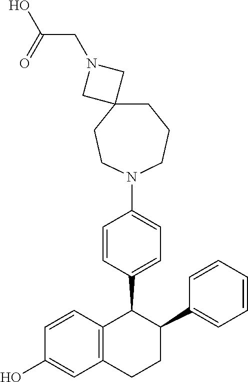 [SMILES: O=C(O)CN1CC2(CCCN(c3ccc([C@@H]4c5ccc(O)cc5CC[C@@H]4c4ccccc4)cc3)CC2)C1] | 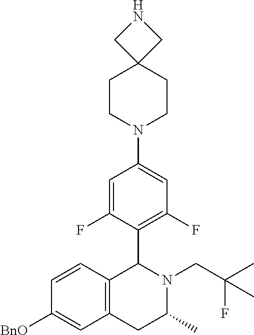 [SMILES: C[C@@H]1Cc2cc(OCc3ccccc3)ccc2C(c2c(F)cc(N3CCC4(CC3)CNC4)cc2F)N1CC(C)(C)F] | 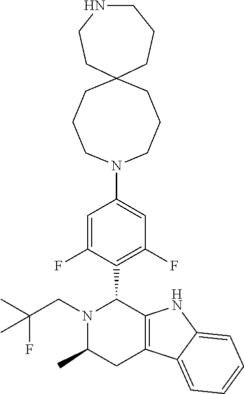 [SMILES: C[C@@H]1Cc2c([nH]c3ccccc23)[C@@H](c2c(F)cc(N3CCCC4(CCCNCC4)CCC3)cc2F)N1CC(C)(C)F] |
| 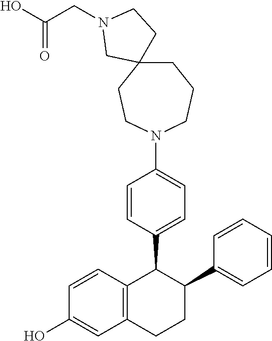 [SMILES: O=C(O)CN1CCC2(CCCN(c3ccc([C@@H]4c5ccc(O)cc5CC[C@@H]4c4ccccc4)cc3)CC2)C1] | 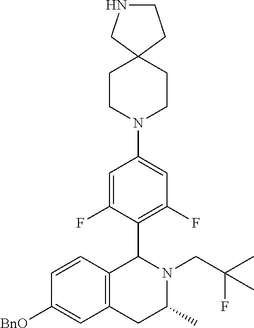 [SMILES: C[C@@H]1Cc2cc(OCc3ccccc3)ccc2C(c2c(F)cc(N3CCC4(CCNC4)CC3)cc2F)N1CC(C)(C)F] | 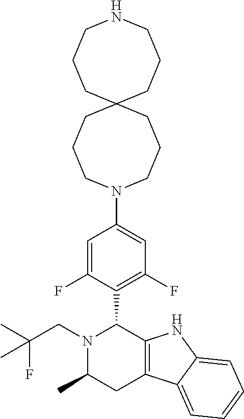 [SMILES: C[C@@H]1Cc2c([nH]c3ccccc23)[C@@H](c2c(F)cc(N3CCCC4(CCCNCCC4)CCC3)cc2F)N1CC(C)(C)F] |
| 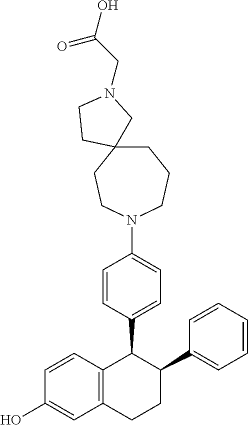 [SMILES: O=C(O)CN1CCC2(CCCN(c3ccc([C@@H]4c5ccc(O)cc5CC[C@@H]4c4ccccc4)cc3)CC2)C1] | 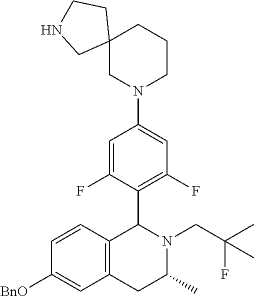 [SMILES: C[C@@H]1Cc2cc(OCc3ccccc3)ccc2C(c2c(F)cc(N3CCCC4(CCNC4)C3)cc2F)N1CC(C)(C)F] | 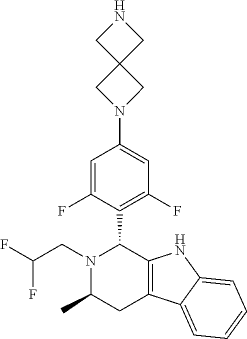 [SMILES: C[C@@H]1Cc2c([nH]c3ccccc23)[C@@H](c2c(F)cc(N3CC4(CNC4)C3)cc2F)N1CC(F)F] |
| 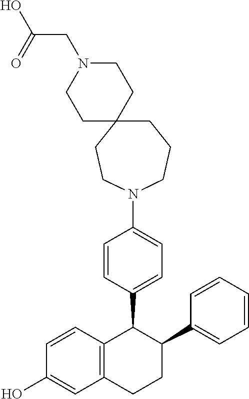 [SMILES: O=C(O)CN1CCC2(CCCN(c3ccc([C@@H]4c5ccc(O)cc5CC[C@@H]4c4ccccc4)cc3)CC2)CC1] | 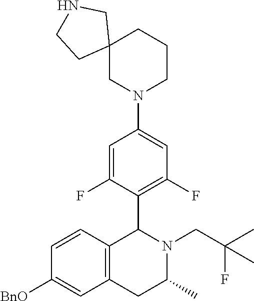 [SMILES: C[C@@H]1Cc2cc(OCc3ccccc3)ccc2C(c2c(F)cc(N3CCCC4(CCNC4)C3)cc2F)N1CC(C)(C)F] | 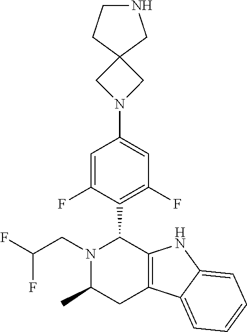 [SMILES: C[C@@H]1Cc2c([nH]c3ccccc23)[C@@H](c2c(F)cc(N3CC4(CCNC4)C3)cc2F)N1CC(F)F] |
| 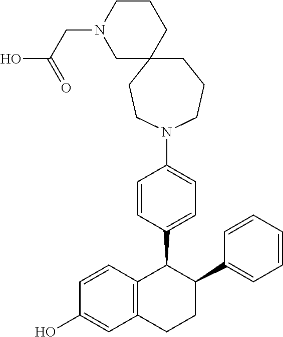 [SMILES: O=C(O)CN1CCCC2(CCCN(c3ccc([C@@H]4c5ccc(O)cc5CC[C@@H]4c4ccccc4)cc3)CC2)C1] | 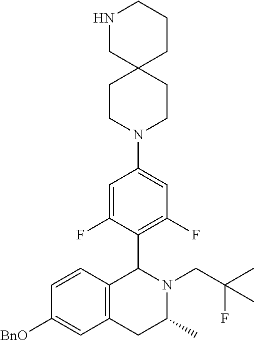 [SMILES: C[C@@H]1Cc2cc(OCc3ccccc3)ccc2C(c2c(F)cc(N3CCC4(CCCNC4)CC3)cc2F)N1CC(C)(C)F] | 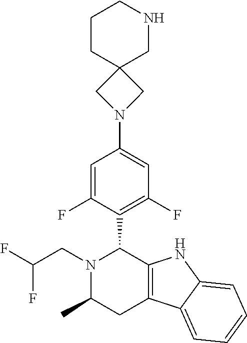 [SMILES: C[C@@H]1Cc2c([nH]c3ccccc23)[C@@H](c2c(F)cc(N3CC4(CCCNC4)C3)cc2F)N1CC(F)F] |
| 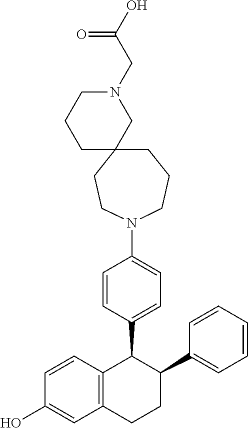 [SMILES: O=C(O)CN1CCCC2(CCCN(c3ccc([C@@H]4c5ccc(O)cc5CC[C@@H]4c4ccccc4)cc3)CC2)C1] | 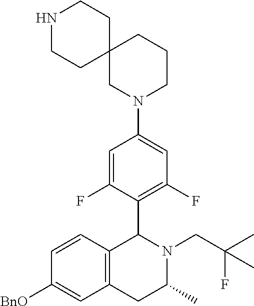 [SMILES: C[C@@H]1Cc2cc(OCc3ccccc3)ccc2C(c2c(F)cc(N3CCCC4(CCNCC4)C3)cc2F)N1CC(C)(C)F] | 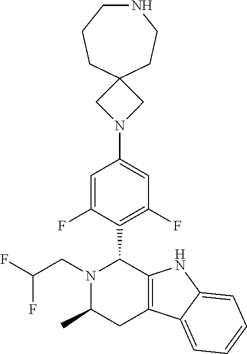 [SMILES: C[C@@H]1Cc2c([nH]c3ccccc23)[C@@H](c2c(F)cc(N3CC4(CCCNCC4)C3)cc2F)N1CC(F)F] |
| 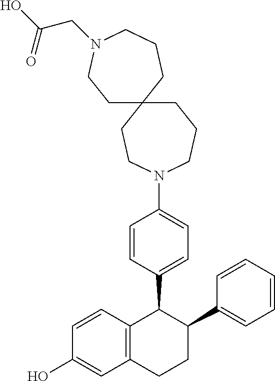 [SMILES: O=C(O)CN1CCCC2(CCCN(c3ccc([C@@H]4c5ccc(O)cc5CC[C@@H]4c4ccccc4)cc3)CC2)CC1] | 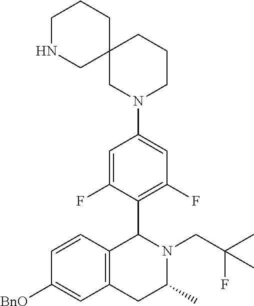 [SMILES: C[C@@H]1Cc2cc(OCc3ccccc3)ccc2C(c2c(F)cc(N3CCCC4(CCCNC4)C3)cc2F)N1CC(C)(C)F] | 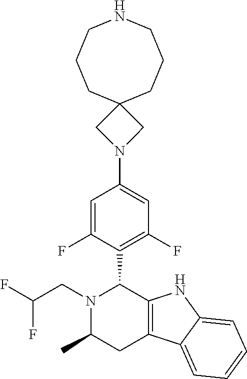 [SMILES: C[C@@H]1Cc2c([nH]c3ccccc23)[C@@H](c2c(F)cc(N3CC4(CCCNCCC4)C3)cc2F)N1CC(F)F] |
| 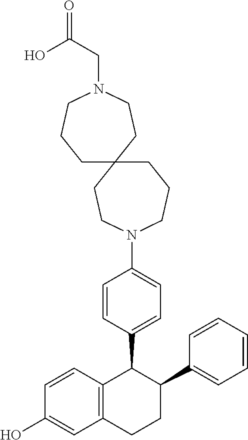 [SMILES: O=C(O)CN1CCCC2(CCCN(c3ccc([C@@H]4c5ccc(O)cc5CC[C@@H]4c4ccccc4)cc3)CC2)CC1] | 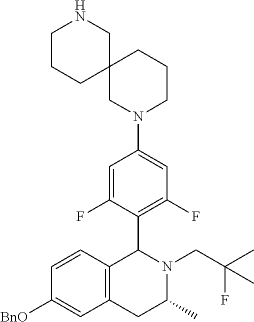 [SMILES: C[C@@H]1Cc2cc(OCc3ccccc3)ccc2C(c2c(F)cc(N3CCCC4(CCCNC4)C3)cc2F)N1CC(C)(C)F] | 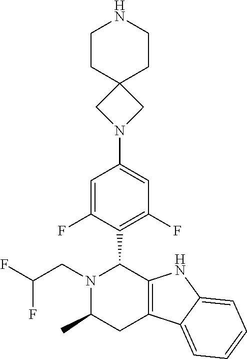 [SMILES: C[C@@H]1Cc2c([nH]c3ccccc23)[C@@H](c2c(F)cc(N3CC4(CCNCC4)C3)cc2F)N1CC(F)F] |
| 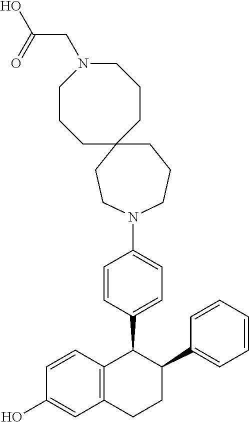 [SMILES: O=C(O)CN1CCCC2(CCC1)CCCN(c1ccc([C@@H]3c4ccc(O)cc4CC[C@@H]3c3ccccc3)cc1)CC2] | 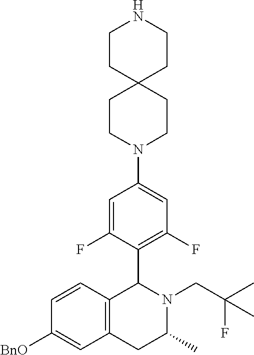 [SMILES: C[C@@H]1Cc2cc(OCc3ccccc3)ccc2C(c2c(F)cc(N3CCC4(CCNCC4)CC3)cc2F)N1CC(C)(C)F] | 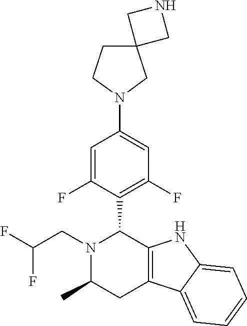 [SMILES: C[C@@H]1Cc2c([nH]c3ccccc23)[C@@H](c2c(F)cc(N3CCC4(CNC4)C3)cc2F)N1CC(F)F] |
| 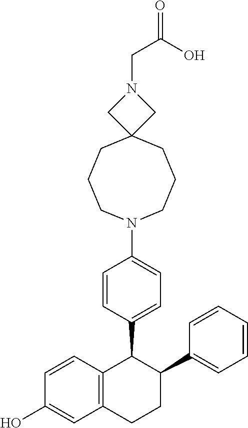 [SMILES: O=C(O)CN1CC2(CCCN(c3ccc([C@@H]4c5ccc(O)cc5CC[C@@H]4c4ccccc4)cc3)CCC2)C1] | 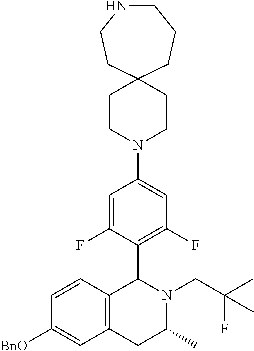 [SMILES: C[C@@H]1Cc2cc(OCc3ccccc3)ccc2C(c2c(F)cc(N3CCC4(CCCNCC4)CC3)cc2F)N1CC(C)(C)F] | 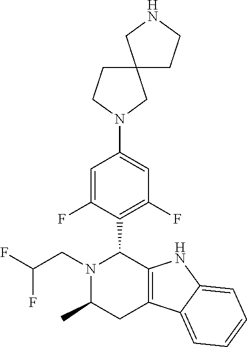 [SMILES: C[C@@H]1Cc2c([nH]c3ccccc23)[C@@H](c2c(F)cc(N3CCC4(CCNC4)C3)cc2F)N1CC(F)F] |
| 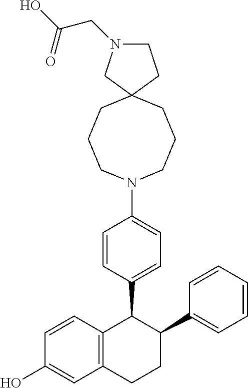 [SMILES: O=C(O)CN1CCC2(CCCN(c3ccc([C@@H]4c5ccc(O)cc5CC[C@@H]4c4ccccc4)cc3)CCC2)C1] | 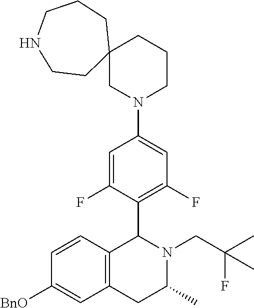 [SMILES: C[C@@H]1Cc2cc(OCc3ccccc3)ccc2C(c2c(F)cc(N3CCCC4(CCCNCC4)C3)cc2F)N1CC(C)(C)F] | 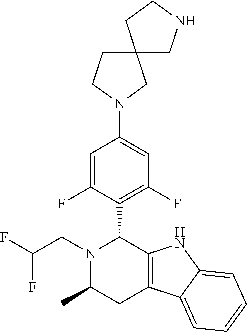 [SMILES: C[C@@H]1Cc2c([nH]c3ccccc23)[C@@H](c2c(F)cc(N3CCC4(CCNC4)C3)cc2F)N1CC(F)F] |
| 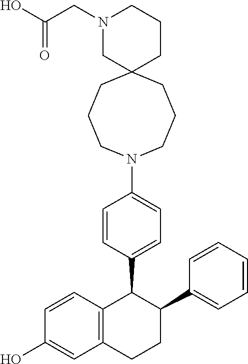 [SMILES: O=C(O)CN1CCCC2(CCCN(c3ccc([C@@H]4c5ccc(O)cc5CC[C@@H]4c4ccccc4)cc3)CCC2)C1] | 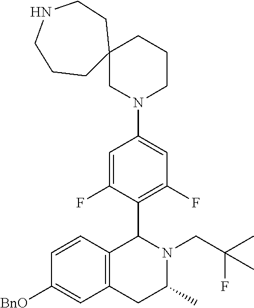 [SMILES: C[C@@H]1Cc2cc(OCc3ccccc3)ccc2C(c2c(F)cc(N3CCCC4(CCCNCC4)C3)cc2F)N1CC(C)(C)F] | 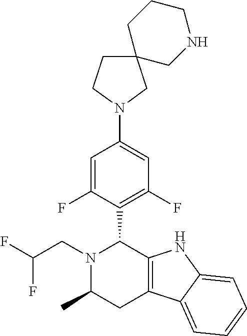 [SMILES: C[C@@H]1Cc2c([nH]c3ccccc23)[C@@H](c2c(F)cc(N3CCC4(CCCNC4)C3)cc2F)N1CC(F)F] |
| 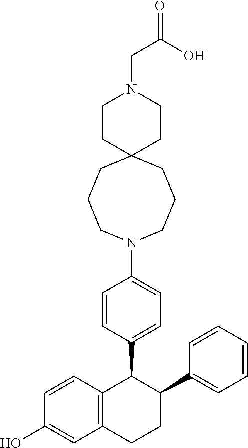 [SMILES: O=C(O)CN1CCC2(CCCN(c3ccc([C@@H]4c5ccc(O)cc5CC[C@@H]4c4ccccc4)cc3)CCC2)CC1] | 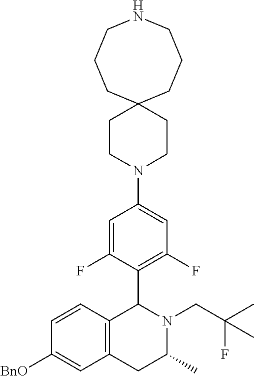 [SMILES: C[C@@H]1Cc2cc(OCc3ccccc3)ccc2C(c2c(F)cc(N3CCC4(CCCNCCC4)CC3)cc2F)N1CC(C)(C)F] | 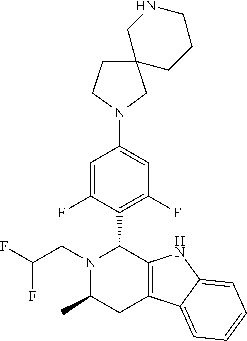 [SMILES: C[C@@H]1Cc2c([nH]c3ccccc23)[C@@H](c2c(F)cc(N3CCC4(CCCNC4)C3)cc2F)N1CC(F)F] |
| 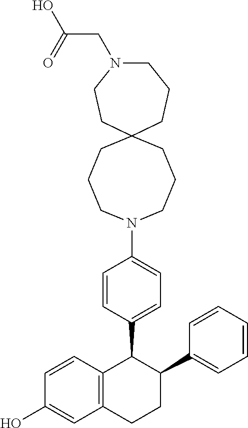 [SMILES: O=C(O)CN1CCCC2(CCCN(c3ccc([C@@H]4c5ccc(O)cc5CC[C@@H]4c4ccccc4)cc3)CCC2)CC1] | 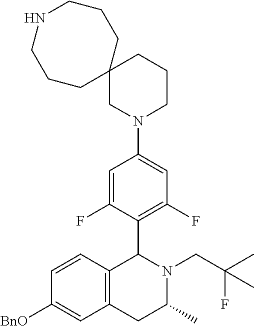 [SMILES: C[C@@H]1Cc2cc(OCc3ccccc3)ccc2C(c2c(F)cc(N3CCCC4(CCCNCCC4)C3)cc2F)N1CC(C)(C)F] | 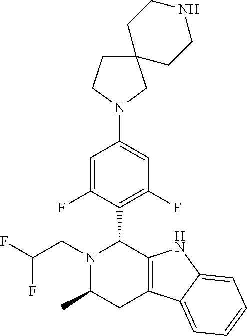 [SMILES: C[C@@H]1Cc2c([nH]c3ccccc23)[C@@H](c2c(F)cc(N3CCC4(CCNCC4)C3)cc2F)N1CC(F)F] |
| 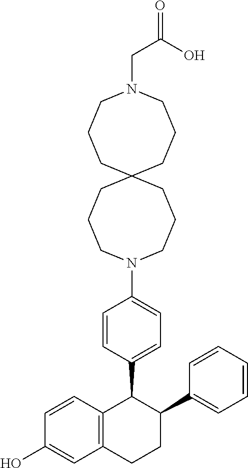 [SMILES: O=C(O)CN1CCCC2(CCC1)CCCN(c1ccc([C@@H]3c4ccc(O)cc4CC[C@@H]3c3ccccc3)cc1)CCC2] | 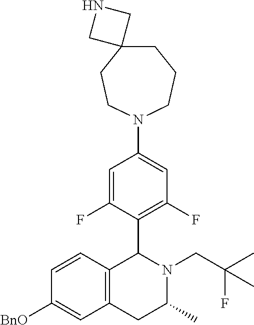 [SMILES: C[C@@H]1Cc2cc(OCc3ccccc3)ccc2C(c2c(F)cc(N3CCCC4(CC3)CNC4)cc2F)N1CC(C)(C)F] | 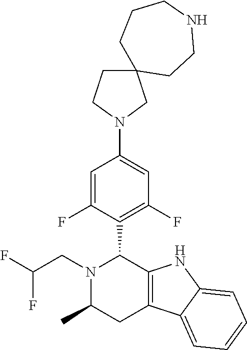 [SMILES: C[C@@H]1Cc2c([nH]c3ccccc23)[C@@H](c2c(F)cc(N3CCC4(CCCNCC4)C3)cc2F)N1CC(F)F] |
| 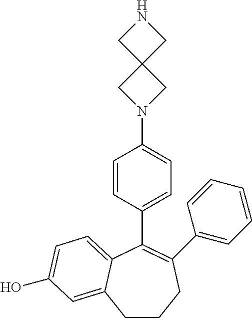 [SMILES: Oc1ccc2c(c1)CCCC(c1ccccc1)=C2c1ccc(N2CC3(CNC3)C2)cc1] | 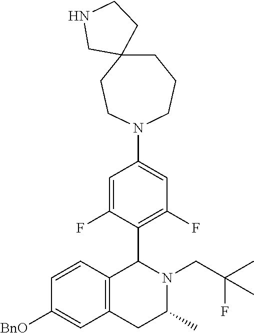 [SMILES: C[C@@H]1Cc2cc(OCc3ccccc3)ccc2C(c2c(F)cc(N3CCCC4(CCNC4)CC3)cc2F)N1CC(C)(C)F] | 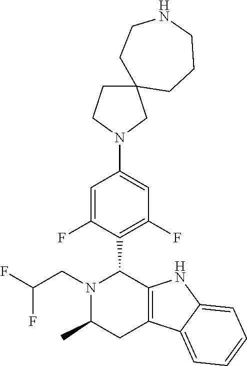 [SMILES: C[C@@H]1Cc2c([nH]c3ccccc23)[C@@H](c2c(F)cc(N3CCC4(CCCNCC4)C3)cc2F)N1CC(F)F] |
| 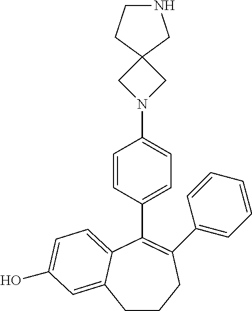 [SMILES: Oc1ccc2c(c1)CCCC(c1ccccc1)=C2c1ccc(N2CC3(CCNC3)C2)cc1] | 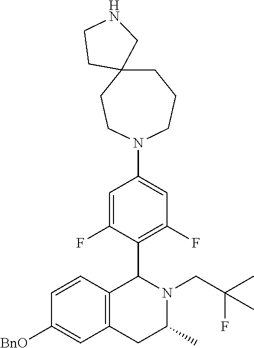 [SMILES: C[C@@H]1Cc2cc(OCc3ccccc3)ccc2C(c2c(F)cc(N3CCCC4(CCNC4)CC3)cc2F)N1CC(C)(C)F] | 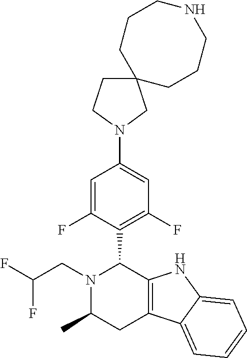 [SMILES: C[C@@H]1Cc2c([nH]c3ccccc23)[C@@H](c2c(F)cc(N3CCC4(CCCNCCC4)C3)cc2F)N1CC(F)F] |
| 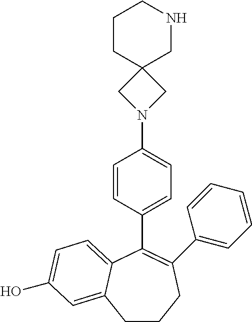 [SMILES: Oc1ccc2c(c1)CCCC(c1ccccc1)=C2c1ccc(N2CC3(CCCNC3)C2)cc1] | 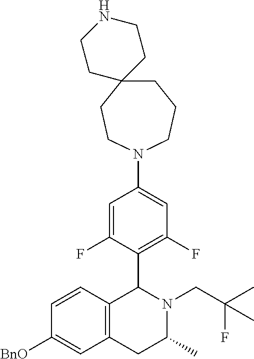 [SMILES: C[C@@H]1Cc2cc(OCc3ccccc3)ccc2C(c2c(F)cc(N3CCCC4(CCNCC4)CC3)cc2F)N1CC(C)(C)F] | 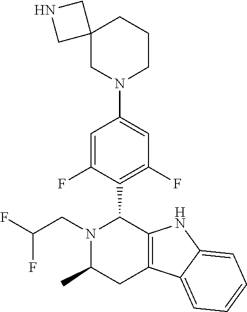 [SMILES: C[C@@H]1Cc2c([nH]c3ccccc23)[C@@H](c2c(F)cc(N3CCCC4(CNC4)C3)cc2F)N1CC(F)F] |
| 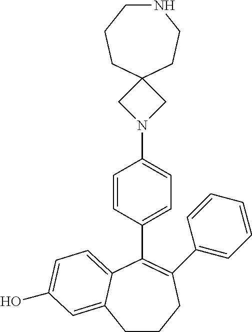 [SMILES: Oc1ccc2c(c1)CCCC(c1ccccc1)=C2c1ccc(N2CC3(CCCNCC3)C2)cc1] | 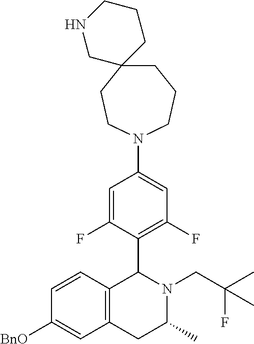 [SMILES: C[C@@H]1Cc2cc(OCc3ccccc3)ccc2C(c2c(F)cc(N3CCCC4(CCCNC4)CC3)cc2F)N1CC(C)(C)F] | 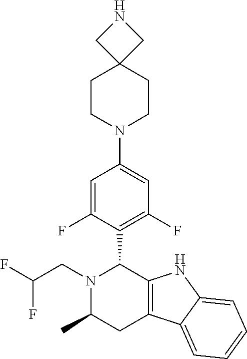 [SMILES: C[C@@H]1Cc2c([nH]c3ccccc23)[C@@H](c2c(F)cc(N3CCC4(CC3)CNC4)cc2F)N1CC(F)F] |
| 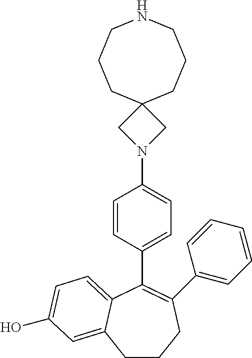 [SMILES: Oc1ccc2c(c1)CCCC(c1ccccc1)=C2c1ccc(N2CC3(CCCNCCC3)C2)cc1] | 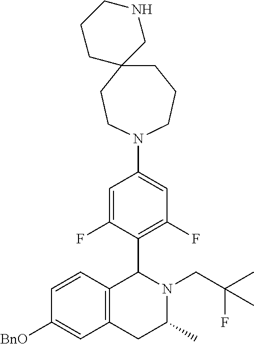 [SMILES: C[C@@H]1Cc2cc(OCc3ccccc3)ccc2C(c2c(F)cc(N3CCCC4(CCCNC4)CC3)cc2F)N1CC(C)(C)F] | 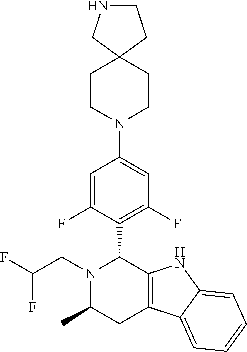 [SMILES: C[C@@H]1Cc2c([nH]c3ccccc23)[C@@H](c2c(F)cc(N3CCC4(CCNC4)CC3)cc2F)N1CC(F)F] |
| 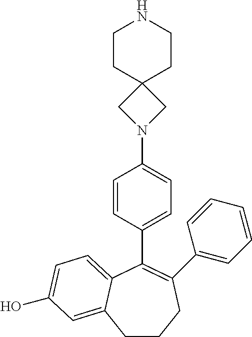 [SMILES: Oc1ccc2c(c1)CCCC(c1ccccc1)=C2c1ccc(N2CC3(CCNCC3)C2)cc1] | 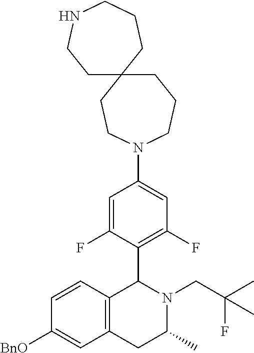 [SMILES: C[C@@H]1Cc2cc(OCc3ccccc3)ccc2C(c2c(F)cc(N3CCCC4(CCCNCC4)CC3)cc2F)N1CC(C)(C)F] | 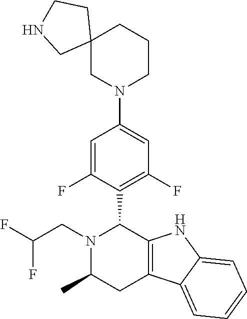 [SMILES: C[C@@H]1Cc2c([nH]c3ccccc23)[C@@H](c2c(F)cc(N3CCCC4(CCNC4)C3)cc2F)N1CC(F)F] |
| 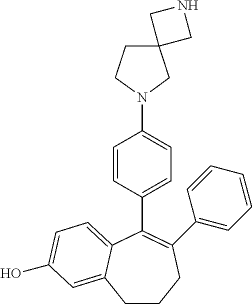 [SMILES: Oc1ccc2c(c1)CCCC(c1ccccc1)=C2c1ccc(N2CCC3(CNC3)C2)cc1] | 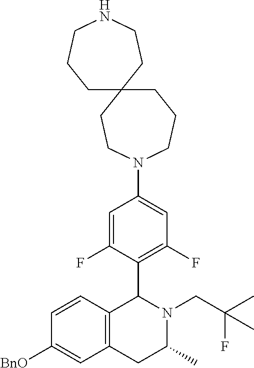 [SMILES: C[C@@H]1Cc2cc(OCc3ccccc3)ccc2C(c2c(F)cc(N3CCCC4(CCCNCC4)CC3)cc2F)N1CC(C)(C)F] | 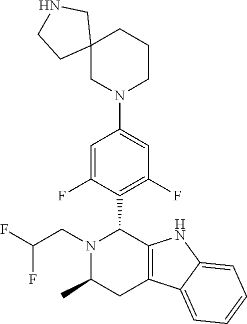 [SMILES: C[C@@H]1Cc2c([nH]c3ccccc23)[C@@H](c2c(F)cc(N3CCCC4(CCNC4)C3)cc2F)N1CC(F)F] |
| 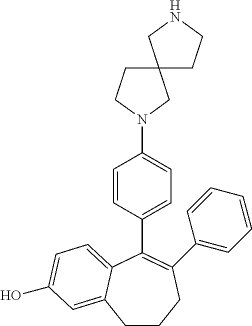 [SMILES: Oc1ccc2c(c1)CCCC(c1ccccc1)=C2c1ccc(N2CCC3(CCNC3)C2)cc1] | 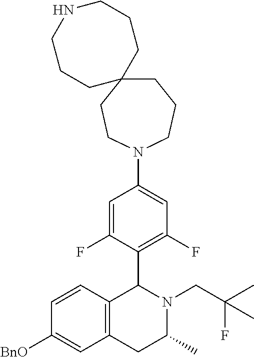 [SMILES: C[C@@H]1Cc2cc(OCc3ccccc3)ccc2C(c2c(F)cc(N3CCCC4(CCCNCCC4)CC3)cc2F)N1CC(C)(C)F] | 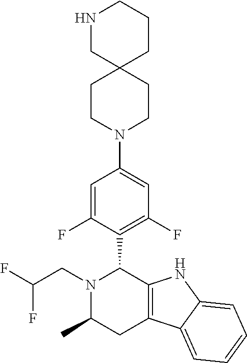 [SMILES: C[C@@H]1Cc2c([nH]c3ccccc23)[C@@H](c2c(F)cc(N3CCC4(CCCNC4)CC3)cc2F)N1CC(F)F] |
| 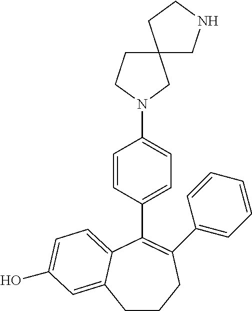 [SMILES: Oc1ccc2c(c1)CCCC(c1ccccc1)=C2c1ccc(N2CCC3(CCNC3)C2)cc1] | 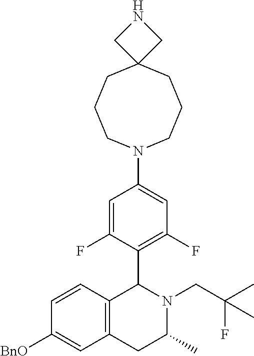 [SMILES: C[C@@H]1Cc2cc(OCc3ccccc3)ccc2C(c2c(F)cc(N3CCCC4(CCC3)CNC4)cc2F)N1CC(C)(C)F] | 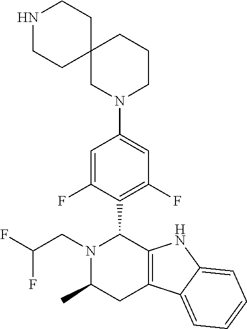 [SMILES: C[C@@H]1Cc2c([nH]c3ccccc23)[C@@H](c2c(F)cc(N3CCCC4(CCNCC4)C3)cc2F)N1CC(F)F] |
| 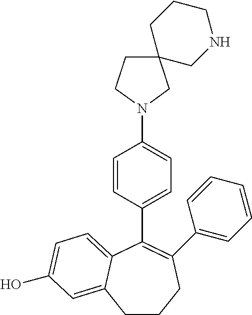 [SMILES: Oc1ccc2c(c1)CCCC(c1ccccc1)=C2c1ccc(N2CCC3(CCCNC3)C2)cc1] | 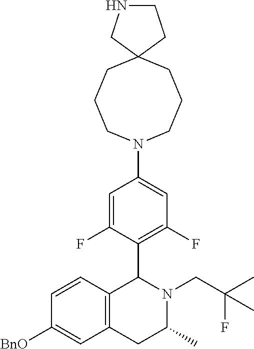 [SMILES: C[C@@H]1Cc2cc(OCc3ccccc3)ccc2C(c2c(F)cc(N3CCCC4(CCC3)CCNC4)cc2F)N1CC(C)(C)F] | 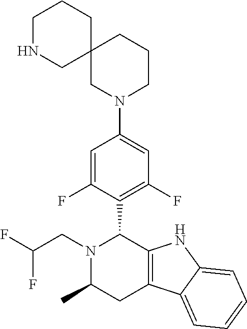 [SMILES: C[C@@H]1Cc2c([nH]c3ccccc23)[C@@H](c2c(F)cc(N3CCCC4(CCCNC4)C3)cc2F)N1CC(F)F] |
| 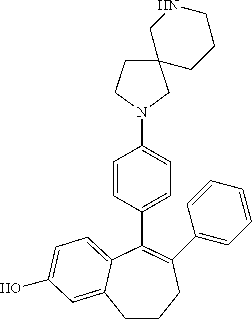 [SMILES: Oc1ccc2c(c1)CCCC(c1ccccc1)=C2c1ccc(N2CCC3(CCCNC3)C2)cc1] | 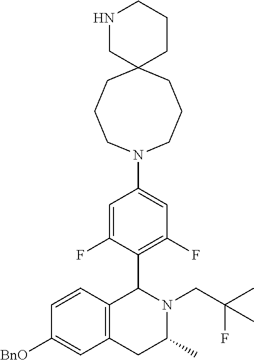 [SMILES: C[C@@H]1Cc2cc(OCc3ccccc3)ccc2C(c2c(F)cc(N3CCCC4(CCCNC4)CCC3)cc2F)N1CC(C)(C)F] | 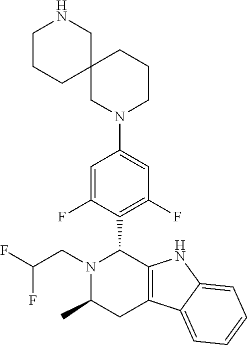 [SMILES: C[C@@H]1Cc2c([nH]c3ccccc23)[C@@H](c2c(F)cc(N3CCCC4(CCCNC4)C3)cc2F)N1CC(F)F] |
| 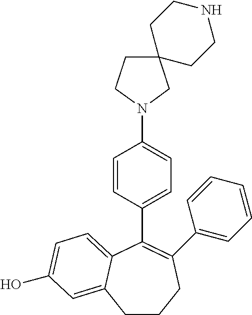 [SMILES: Oc1ccc2c(c1)CCCC(c1ccccc1)=C2c1ccc(N2CCC3(CCNCC3)C2)cc1] | 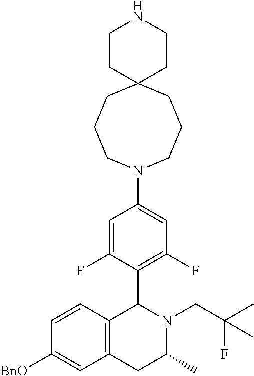 [SMILES: C[C@@H]1Cc2cc(OCc3ccccc3)ccc2C(c2c(F)cc(N3CCCC4(CCC3)CCNCC4)cc2F)N1CC(C)(C)F] | 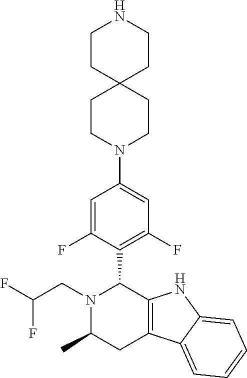 [SMILES: C[C@@H]1Cc2c([nH]c3ccccc23)[C@@H](c2c(F)cc(N3CCC4(CCNCC4)CC3)cc2F)N1CC(F)F] |
| 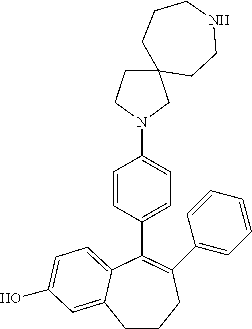 [SMILES: Oc1ccc2c(c1)CCCC(c1ccccc1)=C2c1ccc(N2CCC3(CCCNCC3)C2)cc1] | 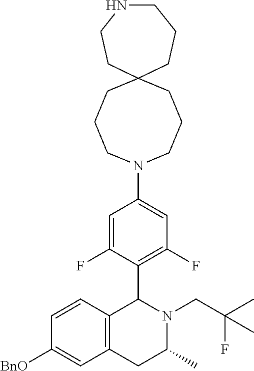 [SMILES: C[C@@H]1Cc2cc(OCc3ccccc3)ccc2C(c2c(F)cc(N3CCCC4(CCCNCC4)CCC3)cc2F)N1CC(C)(C)F] | 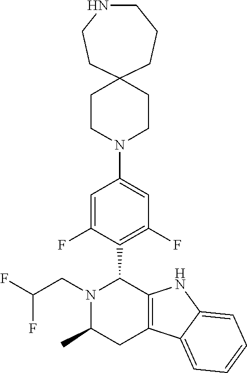 [SMILES: C[C@@H]1Cc2c([nH]c3ccccc23)[C@@H](c2c(F)cc(N3CCC4(CCCNCC4)CC3)cc2F)N1CC(F)F] |
| 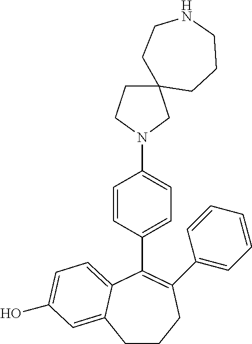 [SMILES: Oc1ccc2c(c1)CCCC(c1ccccc1)=C2c1ccc(N2CCC3(CCCNCC3)C2)cc1] | 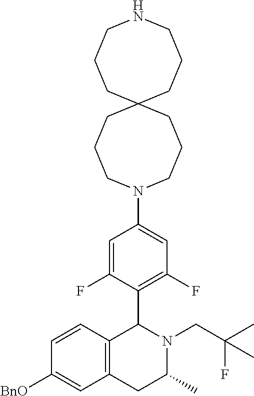 [SMILES: C[C@@H]1Cc2cc(OCc3ccccc3)ccc2C(c2c(F)cc(N3CCCC4(CCCNCCC4)CCC3)cc2F)N1CC(C)(C)F] | 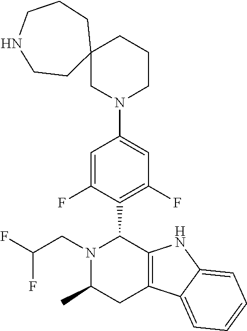 [SMILES: C[C@@H]1Cc2c([nH]c3ccccc23)[C@@H](c2c(F)cc(N3CCCC4(CCCNCC4)C3)cc2F)N1CC(F)F] |
| 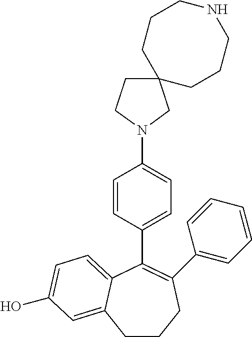 [SMILES: Oc1ccc2c(c1)CCCC(c1ccccc1)=C2c1ccc(N2CCC3(CCCNCCC3)C2)cc1] | 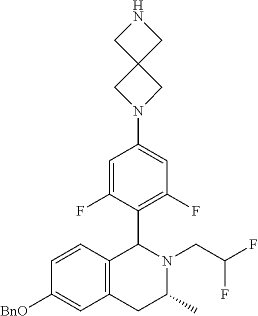 [SMILES: C[C@@H]1Cc2cc(OCc3ccccc3)ccc2C(c2c(F)cc(N3CC4(CNC4)C3)cc2F)N1CC(F)F] | 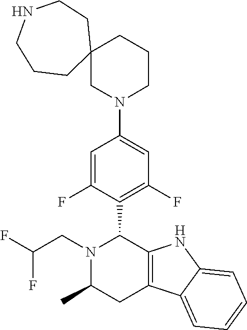 [SMILES: C[C@@H]1Cc2c([nH]c3ccccc23)[C@@H](c2c(F)cc(N3CCCC4(CCCNCC4)C3)cc2F)N1CC(F)F] |
| 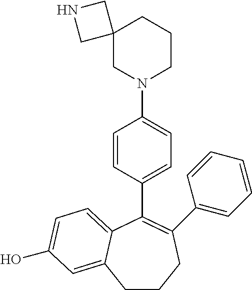 [SMILES: Oc1ccc2c(c1)CCCC(c1ccccc1)=C2c1ccc(N2CCCC3(CNC3)C2)cc1] | 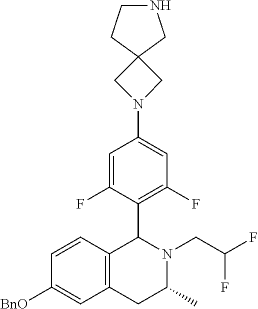 [SMILES: C[C@@H]1Cc2cc(OCc3ccccc3)ccc2C(c2c(F)cc(N3CC4(CCNC4)C3)cc2F)N1CC(F)F] | 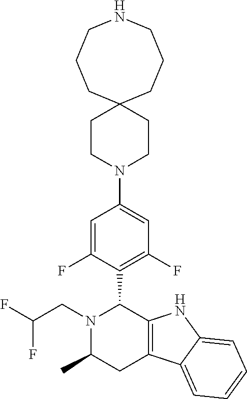 [SMILES: C[C@@H]1Cc2c([nH]c3ccccc23)[C@@H](c2c(F)cc(N3CCC4(CCCNCCC4)CC3)cc2F)N1CC(F)F] |
| 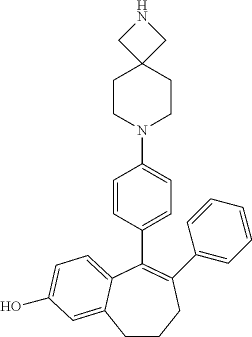 [SMILES: Oc1ccc2c(c1)CCCC(c1ccccc1)=C2c1ccc(N2CCC3(CC2)CNC3)cc1] | 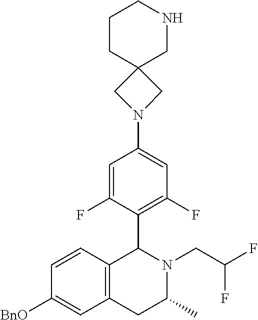 [SMILES: C[C@@H]1Cc2cc(OCc3ccccc3)ccc2C(c2c(F)cc(N3CC4(CCCNC4)C3)cc2F)N1CC(F)F] | 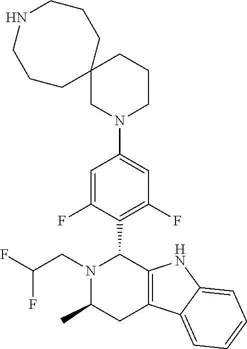 [SMILES: C[C@@H]1Cc2c([nH]c3ccccc23)[C@@H](c2c(F)cc(N3CCCC4(CCCNCCC4)C3)cc2F)N1CC(F)F] |
| 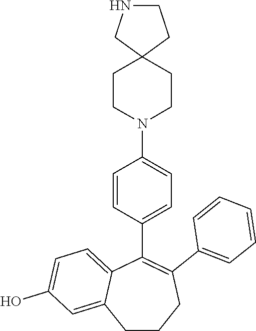 [SMILES: Oc1ccc2c(c1)CCCC(c1ccccc1)=C2c1ccc(N2CCC3(CCNC3)CC2)cc1] | 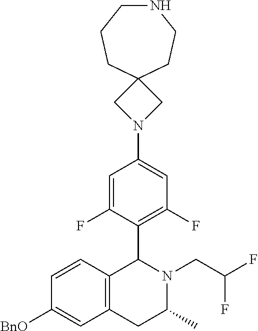 [SMILES: C[C@@H]1Cc2cc(OCc3ccccc3)ccc2C(c2c(F)cc(N3CC4(CCCNCC4)C3)cc2F)N1CC(F)F] | 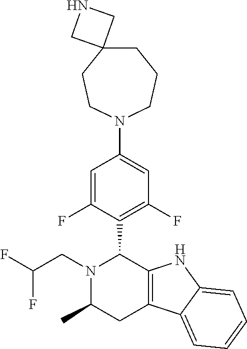 [SMILES: C[C@@H]1Cc2c([nH]c3ccccc23)[C@@H](c2c(F)cc(N3CCCC4(CC3)CNC4)cc2F)N1CC(F)F] |
| 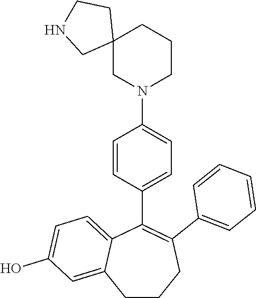 [SMILES: Oc1ccc2c(c1)CCCC(c1ccccc1)=C2c1ccc(N2CCCC3(CCNC3)C2)cc1] | 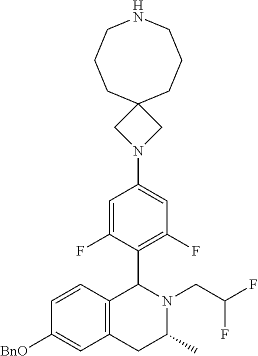 [SMILES: C[C@@H]1Cc2cc(OCc3ccccc3)ccc2C(c2c(F)cc(N3CC4(CCCNCCC4)C3)cc2F)N1CC(F)F] | 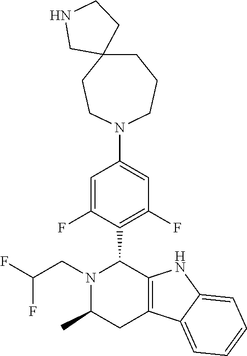 [SMILES: C[C@@H]1Cc2c([nH]c3ccccc23)[C@@H](c2c(F)cc(N3CCCC4(CCNC4)CC3)cc2F)N1CC(F)F] |
| 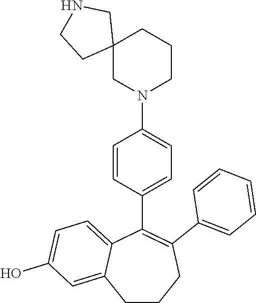 [SMILES: Oc1ccc2c(c1)CCCC(c1ccccc1)=C2c1ccc(N2CCCC3(CCNC3)C2)cc1] | 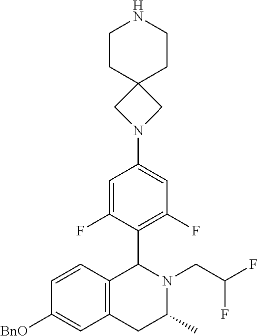 [SMILES: C[C@@H]1Cc2cc(OCc3ccccc3)ccc2C(c2c(F)cc(N3CC4(CCNCC4)C3)cc2F)N1CC(F)F] | 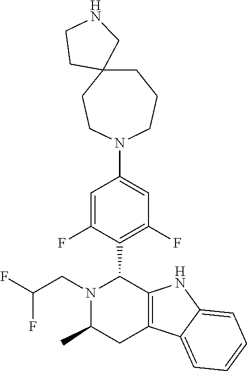 [SMILES: C[C@@H]1Cc2c([nH]c3ccccc23)[C@@H](c2c(F)cc(N3CCCC4(CCNC4)CC3)cc2F)N1CC(F)F] |
| 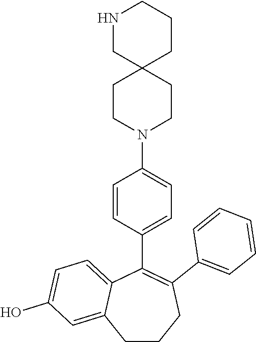 [SMILES: Oc1ccc2c(c1)CCCC(c1ccccc1)=C2c1ccc(N2CCC3(CCCNC3)CC2)cc1] | 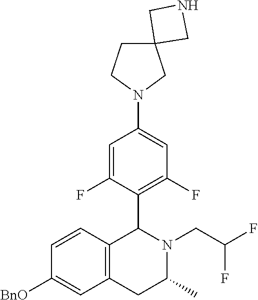 [SMILES: C[C@@H]1Cc2cc(OCc3ccccc3)ccc2C(c2c(F)cc(N3CCC4(CNC4)C3)cc2F)N1CC(F)F] | 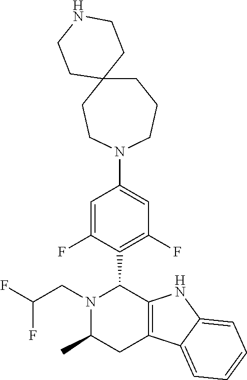 [SMILES: C[C@@H]1Cc2c([nH]c3ccccc23)[C@@H](c2c(F)cc(N3CCCC4(CCNCC4)CC3)cc2F)N1CC(F)F] |
| 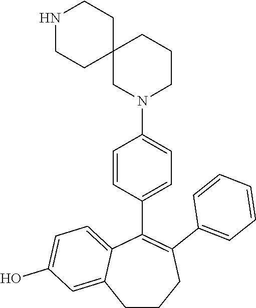 [SMILES: Oc1ccc2c(c1)CCCC(c1ccccc1)=C2c1ccc(N2CCCC3(CCNCC3)C2)cc1] | 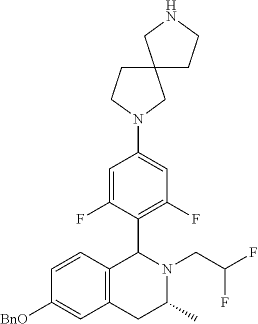 [SMILES: C[C@@H]1Cc2cc(OCc3ccccc3)ccc2C(c2c(F)cc(N3CCC4(CCNC4)C3)cc2F)N1CC(F)F] | 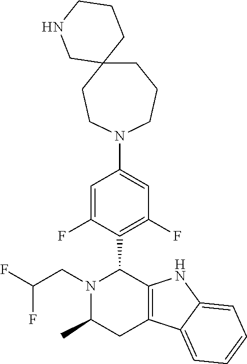 [SMILES: C[C@@H]1Cc2c([nH]c3ccccc23)[C@@H](c2c(F)cc(N3CCCC4(CCCNC4)CC3)cc2F)N1CC(F)F] |
| 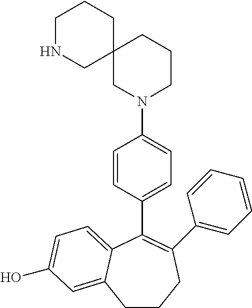 [SMILES: Oc1ccc2c(c1)CCCC(c1ccccc1)=C2c1ccc(N2CCCC3(CCCNC3)C2)cc1] | 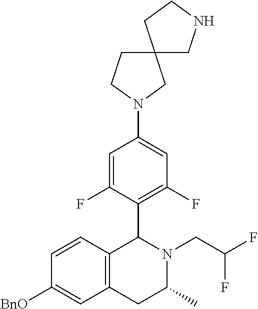 [SMILES: C[C@@H]1Cc2cc(OCc3ccccc3)ccc2C(c2c(F)cc(N3CCC4(CCNC4)C3)cc2F)N1CC(F)F] | 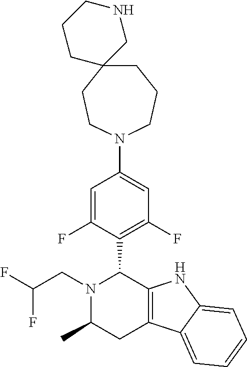 [SMILES: C[C@@H]1Cc2c([nH]c3ccccc23)[C@@H](c2c(F)cc(N3CCCC4(CCCNC4)CC3)cc2F)N1CC(F)F] |
| 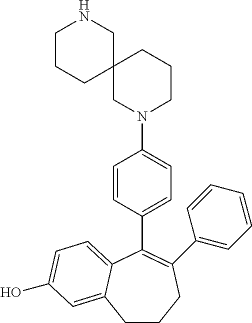 [SMILES: Oc1ccc2c(c1)CCCC(c1ccccc1)=C2c1ccc(N2CCCC3(CCCNC3)C2)cc1] | 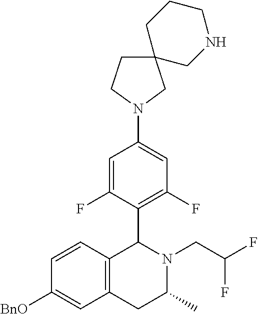 [SMILES: C[C@@H]1Cc2cc(OCc3ccccc3)ccc2C(c2c(F)cc(N3CCC4(CCCNC4)C3)cc2F)N1CC(F)F] | 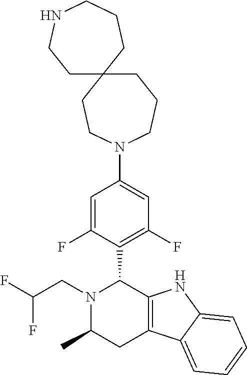 [SMILES: C[C@@H]1Cc2c([nH]c3ccccc23)[C@@H](c2c(F)cc(N3CCCC4(CCCNCC4)CC3)cc2F)N1CC(F)F] |
| 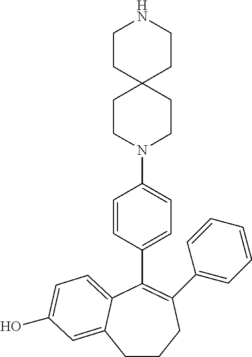 [SMILES: Oc1ccc2c(c1)CCCC(c1ccccc1)=C2c1ccc(N2CCC3(CCNCC3)CC2)cc1] | 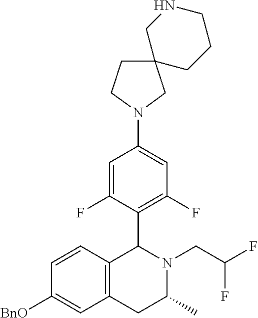 [SMILES: C[C@@H]1Cc2cc(OCc3ccccc3)ccc2C(c2c(F)cc(N3CCC4(CCCNC4)C3)cc2F)N1CC(F)F] | 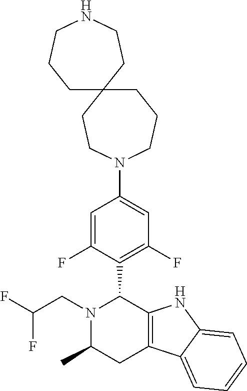 [SMILES: C[C@@H]1Cc2c([nH]c3ccccc23)[C@@H](c2c(F)cc(N3CCCC4(CCCNCC4)CC3)cc2F)N1CC(F)F] |
| 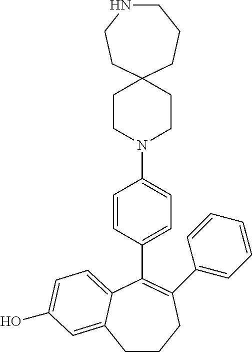 [SMILES: Oc1ccc2c(c1)CCCC(c1ccccc1)=C2c1ccc(N2CCC3(CCCNCC3)CC2)cc1] | 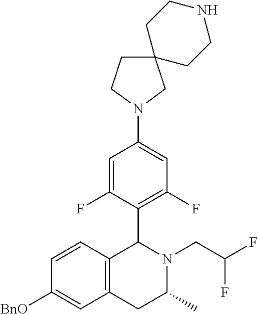 [SMILES: C[C@@H]1Cc2cc(OCc3ccccc3)ccc2C(c2c(F)cc(N3CCC4(CCNCC4)C3)cc2F)N1CC(F)F] | 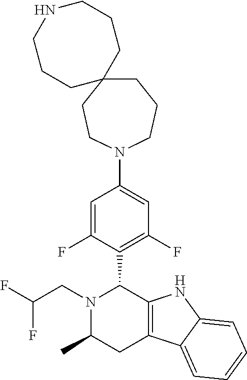 [SMILES: C[C@@H]1Cc2c([nH]c3ccccc23)[C@@H](c2c(F)cc(N3CCCC4(CCCNCCC4)CC3)cc2F)N1CC(F)F] |
| 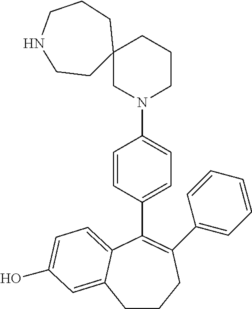 [SMILES: Oc1ccc2c(c1)CCCC(c1ccccc1)=C2c1ccc(N2CCCC3(CCCNCC3)C2)cc1] | 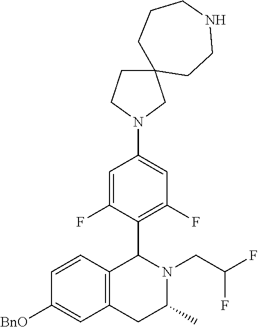 [SMILES: C[C@@H]1Cc2cc(OCc3ccccc3)ccc2C(c2c(F)cc(N3CCC4(CCCNCC4)C3)cc2F)N1CC(F)F] | 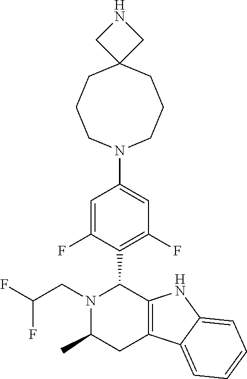 [SMILES: C[C@@H]1Cc2c([nH]c3ccccc23)[C@@H](c2c(F)cc(N3CCCC4(CCC3)CNC4)cc2F)N1CC(F)F] |
| 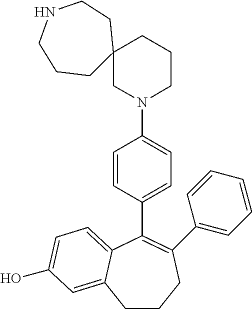 [SMILES: Oc1ccc2c(c1)CCCC(c1ccccc1)=C2c1ccc(N2CCCC3(CCCNCC3)C2)cc1] | 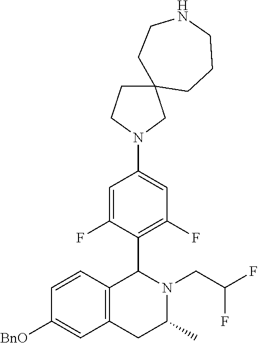 [SMILES: C[C@@H]1Cc2cc(OCc3ccccc3)ccc2C(c2c(F)cc(N3CCC4(CCCNCC4)C3)cc2F)N1CC(F)F] | 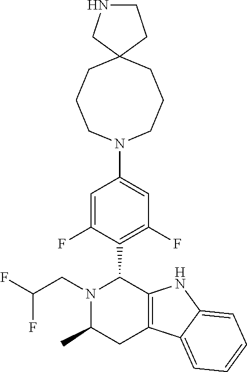 [SMILES: C[C@@H]1Cc2c([nH]c3ccccc23)[C@@H](c2c(F)cc(N3CCCC4(CCC3)CCNC4)cc2F)N1CC(F)F] |
| 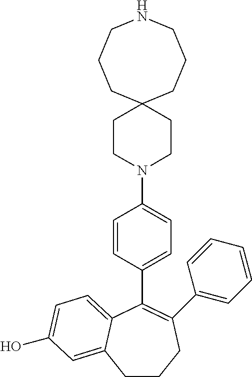 [SMILES: Oc1ccc2c(c1)CCCC(c1ccccc1)=C2c1ccc(N2CCC3(CCCNCCC3)CC2)cc1] | 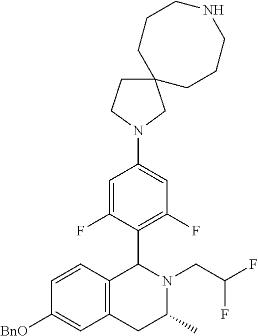 [SMILES: C[C@@H]1Cc2cc(OCc3ccccc3)ccc2C(c2c(F)cc(N3CCC4(CCCNCCC4)C3)cc2F)N1CC(F)F] | 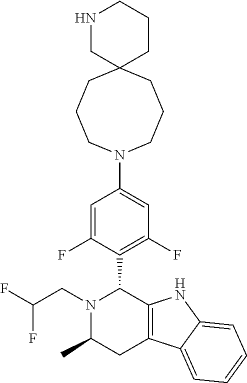 [SMILES: C[C@@H]1Cc2c([nH]c3ccccc23)[C@@H](c2c(F)cc(N3CCCC4(CCCNC4)CCC3)cc2F)N1CC(F)F] |
| 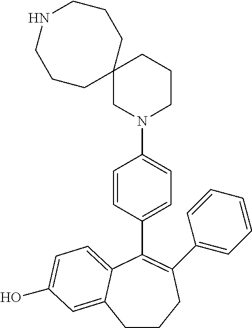 [SMILES: Oc1ccc2c(c1)CCCC(c1ccccc1)=C2c1ccc(N2CCCC3(CCCNCCC3)C2)cc1] | 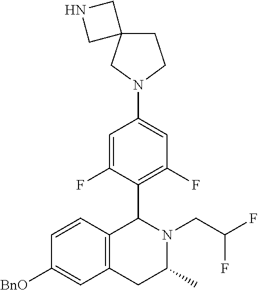 [SMILES: C[C@@H]1Cc2cc(OCc3ccccc3)ccc2C(c2c(F)cc(N3CCC4(CNC4)C3)cc2F)N1CC(F)F] | 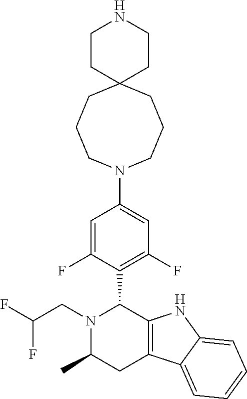 [SMILES: C[C@@H]1Cc2c([nH]c3ccccc23)[C@@H](c2c(F)cc(N3CCCC4(CCC3)CCNCC4)cc2F)N1CC(F)F] |
| 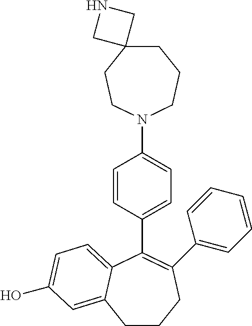 [SMILES: Oc1ccc2c(c1)CCCC(c1ccccc1)=C2c1ccc(N2CCCC3(CC2)CNC3)cc1] | 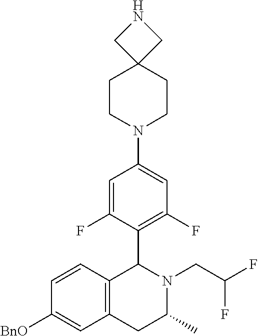 [SMILES: C[C@@H]1Cc2cc(OCc3ccccc3)ccc2C(c2c(F)cc(N3CCC4(CC3)CNC4)cc2F)N1CC(F)F] | 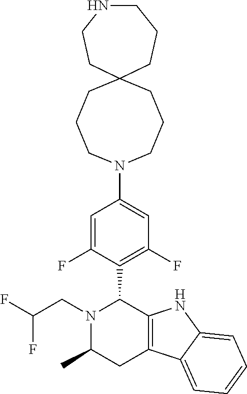 [SMILES: C[C@@H]1Cc2c([nH]c3ccccc23)[C@@H](c2c(F)cc(N3CCCC4(CCCNCC4)CCC3)cc2F)N1CC(F)F] |
| 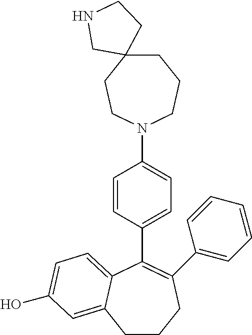 [SMILES: Oc1ccc2c(c1)CCCC(c1ccccc1)=C2c1ccc(N2CCCC3(CCNC3)CC2)cc1] | 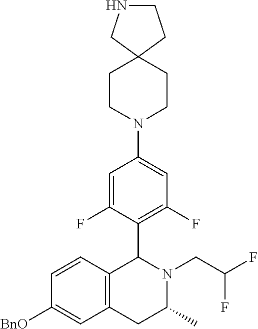 [SMILES: C[C@@H]1Cc2cc(OCc3ccccc3)ccc2C(c2c(F)cc(N3CCC4(CCNC4)CC3)cc2F)N1CC(F)F] | 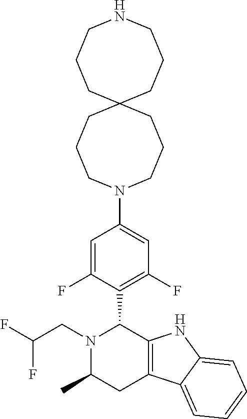 [SMILES: C[C@@H]1Cc2c([nH]c3ccccc23)[C@@H](c2c(F)cc(N3CCCC4(CCCNCCC4)CCC3)cc2F)N1CC(F)F] |
| 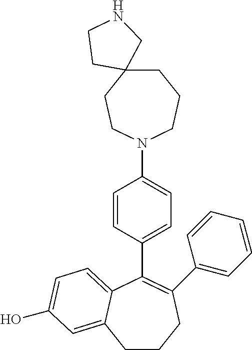 [SMILES: Oc1ccc2c(c1)CCCC(c1ccccc1)=C2c1ccc(N2CCCC3(CCNC3)CC2)cc1] | 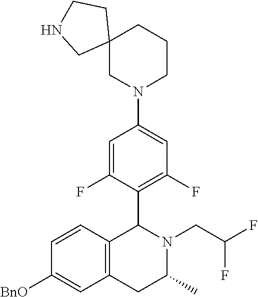 [SMILES: C[C@@H]1Cc2cc(OCc3ccccc3)ccc2C(c2c(F)cc(N3CCCC4(CCNC4)C3)cc2F)N1CC(F)F] | 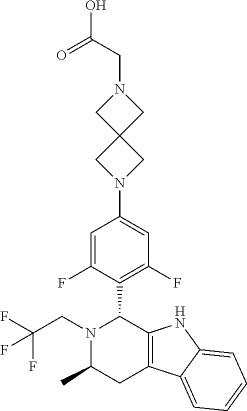 [SMILES: C[C@@H]1Cc2c([nH]c3ccccc23)[C@@H](c2c(F)cc(N3CC4(CN(CC(=O)O)C4)C3)cc2F)N1CC(F)(F)F] |
| 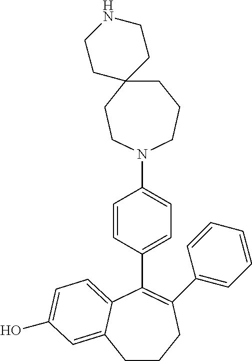 [SMILES: Oc1ccc2c(c1)CCCC(c1ccccc1)=C2c1ccc(N2CCCC3(CCNCC3)CC2)cc1] | 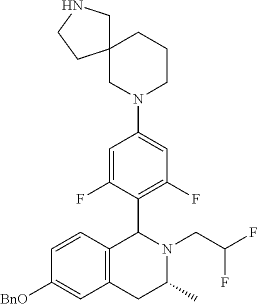 [SMILES: C[C@@H]1Cc2cc(OCc3ccccc3)ccc2C(c2c(F)cc(N3CCCC4(CCNC4)C3)cc2F)N1CC(F)F] | 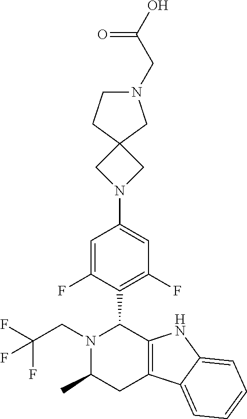 [SMILES: C[C@@H]1Cc2c([nH]c3ccccc23)[C@@H](c2c(F)cc(N3CC4(CCN(CC(=O)O)C4)C3)cc2F)N1CC(F)(F)F] |
| 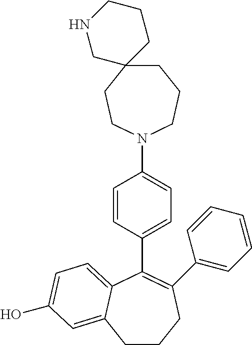 [SMILES: Oc1ccc2c(c1)CCCC(c1ccccc1)=C2c1ccc(N2CCCC3(CCCNC3)CC2)cc1] | 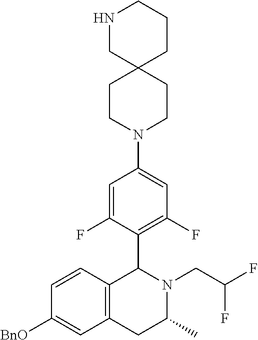 [SMILES: C[C@@H]1Cc2cc(OCc3ccccc3)ccc2C(c2c(F)cc(N3CCC4(CCCNC4)CC3)cc2F)N1CC(F)F] | 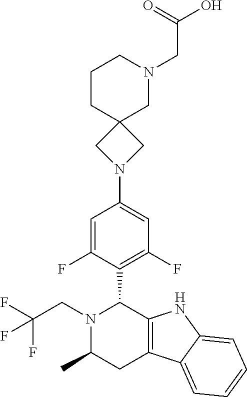 [SMILES: C[C@@H]1Cc2c([nH]c3ccccc23)[C@@H](c2c(F)cc(N3CC4(CCCN(CC(=O)O)C4)C3)cc2F)N1CC(F)(F)F] |
| 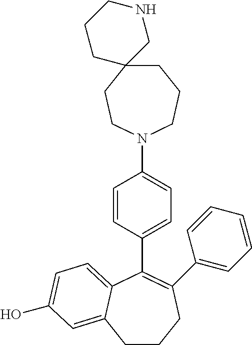 [SMILES: Oc1ccc2c(c1)CCCC(c1ccccc1)=C2c1ccc(N2CCCC3(CCCNC3)CC2)cc1] | 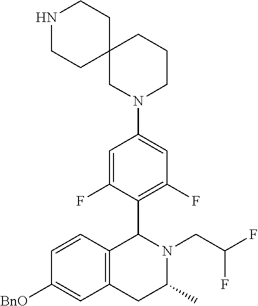 [SMILES: C[C@@H]1Cc2cc(OCc3ccccc3)ccc2C(c2c(F)cc(N3CCCC4(CCNCC4)C3)cc2F)N1CC(F)F] | 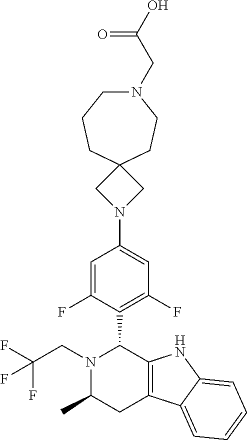 [SMILES: C[C@@H]1Cc2c([nH]c3ccccc23)[C@@H](c2c(F)cc(N3CC4(CCCN(CC(=O)O)CC4)C3)cc2F)N1CC(F)(F)F] |
| 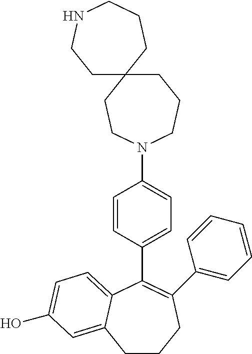 [SMILES: Oc1ccc2c(c1)CCCC(c1ccccc1)=C2c1ccc(N2CCCC3(CCCNCC3)CC2)cc1] | 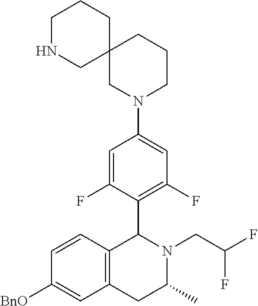 [SMILES: C[C@@H]1Cc2cc(OCc3ccccc3)ccc2C(c2c(F)cc(N3CCCC4(CCCNC4)C3)cc2F)N1CC(F)F] | 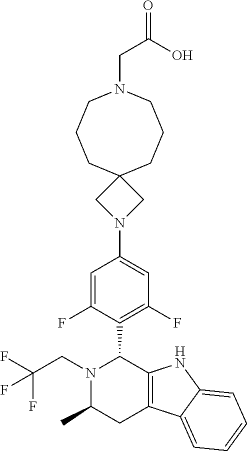 [SMILES: C[C@@H]1Cc2c([nH]c3ccccc23)[C@@H](c2c(F)cc(N3CC4(CCCN(CC(=O)O)CCC4)C3)cc2F)N1CC(F)(F)F] |
| 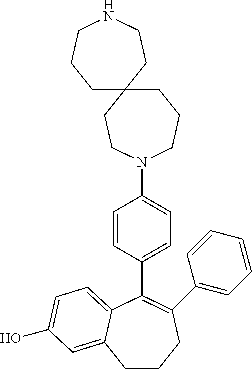 [SMILES: Oc1ccc2c(c1)CCCC(c1ccccc1)=C2c1ccc(N2CCCC3(CCCNCC3)CC2)cc1] | 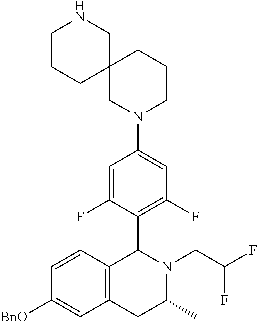 [SMILES: C[C@@H]1Cc2cc(OCc3ccccc3)ccc2C(c2c(F)cc(N3CCCC4(CCCNC4)C3)cc2F)N1CC(F)F] | 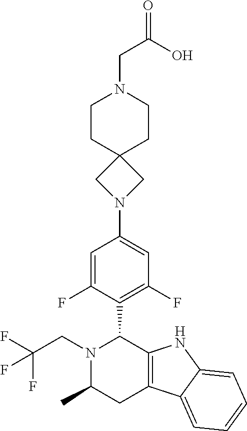 [SMILES: C[C@@H]1Cc2c([nH]c3ccccc23)[C@@H](c2c(F)cc(N3CC4(CCN(CC(=O)O)CC4)C3)cc2F)N1CC(F)(F)F] |
| 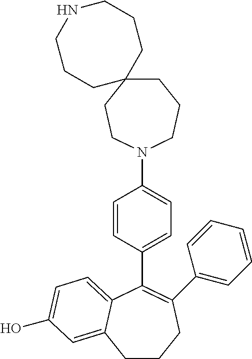 [SMILES: Oc1ccc2c(c1)CCCC(c1ccccc1)=C2c1ccc(N2CCCC3(CCCNCCC3)CC2)cc1] | 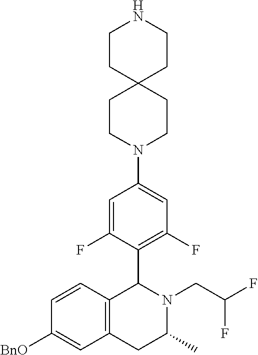 [SMILES: C[C@@H]1Cc2cc(OCc3ccccc3)ccc2C(c2c(F)cc(N3CCC4(CCNCC4)CC3)cc2F)N1CC(F)F] | 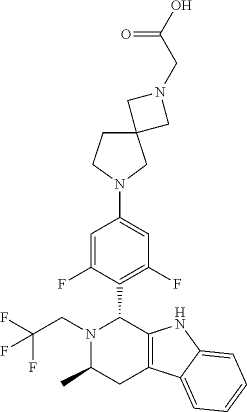 [SMILES: C[C@@H]1Cc2c([nH]c3ccccc23)[C@@H](c2c(F)cc(N3CCC4(CN(CC(=O)O)C4)C3)cc2F)N1CC(F)(F)F] |
| 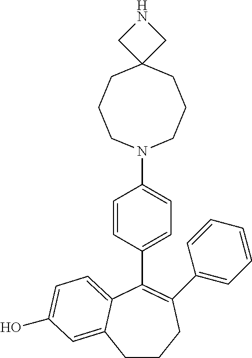 [SMILES: Oc1ccc2c(c1)CCCC(c1ccccc1)=C2c1ccc(N2CCCC3(CCC2)CNC3)cc1] | 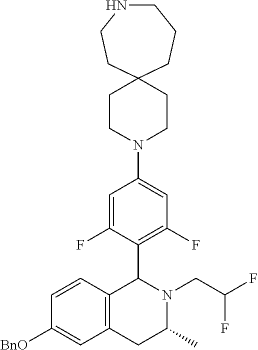 [SMILES: C[C@@H]1Cc2cc(OCc3ccccc3)ccc2C(c2c(F)cc(N3CCC4(CCCNCC4)CC3)cc2F)N1CC(F)F] | 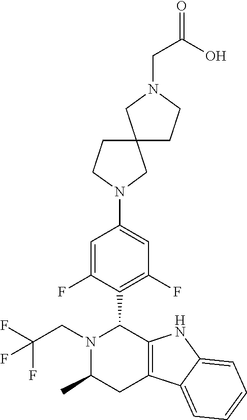 [SMILES: C[C@@H]1Cc2c([nH]c3ccccc23)[C@@H](c2c(F)cc(N3CCC4(CCN(CC(=O)O)C4)C3)cc2F)N1CC(F)(F)F] |
| 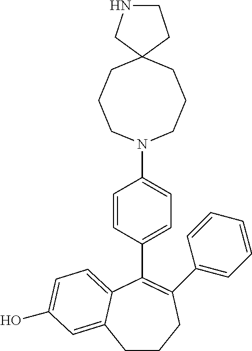 [SMILES: Oc1ccc2c(c1)CCCC(c1ccccc1)=C2c1ccc(N2CCCC3(CCC2)CCNC3)cc1] | 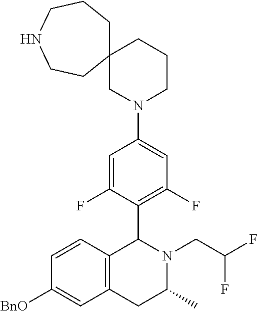 [SMILES: C[C@@H]1Cc2cc(OCc3ccccc3)ccc2C(c2c(F)cc(N3CCCC4(CCCNCC4)C3)cc2F)N1CC(F)F] | 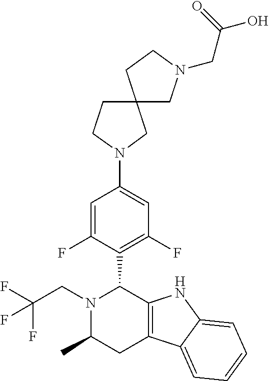 [SMILES: C[C@@H]1Cc2c([nH]c3ccccc23)[C@@H](c2c(F)cc(N3CCC4(CCN(CC(=O)O)C4)C3)cc2F)N1CC(F)(F)F] |
| 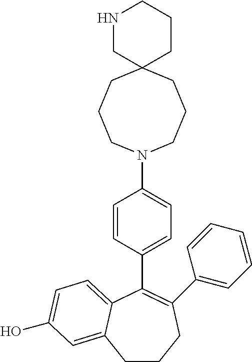 [SMILES: Oc1ccc2c(c1)CCCC(c1ccccc1)=C2c1ccc(N2CCCC3(CCCNC3)CCC2)cc1] | 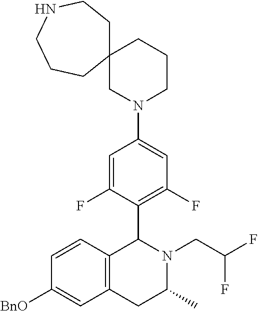 [SMILES: C[C@@H]1Cc2cc(OCc3ccccc3)ccc2C(c2c(F)cc(N3CCCC4(CCCNCC4)C3)cc2F)N1CC(F)F] | 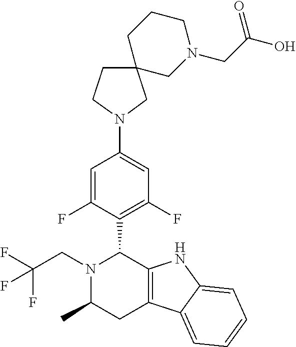 [SMILES: C[C@@H]1Cc2c([nH]c3ccccc23)[C@@H](c2c(F)cc(N3CCC4(CCCN(CC(=O)O)C4)C3)cc2F)N1CC(F)(F)F] |
| 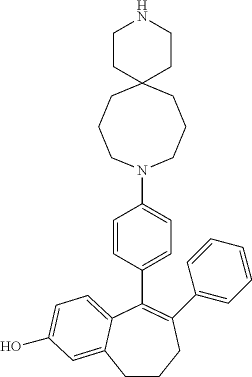 [SMILES: Oc1ccc2c(c1)CCCC(c1ccccc1)=C2c1ccc(N2CCCC3(CCC2)CCNCC3)cc1] | 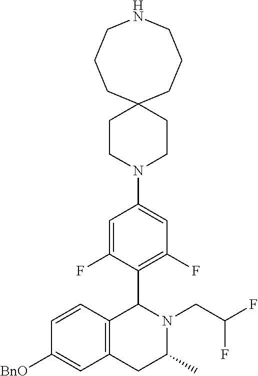 [SMILES: C[C@@H]1Cc2cc(OCc3ccccc3)ccc2C(c2c(F)cc(N3CCC4(CCCNCCC4)CC3)cc2F)N1CC(F)F] | 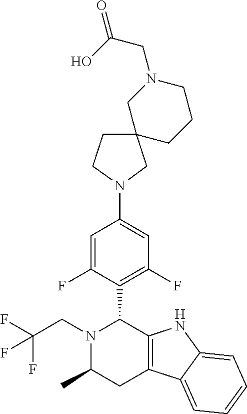 [SMILES: C[C@@H]1Cc2c([nH]c3ccccc23)[C@@H](c2c(F)cc(N3CCC4(CCCN(CC(=O)O)C4)C3)cc2F)N1CC(F)(F)F] |
| 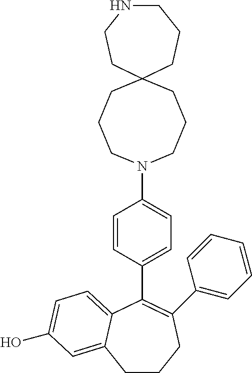 [SMILES: Oc1ccc2c(c1)CCCC(c1ccccc1)=C2c1ccc(N2CCCC3(CCCNCC3)CCC2)cc1] | 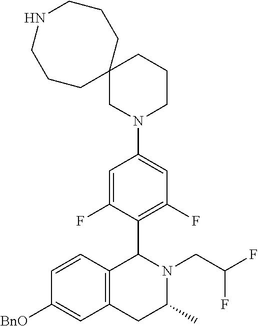 [SMILES: C[C@@H]1Cc2cc(OCc3ccccc3)ccc2C(c2c(F)cc(N3CCCC4(CCCNCCC4)C3)cc2F)N1CC(F)F] | 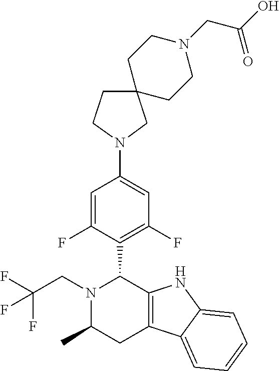 [SMILES: C[C@@H]1Cc2c([nH]c3ccccc23)[C@@H](c2c(F)cc(N3CCC4(CCN(CC(=O)O)CC4)C3)cc2F)N1CC(F)(F)F] |
| 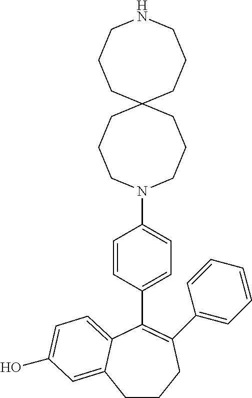 [SMILES: Oc1ccc2c(c1)CCCC(c1ccccc1)=C2c1ccc(N2CCCC3(CCCNCCC3)CCC2)cc1] | 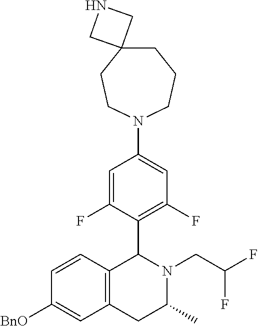 [SMILES: C[C@@H]1Cc2cc(OCc3ccccc3)ccc2C(c2c(F)cc(N3CCCC4(CC3)CNC4)cc2F)N1CC(F)F] | 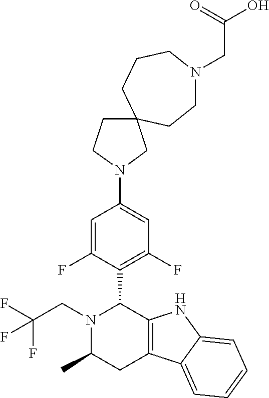 [SMILES: C[C@@H]1Cc2c([nH]c3ccccc23)[C@@H](c2c(F)cc(N3CCC4(CCCN(CC(=O)O)CC4)C3)cc2F)N1CC(F)(F)F] |
| 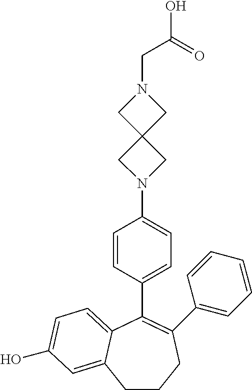 [SMILES: O=C(O)CN1CC2(C1)CN(c1ccc(C3=C(c4ccccc4)CCCc4cc(O)ccc43)cc1)C2] | 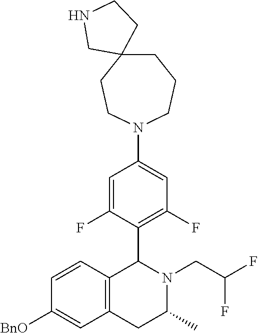 [SMILES: C[C@@H]1Cc2cc(OCc3ccccc3)ccc2C(c2c(F)cc(N3CCCC4(CCNC4)CC3)cc2F)N1CC(F)F] | 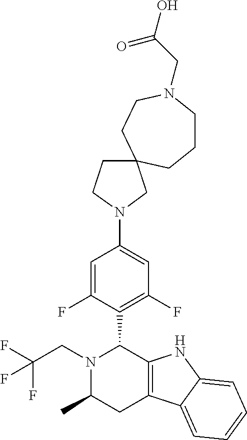 [SMILES: C[C@@H]1Cc2c([nH]c3ccccc23)[C@@H](c2c(F)cc(N3CCC4(CCCN(CC(=O)O)CC4)C3)cc2F)N1CC(F)(F)F] |
| 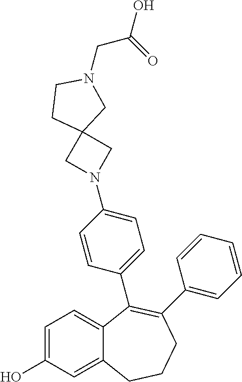 [SMILES: O=C(O)CN1CCC2(C1)CN(c1ccc(C3=C(c4ccccc4)CCCc4cc(O)ccc43)cc1)C2] | 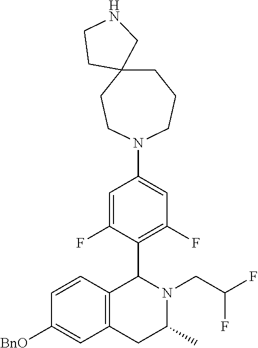 [SMILES: C[C@@H]1Cc2cc(OCc3ccccc3)ccc2C(c2c(F)cc(N3CCCC4(CCNC4)CC3)cc2F)N1CC(F)F] | 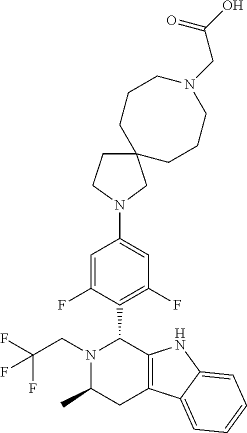 [SMILES: C[C@@H]1Cc2c([nH]c3ccccc23)[C@@H](c2c(F)cc(N3CCC4(CCCN(CC(=O)O)CCC4)C3)cc2F)N1CC(F)(F)F] |
| 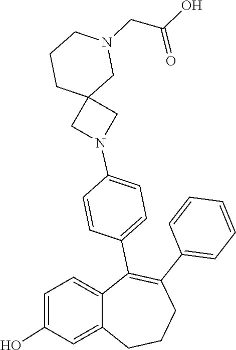 [SMILES: O=C(O)CN1CCCC2(C1)CN(c1ccc(C3=C(c4ccccc4)CCCc4cc(O)ccc43)cc1)C2] | 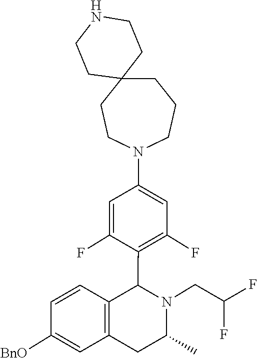 [SMILES: C[C@@H]1Cc2cc(OCc3ccccc3)ccc2C(c2c(F)cc(N3CCCC4(CCNCC4)CC3)cc2F)N1CC(F)F] | 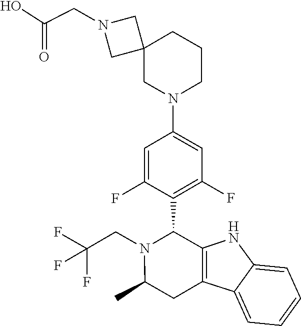 [SMILES: C[C@@H]1Cc2c([nH]c3ccccc23)[C@@H](c2c(F)cc(N3CCCC4(CN(CC(=O)O)C4)C3)cc2F)N1CC(F)(F)F] |
| 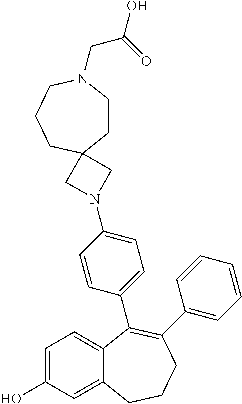 [SMILES: O=C(O)CN1CCCC2(CC1)CN(c1ccc(C3=C(c4ccccc4)CCCc4cc(O)ccc43)cc1)C2] | 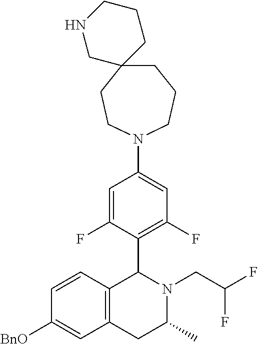 [SMILES: C[C@@H]1Cc2cc(OCc3ccccc3)ccc2C(c2c(F)cc(N3CCCC4(CCCNC4)CC3)cc2F)N1CC(F)F] | 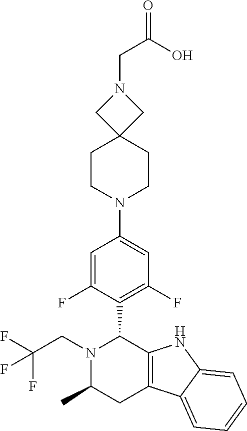 [SMILES: C[C@@H]1Cc2c([nH]c3ccccc23)[C@@H](c2c(F)cc(N3CCC4(CC3)CN(CC(=O)O)C4)cc2F)N1CC(F)(F)F] |
| 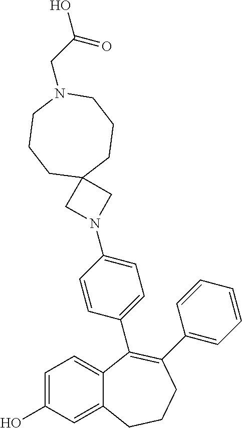 [SMILES: O=C(O)CN1CCCC2(CCC1)CN(c1ccc(C3=C(c4ccccc4)CCCc4cc(O)ccc43)cc1)C2] | 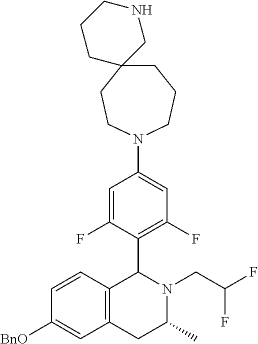 [SMILES: C[C@@H]1Cc2cc(OCc3ccccc3)ccc2C(c2c(F)cc(N3CCCC4(CCCNC4)CC3)cc2F)N1CC(F)F] | 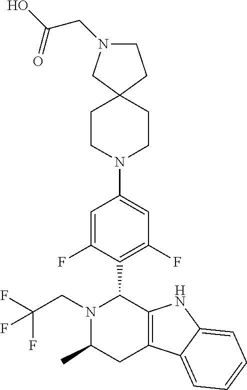 [SMILES: C[C@@H]1Cc2c([nH]c3ccccc23)[C@@H](c2c(F)cc(N3CCC4(CCN(CC(=O)O)C4)CC3)cc2F)N1CC(F)(F)F] |
| 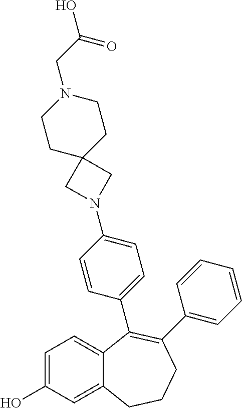 [SMILES: O=C(O)CN1CCC2(CC1)CN(c1ccc(C3=C(c4ccccc4)CCCc4cc(O)ccc43)cc1)C2] | 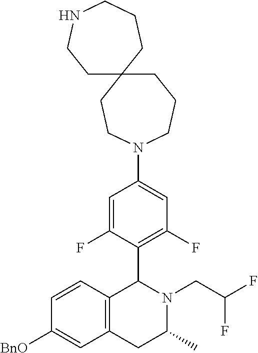 [SMILES: C[C@@H]1Cc2cc(OCc3ccccc3)ccc2C(c2c(F)cc(N3CCCC4(CCCNCC4)CC3)cc2F)N1CC(F)F] | 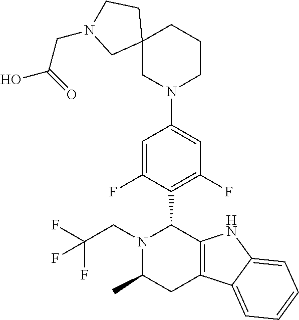 [SMILES: C[C@@H]1Cc2c([nH]c3ccccc23)[C@@H](c2c(F)cc(N3CCCC4(CCN(CC(=O)O)C4)C3)cc2F)N1CC(F)(F)F] |
| 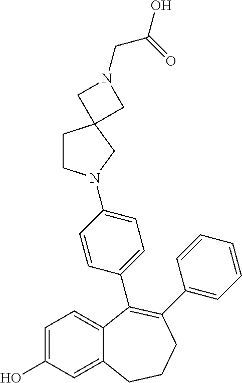 [SMILES: O=C(O)CN1CC2(CCN(c3ccc(C4=C(c5ccccc5)CCCc5cc(O)ccc54)cc3)C2)C1] | 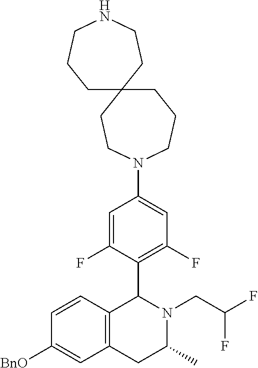 [SMILES: C[C@@H]1Cc2cc(OCc3ccccc3)ccc2C(c2c(F)cc(N3CCCC4(CCCNCC4)CC3)cc2F)N1CC(F)F] | 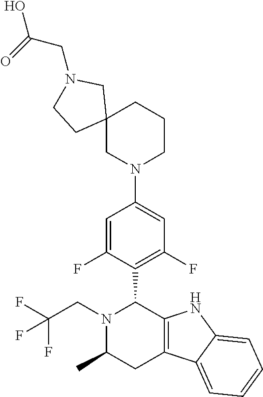 [SMILES: C[C@@H]1Cc2c([nH]c3ccccc23)[C@@H](c2c(F)cc(N3CCCC4(CCN(CC(=O)O)C4)C3)cc2F)N1CC(F)(F)F] |
| 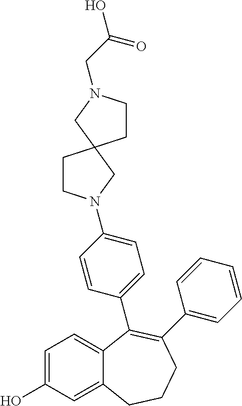 [SMILES: O=C(O)CN1CCC2(CCN(c3ccc(C4=C(c5ccccc5)CCCc5cc(O)ccc54)cc3)C2)C1] | 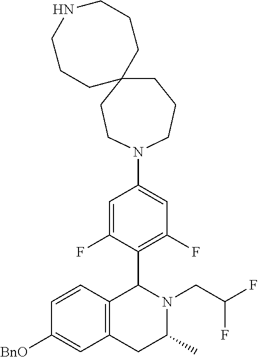 [SMILES: C[C@@H]1Cc2cc(OCc3ccccc3)ccc2C(c2c(F)cc(N3CCCC4(CCCNCCC4)CC3)cc2F)N1CC(F)F] | 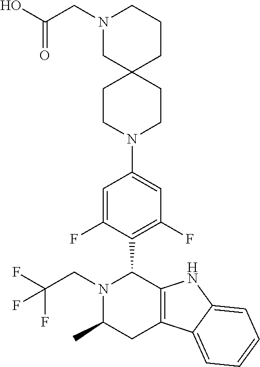 [SMILES: C[C@@H]1Cc2c([nH]c3ccccc23)[C@@H](c2c(F)cc(N3CCC4(CCCN(CC(=O)O)C4)CC3)cc2F)N1CC(F)(F)F] |
| 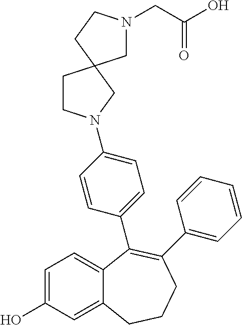 [SMILES: O=C(O)CN1CCC2(CCN(c3ccc(C4=C(c5ccccc5)CCCc5cc(O)ccc54)cc3)C2)C1] | 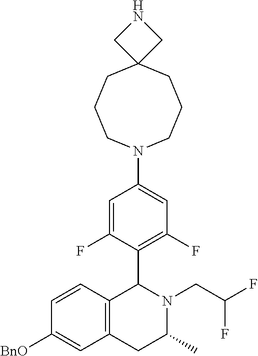 [SMILES: C[C@@H]1Cc2cc(OCc3ccccc3)ccc2C(c2c(F)cc(N3CCCC4(CCC3)CNC4)cc2F)N1CC(F)F] | 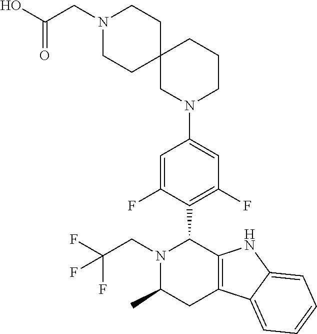 [SMILES: C[C@@H]1Cc2c([nH]c3ccccc23)[C@@H](c2c(F)cc(N3CCCC4(CCN(CC(=O)O)CC4)C3)cc2F)N1CC(F)(F)F] |
| 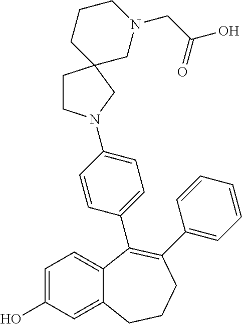 [SMILES: O=C(O)CN1CCCC2(CCN(c3ccc(C4=C(c5ccccc5)CCCc5cc(O)ccc54)cc3)C2)C1] | 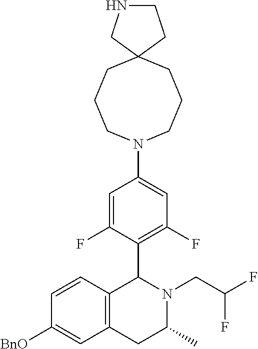 [SMILES: C[C@@H]1Cc2cc(OCc3ccccc3)ccc2C(c2c(F)cc(N3CCCC4(CCC3)CCNC4)cc2F)N1CC(F)F] | 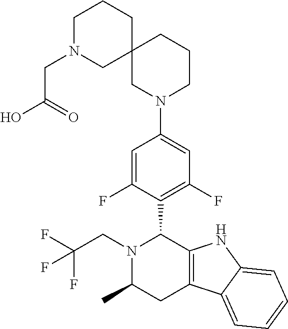 [SMILES: C[C@@H]1Cc2c([nH]c3ccccc23)[C@@H](c2c(F)cc(N3CCCC4(CCCN(CC(=O)O)C4)C3)cc2F)N1CC(F)(F)F] |
| 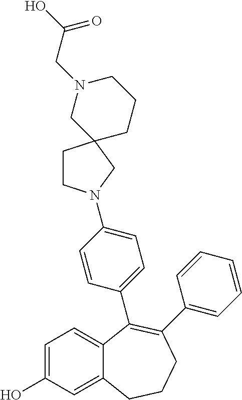 [SMILES: O=C(O)CN1CCCC2(CCN(c3ccc(C4=C(c5ccccc5)CCCc5cc(O)ccc54)cc3)C2)C1] | 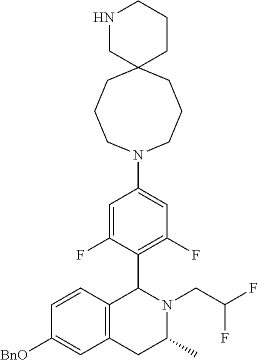 [SMILES: C[C@@H]1Cc2cc(OCc3ccccc3)ccc2C(c2c(F)cc(N3CCCC4(CCCNC4)CCC3)cc2F)N1CC(F)F] | 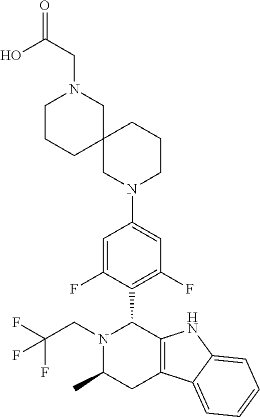 [SMILES: C[C@@H]1Cc2c([nH]c3ccccc23)[C@@H](c2c(F)cc(N3CCCC4(CCCN(CC(=O)O)C4)C3)cc2F)N1CC(F)(F)F] |
| 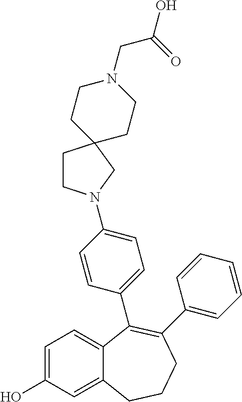 [SMILES: O=C(O)CN1CCC2(CC1)CCN(c1ccc(C3=C(c4ccccc4)CCCc4cc(O)ccc43)cc1)C2] | 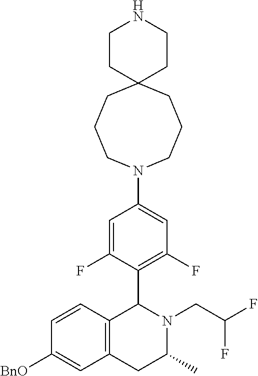 [SMILES: C[C@@H]1Cc2cc(OCc3ccccc3)ccc2C(c2c(F)cc(N3CCCC4(CCC3)CCNCC4)cc2F)N1CC(F)F] | 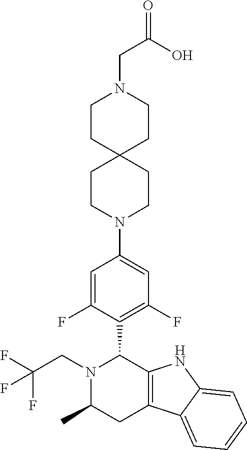 [SMILES: C[C@@H]1Cc2c([nH]c3ccccc23)[C@@H](c2c(F)cc(N3CCC4(CCN(CC(=O)O)CC4)CC3)cc2F)N1CC(F)(F)F] |
| 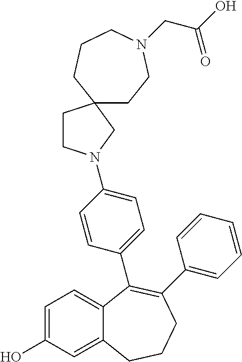 [SMILES: O=C(O)CN1CCCC2(CC1)CCN(c1ccc(C3=C(c4ccccc4)CCCc4cc(O)ccc43)cc1)C2] | 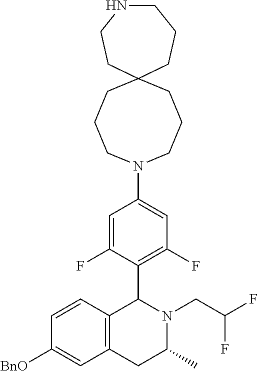 [SMILES: C[C@@H]1Cc2cc(OCc3ccccc3)ccc2C(c2c(F)cc(N3CCCC4(CCCNCC4)CCC3)cc2F)N1CC(F)F] | 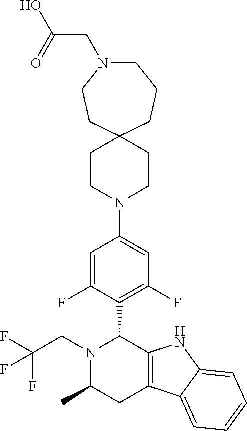 [SMILES: C[C@@H]1Cc2c([nH]c3ccccc23)[C@@H](c2c(F)cc(N3CCC4(CCCN(CC(=O)O)CC4)CC3)cc2F)N1CC(F)(F)F] |
| 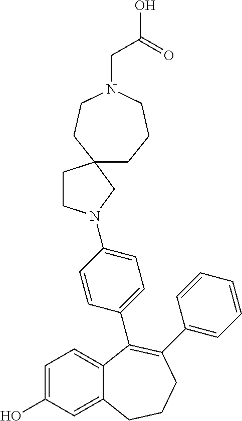 [SMILES: O=C(O)CN1CCCC2(CC1)CCN(c1ccc(C3=C(c4ccccc4)CCCc4cc(O)ccc43)cc1)C2] | 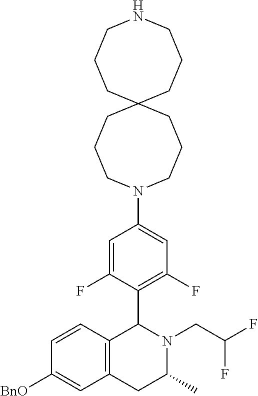 [SMILES: C[C@@H]1Cc2cc(OCc3ccccc3)ccc2C(c2c(F)cc(N3CCCC4(CCCNCCC4)CCC3)cc2F)N1CC(F)F] | 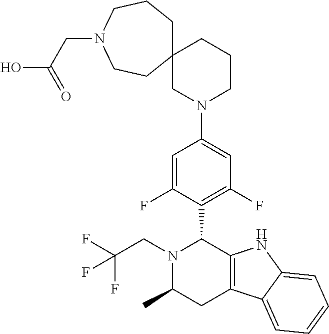 [SMILES: C[C@@H]1Cc2c([nH]c3ccccc23)[C@@H](c2c(F)cc(N3CCCC4(CCCN(CC(=O)O)CC4)C3)cc2F)N1CC(F)(F)F] |
| 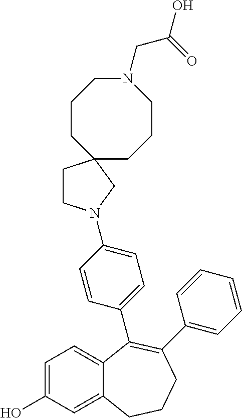 [SMILES: O=C(O)CN1CCCC2(CCC1)CCN(c1ccc(C3=C(c4ccccc4)CCCc4cc(O)ccc43)cc1)C2] | 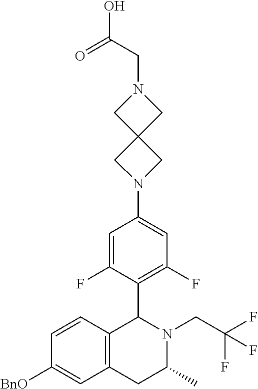 [SMILES: C[C@@H]1Cc2cc(OCc3ccccc3)ccc2C(c2c(F)cc(N3CC4(CN(CC(=O)O)C4)C3)cc2F)N1CC(F)(F)F] | 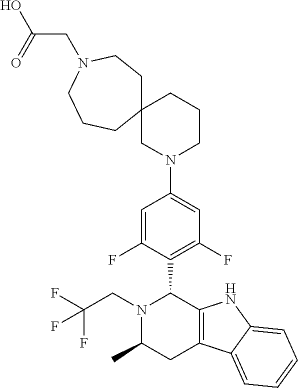 [SMILES: C[C@@H]1Cc2c([nH]c3ccccc23)[C@@H](c2c(F)cc(N3CCCC4(CCCN(CC(=O)O)CC4)C3)cc2F)N1CC(F)(F)F] |
| 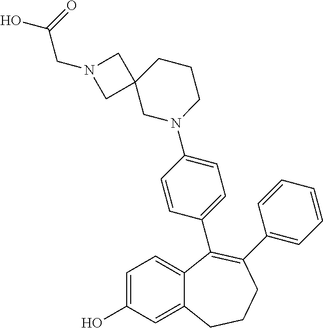 [SMILES: O=C(O)CN1CC2(CCCN(c3ccc(C4=C(c5ccccc5)CCCc5cc(O)ccc54)cc3)C2)C1] | 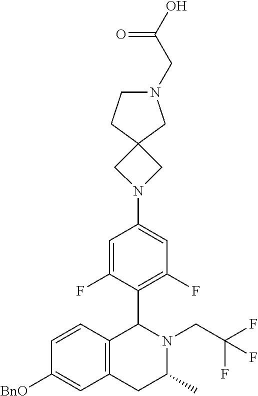 [SMILES: C[C@@H]1Cc2cc(OCc3ccccc3)ccc2C(c2c(F)cc(N3CC4(CCN(CC(=O)O)C4)C3)cc2F)N1CC(F)(F)F] | 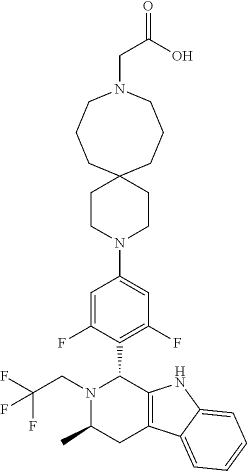 [SMILES: C[C@@H]1Cc2c([nH]c3ccccc23)[C@@H](c2c(F)cc(N3CCC4(CCCN(CC(=O)O)CCC4)CC3)cc2F)N1CC(F)(F)F] |
| 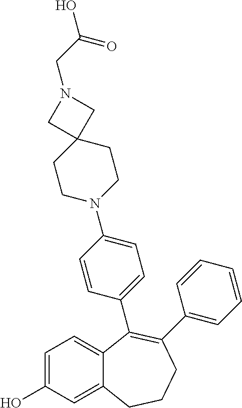 [SMILES: O=C(O)CN1CC2(CCN(c3ccc(C4=C(c5ccccc5)CCCc5cc(O)ccc54)cc3)CC2)C1] | 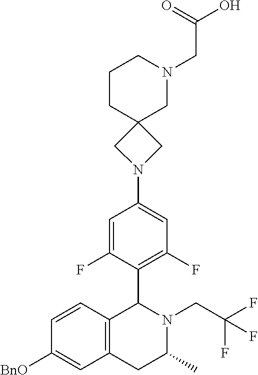 [SMILES: C[C@@H]1Cc2cc(OCc3ccccc3)ccc2C(c2c(F)cc(N3CC4(CCCN(CC(=O)O)C4)C3)cc2F)N1CC(F)(F)F] | 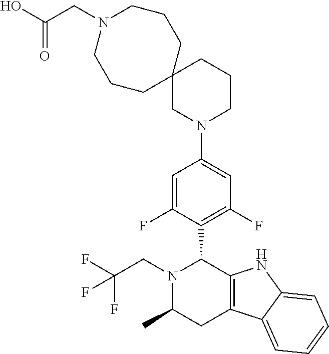 [SMILES: C[C@@H]1Cc2c([nH]c3ccccc23)[C@@H](c2c(F)cc(N3CCCC4(CCCN(CC(=O)O)CCC4)C3)cc2F)N1CC(F)(F)F] |
| 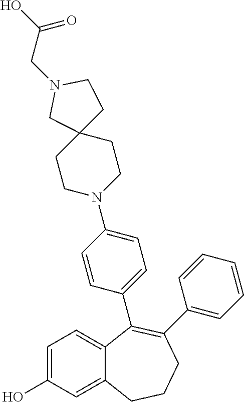 [SMILES: O=C(O)CN1CCC2(CCN(c3ccc(C4=C(c5ccccc5)CCCc5cc(O)ccc54)cc3)CC2)C1] | 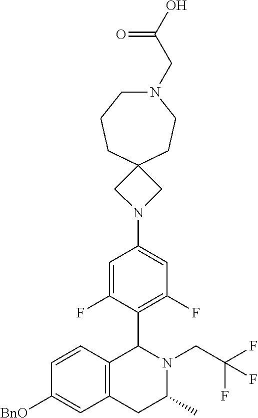 [SMILES: C[C@@H]1Cc2cc(OCc3ccccc3)ccc2C(c2c(F)cc(N3CC4(CCCN(CC(=O)O)CC4)C3)cc2F)N1CC(F)(F)F] | 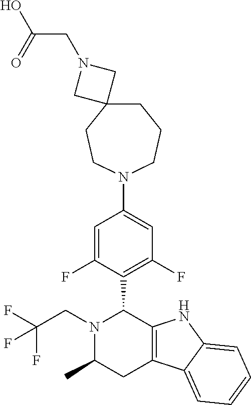 [SMILES: C[C@@H]1Cc2c([nH]c3ccccc23)[C@@H](c2c(F)cc(N3CCCC4(CC3)CN(CC(=O)O)C4)cc2F)N1CC(F)(F)F] |
| 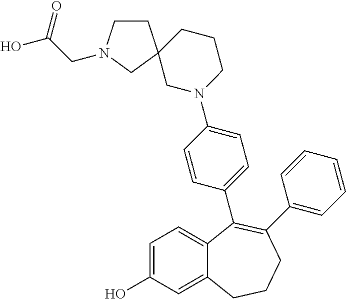 [SMILES: O=C(O)CN1CCC2(CCCN(c3ccc(C4=C(c5ccccc5)CCCc5cc(O)ccc54)cc3)C2)C1] | 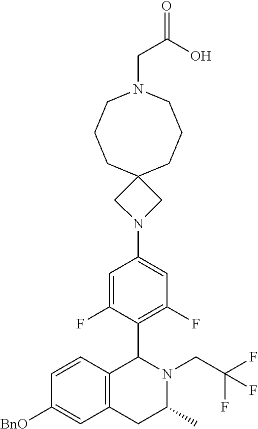 [SMILES: C[C@@H]1Cc2cc(OCc3ccccc3)ccc2C(c2c(F)cc(N3CC4(CCCN(CC(=O)O)CCC4)C3)cc2F)N1CC(F)(F)F] | 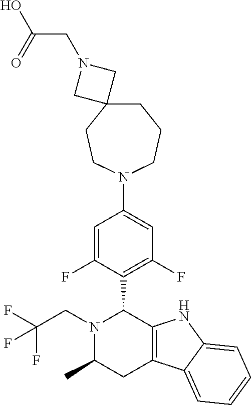 [SMILES: C[C@@H]1Cc2c([nH]c3ccccc23)[C@@H](c2c(F)cc(N3CCCC4(CC3)CN(CC(=O)O)C4)cc2F)N1CC(F)(F)F] |
| 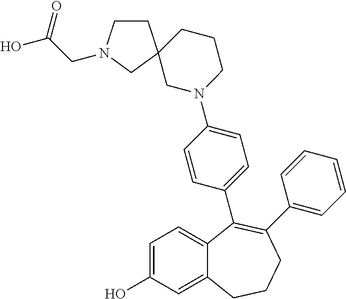 [SMILES: O=C(O)CN1CCC2(CCCN(c3ccc(C4=C(c5ccccc5)CCCc5cc(O)ccc54)cc3)C2)C1] | 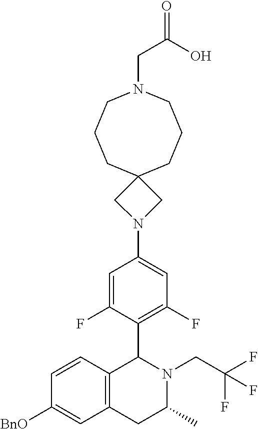 [SMILES: C[C@@H]1Cc2cc(OCc3ccccc3)ccc2C(c2c(F)cc(N3CC4(CCCN(CC(=O)O)CCC4)C3)cc2F)N1CC(F)(F)F] | 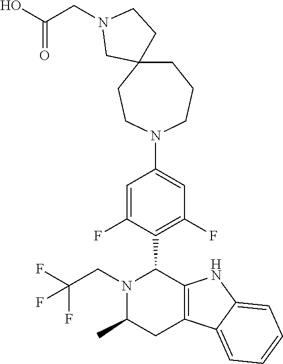 [SMILES: C[C@@H]1Cc2c([nH]c3ccccc23)[C@@H](c2c(F)cc(N3CCCC4(CCN(CC(=O)O)C4)CC3)cc2F)N1CC(F)(F)F] |
| 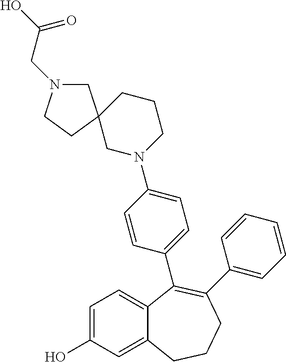 [SMILES: O=C(O)CN1CCC2(CCCN(c3ccc(C4=C(c5ccccc5)CCCc5cc(O)ccc54)cc3)C2)C1] | 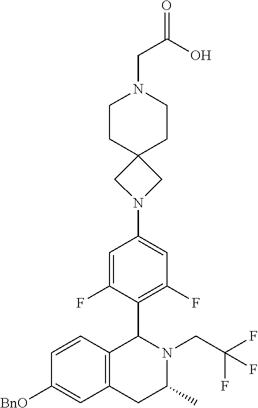 [SMILES: C[C@@H]1Cc2cc(OCc3ccccc3)ccc2C(c2c(F)cc(N3CC4(CCN(CC(=O)O)CC4)C3)cc2F)N1CC(F)(F)F] | 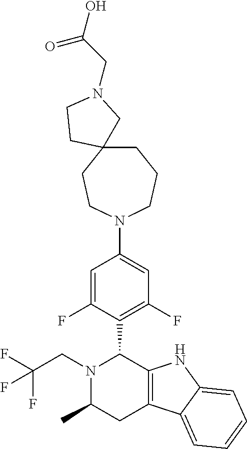 [SMILES: C[C@@H]1Cc2c([nH]c3ccccc23)[C@@H](c2c(F)cc(N3CCCC4(CCN(CC(=O)O)C4)CC3)cc2F)N1CC(F)(F)F] |
| 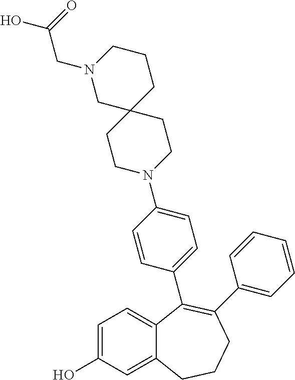 [SMILES: O=C(O)CN1CCCC2(CCN(c3ccc(C4=C(c5ccccc5)CCCc5cc(O)ccc54)cc3)CC2)C1] | 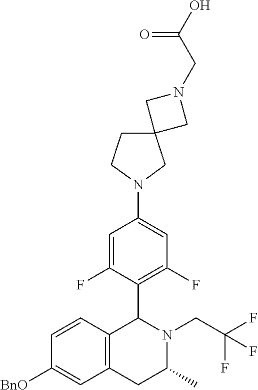 [SMILES: C[C@@H]1Cc2cc(OCc3ccccc3)ccc2C(c2c(F)cc(N3CCC4(CN(CC(=O)O)C4)C3)cc2F)N1CC(F)(F)F] | 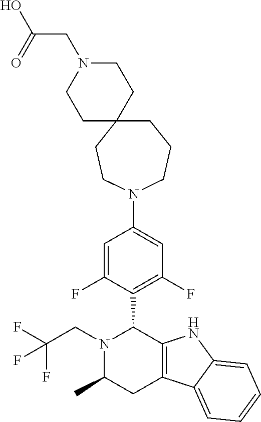 [SMILES: C[C@@H]1Cc2c([nH]c3ccccc23)[C@@H](c2c(F)cc(N3CCCC4(CCN(CC(=O)O)CC4)CC3)cc2F)N1CC(F)(F)F] |
| 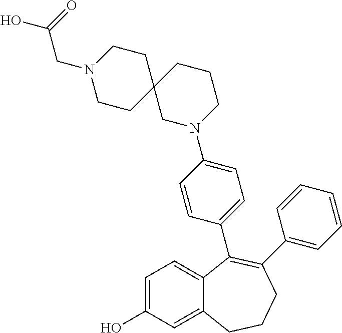 [SMILES: O=C(O)CN1CCC2(CCCN(c3ccc(C4=C(c5ccccc5)CCCc5cc(O)ccc54)cc3)C2)CC1] | 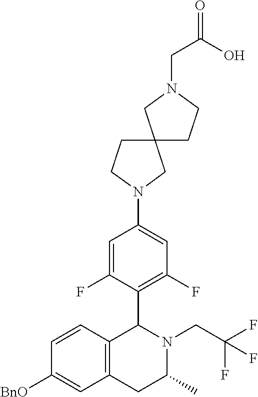 [SMILES: C[C@@H]1Cc2cc(OCc3ccccc3)ccc2C(c2c(F)cc(N3CCC4(CCN(CC(=O)O)C4)C3)cc2F)N1CC(F)(F)F] | 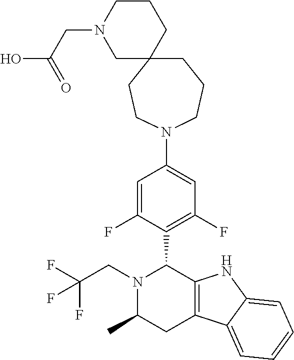 [SMILES: C[C@@H]1Cc2c([nH]c3ccccc23)[C@@H](c2c(F)cc(N3CCCC4(CCCN(CC(=O)O)C4)CC3)cc2F)N1CC(F)(F)F] |
| 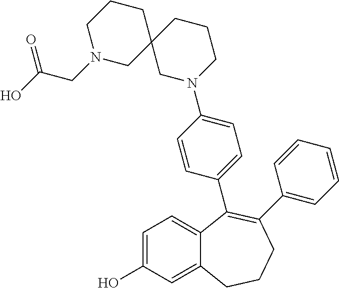 [SMILES: O=C(O)CN1CCCC2(CCCN(c3ccc(C4=C(c5ccccc5)CCCc5cc(O)ccc54)cc3)C2)C1] | 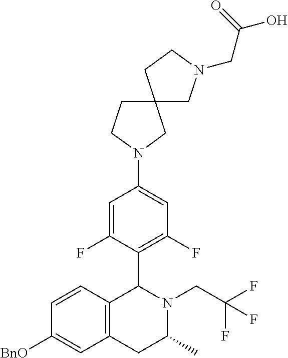 [SMILES: C[C@@H]1Cc2cc(OCc3ccccc3)ccc2C(c2c(F)cc(N3CCC4(CCN(CC(=O)O)C4)C3)cc2F)N1CC(F)(F)F] | 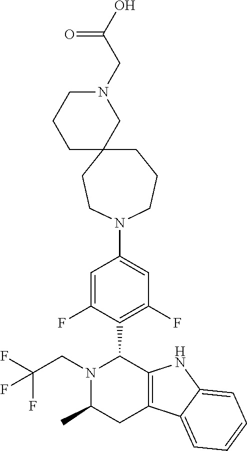 [SMILES: C[C@@H]1Cc2c([nH]c3ccccc23)[C@@H](c2c(F)cc(N3CCCC4(CCCN(CC(=O)O)C4)CC3)cc2F)N1CC(F)(F)F] |
| 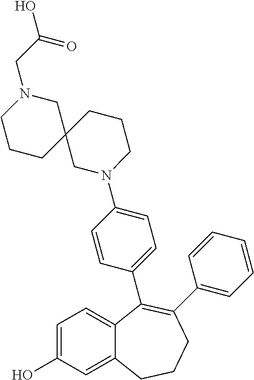 [SMILES: O=C(O)CN1CCCC2(CCCN(c3ccc(C4=C(c5ccccc5)CCCc5cc(O)ccc54)cc3)C2)C1] | 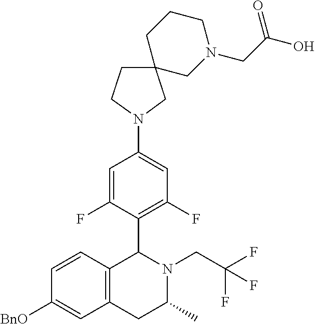 [SMILES: C[C@@H]1Cc2cc(OCc3ccccc3)ccc2C(c2c(F)cc(N3CCC4(CCCN(CC(=O)O)C4)C3)cc2F)N1CC(F)(F)F] | 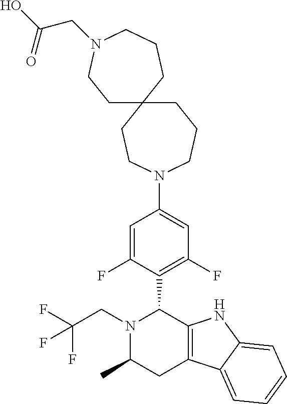 [SMILES: C[C@@H]1Cc2c([nH]c3ccccc23)[C@@H](c2c(F)cc(N3CCCC4(CCCN(CC(=O)O)CC4)CC3)cc2F)N1CC(F)(F)F] |
| 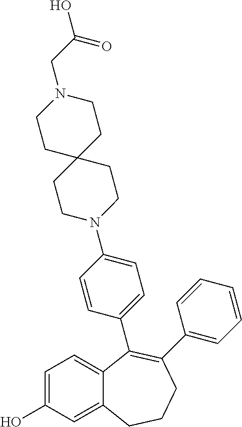 [SMILES: O=C(O)CN1CCC2(CC1)CCN(c1ccc(C3=C(c4ccccc4)CCCc4cc(O)ccc43)cc1)CC2] | 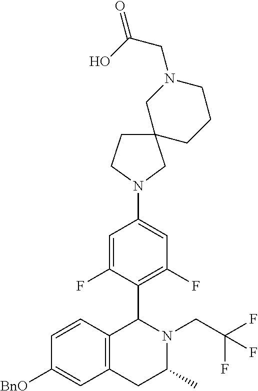 [SMILES: C[C@@H]1Cc2cc(OCc3ccccc3)ccc2C(c2c(F)cc(N3CCC4(CCCN(CC(=O)O)C4)C3)cc2F)N1CC(F)(F)F] | 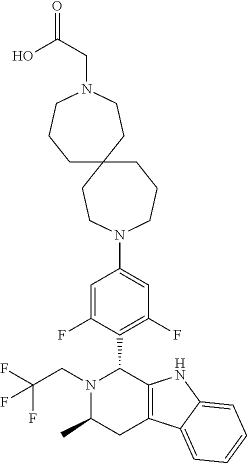 [SMILES: C[C@@H]1Cc2c([nH]c3ccccc23)[C@@H](c2c(F)cc(N3CCCC4(CCCN(CC(=O)O)CC4)CC3)cc2F)N1CC(F)(F)F] |
| 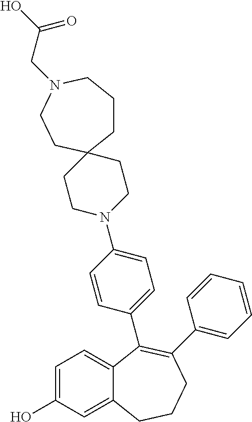 [SMILES: O=C(O)CN1CCCC2(CC1)CCN(c1ccc(C3=C(c4ccccc4)CCCc4cc(O)ccc43)cc1)CC2] | 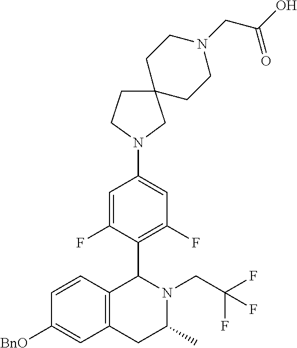 [SMILES: C[C@@H]1Cc2cc(OCc3ccccc3)ccc2C(c2c(F)cc(N3CCC4(CCN(CC(=O)O)CC4)C3)cc2F)N1CC(F)(F)F] | 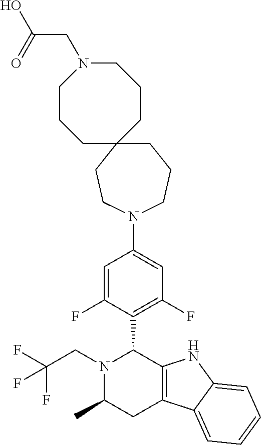 [SMILES: C[C@@H]1Cc2c([nH]c3ccccc23)[C@@H](c2c(F)cc(N3CCCC4(CCCN(CC(=O)O)CCC4)CC3)cc2F)N1CC(F)(F)F] |
| 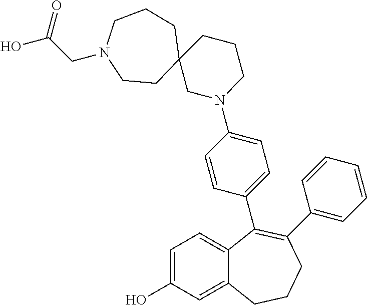 [SMILES: O=C(O)CN1CCCC2(CCCN(c3ccc(C4=C(c5ccccc5)CCCc5cc(O)ccc54)cc3)C2)CC1] | 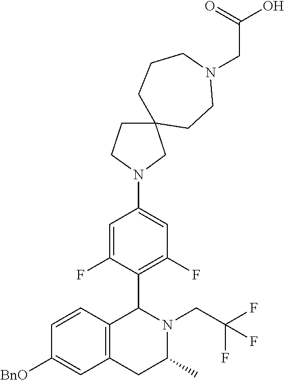 [SMILES: C[C@@H]1Cc2cc(OCc3ccccc3)ccc2C(c2c(F)cc(N3CCC4(CCCN(CC(=O)O)CC4)C3)cc2F)N1CC(F)(F)F] | 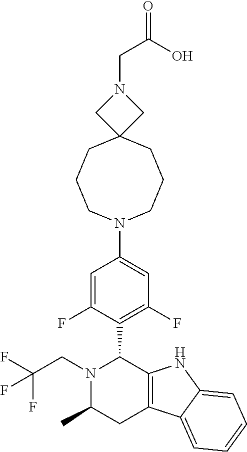 [SMILES: C[C@@H]1Cc2c([nH]c3ccccc23)[C@@H](c2c(F)cc(N3CCCC4(CCC3)CN(CC(=O)O)C4)cc2F)N1CC(F)(F)F] |
| 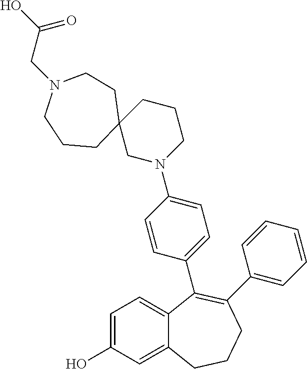 [SMILES: O=C(O)CN1CCCC2(CCCN(c3ccc(C4=C(c5ccccc5)CCCc5cc(O)ccc54)cc3)C2)CC1] | 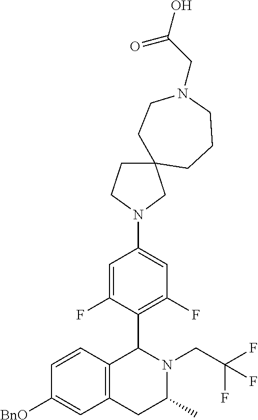 [SMILES: C[C@@H]1Cc2cc(OCc3ccccc3)ccc2C(c2c(F)cc(N3CCC4(CCCN(CC(=O)O)CC4)C3)cc2F)N1CC(F)(F)F] | 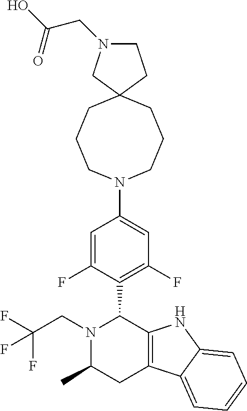 [SMILES: C[C@@H]1Cc2c([nH]c3ccccc23)[C@@H](c2c(F)cc(N3CCCC4(CCC3)CCN(CC(=O)O)C4)cc2F)N1CC(F)(F)F] |
| 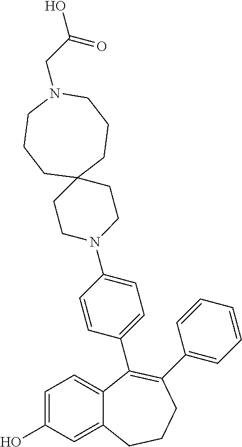 [SMILES: O=C(O)CN1CCCC2(CCC1)CCN(c1ccc(C3=C(c4ccccc4)CCCc4cc(O)ccc43)cc1)CC2] | 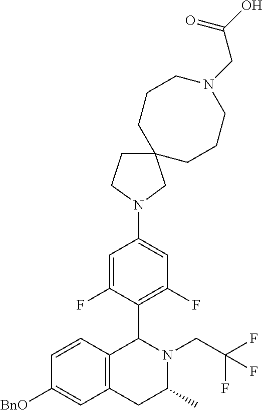 [SMILES: C[C@@H]1Cc2cc(OCc3ccccc3)ccc2C(c2c(F)cc(N3CCC4(CCCN(CC(=O)O)CCC4)C3)cc2F)N1CC(F)(F)F] | 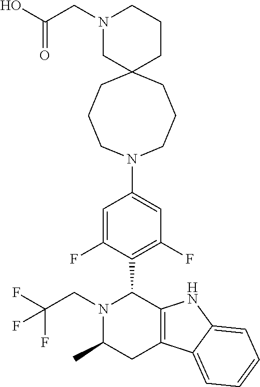 [SMILES: C[C@@H]1Cc2c([nH]c3ccccc23)[C@@H](c2c(F)cc(N3CCCC4(CCCN(CC(=O)O)C4)CCC3)cc2F)N1CC(F)(F)F] |
| 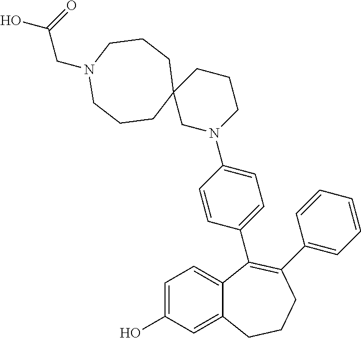 [SMILES: O=C(O)CN1CCCC2(CCC1)CCCN(c1ccc(C3=C(c4ccccc4)CCCc4cc(O)ccc43)cc1)C2] | 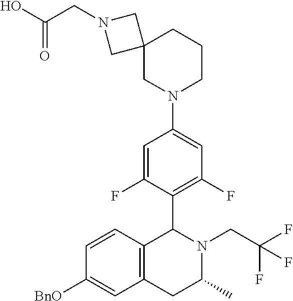 [SMILES: C[C@@H]1Cc2cc(OCc3ccccc3)ccc2C(c2c(F)cc(N3CCCC4(CN(CC(=O)O)C4)C3)cc2F)N1CC(F)(F)F] | 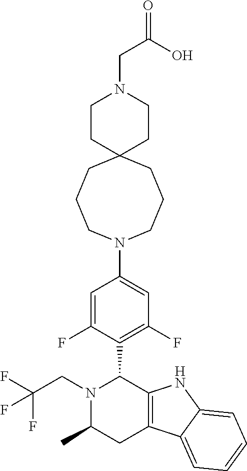 [SMILES: C[C@@H]1Cc2c([nH]c3ccccc23)[C@@H](c2c(F)cc(N3CCCC4(CCC3)CCN(CC(=O)O)CC4)cc2F)N1CC(F)(F)F] |
| 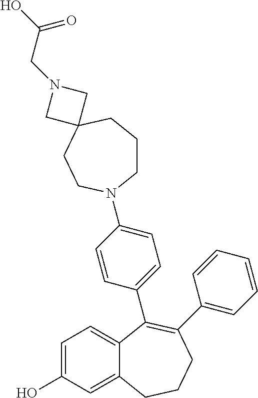 [SMILES: O=C(O)CN1CC2(CCCN(c3ccc(C4=C(c5ccccc5)CCCc5cc(O)ccc54)cc3)CC2)C1] | 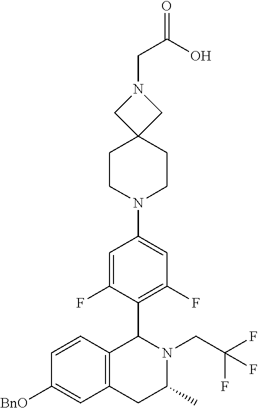 [SMILES: C[C@@H]1Cc2cc(OCc3ccccc3)ccc2C(c2c(F)cc(N3CCC4(CC3)CN(CC(=O)O)C4)cc2F)N1CC(F)(F)F] | 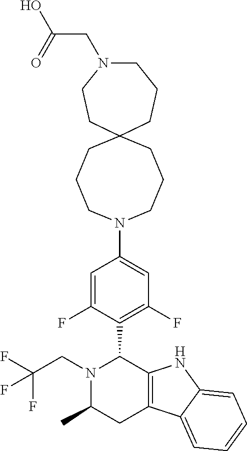 [SMILES: C[C@@H]1Cc2c([nH]c3ccccc23)[C@@H](c2c(F)cc(N3CCCC4(CCCN(CC(=O)O)CC4)CCC3)cc2F)N1CC(F)(F)F] |
| 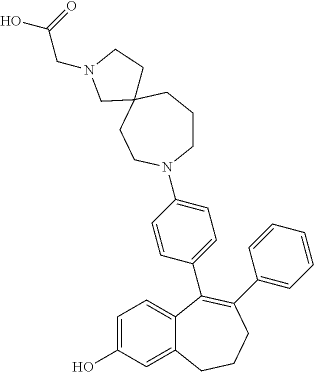 [SMILES: O=C(O)CN1CCC2(CCCN(c3ccc(C4=C(c5ccccc5)CCCc5cc(O)ccc54)cc3)CC2)C1] | 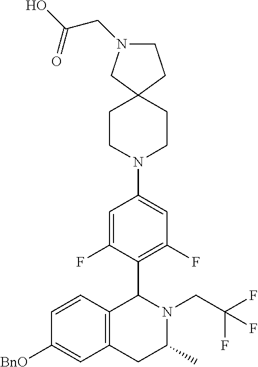 [SMILES: C[C@@H]1Cc2cc(OCc3ccccc3)ccc2C(c2c(F)cc(N3CCC4(CCN(CC(=O)O)C4)CC3)cc2F)N1CC(F)(F)F] | 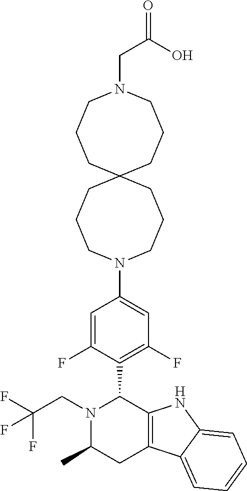 [SMILES: C[C@@H]1Cc2c([nH]c3ccccc23)[C@@H](c2c(F)cc(N3CCCC4(CCCN(CC(=O)O)CCC4)CCC3)cc2F)N1CC(F)(F)F] |
| 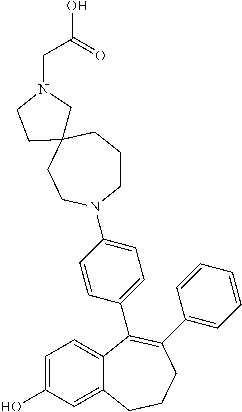 [SMILES: O=C(O)CN1CCC2(CCCN(c3ccc(C4=C(c5ccccc5)CCCc5cc(O)ccc54)cc3)CC2)C1] | 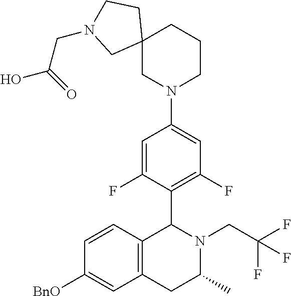 [SMILES: C[C@@H]1Cc2cc(OCc3ccccc3)ccc2C(c2c(F)cc(N3CCCC4(CCN(CC(=O)O)C4)C3)cc2F)N1CC(F)(F)F] | 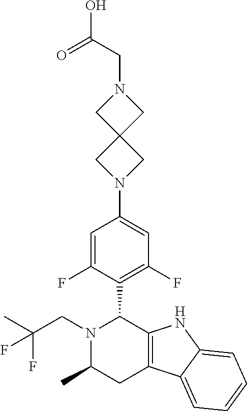 [SMILES: C[C@@H]1Cc2c([nH]c3ccccc23)[C@@H](c2c(F)cc(N3CC4(CN(CC(=O)O)C4)C3)cc2F)N1CC(C)(F)F] |
| 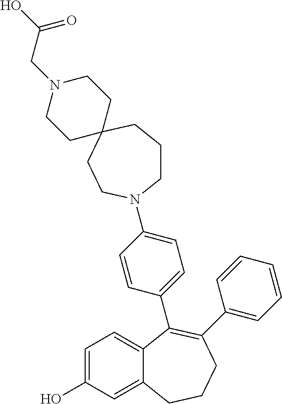 [SMILES: O=C(O)CN1CCC2(CCCN(c3ccc(C4=C(c5ccccc5)CCCc5cc(O)ccc54)cc3)CC2)CC1] | 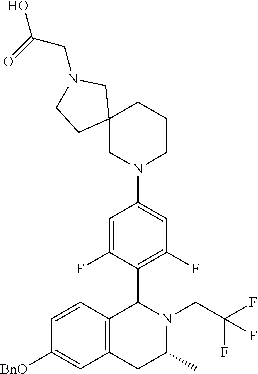 [SMILES: C[C@@H]1Cc2cc(OCc3ccccc3)ccc2C(c2c(F)cc(N3CCCC4(CCN(CC(=O)O)C4)C3)cc2F)N1CC(F)(F)F] | 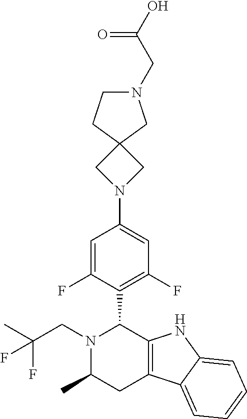 [SMILES: C[C@@H]1Cc2c([nH]c3ccccc23)[C@@H](c2c(F)cc(N3CC4(CCN(CC(=O)O)C4)C3)cc2F)N1CC(C)(F)F] |
| 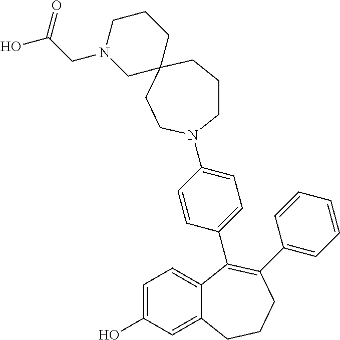 [SMILES: O=C(O)CN1CCCC2(CCCN(c3ccc(C4=C(c5ccccc5)CCCc5cc(O)ccc54)cc3)CC2)C1] | 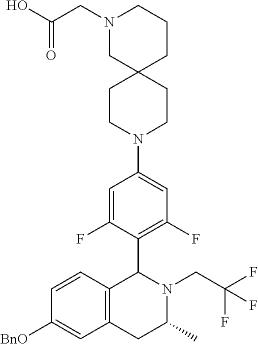 [SMILES: C[C@@H]1Cc2cc(OCc3ccccc3)ccc2C(c2c(F)cc(N3CCC4(CCCN(CC(=O)O)C4)CC3)cc2F)N1CC(F)(F)F] | 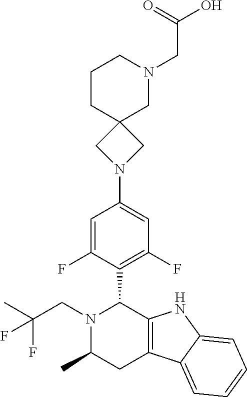 [SMILES: C[C@@H]1Cc2c([nH]c3ccccc23)[C@@H](c2c(F)cc(N3CC4(CCCN(CC(=O)O)C4)C3)cc2F)N1CC(C)(F)F] |
| 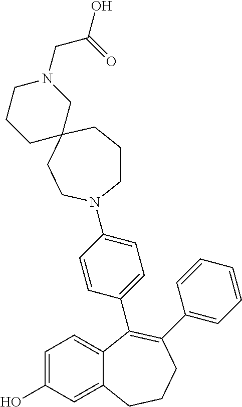 [SMILES: O=C(O)CN1CCCC2(CCCN(c3ccc(C4=C(c5ccccc5)CCCc5cc(O)ccc54)cc3)CC2)C1] | 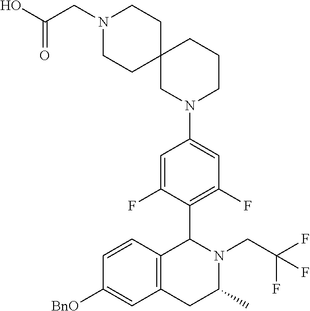 [SMILES: C[C@@H]1Cc2cc(OCc3ccccc3)ccc2C(c2c(F)cc(N3CCCC4(CCN(CC(=O)O)CC4)C3)cc2F)N1CC(F)(F)F] | 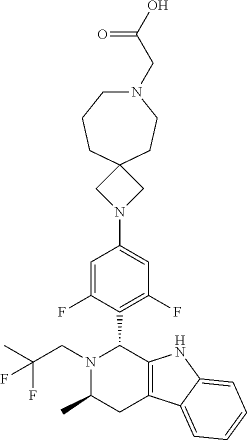 [SMILES: C[C@@H]1Cc2c([nH]c3ccccc23)[C@@H](c2c(F)cc(N3CC4(CCCN(CC(=O)O)CC4)C3)cc2F)N1CC(C)(F)F] |
| 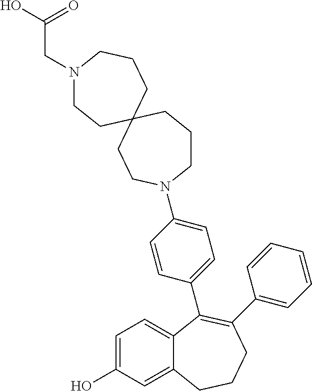 [SMILES: O=C(O)CN1CCCC2(CCCN(c3ccc(C4=C(c5ccccc5)CCCc5cc(O)ccc54)cc3)CC2)CC1] | 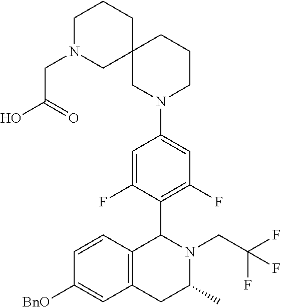 [SMILES: C[C@@H]1Cc2cc(OCc3ccccc3)ccc2C(c2c(F)cc(N3CCCC4(CCCN(CC(=O)O)C4)C3)cc2F)N1CC(F)(F)F] | 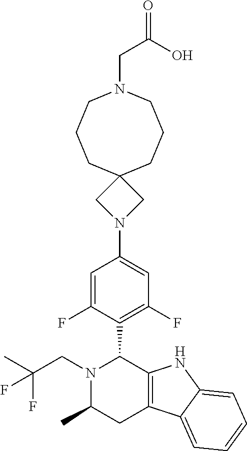 [SMILES: C[C@@H]1Cc2c([nH]c3ccccc23)[C@@H](c2c(F)cc(N3CC4(CCCN(CC(=O)O)CCC4)C3)cc2F)N1CC(C)(F)F] |
| 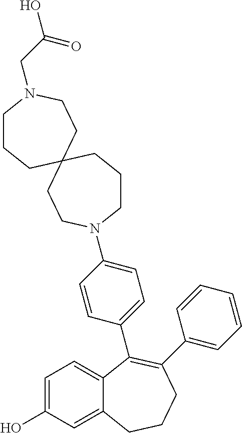 [SMILES: O=C(O)CN1CCCC2(CCCN(c3ccc(C4=C(c5ccccc5)CCCc5cc(O)ccc54)cc3)CC2)CC1] | 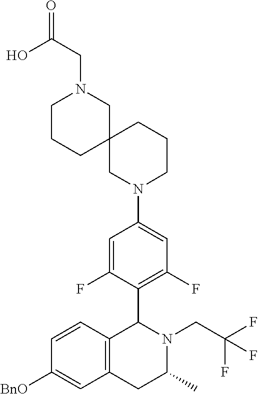 [SMILES: C[C@@H]1Cc2cc(OCc3ccccc3)ccc2C(c2c(F)cc(N3CCCC4(CCCN(CC(=O)O)C4)C3)cc2F)N1CC(F)(F)F] | 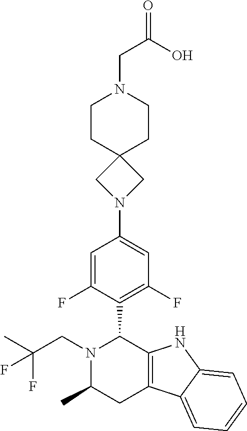 [SMILES: C[C@@H]1Cc2c([nH]c3ccccc23)[C@@H](c2c(F)cc(N3CC4(CCN(CC(=O)O)CC4)C3)cc2F)N1CC(C)(F)F] |
| 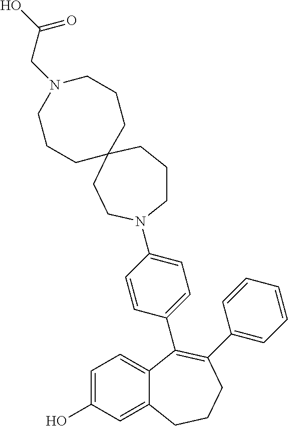 [SMILES: O=C(O)CN1CCCC2(CCC1)CCCN(c1ccc(C3=C(c4ccccc4)CCCc4cc(O)ccc43)cc1)CC2] | 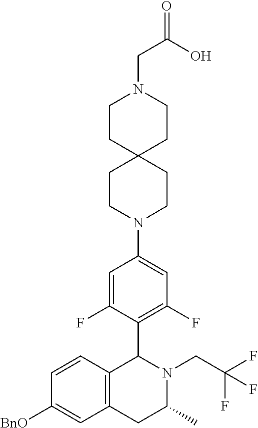 [SMILES: C[C@@H]1Cc2cc(OCc3ccccc3)ccc2C(c2c(F)cc(N3CCC4(CCN(CC(=O)O)CC4)CC3)cc2F)N1CC(F)(F)F] | 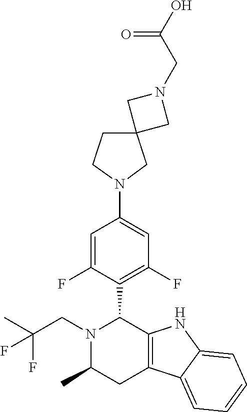 [SMILES: C[C@@H]1Cc2c([nH]c3ccccc23)[C@@H](c2c(F)cc(N3CCC4(CN(CC(=O)O)C4)C3)cc2F)N1CC(C)(F)F] |
| 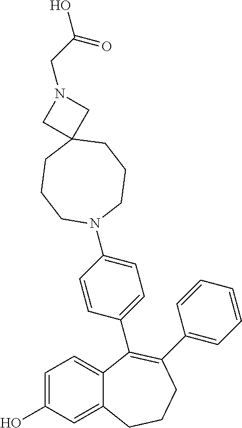 [SMILES: O=C(O)CN1CC2(CCCN(c3ccc(C4=C(c5ccccc5)CCCc5cc(O)ccc54)cc3)CCC2)C1] | 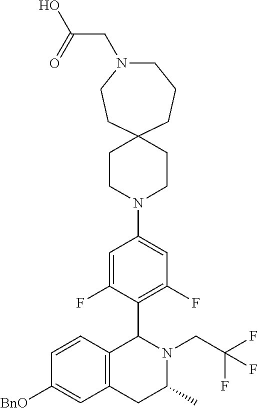 [SMILES: C[C@@H]1Cc2cc(OCc3ccccc3)ccc2C(c2c(F)cc(N3CCC4(CCCN(CC(=O)O)CC4)CC3)cc2F)N1CC(F)(F)F] | 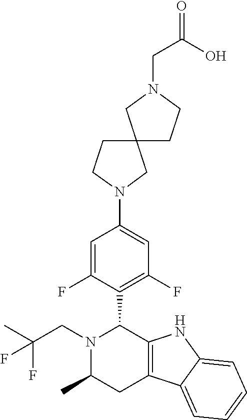 [SMILES: C[C@@H]1Cc2c([nH]c3ccccc23)[C@@H](c2c(F)cc(N3CCC4(CCN(CC(=O)O)C4)C3)cc2F)N1CC(C)(F)F] |
| 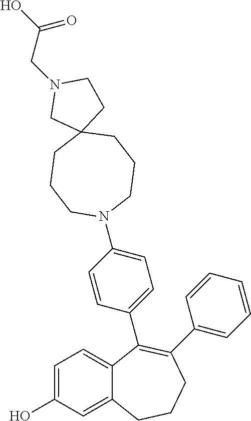 [SMILES: O=C(O)CN1CCC2(CCCN(c3ccc(C4=C(c5ccccc5)CCCc5cc(O)ccc54)cc3)CCC2)C1] | 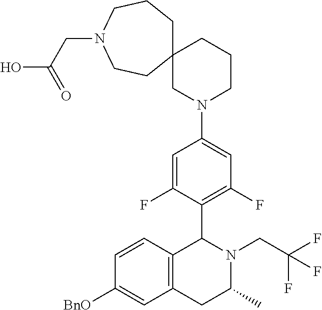 [SMILES: C[C@@H]1Cc2cc(OCc3ccccc3)ccc2C(c2c(F)cc(N3CCCC4(CCCN(CC(=O)O)CC4)C3)cc2F)N1CC(F)(F)F] | 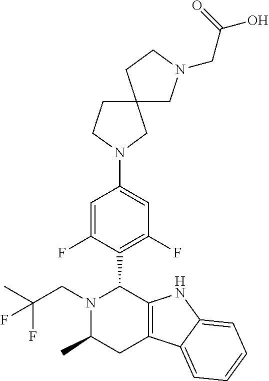 [SMILES: C[C@@H]1Cc2c([nH]c3ccccc23)[C@@H](c2c(F)cc(N3CCC4(CCN(CC(=O)O)C4)C3)cc2F)N1CC(C)(F)F] |
| 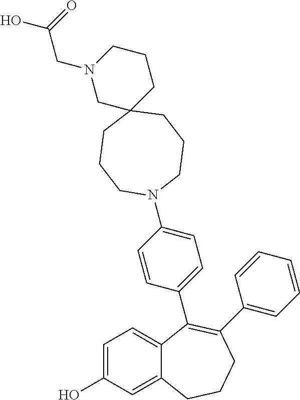 [SMILES: O=C(O)CN1CCCC2(CCCN(c3ccc(C4=C(c5ccccc5)CCCc5cc(O)ccc54)cc3)CCC2)C1] | 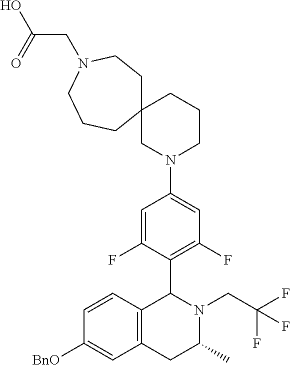 [SMILES: C[C@@H]1Cc2cc(OCc3ccccc3)ccc2C(c2c(F)cc(N3CCCC4(CCCN(CC(=O)O)CC4)C3)cc2F)N1CC(F)(F)F] | 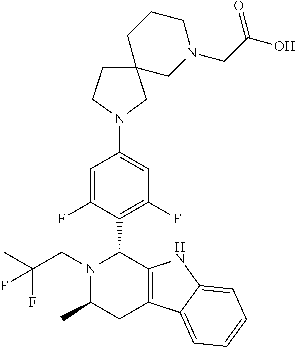 [SMILES: C[C@@H]1Cc2c([nH]c3ccccc23)[C@@H](c2c(F)cc(N3CCC4(CCCN(CC(=O)O)C4)C3)cc2F)N1CC(C)(F)F] |
| 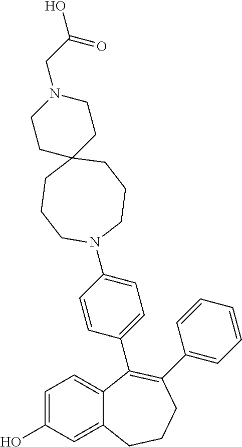 [SMILES: O=C(O)CN1CCC2(CCCN(c3ccc(C4=C(c5ccccc5)CCCc5cc(O)ccc54)cc3)CCC2)CC1] | 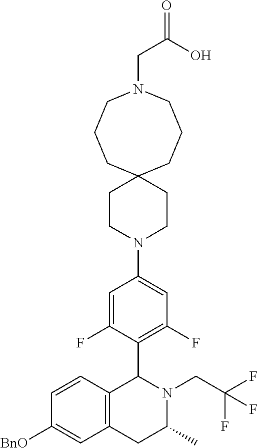 [SMILES: C[C@@H]1Cc2cc(OCc3ccccc3)ccc2C(c2c(F)cc(N3CCC4(CCCN(CC(=O)O)CCC4)CC3)cc2F)N1CC(F)(F)F] | 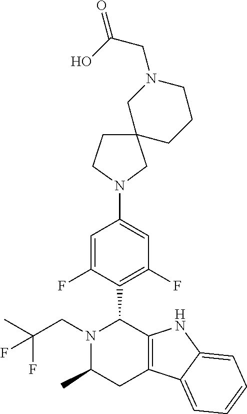 [SMILES: C[C@@H]1Cc2c([nH]c3ccccc23)[C@@H](c2c(F)cc(N3CCC4(CCCN(CC(=O)O)C4)C3)cc2F)N1CC(C)(F)F] |
| 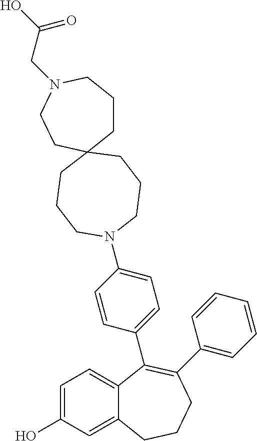 [SMILES: O=C(O)CN1CCCC2(CCCN(c3ccc(C4=C(c5ccccc5)CCCc5cc(O)ccc54)cc3)CCC2)CC1] | 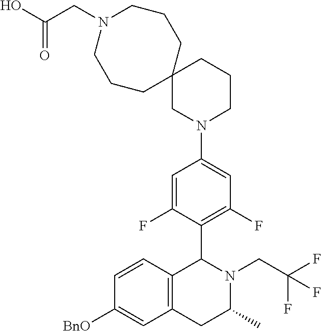 [SMILES: C[C@@H]1Cc2cc(OCc3ccccc3)ccc2C(c2c(F)cc(N3CCCC4(CCCN(CC(=O)O)CCC4)C3)cc2F)N1CC(F)(F)F] | 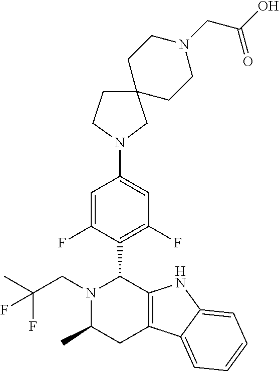 [SMILES: C[C@@H]1Cc2c([nH]c3ccccc23)[C@@H](c2c(F)cc(N3CCC4(CCN(CC(=O)O)CC4)C3)cc2F)N1CC(C)(F)F] |
| 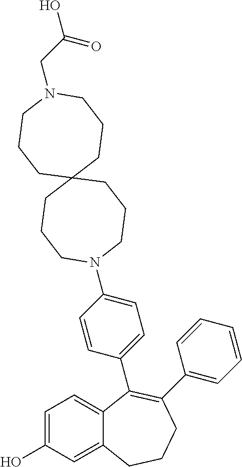 [SMILES: O=C(O)CN1CCCC2(CCC1)CCCN(c1ccc(C3=C(c4ccccc4)CCCc4cc(O)ccc43)cc1)CCC2] | 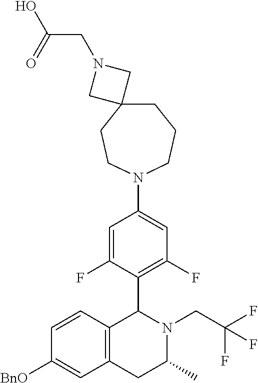 [SMILES: C[C@@H]1Cc2cc(OCc3ccccc3)ccc2C(c2c(F)cc(N3CCCC4(CC3)CN(CC(=O)O)C4)cc2F)N1CC(F)(F)F] | 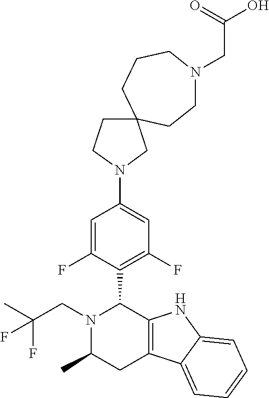 [SMILES: C[C@@H]1Cc2c([nH]c3ccccc23)[C@@H](c2c(F)cc(N3CCC4(CCCN(CC(=O)O)CC4)C3)cc2F)N1CC(C)(F)F] |
| 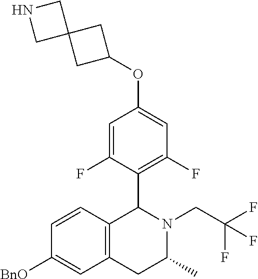 [SMILES: C[C@@H]1Cc2cc(OCc3ccccc3)ccc2C(c2c(F)cc(OC3CC4(CNC4)C3)cc2F)N1CC(F)(F)F] | 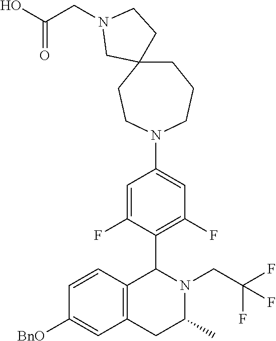 [SMILES: C[C@@H]1Cc2cc(OCc3ccccc3)ccc2C(c2c(F)cc(N3CCCC4(CCN(CC(=O)O)C4)CC3)cc2F)N1CC(F)(F)F] | 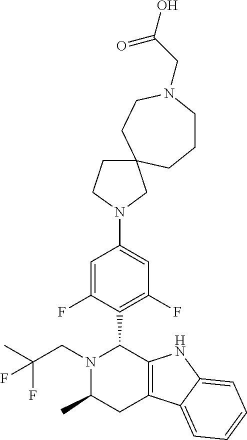 [SMILES: C[C@@H]1Cc2c([nH]c3ccccc23)[C@@H](c2c(F)cc(N3CCC4(CCCN(CC(=O)O)CC4)C3)cc2F)N1CC(C)(F)F] |
| 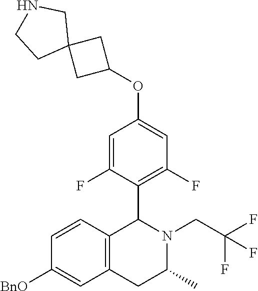 [SMILES: C[C@@H]1Cc2cc(OCc3ccccc3)ccc2C(c2c(F)cc(OC3CC4(CCNC4)C3)cc2F)N1CC(F)(F)F] | 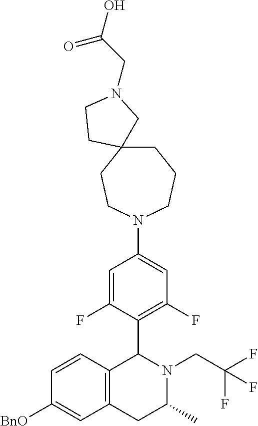 [SMILES: C[C@@H]1Cc2cc(OCc3ccccc3)ccc2C(c2c(F)cc(N3CCCC4(CCN(CC(=O)O)C4)CC3)cc2F)N1CC(F)(F)F] | 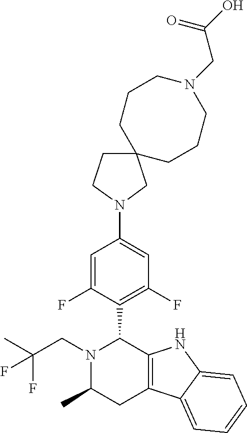 [SMILES: C[C@@H]1Cc2c([nH]c3ccccc23)[C@@H](c2c(F)cc(N3CCC4(CCCN(CC(=O)O)CCC4)C3)cc2F)N1CC(C)(F)F] |
| 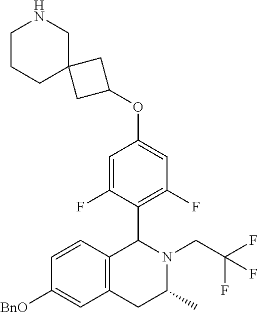 [SMILES: C[C@@H]1Cc2cc(OCc3ccccc3)ccc2C(c2c(F)cc(OC3CC4(CCCNC4)C3)cc2F)N1CC(F)(F)F] | 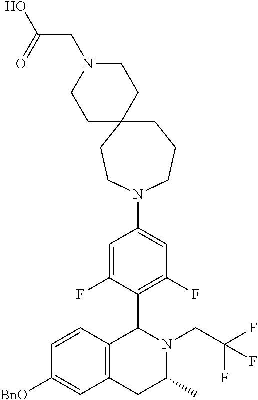 [SMILES: C[C@@H]1Cc2cc(OCc3ccccc3)ccc2C(c2c(F)cc(N3CCCC4(CCN(CC(=O)O)CC4)CC3)cc2F)N1CC(F)(F)F] | 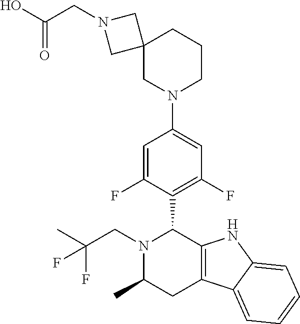 [SMILES: C[C@@H]1Cc2c([nH]c3ccccc23)[C@@H](c2c(F)cc(N3CCCC4(CN(CC(=O)O)C4)C3)cc2F)N1CC(C)(F)F] |
| 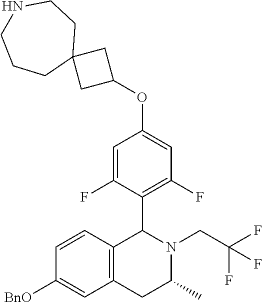 [SMILES: C[C@@H]1Cc2cc(OCc3ccccc3)ccc2C(c2c(F)cc(OC3CC4(CCCNCC4)C3)cc2F)N1CC(F)(F)F] | 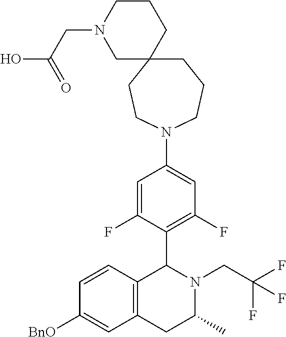 [SMILES: C[C@@H]1Cc2cc(OCc3ccccc3)ccc2C(c2c(F)cc(N3CCCC4(CCCN(CC(=O)O)C4)CC3)cc2F)N1CC(F)(F)F] | 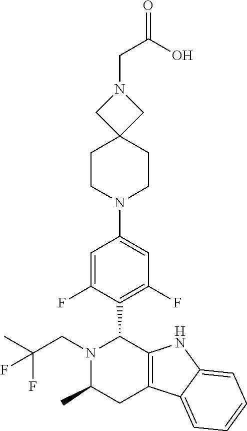 [SMILES: C[C@@H]1Cc2c([nH]c3ccccc23)[C@@H](c2c(F)cc(N3CCC4(CC3)CN(CC(=O)O)C4)cc2F)N1CC(C)(F)F] |
| 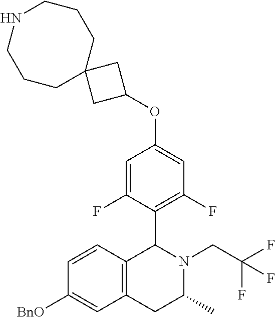 [SMILES: C[C@@H]1Cc2cc(OCc3ccccc3)ccc2C(c2c(F)cc(OC3CC4(CCCNCCC4)C3)cc2F)N1CC(F)(F)F] | 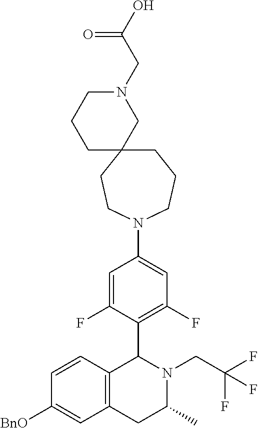 [SMILES: C[C@@H]1Cc2cc(OCc3ccccc3)ccc2C(c2c(F)cc(N3CCCC4(CCCN(CC(=O)O)C4)CC3)cc2F)N1CC(F)(F)F] | 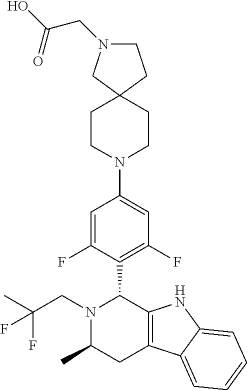 [SMILES: C[C@@H]1Cc2c([nH]c3ccccc23)[C@@H](c2c(F)cc(N3CCC4(CCN(CC(=O)O)C4)CC3)cc2F)N1CC(C)(F)F] |
| 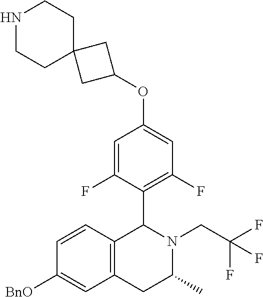 [SMILES: C[C@@H]1Cc2cc(OCc3ccccc3)ccc2C(c2c(F)cc(OC3CC4(CCNCC4)C3)cc2F)N1CC(F)(F)F] | 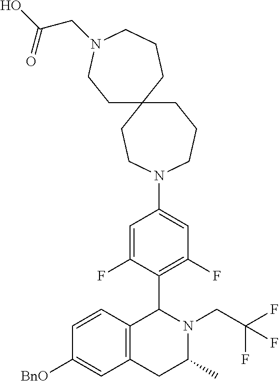 [SMILES: C[C@@H]1Cc2cc(OCc3ccccc3)ccc2C(c2c(F)cc(N3CCCC4(CCCN(CC(=O)O)CC4)CC3)cc2F)N1CC(F)(F)F] | 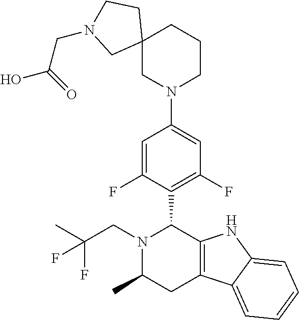 [SMILES: C[C@@H]1Cc2c([nH]c3ccccc23)[C@@H](c2c(F)cc(N3CCCC4(CCN(CC(=O)O)C4)C3)cc2F)N1CC(C)(F)F] |
| 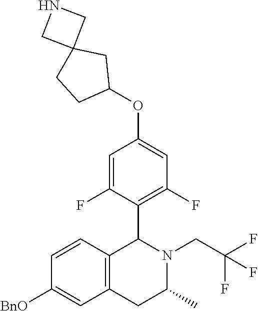 [SMILES: C[C@@H]1Cc2cc(OCc3ccccc3)ccc2C(c2c(F)cc(OC3CCC4(CNC4)C3)cc2F)N1CC(F)(F)F] | 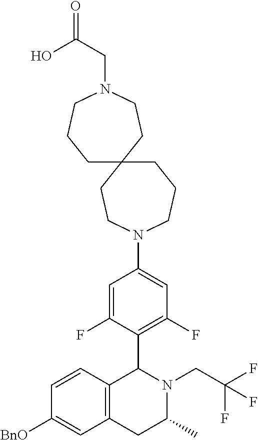 [SMILES: C[C@@H]1Cc2cc(OCc3ccccc3)ccc2C(c2c(F)cc(N3CCCC4(CCCN(CC(=O)O)CC4)CC3)cc2F)N1CC(F)(F)F] | 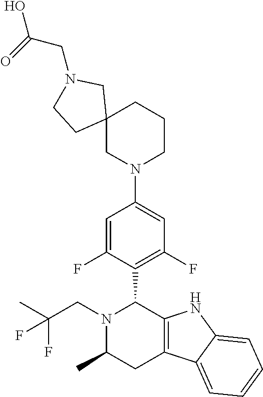 [SMILES: C[C@@H]1Cc2c([nH]c3ccccc23)[C@@H](c2c(F)cc(N3CCCC4(CCN(CC(=O)O)C4)C3)cc2F)N1CC(C)(F)F] |
| 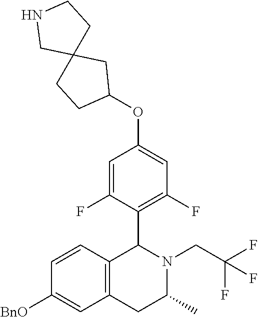 [SMILES: C[C@@H]1Cc2cc(OCc3ccccc3)ccc2C(c2c(F)cc(OC3CCC4(CCNC4)C3)cc2F)N1CC(F)(F)F] | 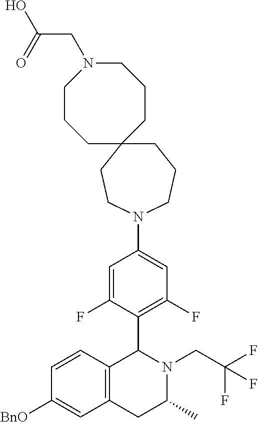 [SMILES: C[C@@H]1Cc2cc(OCc3ccccc3)ccc2C(c2c(F)cc(N3CCCC4(CCCN(CC(=O)O)CCC4)CC3)cc2F)N1CC(F)(F)F] | 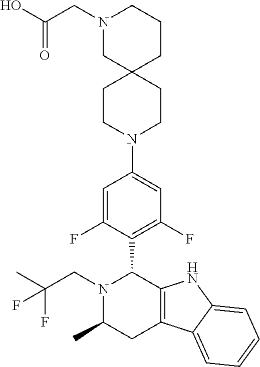 [SMILES: C[C@@H]1Cc2c([nH]c3ccccc23)[C@@H](c2c(F)cc(N3CCC4(CCCN(CC(=O)O)C4)CC3)cc2F)N1CC(C)(F)F] |
| 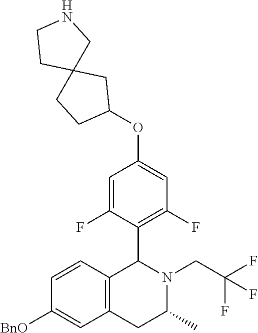 [SMILES: C[C@@H]1Cc2cc(OCc3ccccc3)ccc2C(c2c(F)cc(OC3CCC4(CCNC4)C3)cc2F)N1CC(F)(F)F] | 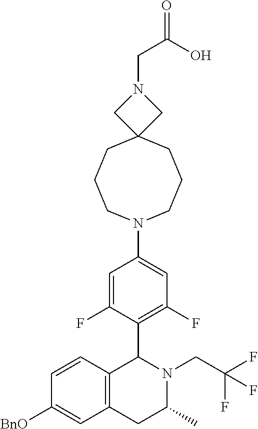 [SMILES: C[C@@H]1Cc2cc(OCc3ccccc3)ccc2C(c2c(F)cc(N3CCCC4(CCC3)CN(CC(=O)O)C4)cc2F)N1CC(F)(F)F] | 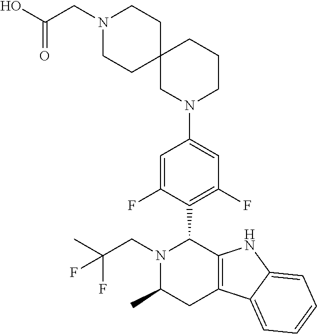 [SMILES: C[C@@H]1Cc2c([nH]c3ccccc23)[C@@H](c2c(F)cc(N3CCCC4(CCN(CC(=O)O)CC4)C3)cc2F)N1CC(C)(F)F] |
| 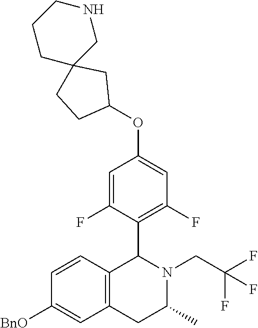 [SMILES: C[C@@H]1Cc2cc(OCc3ccccc3)ccc2C(c2c(F)cc(OC3CCC4(CCCNC4)C3)cc2F)N1CC(F)(F)F] | 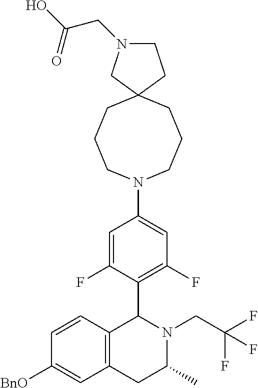 [SMILES: C[C@@H]1Cc2cc(OCc3ccccc3)ccc2C(c2c(F)cc(N3CCCC4(CCC3)CCN(CC(=O)O)C4)cc2F)N1CC(F)(F)F] | 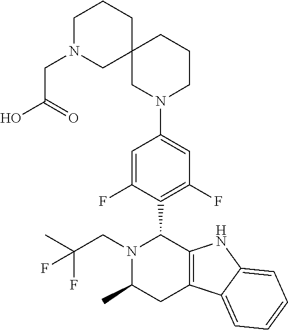 [SMILES: C[C@@H]1Cc2c([nH]c3ccccc23)[C@@H](c2c(F)cc(N3CCCC4(CCCN(CC(=O)O)C4)C3)cc2F)N1CC(C)(F)F] |
| 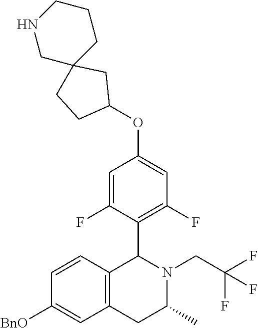 [SMILES: C[C@@H]1Cc2cc(OCc3ccccc3)ccc2C(c2c(F)cc(OC3CCC4(CCCNC4)C3)cc2F)N1CC(F)(F)F] | 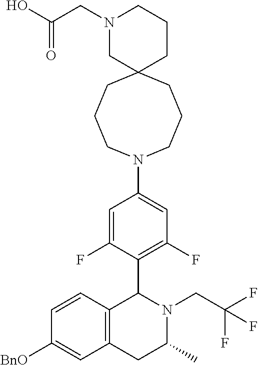 [SMILES: C[C@@H]1Cc2cc(OCc3ccccc3)ccc2C(c2c(F)cc(N3CCCC4(CCCN(CC(=O)O)C4)CCC3)cc2F)N1CC(F)(F)F] | 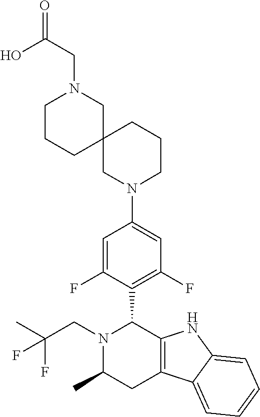 [SMILES: C[C@@H]1Cc2c([nH]c3ccccc23)[C@@H](c2c(F)cc(N3CCCC4(CCCN(CC(=O)O)C4)C3)cc2F)N1CC(C)(F)F] |
| 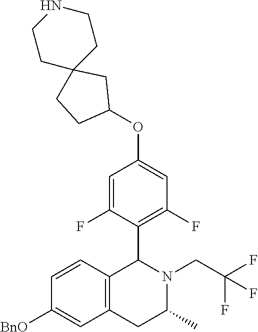 [SMILES: C[C@@H]1Cc2cc(OCc3ccccc3)ccc2C(c2c(F)cc(OC3CCC4(CCNCC4)C3)cc2F)N1CC(F)(F)F] | 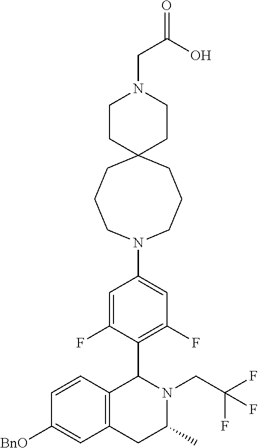 [SMILES: C[C@@H]1Cc2cc(OCc3ccccc3)ccc2C(c2c(F)cc(N3CCCC4(CCC3)CCN(CC(=O)O)CC4)cc2F)N1CC(F)(F)F] | 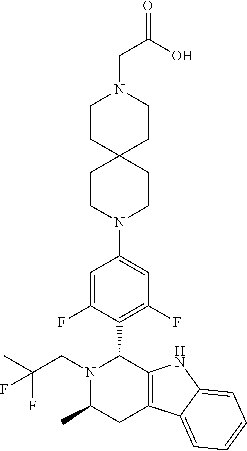 [SMILES: C[C@@H]1Cc2c([nH]c3ccccc23)[C@@H](c2c(F)cc(N3CCC4(CCN(CC(=O)O)CC4)CC3)cc2F)N1CC(C)(F)F] |
| 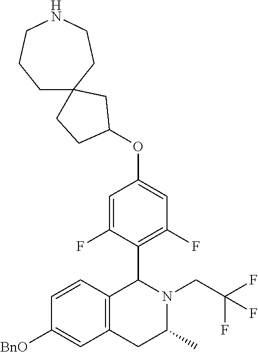 [SMILES: C[C@@H]1Cc2cc(OCc3ccccc3)ccc2C(c2c(F)cc(OC3CCC4(CCCNCC4)C3)cc2F)N1CC(F)(F)F] | 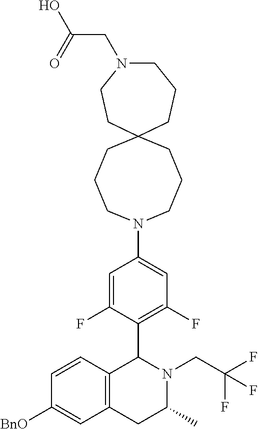 [SMILES: C[C@@H]1Cc2cc(OCc3ccccc3)ccc2C(c2c(F)cc(N3CCCC4(CCCN(CC(=O)O)CC4)CCC3)cc2F)N1CC(F)(F)F] | 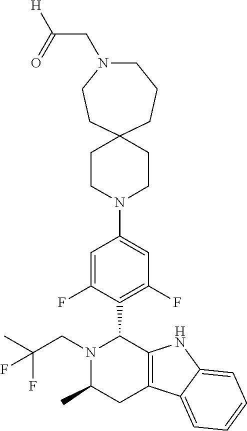 [SMILES: C[C@@H]1Cc2c([nH]c3ccccc23)[C@@H](c2c(F)cc(N3CCC4(CCCN(CC=O)CC4)CC3)cc2F)N1CC(C)(F)F] |
| 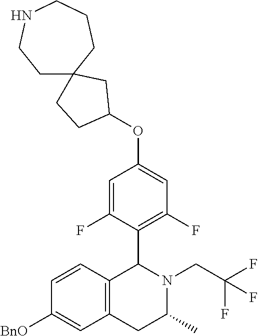 [SMILES: C[C@@H]1Cc2cc(OCc3ccccc3)ccc2C(c2c(F)cc(OC3CCC4(CCCNCC4)C3)cc2F)N1CC(F)(F)F] | 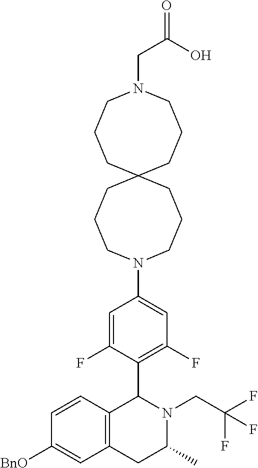 [SMILES: C[C@@H]1Cc2cc(OCc3ccccc3)ccc2C(c2c(F)cc(N3CCCC4(CCCN(CC(=O)O)CCC4)CCC3)cc2F)N1CC(F)(F)F] | 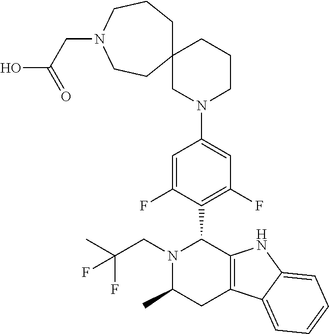 [SMILES: C[C@@H]1Cc2c([nH]c3ccccc23)[C@@H](c2c(F)cc(N3CCCC4(CCCN(CC(=O)O)CC4)C3)cc2F)N1CC(C)(F)F] |
| 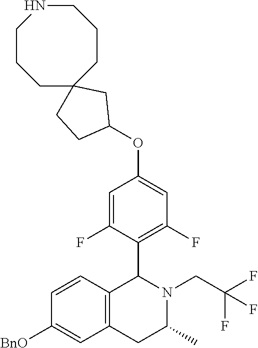 [SMILES: C[C@@H]1Cc2cc(OCc3ccccc3)ccc2C(c2c(F)cc(OC3CCC4(CCCNCCC4)C3)cc2F)N1CC(F)(F)F] | 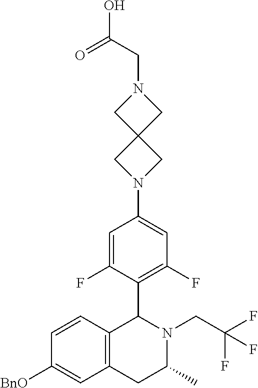 [SMILES: C[C@@H]1Cc2cc(OCc3ccccc3)ccc2C(c2c(F)cc(N3CC4(CN(CC(=O)O)C4)C3)cc2F)N1CC(F)(F)F] | 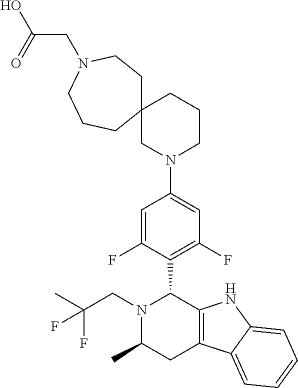 [SMILES: C[C@@H]1Cc2c([nH]c3ccccc23)[C@@H](c2c(F)cc(N3CCCC4(CCCN(CC(=O)O)CC4)C3)cc2F)N1CC(C)(F)F] |
| 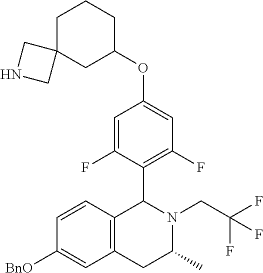 [SMILES: C[C@@H]1Cc2cc(OCc3ccccc3)ccc2C(c2c(F)cc(OC3CCCC4(CNC4)C3)cc2F)N1CC(F)(F)F] | 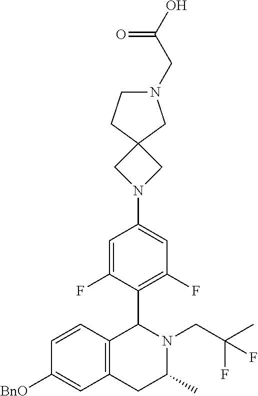 [SMILES: C[C@@H]1Cc2cc(OCc3ccccc3)ccc2C(c2c(F)cc(N3CC4(CCN(CC(=O)O)C4)C3)cc2F)N1CC(C)(F)F] | 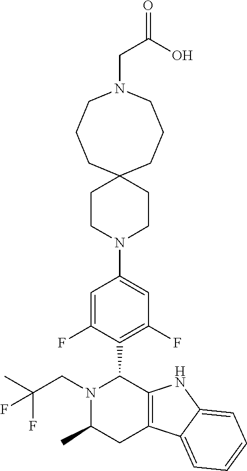 [SMILES: C[C@@H]1Cc2c([nH]c3ccccc23)[C@@H](c2c(F)cc(N3CCC4(CCCN(CC(=O)O)CCC4)CC3)cc2F)N1CC(C)(F)F] |
| 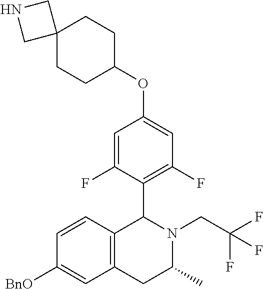 [SMILES: C[C@@H]1Cc2cc(OCc3ccccc3)ccc2C(c2c(F)cc(OC3CCC4(CC3)CNC4)cc2F)N1CC(F)(F)F] | 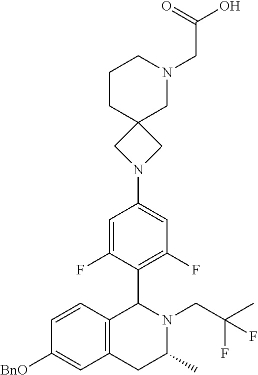 [SMILES: C[C@@H]1Cc2cc(OCc3ccccc3)ccc2C(c2c(F)cc(N3CC4(CCCN(CC(=O)O)C4)C3)cc2F)N1CC(C)(F)F] | 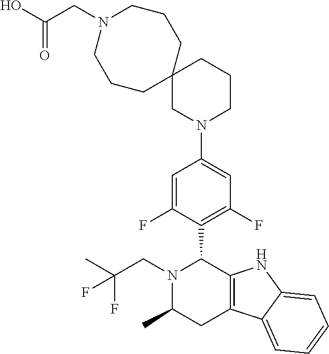 [SMILES: C[C@@H]1Cc2c([nH]c3ccccc23)[C@@H](c2c(F)cc(N3CCCC4(CCCN(CC(=O)O)CCC4)C3)cc2F)N1CC(C)(F)F] |
| 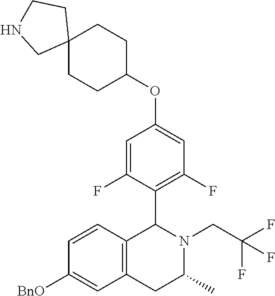 [SMILES: C[C@@H]1Cc2cc(OCc3ccccc3)ccc2C(c2c(F)cc(OC3CCC4(CCNC4)CC3)cc2F)N1CC(F)(F)F] | 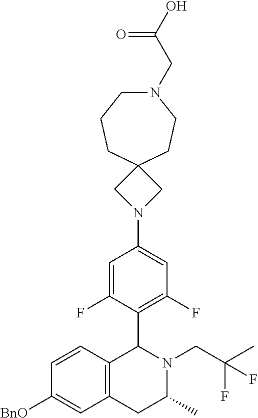 [SMILES: C[C@@H]1Cc2cc(OCc3ccccc3)ccc2C(c2c(F)cc(N3CC4(CCCN(CC(=O)O)CC4)C3)cc2F)N1CC(C)(F)F] | 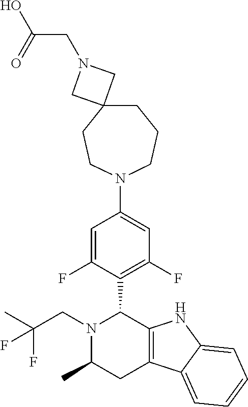 [SMILES: C[C@@H]1Cc2c([nH]c3ccccc23)[C@@H](c2c(F)cc(N3CCCC4(CC3)CN(CC(=O)O)C4)cc2F)N1CC(C)(F)F] |
| 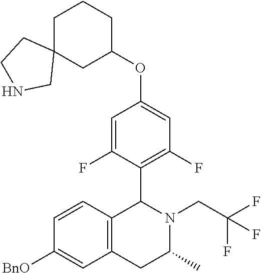 [SMILES: C[C@@H]1Cc2cc(OCc3ccccc3)ccc2C(c2c(F)cc(OC3CCCC4(CCNC4)C3)cc2F)N1CC(F)(F)F] | 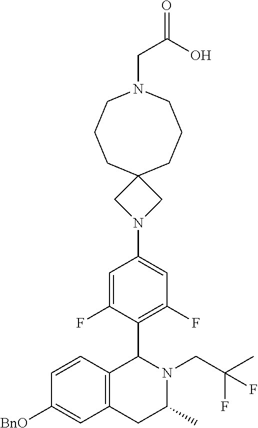 [SMILES: C[C@@H]1Cc2cc(OCc3ccccc3)ccc2C(c2c(F)cc(N3CC4(CCCN(CC(=O)O)CCC4)C3)cc2F)N1CC(C)(F)F] | 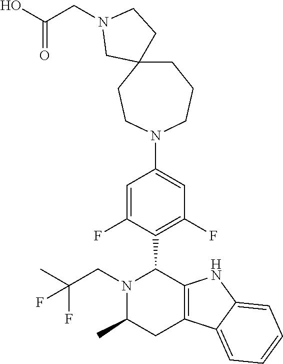 [SMILES: C[C@@H]1Cc2c([nH]c3ccccc23)[C@@H](c2c(F)cc(N3CCCC4(CCN(CC(=O)O)C4)CC3)cc2F)N1CC(C)(F)F] |
| 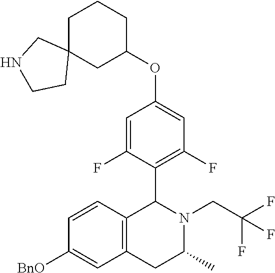 [SMILES: C[C@@H]1Cc2cc(OCc3ccccc3)ccc2C(c2c(F)cc(OC3CCCC4(CCNC4)C3)cc2F)N1CC(F)(F)F] | 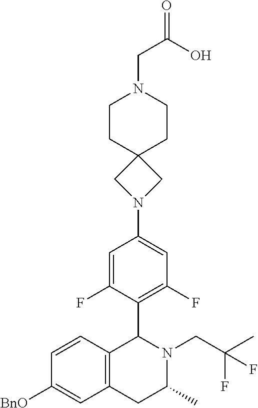 [SMILES: C[C@@H]1Cc2cc(OCc3ccccc3)ccc2C(c2c(F)cc(N3CC4(CCN(CC(=O)O)CC4)C3)cc2F)N1CC(C)(F)F] | 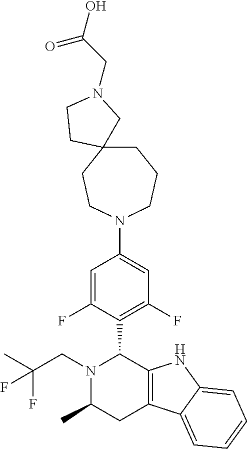 [SMILES: C[C@@H]1Cc2c([nH]c3ccccc23)[C@@H](c2c(F)cc(N3CCCC4(CCN(CC(=O)O)C4)CC3)cc2F)N1CC(C)(F)F] |
| 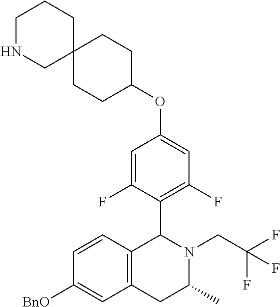 [SMILES: C[C@@H]1Cc2cc(OCc3ccccc3)ccc2C(c2c(F)cc(OC3CCC4(CCCNC4)CC3)cc2F)N1CC(F)(F)F] | 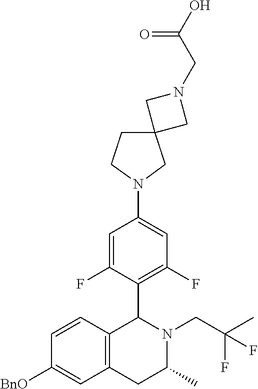 [SMILES: C[C@@H]1Cc2cc(OCc3ccccc3)ccc2C(c2c(F)cc(N3CCC4(CN(CC(=O)O)C4)C3)cc2F)N1CC(C)(F)F] | 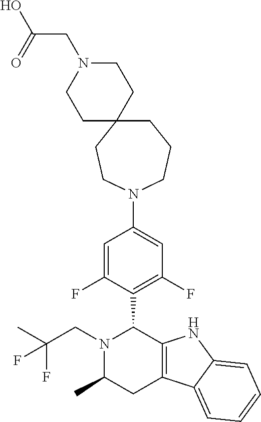 [SMILES: C[C@@H]1Cc2c([nH]c3ccccc23)[C@@H](c2c(F)cc(N3CCCC4(CCN(CC(=O)O)CC4)CC3)cc2F)N1CC(C)(F)F] |
| 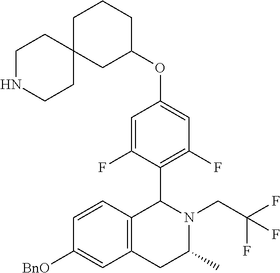 [SMILES: C[C@@H]1Cc2cc(OCc3ccccc3)ccc2C(c2c(F)cc(OC3CCCC4(CCNCC4)C3)cc2F)N1CC(F)(F)F] | 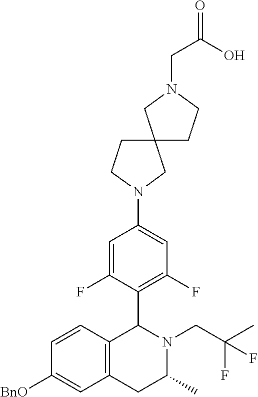 [SMILES: C[C@@H]1Cc2cc(OCc3ccccc3)ccc2C(c2c(F)cc(N3CCC4(CCN(CC(=O)O)C4)C3)cc2F)N1CC(C)(F)F] | 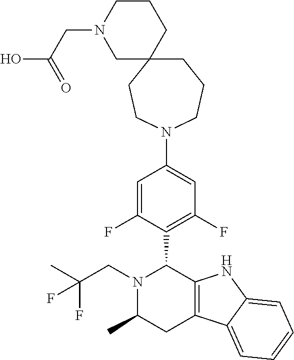 [SMILES: C[C@@H]1Cc2c([nH]c3ccccc23)[C@@H](c2c(F)cc(N3CCCC4(CCCN(CC(=O)O)C4)CC3)cc2F)N1CC(C)(F)F] |
| 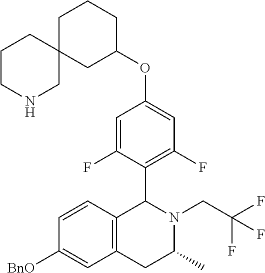 [SMILES: C[C@@H]1Cc2cc(OCc3ccccc3)ccc2C(c2c(F)cc(OC3CCCC4(CCCNC4)C3)cc2F)N1CC(F)(F)F] | 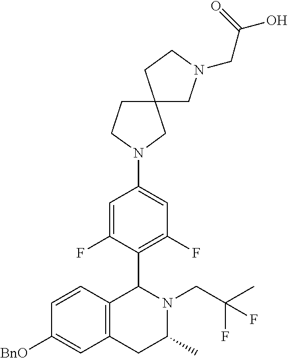 [SMILES: C[C@@H]1Cc2cc(OCc3ccccc3)ccc2C(c2c(F)cc(N3CCC4(CCN(CC(=O)O)C4)C3)cc2F)N1CC(C)(F)F] | 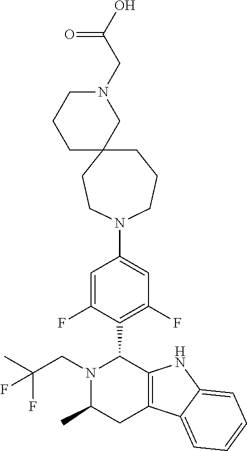 [SMILES: C[C@@H]1Cc2c([nH]c3ccccc23)[C@@H](c2c(F)cc(N3CCCC4(CCCN(CC(=O)O)C4)CC3)cc2F)N1CC(C)(F)F] |
| 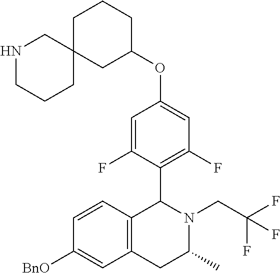 [SMILES: C[C@@H]1Cc2cc(OCc3ccccc3)ccc2C(c2c(F)cc(OC3CCCC4(CCCNC4)C3)cc2F)N1CC(F)(F)F] | 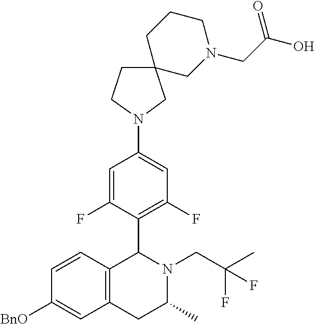 [SMILES: C[C@@H]1Cc2cc(OCc3ccccc3)ccc2C(c2c(F)cc(N3CCC4(CCCN(CC(=O)O)C4)C3)cc2F)N1CC(C)(F)F] | 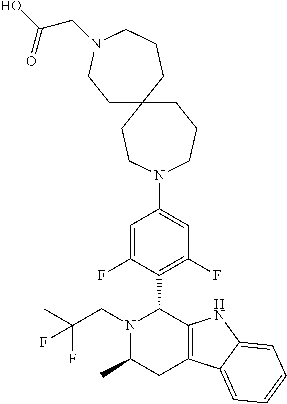 [SMILES: C[C@@H]1Cc2c([nH]c3ccccc23)[C@@H](c2c(F)cc(N3CCCC4(CCCN(CC(=O)O)CC4)CC3)cc2F)N1CC(C)(F)F] |
| 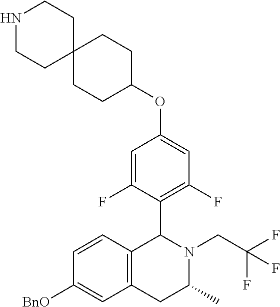 [SMILES: C[C@@H]1Cc2cc(OCc3ccccc3)ccc2C(c2c(F)cc(OC3CCC4(CCNCC4)CC3)cc2F)N1CC(F)(F)F] | 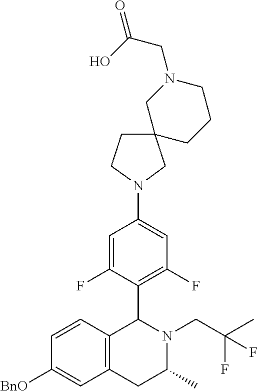 [SMILES: C[C@@H]1Cc2cc(OCc3ccccc3)ccc2C(c2c(F)cc(N3CCC4(CCCN(CC(=O)O)C4)C3)cc2F)N1CC(C)(F)F] | 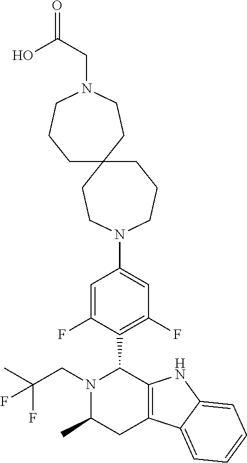 [SMILES: C[C@@H]1Cc2c([nH]c3ccccc23)[C@@H](c2c(F)cc(N3CCCC4(CCCN(CC(=O)O)CC4)CC3)cc2F)N1CC(C)(F)F] |
| 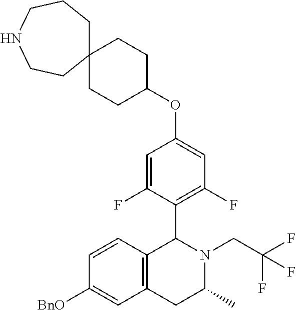 [SMILES: C[C@@H]1Cc2cc(OCc3ccccc3)ccc2C(c2c(F)cc(OC3CCC4(CCCNCC4)CC3)cc2F)N1CC(F)(F)F] | 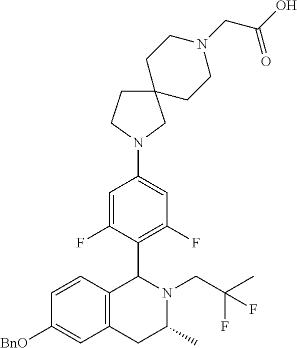 [SMILES: C[C@@H]1Cc2cc(OCc3ccccc3)ccc2C(c2c(F)cc(N3CCC4(CCN(CC(=O)O)CC4)C3)cc2F)N1CC(C)(F)F] | 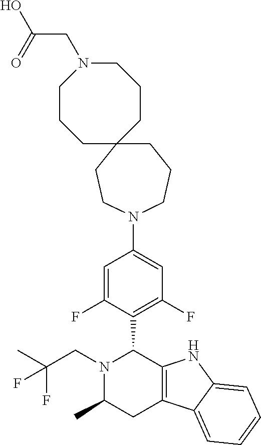 [SMILES: C[C@@H]1Cc2c([nH]c3ccccc23)[C@@H](c2c(F)cc(N3CCCC4(CCCN(CC(=O)O)CCC4)CC3)cc2F)N1CC(C)(F)F] |
| 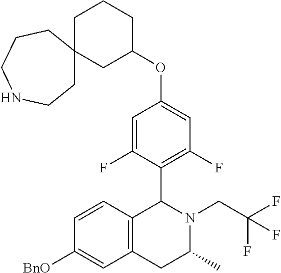 [SMILES: C[C@@H]1Cc2cc(OCc3ccccc3)ccc2C(c2c(F)cc(OC3CCCC4(CCCNCC4)C3)cc2F)N1CC(F)(F)F] | 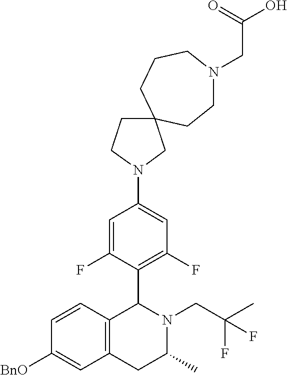 [SMILES: C[C@@H]1Cc2cc(OCc3ccccc3)ccc2C(c2c(F)cc(N3CCC4(CCCN(CC(=O)O)CC4)C3)cc2F)N1CC(C)(F)F] | 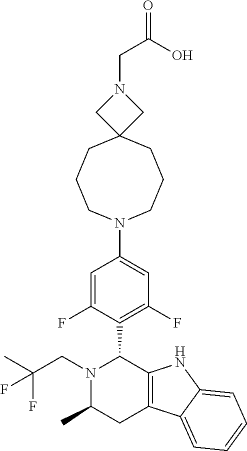 [SMILES: C[C@@H]1Cc2c([nH]c3ccccc23)[C@@H](c2c(F)cc(N3CCCC4(CCC3)CN(CC(=O)O)C4)cc2F)N1CC(C)(F)F] |
| 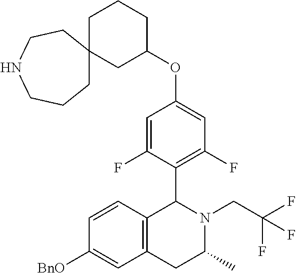 [SMILES: C[C@@H]1Cc2cc(OCc3ccccc3)ccc2C(c2c(F)cc(OC3CCCC4(CCCNCC4)C3)cc2F)N1CC(F)(F)F] | 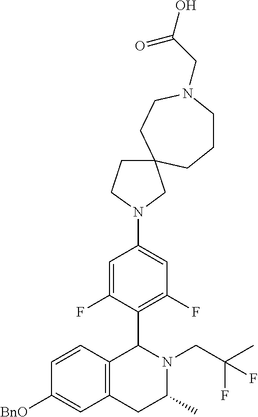 [SMILES: C[C@@H]1Cc2cc(OCc3ccccc3)ccc2C(c2c(F)cc(N3CCC4(CCCN(CC(=O)O)CC4)C3)cc2F)N1CC(C)(F)F] | 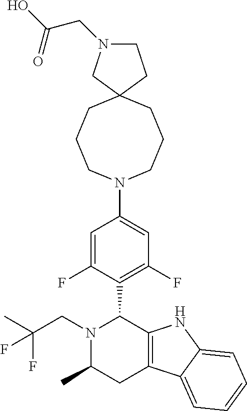 [SMILES: C[C@@H]1Cc2c([nH]c3ccccc23)[C@@H](c2c(F)cc(N3CCCC4(CCC3)CCN(CC(=O)O)C4)cc2F)N1CC(C)(F)F] |
| 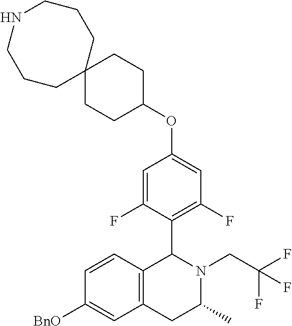 [SMILES: C[C@@H]1Cc2cc(OCc3ccccc3)ccc2C(c2c(F)cc(OC3CCC4(CCCNCCC4)CC3)cc2F)N1CC(F)(F)F] | 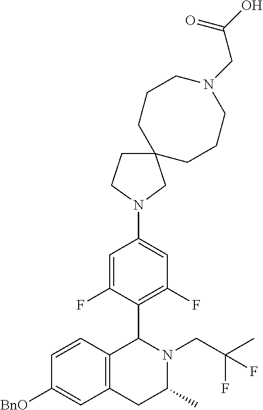 [SMILES: C[C@@H]1Cc2cc(OCc3ccccc3)ccc2C(c2c(F)cc(N3CCC4(CCCN(CC(=O)O)CCC4)C3)cc2F)N1CC(C)(F)F] | 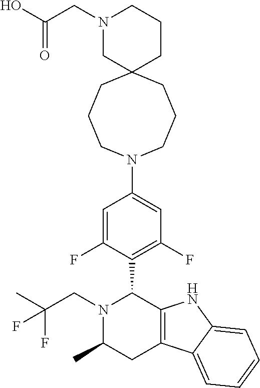 [SMILES: C[C@@H]1Cc2c([nH]c3ccccc23)[C@@H](c2c(F)cc(N3CCCC4(CCCN(CC(=O)O)C4)CCC3)cc2F)N1CC(C)(F)F] |
| 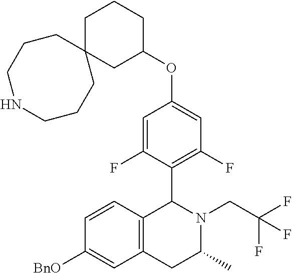 [SMILES: C[C@@H]1Cc2cc(OCc3ccccc3)ccc2C(c2c(F)cc(OC3CCCC4(CCCNCCC4)C3)cc2F)N1CC(F)(F)F] | 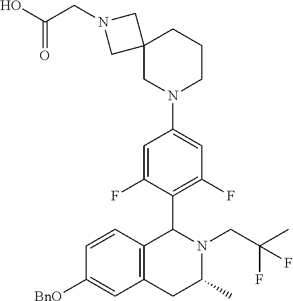 [SMILES: C[C@@H]1Cc2cc(OCc3ccccc3)ccc2C(c2c(F)cc(N3CCCC4(CN(CC(=O)O)C4)C3)cc2F)N1CC(C)(F)F] | 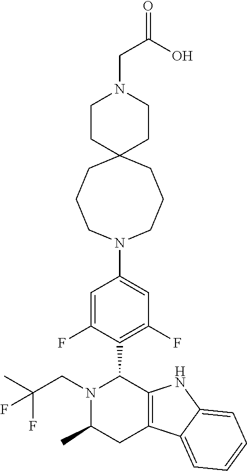 [SMILES: C[C@@H]1Cc2c([nH]c3ccccc23)[C@@H](c2c(F)cc(N3CCCC4(CCC3)CCN(CC(=O)O)CC4)cc2F)N1CC(C)(F)F] |
| 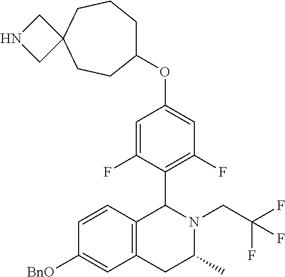 [SMILES: C[C@@H]1Cc2cc(OCc3ccccc3)ccc2C(c2c(F)cc(OC3CCCC4(CC3)CNC4)cc2F)N1CC(F)(F)F] | 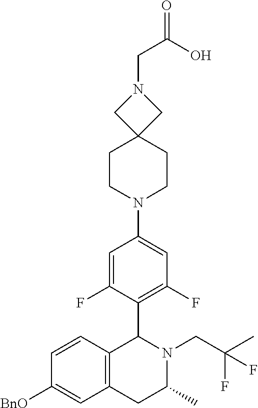 [SMILES: C[C@@H]1Cc2cc(OCc3ccccc3)ccc2C(c2c(F)cc(N3CCC4(CC3)CN(CC(=O)O)C4)cc2F)N1CC(C)(F)F] | 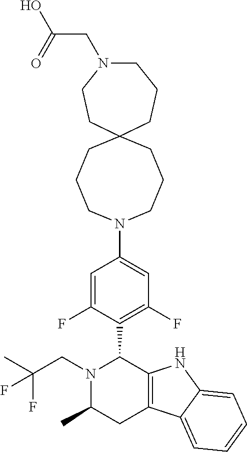 [SMILES: C[C@@H]1Cc2c([nH]c3ccccc23)[C@@H](c2c(F)cc(N3CCCC4(CCCN(CC(=O)O)CC4)CCC3)cc2F)N1CC(C)(F)F] |
| 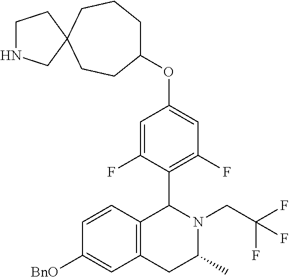 [SMILES: C[C@@H]1Cc2cc(OCc3ccccc3)ccc2C(c2c(F)cc(OC3CCCC4(CCNC4)CC3)cc2F)N1CC(F)(F)F] | 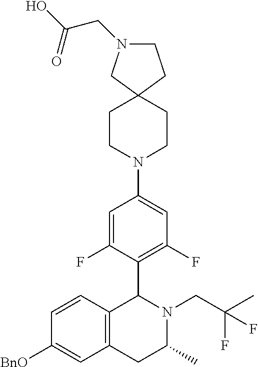 [SMILES: C[C@@H]1Cc2cc(OCc3ccccc3)ccc2C(c2c(F)cc(N3CCC4(CCN(CC(=O)O)C4)CC3)cc2F)N1CC(C)(F)F] | 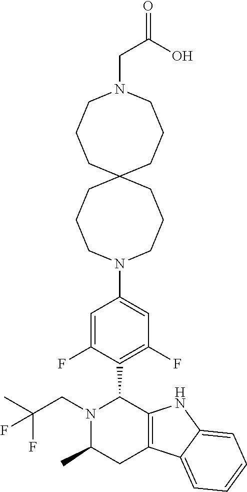 [SMILES: C[C@@H]1Cc2c([nH]c3ccccc23)[C@@H](c2c(F)cc(N3CCCC4(CCCN(CC(=O)O)CCC4)CCC3)cc2F)N1CC(C)(F)F] |
| 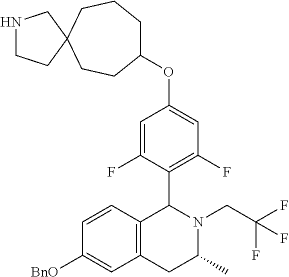 [SMILES: C[C@@H]1Cc2cc(OCc3ccccc3)ccc2C(c2c(F)cc(OC3CCCC4(CCNC4)CC3)cc2F)N1CC(F)(F)F] | 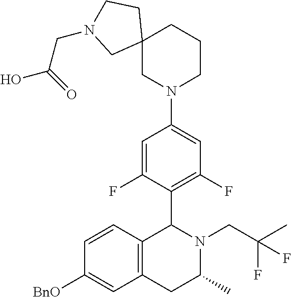 [SMILES: C[C@@H]1Cc2cc(OCc3ccccc3)ccc2C(c2c(F)cc(N3CCCC4(CCN(CC(=O)O)C4)C3)cc2F)N1CC(C)(F)F] | 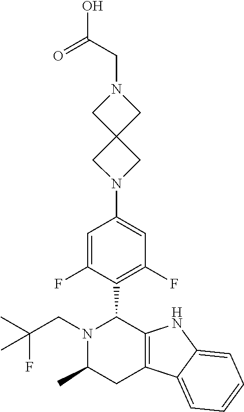 [SMILES: C[C@@H]1Cc2c([nH]c3ccccc23)[C@@H](c2c(F)cc(N3CC4(CN(CC(=O)O)C4)C3)cc2F)N1CC(C)(C)F] |
| 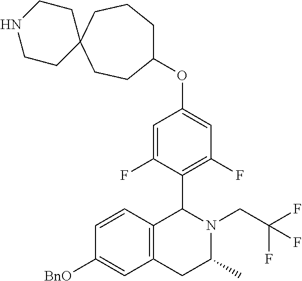 [SMILES: C[C@@H]1Cc2cc(OCc3ccccc3)ccc2C(c2c(F)cc(OC3CCCC4(CCNCC4)CC3)cc2F)N1CC(F)(F)F] | 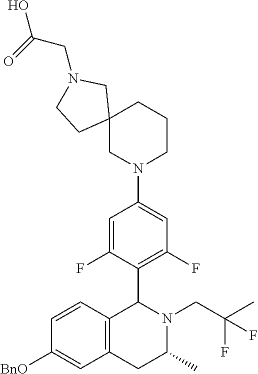 [SMILES: C[C@@H]1Cc2cc(OCc3ccccc3)ccc2C(c2c(F)cc(N3CCCC4(CCN(CC(=O)O)C4)C3)cc2F)N1CC(C)(F)F] | 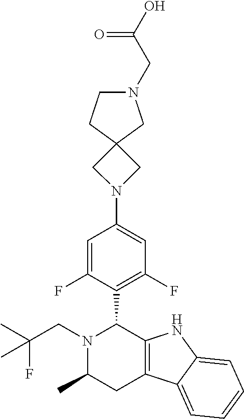 [SMILES: C[C@@H]1Cc2c([nH]c3ccccc23)[C@@H](c2c(F)cc(N3CC4(CCN(CC(=O)O)C4)C3)cc2F)N1CC(C)(C)F] |
| 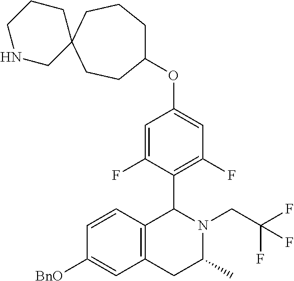 [SMILES: C[C@@H]1Cc2cc(OCc3ccccc3)ccc2C(c2c(F)cc(OC3CCCC4(CCCNC4)CC3)cc2F)N1CC(F)(F)F] | 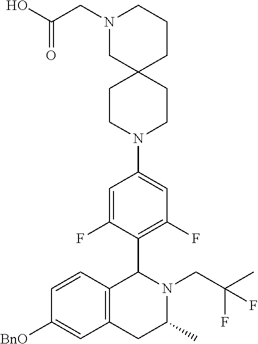 [SMILES: C[C@@H]1Cc2cc(OCc3ccccc3)ccc2C(c2c(F)cc(N3CCC4(CCCN(CC(=O)O)C4)CC3)cc2F)N1CC(C)(F)F] | 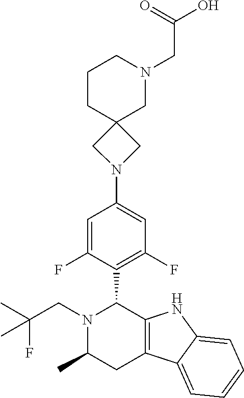 [SMILES: C[C@@H]1Cc2c([nH]c3ccccc23)[C@@H](c2c(F)cc(N3CC4(CCCN(CC(=O)O)C4)C3)cc2F)N1CC(C)(C)F] |
| 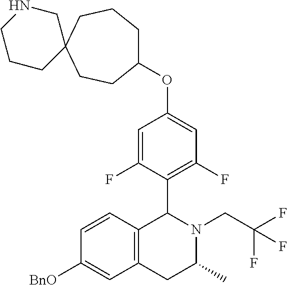 [SMILES: C[C@@H]1Cc2cc(OCc3ccccc3)ccc2C(c2c(F)cc(OC3CCCC4(CCCNC4)CC3)cc2F)N1CC(F)(F)F] | 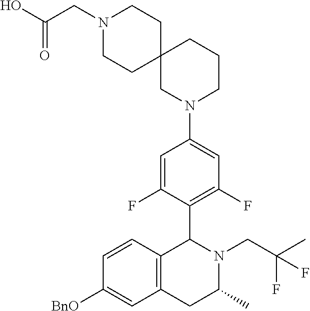 [SMILES: C[C@@H]1Cc2cc(OCc3ccccc3)ccc2C(c2c(F)cc(N3CCCC4(CCN(CC(=O)O)CC4)C3)cc2F)N1CC(C)(F)F] | 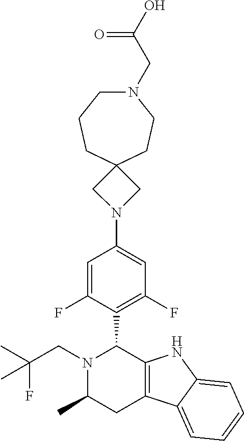 [SMILES: C[C@@H]1Cc2c([nH]c3ccccc23)[C@@H](c2c(F)cc(N3CC4(CCCN(CC(=O)O)CC4)C3)cc2F)N1CC(C)(C)F] |
| 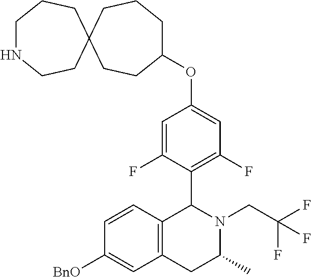 [SMILES: C[C@@H]1Cc2cc(OCc3ccccc3)ccc2C(c2c(F)cc(OC3CCCC4(CCCNCC4)CC3)cc2F)N1CC(F)(F)F] | 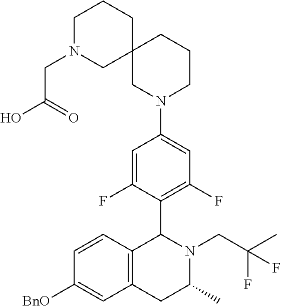 [SMILES: C[C@@H]1Cc2cc(OCc3ccccc3)ccc2C(c2c(F)cc(N3CCCC4(CCCN(CC(=O)O)C4)C3)cc2F)N1CC(C)(F)F] | 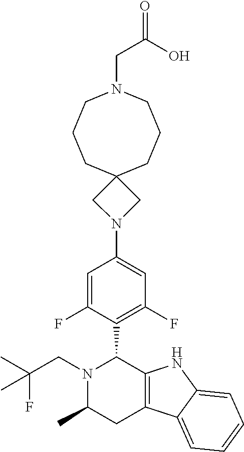 [SMILES: C[C@@H]1Cc2c([nH]c3ccccc23)[C@@H](c2c(F)cc(N3CC4(CCCN(CC(=O)O)CCC4)C3)cc2F)N1CC(C)(C)F] |
| 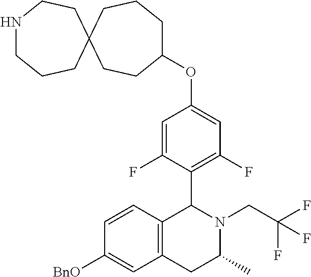 [SMILES: C[C@@H]1Cc2cc(OCc3ccccc3)ccc2C(c2c(F)cc(OC3CCCC4(CCCNCC4)CC3)cc2F)N1CC(F)(F)F] | 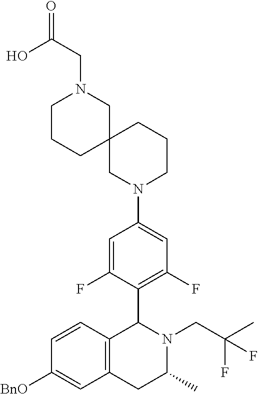 [SMILES: C[C@@H]1Cc2cc(OCc3ccccc3)ccc2C(c2c(F)cc(N3CCCC4(CCCN(CC(=O)O)C4)C3)cc2F)N1CC(C)(F)F] | 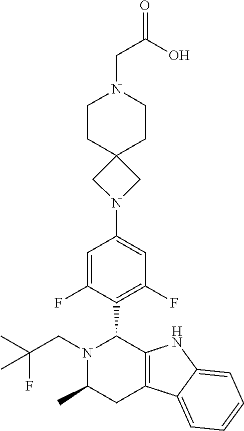 [SMILES: C[C@@H]1Cc2c([nH]c3ccccc23)[C@@H](c2c(F)cc(N3CC4(CCN(CC(=O)O)CC4)C3)cc2F)N1CC(C)(C)F] |
| 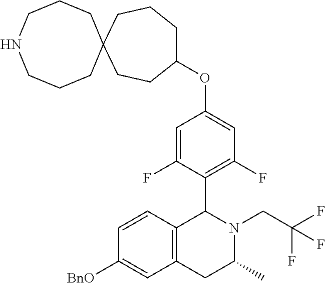 [SMILES: C[C@@H]1Cc2cc(OCc3ccccc3)ccc2C(c2c(F)cc(OC3CCCC4(CCCNCCC4)CC3)cc2F)N1CC(F)(F)F] | 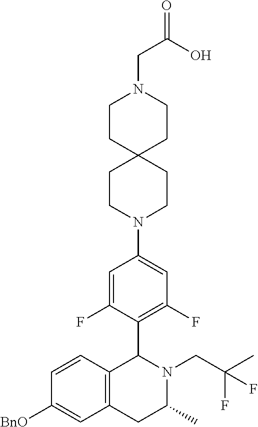 [SMILES: C[C@@H]1Cc2cc(OCc3ccccc3)ccc2C(c2c(F)cc(N3CCC4(CCN(CC(=O)O)CC4)CC3)cc2F)N1CC(C)(F)F] | 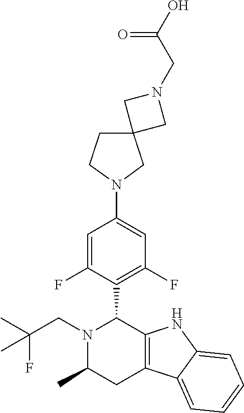 [SMILES: C[C@@H]1Cc2c([nH]c3ccccc23)[C@@H](c2c(F)cc(N3CCC4(CN(CC(=O)O)C4)C3)cc2F)N1CC(C)(C)F] |
| 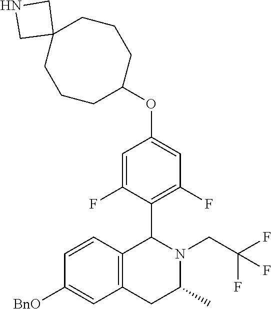 [SMILES: C[C@@H]1Cc2cc(OCc3ccccc3)ccc2C(c2c(F)cc(OC3CCCC4(CCC3)CNC4)cc2F)N1CC(F)(F)F] | 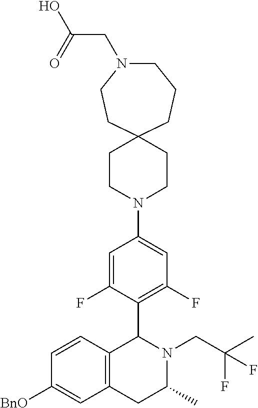 [SMILES: C[C@@H]1Cc2cc(OCc3ccccc3)ccc2C(c2c(F)cc(N3CCC4(CCCN(CC(=O)O)CC4)CC3)cc2F)N1CC(C)(F)F] | 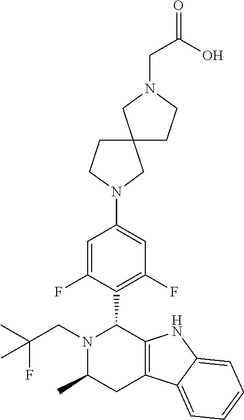 [SMILES: C[C@@H]1Cc2c([nH]c3ccccc23)[C@@H](c2c(F)cc(N3CCC4(CCN(CC(=O)O)C4)C3)cc2F)N1CC(C)(C)F] |
| 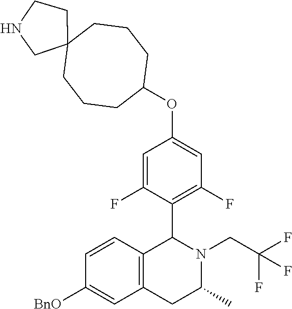 [SMILES: C[C@@H]1Cc2cc(OCc3ccccc3)ccc2C(c2c(F)cc(OC3CCCC4(CCC3)CCNC4)cc2F)N1CC(F)(F)F] | 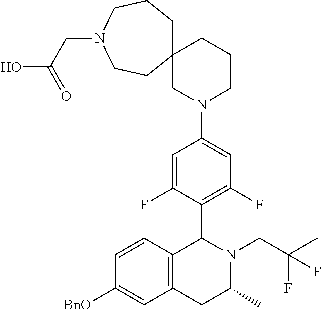 [SMILES: C[C@@H]1Cc2cc(OCc3ccccc3)ccc2C(c2c(F)cc(N3CCCC4(CCCN(CC(=O)O)CC4)C3)cc2F)N1CC(C)(F)F] | 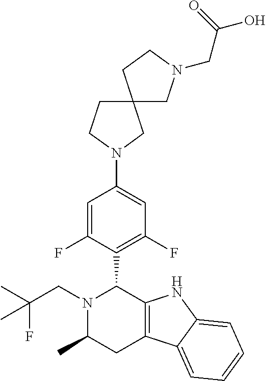 [SMILES: C[C@@H]1Cc2c([nH]c3ccccc23)[C@@H](c2c(F)cc(N3CCC4(CCN(CC(=O)O)C4)C3)cc2F)N1CC(C)(C)F] |
| 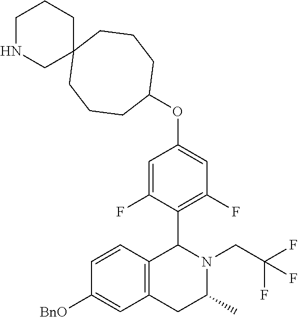 [SMILES: C[C@@H]1Cc2cc(OCc3ccccc3)ccc2C(c2c(F)cc(OC3CCCC4(CCCNC4)CCC3)cc2F)N1CC(F)(F)F] | 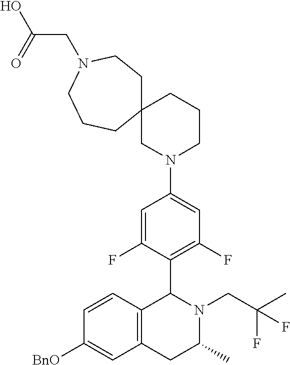 [SMILES: C[C@@H]1Cc2cc(OCc3ccccc3)ccc2C(c2c(F)cc(N3CCCC4(CCCN(CC(=O)O)CC4)C3)cc2F)N1CC(C)(F)F] | 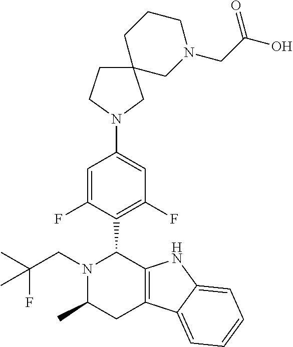 [SMILES: C[C@@H]1Cc2c([nH]c3ccccc23)[C@@H](c2c(F)cc(N3CCC4(CCCN(CC(=O)O)C4)C3)cc2F)N1CC(C)(C)F] |
| 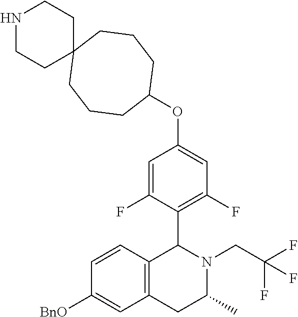 [SMILES: C[C@@H]1Cc2cc(OCc3ccccc3)ccc2C(c2c(F)cc(OC3CCCC4(CCC3)CCNCC4)cc2F)N1CC(F)(F)F] | 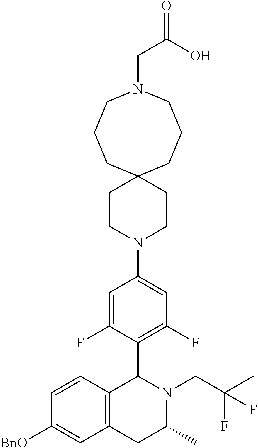 [SMILES: C[C@@H]1Cc2cc(OCc3ccccc3)ccc2C(c2c(F)cc(N3CCC4(CCCN(CC(=O)O)CCC4)CC3)cc2F)N1CC(C)(F)F] | 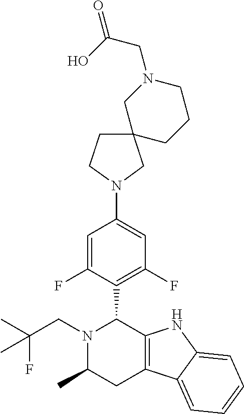 [SMILES: C[C@@H]1Cc2c([nH]c3ccccc23)[C@@H](c2c(F)cc(N3CCC4(CCCN(CC(=O)O)C4)C3)cc2F)N1CC(C)(C)F] |
| 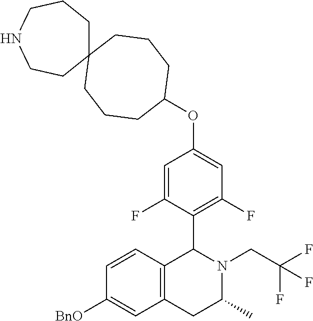 [SMILES: C[C@@H]1Cc2cc(OCc3ccccc3)ccc2C(c2c(F)cc(OC3CCCC4(CCCNCC4)CCC3)cc2F)N1CC(F)(F)F] | 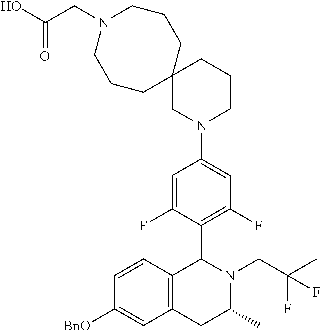 [SMILES: C[C@@H]1Cc2cc(OCc3ccccc3)ccc2C(c2c(F)cc(N3CCCC4(CCCN(CC(=O)O)CCC4)C3)cc2F)N1CC(C)(F)F] | 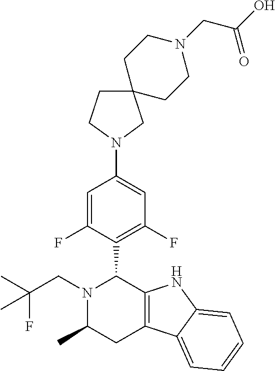 [SMILES: C[C@@H]1Cc2c([nH]c3ccccc23)[C@@H](c2c(F)cc(N3CCC4(CCN(CC(=O)O)CC4)C3)cc2F)N1CC(C)(C)F] |
| 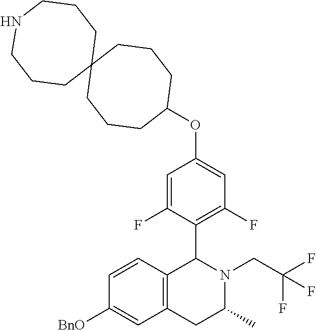 [SMILES: C[C@@H]1Cc2cc(OCc3ccccc3)ccc2C(c2c(F)cc(OC3CCCC4(CCCNCCC4)CCC3)cc2F)N1CC(F)(F)F] | 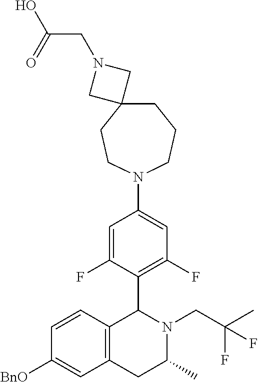 [SMILES: C[C@@H]1Cc2cc(OCc3ccccc3)ccc2C(c2c(F)cc(N3CCCC4(CC3)CN(CC(=O)O)C4)cc2F)N1CC(C)(F)F] | 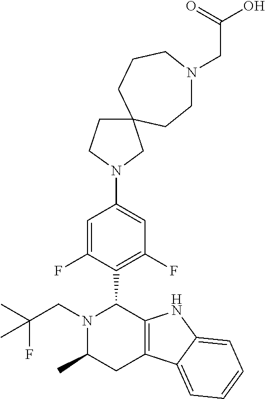 [SMILES: C[C@@H]1Cc2c([nH]c3ccccc23)[C@@H](c2c(F)cc(N3CCC4(CCCN(CC(=O)O)CC4)C3)cc2F)N1CC(C)(C)F] |
| 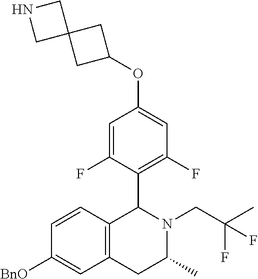 [SMILES: C[C@@H]1Cc2cc(OCc3ccccc3)ccc2C(c2c(F)cc(OC3CC4(CNC4)C3)cc2F)N1CC(C)(F)F] | 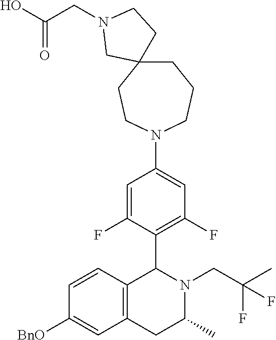 [SMILES: C[C@@H]1Cc2cc(OCc3ccccc3)ccc2C(c2c(F)cc(N3CCCC4(CCN(CC(=O)O)C4)CC3)cc2F)N1CC(C)(F)F] | 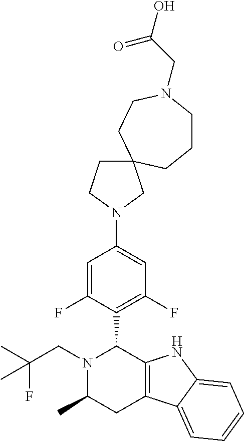 [SMILES: C[C@@H]1Cc2c([nH]c3ccccc23)[C@@H](c2c(F)cc(N3CCC4(CCCN(CC(=O)O)CC4)C3)cc2F)N1CC(C)(C)F] |
| 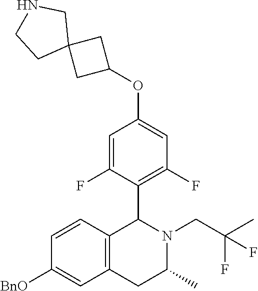 [SMILES: C[C@@H]1Cc2cc(OCc3ccccc3)ccc2C(c2c(F)cc(OC3CC4(CCNC4)C3)cc2F)N1CC(C)(F)F] | 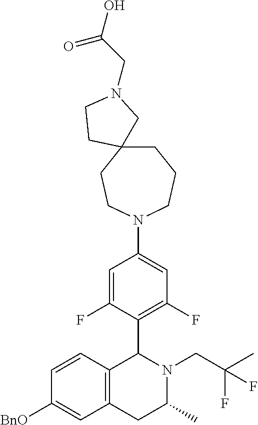 [SMILES: C[C@@H]1Cc2cc(OCc3ccccc3)ccc2C(c2c(F)cc(N3CCCC4(CCN(CC(=O)O)C4)CC3)cc2F)N1CC(C)(F)F] | 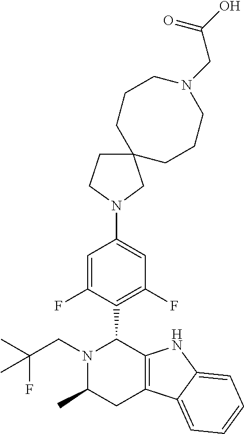 [SMILES: C[C@@H]1Cc2c([nH]c3ccccc23)[C@@H](c2c(F)cc(N3CCC4(CCCN(CC(=O)O)CCC4)C3)cc2F)N1CC(C)(C)F] |
| 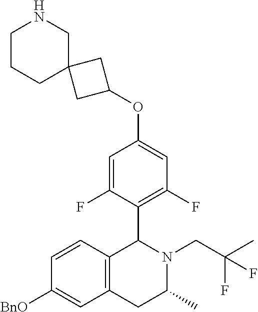 [SMILES: C[C@@H]1Cc2cc(OCc3ccccc3)ccc2C(c2c(F)cc(OC3CC4(CCCNC4)C3)cc2F)N1CC(C)(F)F] | 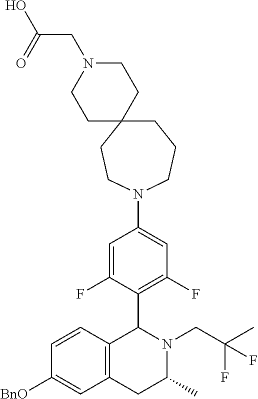 [SMILES: C[C@@H]1Cc2cc(OCc3ccccc3)ccc2C(c2c(F)cc(N3CCCC4(CCN(CC(=O)O)CC4)CC3)cc2F)N1CC(C)(F)F] | 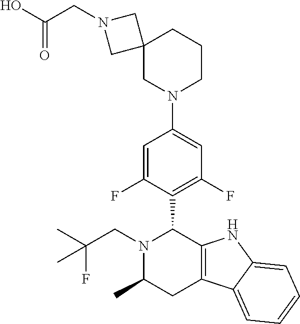 [SMILES: C[C@@H]1Cc2c([nH]c3ccccc23)[C@@H](c2c(F)cc(N3CCCC4(CN(CC(=O)O)C4)C3)cc2F)N1CC(C)(C)F] |
| 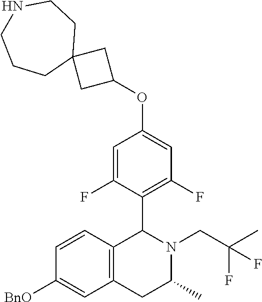 [SMILES: C[C@@H]1Cc2cc(OCc3ccccc3)ccc2C(c2c(F)cc(OC3CC4(CCCNCC4)C3)cc2F)N1CC(C)(F)F] | 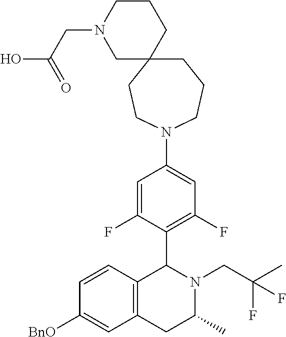 [SMILES: C[C@@H]1Cc2cc(OCc3ccccc3)ccc2C(c2c(F)cc(N3CCCC4(CCCN(CC(=O)O)C4)CC3)cc2F)N1CC(C)(F)F] | 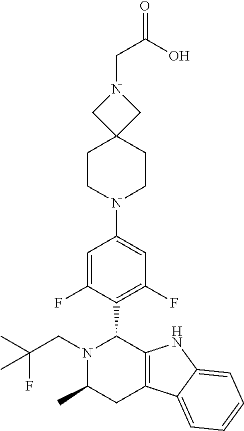 [SMILES: C[C@@H]1Cc2c([nH]c3ccccc23)[C@@H](c2c(F)cc(N3CCC4(CC3)CN(CC(=O)O)C4)cc2F)N1CC(C)(C)F] |
| 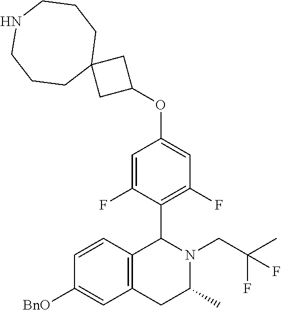 [SMILES: C[C@@H]1Cc2cc(OCc3ccccc3)ccc2C(c2c(F)cc(OC3CC4(CCCNCCC4)C3)cc2F)N1CC(C)(F)F] | 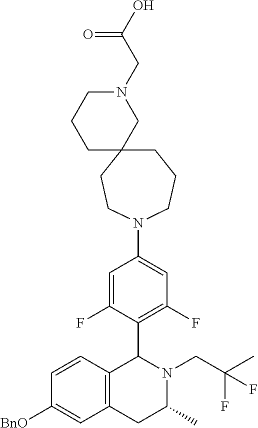 [SMILES: C[C@@H]1Cc2cc(OCc3ccccc3)ccc2C(c2c(F)cc(N3CCCC4(CCCN(CC(=O)O)C4)CC3)cc2F)N1CC(C)(F)F] | 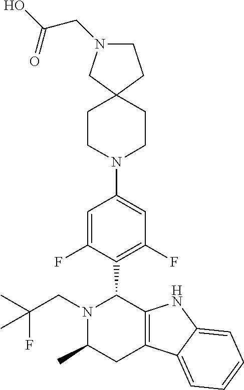 [SMILES: C[C@@H]1Cc2c([nH]c3ccccc23)[C@@H](c2c(F)cc(N3CCC4(CCN(CC(=O)O)C4)CC3)cc2F)N1CC(C)(C)F] |
| 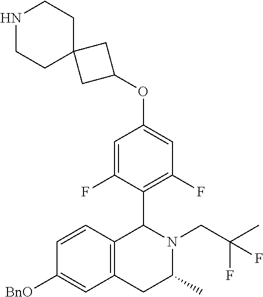 [SMILES: C[C@@H]1Cc2cc(OCc3ccccc3)ccc2C(c2c(F)cc(OC3CC4(CCNCC4)C3)cc2F)N1CC(C)(F)F] | 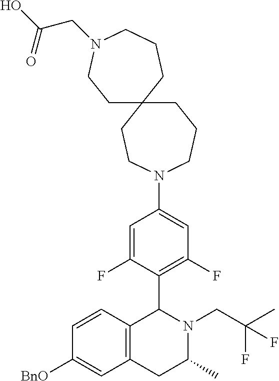 [SMILES: C[C@@H]1Cc2cc(OCc3ccccc3)ccc2C(c2c(F)cc(N3CCCC4(CCCN(CC(=O)O)CC4)CC3)cc2F)N1CC(C)(F)F] | 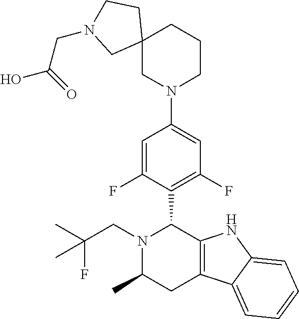 [SMILES: C[C@@H]1Cc2c([nH]c3ccccc23)[C@@H](c2c(F)cc(N3CCCC4(CCN(CC(=O)O)C4)C3)cc2F)N1CC(C)(C)F] |
| 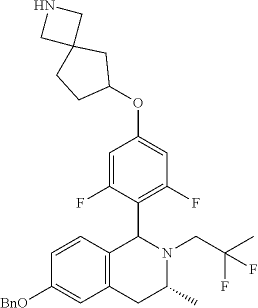 [SMILES: C[C@@H]1Cc2cc(OCc3ccccc3)ccc2C(c2c(F)cc(OC3CCC4(CNC4)C3)cc2F)N1CC(C)(F)F] | 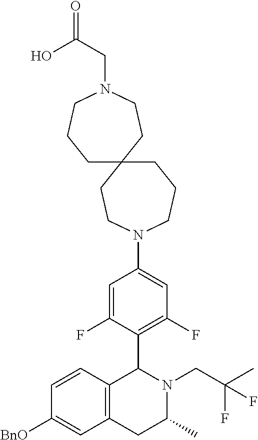 [SMILES: C[C@@H]1Cc2cc(OCc3ccccc3)ccc2C(c2c(F)cc(N3CCCC4(CCCN(CC(=O)O)CC4)CC3)cc2F)N1CC(C)(F)F] | 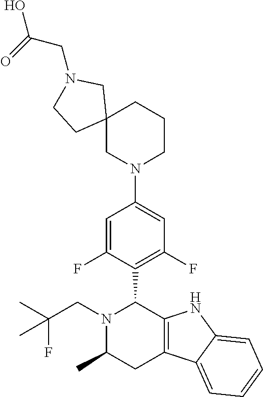 [SMILES: C[C@@H]1Cc2c([nH]c3ccccc23)[C@@H](c2c(F)cc(N3CCCC4(CCN(CC(=O)O)C4)C3)cc2F)N1CC(C)(C)F] |
| 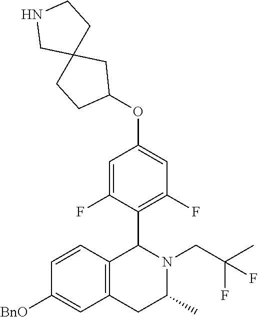 [SMILES: C[C@@H]1Cc2cc(OCc3ccccc3)ccc2C(c2c(F)cc(OC3CCC4(CCNC4)C3)cc2F)N1CC(C)(F)F] | 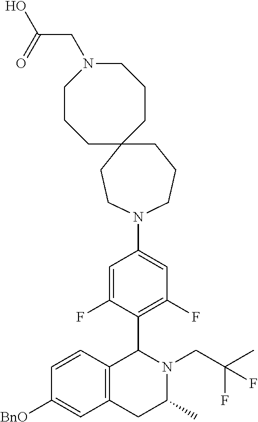 [SMILES: C[C@@H]1Cc2cc(OCc3ccccc3)ccc2C(c2c(F)cc(N3CCCC4(CCCN(CC(=O)O)CCC4)CC3)cc2F)N1CC(C)(F)F] | 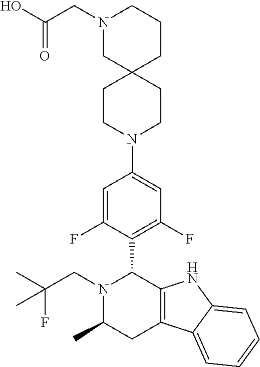 [SMILES: C[C@@H]1Cc2c([nH]c3ccccc23)[C@@H](c2c(F)cc(N3CCC4(CCCN(CC(=O)O)C4)CC3)cc2F)N1CC(C)(C)F] |
| 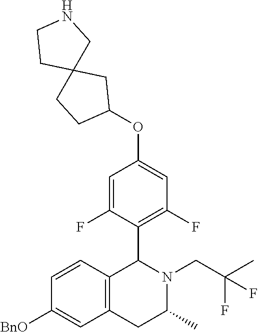 [SMILES: C[C@@H]1Cc2cc(OCc3ccccc3)ccc2C(c2c(F)cc(OC3CCC4(CCNC4)C3)cc2F)N1CC(C)(F)F] | 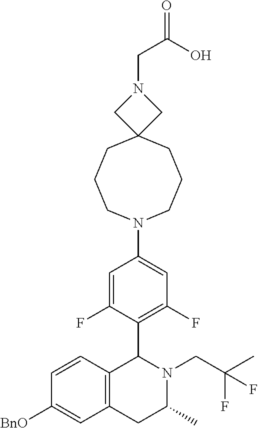 [SMILES: C[C@@H]1Cc2cc(OCc3ccccc3)ccc2C(c2c(F)cc(N3CCCC4(CCC3)CN(CC(=O)O)C4)cc2F)N1CC(C)(F)F] | 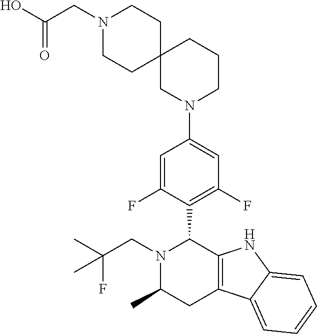 [SMILES: C[C@@H]1Cc2c([nH]c3ccccc23)[C@@H](c2c(F)cc(N3CCCC4(CCN(CC(=O)O)CC4)C3)cc2F)N1CC(C)(C)F] |
| 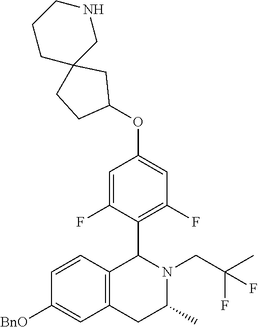 [SMILES: C[C@@H]1Cc2cc(OCc3ccccc3)ccc2C(c2c(F)cc(OC3CCC4(CCCNC4)C3)cc2F)N1CC(C)(F)F] | 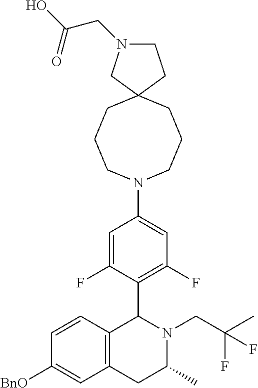 [SMILES: C[C@@H]1Cc2cc(OCc3ccccc3)ccc2C(c2c(F)cc(N3CCCC4(CCC3)CCN(CC(=O)O)C4)cc2F)N1CC(C)(F)F] | 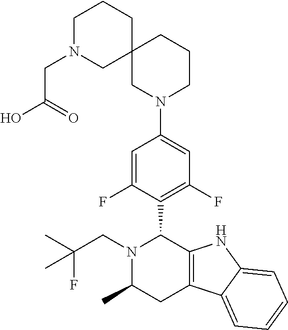 [SMILES: C[C@@H]1Cc2c([nH]c3ccccc23)[C@@H](c2c(F)cc(N3CCCC4(CCCN(CC(=O)O)C4)C3)cc2F)N1CC(C)(C)F] |
| 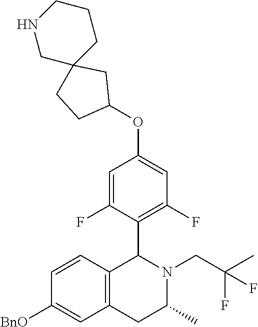 [SMILES: C[C@@H]1Cc2cc(OCc3ccccc3)ccc2C(c2c(F)cc(OC3CCC4(CCCNC4)C3)cc2F)N1CC(C)(F)F] | 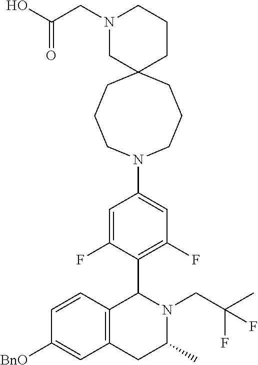 [SMILES: C[C@@H]1Cc2cc(OCc3ccccc3)ccc2C(c2c(F)cc(N3CCCC4(CCCN(CC(=O)O)C4)CCC3)cc2F)N1CC(C)(F)F] | 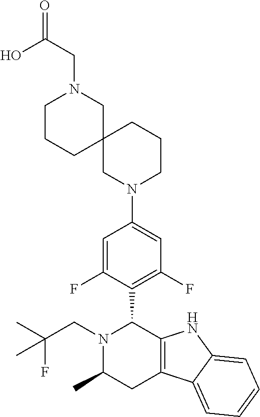 [SMILES: C[C@@H]1Cc2c([nH]c3ccccc23)[C@@H](c2c(F)cc(N3CCCC4(CCCN(CC(=O)O)C4)C3)cc2F)N1CC(C)(C)F] |
| 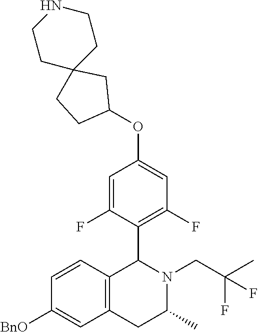 [SMILES: C[C@@H]1Cc2cc(OCc3ccccc3)ccc2C(c2c(F)cc(OC3CCC4(CCNCC4)C3)cc2F)N1CC(C)(F)F] | 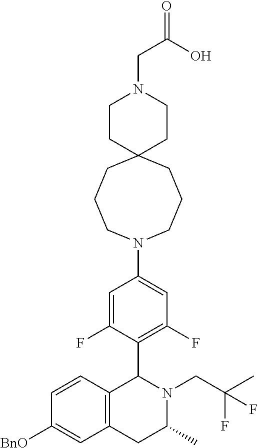 [SMILES: C[C@@H]1Cc2cc(OCc3ccccc3)ccc2C(c2c(F)cc(N3CCCC4(CCC3)CCN(CC(=O)O)CC4)cc2F)N1CC(C)(F)F] | 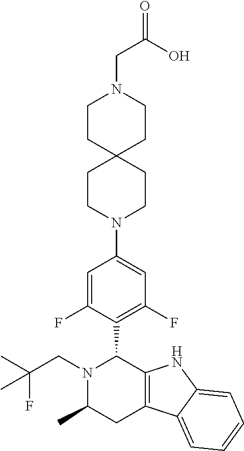 [SMILES: C[C@@H]1Cc2c([nH]c3ccccc23)[C@@H](c2c(F)cc(N3CCC4(CCN(CC(=O)O)CC4)CC3)cc2F)N1CC(C)(C)F] |
| 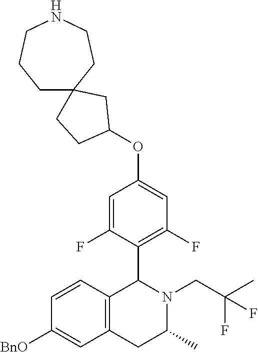 [SMILES: C[C@@H]1Cc2cc(OCc3ccccc3)ccc2C(c2c(F)cc(OC3CCC4(CCCNCC4)C3)cc2F)N1CC(C)(F)F] | 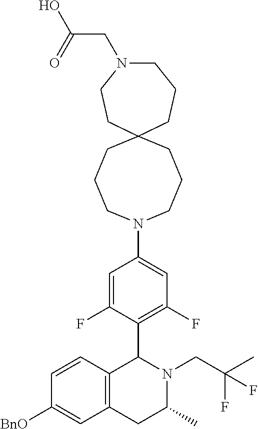 [SMILES: C[C@@H]1Cc2cc(OCc3ccccc3)ccc2C(c2c(F)cc(N3CCCC4(CCCN(CC(=O)O)CC4)CCC3)cc2F)N1CC(C)(F)F] | 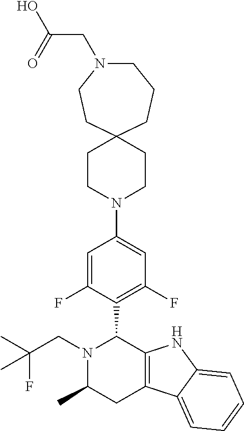 [SMILES: C[C@@H]1Cc2c([nH]c3ccccc23)[C@@H](c2c(F)cc(N3CCC4(CCCN(CC(=O)O)CC4)CC3)cc2F)N1CC(C)(C)F] |
| 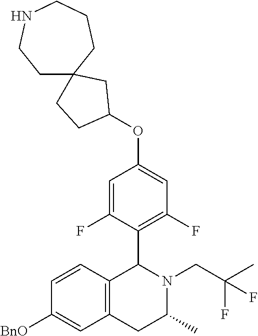 [SMILES: C[C@@H]1Cc2cc(OCc3ccccc3)ccc2C(c2c(F)cc(OC3CCC4(CCCNCC4)C3)cc2F)N1CC(C)(F)F] | 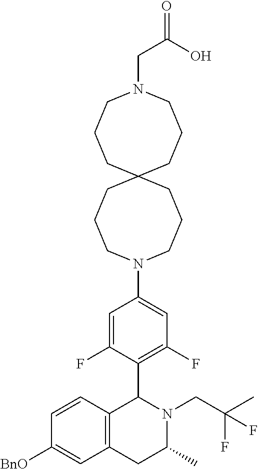 [SMILES: C[C@@H]1Cc2cc(OCc3ccccc3)ccc2C(c2c(F)cc(N3CCCC4(CCCN(CC(=O)O)CCC4)CCC3)cc2F)N1CC(C)(F)F] | 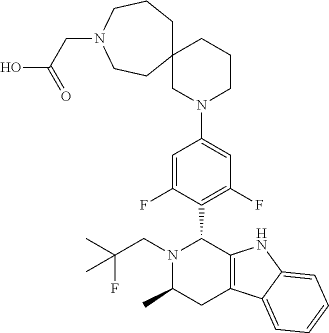 [SMILES: C[C@@H]1Cc2c([nH]c3ccccc23)[C@@H](c2c(F)cc(N3CCCC4(CCCN(CC(=O)O)CC4)C3)cc2F)N1CC(C)(C)F] |
| 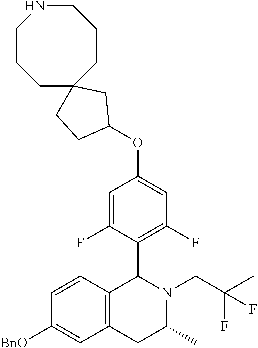 [SMILES: C[C@@H]1Cc2cc(OCc3ccccc3)ccc2C(c2c(F)cc(OC3CCC4(CCCNCCC4)C3)cc2F)N1CC(C)(F)F] | 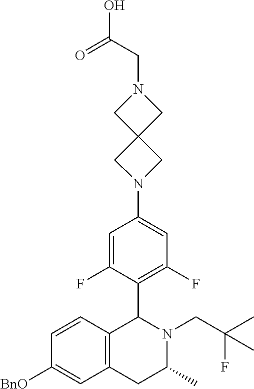 [SMILES: C[C@@H]1Cc2cc(OCc3ccccc3)ccc2C(c2c(F)cc(N3CC4(CN(CC(=O)O)C4)C3)cc2F)N1CC(C)(C)F] | 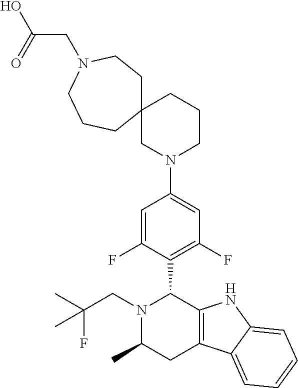 [SMILES: C[C@@H]1Cc2c([nH]c3ccccc23)[C@@H](c2c(F)cc(N3CCCC4(CCCN(CC(=O)O)CC4)C3)cc2F)N1CC(C)(C)F] |
| 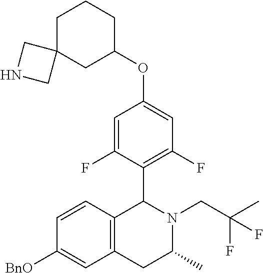 [SMILES: C[C@@H]1Cc2cc(OCc3ccccc3)ccc2C(c2c(F)cc(OC3CCCC4(CNC4)C3)cc2F)N1CC(C)(F)F] | 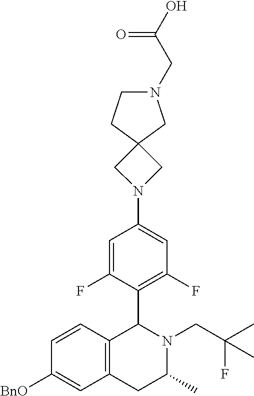 [SMILES: C[C@@H]1Cc2cc(OCc3ccccc3)ccc2C(c2c(F)cc(N3CC4(CCN(CC(=O)O)C4)C3)cc2F)N1CC(C)(C)F] | 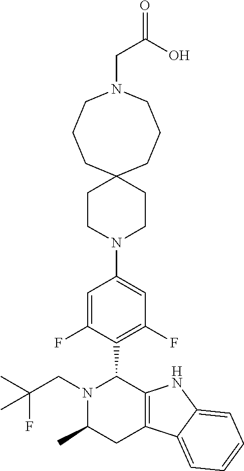 [SMILES: C[C@@H]1Cc2c([nH]c3ccccc23)[C@@H](c2c(F)cc(N3CCC4(CCCN(CC(=O)O)CCC4)CC3)cc2F)N1CC(C)(C)F] |
| 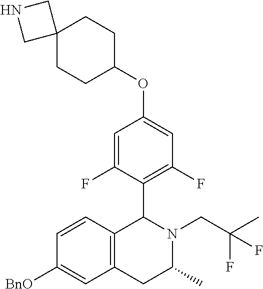 [SMILES: C[C@@H]1Cc2cc(OCc3ccccc3)ccc2C(c2c(F)cc(OC3CCC4(CC3)CNC4)cc2F)N1CC(C)(F)F] | 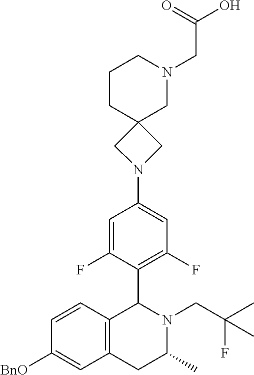 [SMILES: C[C@@H]1Cc2cc(OCc3ccccc3)ccc2C(c2c(F)cc(N3CC4(CCCN(CC(=O)O)C4)C3)cc2F)N1CC(C)(C)F] | 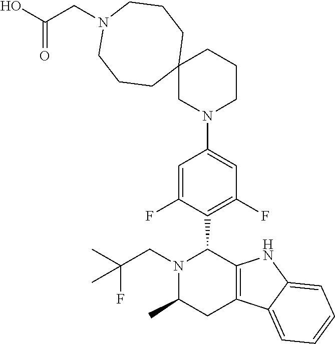 [SMILES: C[C@@H]1Cc2c([nH]c3ccccc23)[C@@H](c2c(F)cc(N3CCCC4(CCCN(CC(=O)O)CCC4)C3)cc2F)N1CC(C)(C)F] |
| 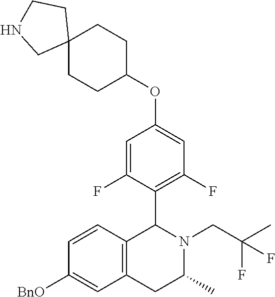 [SMILES: C[C@@H]1Cc2cc(OCc3ccccc3)ccc2C(c2c(F)cc(OC3CCC4(CCNC4)CC3)cc2F)N1CC(C)(F)F] | 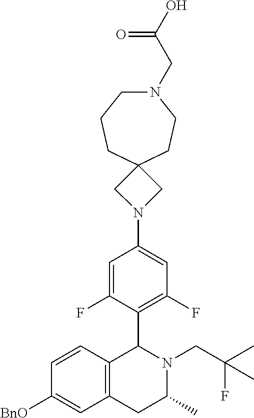 [SMILES: C[C@@H]1Cc2cc(OCc3ccccc3)ccc2C(c2c(F)cc(N3CC4(CCCN(CC(=O)O)CC4)C3)cc2F)N1CC(C)(C)F] | 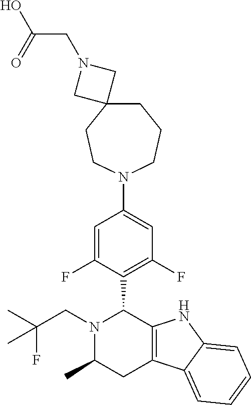 [SMILES: C[C@@H]1Cc2c([nH]c3ccccc23)[C@@H](c2c(F)cc(N3CCCC4(CC3)CN(CC(=O)O)C4)cc2F)N1CC(C)(C)F] |
| 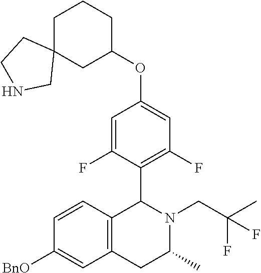 [SMILES: C[C@@H]1Cc2cc(OCc3ccccc3)ccc2C(c2c(F)cc(OC3CCCC4(CCNC4)C3)cc2F)N1CC(C)(F)F] | 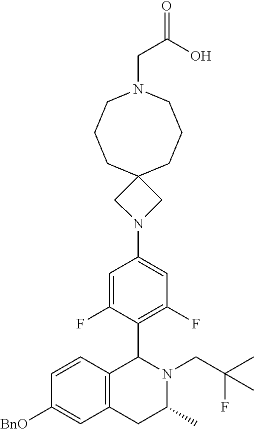 [SMILES: C[C@@H]1Cc2cc(OCc3ccccc3)ccc2C(c2c(F)cc(N3CC4(CCCN(CC(=O)O)CCC4)C3)cc2F)N1CC(C)(C)F] | 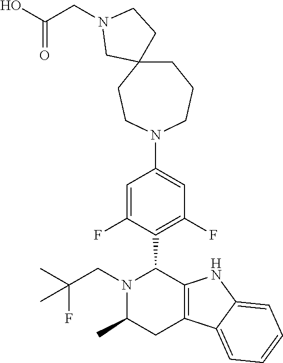 [SMILES: C[C@@H]1Cc2c([nH]c3ccccc23)[C@@H](c2c(F)cc(N3CCCC4(CCN(CC(=O)O)C4)CC3)cc2F)N1CC(C)(C)F] |
| 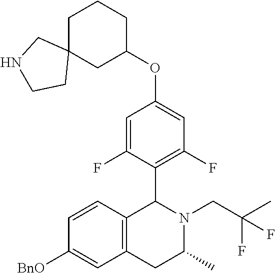 [SMILES: C[C@@H]1Cc2cc(OCc3ccccc3)ccc2C(c2c(F)cc(OC3CCCC4(CCNC4)C3)cc2F)N1CC(C)(F)F] | 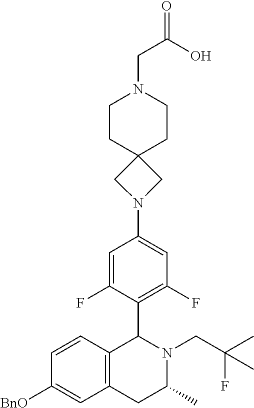 [SMILES: C[C@@H]1Cc2cc(OCc3ccccc3)ccc2C(c2c(F)cc(N3CC4(CCN(CC(=O)O)CC4)C3)cc2F)N1CC(C)(C)F] | 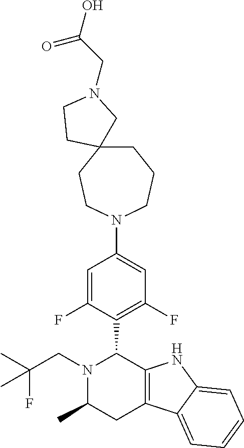 [SMILES: C[C@@H]1Cc2c([nH]c3ccccc23)[C@@H](c2c(F)cc(N3CCCC4(CCN(CC(=O)O)C4)CC3)cc2F)N1CC(C)(C)F] |
| 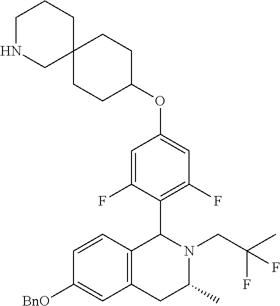 [SMILES: C[C@@H]1Cc2cc(OCc3ccccc3)ccc2C(c2c(F)cc(OC3CCC4(CCCNC4)CC3)cc2F)N1CC(C)(F)F] | 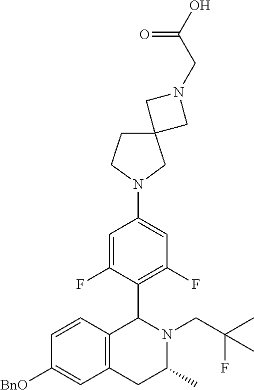 [SMILES: C[C@@H]1Cc2cc(OCc3ccccc3)ccc2C(c2c(F)cc(N3CCC4(CN(CC(=O)O)C4)C3)cc2F)N1CC(C)(C)F] | 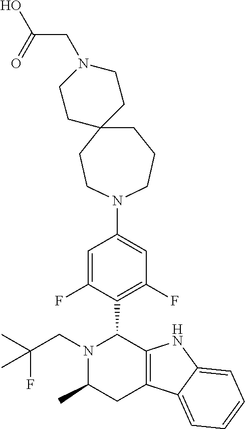 [SMILES: C[C@@H]1Cc2c([nH]c3ccccc23)[C@@H](c2c(F)cc(N3CCCC4(CCN(CC(=O)O)CC4)CC3)cc2F)N1CC(C)(C)F] |
| 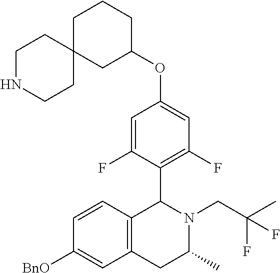 [SMILES: C[C@@H]1Cc2cc(OCc3ccccc3)ccc2C(c2c(F)cc(OC3CCCC4(CCNCC4)C3)cc2F)N1CC(C)(F)F] | 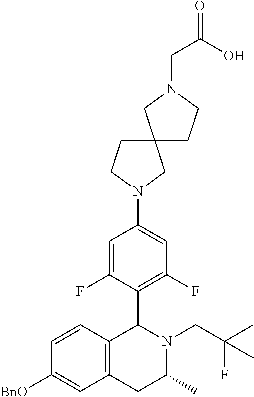 [SMILES: C[C@@H]1Cc2cc(OCc3ccccc3)ccc2C(c2c(F)cc(N3CCC4(CCN(CC(=O)O)C4)C3)cc2F)N1CC(C)(C)F] | 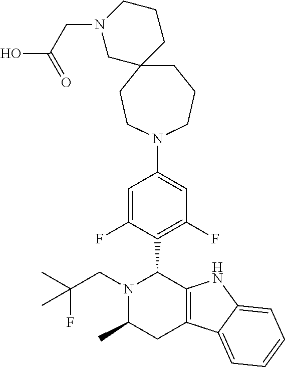 [SMILES: C[C@@H]1Cc2c([nH]c3ccccc23)[C@@H](c2c(F)cc(N3CCCC4(CCCN(CC(=O)O)C4)CC3)cc2F)N1CC(C)(C)F] |
| 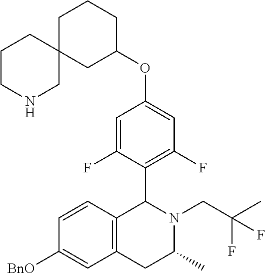 [SMILES: C[C@@H]1Cc2cc(OCc3ccccc3)ccc2C(c2c(F)cc(OC3CCCC4(CCCNC4)C3)cc2F)N1CC(C)(F)F] | 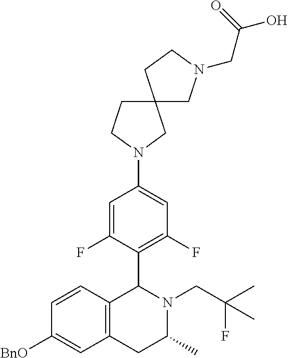 [SMILES: C[C@@H]1Cc2cc(OCc3ccccc3)ccc2C(c2c(F)cc(N3CCC4(CCN(CC(=O)O)C4)C3)cc2F)N1CC(C)(C)F] | 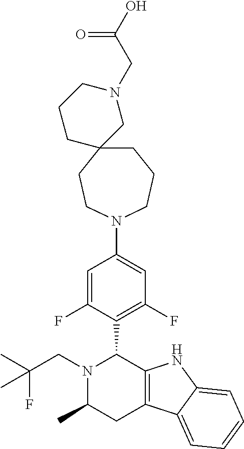 [SMILES: C[C@@H]1Cc2c([nH]c3ccccc23)[C@@H](c2c(F)cc(N3CCCC4(CCCN(CC(=O)O)C4)CC3)cc2F)N1CC(C)(C)F] |
| 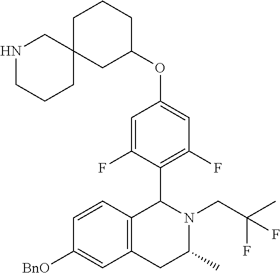 [SMILES: C[C@@H]1Cc2cc(OCc3ccccc3)ccc2C(c2c(F)cc(OC3CCCC4(CCCNC4)C3)cc2F)N1CC(C)(F)F] | 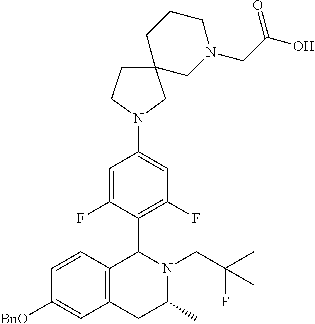 [SMILES: C[C@@H]1Cc2cc(OCc3ccccc3)ccc2C(c2c(F)cc(N3CCC4(CCCN(CC(=O)O)C4)C3)cc2F)N1CC(C)(C)F] | 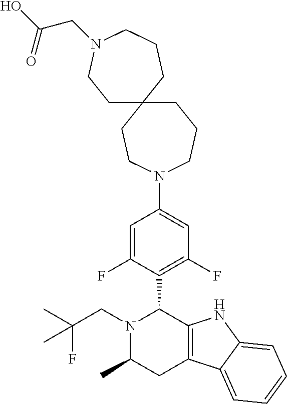 [SMILES: C[C@@H]1Cc2c([nH]c3ccccc23)[C@@H](c2c(F)cc(N3CCCC4(CCCN(CC(=O)O)CC4)CC3)cc2F)N1CC(C)(C)F] |
| 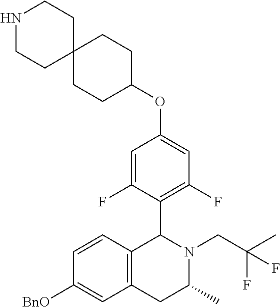 [SMILES: C[C@@H]1Cc2cc(OCc3ccccc3)ccc2C(c2c(F)cc(OC3CCC4(CCNCC4)CC3)cc2F)N1CC(C)(F)F] | 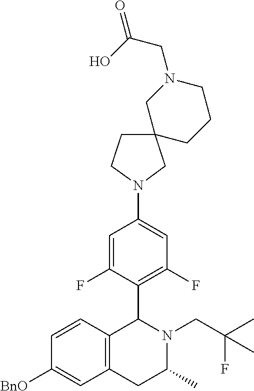 [SMILES: C[C@@H]1Cc2cc(OCc3ccccc3)ccc2C(c2c(F)cc(N3CCC4(CCCN(CC(=O)O)C4)C3)cc2F)N1CC(C)(C)F] | 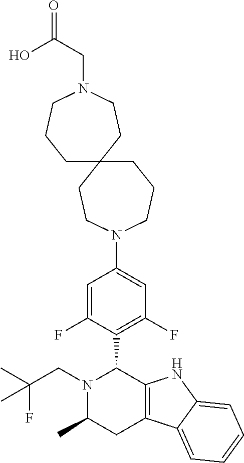 [SMILES: C[C@@H]1Cc2c([nH]c3ccccc23)[C@@H](c2c(F)cc(N3CCCC4(CCCN(CC(=O)O)CC4)CC3)cc2F)N1CC(C)(C)F] |
| 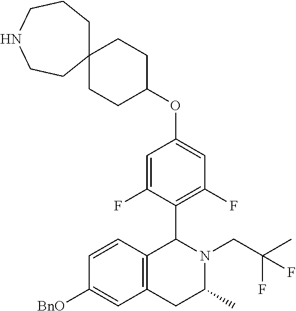 [SMILES: C[C@@H]1Cc2cc(OCc3ccccc3)ccc2C(c2c(F)cc(OC3CCC4(CCCNCC4)CC3)cc2F)N1CC(C)(F)F] | 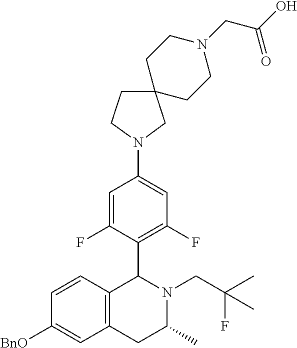 [SMILES: C[C@@H]1Cc2cc(OCc3ccccc3)ccc2C(c2c(F)cc(N3CCC4(CCN(CC(=O)O)CC4)C3)cc2F)N1CC(C)(C)F] | 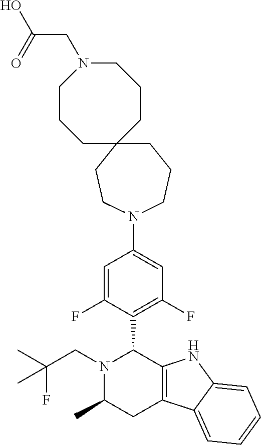 [SMILES: C[C@@H]1Cc2c([nH]c3ccccc23)[C@@H](c2c(F)cc(N3CCCC4(CCCN(CC(=O)O)CCC4)CC3)cc2F)N1CC(C)(C)F] |
| 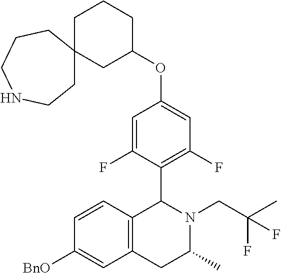 [SMILES: C[C@@H]1Cc2cc(OCc3ccccc3)ccc2C(c2c(F)cc(OC3CCCC4(CCCNCC4)C3)cc2F)N1CC(C)(F)F] | 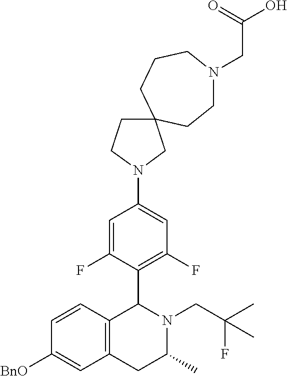 [SMILES: C[C@@H]1Cc2cc(OCc3ccccc3)ccc2C(c2c(F)cc(N3CCC4(CCCN(CC(=O)O)CC4)C3)cc2F)N1CC(C)(C)F] | 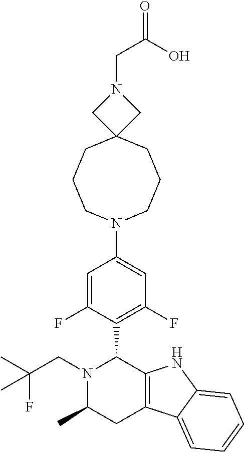 [SMILES: C[C@@H]1Cc2c([nH]c3ccccc23)[C@@H](c2c(F)cc(N3CCCC4(CCC3)CN(CC(=O)O)C4)cc2F)N1CC(C)(C)F] |
| 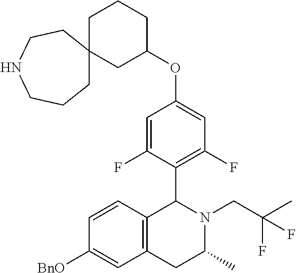 [SMILES: C[C@@H]1Cc2cc(OCc3ccccc3)ccc2C(c2c(F)cc(OC3CCCC4(CCCNCC4)C3)cc2F)N1CC(C)(F)F] | 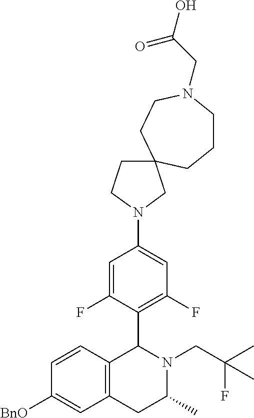 [SMILES: C[C@@H]1Cc2cc(OCc3ccccc3)ccc2C(c2c(F)cc(N3CCC4(CCCN(CC(=O)O)CC4)C3)cc2F)N1CC(C)(C)F] | 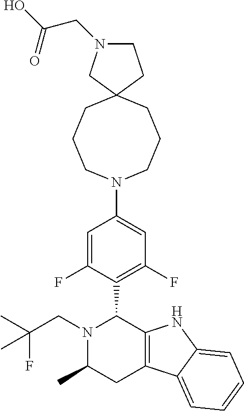 [SMILES: C[C@@H]1Cc2c([nH]c3ccccc23)[C@@H](c2c(F)cc(N3CCCC4(CCC3)CCN(CC(=O)O)C4)cc2F)N1CC(C)(C)F] |
| 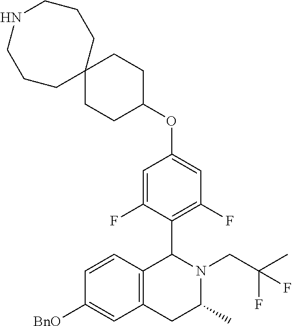 [SMILES: C[C@@H]1Cc2cc(OCc3ccccc3)ccc2C(c2c(F)cc(OC3CCC4(CCCNCCC4)CC3)cc2F)N1CC(C)(F)F] | 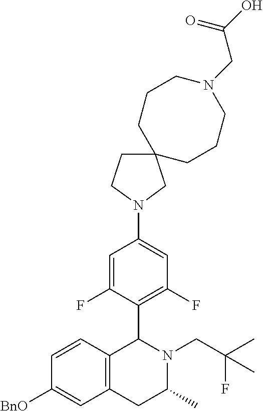 [SMILES: C[C@@H]1Cc2cc(OCc3ccccc3)ccc2C(c2c(F)cc(N3CCC4(CCCN(CC(=O)O)CCC4)C3)cc2F)N1CC(C)(C)F] | 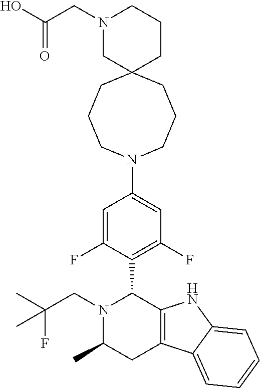 [SMILES: C[C@@H]1Cc2c([nH]c3ccccc23)[C@@H](c2c(F)cc(N3CCCC4(CCCN(CC(=O)O)C4)CCC3)cc2F)N1CC(C)(C)F] |
| 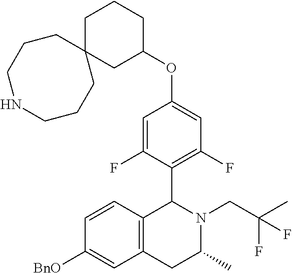 [SMILES: C[C@@H]1Cc2cc(OCc3ccccc3)ccc2C(c2c(F)cc(OC3CCCC4(CCCNCCC4)C3)cc2F)N1CC(C)(F)F] | 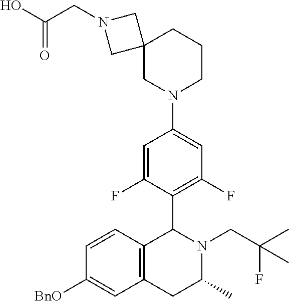 [SMILES: C[C@@H]1Cc2cc(OCc3ccccc3)ccc2C(c2c(F)cc(N3CCCC4(CN(CC(=O)O)C4)C3)cc2F)N1CC(C)(C)F] | 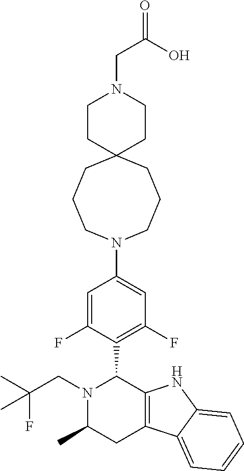 [SMILES: C[C@@H]1Cc2c([nH]c3ccccc23)[C@@H](c2c(F)cc(N3CCCC4(CCC3)CCN(CC(=O)O)CC4)cc2F)N1CC(C)(C)F] |
| 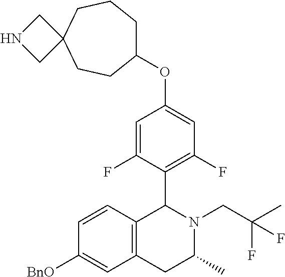 [SMILES: C[C@@H]1Cc2cc(OCc3ccccc3)ccc2C(c2c(F)cc(OC3CCCC4(CC3)CNC4)cc2F)N1CC(C)(F)F] | 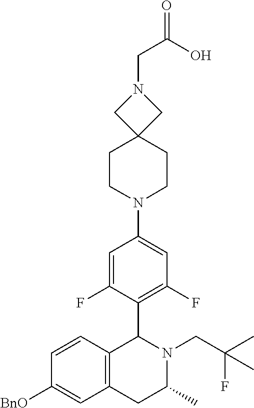 [SMILES: C[C@@H]1Cc2cc(OCc3ccccc3)ccc2C(c2c(F)cc(N3CCC4(CC3)CN(CC(=O)O)C4)cc2F)N1CC(C)(C)F] | 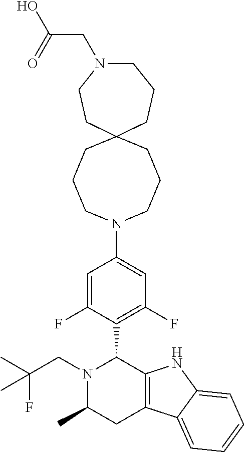 [SMILES: C[C@@H]1Cc2c([nH]c3ccccc23)[C@@H](c2c(F)cc(N3CCCC4(CCCN(CC(=O)O)CC4)CCC3)cc2F)N1CC(C)(C)F] |
| 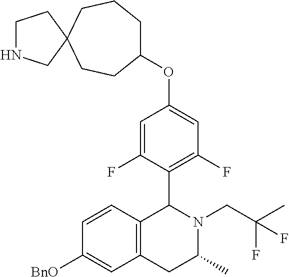 [SMILES: C[C@@H]1Cc2cc(OCc3ccccc3)ccc2C(c2c(F)cc(OC3CCCC4(CCNC4)CC3)cc2F)N1CC(C)(F)F] | 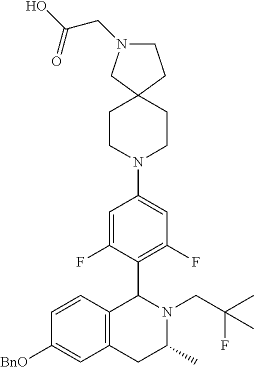 [SMILES: C[C@@H]1Cc2cc(OCc3ccccc3)ccc2C(c2c(F)cc(N3CCC4(CCN(CC(=O)O)C4)CC3)cc2F)N1CC(C)(C)F] | 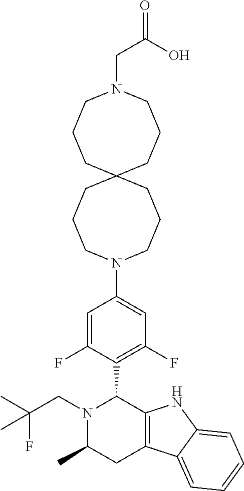 [SMILES: C[C@@H]1Cc2c([nH]c3ccccc23)[C@@H](c2c(F)cc(N3CCCC4(CCCN(CC(=O)O)CCC4)CCC3)cc2F)N1CC(C)(C)F] |
| 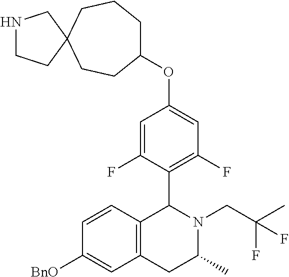 [SMILES: C[C@@H]1Cc2cc(OCc3ccccc3)ccc2C(c2c(F)cc(OC3CCCC4(CCNC4)CC3)cc2F)N1CC(C)(F)F] | 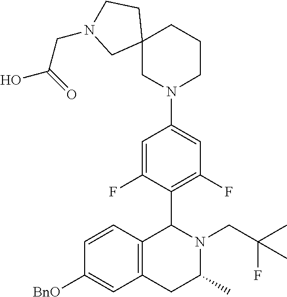 [SMILES: C[C@@H]1Cc2cc(OCc3ccccc3)ccc2C(c2c(F)cc(N3CCCC4(CCN(CC(=O)O)C4)C3)cc2F)N1CC(C)(C)F] | 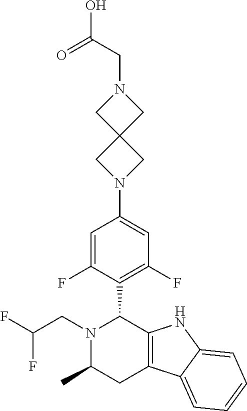 [SMILES: C[C@@H]1Cc2c([nH]c3ccccc23)[C@@H](c2c(F)cc(N3CC4(CN(CC(=O)O)C4)C3)cc2F)N1CC(F)F] |
| 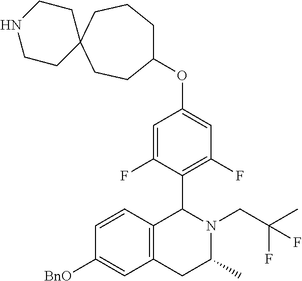 [SMILES: C[C@@H]1Cc2cc(OCc3ccccc3)ccc2C(c2c(F)cc(OC3CCCC4(CCNCC4)CC3)cc2F)N1CC(C)(F)F] | 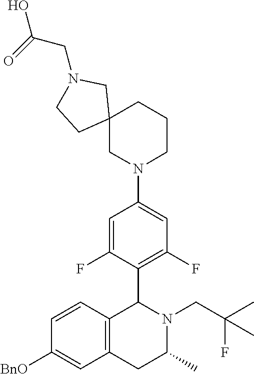 [SMILES: C[C@@H]1Cc2cc(OCc3ccccc3)ccc2C(c2c(F)cc(N3CCCC4(CCN(CC(=O)O)C4)C3)cc2F)N1CC(C)(C)F] | 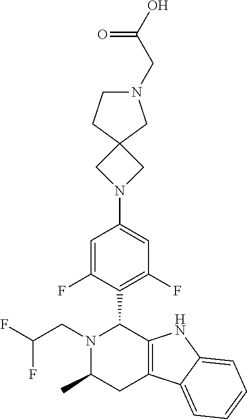 [SMILES: C[C@@H]1Cc2c([nH]c3ccccc23)[C@@H](c2c(F)cc(N3CC4(CCN(CC(=O)O)C4)C3)cc2F)N1CC(F)F] |
| 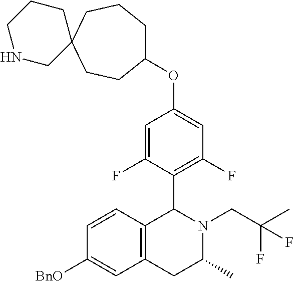 [SMILES: C[C@@H]1Cc2cc(OCc3ccccc3)ccc2C(c2c(F)cc(OC3CCCC4(CCCNC4)CC3)cc2F)N1CC(C)(F)F] | 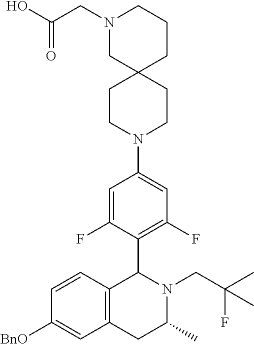 [SMILES: C[C@@H]1Cc2cc(OCc3ccccc3)ccc2C(c2c(F)cc(N3CCC4(CCCN(CC(=O)O)C4)CC3)cc2F)N1CC(C)(C)F] | 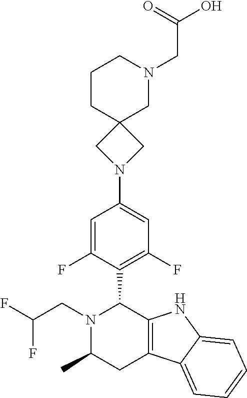 [SMILES: C[C@@H]1Cc2c([nH]c3ccccc23)[C@@H](c2c(F)cc(N3CC4(CCCN(CC(=O)O)C4)C3)cc2F)N1CC(F)F] |
| 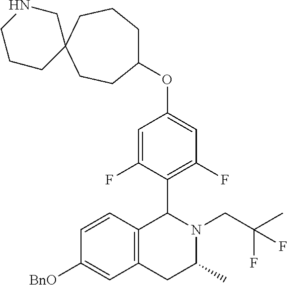 [SMILES: C[C@@H]1Cc2cc(OCc3ccccc3)ccc2C(c2c(F)cc(OC3CCCC4(CCCNC4)CC3)cc2F)N1CC(C)(F)F] | 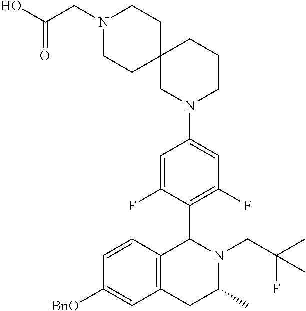 [SMILES: C[C@@H]1Cc2cc(OCc3ccccc3)ccc2C(c2c(F)cc(N3CCCC4(CCN(CC(=O)O)CC4)C3)cc2F)N1CC(C)(C)F] | 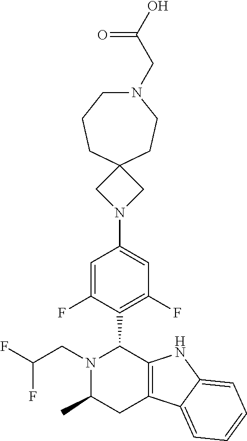 [SMILES: C[C@@H]1Cc2c([nH]c3ccccc23)[C@@H](c2c(F)cc(N3CC4(CCCN(CC(=O)O)CC4)C3)cc2F)N1CC(F)F] |
| 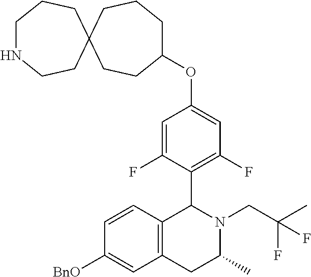 [SMILES: C[C@@H]1Cc2cc(OCc3ccccc3)ccc2C(c2c(F)cc(OC3CCCC4(CCCNCC4)CC3)cc2F)N1CC(C)(F)F] | 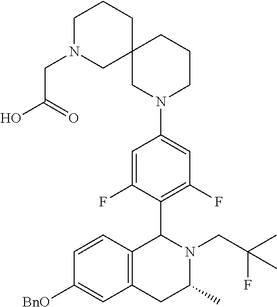 [SMILES: C[C@@H]1Cc2cc(OCc3ccccc3)ccc2C(c2c(F)cc(N3CCCC4(CCCN(CC(=O)O)C4)C3)cc2F)N1CC(C)(C)F] | 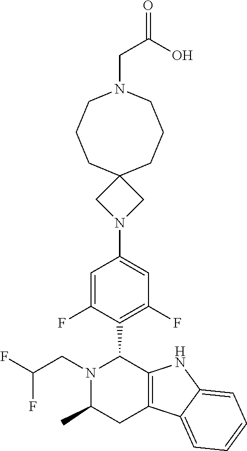 [SMILES: C[C@@H]1Cc2c([nH]c3ccccc23)[C@@H](c2c(F)cc(N3CC4(CCCN(CC(=O)O)CCC4)C3)cc2F)N1CC(F)F] |
| 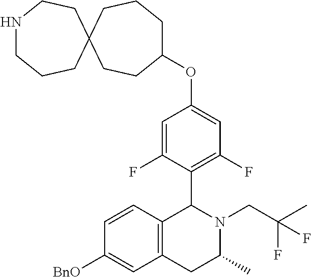 [SMILES: C[C@@H]1Cc2cc(OCc3ccccc3)ccc2C(c2c(F)cc(OC3CCCC4(CCCNCC4)CC3)cc2F)N1CC(C)(F)F] | 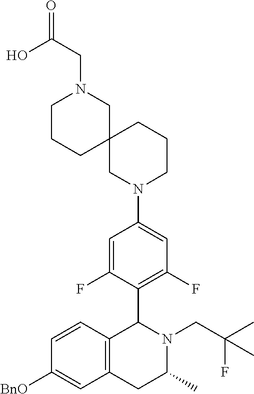 [SMILES: C[C@@H]1Cc2cc(OCc3ccccc3)ccc2C(c2c(F)cc(N3CCCC4(CCCN(CC(=O)O)C4)C3)cc2F)N1CC(C)(C)F] | 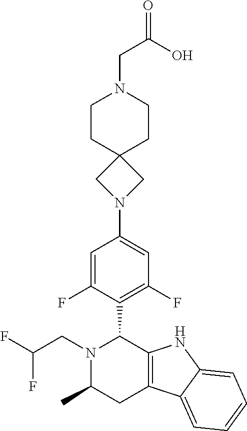 [SMILES: C[C@@H]1Cc2c([nH]c3ccccc23)[C@@H](c2c(F)cc(N3CC4(CCN(CC(=O)O)CC4)C3)cc2F)N1CC(F)F] |
| 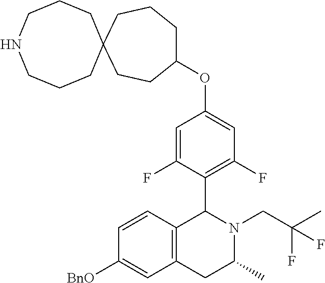 [SMILES: C[C@@H]1Cc2cc(OCc3ccccc3)ccc2C(c2c(F)cc(OC3CCCC4(CCCNCCC4)CC3)cc2F)N1CC(C)(F)F] | 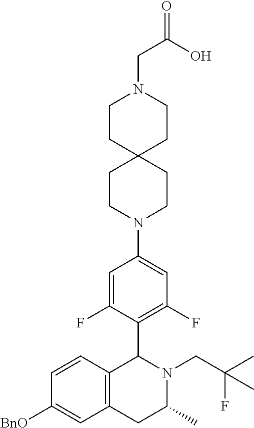 [SMILES: C[C@@H]1Cc2cc(OCc3ccccc3)ccc2C(c2c(F)cc(N3CCC4(CCN(CC(=O)O)CC4)CC3)cc2F)N1CC(C)(C)F] | 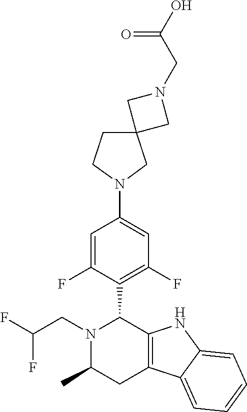 [SMILES: C[C@@H]1Cc2c([nH]c3ccccc23)[C@@H](c2c(F)cc(N3CCC4(CN(CC(=O)O)C4)C3)cc2F)N1CC(F)F] |
| 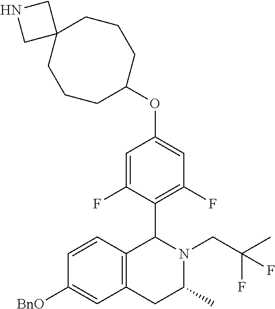 [SMILES: C[C@@H]1Cc2cc(OCc3ccccc3)ccc2C(c2c(F)cc(OC3CCCC4(CCC3)CNC4)cc2F)N1CC(C)(F)F] | 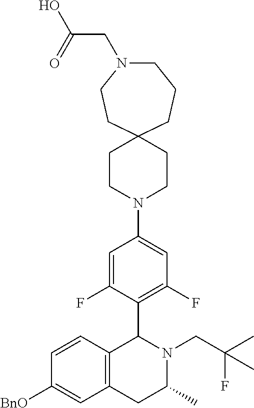 [SMILES: C[C@@H]1Cc2cc(OCc3ccccc3)ccc2C(c2c(F)cc(N3CCC4(CCCN(CC(=O)O)CC4)CC3)cc2F)N1CC(C)(C)F] | 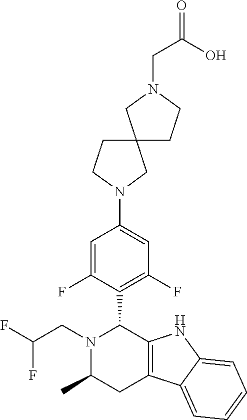 [SMILES: C[C@@H]1Cc2c([nH]c3ccccc23)[C@@H](c2c(F)cc(N3CCC4(CCN(CC(=O)O)C4)C3)cc2F)N1CC(F)F] |
| 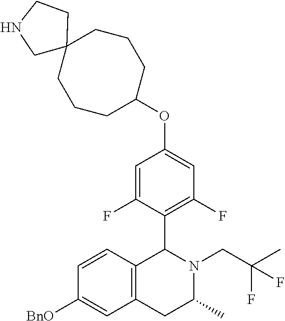 [SMILES: C[C@@H]1Cc2cc(OCc3ccccc3)ccc2C(c2c(F)cc(OC3CCCC4(CCC3)CCNC4)cc2F)N1CC(C)(F)F] | 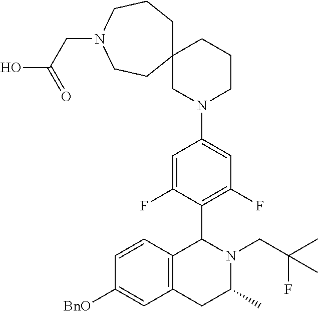 [SMILES: C[C@@H]1Cc2cc(OCc3ccccc3)ccc2C(c2c(F)cc(N3CCCC4(CCCN(CC(=O)O)CC4)C3)cc2F)N1CC(C)(C)F] | 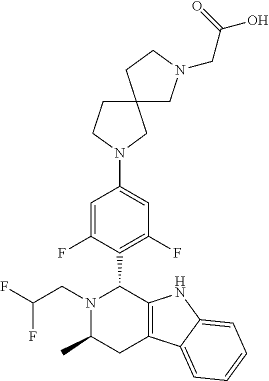 [SMILES: C[C@@H]1Cc2c([nH]c3ccccc23)[C@@H](c2c(F)cc(N3CCC4(CCN(CC(=O)O)C4)C3)cc2F)N1CC(F)F] |
| 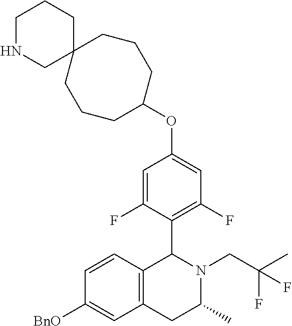 [SMILES: C[C@@H]1Cc2cc(OCc3ccccc3)ccc2C(c2c(F)cc(OC3CCCC4(CCCNC4)CCC3)cc2F)N1CC(C)(F)F] | 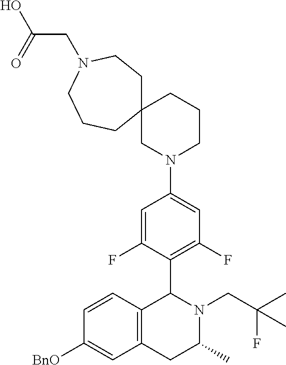 [SMILES: C[C@@H]1Cc2cc(OCc3ccccc3)ccc2C(c2c(F)cc(N3CCCC4(CCCN(CC(=O)O)CC4)C3)cc2F)N1CC(C)(C)F] | 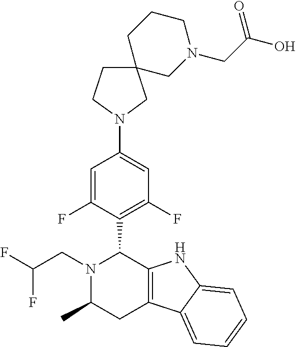 [SMILES: C[C@@H]1Cc2c([nH]c3ccccc23)[C@@H](c2c(F)cc(N3CCC4(CCCN(CC(=O)O)C4)C3)cc2F)N1CC(F)F] |
| 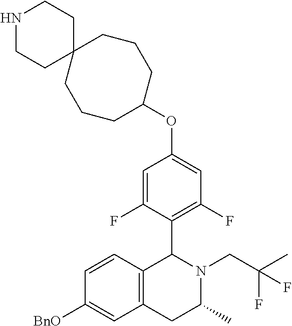 [SMILES: C[C@@H]1Cc2cc(OCc3ccccc3)ccc2C(c2c(F)cc(OC3CCCC4(CCC3)CCNCC4)cc2F)N1CC(C)(F)F] | 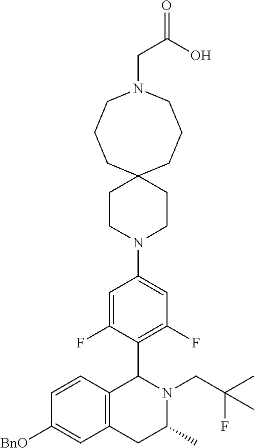 [SMILES: C[C@@H]1Cc2cc(OCc3ccccc3)ccc2C(c2c(F)cc(N3CCC4(CCCN(CC(=O)O)CCC4)CC3)cc2F)N1CC(C)(C)F] | 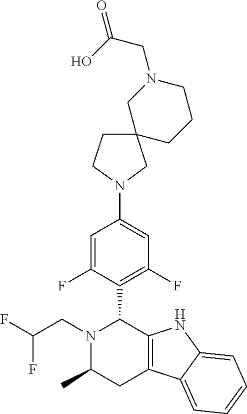 [SMILES: C[C@@H]1Cc2c([nH]c3ccccc23)[C@@H](c2c(F)cc(N3CCC4(CCCN(CC(=O)O)C4)C3)cc2F)N1CC(F)F] |
| 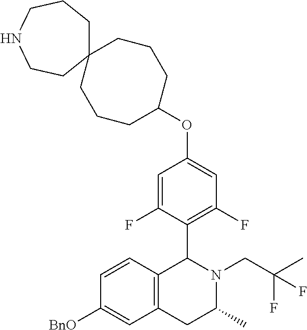 [SMILES: C[C@@H]1Cc2cc(OCc3ccccc3)ccc2C(c2c(F)cc(OC3CCCC4(CCCNCC4)CCC3)cc2F)N1CC(C)(F)F] | 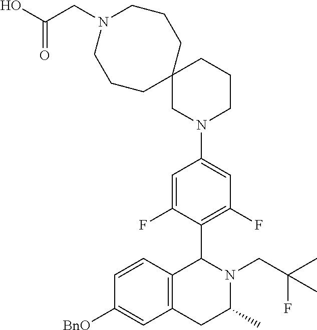 [SMILES: C[C@@H]1Cc2cc(OCc3ccccc3)ccc2C(c2c(F)cc(N3CCCC4(CCCN(CC(=O)O)CCC4)C3)cc2F)N1CC(C)(C)F] | 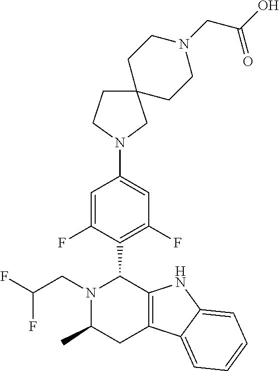 [SMILES: C[C@@H]1Cc2c([nH]c3ccccc23)[C@@H](c2c(F)cc(N3CCC4(CCN(CC(=O)O)CC4)C3)cc2F)N1CC(F)F] |
| 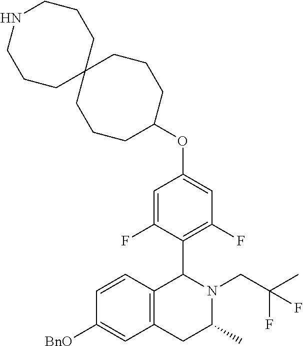 [SMILES: C[C@@H]1Cc2cc(OCc3ccccc3)ccc2C(c2c(F)cc(OC3CCCC4(CCCNCCC4)CCC3)cc2F)N1CC(C)(F)F] | 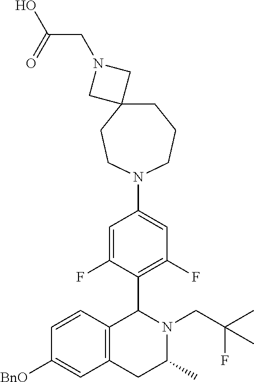 [SMILES: C[C@@H]1Cc2cc(OCc3ccccc3)ccc2C(c2c(F)cc(N3CCCC4(CC3)CN(CC(=O)O)C4)cc2F)N1CC(C)(C)F] | 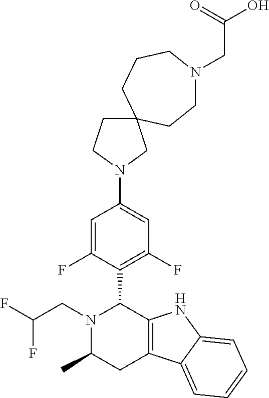 [SMILES: C[C@@H]1Cc2c([nH]c3ccccc23)[C@@H](c2c(F)cc(N3CCC4(CCCN(CC(=O)O)CC4)C3)cc2F)N1CC(F)F] |
| 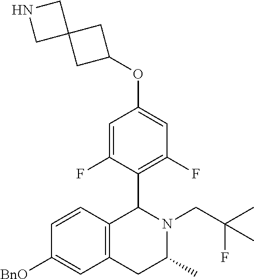 [SMILES: C[C@@H]1Cc2cc(OCc3ccccc3)ccc2C(c2c(F)cc(OC3CC4(CNC4)C3)cc2F)N1CC(C)(C)F] | 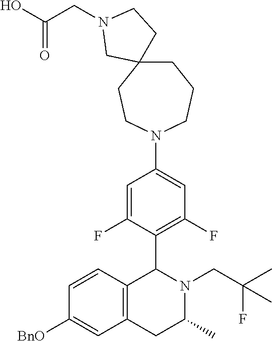 [SMILES: C[C@@H]1Cc2cc(OCc3ccccc3)ccc2C(c2c(F)cc(N3CCCC4(CCN(CC(=O)O)C4)CC3)cc2F)N1CC(C)(C)F] | 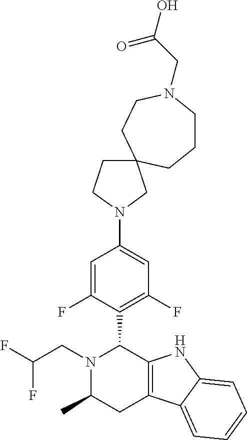 [SMILES: C[C@@H]1Cc2c([nH]c3ccccc23)[C@@H](c2c(F)cc(N3CCC4(CCCN(CC(=O)O)CC4)C3)cc2F)N1CC(F)F] |
| 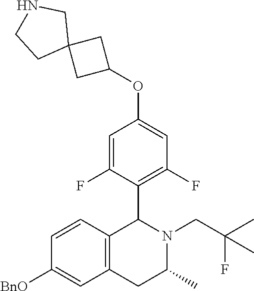 [SMILES: C[C@@H]1Cc2cc(OCc3ccccc3)ccc2C(c2c(F)cc(OC3CC4(CCNC4)C3)cc2F)N1CC(C)(C)F] | 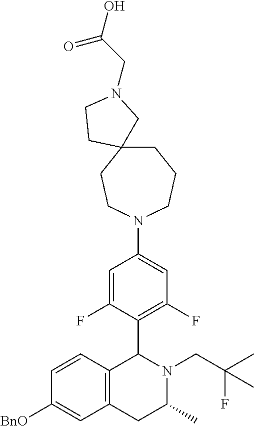 [SMILES: C[C@@H]1Cc2cc(OCc3ccccc3)ccc2C(c2c(F)cc(N3CCCC4(CCN(CC(=O)O)C4)CC3)cc2F)N1CC(C)(C)F] | 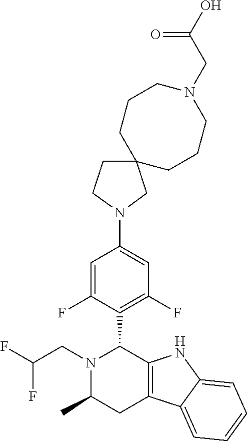 [SMILES: C[C@@H]1Cc2c([nH]c3ccccc23)[C@@H](c2c(F)cc(N3CCC4(CCCN(CC(=O)O)CCC4)C3)cc2F)N1CC(F)F] |
| 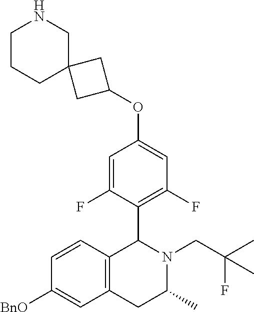 [SMILES: C[C@@H]1Cc2cc(OCc3ccccc3)ccc2C(c2c(F)cc(OC3CC4(CCCNC4)C3)cc2F)N1CC(C)(C)F] | 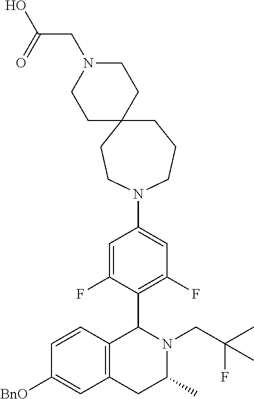 [SMILES: C[C@@H]1Cc2cc(OCc3ccccc3)ccc2C(c2c(F)cc(N3CCCC4(CCN(CC(=O)O)CC4)CC3)cc2F)N1CC(C)(C)F] | 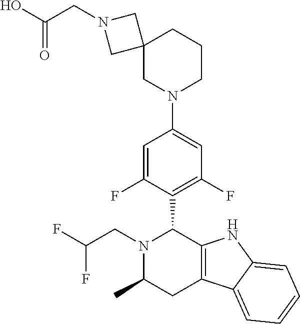 [SMILES: C[C@@H]1Cc2c([nH]c3ccccc23)[C@@H](c2c(F)cc(N3CCCC4(CN(CC(=O)O)C4)C3)cc2F)N1CC(F)F] |
| 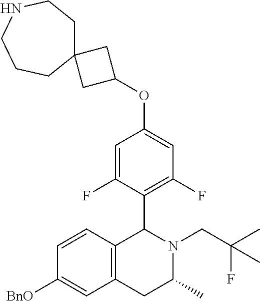 [SMILES: C[C@@H]1Cc2cc(OCc3ccccc3)ccc2C(c2c(F)cc(OC3CC4(CCCNCC4)C3)cc2F)N1CC(C)(C)F] | 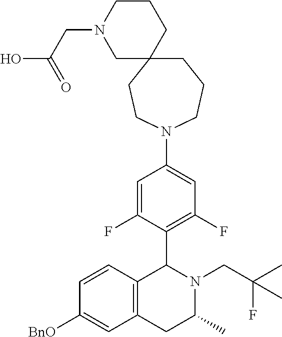 [SMILES: C[C@@H]1Cc2cc(OCc3ccccc3)ccc2C(c2c(F)cc(N3CCCC4(CCCN(CC(=O)O)C4)CC3)cc2F)N1CC(C)(C)F] | 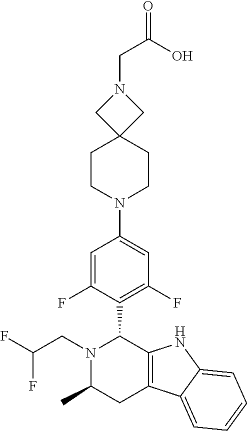 [SMILES: C[C@@H]1Cc2c([nH]c3ccccc23)[C@@H](c2c(F)cc(N3CCC4(CC3)CN(CC(=O)O)C4)cc2F)N1CC(F)F] |
| 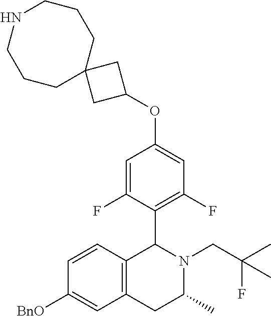 [SMILES: C[C@@H]1Cc2cc(OCc3ccccc3)ccc2C(c2c(F)cc(OC3CC4(CCCNCCC4)C3)cc2F)N1CC(C)(C)F] | 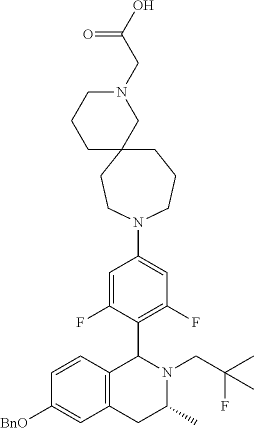 [SMILES: C[C@@H]1Cc2cc(OCc3ccccc3)ccc2C(c2c(F)cc(N3CCCC4(CCCN(CC(=O)O)C4)CC3)cc2F)N1CC(C)(C)F] | 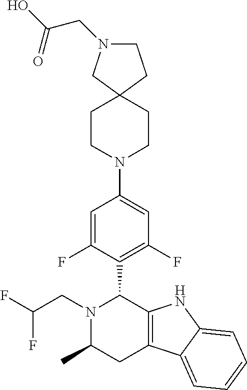 [SMILES: C[C@@H]1Cc2c([nH]c3ccccc23)[C@@H](c2c(F)cc(N3CCC4(CCN(CC(=O)O)C4)CC3)cc2F)N1CC(F)F] |
| 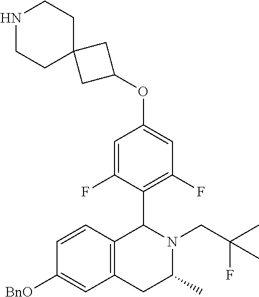 [SMILES: C[C@@H]1Cc2cc(OCc3ccccc3)ccc2C(c2c(F)cc(OC3CC4(CCNCC4)C3)cc2F)N1CC(C)(C)F] | 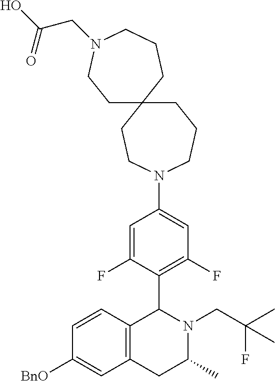 [SMILES: C[C@@H]1Cc2cc(OCc3ccccc3)ccc2C(c2c(F)cc(N3CCCC4(CCCN(CC(=O)O)CC4)CC3)cc2F)N1CC(C)(C)F] | 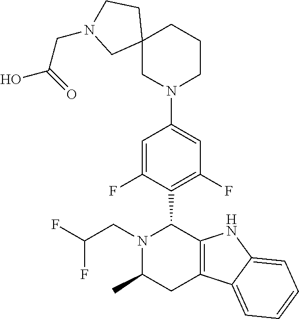 [SMILES: C[C@@H]1Cc2c([nH]c3ccccc23)[C@@H](c2c(F)cc(N3CCCC4(CCN(CC(=O)O)C4)C3)cc2F)N1CC(F)F] |
| 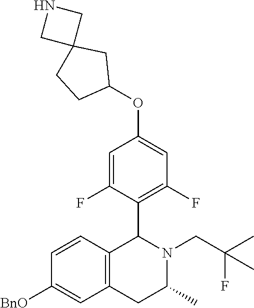 [SMILES: C[C@@H]1Cc2cc(OCc3ccccc3)ccc2C(c2c(F)cc(OC3CCC4(CNC4)C3)cc2F)N1CC(C)(C)F] | 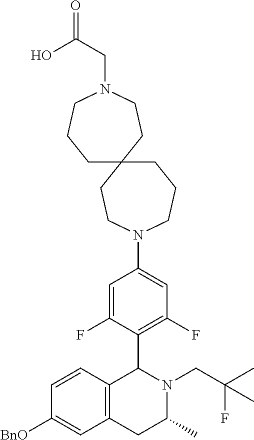 [SMILES: C[C@@H]1Cc2cc(OCc3ccccc3)ccc2C(c2c(F)cc(N3CCCC4(CCCN(CC(=O)O)CC4)CC3)cc2F)N1CC(C)(C)F] | 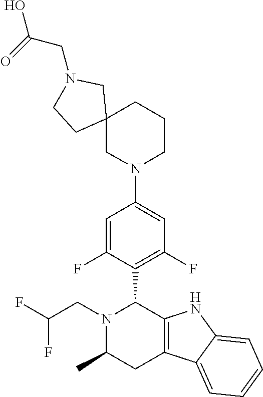 [SMILES: C[C@@H]1Cc2c([nH]c3ccccc23)[C@@H](c2c(F)cc(N3CCCC4(CCN(CC(=O)O)C4)C3)cc2F)N1CC(F)F] |
| 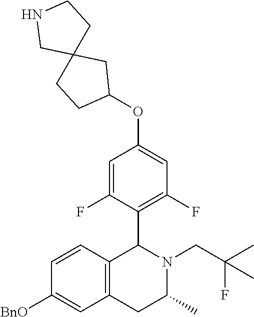 [SMILES: C[C@@H]1Cc2cc(OCc3ccccc3)ccc2C(c2c(F)cc(OC3CCC4(CCNC4)C3)cc2F)N1CC(C)(C)F] | 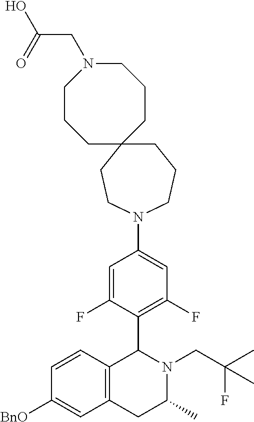 [SMILES: C[C@@H]1Cc2cc(OCc3ccccc3)ccc2C(c2c(F)cc(N3CCCC4(CCCN(CC(=O)O)CCC4)CC3)cc2F)N1CC(C)(C)F] | 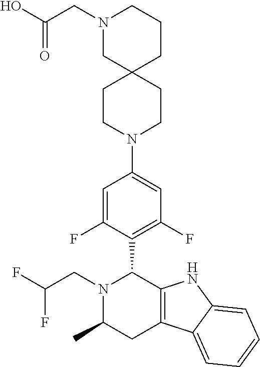 [SMILES: C[C@@H]1Cc2c([nH]c3ccccc23)[C@@H](c2c(F)cc(N3CCC4(CCCN(CC(=O)O)C4)CC3)cc2F)N1CC(F)F] |
| 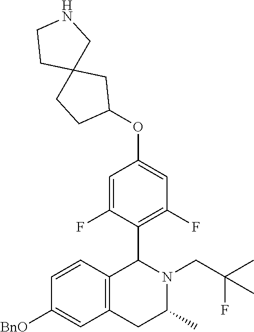 [SMILES: C[C@@H]1Cc2cc(OCc3ccccc3)ccc2C(c2c(F)cc(OC3CCC4(CCNC4)C3)cc2F)N1CC(C)(C)F] | 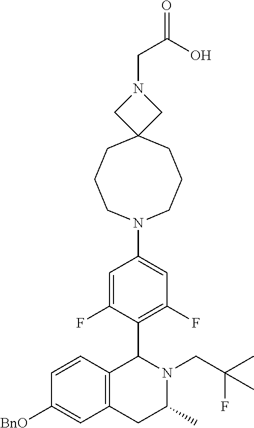 [SMILES: C[C@@H]1Cc2cc(OCc3ccccc3)ccc2C(c2c(F)cc(N3CCCC4(CCC3)CN(CC(=O)O)C4)cc2F)N1CC(C)(C)F] | 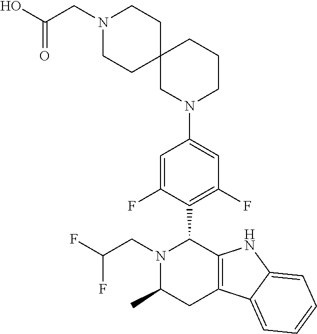 [SMILES: C[C@@H]1Cc2c([nH]c3ccccc23)[C@@H](c2c(F)cc(N3CCCC4(CCN(CC(=O)O)CC4)C3)cc2F)N1CC(F)F] |
| 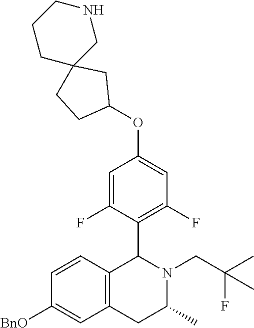 [SMILES: C[C@@H]1Cc2cc(OCc3ccccc3)ccc2C(c2c(F)cc(OC3CCC4(CCCNC4)C3)cc2F)N1CC(C)(C)F] | 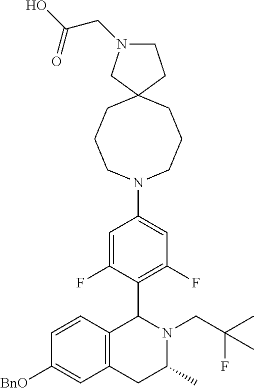 [SMILES: C[C@@H]1Cc2cc(OCc3ccccc3)ccc2C(c2c(F)cc(N3CCCC4(CCC3)CCN(CC(=O)O)C4)cc2F)N1CC(C)(C)F] | 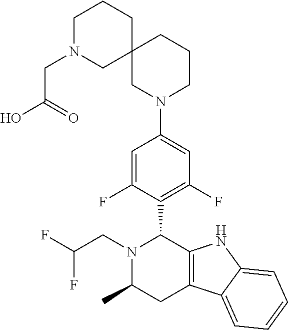 [SMILES: C[C@@H]1Cc2c([nH]c3ccccc23)[C@@H](c2c(F)cc(N3CCCC4(CCCN(CC(=O)O)C4)C3)cc2F)N1CC(F)F] |
| 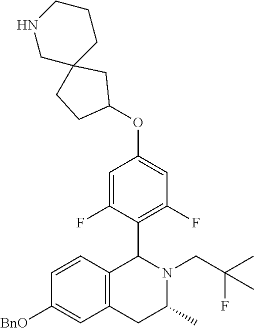 [SMILES: C[C@@H]1Cc2cc(OCc3ccccc3)ccc2C(c2c(F)cc(OC3CCC4(CCCNC4)C3)cc2F)N1CC(C)(C)F] | 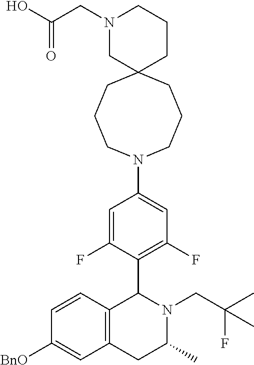 [SMILES: C[C@@H]1Cc2cc(OCc3ccccc3)ccc2C(c2c(F)cc(N3CCCC4(CCCN(CC(=O)O)C4)CCC3)cc2F)N1CC(C)(C)F] | 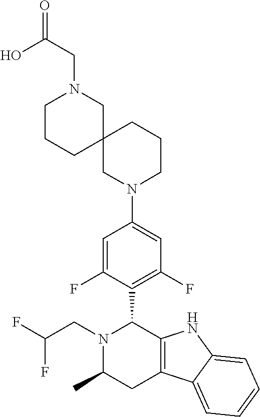 [SMILES: C[C@@H]1Cc2c([nH]c3ccccc23)[C@@H](c2c(F)cc(N3CCCC4(CCCN(CC(=O)O)C4)C3)cc2F)N1CC(F)F] |
| 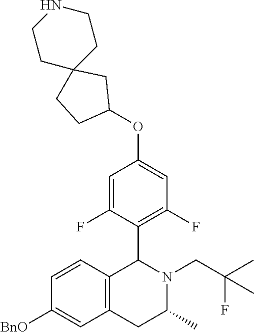 [SMILES: C[C@@H]1Cc2cc(OCc3ccccc3)ccc2C(c2c(F)cc(OC3CCC4(CCNCC4)C3)cc2F)N1CC(C)(C)F] | 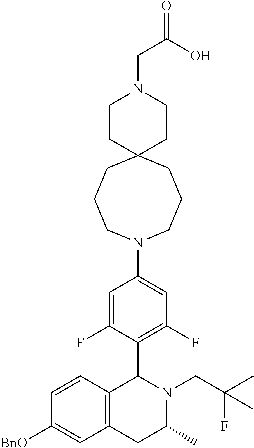 [SMILES: C[C@@H]1Cc2cc(OCc3ccccc3)ccc2C(c2c(F)cc(N3CCCC4(CCC3)CCN(CC(=O)O)CC4)cc2F)N1CC(C)(C)F] | 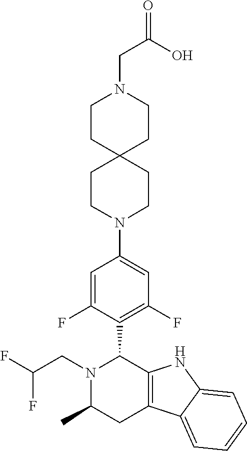 [SMILES: C[C@@H]1Cc2c([nH]c3ccccc23)[C@@H](c2c(F)cc(N3CCC4(CCN(CC(=O)O)CC4)CC3)cc2F)N1CC(F)F] |
| 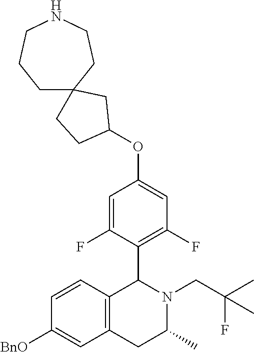 [SMILES: C[C@@H]1Cc2cc(OCc3ccccc3)ccc2C(c2c(F)cc(OC3CCC4(CCCNCC4)C3)cc2F)N1CC(C)(C)F] | 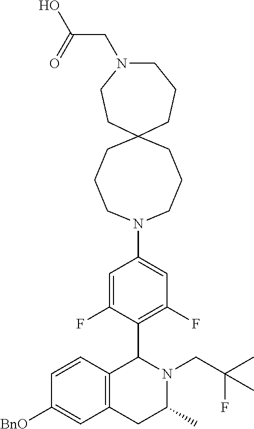 [SMILES: C[C@@H]1Cc2cc(OCc3ccccc3)ccc2C(c2c(F)cc(N3CCCC4(CCCN(CC(=O)O)CC4)CCC3)cc2F)N1CC(C)(C)F] | 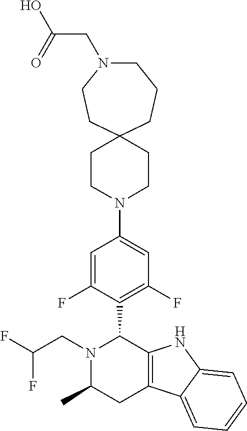 [SMILES: C[C@@H]1Cc2c([nH]c3ccccc23)[C@@H](c2c(F)cc(N3CCC4(CCCN(CC(=O)O)CC4)CC3)cc2F)N1CC(F)F] |
| 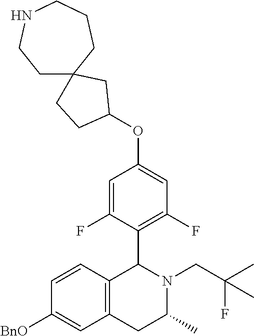 [SMILES: C[C@@H]1Cc2cc(OCc3ccccc3)ccc2C(c2c(F)cc(OC3CCC4(CCCNCC4)C3)cc2F)N1CC(C)(C)F] | 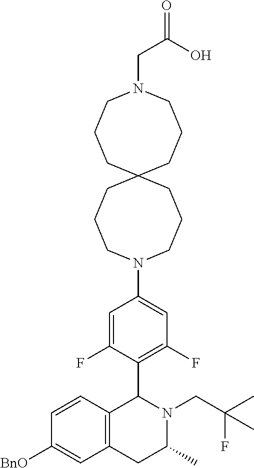 [SMILES: C[C@@H]1Cc2cc(OCc3ccccc3)ccc2C(c2c(F)cc(N3CCCC4(CCCN(CC(=O)O)CCC4)CCC3)cc2F)N1CC(C)(C)F] | 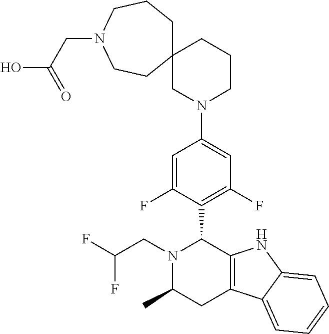 [SMILES: C[C@@H]1Cc2c([nH]c3ccccc23)[C@@H](c2c(F)cc(N3CCCC4(CCCN(CC(=O)O)CC4)C3)cc2F)N1CC(F)F] |
| 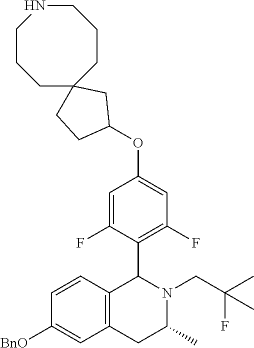 [SMILES: C[C@@H]1Cc2cc(OCc3ccccc3)ccc2C(c2c(F)cc(OC3CCC4(CCCNCCC4)C3)cc2F)N1CC(C)(C)F] | 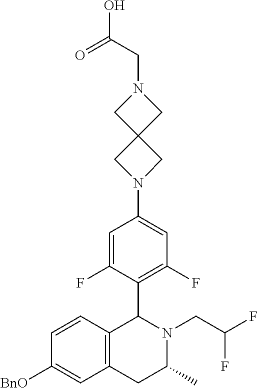 [SMILES: C[C@@H]1Cc2cc(OCc3ccccc3)ccc2C(c2c(F)cc(N3CC4(CN(CC(=O)O)C4)C3)cc2F)N1CC(F)F] | 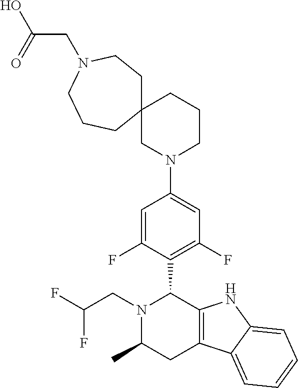 [SMILES: C[C@@H]1Cc2c([nH]c3ccccc23)[C@@H](c2c(F)cc(N3CCCC4(CCCN(CC(=O)O)CC4)C3)cc2F)N1CC(F)F] |
| 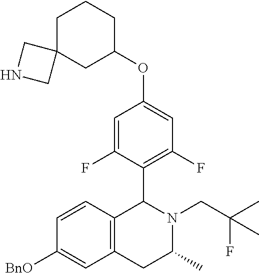 [SMILES: C[C@@H]1Cc2cc(OCc3ccccc3)ccc2C(c2c(F)cc(OC3CCCC4(CNC4)C3)cc2F)N1CC(C)(C)F] | 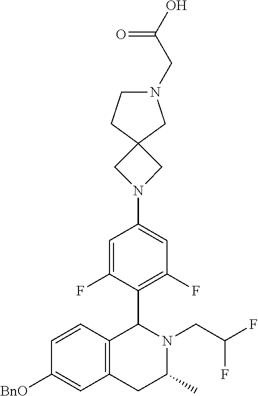 [SMILES: C[C@@H]1Cc2cc(OCc3ccccc3)ccc2C(c2c(F)cc(N3CC4(CCN(CC(=O)O)C4)C3)cc2F)N1CC(F)F] | 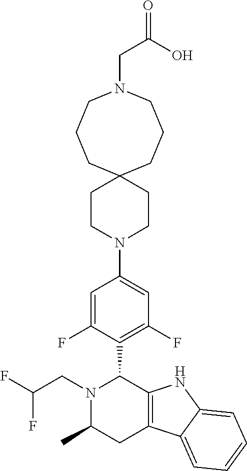 [SMILES: C[C@@H]1Cc2c([nH]c3ccccc23)[C@@H](c2c(F)cc(N3CCC4(CCCN(CC(=O)O)CCC4)CC3)cc2F)N1CC(F)F] |
| 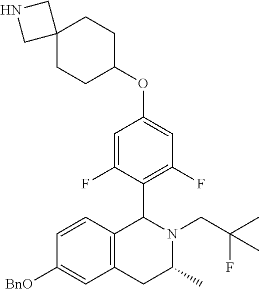 [SMILES: C[C@@H]1Cc2cc(OCc3ccccc3)ccc2C(c2c(F)cc(OC3CCC4(CC3)CNC4)cc2F)N1CC(C)(C)F] | 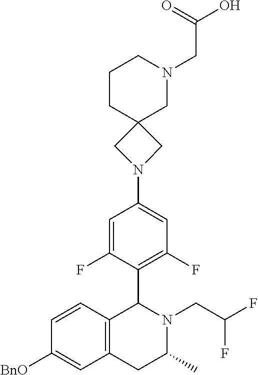 [SMILES: C[C@@H]1Cc2cc(OCc3ccccc3)ccc2C(c2c(F)cc(N3CC4(CCCN(CC(=O)O)C4)C3)cc2F)N1CC(F)F] | 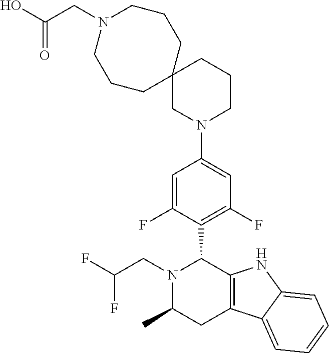 [SMILES: C[C@@H]1Cc2c([nH]c3ccccc23)[C@@H](c2c(F)cc(N3CCCC4(CCCN(CC(=O)O)CCC4)C3)cc2F)N1CC(F)F] |
| 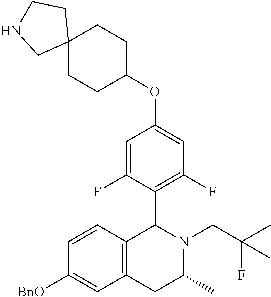 [SMILES: C[C@@H]1Cc2cc(OCc3ccccc3)ccc2C(c2c(F)cc(OC3CCC4(CCNC4)CC3)cc2F)N1CC(C)(C)F] | 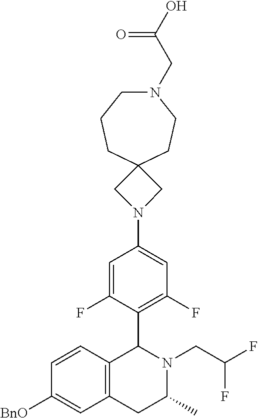 [SMILES: C[C@@H]1Cc2cc(OCc3ccccc3)ccc2C(c2c(F)cc(N3CC4(CCCN(CC(=O)O)CC4)C3)cc2F)N1CC(F)F] | 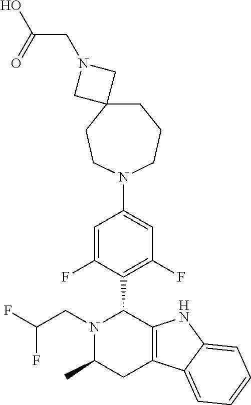 [SMILES: C[C@@H]1Cc2c([nH]c3ccccc23)[C@@H](c2c(F)cc(N3CCCC4(CC3)CN(CC(=O)O)C4)cc2F)N1CC(F)F] |
| 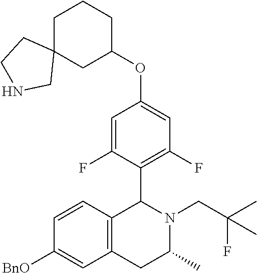 [SMILES: C[C@@H]1Cc2cc(OCc3ccccc3)ccc2C(c2c(F)cc(OC3CCCC4(CCNC4)C3)cc2F)N1CC(C)(C)F] | 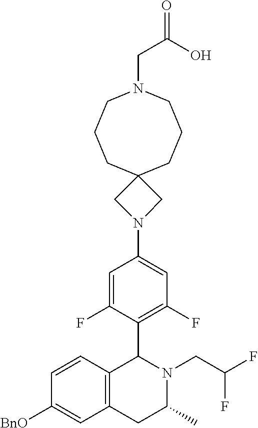 [SMILES: C[C@@H]1Cc2cc(OCc3ccccc3)ccc2C(c2c(F)cc(N3CC4(CCCN(CC(=O)O)CCC4)C3)cc2F)N1CC(F)F] | 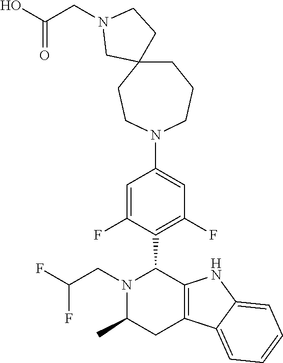 [SMILES: C[C@@H]1Cc2c([nH]c3ccccc23)[C@@H](c2c(F)cc(N3CCCC4(CCN(CC(=O)O)C4)CC3)cc2F)N1CC(F)F] |
| 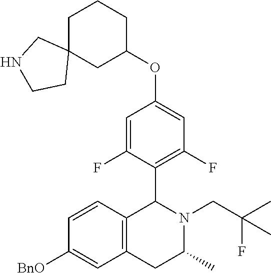 [SMILES: C[C@@H]1Cc2cc(OCc3ccccc3)ccc2C(c2c(F)cc(OC3CCCC4(CCNC4)C3)cc2F)N1CC(C)(C)F] | 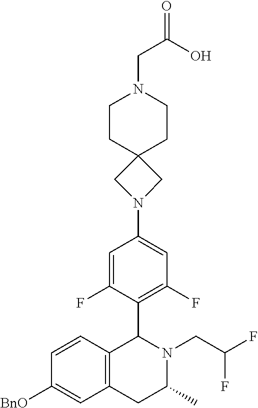 [SMILES: C[C@@H]1Cc2cc(OCc3ccccc3)ccc2C(c2c(F)cc(N3CC4(CCN(CC(=O)O)CC4)C3)cc2F)N1CC(F)F] | 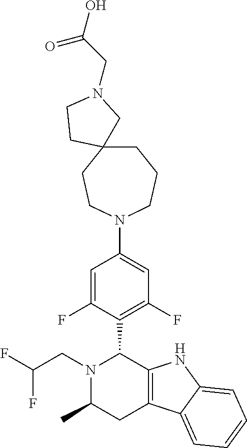 [SMILES: C[C@@H]1Cc2c([nH]c3ccccc23)[C@@H](c2c(F)cc(N3CCCC4(CCN(CC(=O)O)C4)CC3)cc2F)N1CC(F)F] |
| 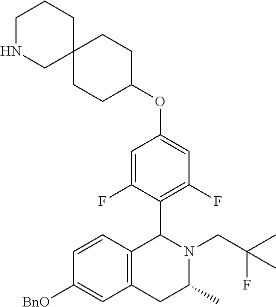 [SMILES: C[C@@H]1Cc2cc(OCc3ccccc3)ccc2C(c2c(F)cc(OC3CCC4(CCCNC4)CC3)cc2F)N1CC(C)(C)F] | 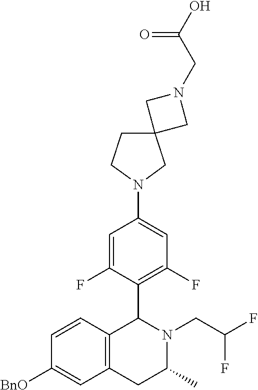 [SMILES: C[C@@H]1Cc2cc(OCc3ccccc3)ccc2C(c2c(F)cc(N3CCC4(CN(CC(=O)O)C4)C3)cc2F)N1CC(F)F] | 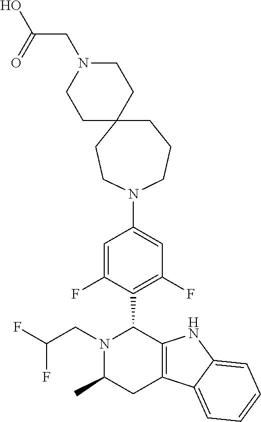 [SMILES: C[C@@H]1Cc2c([nH]c3ccccc23)[C@@H](c2c(F)cc(N3CCCC4(CCN(CC(=O)O)CC4)CC3)cc2F)N1CC(F)F] |
| 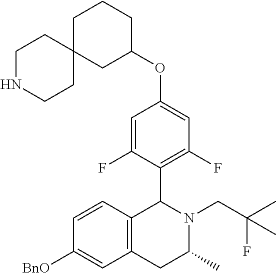 [SMILES: C[C@@H]1Cc2cc(OCc3ccccc3)ccc2C(c2c(F)cc(OC3CCCC4(CCNCC4)C3)cc2F)N1CC(C)(C)F] | 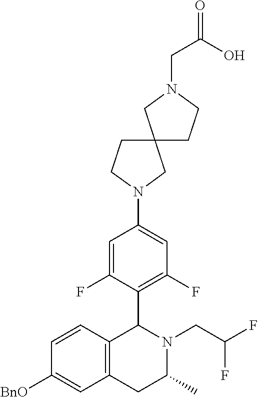 [SMILES: C[C@@H]1Cc2cc(OCc3ccccc3)ccc2C(c2c(F)cc(N3CCC4(CCN(CC(=O)O)C4)C3)cc2F)N1CC(F)F] | 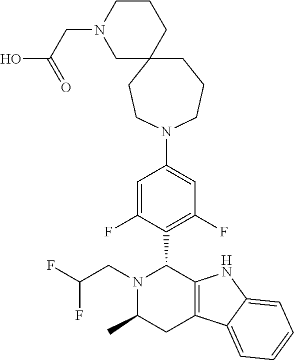 [SMILES: C[C@@H]1Cc2c([nH]c3ccccc23)[C@@H](c2c(F)cc(N3CCCC4(CCCN(CC(=O)O)C4)CC3)cc2F)N1CC(F)F] |
| 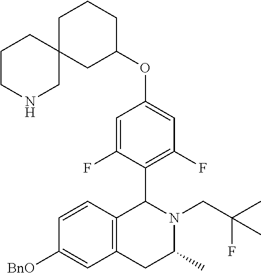 [SMILES: C[C@@H]1Cc2cc(OCc3ccccc3)ccc2C(c2c(F)cc(OC3CCCC4(CCCNC4)C3)cc2F)N1CC(C)(C)F] | 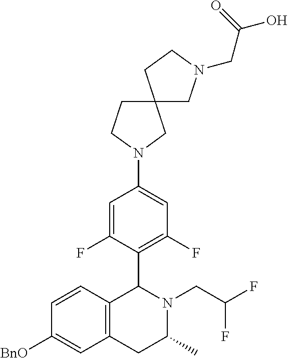 [SMILES: C[C@@H]1Cc2cc(OCc3ccccc3)ccc2C(c2c(F)cc(N3CCC4(CCN(CC(=O)O)C4)C3)cc2F)N1CC(F)F] | 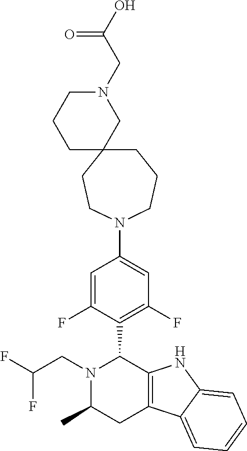 [SMILES: C[C@@H]1Cc2c([nH]c3ccccc23)[C@@H](c2c(F)cc(N3CCCC4(CCCN(CC(=O)O)C4)CC3)cc2F)N1CC(F)F] |
| 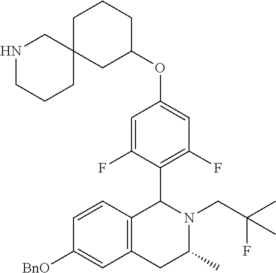 [SMILES: C[C@@H]1Cc2cc(OCc3ccccc3)ccc2C(c2c(F)cc(OC3CCCC4(CCCNC4)C3)cc2F)N1CC(C)(C)F] | 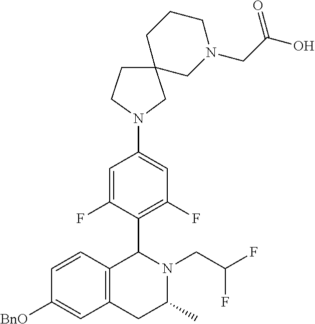 [SMILES: C[C@@H]1Cc2cc(OCc3ccccc3)ccc2C(c2c(F)cc(N3CCC4(CCCN(CC(=O)O)C4)C3)cc2F)N1CC(F)F] | 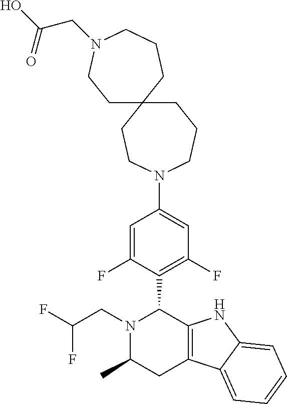 [SMILES: C[C@@H]1Cc2c([nH]c3ccccc23)[C@@H](c2c(F)cc(N3CCCC4(CCCN(CC(=O)O)CC4)CC3)cc2F)N1CC(F)F] |
| 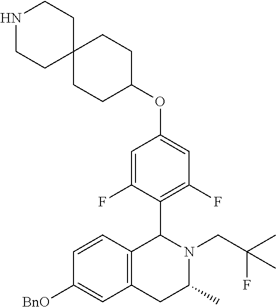 [SMILES: C[C@@H]1Cc2cc(OCc3ccccc3)ccc2C(c2c(F)cc(OC3CCC4(CCNCC4)CC3)cc2F)N1CC(C)(C)F] | 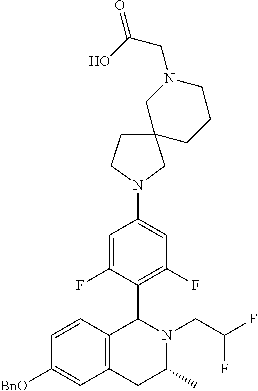 [SMILES: C[C@@H]1Cc2cc(OCc3ccccc3)ccc2C(c2c(F)cc(N3CCC4(CCCN(CC(=O)O)C4)C3)cc2F)N1CC(F)F] | 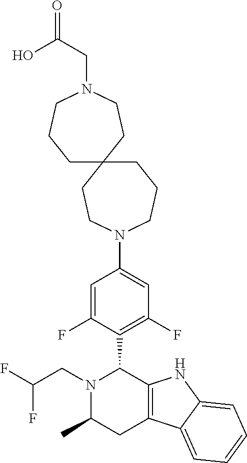 [SMILES: C[C@@H]1Cc2c([nH]c3ccccc23)[C@@H](c2c(F)cc(N3CCCC4(CCCN(CC(=O)O)CC4)CC3)cc2F)N1CC(F)F] |
| 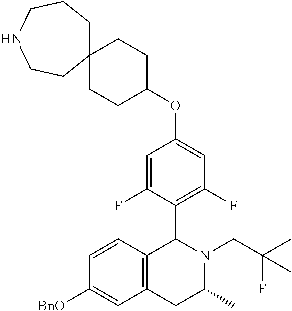 [SMILES: C[C@@H]1Cc2cc(OCc3ccccc3)ccc2C(c2c(F)cc(OC3CCC4(CCCNCC4)CC3)cc2F)N1CC(C)(C)F] | 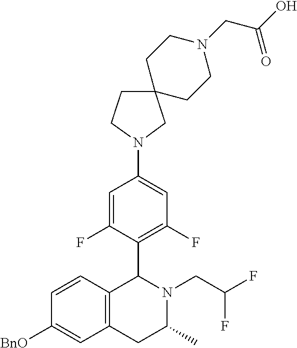 [SMILES: C[C@@H]1Cc2cc(OCc3ccccc3)ccc2C(c2c(F)cc(N3CCC4(CCN(CC(=O)O)CC4)C3)cc2F)N1CC(F)F] | 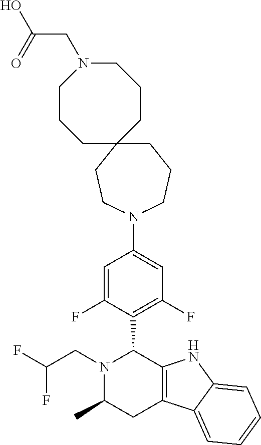 [SMILES: C[C@@H]1Cc2c([nH]c3ccccc23)[C@@H](c2c(F)cc(N3CCCC4(CCCN(CC(=O)O)CCC4)CC3)cc2F)N1CC(F)F] |
| 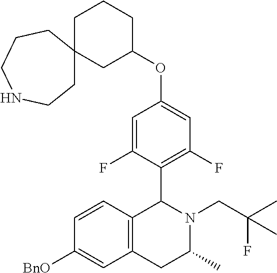 [SMILES: C[C@@H]1Cc2cc(OCc3ccccc3)ccc2C(c2c(F)cc(OC3CCCC4(CCCNCC4)C3)cc2F)N1CC(C)(C)F] | 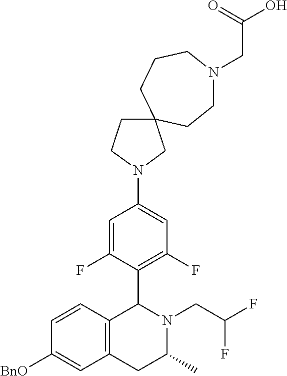 [SMILES: C[C@@H]1Cc2cc(OCc3ccccc3)ccc2C(c2c(F)cc(N3CCC4(CCCN(CC(=O)O)CC4)C3)cc2F)N1CC(F)F] | 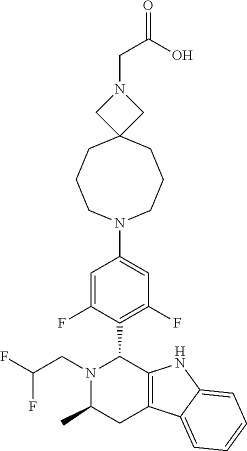 [SMILES: C[C@@H]1Cc2c([nH]c3ccccc23)[C@@H](c2c(F)cc(N3CCCC4(CCC3)CN(CC(=O)O)C4)cc2F)N1CC(F)F] |
| 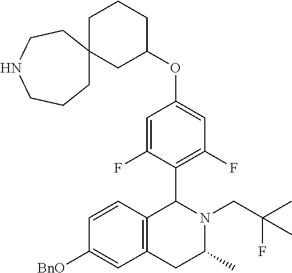 [SMILES: C[C@@H]1Cc2cc(OCc3ccccc3)ccc2C(c2c(F)cc(OC3CCCC4(CCCNCC4)C3)cc2F)N1CC(C)(C)F] | 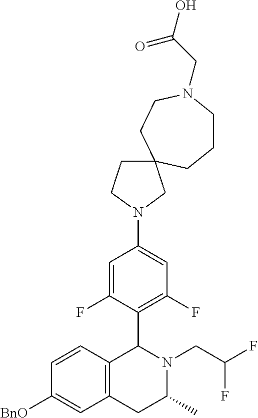 [SMILES: C[C@@H]1Cc2cc(OCc3ccccc3)ccc2C(c2c(F)cc(N3CCC4(CCCN(CC(=O)O)CC4)C3)cc2F)N1CC(F)F] | 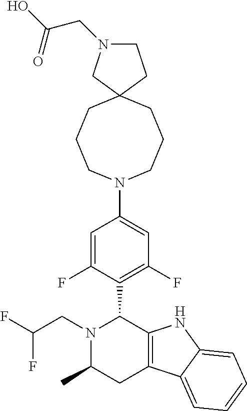 [SMILES: C[C@@H]1Cc2c([nH]c3ccccc23)[C@@H](c2c(F)cc(N3CCCC4(CCC3)CCN(CC(=O)O)C4)cc2F)N1CC(F)F] |
| 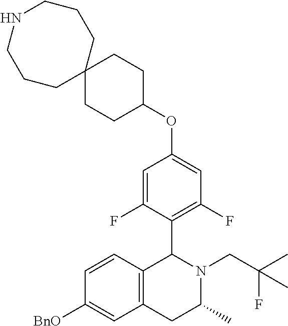 [SMILES: C[C@@H]1Cc2cc(OCc3ccccc3)ccc2C(c2c(F)cc(OC3CCC4(CCCNCCC4)CC3)cc2F)N1CC(C)(C)F] | 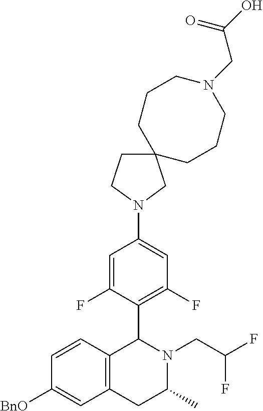 [SMILES: C[C@@H]1Cc2cc(OCc3ccccc3)ccc2C(c2c(F)cc(N3CCC4(CCCN(CC(=O)O)CCC4)C3)cc2F)N1CC(F)F] | 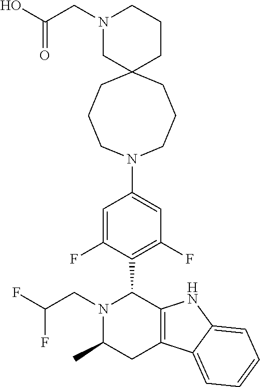 [SMILES: C[C@@H]1Cc2c([nH]c3ccccc23)[C@@H](c2c(F)cc(N3CCCC4(CCCN(CC(=O)O)C4)CCC3)cc2F)N1CC(F)F] |
| 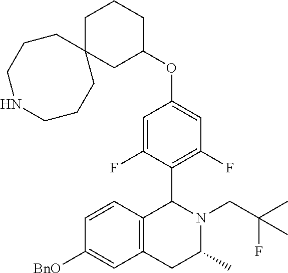 [SMILES: C[C@@H]1Cc2cc(OCc3ccccc3)ccc2C(c2c(F)cc(OC3CCCC4(CCCNCCC4)C3)cc2F)N1CC(C)(C)F] | 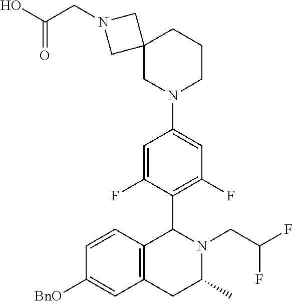 [SMILES: C[C@@H]1Cc2cc(OCc3ccccc3)ccc2C(c2c(F)cc(N3CCCC4(CN(CC(=O)O)C4)C3)cc2F)N1CC(F)F] | 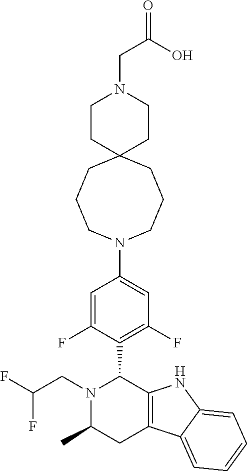 [SMILES: C[C@@H]1Cc2c([nH]c3ccccc23)[C@@H](c2c(F)cc(N3CCCC4(CCC3)CCN(CC(=O)O)CC4)cc2F)N1CC(F)F] |
| 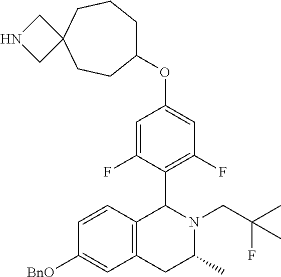 [SMILES: C[C@@H]1Cc2cc(OCc3ccccc3)ccc2C(c2c(F)cc(OC3CCCC4(CC3)CNC4)cc2F)N1CC(C)(C)F] | 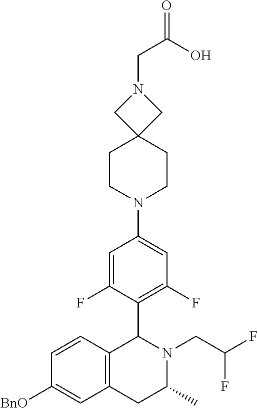 [SMILES: C[C@@H]1Cc2cc(OCc3ccccc3)ccc2C(c2c(F)cc(N3CCC4(CC3)CN(CC(=O)O)C4)cc2F)N1CC(F)F] | 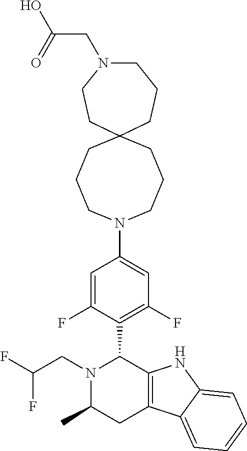 [SMILES: C[C@@H]1Cc2c([nH]c3ccccc23)[C@@H](c2c(F)cc(N3CCCC4(CCCN(CC(=O)O)CC4)CCC3)cc2F)N1CC(F)F] |
| 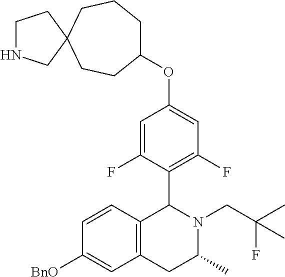 [SMILES: C[C@@H]1Cc2cc(OCc3ccccc3)ccc2C(c2c(F)cc(OC3CCCC4(CCNC4)CC3)cc2F)N1CC(C)(C)F] | 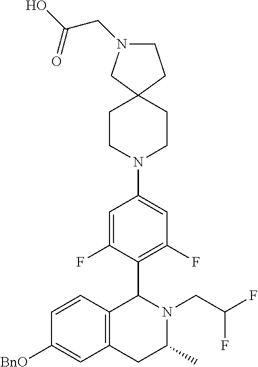 [SMILES: C[C@@H]1Cc2cc(OCc3ccccc3)ccc2C(c2c(F)cc(N3CCC4(CCN(CC(=O)O)C4)CC3)cc2F)N1CC(F)F] | 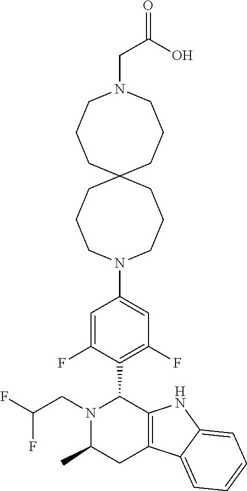 [SMILES: C[C@@H]1Cc2c([nH]c3ccccc23)[C@@H](c2c(F)cc(N3CCCC4(CCCN(CC(=O)O)CCC4)CCC3)cc2F)N1CC(F)F] |
| 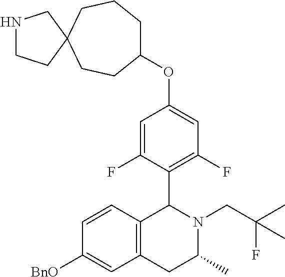 [SMILES: C[C@@H]1Cc2cc(OCc3ccccc3)ccc2C(c2c(F)cc(OC3CCCC4(CCNC4)CC3)cc2F)N1CC(C)(C)F] | 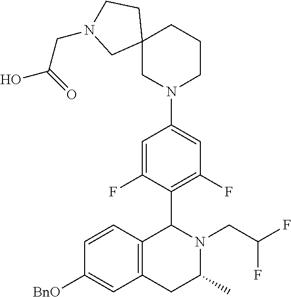 [SMILES: C[C@@H]1Cc2cc(OCc3ccccc3)ccc2C(c2c(F)cc(N3CCCC4(CCN(CC(=O)O)C4)C3)cc2F)N1CC(F)F] | 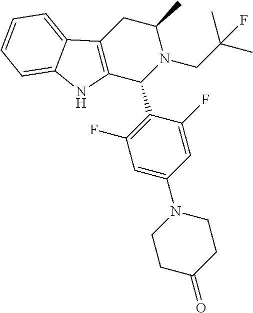 [SMILES: C[C@@H]1Cc2c([nH]c3ccccc23)[C@@H](c2c(F)cc(N3CCC(=O)CC3)cc2F)N1CC(C)(C)F] |
| 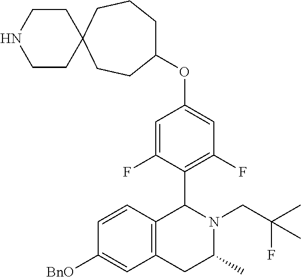 [SMILES: C[C@@H]1Cc2cc(OCc3ccccc3)ccc2C(c2c(F)cc(OC3CCCC4(CCNCC4)CC3)cc2F)N1CC(C)(C)F] | 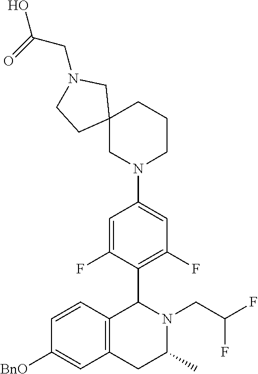 [SMILES: C[C@@H]1Cc2cc(OCc3ccccc3)ccc2C(c2c(F)cc(N3CCCC4(CCN(CC(=O)O)C4)C3)cc2F)N1CC(F)F] | 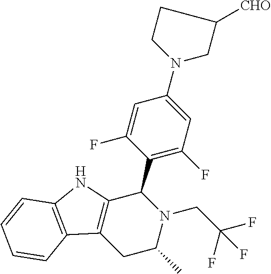 [SMILES: C[C@@H]1Cc2c([nH]c3ccccc23)[C@@H](c2c(F)cc(N3CCC(C=O)C3)cc2F)N1CC(F)(F)F] |
| 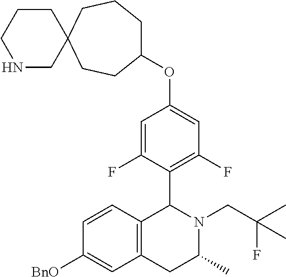 [SMILES: C[C@@H]1Cc2cc(OCc3ccccc3)ccc2C(c2c(F)cc(OC3CCCC4(CCCNC4)CC3)cc2F)N1CC(C)(C)F] | 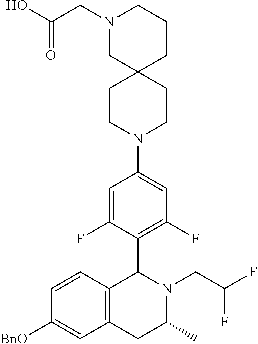 [SMILES: C[C@@H]1Cc2cc(OCc3ccccc3)ccc2C(c2c(F)cc(N3CCC4(CCCN(CC(=O)O)C4)CC3)cc2F)N1CC(F)F] | 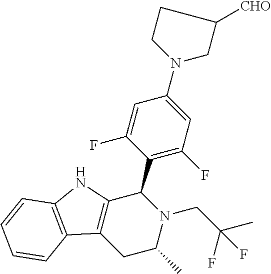 [SMILES: C[C@@H]1Cc2c([nH]c3ccccc23)[C@@H](c2c(F)cc(N3CCC(C=O)C3)cc2F)N1CC(C)(F)F] |
| 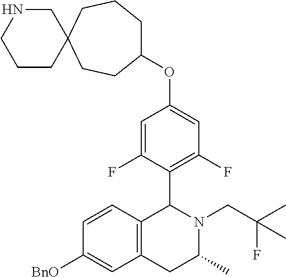 [SMILES: C[C@@H]1Cc2cc(OCc3ccccc3)ccc2C(c2c(F)cc(OC3CCCC4(CCCNC4)CC3)cc2F)N1CC(C)(C)F] | 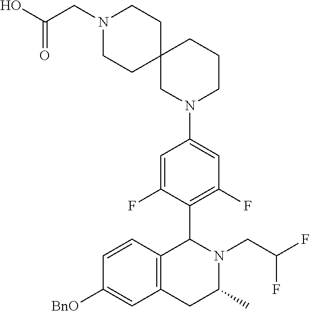 [SMILES: C[C@@H]1Cc2cc(OCc3ccccc3)ccc2C(c2c(F)cc(N3CCCC4(CCN(CC(=O)O)CC4)C3)cc2F)N1CC(F)F] | 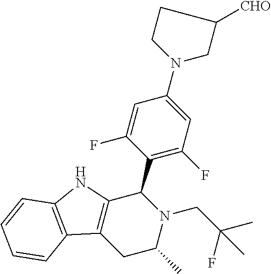 [SMILES: C[C@@H]1Cc2c([nH]c3ccccc23)[C@@H](c2c(F)cc(N3CCC(C=O)C3)cc2F)N1CC(C)(C)F] |
| 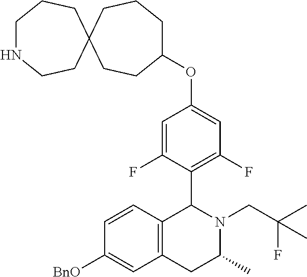 [SMILES: C[C@@H]1Cc2cc(OCc3ccccc3)ccc2C(c2c(F)cc(OC3CCCC4(CCCNCC4)CC3)cc2F)N1CC(C)(C)F] | 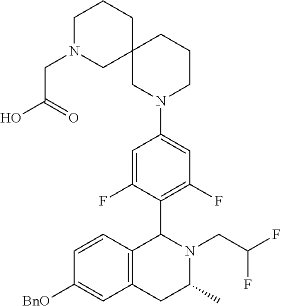 [SMILES: C[C@@H]1Cc2cc(OCc3ccccc3)ccc2C(c2c(F)cc(N3CCCC4(CCCN(CC(=O)O)C4)C3)cc2F)N1CC(F)F] | 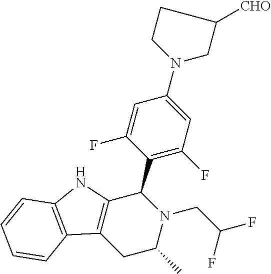 [SMILES: C[C@@H]1Cc2c([nH]c3ccccc23)[C@@H](c2c(F)cc(N3CCC(C=O)C3)cc2F)N1CC(F)F] |
| 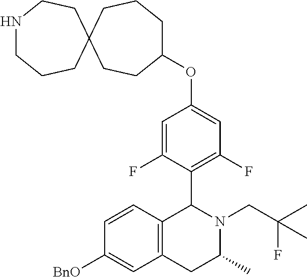 [SMILES: C[C@@H]1Cc2cc(OCc3ccccc3)ccc2C(c2c(F)cc(OC3CCCC4(CCCNCC4)CC3)cc2F)N1CC(C)(C)F] | 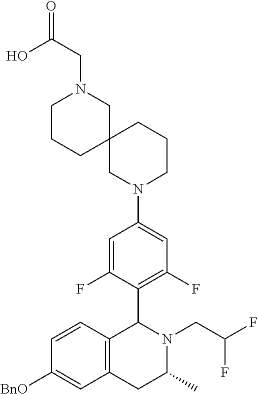 [SMILES: C[C@@H]1Cc2cc(OCc3ccccc3)ccc2C(c2c(F)cc(N3CCCC4(CCCN(CC(=O)O)C4)C3)cc2F)N1CC(F)F] | 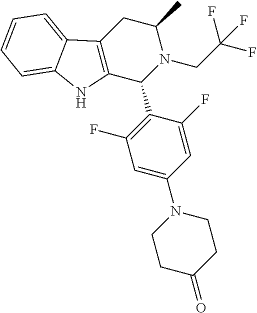 [SMILES: C[C@@H]1Cc2c([nH]c3ccccc23)[C@@H](c2c(F)cc(N3CCC(=O)CC3)cc2F)N1CC(F)(F)F] |
| 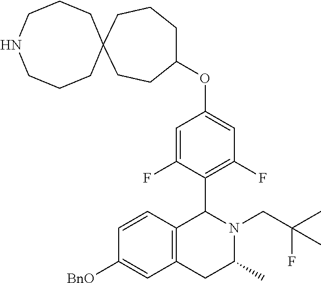 [SMILES: C[C@@H]1Cc2cc(OCc3ccccc3)ccc2C(c2c(F)cc(OC3CCCC4(CCCNCCC4)CC3)cc2F)N1CC(C)(C)F] | 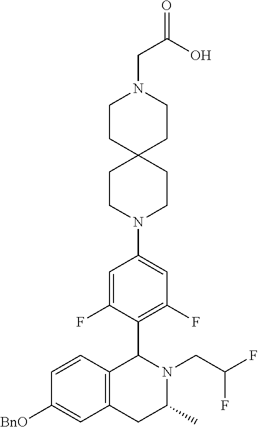 [SMILES: C[C@@H]1Cc2cc(OCc3ccccc3)ccc2C(c2c(F)cc(N3CCC4(CCN(CC(=O)O)CC4)CC3)cc2F)N1CC(F)F] | 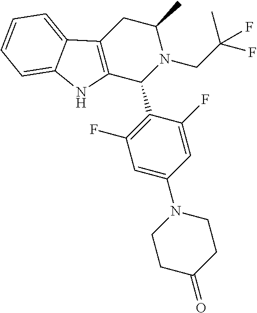 [SMILES: C[C@@H]1Cc2c([nH]c3ccccc23)[C@@H](c2c(F)cc(N3CCC(=O)CC3)cc2F)N1CC(C)(F)F] |
| 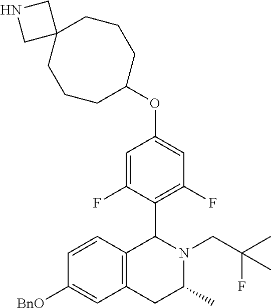 [SMILES: C[C@@H]1Cc2cc(OCc3ccccc3)ccc2C(c2c(F)cc(OC3CCCC4(CCC3)CNC4)cc2F)N1CC(C)(C)F] | 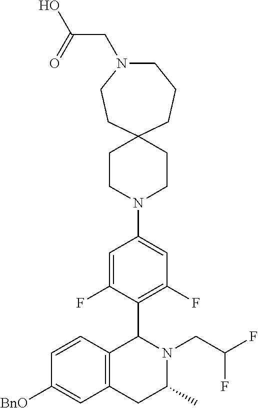 [SMILES: C[C@@H]1Cc2cc(OCc3ccccc3)ccc2C(c2c(F)cc(N3CCC4(CCCN(CC(=O)O)CC4)CC3)cc2F)N1CC(F)F] | 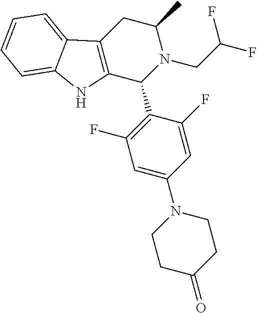 [SMILES: C[C@@H]1Cc2c([nH]c3ccccc23)[C@@H](c2c(F)cc(N3CCC(=O)CC3)cc2F)N1CC(F)F] |
| 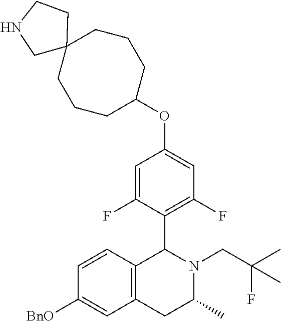 [SMILES: C[C@@H]1Cc2cc(OCc3ccccc3)ccc2C(c2c(F)cc(OC3CCCC4(CCC3)CCNC4)cc2F)N1CC(C)(C)F] | 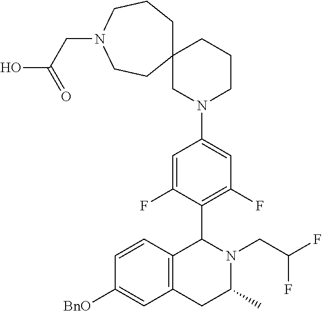 [SMILES: C[C@@H]1Cc2cc(OCc3ccccc3)ccc2C(c2c(F)cc(N3CCCC4(CCCN(CC(=O)O)CC4)C3)cc2F)N1CC(F)F] | 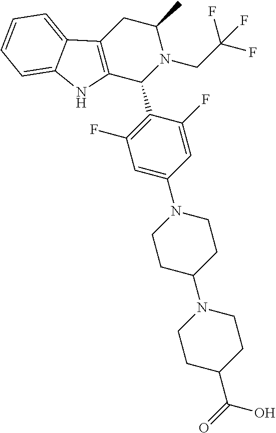 [SMILES: C[C@@H]1Cc2c([nH]c3ccccc23)[C@@H](c2c(F)cc(N3CCC(N4CCC(C(=O)O)CC4)CC3)cc2F)N1CC(F)(F)F] |
| 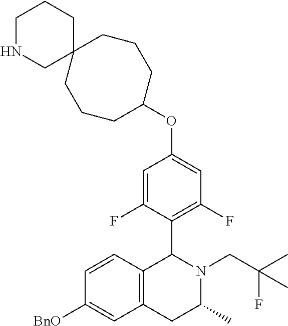 [SMILES: C[C@@H]1Cc2cc(OCc3ccccc3)ccc2C(c2c(F)cc(OC3CCCC4(CCCNC4)CCC3)cc2F)N1CC(C)(C)F] | 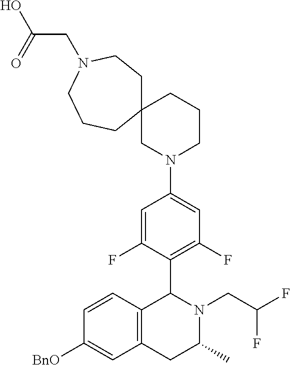 [SMILES: C[C@@H]1Cc2cc(OCc3ccccc3)ccc2C(c2c(F)cc(N3CCCC4(CCCN(CC(=O)O)CC4)C3)cc2F)N1CC(F)F] | 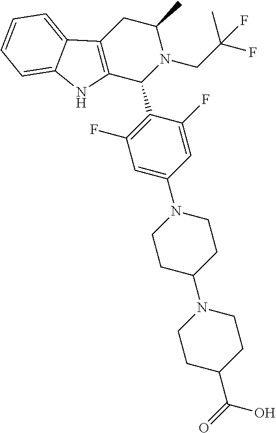 [SMILES: C[C@@H]1Cc2c([nH]c3ccccc23)[C@@H](c2c(F)cc(N3CCC(N4CCC(C(=O)O)CC4)CC3)cc2F)N1CC(C)(F)F] |
| 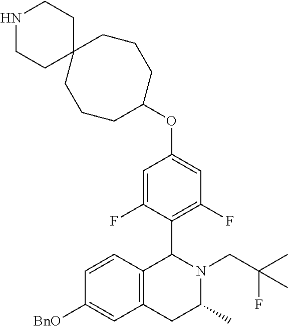 [SMILES: C[C@@H]1Cc2cc(OCc3ccccc3)ccc2C(c2c(F)cc(OC3CCCC4(CCC3)CCNCC4)cc2F)N1CC(C)(C)F] | 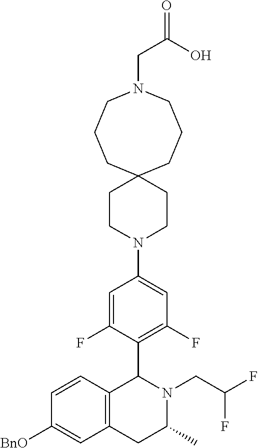 [SMILES: C[C@@H]1Cc2cc(OCc3ccccc3)ccc2C(c2c(F)cc(N3CCC4(CCCN(CC(=O)O)CCC4)CC3)cc2F)N1CC(F)F] | 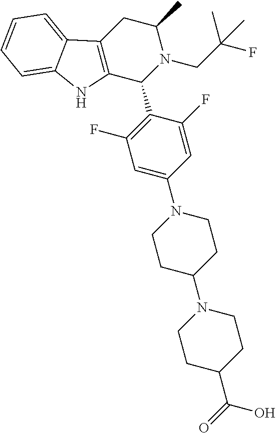 [SMILES: C[C@@H]1Cc2c([nH]c3ccccc23)[C@@H](c2c(F)cc(N3CCC(N4CCC(C(=O)O)CC4)CC3)cc2F)N1CC(C)(C)F] |
| 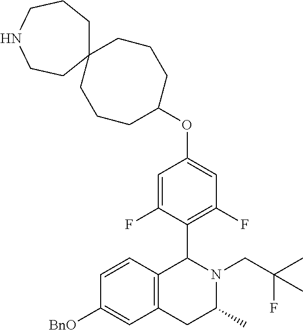 [SMILES: C[C@@H]1Cc2cc(OCc3ccccc3)ccc2C(c2c(F)cc(OC3CCCC4(CCCNCC4)CCC3)cc2F)N1CC(C)(C)F] | 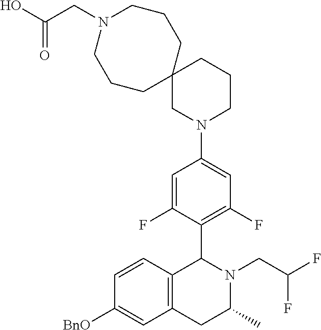 [SMILES: C[C@@H]1Cc2cc(OCc3ccccc3)ccc2C(c2c(F)cc(N3CCCC4(CCCN(CC(=O)O)CCC4)C3)cc2F)N1CC(F)F] | 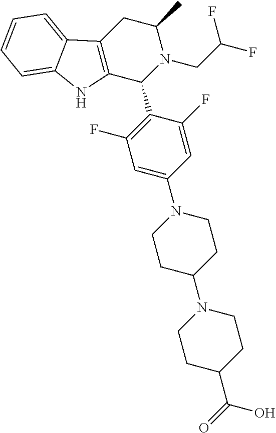 [SMILES: C[C@@H]1Cc2c([nH]c3ccccc23)[C@@H](c2c(F)cc(N3CCC(N4CCC(C(=O)O)CC4)CC3)cc2F)N1CC(F)F] |
| 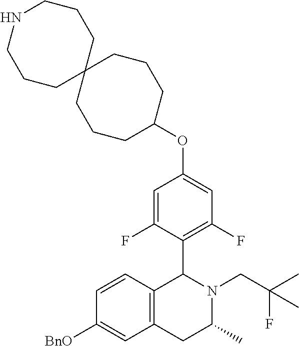 [SMILES: C[C@@H]1Cc2cc(OCc3ccccc3)ccc2C(c2c(F)cc(OC3CCCC4(CCCNCCC4)CCC3)cc2F)N1CC(C)(C)F] | 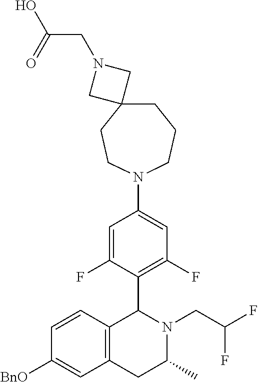 [SMILES: C[C@@H]1Cc2cc(OCc3ccccc3)ccc2C(c2c(F)cc(N3CCCC4(CC3)CN(CC(=O)O)C4)cc2F)N1CC(F)F] | 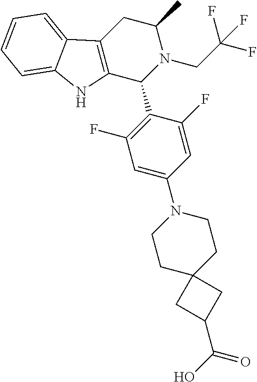 [SMILES: C[C@@H]1Cc2c([nH]c3ccccc23)[C@@H](c2c(F)cc(N3CCC4(CC3)CC(C(=O)O)C4)cc2F)N1CC(F)(F)F] |
| 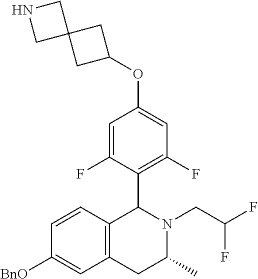 [SMILES: C[C@@H]1Cc2cc(OCc3ccccc3)ccc2C(c2c(F)cc(OC3CC4(CNC4)C3)cc2F)N1CC(F)F] | 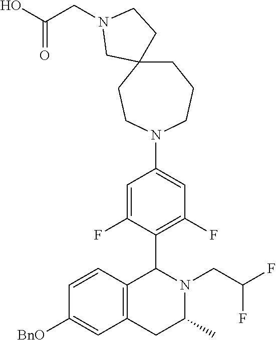 [SMILES: C[C@@H]1Cc2cc(OCc3ccccc3)ccc2C(c2c(F)cc(N3CCCC4(CCN(CC(=O)O)C4)CC3)cc2F)N1CC(F)F] | 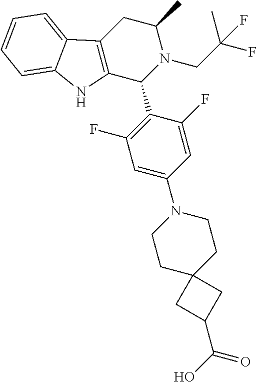 [SMILES: C[C@@H]1Cc2c([nH]c3ccccc23)[C@@H](c2c(F)cc(N3CCC4(CC3)CC(C(=O)O)C4)cc2F)N1CC(C)(F)F] |
| 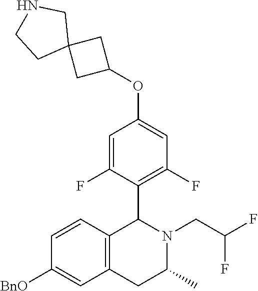 [SMILES: C[C@@H]1Cc2cc(OCc3ccccc3)ccc2C(c2c(F)cc(OC3CC4(CCNC4)C3)cc2F)N1CC(F)F] | 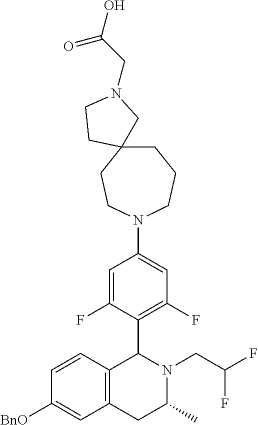 [SMILES: C[C@@H]1Cc2cc(OCc3ccccc3)ccc2C(c2c(F)cc(N3CCCC4(CCN(CC(=O)O)C4)CC3)cc2F)N1CC(F)F] | 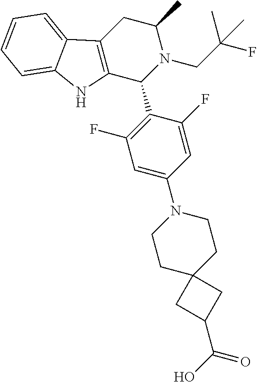 [SMILES: C[C@@H]1Cc2c([nH]c3ccccc23)[C@@H](c2c(F)cc(N3CCC4(CC3)CC(C(=O)O)C4)cc2F)N1CC(C)(C)F] |
| 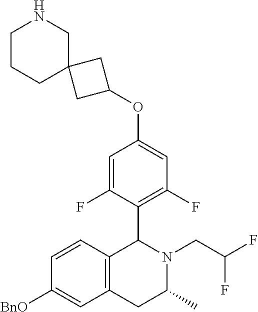 [SMILES: C[C@@H]1Cc2cc(OCc3ccccc3)ccc2C(c2c(F)cc(OC3CC4(CCCNC4)C3)cc2F)N1CC(F)F] | 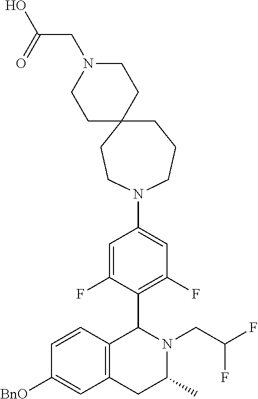 [SMILES: C[C@@H]1Cc2cc(OCc3ccccc3)ccc2C(c2c(F)cc(N3CCCC4(CCN(CC(=O)O)CC4)CC3)cc2F)N1CC(F)F] | 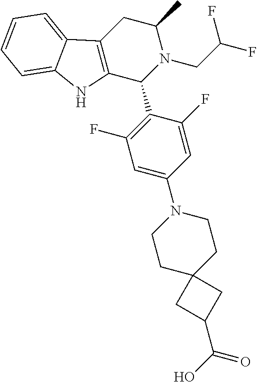 [SMILES: C[C@@H]1Cc2c([nH]c3ccccc23)[C@@H](c2c(F)cc(N3CCC4(CC3)CC(C(=O)O)C4)cc2F)N1CC(F)F] |
| 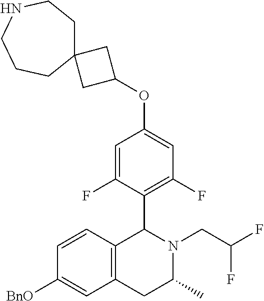 [SMILES: C[C@@H]1Cc2cc(OCc3ccccc3)ccc2C(c2c(F)cc(OC3CC4(CCCNCC4)C3)cc2F)N1CC(F)F] | 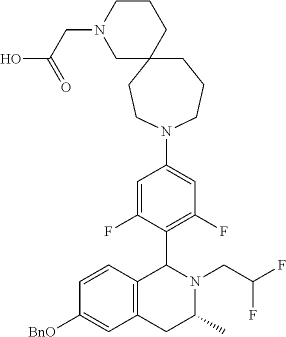 [SMILES: C[C@@H]1Cc2cc(OCc3ccccc3)ccc2C(c2c(F)cc(N3CCCC4(CCCN(CC(=O)O)C4)CC3)cc2F)N1CC(F)F] | 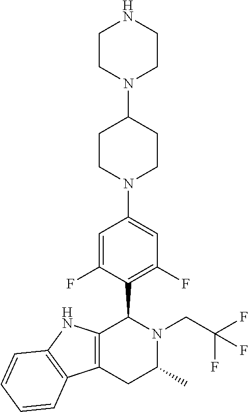 [SMILES: C[C@@H]1Cc2c([nH]c3ccccc23)[C@@H](c2c(F)cc(N3CCC(N4CCNCC4)CC3)cc2F)N1CC(F)(F)F] |
| 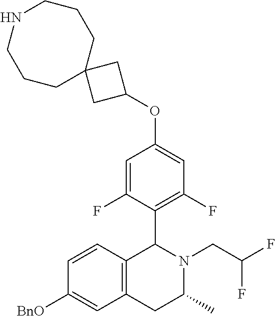 [SMILES: C[C@@H]1Cc2cc(OCc3ccccc3)ccc2C(c2c(F)cc(OC3CC4(CCCNCCC4)C3)cc2F)N1CC(F)F] | 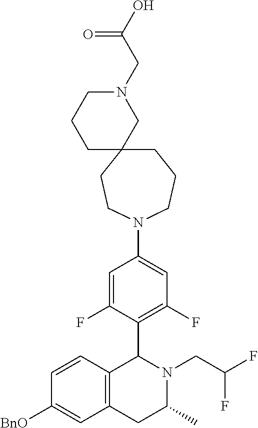 [SMILES: C[C@@H]1Cc2cc(OCc3ccccc3)ccc2C(c2c(F)cc(N3CCCC4(CCCN(CC(=O)O)C4)CC3)cc2F)N1CC(F)F] | 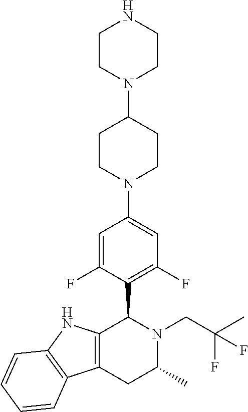 [SMILES: C[C@@H]1Cc2c([nH]c3ccccc23)[C@@H](c2c(F)cc(N3CCC(N4CCNCC4)CC3)cc2F)N1CC(C)(F)F] |
| 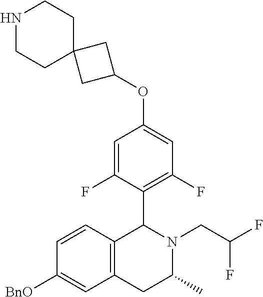 [SMILES: C[C@@H]1Cc2cc(OCc3ccccc3)ccc2C(c2c(F)cc(OC3CC4(CCNCC4)C3)cc2F)N1CC(F)F] | 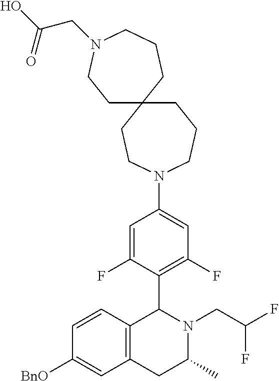 [SMILES: C[C@@H]1Cc2cc(OCc3ccccc3)ccc2C(c2c(F)cc(N3CCCC4(CCCN(CC(=O)O)CC4)CC3)cc2F)N1CC(F)F] | 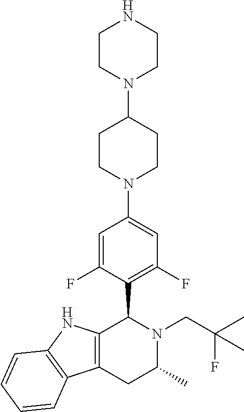 [SMILES: C[C@@H]1Cc2c([nH]c3ccccc23)[C@@H](c2c(F)cc(N3CCC(N4CCNCC4)CC3)cc2F)N1CC(C)(C)F] |
| 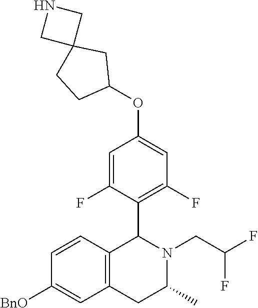 [SMILES: C[C@@H]1Cc2cc(OCc3ccccc3)ccc2C(c2c(F)cc(OC3CCC4(CNC4)C3)cc2F)N1CC(F)F] | 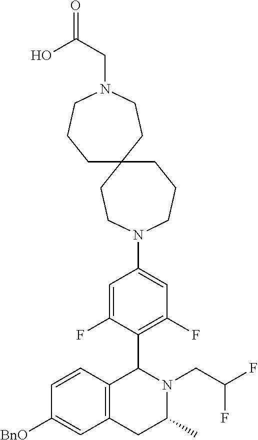 [SMILES: C[C@@H]1Cc2cc(OCc3ccccc3)ccc2C(c2c(F)cc(N3CCCC4(CCCN(CC(=O)O)CC4)CC3)cc2F)N1CC(F)F] | 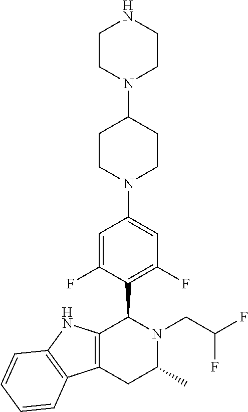 [SMILES: C[C@@H]1Cc2c([nH]c3ccccc23)[C@@H](c2c(F)cc(N3CCC(N4CCNCC4)CC3)cc2F)N1CC(F)F] |
| 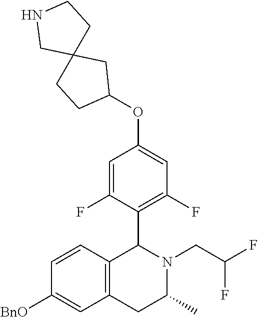 [SMILES: C[C@@H]1Cc2cc(OCc3ccccc3)ccc2C(c2c(F)cc(OC3CCC4(CCNC4)C3)cc2F)N1CC(F)F] | 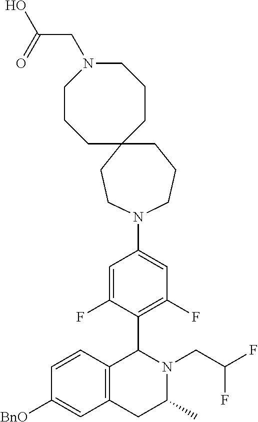 [SMILES: C[C@@H]1Cc2cc(OCc3ccccc3)ccc2C(c2c(F)cc(N3CCCC4(CCCN(CC(=O)O)CCC4)CC3)cc2F)N1CC(F)F] | 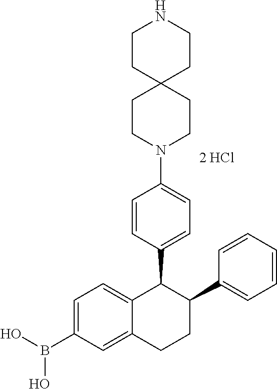 [SMILES: OB(O)c1ccc2c(c1)CC[C@H](c1ccccc1)[C@@H]2c1ccc(N2CCC3(CCNCC3)CC2)cc1] |
| 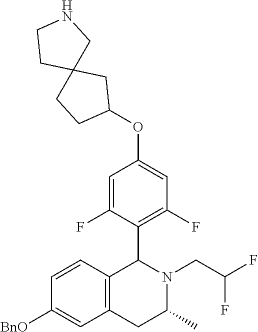 [SMILES: C[C@@H]1Cc2cc(OCc3ccccc3)ccc2C(c2c(F)cc(OC3CCC4(CCNC4)C3)cc2F)N1CC(F)F] | 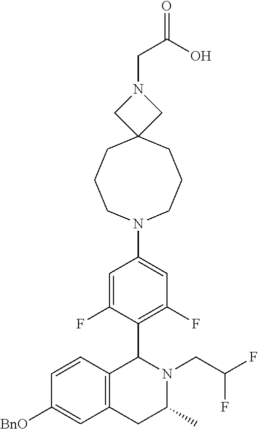 [SMILES: C[C@@H]1Cc2cc(OCc3ccccc3)ccc2C(c2c(F)cc(N3CCCC4(CCC3)CN(CC(=O)O)C4)cc2F)N1CC(F)F] | 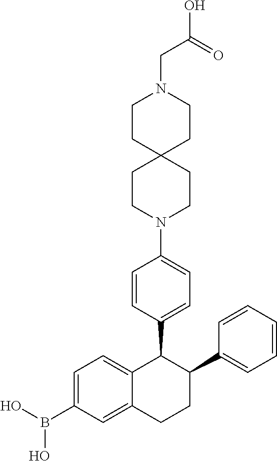 [SMILES: O=C(O)CN1CCC2(CC1)CCN(c1ccc([C@@H]3c4ccc(B(O)O)cc4CC[C@@H]3c3ccccc3)cc1)CC2] |
| 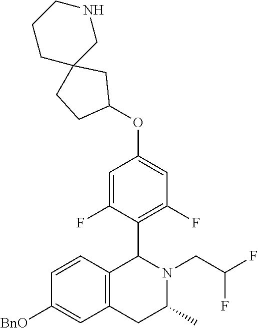 [SMILES: C[C@@H]1Cc2cc(OCc3ccccc3)ccc2C(c2c(F)cc(OC3CCC4(CCCNC4)C3)cc2F)N1CC(F)F] | 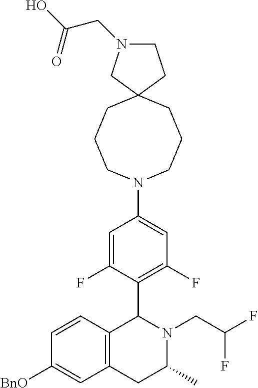 [SMILES: C[C@@H]1Cc2cc(OCc3ccccc3)ccc2C(c2c(F)cc(N3CCCC4(CCC3)CCN(CC(=O)O)C4)cc2F)N1CC(F)F] | |
| 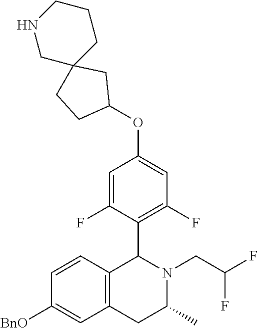 [SMILES: C[C@@H]1Cc2cc(OCc3ccccc3)ccc2C(c2c(F)cc(OC3CCC4(CCCNC4)C3)cc2F)N1CC(F)F] | 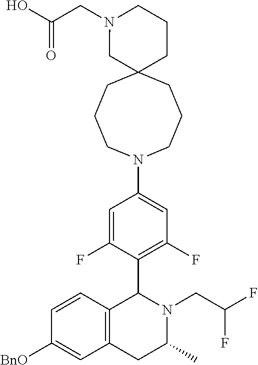 [SMILES: C[C@@H]1Cc2cc(OCc3ccccc3)ccc2C(c2c(F)cc(N3CCCC4(CCCN(CC(=O)O)C4)CCC3)cc2F)N1CC(F)F] | |
| 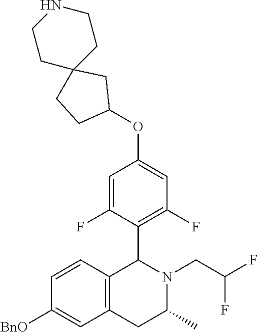 [SMILES: C[C@@H]1Cc2cc(OCc3ccccc3)ccc2C(c2c(F)cc(OC3CCC4(CCNCC4)C3)cc2F)N1CC(F)F] | 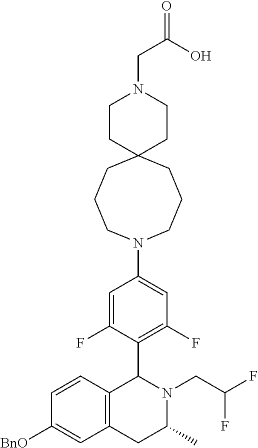 [SMILES: C[C@@H]1Cc2cc(OCc3ccccc3)ccc2C(c2c(F)cc(N3CCCC4(CCC3)CCN(CC(=O)O)CC4)cc2F)N1CC(F)F] | |
| 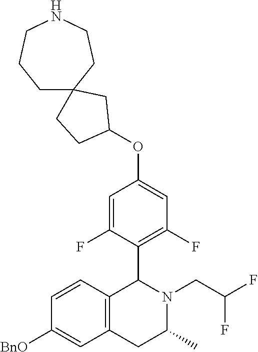 [SMILES: C[C@@H]1Cc2cc(OCc3ccccc3)ccc2C(c2c(F)cc(OC3CCC4(CCCNCC4)C3)cc2F)N1CC(F)F] | 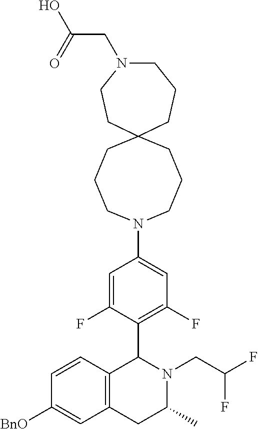 [SMILES: C[C@@H]1Cc2cc(OCc3ccccc3)ccc2C(c2c(F)cc(N3CCCC4(CCCN(CC(=O)O)CC4)CCC3)cc2F)N1CC(F)F] | 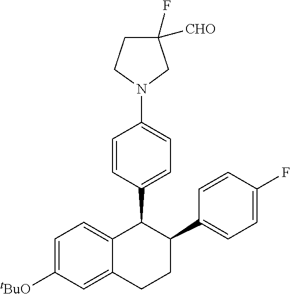 [SMILES: CC(C)(C)Oc1ccc2c(c1)CC[C@H](c1ccc(F)cc1)[C@@H]2c1ccc(N2CCC(F)(C=O)C2)cc1] |
| 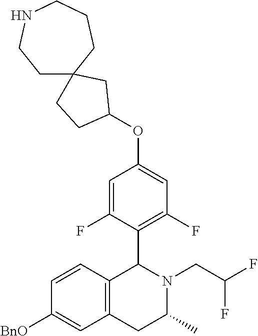 [SMILES: C[C@@H]1Cc2cc(OCc3ccccc3)ccc2C(c2c(F)cc(OC3CCC4(CCCNCC4)C3)cc2F)N1CC(F)F] | 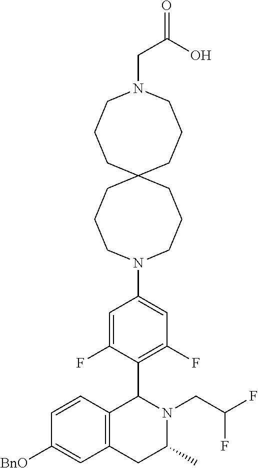 [SMILES: C[C@@H]1Cc2cc(OCc3ccccc3)ccc2C(c2c(F)cc(N3CCCC4(CCCN(CC(=O)O)CCC4)CCC3)cc2F)N1CC(F)F] | 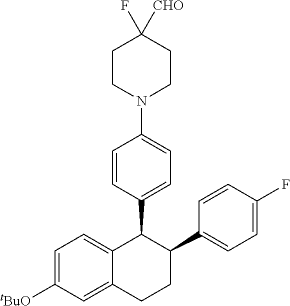 [SMILES: CC(C)(C)Oc1ccc2c(c1)CC[C@H](c1ccc(F)cc1)[C@@H]2c1ccc(N2CCC(F)(C=O)CC2)cc1] |
| 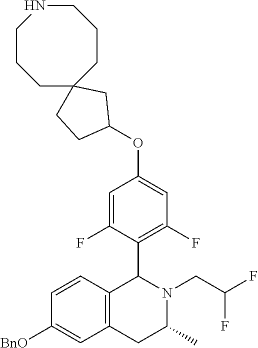 [SMILES: C[C@@H]1Cc2cc(OCc3ccccc3)ccc2C(c2c(F)cc(OC3CCC4(CCCNCCC4)C3)cc2F)N1CC(F)F] | 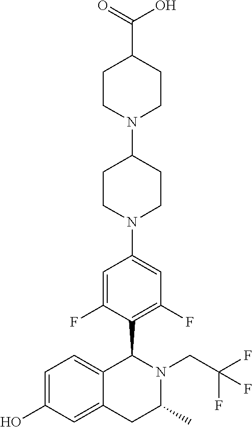 [SMILES: C[C@@H]1Cc2cc(O)ccc2[C@@H](c2c(F)cc(N3CCC(N4CCC(C(=O)O)CC4)CC3)cc2F)N1CC(F)(F)F] | 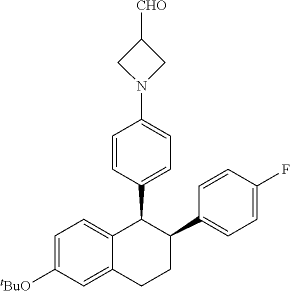 [SMILES: CC(C)(C)Oc1ccc2c(c1)CC[C@H](c1ccc(F)cc1)[C@@H]2c1ccc(N2CC(C=O)C2)cc1] |
| 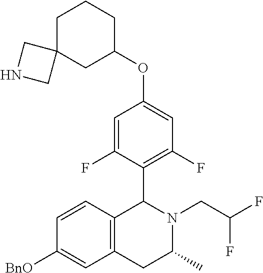 [SMILES: C[C@@H]1Cc2cc(OCc3ccccc3)ccc2C(c2c(F)cc(OC3CCCC4(CNC4)C3)cc2F)N1CC(F)F] | 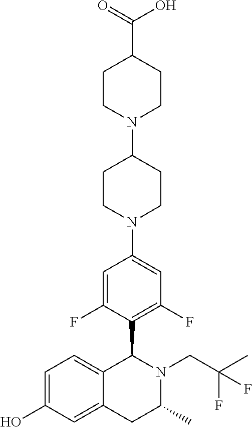 [SMILES: C[C@@H]1Cc2cc(O)ccc2[C@@H](c2c(F)cc(N3CCC(N4CCC(C(=O)O)CC4)CC3)cc2F)N1CC(C)(F)F] | 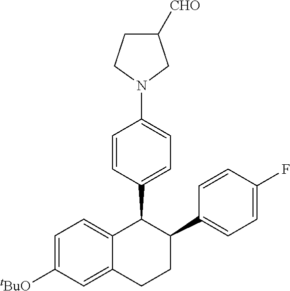 [SMILES: CC(C)(C)Oc1ccc2c(c1)CC[C@H](c1ccc(F)cc1)[C@@H]2c1ccc(N2CCC(C=O)C2)cc1] |
| 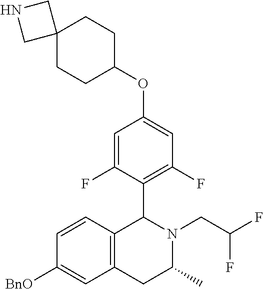 [SMILES: C[C@@H]1Cc2cc(OCc3ccccc3)ccc2C(c2c(F)cc(OC3CCC4(CC3)CNC4)cc2F)N1CC(F)F] | 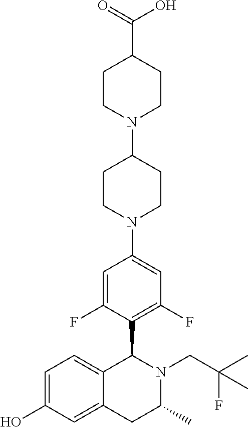 [SMILES: C[C@@H]1Cc2cc(O)ccc2[C@@H](c2c(F)cc(N3CCC(N4CCC(C(=O)O)CC4)CC3)cc2F)N1CC(C)(C)F] | 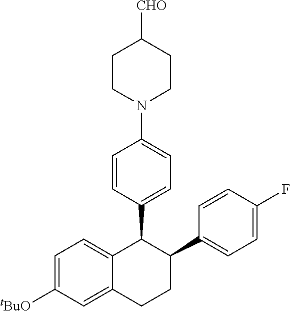 [SMILES: CC(C)(C)Oc1ccc2c(c1)CC[C@H](c1ccc(F)cc1)[C@@H]2c1ccc(N2CCC(C=O)CC2)cc1] |
| 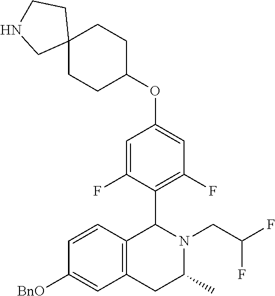 [SMILES: C[C@@H]1Cc2cc(OCc3ccccc3)ccc2C(c2c(F)cc(OC3CCC4(CCNC4)CC3)cc2F)N1CC(F)F] | 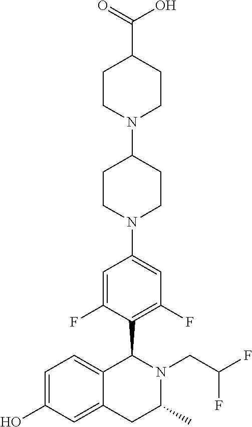 [SMILES: C[C@@H]1Cc2cc(O)ccc2[C@@H](c2c(F)cc(N3CCC(N4CCC(C(=O)O)CC4)CC3)cc2F)N1CC(F)F] | 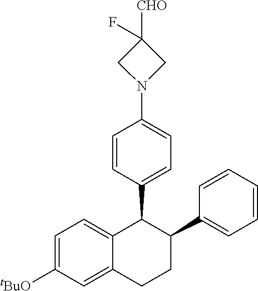 [SMILES: CC(C)(C)Oc1ccc2c(c1)CC[C@H](c1ccccc1)[C@@H]2c1ccc(N2CC(F)(C=O)C2)cc1] |
| 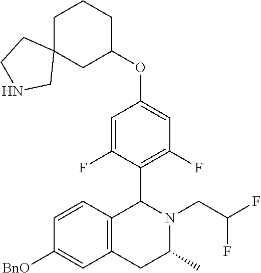 [SMILES: C[C@@H]1Cc2cc(OCc3ccccc3)ccc2C(c2c(F)cc(OC3CCCC4(CCNC4)C3)cc2F)N1CC(F)F] | 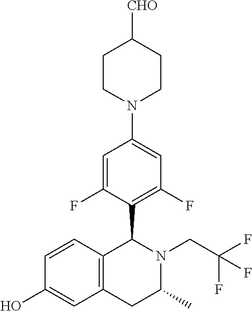 [SMILES: C[C@@H]1Cc2cc(O)ccc2[C@@H](c2c(F)cc(N3CCC(C=O)CC3)cc2F)N1CC(F)(F)F] | 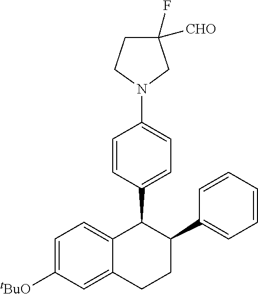 [SMILES: CC(C)(C)Oc1ccc2c(c1)CC[C@H](c1ccccc1)[C@@H]2c1ccc(N2CCC(F)(C=O)C2)cc1] |
| 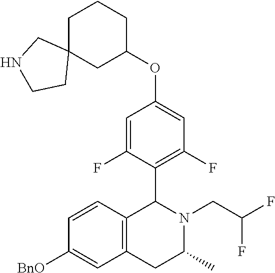 [SMILES: C[C@@H]1Cc2cc(OCc3ccccc3)ccc2C(c2c(F)cc(OC3CCCC4(CCNC4)C3)cc2F)N1CC(F)F] | 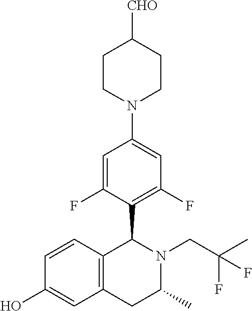 [SMILES: C[C@@H]1Cc2cc(O)ccc2[C@@H](c2c(F)cc(N3CCC(C=O)CC3)cc2F)N1CC(C)(F)F] | 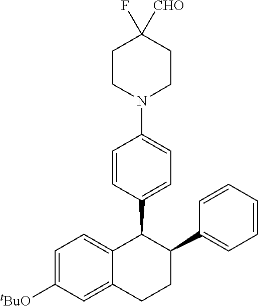 [SMILES: CC(C)(C)Oc1ccc2c(c1)CC[C@H](c1ccccc1)[C@@H]2c1ccc(N2CCC(F)(C=O)CC2)cc1] |
| 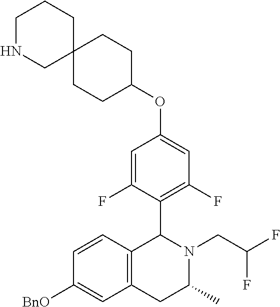 [SMILES: C[C@@H]1Cc2cc(OCc3ccccc3)ccc2C(c2c(F)cc(OC3CCC4(CCCNC4)CC3)cc2F)N1CC(F)F] | 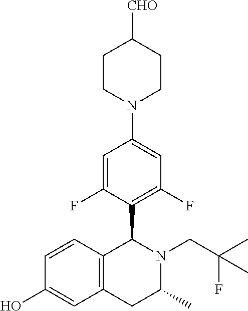 [SMILES: C[C@@H]1Cc2cc(O)ccc2[C@@H](c2c(F)cc(N3CCC(C=O)CC3)cc2F)N1CC(C)(C)F] | 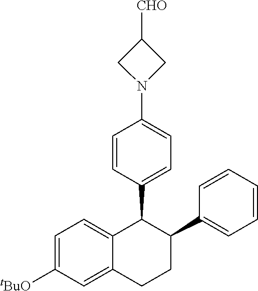 [SMILES: CC(C)(C)Oc1ccc2c(c1)CC[C@H](c1ccccc1)[C@@H]2c1ccc(N2CC(C=O)C2)cc1] |
| 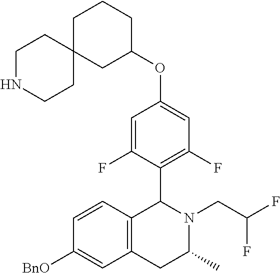 [SMILES: C[C@@H]1Cc2cc(OCc3ccccc3)ccc2C(c2c(F)cc(OC3CCCC4(CCNCC4)C3)cc2F)N1CC(F)F] | 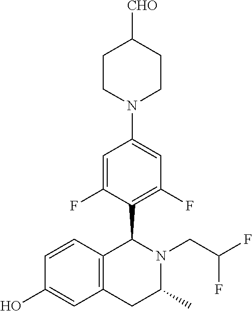 [SMILES: C[C@@H]1Cc2cc(O)ccc2[C@@H](c2c(F)cc(N3CCC(C=O)CC3)cc2F)N1CC(F)F] | 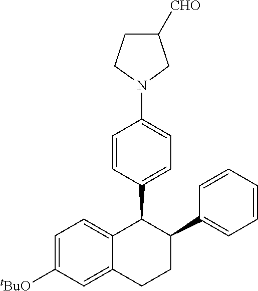 [SMILES: CC(C)(C)Oc1ccc2c(c1)CC[C@H](c1ccccc1)[C@@H]2c1ccc(N2CCC(C=O)C2)cc1] |
| 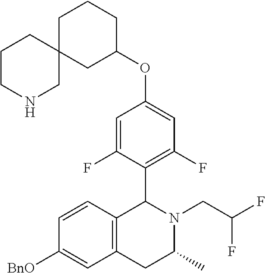 [SMILES: C[C@@H]1Cc2cc(OCc3ccccc3)ccc2C(c2c(F)cc(OC3CCCC4(CCCNC4)C3)cc2F)N1CC(F)F] | 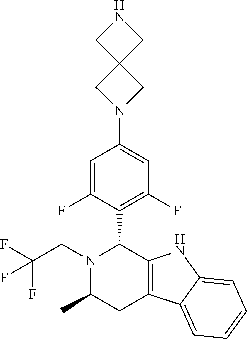 [SMILES: C[C@@H]1Cc2c([nH]c3ccccc23)[C@@H](c2c(F)cc(N3CC4(CNC4)C3)cc2F)N1CC(F)(F)F] | 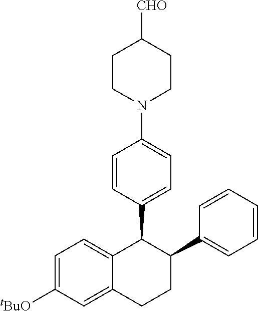 [SMILES: CC(C)(C)Oc1ccc2c(c1)CC[C@H](c1ccccc1)[C@@H]2c1ccc(N2CCC(C=O)CC2)cc1] |
| 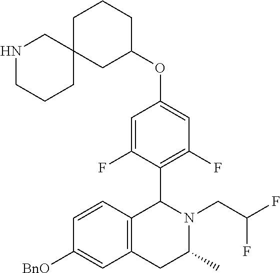 [SMILES: C[C@@H]1Cc2cc(OCc3ccccc3)ccc2C(c2c(F)cc(OC3CCCC4(CCCNC4)C3)cc2F)N1CC(F)F] | 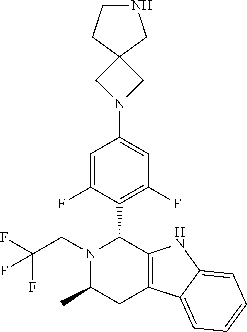 [SMILES: C[C@@H]1Cc2c([nH]c3ccccc23)[C@@H](c2c(F)cc(N3CC4(CCNC4)C3)cc2F)N1CC(F)(F)F] | 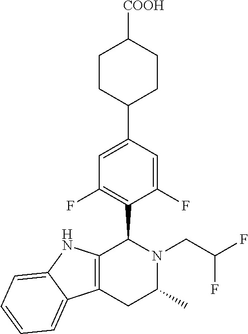 [SMILES: C[C@@H]1Cc2c([nH]c3ccccc23)[C@@H](c2c(F)cc(C3CCC(C(=O)O)CC3)cc2F)N1CC(F)F] |
| 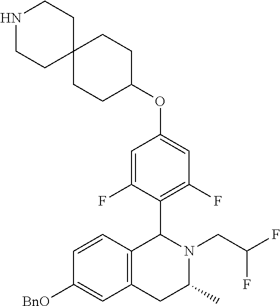 [SMILES: C[C@@H]1Cc2cc(OCc3ccccc3)ccc2C(c2c(F)cc(OC3CCC4(CCNCC4)CC3)cc2F)N1CC(F)F] | 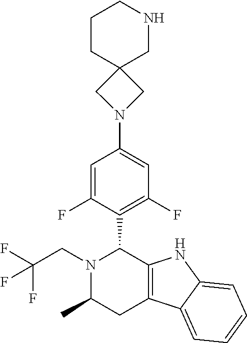 [SMILES: C[C@@H]1Cc2c([nH]c3ccccc23)[C@@H](c2c(F)cc(N3CC4(CCCNC4)C3)cc2F)N1CC(F)(F)F] | 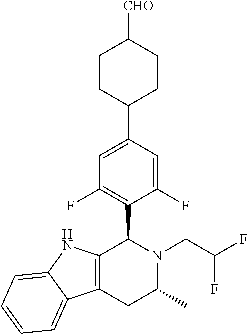 [SMILES: C[C@@H]1Cc2c([nH]c3ccccc23)[C@@H](c2c(F)cc(C3CCC(C=O)CC3)cc2F)N1CC(F)F] |
| 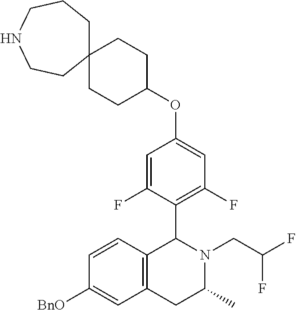 [SMILES: C[C@@H]1Cc2cc(OCc3ccccc3)ccc2C(c2c(F)cc(OC3CCC4(CCCNCC4)CC3)cc2F)N1CC(F)F] | 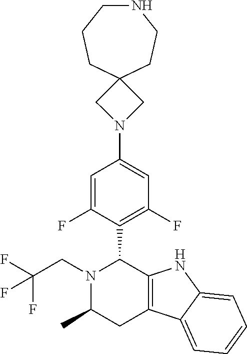 [SMILES: C[C@@H]1Cc2c([nH]c3ccccc23)[C@@H](c2c(F)cc(N3CC4(CCCNCC4)C3)cc2F)N1CC(F)(F)F] | 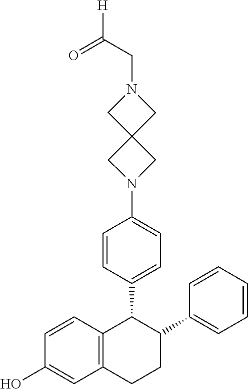 |
| 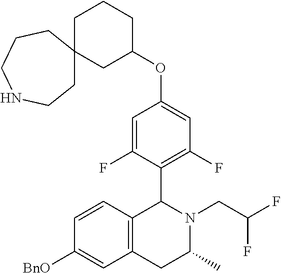 [SMILES: C[C@@H]1Cc2cc(OCc3ccccc3)ccc2C(c2c(F)cc(OC3CCCC4(CCCNCC4)C3)cc2F)N1CC(F)F] | 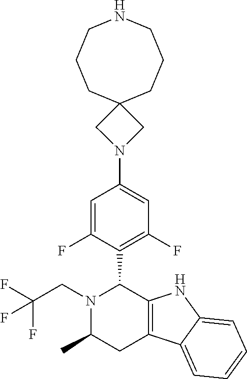 [SMILES: C[C@@H]1Cc2c([nH]c3ccccc23)[C@@H](c2c(F)cc(N3CC4(CCCNCCC4)C3)cc2F)N1CC(F)(F)F] | 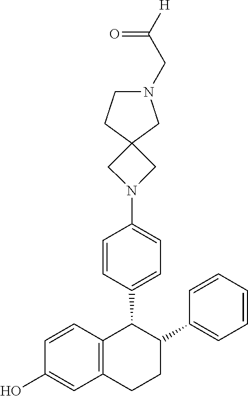 |
| 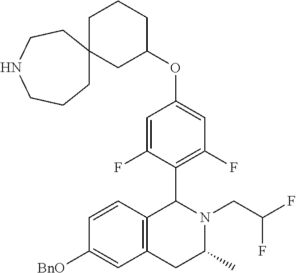 [SMILES: C[C@@H]1Cc2cc(OCc3ccccc3)ccc2C(c2c(F)cc(OC3CCCC4(CCCNCC4)C3)cc2F)N1CC(F)F] | 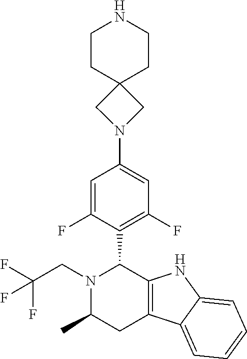 [SMILES: C[C@@H]1Cc2c([nH]c3ccccc23)[C@@H](c2c(F)cc(N3CC4(CCNCC4)C3)cc2F)N1CC(F)(F)F] | 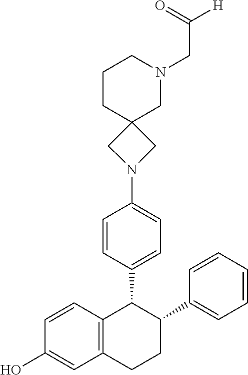 [SMILES: O=CCN1CCCC2(C1)CN(c1ccc([C@H]3c4ccc(O)cc4CC[C@H]3c3ccccc3)cc1)C2] |
| 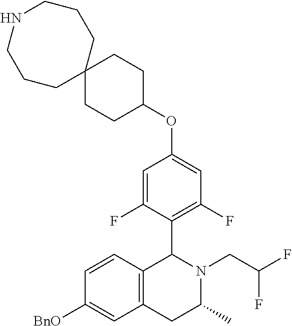 [SMILES: C[C@@H]1Cc2cc(OCc3ccccc3)ccc2C(c2c(F)cc(OC3CCC4(CCCNCCC4)CC3)cc2F)N1CC(F)F] | 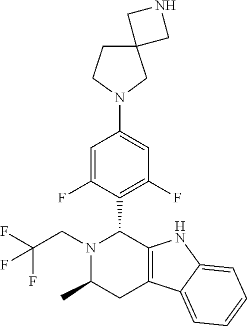 [SMILES: C[C@@H]1Cc2c([nH]c3ccccc23)[C@@H](c2c(F)cc(N3CCC4(CNC4)C3)cc2F)N1CC(F)(F)F] | 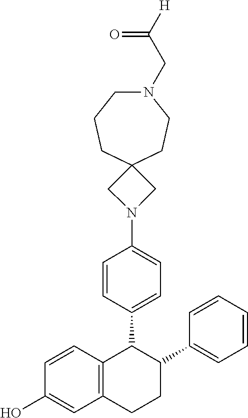 |
| 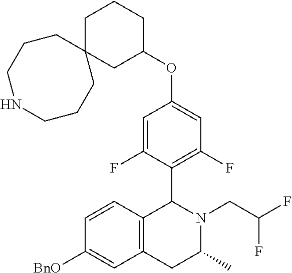 [SMILES: C[C@@H]1Cc2cc(OCc3ccccc3)ccc2C(c2c(F)cc(OC3CCCC4(CCCNCCC4)C3)cc2F)N1CC(F)F] | 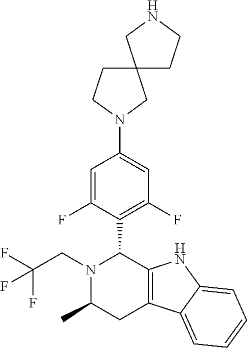 [SMILES: C[C@@H]1Cc2c([nH]c3ccccc23)[C@@H](c2c(F)cc(N3CCC4(CCNC4)C3)cc2F)N1CC(F)(F)F] | 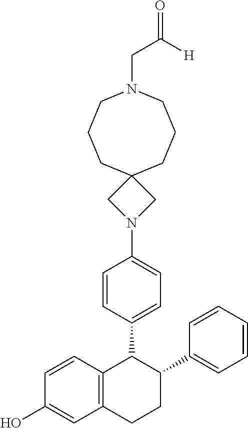 |
| 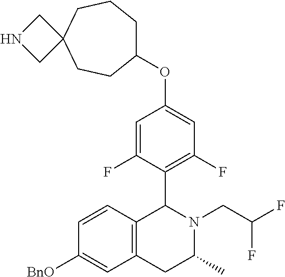 [SMILES: C[C@@H]1Cc2cc(OCc3ccccc3)ccc2C(c2c(F)cc(OC3CCCC4(CC3)CNC4)cc2F)N1CC(F)F] | 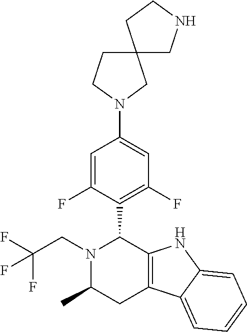 [SMILES: C[C@@H]1Cc2c([nH]c3ccccc23)[C@@H](c2c(F)cc(N3CCC4(CCNC4)C3)cc2F)N1CC(F)(F)F] | 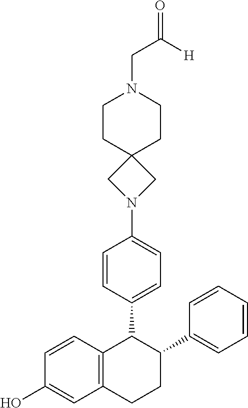 |
| 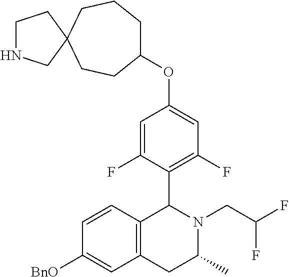 [SMILES: C[C@@H]1Cc2cc(OCc3ccccc3)ccc2C(c2c(F)cc(OC3CCCC4(CCNC4)CC3)cc2F)N1CC(F)F] | 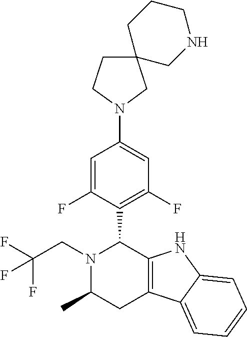 [SMILES: C[C@@H]1Cc2c([nH]c3ccccc23)[C@@H](c2c(F)cc(N3CCC4(CCCNC4)C3)cc2F)N1CC(F)(F)F] | 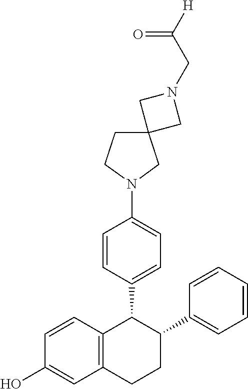 [SMILES: O=CCN1CC2(CCN(c3ccc([C@H]4c5ccc(O)cc5CC[C@H]4c4ccccc4)cc3)C2)C1] |
| 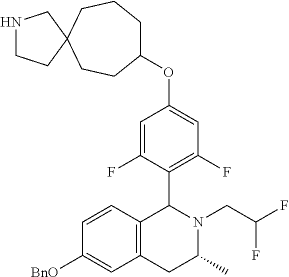 [SMILES: C[C@@H]1Cc2cc(OCc3ccccc3)ccc2C(c2c(F)cc(OC3CCCC4(CCNC4)CC3)cc2F)N1CC(F)F] | 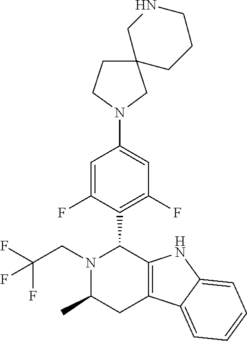 [SMILES: C[C@@H]1Cc2c([nH]c3ccccc23)[C@@H](c2c(F)cc(N3CCC4(CCCNC4)C3)cc2F)N1CC(F)(F)F] | 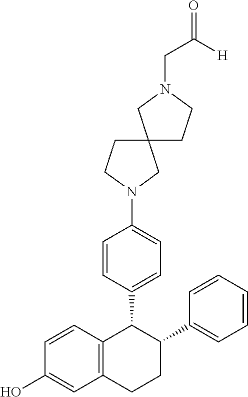 [SMILES: O=CCN1CCC2(CCN(c3ccc([C@H]4c5ccc(O)cc5CC[C@H]4c4ccccc4)cc3)C2)C1] |
| 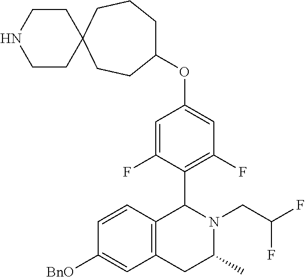 [SMILES: C[C@@H]1Cc2cc(OCc3ccccc3)ccc2C(c2c(F)cc(OC3CCCC4(CCNCC4)CC3)cc2F)N1CC(F)F] | 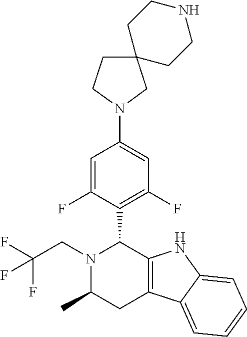 [SMILES: C[C@@H]1Cc2c([nH]c3ccccc23)[C@@H](c2c(F)cc(N3CCC4(CCNCC4)C3)cc2F)N1CC(F)(F)F] | 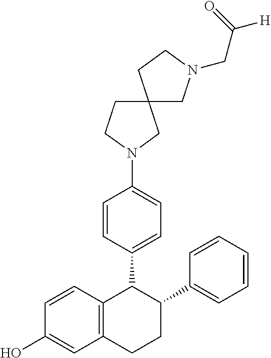 |
| 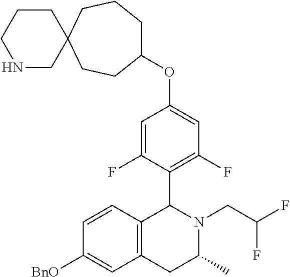 [SMILES: C[C@@H]1Cc2cc(OCc3ccccc3)ccc2C(c2c(F)cc(OC3CCCC4(CCCNC4)CC3)cc2F)N1CC(F)F] | 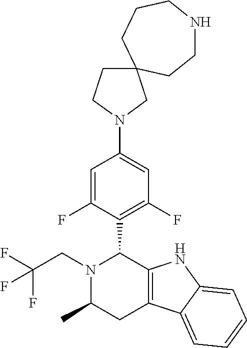 [SMILES: C[C@@H]1Cc2c([nH]c3ccccc23)[C@@H](c2c(F)cc(N3CCC4(CCCNCC4)C3)cc2F)N1CC(F)(F)F] | 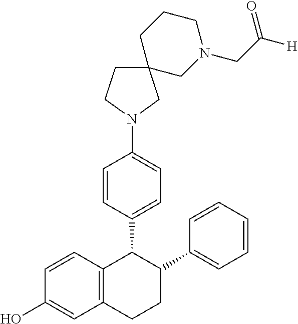 |
| 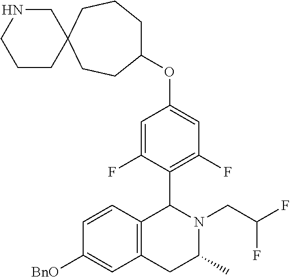 [SMILES: C[C@@H]1Cc2cc(OCc3ccccc3)ccc2C(c2c(F)cc(OC3CCCC4(CCCNC4)CC3)cc2F)N1CC(F)F] | 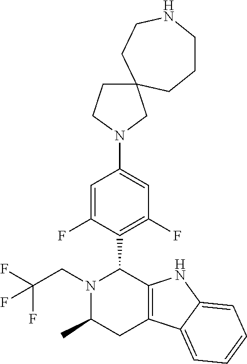 [SMILES: C[C@@H]1Cc2c([nH]c3ccccc23)[C@@H](c2c(F)cc(N3CCC4(CCCNCC4)C3)cc2F)N1CC(F)(F)F] | 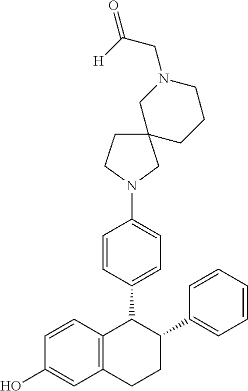 |
| 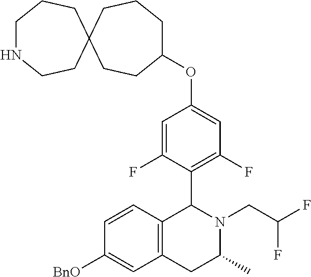 [SMILES: C[C@@H]1Cc2cc(OCc3ccccc3)ccc2C(c2c(F)cc(OC3CCCC4(CCCNCC4)CC3)cc2F)N1CC(F)F] | 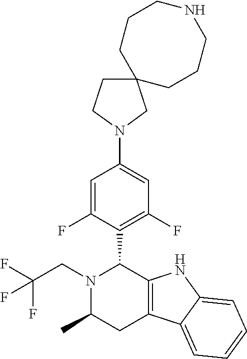 [SMILES: C[C@@H]1Cc2c([nH]c3ccccc23)[C@@H](c2c(F)cc(N3CCC4(CCCNCCC4)C3)cc2F)N1CC(F)(F)F] | 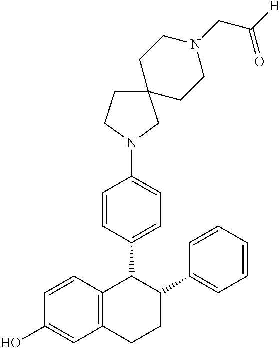 |
| 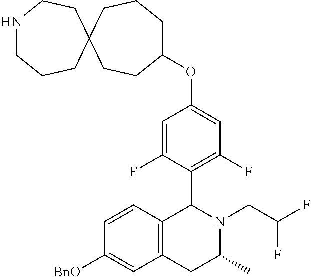 [SMILES: C[C@@H]1Cc2cc(OCc3ccccc3)ccc2C(c2c(F)cc(OC3CCCC4(CCCNCC4)CC3)cc2F)N1CC(F)F] | 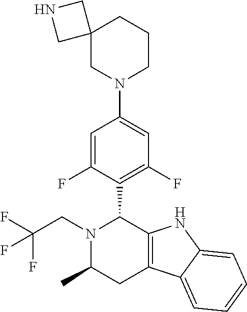 [SMILES: C[C@@H]1Cc2c([nH]c3ccccc23)[C@@H](c2c(F)cc(N3CCCC4(CNC4)C3)cc2F)N1CC(F)(F)F] | 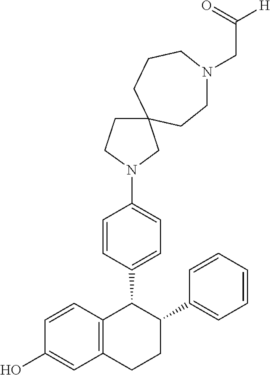 [SMILES: O=CCN1CCCC2(CC1)CCN(c1ccc([C@H]3c4ccc(O)cc4CC[C@H]3c3ccccc3)cc1)C2] |
| 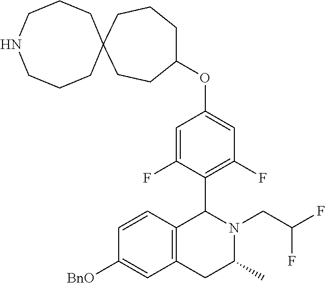 [SMILES: C[C@@H]1Cc2cc(OCc3ccccc3)ccc2C(c2c(F)cc(OC3CCCC4(CCCNCCC4)CC3)cc2F)N1CC(F)F] | 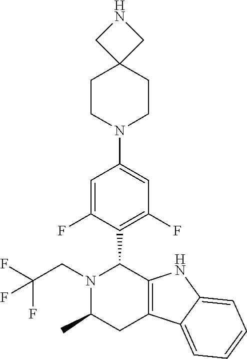 [SMILES: C[C@@H]1Cc2c([nH]c3ccccc23)[C@@H](c2c(F)cc(N3CCC4(CC3)CNC4)cc2F)N1CC(F)(F)F] | 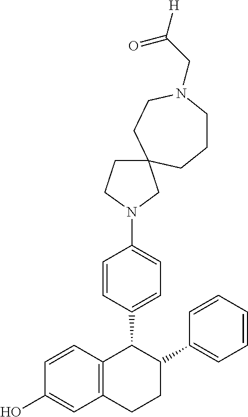 |
| 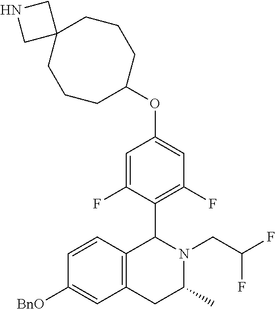 [SMILES: C[C@@H]1Cc2cc(OCc3ccccc3)ccc2C(c2c(F)cc(OC3CCCC4(CCC3)CNC4)cc2F)N1CC(F)F] | 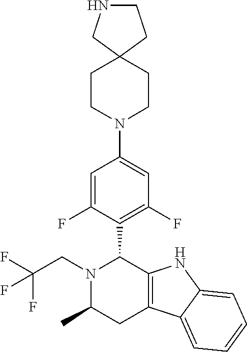 [SMILES: C[C@@H]1Cc2c([nH]c3ccccc23)[C@@H](c2c(F)cc(N3CCC4(CCNC4)CC3)cc2F)N1CC(F)(F)F] | 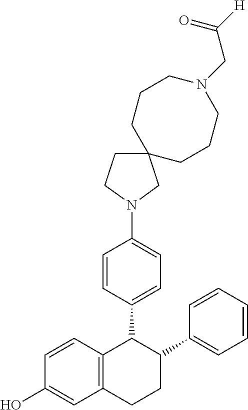 |
| 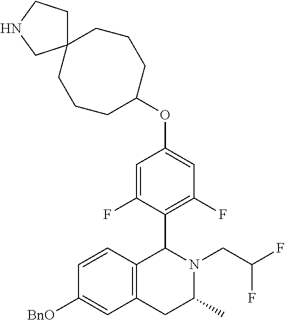 [SMILES: C[C@@H]1Cc2cc(OCc3ccccc3)ccc2C(c2c(F)cc(OC3CCCC4(CCC3)CCNC4)cc2F)N1CC(F)F] | 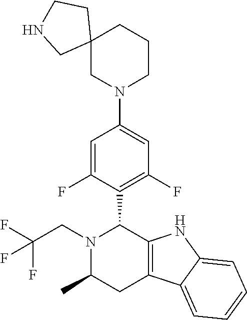 [SMILES: C[C@@H]1Cc2c([nH]c3ccccc23)[C@@H](c2c(F)cc(N3CCCC4(CCNC4)C3)cc2F)N1CC(F)(F)F] | 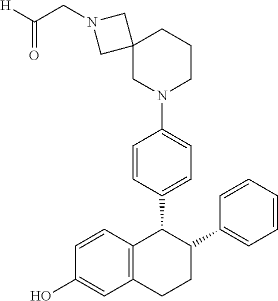 |
| 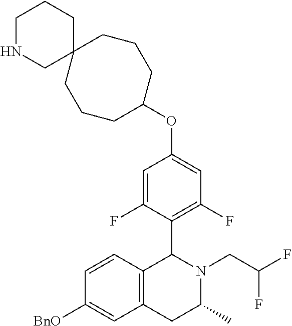 [SMILES: C[C@@H]1Cc2cc(OCc3ccccc3)ccc2C(c2c(F)cc(OC3CCCC4(CCCNC4)CCC3)cc2F)N1CC(F)F] | 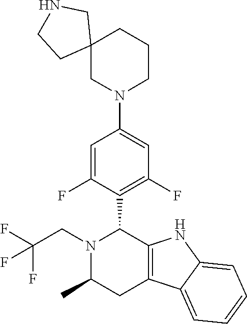 [SMILES: C[C@@H]1Cc2c([nH]c3ccccc23)[C@@H](c2c(F)cc(N3CCCC4(CCNC4)C3)cc2F)N1CC(F)(F)F] | 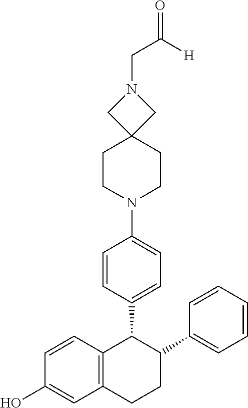 |
| 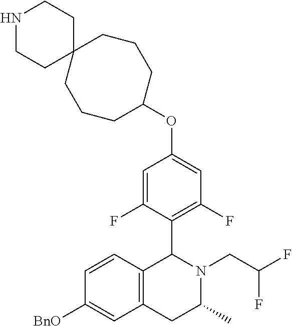 [SMILES: C[C@@H]1Cc2cc(OCc3ccccc3)ccc2C(c2c(F)cc(OC3CCCC4(CCC3)CCNCC4)cc2F)N1CC(F)F] | 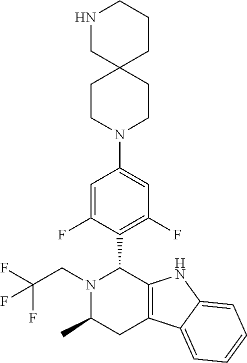 [SMILES: C[C@@H]1Cc2c([nH]c3ccccc23)[C@@H](c2c(F)cc(N3CCC4(CCCNC4)CC3)cc2F)N1CC(F)(F)F] | 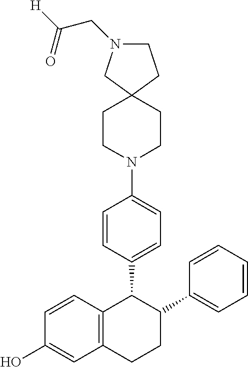 |
| 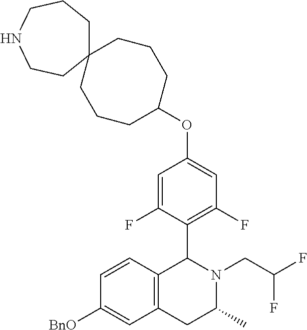 [SMILES: C[C@@H]1Cc2cc(OCc3ccccc3)ccc2C(c2c(F)cc(OC3CCCC4(CCCNCC4)CCC3)cc2F)N1CC(F)F] | 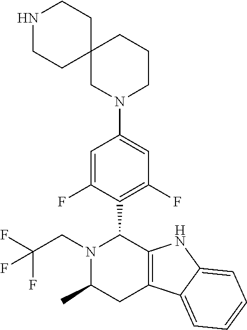 [SMILES: C[C@@H]1Cc2c([nH]c3ccccc23)[C@@H](c2c(F)cc(N3CCCC4(CCNCC4)C3)cc2F)N1CC(F)(F)F] | 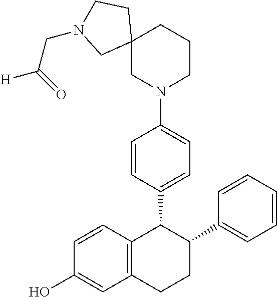 [SMILES: O=CCN1CCC2(CCCN(c3ccc([C@H]4c5ccc(O)cc5CC[C@H]4c4ccccc4)cc3)C2)C1] |
| 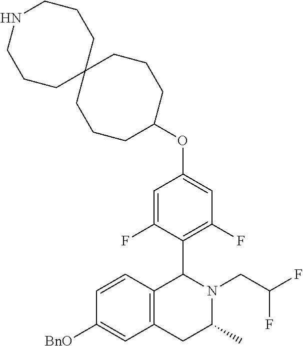 [SMILES: C[C@@H]1Cc2cc(OCc3ccccc3)ccc2C(c2c(F)cc(OC3CCCC4(CCCNCCC4)CCC3)cc2F)N1CC(F)F] | 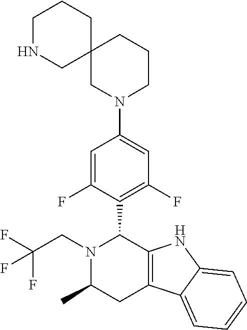 [SMILES: C[C@@H]1Cc2c([nH]c3ccccc23)[C@@H](c2c(F)cc(N3CCCC4(CCCNC4)C3)cc2F)N1CC(F)(F)F] | 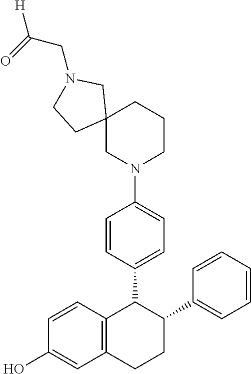 |
| 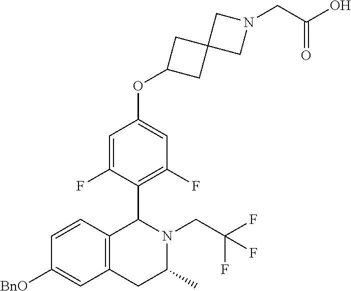 [SMILES: C[C@@H]1Cc2cc(OCc3ccccc3)ccc2C(c2c(F)cc(OC3CC4(C3)CN(CC(=O)O)C4)cc2F)N1CC(F)(F)F] | 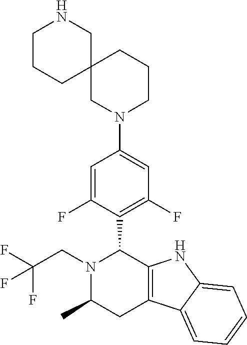 [SMILES: C[C@@H]1Cc2c([nH]c3ccccc23)[C@@H](c2c(F)cc(N3CCCC4(CCCNC4)C3)cc2F)N1CC(F)(F)F] | 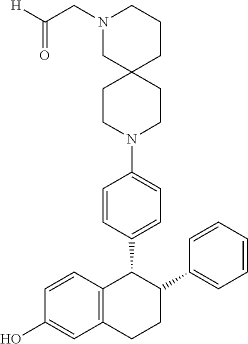 |
| 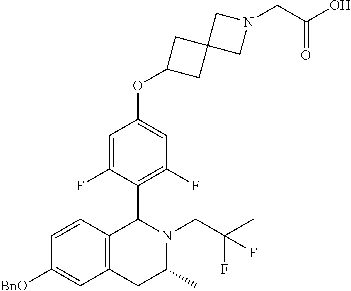 [SMILES: C[C@@H]1Cc2cc(OCc3ccccc3)ccc2C(c2c(F)cc(OC3CC4(C3)CN(CC(=O)O)C4)cc2F)N1CC(C)(F)F] | 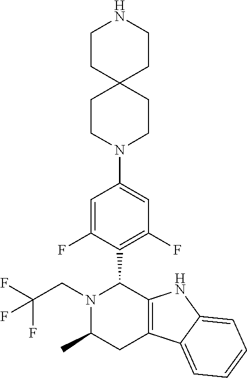 [SMILES: C[C@@H]1Cc2c([nH]c3ccccc23)[C@@H](c2c(F)cc(N3CCC4(CCNCC4)CC3)cc2F)N1CC(F)(F)F] | 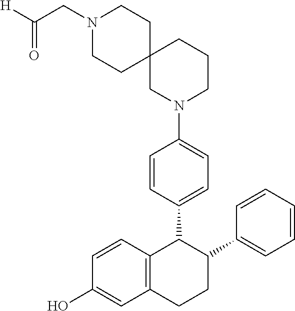 |
| 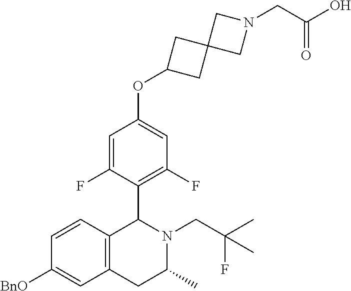 [SMILES: C[C@@H]1Cc2cc(OCc3ccccc3)ccc2C(c2c(F)cc(OC3CC4(C3)CN(CC(=O)O)C4)cc2F)N1CC(C)(C)F] | 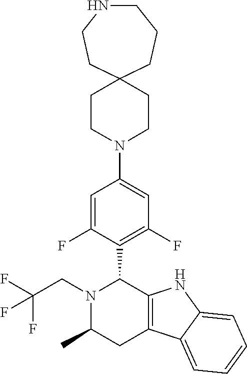 [SMILES: C[C@@H]1Cc2c([nH]c3ccccc23)[C@@H](c2c(F)cc(N3CCC4(CCCNCC4)CC3)cc2F)N1CC(F)(F)F] | 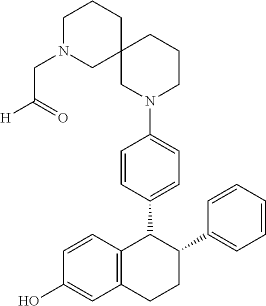 |
| 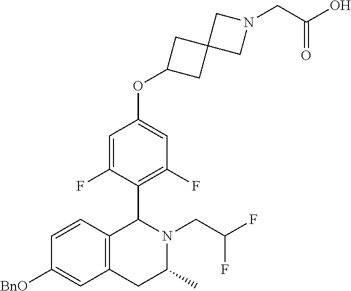 [SMILES: C[C@@H]1Cc2cc(OCc3ccccc3)ccc2C(c2c(F)cc(OC3CC4(C3)CN(CC(=O)O)C4)cc2F)N1CC(F)F] | 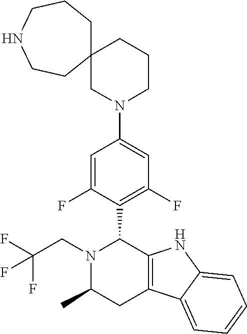 [SMILES: C[C@@H]1Cc2c([nH]c3ccccc23)[C@@H](c2c(F)cc(N3CCCC4(CCCNCC4)C3)cc2F)N1CC(F)(F)F] | 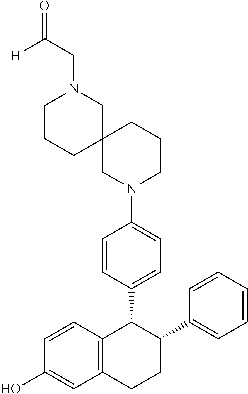 [SMILES: O=CCN1CCCC2(CCCN(c3ccc([C@H]4c5ccc(O)cc5CC[C@H]4c4ccccc4)cc3)C2)C1] |
| 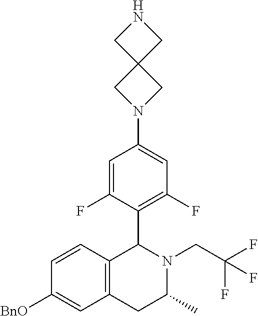 [SMILES: C[C@@H]1Cc2cc(OCc3ccccc3)ccc2C(c2c(F)cc(N3CC4(CNC4)C3)cc2F)N1CC(F)(F)F] | 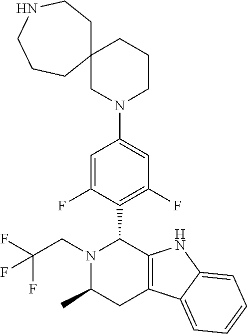 [SMILES: C[C@@H]1Cc2c([nH]c3ccccc23)[C@@H](c2c(F)cc(N3CCCC4(CCCNCC4)C3)cc2F)N1CC(F)(F)F] | 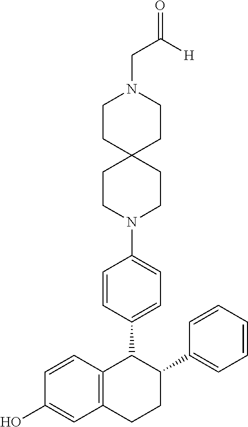 |
| 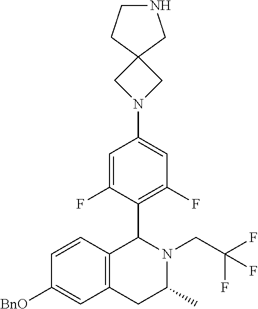 [SMILES: C[C@@H]1Cc2cc(OCc3ccccc3)ccc2C(c2c(F)cc(N3CC4(CCNC4)C3)cc2F)N1CC(F)(F)F] | 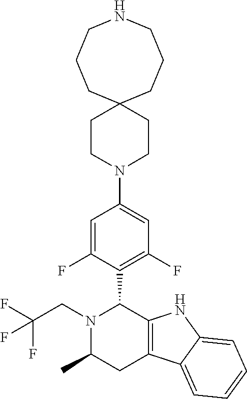 [SMILES: C[C@@H]1Cc2c([nH]c3ccccc23)[C@@H](c2c(F)cc(N3CCC4(CCCNCCC4)CC3)cc2F)N1CC(F)(F)F] | 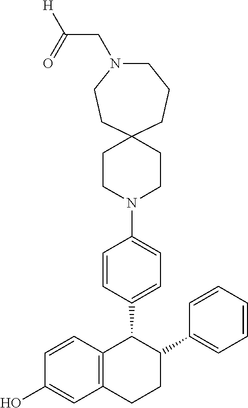 |
| 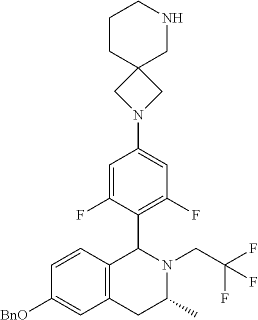 [SMILES: C[C@@H]1Cc2cc(OCc3ccccc3)ccc2C(c2c(F)cc(N3CC4(CCCNC4)C3)cc2F)N1CC(F)(F)F] | 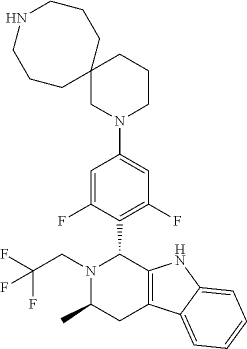 [SMILES: C[C@@H]1Cc2c([nH]c3ccccc23)[C@@H](c2c(F)cc(N3CCCC4(CCCNCCC4)C3)cc2F)N1CC(F)(F)F] | 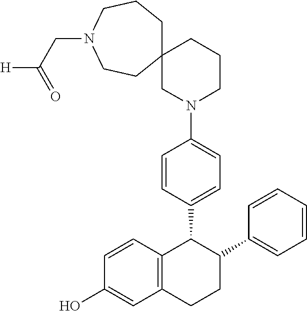 |
| 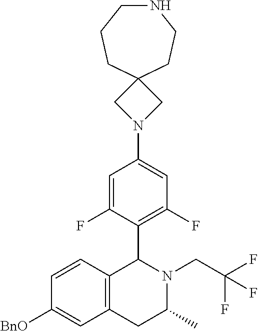 [SMILES: C[C@@H]1Cc2cc(OCc3ccccc3)ccc2C(c2c(F)cc(N3CC4(CCCNCC4)C3)cc2F)N1CC(F)(F)F] | 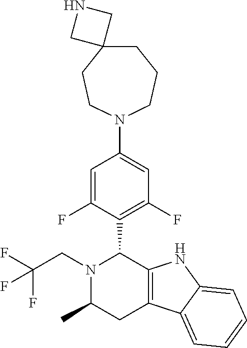 [SMILES: C[C@@H]1Cc2c([nH]c3ccccc23)[C@@H](c2c(F)cc(N3CCCC4(CC3)CNC4)cc2F)N1CC(F)(F)F] | 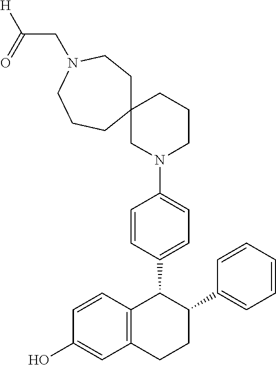 [SMILES: O=CCN1CCCC2(CCCN(c3ccc([C@H]4c5ccc(O)cc5CC[C@H]4c4ccccc4)cc3)C2)CC1] |
| 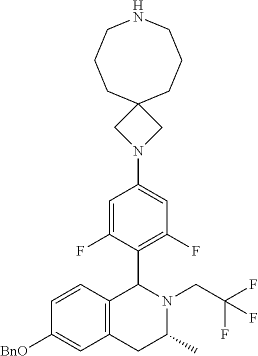 [SMILES: C[C@@H]1Cc2cc(OCc3ccccc3)ccc2C(c2c(F)cc(N3CC4(CCCNCCC4)C3)cc2F)N1CC(F)(F)F] | 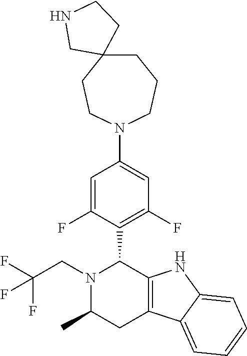 [SMILES: C[C@@H]1Cc2c([nH]c3ccccc23)[C@@H](c2c(F)cc(N3CCCC4(CCNC4)CC3)cc2F)N1CC(F)(F)F] | 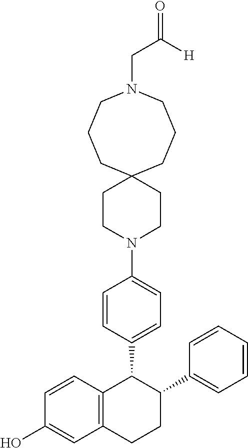 [SMILES: O=CCN1CCCC2(CCC1)CCN(c1ccc([C@H]3c4ccc(O)cc4CC[C@H]3c3ccccc3)cc1)CC2] |
| 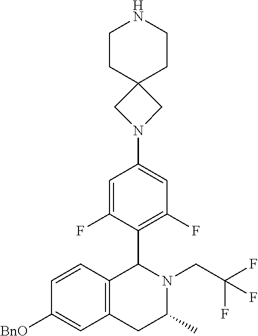 [SMILES: C[C@@H]1Cc2cc(OCc3ccccc3)ccc2C(c2c(F)cc(N3CC4(CCNCC4)C3)cc2F)N1CC(F)(F)F] | 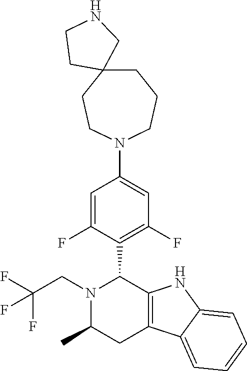 [SMILES: C[C@@H]1Cc2c([nH]c3ccccc23)[C@@H](c2c(F)cc(N3CCCC4(CCNC4)CC3)cc2F)N1CC(F)(F)F] | 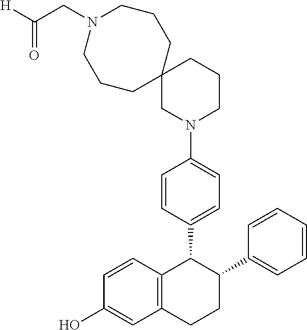 [SMILES: O=CCN1CCCC2(CCC1)CCCN(c1ccc([C@H]3c4ccc(O)cc4CC[C@H]3c3ccccc3)cc1)C2] |
| 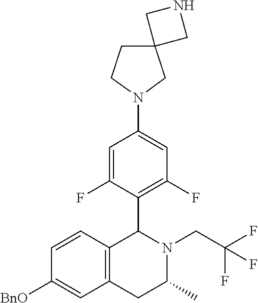 [SMILES: C[C@@H]1Cc2cc(OCc3ccccc3)ccc2C(c2c(F)cc(N3CCC4(CNC4)C3)cc2F)N1CC(F)(F)F] | 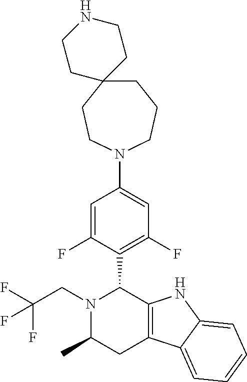 [SMILES: C[C@@H]1Cc2c([nH]c3ccccc23)[C@@H](c2c(F)cc(N3CCCC4(CCNCC4)CC3)cc2F)N1CC(F)(F)F] | 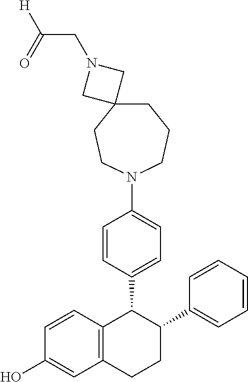 [SMILES: O=CCN1CC2(CCCN(c3ccc([C@H]4c5ccc(O)cc5CC[C@H]4c4ccccc4)cc3)CC2)C1] |
| 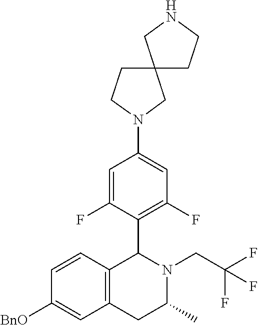 [SMILES: C[C@@H]1Cc2cc(OCc3ccccc3)ccc2C(c2c(F)cc(N3CCC4(CCNC4)C3)cc2F)N1CC(F)(F)F] | 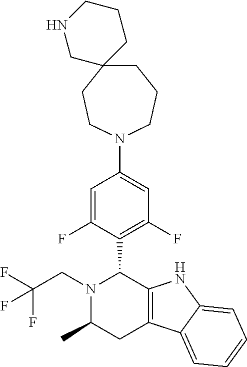 [SMILES: C[C@@H]1Cc2c([nH]c3ccccc23)[C@@H](c2c(F)cc(N3CCCC4(CCCNC4)CC3)cc2F)N1CC(F)(F)F] | 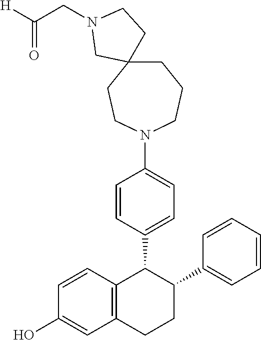 [SMILES: O=CCN1CCC2(CCCN(c3ccc([C@H]4c5ccc(O)cc5CC[C@H]4c4ccccc4)cc3)CC2)C1] |
| 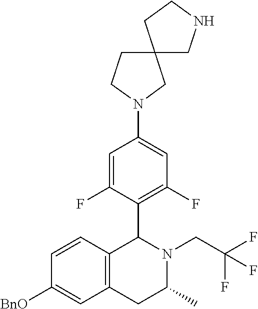 [SMILES: C[C@@H]1Cc2cc(OCc3ccccc3)ccc2C(c2c(F)cc(N3CCC4(CCNC4)C3)cc2F)N1CC(F)(F)F] | 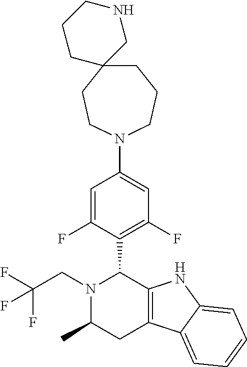 [SMILES: C[C@@H]1Cc2c([nH]c3ccccc23)[C@@H](c2c(F)cc(N3CCCC4(CCCNC4)CC3)cc2F)N1CC(F)(F)F] | 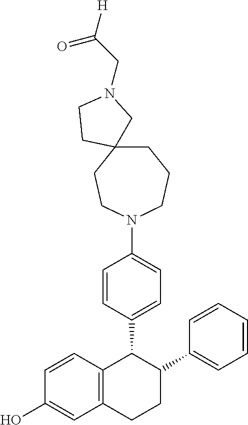 |
| 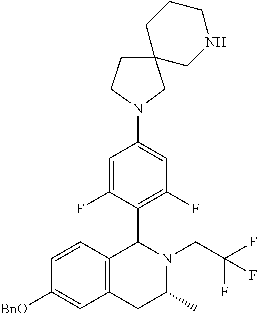 [SMILES: C[C@@H]1Cc2cc(OCc3ccccc3)ccc2C(c2c(F)cc(N3CCC4(CCCNC4)C3)cc2F)N1CC(F)(F)F] | 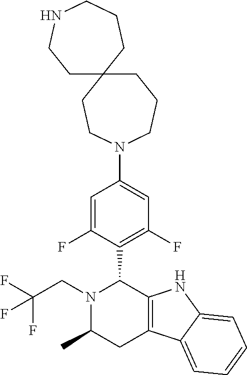 [SMILES: C[C@@H]1Cc2c([nH]c3ccccc23)[C@@H](c2c(F)cc(N3CCCC4(CCCNCC4)CC3)cc2F)N1CC(F)(F)F] | 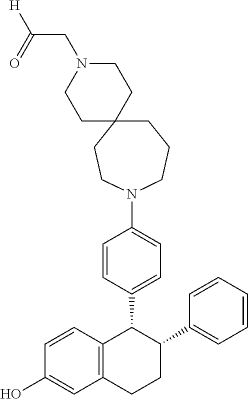 [SMILES: O=CCN1CCC2(CCCN(c3ccc([C@H]4c5ccc(O)cc5CC[C@H]4c4ccccc4)cc3)CC2)CC1] |
| 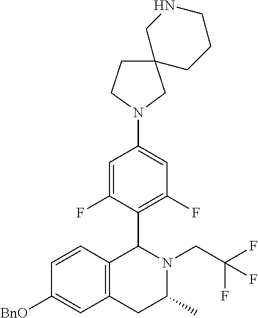 [SMILES: C[C@@H]1Cc2cc(OCc3ccccc3)ccc2C(c2c(F)cc(N3CCC4(CCCNC4)C3)cc2F)N1CC(F)(F)F] | 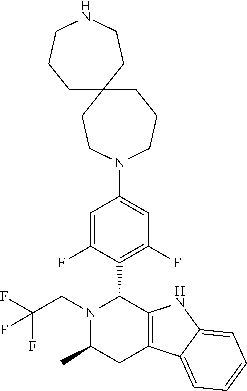 [SMILES: C[C@@H]1Cc2c([nH]c3ccccc23)[C@@H](c2c(F)cc(N3CCCC4(CCCNCC4)CC3)cc2F)N1CC(F)(F)F] | 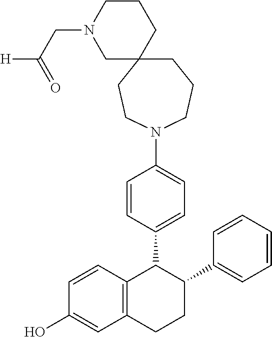 |
| 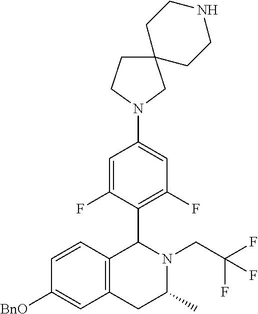 [SMILES: C[C@@H]1Cc2cc(OCc3ccccc3)ccc2C(c2c(F)cc(N3CCC4(CCNCC4)C3)cc2F)N1CC(F)(F)F] | 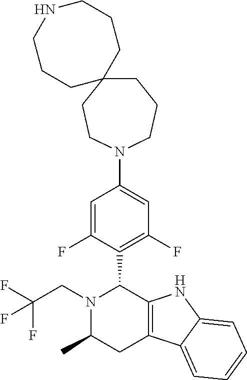 [SMILES: C[C@@H]1Cc2c([nH]c3ccccc23)[C@@H](c2c(F)cc(N3CCCC4(CCCNCCC4)CC3)cc2F)N1CC(F)(F)F] | 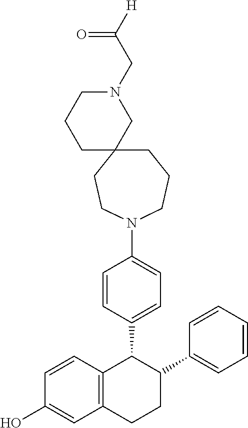 |
| 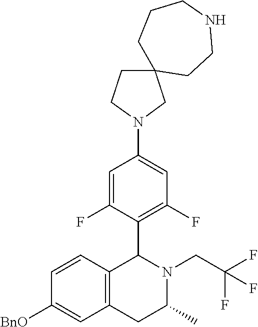 [SMILES: C[C@@H]1Cc2cc(OCc3ccccc3)ccc2C(c2c(F)cc(N3CCC4(CCCNCC4)C3)cc2F)N1CC(F)(F)F] | 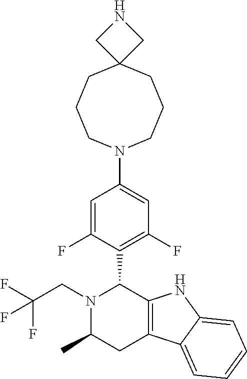 [SMILES: C[C@@H]1Cc2c([nH]c3ccccc23)[C@@H](c2c(F)cc(N3CCCC4(CCC3)CNC4)cc2F)N1CC(F)(F)F] | 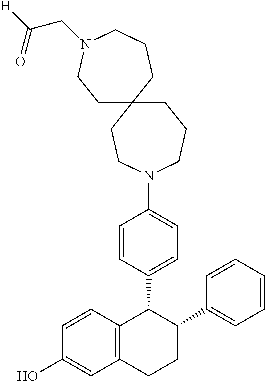 [SMILES: O=CCN1CCCC2(CCCN(c3ccc([C@H]4c5ccc(O)cc5CC[C@H]4c4ccccc4)cc3)CC2)CC1] |
| 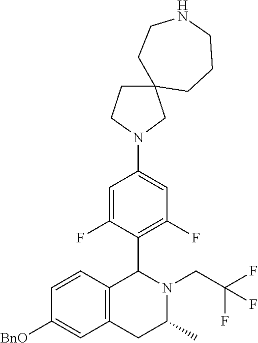 [SMILES: C[C@@H]1Cc2cc(OCc3ccccc3)ccc2C(c2c(F)cc(N3CCC4(CCCNCC4)C3)cc2F)N1CC(F)(F)F] | 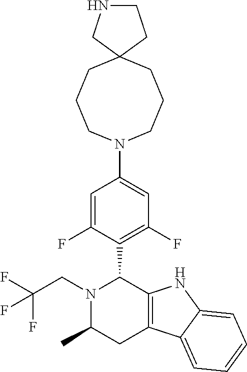 [SMILES: C[C@@H]1Cc2c([nH]c3ccccc23)[C@@H](c2c(F)cc(N3CCCC4(CCC3)CCNC4)cc2F)N1CC(F)(F)F] | 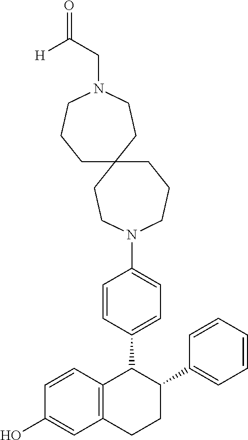 |
| 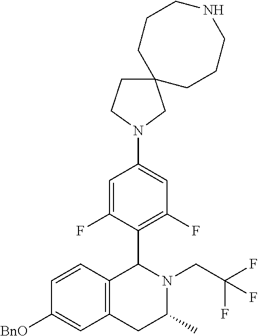 [SMILES: C[C@@H]1Cc2cc(OCc3ccccc3)ccc2C(c2c(F)cc(N3CCC4(CCCNCCC4)C3)cc2F)N1CC(F)(F)F] | 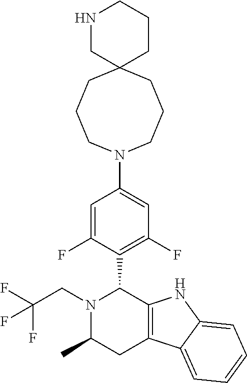 [SMILES: C[C@@H]1Cc2c([nH]c3ccccc23)[C@@H](c2c(F)cc(N3CCCC4(CCCNC4)CCC3)cc2F)N1CC(F)(F)F] | 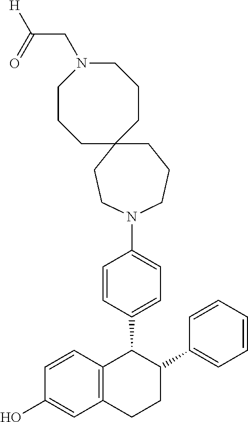 |
| 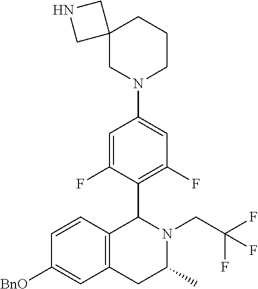 [SMILES: C[C@@H]1Cc2cc(OCc3ccccc3)ccc2C(c2c(F)cc(N3CCCC4(CNC4)C3)cc2F)N1CC(F)(F)F] | 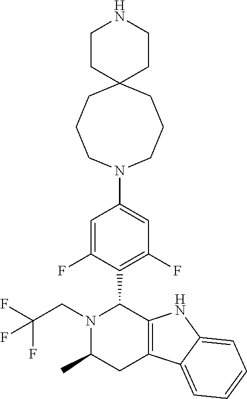 [SMILES: C[C@@H]1Cc2c([nH]c3ccccc23)[C@@H](c2c(F)cc(N3CCCC4(CCC3)CCNCC4)cc2F)N1CC(F)(F)F] | 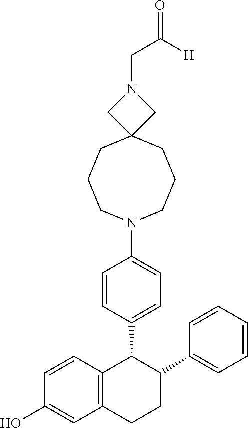 |
| 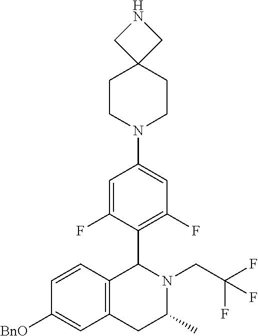 [SMILES: C[C@@H]1Cc2cc(OCc3ccccc3)ccc2C(c2c(F)cc(N3CCC4(CC3)CNC4)cc2F)N1CC(F)(F)F] | 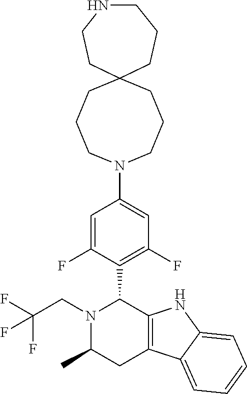 [SMILES: C[C@@H]1Cc2c([nH]c3ccccc23)[C@@H](c2c(F)cc(N3CCCC4(CCCNCC4)CCC3)cc2F)N1CC(F)(F)F] | 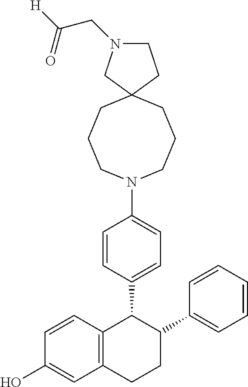 |
| 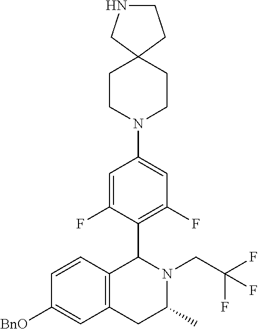 [SMILES: C[C@@H]1Cc2cc(OCc3ccccc3)ccc2C(c2c(F)cc(N3CCC4(CCNC4)CC3)cc2F)N1CC(F)(F)F] | 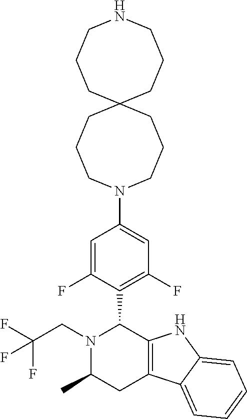 [SMILES: C[C@@H]1Cc2c([nH]c3ccccc23)[C@@H](c2c(F)cc(N3CCCC4(CCCNCCC4)CCC3)cc2F)N1CC(F)(F)F] | 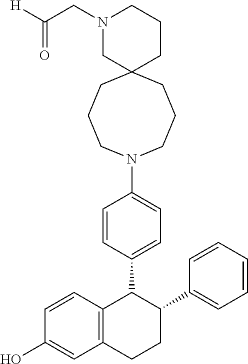 |
| 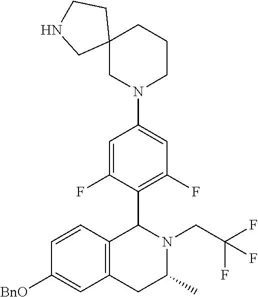 [SMILES: C[C@@H]1Cc2cc(OCc3ccccc3)ccc2C(c2c(F)cc(N3CCCC4(CCNC4)C3)cc2F)N1CC(F)(F)F] | 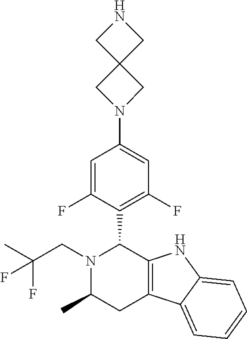 [SMILES: C[C@@H]1Cc2c([nH]c3ccccc23)[C@@H](c2c(F)cc(N3CC4(CNC4)C3)cc2F)N1CC(C)(F)F] | 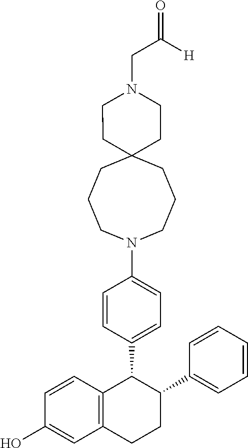 [SMILES: O=CCN1CCC2(CCCN(c3ccc([C@H]4c5ccc(O)cc5CC[C@H]4c4ccccc4)cc3)CCC2)CC1] |
| 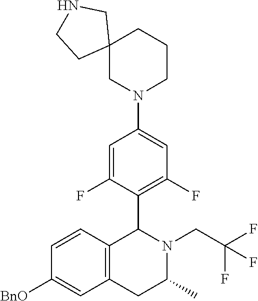 [SMILES: C[C@@H]1Cc2cc(OCc3ccccc3)ccc2C(c2c(F)cc(N3CCCC4(CCNC4)C3)cc2F)N1CC(F)(F)F] | 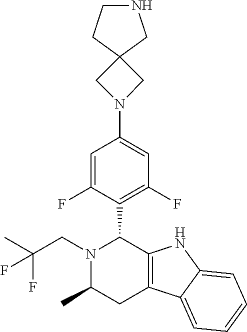 [SMILES: C[C@@H]1Cc2c([nH]c3ccccc23)[C@@H](c2c(F)cc(N3CC4(CCNC4)C3)cc2F)N1CC(C)(F)F] | 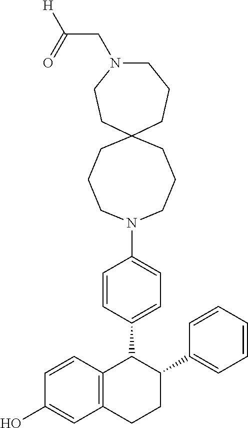 |
| 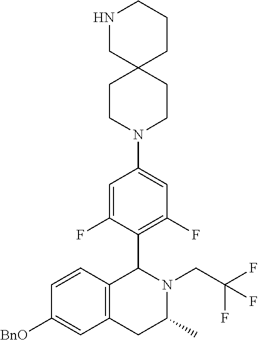 [SMILES: C[C@@H]1Cc2cc(OCc3ccccc3)ccc2C(c2c(F)cc(N3CCC4(CCCNC4)CC3)cc2F)N1CC(F)(F)F] | 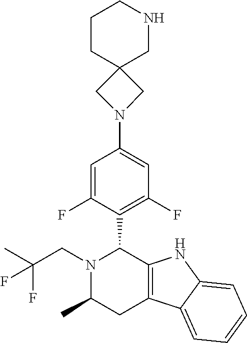 [SMILES: C[C@@H]1Cc2c([nH]c3ccccc23)[C@@H](c2c(F)cc(N3CC4(CCCNC4)C3)cc2F)N1CC(C)(F)F] | 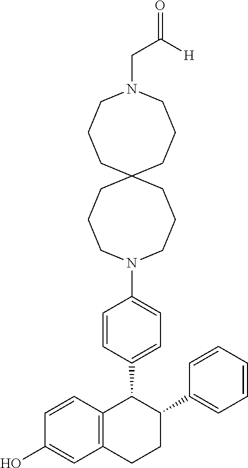 [SMILES: O=CCN1CCCC2(CCC1)CCCN(c1ccc([C@H]3c4ccc(O)cc4CC[C@H]3c3ccccc3)cc1)CCC2] |
| 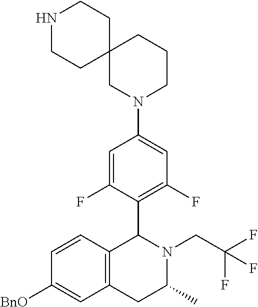 [SMILES: C[C@@H]1Cc2cc(OCc3ccccc3)ccc2C(c2c(F)cc(N3CCCC4(CCNCC4)C3)cc2F)N1CC(F)(F)F] | 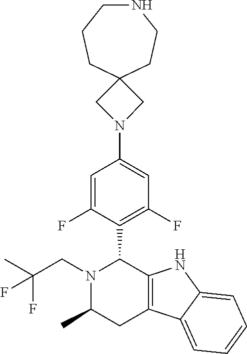 [SMILES: C[C@@H]1Cc2c([nH]c3ccccc23)[C@@H](c2c(F)cc(N3CC4(CCCNCC4)C3)cc2F)N1CC(C)(F)F] | 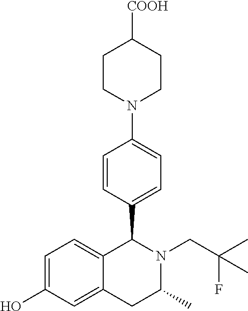 [SMILES: C[C@@H]1Cc2cc(O)ccc2[C@@H](c2ccc(N3CCC(C(=O)O)CC3)cc2)N1CC(C)(C)F] |
| 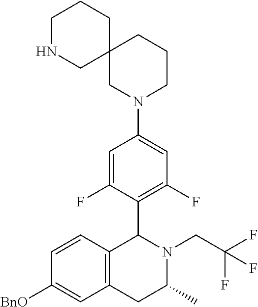 [SMILES: C[C@@H]1Cc2cc(OCc3ccccc3)ccc2C(c2c(F)cc(N3CCCC4(CCCNC4)C3)cc2F)N1CC(F)(F)F] | 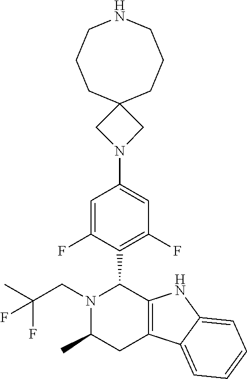 [SMILES: C[C@@H]1Cc2c([nH]c3ccccc23)[C@@H](c2c(F)cc(N3CC4(CCCNCCC4)C3)cc2F)N1CC(C)(F)F] | 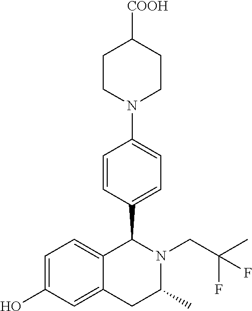 [SMILES: C[C@@H]1Cc2cc(O)ccc2[C@@H](c2ccc(N3CCC(C(=O)O)CC3)cc2)N1CC(C)(F)F] |
| 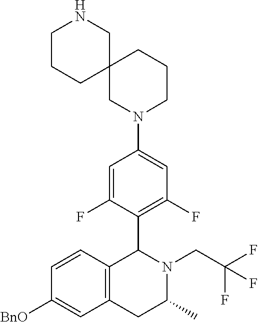 [SMILES: C[C@@H]1Cc2cc(OCc3ccccc3)ccc2C(c2c(F)cc(N3CCCC4(CCCNC4)C3)cc2F)N1CC(F)(F)F] | 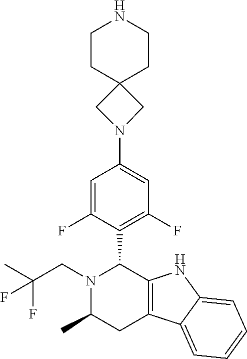 [SMILES: C[C@@H]1Cc2c([nH]c3ccccc23)[C@@H](c2c(F)cc(N3CC4(CCNCC4)C3)cc2F)N1CC(C)(F)F] | 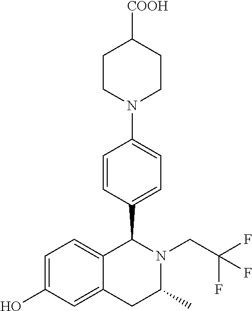 [SMILES: C[C@@H]1Cc2cc(O)ccc2[C@@H](c2ccc(N3CCC(C(=O)O)CC3)cc2)N1CC(F)(F)F] |
| 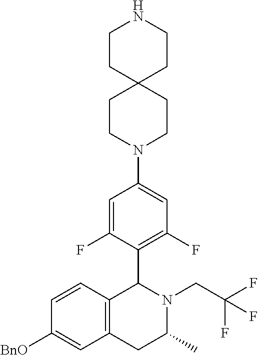 [SMILES: C[C@@H]1Cc2cc(OCc3ccccc3)ccc2C(c2c(F)cc(N3CCC4(CCNCC4)CC3)cc2F)N1CC(F)(F)F] | 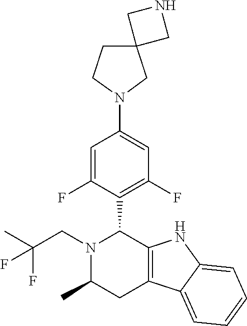 [SMILES: C[C@@H]1Cc2c([nH]c3ccccc23)[C@@H](c2c(F)cc(N3CCC4(CNC4)C3)cc2F)N1CC(C)(F)F] | 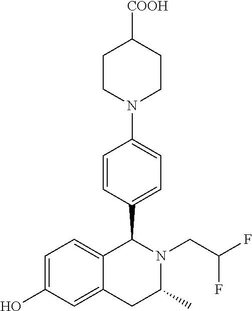 [SMILES: C[C@@H]1Cc2cc(O)ccc2[C@@H](c2ccc(N3CCC(C(=O)O)CC3)cc2)N1CC(F)F] |
| 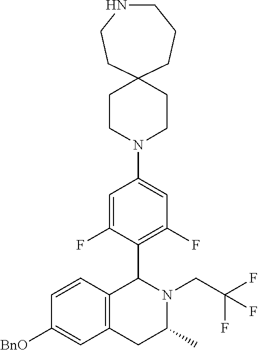 [SMILES: C[C@@H]1Cc2cc(OCc3ccccc3)ccc2C(c2c(F)cc(N3CCC4(CCCNCC4)CC3)cc2F)N1CC(F)(F)F] | 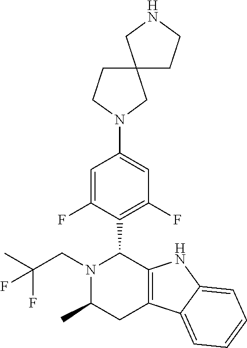 [SMILES: C[C@@H]1Cc2c([nH]c3ccccc23)[C@@H](c2c(F)cc(N3CCC4(CCNC4)C3)cc2F)N1CC(C)(F)F] | 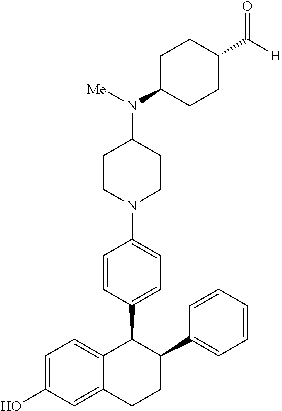 [SMILES: CN(C1CCN(c2ccc([C@@H]3c4ccc(O)cc4CC[C@@H]3c3ccccc3)cc2)CC1)[C@H]1CC[C@H](C=O)CC1] |
| 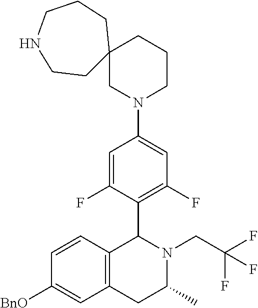 [SMILES: C[C@@H]1Cc2cc(OCc3ccccc3)ccc2C(c2c(F)cc(N3CCCC4(CCCNCC4)C3)cc2F)N1CC(F)(F)F] | 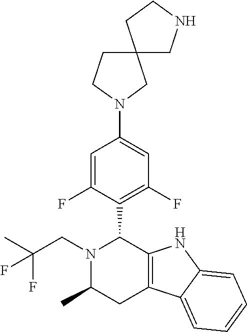 [SMILES: C[C@@H]1Cc2c([nH]c3ccccc23)[C@@H](c2c(F)cc(N3CCC4(CCNC4)C3)cc2F)N1CC(C)(F)F] | 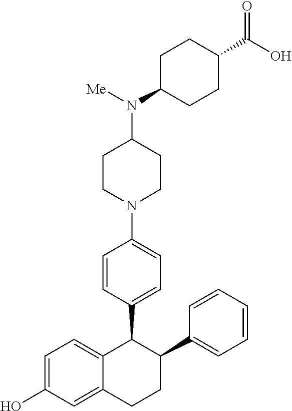 [SMILES: CN(C1CCN(c2ccc([C@@H]3c4ccc(O)cc4CC[C@@H]3c3ccccc3)cc2)CC1)[C@H]1CC[C@H](C(=O)O)CC1] |
| 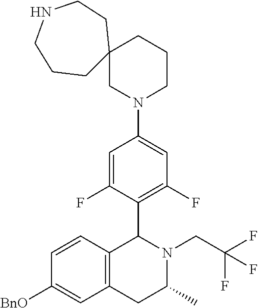 [SMILES: C[C@@H]1Cc2cc(OCc3ccccc3)ccc2C(c2c(F)cc(N3CCCC4(CCCNCC4)C3)cc2F)N1CC(F)(F)F] | 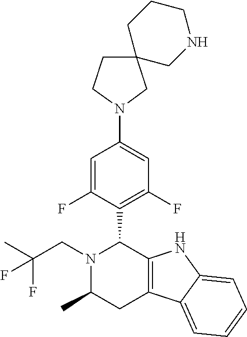 [SMILES: C[C@@H]1Cc2c([nH]c3ccccc23)[C@@H](c2c(F)cc(N3CCC4(CCCNC4)C3)cc2F)N1CC(C)(F)F] | 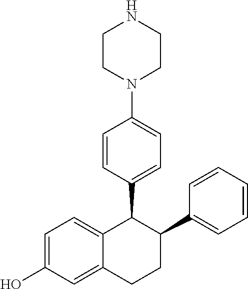 [SMILES: Oc1ccc2c(c1)CC[C@H](c1ccccc1)[C@@H]2c1ccc(N2CCNCC2)cc1] |
| 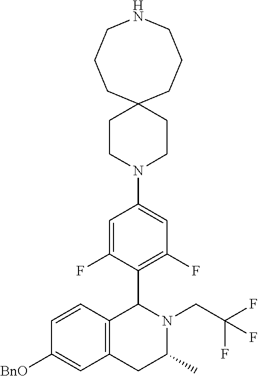 [SMILES: C[C@@H]1Cc2cc(OCc3ccccc3)ccc2C(c2c(F)cc(N3CCC4(CCCNCCC4)CC3)cc2F)N1CC(F)(F)F] | 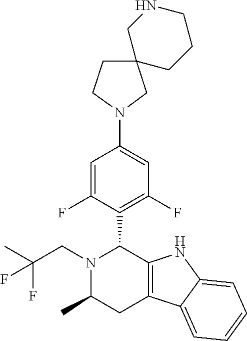 [SMILES: C[C@@H]1Cc2c([nH]c3ccccc23)[C@@H](c2c(F)cc(N3CCC4(CCCNC4)C3)cc2F)N1CC(C)(F)F] | 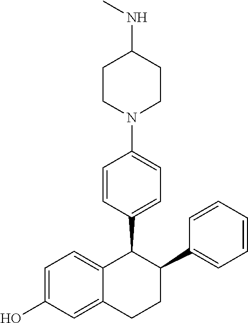 [SMILES: CNC1CCN(c2ccc([C@@H]3c4ccc(O)cc4CC[C@@H]3c3ccccc3)cc2)CC1] |
| 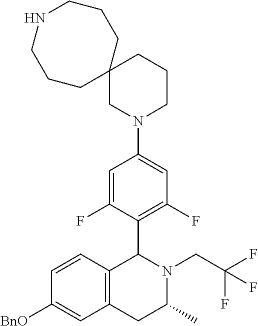 [SMILES: C[C@@H]1Cc2cc(OCc3ccccc3)ccc2C(c2c(F)cc(N3CCCC4(CCCNCCC4)C3)cc2F)N1CC(F)(F)F] | 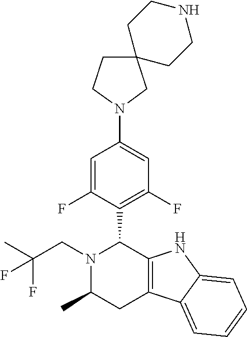 [SMILES: C[C@@H]1Cc2c([nH]c3ccccc23)[C@@H](c2c(F)cc(N3CCC4(CCNCC4)C3)cc2F)N1CC(C)(F)F] | 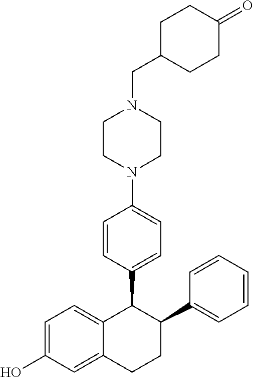 [SMILES: O=C1CCC(CN2CCN(c3ccc([C@@H]4c5ccc(O)cc5CC[C@@H]4c4ccccc4)cc3)CC2)CC1] |

### Methods of Synthesis

In some aspects, the present disclosure provides methods of preparing a Compound of Disclosure.

In some aspects, the present disclosure provides compounds obtainable by, or obtained by, or directly obtained by a method for preparing a Compound of Disclosure.

In some aspects, the present disclosure provides methods of making Compounds of the Disclosure and Intermediates of the Disclosure. Exemplary non-limiting methods of making Compounds of the Disclosure and Intermediates of the Disclosure are provided in General Synthetic Schemes 1-48. See below.

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay involves evaluation of ER degradation activity, e.g., in the human breast cancer cell line T47D and/or MCF7.

In some embodiments, the cell line is maintained and cultured, e.g., in Dulbecco's Modified Eagle's medium (DMEM) (e.g., containing 10% fetal bovine serum, 1 unit/ml of penicillin and 1 μg/ml of streptomycin).

In some embodiments, the luminescence of the cell line is recorded and evaluated.

### Pharmaceutical Compositions

In some aspects, the present disclosure provides pharmaceutical compositions comprising a Compound of Disclosure, or a pharmaceutically acceptable salt, solvate, or stereoisomer thereof, and a pharmaceutically acceptable excipient.

In some embodiments, the pharmaceutically suitable or acceptable carrier is selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. By way of example only, the dose of the compound described herein for methods of treating a disease as described herein is about 0.001 to about 1 mg/kg body weight of the subject per day.

### Kits

In some embodiments, the present disclosure provides kits which comprise a Compound of the Disclosure (or a composition comprising a Compound of the Disclosure) packaged in a manner that facilitates its use to practice methods of the present disclosure. In some embodiments, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In some embodiments, the compound or composition is packaged in a unit dosage form. The kit further can include a device suitable for administering the composition according to the intended route of administration.

### EXEMPLARY EMBODIMENTS

Embodiment 1. A compound of Formula I or a pharmaceutically acceptable salt or solvate thereof, wherein:
A is selected from A-1, A-2, A-3, A-4, and A-5;
   M¹ is selected from 4- to 8-membered heterocyclenyl, phenylenyl, 5-membered heteroarylenyl, and 6-membered heteroarylenyl;
   R^{1a}, R^{1b}, R^{1c}, and R^{1d} are independently selected from hydrogen, halo, hydroxy, and —B(OH)₂;
   R^{2a} is selected from optionally substituted phenyl and optionally substituted C₃-C₈ cycloalkyl; and R^{2b} is hydrogen; or
   R^{2a} and R^{2b} taken together with the carbon atom to which they are attached form a C₃-C₈ cycloalkyl;
   E¹ is selected from —C≡C—, —O—, N(R^{3e})—, and —(CH₂)_{b}—;
   R^{3e} is selected from hydrogen and C₁-C₄ alkyl;
   b is 0, 1, 2, 3, 4, or 5;
   M² is selected from 4- to 8-membered heterocyclenyl, phenylenyl, 5-membered heteroarylenyl, and 6-membered heteroarylenyl;
   R^{4a}, R^{4b}, R^{4c}, and R^{4d} are independently selected from hydrogen, halo, hydroxy, and —B(OH)₂; or
   R^{4a} and R^{4b} taken together with the carbon atoms to which they are attached form an optionally substituted 5- or 6-membered heteroaryl; and R^{4c} and R^{4d} are hydrogen;
   R⁵ is C₁-C₃ alkyl;
   R⁶ is C₁-C₄ haloalkyl;
   E² is selected from —C≡C—, —O—, —N(R^{7e})—, and —(CH₂)_{c}—;
   R^{7e} is selected from hydrogen and C₁-C₄ alkyl;
   c is 0, 1, 2, 3, 4, or 5;
   M³ is selected from 4- to 8-membered heterocyclenyl, phenylenyl, 5-membered heteroarylenyl, and 6-membered heteroarylenyl;
   R^{8a}, R^{8b}, R^{8c}, and R^{8d} are independently selected from hydrogen, halo, hydroxy, and —B(OH)₂;
   R⁹ is C₁-C₃ alkyl;
   R¹⁰ is C₁-C₄ haloalkyl;
   E³ is selected from —C≡C—, —O—, N(R^{11e})—, and —(CH₂)_{d}—;
   R^{11e} is selected from hydrogen and C₁-C₄ alkyl;
   d is 0, 1, 2, 3, 4, or 5;
   R¹² is selected from C₁-C₄ alkyl and C₁-C₄ haloalkyl;
   R^{13a}, R^{13b}, R^{13c}, and R^{13d} are independently selected from hydrogen, halo, hydroxy, and —B(OH)₂;
   each R¹⁴ is independently selected from hydrogen, halo, and hydroxy;
   e is 0, 1, 2, or 3;
   each R¹⁵ is independently selected from hydrogen, halo, and hydroxy;
   f is 0, 1, 2, or 3;
   R^{16a}, R^{16b}, R^{16c}, and R^{16d} are independently selected from hydrogen, halo, hydroxy, and —B(OH)₂;
   R^{17a}, R^{17b}, R^{17c}, R^{17d}, and R^{17e} are independently selected from hydrogen, halo, and hydroxy;
   each R¹⁸ is independently selected from hydrogen, halo, and hydroxy;
   g is 0, 1, 2, or 3;
X is selected from cycloalkylenyl, heterocyclenyl, phenylenyl, and heteroarylenyl;
J is selected from —(CH₂)ₘ—, —(CH₂)_{z1}N(R¹⁹)—, and —(CH₂)_{z2}O—,
   m is 0, 1, 2, or 3;
z1 is 0, 1, or 2;
   z2 is 0, 1, or 2;
   R¹⁹ is selected from hydrogen and C₁-C₄ alkyl;
Y is selected from cycloalkylenyl, heterocyclenyl, heteroarylenyl, —C(═O)— and —(CR^{20a}R^{20b})ᵣ—;
Z is selected from cycloalkylenyl, heterocyclenyl, heteroarylenyl, —C(═O)— and —(CR^{20c}R^{20d})ₛ—;
   each R^{20a}, R^{20b}, R^{20c}, and R^{20d} is independently selected from hydrogen and C₁-C₃ alkyl;
   r is 0, 1, 2, 3, 4, or 5;
   s is 0, 1, 2, 3, 4, or 5;
with the provisos:
   (i) Z is —(CR^{20c}R^{20d})ₛ— when Y is —C(═O)—; or
   (ii) Y is —(CR^{20a}R^{20b})ᵣ— when Z is —C(═O)—;
n is 0, 1, 2, or 3;
B¹ is selected from hydrogen, hydroxy, B¹-1-A, B¹-2-A, B¹-3-A, B¹-4-A, B¹-5-A, B¹-6-A, and B¹-7-A;
   R^{25a} and R^{25b} are independently selected from hydrogen, amino, halo, C₁-C₃ alkyl, and C₁-C₃ alkoxy;
   R²⁶ is selected from hydrogen, deuterium, fluoro, and C₁-C₃ alkyl;
   R²⁷ is selected from hydrogen and C₁-C₃ alkyl;
   Z¹ and Z² are independently selected from —C(═O)— and —CR^{28a}R^{28b}—;
   with the provisos:
      (iv) one of Z¹ or Z² is —C(═O)—; or
      (v) both of Z¹ and Z² are —C(═O)—;
   R^{28a} and R^{28b} are independently selected from hydrogen and C₁-C₃ alkyl; or
   R^{28a} and R^{28b} taken together with the carbon atom to which they are attached form a C₃-C₆ cycloalkyl;
   X¹ is selected from —O—, —S—, and —N(R²⁹)—;
   R²⁹ is selected from hydrogen and C₁-C₄ alkyl;
   t is 1, 2, or 3;
   u is 1, 2, or 3;
   v is 1, 2, or 3; and
   w is 1, 2, or 3.

Embodiment 2. The compound of Embodiment 1, or a pharmaceutically acceptable salt or solvate thereof, wherein A is A-1.

Embodiment 3. The compound of Embodiment 2, or a pharmaceutically acceptable salt or solvate thereof, wherein: M¹ is selected from M¹-1, M¹-2, M¹-3, M¹-4, M¹-5, and M¹-6;
G¹ is selected from —N═ and —CR^{3a}═;
G² is selected from —N═ and —CR^{3b}═;
G³ is selected from —N═ and —CR^{3c}═;
G⁴ is selected from —N═ and —CR^{3d}═; and
R^{3a}, R^{3b}, R^{3c}, R^{3d} are independently selected from hydrogen and halo,
with the proviso that E¹ is —(CH₂)_{b}— when M¹ is M¹-6.

Embodiment 4. The compound of Embodiment 3, or a pharmaceutically acceptable salt or solvate thereof, wherein M¹ is M¹-1.

Embodiment 5. The compound of Embodiment 1, or a pharmaceutically acceptable salt or solvate thereof, wherein A is A-2.

Embodiment 6. The compound of Embodiment 5, or a pharmaceutically acceptable salt or solvate thereof, wherein:
M² is selected from M²-1, M²-2, M²-3, M²-4, M²-5, and M²-6;
G⁵ is selected from —N═ and —CR^{7a}═;
G⁶ is selected from —N═ and —CR^{7b}═;
G⁷ is selected from —N═ and —CR^{7c}═;
G⁸ is selected from —N═ and —CR^{7d}═; and
R^{7a}, R^{7b}, R^{7c}, and R^{7d} are independently selected from hydrogen and halo,
with the proviso that E² is —(CH₂)_{c}— when M² is M²-6.

Embodiment 7. The compound of Embodiment 6, or a pharmaceutically acceptable salt or solvate thereof, wherein M² is M²-1.

Embodiment 8. The compound of Embodiment 1, or a pharmaceutically acceptable salt or solvate thereof, wherein A is A-3.

Embodiment 9. The compound of Embodiment 8, or a pharmaceutically acceptable salt or solvate thereof, wherein:
M³ is selected from M³-1, M³-2, M³-3, M³-4, M³-5, and M³-6;
G⁹ is selected from —N═ and —CR^{11a}═;
G¹⁰ is selected from —N═ and —CR^{11b}═;
G¹¹ is selected from —N═ and —CR^{11c}═;
G¹² is selected from —N═ and —CR^{11d}═; and
R^{11a}, R^{11b}, R^{11c}, and R^{11d} are independently selected from hydrogen and halo,
with the proviso that E³ is —(CH₂)_{d}— when M² is M³-6.

Embodiment 10. The compound of Embodiment 9, or a pharmaceutically acceptable salt or solvate thereof, wherein M³ is M³-1.

Embodiment 11. The compound of Embodiment 1, or a pharmaceutically acceptable salt or solvate thereof, wherein A is A-4 or A-5.

Embodiment 12. The compound of Embodiment 1 of Formula II or a pharmaceutically acceptable salt or solvate thereof, wherein R^{1c} is selected from hydrogen and halo.

Embodiment 13. The compound of Embodiment 1 of Formula III or a pharmaceutically acceptable salt or solvate thereof, wherein R^{1c} is selected from hydrogen and halo.

Embodiment 14. The compound of Embodiments 12 or 13, or a pharmaceutically acceptable salt or solvate thereof, wherein R^{1b} is hydroxy and R^{1c} is hydrogen or fluoro.

Embodiment 15. The compound of Embodiment 14, or a pharmaceutically acceptable salt or solvate thereof, wherein R^{1c} is hydrogen.

Embodiment 16. The compound of any one of Embodiments 12-15, or a pharmaceutically acceptable salt or solvate thereof, wherein E¹ is —O—.

Embodiment 17. The compound of any one of Embodiments 12-15, or a pharmaceutically acceptable salt or solvate thereof, wherein E¹ is —(CH₂)_{b}— and b is 0.

Embodiment 18. The compound of Embodiment 11 of Formula IV or a pharmaceutically acceptable salt or solvate thereof.

Embodiment 19. The compound of Embodiment 18, or a pharmaceutically acceptable salt or solvate thereof, wherein R⁴ is hydroxy.

Embodiment 20. The compound of Embodiments 18 or 19, or a pharmaceutically acceptable salt or solvate thereof, wherein R⁵ is methyl.

Embodiment 21. The compound of Embodiment 1 of Formula V or a pharmaceutically acceptable salt or solvate thereof.

Embodiment 22. The compound of any one of Embodiments 18-21, or a pharmaceutically acceptable salt or solvate thereof, wherein R⁶ is selected from —CH₂CF₂CH₃, —CH₂CF₂H, and —CH₂CF₃.

Embodiment 23. The compound of any one of Embodiments 18-22, or a pharmaceutically acceptable salt or solvate thereof, wherein G⁵ is —N═.

Embodiment 24. The compound of any one of Embodiments 18-22, or a pharmaceutically acceptable salt or solvate thereof, wherein G⁵ is —CR⁷a.

Embodiment 25. The compound of Embodiment 24, a pharmaceutically acceptable salt or solvate thereof, wherein R^{7a} is selected from hydrogen and fluoro.

Embodiment 26. The compound of any one of Embodiments 18-25, or a pharmaceutically acceptable salt or solvate thereof, wherein G⁶ is —N═.

Embodiment 27. The compound of any one of Embodiments 18-25, or a pharmaceutically acceptable salt or solvate thereof, wherein G⁶ is —CR^{7b}═.

Embodiment 28. The compound of Embodiment 27, a pharmaceutically acceptable salt or solvate thereof, wherein R^{7b} is selected from hydrogen and fluoro.

Embodiment 29. The compound of any one of Embodiments 18-28, or a pharmaceutically acceptable salt or solvate thereof, wherein G⁷ is —N═.

Embodiment 30. The compound of any one of Embodiments 18-28, or a pharmaceutically acceptable salt or solvate thereof, wherein G⁷ is —CR^{7c}═.

Embodiment 31. The compound of Embodiment 30, a pharmaceutically acceptable salt or solvate thereof, wherein R^{7c} is selected from hydrogen and fluoro.

Embodiment 32. The compound of any one of Embodiments 18-31, or a pharmaceutically acceptable salt or solvate thereof, wherein G⁸ is —N═.

Embodiment 33. The compound of any one of Embodiments 18-31, or a pharmaceutically acceptable salt or solvate thereof, wherein G⁸ is —CR^{7d}═.

Embodiment 34. The compound of Embodiment 33, a pharmaceutically acceptable salt or solvate thereof, wherein R^{7d} is selected from hydrogen and fluoro.

Embodiment 35. The compound of any one of Embodiments 18-34, or a pharmaceutically acceptable salt or solvate thereof, wherein E² is —(CH₂)_{c}— and c is 0.

Embodiment 36. The compound of Embodiment 1 of Formula VI or a pharmaceutically acceptable salt or solvate thereof.

Embodiment 37. The compound of Embodiment 36, or a pharmaceutically acceptable salt or solvate thereof, wherein R⁹ is methyl.

Embodiment 38. The compound of Embodiments 36 or 37, or a pharmaceutically acceptable salt or solvate thereof, wherein R¹⁰ is selected from —CH₂CF₂CH₃, —CH₂CF₂H, and —CH₂CF₃.

Embodiment 39. The compound of any one of Embodiments 36-38, or a pharmaceutically acceptable salt or solvate thereof, wherein G⁹ is —N═.

Embodiment 40. The compound of any one of Embodiments 36-38, or a pharmaceutically acceptable salt or solvate thereof, wherein G⁹ is —CR^{11a}═.

Embodiment 41. The compound of Embodiment 40, a pharmaceutically acceptable salt or solvate thereof, wherein R^{11a} is selected from hydrogen and fluoro.

Embodiment 42. The compound of any one of Embodiments 36-41, or a pharmaceutically acceptable salt or solvate thereof, wherein G¹⁰ is —N═.

Embodiment 43. The compound of any one of Embodiments 36-41, or a pharmaceutically acceptable salt or solvate thereof, wherein G¹⁰ is —CR^{11b}.

Embodiment 44. The compound of Embodiment 43, a pharmaceutically acceptable salt or solvate thereof, wherein R^{11b} is selected from hydrogen and fluoro.

Embodiment 45. The compound of any one of Embodiments 36-44, or a pharmaceutically acceptable salt or solvate thereof, wherein G¹¹ is —N═.

Embodiment 46. The compound of any one of Embodiments 36-44, or a pharmaceutically acceptable salt or solvate thereof, wherein G¹¹ is —CR^{11c}═.

Embodiment 47. The compound of Embodiment 46, a pharmaceutically acceptable salt or solvate thereof, wherein R^{11c} is selected from hydrogen and fluoro.

Embodiment 48. The compound of any one of Embodiments 36-47, or a pharmaceutically acceptable salt or solvate thereof, wherein G¹² is —N═.

Embodiment 49. The compound of any one of Embodiments 36-47, or a pharmaceutically acceptable salt or solvate thereof, wherein G¹² is —CR^{11d}═.

Embodiment 50. The compound of Embodiment 49, a pharmaceutically acceptable salt or solvate thereof, wherein R^{11d} is selected from hydrogen and fluoro.

Embodiment 51. The compound of any one of Embodiments 36-50, or a pharmaceutically acceptable salt or solvate thereof, wherein E³ is —(CH₂)_{d}— and d is 0.

Embodiment 52. The compound of Embodiment 1 of Formula VII or a pharmaceutically acceptable salt or solvate thereof.

Embodiment 53. The compound of Embodiment 52, or a pharmaceutically acceptable salt or solvate thereof, wherein R¹² is selected from —CH₂CH₃ and —CH₂CH₂Cl.

Embodiment 54. The compound of Embodiments 52 or 53, or a pharmaceutically acceptable salt or solvate thereof, wherein R^{13c} is hydroxy.

Embodiment 55. The compound of Embodiment 1 of Formula VII or a pharmaceutically acceptable salt or solvate thereof.

Embodiment 56. The compound of Embodiment 55, or a pharmaceutically acceptable salt or solvate thereof, wherein R^{16b} is hydroxy.

Embodiment 57. The compound of Embodiments 55 or 56, or a pharmaceutically acceptable salt or solvate thereof, wherein R^{17c} is fluoro.

Embodiment 58. The compound of any one of Embodiments 1-57, or a pharmaceutically acceptable salt or solvate thereof, wherein X is heterocyclenyl.

Embodiment 59. The compound of Embodiment 58, or a pharmaceutically acceptable salt or solvate thereof, wherein X is optionally substituted 4- to 8-membered heterocyclenyl.

Embodiment 60. The compound of Embodiment 59, or a pharmaceutically acceptable salt or solvate thereof, wherein X is selected from

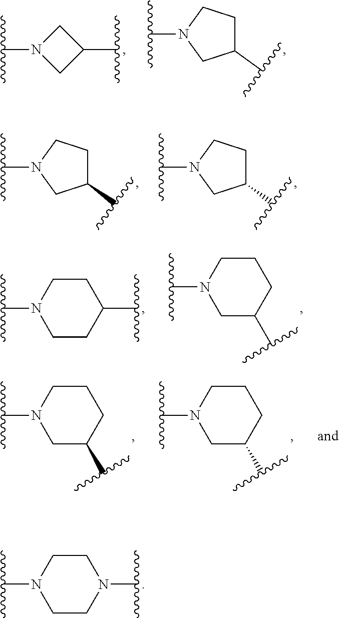

Embodiment 61. The compound of Embodiment 58, or a pharmaceutically acceptable salt or solvate thereof, wherein X is a 7- to 14-membered spiroheterocyclenyl.

Embodiment 62. The compound of Embodiment 58, or a pharmaceutically acceptable salt or solvate thereof, wherein X is

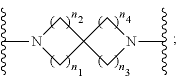

and n₁, n₂, n₃, and n₄ are independently 0, 1, 2, 3, or 4, with the proviso that the sum of n₁, n₂, n₃, and n₄ is 4, 5, 6, 7, 8, 9, or 10.

Embodiment 63. The compound of Embodiment 62, or a pharmaceutically acceptable salt or solvate thereof, wherein X is selected from

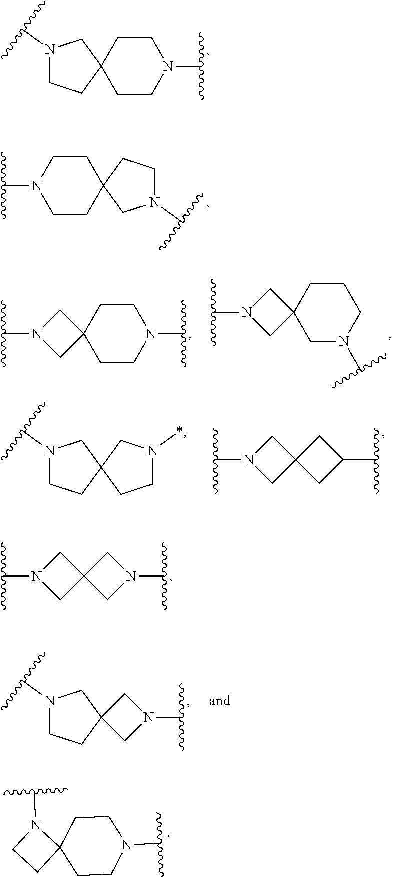

Embodiment 64. The compound of any one of Embodiments 1-63, or a pharmaceutically acceptable salt or solvate thereof, wherein J is —(CH₂)ₘ— and m is 0 or 1.

Embodiment 65. The compound of Embodiment 64, or a pharmaceutically acceptable salt or solvate thereof, wherein m is 0.

Embodiment 66. The compound of any one of Embodiments 1-65, or a pharmaceutically acceptable salt or solvate thereof, wherein Y is selected from heterocyclenyl, —C(═O)— and —(CH₂)ᵣ—.

Embodiment 67. The compound of Embodiment 66, or a pharmaceutically acceptable salt or solvate thereof, wherein Y is optionally substituted 4- to 8-membered heterocyclenyl.

Embodiment 68. The compound of Embodiment 67, or a pharmaceutically acceptable salt or solvate thereof, wherein Y is

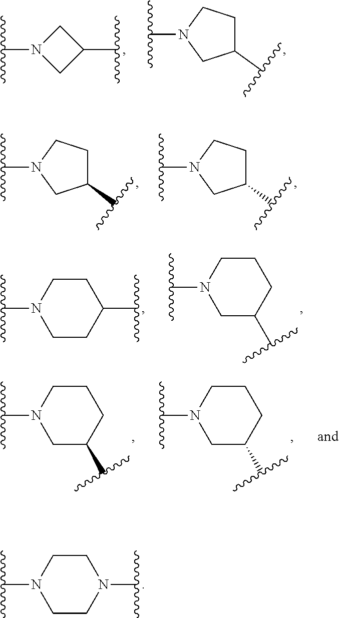

Embodiment 69. The compound of Embodiment 66, or a pharmaceutically acceptable salt or solvate thereof, wherein Y is —(CH₂)ᵣ—.

Embodiment 70. The compound of Embodiment 66, or a pharmaceutically acceptable salt or solvate thereof, wherein Y is —C(═O)—.

Embodiment 71. The compound of any one of Embodiments 1-70, or a pharmaceutically acceptable salt or solvate thereof, wherein Z is selected from heterocyclenyl and —(CH₂)ₛ—.

Embodiment 72. The compound of Embodiment 71, or a pharmaceutically acceptable salt or solvate thereof, wherein Z is optionally substituted 4- to 8-membered heterocyclenyl.

Embodiment 73. The compound of Embodiment 72, or a pharmaceutically acceptable salt or solvate thereof, wherein Z is

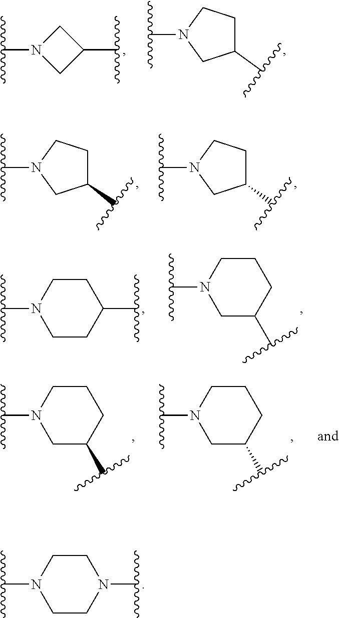

Embodiment 74. The compound of Embodiment 71, or a pharmaceutically acceptable salt or solvate thereof, wherein Z is —(CH₂)ₛ—.

Embodiment 75. The compound of any one of Embodiments 1-69, or a pharmaceutically acceptable salt or solvate thereof, wherein Z is —C(═O)—.

Embodiment 76. The compound of any one of Embodiments 1-75, or a pharmaceutically acceptable salt or solvate thereof, wherein n is 0 or 1.

Embodiment 77. The compound of any one of Embodiments 1-76, or a pharmaceutically acceptable salt or solvate thereof, wherein B¹ is B¹-1-A.

Embodiment 78. The compound of Embodiment 77, or a pharmaceutically acceptable salt or solvate thereof, wherein B¹ is B¹-1-B.

Embodiment 79. The compound of Embodiment 77, or a pharmaceutically acceptable salt or solvate thereof, wherein B¹ is B¹-1-C.

Embodiment 80. The compound of any one of Embodiments 77-79, or a pharmaceutically acceptable salt or solvate thereof, wherein t is 1 or 2.

Embodiment 81. The compound of any one of Embodiments 77-80, or a pharmaceutically acceptable salt or solvate thereof, wherein u is 1.

Embodiment 82. The compound of any one of Embodiments 77-80, or a pharmaceutically acceptable salt or solvate thereof, wherein u is 2.

Embodiment 83. The compound of any one of Embodiments 1-76, or a pharmaceutically acceptable salt or solvate thereof, wherein B¹ is B¹-2-A.

Embodiment 84. The compound of Embodiment 83, or a pharmaceutically acceptable salt or solvate thereof, wherein B¹ is B¹-2-B.

Embodiment 85. The compound of Embodiment 83, or a pharmaceutically acceptable salt or solvate thereof, wherein B¹ is B¹-2-C.

Embodiment 86. The compound of any one of Embodiments 83-85, or a pharmaceutically acceptable salt or solvate thereof, wherein t is 1 or 2.

Embodiment 87. The compound of any one of Embodiments 83-86, or a pharmaceutically acceptable salt or solvate thereof, wherein u is 1.

Embodiment 88. The compound of any one of Embodiments 83-86, or a pharmaceutically acceptable salt or solvate thereof, wherein u is 2.

Embodiment 89. The compound of any one of Embodiments 1-76, or a pharmaceutically acceptable salt or solvate thereof, wherein B¹ is B¹-3-A.

Embodiment 90. The compound of Embodiment 89, or a pharmaceutically acceptable salt or solvate thereof, wherein B¹ is B¹-3-B.

Embodiment 91. The compound of Embodiment 89, or a pharmaceutically acceptable salt or solvate thereof, wherein B¹ is B¹-3-C.

Embodiment 92. The compound of any one of Embodiments 89-91, or a pharmaceutically acceptable salt or solvate thereof, wherein t is 1 or 2.

Embodiment 93. The compound of any one of Embodiments 89-92, or a pharmaceutically acceptable salt or solvate thereof, wherein u is 1.

Embodiment 94. The compound of any one of Embodiments 89-92, or a pharmaceutically acceptable salt or solvate thereof, wherein u is 2.

Embodiment 95. The compound of any one of Embodiments 89-94, or a pharmaceutically acceptable salt or solvate thereof, wherein v is 1.

Embodiment 96. The compound of any one of Embodiments 89-95, or a pharmaceutically acceptable salt or solvate thereof, wherein w is 1.

Embodiment 97. The compound of any one of Embodiments 89-95, or a pharmaceutically acceptable salt or solvate thereof, wherein w is 2.

Embodiment 98. The compound of any one of Embodiments 1-76, or a pharmaceutically acceptable salt or solvate thereof, wherein B¹ is B¹-4-A.

Embodiment 99. The compound of Embodiment 98, or a pharmaceutically acceptable salt or solvate thereof, wherein B¹ is B¹-4-B.

Embodiment 100. The compound of Embodiment 98, or a pharmaceutically acceptable salt or solvate thereof, wherein B¹ is B¹-4-C.

Embodiment 101. The compound of any one of Embodiments 98-100, or a pharmaceutically acceptable salt or solvate thereof, wherein t is 1 or 2.

Embodiment 102. The compound of any one of Embodiments 98-101, or a pharmaceutically acceptable salt or solvate thereof, wherein u is 1.

Embodiment 103. The compound of any one of Embodiments 98-101, or a pharmaceutically acceptable salt or solvate thereof, wherein u is 2.

Embodiment 104. The compound of any one of Embodiments 98-103, or a pharmaceutically acceptable salt or solvate thereof, wherein R²⁷ is hydrogen.

Embodiment 105. The compound of any one of Embodiments 1-76, or a pharmaceutically acceptable salt or solvate thereof, wherein B¹ is B¹-5-A.

Embodiment 106. The compound of Embodiment 105, or a pharmaceutically acceptable salt or solvate thereof, wherein B¹ is B¹-5-B.

Embodiment 107. The compound of Embodiment 105, or a pharmaceutically acceptable salt or solvate thereof, wherein B¹ is B1-s-C.

Embodiment 108. The compound of any one of Embodiments 1-76, or a pharmaceutically acceptable salt or solvate thereof, wherein B1 is B¹-6-A.

Embodiment 109. The compound of Embodiment 108, or a pharmaceutically acceptable salt or solvate thereof, wherein B1 is B¹-6-B.

Embodiment 110. The compound of Embodiment 108, or a pharmaceutically acceptable salt or solvate thereof, wherein B1 is B¹-6-C.

Embodiment 111. The compound of any one of Embodiments 1-76, or a pharmaceutically acceptable salt or solvate thereof, wherein B1 is B¹-7-A.

Embodiment 112. The compound of Embodiment 111, or a pharmaceutically acceptable salt or solvate thereof, wherein B1 is B¹-7-B.

Embodiment 113. The compound of Embodiment 111, or a pharmaceutically acceptable salt or solvate thereof, wherein B1 is B¹-7-C.

Embodiment 114. The compound of any one of Embodiments 105-113, or a pharmaceutically acceptable salt or solvate thereof, wherein X¹ is —O—.

Embodiment 115. The compound of any one of Embodiments 1-114, or a pharmaceutically acceptable salt or solvate thereof, wherein R^{25a} is hydrogen.

Embodiment 116. The compound of any one of Embodiments 1-115, or a pharmaceutically acceptable salt or solvate thereof, wherein R^{25b} is hydrogen.

Embodiment 117. The compound of any one of Embodiments 1-116, or a pharmaceutically acceptable salt or solvate thereof, wherein R²⁶ is hydrogen.

Embodiment 118. The compound of any one of Embodiments 1-117, or a pharmaceutically acceptable salt or solvate thereof, wherein Z¹ is —C(═O)—.

Embodiment 119. The compound of any one of Embodiments 1-117, or a pharmaceutically acceptable salt or solvate thereof, wherein Z¹ is —CH₂—.

Embodiment 120. The compound of any one of Embodiments 1-119, or a pharmaceutically acceptable salt or solvate thereof, wherein Z² is —C(═O)—.

Embodiment 121. The compound of Embodiment 1 that is one or more of the compounds of Table 1, or a pharmaceutically acceptable salt or solvate thereof.

Embodiment 122. A pharmaceutical composition comprising the compound of any one of Embodiments 1-121, or a pharmaceutically acceptable salt or solvate thereof, and a pharmaceutically acceptable carrier.

Embodiment 123. A method of treating cancer in a subject in need thereof, the method comprising administering to the subject a therapeutically effective amount of the compound of any one of Embodiments 1-121, or a pharmaceutically acceptable salt or solvate thereof.

Embodiment 124. The method of Embodiment 123, wherein the cancer is any one or more of the cancers of Table I.

Embodiment 125. The method of Embodiment 124, wherein the cancer is breast cancer.

Embodiment 126. The method of any one of Embodiments 123-125 further comprising administering a therapeutically effective amount of a second therapeutic agent useful in the treatment of the cancer.

Embodiment 127. The pharmaceutical composition of Embodiment 122 for use in treating cancer.

Embodiment 128. The pharmaceutical composition of Embodiment 127, wherein the cancer is any one or more of the cancers of Table I.

Embodiment 129. The pharmaceutical composition of Embodiment 128, wherein the cancer is breast cancer.

Embodiment 130. A compound of any one of Embodiments 1-121, or a pharmaceutically acceptable salt or solvate thereof, for use in treating cancer.

Embodiment 131. The compound for use of Embodiment 130, wherein the cancer is any one or more of the cancers of Table I.

Embodiment 132. The compound for use of Embodiment 131, wherein the cancer is breast cancer.

Embodiment 133. Use of a compound of any one of Embodiments 1-121, or a pharmaceutically acceptable salt or solvate thereof, for the manufacture of a medicament for treatment of cancer.

Embodiment 134. The use of Embodiment 133, wherein the cancer is any one or more of the cancers of Table I.

Embodiment 135. The use of Embodiment 134, wherein the cancer is breast cancer.

Embodiment 136. A method of reducing estrogen receptor protein within a cell of a subject in need thereof, the method comprising administering to the subject a compound of any one of Embodiments 1-121, or a pharmaceutically acceptable salt or solvate thereof.

Embodiment 137. A kit comprising the compound of any one of Embodiments 1-111, or a pharmaceutically acceptable salt or solvate thereof, and instructions for administering the compound, or a pharmaceutically acceptable salt or solvate thereof, to a subject having cancer.

Embodiment 138. The compound of any one of Embodiments 1-76, or a salt or solvate thereof, wherein B¹ is selected from hydrogen or hydroxy.

### Definitions

The term “estrogen receptor protein” or “ER protein” refers to the two main types of estrogen receptor proteins—estrogen receptor alpha (ERα), also known as NR3A1 (nuclear receptor subfamily 3, group A, member 1), and estrogen receptor beta (ERβ) also known as NR3A2 (nuclear receptor subfamily 3, group A, member 2). In humans, ERα and ERβ are encoded by the ESR1 and ESR2 gene, respectively.

The term “a disease or condition wherein degradation of ER proteins provides a benefit” and the like pertains to a disease or condition in which ER proteins and/or an action of ER proteins is important or necessary, e.g., for the onset, progress, expression of that disease or condition, or a disease or a condition which is known to be treated by a ER inhibitor or degrader. Examples of such conditions include, but are not limited to, cancer, a chronic autoimmune disease, an inflammatory disease, a proliferative disease, sepsis, and a viral infection. One of ordinary skill in the art is readily able to determine whether a compound treats a disease or condition mediated by ER proteins for any particular cell type, for example, by assays which conveniently can be used to assess the activity of particular compounds.

The term “second therapeutic agent” refers to a therapeutic agent different from a Compound of the Disclosure and that is known to treat the disease or condition of interest. For example, when a cancer is the disease or condition of interest, the second therapeutic agent can be a known chemotherapeutic drug, like taxol, or radiation, for example. In some embodiments, a Compound of the Disclosure and a second therapeutic agent are administered concurrently (e.g., simultaneously or sequentially). In some embodiments, a Compound of the Disclosure and a second therapeutic agent are administered in temporal proximity.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are degraders of ER proteins and can be used in treating or preventing diseases and conditions wherein degradation of ER provides a benefit.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to subject in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; reduce ER signaling in the target cells; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In some embodiments, the components of the combination therapies are administered at about 1 minute to about 24 hours apart.

As used herein, the term “temporal proximity” refers to that administration of one therapeutic agent (e.g., a Compound of the Disclosure) occurs within a time period before or after the administration of another therapeutic agent (e.g., a second therapeutic agent), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

In the present disclosure, the term “halo” as used by itself or as part of another group refers to —Cl, —F, —Br, or —I.

In the present disclosure, the term “nitro” as used by itself or as part of another group refers to —NO₂.

In the present disclosure, the term “cyano” as used by itself or as part of another group refers to —CN.

In the present disclosure, the term “hydroxy” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In some embodiments, the alkyl is a C₁₋₁₀ alkyl. In some embodiments, the alkyl is a C₁₋₆ alkyl. In some embodiments, the alkyl is a C₁₋₄ alkyl. In some embodiments, the alkyl is a straight chain C₁₋₁₀ alkyl. In some embodiments, the alkyl is a branched chain C₃₋₁₀ alkyl. In some embodiments, the alkyl is a straight chain C₁₋₆ alkyl. In some embodiments, the alkyl is a branched chain C₃₋₆ alkyl. In some embodiments, the alkyl is a straight chain C₁₋₄ alkyl. In some embodiments, the alkyl is a branched chain C₃₋₄ alkyl. In some embodiments, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “optionally substituted alkyl” as used by itself or as part of another group means that the alkyl as defined above is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, carboxy, carboxyalkyl, or cycloalkyl. In some embodiments, the optionally substituted alkyl is either unsubstituted or substituted with one, two, or three substituents, wherein each substituent is nitro, haloalkoxy, aryloxy, aralkyloxy, alkylthio, sulfonamido, alkylcarbonyl, arylcarbonyl, alkylsulfonyl, arylsulfonyl, carboxy, carboxyalkyl, cycloalkyl, or —CHO. In some embodiments, the optionally substituted alkyl is substituted with two substituents. In some embodiments, the optionally substituted alkyl is substituted with one substituent. Non-limiting exemplary optionally substituted alkyl groups include —CH₂CH₂NO₂, —CH₂SO₂CH₃, —CH₂CH₂CO₂H, —CH₂CH₂SO₂CH₃, —CH₂CH₂COPh, —CH₂CH₂CHO, —CH₂CH₂CH₂CHO, and —CH₂CH₂CH₂CH₂CHO.

In the present disclosure, the term “heteroalkyl” as used by itself or part of another group refers to unsubstituted straight- or branched-chain aliphatic hydrocarbons containing from three to twelve chain atoms, i.e., 3- to 12-membered heteroalkyl, or the number of chain atoms designated, wherein at least one —CH₂— is replaced with at least one —O—, —N(H)—, or —S—. The —O—, N(H)—, or —S— can independently be placed at any interior position of the aliphatic hydrocarbon chain so long as each —O—, N(H)—, or —S— group is separated by at least two —CH₂— groups. In some embodiments, one —CH₂— group is replaced with one —O— group. In some embodiments, two —CH₂— groups are replaced with two —O— groups. In some embodiments, three —CH₂— groups are replaced with three —O— groups. In some embodiments, four —CH₂— groups are replaced with four —O— groups. Non-limiting exemplary heteroalkyl groups include —CH₂OCH₃; —CH₂OCH₂CH₂CH₃; —CH₂CH₂CH₂OCH₃; —CH₂OCH₂CH₂OCH₃; and —CH₂OCH₂CH₂OCH₂CH₂OCH₃.

In the present disclosure, the term “cycloalkyl” as used by itself or as part of another group refers to saturated and partially unsaturated (containing one or two double bonds) cyclic aliphatic hydrocarbons containing one, two, or three rings having from three to twelve carbon atoms, i.e., C₃₋₁₂ cycloalkyl, or the number of carbons designated. In some embodiments, the cycloalkyl group has two rings. In some embodiments, the cycloalkyl group has one ring. In some embodiments, the cycloalkyl group is a C₃₋₈ cycloalkyl group. In some embodiments, the cycloalkyl group is a C₃₋₆ cycloalkyl group. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, and cyclohexenyl.

In the present disclosure, the term “optionally substituted cycloalkyl” as used by itself or as part of another group means that the cycloalkyl as defined above is either unsubstituted or substituted with one, two, or three substituents, wherein each substituent is independently halo, nitro, cyano, hydroxy, amino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. In some embodiments, the optionally substituted cycloalkyl is substituted with two substituents. In some embodiments, the optionally substituted cycloalkyl is substituted with one substituent.

In the present disclosure, the term “alkenyl” as used by itself or as part of another group refers to an alkyl group as defined above containing one, two or three carbon-to-carbon double bonds. In some embodiments, the alkenyl group is a C₂₋₆ alkenyl group. In some embodiments, the alkenyl group is a C₂₋₄ alkenyl group. Non-limiting exemplary alkenyl groups include ethenyl, propenyl, isopropenyl, butenyl, sec-butenyl, pentenyl, and hexenyl.

In the present disclosure, the term “optionally substituted alkenyl” as used herein by itself or as part of another group means the alkenyl as defined above is either unsubstituted or substituted with one, two or three substituents, wherein each substituent is independently halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, or heterocyclo.

In the present disclosure, the term “alkynyl” as used by itself or as part of another group refers to an alkyl group as defined above containing one to three carbon-to-carbon triple bonds. In some embodiments, the alkynyl has one carbon-to-carbon triple bond. In some embodiments, the alkynyl group is a C₂₋₆ alkynyl group. In some embodiments, the alkynyl group is a C₂₋₄ alkynyl group. Non-limiting exemplary alkynyl groups include ethynyl, propynyl, butynyl, 2-butynyl, pentynyl, and hexynyl groups.

In the present disclosure, the term “optionally substituted alkynyl” as used herein by itself or as part of another group means the alkynyl as defined above is either unsubstituted or substituted with one, two or three substituents, wherein each substituent is independently halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, or heterocyclo.

In the present disclosure, the term “haloalkyl” as used by itself or as part of another group refers to an alkyl group substituted by one or more fluorine, chlorine, bromine and/or iodine atoms. In some embodiments, the alkyl group is substituted by one, two, or three fluorine and/or chlorine atoms. In some embodiments, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “hydroxyalkyl” as used by itself or as part of another group refers to an alkyl group substituted with one or more, e.g., one, two, or three, hydroxy groups. In some embodiments, the hydroxyalkyl group is a monohydroxyalkyl group, i.e., substituted with one hydroxy group. In some embodiments, the hydroxyalkyl group is a dihydroxyalkyl group, i.e., substituted with two hydroxy groups, e.g.,

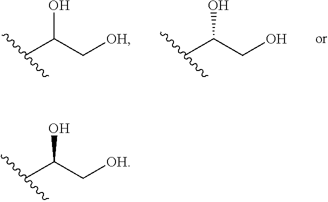 [SMILES: *C(O)CO ; *[C@H](O)CO ; *[C@@H](O)CO]

In some embodiments, the hydroxyalkyl group is a C₁₋₄ hydroxyalkyl group. Non-limiting exemplary hydroxyalkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In some embodiments, the alkoxy group is a C₁₋₄ alkoxy group. In some embodiments, the alkoxy group is a C₁₋₄ alkyl attached to a terminal oxygen atom, e.g., methoxy, ethoxy, tert-butoxy, —OCH₂CH₂C≡CH, and —OCH₂CH₂CH₂C≡CH.

In the present disclosure, the term “alkylthio” as used by itself or as part of another group refers to a sulfur atom substituted by an optionally substituted alkyl group. In some embodiments, the alkylthio group is a C₁₋₄ alkylthio group. Non-limiting exemplary alkylthio groups include —SCH₃, and —SCH₂CH₃.

In the present disclosure, the term “alkoxyalkyl” as used by itself or as part of another group refers to an alkyl group substituted with an alkoxy group. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

In the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic or bicyclic aromatic ring system having from six to fourteen carbon atoms (i.e., C₆-C₁₄ aryl). Non-limiting exemplary aryl groups include phenyl (abbreviated as “Ph”), naphthyl, phenanthryl, anthracyl, indenyl, azulenyl, biphenyl, biphenylenyl, and fluorenyl groups. In some embodiments, the aryl group is phenyl or naphthyl.

In the present disclosure, the term “optionally substituted aryl” as used herein by itself or as part of another group means that the aryl as defined above is either unsubstituted or substituted with one, two, three, four, or five substituents, wherein each substituent is independently halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl.

In some embodiments, the optionally substituted aryl is an optionally substituted phenyl. In some embodiments, the optionally substituted phenyl has four substituents. In some embodiments, the optionally substituted phenyl has three substituents. In some embodiments, the optionally substituted phenyl has two substituents. In some embodiments, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, and 3-chloro-4-fluorophenyl. The term optionally substituted aryl is meant to include bicyclic groups having optionally substituted cycloalkyl or optionally substituted heterocyclo rings fused to a phenyl group. Non-limiting examples include:

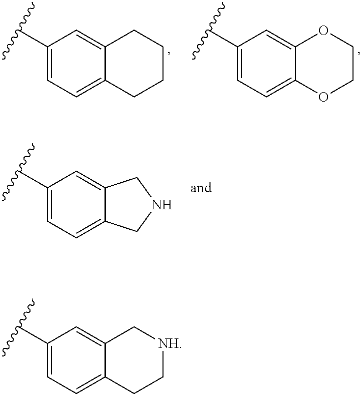 [SMILES: *c1ccc2c(c1)CCCC2 ; *c1ccc2c(c1)OCCO2 ; *c1ccc2c(c1)CNC2 ; *c1ccc2c(c1)CNCC2]

The term “arylenyl” as used herein by itself or part of another group refers to a divalent form of an optionally substituted aryl group. In some embodiments, the arylenyl is a phenylenyl, i.e, a divalent form of an optionally substituted phenyl group. Non-limiting examples include:

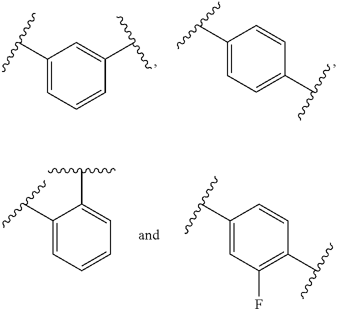 [SMILES: *c1cccc(*)c1 ; *c1ccc(*)cc1 ; *c1ccccc1* ; *c1ccc(*)c(F)c1]

In some embodiments, the arylenyl is an optionally substituted bicyclic 9- to 11-membered arylenyl, i.e., a divalent form of a bicyclic group comprising an optionally substituted pyrrolidine, piperidine, or azepane fused to an optionally substituted phenyl. Non-limiting optionally substituted bicyclic 9- to 11-membered arylenyl groups include:

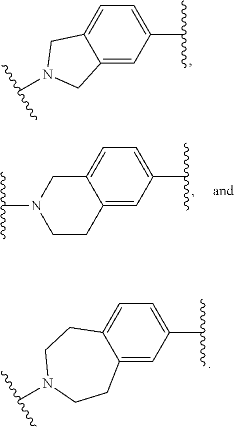 [SMILES: *c1ccc2c(c1)CN(*)C2 ; *c1ccc2c(c1)CCN(*)C2 ; *c1ccc2c(c1)CCN(*)CC2]

In the present disclosure, the term “aryloxy” as used by itself or as part of another group refers to an optionally substituted aryl attached to a terminal oxygen atom. A non-limiting exemplary aryloxy group is PhO—.

In the present disclosure, the term “aralkyloxy” as used by itself or as part of another group refers to an aralkyl group attached to a terminal oxygen atom. A non-limiting exemplary aralkyloxy group is PhCH₂O—.

In the present disclosure, the term “heteroaryl” or “heteroaromatic” refers to monocyclic and bicyclic aromatic ring systems having 5 to 14 ring atoms (i.e., C₅-C₁₄ heteroaryl), wherein at least one carbon atom of one of the rings is replaced with a heteroatom independently selected from oxygen, nitrogen and sulfur. In some embodiments, the heteroaryl contains 1, 2, 3, or 4 heteroatoms independently selected from oxygen, nitrogen and sulfur. In some embodiments, the heteroaryl has three heteroatoms. In some embodiments, the heteroaryl has two heteroatoms. In some embodiments, the heteroaryl has one heteroatom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, P-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In some embodiments, the heteroaryl is thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), or indazolyl (e.g., 1H-indazol-3-yl). The term “heteroaryl” is also meant to include possible N-oxides. A non-limiting exemplary N-oxide is pyridyl N-oxide.

In some embodiments, the heteroaryl is a 5- or 6-membered heteroaryl. In some embodiments, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl.

In some embodiments, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6-membered heteroaryl groups include pyridyl, pyrazinyl, pyrimidinyl, and pyridazinyl.

In the present disclosure, the term “optionally substituted heteroaryl” as used by itself or as part of another group means that the heteroaryl as defined above is either unsubstituted or substituted with one, two, three, or four substituents, e.g., one or two substituents, wherein each substituent is independently halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. In some embodiments, the optionally substituted heteroaryl has one substituent. Any available carbon or nitrogen atom can be substituted. Non-limiting exemplary optionally substituted 5-membered heteroaryl groups include, but are not limited to:

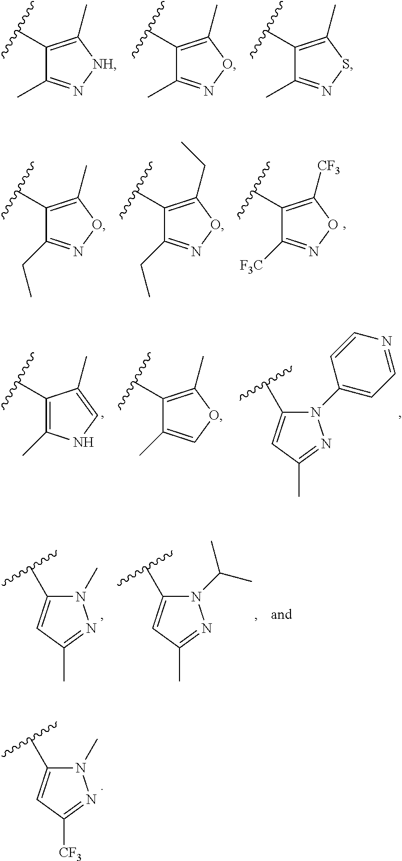

The term optionally substituted heteroaryl is also meant to include bicyclic groups having optionally substituted cycloalkyl or optionally substituted heterocyclo rings fused to a heteroaryl group. Non-limiting examples include:

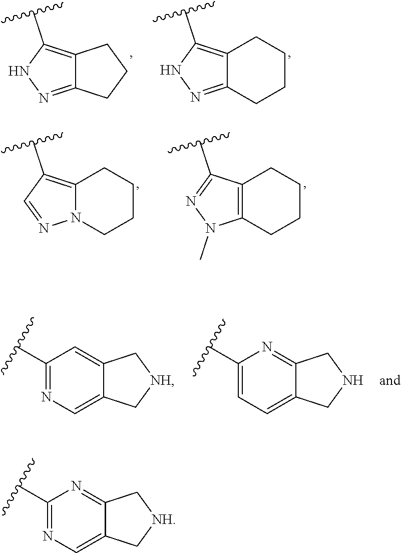

The term “heteroarylenyl” as used herein by itself or part of another group refers to a divalent form of an optionally substituted heteroaryl group. In some embodiments, the heteroarylenyl is an optionally substituted 5-membered heteroarylenyl, i.e., a divalent form of an optionally substituted 5-membered heteroaryl group. In some embodiments, the substituent(s), if present, on any available carbon atom(s) of the 5-membered heteroarylenyl is(are) independently halo, C₁-C₆ alkyl, C₁-C₄ haloalkyl, C₁-C₄ alkoxy, (hydroxy)C₁-C₄ alkyl, or C₃-C₆ cycloalkyl; and/or the substituent, if present, on any available nitrogen atom is C₁-C₆ alkyl. Non-limiting exemplary 5-membered heteroarylenyl groups include:

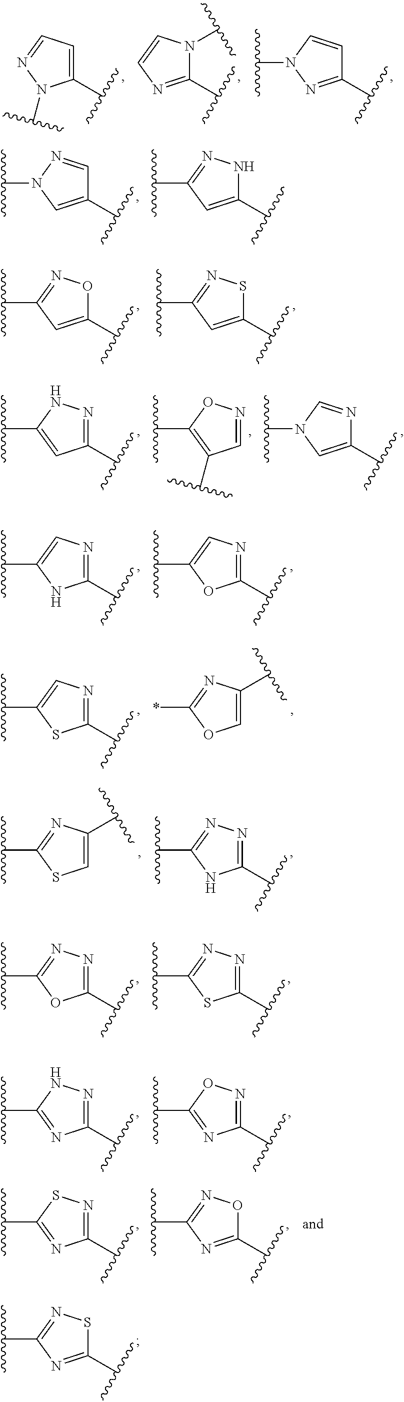

In some embodiments, the heteroarylenyl is an optionally substituted 6-membered heteroarylenyl, i.e., a divalent form of an optionally substituted 6-membered heteroaryl group. In some embodiments, the substituent(s), if present, on any available carbon atom(s) of the 6-membered heteroarylenyl is(are) independently halo, C₁-C₆ alkyl, C₁-C₄ haloalkyl, C₁-C₄ alkoxy, (hydroxy)C₁-C₄ alkyl, or C₃-C₆ cycloalkyl. Non-limiting exemplary 6-membered heteroarylenyl groups include:

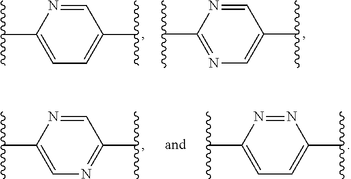

In some embodiments, the heteroarylenyl is an optionally substituted bicyclic 9- to 11-membered heteroarylenyl, i.e., a divalent form of an optionally substituted bicylic 9- to 11-membered heteroaryl group comprising an optionally substituted pyrrolidine, piperidine, or azepane fused to an optionally substituted 5- or 6-membered heteroaryl. In some embodiments, the substituent(s), if present, on any available carbon atom(s) of the bicyclic 9- to 11-membered heteroarylenyl is(are) independently halo, C₁-C₆ alkyl, C₁-C₄ haloalkyl, C₁-C₄ alkoxy, (hydroxy)C₁-C₄ alkyl, or C₃-C₆ cycloalkyl; Non-limiting exemplary optionally substituted bicyclic 9- to 11-membered heteroarylenyl groups include:

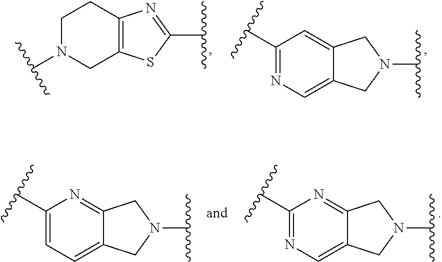

In the present disclosure, the term “heterocycle” or “heterocyclo” as used by itself or as part of another group refers to saturated and partially unsaturated (e.g., containing one or two double bonds) cyclic groups containing one, two, or three rings (e.g., fused, bridged, or spiro) having from three to fourteen ring members (i.e., a 3- to 14-membered heterocyclo) wherein at least one carbon atom of one of the rings is replaced with a heteroatom. Each heteroatom is independently selected from oxygen, sulfur, including sulfoxide and sulfone, and/or nitrogen atoms, which can be oxidized or quaternized. The term “heterocyclo” is meant to include groups wherein a ring —CH₂— is replaced with a —C(═O)—, for example, cyclic ureido groups such as 2-imidazolidinone and cyclic amide groups such as P-lactam, y-lactam, 6-lactam, F-lactam, and piperazin-2-one. The term “heterocyclo” is also meant to include groups having fused optionally substituted aryl groups, e.g., indolinyl, chroman-4-yl. In some embodiments, the heterocyclo group is a 4-, 5- or 6-membered cyclic group containing one ring and one or two oxygen and/or nitrogen atoms. The heterocyclo can be optionally linked to the rest of the molecule through any available carbon or nitrogen atom. Non-limiting exemplary heterocyclo groups include dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl.

In the present disclosure, the term “optionally substituted heterocyclo” as used herein by itself or part of another group means the heterocyclo as defined above is either unsubstituted or substituted with one, two, three, or four substituents wherein each substituent is independently halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, alkoxycarbonyl, CF₃C(═O)—, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. Substitution may occur on any available carbon or nitrogen atom, or both. Non-limiting exemplary optionally substituted heterocyclo groups include:

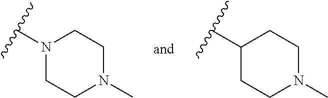

In some embodiments, the heterocyclo group is a spiroheterocyclo. The term “spiroheterocyclo” as used herein by itself or part of another group refers to an optionally substituted heterocyclo group containing seven to fourteen ring members, wherein:
(i) a first and second ring are connected through a quaternary carbon atom, i.e., a spirocarbon;
(ii) the first ring is an optionally substituted mono- or bicyclic heterocyclo containing a nitrogen atom; and
(iii) the second ring is either:
   (a) an optionally substituted mono- or bicyclic cycloalkyl; or
   (b) an optionally substituted mono- or bicyclic heterocyclo containing a nitrogen atom.

In some embodiments, the first ring is an optionally substituted monocyclic 4- to 9-membered heterocyclo containing a nitrogen atom. In some embodiments, the second ring is an optionally substituted monocyclic C₃₋₈ cycloalkyl. In some embodiments, the second ring is an optionally substituted monocyclic 4- to 9-membered heterocyclo containing a nitrogen atom. Non-limiting exemplary spiroheterocyclo groups include:

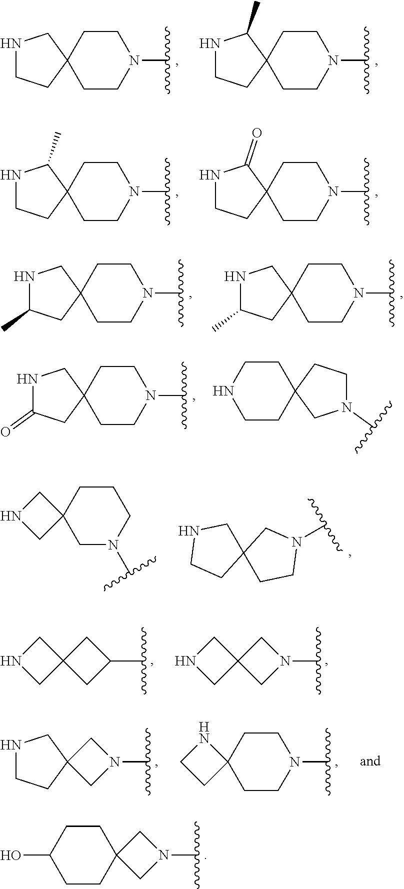

The term “heterocyclenyl” as used herein by itself or part of another group refers to a divalent form of an optionally substituted heterocyclo group. In some embodiments, the heterocyclenyl is an optionally substituted 4- to 8-membered heterocyclenyl, i.e., a divalent form of an optionally substituted monocyclic or bicyclic 4- to 8-membered heterocyclo. In some embodiments, the heterocyclenyl is an optionally substituted 4- to 6-membered heterocyclenyl, i.e., a divalent form of an optionally substituted monocyclic 4- to 6-membered heterocyclo. In some embodiments, the heterocyclenyl is an optionally substituted 7- to 14-membered spiroheterocyclenyl, i.e., a is a divalent form of an optionally substituted 7- to 14-membered spiroheterocyclo. In some embodiments, the heterocyclenyl is a divalent form of an optionally substituted azetidine. In some embodiments, the heterocyclenyl is a divalent form of an optionally substituted pyrrolidine. In some embodiments, the heterocyclenyl is a divalent form of an optionally substituted piperidinyl. In some embodiments, the heterocyclenyl is a divalent form of an optionally substituted bicyclic 8-membered heterocyclenyl. Non-limiting exemplary 4- to 8-membered heterocyclenyl groups include:

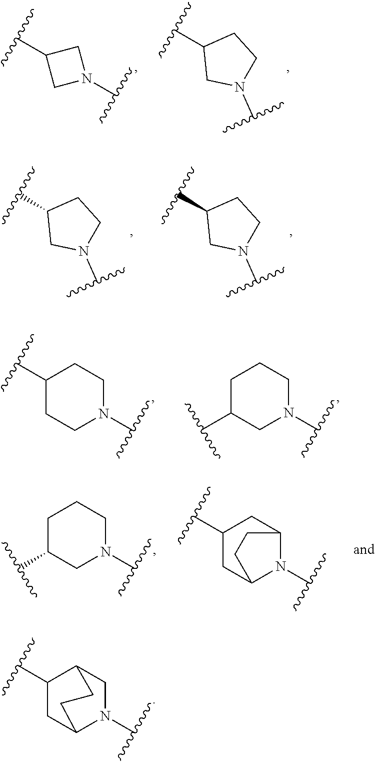

Non-limiting exemplary 7- to 14-membered spiroheterocyclenyl groups include:

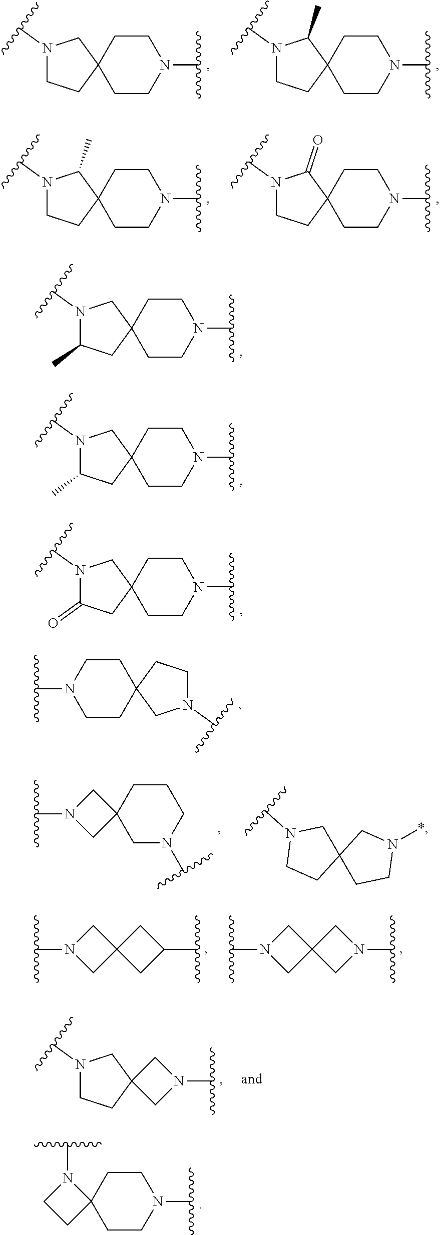

The term “alkylenyl” as used herein by itself or part of another group refers to a divalent form of an alkyl group. In some embodiments, the alkylenyl is a divalent form of a C₁₋₁₂ alkyl, i.e., a C₁-C₁₂ alkylenyl. In some embodiments, the alkylenyl is a divalent form of a C₁₋₁₀ alkyl, i.e., a C₁-C₁₀ alkylenyl. In some embodiments, the alkylenyl is a divalent form of a C₁₋₈ alkyl, i.e., a C₁-C₈ alkylenyl. In some embodiments, the alkylenyl is a divalent form of an unsubstituted C₁₋₆ alkyl, i.e., a C₁-C₆ alkylenyl. In some embodiments, the alkylenyl is a divalent form of an unsubstituted C₁₋₄ alkyl, i.e., a C₁-C₄ alkylenyl. Non-limiting exemplary alkylenyl groups include —CH₂—, —CH₂CH₂—, —CH₂CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In some embodiments, the heteroalkylenyl is a divalent form of a 3- to 20-membered heteroalkyl, i.e., a 3- to 20-membered heteroalkylenyl. In some embodiments, the heteroalkylenyl is a divalent form of a 3- to 10-membered heteroalkyl, i.e., a 3- to 10-membered heteroalkylenyl. In some embodiments, the heteroalkylenyl is a divalent form of a 3- to 8-membered heteroalkyl, i.e., a 3- to 8-membered heteroalkylenyl. In some embodiments, the heteroalkylenyl is a divalent form of a 3- to 6-membered heteroalkyl, i.e., a 3- to 6-membered heteroalkylenyl. In some embodiments, the heteroalkylenyl is a divalent form of a 3- or 4-membered heteroalkyl, i.e., a 3- or 4-membered heteroalkylenyl. In some embodiments, the heteroalkylenyl is a radical of the formula —(CH₂CH₂O)ᵤ₁— wherein u₁ is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “cycloalkylenyl” as used herein by itself or part of another group refers to a divalent form of an optionally substituted C₃-C₁₂ cycloalkyl group. In some embodiments, the cycloalkylenyl is a C₄-C₈ cycloalkylenyl, i.e., a divalent form of an optionally substituted monocyclic or bicyclic C₄-C₈ cycloalkyl. In some embodiments, the cycloalkylenyl is a monocyclic or bicyclic 4- to 6-membered cycloalkylenyl. In some embodiments, the cycloalkylenyl is a monocyclic or bicyclic 5-membered cycloalkylenyl. In some embodiments, the cycloalkylenyl is a 6-membered cycloalkylenyl. Non-limiting exemplary groups include:

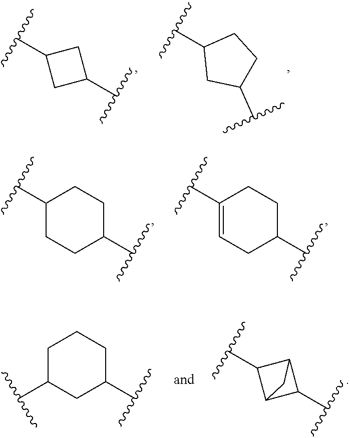 [SMILES: *C1CC(*)C1 ; *C1CCC(*)C1 ; *C1CCC(*)CC1 ; *C1=CCC(*)CC1 ; *C1CCCC(*)C1 ; *C1C2CC1C2*]

In the present disclosure, the term “amino” as used by itself or as part of another group refers to —NR^{100a}R^{100b}, wherein R^{100a} and R^{100b} are each independently hydrogen, optionally substituted alkyl, alkynyl, haloalkyl, hydroxyalkyl, optionally substituted cycloalkyl, optionally substituted aryl, optionally substituted heterocyclo, or optionally substituted heteroaryl, or R^{100a} and R^{100b} are taken together to form a 3- to 8-membered optionally substituted heterocyclo. Non-limiting exemplary amino groups include —NH₂ and —N(H)(CH₃).

In the present disclosure, the term “(amino)alkyl” as used by itself or as part of another group refers to an alkyl group substituted with an amino group. Non-limiting exemplary amino alkyl groups include —CH₂CH₂NH₂, and —CH₂CH₂N(H)CH₃, —CH₂CH₂N(CH₃)₂, and —CH₂N(H)cyclopropyl.

In the present disclosure, the term “carboxamido” as used by itself or as part of another group refers to a radical of formula —C(═O)NR^{101a}R^{101b}, wherein R^{101a} and R^{101b} are each independently hydrogen, optionally substituted alkyl, hydroxyalkyl, optionally substituted cycloalkyl, optionally substituted aryl, optionally substituted heterocyclo, or optionally substituted heteroaryl, or R^{101a} and R^{101b} taken together with the nitrogen to which they are attached form a 3- to 8-membered optionally substituted heterocyclo group. In some embodiments, R^{101a} and R^{101b} are each independently hydrogen or optionally substituted alkyl. In some embodiments, R^{101a} and R^{101b} are taken together to taken together with the nitrogen to which they are attached form a 3- to 8-membered optionally substituted heterocyclo group. Non-limiting exemplary carboxamido groups include, but are not limited to, —CONH₂, —CON(H)CH₃, —CON(CH₃)₂, —CON(H)Ph,

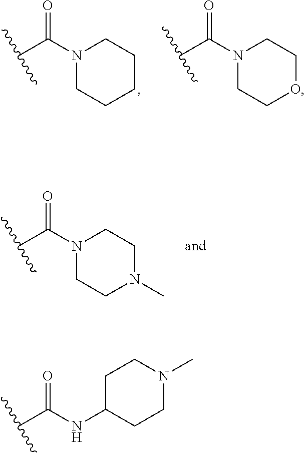

In the present disclosure, the term “sulfonamido” as used by itself or as part of another group refers to a radical of the formula —SO₂NR^{102a}R^{102b}, wherein R^{102a} and R^{102b} are each independently hydrogen, optionally substituted alkyl, or optionally substituted aryl, or R^{102a} and R^{102b} taken together with the nitrogen to which they are attached from a 3- to 8-membered heterocyclo group. Non-limiting exemplary sulfonamido groups include —SO₂NH₂, —SO₂N(H)CH₃, and —SO₂N(H)Ph.

In the present disclosure, the term “alkylcarbonyl” as used by itself or as part of another group refers to a carbonyl group, i.e., —C(═O)—, substituted by an alkyl group. A non-limiting exemplary alkylcarbonyl group is —COCH₃.

In the present disclosure, the term “arylcarbonyl” as used by itself or as part of another group refers to a carbonyl group, i.e., —C(═O)—, substituted by an optionally substituted aryl group. A non-limiting exemplary arylcarbonyl group is —COPh.

In the present disclosure, the term “alkoxycarbonyl” as used by itself or as part of another group refers to a carbonyl group, i.e., —C(═O)—, substituted by an alkoxy group.

Non-limiting exemplary alkoxycarbonyl groups include —C(═O)OMe, —C(═O)OEt, and —C(═O)OtBu.

In the present disclosure, the term “alkylsulfonyl” as used by itself or as part of another group refers to a sulfonyl group, i.e., —SO₂—, substituted by any of the above-mentioned optionally substituted alkyl groups. A non-limiting exemplary alkylsulfonyl group is —SO₂CH₃.

In the present disclosure, the term “arylsulfonyl” as used by itself or as part of another group refers to a sulfonyl group, i.e., —SO₂—, substituted by any of the above-mentioned optionally substituted aryl groups. A non-limiting exemplary arylsulfonyl group is —SO₂Ph.

In the present disclosure, the term “mercaptoalkyl” as used by itself or as part of another group refers to any of the above-mentioned alkyl groups substituted by a —SH group.

In the present disclosure, the term “carboxy” as used by itself or as part of another group refers to a radical of the formula —COOH.

In the present disclosure, the term “carboxyalkyl” as used by itself or as part of another group refers to any of the above-mentioned alkyl groups substituted with a —COOH. A non-limiting exemplary carboxyalkyl group is —CH₂CO₂H.

In the present disclosure, the terms “aralkyl” or “arylalkyl” as used by themselves or as part of another group refers to an alkyl group substituted with one, two, or three optionally substituted aryl groups. In some embodiments, the optionally substituted aralkyl group is a C₁₋₄ alkyl substituted with one optionally substituted aryl group. In some embodiments, the optionally substituted aralkyl group is a C₁₋₃ alkyl substituted with one optionally substituted phenyl group, i.e., an “(optionally substituted phenyl)C₁-C₃ alkyl.” In some embodiments, the optionally substituted aralkyl group is a C₁ or C₂ alkyl substituted with one optionally substituted aryl group. In some embodiments, the optionally substituted aralkyl group is a C₁ or C₂ alkyl substituted with one optionally substituted phenyl group. Non-limiting exemplary optionally substituted aralkyl groups include benzyl, phenethyl, —CHPh₂, —CH₂(4-F-Ph), —CH—₂(4-Me-Ph), —CH₂(4-CF₃-Ph), and —CH(4-F-Ph)₂.

In the present disclosure, the term “(heteroaryl)alkyl” as used by itself or part of another group refers to an alkyl group substituted with an optionally substituted heteroaryl group. In some embodiments, the (heteroaryl)alkyl is a C₁₋₃ alkyl substituted with one optionally substituted 5-membered heteroaryl group, i.e., an “(optionally substituted 5-membered heteroaryl)C₁-C₃ alkyl.” In some embodiments, the (heteroaryl)alkyl is a C₁₋₃ alkyl substituted with one optionally substituted 6-membered heteroaryl group, i.e., an “(optionally substituted 6-membered heteroaryl)C₁-C₃ alkyl.” Non-limiting exemplary (heteroaryl)alkyl groups include:

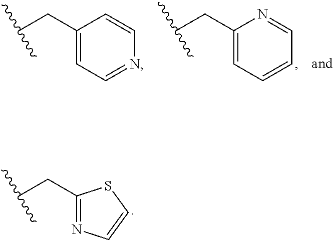

In the present disclosure, the term “(heterocyclo)alkyl” as used by itself or part of another group refers to an alkyl group substituted with an optionally substituted heterocyclo group. In some embodiments, the (heterocyclo)alkyl is a C₁₋₄ alkyl substituted with one optionally substituted heterocyclo group. Non-limiting exemplary (heterocyclo)alkyl groups include:

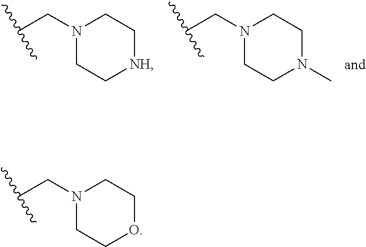

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

Representative Compounds of the Disclosure were prepared according to the following General Synthetic Schemes.

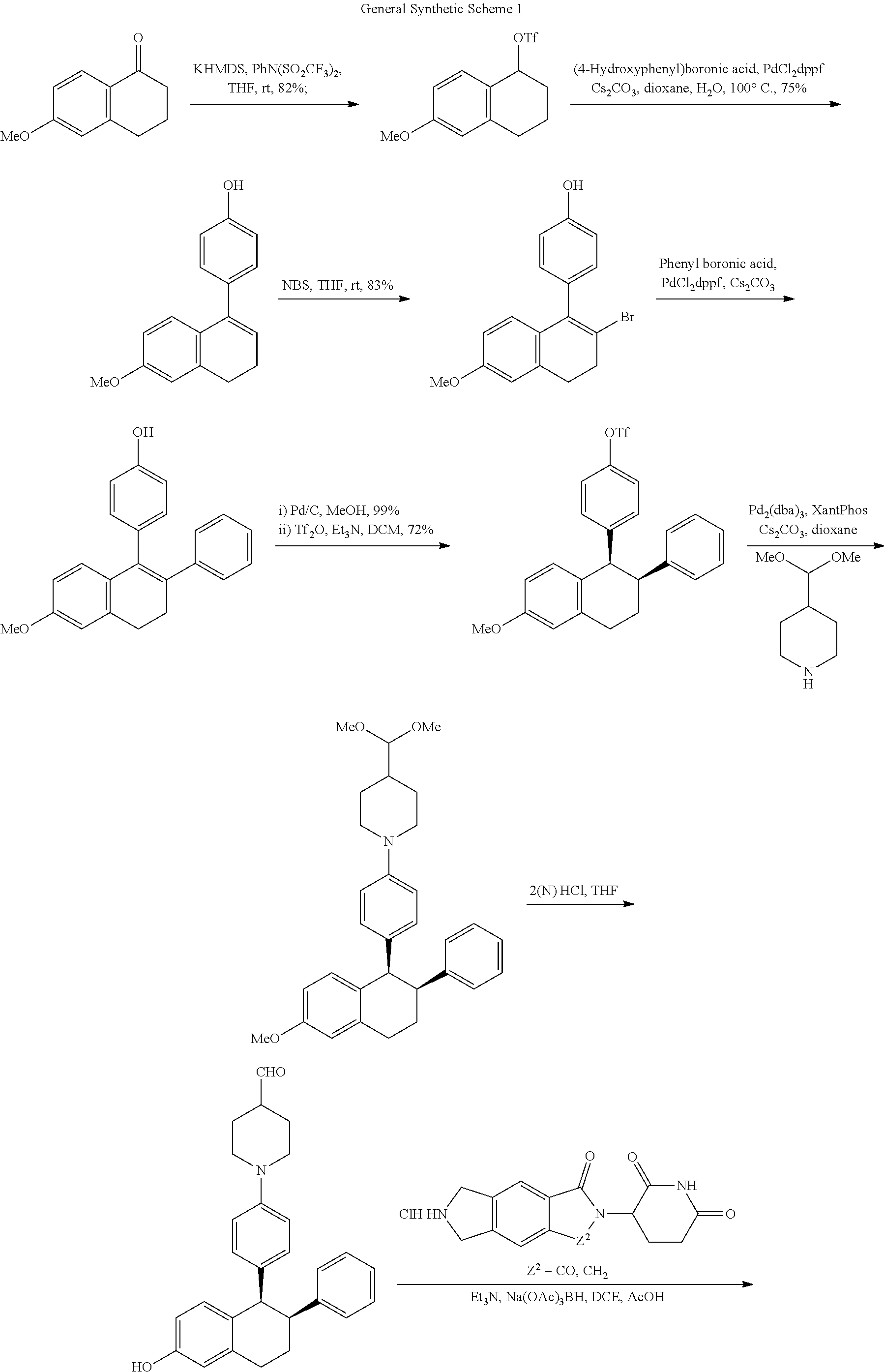

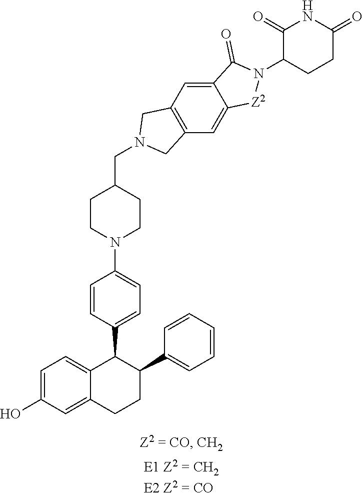

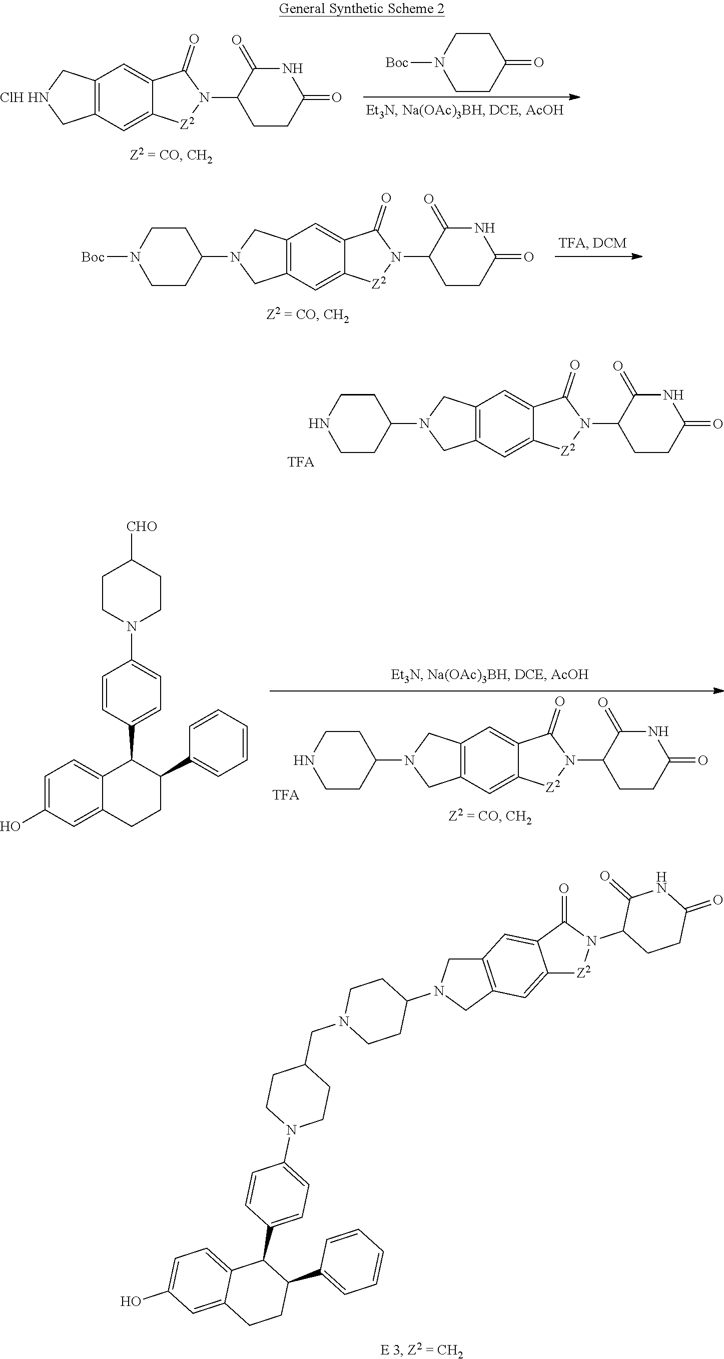

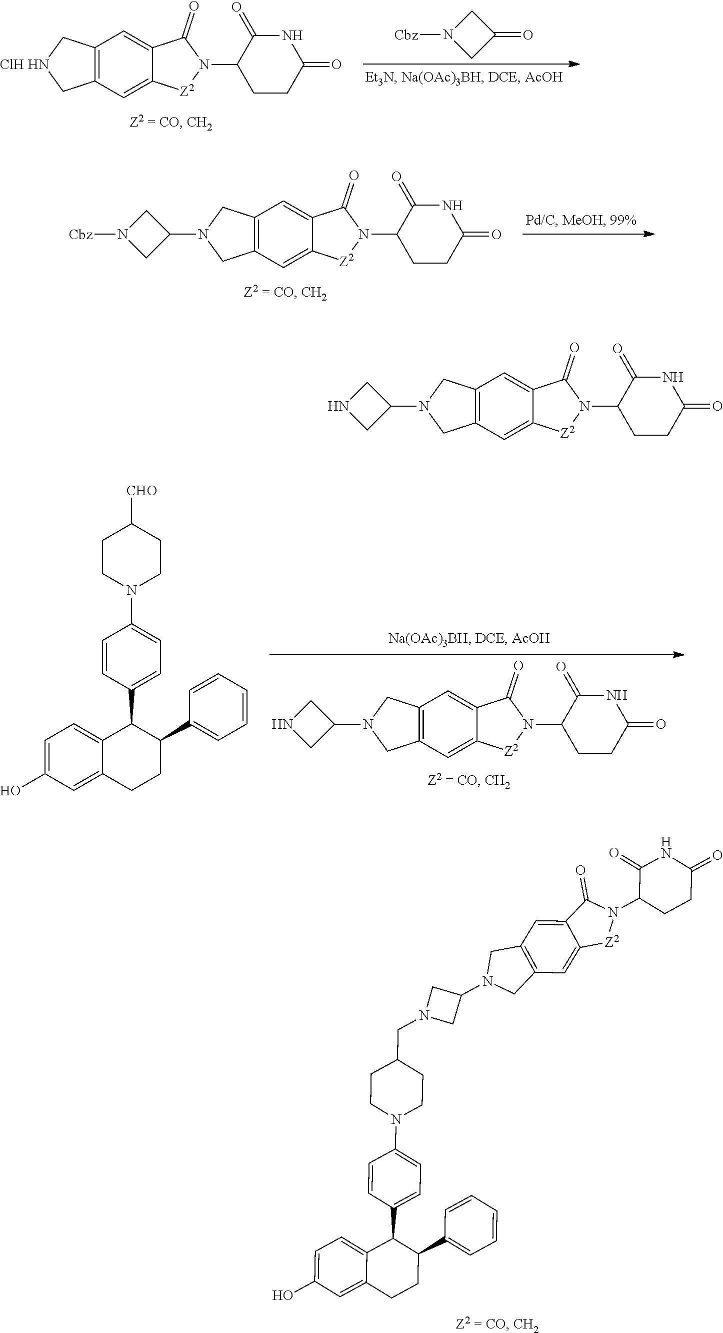

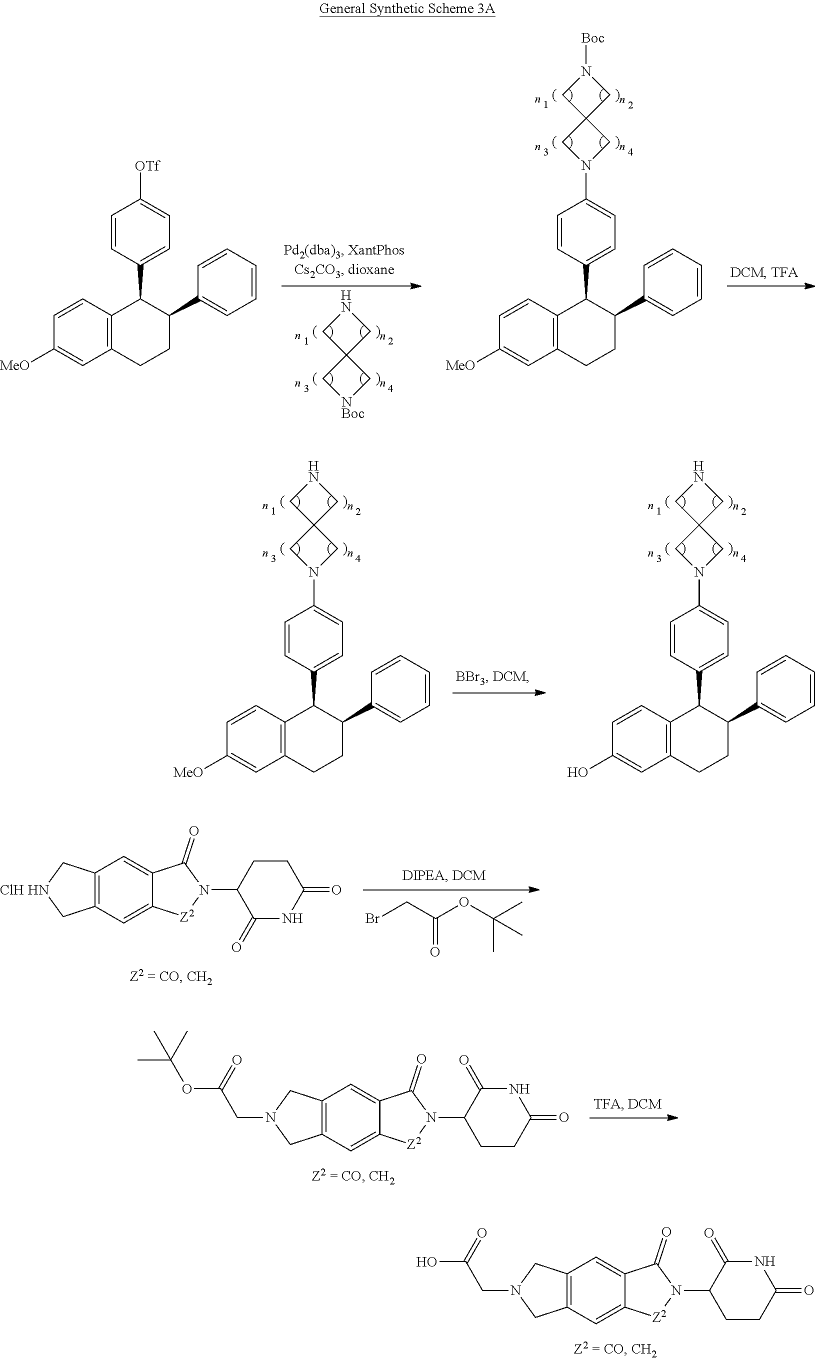

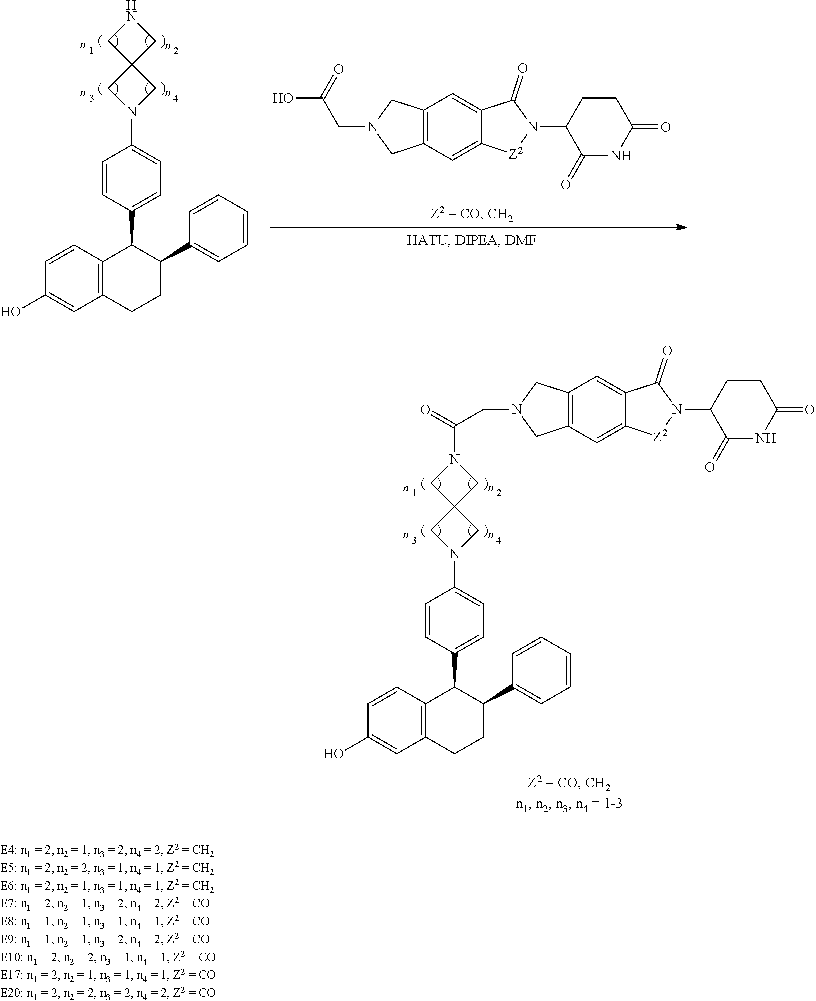

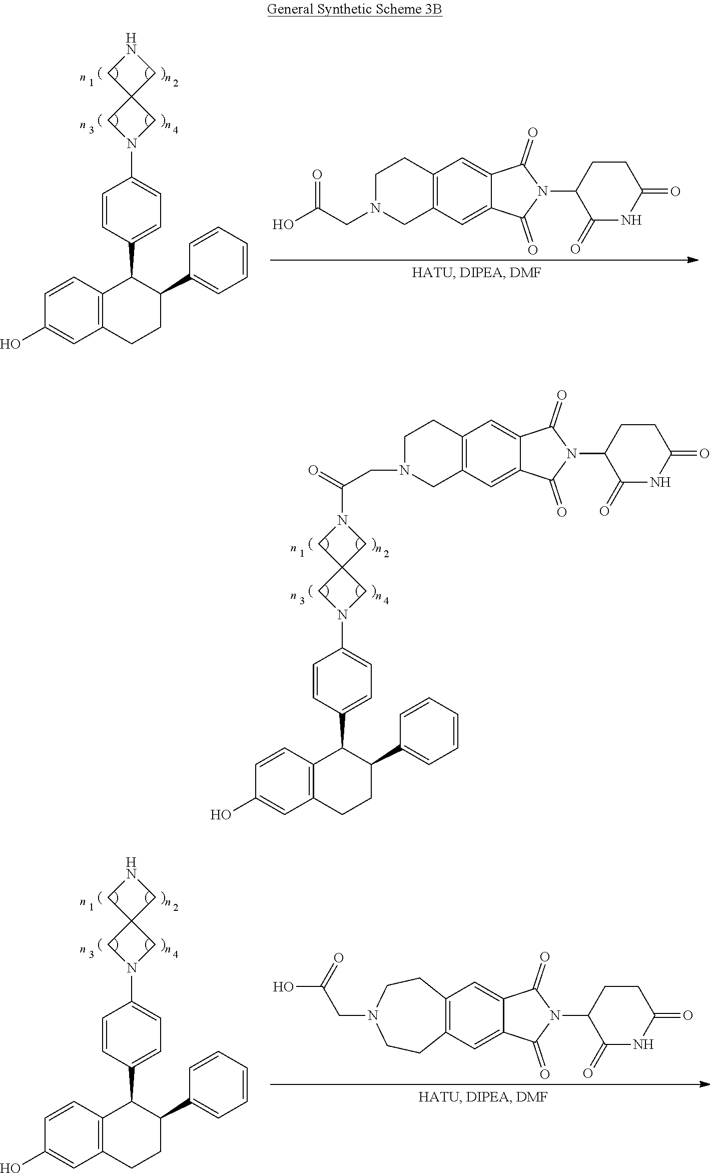

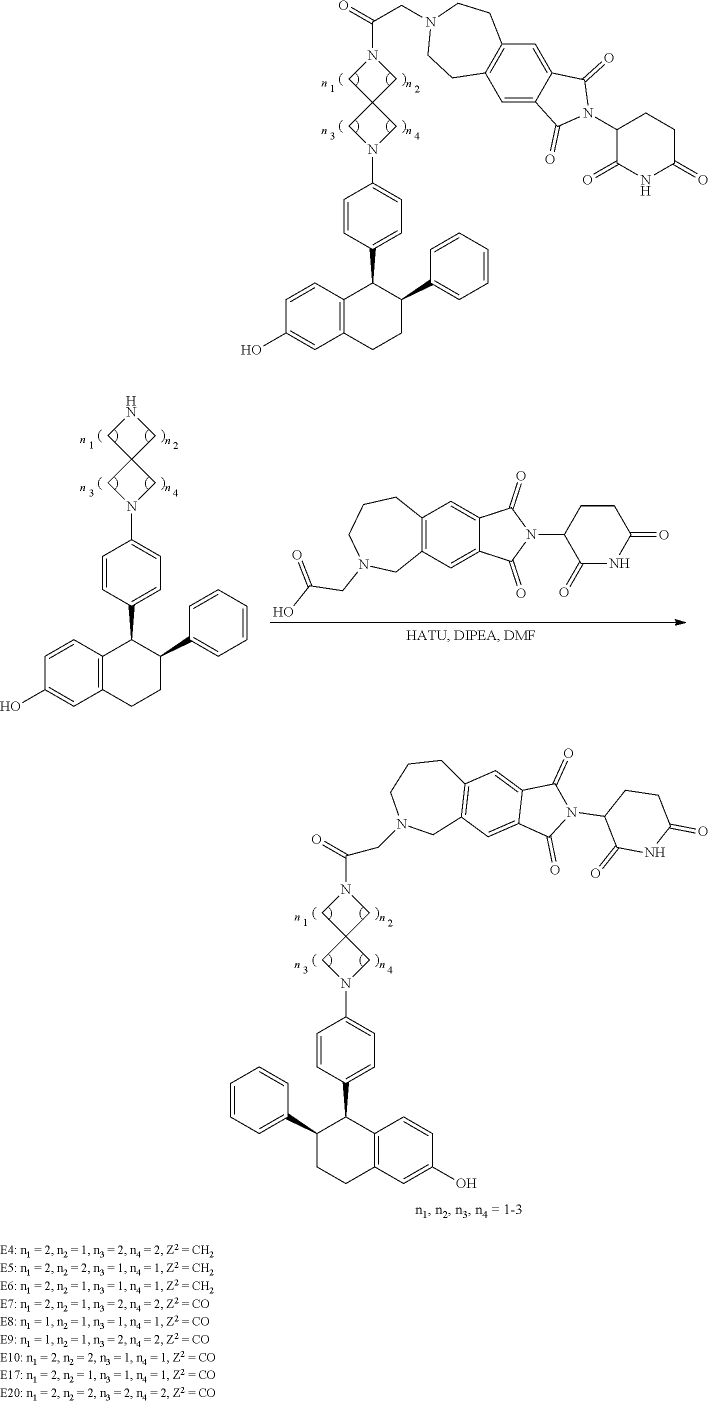

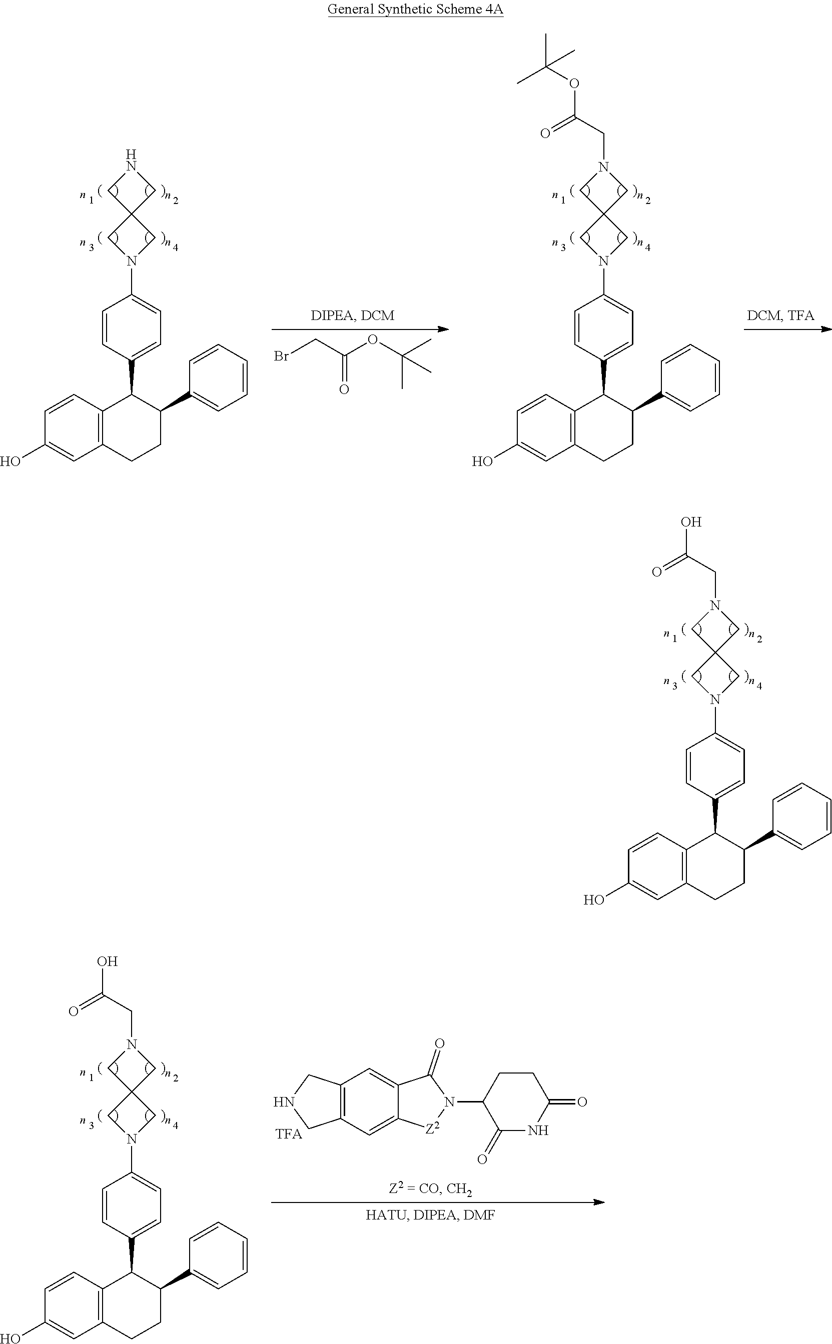

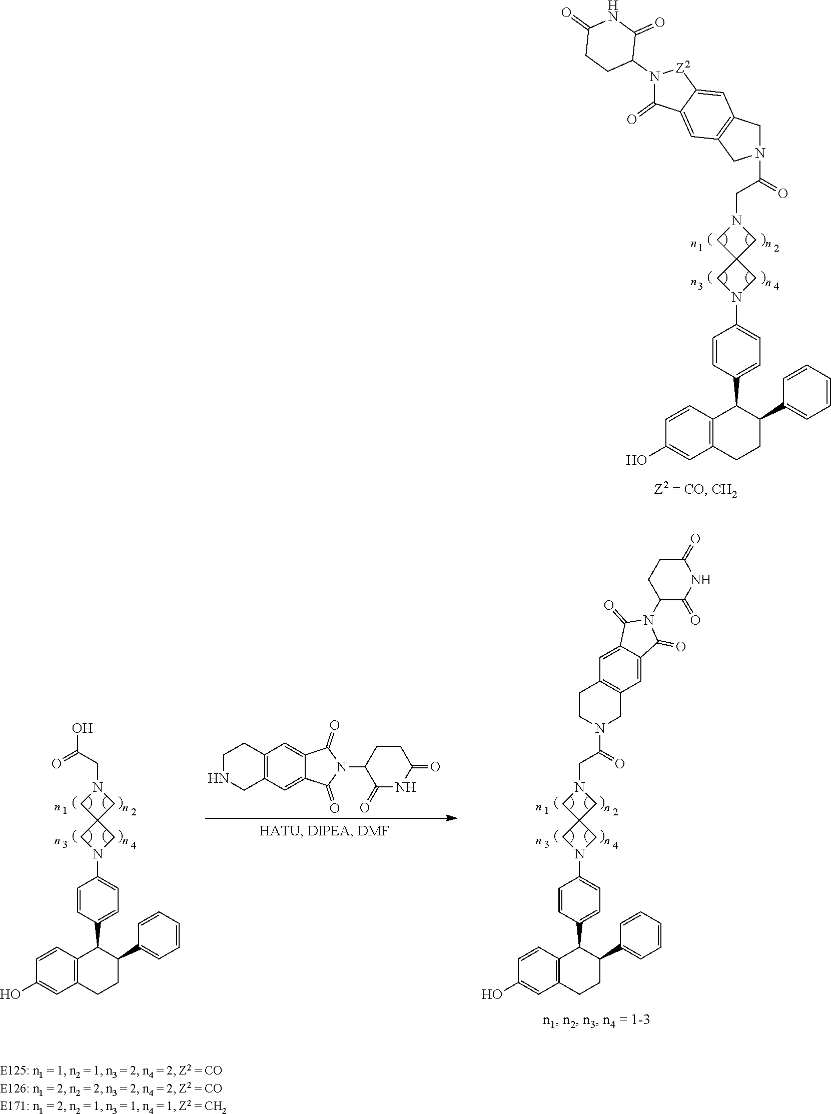

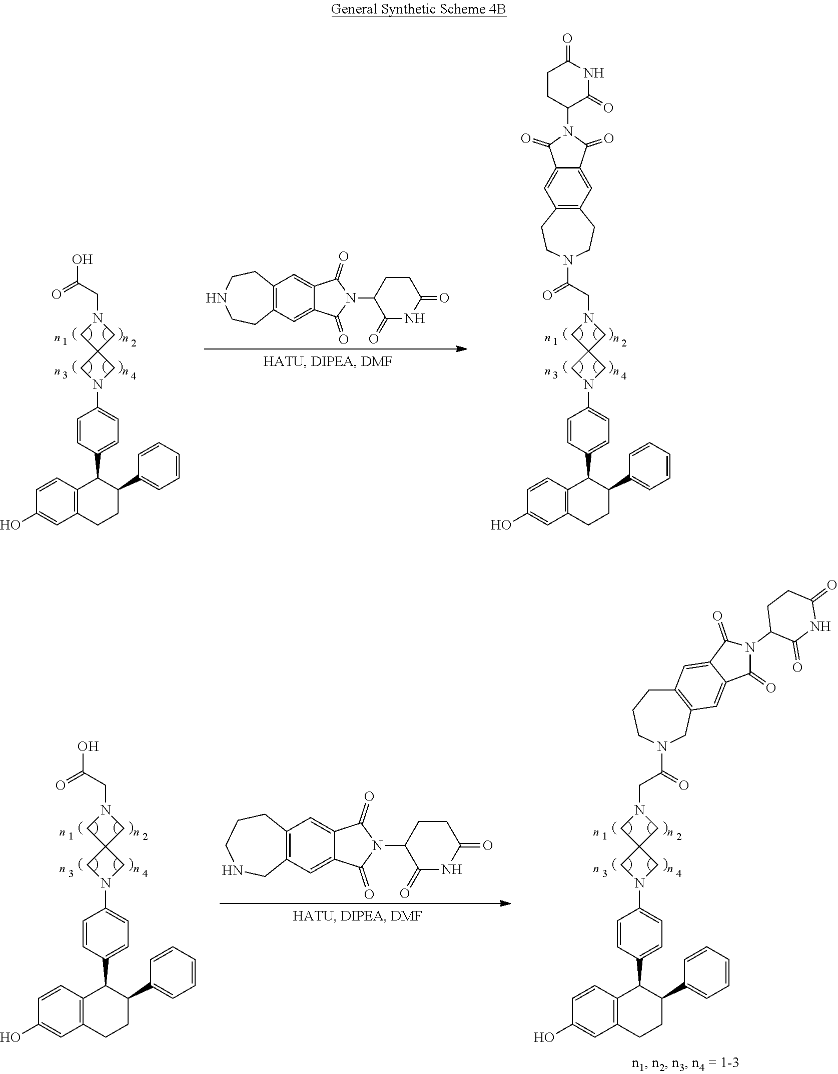

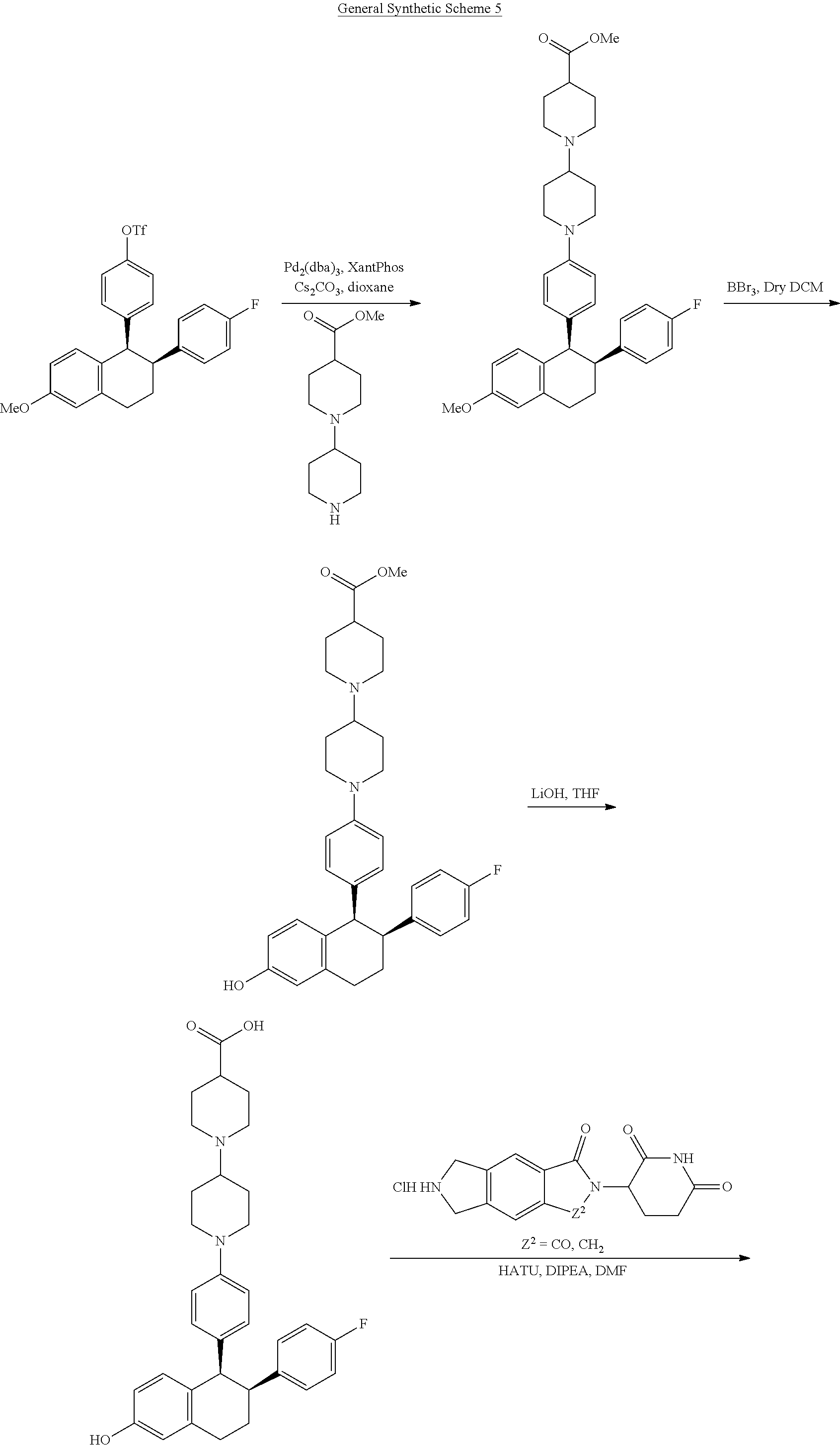

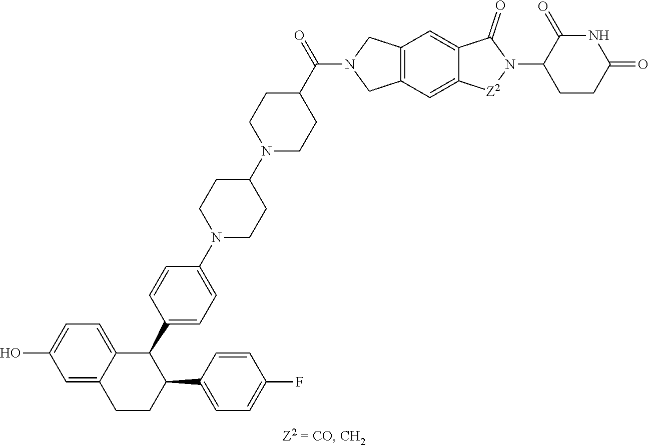

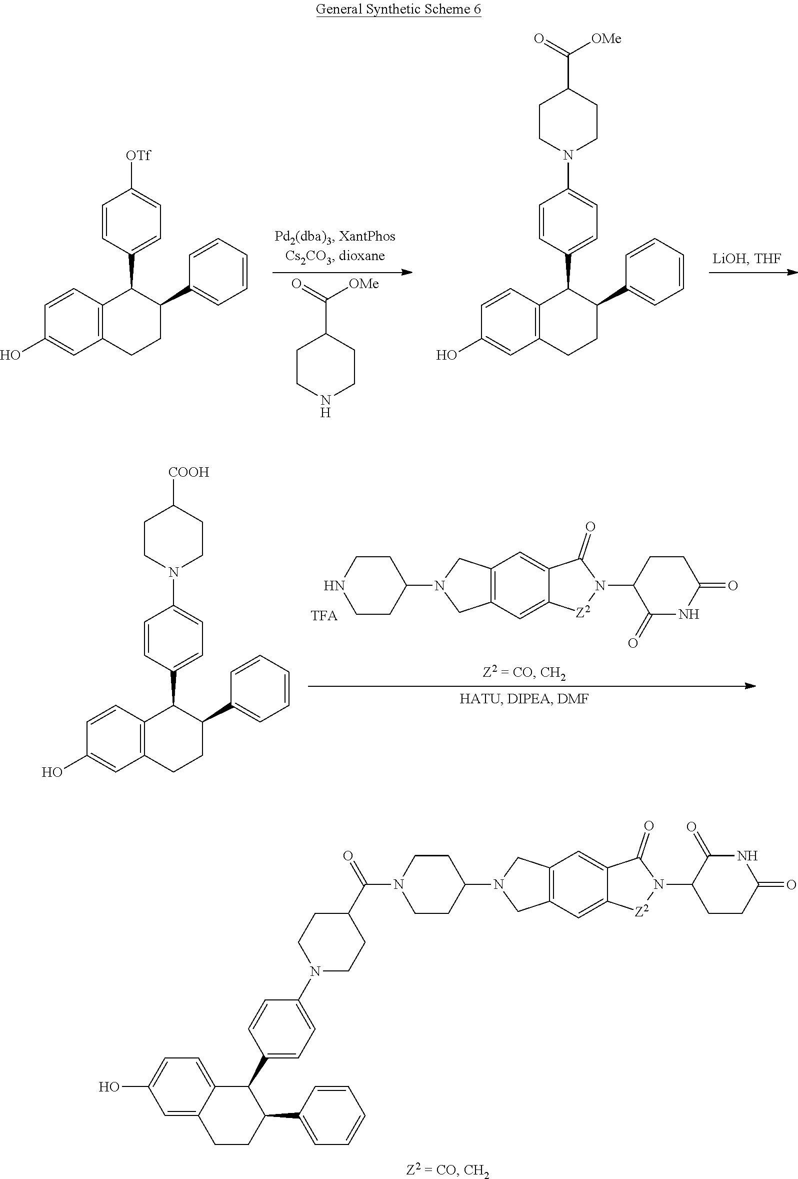

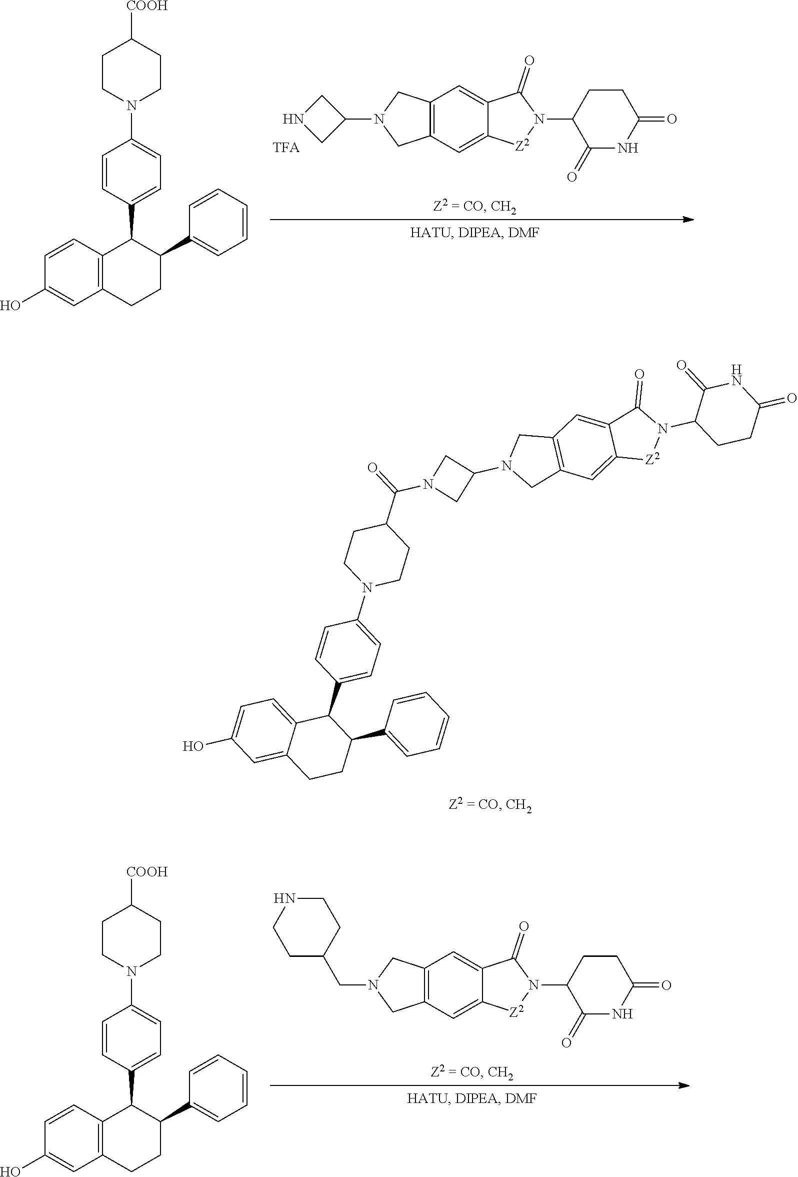

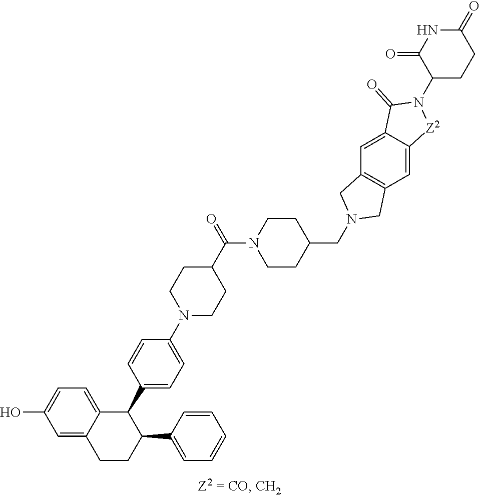

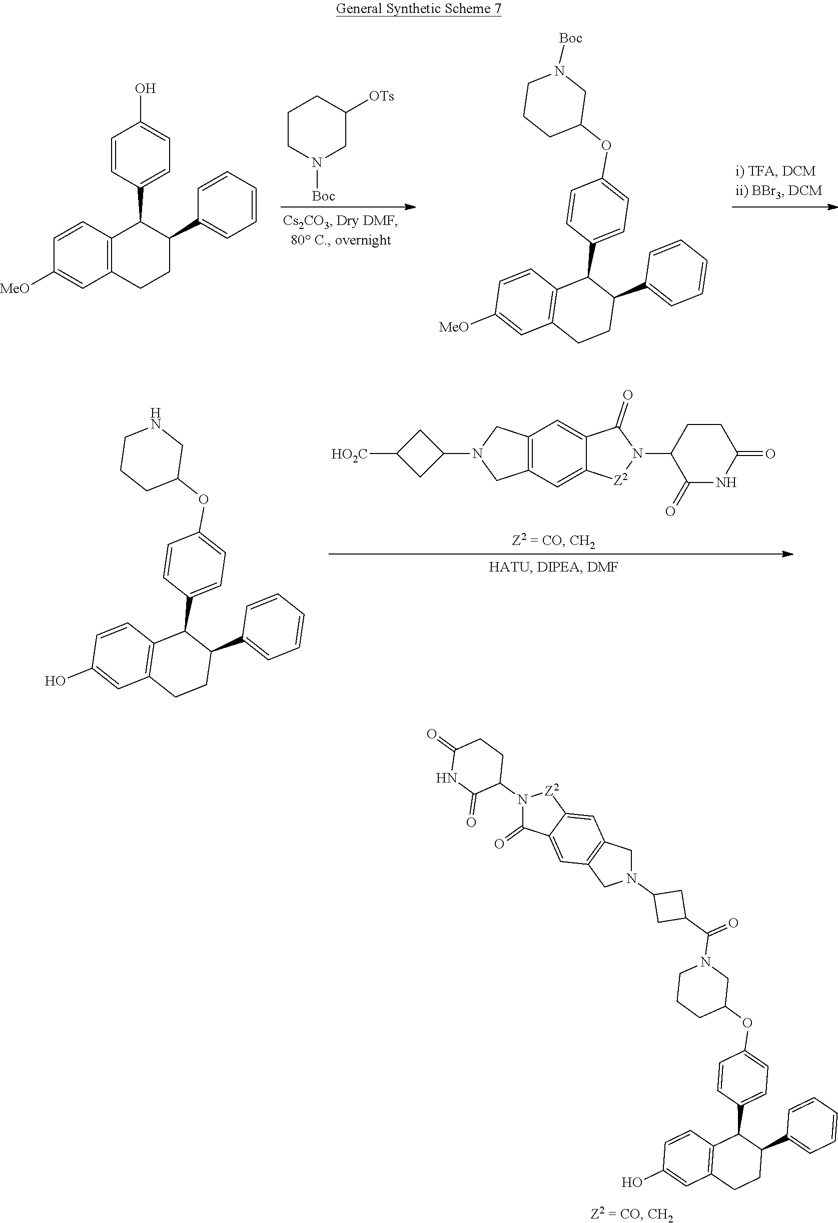

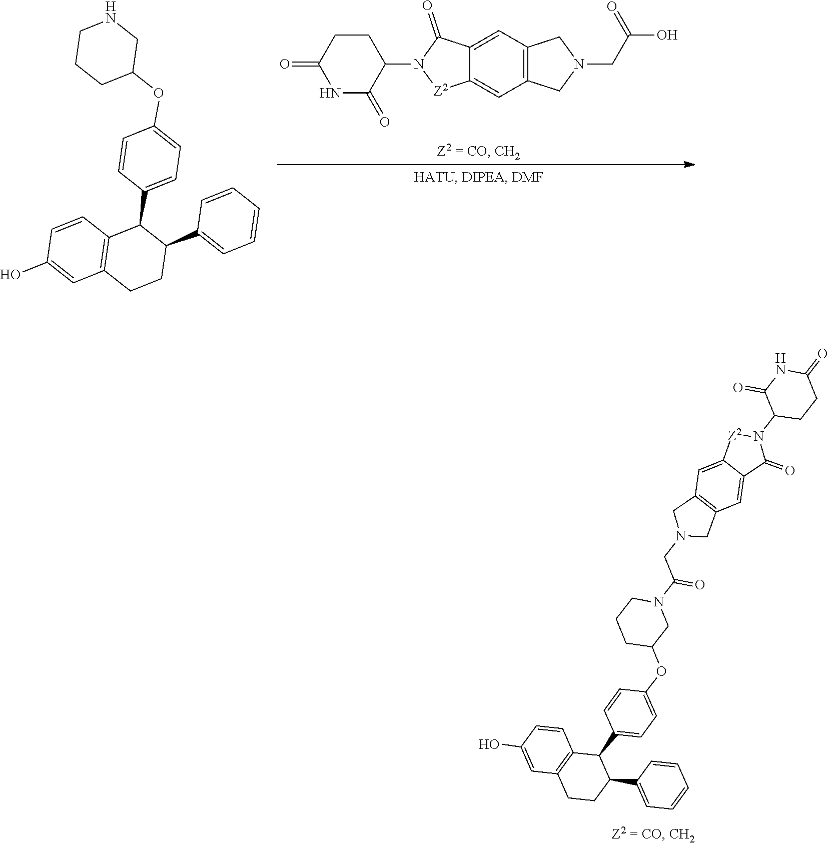

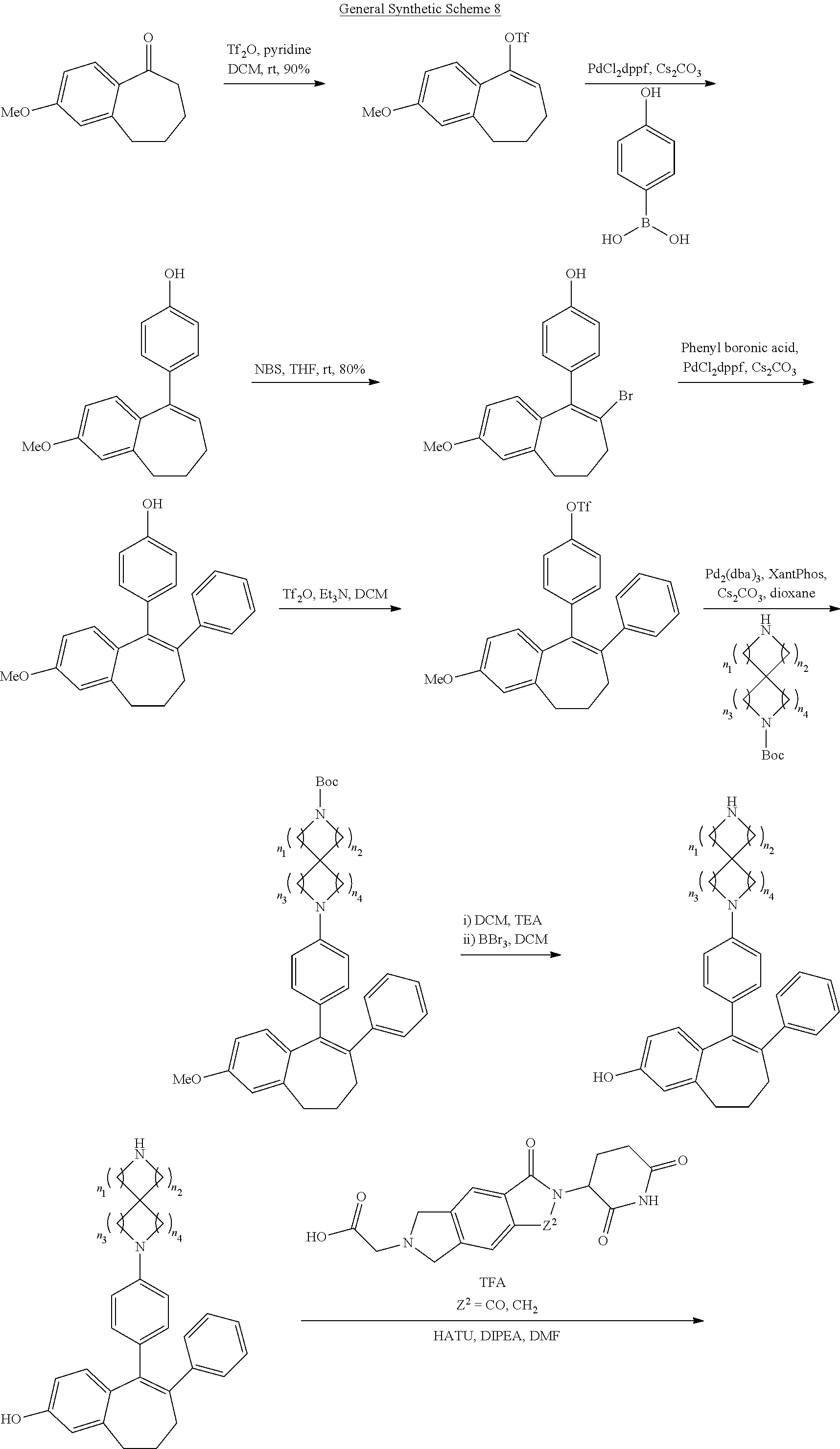

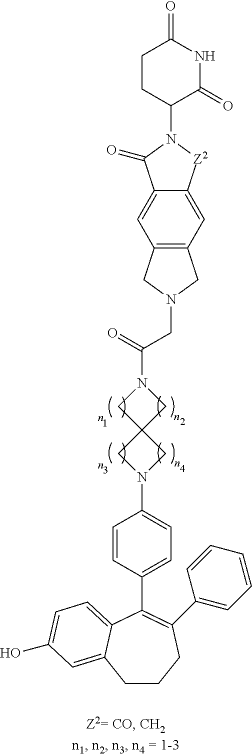

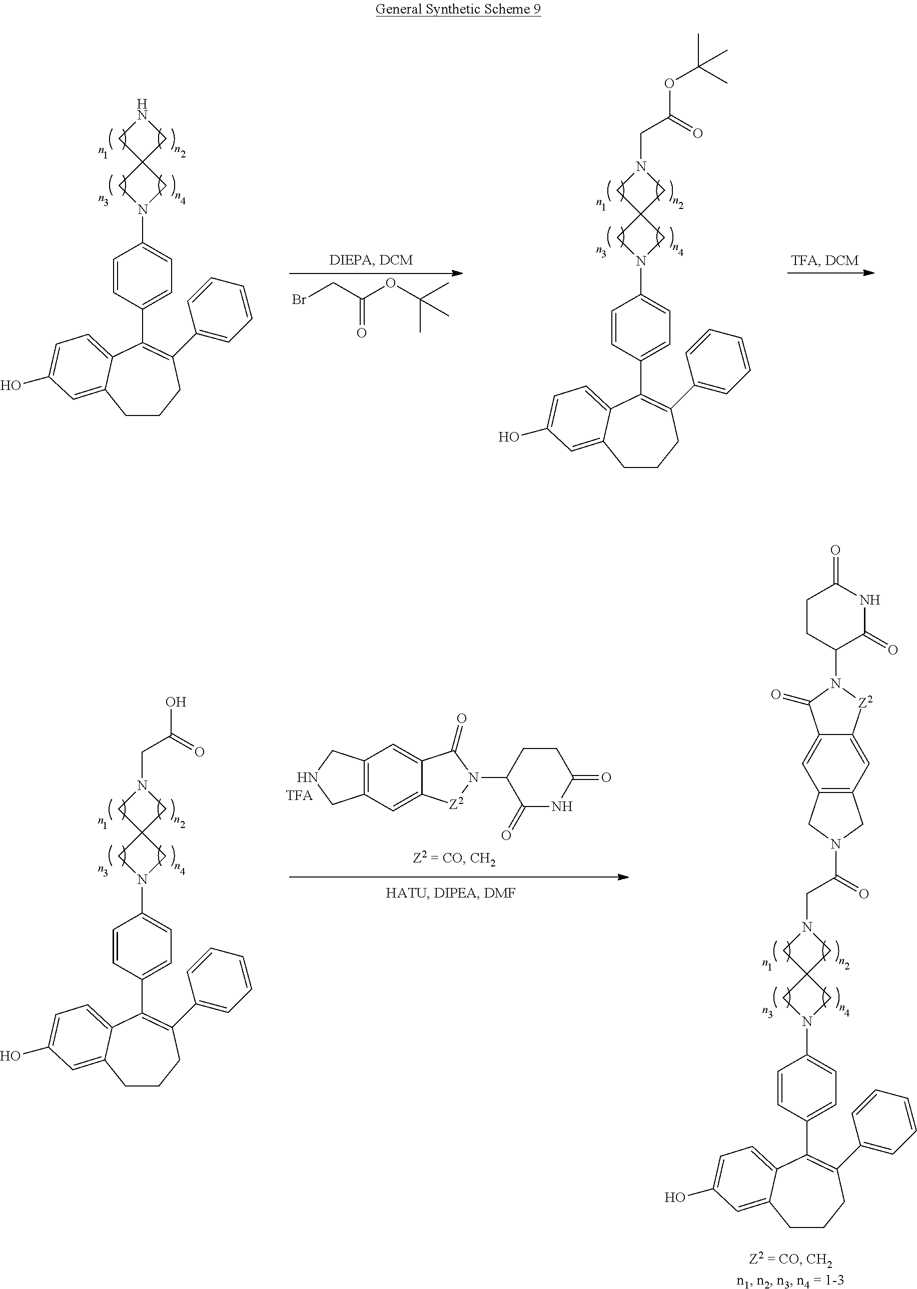

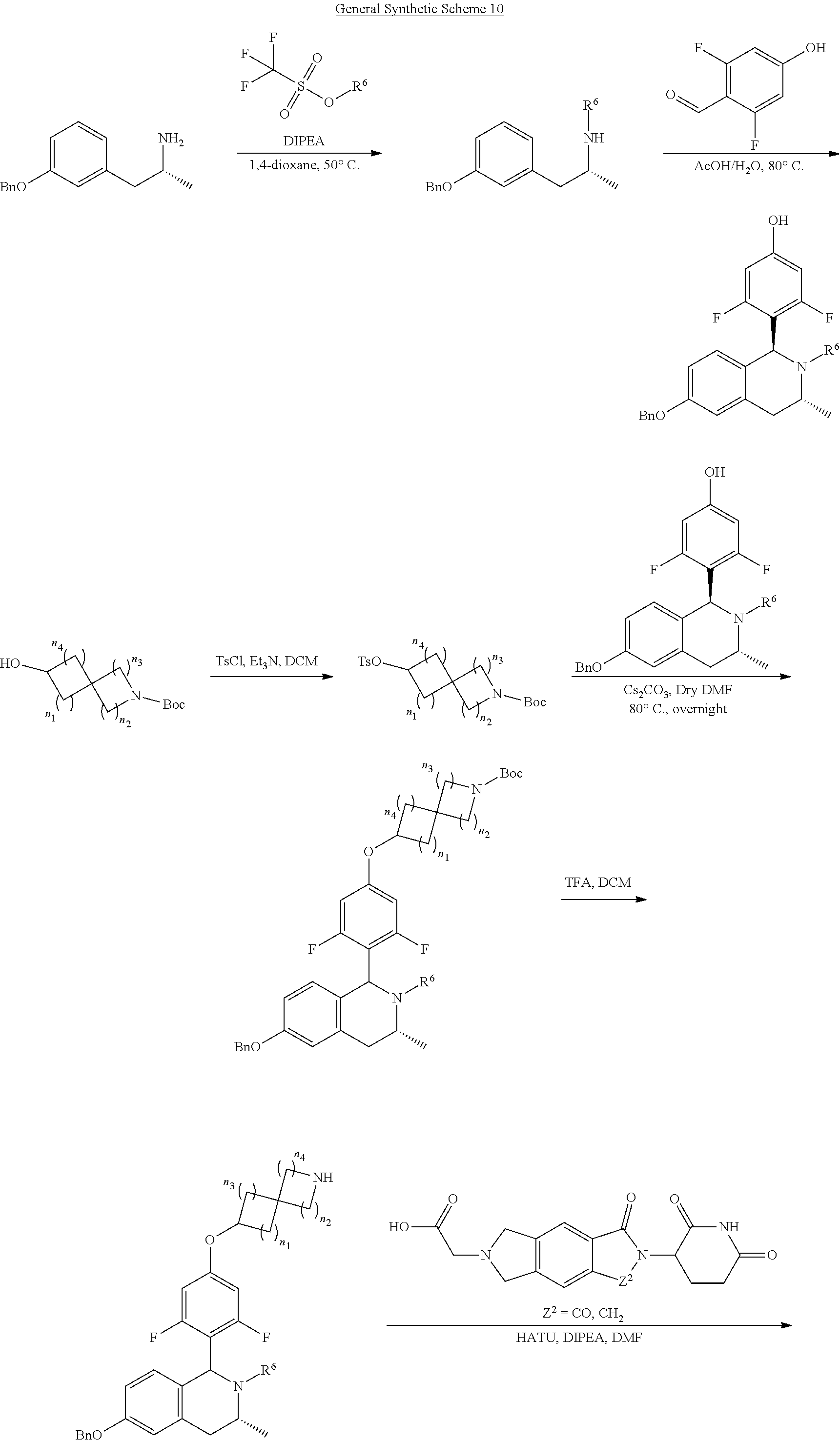

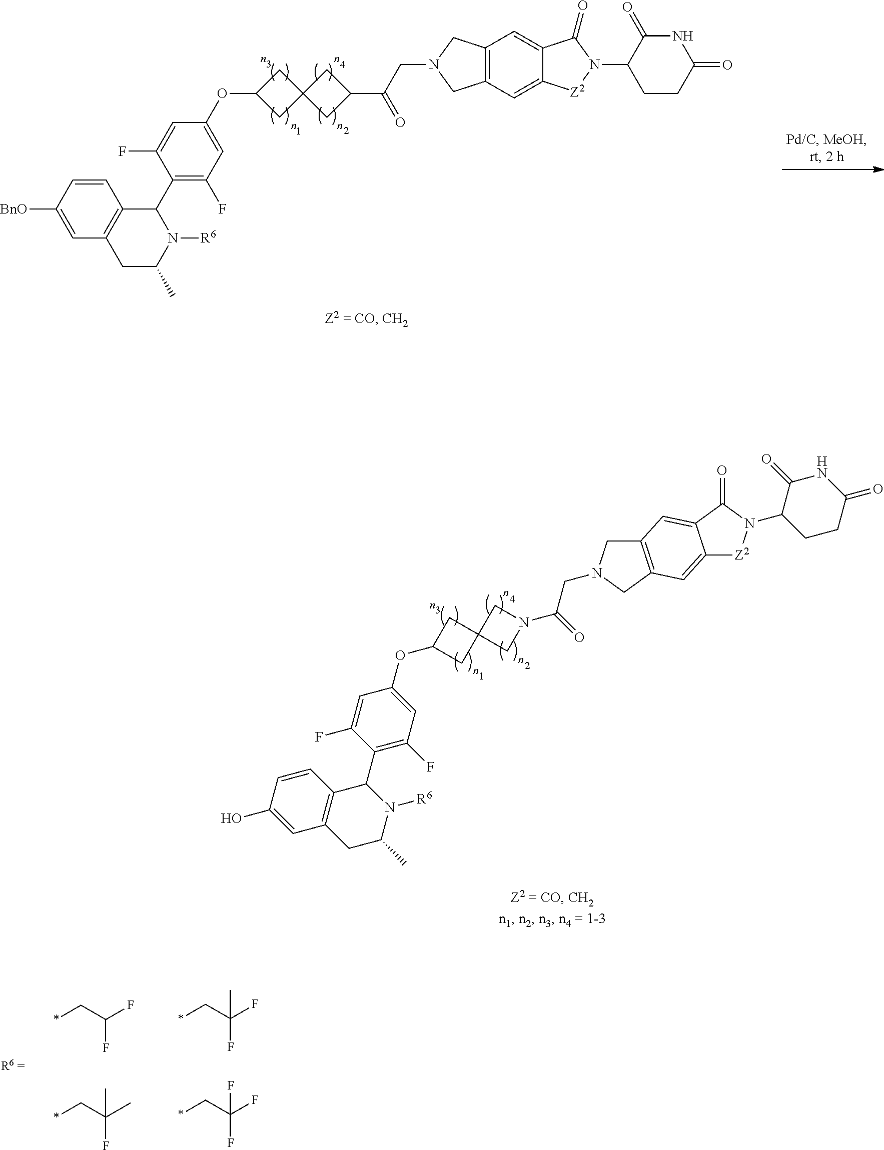

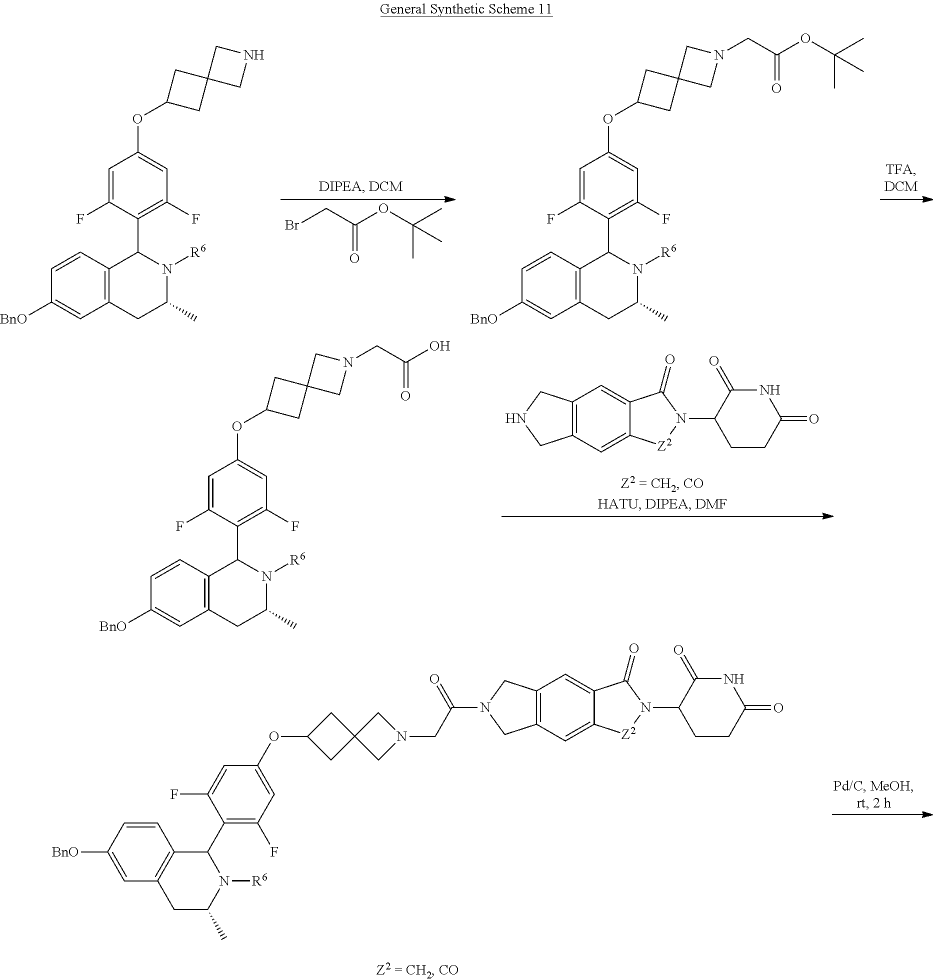

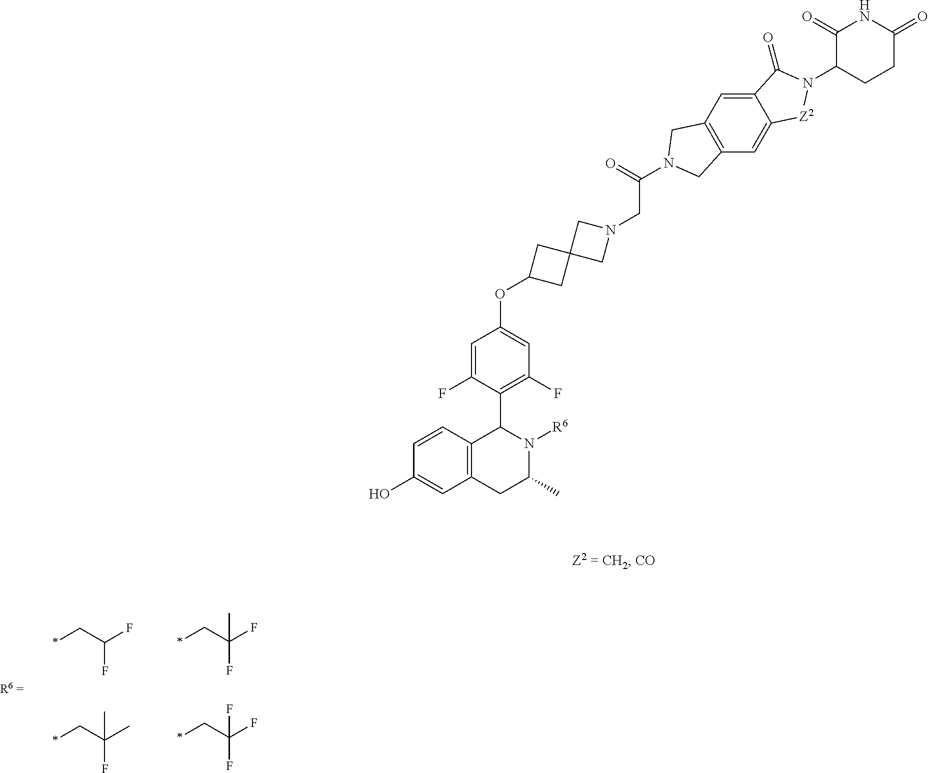

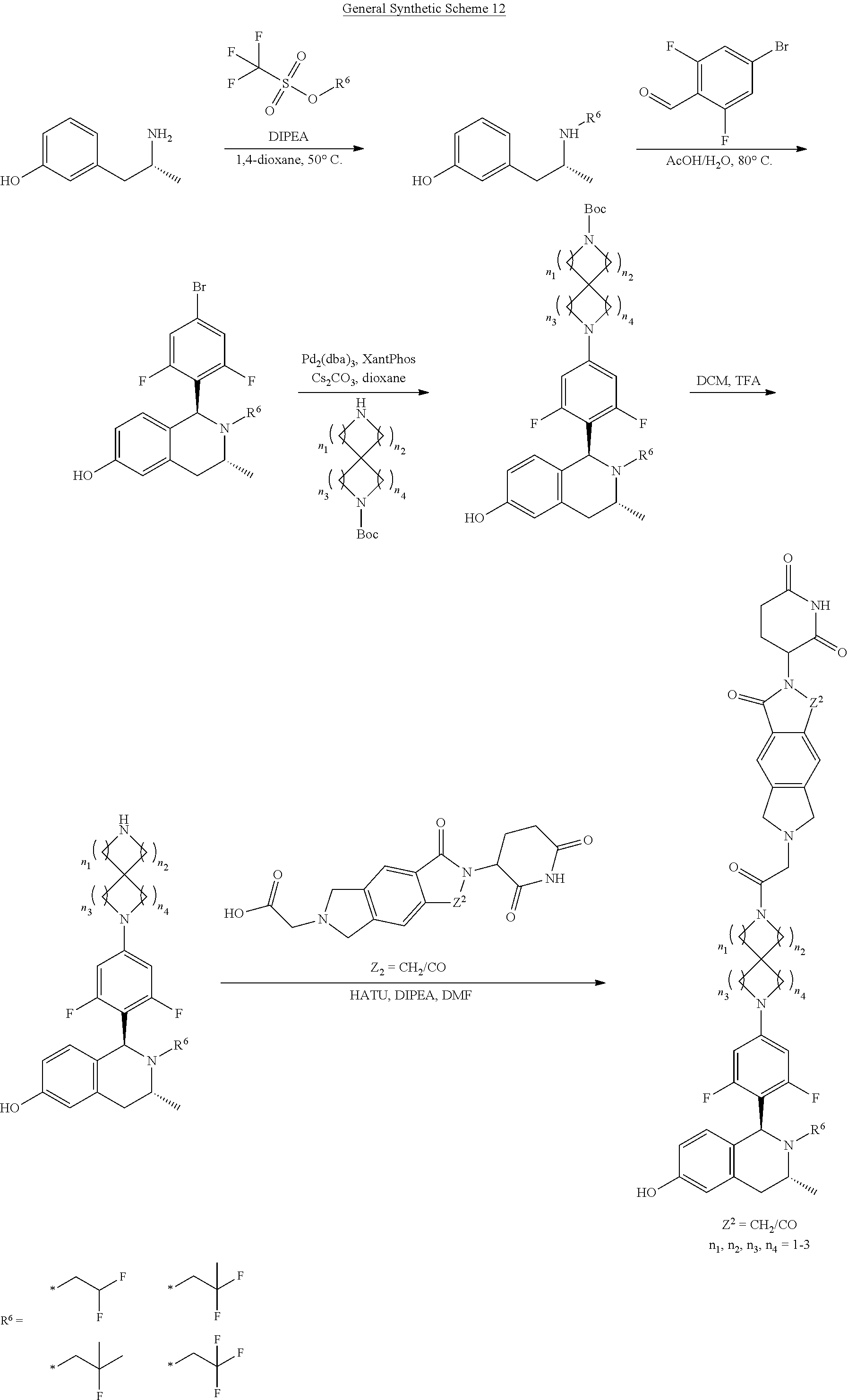

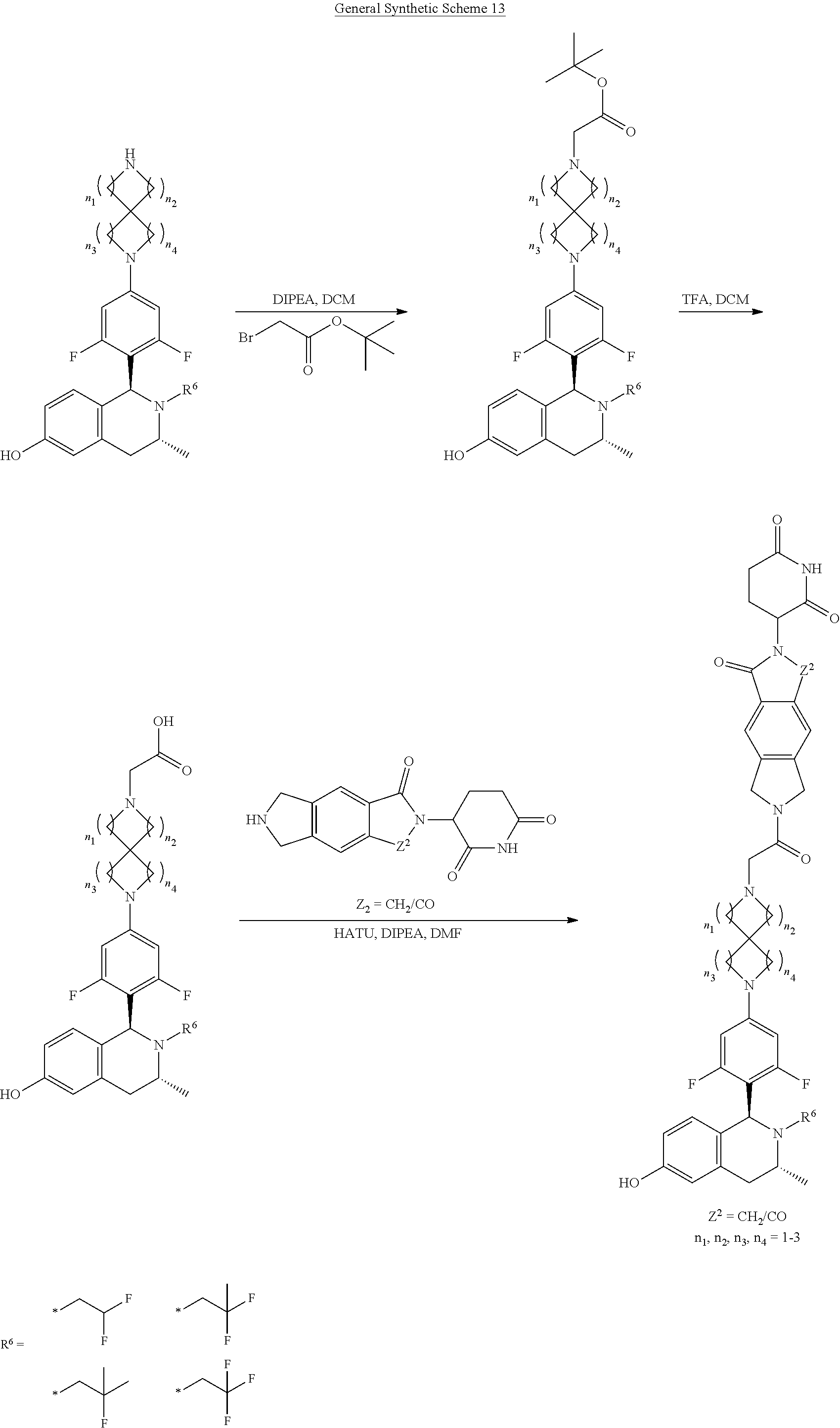

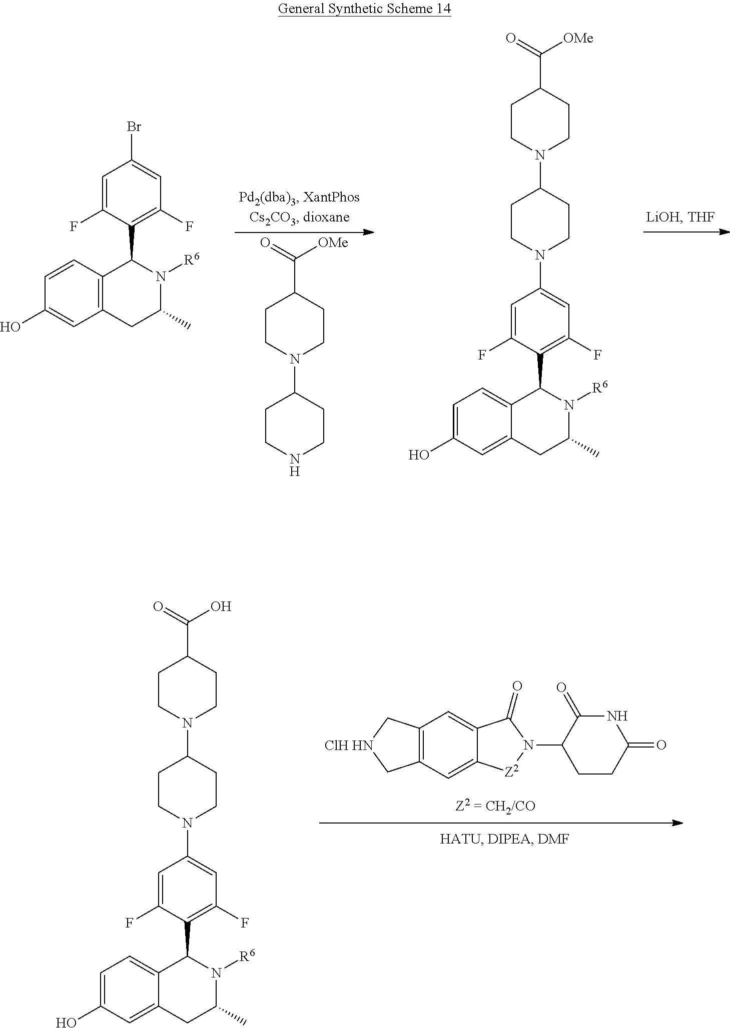

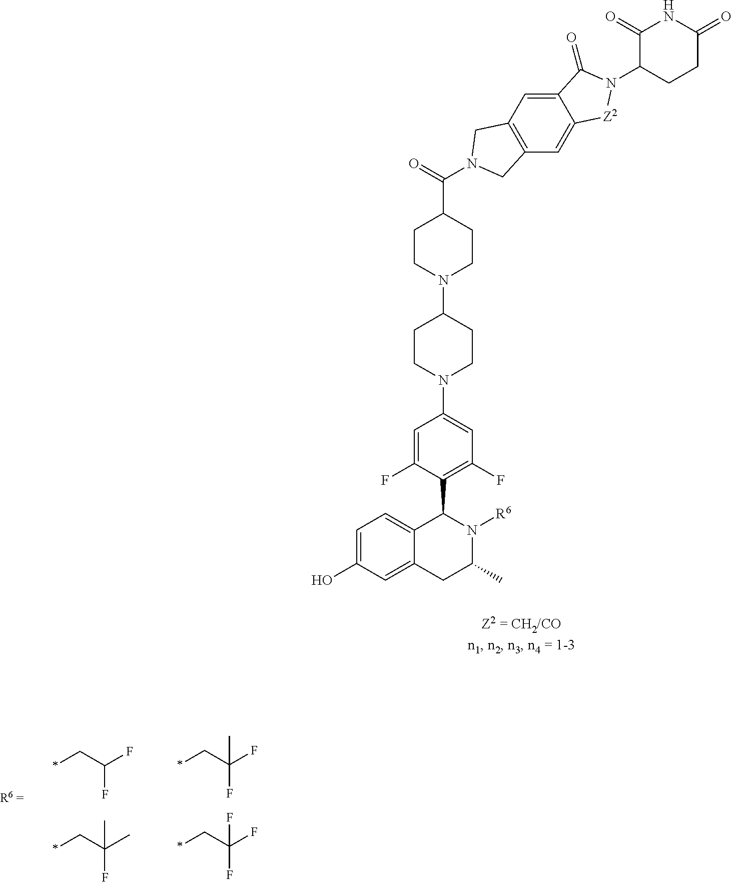

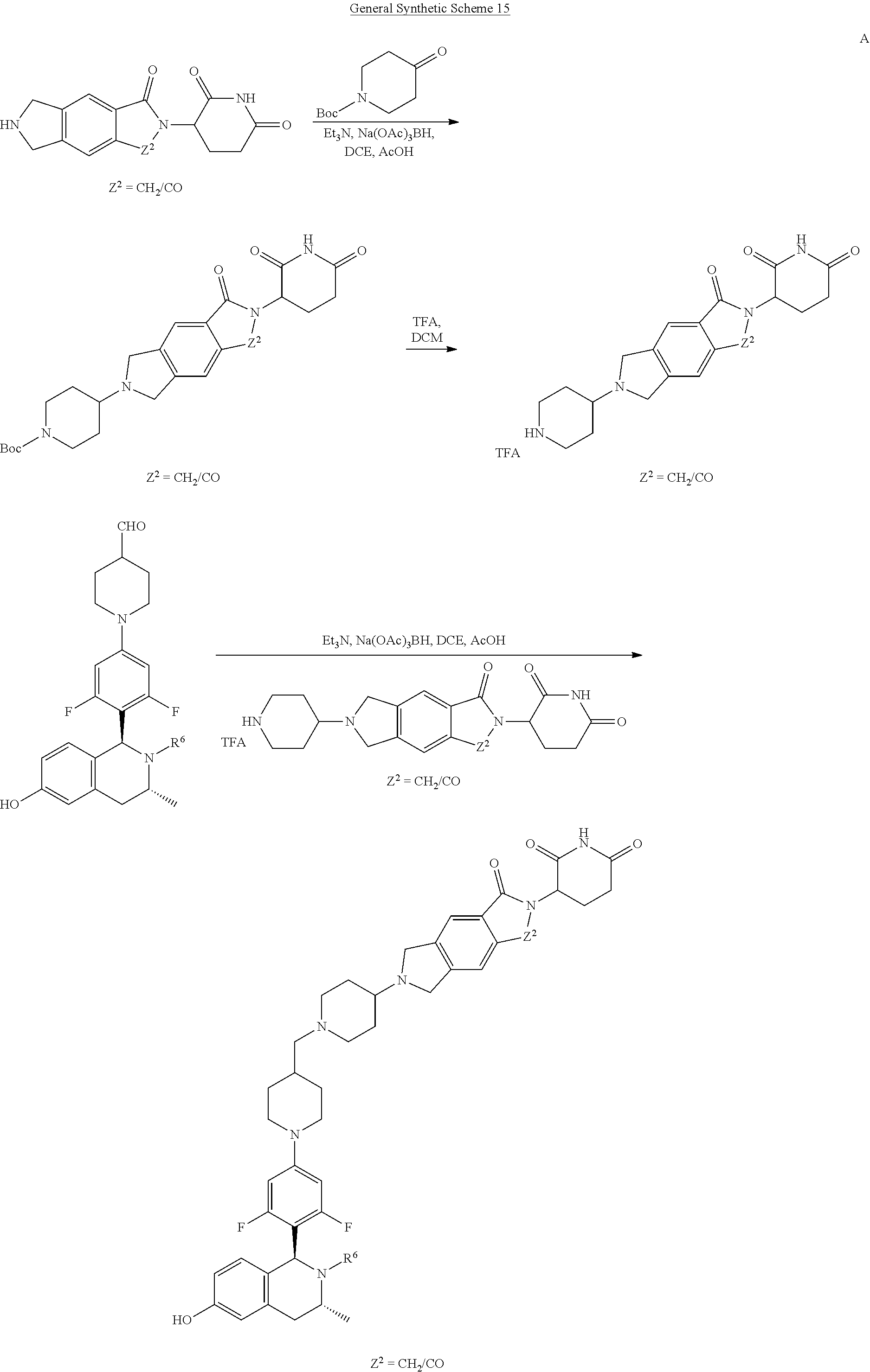

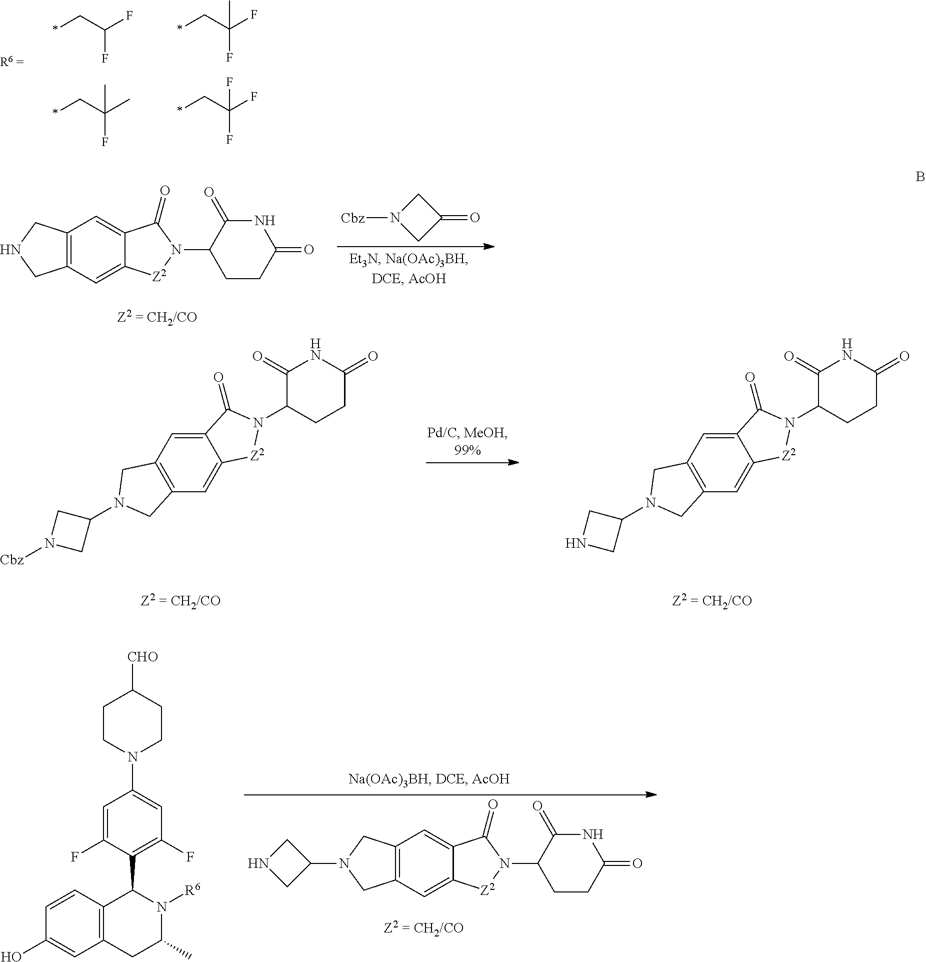

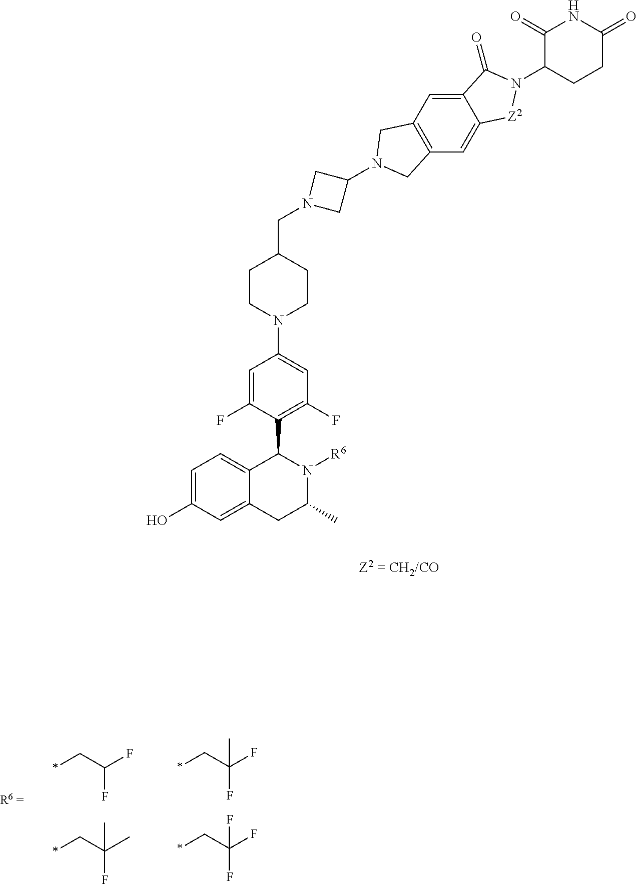

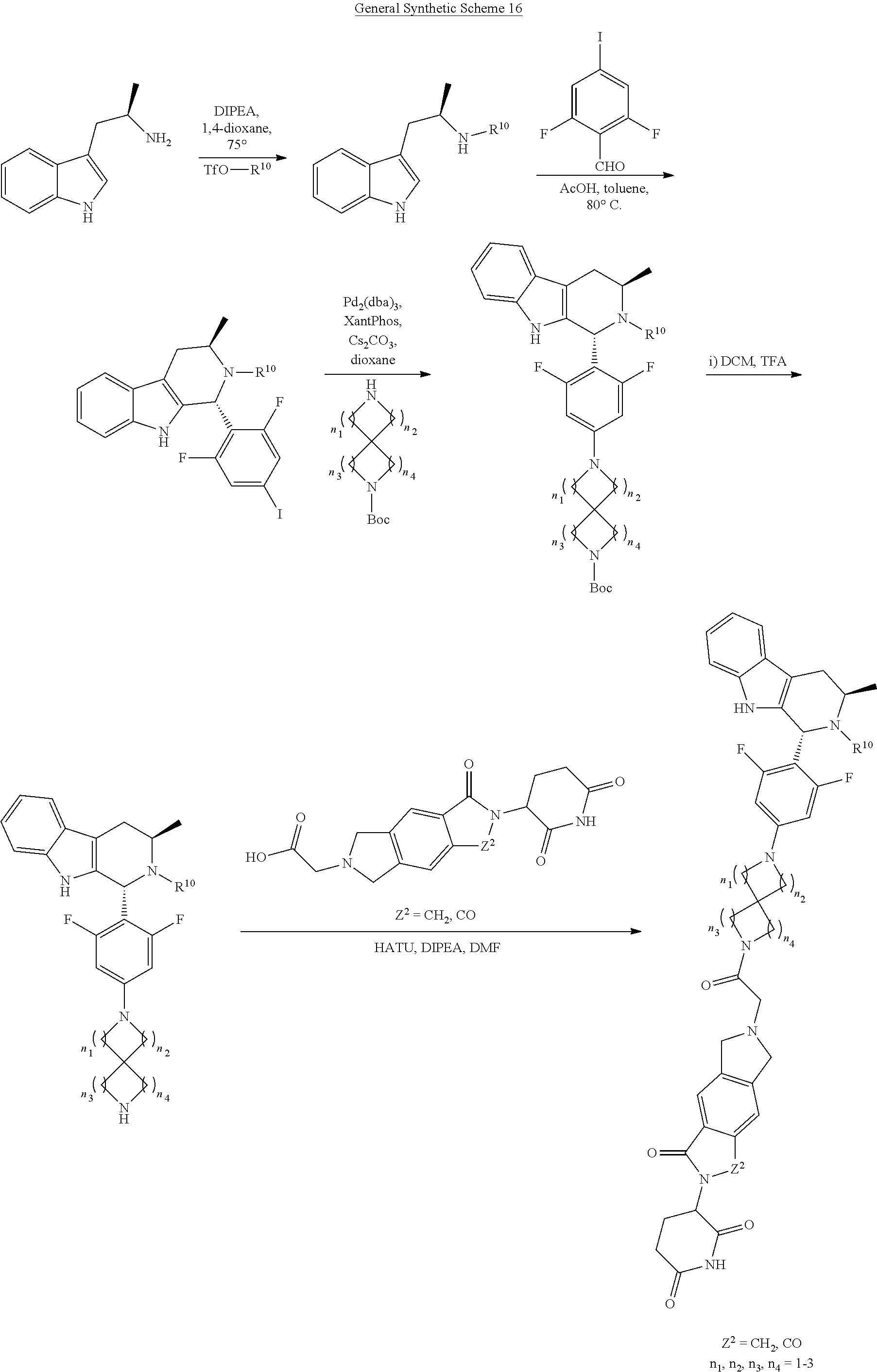

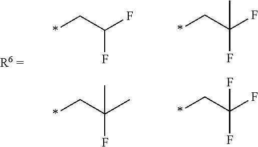

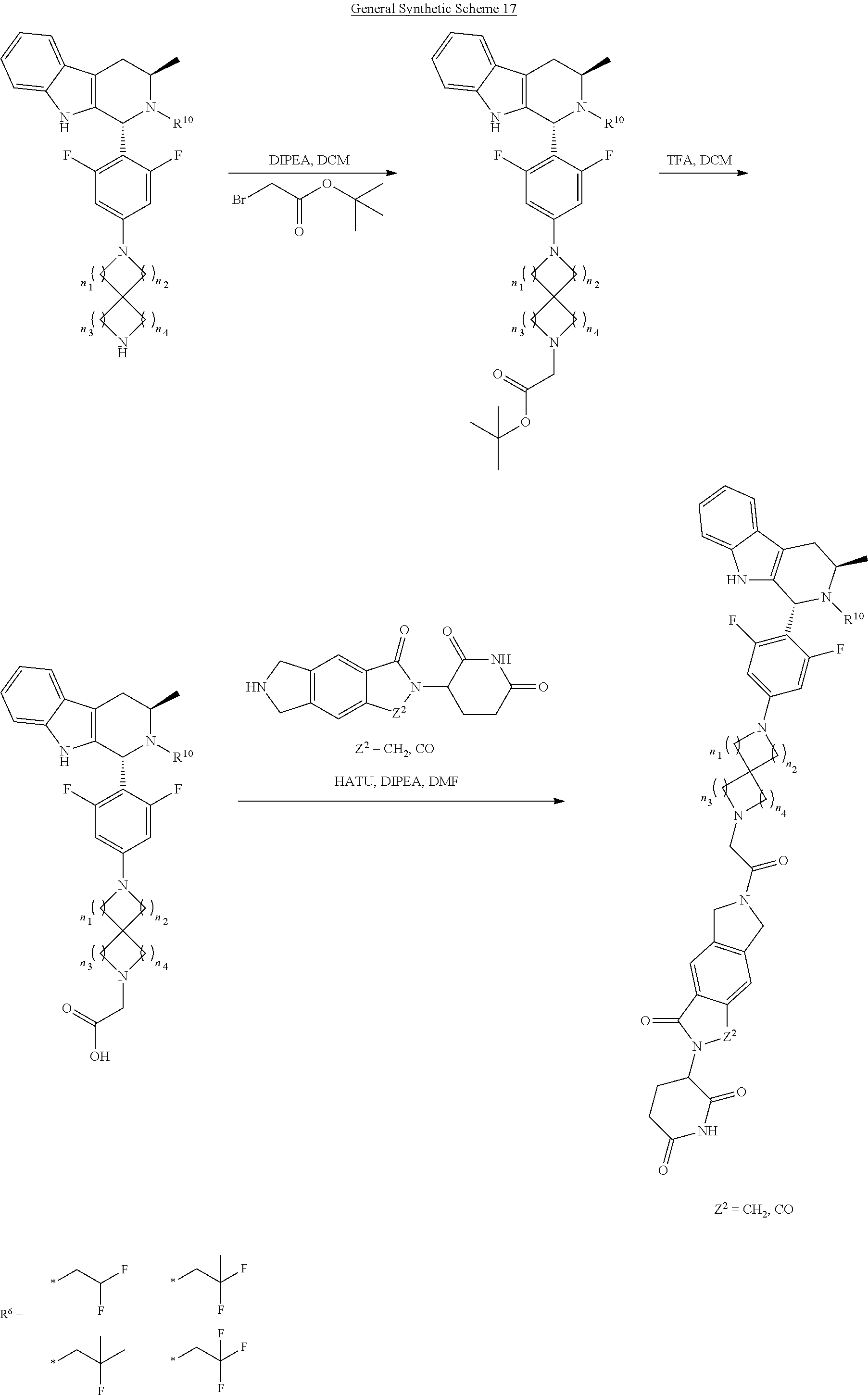

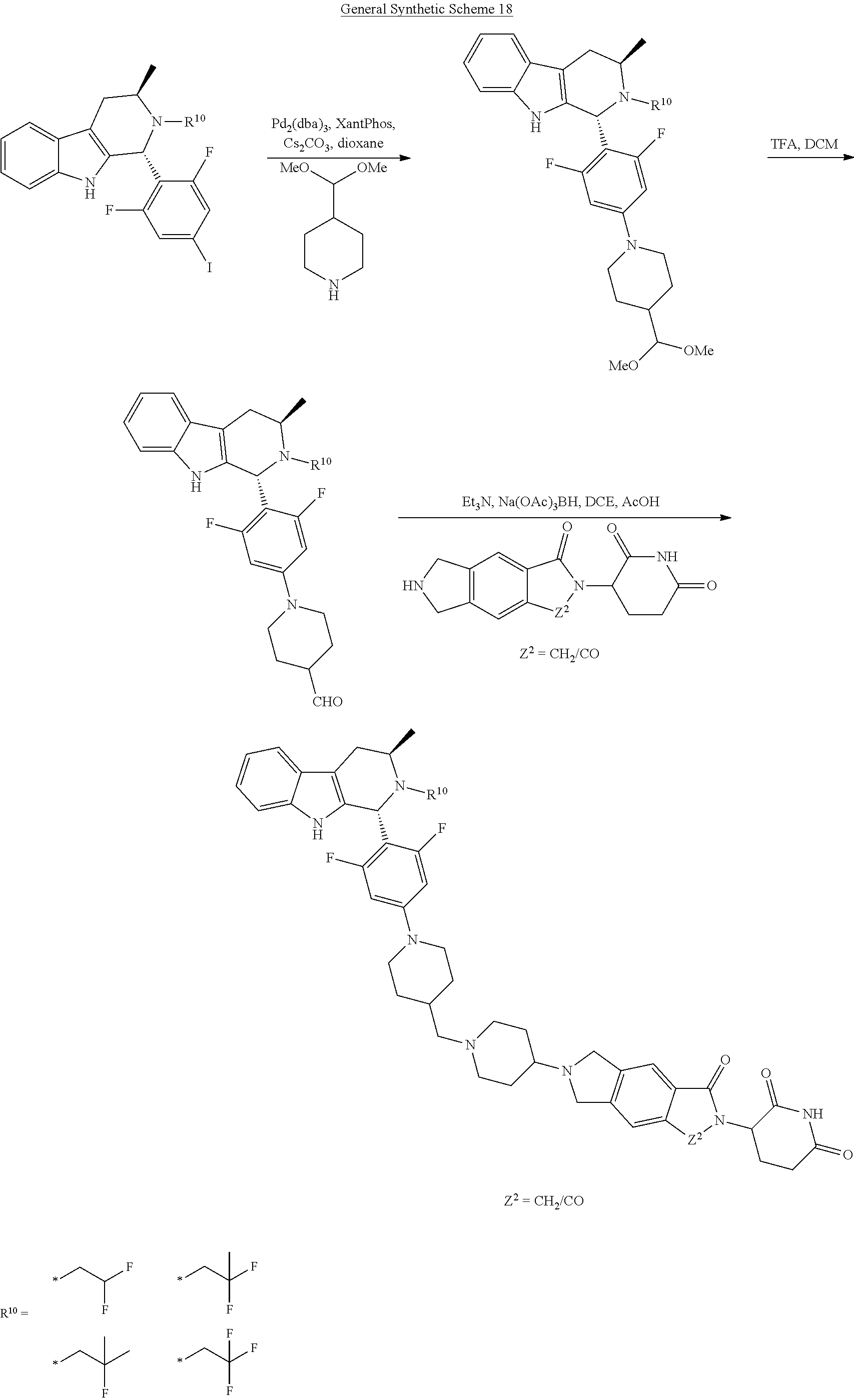

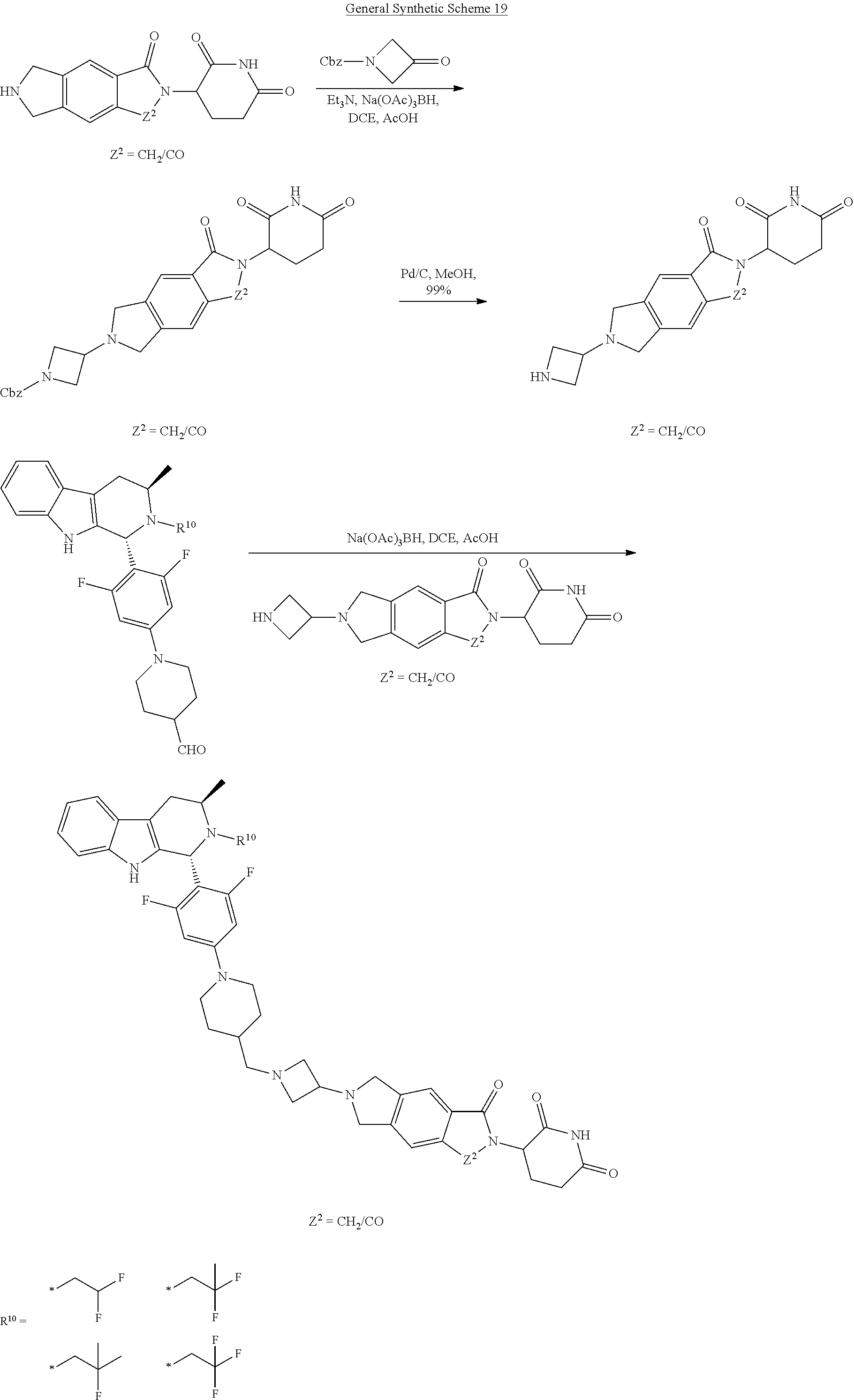

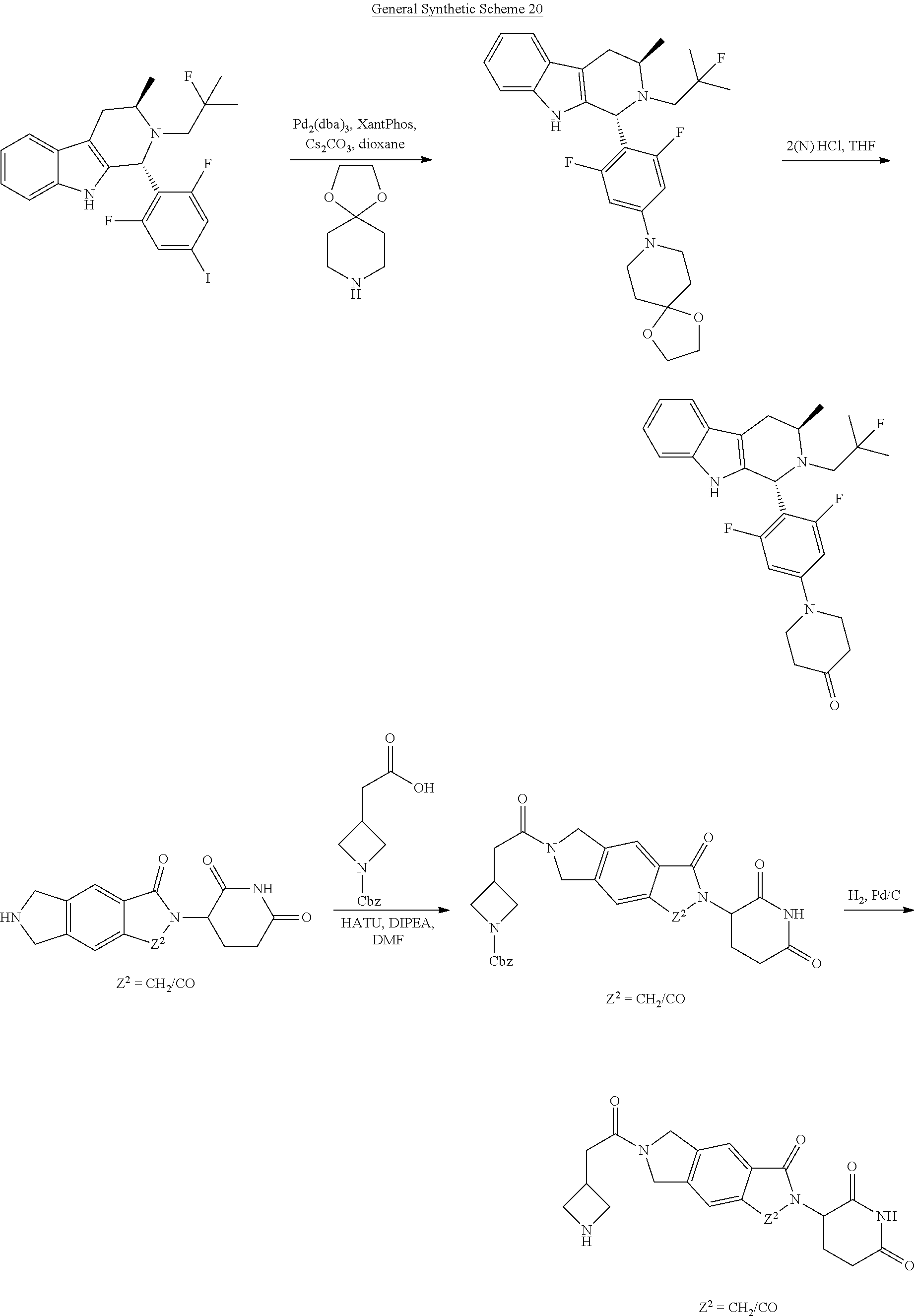

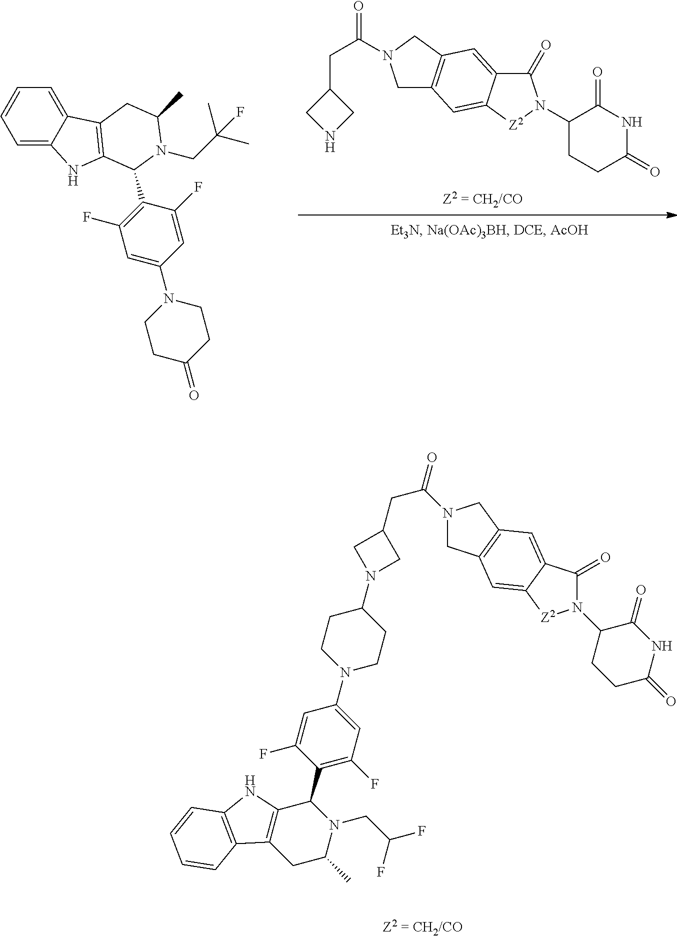

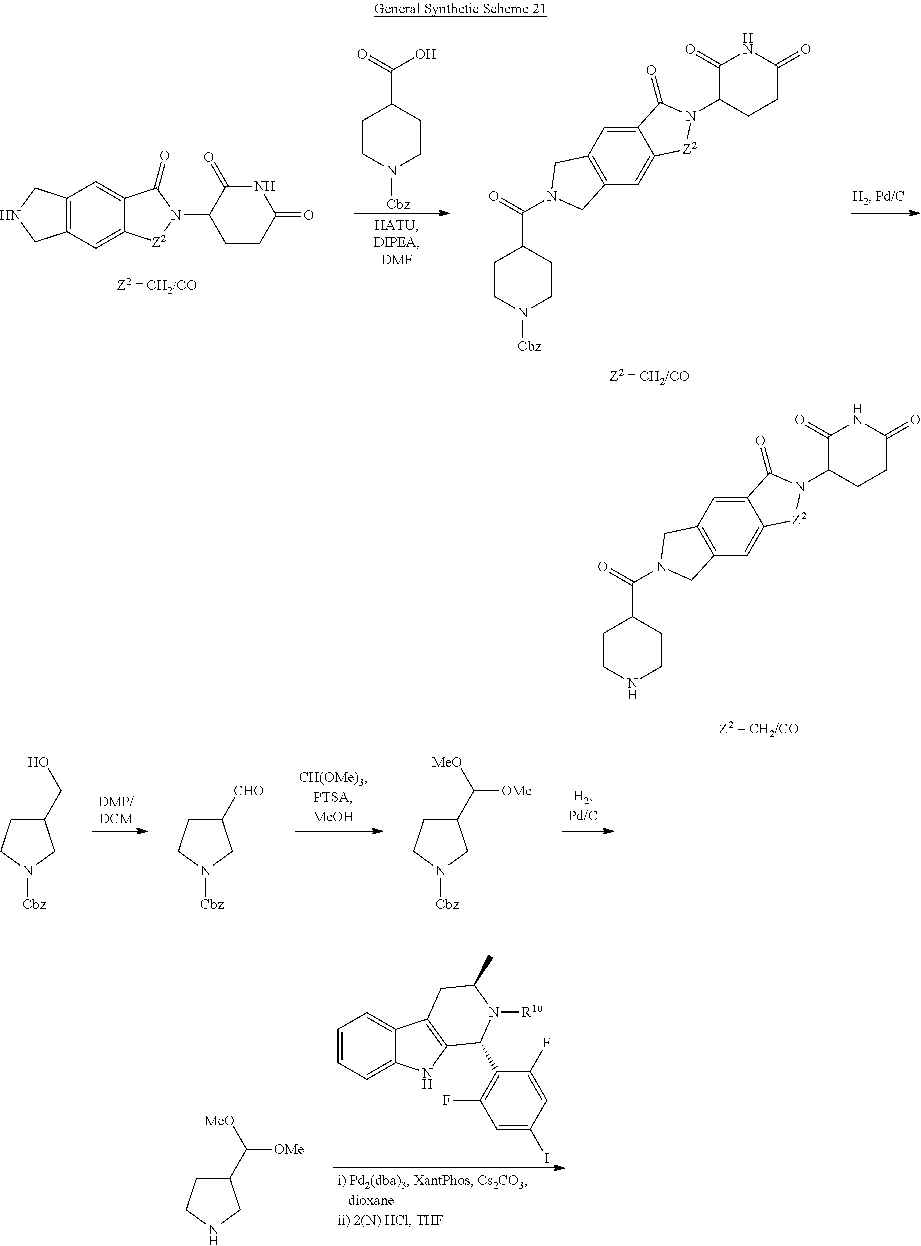

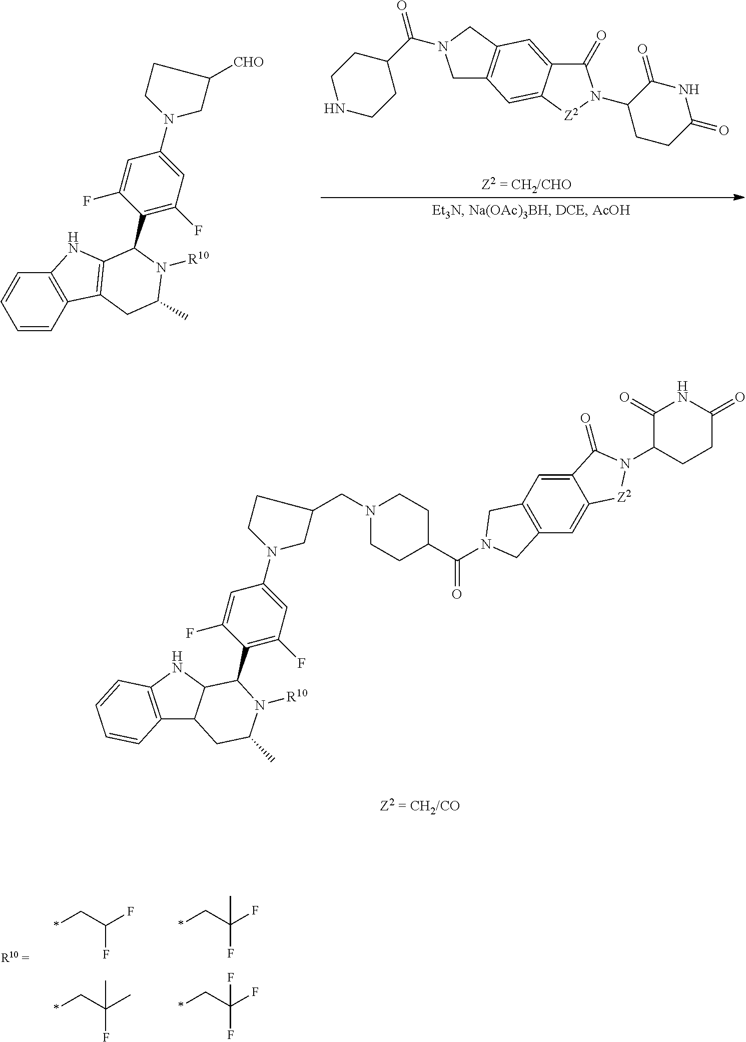

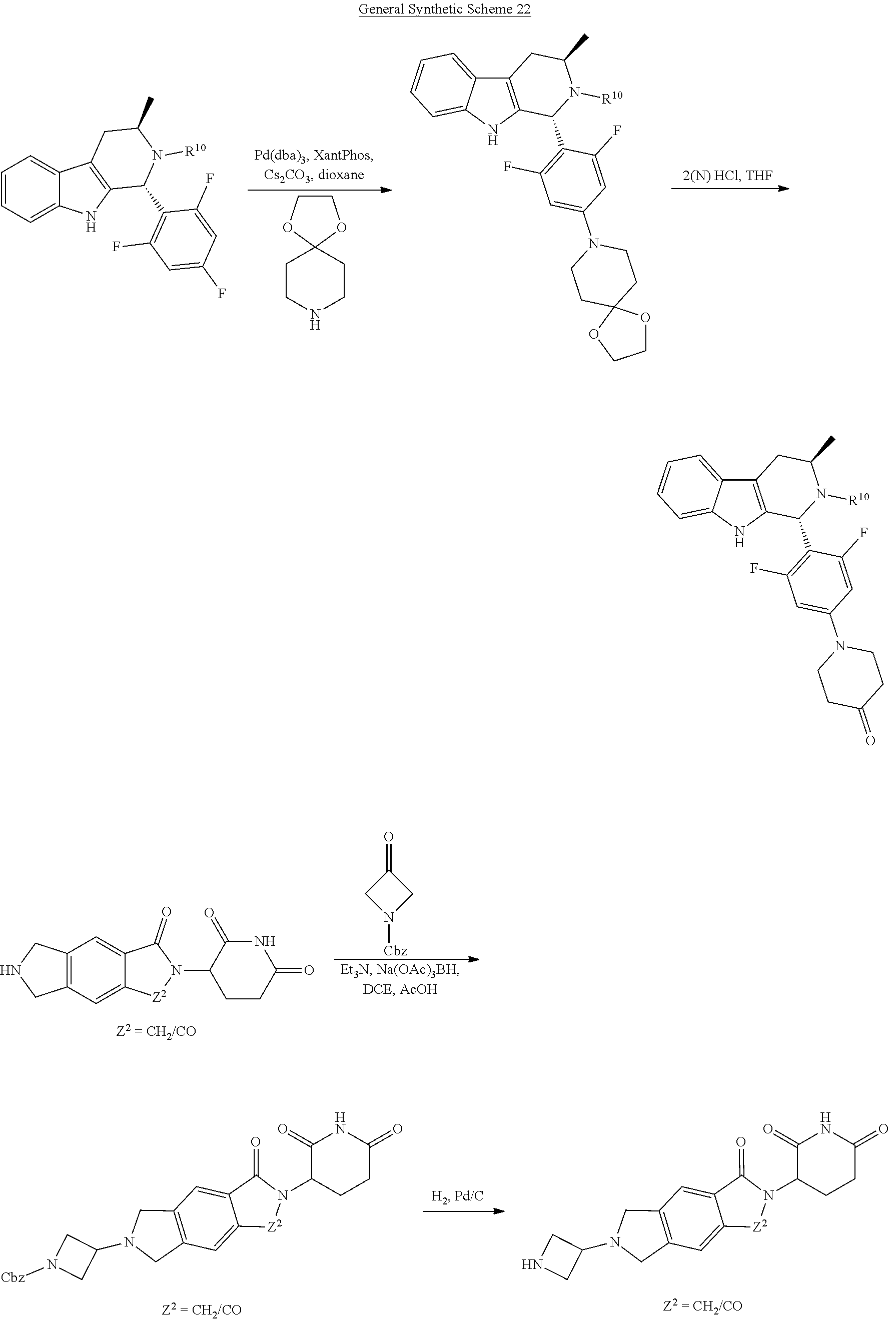

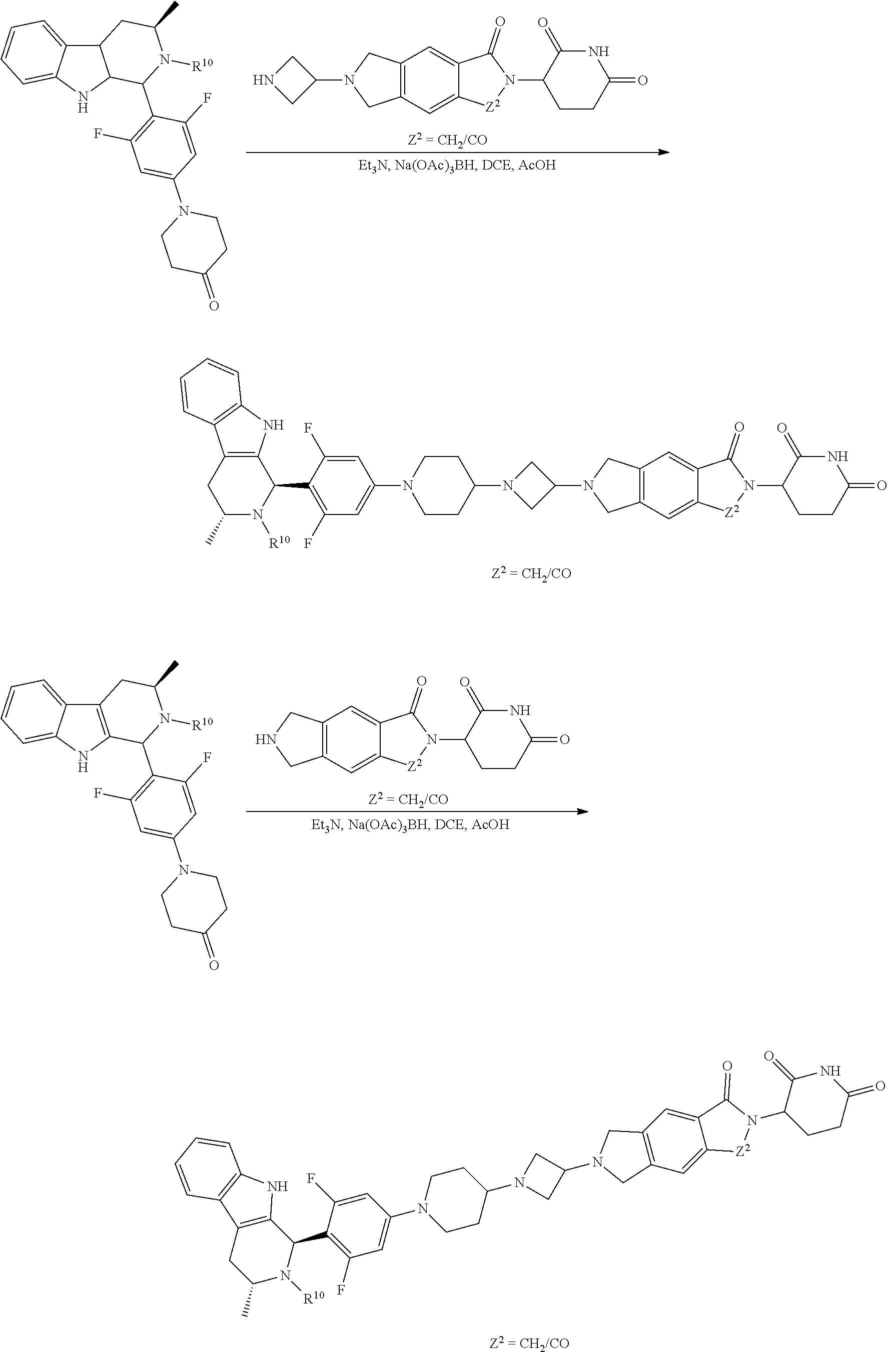

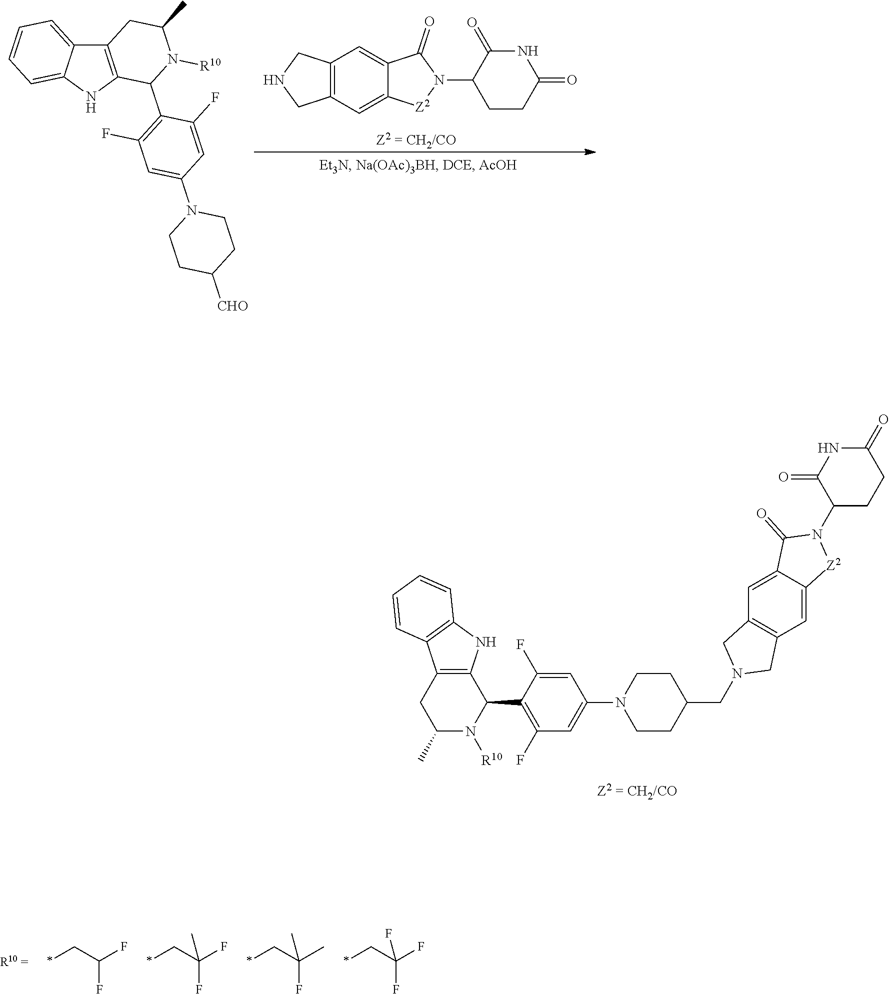

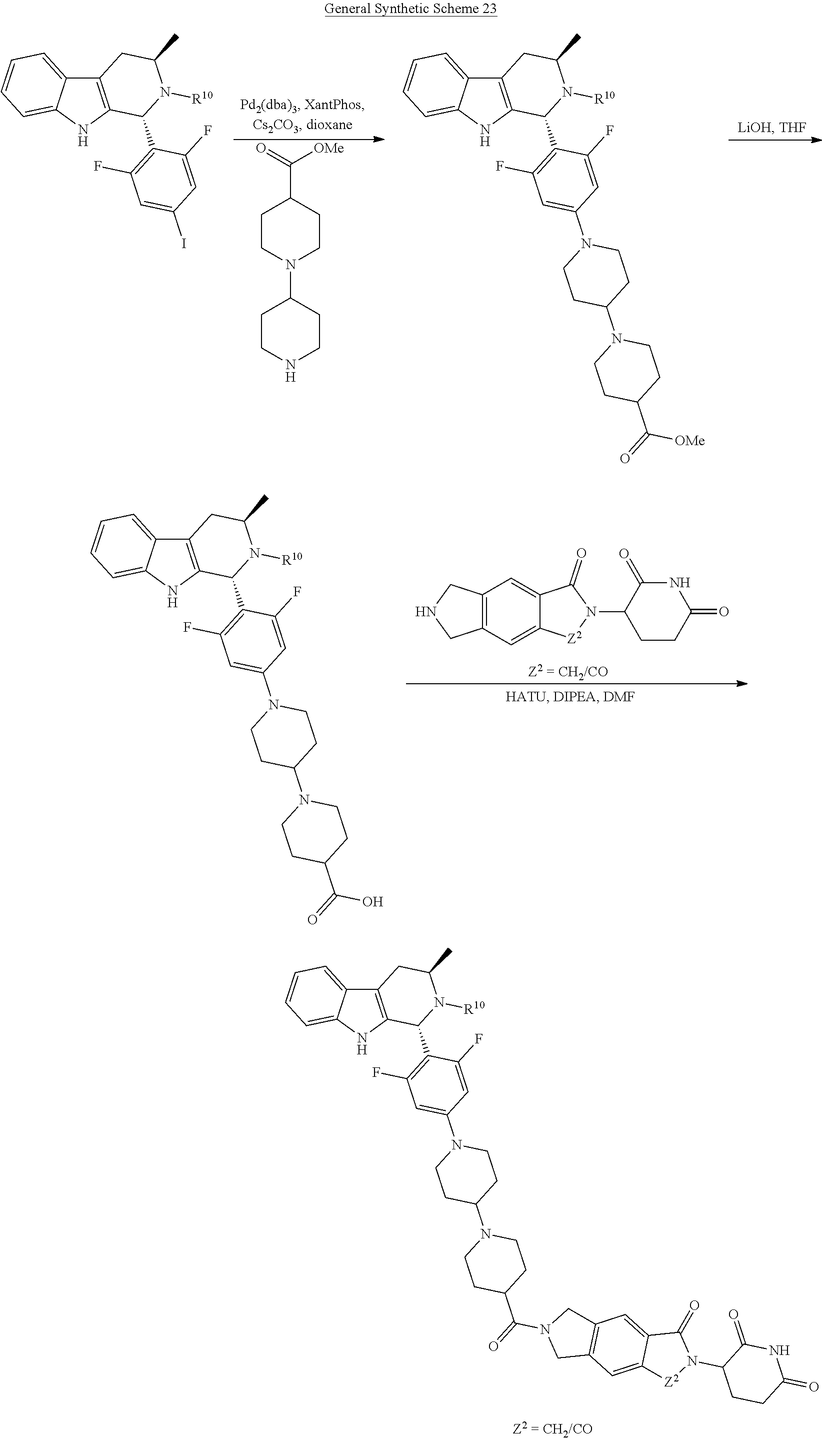

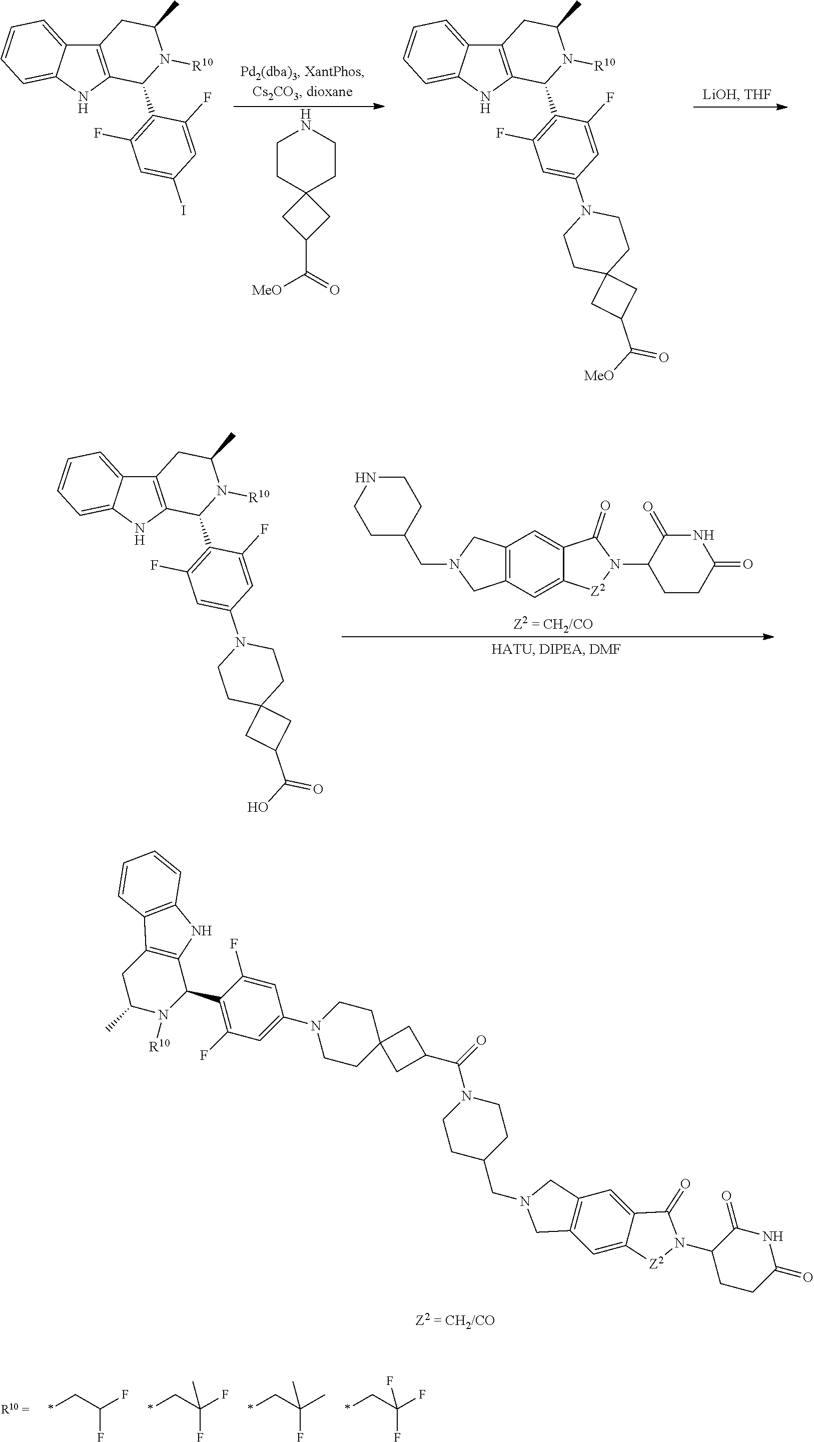

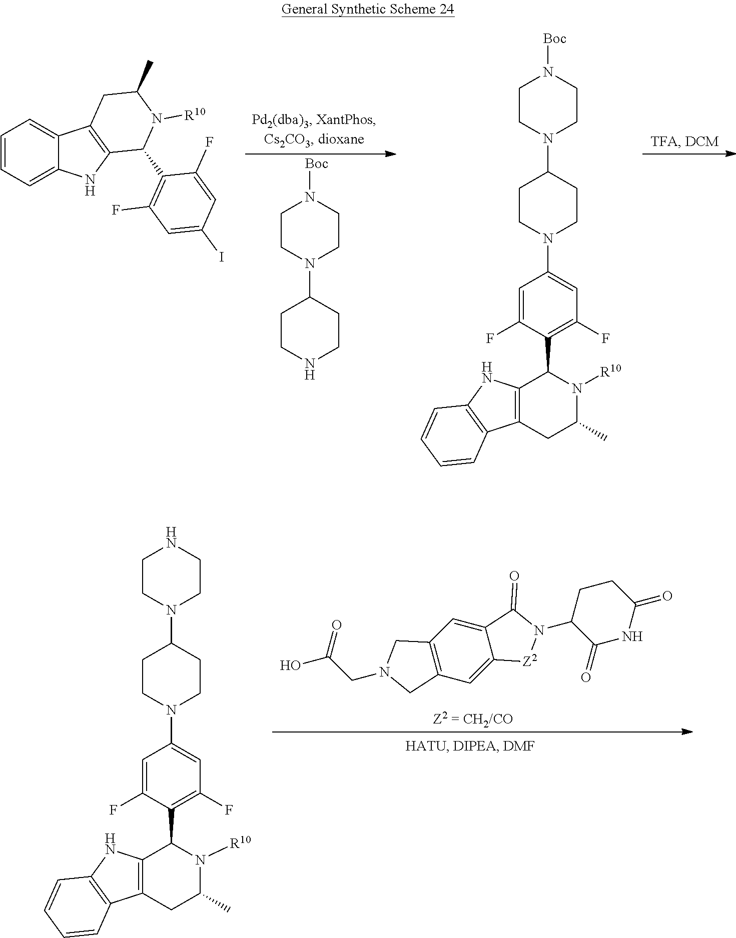

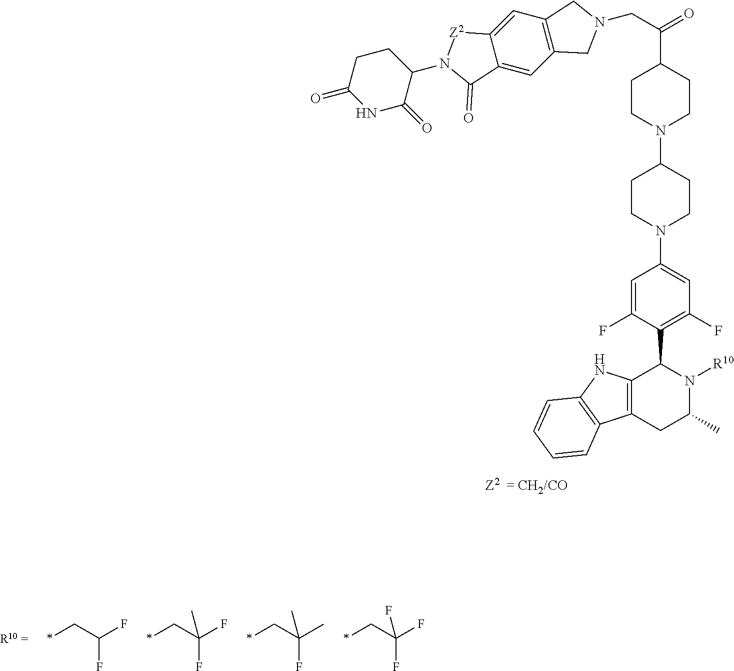

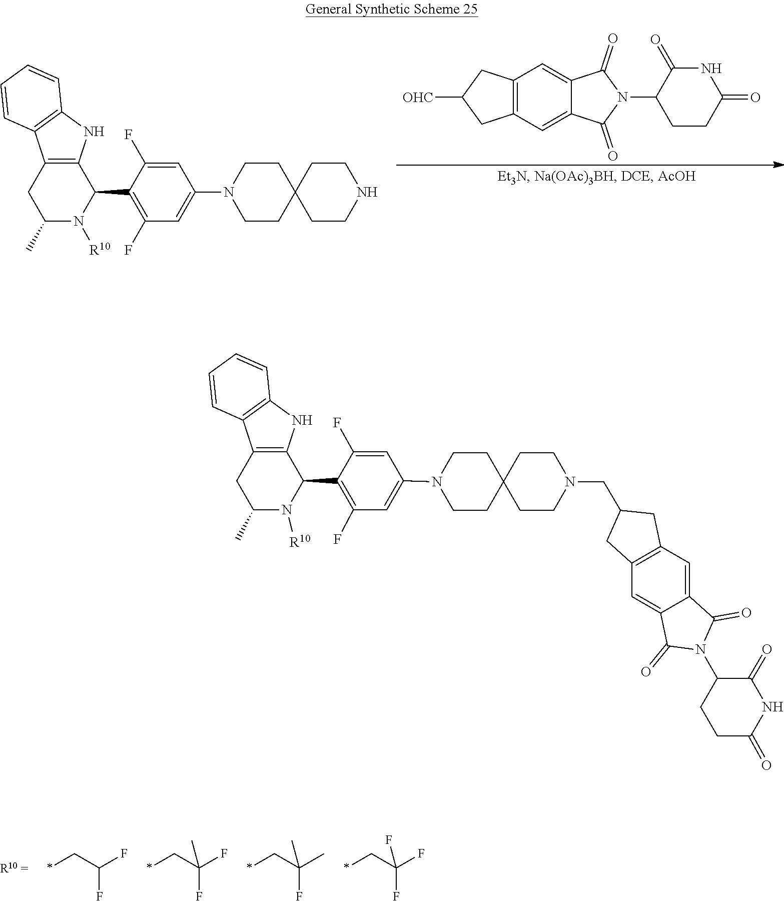

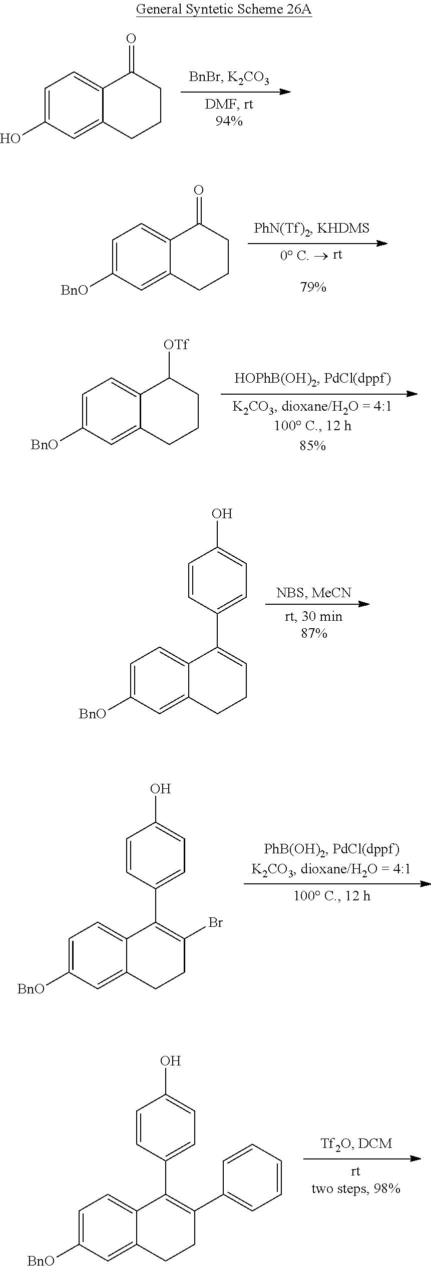 [SMILES: O=C1CCCc2cc(O)ccc21 ; O=C1CCCc2cc(OCc3ccccc3)ccc21 ; O=S(=O)(OC1CCCc2cc(OCc3ccccc3)ccc21)C(F)(F)F ; Oc1ccc(C2=CCCc3cc(OCc4ccccc4)ccc32)cc1 ; Oc1ccc(C2=C(Br)CCc3cc(OCc4ccccc4)ccc32)cc1 ; Oc1ccc(C2=C(c3ccccc3)CCc3cc(OCc4ccccc4)ccc32)cc1]

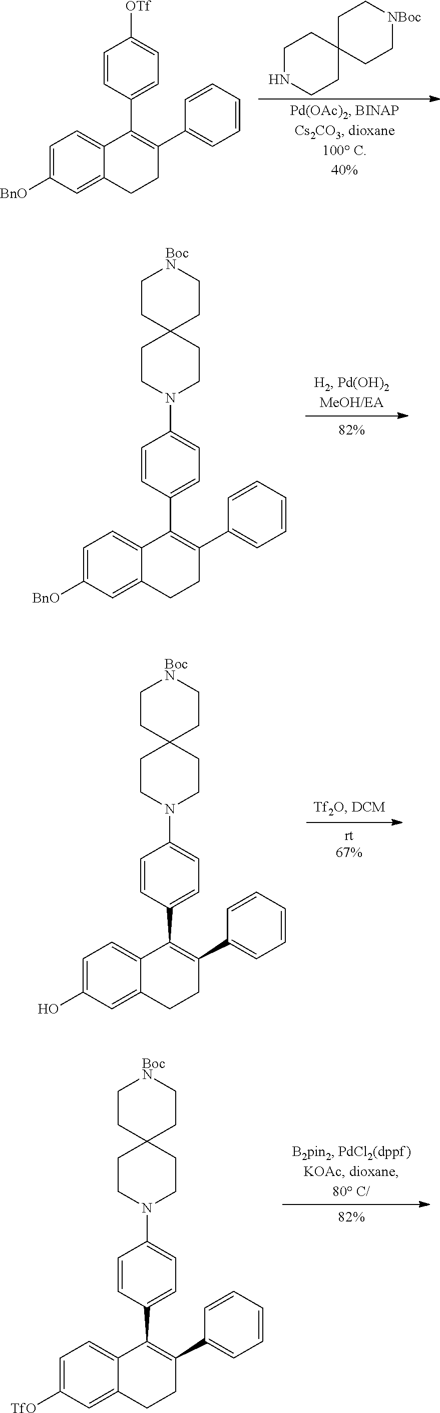

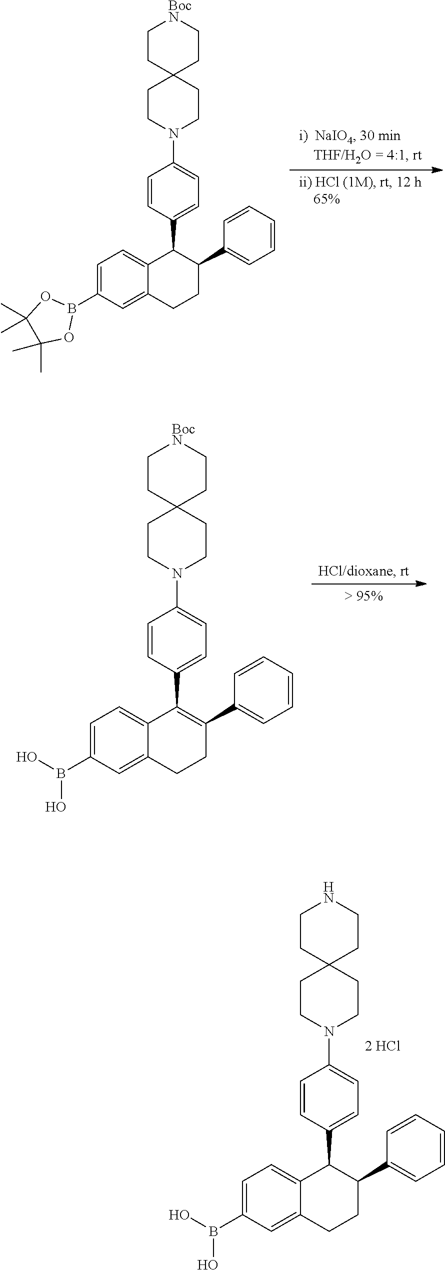

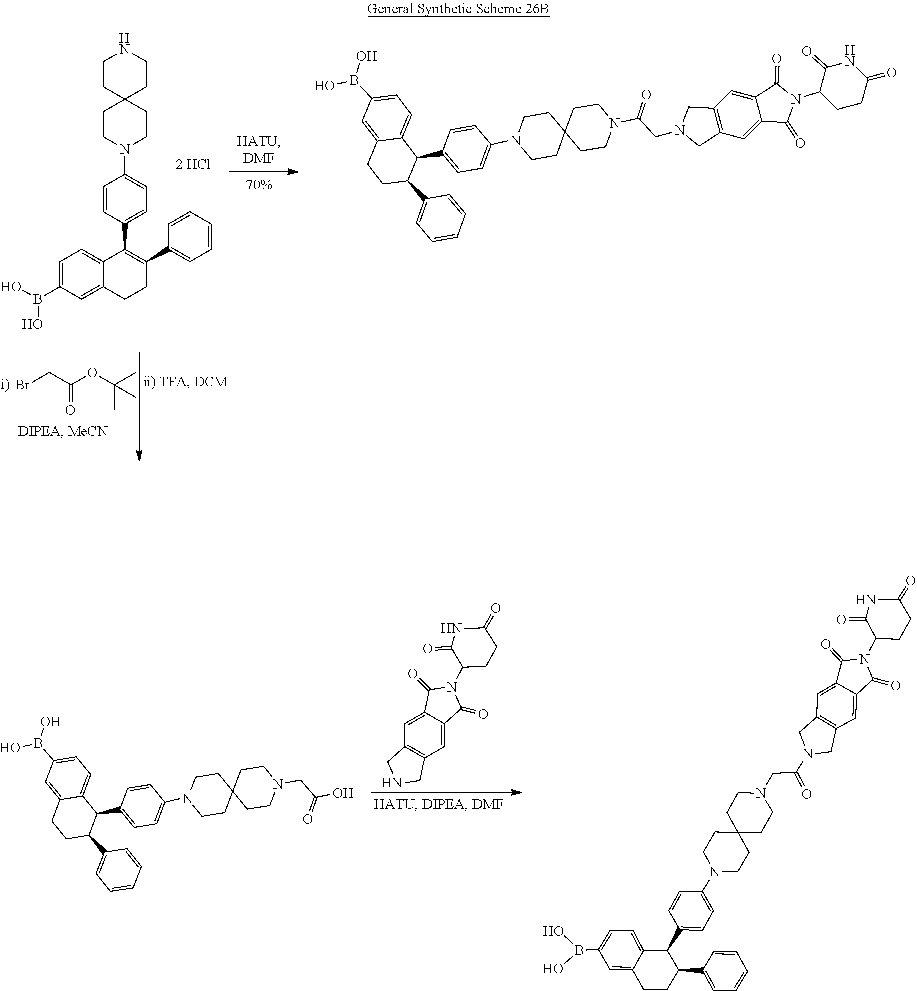

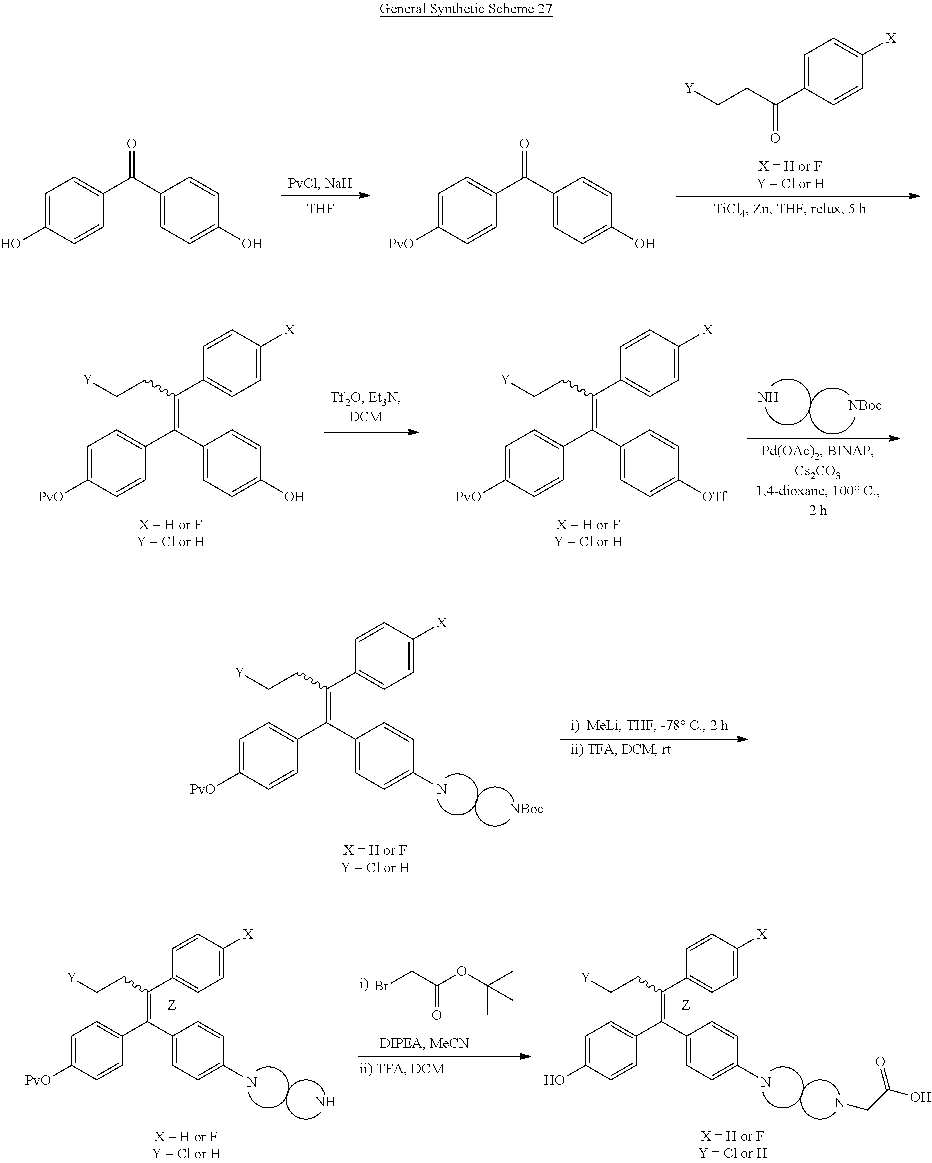

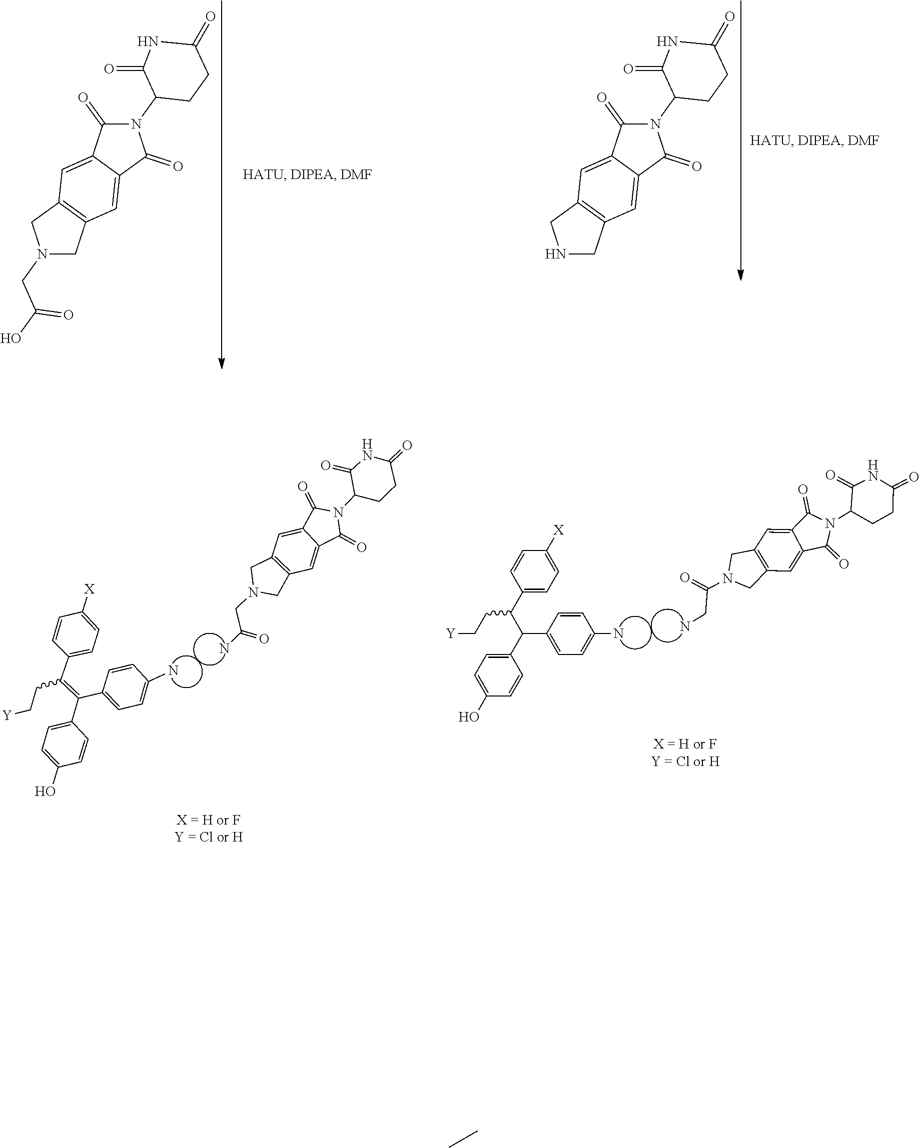

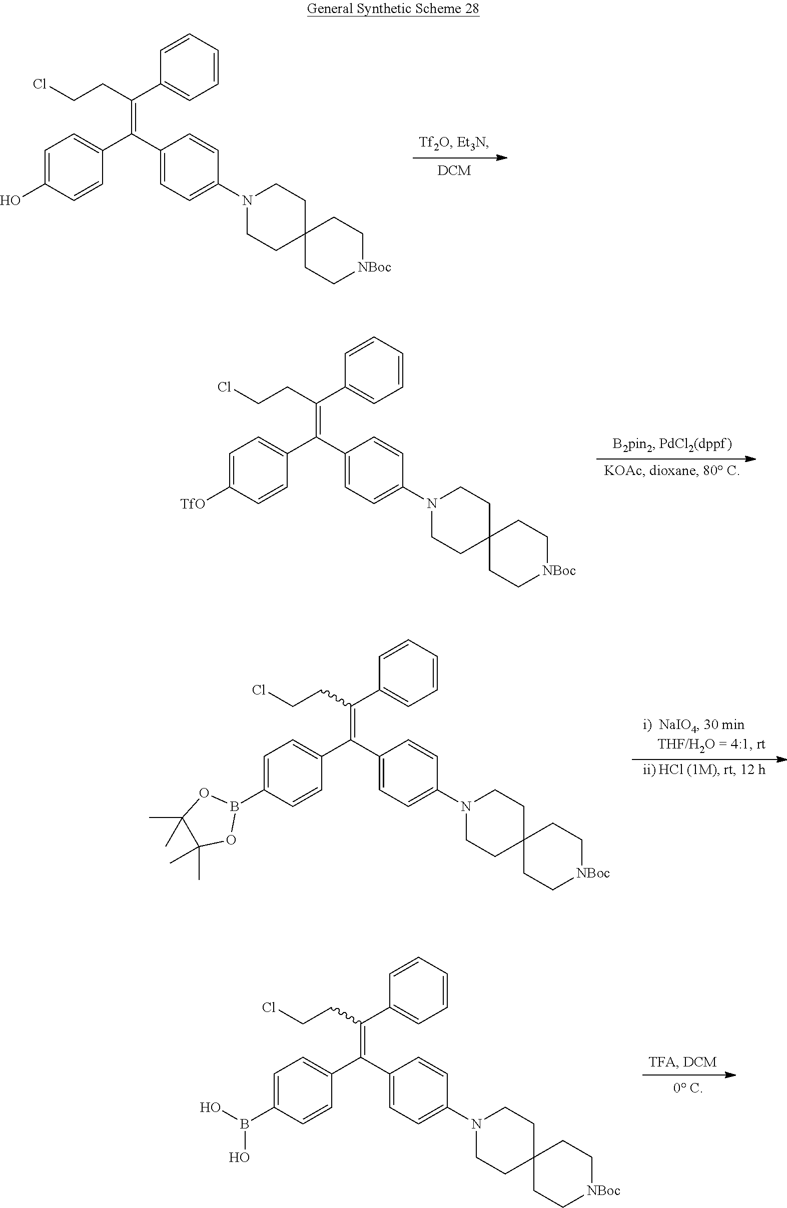 [SMILES: CC(C)(C)OC(=O)N1CCC2(CC1)CCN(c1ccc(/C(=C(/CCCl)c3ccccc3)c3ccc(O)cc3)cc1)CC2 ; CC(C)(C)OC(=O)N1CCC2(CC1)CCN(c1ccc(/C(=C(/CCCl)c3ccccc3)c3ccc(OS(=O)(=O)C(F)(F)F)cc3)cc1)CC2 ; CC(C)(C)OC(=O)N1CCC2(CC1)CCN(c1ccc(C(=C(CCCl)c3ccccc3)c3ccc(B4OC(C)(C)C(C)(C)O4)cc3)cc1)CC2 ; CC(C)(C)OC(=O)N1CCC2(CC1)CCN(c1ccc(C(=C(CCCl)c3ccccc3)c3ccc(B(O)O)cc3)cc1)CC2]

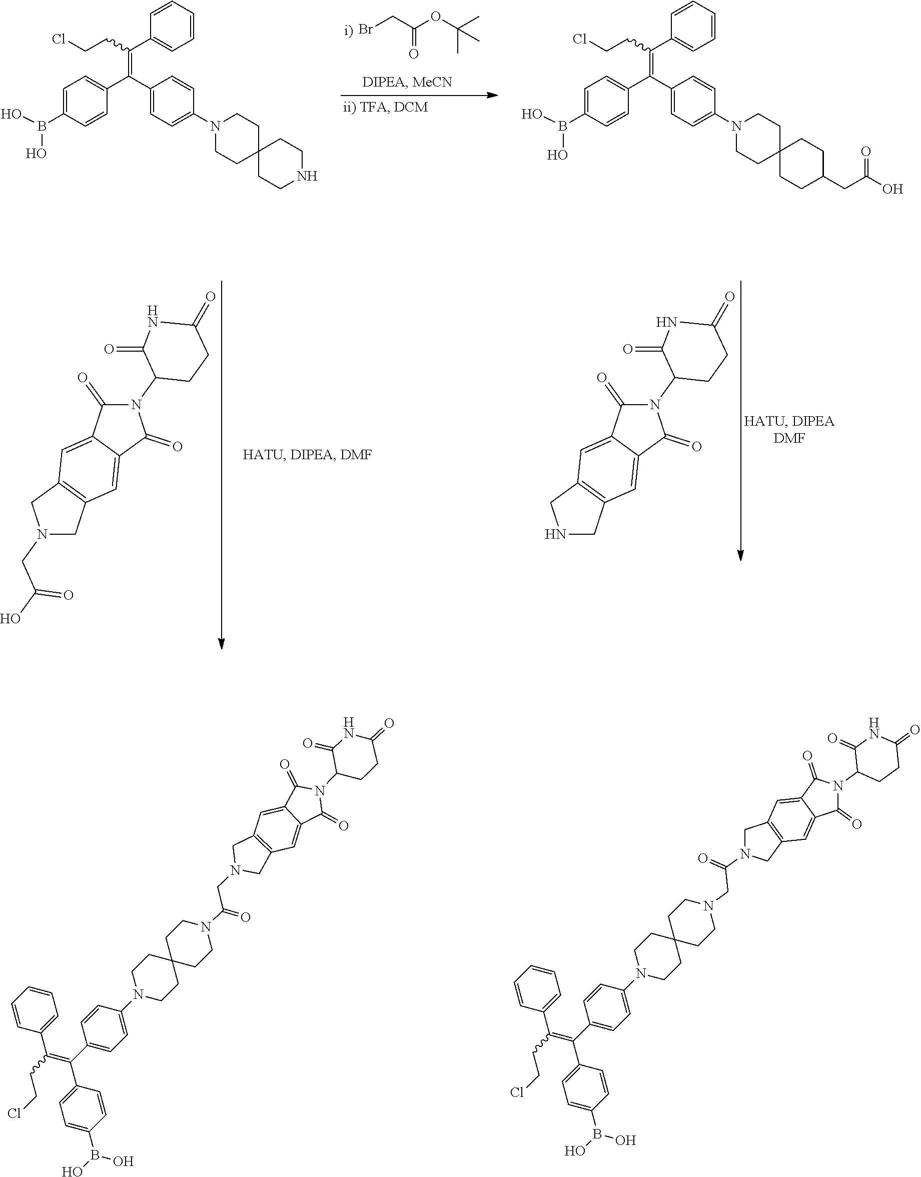

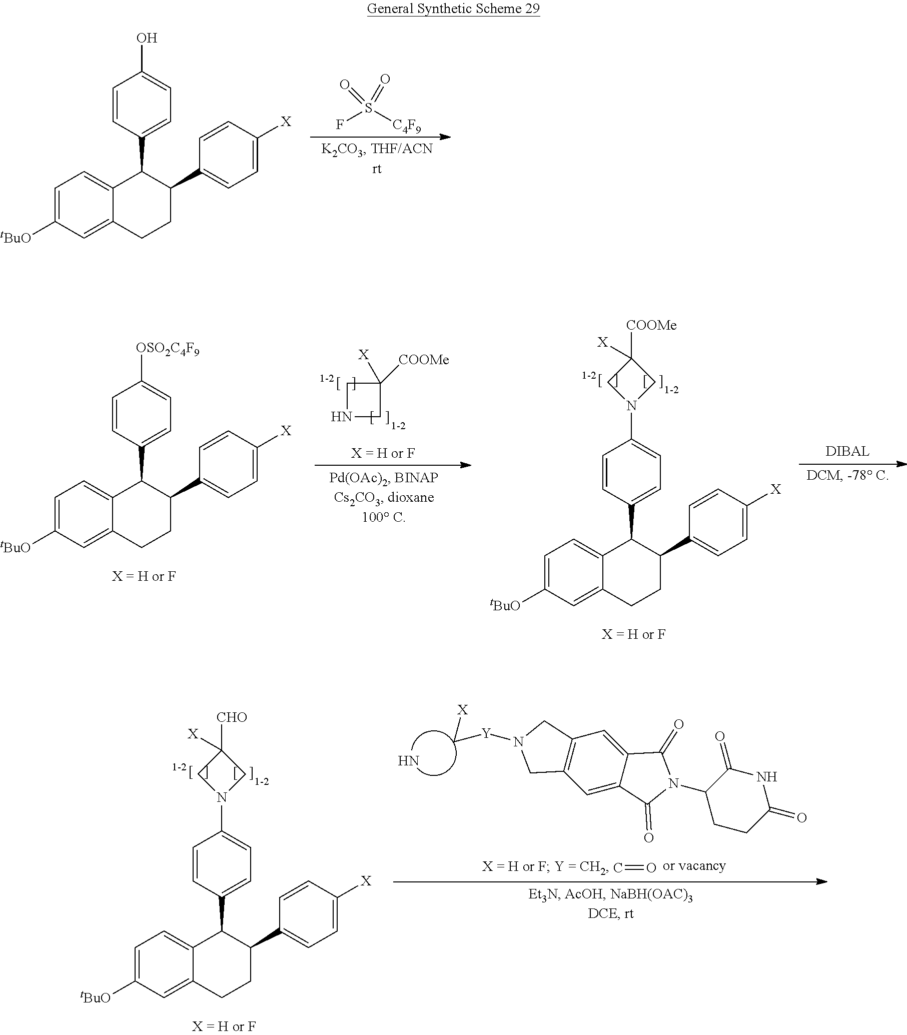

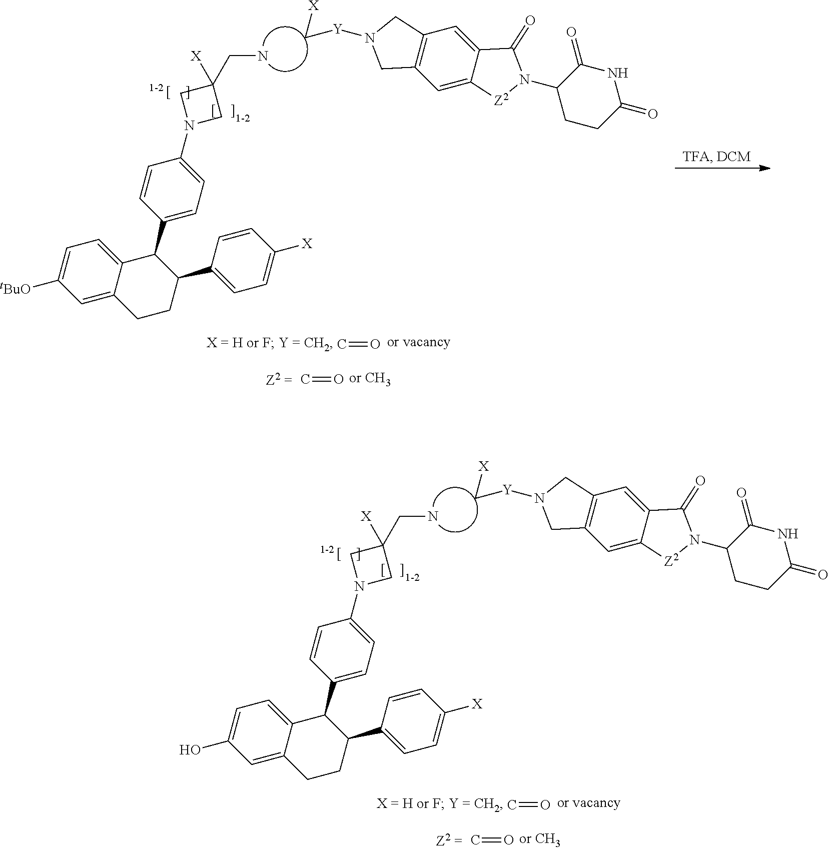

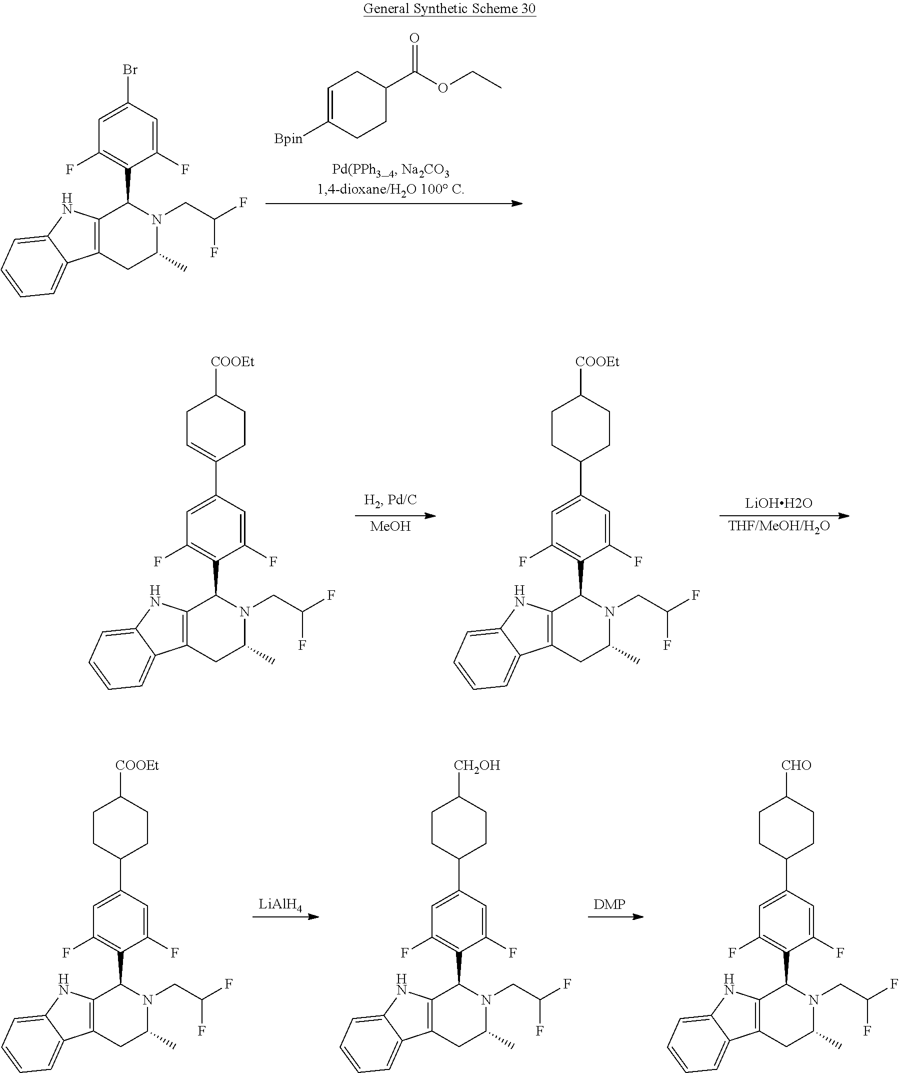

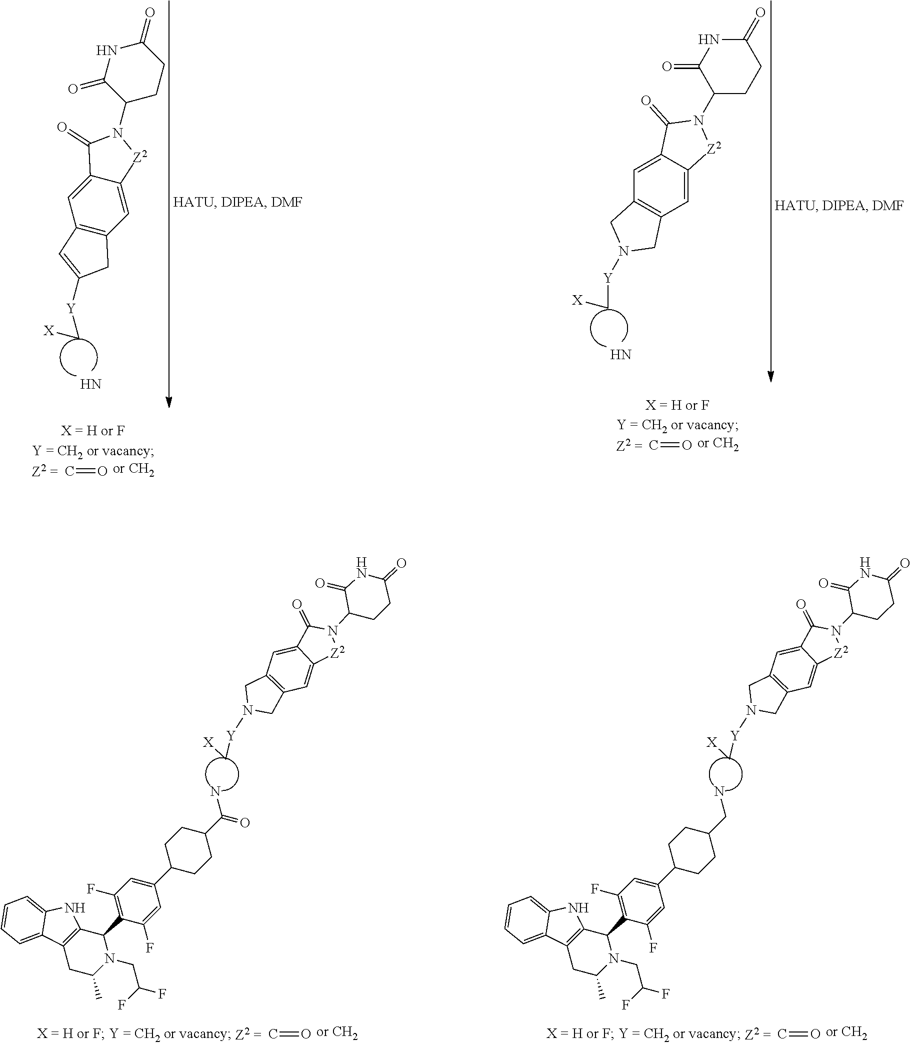

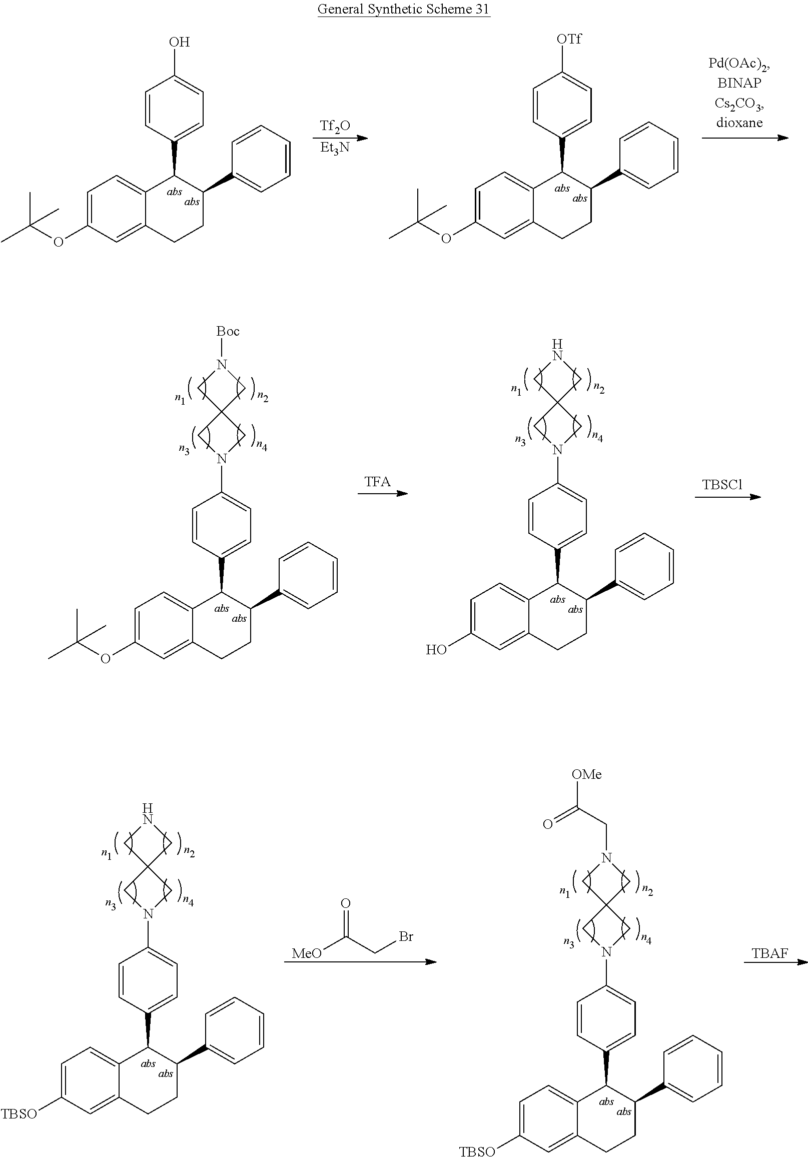

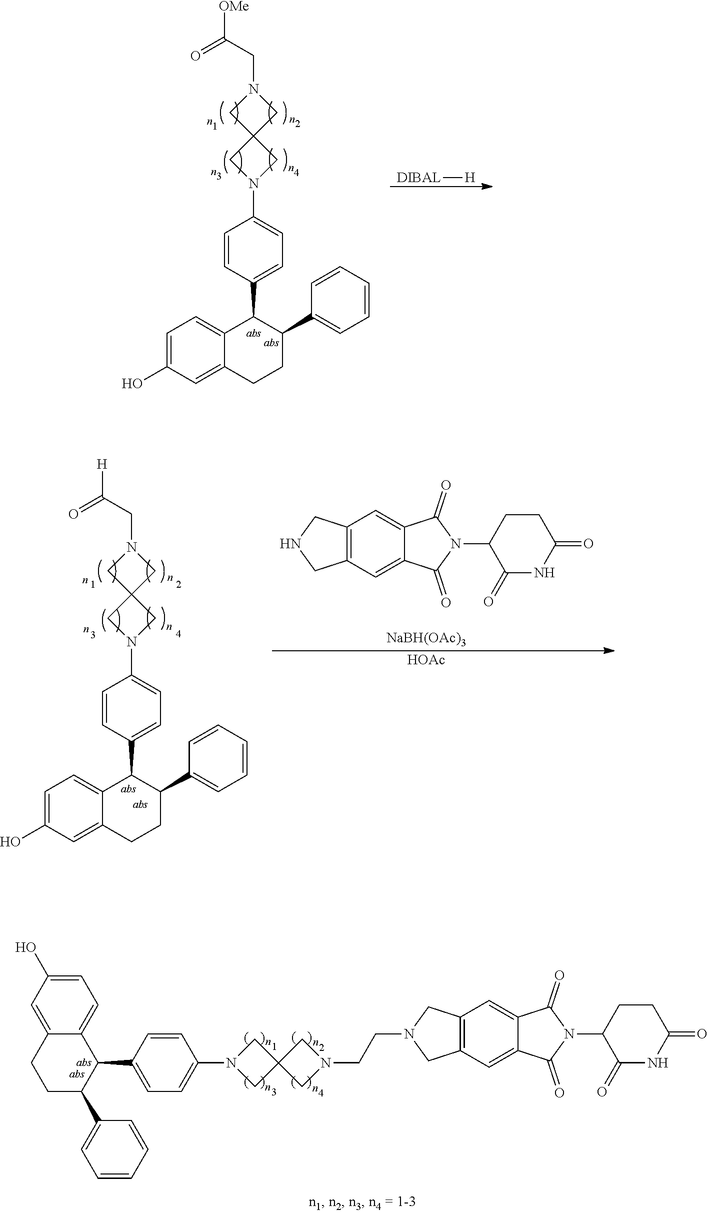

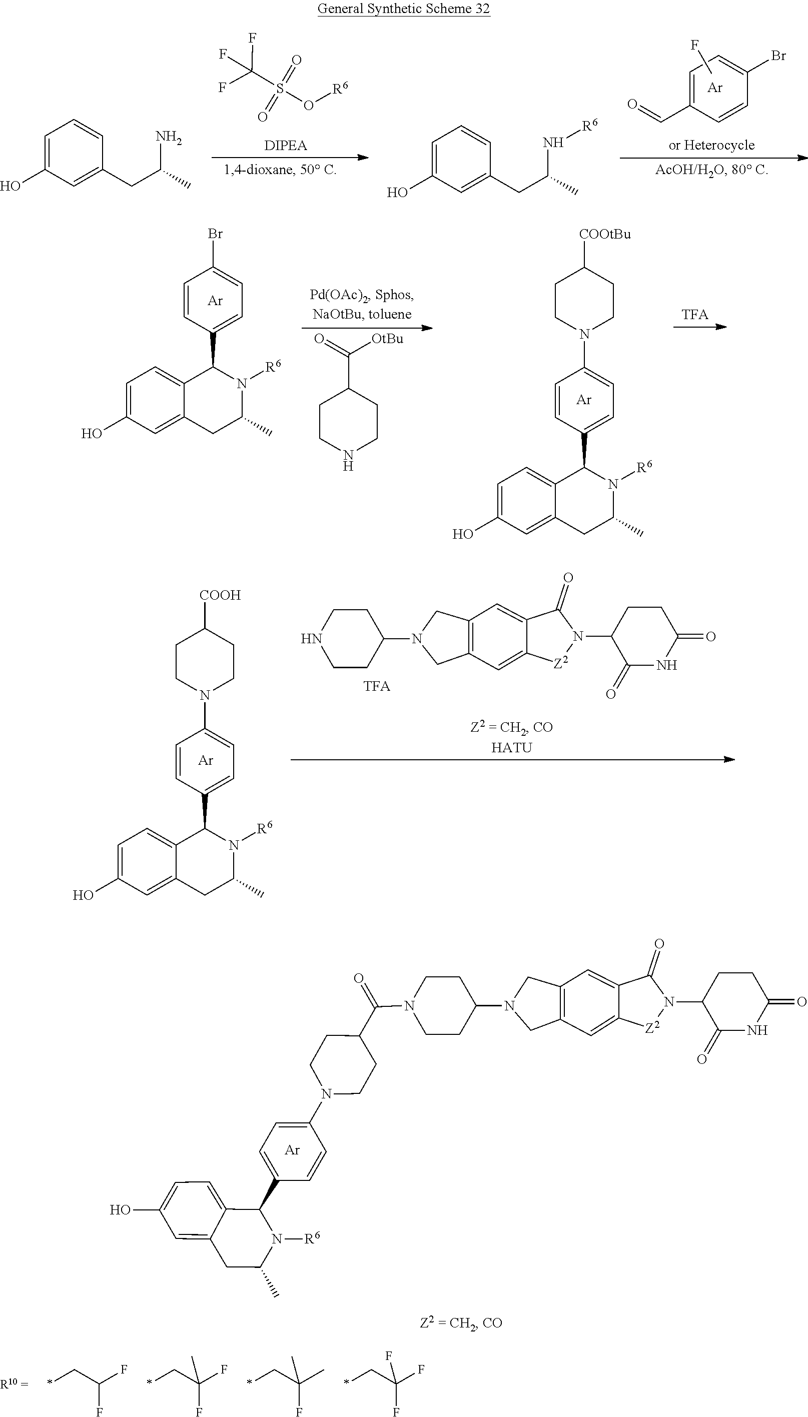

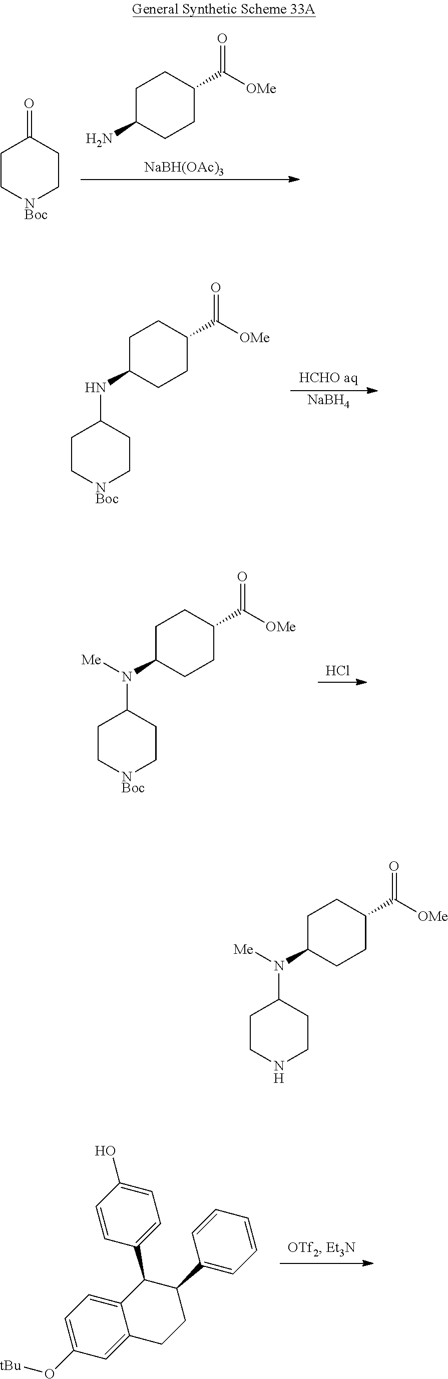

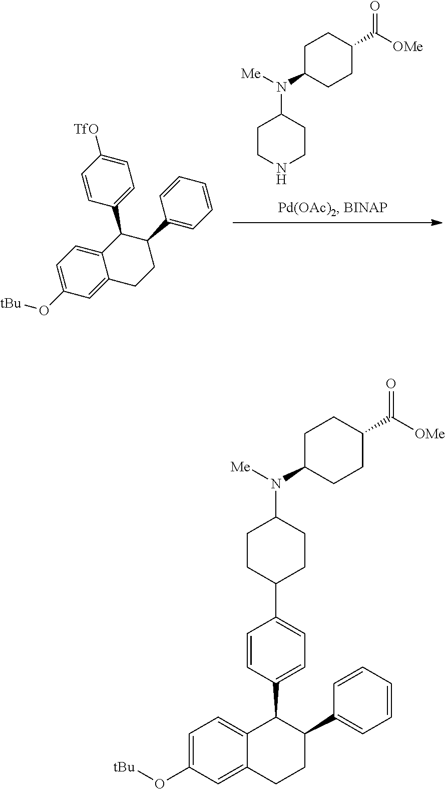 [SMILES: CC(C)(C)Oc1ccc2c(c1)CC[C@H](c1ccccc1)[C@@H]2c1ccc(OS(=O)(=O)C(F)(F)F)cc1 ; COC(=O)[C@H]1CC[C@H](N(C)C2CCNCC2)CC1 ; COC(=O)[C@H]1CC[C@H](N(C)C2CCC(c3ccc([C@@H]4c5ccc(OC(C)(C)C)cc5CC[C@@H]4c4ccccc4)cc3)CC2)CC1]

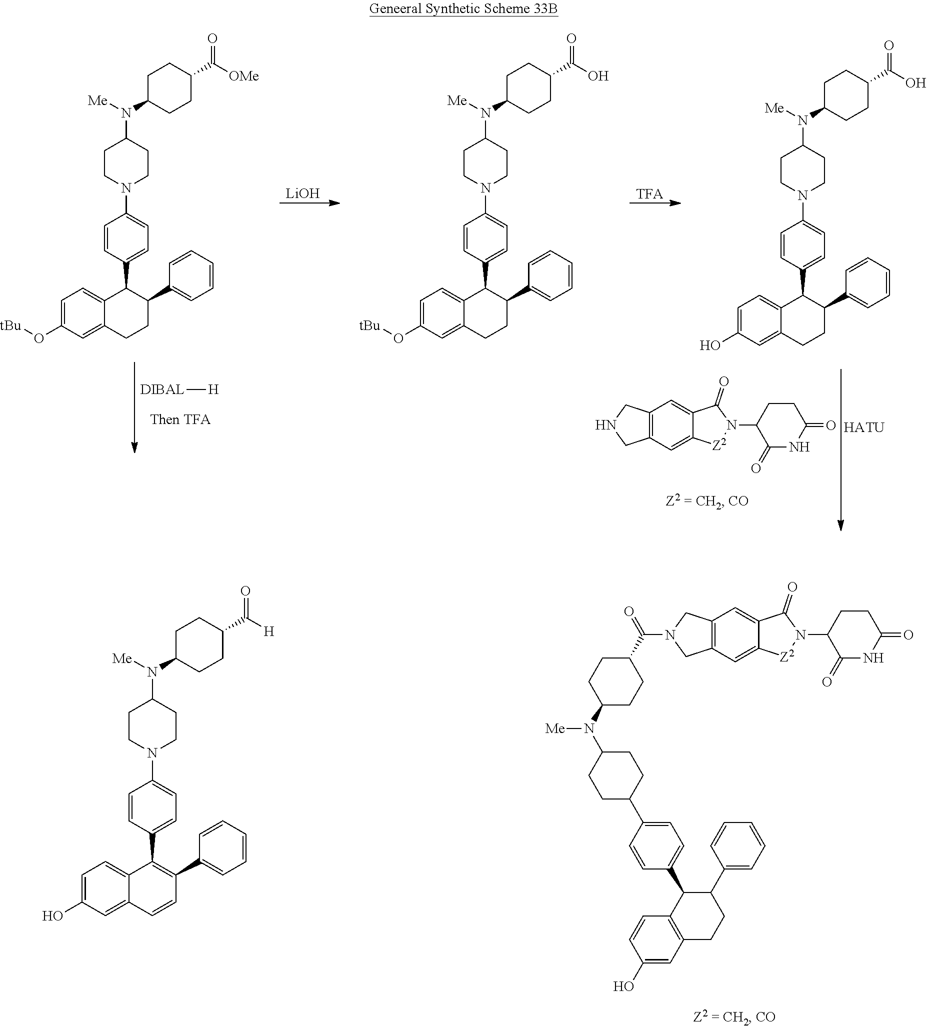

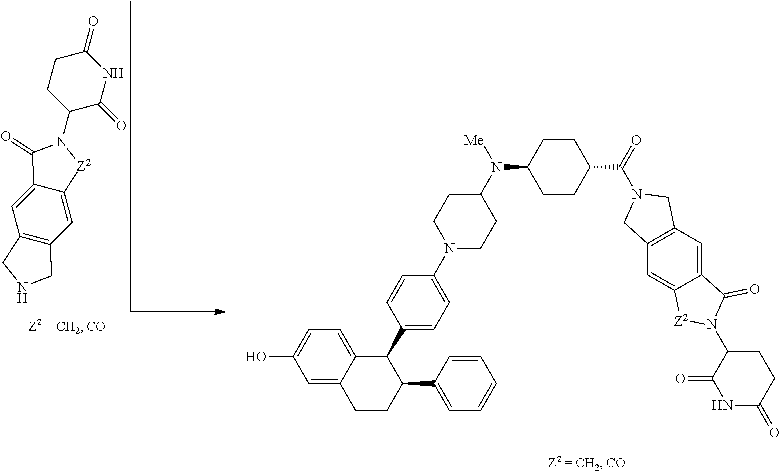

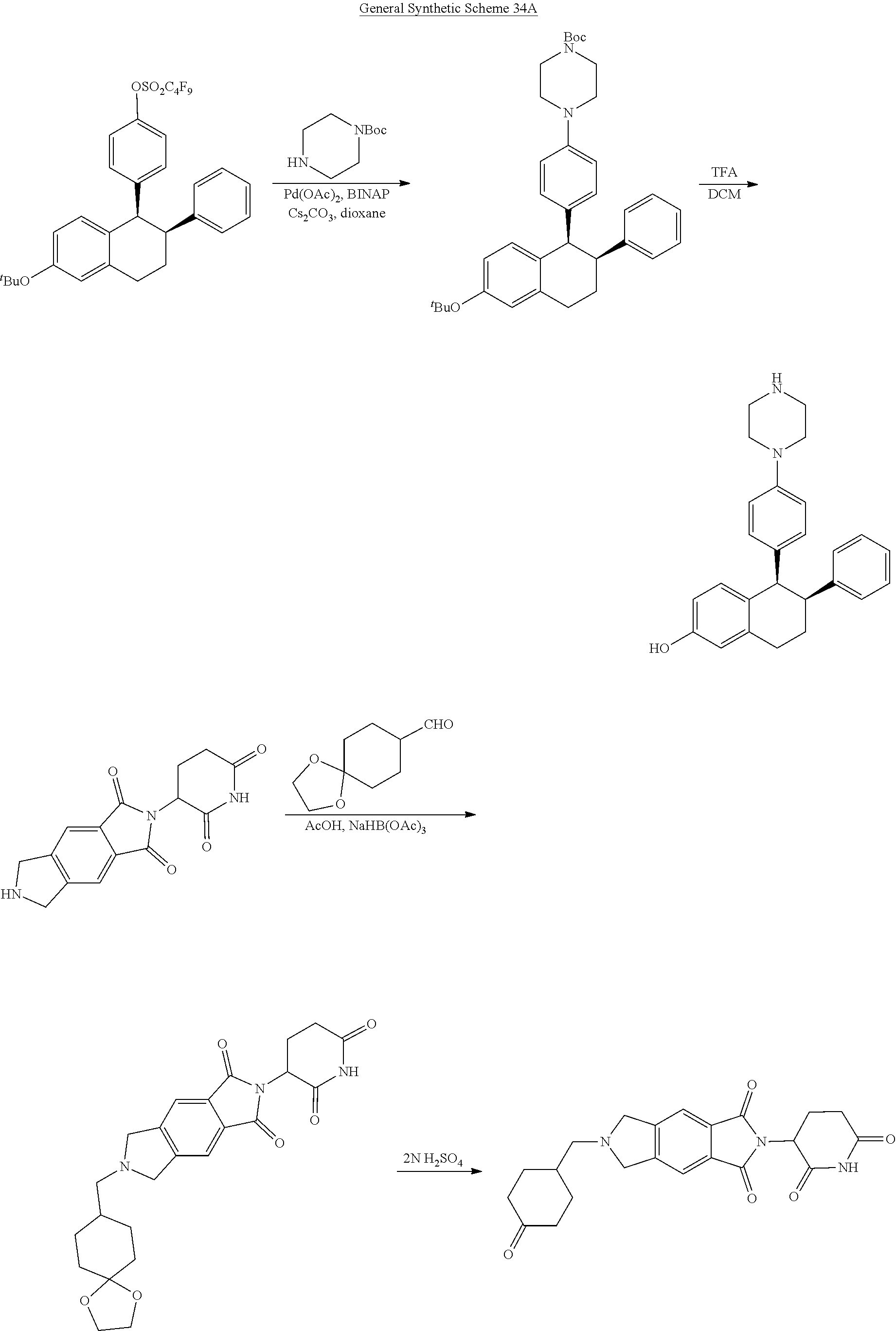

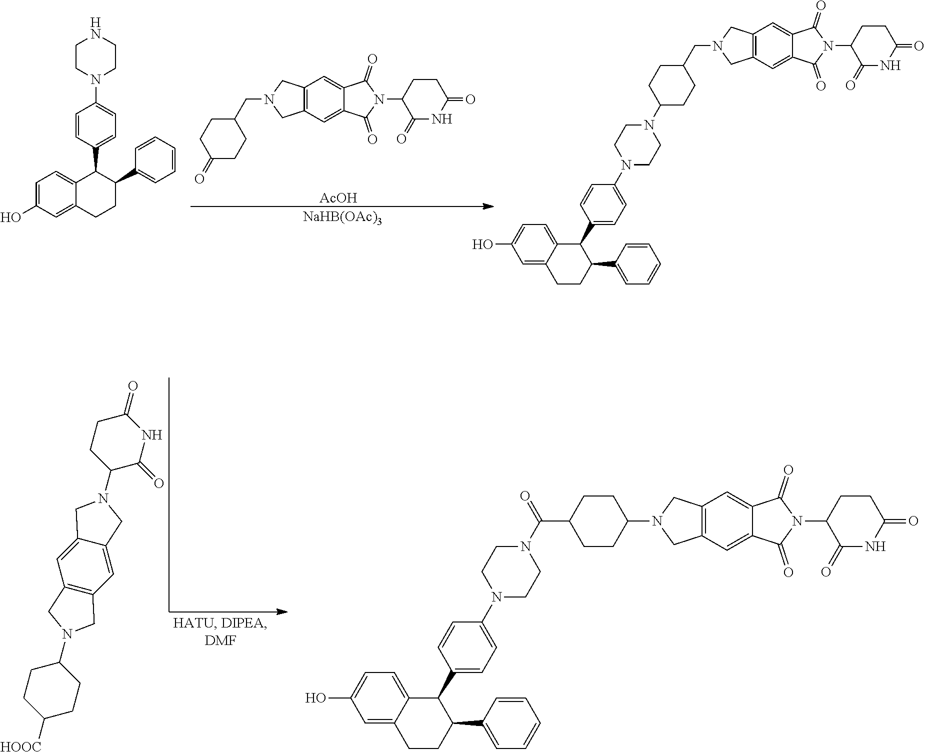

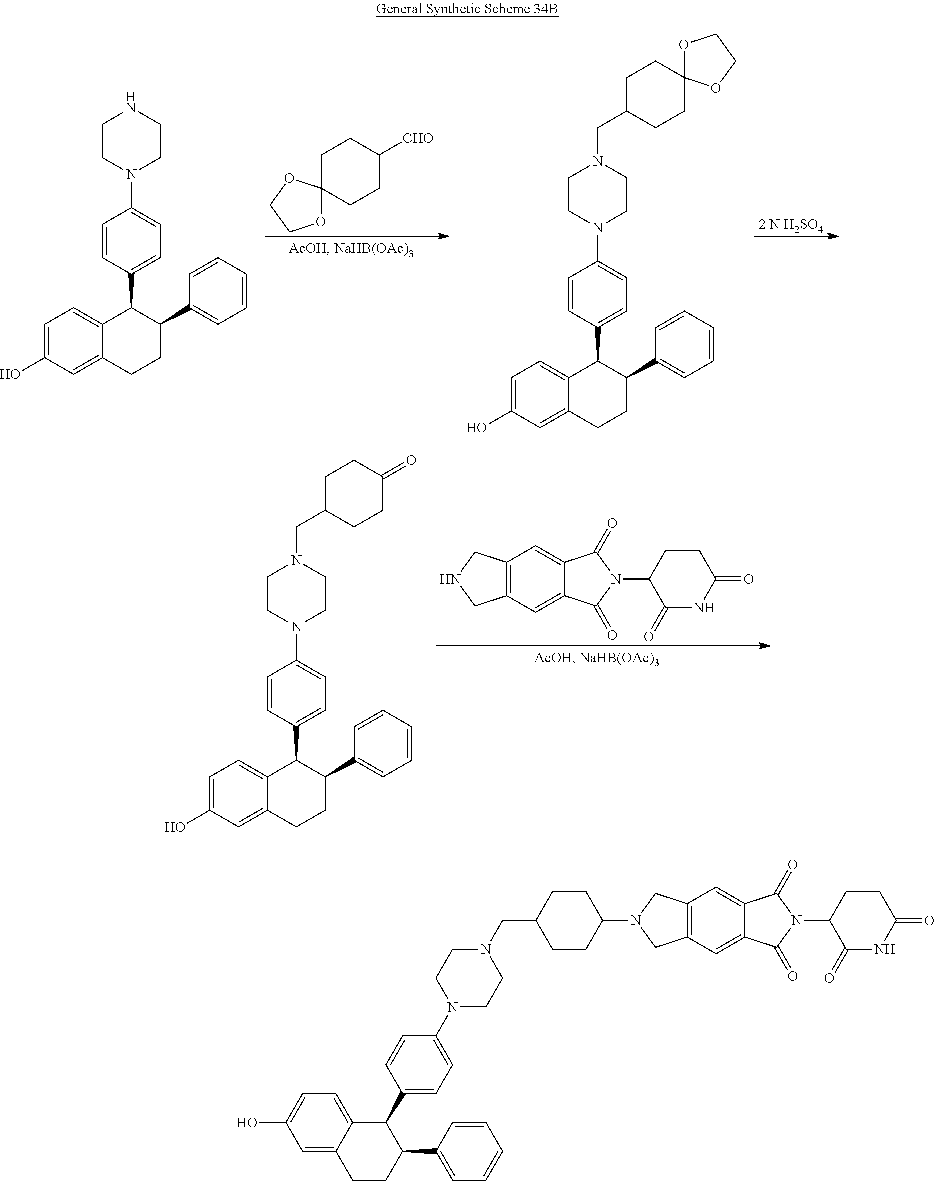

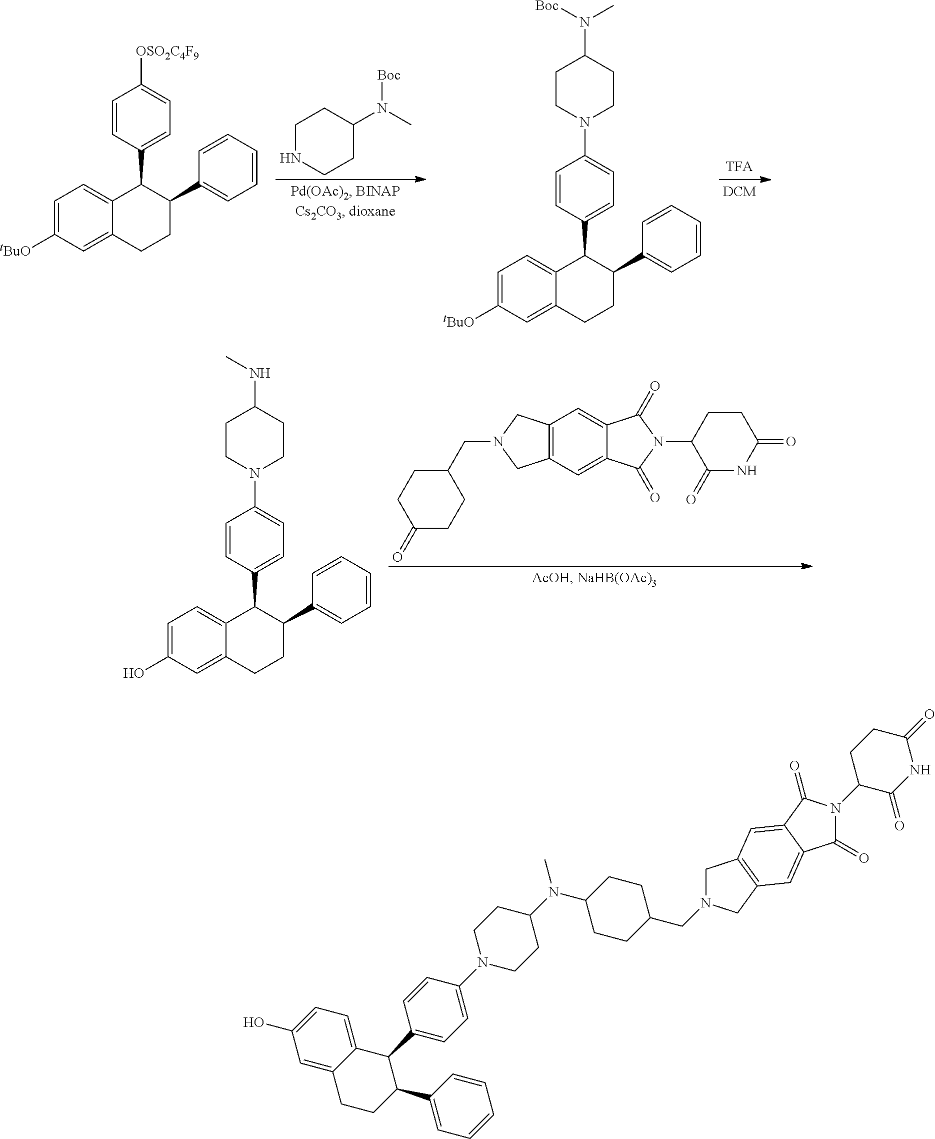

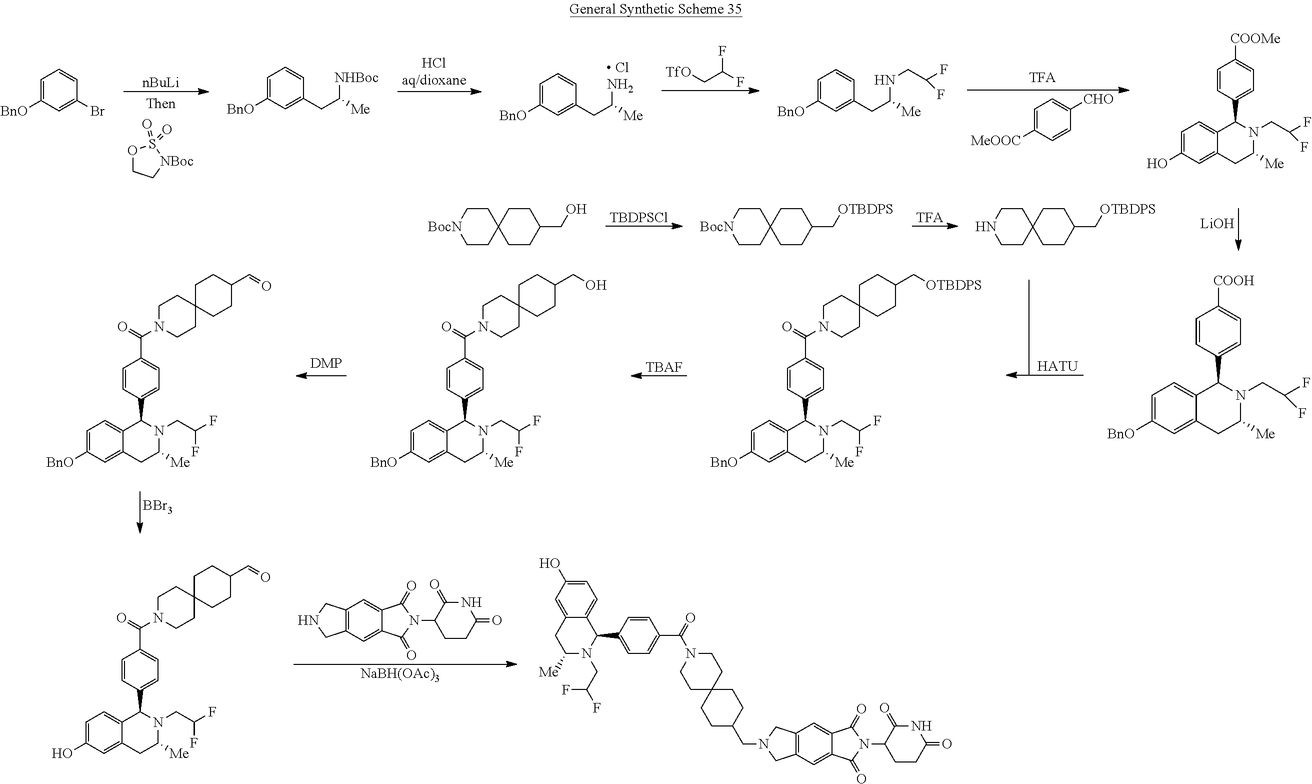

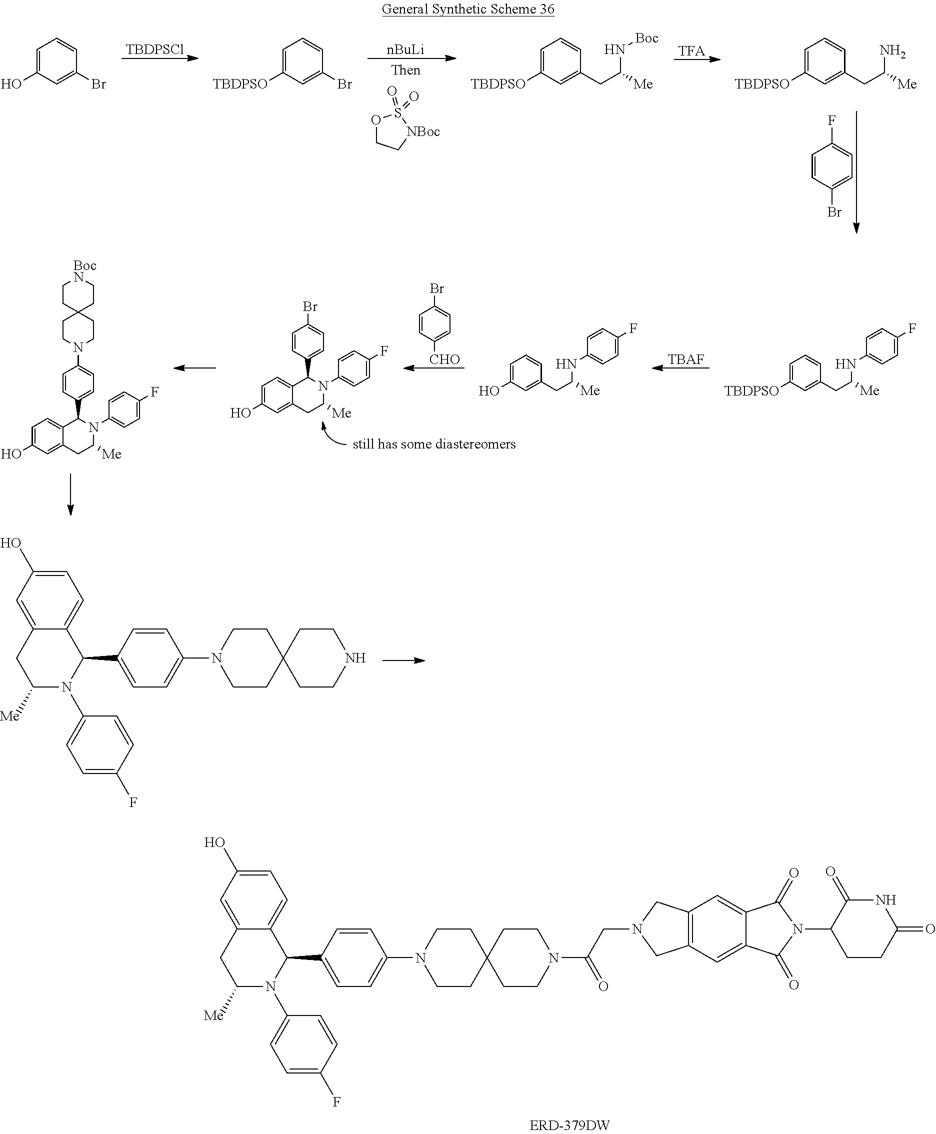

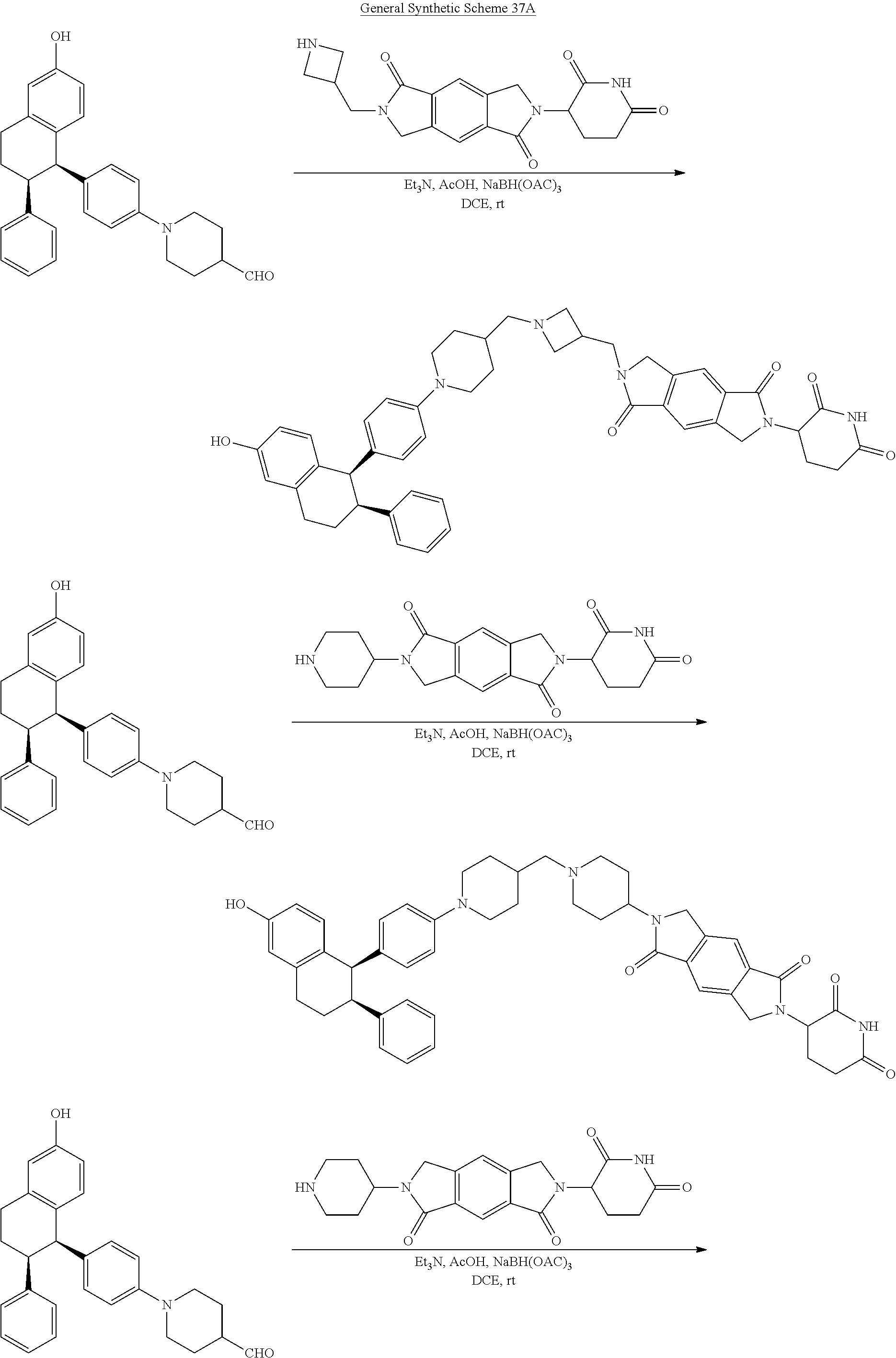

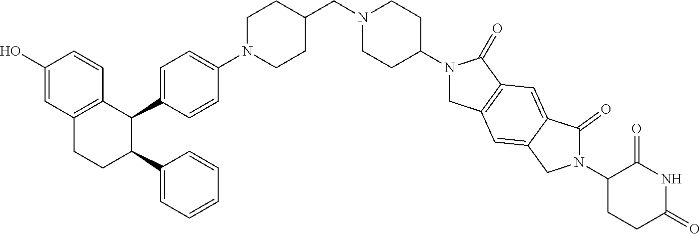 [SMILES: O=C1CCC(N2Cc3cc4c(cc3C2=O)C(=O)N(C2CCN(CC3CCN(c5ccc([C@@H]6c7ccc(O)cc7CC[C@@H]6c6ccccc6)cc5)CC3)CC2)C4)C(=O)N1]

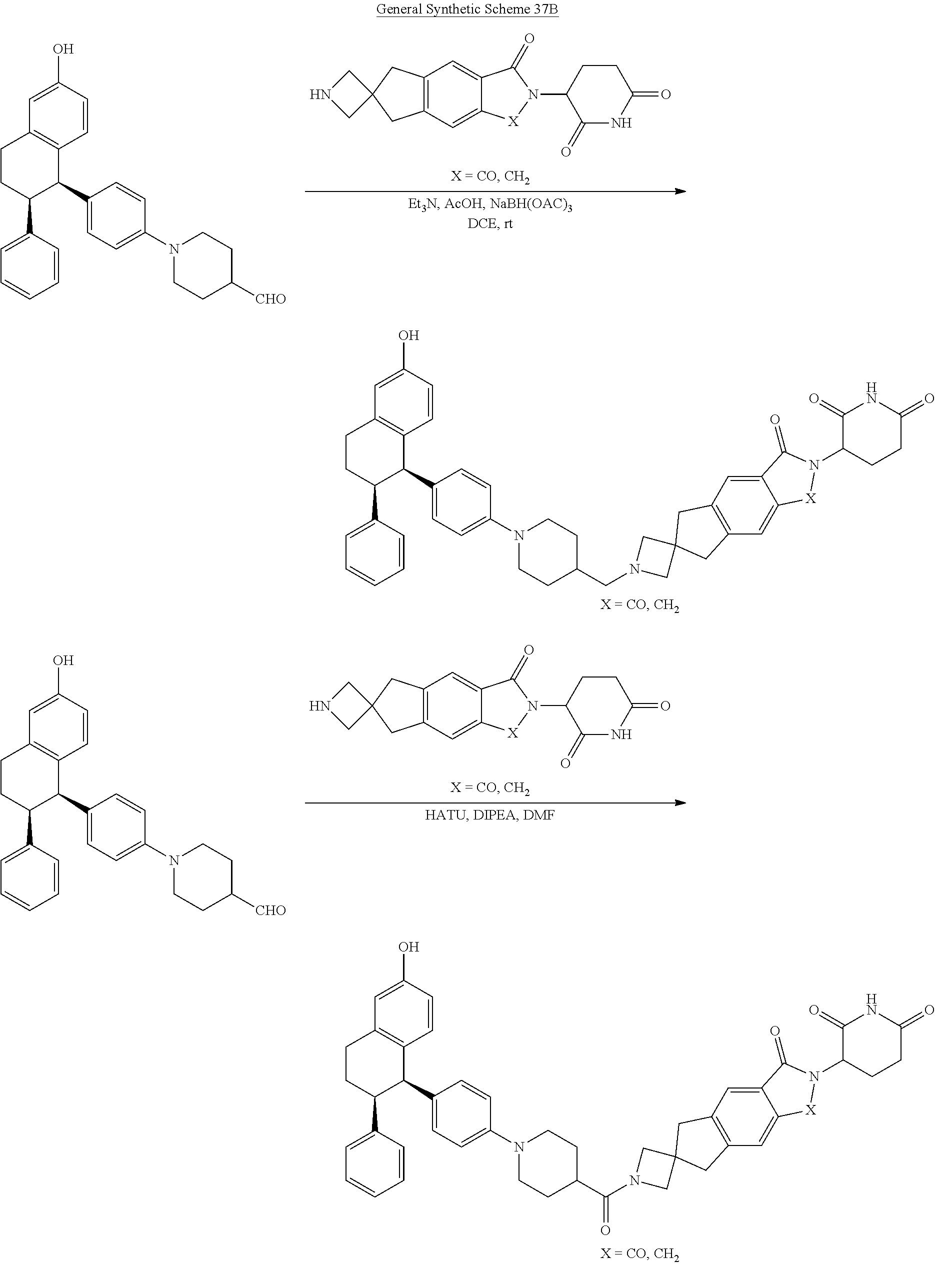

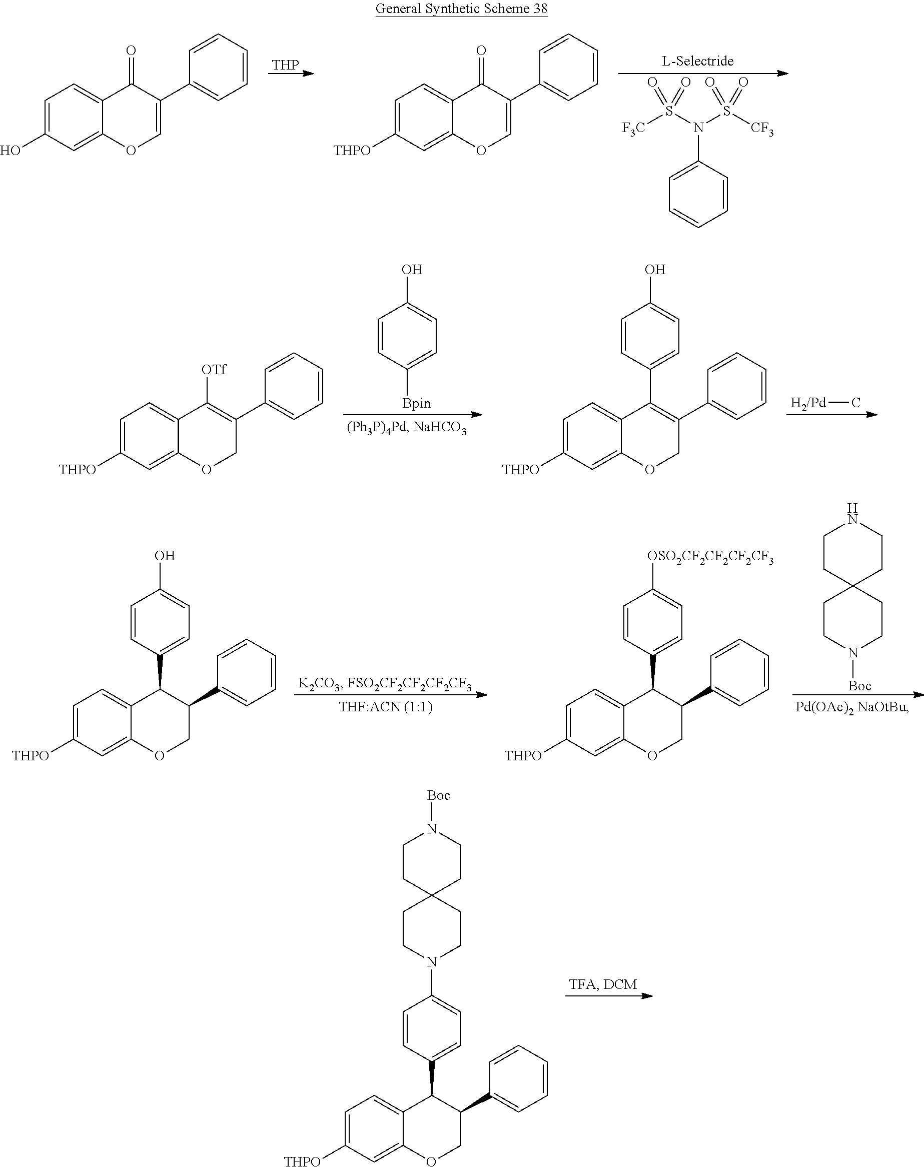

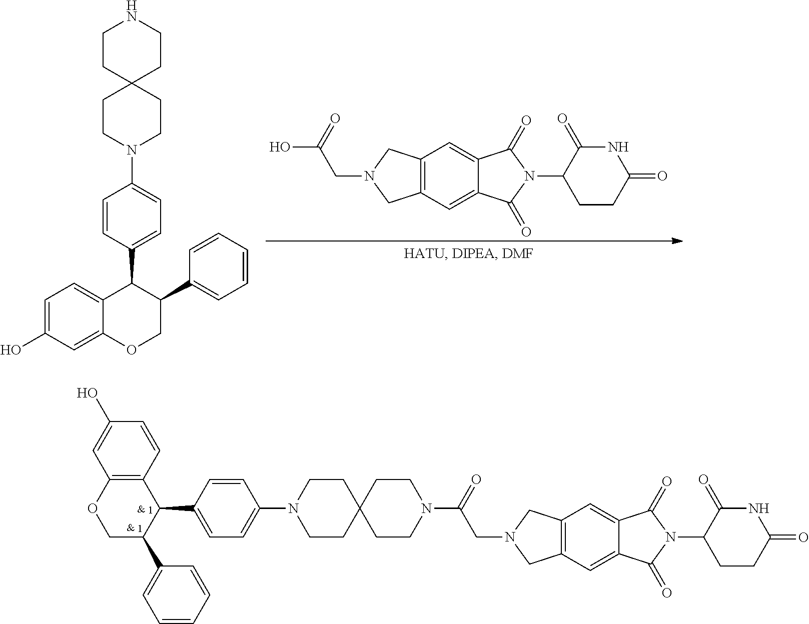 [SMILES: Oc1ccc2c(c1)OC[C@H](c1ccccc1)[C@@H]2c1ccc(N2CCC3(CCNCC3)CC2)cc1 ; O=C(O)CN1Cc2cc3c(cc2C1)C(=O)N(C1CCC(=O)NC1=O)C3=O ; O=C1CCC(N2C(=O)c3cc4c(cc3C2=O)CN(CC(=O)N2CCC3(CC2)CCN(c2ccc([C@@H]5c6ccc(O)cc6OC[C@@H]5c5ccccc5)cc2)CC3)C4)C(=O)N1]

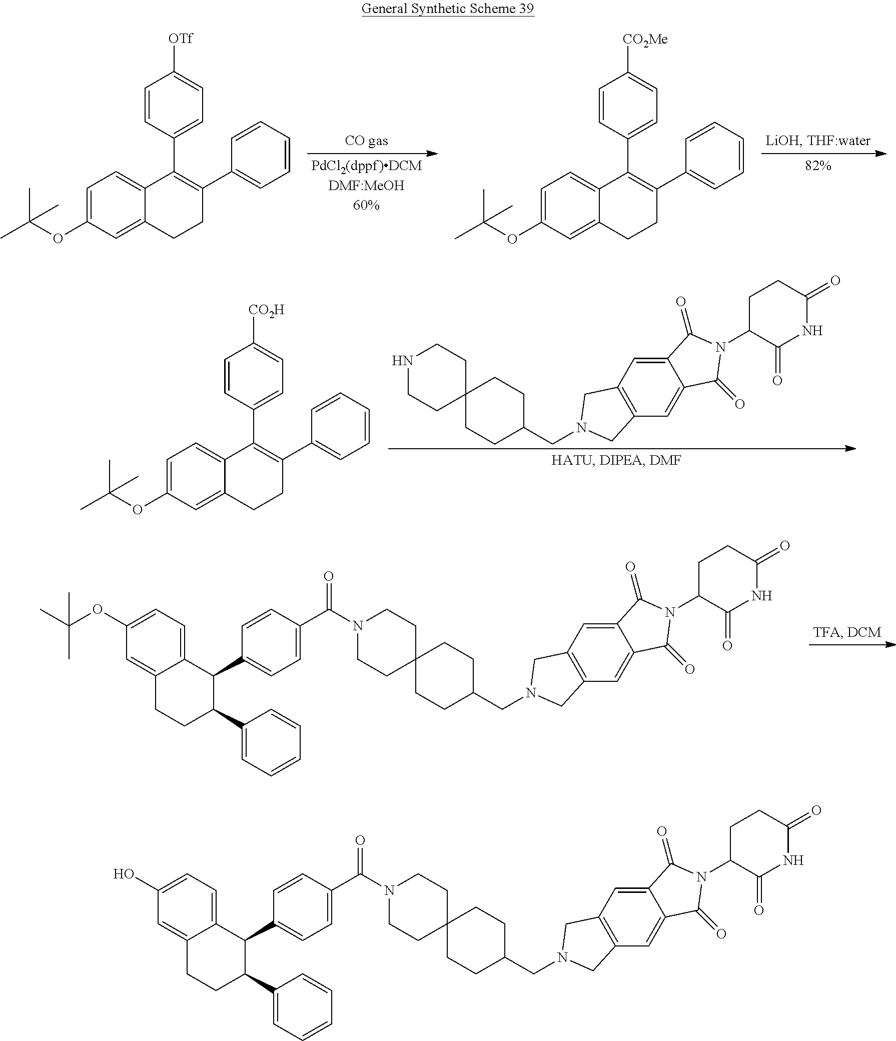

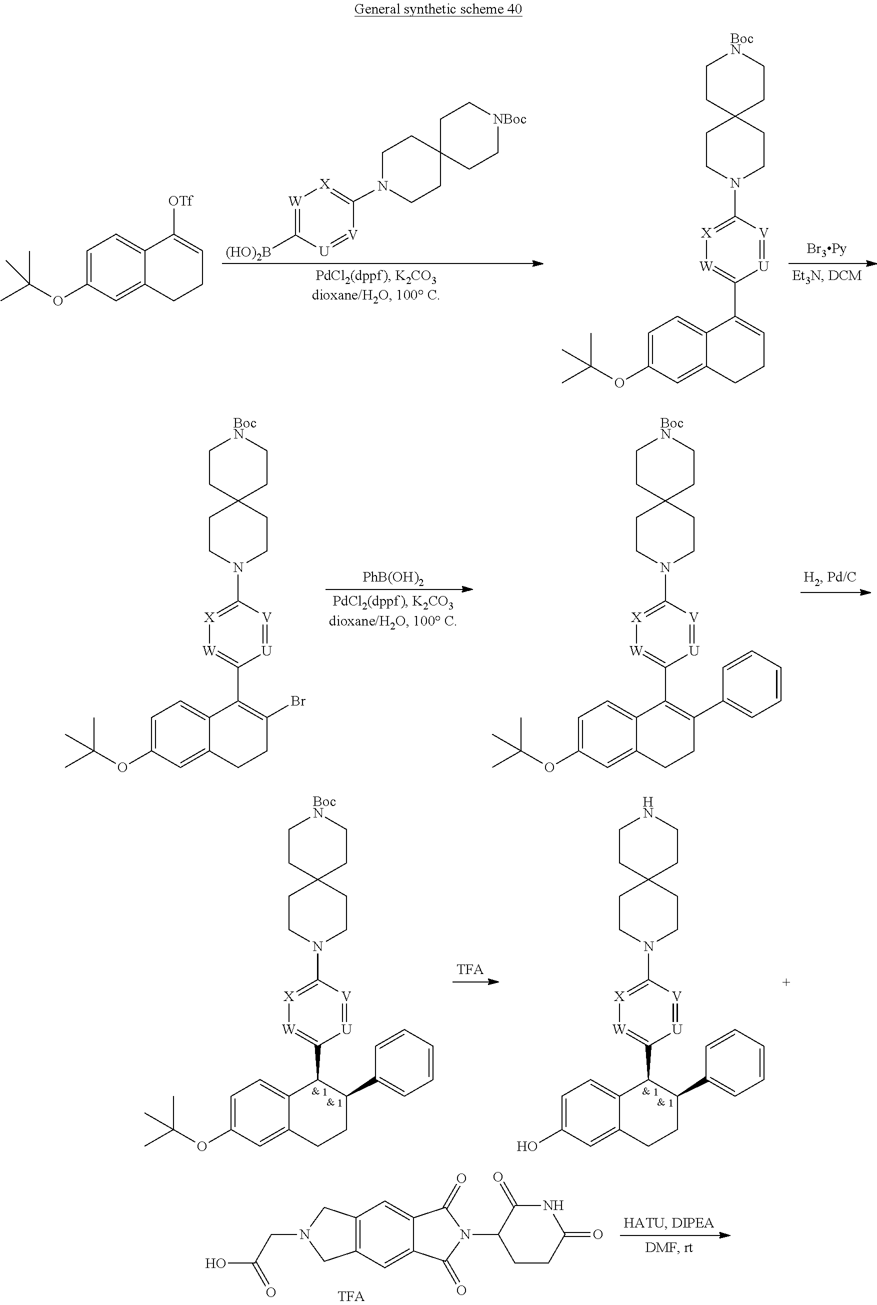

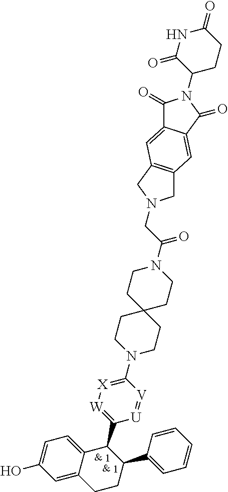

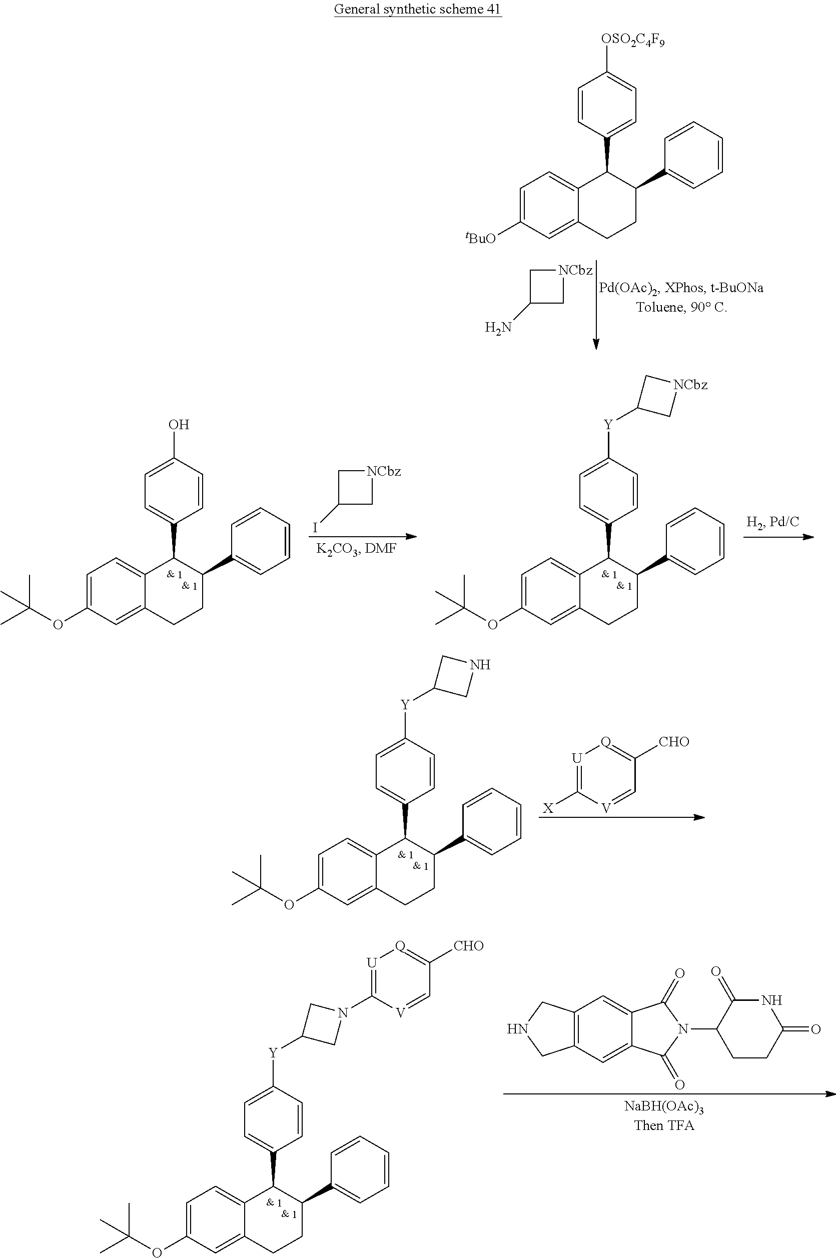

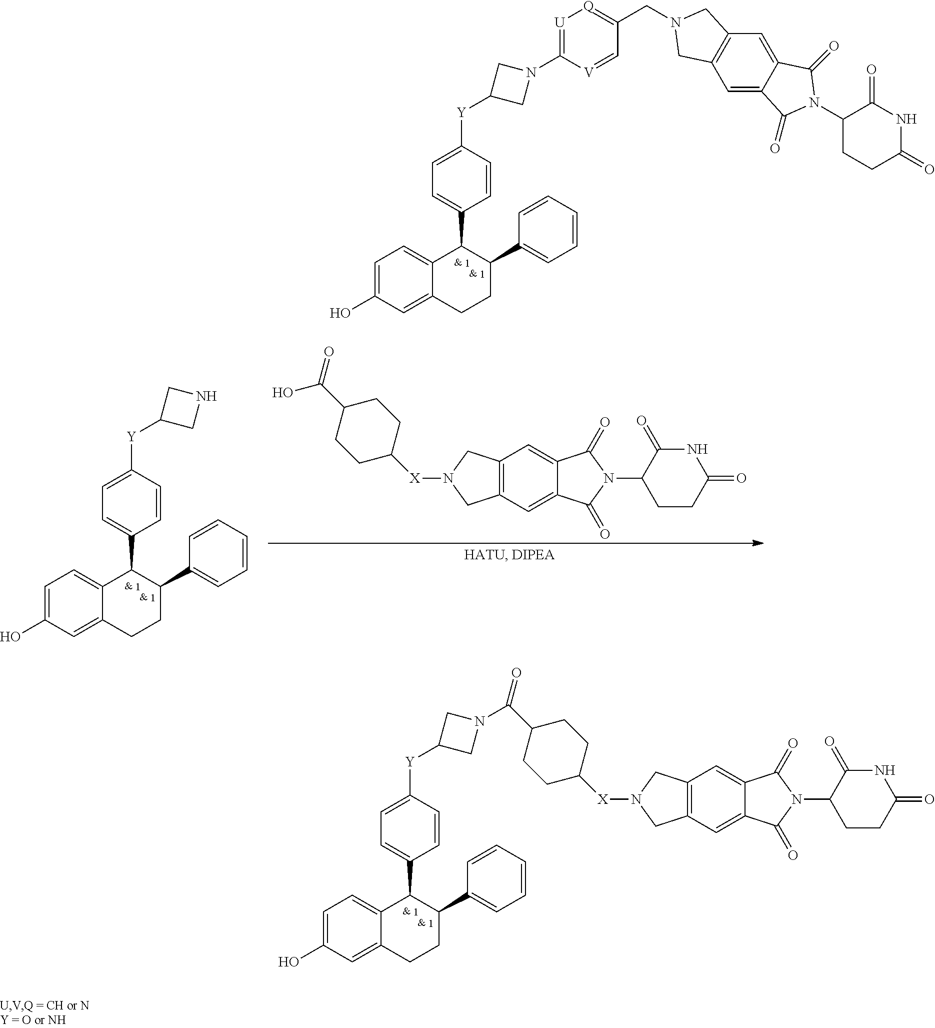

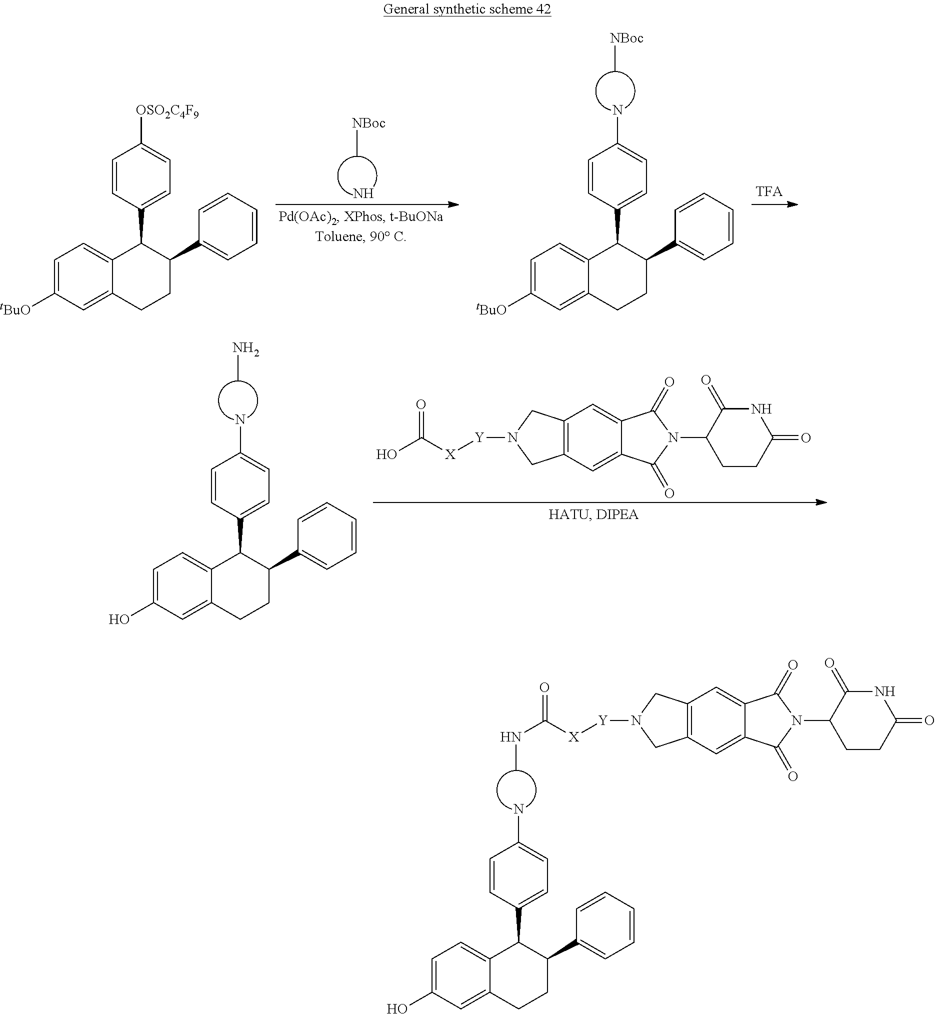

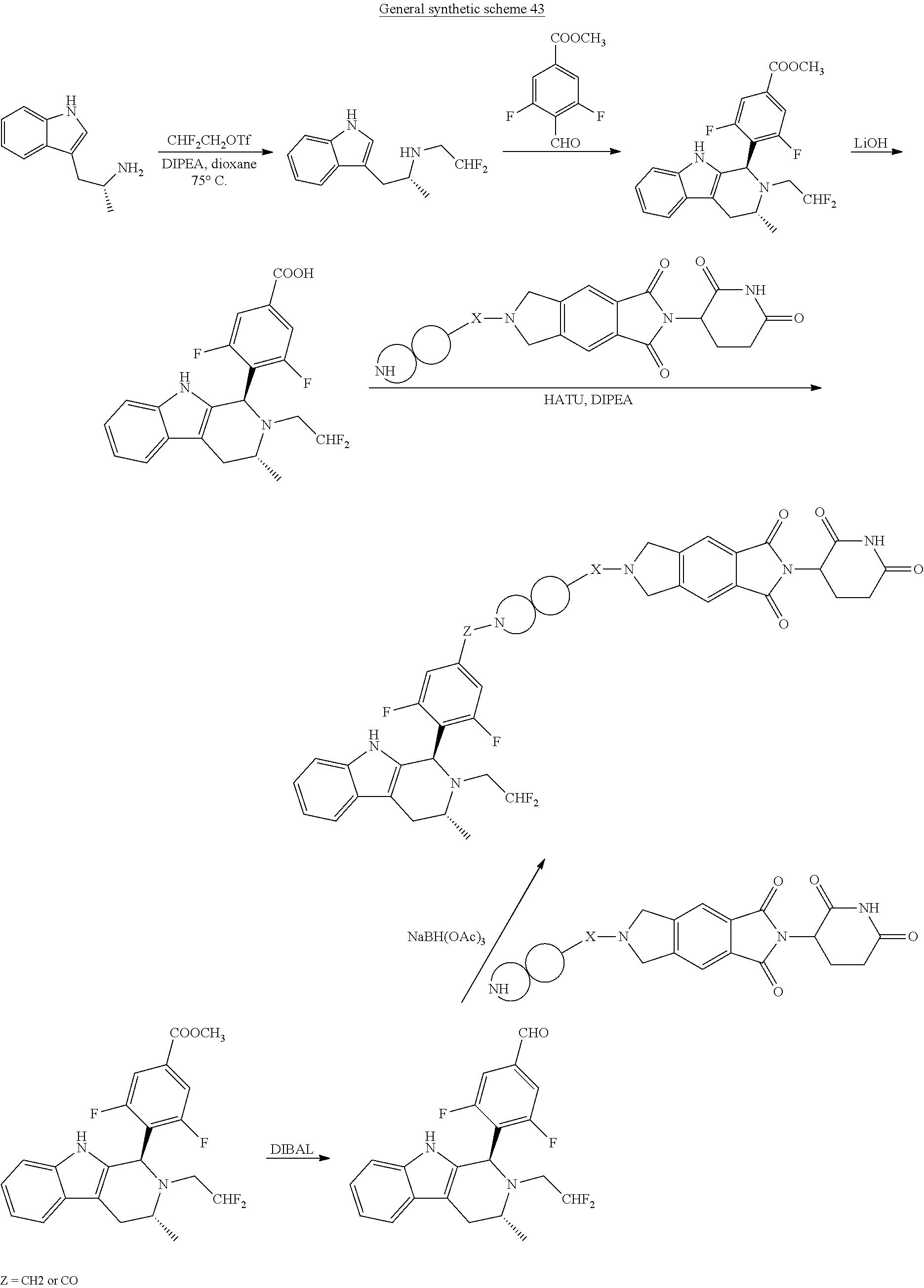

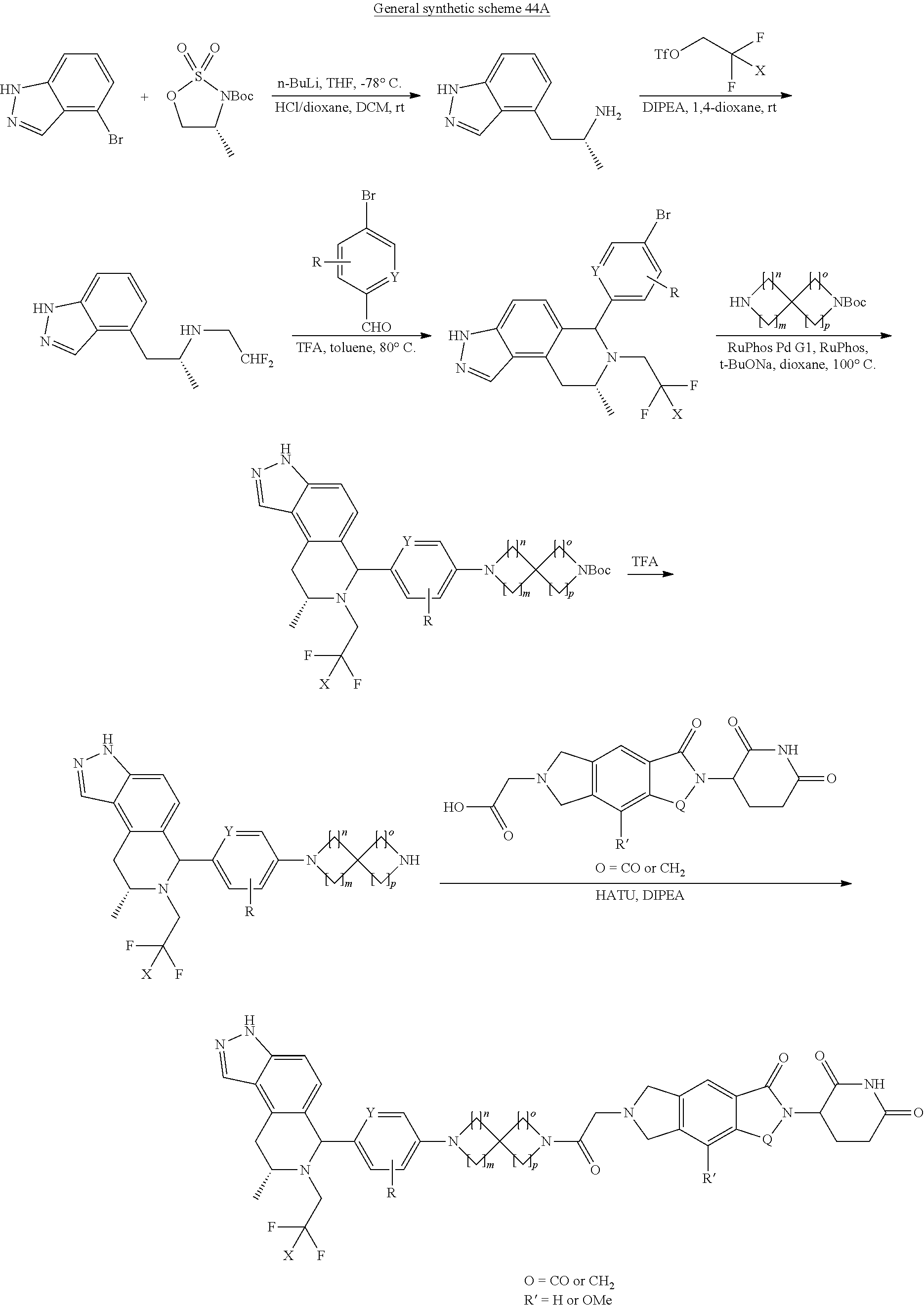

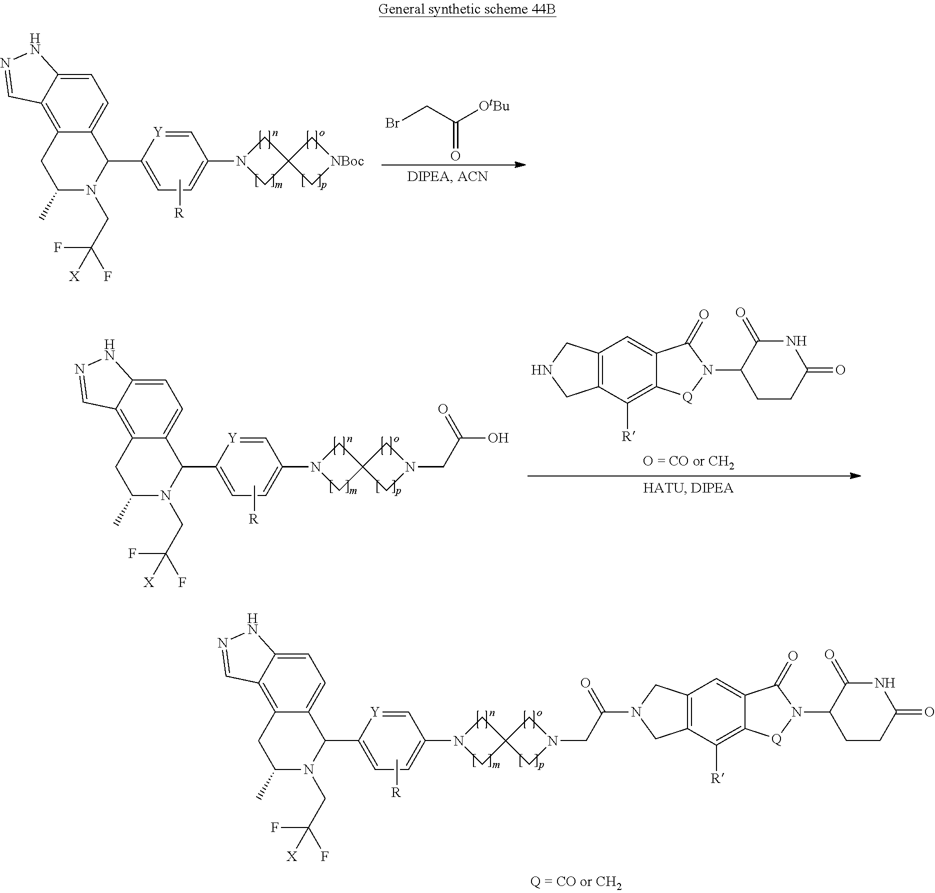

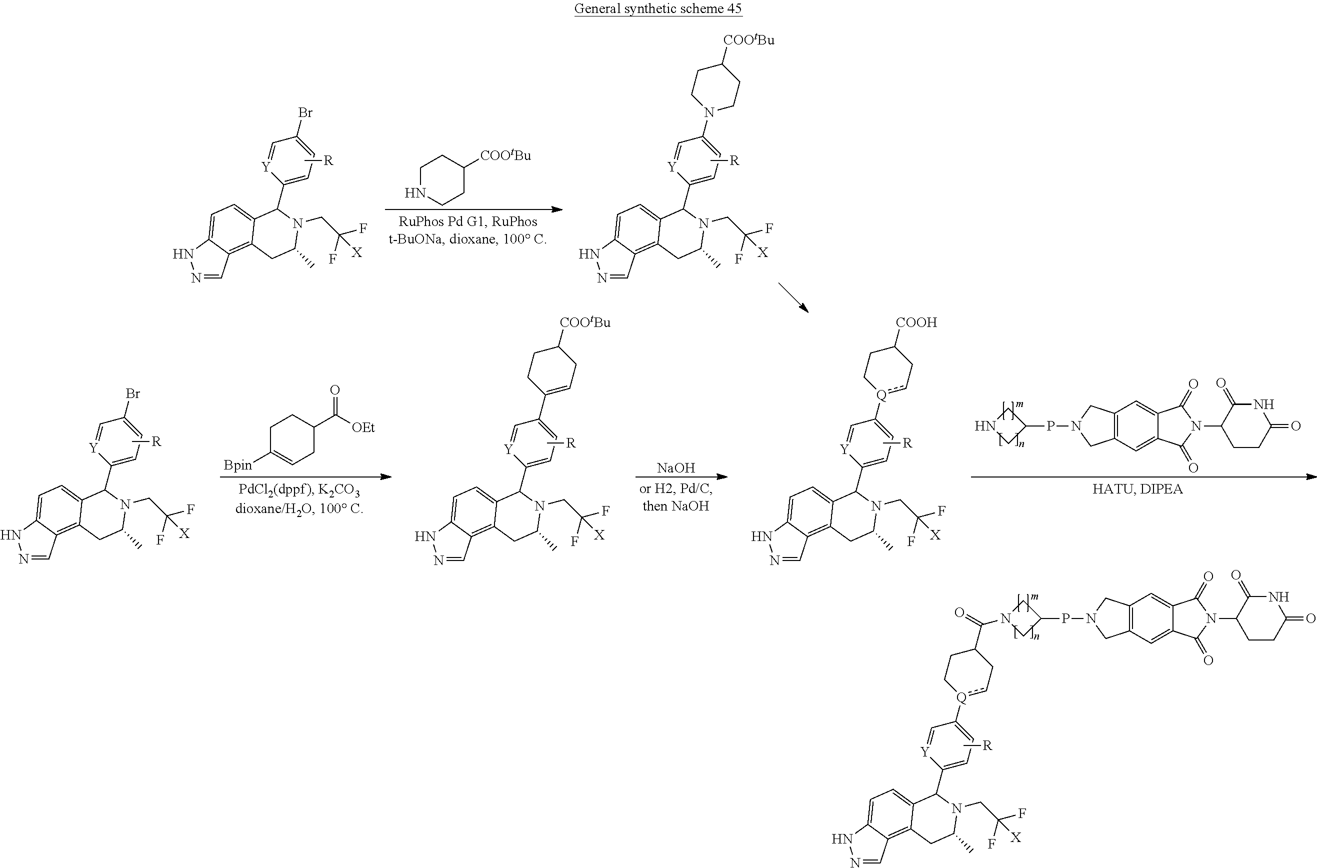

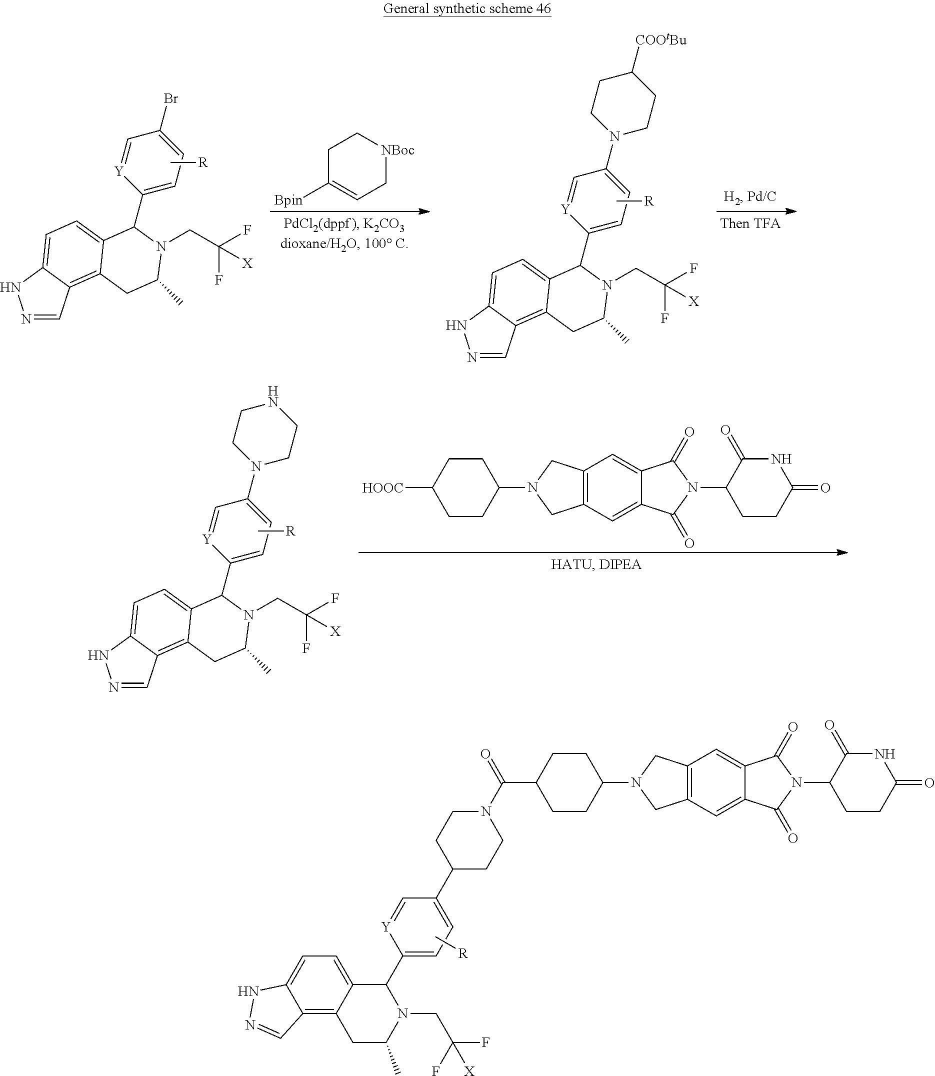

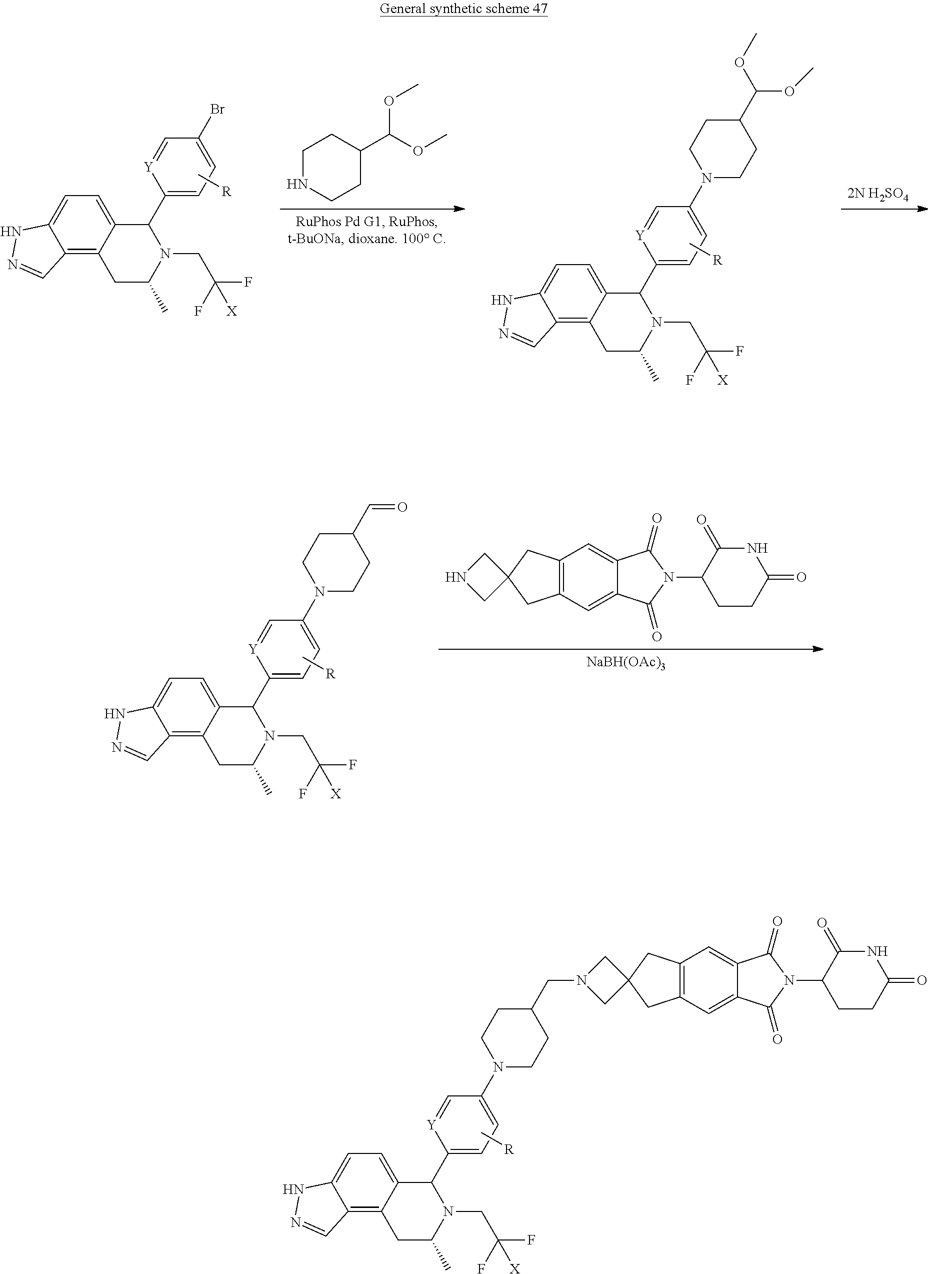

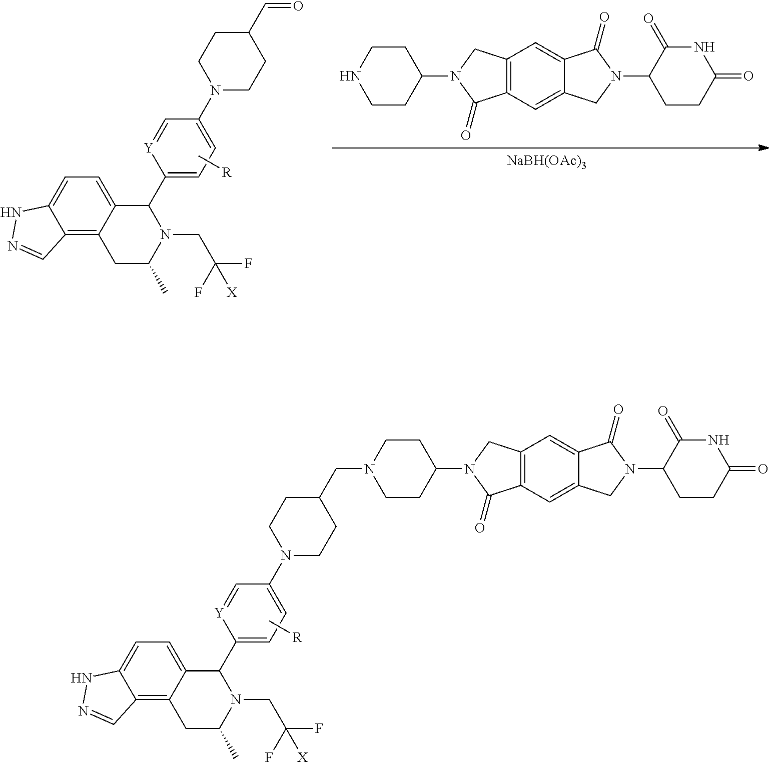

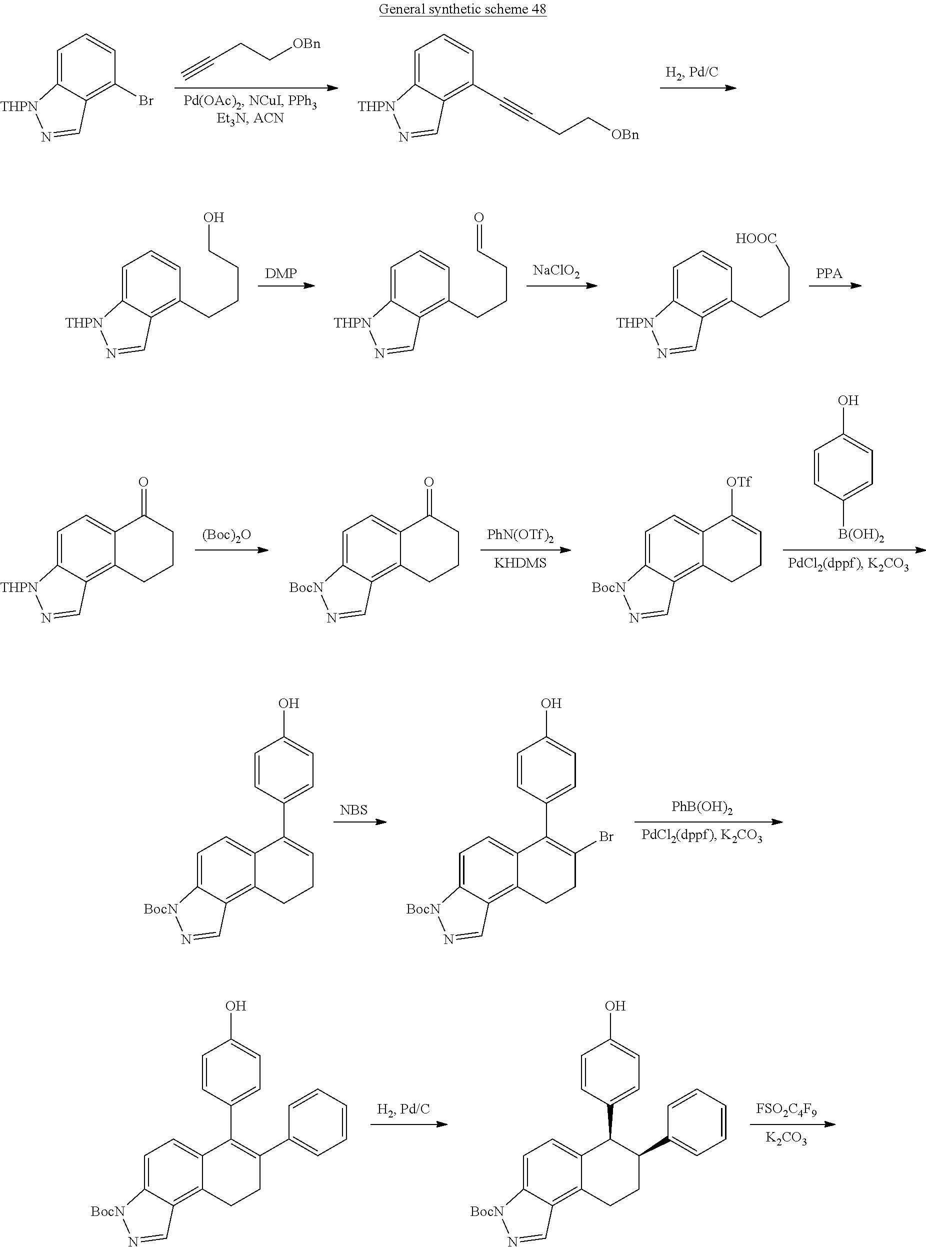

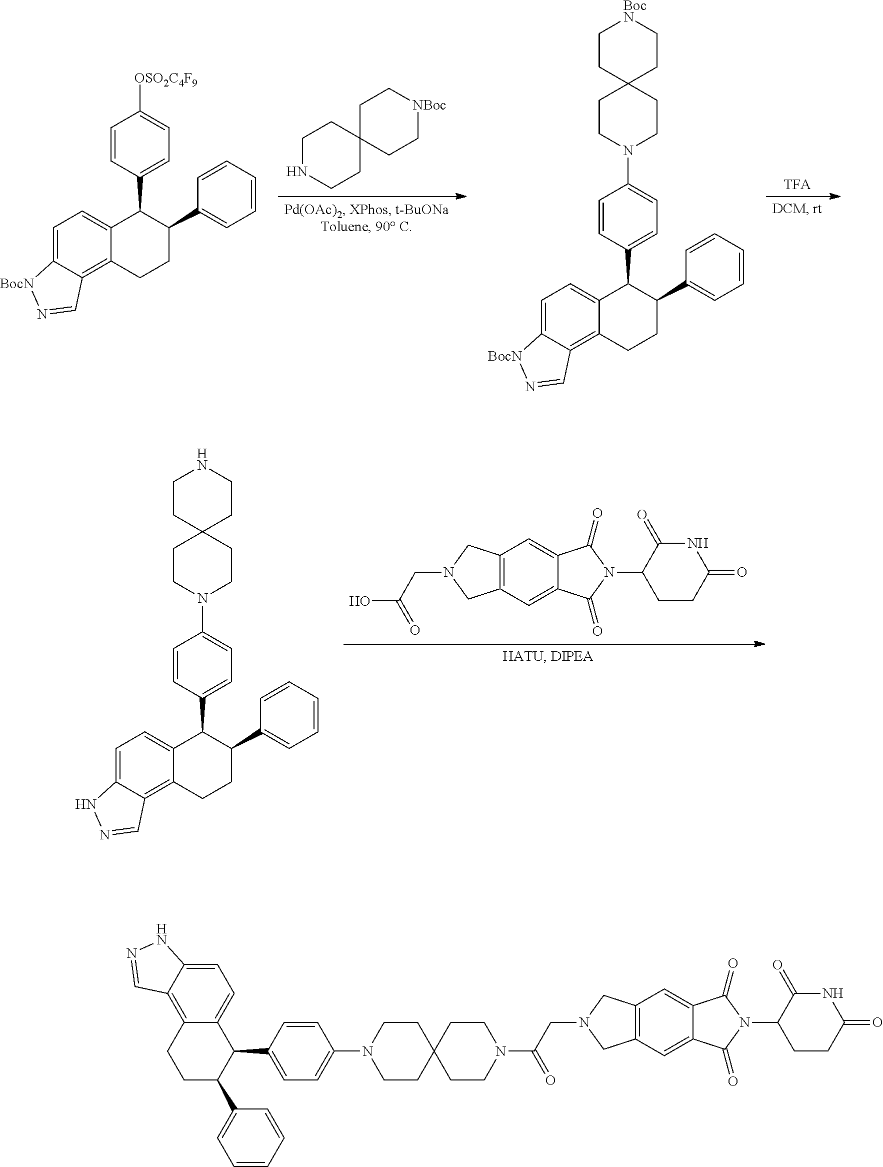

### Example 1. Synthesis of 2-(2,6-dioxopiperidin-3-yl)-6-(2-(6-(4-((1R,2S)-6-hydroxy-2-phenyl-1,2,3,4-tetrahydronaphthalen-1-yl)phenyl)-2,6-diazaspiro[3.3]heptan-2-yl)-2-oxoethyl)-6,7-dihydropyrrolo[3,4-f]isoindole-1,3(2H,5H)-dione (E 8)

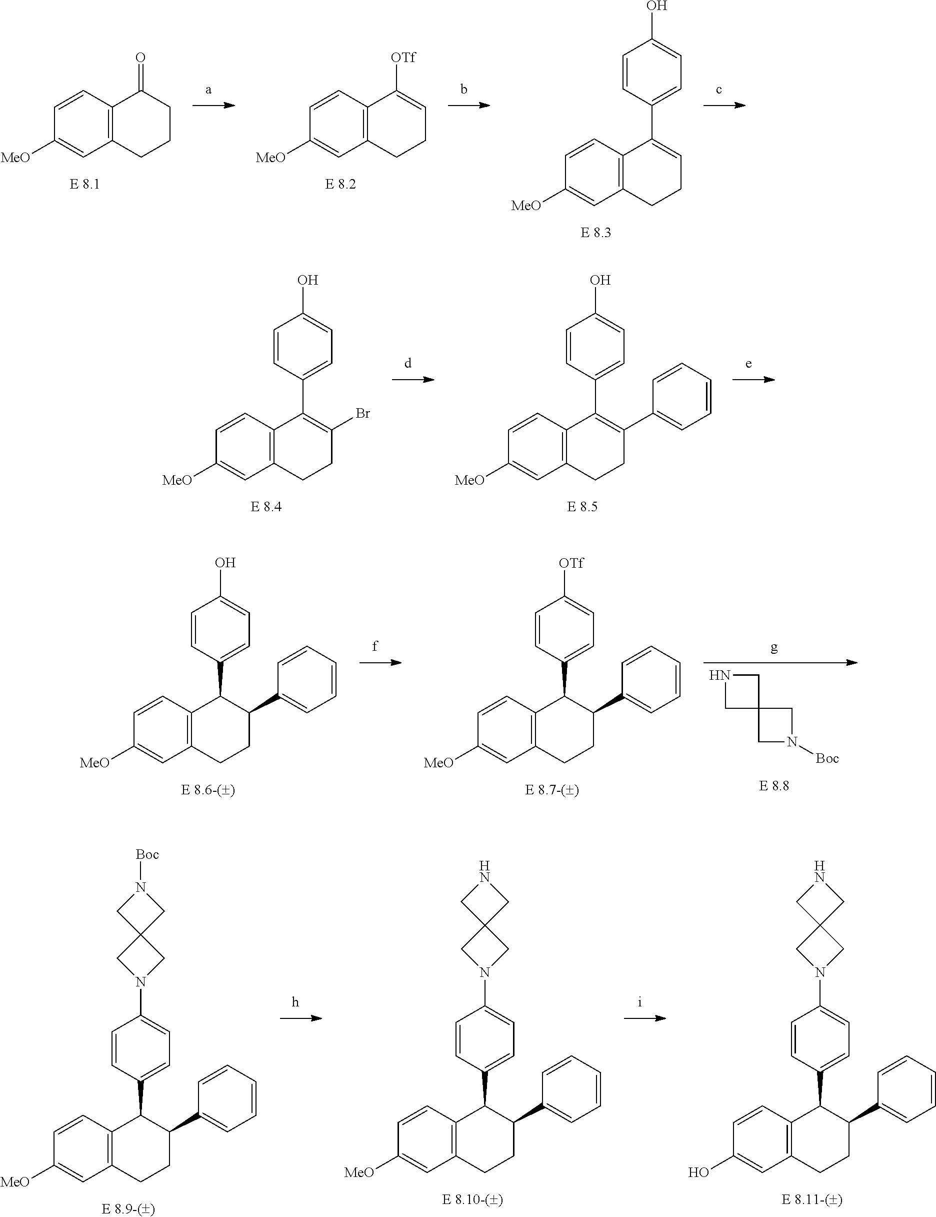

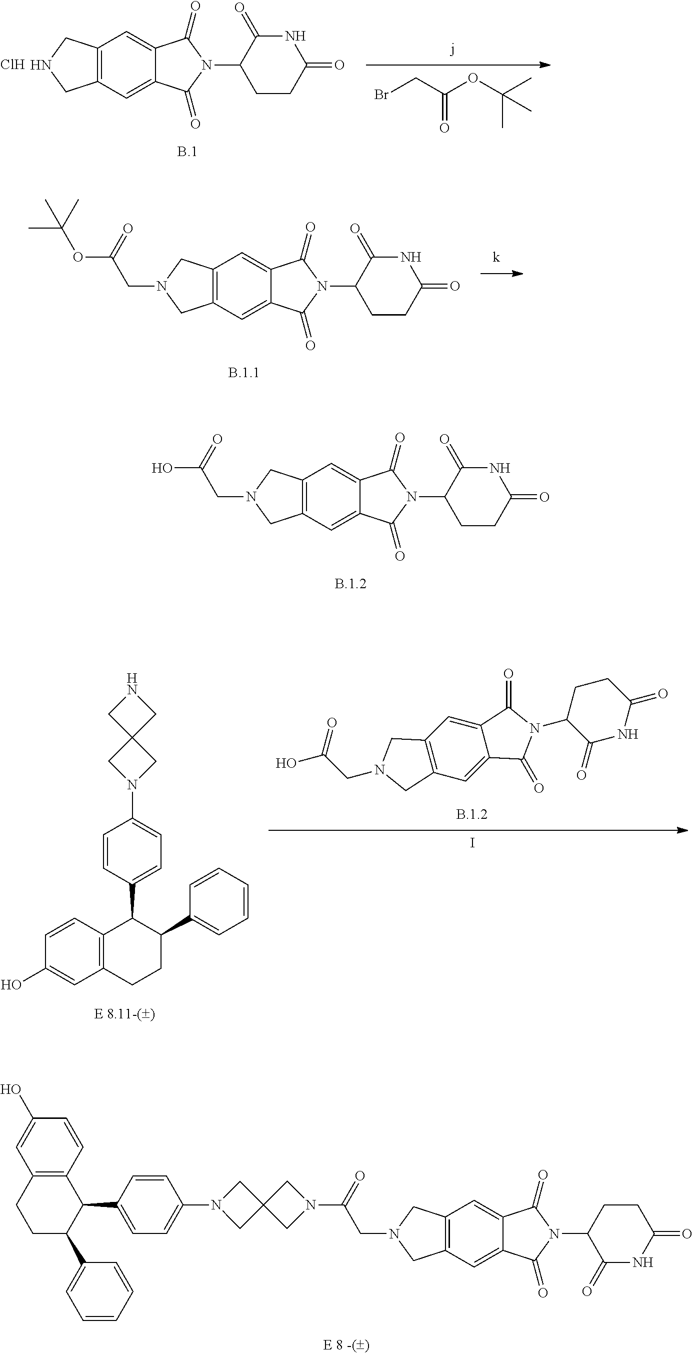

Reagent and conditions: (a) KHMDS, PhN(SO₂CF₃)₂, THF, rt, 82%; (b) (4-Hydroxyphenyl)boronic acid, PdCl₂dppf, K₂CO₃, dioxane, H₂O, 100° C., 75%; (c) NBS, THF, rt, 83%; (d) Phenyl boronic acid, PdCl₂dppf, Cs₂CO₃, dioxane, H₂O, 70° C., 75%; (e) Pd/C, MeOH, 99%; (f) Tf₂O, Et₃N, DCM, 72%; (g) E 8.8, Pd₂(dba)₃, XantPhos, Cs₂CO₃, dioxane; (h) DCM, TFA, 60% in two steps; (i) BBr₃, DCM, 60%; (j) tert-Butyl bromoacetate, DIPEA, DCM, 70%; (k) TFA, DCM; (1) HATU, DIPEA, DMF, 80%.

Preparation of E 8.2: To a mixture of E 8.1 (6 g, 34 mmol, 1 eq.) and PhNTf₂ (14.5 g, 40.6 mmol, 1.2 eq.) in THE (124 mL); KHMDS (0.5 M in toluene, 102 mL, 1.5 eq.) was added into the flask at room temperature. The mixture was then stirred for 15 mins and quenched with saturated NaHCO₃ (100 mL). Water (100 mL) and ethyl acetate (200 mL) were added to the reaction mixture. The organic phase was then dried over magnesium sulfate, filtered, and concentrated under reduced pressure. The residue was purified by silica gel chromatography (eluted with 0-50% EtOAc/hexane) to afford compound E 8.2 (8.6 g, 27.9 mmol, 82%).

Preparation of E 8.3: To a solution of E 8.2 (8.6 g, 27.9 mmol, 1 eq.) and (4-hydroxy phenyl)boronic acid (5.7 g, 41 mmol, 1.5 eq.) in dioxane/H₂O=232 mL/46 mL. K₂CO₃ (7.7 g, 55.8 mmol, 2 eq.) and PdCl₂dppf (2.28 g, 2.79 mmol, 0.1 eq.) were added into the flask under nitrogen. The reaction mixture was stirred at 100° C. for 10 h. TLC showed reaction was completed. Quenched with saturated NaHCO₃ (100 mL), The organic phase was separated. Ethyl acetate (100 mL×3) was added to the mixture, the resulting mixture was washed by brine (50 mL×2). The combined organic phase was dried by MgSO₄. Filtered and concentrated in vacuum. The residue was purified by silica gel chromatography (eluted with 0-50% EtOAc/hexane) to give compound E 8.3 (5.3 g, 21 mmol, 75%).

Preparation of E 8.4: To a solution of E 8.3 (4.8 g, 19 mmol, 1 eq.) in THE (190 mL), NBS (3 g, 17.1 mmol, 0.9 eq.) was added into the flask. The reaction mixture was stirred at room temperature for 10 min. The reaction was quenched with saturated NaHCO₃ (50 mL), The organic phase was separated. Ethyl acetate (50 mL×3) was added to the mixture, the resulting mixture was washed by brine (50 mL). The combined organic phase was dried by MgSO₄. Filtered and concentrated in vacuum. The residue was purified by silica gel chromatography (eluted with 0-50% EtOAc/hexane) to give compound E 8.4 (5.2 g, 15.7 mmol, 83%).

Preparation of E 8.5: A mixture of E 8.4 (5 g, 15.1 mmol, 1 eq.), Phenyl boronic acid (1.94 g, 15.9 mmol, 1.05 eq.), K₂CO₃ (4.14 g, 30 mmol, 2 eq.), PdCl₂dppf (1.22 g, 1.5 mmol, 0.1 eq.) in (dioxane/H₂O=130 mL: 26 mL) were added into the flask under nitrogen. The reaction mixture was stirred at 100° C. for 5 h. After cooling to room temperature, the reaction mixture was extracted with ethyl acetate (50 mL×3) and water. The combined organic layer was washed by brine (50 mL×2). The combined organic phase was dried by MgSO₄. Filtered and concentrated in vacuum. The residue was purified by silica gel chromatography (eluted with 0-100% EtOAc/hexane) to give compound E 8.5 (3.8 g, 11.6 mmol, 75%).

Preparation of E 8.6: To a solution of E 8.5 (3.8 g, 11.6 mmol, 1 eq.) in MeOH (116 mL) 0.38 g Pd/C was added. The reaction mixture was stirred in presence of H₂ atmosphere for 24 h. The reaction was monitored by UPLC-MS. Pd/C was filtered by celite and the filtrated was concentrated in vacuum to get compound E 8.6 (3.8 g, 11.5 mmol, 99%).

Preparation of E 8.7: To a solution of E 8.6 (0.2 g, 0.6 mmol, 1 eq.) in DCM (6 mL), Et₃N (0.25 mL, 1.82 mmol, 3 eq.) and Tf₂O (0.2 mL, 1.21 mmol, 2 eq.) was added dropped into flask at room temperature. The reaction was stirred for 2 h. After that, the reaction was quenched with 5 mL of saturated NaHCO₃, and the organic phase was separated. DCM (5 mL×3) was added to the mixture, the resulting mixture was washed by brine (5 mL). The combined organic phase was dried by MgSO₄ and concentrated in vacuum. The residue was purified by silica gel chromatography (Hexane:ethyl acetate=1:0 to 10:1) to give compound E 8.7 (0.2 g, 0.43 mmol, 72%).

Preparation of E 8.10: A mixture of E 8.7 (1 eq.), E 8.8 (1.5 eq.), Cs₂CO₃ (2.5 eq.), Xantphos (0.3 eq.) and Pd₂(dba)₃ (0.2 eq.) were added in dioxane (c=0.2 mol/L) under N₂. The reaction mixture was stirred at 100° C. for 8 h. After that the reaction was quenched with 5 mL of H₂O, and the organic phase was separated. Ethyl acetate (5 mL×3) was added to the mixture, the resulting mixture was washed by brine (5 mL). The combined organic phase was dried by MgSO₄ and concentrated in vacuum. The reaction was then concentrated and purify by HPLC, followed by deprotection of Boc group by TFA to get the compound E 8.10 in 60% yield.

Preparation of E 8.11: To a solution of E 8.10 (1 eq) in CH₂Cl₂ (c=0.1 mol/L). BBr₃ (2 eq.) was added into the flask at 0° C., then warm to room temperature. The reaction was monitored by TLC, CH₂Cl₂:MeOH=5:1. The reaction was quenched by MeOH, concentrated in vacuum, add some MeCN and purified by HPLC to get compound E 8.11 in 60% yield.

Preparation of B.1.1: To a solution of compound B.1 in CH₂Cl₂ (c=0.2 mol/L), DIPEA (5 eq.) was added at room temperature and stirred for 30 mins. tert-Butyl bromoacetate (1 eq.) was added into flask. The reaction can be monitored by TLC using CH₂Cl₂:MeOH=10:1 as a eluent. The reaction was then concentrated in vacuum and purified by silica gel chromatography CH₂Cl₂ to CH₂Cl₂:MeOH=10:1 to get compound B.1.1 in ˜70% yield.

Preparation of B.1.2: Compound B.1.1 was dissolved into CH₂Cl₂ (c=0.2 mol/L), followed by addition of TFA (10 eq.) into flask. The reaction was monitored by UPLC-MS. Concentrated in vacuum and freeze-dried to get B.1.2.

Preparation of E 8: To a solution of B.1.2 (1.0 eq.) in DMF (c=0.05 mol/L), DIPEA (10 eq.) and HATU (0.9 eq.) were added into the flask and stirred for 10 mins at room temperature. Followed by addition of compound E 8.11 (1 eq.) into flask and stirred for 10 mins. The reaction was monitored by UPLC-MS. It was then quenched by addition of H₂O; followed by purification using HPLC to get the final compound E.8 in 80% yield.

### Example 2. Synthesis of 2-(2,6-dioxopiperidin-3-yl)-6-(2-(7-(4-((1R,2S)-2-(4-fluorophenyl)-6-hydroxy-1,2,3,4-tetrahydronaphthalen-1-yl)phenyl)-2,7-diazaspiro[3.5]nonan-2-yl)acetyl)-6,7-dihydropyrrolo[3,4-f]isoindole-1,3(2H,5H)-dione (E 127)

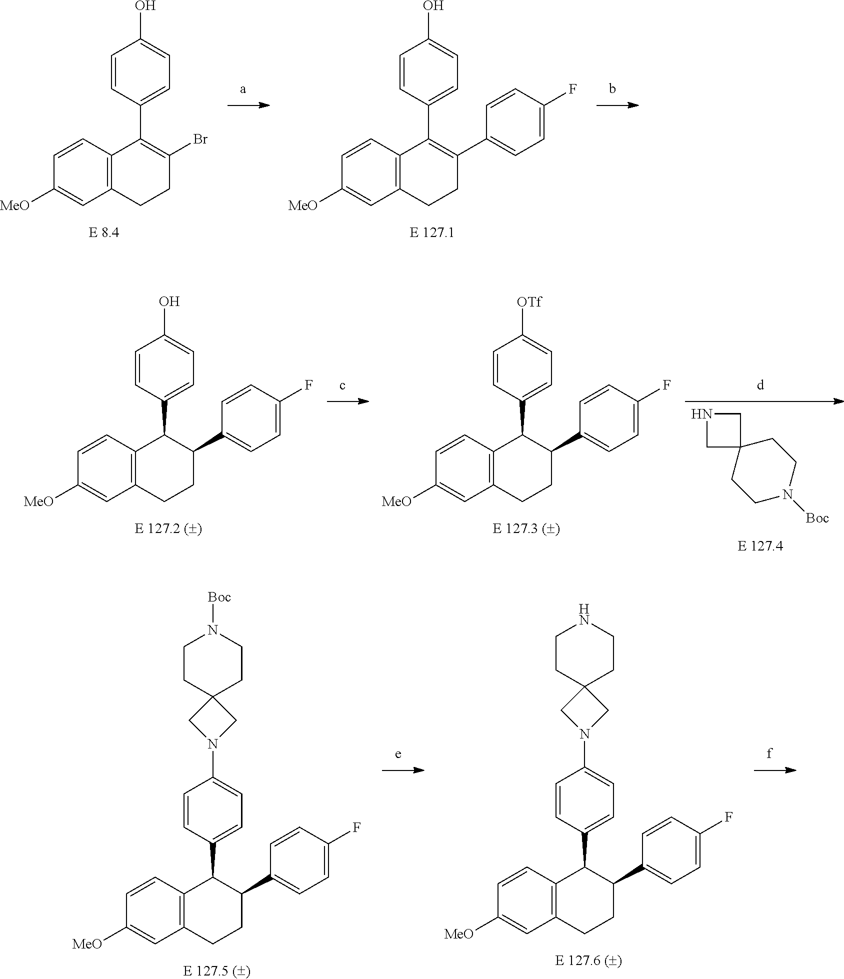

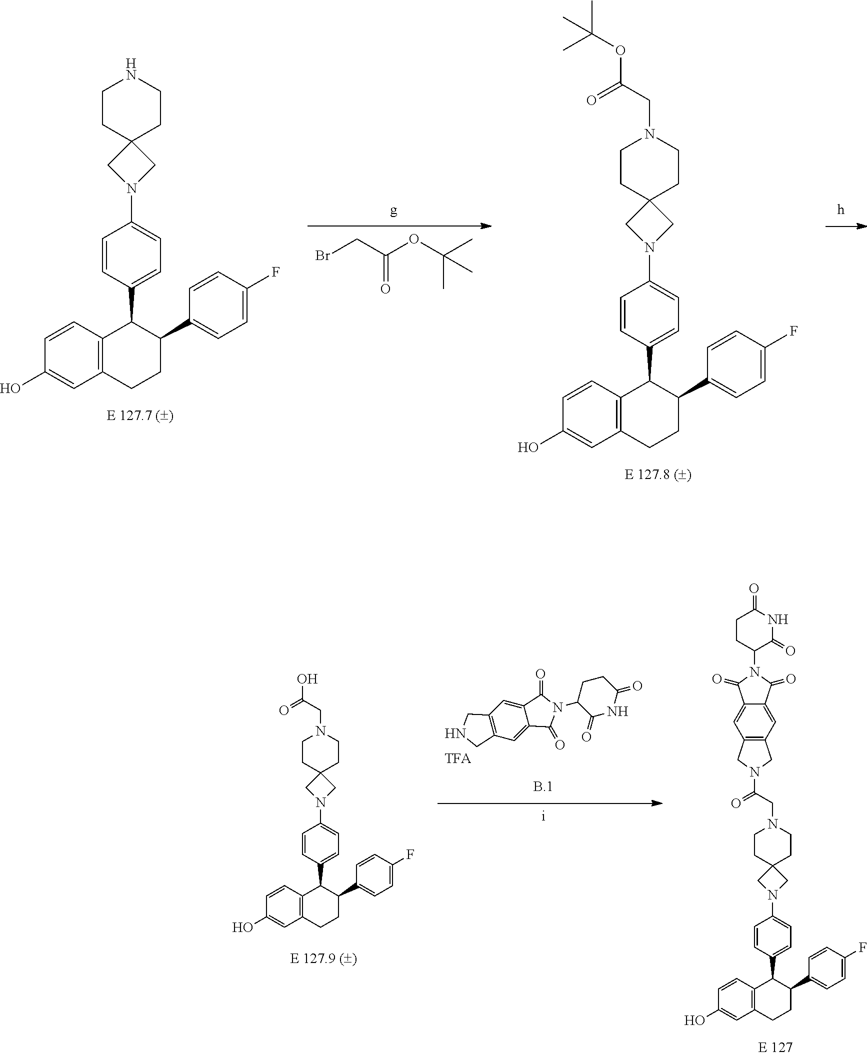

Reagent and conditions: (a) (4-Fluorophenyl)boronic acid, PdCl₂dppf, K₂CO₃, dioxane, H₂O, 70° C., 75%; (b) Pd/C, MeOH, 99%; (c) Tf₂O, Et₃N, DCM, 72%; (d) E 127.4, Pd₂(dba)₃, XantPhos, Cs₂CO₃, dioxane; (e) DCM, TFA, 60% in two steps; (f) BBr₃, DCM, 60%; (g) tert-Butyl bromoacetate, DIPEA, DCM, 60%; (h) TFA, DCM; (i) HATU, DIPEA, DMF, 80%.

Preparation of E 127.1: A mixture of E 8.4 (5 g, 15.1 mmol, 1 eq.), (4-fluorophenyl)boronic acid (2.22 g, 15.9 mmol, 1.05 eq.), K₂CO₃ (4.14 g, 30 mmol, 2 eq.), PdCl₂dppf (1.22 g, 1.5 mmol, 0.1 eq.) in (dioxane/H₂O=130 mL: 26 mL) were added into the flask under nitrogen. The reaction mixture was stirred at 100° C. for 5 h. After cooling to room temperature, the reaction mixture was extracted with ethyl acetate (50 mL×3) and water. The combined organic layer was washed by brine (50 mL×2). The combined organic phase was dried by MgSO₄. Filtered and concentrated in vacuum. The residue was purified by silica gel chromatography (eluted with 0-50% EtOAc/hexane) to give compound E 127.1 (4.01 g, 11.6 mmol, 75%).

Preparation of E 127.2: To a solution of E 127.1 (4.0 g, 11.6 mmol, 1 eq.) in MeOH (116 mL). Pd/C (0.38 g) was added. The reaction mixture was stirred in presence of H₂ atmosphere for 24 h. The reaction was monitored by UPLC-MS. Pd/C was filtered by celite and the filtrated was concentrated in vacuum to get compound E 127.2 (4.0 g, 11.5 mmol, 99%).

Preparation of E 127.3: To a solution of E 127.2 (0.2 g, 0.57 mmol, 1 eq.) in DCM (6 mL), Et₃N (0.25 mL, 1.8 mmol, 3 eq.) and Tf₂O (0.2 mL, 1.2 mmol, 2 eq.) was added dropped into flask at room temperature. The reaction was stirred for 2 h. After that the reaction was quenched with 5 mL of saturated NaHCO₃, and the organic phase was separated. Ethyl acetate (5 mL×3) was added to the mixture, the resulting mixture was washed by brine (5 mL). The combined organic phase was dried by MgSO₄ and concentrated in vacuum. The residue was purified by silica gel chromatography (Hexane:ethyl acetate=1:0 to 10:1) to give compound E 127.3 (0.2 g, 0.43 mmol, 72%).

Preparation of E 127.6: A mixture of E 127.3 (1 eq.), E 127.4 (1.5 eq.), Cs₂CO₃ (2.5 eq.), Xantphos (0.3 eq.) and Pd₂(dba)₃ (0.2 eq.) were added in dioxane (c=0.2 mol/L) under N₂. The reaction mixture was stirred at 100° C. for 8 h. After that the reaction was quenched with 5 mL of H₂O, and the organic phase was separated. Ethyl acetate (5 mL×3) was added to the mixture, the resulting mixture was washed by brine (5 mL). The combined organic phase was dried by MgSO₄ and concentrated in vacuum. The reaction was then concentrated and purify by HPLC, followed by deprotection of Boc group by TFA to get the compound E 127.6 in 60% yield.

Preparation of E 127.7: To a solution of E 127.6 (1 eq.) in CH₂Cl₂ (c=0.1 mol/L). BBr₃ (2 eq.) was added into the flask at 0° C., then warm to room temperature. The reaction was monitored by TLC, CH₂Cl₂:MeOH=5:1. The reaction was quenched by MeOH, concentrated in vacuum, add some MeCN and purified by HPLC to get compound E 127.7 in 60% yield.

Preparation of E 127.8: To a solution of compound E 127.7 in CH₂Cl₂ (c=0.2 mol/L), DIPEA (5 eq.) was added at room temperature and stirred for 5 mins. tert-Butyl bromoacetate (1 eq.) was then added into flask. The reaction was monitored by UPLC-MS. It was then quenched by addition of H₂O; followed by purification using HPLC to get the final compound E 127.8 in ˜60% yield.

Preparation of E 127.9: Compound E 127.8 was dissolved into CH₂Cl₂ (c=0.2 mol/L), followed by addition of TFA (10 eq.) into flask. The reaction was monitored by UPLC-MS. Concentrated in vacuum and freeze-dried to get E 127.9.

Preparation of E 127: To a solution of E 127.9 (0.9 eq.) in DMF (c=0.05 mol/L), DIPEA (10 eq.) was added into the flask at room temperature. HATU (0.9 eq.) was added into flask and stirred for 10 mins. Followed by addition of compound B.1 (1 eq.) into flask and stirred for 10 mins. The reaction was monitored by UPLC-MS. It was then quenched by addition of H₂O; followed by purification using HPLC to get the final compound E 127 in 80% yield.

### Example 3. Synthesis of 2-(2,6-dioxopiperidin-3-yl)-6-(2-(6-(4-(8-(4-fluorophenyl)-3-hydroxy-6,7-dihydro-5H-benzo[7]annulen-9-yl)phenyl)-2,6-diazaspiro[3.3]heptan-2-yl)acetyl)-6,7-dihydropyrrolo[3,4-f]isoindole-1,3(2H,5H)-dione (E 49)

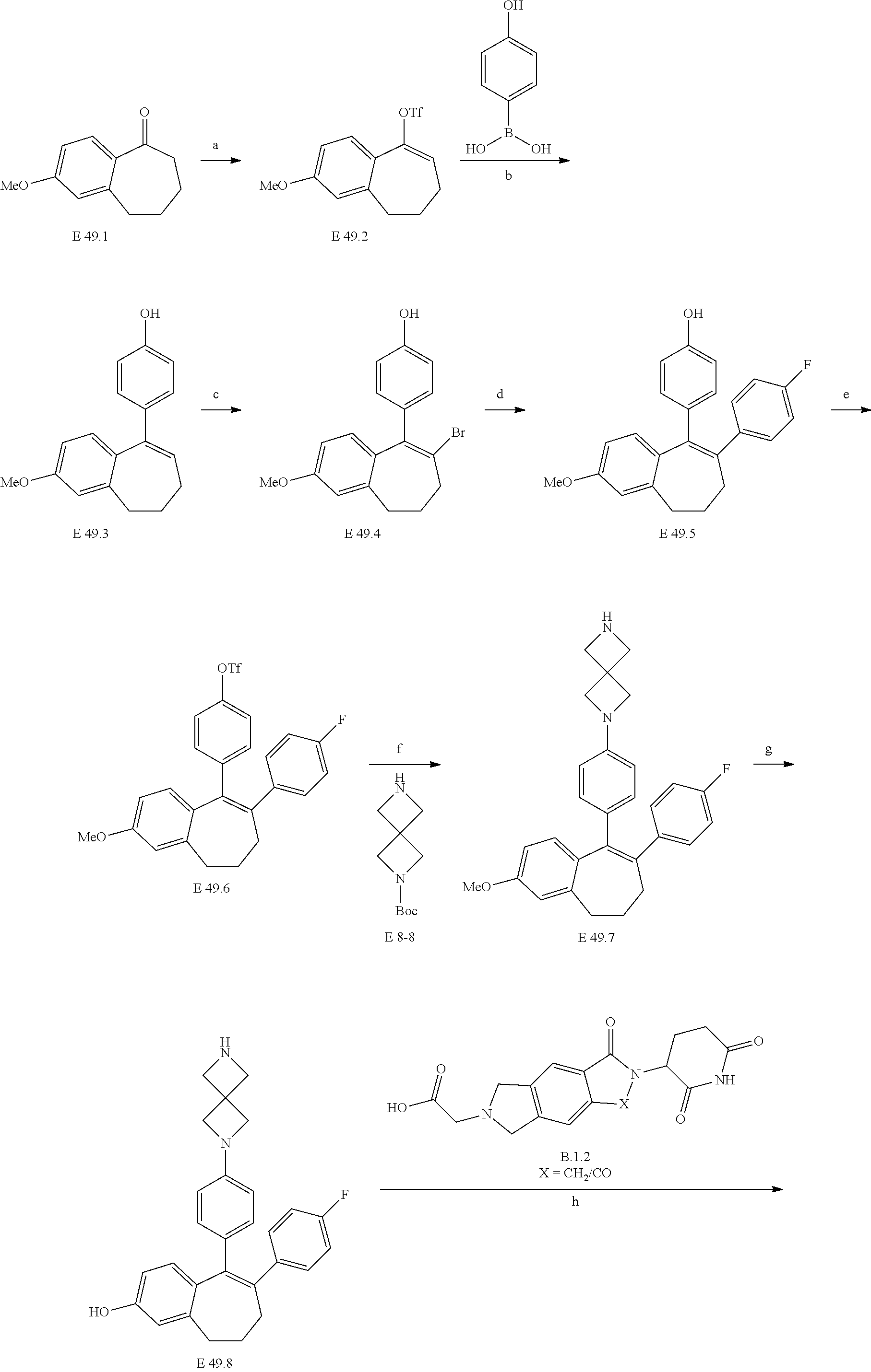

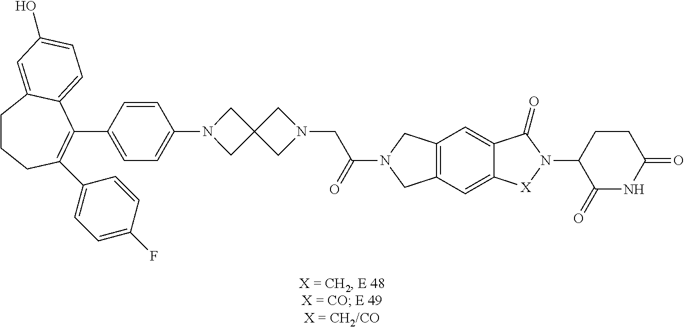

Reagent and conditions: (a) Trifluoromethanesulfonic anhydride, pyridine, DCM, rt, 90%; (b) PdCl₂dppf, Cs₂CO₃, dioxane, H₂O, 90° C., 90%; (c) Pyridinium tribromide, THF, rt, 70%; (d) (4-Fluorophenyl)boronic acid, PdCl₂dppf, Cs₂CO₃, dioxane, H₂O, 90° C., 80%; (e) Tf₂O, Et₃N, DCM, 72%; (f) (i) Pd₂(dba)₃, XantPhos, Cs₂CO₃ (2.5 eq.), dioxane, (ii) DCM, TFA (g) BBr₃, DCM, 50% in two steps; (h) HATU, DIPEA, DMF, 80%.

Preparation of E 49.2: To a solution of E 49.1 (10 mmol) in CH₂Cl₂ (100 mL) was added pyridine (1.26 mL, 14.98 mmol) and trifluoromethanesulfonic anhydride (3.39 mL, 19.97 mmol) dropwise under argon. The reaction mixture was stirred at room temperature for 16 h, and ice (200 g) and CH₂Cl₂ (200 mL) were added. The phases were separated, the aqueous phase was washed with CH₂Cl₂, and the gathered organic phases were dried over magnesium sulfate, filtered, and evaporated under reduced pressure. The residue was purified by silica gel chromatography (eluted with 0-50% EtOAc/hexane) to afford the title compound E 49.2 (3.65 g, 90%) as an orange oil.

Preparation of E 49.3: To a mixture of E 49.2 (1 mmol), (4-hydroxyphenyl)boronic acid (1.1 mmol), and PdCl₂dppf. DCM (0.1 mmol) in dioxane (5 mL) was added dropwise an aq. solution of 1.5 M cesium carbonate (2 mmol). The reaction mixture was stirred for 5 h at 90° C. work up was done using water and ethyl acetate. The organic phase was dried over magnesium sulfate, filtered, and concentrated under reduced pressure. The residue was purified by flash chromatography (eluted with 0-50% EtOAc/hexane) to afford E 49.3 in 90% yield.

Preparation of E 49.4: To a solution of E 49.3 (1 mmol) in THE (4 mL) was added pyridinium tribromide (1 mmol). The reaction mixture was stirred for 24 h at room temperature. Water was added, then pH was adjusted to 8 with an aqueous solution of sodium bicarbonate. Ethyl acetate was added in the reaction mixture. After decantation, the organic phase was dried over magnesium sulfate, filtered, and concentrated under reduced pressure. The residue was The residue was purified by flash chromatography eluted with 0-50% EtOAc/hexane to afford E 49.4 in 70% yield.

Intermediate E 49.5 was obtained from E 49.4 in 80% yield by following the protocol for E 49.3.

Preparation of E 49.6: To a solution of E 49.5 (1 mmol) in DCM (4 mL) was added Tf₂O (2 mmol) and Et₃N (4 mmol). The reaction mixture was stirred for 12 h at room temperature. work up was done using water and DCM. The organic phase was dried over magnesium sulfate, filtered, and concentrated under reduced pressure. The residue was purified by flash chromatography eluting with eluted with 0-50% EtOAc/hexane to obtained E 49.6 in 72% yield.

Preparation of E 49.7: A mixture of E 49.6 (1 eq.), E 8.8 (1.5 eq.), Cs₂CO₃ (2.5 eq.), XantPhos (0.3 eq.) and Pd₂(dba)₃ (0.2 eq.) were added in dioxane (c=0.2 mol/L) under N₂. The reaction mixture was stirred at 100° C. for 8 h. After that the reaction was quenched with 5 mL of H₂O, and the organic phase was separated. Ethyl acetate (5 mL×3) was added to the mixture, the resulting mixture was washed by brine (5 mL). The combined organic phase was dried by MgSO₄ and concentrated in vacuum. The reaction was then concentrated and purify by HPLC and freeze-dried to get the TFA salt of compound E 49.7.

Preparation of E 49.8: To a solution of E 49.7 (1 eq.) in CH₂Cl₂ (c=0.1 mol/L). BBr₃ (2 eq.) was added into the flask at 0° C., then warm to room temperature. The reaction was monitored by UPLC-MS. After completion the reaction was quenched by MeOH, concentrated in vacuum, and purified by HPLC to get compound E 49.8 in 50-60% yield.

Preparation of E 49: To a solution of B.1.2 (0.9 eq.) in DMF (c=0.05 mol/L), DIPEA (10 eq.) and HATU (0.9 eq.) were added into the flask and stirred for 10 mins at room temperature. Followed by addition of compound E 49.8 (1 eq.) into flask and stirred for 10 mins. The reaction was monitored by UPLC-MS. After completion the reaction was quenched by H₂O; followed by purification using HPLC to get the final compound E 49 in 80% yield.

### Example 4. Synthesis of 2-(2,6-dioxopiperidin-3-yl)-5,6,7,8-tetrahydro-1H-pyrrolo[3,4-g]isoquinoline-1,3(2H)-dione (A.1)

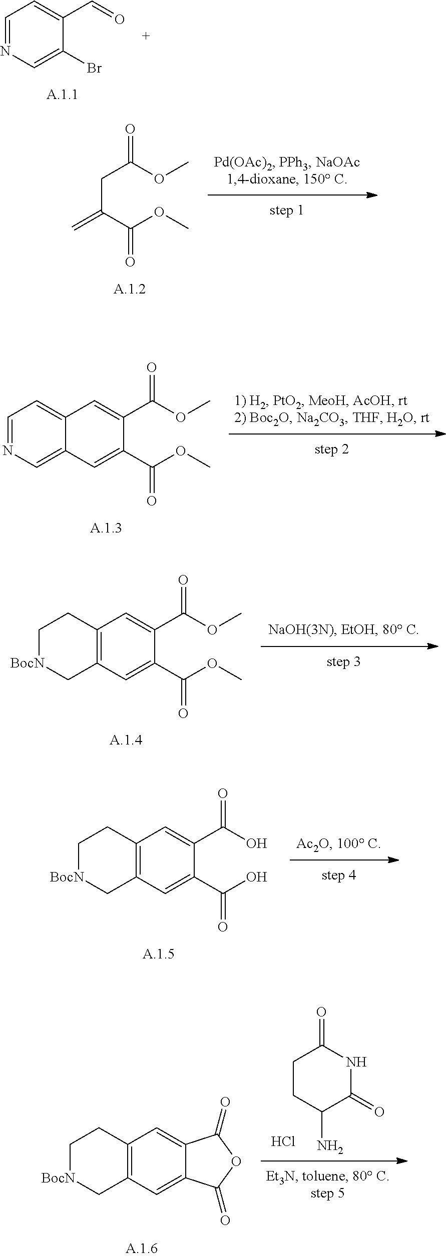

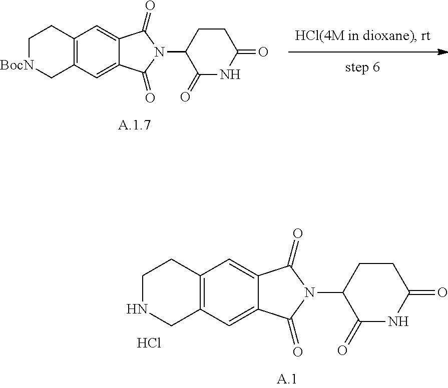 [SMILES: CC(C)(C)OC(=O)N1CCc2cc3c(cc2C1)C(=O)N(C1CCC(=O)NC1=O)C3=O ; Cl ; O=C1CCC(N2C(=O)c3cc4c(cc3C2=O)CNCC4)C(=O)N1]

### Step 1: Synthesis of dimethyl isoquinoline-6,7-dicarboxylate (A.1.3)

A mixture of 3-bromopyridine-4-carbaldehyde (A.1.1, 0.093 g, 0.5 mmol), dimethyl itaconate (A.1.2, 0.079 g, 0.5 mmol), Pd(OAc)₂ (0.0056 g, 0.025 mmol), PPh₃ (0.013 g, 0.05 mmol) and NaOAc (0.123 g, 1.5 mmol) in dioxane (10 mL) was placed in a 50 mL pressure vessel. After the system was flushed with argon, the reaction mixture was allowed to react at 150° C. for 24 h, and then the reaction mixture was cooled to room temperature. The reaction mixture was filtered through celite to eliminate inorganic salts and washed by ethyl acetate. Removal of the solvent left a crude mixture which was purified by flash chromatography on silica gel (ethyl acetate-hexane) to give dimethyl isoquinoline-6,7-dicarboxylate (A.1.3, 0.082 g, 67%).

### Step 2: Synthesis of 2-(tert-butyl) 6,7-dimethyl 3,4-dihydroisoquinoline-2,6,7(1H)-tricarboxylate (A.1.4)

Compound A.1.3 (279.6 mg, 1.14 mmol) was dissolved in mixture solvent of methanol (4 mL) and acetic acid (0.2 mL). PtO₂ (30 mg) was added, and the reaction mixture was stirred under hydrogen at room temperature for 4 h. The reaction mixture was filtered through Celite®. The filtrate was collected and concentrated under reduced pressure to give the crude product.

The crude product was dissolved in mixture of THE (4 mL) and water (1 mL), and Na₂CO₃ (500 mg) and Boc₂O (500 mg, 2.28 mmol) were added to the mixture. The reaction mixture was stirred at room temperature for 2 h. The reaction mixture was concentrated under reduced pressure to remove the THF, and the crude mixture dissolved in water (5 mL) and ethyl acetate (10 mL). The organic layer was separated, washed with water and brine, dried (MgSO₄), concentrated under reduced pressure, and purified by flash chromatography on silica gel (ethyl acetate-hexane) to give compound A.1.4 (130 mg).

### Step 3: Synthesis of 2-(tert-butoxycarbonyl)-1,2,3,4-tetrahydroisoquinoline-6,7-dicarboxylic acid (A.1.5)

3N NaOH (0.37 mL, 1.12 mmol) was added to a solution of compound A.1.4 (130 mg, 0.37 mmol) in EtOH (3.7 mL) and the resulting mixture heated at 80° C. for 2 h. The reaction was concentrated under reduced pressure and the crude mixture dissolved in water (5 mL) and ethyl acetate (10 mL) and then acidified using 1N HCl to pH˜4 in an ice bath. The organic layer was separated and the aqueous layer was extracted with ethyl acetate two more times. The combined the organic layers were washed with brine (10 mL), dried (MgSO₄), and concentrated under reduced pressure. The crude product was used in the next step without further purification.

### Step 4: Synthesis of tert-butyl 1,3-dioxo-1,5,7,8-tetrahydrofuro[3,4-g]isoquinoline-6(3H)-carboxylate (A.1.6)

Compound A.1.5 (the crude product from step 3) was dissolved in acetic anhydride (2 mL) and the reaction mixture was stirred at 100° C. for 3 h. The reaction mixture was cooled to room temperature, and 10 mL ethyl acetate was added. The reaction mixture was washed with water and brine, dried (MgSO₄), concentrated under reduced pressure, and purified by flash chromatography on silica gel (ethyl acetate-hexane) to give compound A.16 (123.1 mg).

### Step 5: Synthesis of tert-butyl 2-(2,6-dioxopiperidin-3-yl)-1,3-dioxo-1,2,3,5,7,8-hexahydro-6H-pyrrolo[3,4-g]isoquinoline-6-carboxylate (A.1.7)

Compound A.1.6 (123.1 mg, 0.41 mmol), 3-aminopiperidine-2,6-dione (73.5 mg, 0.45 mmol) and Et₃N (0.17 mL, 1.23 mmol) were added to toluene (5 mL). The reaction mixture was stirred at 80° C. for 3 h and then cooled to room temperature. The reaction was concentrated under reduced pressure and the crude mixture dissolved in water (5 mL) and ethyl acetate (10 mL). The organic layer was separated, washed with water and brine, dried (MgSO₄), concentrated under reduced pressure, and purified by flash chromatography (ethyl acetate-hexane) to give the desired compound A.1.7.

### Step 6: Synthesis of 2-(2,6-dioxopiperidin-3-yl)-5,6,7,8-tetrahydro-1H-pyrrolo[3,4-g]isoquinoline-1,3(2H)-dione (A.1)

Compound A.1.7 (102.1 mg, 0.24 mmol) was added to 1 mL HCl (4M in 1,4-dioxane), and the mixture reaction mixture was stirred at room temperature for 2 h. The 1,4-dioxane was removed under reduced pressure to give compound A.1 as the HCl salt.

### Example 5. Synthesis of 2-(2,6-dioxopiperidin-3-yl)-6,7-dihydropyrrolo[3,4-f]isoindole-1,3(2H,5H)-dione (B.1)

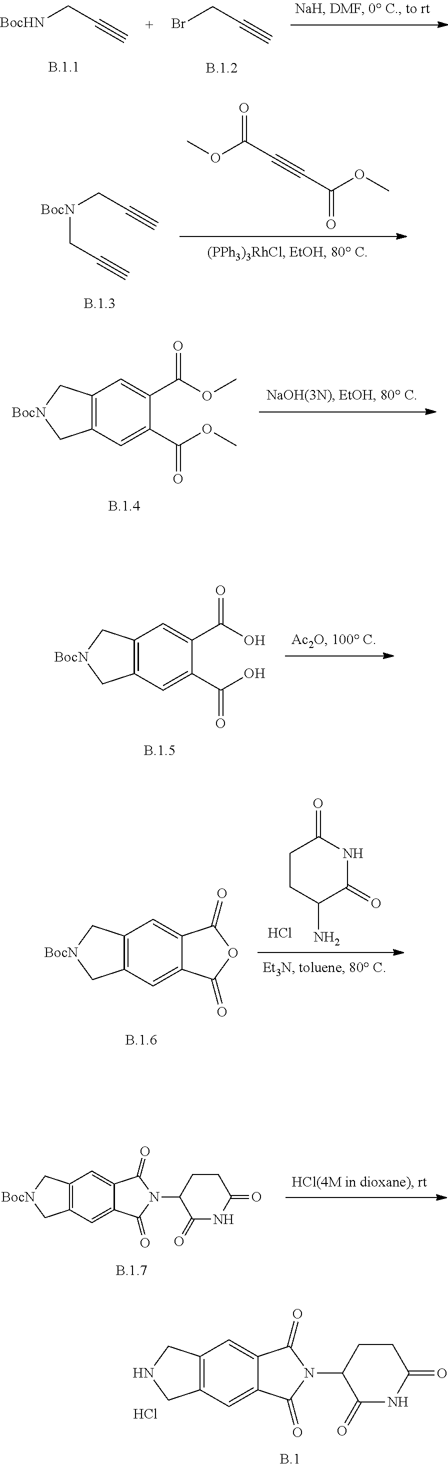

### Step 1: Synthesis of tert-butyl di(prop-2-yn-1-yl)carbamate (B.1.3)

A solution of N-(tert-butyloxy)carbonyl propargylamine (B.1.1; 33.36 g, 215 mmol) in 50 mL of DMF was treated portionwise (4 times) with 60% NaH (10.4 g) at 0° C. After stirring for 30 min at 25° C., 39 mL of an 80% solution of propargyl bromide) in toluene was added. The reaction mixture was stirred for an additional 5 h at 25° C., and then quenched with the addition of ice-water. The mixture was extracted with Et₂O (3×200 mL), and the combined extracts were washed with saturated aqueous NaCl, dried (Na₂SO₄), concentrated in vacuo, and purified by flash chromatography on silica gel (ethyl acetate-hexane) to give compound B.1.3.

### Step 2: Synthesis of 2-(tert-butyl) 5,6-dimethyl isoindoline-2,5,6-tricarboxylate (B.1.4)

A solution of compound B.1.3 (10.4 g, 53.9 mmol) and dimethyl acetylenedicarboxylate (30.7 g, 216 mmol) in 110 mL of absolute EtOH was degassed by bubbling N₂ through the solution for 10 min. To this solution was added 1.0 g (0.02 equiv) of Wilkinson's catalyst ((Ph₃P)₃RhCl) at 25° C. After being warmed at reflux for 18 h, the reaction mixture was cooled to 25° C. and concentrated in vacuo. The resulting brown residue was diluted in 200 mL of Et₂O, and the precipitate was removed by filtration over Celite©. The filtrate was concentrated and the crude product purified by column chromatography on silica gel (20% EtOAc/hexane) to give 4.60 g (26%) of B.1.4.

The remaining steps for synthesizing compound B.1 (as the HCl salt) are essentially the same as Steps 3-6 described above in EXAMPLE 4 (compound A.1).

### Example 6. Synthesis of 2-(2,6-dioxopiperidin-3-yl)-1,3-dioxo-1,2,3,5,6,7-exahydro-cyclopenta[f]isoindole-6 carbaldehyde (C.1)

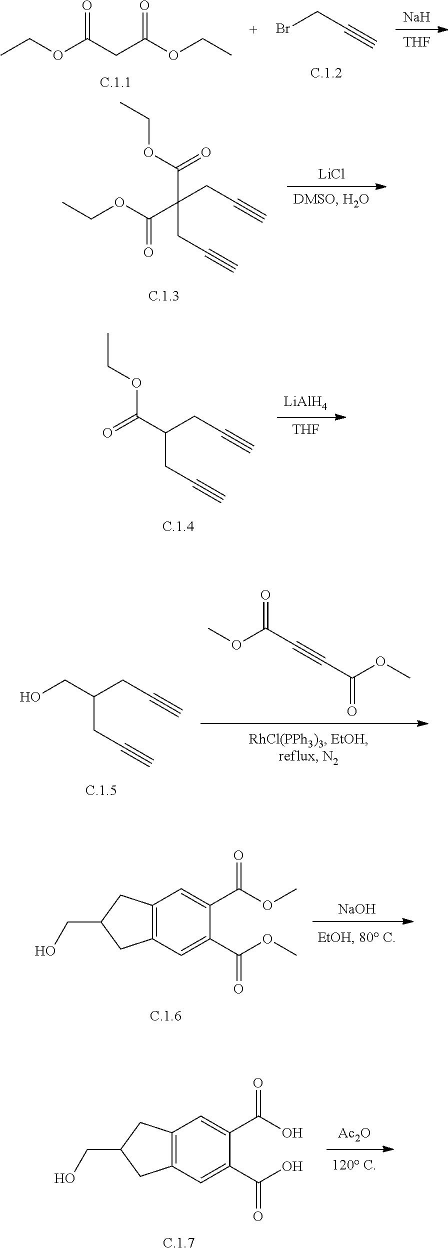

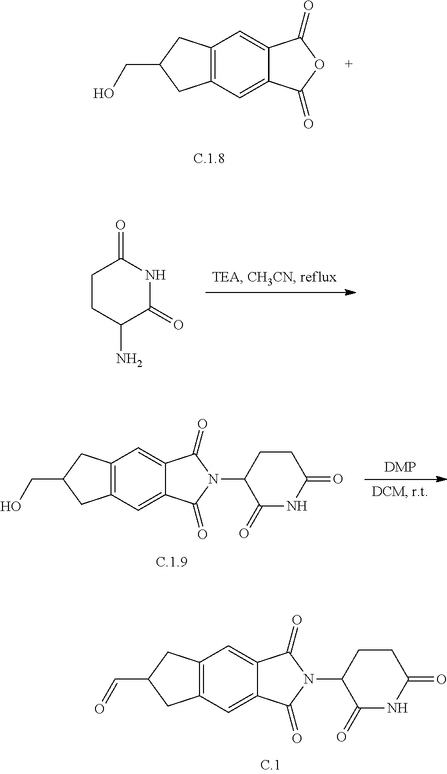

### Step 1: Synthesis of diethyl 2,2-di(prop-2-yn-1-yl)malonate (C.1.3)

To a suspension of sodium hydride (60% wt in mineral oil, 4.22 g, 105.5 mmol) in dry THF (100 mL) stirring at −10° C., dimethyl malonate (6.0 mL, 52.5 mmol) was added dropwise over 10 min. The reaction mixture was stirred at −10° C. for 5 min, and then propargyl bromide (80% wt. in toluene, 12.0 mL, 107.7 mmol) was added dropwise. The reaction mixture was warmed to 25° C. and stirred for 20 h. The reaction mixture was then poured into H₂O (50 mL) and Et₂O (50 mL), and the layers were separated. The aq layer was extracted with Et₂O (3×50 mL). The combined organic phases were washed with brine (50 mL), dried over MgSO₄, filtered, and concentrated on a rotary evaporator leaving a white solid. The solid was recrystallized from ethyl acetate and hexanes resulting in 9.44 g of a crystalline white solid (C.1.3, 84% yield).

### Step 2: Synthesis of ethyl 2-(prop-2-yn-1-yl)pent-4-ynoate (C.1.4)

Dimethyl 2,2-di(2-propynyl)malonate (4.70 g, 22.6 mmol) and lithium chloride (2.95 g, 69.7 mmol) were dissolved in a solution of H₂O (1.0 mL, 55.5 mmol) and DMSO (40 mL). This solution was then heated to reflux for 1 h. After cooling, the reaction mixture was poured into CHCl₃ (40 mL) and H₂O (40 mL). The layers were separated and the aq layer was extracted with CHCl₃ (3×40 mL). The combined organic layers were washed with H₂O (50 mL) and brine (50 mL), dried, filtered through silica gel, and concentrated, leaving a yellow oil. The crude oil was purified by flash chromatography on a silica gel column using 20% EtOAc in hexanes as the eluent resulting in 3.06 g of a pale yellow oil (90% yield).

### Step 3: Synthesis of ethyl 2-(prop-2-yn-1-yl)pent-4-yn-1-ol (C.1.5)

To a suspension of lithium aluminum hydride (1.25 g, 33.0 mmol) in dry THE (40 mL) stirring at −10° C. was added a solution of methyl 2-(2-propynyl)-4-pentynoate (3.06 g, 20.4 mmol) in dry THE (10 mL). The reaction mixture was allowed to warm to 25° C. and stirred for 12 h. The reaction mixture was then quenched through the dropwise addition of H₂O (1.25 mL), an aq 10% NaOH solution (1.25 mL), and then additional H₂O (3.75 mL). The reaction mixture was then stirred for 30 min until the suspended solids turned white. The mixture was then filtered, and the solids were washed with diethyl ether (100 mL). The resulting solution was concentrated on a rotary evaporator yielding a pale yellow oil. The crude oil was purified by flash chromatography on a silica gel column using 10% EtOAc in hexanes as the eluent, resulting in 1.95 g of a clear oil (78% yield).

### Step 4: Synthesis of dimethyl 2-(hydroxymethyl)-2,3-dihydro-1H-indene-5,6-dicarboxylate (C.1.6)

A solution of C.1.5 and dimethyl acetylenedicarboxylate (30.7 g, 216 mmol) in 110 mL of absolute EtOH was degassed by bubbling N₂ through the solution for 10 min. To this was added 1.0 g (0.02 equiv) of Wilkinson's catalyst ((Ph₃P)₃RhCl) at 25° C. After being warmed at reflux for 18 h, the reaction mixture was cooled to 25° C. and then concentrated in vacuo. The resulting brown residue was diluted in 200 mL of Et₂O, and the precipitate was removed by filtration over Celite®. The filtrate was concentrated and the crude product purified by column chromatography (20% EtOAc/hexane) to give 4.60 g (26%) of compound C.1.6.

### Step 5: Synthesis of 2-(hydroxymethyl)-2,3-dihydro-1H-indene-5,6-dicarboxylic acid (C.1.7)

NaOH (3N) was added to a solution of C.1.6 in EtOH and stirred at 80° C. for 4 h. Then the EtOH was removed under reduced pressure, the pH was adjusted to acidity with 2M HCl and the mixture was extracted with EtOAc. The solvent was removed to afford the product C.1.7 which was used without further purification.

### Step 6: Synthesis of 6-(hydroxymethyl)-6,7-dihydro-1H-indeno[5,6-c]furan-1,3(5H)-dione (C.1.8)

The mixture of C.1.7 in Ac₂O was stirred at 120° C. for 6 hours. All volatiles were removed and the residue was chromatographed on silica gel to afford C.1.8.

### Step 7: Synthesis of 2-(2,6-dioxopiperidin-3-yl)-6-(hydroxymethyl)-6,7-dihydrocyclopenta[f]isoindole-1,3(2H,5H)-dione (C.1.9)

To a solution of C.1.8 and 3-aminopiperidine-2,6-dione in toluene was added TEA (3 eq.). The mixture was stirred at reflux for 8 hours. All volatiles were removed and the residue was chromatographed on silica gel to afford C.1.9.

### Step 8: Synthesis of 2-(2,6-dioxopiperidin-3-yl)-1,3-dioxo-1,2,3,5,6,7-hexahydrocyclopenta[f]isoindole-6-carbaldehyde (C.1)

To a solution of C.1.9 in DCM was added DMP (1.2 eq.). The reaction mixture was stirred at reflux for 4 hours. All volatiles were removed and the residue was chromatographed on silica gel to afford 2-(2,6-dioxopiperidin-3-yl)-1,3-dioxo-1,2,3,5,6,7-hexahydrocyclopenta[f]isoindole-6-carbaldehyde (C.1). ESI-MS: 326.09.

### Example 7. Synthesis of 2-(2,6-dioxopiperidin-3-yl)-5,7-dihydrocyclopenta[f]isoindole-1,3,6(2H)-trione (D.1)

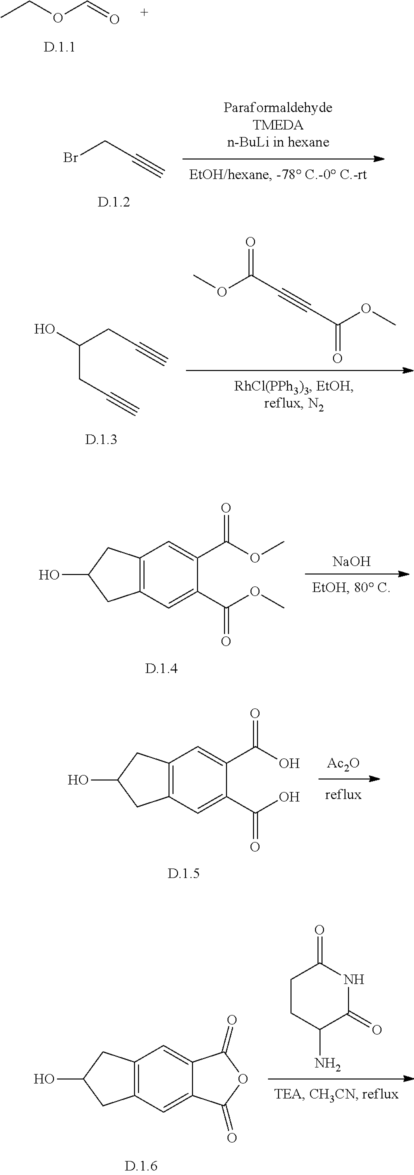

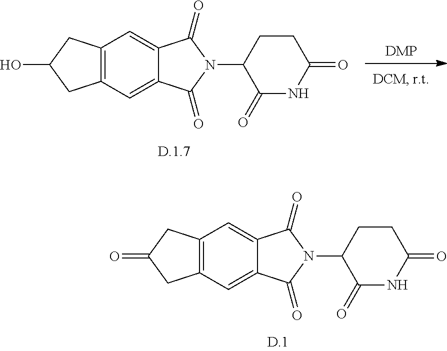

### Step 1: Synthesis of hepta-1,6-diyn-4-ol (D.1.3)

To a solution of n-BuLi in hexane (6.2 eq., 75 mL) in Et₂O/hexane (100 mL) was added TMEDA (7.5 mL) and D.1.1 (3.1 eq.) by dropwise at −78° C. The reaction mixture was stirred at −78° C. for 40 min, and then D.1.2 in THF (20 mL) was added dropwise with 10 min. The reaction mixture was warmed to 25° C. and stirred for 2 h. The reaction mixture was then cooled to −78° C. and added 20 mL THF and Paraformaldehyde (13.5 g) in one portion. Then, the mixture was stirred at r.t. overnight. The mixture was added ice-cold NH₄Cl solution and extracted with Et₂O (3×50 mL). The combined organic phases were washed with brine (50 mL), dried over MgSO₄, filtered, and concentrated on a rotary evaporator leaving a white solid. The solid was recrystallized from ethyl acetate and hexanes resulting in D.1.3.

### Step 2: Synthesis of dimethyl 2-hydroxy-2,3-dihydro-1H-indene-5,6-dicarboxylate (D.1.4)

A solution of D.1.3 and dimethyl acetylenedicarboxylate (30.7 g, 216 mmol) in 110 mL of absolute EtOH was degassed by bubbling N₂ through the solution for 10 min. To this was added 1.0 g (0.02 equiv) of Wilkinson's catalyst ((Ph₃P)₃RhCl) at 25° C. After being warmed at reflux for 18 h, the reaction mixture was cooled to 25° C. and then concentrated in vacuo. The resulting brown residue was diluted in 200 mL of Et₂O, and the precipitate was removed by filtration over Celite®. The filtrate was concentrated and the crude product purified by column chromatography (20% EtOAc/hexane) to give 4.60 g (26%) of compound D.1.4.

### Step 3: Synthesis of 2-hydroxy-2,3-dihydro-1H-indene-5,6-dicarboxylic acid (D.1.5)

NaOH (3N) was added to a solution of D.1.4 in EtOH and stirred at 80° C. for 4 h. Then the EtOH was removed under reduced pressure, the pH was adjusted to acidity with 2M HCl and the mixture was extracted with EtOAc. The solvent was removed to afford the product D.1.5 which was used without further purification.

### Step 4: Synthesis of 6-hydroxy-6,7-dihydro-1H-indeno[5,6-c]furan-1,3(5H)-dione (D.1.6)

The mixture of D.1.5 in Ac₂O was stirred at 120° C. for 6 hours. All volatiles were removed and the residue was chromatographed on silica gel to afford D.1.6.

### Step 5: Synthesis of 2-(2,6-dioxopiperidin-3-yl)-6-hydroxy-6,7-dihydrocyclopenta[f]isoindole-1,3(2H,5H)-dione (D.1.7)

To a solution of D.1.6 and 3-aminopiperidine-2,6-dione in toluene was added TEA (3 eq.). The mixture was stirred at reflux for 8 hours. All volatiles were removed and the residue was chromatographed on silica gel to afford D.1.7.

### Step 6: Synthesis of 2-(2,6-dioxopiperidin-3-yl)-5,7-dihydrocyclopenta[f]isoindole-1,3,6(2H)-trione (D.1)

To a solution of D.1.7 in DCM was added DMP (1.2 eq.). The reaction mixture was stirred at reflux for 4 hours. All volatiles were removed and the residue was chromatographed on silica gel to afford intermediate 2-(2,6-dioxopiperidin-3-yl)-5,7-dihydrocyclopenta[f]isoindole-1,3,6(2H)-trione (D.1). ESI-MS: 312.07.

### Example 8. Synthesis of 2-(2,6-dioxopiperidin-3-yl)-6,7,8,9-tetrahydroazepino[4,5-f]isoindole-1,3(2H,5H)-dione (E.1)

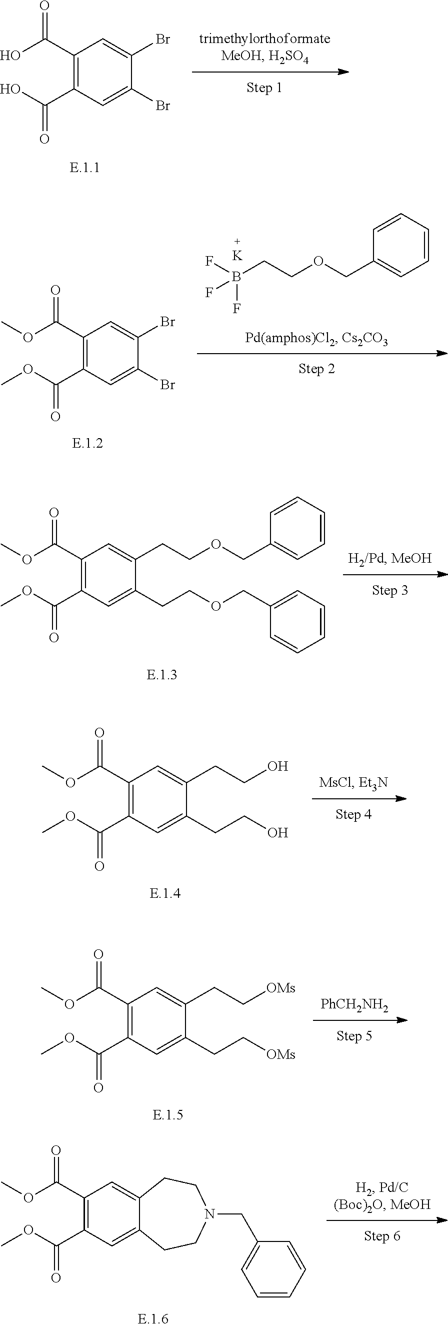

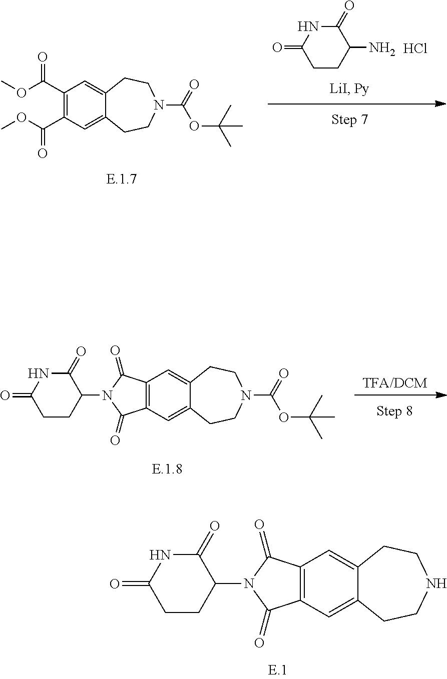

### Step 1: Synthesis of Dimethyl 4,5-dibromophthalate (E.1.2)

To a solution of 4,5-dibromophthalic acid (5 g) in MeOH (25 mL) and trimethyl orthoformate (25 mL) was added conc. H₂SO₄ (2.20 mL) at room temperature, and the reaction was refluxed overnight (about 12 h), solvent was removed under vacuum, EtOAc (100 mL) and sat. aq. NaHCO₃ (100 mL) was added. The products were extracted with EtOAc (50 mL×3), and the combined organic extracts were washed with brine, dried (Na₂SO₄), and concentrated in vacuo. The residue was used in the next step without further purification. ¹H NMR (400 MHz, CDCl₃) δ 3.91 (m, 6H), 7.97 (s, 2H).

### Step 2: Synthesis of dimethyl 4,5-bis(2-(benzyloxy)ethyl)phthalate (E.1.3)

Dimethyl 4,5-dibromophthalate (1.1 g, 3.13 mmol, 1.0 equiv), potassium (2-(benzyloxy) ethyl)trifluoroborate (1.66 g, 6.88 mmol, 2.2 equiv) and Cs₂CO₃ (4.58 g, 14.1 mmol, 4.5 equiv) was dissolved in toluene (25 mL)/water (12.5 mL). Pd(amphos)Cl₂ (325 mg, 0.46 mmol, 0.15 equiv) was added and the reaction mixture was stirred overnight (12 h) at 100° C. under N₂. After cooling to room temperature, the reaction mixture was extracted with EtOAc (20 mL×3), washed with brine, dried (Na₂SO₄), and concentrated in vacuo. The resulting crude product was purified by flash column chromatography on silica gel (petroleum ether/EtOAc=20:1 to 1:1) to give dimethyl 4,5-bis(2-(benzyloxy)ethyl)phthalate as colorless oil (910 mg, 62%). ¹H NMR (400 MHz, CDCl₃) δ 7.61 (s, 2H), 7.37-7.29 (m, 10H), 4.50 (s, 4H), 3.92 (s, 6H), 3.69 (t, J=7.6 Hz, 4H), 3.04 (t, J=7.6 Hz, 4H).

### Step 3: Synthesis of dimethyl 4,5-bis(2-hydroxyethyl)phthalate (E.1.4)

Dimethyl 4,5-bis(2-(benzyloxy)ethyl)phthalate (900 mg) was dissolved in MeOH. Pd/C (150 mg, 10%) was added and the reaction mixture was stirred overnight under H₂. The mixture was filtered and concentrated to give crude dimethyl 4,5-bis(2-hydroxyethyl)phthalate (510 mg, 93% yield) as a white solid. ¹H NMR (400 MHz, CDCl₃) δ 7.58 (s, 2H), 3.90 (t, J=6.4 Hz, 4H), 3.89 (s, 6H), 2.99 (t, J=6.6 Hz, 4H), 1.80 (brs, 2H).

### Step 4: Synthesis of dimethyl 4,5-bis(2-((methylsulfonyl)oxy)ethyl)phthalate (E.1.5)

Dimethyl 4,5-bis(2-hydroxyethyl)phthalate (282 mg, 1.0 mmol) and Et₃N (303 mg, 3.0 mmol, 3.0 equiv) was dissolved in DCM (8 mL) and MsCl (286 mg, 2.5 mmol, 2.5 equiv) was added at 0° C. in one portion, then stirred at rt for 45 mins. TLC showed the reaction was complete. DCM was added and the reaction mixture was washed with water, aq. NaHCO₃, brine, dried (Na₂SO₄), and concentrated to give dimethyl 4,5-bis(2-((methylsulfonyl)oxy)ethyl)phthalate (430 mg) that was used in the next step without further purification. ¹H NMR (400 MHz, CDCl₃) δ 7.61 (s, 2H), 4.30 (t, J=7.2 Hz, 4H), 3.91 (s, 6H), 3.18 (t, J=7.2 Hz, 4H), 2.96 (s, 6H).

### Step 5: Synthesis of dimethyl 3-benzyl-2,3,4,5-tetrahydro-1H-benzo[d]azepine-7,8-dicarboxylate (E.1.6)

Dimethyl 4,5-bis(2-((methylsulfonyl)oxy)ethyl)phthalate (430 mg) was dissolved in 1,2-dichloroethane (10 mL) and benzylamine (1.3 mL, 12 eqiv) was added. The reaction was stirred at 50° C. for 24 h. TLC shows the reaction was complete. DCM was added and the reaction mixture was washed with water, brine, and dried. The resulting crude product was purified by flash column chromatography on silica gel (petroleum ether/EtOAc=10:1 to 1:1) to give dimethyl 3-benzyl-2,3,4,5-tetrahydro-1H-benzo[d]azepine-7,8-dicarboxylate (196 mg). ¹H NMR (400 MHz, CDCl₃) δ 7.44 (s, 2H), 7.36-7.27 (m, 5H), 3.88 (s, 6H), 3.62 (s, 2H), 2.98-2.95 (m, 4H), 2.63-2.61 (m, 4H); LC-MS: [M+H]⁺=354.21

### Step 6: Synthesis of 3-(tert-butyl) 7,8-dimethyl 1,2,4,5-tetrahydro-3H-benzo[d]azepine-3,7,8-tricarboxylatedimethyl (E.1.7)

Dimethyl 3-benzyl-2,3,4,5-tetrahydro-1H-benzo[d]azepine-7,8-dicarboxylate (190 mg) was dissolved in MeOH, and (Boc)₂O (1.1 equiv) and Pd/C (80 mg, 10% by wt) were added. The reaction mixture was stirred overnight under H₂, and the mixture was filtered and concentrated to give crude 3-(tert-butyl) 7,8-dimethyl 1,2,4,5-tetrahydro-3H-benzo[d]azepine-3,7,8-tricarboxylatedimethyl. ¹H NMR (400 MHz, CDCl₃) δ 7.47 (s, 2H), 7.36-7.27 (m, 5H), 3.88 (s, 6H), 3.55-3.52 (m, 4H), 2.95-2.92 (m, 4H), 1.47 (s, 9H); LC-MS: [M+H]⁺=364.10

### Step 6: Synthesis of tert-butyl 2-(2,6-dioxopiperidin-3-yl)-1,3-dioxo-2,3,5,6,8,9-hexahydroazepino[4,5-f]isoindole-7(1H)-carboxylate (E.1.8)

3-(Tert-butyl) 7,8-dimethyl 1,2,4,5-tetrahydro-3H-benzo[d]azepine-3,7,8-tricarboxylate (73 mg, 0.2 mmol) and 3-aminopiperidine-2,6-dione hydrochloride (66 mg, 0.4 mmol, 2 equiv) were dissolved in pyridine (3 mL), and LiI (268 mg, 2 mmol, 10 equiv) was added. The reaction mixture was stirred at 130° C. for 15 h. LC-MS show the reaction was complete. The solvent was removed and purified by preparative HPLC to give E.1.8. LC-MS:[M+H]⁺=428.30.

### Step 7: Synthesis of 2-(2,6-dioxopiperidin-3-yl)-6,7,8,9-tetrahydroazepino[4,5-f]isoindole-1,3(2H,5H)-dione (E.1)

To a solution of tert-butyl 2-(2,6-dioxopiperidin-3-yl)-1,3-dioxo-2,3,5,6,8,9-hexahydroazepino[4,5-f]isoindole-7(1H)-carboxylate in DCM (2 mL) was added TFA (0.5 mL). The reaction mixture was stirred at rt for 1 h and the solvent was removed to give Cpd.

No. 855 as the TFA salt. ¹H NMR (400 MHz, DMSO-d₆) δ 11.11 (s, 1H), 9.01 (brs, 2H), 7.83 (s, 2H), 7.79 (s, 1H), 5.13 (dd, J=12.8, 5.4 Hz, 1H), 3.29-3.23 (m, 8H), 2.93-2.85 (m, 1H), 2.63-2.51 (m, 2H), 2.09-2.03 (m, 1H); LC-MS: [M+H]⁺=328.21.

### Example 9. Synthesis of 2-(2,6-dioxopiperidin-3-yl)-6,7,8,9-tetrahydroazepino[3,4-f]isoindole-1,3(2H,5H)-dione (F.1)

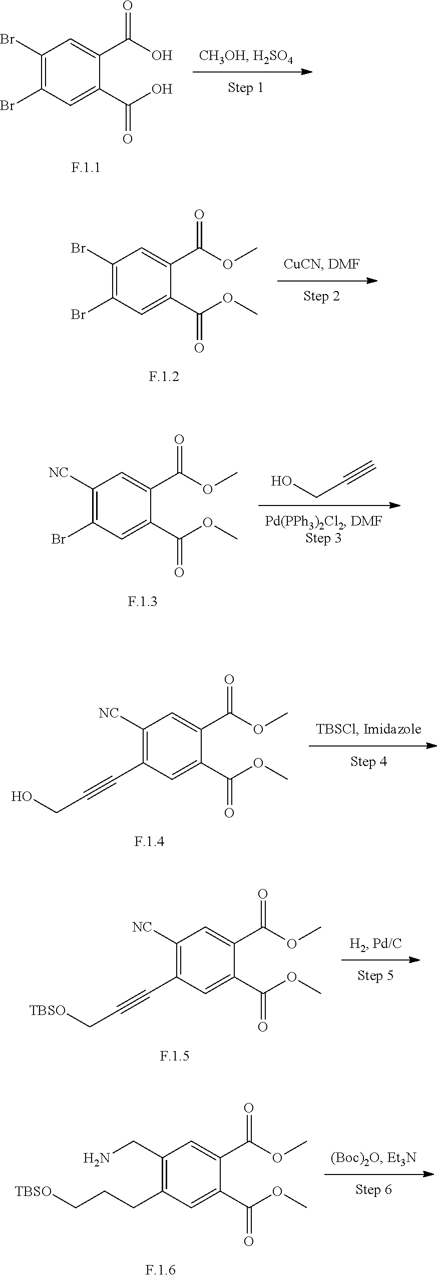

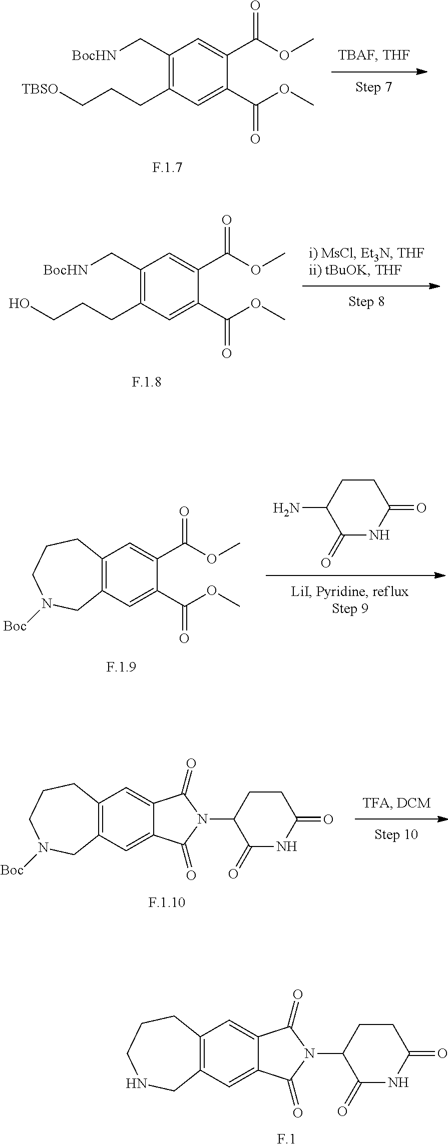 [SMILES: COC(=O)c1cc(CCCO[Si](C)(C)C(C)(C)C)c(CNC(=O)OC(C)(C)C)cc1C(=O)OC ; COC(=O)c1cc(CCCO)c(CNC(=O)OC(C)(C)C)cc1C(=O)OC ; COC(=O)c1cc2c(cc1C(=O)OC)CN(C(=O)OC(C)(C)C)CCC2 ; NC1CCC(=O)NC1=O ; CC(C)(C)OC(=O)N1CCCc2cc3c(cc2C1)C(=O)N(C1CCC(=O)NC1=O)C3=O ; O=C1CCC(N2C(=O)c3cc4c(cc3C2=O)CNCCC4)C(=O)N1]

### Step 1: Synthesis of Dimethyl 4,5-dibromophthalate (F.1.2)

To a solution of 4,5-dibromophthalic acid (5 g) and trimethyl orthoformate (25 mL) in MeOH (25 mL) was added conc. H₂SO₄ (2 mL) at room temperature, and the reaction was refluxed overnight. The solvent was removed under vacuum, and EtOAc (100 mL) and sat. aq. NaHCO₃ (20 mL) were added. The reaction mixture was extracted with EtOAc (50 mL×3), and the combined organic extracts were washed with brine, dried (Na₂SO₄), and concentrated in vacuo. The residue was used in the next step without further purification. ¹H NMR (400 MHz, CDCl3) δ 3.91 (m, 6H), 7.97 (s, 2H).

### Step 2: Synthesis of Dimethyl 4-bromo-5-cyanophthalate (F.1.3)

Dimethyl 4,5-dibromophthalate (1.5 g, 4.28 mmol) and copper(I) cyanide (500 mg, 5.56 mmol) were dissolved with 15 ml of anhydrous DMF and stirred at 100° C. overnight. The reaction mixture was extracted with ethyl ether three times and the organic phase was washed with cold water and brine to remove the excess DMF. Removal of the solvent followed by purification by flash chromatography on silica gel (ethyl acetate-hexane) gave dimethyl 4-bromo-5-cyanophthalate (F.1.3) in 60% yield. LC-MS: [M+H]⁺=297.96.

### Step 3: Synthesis of Dimethyl 4-cyano-5-(3-hydroxyprop-1-yn-1-yl)phthalate (F.1.4)

Compound F.1.3 (1.1 g, 3.71 mmol, 1.0 eq), Pd(PPh₃)₂Cl₂ (263 mg, 0.371 mmol, 0.1 eq), CuI (140 mg, 0.742 nmol. 0.2 eq.), and propargyl alcohol (0.312 g, 5.57 mmol, 1.5 eq.) were dissolved with 15 mL of dry DMF, and the reaction vessel was purged with nitrogen balloon three times. Et₃N (3 mL) was added and the reaction mixture was heated to 80° C. for 2 h. The reaction mixture was extracted with ethyl ether three times and washed with cold water and brine to remove the excess DMF. Removal of the solvent followed by purification by flash chromatography on silica gel (ethyl acetate-hexane) gave dimethyl 4-cyano-5-(3-hydroxyprop-1-yn-1-yl)phthalate (F.1.4) in 70% yield. LC-MS: [M+H]⁺=274.06.

### Step 4: Synthesis of Dimethyl 4-(3-((tert-butyldimethylsilyl)oxy)prop-1-yn-1-yl)-5-cyanophthalate (F.1.5)

To a solution of compound F.1.4 (500 mg, 1.83 mmol) and imidazole (373 mg, 5.49 mmol), in dry DCM (10 mL) was added TBSCl (412 mg, 2.74 mmol) under N₂ at room temperature. The reaction mixture was stirred at room temperature for 1 h. The mixture was quenched with H₂O and extracted with DCM. The organic layers were separated and washed with H₂O, brine, dried (MgSO₄), and then purification by flash chromatography on silica gel (ethyl acetate-hexane) to give compound F.1.5 as 90% yield. LC-MS: [M+H]⁺=388.15.

### Step 5: Synthesis of Dimethyl 4-(aminomethyl)-5-(3-((tert-butyldimethylsilyl)oxy)propyl)phthalate (F.1.6)

Compound 5 (900 mg) was dissolved in MeOH and Pd/C (90 mg, 10% by wt) was added. The reaction mixture was stirred overnight under H₂. The reaction mixture was filtered and concentrated to give crude dimethyl 4-(aminomethyl)-5-(3-((tert-butyldimethylsilyl)oxy)propyl)phthalate (F.1.6). LC-MS: [M+H]⁺=396.21.

### Step 6: Synthesis of Dimethyl 4-(((tert-butoxycarbonyl)amino)methyl)-5-(3-((tert-butyldimethylsilyl)oxy)propyl)phthalate (F.1.7)

Crude compound 6 was dissolved in dry DCM, and Boc₂O (1.1 eq.) and Et₃N (3.0 eq.) were added. The reaction mixture was stirred at room temperature for 2 h. The reaction mixture was concentrated under reduced pressure and purified by flash chromatography on silica gel (ethyl acetate-hexane) to give compound F.1.7 in 60% yield. LC-MS: [M+H]⁺=496.27.

### Step 7: Synthesis of dimethyl 4-(((tert-butoxycarbonyl)amino)methyl)-5-(3-hydroxypropyl)phthalate (F.1.8)

Compound 7 (163 mg, 0.33 mmol) was suspended in dry THE (5 mL) and cooled in an ice bath. TBAF (1M in THF, 0.66 mL, 0.66 mmol) was added and the reaction mixture was allowed to warm to room temperature and stir for 3 h. The mixture was concentrated in vacuo, diluted with EtOAc, and washed with sat aq. NH₄Cl. The organic layer was concentrated to provide the crude product which was purified by flash chromatography on silica gel (ethyl acetate:hexane=1:1) to give compound F.1.8 in 80% yield. LC-MS: [M+H]⁺=382.18.

### Step 8: Synthesis of 2-(tert-butyl) 7,8-dimethyl 1,3,4,5-tetrahydro-2H-benzo[c]azepine-2,7,8-tricarboxylate (F.1.9)

Compound F.1.8 (200 mg, 0.52 mmol) and Et₃N (131 mg, 1.3 mmol, 2.5 equiv) were dissolved in dry THE (4 mL), and MsCl (89 mg, 0.78 mmol, 1.5 equiv) was added at 0° C. in one portion. The reaction mixture was stirred at rt for 45 min. TLC showed the reaction was complete. The reaction mixture was treated with t-BuOK (1.5 ml 1 (M) THF, 3 equiv) and stirred for an additional 2 h. The reaction mixture was quenched by adding water and extracted with EtOAc. The organic layer was concentrated to provide the crude product which was purified by flash chromatography on silica gel (ethyl acetate:hexane=1:1) to give compound F.1.9 in 60% yield. LC-MS: [M+H]⁺=364.17.

### Step 9: Synthesis of tert-butyl 2-(2,6-dioxopiperidin-3-yl)-1,3-dioxo-2,3,5,7,8,9-hexahydroazepino[3,4-f]isoindole-6(1H)-carboxylate (F.1.10)

Compound F.1.9 (70 mg, 0.2 mmol) and 3-aminopiperidine-2,6-dione hydrochloride (66 mg, 0.4 mmol, 2 equiv) were dissolved in pyridine (3 mL) and LiI (268 mg, 2 mmol, 10 equiv) was added. The reaction mixture was stirred at 130° C. for 15 h. LC-MS show the reaction was >85% complete. The solvent was removed and the crude product purified by preparative HPLC to give compound F.1.10. LC-MS: [M+H]⁺=428.17.

### Step 10: Synthesis 2-(2,6-dioxopiperidin-3-yl)-6,7,8,9-tetrahydroazepino[3,4-f]isoindole-1,3(2H,5H)-dione (F.1)

Compound F.1.10 (102.1 mg, 0.28 mmol) was added to 1 mL HCl (4M in 1,4-dioxane), and the mixture reaction mixture was stirred at room temperature for 2 h. The 1,4-dioxane was removed under reduced pressure to give F.1 as the HCl salt. LC-MS: [M+H]⁺=328.17. ¹H NMR (400 MHz, MeOH-d₄) δ 7.88 (s, 1H), 7.79 (s, 1H), 5.11 (dd, J=12.6, 5.4 Hz, 1H), 4.54 (s, 2H), 3.54-3.47 (m, 2H), 3.29-3.19 (m, 3H), 2.90-2.62 (m, 3H), 2.16-2.06 (m, 1H), 2.06-1.95 (m, 2H).

### Example 10. Synthesis of 3-(4-methoxy-1-oxo-3,5,6,7-tetrahydropyrrolo[3,4-f]isoindol-2(1H)-yl)piperidine-2,6-dione (G.1)

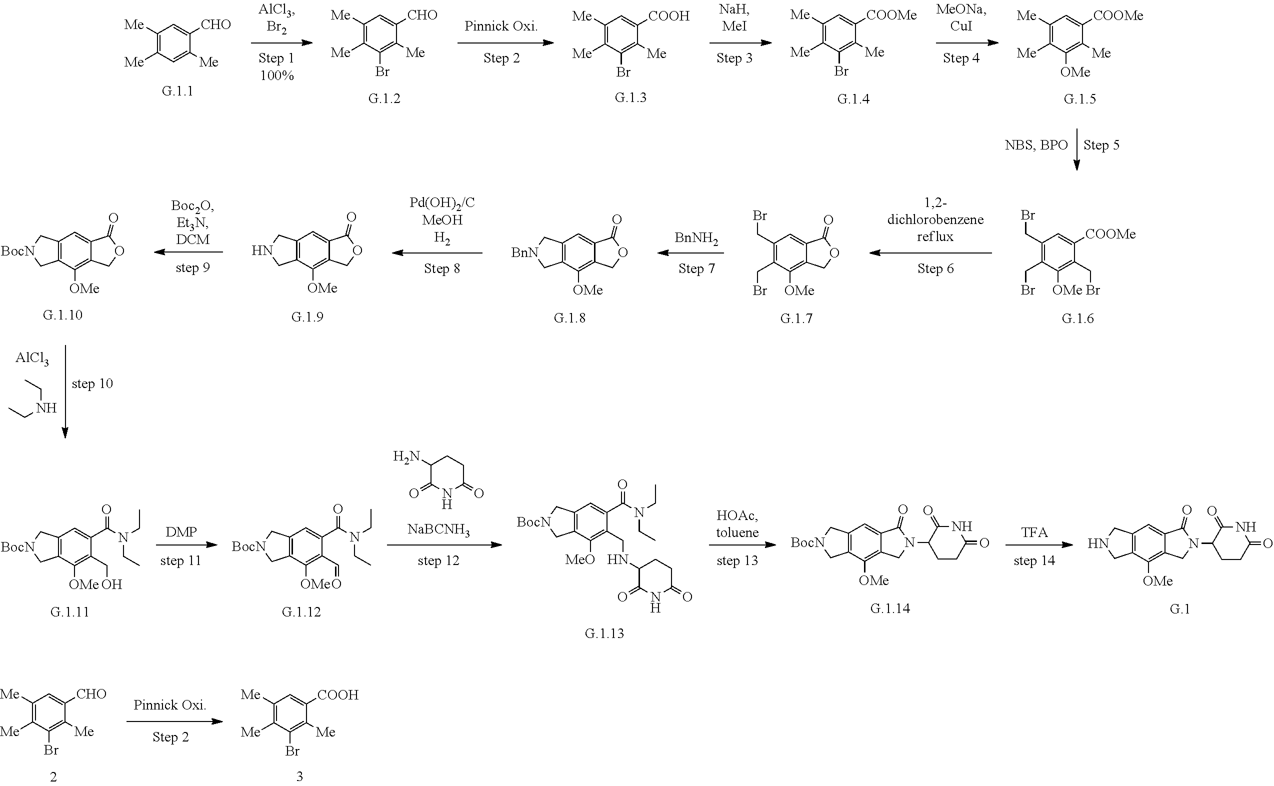

To a solution of 2 (1 equiv) in t-BuOH/H2O=3:1 (c2=0.25 mol/L). NaH2PO4 (5 equiv) and Isoamylene (10 equiv) was added into the flask at room temperature. Then NaClO (3 equiv) was added into the flask. Quenched with saturated NaHSO3, The organic phase was separated. EA was added to the mixture, the resulting mixture was washed by brine. The combined organic phase was dried by MgSO4. Filtered and concentrated in vacuum. The residue was used for next step without further purification.

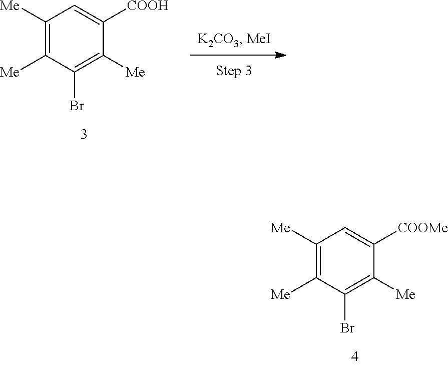 [SMILES: Cc1cc(C(=O)O)c(C)c(Br)c1C ; COC(=O)c1cc(C)c(C)c(Br)c1C]

To a solution of 3 (1 equiv) in acetone (c3=0.5 mol/L). K₂CO₃ (3 equiv) and Mel (1.2 equiv) was added into the flask at room temperature. Quenched with saturated NaHCO3. EA was added to the mixture, The organic phase was separated. the resulting mixture was washed by brine. The combined organic phase was dried by MgSO4. Filtered and concentrated in vacuum. The residue was purified by silica gel chromatography, the combination yield of two steps is 80%.

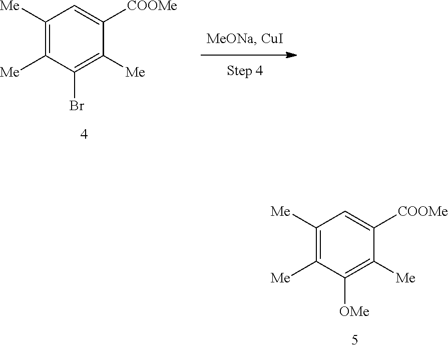 [SMILES: COC(=O)c1cc(C)c(C)c(Br)c1C ; COC(=O)c1cc(C)c(C)c(OC)c1C]

To a solution of 4 (1 equiv) in DMF (C₄=0.25 mol/L). MeONa (25 wt % in methanol, 2 equiv) and CuI (1 equiv), EA (1 equiv) was added into the flask. Heat to 100° C. for 6 h. Quenched with 3M HCl. EA was added to the mixture, The organic phase was separated. the resulting mixture was washed by brine. The combined organic phase was dried by MgSO₄. Filtered and concentrated in vacuum. The residue was purified by silica gel chromatography, yield 83%.

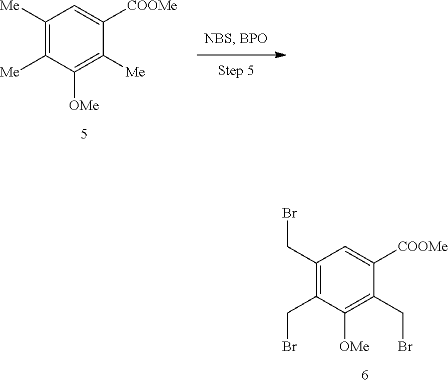 [SMILES: COC(=O)c1cc(C)c(C)c(OC)c1C ; COC(=O)c1cc(CBr)c(CBr)c(OC)c1CBr]

5 (1.0 equiv.) and N-bromosuccinimide (3.2 equiv.) were dissolved in 2-dichloroethane (c₅=0.5 mol/L), the reaction mixture was heated to 80° C. and benzoyl peroxide (0.02 equiv.) was added in one portion. Heating continued for 8 h. TLC (PE:EA=5:1, Rf=0.4) checked that 5 was consumed. The reaction mixture was cooled to ambient temperature, then filtered and the filtrates were concentrated. The residue was purified by silica gel chromatography to give 6 at 50% yield.

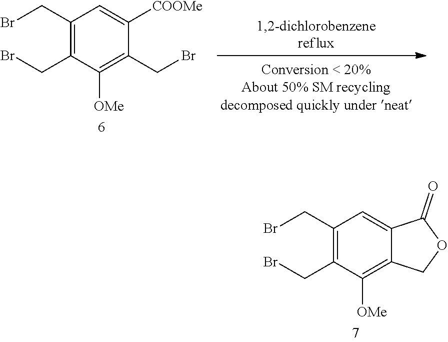 [SMILES: COC(=O)c1cc(CBr)c(CBr)c(OC)c1CBr ; COc1c(CBr)c(CBr)cc2c1COC2=O]

6 (1.0 equiv.) was dissolved in 1,2-dichlorobenzene (c₆=0.05 mol/L), the reaction mixture was refluxed for 24 hours. TLC (PE:EA=1:1, Rf=0.2) checked that 7 was appeared. The reaction mixture was cooled to ambient temperature, then was purified by silica gel chromatography straightly to give 7 at 10% yield, about 50% SM can be recycled.

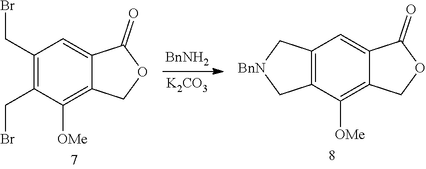

To a solution of 7 (1 equiv) in MeCN (c7=0.2 mol/L). K₂CO₃ (3 equiv) and BnNH₂ (1 equiv) was added into the flask at room temperature in 5 portions. Reaction was quenched with saturated NaHCO3. EA was added to the mixture, The organic phase was separated. the resulting mixture was washed by brine. The combined organic phase was dried by MgSO₄. Filtered and concentrated in vacuum. The residue was purified by silica gel chromatography to give 8 at 48% yield.

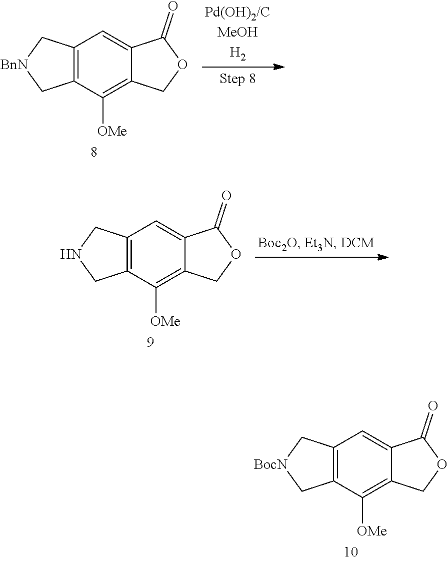

To a solution of 8 (1 equiv) in MeOH (c₈=0.05 mol/L). Pd(OH)₂/C (¼ of 8's weight) was added into the flask. Hydrogenation with H₂ balloon for 3 h. Reaction was detected by UPLM-MS. Pd(OH)₂/C was filtered by cilite. concentrated in vacuum to get 9 without more purification. 9 was dissolved in DCM (c₉=0.1 mol/L). Et₃N (1.5 equiv) and Boc₂O (1.2 equiv) were added into the flask. Reaction was detected by UPLM-MS. Concentrated and purified by silica gel chromatography to give 10 at 58% yield.

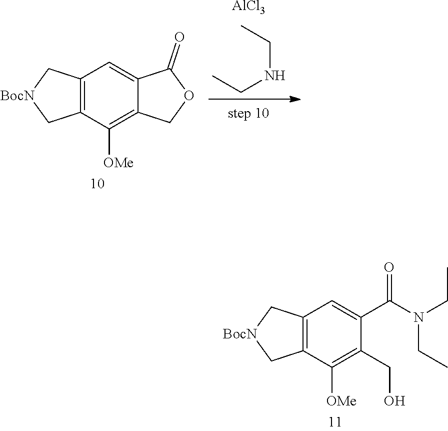

To a suspension of aluminium trichloride (1.3 equiv.) in DCM (c_{AlC13}=0.5 mol/L), diethylamine (2.5 equiv.) was added at 0° C. and the mixture was stirred for additional 30 min. A solution of 10 (1.0 eq.) in DCM (c₁₀=1 mol/L), was added and the resulting mixture was stirred at 25° C. for 1 h. UPLM-MS(11: M+H:379,M+H−H₂O:361) checked that 10 was consumed. The reaction mixture was poured into 300 mL saturated aqueous NH₄Cl. The organic layers were combined and washed with 200 mL saturated aqueous NH₄Cl, dried over anhydrous MgSO₄, and concentrated in vacuum. The obtained residue was purified by silica gel chromatography to give 11 (85% yield).

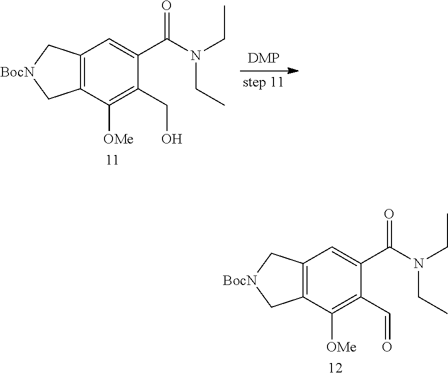

To a solution of 11 (1 equiv) in DCM (c₁=0.1 mol/L). DMP (1.1 equiv) was added into the flask at room temperature. Reaction was detected by UPLM-MS. Quenched with saturated NaHCO₃. EA was added to the mixture, The organic phase was separated. the resulting mixture was washed by brine. The combined organic phase was dried by MgSO₄. Filtered and concentrated in vacuum. The residue was purified by silica gel chromatography to give 12 (90%).

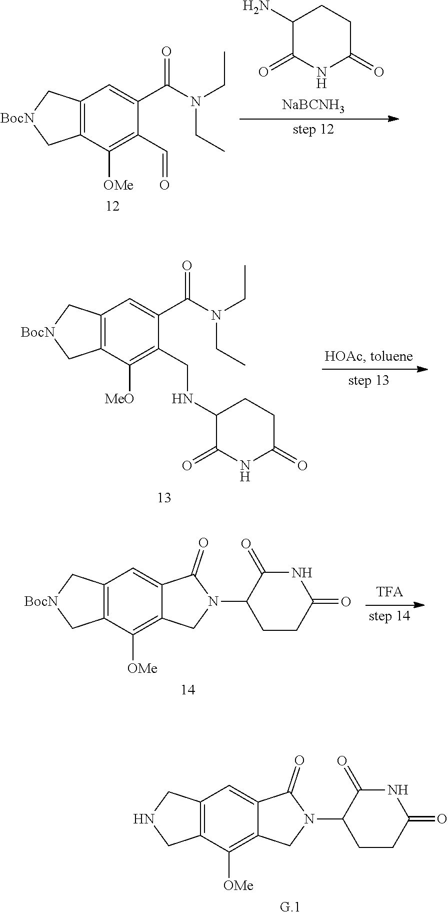

To a solution of 12 (1 equiv) in MeOH (c₁=0.2 mol/L). NaOAc (1.0 equiv) and (S)-3-Amino-piperidine-2,6-dione hydrochloride NaOAc (1.0 equiv), NaCNBH₃ (1.0 equiv) was added into the flask at room temperature. Reaction was detected by UPLM-MS (about 3 hours). Remove solvent under vacuum. The residue was dissolved in toluene. HOAc(15 equiv) was added into flask. The reaction was heated at 110° C. and stirred for 12 hour. Reaction was detected by UPLM-MS, all 13 is change into 14. 14 was purified by HPLC(TFA condition). 1.0 equiv TFA was added and concentrated 14 to get de-Boc G.1 (63% yield in three steps).

### Example 11. Synthesis of 2′-(2,6-dioxopiperidin-3-yl)-5′,7′-dihydro-1′H-spiro[azetidine-3,6′-cyclopenta[f]isoindole]-1′,3′(2′H)-dione (H)

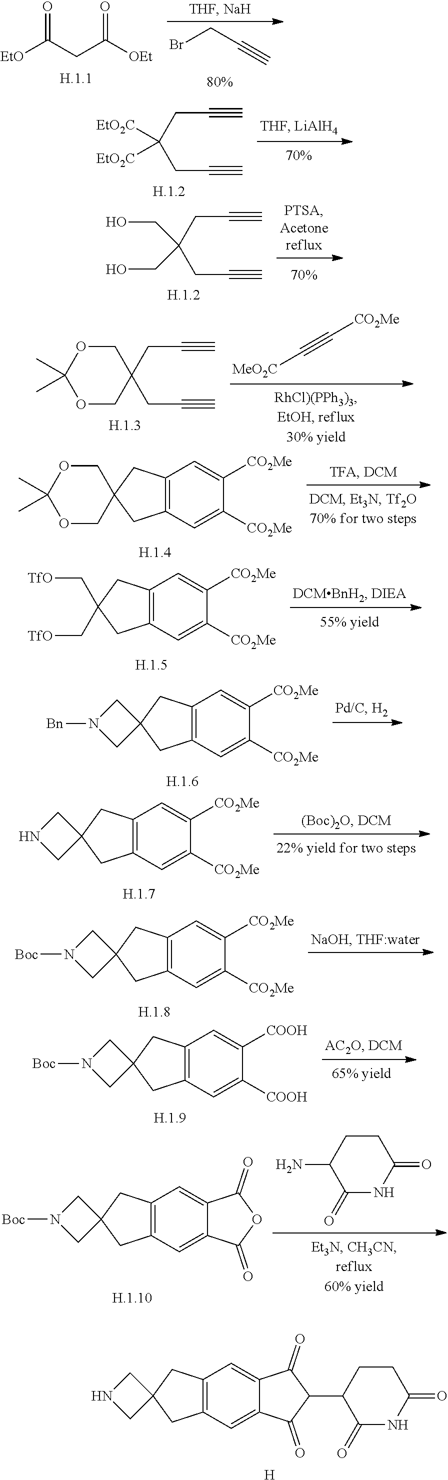

¹H NMR of compound H (400 MHz, Methanol-d₄) δ 7.76 (s, 2H), 5.14 (dd, J=12.6, 5.5 Hz, 1H), 4.15 (s, 4H), 3.47 (s, 4H), 2.97-2.62 (m, 3H), 2.20-2.09 (m, 1H).

### Example 12. synthesis of 2′-(2,6-dioxopiperidin-3-yl)-5′,7′-dihydro-1′H-spiro[azetidine-3,6′-cyclopenta[f]isoindole]-1′,3′(2′H)-dione (I)

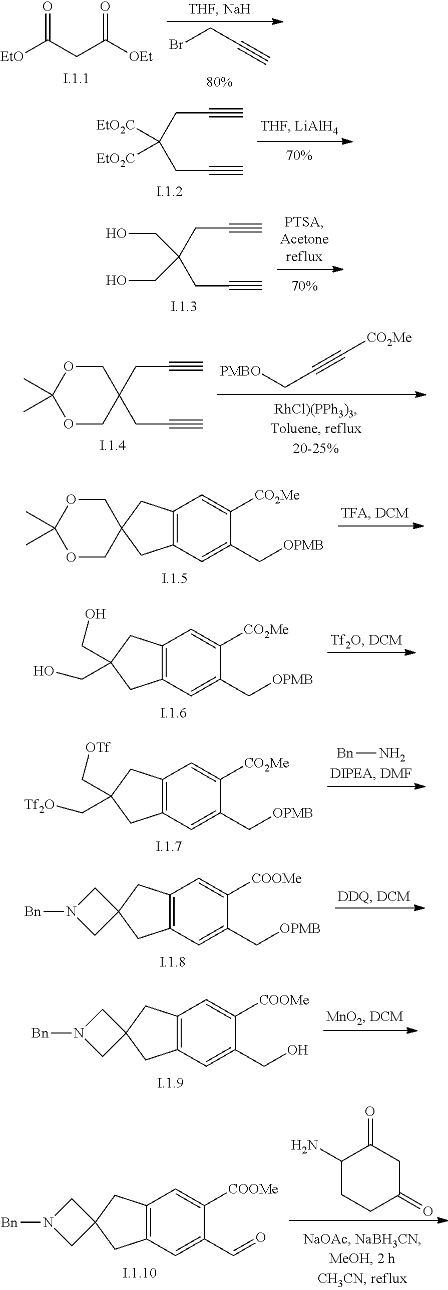

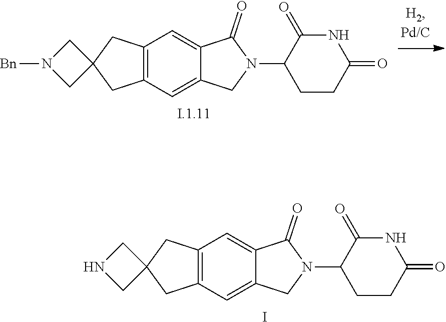

¹H NMR of compound 1 (400 MHz, Methanol-d₄) δ 7.65 (s, 1H), 7.44 (s, 1H), 5.15 (dd, J=13.4, 5.2 Hz, 1H), 4.45 (d, J=7.3 Hz, 2H), 3.91 (d, J=3.8 Hz, 4), 3.26 (d, J=5.9 Hz, 4H), 2.92 (ddd, J=17.6, 13.4, 5.3 Hz, 1H), 2.80 (ddd, J=17.7, 4.7, 2.5 Hz, 1H), 2.50 (qd, J=13.2, 4.7 Hz, 1H), 2.18 (dtd, J=12.9, 5.3, 2.5 Hz, 1H).

### Example 13. Synthesis of 2-(azetidin-3-ylmethyl)-6-(2,6-dioxopiperidin-3-yl)-2,3,6,7-tetrahydropyrrolo[3,4-f]isoindole-1,5-dione is shown in Scheme 5. (S)-2-(Azetidin-3-ylmethyl)-6-(2,6-dioxopiperidin-3-yl)-2,3,6,7-tetrahydropyrrolo[3,4-f]isoindole-1,5-dione (J)

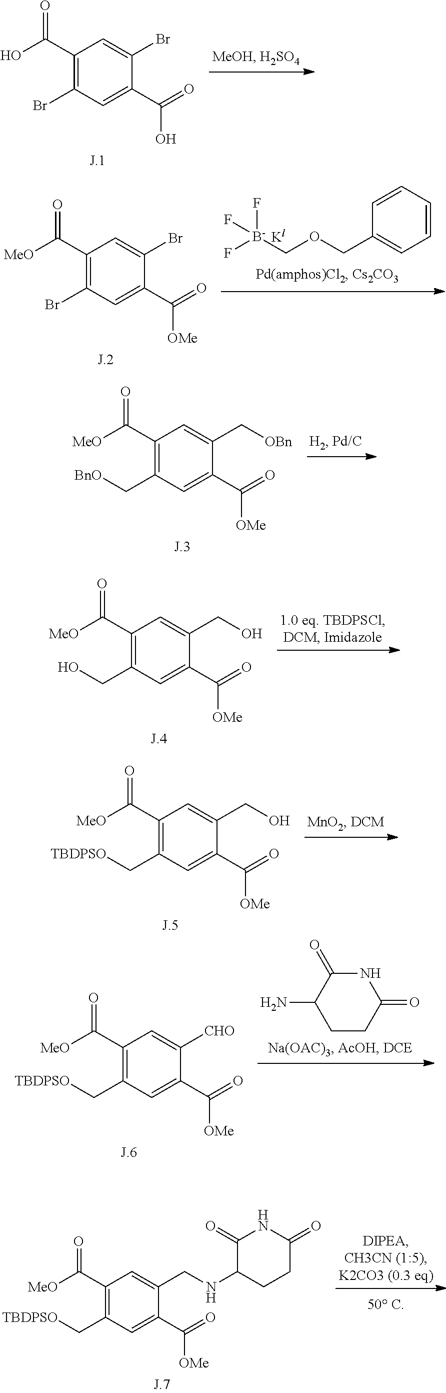

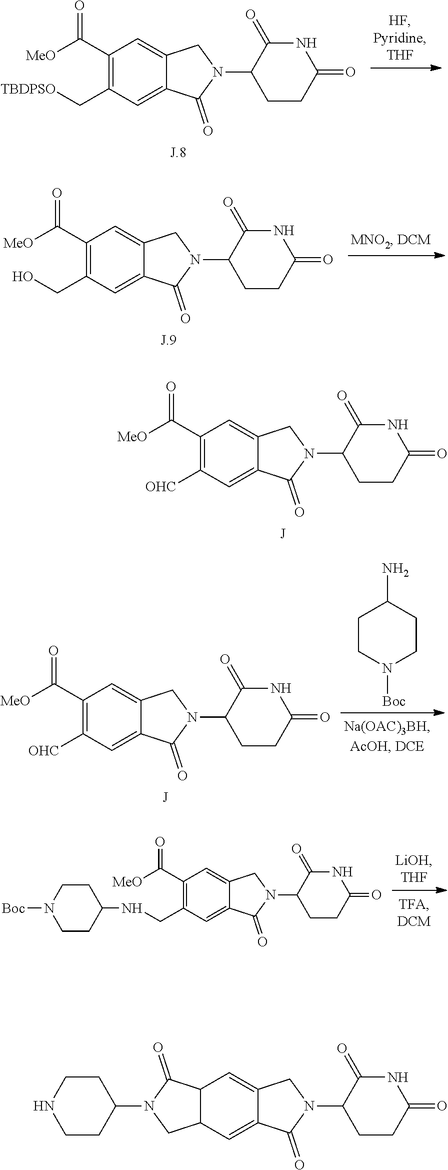

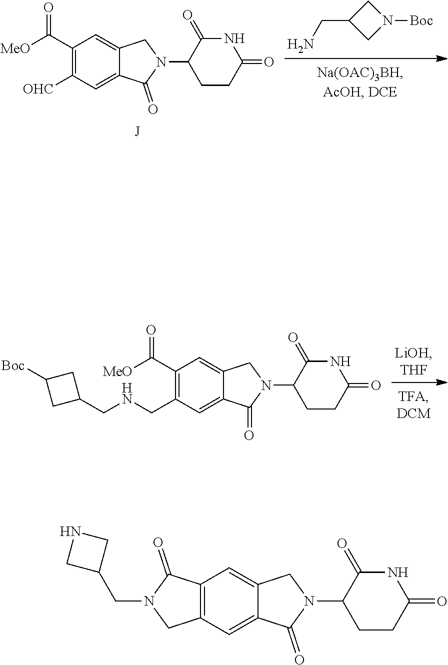 [SMILES: COC(=O)c1cc2c(cc1C=O)C(=O)N(C1CCC(=O)NC1=O)C2 ; CC(C)(C)OC(=O)N1CC(CN)C1 ; COC(=O)c1cc2c(cc1CNCC1CC(C(=O)OC(C)(C)C)C1)C(=O)N(C1CCC(=O)NC1=O)C2 ; O=C1CCC(N2Cc3cc4c(cc3C2=O)CN(CC2CNC2)C4=O)C(=O)N1]

¹H NMR of compound J (400 MHz, CDCl₃) δ 10.60 (s, 1H), 8.46 (s, 1H), 8.08 (s, 1H), 7.97 (s, 1H), 5.27 (dd, J=13.3, 5.2 Hz, 1H), 4.68 (d, J=16.8 Hz, 1H), 4.47 (d, J=16.9 Hz, 1H), 4.05 (s, 3H), 3.04-2.78 (m, 2H), 2.50-2.36 (m, 1H), 2.36-2.24 (m, 1H).

### Example 14. Synthesis of 2-(2,6-dioxopiperidin-3-yl)-6-(2-(9-(4-((1R,2S)-6-hydroxy-2-phenyl-1,2,3,4-tetrahydronaphthalen-1-yl)phenyl)-3,9-diazaspiro[5.5]undecan-3-yl)-2-oxoethyl)-6,7-dihydropyrrolo[3,4-f]isoindole-1,3(2H,5H)-dione (E149)

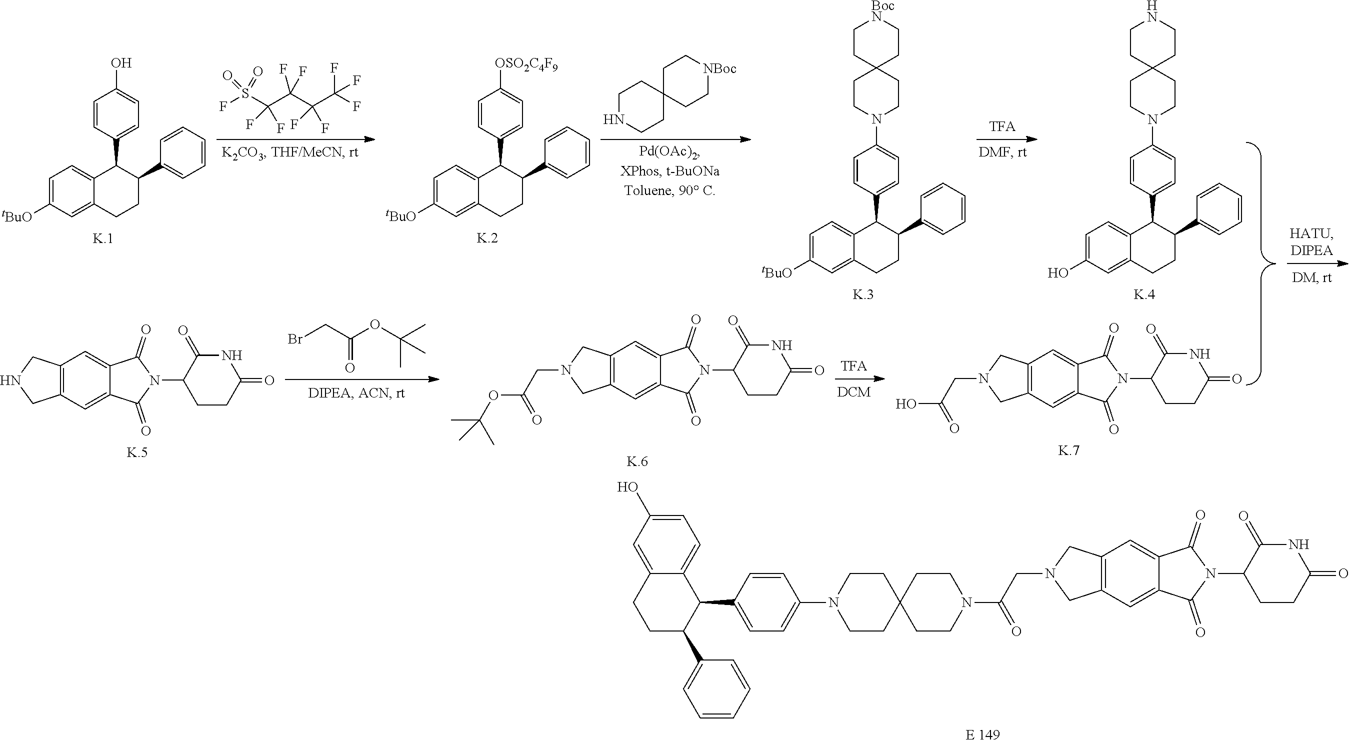

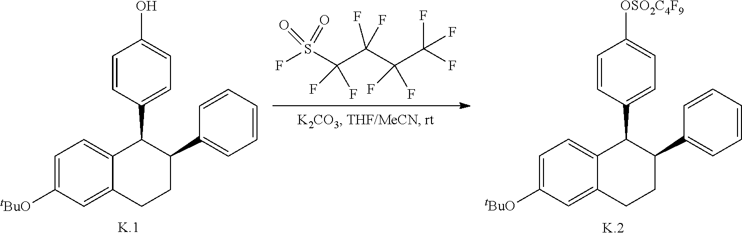

To a solution of K.1 (2.0 g, 1 eq) and 1,1,2,2,3,3,4,4,4-nonafluorobutane-1-sulfonyl fluoride (3.24 g, 2.0 eq) in tetrahydrofuran (10 mL) and acetonitrile (10 mL) was added potassium carbonate (2.6 g, 3.5 eq). The reaction mixture was stirred at rt for 16 hours. TLC (petroleum ether:ethyl acetate=10:1) indicated the starting material was consumed completely and one new spot formed. The reaction mixture was concentrated under reduced pressure. The residue was purified by silica gel flash chromatography (petroleum ether:ethyl acetate=100:0 to 95:5). The desired compound K.2 (3.5 g, >99% yield) was obtained as a colorless oil. 1H NMR (400 MHz, CDCl3) δ 7.21-7.11 (m, 3H), 6.94-6.86 (m, 3H), 6.84-6.73 (m, 4H), 6.46 (d, J=8.8 Hz, 2H), 4.33 (d, J=5.2 Hz, 1H), 3.50-3.40 (m, 1H), 3.16-2.95 (m, 2H), 2.20-2.02 (m, 1H), 1.91-1.79 (m, 1H), 1.38 (s, 9H).

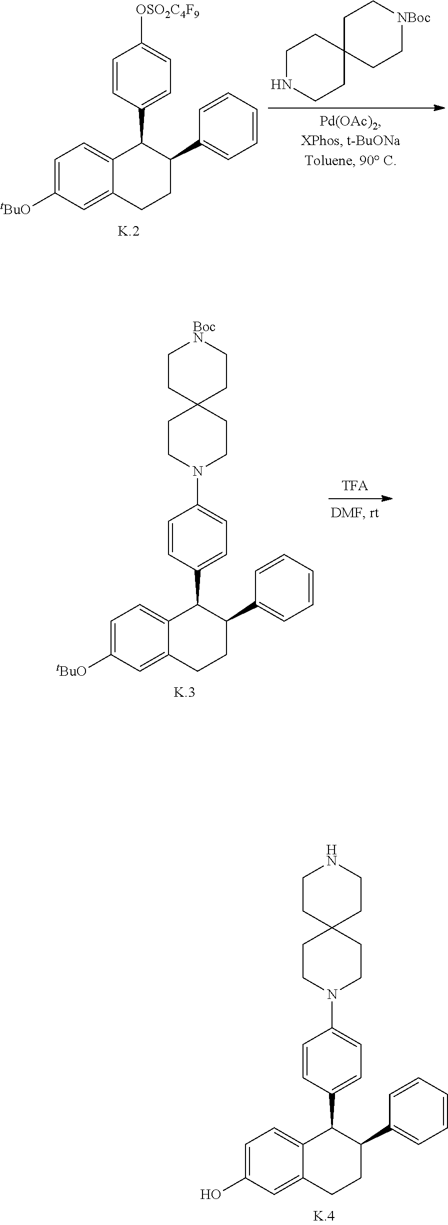

A mixture of K.2 (500 mg, 1.0 eq), tert-butyl 3,9-diazaspiro[5.5]undecane-3-carboxylate (389 mg, 2.0 eq), Pd(OAc)₂ (73 mg, 0.15 eq), XPhos (73 mg, 0.2 eq) and t-BuONa (257 mg, 3.5 eq) in tolune (10 mL) was degassed and purged with N₂ 3 times, and then the mixture was stirred at 90° C. for 16 h under N₂ atmosphere. LC-MS showed one main peak with desired MS was detected. TLC (PE:EA=10:1) indicated the starting material was consumed completely, and a new spot formed. The mixture was cooled, diluted with EA, filtered through Celite, the filter cake was washed with EA. The filtrate was concentrated. The residue was purified by silica gel flash chromatography (PE:EA=100:0 to 85:15). The desired product K.3 (300 mg, 65% yield) was obtained as a colorless oil. LCMS (ESI) m/z: 609.34 [M+1].

To a solution of K.3 (300 mg) in DCM (5 mL) was added TFA (2.5 mL). The reaction mixture was stirred overnight, then concentrated under reduced pressure. The residue was lyophilized, and compound K.4 (325 mg) was obtained as a white solid.

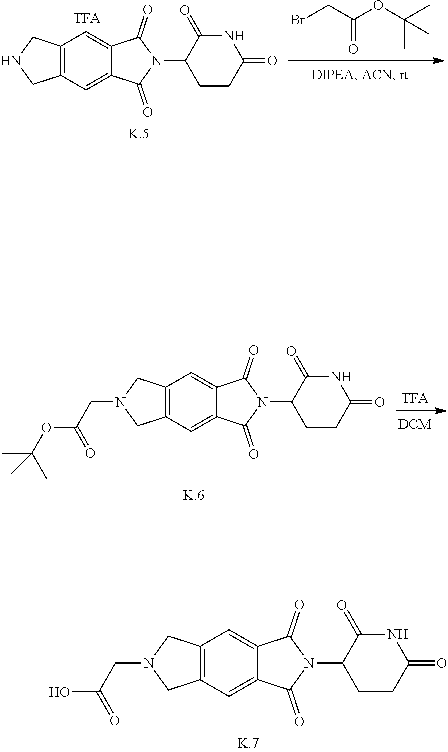

To a solution of K.5 (500 mg, 1.0 eq) in MeCN (25 mL) was added DIPEA (2.11 mL, 10.0 eq) and BrCH₂COO^{t}Bu (187.6 μL, 1.05 eq), and then the mixture was stirred at rt for 12 h. LC-MS showed one main peak with desired MS was detected. TLC (DCM:MeOH=20:1) indicated a new spot formed. Then the reaction mixture was concentrated under reduced pressure, and the residue was purified by silica gel flash chromatography (DCM:MeOH=100:0 to 95:5). The desired product K.6 was obtained as a light yellow powder. LCMS (ESI) m/z: 414.05 [M+1].

Compound K.6 was dissolved in DCM (5 mL), and then TFA (5 mL) was added. 2 h later, LC-MS showed starting material K.6 was consumed completely, and a new peak with desired MS was detected. The reaction mixture was concentrated under reduced pressure, and the resulted residue was lyophilized to obtained product 7 (440 mg) as a gray solid.

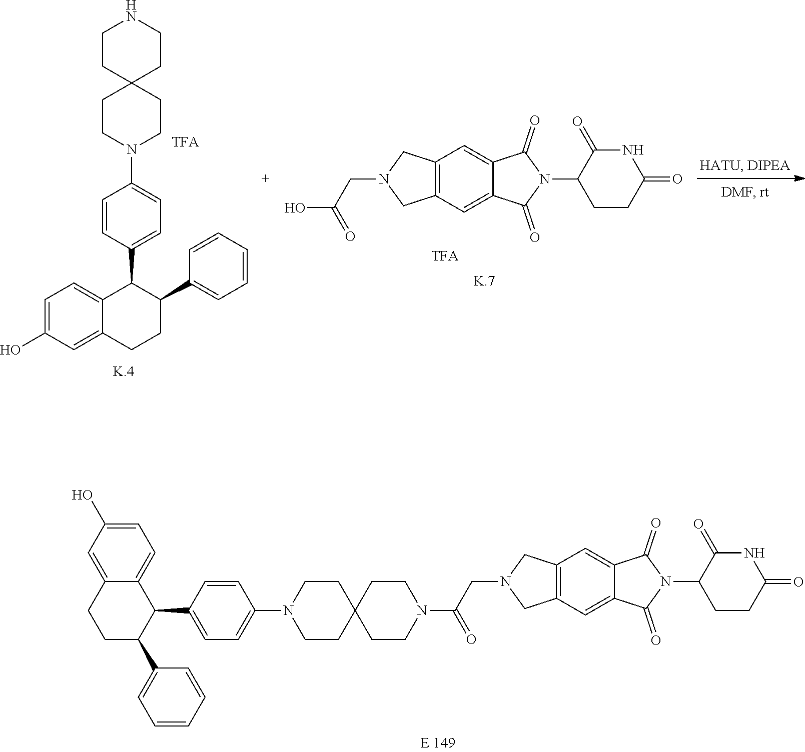 [SMILES: Oc1ccc2c(c1)CC[C@H](c1ccccc1)[C@@H]2c1ccc(N2CCC3(CCNCC3)CC2)cc1 ; O=C(O)CN1Cc2cc3c(cc2C1)C(=O)N(C1CCC(=O)NC1=O)C3=O ; O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1]

To a solution of 7 (166 mg, 1.0 eq) and HATU (134 mg, 1.0 eq) in DMF (6 mL) was added DIPEA (369 μL, 6.0 eq). 5 min later, 4 (200 mg, 1.0 eq) was added, and the reaction was stirred for 10 minutes. Then quenched with water 3 mL, and immediately purified by pre-HPLC (ACN/H₂O (25% to 100%) in 75 min, 60 mL/min), the desired product came out when ACN/H₂O=35%. The title compound 8 (ERD-1173, 186 mg, 53% yield) was obtained as a white solid after concentrated and lyophilized. LCMS (ESI) m/z: 792.22 [M+1].

### Example 15. Synthesis of 6-(2-(9-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-3,9-diazaspiro[5.5]undecan-3-yl)-2-oxoethyl)-2-(2,6-dioxopiperidin-3-yl)-6,7-dihydropyrrolo[3,4-f]isoindole-1,3(2H,5H)-dione (E 368)

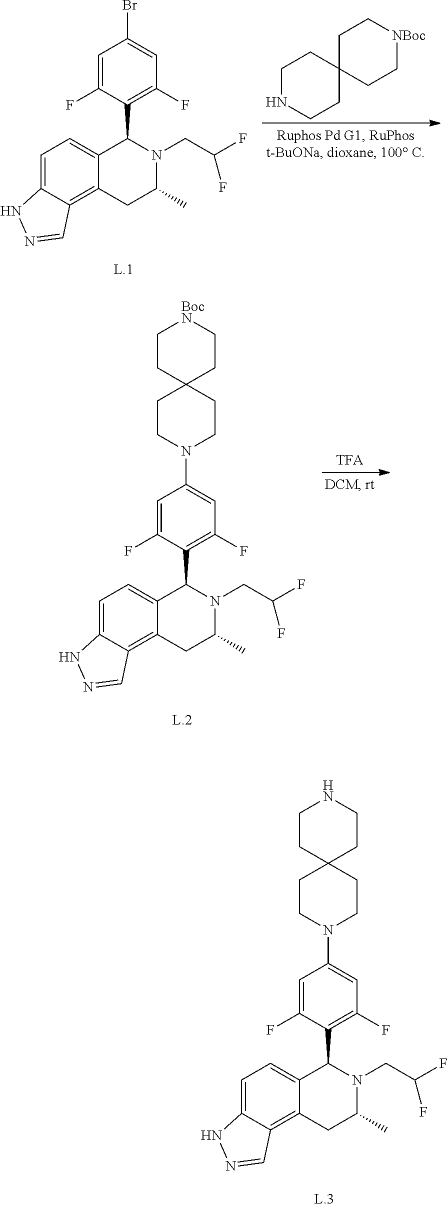 [SMILES: C[C@@H]1Cc2c(ccc3[nH]ncc23)[C@@H](c2c(F)cc(Br)cc2F)N1CC(F)F ; CC(C)(C)OC(=O)N1CCC2(CCNCC2)CC1 ; C[C@@H]1Cc2c(ccc3[nH]ncc23)[C@@H](c2c(F)cc(N3CCC4(CCN(C(=O)OC(C)(C)C)CC4)CC3)cc2F)N1CC(F)F ; C[C@@H]1Cc2c(ccc3[nH]ncc23)[C@@H](c2c(F)cc(N3CCC4(CCNCC4)CC3)cc2F)N1CC(F)F]

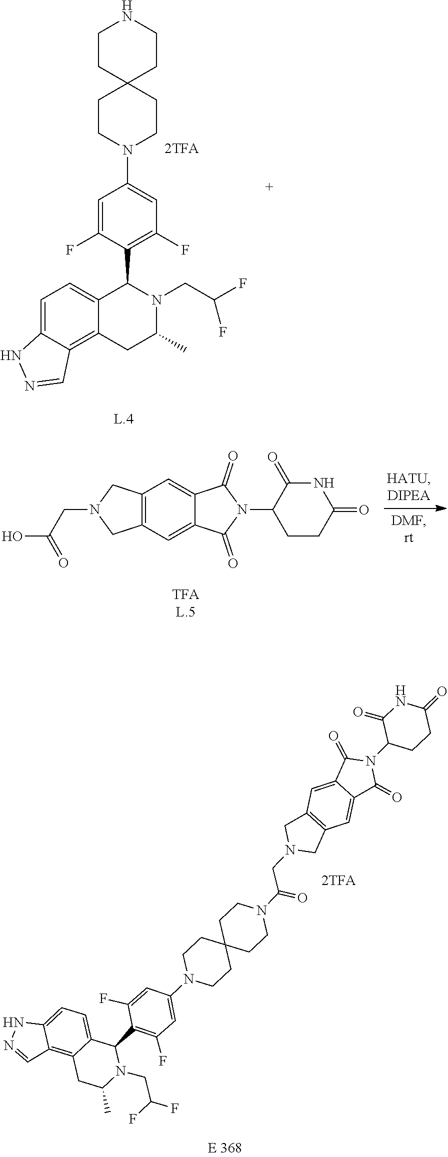 [SMILES: C[C@@H]1Cc2c(ccc3[nH]ncc23)[C@@H](c2c(F)cc(N3CCC4(CCNCC4)CC3)cc2F)N1CC(F)F ; O=C(O)CN1Cc2cc3c(cc2C1)C(=O)N(C1CCC(=O)NC1=O)C3=O ; C[C@@H]1Cc2c(ccc3[nH]ncc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)F]

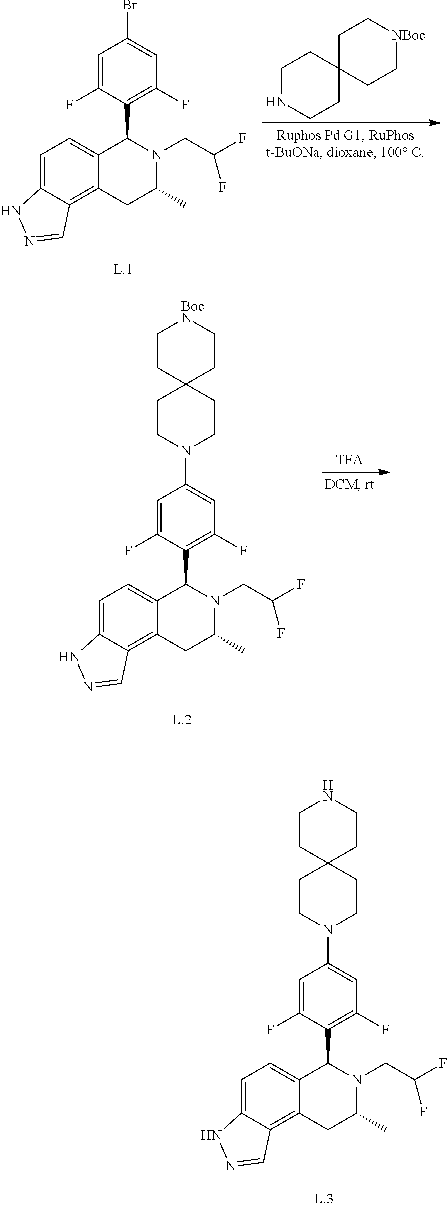 [SMILES: C[C@@H]1Cc2c(ccc3[nH]ncc23)[C@@H](c2c(F)cc(Br)cc2F)N1CC(F)F ; CC(C)(C)OC(=O)N1CCC2(CCNCC2)CC1 ; C[C@@H]1Cc2c(ccc3[nH]ncc23)[C@@H](c2c(F)cc(N3CCC4(CCN(C(=O)OC(C)(C)C)CC4)CC3)cc2F)N1CC(F)F ; C[C@@H]1Cc2c(ccc3[nH]ncc23)[C@@H](c2c(F)cc(N3CCC4(CCNCC4)CC3)cc2F)N1CC(F)F]

To a mixture of intermediate L.1 (trans/cis mixture, 300 mg, 1.0 eq), tert-butyl 3,9-diazaspiro[5.5]undecane-3-carboxylate (345 mg, 2.0 eq), RuPhos Pd G1 (83 mg, 0.15 eq), RuPhos (47.5 mg, 0.15 eq) and t-BuONa (260 mg, 4.0 eq) in dioxane (12 mL) was degassed and purged with N₂ 3 times, and then the mixture was stirred at 100° C. for 6 h. LC-MS showed the starting material L.1 was consumed completely, and a main peak with desired MS was formed. The mixture was cooled, diluted with DCM/MeOH, filtered through Celite, the filter cake was washed with DCM, and the filtrated was concentrated under reduced pressure. The residue was purified by silica gel flash chromatography (PE:EA=100:0 to 60:40), and the crude product (trans/cis mixture) was obtained as a yellow oil. The trans isomer L.2 was separated from the trans/cis mixture by pre-HPLC (ACN/H₂O=55% to 100% in 45 min, 60 mL/min, 61% ACN/H₂O trans isomer come out).

To a solution of L.2 in DCM (5 mL) was added TFA (2 mL). The reaction mixture was stirred for 2 h, then concentrated under reduced pressure. The residue was lyophilized, and compound L.3 (126 mg) was obtained as a white solid.

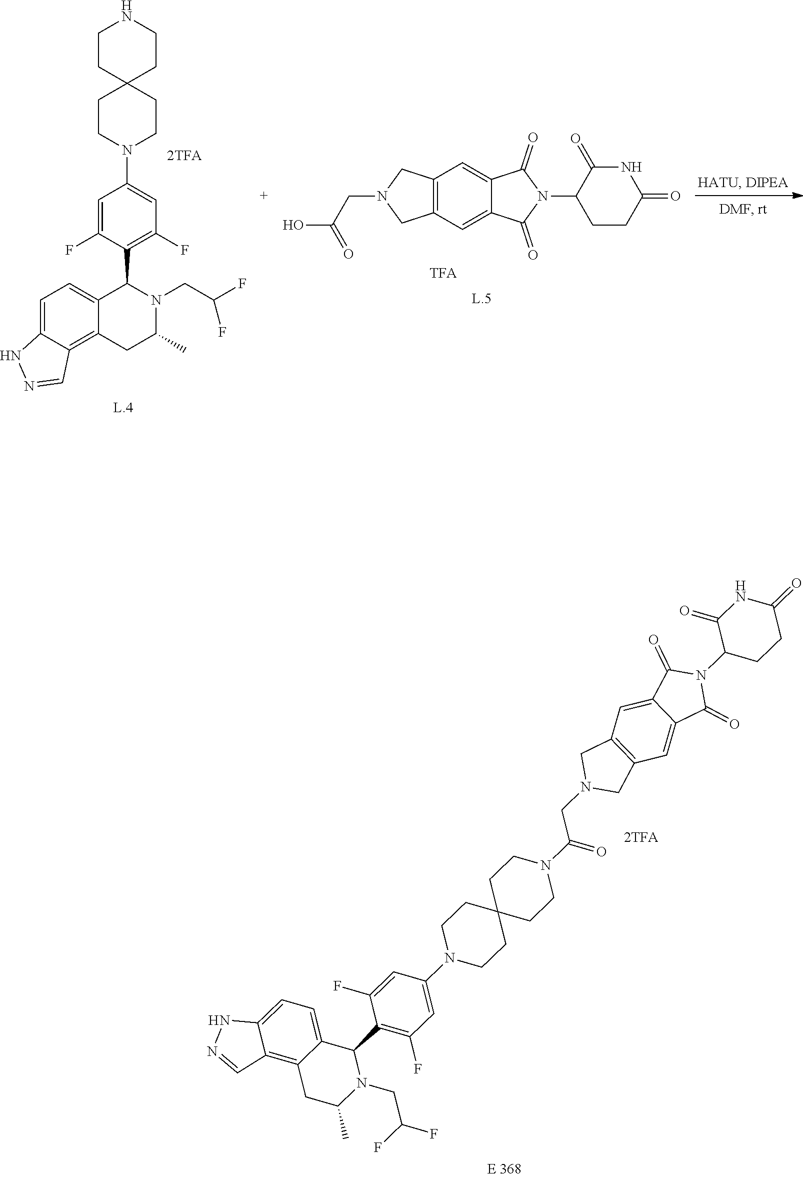 [SMILES: C[C@@H]1Cc2c(ccc3[nH]ncc23)[C@@H](c2c(F)cc(N3CCC4(CCNCC4)CC3)cc2F)N1CC(F)F ; O=C(O)CN1Cc2cc3c(cc2C1)C(=O)N(C1CCC(=O)NC1=O)C3=O ; C[C@@H]1Cc2c(ccc3[nH]ncc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)F]

To a solution of L.4 (49 mg, 1.2 eq) and HATU (33 mg, 1.0 eq) in DMF (2.5 mL) was added DIPEA (91 μL, 6.0 eq). 5 min later, L.5 (65 mg, 1.0 eq) was added, and the reaction mixture was stirred for 10 minutes. Then quenched with water 1 mL, and immediately purified by pre-HPLC (ACN/H₂O (25% to 100%) in 75 min, 60 mL/min), the desired product came out when ACN/H₂O=33.3%. The title compound E 368 (66 mg, 70% yield) was obtained as a white solid after concentrated and lyophilized. LCMS (ESI) m/z: 855.14 [M+1]. ¹H NMR (400 MHz, Methanol-d₄) δ 8.16 (s, 1H), 7.93 (s, 2H), 7.41 (d, J=8.7 Hz, 1H), 6.97 (d, J=8.8 Hz, 1H), 6.56 (d, J=13.7 Hz, 2H), 6.25 (t, J=54.2 Hz, 1H), 5.97 (s, 1H), 5.17 (dd, J=12.6, 5.4 Hz, 1H), 4.96 (s, 4H), 4.61 (s, 2H), 4.14-4.01 (m, 1H), 3.80-3.62 (m, 3H), 3.62-3.53 (m, 1H), 3.46-3.32 (m, 6H), 3.27-3.19 (m, 1H), 2.93-2.68 (m, 3H), 2.21-2.11 (m, 1H), 1.75-1.53 (m, 9H), 1.49 (d, J=6.5 Hz, 3H). ¹³C NMR (101 MHz, MeOD) δ 174.47, 171.31, 167.88, 165.51, 165.41, 163.98, 163.07, 162.97, 155.03, 142.13, 140.89, 134.18, 133.10, 127.43, 126.33, 123.17, 119.53, 110.57, 98.64, 98.37, 60.60, 59.26, 57.56, 53.53, 50.82, 44.23, 41.82, 39.06, 36.23, 35.51, 32.12, 31.04, 30.38, 23.56, 15.19.

### Example 16. Biological Activity and Characterization of Representative Compounds of the Disclosure

ER degradation activity was evaluated in the human breast cancer cell line T47D and MCF7 purchased from the American Type Culture Collection (ATCC), Manassas, VA, and maintained and cultured in Dulbecco's Modified Eagle's medium (DMEM) containing 10% fetal bovine serum, 1 unit/ml of penicillin and 1 μg/ml of streptomycin. Cells with 3-8 passages after purchase were used in experiments.

The protocol for the in-cell western blot analysis: a. seed cells in black-sided/clear bottom 96- or 384-well plates at 40,000 or 10,000 cells/well, overnight; b. add diluted compounds (final 0.5% DMSO), 16 hours. 16 h later, remove medium, add 100 μL or 25 μL of 3.7-4.0% formaldehyde (PBS:FA=9:1), RT 20 min, no shaking; c. wash with PBS, and permeabilized with 100 μL or 25 μL/well of 1×PBS+0.1% Triton X-100 10 minutes; d. block with 100 μL or 25 μL Licor blocking buffer (Li-Cor), RT 1 h, moderate shaking; d. Add 100 μL or 25 μL of anti-ER (cs-8644, 1:500-1,000)+GAPDH(Millipore MAB374, 1:1000) in Block+0.05% Tween 20. RT 2 h, gentle shaking. Negative control: cells plus secondary antibodies (no primary antibodies); e. wash ×4 with PBS+0.05-0.1% Tween 20, gentel shaking; f. anti-rabbit-680 and anti-mouse-800 (both 1:1000 in LiCor block+0.05% Tween20, RT 1 h, gentle shaking, no light. LI-COR: 0.2% to reduce background; g. wash×4 with PBS+0.05% Tween 20, gental shaking; h. add 100 μL or 25 μL of PBS to each well and read on CLX plate reader. The relative ER percentage in treated cells were obtained by comparing the values of treated wells to those in untreated and DMSO-treated wells as 100%.

Table A provides the ER degradation activity, LCMS analytical characterization data, and general synthetic scheme used for the synthesis of representative Compounds of the Disclosure.

TABLE A

| | Potency | LC-MS | General Synthetic |
|---|---|---|---|
| Cpd. No. | (DC₅₀) | ([M + H]+) | Scheme # |
| E 1 | C | 681.34 | 1 |
| E 2 | B | 695.32 | 1 |
| E 3 | A | 764.41 | 2 |
| E 4 | B | 764.37 | 3 |
| E 5 | A | 750.36 | 3 |
| E 6 | A | 736.34 | 3 |
| E 7 | B | 778.35 | 3 |
| E 8 | B | 736.23 | 3 |
| E 9 | A | 764.34 | 3 |
| E 10 | B | 764.34 | 3 |
| E 11 | B | 816.29 | 3 |
| E 12 | A | 810.36 | 5 |
| E 13 | A | 792.37 | 6 |
| E 14 | B | 806.32 | 6 |
| E 15 | B | 764.34 | 6 |
| E 16 | B | 778.35 | 6 |
| E 17 | B | 750.32 | 3 |
| E 18 | B | 754.30 | 3 |
| E 19 | A | 781.92 | 3 |
| E 20 | B | 792.43 | 3 |
| E 21 | C | 739.31 | 7 |
| E 22 | C | 779.34 | 7 |
| E 23 | B | 807.37 | 7 |
| E 24 | B | 843.33 | 7 |
| E 25 | C | 817.85 | 13 |
| E 26 | C | 803.87 | 13 |
| E 27 | C | 803.87 | 12 |
| E 28 | C | 803.87 | 12 |
| E 29 | C | 845.91 | 14 |
| E 30 | C | 831.93 | 14 |
| E 31 | C | 817.85 | 13 |
| E 32 | C | 803.87 | 13 |
| E 33 | C | 831.88 | 13 |
| E 34 | B | 817.90 | 13 |
| E 35 | A | 789.84 | 12 |
| E 36 | A | 775.82 | 12 |
| E 37 | A | 775.82 | 12 |
| E 38 | B | 775.82 | 13 |
| E 39 | A | 789.84 | 13 |
| E 40 | A | 789.84 | 13 |
| E 41 | A | 803.31 | 12 |
| E 42 | A | 775.28 | 12 |
| E 43 | A | 789.29 | 12 |
| E 44 | B | 789.29 | 12 |
| E 45 | A | 803.31 | 12 |
| E 46 | C | 789.33 | 15 |
| E 47 | C | 789.33 | 15 |
| E 48 | B | 752.32 | 9 |
| E 49 | B | 766.30 | 9 |
| E 50 | C | 812.88 | 19 |
| E 51 | B | 840.94 | 18 |
| E 52 | A | 798.37 | 19 |
| E 53 | B | 826.95 | 18 |
| E 54 | B | 758.83 | 19 |
| E 55 | C | 840.89 | 20 |
| E 56 | B | 826.91 | 20 |
| E 57 | A | 854.92 | 21 |
| E 58 | B | 868.95 | 21 |
| E 59 | B | 840.94 | 21 |
| E 60 | C | 798.29 | 17 |
| E 61 | C | 784.32 | 17 |
| E 62 | A | 840.34 | 17 |
| E 63 | A | 826.36 | 17 |
| E 64 | A | 826.36 | 16 |
| E 65 | A | 783.98 | 17 |
| E 66 | A | 798.33 | 16 |
| E 67 | B | 825.36 | 16 |
| E 68 | B | 812.22 | 16 |
| E 69 | C | 812.42 | 16 |
| E 70 | B | 812.54 | 16 |
| E 71 | A | 784.28 | 16 |
| E 72 | A | 798.02 | 16 |
| E 73 | A | 798.51 | 17 |
| E 74 | B | 812.38 | 17 |
| E 75 | B | 811.25 | 17 |
| E 76 | C | 826.33 | 17 |
| E 77 | A | 812.31 | 16 |
| E 78 | C | 840.48 | 16 |
| E 79 | B | 826.28 | 16 |
| E 80 | B | 826.33 | 16 |
| E 81 | B | 826.33 | 16 |
| E 82 | A | 798.29 | 16 |
| E 83 | A | 812.07 | 16 |
| E 84 | A | 826.27 | 16 |
| E 85 | A | 812.46 | 16 |
| E 86 | B | 826.40 | 22 |
| E 87 | C | 826.40 | 22 |
| E 88 | C | 743.29 | 22 |
| E 89 | C | 757.30 | 22 |
| E 90 | A | 840.38 | 21 |
| E 91 | A | 854.36 | 21 |
| E 92 | A | 854.39 | 21 |
| E 93 | A | 868.38 | 23 |
| E 94 | A | 854.39 | 23 |
| E 95 | A | 869.37 | 23 |
| E 96 | A | 855.39 | 23 |
| E 97 | B | 826.33 | 17 |
| E 98 | A | 812.35 | 17 |
| E 99 | B | 908.40 | 23 |
| E 100 | A | 894.39 | 23 |
| E 101 | A | 880.41 | 23 |
| E 102 | C | 743.29 | 23 |
| E 103 | B | 798.33 | 23 |
| E 104 | C | 771.32 | 23 |
| E 105 | C | 826.33 | 17 |
| E 106 | B | 812.35 | 17 |
| E 107 | A | 840.34 | 17 |
| E 108 | A | 826.36 | 17 |
| E 109 | A | 869.58 | 24 |
| E 110 | C | 840.40 | 20 |
| E 111 | A | 826.42 | 16 |
| E 112 | A | 854.36 | 16 |
| E 113 | B | 868.37 | 23 |
| E 114 | A | 854.36 | 23 |
| E 115 | A | 880.37 | 23 |
| E 116 | A | 866.36 | 23 |
| E 117 | B | 812.35 | 22 |
| E 118 | C | 840.38 | 22 |
| E 119 | C | 826.33 | 21 |
| E 120 | B | 812.35 | 21 |
| E 121 | A | 840.34 | 23 |
| E 122 | A | 826.33 | 23 |
| E 123 | B | 812.35 | 18 |
| E 124 | C | 826.36 | 22 |
| E 125 | B | 764.04 | 4 |
| E 126 | B | 793.49 | 4 |
| E 127 | B | 782.51 | 4 |
| E 128 | A | 854.39 | 18 |
| E 129 | A | 826.36 | 18 |
| E 130 | A | 854.39 | 18 |
| E 131 | A | 852.38 | 18 |
| E 132 | B | 880.41 | 18 |
| E 133 | B | 894.43 | 18 |
| E 134 | B | 798.33 | 23 |
| E 135 | A | 840.9 | 16 |
| E 136 | B | 841.37 | 19 |
| E 137 | B | 827.39 | 19 |
| E 138 | B | 817.36 | 12 |
| E 139 | B | 778.35 | 3 |
| E 140 | C | 778.35 | 4 |
| E 141 | C | 778.35 | 4 |
| E 142 | B | 792.37 | 3 |
| E 143 | B | 792.37 | 3 |
| E 144 | C | 792.37 | 4 |
| E 145 | B | 810.36 | 3 |
| E 146 | C | 810.36 | 4 |
| E 147 | C | 743.33 | 18 |
| E 148 | A | 764.12 | 3 |
| E 149 | A | 792.22 | 3 |
| E 150 | C | 764.34 | 4 |
| E 151 | C | 792.37 | 4 |
| E 152 | C | 796.34 | 4 |
| E 153 | C | 796.34 | 4 |
| E 154 | C | 796.34 | 4 |
| E 155 | C | 814.34 | 4 |
| E 156 | B | 778.35 | 6 |
| E 157 | C | 757.30 | 7 |
| E 158 | C | 750.32 | 6 |
| E 159 | C | 757.30 | 7 |
| E 160 | C | 749.36 | 1 |
| E 161 | C | 806.38 | 4 |
| E 162 | B | 806.38 | 3 |
| E 163 | B | 778.35 | 3 |
| E 164 | B | 796.34 | 3 |
| E 165 | B | 824.37 | 3 |
| E 166 | C | 820.40 | 3 |
| E 167 | C | 820.40 | 4 |
| E 168 | C | 792.37 | 4 |
| E 169 | C | 792.37 | 3 |
| E 170 | C | 792.37 | 4 |
| E 171 | C | 792.37 | 3 |
| E 172 | B | 808.40 | 3 |
| E 173 | C | 782.33 | 3 |
| E 174 | B | 782.33 | 3 |
| E 175 | C | 825.38 | 3 |
| E 176 | C | 828.35 | 3 |
| E 177 | B | 798.42 | 3 |
| E 178 | B | 834.40 | 3 |
| E 179 | B | 770.38 | 3 |
| E 180 | B | 820.23 | 26 |
| E 181 | C | 820.05 | 26 |
| E 182 | A | 826.07 | 27 |
| E 183 | C | 837.39 | 3 |
| E 184 | C | 810.36 | 3 |
| E 185 | C | 811.35 | 3 |
| E 186 | B | 796.34 | 3 |
| E 187 | B | 798.03 | 27 |
| E 188 | B | 792.15 | 27 |
| E 189 | B | 778.25 | 27 |
| E 190 | B | 812.15 | 27 |
| E 191 | B | 812.18 | 27 |
| E 192 | B | 778.11 | 27 |
| E 193 | B | 858.09 | 27 |
| E 194 | B | 778.07 | 27 |
| E 195 | B | 826.09 | 27 |
| E 196 | B | 844.20 | 27 |
| E 197 | B | 854.11 | 28 |
| E 198 | B | 854.12 | 28 |
| E 199 | C | 796.34 | 6 |
| E 200 | C | 750.32 | 6 |
| E 201 | C | 778.35 | 6 |
| E 202 | C | 814.33 | 6 |
| E 203 | C | 796.34 | 6 |
| E 204 | B | 806.38 | 6 |
| E 205 | C | 806.38 | 21 |
| E 206 | C | 824.37 | 6 |
| E 207 | C | 796.34 | 6 |
| E 208 | C | 824.37 | 6 |
| E 209 | B | 858.33 | 6 |
| E 210 | C | 876.32 | 6 |
| E 211 | C | 810.40 | 2 |
| E 212 | C | 782.36 | 2 |
| E 213 | B | 810.36 | 3 |
| E 214 | C | 789.33 | 3 |
| E 215 | C | 761.31 | 3 |
| E 216 | A | 840.34 | 23 |
| E 217 | C | 792.40 | 2 |
| E 218 | C | 764.37 | 2 |
| E 219 | C | 739.31 | 10 |
| E 220 | C | 779.34 | 10 |
| E 221 | C | 751.31 | 11 |
| E 222 | C | 779.34 | 11 |
| E 223 | C | 779.34 | 10 |
| E 224 | C | 807.37 | 10 |
| E 225 | C | 849.33 | 12 |
| E 226 | B | 872.35 | 16 |
| E 227 | B | 844.32 | 16 |
| E 228 | B | 872.35 | 23 |
| E 229 | B | 798.28 | 23 |
| E 230 | B | 812.31 | 23 |
| E 231 | C | 843.36 | 25 |
| E 232 | C | 797.34 | 25 |
| E 233 | C | 868.37 | 23 |
| E 234 | B | 840.34 | 23 |
| E 235 | C | 826.33 | 23 |
| E 236 | C | 826.33 | 23 |
| E 237 | C | 840.34 | 23 |
| E 238 | C | 868.38 | 21 |
| E 239 | C | 840.34 | 21 |
| E 240 | C | 792.37 | 6 |
| E 241 | C | 810.36 | 6 |
| E 242 | B | 764.31 | 27 |
| E243 | A | 750.28 | 29 |
| E244 | B | 764.16 | 29 |
| E245 | C | 782.13 | 29 |
| E246 | A | 852.98 | 30 |
| E247 | B | 885.14 | 30 |
| E248 | C | 867.11 | 30 |
| E249 | C | 825.18 | 30 |
| E250 | A | 839.02 | 30 |
| E 251 | A | 840.42 | 18 |
| E 252 | A | 868.37 | 23 |
| E 253 | A | 868.37 | 16 |
| E 254 | A | 838.37 | 16 |
| E 255 | A | 810.33 | 16 |
| E 256 | B | 824.38 | 18 |
| E 257 | A | 838.36 | 23 |
| E 258 | A | 820.37 | 16 |
| E 259 | A | 792.34 | 16 |
| E 260 | A | 820.37 | 16 |
| E 261 | B | 806.39 | 18 |
| E 262 | B | 790.35 | 16 |
| E 263 | B | 818.35 | 16 |
| E 264 | B | 804.40 | 18 |
| E 265 | B | 818.38 | 16 |
| E 266 | B | 750.36 | 31 |
| E 267 | B | 750.36 | 31 |
| E 268 | B | 778.39 | 31 |
| E 269 | C | 831.34 | 31 |
| E 270 | C | 817.36 | 15 |
| E 271 | A | 778.28 | 29 |
| E 272 | C | 750.30 | 29 |
| E 273 | B | 796.17 | 29 |
| E 274 | C | 782.21 | 29 |
| E 275 | A | 782.22 | 29 |
| E 276 | C | 768.19 | 29 |
| E 277 | C | 782.16 | 29 |
| E 278 | C | 754.09 | 29 |
| E 279 | B | 754.32 | 29 |
| E 280 | C | 819.37 | 16 |
| E 281 | B | 819.37 | 16 |
| E 282 | C | 839.48 | 30 |
| E 283 | C | 825.12 | 30 |
| E 284 | C | 792.40 | 2 |
| E 285 | C | 768.18 | 29 |
| E 286 | C | 782.20 | 29 |
| E 287 | B | 820.40 | 33 |
| E 288 | B | 806.42 | 33 |
| E 289 | C | 806.20 | 34 |
| E 290 | B | 778.16 | 34 |
| E 291 | C | 792.14 | 34 |
| E 292 | C | 819.37 | 32 |
| E 293 | B | 836.37 | 16 |
| E 294 | C | 737.00 | 29 |
| E 295 | C | 778.20 | 29 |
| E 296 | C | 764.26 | 29 |
| E 297 | C | 793.14 | 40 |
| E 298 | C | 794.30 | 40 |
| E 299 | B | 778.20 | 34 |
| E 300 | C | 792.44 | 33 |
| E 301 | B | 795.36 | 12 |
| E 302 | A | 836.37 | 30 |
| E 303 | C | 778.12 | 34 |
| E 304 | C | 794.24 | 40 |
| E 305 | C | 793.04 | 40 |
| E 306 | C | 794.08 | 40 |
| E 307 | A | 839.01 | 30 |
| E 308 | B | 825.00 | 30 |
| E 309 | B | 849.11 | 30 |
| E 310 | C | 793.35 | 33 |
| E 311 | C | 773.34 | 41 |
| E 312 | B | 835.37 | 30 |
| E 313 | B | 818.38 | 30 |
| E 314 | C | 816.36 | 30 |
| E 315 | C | 817.37 | 30 |
| E 316 | C | 815.37 | 30 |
| E 317 | B | 812.36 | 30 |
| E 318 | A | 795.34 | 12 |
| E 319 | C | 818.38 | 30 |
| E 320 | A | 831.40 | 12 |
| E 321 | A | 795.34 | 31 |
| E 322 | C | 796.04 | 12 |
| E 323 | A | 812.44 | 12 |
| E 324 | C | 792.40 | 34 |
| E 325 | B | 847.96 | 30 |
| E326 | C | 834.13 | 30 |
| E327 | C | 761.15 | 29 |
| E328 | C | 760.15 | 29 |
| E329 | B | 760.15 | 41 |
| E330 | C | 761.04 | 41 |
| E331 | C | 738.03 | 42 |
| E332 | C | 752.20 | 42 |
| E333 | C | 778.28 | 34 |
| E334 | C | 778.25 | 34 |
| E 335 | C | 778.25 | 34 |
| E 336 | C | 778.07 | 42 |
| E 337 | C | 764.03 | 34 |
| E 338 | C | 764.53 | 34 |
| E 339 | C | 764.25 | 34 |
| E 340 | C | 750.24 | 34 |
| E 341 | C | 750.23 | 34 |
| E 342 | C | 760.91 | 41 |
| E 343 | A | 847.92 | 30 |
| E 344 | C | 849.05 | 30 |
| E 345 | B | 849.07 | 30 |
| E 346 | A | 849.13 | 30 |
| E 347 | A | 846.97 | 30 |
| E 348 | B | 853.06 | 30 |
| E 349 | C | 799.31 | 32 |
| E 350 | C | 781.05 | 32 |
| E 351 | C | 792.18 | 32 |
| E 352 | C | 831.04 | 12 |
| E 353 | C | 831.08 | 12 |
| E 354 | A | 778.24 | 4 |
| E 355 | C | 778.30 | 33 |
| E 356 | C | 778.22 | 33 |
| E 357 | A | 765.12 | 33 |
| E 358 | C | 765.20 | 33 |
| E 359 | B | 764.22 | 33 |
| E 360 | A | 820.24 | 33 |
| E 361 | C | 820.27 | 33 |
| E 362 | C | 792.19 | 33 |
| E 363 | A | 792.17 | 33 |
| E 364 | B | 791.99 | 33 |
| E 365 | C | 806.18 | 33 |
| E 366 | C | 839.26 | 30 |
| E 367 | C | 839.14 | 30 |
| E 368 | A | 855.11 | 44 |
| E 369 | C | 825.05 | 43 |
| E 370 | C | 811.10 | 43 |
| E 371 | A | 854.06 | 45 |
| E 372 | A | 854.10 | 45 |
| E 373 | C | 759.13 | 41 |
| E 374 | C | 764.23 | 34 |
| E 375 | C | 764.20 | 34 |
| E 376 | C | 822.11 | 41 |
| E 377 | B | 794.15 | 34 |
| E 378 | C | 852.97 | 43 |
| E 379 | C | 852.98 | 43 |
| E 380 | C | 839.08 | 43 |
| E 381 | C | 839.19 | 43 |
| E 382 | C | 853.12 | 43 |
| E 383 | C | 853.08 | 43 |
| E 384 | B | 779.10 | 41 |
| E 385 | A | 779.10 | 41 |
| E 386 | C | 778.14 | 41 |
| E 387 | A | 778.12 | 41 |
| E 388 | B | 779.11 | 33 |
| E 389 | C | 779.13 | 33 |
| E 390 | B | 778.19 | 33 |
| E 391 | C | 778.19 | 33 |
| E 392 | B | 853.08 | 30 |
| E 393 | A | 852.97 | 30 |
| E 394 | C | 792.23 | 33 |
| E 395 | C | 792.23 | 33 |
| E 396 | C | 848.10 | 30 |
| E 397 | C | 848.13 | 30 |
| E 398 | C | 806.21 | 33 |
| E 399 | C | 834.22 | 30 |
| E 400 | C | 883.13 | 30 |
| E 402 | C | 848.13 | 30 |
| E 403 | B | 847.20 | 30 |
| E 404 | C | 847.24 | 30 |
| E 405 | C | 848.30 | 30 |
| E 406 | C | 835.99 | 30 |
| E 407 | C | 840.11 | 45 |
| E 408 | A | 827.23 | 44 |
| E 409 | B | 855.12 | 44 |
| E 410 | A | 855.12 | 45 |
| E 411 | C | 854.25 | 46 |
| E 412 | C | 854.08 | 46 |
| E 413 | A | 838.35 | 44 |
| E 414 | B | 837.35 | 45 |
| E 415 | A | 835.31 | 45 |
| E 416 | B | 841.10 | 44 |
| E 417 | A | 827.32 | 44 |
| E 418 | C | 798.32 | 44 |
| E 419 | A | 885.03 | 47 |
| E 420 | B | 822.14 | 3 |
| E 421 | A | 841.01 | 47 |
| E 422 | C | 816.40 | 48 |
| E 423 | B | 816.37 | 48 |
| E 424 | B | 841.39 | 44 |
| E 425 | A | 841.36 | 44 |
| E 426 | A | 750.33 | 3 |
| E 427 | A | 813.32 | 44 |
| E 428 | B | 813.32 | 44 |
| E 429 | A | 778.28 | 3 |
| E 430 | A | 848.32 | 16 |
| E 431 | A | 849.41 | 44 |
| E 432 | A | 835.40 | 44 |
| E 433 | C | 838.33 | 44 |
| E 434 | B | 820.32 | 44 |
| E 435 | C | 820.19 | 44 |
| E 436 | B | 849.33 | 44 |
| E 437 | C | 792.37 | 3 |
| E 438 | C | 764.34 | 3 |
| E 439 | A | 808.37 | 31 |
| E 440 | A | 871.48 | 44 |
| E 441 | A | 843.18 | 44 |
| E 442 | | 854.4 | 44 |
| E 443 | | 824.34 | 44 |
| E 444 | A | 763.45 | 16 |
| E 445 | | 836.35 | 16 |
| E 446 | C | 828.00 | 12 |
| E 447 | C | 830.14 | 12 |
| E 448 | C | 792.06 | 12 |
| E 449 | C | 791.97 | 12 |
| E 450 | B | 794.06 | 12 |
| E 451 | B | 817.36 | 12 |
| E 452 | B | 831.06 | 12 |
| E 453 | A | 832.04 | 11 |
| E 454 | C | 794.15 | 35 |
| E 455 | B | 818.06 | 11 |
| E 456 | C | 818.04 | 11 |
| E 457 | B | 831.05 | 35 |
| E 458 | C | 831.06 | 35 |
| E 459 | A | 795.02 | 35 |
| E 460 | B | 795.04 | 35 |
| E 461 | A | 847.40 | 12 |
| E 462 | B | 825.33 | 36 |
| E 463 | A | 831.13 | 14 |
| E 464 | C | 802.09 | 35 |
| E 465 | C | 802.01 | 35 |
| E 466 | C | 817.37 | 14 |
| E 467 | C | 794.36 | 38 |
| E 468 | B | 852.30 | 30 |
| E 469 | B | 881.36 | 23 |
| E 470 | B | 840.34 | 44 |
| E 471 | B | 773.40 | 18 |
| E 472 | B | 763.34 | 18 |
| E 473 | B | 773.42 | 18 |
| E 474 | B | 763.36 | 18 |
| E 475 | B | 835.35 | 30 |
| E 476 | B | 851.36 | 30 |
| E 477 | C | 833.34 | 30 |
| E 478 | C | 814.36 | 30 |
| E 479 | C | 835.37 | 30 |
| E 480 | B | 853.38 | 30 |
| E 481 | B | 843.38 | 30 |
| E 482 | C | 847.38 | 30 |
| E 483 | C | 848.94 | 30 |
| E 484 | C | 844.40 | 30 |
| E 485 | C | 859.37 | 30 |
| E 486 | C | 841.38 | 30 |
| E 487 | B | 830.38 | 30 |
| E 488 | C | 806.40 | 6 |
| E 489 | B | 739.32 | 7 |
| E 490 | B | 779.92 | 7 |
| E 491 | B | 779.95 | 29 |
| E 492 | C | 791.38 | 39 |
| E 493 | C | 763.34 | 39 |
| E 494 | C | 763.33 | 39 |
| E 495 | C | 820.38 | 39 |
| E 496 | C | 792.92 | 39 |
| E 497 | C | 804.40 | 29 |
| E 498 | B | 793.39 | 7 |
| E 499 | B | 779.38 | 7 |
| E 500 | C | 735.36 | 37B |
| E 501 | C | 749.36 | 37B |
| E 502 | C | 751.32 | 37B |
| E 503 | C | 751.30 | 37B |
| E 504 | C | 764.38 | 37A |
| E 505 | C | 750.38 | 37A |
| E 506 | C | 778.90 | 37A |
| E 507 | C | 777.45 | 37A |
| E 508 | C | 764.38 | 37A |
| E 509 | C | 856.42 | 4 |
| E 510 | C | 842.40 | 4 |
| E 511 | B | 794.36 | 4 |
| E 512 | C | 793.36 | 18 |
| E 513 | B | 793.32 | 18 |
| E 514 | B | 858.43 | 16 |
| E 515 | B | 751.31 | 1 |
| E 516 | — | 737.36 | 1 |
| E 517 | B | 767.32 | 1 |
| E 518 | — | 753.37 | 1 |
| E 519 | C | 707.39 | 1 |

DC₅₀: “A”: <10 nM;
“B”: 10-100 nM;
“C”: >100 nM

### REFERENCES

(1) Tong, C. W. S.; Wu, M.; Cho, W. C. S.; To, K. K. W. Recent advances in the treatment of breast cancer. Front. Oncol. 2018, 8.
(2) Anderson, W. F.; Katki, H. A.; Rosenberg, P. S. Incidence of breast cancer in the United States: current and future trends. J. Natl. Cancer Inst. 2011, 103, 1397-1402.
(3) Nilsson, S.; Koehler, K. F.; Gustafsson, J. A. Development of subtype-selective oestrogen receptor-based therapeutics. Nat. Rev. Drug Discovery 2011, 10, 778-792.
(4) Jordan, V. C. Tamoxifen: a most unlikely pioneering medicine. Nat. Rev. Drug Discovery 2003, 2, 205-213.
(5) Das, S.; Crockett, J. C. Osteoporosis—a current view of pharmacological prevention and treatment. Drug Des. Devel. Ther. 2013, 7, 435-448.
(6) De Marchi, T.; Foekens, J. A.; Umar, A.; Martens, J. W. Endocrine therapy resistance in estrogen receptor (ER)-positive breast cancer. Drug Discovery Today 2016, 21, 1181-1188.
(7) AlFakeeh, A.; Brezden-Masley, C. Overcoming endocrine resistance in hormone receptor-positive breast cancer. Curr. Oncol. 2018, 25, S18-S27.
(8) Martin, L. A.; Ribas, R.; Simigdala, N.; Schuster, E.; Pancholi, S.; Tenev, T.; Gellert, P.; Buluwela, L.; Harrod, A.; Thornhill, A.; Nikitorowicz-Buniak, J.; Bhamra, A.; Turgeon, M. O.; Poulogiannis, G.; Gao, Q.; Martins, V.; Hills, M.; Garcia-Murillas, I.; Fribbens, C.; Patani, N.; Li, Z.; Sikora, M. J.; Turner, N.; Zwart, W.; Oesterreich, S.; Carroll, J.; Ali, S.; Dowsett, M. Discovery of naturally occurring ESR1 mutations in breast cancer cell lines modelling endocrine resistance. Nat. Commun. 2017, 8, 1865.
(9) Nardone, A.; De Angelis, C.; Trivedi, M. V.; Osborne, C. K.; Schiff, R. The changing role of ER in endocrine resistance. Breast 2015, 24, S60-S66.
(10) Robertson, J. F.; Harrison, M. Fulvestrant: pharmacokinetics and pharmacology. Br. J. Cancer 2004, 90, S7-S10.
(11) Osborne, C. K.; Wakeling, A.; Nicholson, R. I. Fulvestrant: an oestrogen receptor antagonist with a novel mechanism of action. Br. J. Cancer 2004, 90, S2-S6.
(12) Howell, A.; Sapunar, F. Fulvestrant revisited: efficacy and safety of the 500-mg dose. Clin. Breast Cancer 2011, 11, 204-210.
(13) Robertson, J. F.; Lindemann, J.; Garnett, S.; Anderson, E.; Nicholson, R. I.; Kuter, I.; Gee, J. M. A good drug made better: the fulvestrant dose-response story. Clin. Breast Cancer 2014, 14, 381-389.
(14) McDonnell, D. P.; Wardell, S. E.; Norris, J. D. Oral selective estrogen receptor downregulators (SERDs), a breakthrough endocrine therapy for breast cancer. J. Med. Chem. 2015, 58, 4883-4887.
(15) Abdel-Magid, A. F. Selective estrogen receptor degraders (SERDs): a promising treatment to overcome resistance to endocrine therapy in ERα-positive breast cancer. ACS Med. Chem. Lett. 2017, 8, 1129-1131.
(16) Weir, H. M.; Bradbury, R. H.; Lawson, M.; Rabow, A. A.; Buttar, D.; Callis, R. J.; Curwen, J. O.; de Almeida, C.; Ballard, P.; Hulse, M.; Donald, C. S.; Feron, L. J.; Karoutchi, G.; MacFaul, P.; Moss, T.; Norman, R. A.; Pearson, S. E.; Tonge, M.; Davies, G.; Walker, G. E.; Wilson, Z.; Rowlinson, R.; Powell, S.; Sadler, C.; Richmond, G.; Ladd, B.; Pazolli, E.; Mazzola, A. M.; D'Cruz, C.; De Savi, C. AZD9496: an oral estrogen receptor inhibitor that blocks the growth of ER-positive and ESR1-mutant breast tumors in preclinical models. Cancer Res. 2016, 76, 3307-3318.
(17) Joseph, J. D.; Darimont, B.; Zhou, W.; Arrazate, A.; Young, A.; Ingalla, E.; Walter, K.; Blake, R. A.; Nonomiya, J.; Guan, Z.; Kategaya, L.; Govek, S. P.; Lai, A. G.; Kahraman, M.; Brigham, D.; Sensintaffar, J.; Lu, N.; Shao, G.; Qian, J.; Grillot, K.; Moon, M.; Prudente, R.; Bischoff, E.; Lee, K. J.; Bonnefous, C.; Douglas, K. L.; Julien, J. D.; Nagasawa, J. Y.; Aparicio, A.; Kaufman, J.; Haley, B.; Giltnane, J. M.; Wertz, I. E.; Lackner, M. R.; Nannini, M. A.; Sampath, D.; Schwarz, L.; Manning, H. C.; Tantawy, M. N.; Arteaga, C. L.; Heyman, R. A.; Rix, P. J.; Friedman, L.; Smith, N. D.; Metcalfe, C.; Hager, J. H. The selective estrogen receptor downregulator GDC-0810 is efficacious in diverse models of ER+ breast cancer. eLife 2016, 5, e15828.
(18) Bihani, T.; Patel, H. K.; Arlt, H.; Tao, N.; Jiang, H.; Brown, J. L.; Purandare, D. M.; Hattersley, G.; Garner, F. Elacestrant (RAD1901), a selective estrogen receptor degrader (SERD), has antitumor activity in multiple ER+ breast cancer patient-derived xenograft models. Clin. Cancer Res. 2017, 23, 4793-4804.
(19) Tria, G. S.; Abrams, T.; Baird, J.; Burks, H. E.; Firestone, B.; Gaither, L. A.; Hamann, L. G.; He, G.; Kirby, C. A.; Kim, S.; Lombardo, F.; Macchi, K. J.; McDonnell, D. P.; Mishina, Y.; Norris, J. D.; Nunez, J.; Springer, C.; Sun, Y.; Thomsen, N. M.; Wang, C.; Wang, J.; Yu, B.; Tiong-Yip, C. L.; Peukert, S. Discovery of LSZ102, a potent, orally bioavailable selective estrogen receptor degrader (SERD) for the treatment of estrogen receptor positive breast cancer. J. Med. Chem. 2018, 61, 2837-2864.
(20) Carlson, R. W. The history and mechanism of action of fulvestrant. Clin. Breast Cancer 2005, 6, S5-S8.
(21) Marsaud, V.; Gougelet, A.; Maillard, S.; Renoir, J. M. Various phosphorylation pathways, depending on agonist and antagonist binding to endogenous estrogen receptor a (ERα), differentially affect ERα extractability, proteasome-mediated stability, and transcriptional activity in human breast cancer cells. Mol. Endocrinol. 2003, 17, 2013-2027.
(22) Wittmann, B. M.; Sherk, A.; McDonnell, D. P. Definition of functionally important mechanistic differences among selective estrogen receptor down-regulators. Cancer Res. 2007, 67, 9549-9560.
(23) Sakamoto, K. M.; Kim, K. B.; Kumagai, A.; Mercurio, F.; Crews, C. M.; Deshaies, R. J. Protacs: chimeric molecules that target proteins to the Skpl-Cullin-F box complex for ubiquitination and degradation. Proc. Natl. Acad. Sci. U.S.A 2001, 98, 8554-8559.
(24) Toure, M.; Crews, C. M. Small-molecule PROTACS: new approaches to protein degradation. Angew. Chem., Int. Ed. 2016, 55, 1966-1973.
(25) Lai, A. C.; Crews, C. M. Induced protein degradation: an emerging drug discovery paradigm. Nat. Rev. Drug Discovery 2017, 16, 101-114.
(26) Burslem, G. M.; Crews, C. M. Small-molecule modulation of protein homeostasis.

Chem. Rev. 2017, 117, 11269-11301.
(27) Cromm, P. M.; Crews, C. M. Targeted protein degradation: from chemical biology to drug discovery. Cell Chem. Biol. 2017, 24, 1181-1190.
(28) Ottis, P.; Crews, C. M. Proteolysis-targeting chimeras: induced protein degradation as a therapeutic strategy. ACS Chem. Biol. 2017, 12, 892-898.
(29) Rodriguez-Gonzalez, A.; Cyrus, K.; Salcius, M.; Kim, K.; Crews, C. M.; Deshaies, R. J.; Sakamoto, K. M. Targeting steroid hormone receptors for ubiquitination and degradation in breast and prostate cancer. Oncogene 2008, 27, 7201-7211.
(30) Jiang, Y.; Deng, Q.; Zhao, H.; Xie, M.; Chen, L.; Yin, F.; Qin, X.; Zheng, W.; Zhao, Y.; Li, Z. Development of stabilized peptide-based PROTACs against estrogen receptor a. ACS Chem. Biol. 2018, 13, 628-635.
(31) Winter, G. E. B., D. L.; Paulk, J.; Roberts, J. M.; Souza, A.; Dhe-Paganon, S. B., J. E. Phthalimide conjugation as a strategy for in vivo target protein degradation. Science 2015, 348, 1376-1381.
(32) Lu, J.; Qian, Y.; Altieri, M.; Dong, H.; Wang, J.; Raina, K.; Hines, J.; Winkler, J. D.; Crew, A. P.; Coleman, K.; Crews, C. M. Hijacking the E3 ubiquitin ligase cereblon to efficiently target BRD4. Chem. Biol. 2015, 22, 755-763.
(33) Bondeson, D. P.; Mares, A.; Smith, I E.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M. Catalytic in vivo protein knockdown by small-molecule PROTACs. Nat. Chem. Biol. 2015, 11, 611-617.
(34) Lai, A. C.; Toure, M.; Hellerschmied, D.; Salami, J.; Jaime-Figueroa, S.; Ko, E.; Hines, J.; Crews, C. M. Modular PROTAC design for the degradation of oncogenic BCR-ABL. Angew. Chem., Int. Ed. 2016, 55, 807-810.
(35) Robb, C. M.; Contreras, J. I.; Kour, S.; Taylor, M. A.; Abid, M.; Sonawane, Y. A.; Zahid, M.; Murry, D. J.; Natarajan, A.; Rana, S. Chemically induced degradation of CDK9 by a proteolysis targeting chimera (PROTAC). Chem. Commun. 2017, 53, 7577-7580.
(36) Zhang, C.; Han, X. R.; Yang, X.; Jiang, B.; Liu, J.; Xiong, Y.; Jin, J. Proteolysis targeting chimeras (PROTACs) of anaplastic lymphoma kinase (ALK). Eur. J. Med. Chem. 2018, 151, 304-314.
(37) Lu, M.; Liu, T.; Jiao, Q.; Ji, J.; Tao, M.; Liu, Y.; You, Q.; Jiang, Z. Discovery of a Keap1-dependent peptide PROTAC to knockdown Tau by ubiquitination-proteasome degradation pathway. Eur. J. Med. Chem. 2018, 146, 251-259.
(38) Burslem, G. M.; Smith, B. E.; Lai, A. C.; Jaime-Figueroa, S.; McQuaid, D. C.; Bondeson, D. P.; Toure, M.; Dong, H.; Qian, Y.; Wang, J.; Crew, A. P.; Hines, J.; Crews, C. M. The advantages of targeted protein degradation over inhibition: an RTK case study. Cell Chem. Biol. 2018, 25, 67-77.
(39) Schiedel, M.; Herp, D.; Hammelmann, S.; Swyter, S.; Lehotzky, A.; Robaa, D.; Olah, J.; Ovadi, J.; Sippl, W.; Jung, M. Chemically induced degradation of sirtuin 2 (sirt2) by a proteolysis targeting chimera (PROTAC) based on sirtuin rearranging ligands (SirReals). J. Med Chem. 2018, 61, 482-491.
(40) Shibata, N.; Nagai, K.; Morita, Y.; Ujikawa, O.; Ohoka, N.; Hattori, T.; Koyama, R.; Sano, O.; Imaeda, Y.; Nara, H.; Cho, N.; Naito, M. Development of protein degradation inducers of androgen receptor by conjugation of androgen receptor ligands and inhibitor of apoptosis protein ligands. J. Med Chem. 2018, 61, 543-575.
(41) Crew, A. P.; Raina, K.; Dong, H.; Qian, Y.; Wang, J.; Vigil, D.; Serebrenik, Y. V.; Hamman, B. D.; Morgan, A.; Ferraro, C.; Siu, K.; Neklesa, T. K.; Winkler, J. D.; Coleman, K. G.; Crews, C. M. Identification and characterization of von Hippel-Lindau-recruiting proteolysis targeting chimeras (PROTACs) of TANK-binding kinase 1. J Med Chem. 2018, 61, 583-598.
(42) Sun, Y.; Zhao, X.; Ding, N.; Gao, H.; Wu, Y.; Yang, Y.; Zhao, M.; Hwang, J.; Song, Y.; Liu, W.; Rao, Y. PROTAC-induced BTK degradation as a novel therapy for mutated BTK C481 S induced ibrutinib-resistant B-cell malignancies. Cell Res. 2018, 28, 779-781.
(43) Yang, K.; Song, Y.; Xie, H.; Wu, H.; Wu, Y. T.; Leisten, E. D.; Tang, W. Development of the first small molecule histone deacetylase 6 (HDAC6) degraders. Bioorg. Med Chem. Lett. 2018, 28, 2493-2497.
(44) Ohoka, N.; Okuhira, K.; Ito, M.; Nagai, K.; Shibata, N.; Hattori, T.; Ujikawa, O.; Shimokawa, K.; Sano, O.; Koyama, R.; Fujita, H.; Teratani, M.; Matsumoto, H.; Imaeda, Y.; Nara, H.; Cho, N.; Naito, M. In vivo knockdown of pathogenic proteins via specific and nongenetic inhibitor of apoptosis protein (IAP)-dependent protein erasers (SNIPERs). J. Biol. Chem. 2017, 292, 4556-4570.
(45) Ohoka, N.; Morita, Y.; Nagai, K.; Shimokawa, K.; Ujikawa, O.; Fujimori, I.; Ito, M.; Hayase, Y.; Okuhira, K.; Shibata, N.; Hattori, T.; Sameshima, T.; Sano, O.; Koyama, R.; Imaeda, Y.; Nara, H.; Cho, N.; Naito, M. Derivatization of inhibitor of apoptosis protein (IAP) ligands yields improved inducers of estrogen receptor a degradation. J. Biol. Chem. 2018, 293, 6776-6790.
(46) Ottis, P.; Toure, M.; Cromm, P. M.; Ko, E.; Gustafson, J. L.; Crews, C. M. Assessing different E3 ligases for small molecule induced protein ubiquitination and degradation. ACS Chem. Biol. 2017, 12, 2570-2578.
(47) Kanak Rainaa, J. L., Yimin Qiana, Martha Altieria, Deborah Gordona, Ann Marie K. Rossia, Jing Wanga, Xin Chena, Hanqing Donga, Kam Siua, James D. Winklera, Andrew P. Crewa, Craig M. Crews and Kevin G. Colemana. PROTAC-induced BET protein degradation as a therapy for castration-resistant prostate cancer. Proc. Natl. Acad. Sci. U.S.A 2016, 113, 7124-7129.
(48) Zhou, B.; Hu, J.; Xu, F.; Chen, Z.; Bai, L.; Fernandez-Salas, E.; Lin, M.; Liu, L.; Yang, C. Y.; Zhao, Y.; McEachern, D.; Przybranowski, S.; Wen, B.; Sun, D.; Wang, S. Discovery of a small-molecule degrader of bromodomain and extra-terminal (BET) proteins with picomolar cellular potencies and capable of achieving tumor regression. J. Med. Chem. 2018, 61, 462-481.
(49) Andrzej M. Brzozowski, A. C. W. P., Zbigniew Dauter, Roderick E. Hubbard, Tomas Bonn, Owe Engstro, Lars O″ hman, Geoffrey L. Greene, Jan-A^{∘} ke Gustafsson,Mats Carlquist. Molecular basis of agonism and antagonism in the oestrogen receptor. Nature 1997, 389, 753-758.
(50) Qin, C.; Hu, Y.; Zhou, B.; Fernandez-Salas, E.; Yang, C. Y.; Liu, L.; McEachern, D.; Przybranowski, S.; Wang, M.; Stuckey, J.; Meagher, J.; Bai, L.; Chen, Z.; Lin, M.; Yang, J.; Ziazadeh, D. N.; Xu, F.; Hu, J.; Xiang, W.; Huang, L.; Li, S.; Wen, B.; Sun, D.; Wang, S. Discovery of QCA570 as an exceptionally potent and efficacious proteolysis targeting chimera (PROTAC) degrader of the bromodomain and extra-terminal (BET) proteins capable of inducing complete and durable tumor regression. J. Med. Chem. 2018, 61, 6685-6704.
(51) Bai, L.; Zhou, B.; Yang, C. Y.; Ji, J.; McEachern, D.; Przybranowski, S.; Jiang, H.; Hu, J.; Xu, F.; Zhao, Y.; Liu, L.; Fernandez-Salas, E.; Xu, J.; Dou, Y.; Wen, B.; Sun, D.; Meagher, J.; Stuckey, J.; Hayes, D. F.; Li, S.; Ellis, M. J.; Wang, S. Targeted degradation of BET proteins in triple-negative breast cancer. Cancer Res. 2017, 77, 2476-2487.
(52) Buckley, D. L.; Van Molle, I.; Gareiss, P. C.; Tae, H. S.; Michel, J.; Noblin, D. J.; Jorgensen, W. L.; Ciulli, A.; Crews, C. M. Targeting the von Hippel-Lindau E³ ubiquitin ligase using small molecules to disrupt the VHL/HIF-la interaction. J. Am. Chem. Soc. 2012, 134, 4465-4468.
(53) Buckley, D. L.; Gustafson, J. L.; Van Molle, I.; Roth, A. G.; Tae, H. S.; Gareiss, P. C.; Jorgensen, W. L.; Ciulli, A.; Crews, C. M. Small-molecule inhibitors of the interaction between the E3 ligase VHL and HIF-la. Angew. Chem., Int. Ed. 2012, 51, 11463-11467.
(54) Galdeano, C.; Gadd, M. S.; Soares, P.; Scaffidi, S.; Van Molle, I.; Birced, I.; Hewitt, S.; Dias, D. M.; Ciulli, A. Structure-guided design and optimization of small molecules targeting the protein-protein interaction between the von Hippel-Lindau (VHL) E3 ubiquitin ligase and the hypoxia inducible factor (HIF) a subunit with in vitro nanomolar affinities. J. Med. Chem. 2014, 57, 8657-8663.
(55) Soares, P.; Gadd, M. S.; Frost, J.; Galdeano, C.; Ellis, L.; Epemolu, O.; Rocha, S.; Read, K. D.; Ciulli, A. Group-based optimization of potent and cell-active inhibitors of the von Hippel-Lindau (VHL) E3 ubiquitin ligase: structure-activity relationships leading to the chemical probe (2S,4R)-1-((S)-2-(1-cyanocyclopropanecarboxamido)-3,3-dimethylbutanoyl)-4-hydroxy-N-(4-(4-methylthiazol-5-yl)benzyl)pyrrolidine-2-carboxamide (VH298). J. Med. Chem. 2018, 61, 599-618.
(56) Gadd, M. S.; Testa, A.; Lucas, X.; Chan, K. H.; Chen, W.; Lamont, D. J.; Zengerle, M.; Ciulli, A. Structural basis of PROTAC cooperative recognition for selective protein degradation. Nat. Chem. Biol. 2017, 13, 514-521.
(57) Long, M. J.; Poganik, J. R.; Aye, Y. On-demand targeting: investigating biology with proximity-directed chemistry. J. Am. Chem. Soc. 2016, 138, 3610-3622.
(58) Stols, L.; Gu, M.; Dieckman, L.; Raffen, R.; Collart, F. R.; Donnelly, M. I. A new vector for high-throughput, ligation-independent cloning encoding a tobacco etch virus protease cleavage site. Protein Expr. Purif 2002, 25, 8-15.
(59) Benoit, R. M.; Ostermeier, C.; Geiser, M.; Li, J. S.; Widmer, H.; Auer, M. Seamless insert-plasmid assembly at high efficiency and low cost. PLoS One 2016, 11, e0153158.

### EQUIVALENTS

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I:
A-X-J-Y—Z—(CH₂)ₙ—B¹ I,
or a pharmaceutically acceptable salt or solvate thereof, wherein:
A is selected from: 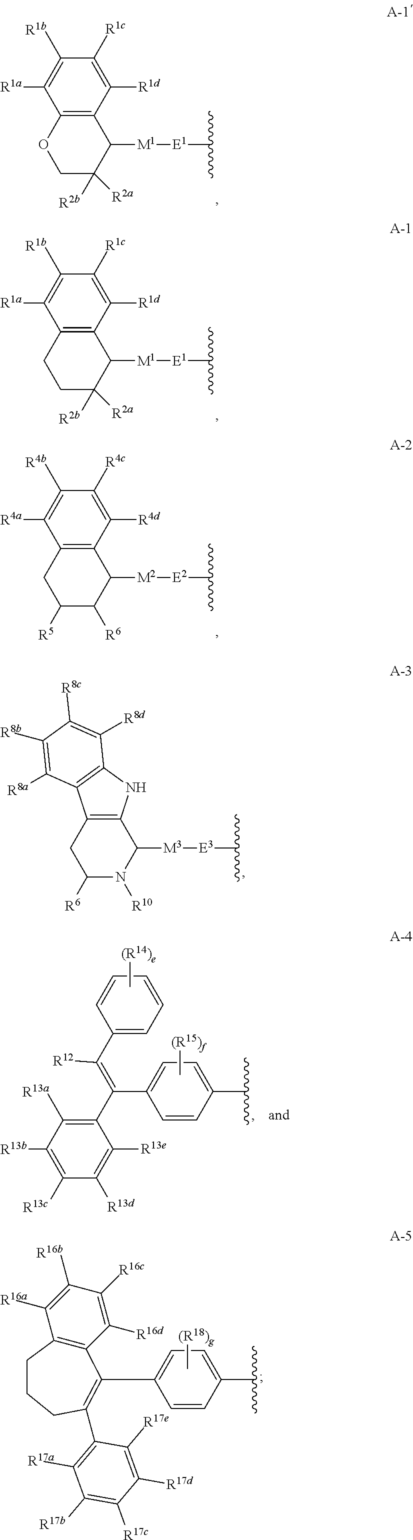
M¹ is selected from 4- to 8-membered heterocyclenyl, phenylenyl, 5-membered heteroarylenyl, and 6-membered heteroarylenyl, wherein the 4- to 8-membered heterocyclenyl, phenylenyl, 5-membered heteroarylenyl, or 6-membered heteroarylenyl is optionally substituted with one or more halo or C₁-C₃ alkoxy;
R^{1a}, R^{1b}, R^{1c}, and R^{1d} are independently selected from hydrogen, halo, hydroxy, 5-membered heteroayl, and —B(OH)₂, wherein the 5-membered heteroayl is optionally substituted with one or more C₁-C₄ alkyl; or
R^{1a} and R^{1b} taken together with the carbon atom to which they are attached form an optionally substituted 5- or 6-membered heteroaryl; and R^{1c} and R^{1d} are hydrogen;
R^{2a} is selected from optionally substituted phenyl and optionally substituted C₃-C₈ cycloalkyl; and R^{2b} is hydrogen; or
R^{2a} and R^{2b} taken together with the carbon atom to which they are attached form a C₃-C₈ cycloalkyl;
E¹ is selected from —C(═O)—, —C≡C—, —O—, —O—(CH₂)_{b}—, N(R^{3e})—, and —(CH₂)_{b}—;
R^{3e} is selected from hydrogen and C₁-C₄ alkyl;
b is 0, 1, 2, 3, 4, or 5;
M² is selected from 4- to 8-membered heterocyclenyl, phenylenyl, 5-membered heteroarylenyl, and 6-membered heteroarylenyl, wherein the 4- to 8-membered heterocyclenyl, phenylenyl, 5-membered heteroarylenyl, or 6-membered heteroarylenyl is optionally substituted with one or more halo or C₁-C₃ alkoxy;
R^{4a}, R^{4b}, R^{4c}, and R^{4d} are independently selected from hydrogen, halo, hydroxy, and —B(OH)₂; or
R^{4a} and R^{4b} taken together with the carbon atoms to which they are attached form an optionally substituted 5- or 6-membered heteroaryl; and R^{4c} and R^{4d} are hydrogen;
R⁵ is C₁-C₃ alkyl;
R⁶ is C₁-C₄ haloalkyl;
E² is selected from —C(═O)—, —C≡C—, —O—, —O—(CH₂)_{c}—, —N(R^{7e})—, and —(CH₂)_{c}—;
R^{7e} is selected from hydrogen and C₁-C₄ alkyl;
c is 0, 1, 2, 3, 4, or 5;
M³ is selected from 4- to 8-membered heterocyclenyl, phenylenyl, 5-membered heteroarylenyl, and 6-membered heteroarylenyl, wherein the 4- to 8-membered heterocyclenyl, phenylenyl, 5-membered heteroarylenyl, or 6-membered heteroarylenyl is optionally substituted with one or more halo or C₁-C₃ alkoxy;
R^{8a}, R^{8b}, R^{8c}, and R^{8d} are independently selected from hydrogen, halo, hydroxy, and —B(OH)₂;
R⁹ is C₁-C₃ alkyl;
R¹⁰ is C₁-C₄ haloalkyl optionally substituted with one or more hydroxy, or —(C₁-C₄ alkyl)-(C₃-C₈ cycloalkyl) optionally substituted with one or more halo;
E³ is selected from —C(═O)—, —C≡C—, —O—, —O—(CH₂)_{d}—, N(R^{11e})—, and —(CH₂)_{d}—;
R^{11e} is selected from hydrogen and C₁-C₄ alkyl;
d is 0, 1, 2, 3, 4, or 5;
R¹² is selected from C₁-C₄ alkyl and C₁-C₄ haloalkyl;
R^{13a}, R^{13b}, R^{13c}, and R^{13d} are independently selected from hydrogen, halo, hydroxy, and —B(OH)₂;
each R¹⁴ is independently selected from hydrogen, halo, and hydroxy;
e is 0, 1, 2, or 3;
each R¹⁵ is independently selected from hydrogen, halo, and hydroxy;
f is 0, 1, 2, or 3;
R^{16a}, R^{16b}, R^{16c}, and R^{16d} are independently selected from hydrogen, halo, hydroxy, and —B(OH)₂;
R^{17a}, R^{17b}, R^{17c}, R^{17d}, and R^{17e} are independently selected from hydrogen, halo, and hydroxy;
each R¹⁸ is independently selected from hydrogen, halo, and hydroxy;
g is 0, 1, 2, or 3;
X is selected from cycloalkylenyl, heterocyclenyl, phenylenyl, and heteroarylenyl, wherein the cycloalkylenyl, heterocyclenyl, phenylenyl, or heteroarylenyl is optionally substituted with one or more C₁-C₄ alkyl;
J is selected from —C(═O)—, —(CH₂)ₘ—, —(CH₂)_{z1}N(R¹⁹)—, and —(CH₂)_{z2}O—,
m is 0, 1, 2, or 3;
z1 is 0, 1, or 2;
z2 is 0, 1, or 2;
R¹⁹ is selected from hydrogen and C₁-C₄ alkyl;
Y is selected from cycloalkylenyl, heterocyclenyl, heteroarylenyl, —C(═O)— and —(CR^{20a}R^{20b})ᵣ—;
Z is selected from cycloalkylenyl, heterocyclenyl, heteroarylenyl, —C(═O)— and —(CR^{20c}R^{20d})ₛ—;
each R^{20a}, R^{20b}, R^{20c}, and R^{20d} is independently selected from hydrogen and C₁-C₃ alkyl;
r is 0, 1, 2, 3, 4, or 5;
s is 0, 1, 2, 3, 4, or 5;
with the provisos:
(i) Z is cycloalkylenyl, heterocyclenyl, heteroarylenyl, or —(CR^{20c}R^{20d})ₛ— when Y is —C(═O)—; or
(ii) Y is cycloalkylenyl, heterocyclenyl, heteroarylenyl, or —(CR^{20a}R^{20b})ᵣ— when Z is —C(═O)—;
n is 0, 1, 2, or 3;
B¹ is selected from hydrogen, hydroxy, 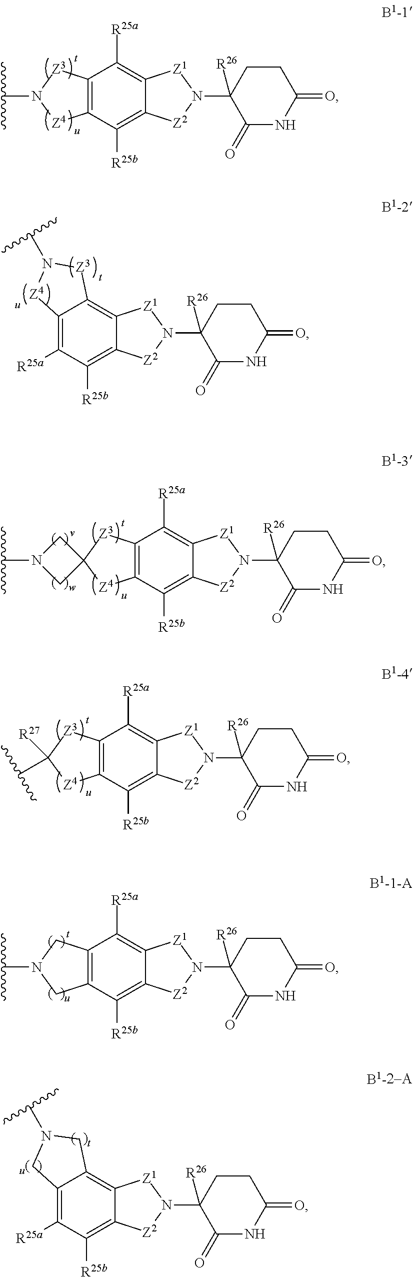 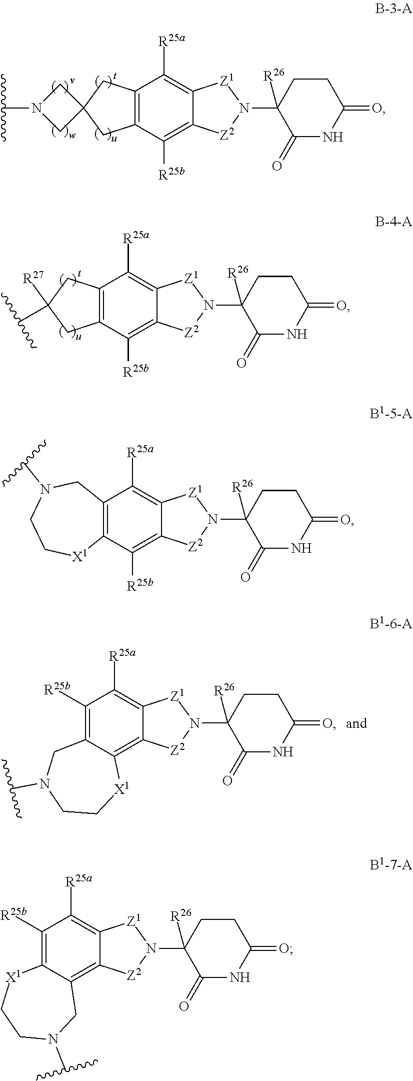
R^{25a} and R^{25b} are independently selected from hydrogen, amino, halo, C₁-C₃ alkyl, and C₁-C₃ alkoxy;
R²⁶ is selected from hydrogen, deuterium, fluoro, and C₁-C₃ alkyl;
R²⁷ is selected from hydrogen and C₁-C₃ alkyl;
Z¹ and Z² are independently selected from —C(═O)— and —CR^{28a}R^{28b}—;
with the provisos:
(iv) one of Z¹ or Z² is —C(═O)—; or
(v) both of Z¹ and Z² are —C(═O)—;
R^{28a} and R^{28b} are independently selected from hydrogen and C₁-C₃ alkyl; or
R^{28a} and R^{28b} taken together with the carbon atom to which they are attached form a C₃-C₆ cycloalkyl;
Z³ and Z⁴ are independently selected from —C(═O)— and —CR^{28c}R^{28d}—;
with the provisos:
(iv) one of Z³ or Z⁴ is —C(═O)—; or
(v) both of Z³ and Z⁴ are —C(═O)—;
R^{28c} and R^{28d} are independently selected from hydrogen and C₁-C₃ alkyl; or
R^{28c} and R^{28d} taken together with the carbon atom to which they are attached form a C₃-C₆ cycloalkyl;
X¹ is selected from —O—, —S—, and —N(R²⁹)—;
R²⁹ is selected from hydrogen and C₁-C₄ alkyl;
t is 1, 2, or 3;
u is 1, 2, or 3;
v is 1, 2, or 3; and
w is 1, 2, or 3.

2. The compound of claim 1, or a pharmaceutically acceptable salt or solvate thereof, wherein:
A is selected from: 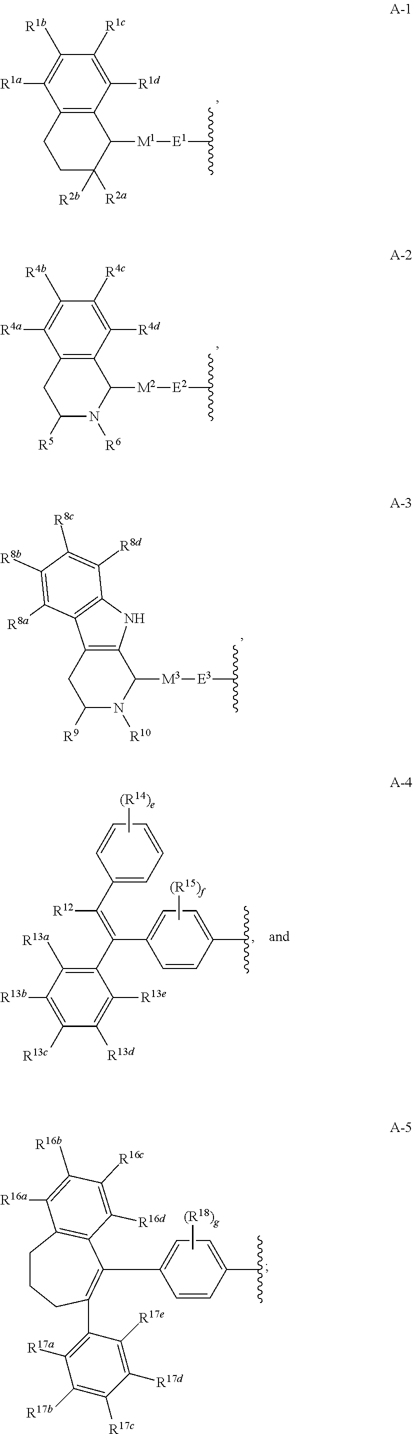
M¹ is selected from 4- to 8-membered heterocyclenyl, phenylenyl, 5-membered heteroarylenyl, and 6-membered heteroarylenyl;
R^{1a}, R^{1b}, R^{1c}, and R^{1d} are independently selected from hydrogen, halo, hydroxy, and —B(OH)₂;
R^{2a} is selected from optionally substituted phenyl and optionally substituted C₃-C₈ cycloalkyl; and R^{2b} is hydrogen; or
R^{2a} and R^{2b} taken together with the carbon atom to which they are attached form a C₃-C₈ cycloalkyl;
E¹ is selected from —C≡C—, —O—, N(R^{3e})—, and —(CH₂)_{b}—;
R^{3e} is selected from hydrogen and C₁-C₄ alkyl;
b is 0, 1, 2, 3, 4, or 5;
M² is selected from 4- to 8-membered heterocyclenyl, phenylenyl, 5-membered heteroarylenyl, and 6-membered heteroarylenyl;
R^{4a}, R^{4b}, R^{4c}, and R^{4d} are independently selected from hydrogen, halo, hydroxy, and —B(OH)₂; or
R^{4a} and R^{4b} taken together with the carbon atoms to which they are attached form an optionally substituted 5- or 6-membered heteroaryl; and R^{4c} and R^{4d} are hydrogen;
R⁵ is C₁-C₃ alkyl;
R⁶ is C₁-C₄ haloalkyl;
E² is selected from —C≡C—, —O—, —N(R^{7e})—, and —(CH₂)_{c}—;
R^{7e} is selected from hydrogen and C₁-C₄ alkyl;
c is 0, 1, 2, 3, 4, or 5;
M³ is selected from 4- to 8-membered heterocyclenyl, phenylenyl, 5-membered heteroarylenyl, and 6-membered heteroarylenyl;
R^{8a}, R^{8b}, R^{8c}, and R^{8d} are independently selected from hydrogen, halo, hydroxy, and —B(OH)₂;
R⁹ is C₁-C₃ alkyl;
R¹⁰ is C₁-C₄ haloalkyl;
E³ is selected from —C≡C—, —O—, N(R^{11e})—, and —(CH₂)_{d}—;
R^{11e} is selected from hydrogen and C₁-C₄ alkyl;
d is 0, 1, 2, 3, 4, or 5;
R¹² is selected from C₁-C₄ alkyl and C₁-C₄ haloalkyl;
R^{13a}, R^{13b}, R^{13c}, and R^{13d} are independently selected from hydrogen, halo, hydroxy, and —B(OH)₂;
each R¹⁴ is independently selected from hydrogen, halo, and hydroxy;
e is 0, 1, 2, or 3;
each R¹⁵ is independently selected from hydrogen, halo, and hydroxy;
f is 0, 1, 2, or 3;
R^{16a}, R^{16b}, R^{16c}, and R^{16d} are independently selected from hydrogen, halo, hydroxy, and —B(OH)₂;
R^{17a}, R^{17b}, R^{17c}, R^{17d}, and R^{17e} are independently selected from hydrogen, halo, and hydroxy;
each R¹⁸ is independently selected from hydrogen, halo, and hydroxy;
g is 0, 1, 2, or 3;
X is selected from cycloalkylenyl, heterocyclenyl, phenylenyl, and heteroarylenyl;
J is selected from —(CH₂)ₘ—, —(CH₂)_{z1}N(R¹⁹)—, and —(CH₂)_{z2}O—,
m is 0, 1, 2, or 3;
z1 is 0, 1, or 2;
z2 is 0, 1, or 2;
R¹⁹ is selected from hydrogen and C₁-C₄ alkyl;
Y is selected from cycloalkylenyl, heterocyclenyl, heteroarylenyl, —C(═O)— and —(CR^{20a}R^{20b})ᵣ—;
Z is selected from cycloalkylenyl, heterocyclenyl, heteroarylenyl, —C(═O)— and —(CR^{20c}R^{20d})ₛ—;
each R^{20a}, R^{20b}, R^{20c}, and R^{20d} is independently selected from hydrogen and C₁-C₃ alkyl;
r is 0, 1, 2, 3, 4, or 5;
s is 0, 1, 2, 3, 4, or 5;
with the provisos:
(i) Z is —(CR^{20c}CR^{20d})ₛ— when Y is —C(═O)—; or
(ii) Y is —(CR^{20a}R^{20b})ᵣ— when Z is —C(═O)—;
n is 0, 1, 2, or 3;
B¹ is selected from hydrogen, hydroxy, 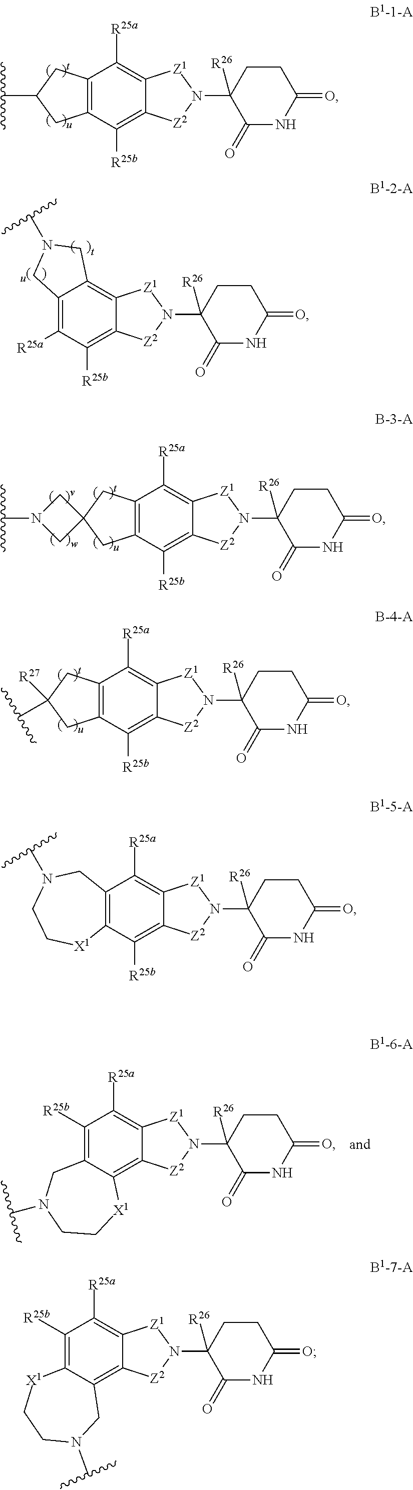
R^{25a} and R^{25b} are independently selected from hydrogen, amino, halo, C₁-C₃ alkyl, and C₁-C₃ alkoxy;
R²⁶ is selected from hydrogen, deuterium, fluoro, and C₁-C₃ alkyl;
R²⁷ is selected from hydrogen and C₁-C₃ alkyl;
Z¹ and Z² are independently selected from —C(═O)— and —CR^{28a}R^{28b}—;
with the provisos:
(iv) one of Z¹ or Z² is —C(═O)—; or
(v) both of Z¹ and Z² are —C(═O)—;
R^{28a} and R^{28b} are independently selected from hydrogen and C₁-C₃ alkyl; or
R^{28a} and R^{28b} taken together with the carbon atom to which they are attached form a C₃-C₆ cycloalkyl;
X¹ is selected from —O—, —S—, and —N(R²⁹)—;
R²⁹ is selected from hydrogen and C₁-C₄ alkyl;
t is 1, 2, or 3;
u is 1, 2, or 3;
v is 1, 2, or 3; and
w is 1, 2, or 3.

3. The compound of any one of the preceding claims, or a pharmaceutically acceptable salt or solvate thereof, wherein A is A-1′ or A-1; and
optionally; M¹ is selected from 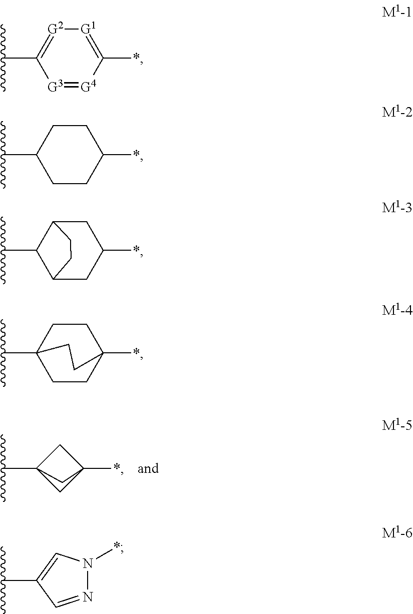
wherein the bond designated with an “*” is attached to E¹; G¹ is selected from —N═ and —CR^{3a}═; G² is selected from —N═ and —CR^{3b}═; G³ is selected from —N═ and —CR^{3c}═; G⁴ is selected from —N═ and —CR^{3d}═; and R^{3a}, R^{3b}, R^{3c}, R^{3d} are independently selected from hydrogen and halo, with the proviso that E¹ is —(CH₂)_{b}— when M¹ is M¹-6; and
optionally; M¹ is M¹-1.

4. The compound of any one of the preceding claims, or a pharmaceutically acceptable salt or solvate thereof, wherein A is A-2; and
optionally; M² is selected from 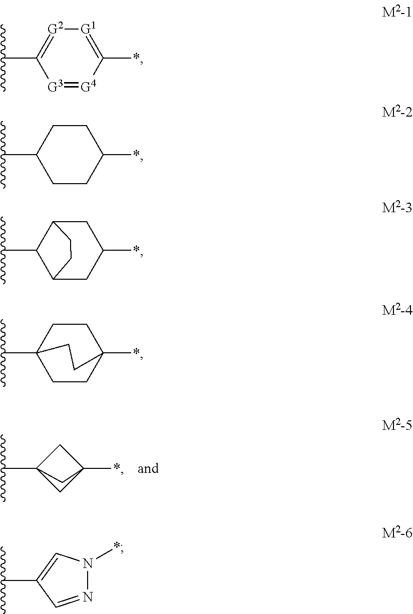
wherein the bond designated with an “*” is attached to E²; G⁵ is selected from —N═ and —CR^{7a}═; G⁶ is selected from —N═ and —CR^{7b}═; G⁷ is selected from —N═ and —CR^{7c}═; G⁸ is selected from —N═ and —CR^{7d}═; and R^{7a}, R^{7b}, R^{7c}, and R^{7d} are independently selected from hydrogen and halo, with the proviso that E² is —(CH₂)_{c}— when M² is M²-6; and
optionally; M² is M²-1.

5. The compound of any one of the preceding claims, or a pharmaceutically acceptable salt or solvate thereof, wherein A is A-3; and
optionally; M³ is selected from 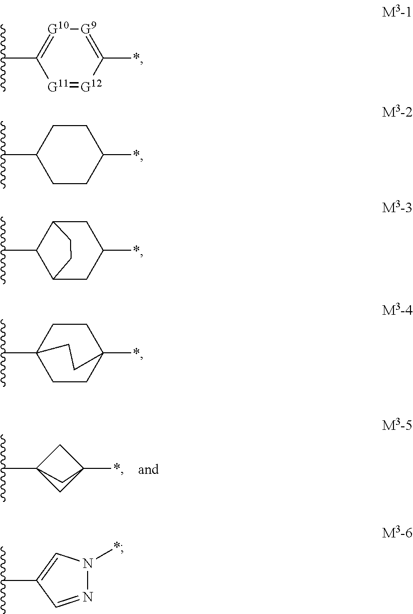
wherein the bond designated with an “*” is attached to E³; G⁹ is selected from —N═ and —CR^{11a}═; G¹⁰ is selected from —N═ and —CR^{11b}═; G¹¹ is selected from —N═ and —CR^{11c}═; G¹² is selected from —N═ and —CR^{11d}═; and R^{11a}, R^{11b}, R^{11c}, and R^{11d} are independently selected from hydrogen and halo, with the proviso that E³ is —(CH₂)_{d}— when M² is M³-6; and
optionally; M³ is M³-1.

6. The compound of any one of the preceding claims, or a pharmaceutically acceptable salt or solvate thereof, wherein A is A-4 or A-5.

7. The compound of any one of the preceding claims, being of Formula II or III: 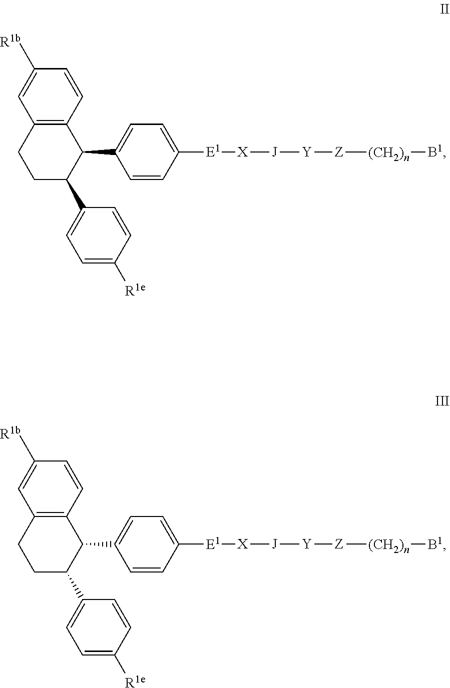
or a pharmaceutically acceptable salt or solvate thereof, wherein R^{1e} is selected from hydrogen and halo; and
optionally, R^{1b} is hydroxy and R^{1e} is hydrogen or fluoro; and
optionally, R^{1e} is hydrogen; and
optionally, E¹ is —O—; or E¹ is —(CH₂)_{b}— and b is 0.

8. The compound of any one of the preceding claims, being of Formula IV or V: 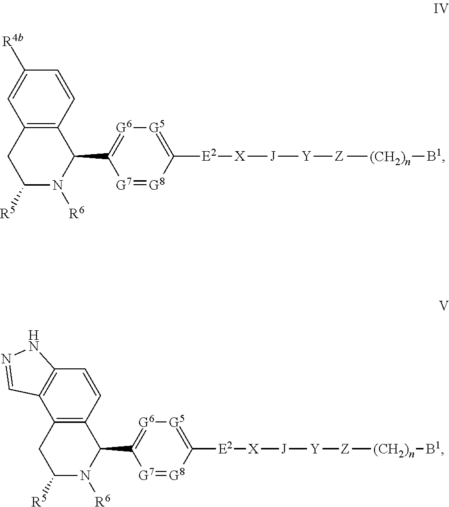
or a pharmaceutically acceptable salt or solvate thereof; and
optionally, R^{4b} is hydroxy; and
optionally, R⁵ is methyl; and
optionally, R⁶ is selected from —CH₂CF₂CH₃, —CH₂CF₂H, and —CH₂CF₃; and
optionally, E² is —(CH₂)_{c}— and c is 0.

9. The compound of any one of the preceding claims, being of Formula VI: 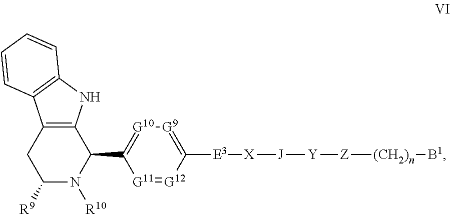
or a pharmaceutically acceptable salt or solvate thereof; and
optionally, R⁹ is methyl; and
optionally, R¹⁰ is selected from —CH₂CF₂CH₃, —CH₂CF₂H, and —CH₂CF₃; and
optionally, E³ is —(CH₂)_{d}— and d is 0.

10. The compound of any one of the preceding claims, being of Formula VII: 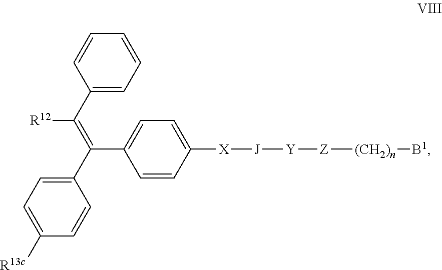
or a pharmaceutically acceptable salt or solvate thereof; and
optionally, R¹² is selected from —CH₂CH₃ and —CH₂CH₂C₁; and
optionally, R^{13c} is hydroxy.

11. The compound of any one of the preceding claims, being of Formula VII: 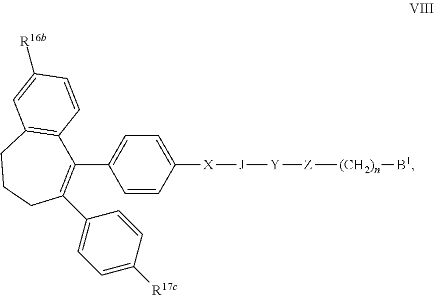
or a pharmaceutically acceptable salt or solvate thereof; and
optionally, R^{6b} is hydroxy; and
optionally, R^{17c} is fluoro.

12. The compound of any one of the preceding claims, or a pharmaceutically acceptable salt or solvate thereof, wherein X is heterocyclenyl; and
optionally, wherein:
(i) X is optionally substituted 4- to 8-membered heterocyclenyl; and
optionally, X is selected from 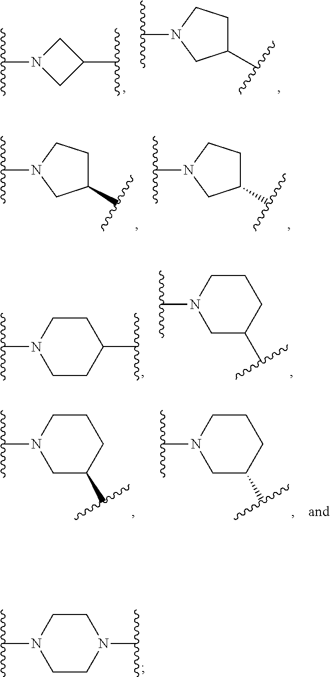
or
(ii) X is a 7- to 14-membered spiroheterocyclenyl; and
optionally, X is 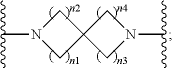
and n₁, n₂, n₃, and n₄ are independently 0, 1, 2, 3, or 4, with the proviso that the sum of n₁, n₂, n₃, and n₄ is 4, 5, 6, 7, 8, 9, or 10; and
optionally, X is selected from

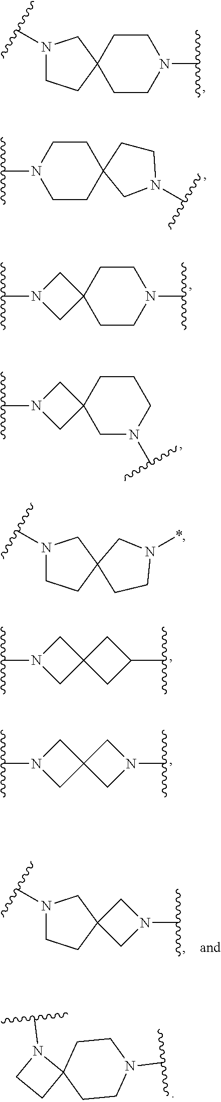

13. The compound of any one of the preceding claims, or a pharmaceutically acceptable salt or solvate thereof, wherein J is —(CH₂)ₘ— and m is 0 or 1; and
optionally, m is 0.

14. The compound of any one of the preceding claims, or a pharmaceutically acceptable salt or solvate thereof, wherein Y is selected from heterocyclenyl, —C(═O)— and —(CH₂)ᵣ—; and
optionally, wherein:
(i) Y is optionally substituted 4- to 8-membered heterocyclenyl; and
optionally, Y is 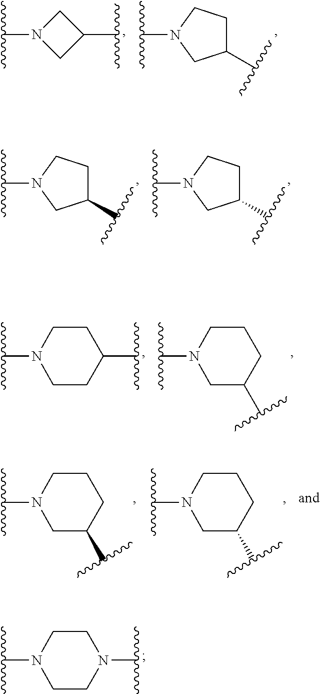
(ii) Y is —(CH₂)ᵣ—; or
(iii) Y is —C(═O)—.

15. The compound of any one of the preceding claims, or a pharmaceutically acceptable salt or solvate thereof, wherein Z is selected from heterocyclenyl and —(CH₂)ₛ—; and
optionally, wherein:
(i) Z is optionally substituted 4- to 8-membered heterocyclenyl; and
optionally, Z is 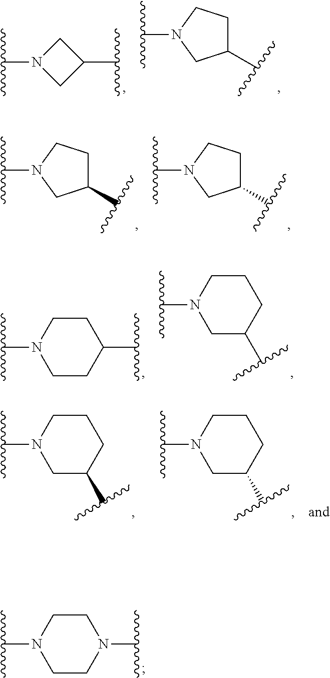
(ii) Z is —(CH₂)ₛ—; or
(iii) Z is —C(═O)—.

16. The compound of any one of the preceding claims, or a pharmaceutically acceptable salt or solvate thereof, wherein n is 0 or 1.

17. The compound of any one of the preceding claims, or a pharmaceutically acceptable salt or solvate thereof, wherein B¹ is B¹-1-A, B¹-1-B, or B¹-1-C; and
optionally, t is 1 or 2; and
optionally, u is 1; or u is 2.

18. The compound of any one of the preceding claims, or a pharmaceutically acceptable salt or solvate thereof, wherein B¹ is B¹-2-A, B¹-2-B, or B¹-2-C; and
optionally, t is 1 or 2; and
optionally, u is 1; or u is 2.

19. The compound of any one of the preceding claims, or a pharmaceutically acceptable salt or solvate thereof, wherein B¹ is B¹-3-A, B¹-3-B, or B¹-3-C; and
optionally, t is 1 or 2; and
optionally, u is 1; or u is 2; and
optionally, v is 1; and
optionally, w is 1; or w is 2.

20. The compound of any one of the preceding claims, or a pharmaceutically acceptable salt or solvate thereof, wherein B¹ is B¹-4-A, B¹-4-B, or B¹-4-C.
optionally, t is 1 or 2; and
optionally, u is 1; or u is 2; and
optionally, R²⁷ is hydrogen.

21. The compound of any one of the preceding claims, or a pharmaceutically acceptable salt or solvate thereof, wherein B¹ is B¹-5-A, B¹-5-B, B¹-5-C, B¹-6-A, B¹-6-B, B¹-6-C, B¹-7-A, B¹-7-B, or B¹-7-C; and
optionally, X¹ is —O—.

22. The compound of any one of the preceding claims, or a pharmaceutically acceptable salt or solvate thereof, wherein R^{25a} is hydrogen; and
optionally, R^{25b} is hydrogen; and
optionally, R²⁶ is hydrogen; and
optionally, Z¹ is —C(═O)—; or Z¹ is —CH₂—; and
optionally, Z² is —C(═O)—.

23. The compound of any one of the preceding claims, being selected from the compounds described in Table 1, and pharmaceutically acceptable salts and solvates thereof.

24. The compound of any one of the preceding claims, or a salt or solvate thereof, wherein B¹ is selected from hydrogen or hydroxy;
optionally, wherein the compound is selected from the compounds described in Table 2, and pharmaceutically acceptable salts and solvates thereof.

25. A pharmaceutical composition comprising the compound of any one of the preceding claims, or a pharmaceutically acceptable salt or solvate thereof, and a pharmaceutically acceptable carrier.

26. A method of degrading an ER protein in a subject, comprising administering to the subject the compound of any one of the preceding claims or a pharmaceutically acceptable salt or solvate thereof.

27. Use of the compound of any one of the preceding claims or a pharmaceutically acceptable salt or solvate thereof in the manufacture of a medicament for degrading an ER protein in a subject.

28. The compound of any one of the preceding claims or a pharmaceutically acceptable salt or solvate thereof for use in degrading an ER protein in a subject.

29. A method of treating or preventing a disease in a subject in need thereof, comprising administering to the subject the compound of any one of the preceding claims or a pharmaceutically acceptable salt or solvate thereof

30. Use of the compound of any one of the preceding claims or a pharmaceutically acceptable salt or solvate thereof in the manufacture of a medicament for treating or preventing a disease in a subject.

31. The compound of any one of the preceding claims or a pharmaceutically acceptable salt or solvate thereof for use in treating or preventing a disease in a subject.

32. The method, use, or compound for use in any one of the preceding claims, wherein the subject is a mammal.

33. The method, use, or compound for use in any one of the preceding claims, wherein the subject is a human.

34. The method, use, or compound for use in any one of the preceding claims, wherein the disease is associated with degradation of an ER protein.

35. The method, use, or compound for use in any one of the preceding claims, wherein the disease is a cancer;
optionally, the cancer is selected from the cancers described in Table I; and
optionally, the cancer is breast cancer.
